(19) United States
(12) Patent Application Publication
(10) Pub. No.: US 2026/0184711 A1
(43) Pub. Date: Jul. 2, 2026

(54) TETHERED HETEROCYCLIC INHIBITORS OF KRAS G12C MUTANT PROTEINS AND USES THEREOF

(71) Applicant: AMGEN INC., THOUSAND OAKS, CA (US)

(72) Inventors: BRIAN A. LANMAN, WOODLAND HILLS, CA (US); ADILI ALAFATE, NEWBBURY PARK, CA (US); ABHISEK BANERJEE, KARNATAKA (IN); EMIL GLIBSTRUP, COPENHAGEN (DK); IMELDA HOT, THOUSAND OAKS, CA (US); DAVID HUANG, THOUSAND OAKS, CA (US); BIRGITTE W. HUSEMOEN, COPENHAGEN (DK); MATTHEW R. KALLER, THOUSAND OAKS, CA (US); PATRICIA LOPEZ, WOODLAND HILLS, CA (US); VU VAN MA, OAK PARK, CA (US); FRANCESCO MANONI, NEWBURY PARK, CA (US); SLAVKO RAST, VALBY (DK); NURIA A. TAMAYO, NEWBURY PARK, CA (US); HUI-LING WANG, THOUSAND OAKS, CA (US); JINGJING XIE, THOUSAND OAKS, CA (US); WENHAN ZHANG, THOUSAND OAKS, CA (US)

(73) Assignee: AMGEN INC., THOUSAND OAKS, CA (US)

(21) Appl. No.: 19/118,048

(22) PCT Filed: Oct. 5, 2023
(86) PCT No.: PCT/US2023/034533

§ 371 Date: Apr. 3, 2025

Related U.S. Application Data
(60) Provisional application No. 63/413,548, filed on Oct. 5, 2022.

(51) Int. Cl. C07D 471/14; A61K 31/496; A61K 31/55; A61K 31/553; A61K 45/06; A61P 35/00; C07B 59/00; C07D 205/04; C07D 213/61; C07D 213/74; C07D 231/16; C07D 401/04; C07D 401/14; C07D 403/04; C07D 405/04; C07D 405/14; C07D 417/14; C07D 487/10; C07D 487/14; C07D 491/107; C07D 498/14; C07D 519/00; C07F 5/02

(52) U.S. Cl. CPC C07D 471/14; A61K 31/496; A61K 31/55; A61K 31/553; A61K 45/06; A61P 35/00; C07B 59/002; C07B 59/004; C07D 205/04; C07D 213/61; C07D 213/74; C07D 231/16; C07D 401/04; C07D 401/14; C07D 403/04; C07D 405/04; C07D 405/14; C07D 417/14; C07D 487/10; C07D 487/14; C07D 491/107; C07D 498/14; C07D 519/00; C07F 5/025; C07B 2200/05

(57) ABSTRACT

The present disclosure provides compounds having activity as inhibitors of the G12C mutant KRAS protein, pharmaceutical compositions comprising the compounds, and methods of treating certain disorders, such as cancer, including but not limited to lung, pancreatic, and colorectal cancer. In particular, the disclosure provides compounds of Formula (II) and pharmaceutically acceptable salts thereof, wherein the substituents are as described.

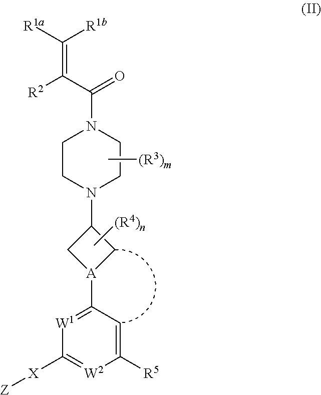

### CROSS-REFERENCE TO RELATED APPLICATIONS

This application claims the benefit of priority to U.S. Provisional Patent Application No. 63/413,548, filed Oct. 5, 2022, which is hereby incorporated by reference in its entirety, and for all purposes as if fully set forth herein.

### FIELD

The present disclosure relates generally to compounds having activity as inhibitors of the G12C-mutant KRAS protein, pharmaceutical compositions comprising the compounds, and uses and methods of treating disorders such as cancer, including but not limited to lung, pancreatic and colorectal cancer.

### BACKGROUND

The KRAS oncoprotein is a G-protein that couples extracellular mitogenic signaling to intracellular, pro-proliferative responses. KRAS functions as a molecular “on/off” switch, alternating between an inactive GDP-bound state and an active GTP-bound state. Transition between these states is facilitated by guanine nucleotide-exchange factors. Mitogen stimulation can induce GTP binding, which results in a conformational change that enables KRAS to interact with downstream effector proteins, leading to cellular proliferation. In normal cells, the pro-proliferative signaling is regulated by the action of GTPase-activating proteins (GAPs), which return KRAS to its GDP-bound, non-proliferative state. Mutations in KRAS impair the regulated cycling of KRAS between these GDP- and GTP-bound states, leading to the accumulation of the GTP-bound active state and dysregulated cellular proliferation. See Simanshu et al., Cell 2017, 170, 17-33.

Attempts to develop inhibitors of mutated KRAS proteins have historically been thwarted by the picomolar affinity with which KRAS binds to GDP and GTP, as well as the absence of druggable pockets on the surface of the protein. See Cox et al., Nat. Rev. Drug Discov. 2014, 13, 828-851. Covalent inhibitors of the G12C mutant of KRAS (“KRAS^{G12C}”) have been identified. These inhibitors can bind to a previously unrecognized allosteric pocket on GDP-KRAS^{G12C}, preventing its subsequent activation. See O'Bryan, J. P. Pharmacol. Res. 2019, 139, 503-511 and Ostrem et al., Nature 2013, 503, 548-551. This discovery brought about significant new efforts in KRAS inhibitor research, recently culminating in the entry of KRAS inhibitors into human clinical trials. While some progress has been made, the need for further KRAS^{G12C} inhibitors for the treatment of disorders, such as cancer, remains.

### SUMMARY

One aspect of the disclosure provides a compound of Formula (I):

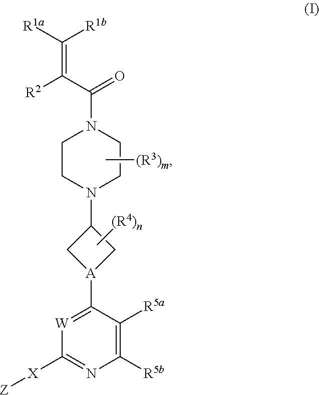

or a pharmaceutically acceptable salt thereof,
wherein:
m is 0, 1, 2, 3, or 4;
n is 1 or 2;
is 0, 1, 2, 3, or 4;
A is N, CH, C-halo, C—CN, C₁₋₃alkyl, C₁₋₃haloalkyl, C—C₀₋₃alkyleneOH, or C—C₀₋₃alkylene-C₁₋₄alkoxy;
W is CH, C-halo, C—CN, C—C₁₋₃alkyl, C—C₁₋₃haloalkyl, C—C₀₋₃alkyleneOH, or C—C₀₋₃alkylene-C₁₋₄alkoxy;
X is

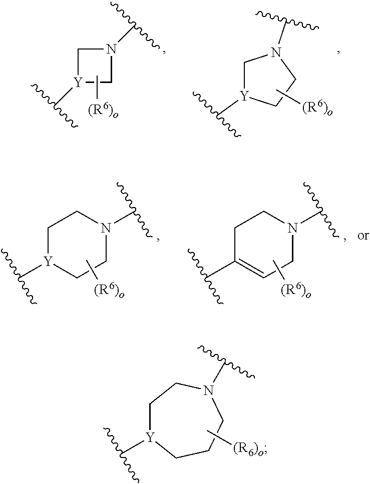

Y is N, C—H. C-halo, C—CN, C—C₁₋₃alkyl, C—C₁₋₃haloalkyl, C—C₀₋₂alkyleneOH, or C—C₀₋₃alkylene-C₁₋₄alkoxy;
Z is phenyl, heteroaryl comprising 5 or 6 total ring atoms and 1-3 heteroatoms selected from N, O, and S, or a bicyclic ring comprising a heteroaryl ring having 5 or 6 total ring atoms and 1-3 heteroatoms selected from N, O, and S fused to a cycloalkyl ring having 5 or 6 total ring atoms or a heterocycloalkyl ring having 5 or 6 total ring atoms and 1 or 2 heteroatoms selected from N, O, and S; wherein each of the phenyl, heteroaryl, and bicyclic rings is optionally substituted with 1-4 substituents;
each of R^{1a}, R^{1b}, and R² independently is H, D, halo, C₁₋₄alkyl, C₁₋₄haloalkyl, C₁₋₂alkylene-OH, C₀₋₂alkylene-C₁₋₄alkoxy, C₀₋₂alkylene-C₁₋₄haloalkoxy, C₀₋₂alkylene-CN, C₀₋₂alkylene-N(R^{N1})₂, C₁₋₂alkylene-heterocycloalkyl having 3-6 total ring atoms and 1 or 2 heteroatoms selected from N, O, and S, or R^{1b} and R², together with the carbon atoms to which they are attached, from a

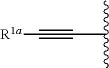

group;
each R³ independently is C₁₋₃alkyl, C₁₋₃haloalkyl, C₀₋₃alkyleneCN, C₀₋₃alkyleneOH, C₀₋₃alkylene-C₁₋₃alkoxy, oxo, spiro-cycloalkyl having 3-7 total ring atoms, spiro-heterocycloalkyl having 3-7 total ring atoms and 1 or 2 heteroatoms selected from N, O and S, or two adjacent R³, together with the atoms to which they are attached, form a fused cycloalkyl ring having 3-7 total ring atoms;
one R⁴ and R^{5a}, together with the atoms to which they are attached, form an optionally substituted ring having 6-10 total ring atoms and 0, 1, or 2 heteroatoms selected from N, O, and S, wherein the ring is saturated or unsaturated;
when n is 2, the other R⁴ is C₁₋₃alkyl, C₁₋₃haloalkyl, C₀₋₃alkyleneCN, C₁₋₂alkyleneOH, C₁₋₃alkylene-C₁₋₃alkoxy, oxo, spiro-cycloalkyl having 3-7 total ring atoms, or spiro-heterocycloalkyl having 4-7 total ring atoms and 1 or 2 heteroatoms selected from N, O and S;
R^{5b} is C₁₋₃haloalkyl, C₁₋₄alkyl, C₂₋₃alkenyl, C₂₋₃alkynyl, halo, C₁₋₃alkoxy, C₁₋₃thioalkoxy, cycloalkyl having 3-7 total ring atoms, cycloalkenyl having 5-7 total ring atoms, heterocycloalkyl having 3-7 total ring atoms and 1-3 heteroatoms selected from N, O, and S, or heterocycloalkenyl having 5-7 total ring atoms and 1-3 heteroatoms selected from N, O, and S, wherein each of foregoing is independently optionally substituted with 1-3 substituents;
each R⁶ independently is halo, CN, oxo, C₁₋₃alkyl, C₁₋₃haloalkyl, C₀₋₃alkyleneOH, C₀₋₃alkylene-C₁₋₃alkoxy, deuterated C₀₋₃alkylene-C₁₋₃alkoxy, C₁₋₄alkylene-N(R^{N1})₂, spiro-cycloalkyl having 3-7 total ring atoms, spiro-heterocycloalkyl having 4-7 total ring atoms and 1 or 2 heteroatoms selected from N, O and S; or two adjacent R⁶, together with the atoms to which they are attached, form a fused cycloalkyl ring having 3-7 total ring atoms; or Y and an adjacent R⁶, together with the atoms to which they are attached, form a fused cycloalkyl ring having 3-7 total ring atoms; wherein the fused cycloalkyl ring of any of the foregoing is optionally substituted with 1 or 2 substituents; or
two non-adjacent R⁶ join together to form a C₁₋₃alkylene bridge or a C₁₋₃ether bridge; and
each R^{N1} independently is H or C₁₋₄alkyl.

In some cases,

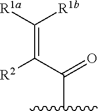

is

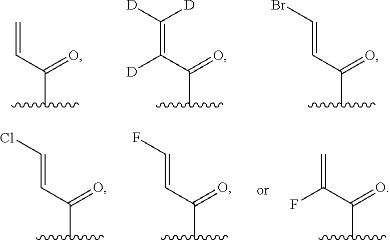

In some cases,

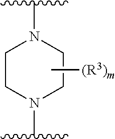

is

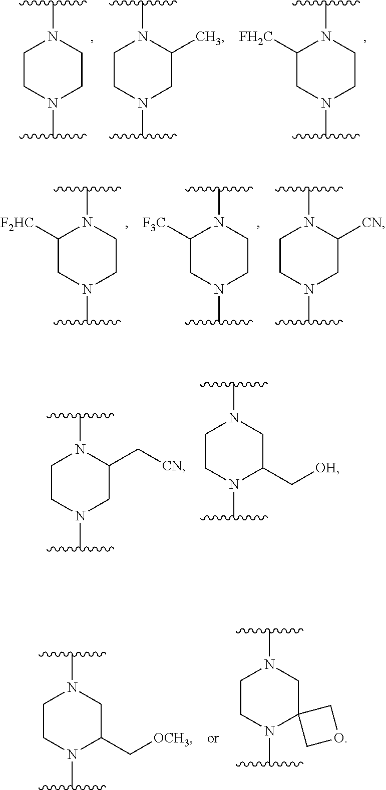

In some cases, A is N. In some cases, n is 1. In some cases, n is 2. In some cases, R⁴ is CH₃, CH₂CH₃, CH₂CH₂CH₃, CH(CH₃)₂, CF₃, CHF₂, or CH₂F. In some cases, W is CH.

In some cases, one R⁴ and R^{5a}, together with the atoms to which they are attached, form an optionally substituted ring having 6-10 total ring atoms and 0, 1, or 2 heteroatoms selected from N, O, and S, wherein the ring is saturated. In some cases, one R⁴ and R^{5a}, together with the atoms to which they are attached, form an optionally substituted ring having 6-10 total ring atoms and 0, 1, or 2 heteroatoms selected from N, O, and S, wherein the ring is unsaturated. In some cases, the optionally substituted ring has 6 or 7 total ring atoms. In some cases, the optionally substituted ring has 0 heteroatoms. In some cases, the optionally substituted ring has 1 or 2 heteroatoms selected from N, O, and S. In some cases, the 1 or 2 heteroatoms are each O. In some cases, the 1 or 2 heteroatoms are each N. In some cases, the ring formed by one R⁴ and R^{5a}, together with the atoms to which they are attached, is unsubstituted. In some cases, the ring formed by one R⁴ and R^{5a}, together with the atoms to which they are attached, is substituted with 1 or 2 substituents selected from the group consisting of C₁₋₃alkyl, C₁₋₃haloalkyl, oxo, halo, CN, C₀₋₃alkyleneOH, C₀₋₃alkylene-C₁₋₃alkoxy, cycloalkyl having 3-7 total ring atoms, cycloalkenyl having 5-7 total ring atoms, heterocycloalkyl having 4-7 total ring atoms and 1-3 heteroatoms selected from N, O, and S, heterocycloalkenyl having 5-7 total ring atoms and 1-3 heteroatoms selected from N, O, and S, and phenyl. In some cases, is

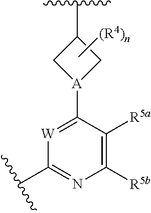

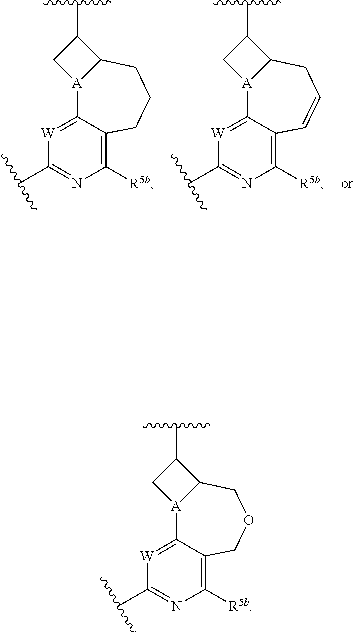

In some cases, R^{5b} is CF₃, CF₂H, CFH₂, or CF₂CH₃.

In some cases, X is

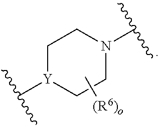

In some cases, Y is C—H. In some cases, o is 0. In some

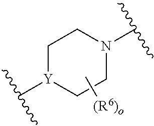

cases, o is 1. In some cases, R⁶ is CH₃, CH₂F, CHF₂, or CF₃. In some cases,

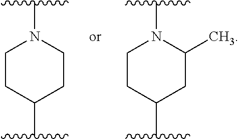 [SMILES: *C1CCN(*)CC1 ; *C1CCN(*)C(C)C1]

In some cases, Z is heteroaryl comprising 5 or 6 total ring atoms and 1-3 heteroatoms selected from N, O, and S, wherein the heteroaryl is optionally substituted with 1-4 substituents. In some cases, the heteroaryl is pyrrolyl, furanyl, thiophenyl, pyrazolyl, imidazolyl, thiazolyl, isothiazolyl, oxazolyl, isoxazolyl, triazolyl, thiadiazolyl, oxadiazolyl, pyridyl, pyridazinyl, pyrimidinyl, pyrazinyl, or triazinyl. In some cases, the heteroaryl is pyrazolyl or pyridyl. In some cases, the heteroaryl is substituted with 1-4 substituents, each of which independently is selected from the group consisting of halo, CN, C₁₋₆alkyl, C₁₋₃haloalkyl, C₂₋₆alkenyl, C₂₋₆ haloalkenyl, C₀₋₆alkylene-OH, C₀₋₃alkylene-C₁₋₃alkoxy, C₀₋₃alkylene-N(R^{N1})₂ wherein each R^{N1} independently is H or C₁₋₃alkyl, C₀₋₂alkylene-cycloalkyl having 3-6 total ring atoms, C₀₋₂alkylene-heterocycloalkyl having 3-6 total ring atoms and 1-3 heteroatoms selected from N, O, and S, and C₀₋₂alkylene-phenyl; wherein each of the alkyl, alkenyl, C₀₋₆alkylene-C₁₋₃alkoxy, cycloalkyl, heterocycloalkyl, and phenyl substituents independently is optionally substituted with 1-3 substituents independently selected from deuterium, halo, OH, CH₃, OCH₃, and OCD₃. In some cases, each of the 1-4 substituents independently is selected from the group consisting of Cl, F, CN, CH₃, CD₃, CH₂CH₃, CH(CH₃)₂, CF₃, CHF₂, CH₂F, CH₂CHF₂, CH₂CH₂F, CH(CH₂F)₂, CH(CH₃)CH₂F, CH(CH₃)CHF₂, C(═CH₂)CH₂F, OH, CH₂OH, CH₂CH₂OH, CH(CH₃)CH₂OH, C(CH₃)₂OH, C(CH₃)₂CH₂OH, CH₂C(CH₃)₂OH, OCH₃, OCD₃, CH₂OCH₃, CH₂OCD₃, CH₂CH₂OCH₃, CHFCH₂OCH₃, CF₂CH₂OCH₃, CH₂CH₂OCD₃, CH₂CH₂OCH₂CH₂CH₂CH₂CH₂OCH₃, CH₂CH₂CH₂OCD₃, CH(CH₃)CH₂OCH₃, CH(CH₃)CH₂OCD₃,C(CH₃)₂CH₂OCH₃, C(CH₃)₂CH₂OCD₃, CH₂CH(CH₃)OCH₃, CH₂CH(CH₃)OCD₃, CH₂C(CH₃)₂OCH₃, CH₂C(CH₃)₂OCD₃, NH₂, CH₂NH₂, CH₂NHCH₃, CH₂N(CH₃)₂, CH₂CH₂NH₂, CH₂CH₂NHCH₃, CH₂CH₂N(CH₃)₂,

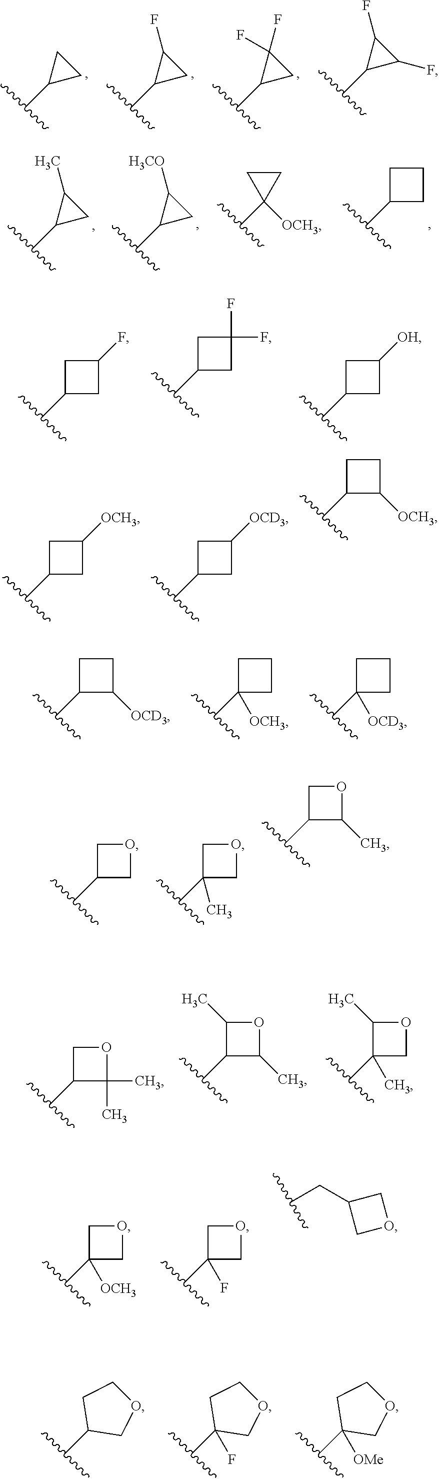

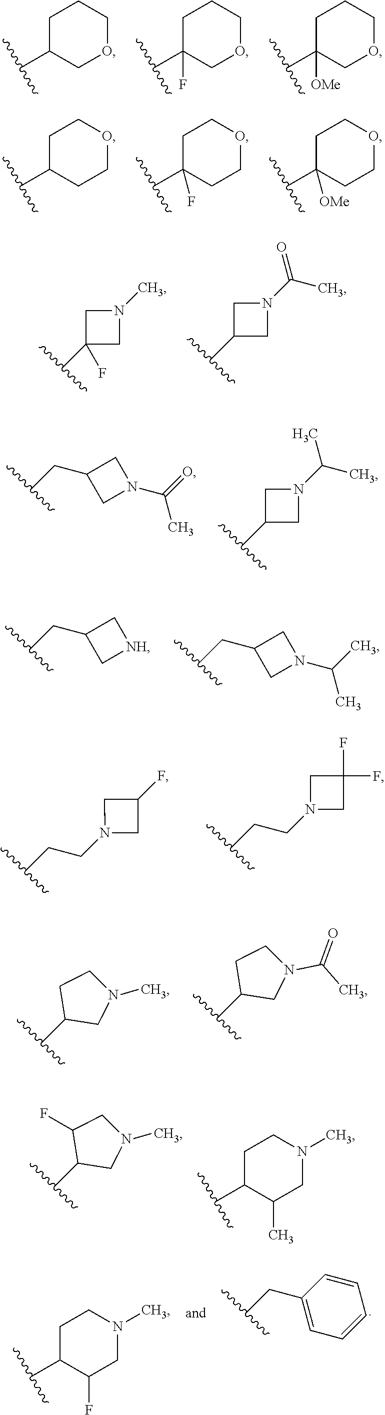

In some cases, each of the 1-4 substituents independently is CH₃, CH₂CH₂OCH₃, CH₂CH₂OCD₃,

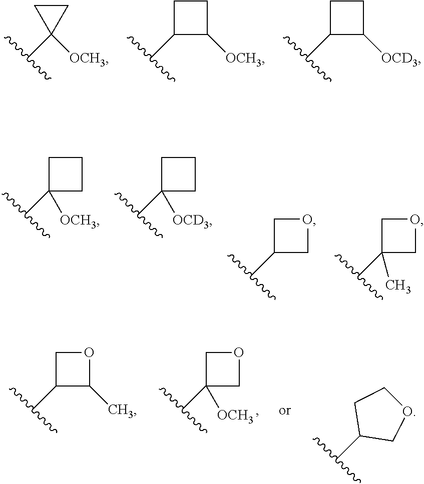

In some cases, Z is

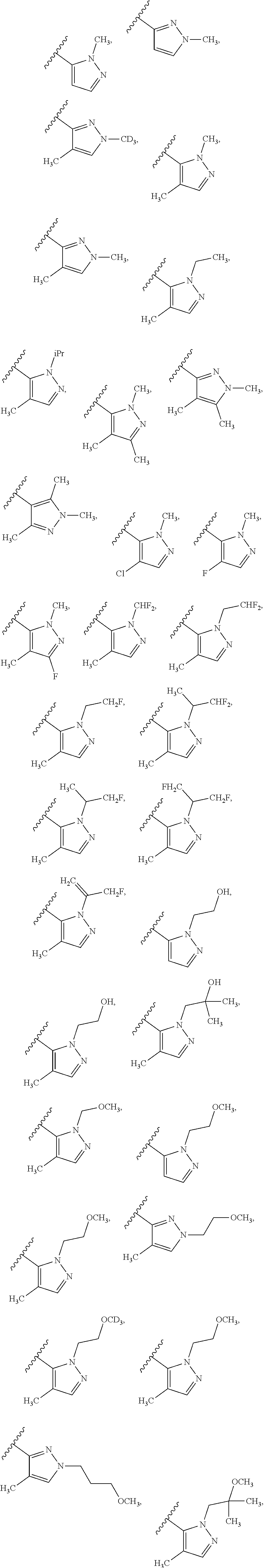

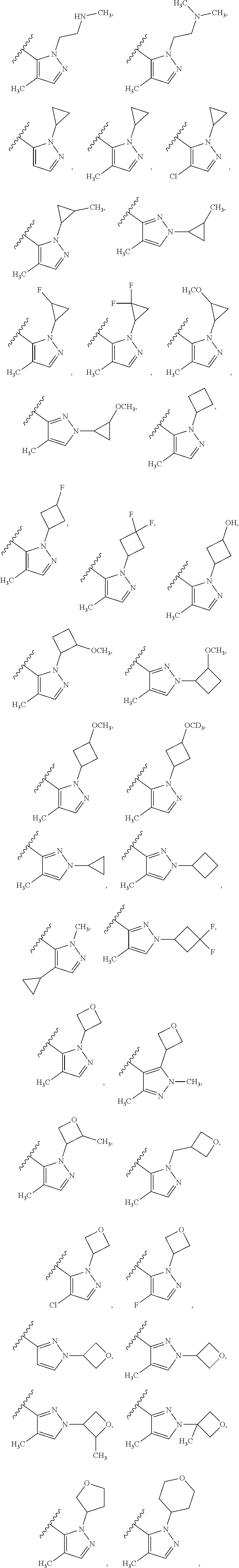

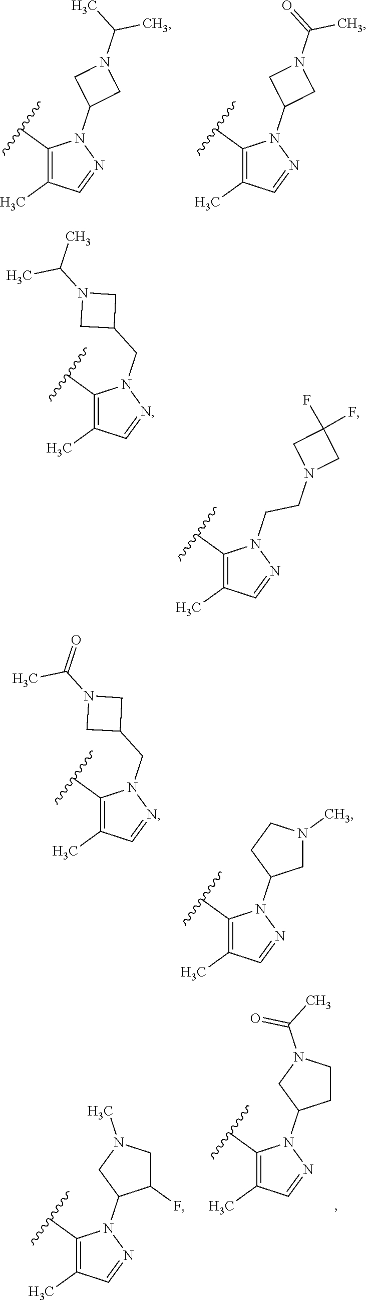

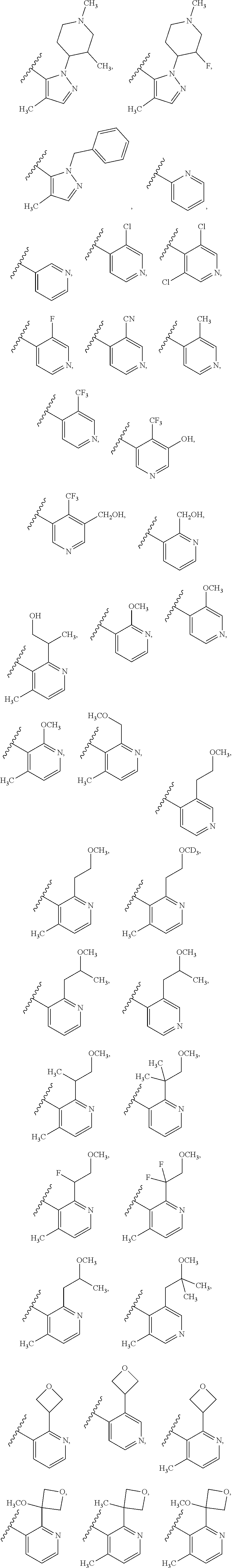

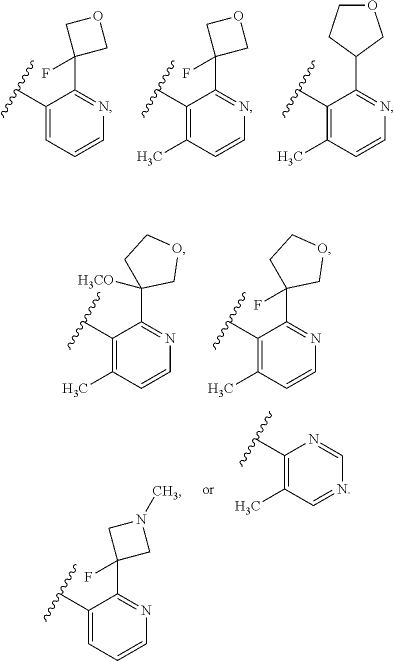

In some cases, Z is

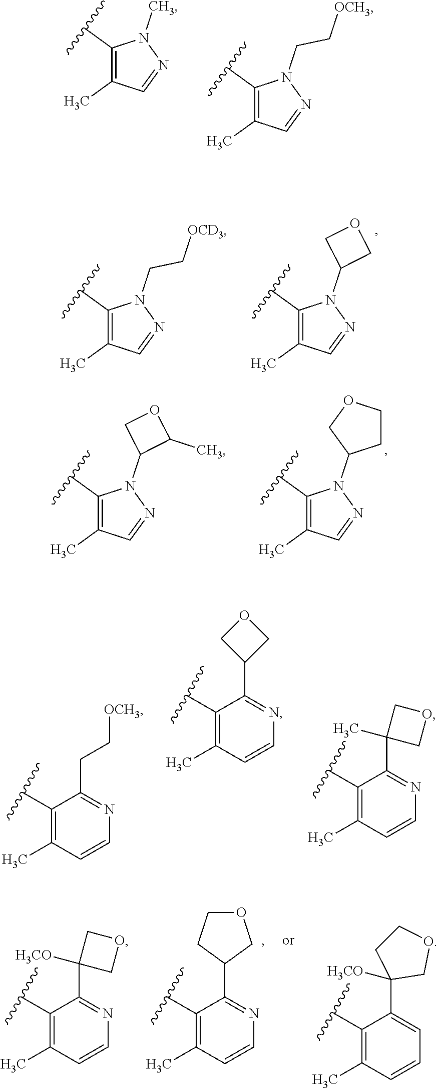

In some cases,

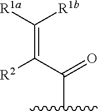

is

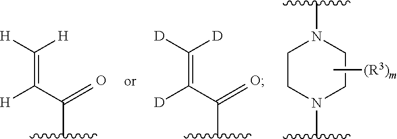

is

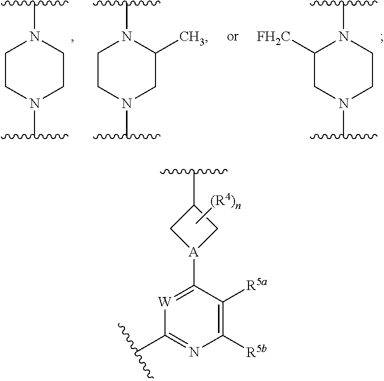

is

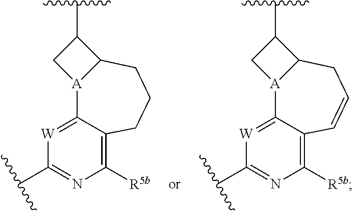

### X is

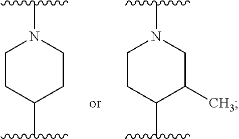 [SMILES: *C1CCN(*)CC1 ; *C1CCN(*)CC1C]

and Z is pyrazolyl or pyridyl, each of which is optionally substituted with 1-4 substituents. In some cases, the 1-4 substituents of Z independently is CH₃, CH₂CH₂OCH₃, CH₂CH₂OCD₃,

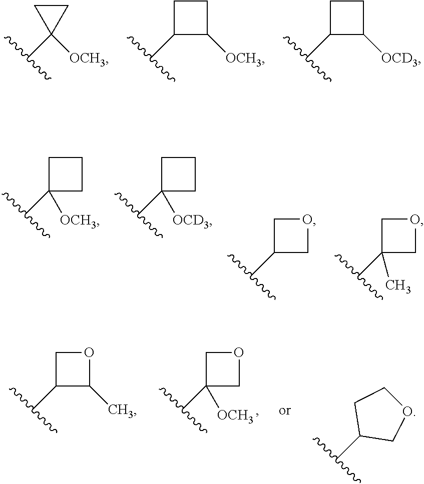

In some cases, Z is substituted with 2 substituents.

In some cases, one substituent of Z is CH₃. In some cases, one substituent of Z is CH₃, and the other substituent of Z is CH₃, CH₂CH₂OCH₃,

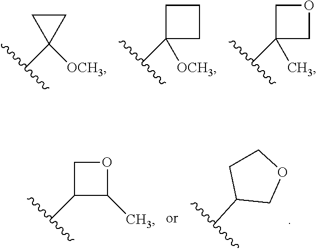 [SMILES: *C1(OC)CC1 ; *C1(OC)CCC1 ; *C1(C)COC1 ; *C1COC1C ; "or" ; *C1CCOC1]

In some cases, Z is

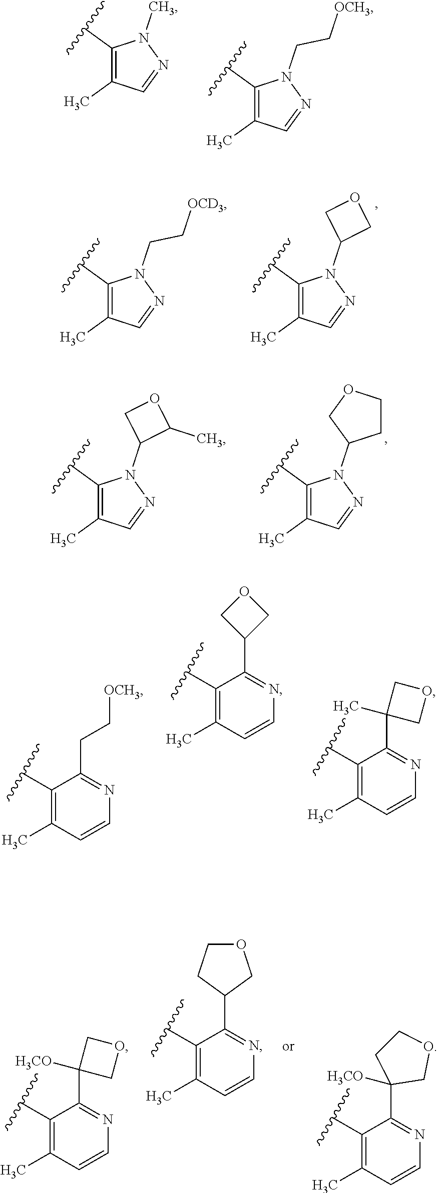

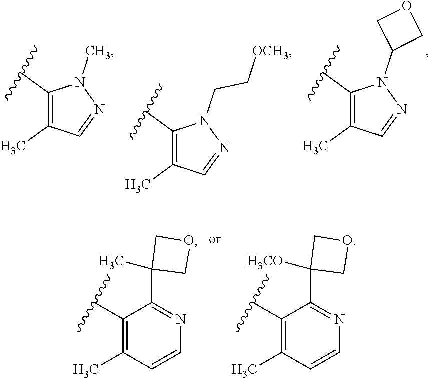

In some cases, the compound of Formula (I) is a compound having a structure:

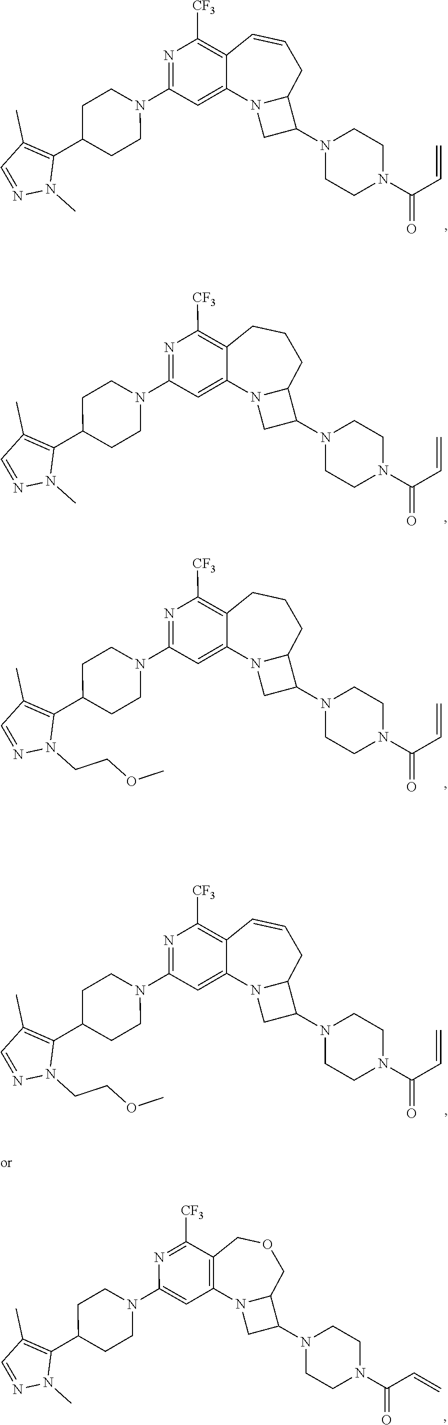 [SMILES: C=CC(=O)N1CCN(C2CN3c4cc(N5CCC(c6c(C)cnn6C)CC5)nc(C(F)(F)F)c4C=CCC23)CC1 ; C=CC(=O)N1CCN(C2CN3c4cc(N5CCC(c6c(C)cnn6C)CC5)nc(C(F)(F)F)c4CCCC23)CC1 ; C=CC(=O)N1CCN(C2CN3c4cc(N5CCC(c6c(C)cnn6CCOC)CC5)nc(C(F)(F)F)c4CCCC23)CC1 ; C=CC(=O)N1CCN(C2CN3c4cc(N5CCC(c6c(C)cnn6CCOC)CC5)nc(C(F)(F)F)c4C=CCC23)CC1 ; C=CC(=O)N1CCN(C2CN3c4cc(N5CCC(c6c(C)cnn6C)CC5)nc(C(F)(F)F)c4COCC23)CC1]

or a pharmaceutically acceptable salt thereof. In some cases, the compound of Formula (I) is a compound having a structure:

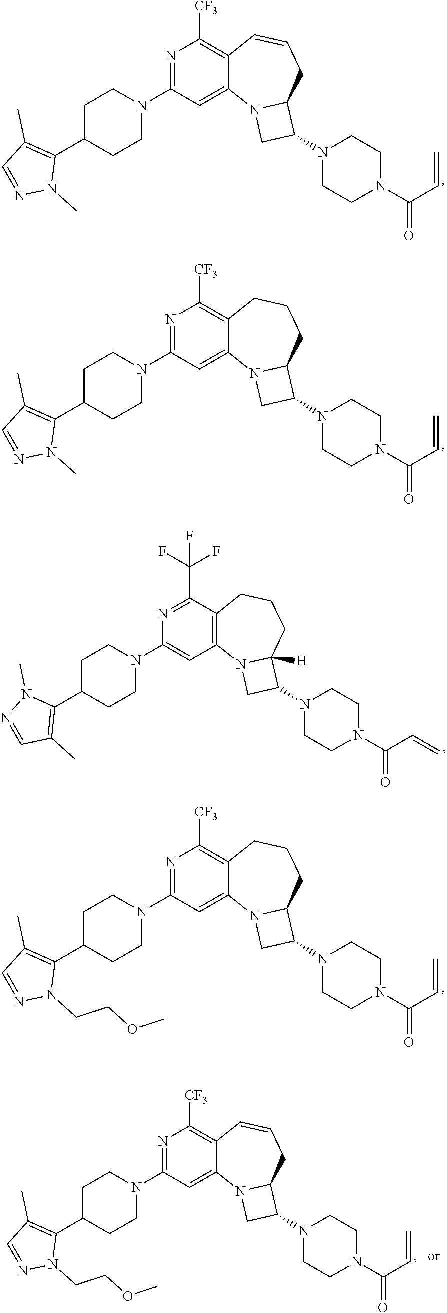

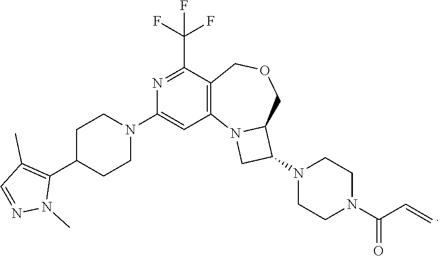 [SMILES: C=CC(=O)N1CCN([C@@H]2CN3c4cc(N5CCC(c6c(C)cnn6C)CC5)nc(C(F)(F)F)c4COC[C@@H]23)CC1]

or a pharmaceutically acceptable salt thereof.

Another aspect of the disclosure provides a compound of Formula (II):

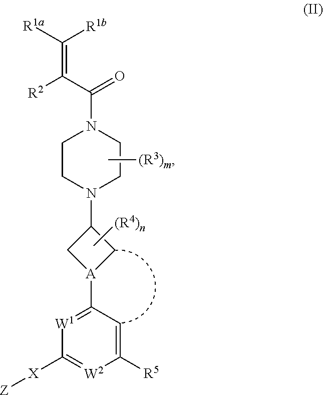

or a pharmaceutically acceptable salt thereof, wherein:

m is 0, 1, 2, 3, or 4;
n is 0, 1, or 2;
A is N, CH, C-halo, C—CN, C—C₁₋₃alkyl, C—C₁₋₃haloalkyl, C—C₀₋₂alkyleneOH, or C—C₀₋₃alkylene-C₁₋₄alkoxy;
each of W¹ and W² independently is N, CH, C-halo, C—CN, C—C₁₋₃alkyl, C—C₂₋₃alkenyl, C—C₂₋₃alkynyl, C—C₁₋₃haloalkyl, C—C₀₋₂alkyleneOH, or C—C₀₋₃alkylene-C₁₋₄alkoxy, wherein each of the alkenyl and alkynyl is unsubstituted or substituted with 1-3 substituents and each substituent independently is halo, C₁₋₃haloalkyl, C₀₋₃alkyleneOH, or C₀₋₃alkyleneC₁₋₄alkoxy;
X is heterocycloalkyl or heterocycloalkenyl, each having 4-7 total ring atoms and 1-3 heteroatoms selected from N, O, and S, wherein each of the heterocycloalkyl and heterocycloalkenyl is unsubstituted or substituted with 1-3 substituents, and each substituent independently is halo, C₁₋₃alkyl, C₁₋₃haloalkyl, C₀₋₂alkyleneOH, C₀₋₂alkyleneC₁₋₃alkoxy, or C₀₋₂alkyleneCN;
Z is phenyl, heteroaryl comprising 5 or 6 total ring atoms and 1-3 heteroatoms selected from N, O, and S, or a bicyclic ring comprising a heteroaryl having 5 or 6 total ring atoms and 1-3 heteroatoms selected from N, O, and S fused to C₅₋₆cycloalkyl or a heterocycloalkyl ring having 5 or 6 total ring atoms and 1 or 2 heteroatoms selected from N, O, and S;
wherein each of the phenyl, heteroaryl, and bicyclic ring is unsubstituted or substituted with 1-4 substituents, and each substituent independently is halo, CN, C₁₋₆alkyl, C₃₋₆haloalkyl, C₂₋₆alkenyl, C₂₋₆haloalkenyl, C₀₋₃alkylene-OH, C₀₋₆alkylene-C₁₋₃alkoxy, C₀₋₆alkylene-N(R^{N1})₂, C₀₋₂alkylene-C₃₋₆cycloalkyl, C₀₋₂alkylene-heterocycloalkyl having 3-6 total ring atoms and 1-3 heteroatoms selected from N, O, and S, or C₀₋₂alkylene-phenyl;
wherein each of the C₁₋₆alkyl, C₂₋₆alkenyl, C₀₋₆alkylene-C₁₋₃alkoxy, cycloalkyl, heterocycloalkyl, and phenyl substituents independently is unsubstituted or substituted with 1-3 further substituents, and each further substituent independently is D, halo, C₁₋₃alkyl, C₁₋₃haloalkyl, C₁₋₂alkyleneOH, C₁₋₂alkylene-C₁₋₃alkoxy, C₁₋₃deuterated alkoxy, N(R^{N1})₂, (C═O) C₁₋₃alkyl, C₃₋₅cycloalkyl, or heterocycloalkyl having 3-5 total ring atoms and 1 or 2 heteroatoms selected from N, O, and S; or two geminal further substituents, together with the atom to which they are attached, form spiro-C₃₋₅cycloalkyl or spiro-heterocycloalkyl having 3-5 total ring atoms and 1 or 2 heteroatoms selected from N, O, and S; or two vicinal further substituents, together with the atoms to which they are attached, form fused-C₃₋₅cycloalkyl or fused-heterocycloalkyl having 3-5 total ring atoms and 1 or 2 heteroatoms selected from N, O, and S;
wherein each of the foregoing cycloalkyl and heterocycloalkyl further substituents independently is unsubstituted or substituted with 1 or 2 substituents, and each substituent independently is halo or C₁₋₃alkyl;
[symbol] is C₂₋₆alkylene, C₃₋₆alkenylene, heteroalkylene having 2-6 total atoms and 1-3 heteroatoms selected from N, O, and S, or heteroalkenylene having 3-6 total atoms and 1 or 2 heteroatoms selected from N, O, and S, wherein [symbol] is unsubstituted or substituted with 1-4 substituents, and each substituent independently is C₁₋₃alkyl, C₁₋₃haloalkyl, C₂₋₃alkenyl, halo, CN, C₀₋₃alkyleneOH, C₀₋₃alkylene-C₁₋₃alkoxy, C₃₋₅cycloalkyl, C₄₋₅cycloalkenyl, heterocycloalkyl having 4 or 5 total ring atoms and 1-3 heteroatoms selected from N, O, and S, heterocycloalkenyl having 4 or 5 total ring atoms and 1-3 heteroatoms selected from N, O, and S, or phenyl; or two geminal substituents, together with the atom to which they are attached, form oxo, =CH₂, spiro-C₃₋₅cycloalkyl, spiro-C₄₋₅cycloalkenyl, spiro-heterocycloalkyl having 3-5 total ring atoms and 1 or 2 heteroatoms selected from N, O and S, or spiro-heterocycloalkenyl having 4 or 5 total ring atoms and 1 or 2 heteroatoms selected from N, O and S; or two vicinal substituents, together with the atoms to which they are attached, form fused-C₃₋₅cycloalkyl, fused-C₄₋₅cycloalkenyl, fused-heterocycloalkyl having 3-5 total ring atoms and 1 or 2 heteroatoms selected from N, O and S or fused-heterocycloalkenyl having 4 or 5 total ring atoms and 1 or 2 heteroatoms selected from N, O and S;
each of R^{1a}, R^{1b}, and R² independently is H, D, halo, C₁₋₄alkyl, C₁₋₄haloalkyl, C₁₋₂alkylene-OH, C₀₋₂alkylene-C₁₋₄alkoxy, C₀₋₂alkylene-Cy-haloalkoxy, C₀₋₂alkylene-CN, C₀₋₂alkylene-N(R^{N1})₂, C₁₋₂alkylene-heterocycloalkyl having 3-6 total ring atoms and 1 or 2 heteroatoms selected from N, O, and S, or R^{1b} and R², together with the carbon

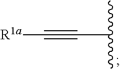

atoms to which they are attached, form

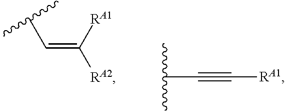

each R³ independently is C₁₋₃alkyl, C₁₋₄haloalkyl, C₀₋₃alkyleneCN, C₀₋₃alkyleneOH, or C₀₋₆alkylene-C₁₋₃alkoxy; or two geminal R³, together with the atom to which they are attached, form oxo, spiro-C₁₋₃-cycloalkyl, spiro-C₄₋₇cycloalkenyl, spiro-heterocycloalkyl having 3-7 total ring atoms and 1 or 2 heteroatoms selected from N, O, and S, or spiro-heterocycloalkenyl having 4-7 total ring atoms and 1 or 2 heteroatoms selected from N, O, and S; or two vicinal R³, together with the atoms to which they are attached, form fused-C₃₋₇cycloalkyl, fused-C₄₋₇cycloalkenyl, fused-heterocycloalkyl having 3-7 total ring atoms and 1 or 2 heteroatoms selected from N, O, and S, or fused-heterocycloalkenyl having 4-7 total

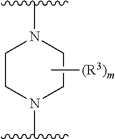

ring atoms and 1 or 2 heteroatoms selected from N, O, and S; or is deuterated;

each R⁴ independently is C₁₋₃alkyl, C₁₋₃haloalkyl, C₀₋₃alkyleneCN, C₁₋₃alkyleneOH, or C₁₋₃alkylene-C₁₋₃alkoxy; or two geminal R⁴, together with the atom to which they are attached, form oxo, spiro-C₃₋₇cycloalkyl, or spiro-heterocycloalkyl having 4-7 total ring atoms and 1 or 2 heteroatoms selected from N, O and S;

R⁵ is halo, C₁₋₃haloalkyl, C₁₋₆alkyl, C₂₋₄alkenyl, C₂₋₄alkynyl, C₁₋₃alkoxy, C₁₋₅thioalkyl, C₃₋₇cycloalkyl, C₅₋₇cycloalkenyl, heterocycloalkyl having 3-7 total ring atoms and 1-3 heteroatoms selected from N, O, and S, or heterocycloalkenyl having 5-7 total ring atoms and 1-3 heteroatoms selected from N, O, and S, wherein each of the foregoing independently is unsubstituted or substituted with 1-3 substituents, and each substituent independently is C₁₋₃haloalkyl, C₀₋₆alkylene-OH, C₀₋₃alkylene-C₁₋₃alkoxy, C₂₋₇cycloalkyl, C₅₋₇cycloalkenyl, heterocycloalkyl having 3-7 total ring atoms and 1-3 heteroatoms selected from N, O, and S, heterocycloalkenyl having 4-7 total ring atoms and 1-3 heteroatoms selected from N, O, and S, or phenyl;

each of R^{A1} and R^{A2} independently is H, C₁₋₃alkyl, C₁₋₄haloalkyl, or C₃₋₅cycloalkyl; and

each R^{N1} independently is H or C₁₋₄alkyl.

In some cases, at least one of R^{1a}, R^{1b}, and R² is H or D. In some cases, each of R^{1a}, R^{1b}, and R² independently is H or D. In some cases, two of R^{1a}, R^{1b}, and R² are H and one of R^{1a}, R^{1b}, and R² is halo, C₁₋₄alkyl, C₁₋₄haloalkyl, C₁₋₂alkylene-OH, C₀₋₂alkylene-C₁₋₃alkoxy, C₁₋₃alkylene-C₁₋₄haloalkoxy, C₀₋₂alkylene-CN, C₀₋₂alkylene-N(R^{N1})₂, or C₁₋₂alkylene-heterocycloalkyl having 3-6 total ring atoms

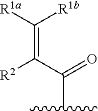

and 1 or 2 heteroatoms selected from N, O, and S. In some cases, is

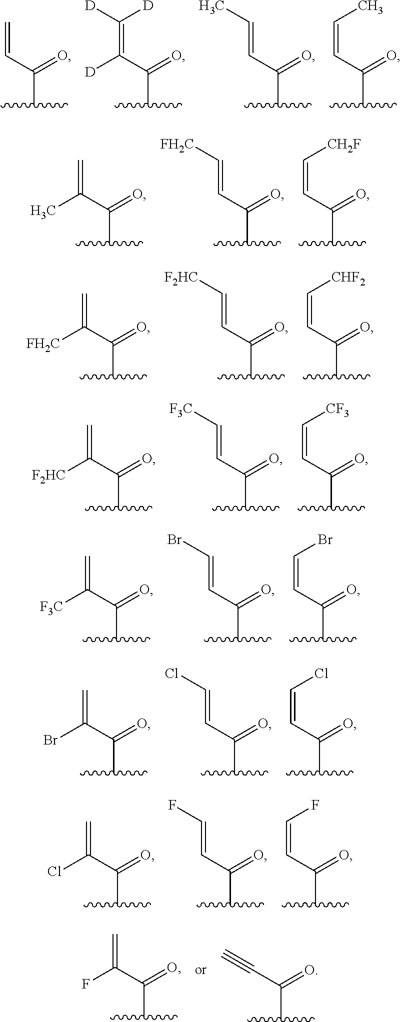

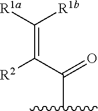

In some cases, IS

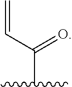 [SMILES: *C(=O)C=C]

In some cases, m is 0. In some cases, m is 1. In some cases, m is 2. In some cases, each R³ independently is CH₃, CH₂CH₃, CH₂F, CHF₂. CF₃, CN, CH₂CN, OH, CH₂OH, CH₂CH₂OH, OCH₃, CH₂OCH₃, or CH₂CH₂OCH₃; two geminal R³, together with the atom to which they are attached, form oxo, spiro-cyclopropyl, spiro-cyclobutyl, spiro-oxetanyl, or spiro-tetrahydrofuranyl; or two vicinal R³, together with the atoms to which they are attached, form fused-cyclopropyl or fused-cyclobutyl. In some cases, m is 0; or m is 1 and R³ is CH₃, CH₂F, CHF₂, CF₃, CN, CH₂CN, CH₂OH, or CH₂OCH₃; or m is 2 and two geminal R³, together with the atom to which they are attached, form spiro-oxetanyl.

In some cases, m is 0; or m is 1 and R³ is CH₃. In some cases,

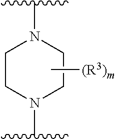

is

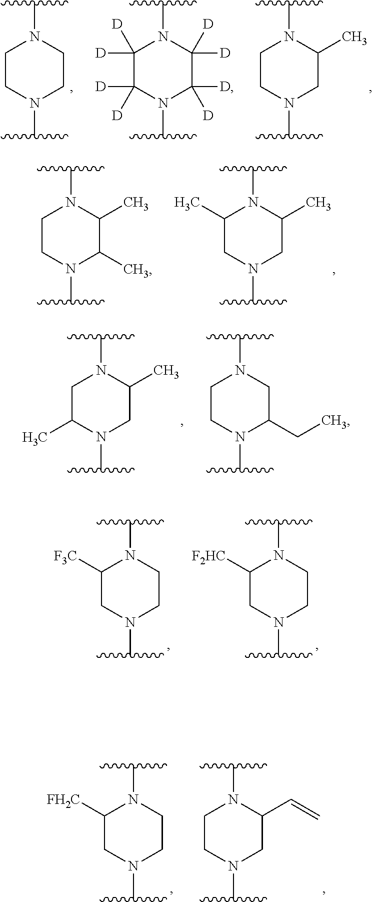

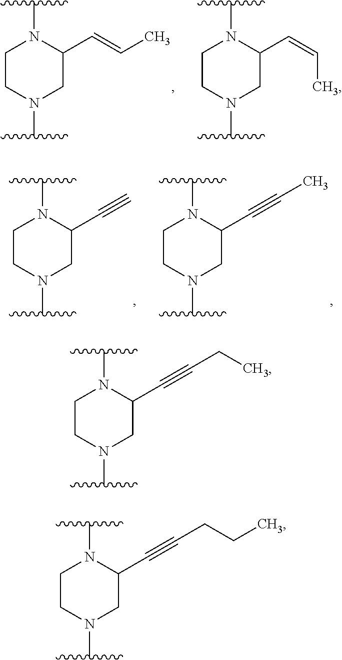

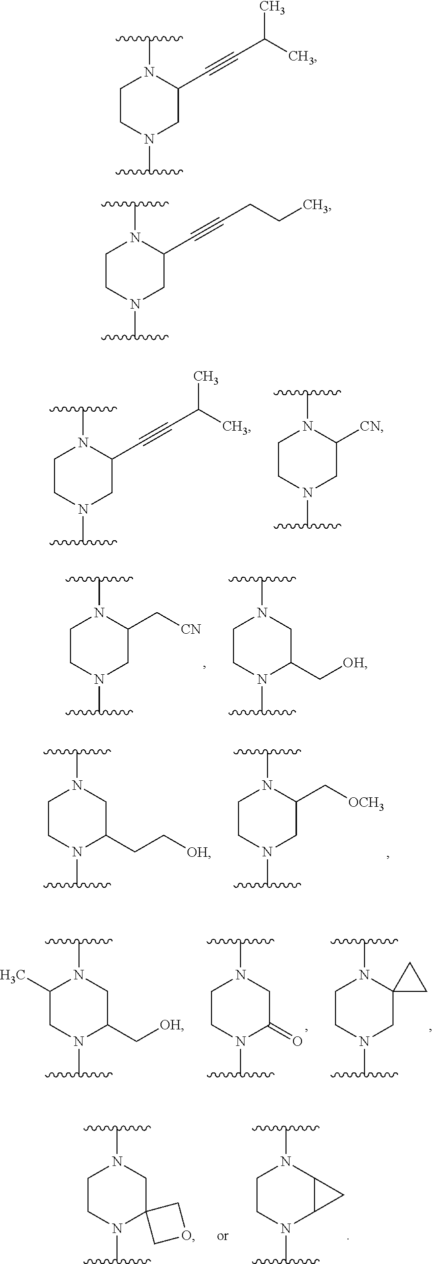

In some cases,

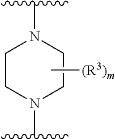

is

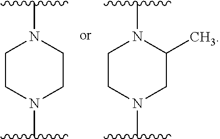

In some cases, A is N. In some cases, A is CH, C—F, C—Cl, C—CN, C—CH₃, C—CH₂F, C—CHF₂, C—CF₃, C—OH, C—CH₂OH, C—OCH₃, or C—CH₂OCH₃. In some cases, n is 0. In some cases, n is 1. In some cases, n is 2. In some cases, each R⁴ independently is CH₃, CH₂CH₃, CH₂CH₂CH₃, CH(CH₃)₂, CF₃, CHF₂, CH₂F, CN, CH₂CN, CH₂OH, CH₂CH₂OH, CH₂OCH₃, or CH₂CH₂OCH₃; or two geminal R⁴, together with the atom to which they are attached, form oxo, spiro-cyclopropyl, spiro-cyclobutyl, or spiro-oxetanyl. In some cases,

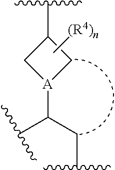

is

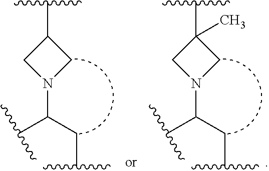

In some cases, [symbol] is C₂alkylene, C₃alkylene, C₃alkenylene, or heteroalkylene having 2-4 total atoms and 1 or 2 heteroatoms selected from N, O, and S. In some cases, [symbol] is unsubstituted. In some cases, [symbol] is substituted with CH₃, ═CH₂, oxo, Cl, F, OH, OCH₃

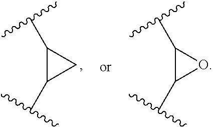 [SMILES: *C1CC1* ; *C1OC1*]

or a combination of the foregoing. In some cases, [symbol] is

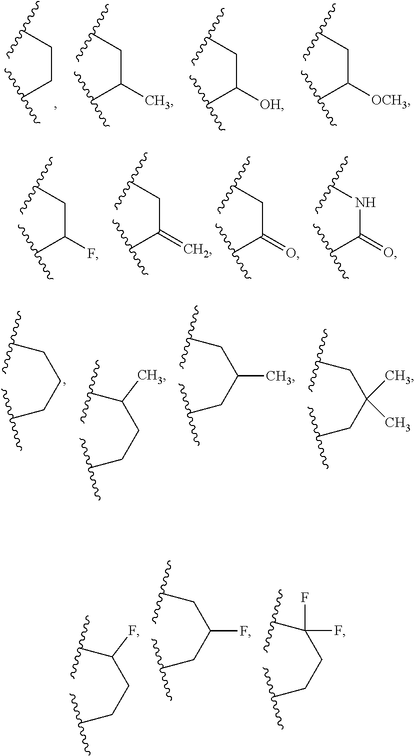

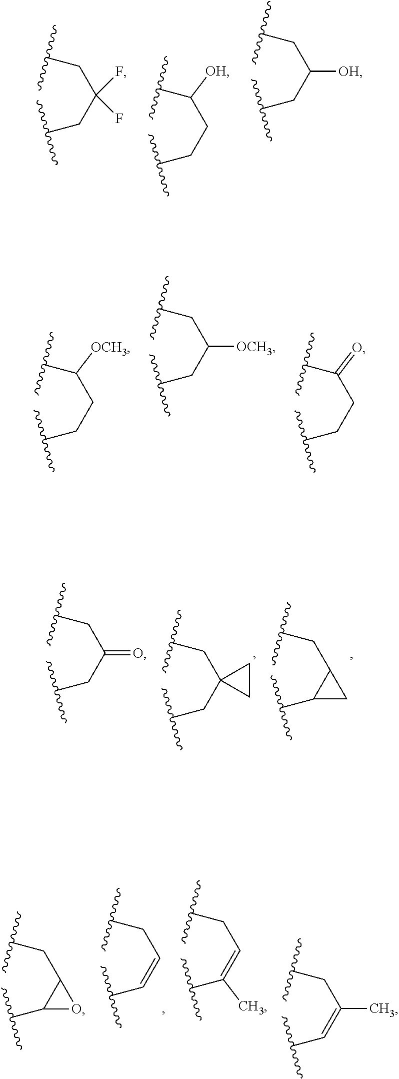

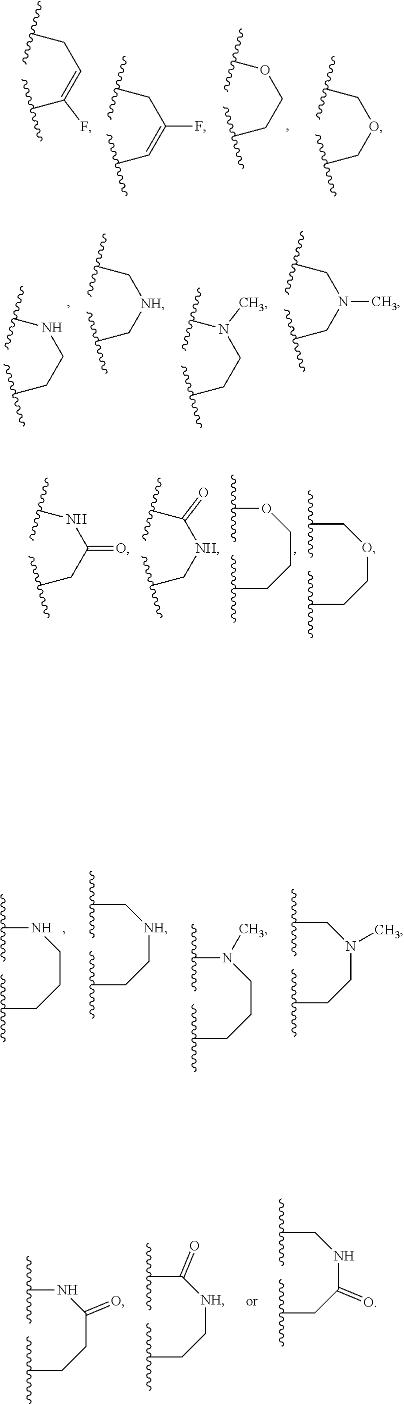

In some cases, [symbol] is

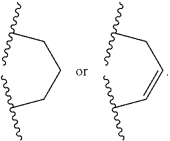 [SMILES: *CCC* ; */C=C\C*]

In some cases, [symbol] is

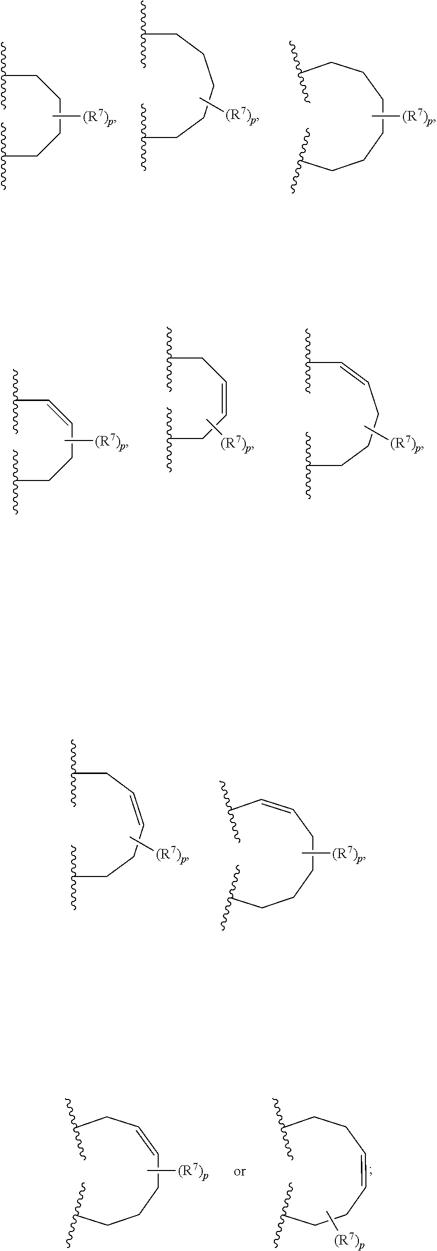

wherein p is 0, 1, 2, or 3, and each R⁷ independently is CH₃, Cl, F, OH, or OCH₃; or two geminal R⁷, together with the atom to which they are attached form oxo or =CH₂; or two vicinal R⁷, together with the atoms to which they are attached form

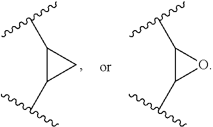 [SMILES: *C1CC1* ; *C1OC1*]

In some cases, W¹ is N. In some cases, WI is CH. In some cases, WI is C—F, C—Cl, C—CN. C—CH₃, C—CH₂CH₃, C—CH₂F, C—CHF₂, C—CF₃, C—CH═CH₂, C—C(OH)═CH₂, C—CH═CH(OH), C—CCH, C—OH, C₂CH₂OH, C—OCH₃, or C—CH₂OCH₃. In some cases, W² is N. In some cases, W² is CH. In some cases, W² is C—F, C—Cl, C—CN, C—CH₃, C—CH₂CH₃, C—CH₂F, C—CHF₂, C—CF₃, C—CH═CH₂, C—C(OH)═CH₂, C—CH═CH(OH), C—CCH, C—OH, C₂CH₂OH, C—OCH₃, or C—CH₂OCH₃. In some cases, W¹ is CH and W² is N. In some cases, R⁵ is C₁₋₅haloalkyl. In some cases, R⁵ is CF₃ or CF₂H. In some cases, R⁵ is CH₃, CH₂CH₃, CH₂CH₂CH₃, CH(CH₃)₂, CH═CH₂, CH═CHCH₃,

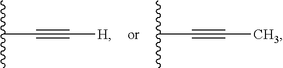

wherein each of the foregoing independently is unsubstituted or substituted with 1-3 substituents, and each substituent independently is C₁₋₃haloalkyl, C₀₋₆alkylene-OH, C₀₋₆alkylene-C₁₋₃alkoxy, C₃₋₇cycloalkyl, C₅₋₇cycloalkenyl, heterocycloalkyl having 3-7 total ring atoms and 1-3 heteroatoms selected from N, O, and S, heterocycloalkenyl having 4-7 total ring atoms and 1-3 heteroatoms selected from N, O, and S, or phenyl. In some cases, each substituent independently is CH₃, CF₃, CF₂H, CFH₂, OH, OCH₃, OCF₃, CH₂OH, CH₂OCH₃, cyclopropyl, cyclobutyl, or phenyl. In some cases. R⁵ is Br, Cl, F. OCH₃, SCH₃, CH₃, CH₂CH₃, CH₂CH₂CH₃, CH(CH₃)₂,

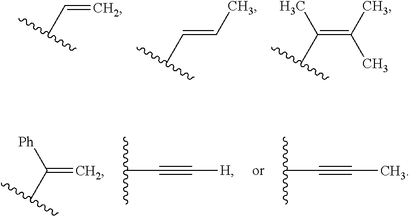

In some cases, W¹ is CH, W² is N, and R⁵ is CF₃, CF₂H, or CFH₂. In some cases,

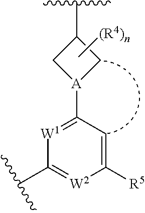

is

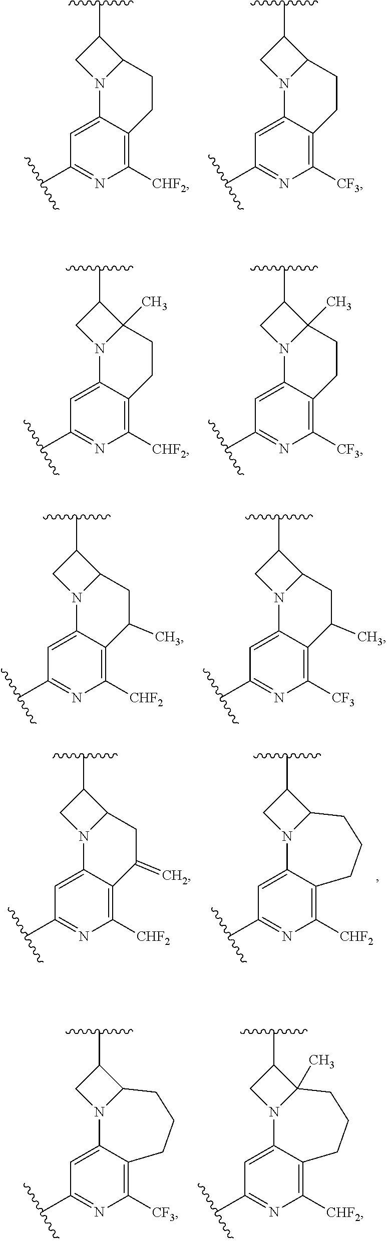

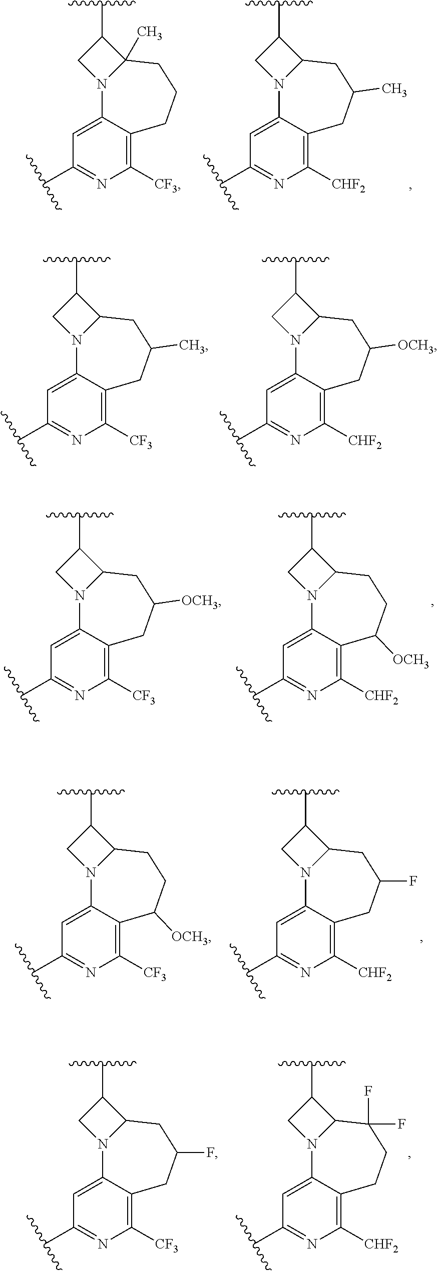

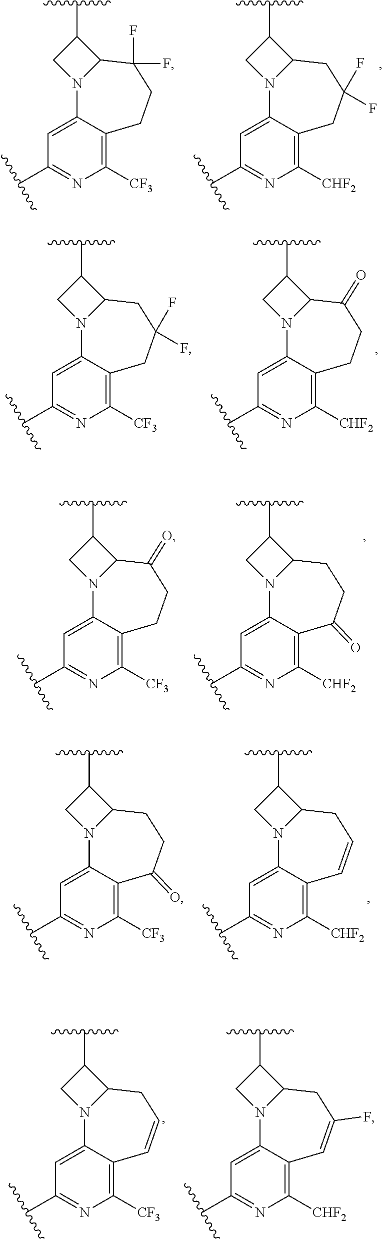

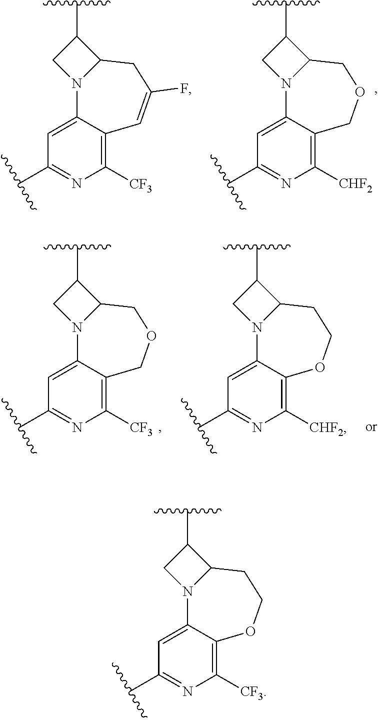 [SMILES: *c1cc2c(c(C(F)(F)F)n1)C=C(F)CC1C(*)CN21 ; *c1cc2c(c(C(F)F)n1)COCC1C(*)CN21 ; *c1cc2c(c(C(F)(F)F)n1)COCC1C(*)CN21 ; *c1cc2c(c(C(F)F)n1)OCCC1C(*)CN21 ; *c1cc2c(c(C(F)(F)F)n1)OCCC1C(*)CN21]

In some cases,

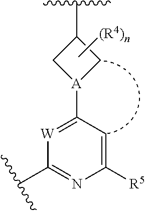

is

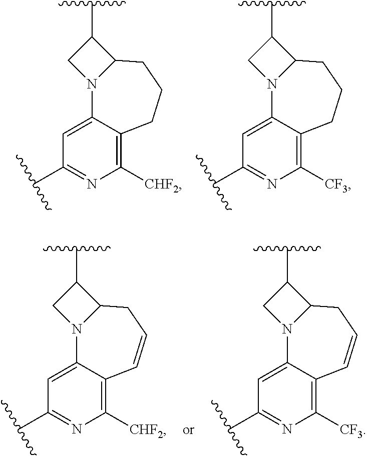 [SMILES: *c1cc2c(c(C(F)F)n1)CCCC1C(*)CN21 ; *c1cc2c(c(C(F)(F)F)n1)CCCC1C(*)CN21 ; *c1cc2c(c(C(F)F)n1)C=CCC1C(*)CN21 ; *c1cc2c(c(C(F)(F)F)n1)C=CCC1C(*)CN21]

In some cases, X is

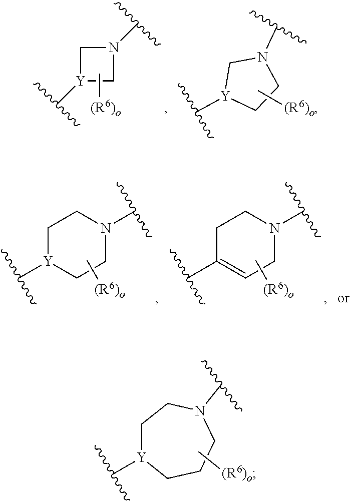

Y is N, C—H, C-halo, C—CN, C—C₁₋₃alkyl, C—C₁₋₃haloalkyl, C—C₀₋₂alkyleneOH, or C—C₀₋₃alkylene-C₁₋₄alkoxy; o is 0, 1, 2, 3, or 4; and each R⁶ independently is halo, CN, C₁₋₃alkyl, C₂₋₃alkenyl, C₁₋₃haloalkyl, C₀₋₃alkylene-OH, C₀₋₃alkylene-C₁₋₃alkoxy, deuterated C₀₋₃alkylene-C₁₋₃alkoxy, or C₁₋₄alkylene-N(R^{N1})₂; or two geminal R⁶, together with the atom to which they are attached, form oxo, =CH₂, spiro-C₅₋₇cycloalkyl, spiro-C₄₋₇cycloalkenyl, spiro-heterocycloalkyl having 4-7 total ring atoms and 1 or 2 heteroatoms selected from N, O and S, or spiro-heterocycloalkenyl having 4-7 total ring atoms and 1 or 2 heteroatoms selected from N, O and S; or two vicinal R⁶, together with the atoms to which they are attached, form fused-C₃₋₇cycloalkyl, fused-C₄₋₇cycloalkenyl, fused-heterocycloalkyl having 4-7 total ring atoms and 1-3 heteroatoms selected from N, O, and S, or fused-heterocycloalkenyl having 4-7 total ring atoms and 1 or 2 heteroatoms selected from N, O, and S; or two non-neighboring R⁶ join together to form a C₀₋₃alkylene bridge, a C₂₋₃alkenylene bridge, a C₁₋₃ether bridge, or a C₁₋₃thioether bridge; or Y and a vicinal R⁶, together with the atoms to which they are attached, form fused-C₃₋₇cycloalkyl, fused-C₄₋₇cycloalkenyl, fused-heterocycloalkyl having 3-7 total ring atoms and 1-3 heteroatoms selected from N, O, and S, or fused-heterocycloalkenyl having 4-7 total ring atoms and 1 or 2 heteroatoms selected from N, O, and S; wherein the cycloalkyl, cycloalkenyl, heterocycloalkyl or heterocycloalkenyl of any of the foregoing is unsubstituted or substituted with 1-4 substituents, and each substituent independently is halo, C₁₋₃alkyl, C₁₋₃haloalkyl, C₀₋₂alkyleneOH, C₀₋₂alkyleneC₁₋₃alkoxy, or C₀₋₂alkyleneCN; and each R^{N1} independently is H or C₁₋₄alkyl. In some cases, X is

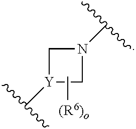

In some cases, X is

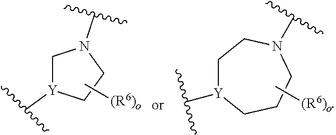

In some cases, X is

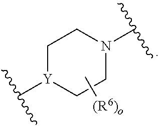

In some cases, X is

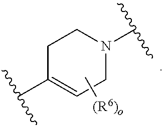

In some cases, Y is N. In some cases, Y is CH. In some cases, Y is C—F, C—Cl, C₂CH₃, C—CH₂CH₃, C—CH₂F, C—CHF₂, C—CF₃, C—OH, C—CH₂OH, C—OCH₃, or C—CH₂OCH₃. In some cases, o is 0. In some cases, o is 1. In some cases, o is 2. In some cases, each R⁶ independently is Br, Cl, F, CN, CH₃, CH₂F, CHF₂, CF₃, OH, CH₂OH, OCH₃, OCD₃, CH₂OCH₃, or CH₂N(CH₃)₂, or two geminal R⁶, together with the atom to which they are attached, form oxo, ═CH₂, spiro-cyclopropyl, spiro-cyclobutyl, spiro-oxetanyl, or spiro-tetrahydrofuranyl, or two vicinal R′, together with the atoms to which they are attached, form fused-cyclopropyl, fused-cyclobutyl, or fused-cyclopentyl, and any of the foregoing spiro and fused rings independently is unsubstituted or substituted with 1 or 2 substituents, and each substituent independently is halo, C₁₋₃alkyl, C₁₋₃haloalkyl, C₀₋₂alkyleneOH, C₀₋₂alkyleneC₁₋₃alkoxy, or C₀₋₂alkyleneCN. In some cases, each substituent independently is F, Cl, OH, OCH₃, OCH₂CH₃, or CN. In some cases, two non-neighboring R⁶ join together to form a C₀₋₃alkylene bridge, a C₂₋₆alkenylene bridge, a C₁₋₃ether bridge, or a C₁₋₃thioether bridge. In some cases, two non-neighboring R⁶ join together to form —CH₂—, —CH₂CH—, —CH₂CH₂CH₂—, —CH₂—CH═CH—, or —CH₂OCH₂—. In some cases. X is

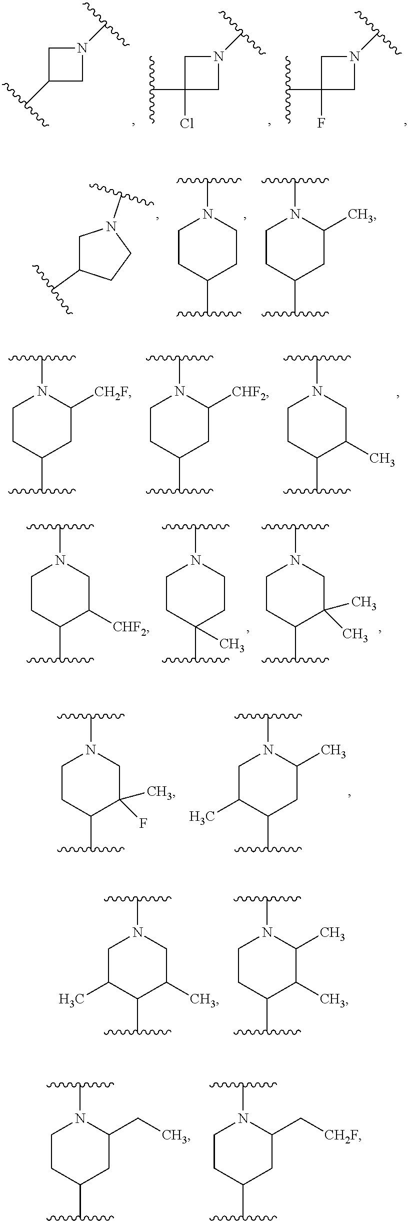

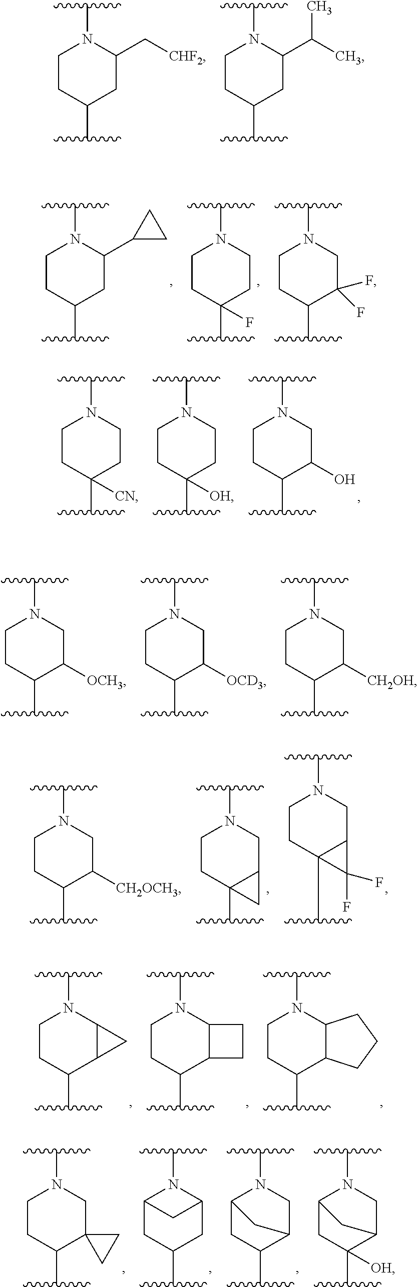

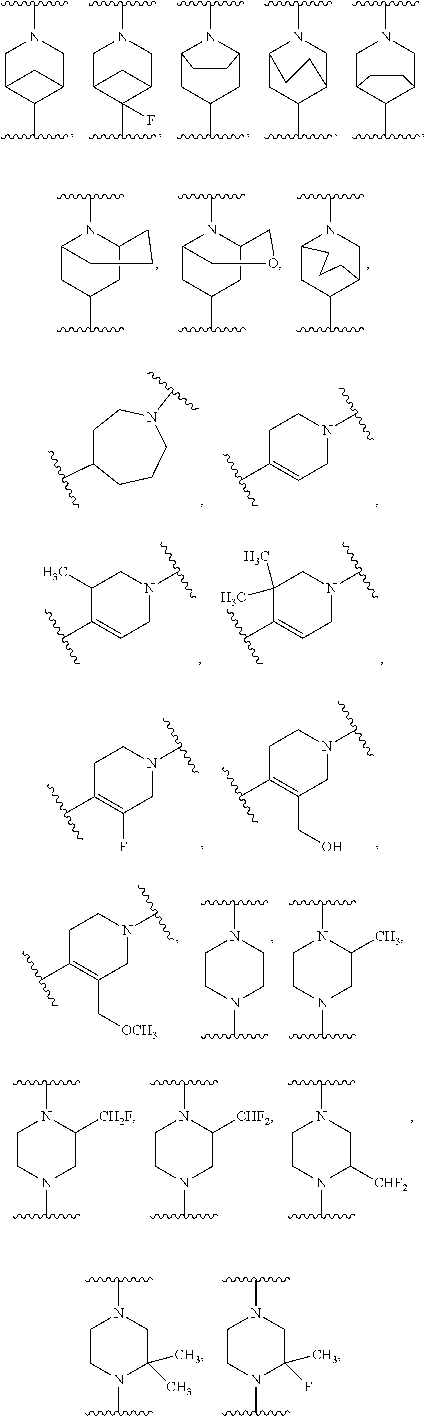

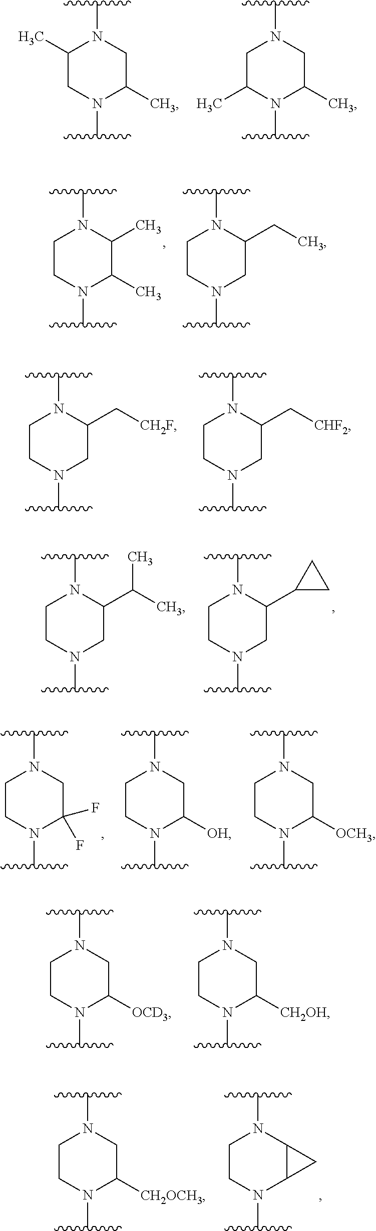

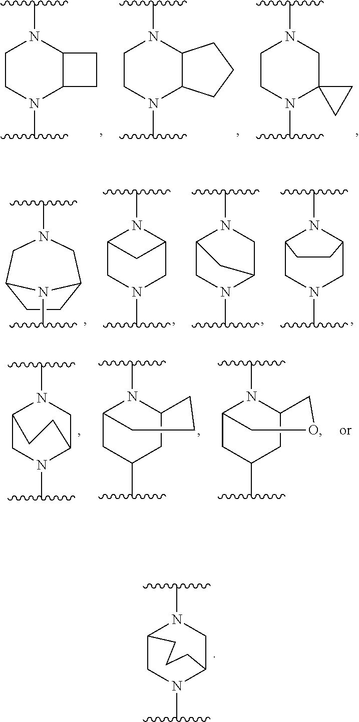

In some cases, X is

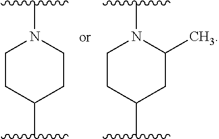

In some cases, Z is unsubstituted phenyl or phenyl substituted with 1-4 substituents, and each substituent independently is halo, C₀₋₃alkyleneCN, C₀₋₂alkyleneOH, C₀₋₃alkylene-C₁₋₄alkoxy, C₀₋₃alkylene-C₁₋₄thioalkoxy, or

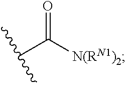

and each R^{A1} independently H or CH₃. In some cases, each substituent independently is F, Cl, CN, OCH₃, SCH₃, CH₂OH, or

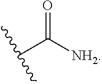 [SMILES: *C(N)=O]

In some cases, Z is

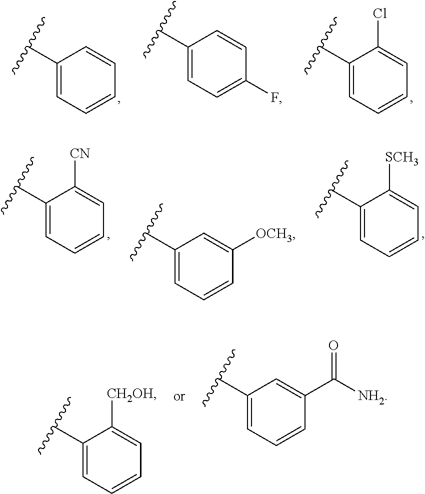

In some cases, Z is heteroaryl comprising 5 or 6 total ring atoms and 1-3 heteroatoms selected from N, O, and S, wherein the heteroaryl is unsubstituted or substituted with 1-4 substituents, and each substituent independently is halo, CN, C₁₋₆alkyl, C₁₋₆haloalkyl, C₂₋₆alkenyl, C₂₋₆ haloalkenyl. C₀₋₃alkylene-OH, C₀₋₆alkylene-C₁₋₃alkoxy, C₀₋₃alkylene-N(R^{N1})₂, C₀₋₂alkylene-C₃₋₆cycloalkyl, C₀₋₂alkylene-heterocycloalkyl having 3-6 total ring atoms and 1-3 heteroatoms selected from N, O, and S, or C₁₋₃alkylene-phenyl; wherein each of the C₁₋₆alkyl, C₃₋₆alkenyl, C₀₋₆alkylene-C₁₋₃alkoxy, cycloalkyl, heterocycloalkyl, and phenyl substituents independently is unsubstituted or substituted with 1-3 further substituents, and each further substituent independently is D, halo, C₁₋₃alkyl, C₁₋₃haloalkyl, C₁₋₂alkyleneOH, C₁₋₂alkylene-C₁₋₃alkoxy, C₁₋₃deuterated alkoxy, N(R^{N1})₂, (C═O)C₁₋₃alkyl, C₃₋₅cycloalkyl, heterocycloalkyl having 3-5 total ring atoms and 1 or 2 heteroatoms selected from N, O, and S; or two geminal further substituents, together with the atom to which they are attached, form spiro-C₃₋₅cycloalkyl, or spiro-heterocycloalkyl having 3-5 total ring atoms and 1 or 2 heteroatoms selected from N, O, and S; or two vicinal further substituents, together with the atoms to which they are attached, form fused-C₃₋₅cycloalkyl or fused-heterocycloalkyl having 3-5 total ring atoms and 1 or 2 heteroatoms selected from N, O, and S; wherein each of the foregoing cycloalkyl and heterocycloalkyl further substituents independently is unsubstituted or substituted with 1 or 2 substituents, and each substituent independently is halo or C₁₋₃alkyl; and each R^{N1} independently is H or C₁₋₃alkyl. In some cases, the heteroaryl is pyrrolyl, furanyl, thiophenyl, pyrazolyl, imidazolyl, thiazolyl, isothiazolyl, oxazolyl, isoxazolyl, triazolyl, thiadiazolyl, oxadiazolyl, pyridyl, pyridazinyl, pyrimidinyl, pyrazinyl, or triazinyl. In some cases, the heteroaryl is pyrazolyl or pyridyl. In some cases, the heteroaryl is substituted with 1 or 2 substituents. In some cases, each substituent independently is Br, Cl, F, CN, CF₃, CHF₂, CH₂F, CH₂CHF₂, CH₂CH₂F, CH(CH₂F) 2, CH(CH₃)CH₂F, CH(CH₃)CHF₂, C(═CH₂)CH₂F, OH, CH₂OH, CH₂CH₂OH, CH(CH₃)CH₂OH, C(CH₃)₂OH, C(CH₃)₂CH₂OH, CH₂C(CH₃)₂OH, NH₂, CH₂NH₂, CH₂NHCH₃, CH₂N(CH₃)₂, CH₂CH₂NH₂ CH₂CH₂NHCH₃, CH₂CH₂N(CH₃)₂, C₁₋₃alkyl selected from CH₃, CH₂CH₃, CH₂CH₂CH₃, and CH(CH₃)₂, C₂₋₆alkenyl selected from CH═CH₃, CH₂CH═CH₂, and CH═CHCH₃, C₀₋₃alkylene-C₁₋₃alkoxy selected from OCH₃, CH₂OCH₃, CH—CH₂OCH₃, CH₂CH₂OCH₂CH₃, CH₂CH₂CH₂OCH₃, CH(CH₃)OCH₃, CH(CH)CH₂OCH₃, CH(OCH₃)CH₂OCH₃, CH(CH₃)(OCH₃)CH₂OCH₃, C(CH₃)₂OCH₃, C(CH₃)₂CH₂OCH₃, CH₂CH(CH₃)OCH₃, CH, (CH₃)(OCH₃)OCH₃, CH₂C(CH₃)₂OCH₃, and CHIC(CH₃)₂OCH₃, cycloalkyl selected from cyclopropyl, cyclobutyl, cyclopentyl, and cyclohexyl, or heterocycloalkyl selected from azetidinyl, pyrrolidinyl, piperidinyl, pyrazolidinyl, oxetanyl, tetrahydrofuranyl, tetrahydropyranyl, isoxazolidinyl, and morpholinyl; wherein each of the C₁₋₆alkyl, C₂₋₆alkenyl, C₀₋₆alkylene-C₁₋₃alkoxy, cycloalkyl, and heterocycloalkyl substituents independently is unsubstituted or substituted with 1-3 further substituents and each further substituent independently is D, halo, C₁₋₃alkyl, C₁₋₃haloalkyl, C₁₋₂alkyleneOH, C₁₋₂alkylene-C₁₋₃alkoxy, C₁₋₃deuterated alkoxy, N(R^{N1})₂, (C═O)C₁₋₃alkyl, C₃₋₅cycloalkyl, heterocycloalkyl having 3-5 total ring atoms and 1 or 2 heteroatoms selected from N, O, and S, or two geminal further substituents, together with the atom to which they are attached, form C₃₋₅spiro-cycloalkyl, or spiro-heterocycloalkyl having 3-5 total ring atoms and 1 or 2 heteroatoms selected from N, O, and S; or two vicinal further substituents, together with the atoms to which they are attached, form fused-C₃₋₅cycloalkyl or fused-heterocycloalkyl having 3-5 total ring atoms and 1 or 2 heteroatoms selected from N, O, and S. In some cases, each further substituent independently is D, Br, Cl, F, OH, CH₃, CF₃, CF₂H, CFH₂, OCH₃, OCD₃, CH₂OCH₃, N(CH₃)₂, (C═O)CH₃, oxetanyl, or azetidinyl, or two geminal further substituents, together with the atom to which they are attached, form spiro-oxetanyl or spiro-azetidinyl; wherein each of the foregoing oxetanyl, azetidinyl, spiro-oxetanyl, and spiro-azetidinyl independently is unsubstituted or substituted with F, CH₃, or a combination thereof. In some cases, each further substituent independently is D. Br, Cl, F, OH, CH₃, CF₃. CF₂H, CFH₂, OCH₃, OCD₃, N(CH₃), (C═O)CH₃,

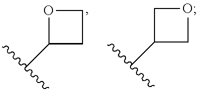 [SMILES: *C1CCO1 ; *C1COC1]

or two geminal further substituents, together with the atom to which they are attached, form

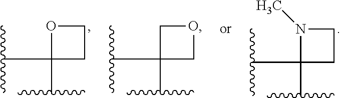

In some cases, each substituent of the heteroaryl of Z independently is Cl, F, CN, CH₃, CD₃, CH₂CH₃, CH(CH₃)₂, CF₃, CHF₂, CH₂F, CH₂CHF₂, CH₂CH₂F, CH(CH₂F) 2, CH(CH)CH₂F, CH(CH₃)CHF₂, C(═CH₂)CH₂F, OH, CH₂OH, CH₂CH₂OH, CH(CH₃)CH₂OH, C(CH₃)₂OH, C(CH₃)₂CH₂OH, CH₂C(CH₃)₂OH, OCH₃, OCD₃, CH₂OCH₃, CH₂OCD₃, CH₂CH₂OCH₃, CHFCH₂OCH₃, CF₂CH₂OCH₃, CH₂CH₂OCD₃, CH₂CH₂OCH₂CH₃, CH₂CH₂CH₂OCH₃, CH₂CH₂CH₂OCD₃, CH(CH₃)OCH₃, CH(CH₃)CH₂OCH₃, CH(OCH,)CH₂OCH₃, CH(CH₃)(OCH₃)CH₂OCH₃, CH(CH₂F) (CH)CH₂OCD₃, CH(CH₃)CH₂OCD₃, C(CH₃)₂OCH₃, C(CH₃)₂CH₂OCH₃, C(CH₃)₂CH₂OCD₃, CH₂CH(CH₃)OCH₃, CH, (CH₃)(OCH₃)OCH₃, CH₂CH(CH₃)OCD₃, CH₂C(CH₃)₂OCH₃, CH₂C(CH₃)₂OCD₃, NH₂, CH₂NH₂, CH₂NHCH₃, CH₂N(CH₃)₂, CH₂CH₂NH₂, CH₂CH₂NHCH₃, CH₂CH₂N(CH₃)₂,

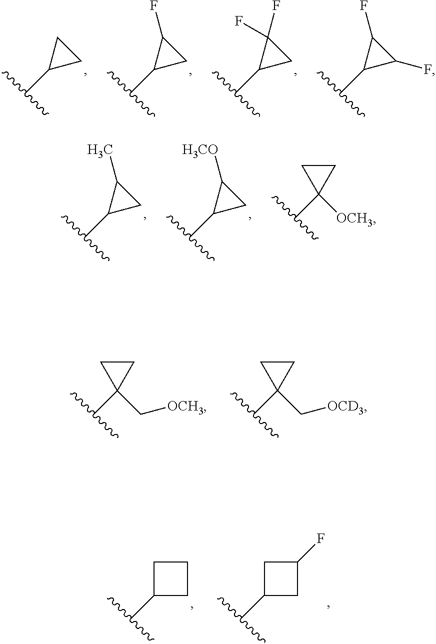

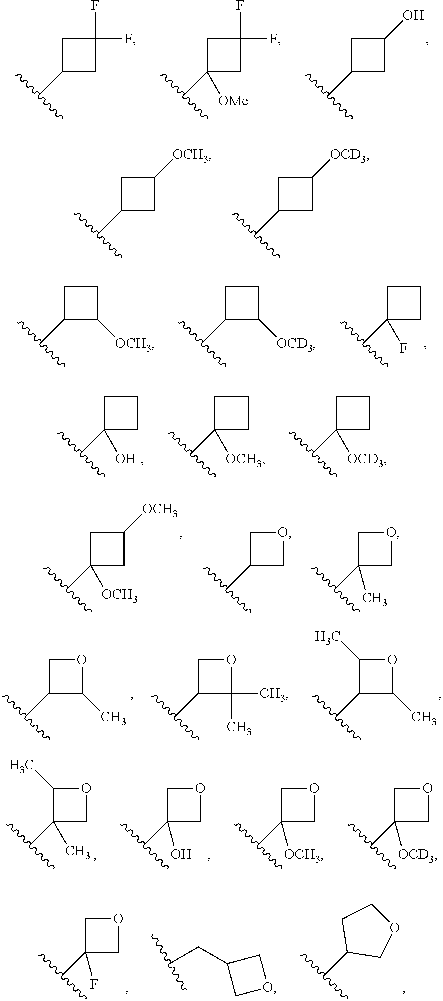

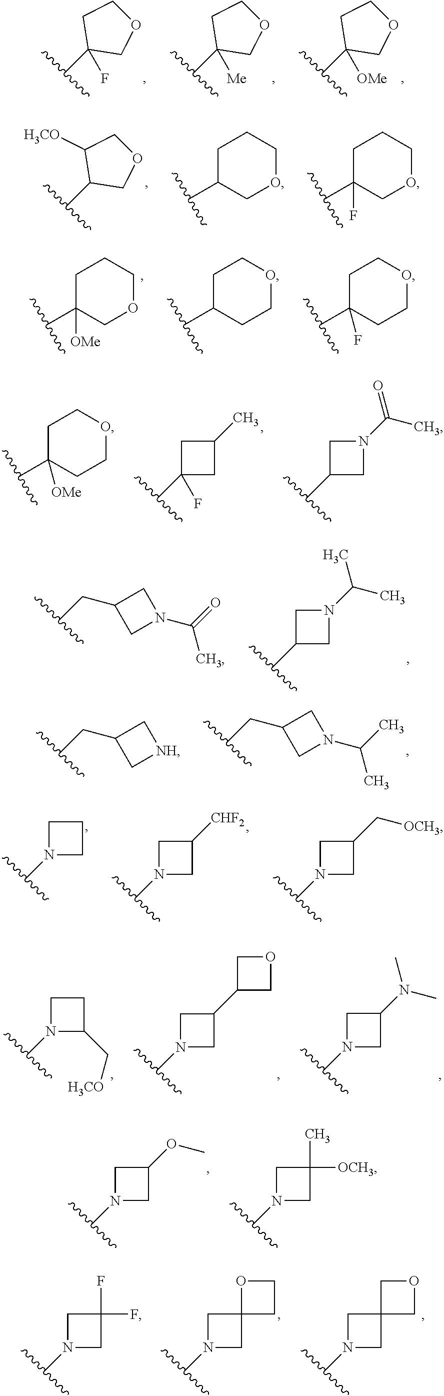

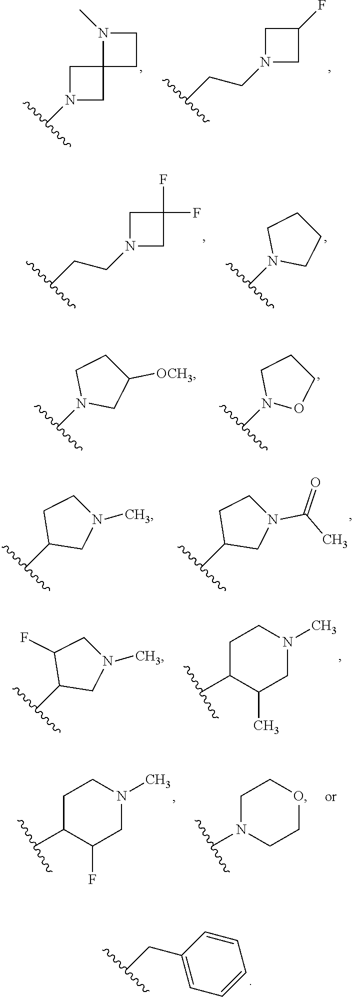

In some cases, each substituent of the heteroaryl of Z independently is CH₃, C(CH₃)₂CH₂OH, CH₂CH₂OCH₃, CH₂CH₂OCD₃, CH(CH₃)OCH₃,

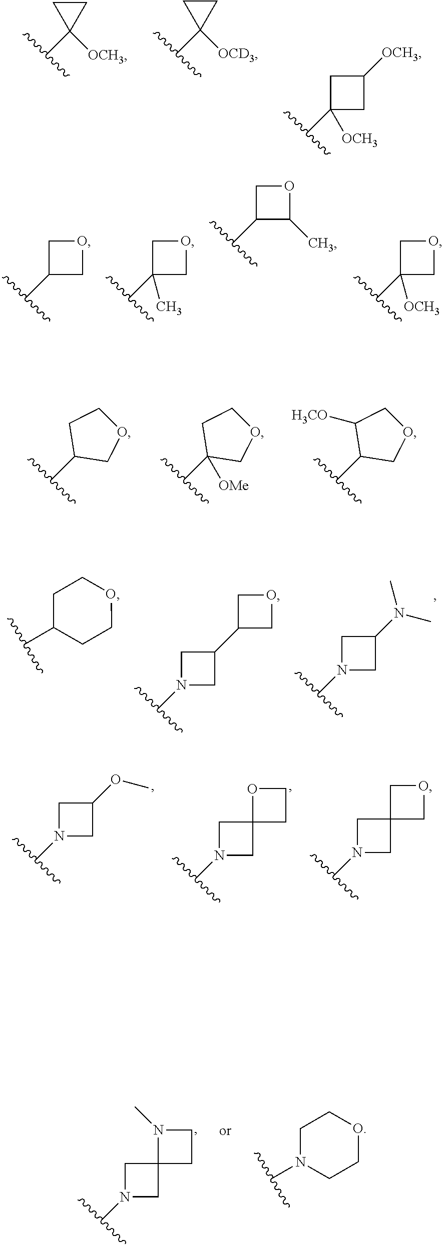

In some cases, each substituent of the heteroaryl of Z independently is CH₃, CH₂CH₂OCH₃,

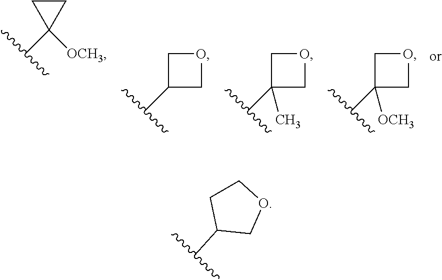

or any combination of the foregoing.

In some cases, Z is

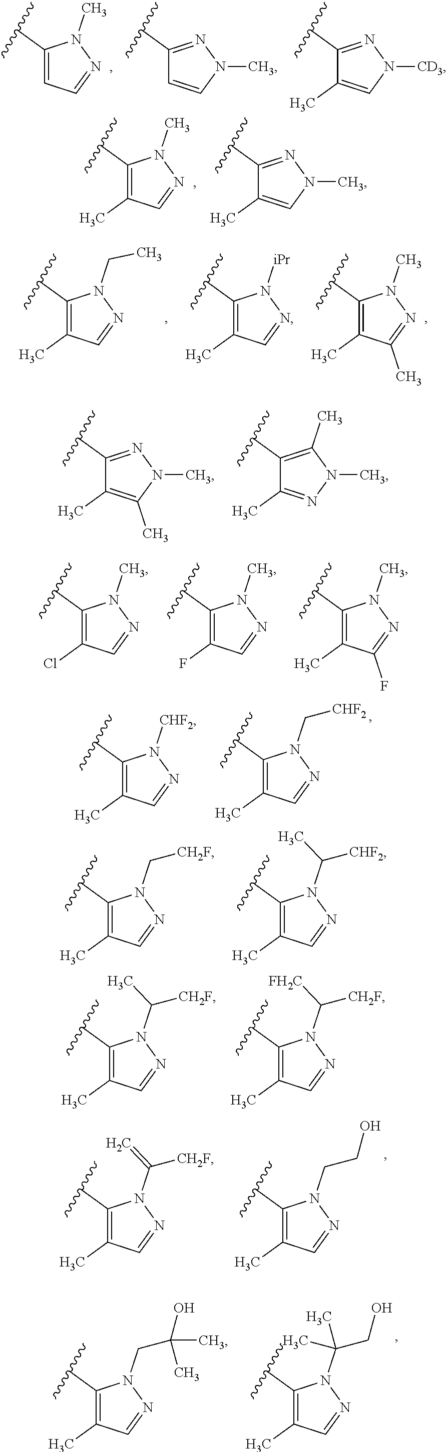

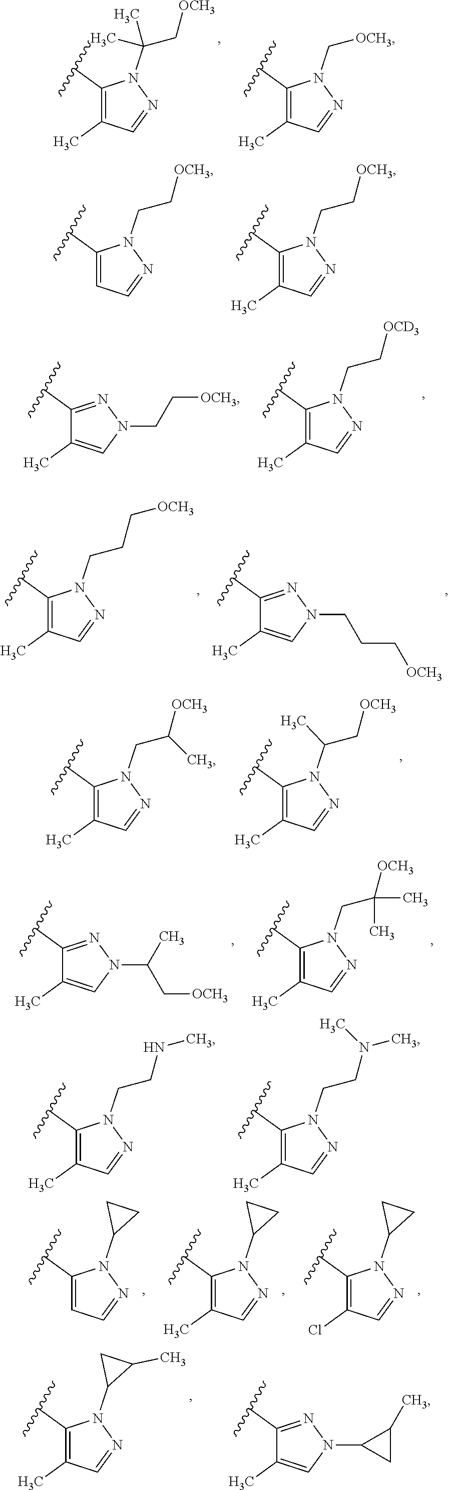

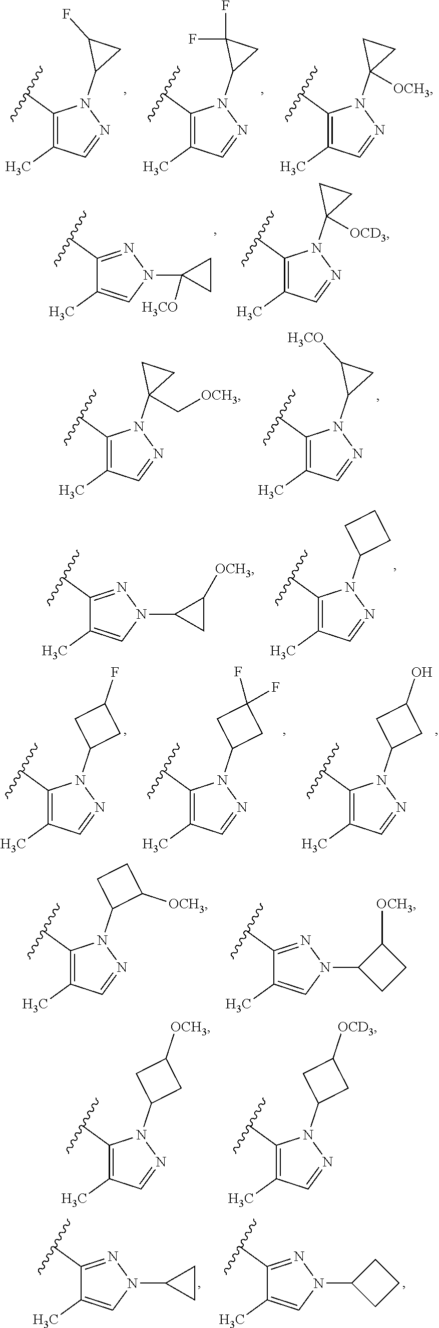

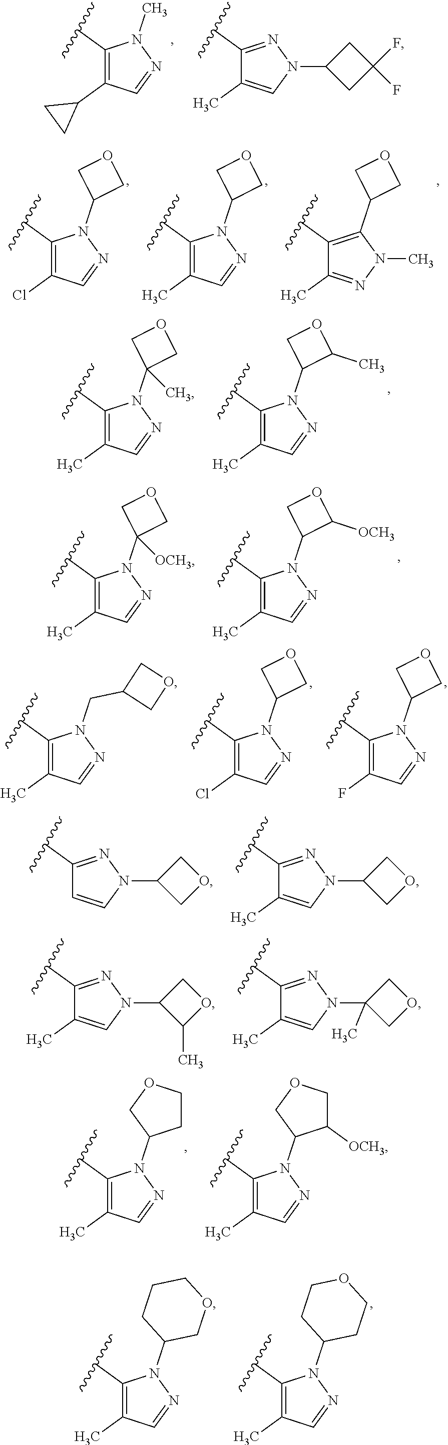

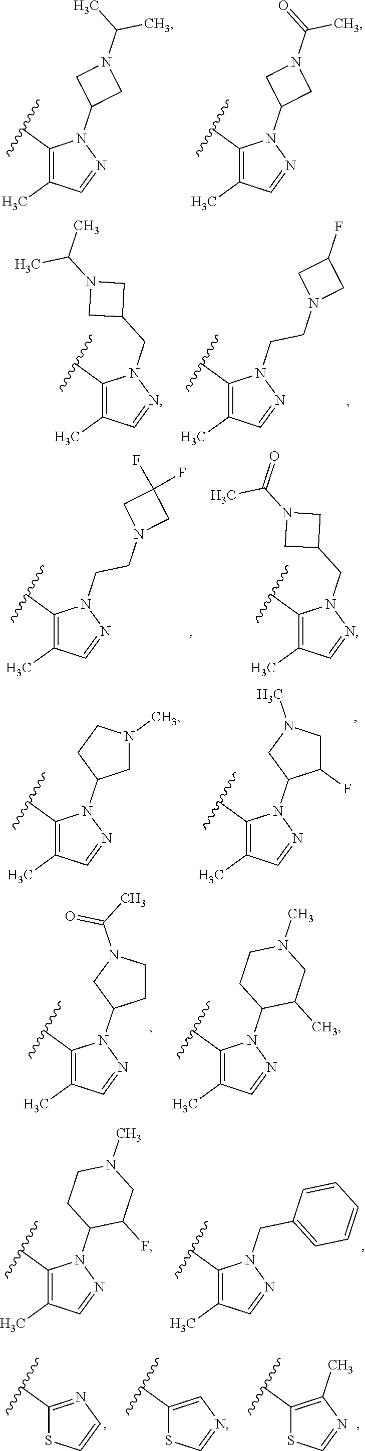

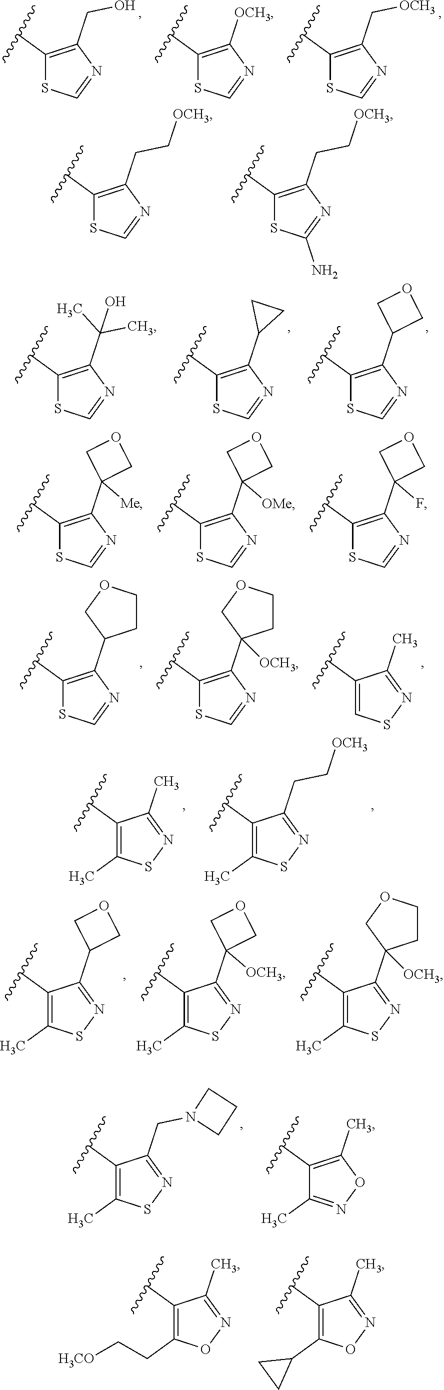

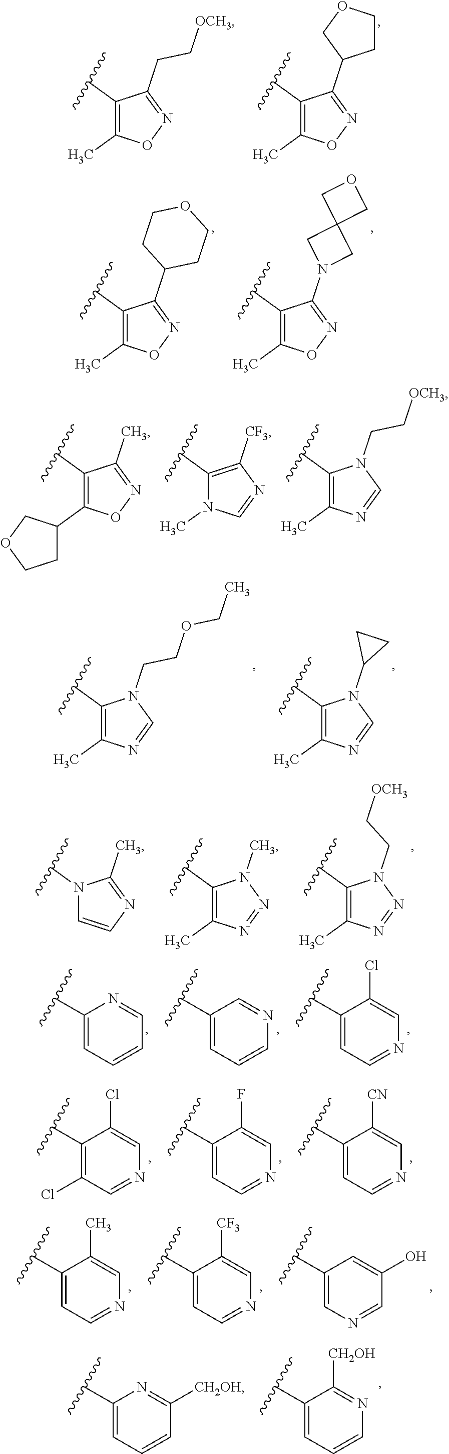

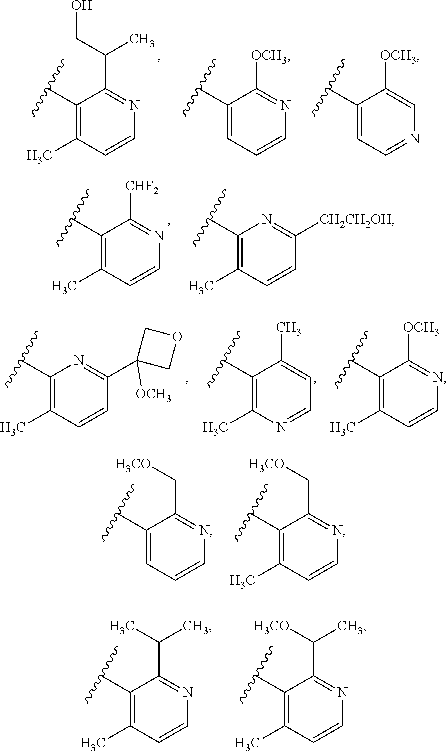

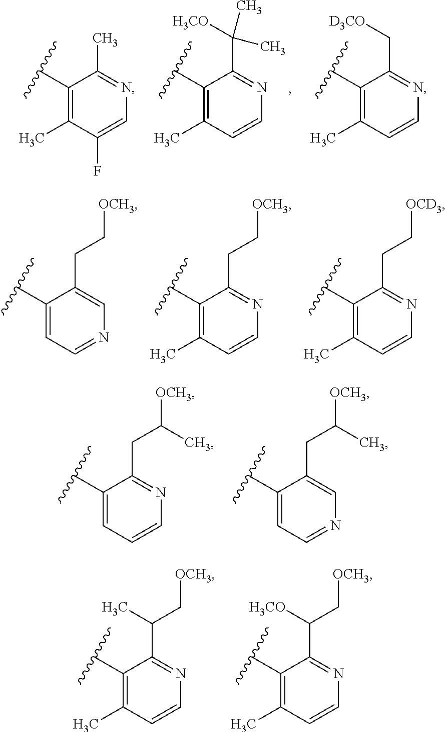

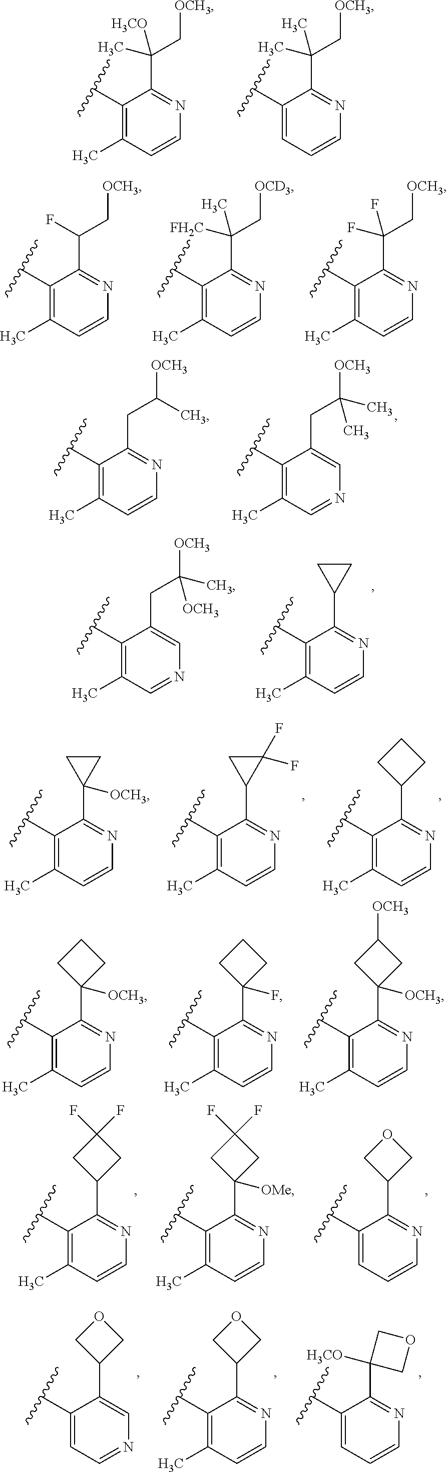

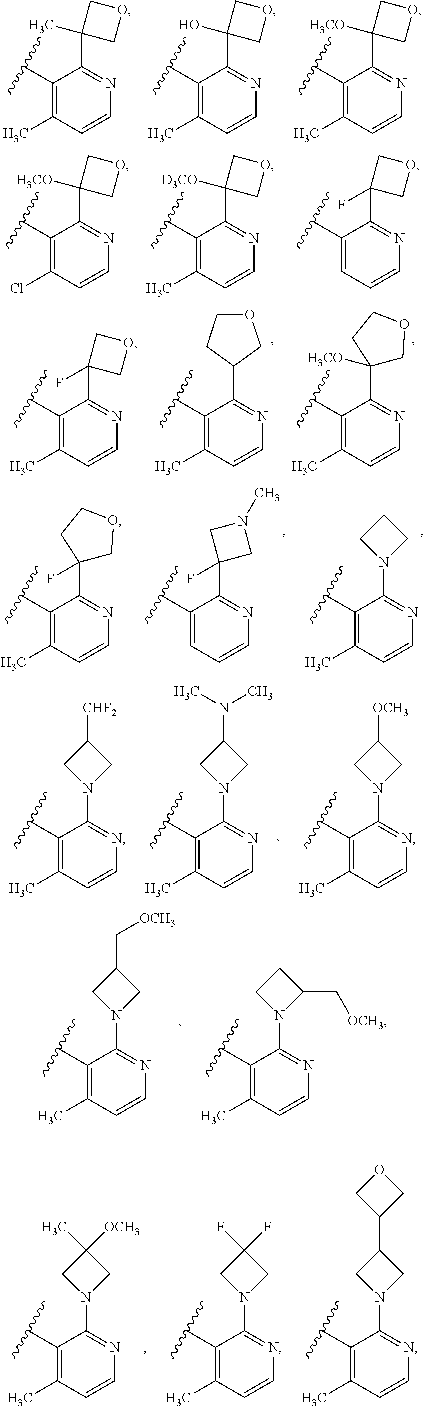

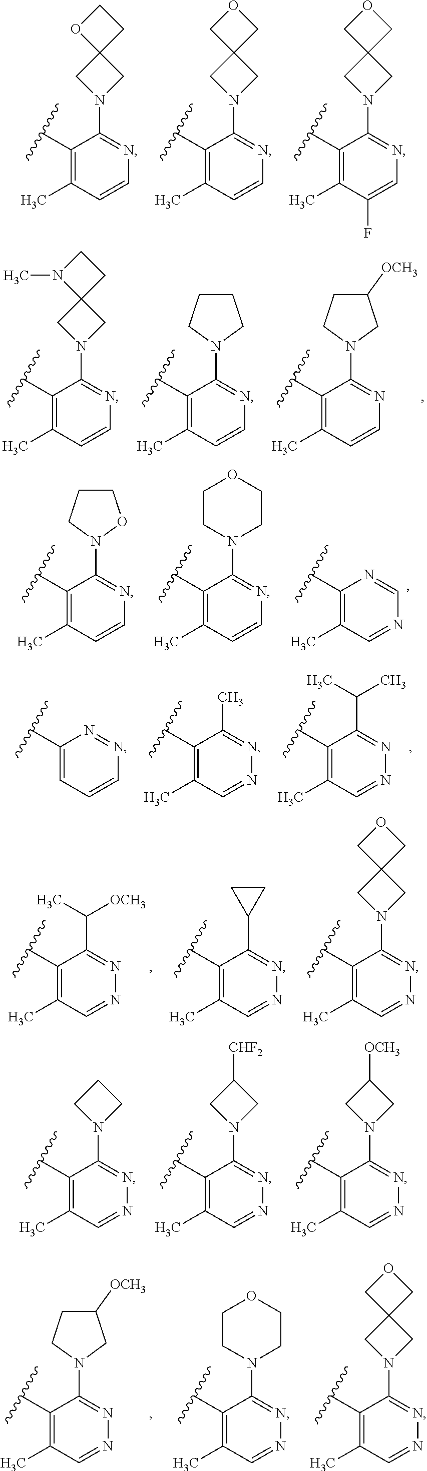

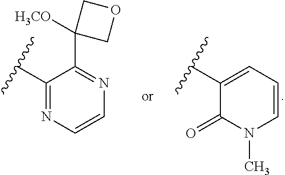

In some cases, Z is

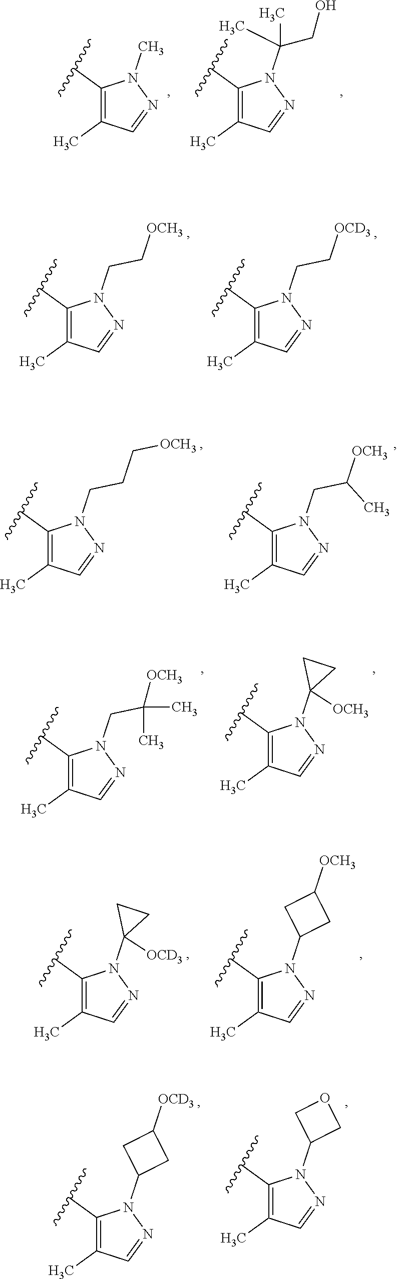

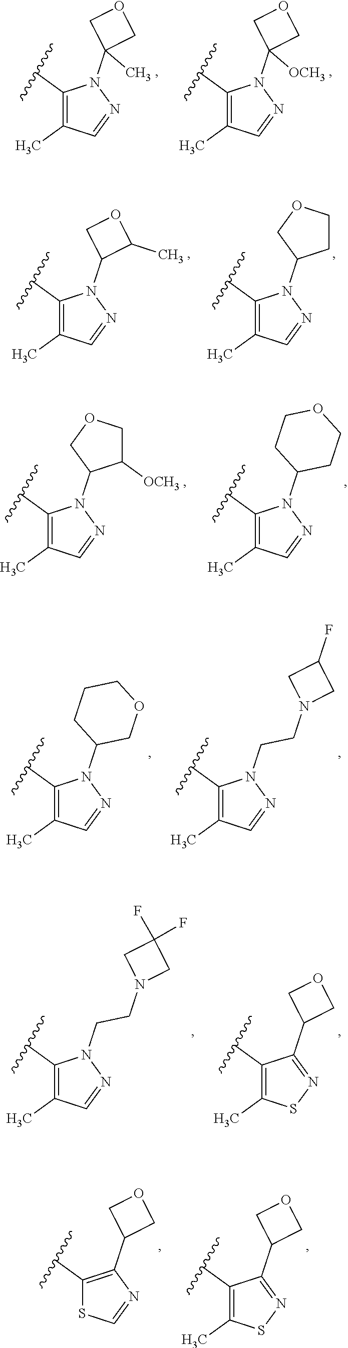

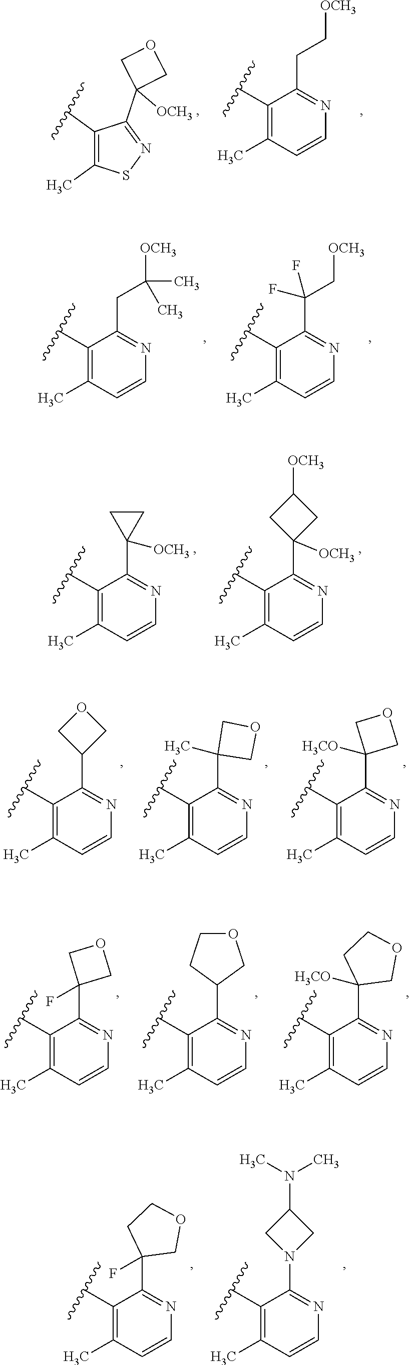

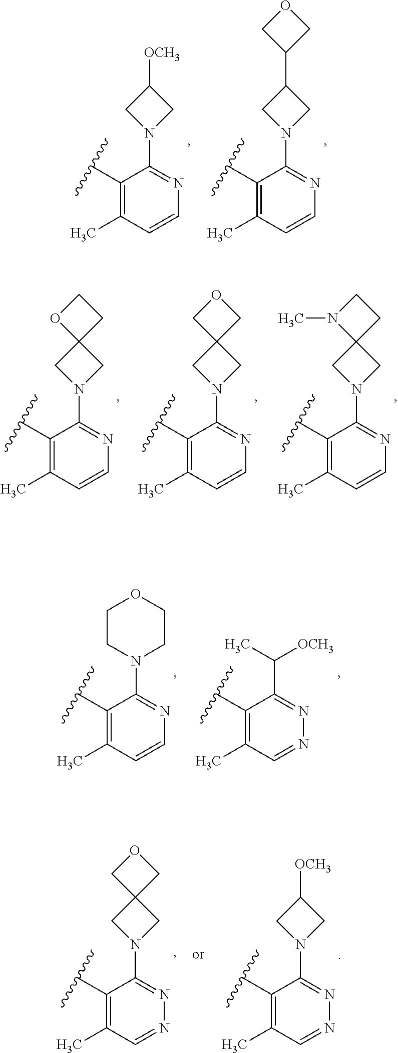

In some cases, Z is

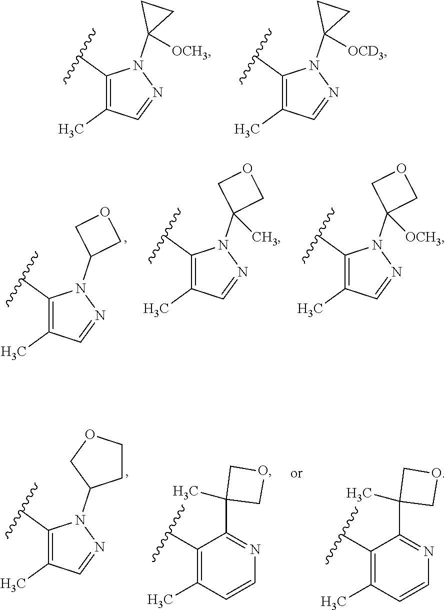

In some cases, Z is a bicyclic ring comprising heteroaryl having 5 or 6 total ring atoms and 1-3 heteroatoms selected from N, O, and S fused to C₅₋₆cycloalkyl or a heterocycloalkyl ring having 5 or 6 total ring atoms and 1 or 2 heteroatoms selected from N, O, and S; wherein the bicyclic ring is unsubstituted or substituted with 1-4 substituents, and each substituent independently is halo, CN, C₁₋₆alkyl, C₁₋₆haloalkyl, C₀₋₃alkylene-OH, or C₀₋₆alkylene-C₁₋₃alkoxy. In some cases, Z is

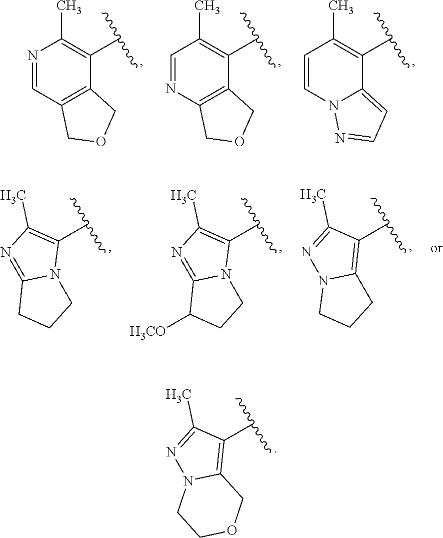

In some cases,

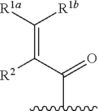

is

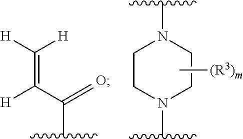

is

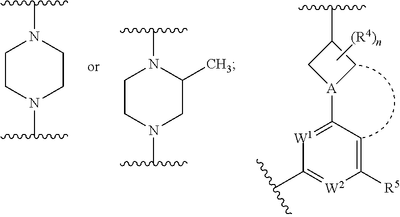

is

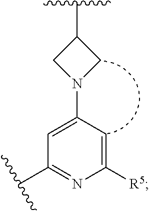

R⁵ is CHF₂ or CF₃; X is

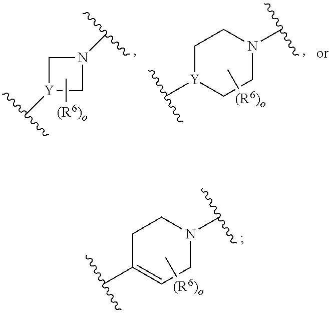

o is 0 or 1; R⁶ is CH₃; Y is CH or N; and Z is pyrazolyl or pyridyl, each of which is substituted with 1 or 2 substituents, and each substituent independently is Cl, F, CN, CH₃, CD₃, CH₂CH₃, CH(CH₃)₂, CF₃, CHF₂, CH₂F, CH₂CHF₂, CH₂CH₂F, CH(CH₂F) 2, CH(CH)CH₂F, CH(CH₃)CHF₂, C(═CH,)CH₂F, OH, CH₂OH, CH₂CH₂OH, CH(CH₃)CH₂OH, C(CH₃)₂OH, C(CH₃)₂CH₂OH, CH₂C(CH₃)₂OH, OCH₃, OCD₃, CH₂OCH₃, CH₂OCD₃, CH₂CH₂OCH₃, CHFCH₂OCH₃, CF₂CH₂OCH₃, CH₂CH₂OCD₃, CH₂CH₂OCH₂CH₃, CH₂CH₂CH₂OCH₃, CH₂CH₂CH₂OCD₃, CH(CH₃)OCH₃, CH(CH₃)CH₂OCH₃, CH(OCH₃)CH₂OCH₃, CH(CH₃)(OCH₃)CH₂OCH₃, CH(CH₂F) (CH)CH₂OCD₃, CH(CH₃)CH₂OCD₃, C(CH₃)₂OCH₃, C(CH₃)₂CH₂OCH₃, C(CH₃)₂CH₂OCD₃, CH₂CH(CH₃)OCH₃, CH₂(CH₃)(OCH₃)OCH₃, CH₂CH(CH₃)OCD₃, CH₂C(CH₃)₂OCH₃, CH₂C(CH₃)₂OCD₃, NH₂, CH₂NH₂, CH₂NHCH₃, CH₂N(CH₃)₂, CH₂CH₂NH₂, CH₂CH₂NHCH₃, CH₂CH₂N(CH₃)₂,

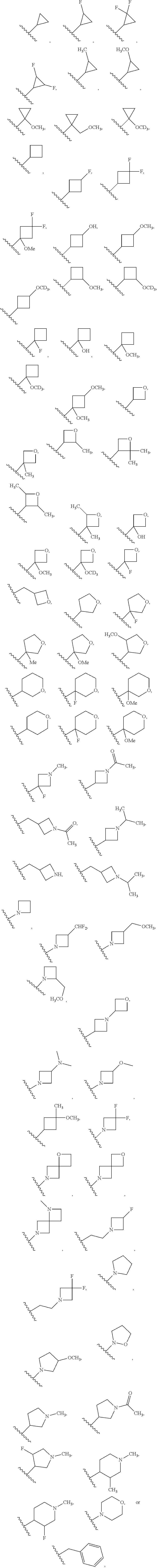

In some cases, X is

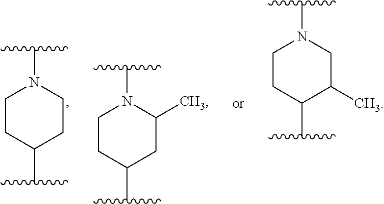

In some cases, [symbol] is

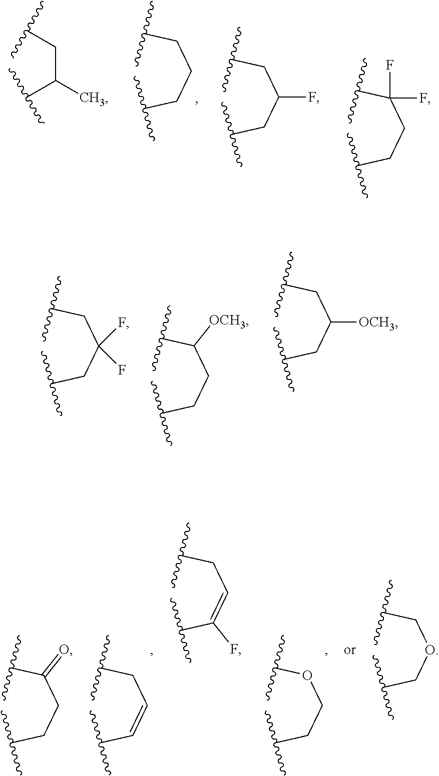

In some cases, [symbol] is

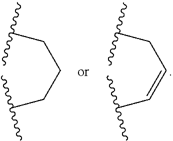 [SMILES: *CCC* ; */C=C\C*]

In some cases, Z is

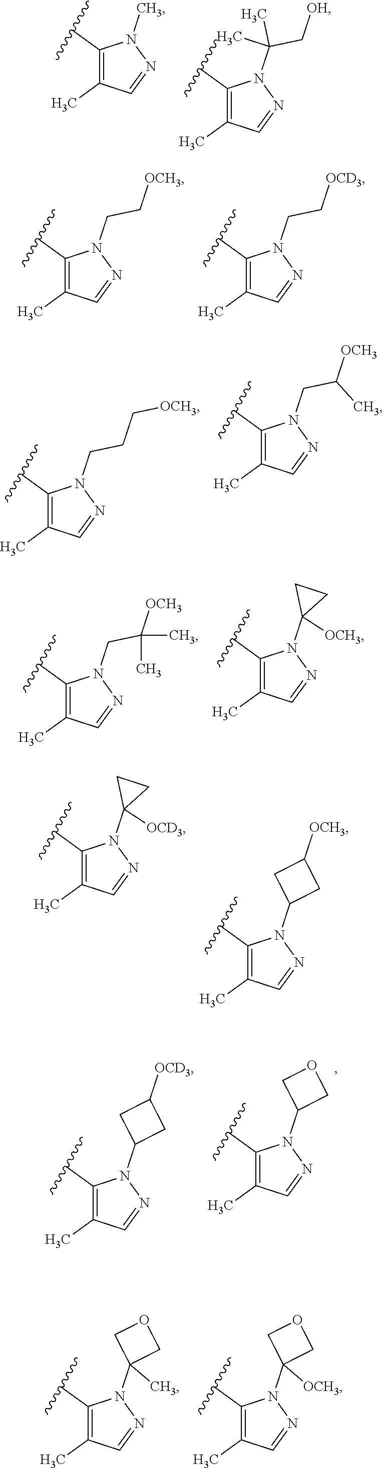

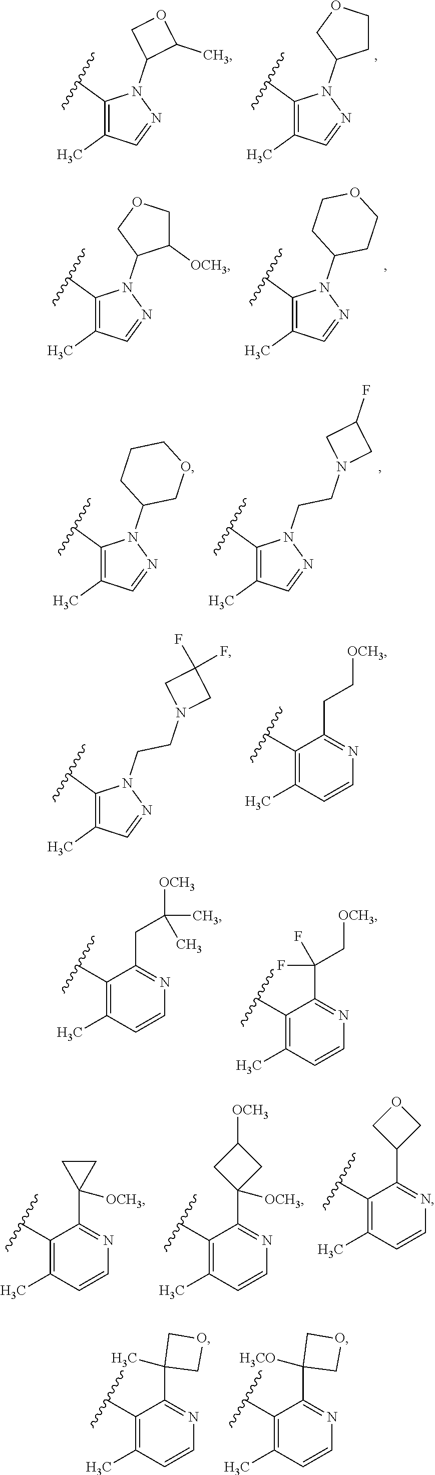

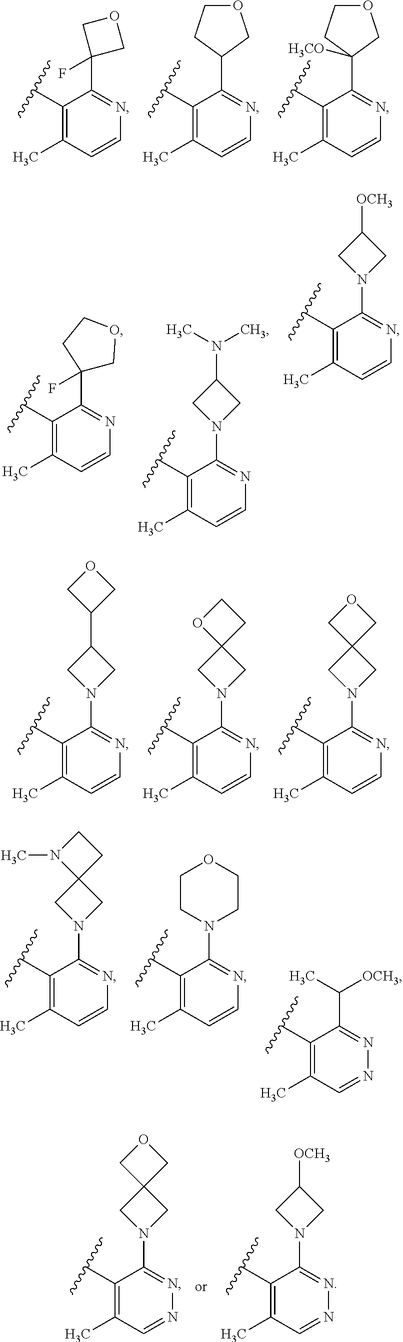

In some cases, Z is

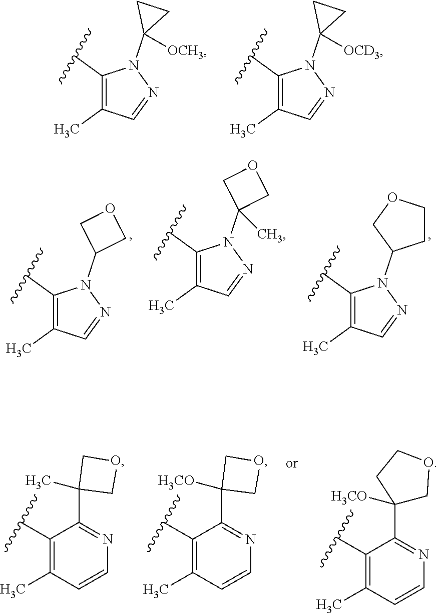

In some cases, the compound of Formula (I) is a compound listed in Table A, or a pharmaceutically acceptable salt thereof. In some cases, the compound of Formula (I) is a compound listed in Table B, or a pharmaceutically acceptable salt thereof. In some cases, the compound of Formula (I) is a compound listed in Table A′, or a pharmaceutically acceptable salt thereof. In some cases, the compound of Formula (I) is a compound listed in Table B′ or a pharmaceutically acceptable salt thereof.

Another aspect of the disclosure provides a pharmaceutical composition comprising a compound or salt described herein, such as a compound of Formula (I), Formula (I′), Formula (IA), Formula (IB), Formula (IE), Formula (IF), Formula (IG), Formula (II), Formula (II′), Formula (IIA), Formula (IIB), Formula (IIC), Formula (IID), Formula (IIE), and Formula (IIF), or a compound listed in Table A, Table A′, Table B, Table B′, and Table E, or a pharmaceutically acceptable salt of any of the foregoing, and a pharmaceutically acceptable excipient.

Yet another aspect of the disclosure provides a method of treating cancer in a subject in need of treatment, the method comprising administering to the subject a therapeutically effective amount of the compound or salt described herein, such as a compound of Formula (I), Formula (I′), Formula (IA), Formula (IB), Formula (IE), Formula (IF), Formula (IG), Formula (II), Formula (II′), Formula (IIA), Formula (IIB), Formula (IIC), Formula (IID). Formula (IIE), and Formula (IIF), or a compound listed in Table A, Table A, Table B, Table B′, and Table E, or a pharmaceutically acceptable salt of any of the foregoing, or a pharmaceutical composition described herein. In some cases, the subject has one or more cancer cells express that express KRAS G12C mutant protein. In some cases, the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, a solid tumor, or any combination of the foregoing. In some cases, the cancer is non-small cell lung cancer, colorectal cancer, pancreatic cancer, appendiceal cancer, endometrial cancer, esophageal cancer, cancer of unknown primary, ampullary cancer, gastric cancer, small bowel cancer, sinonasal cancer, bile duct cancer, melanoma, a solid tumor, or any combination of the foregoing. In some cases, the cancer is non-small cell lung cancer. In some cases, the cancer is colorectal cancer. In some cases, the cancer is pancreatic cancer. In some cases, the cancer is solid tumor. In some cases, the subject has a cancer that was determined to have one or more cells expressing the KRAS G12C mutant protein prior to administration of the compound, salt, or pharmaceutical composition. In some cases, the method further comprises simultaneous, separate, or sequential administration of an effective amount of a second compound, wherein the second compound is an ATR inhibitor, Aurora kinase A inhibitor, AKT inhibitor, arginase inhibitor, CDK2 inhibitor, CDK4/6 inhibitor, ErbB family inhibitor, ERK inhibitor, FAK inhibitor, FGFR inhibitor, glutaminase inhibitor, IGF-IR inhibitor, KIF18A inhibitor, MAT2A inhibitor, MCL-1 inhibitor, MEK inhibitor, mTOR inhibitor, PARP inhibitor, PD-1 inhibitor, PD-L1 inhibitor, PI3K inhibitor, PRMT5 inhibitor. Raf kinase inhibitor, SHP2 inhibitor, SOS1 inhibitor, Src kinase inhibitor, or one or more chemotherapeutic agents.

Another aspect of the disclosure provides a compound described herein, such as a compound of Formula (I), Formula (I), Formula (IA), Formula (IB), Formula (IE), Formula (IF), Formula (IG), Formula (II), Formula (II′), Formula (IIA), Formula (IIB), Formula (IIC). Formula (IID), Formula (IIE), and Formula (IIF), or a compound listed in Table A, Table A′, Table B, Table B′, and Table E, or a pharmaceutically acceptable salt of any of the foregoing, or a pharmaceutical composition described herein, for use as a medicament. The disclosure also provides the use of a compound described herein, such as a compound of Formula (D), Formula (I′), Formula (IA), Formula (IB), Formula (IE), Formula (IF), Formula (IG), Formula (II), Formula (II′), Formula (IIA), Formula (IIB), Formula (IIC), Formula (IID), Formula (IIE), and Formula (IIF), or a compound listed in Table A, Table A′, Table B, Table B′, and Table E, or a pharmaceutically acceptable salt of any of the foregoing, or a pharmaceutical composition described herein for the manufacture of a medicament for the treatment of cancer. In some cases, the disclosure provides a compound, such as a compound of Formula (I), Formula (I′), Formula (IA), Formula (IB), Formula (IE), Formula (IF), Formula (IG), Formula (II), Formula (II′), Formula (IIA), Formula (IIB), Formula (IIC), Formula (IID), Formula (IIE), and Formula (IIF), or a compound listed in Table A, Table A′, Table B, Table B′, and Table E, or a pharmaceutically acceptable salt of any of the foregoing, or a composition described herein for use in treating cancer. In some cases, one or more cancer cells express KRAS G12C mutant protein in any of the uses described herein. In some cases, the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, a solid tumor, or any combination of the foregoing. In some cases, the cancer was determined to have one or more cells expressing the KRAS G12C mutant protein prior to administration of the compound, salt, or pharmaceutical composition.

Another aspect of the disclosure provides an intermediate selected from:
(a) a compound of Formula (Int-AA):

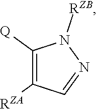

Formula (Int-AB):

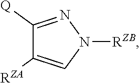

Formula (Int-AC):

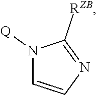

Formula (Int-AD):

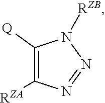

Formula (Int-AE):

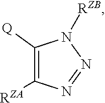

Formula (Int-AF):

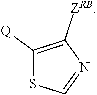

Formula (Int-AG):

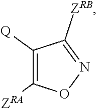

Formula (Int-AH):

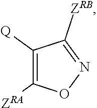

Formula (Int-AI):

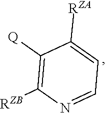

or Formula (Int-AJ):

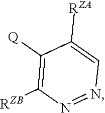

or a pharmaceutically acceptable salt of any of the foregoing; or
(b) a compound of Formula (Int-B):

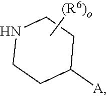

a nitrogen-protected analog thereof, or a pharmaceutically acceptable salt of any of the foregoing: or
(c) a compound of Formula (Int-C):

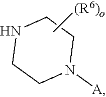

a nitrogen-protected analog thereof, or a pharmaceutically acceptable salt of any of the foregoing; or
(d) a compound of Formula (Int-D):

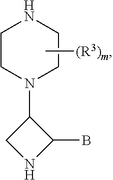

a nitrogen-protected analog thereof, or a pharmaceutically acceptable salt of any of the foregoing; or
(e) a compound of Formula (Int-E):

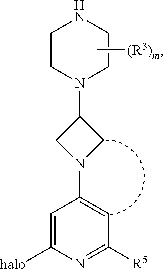

a nitrogen-protected analog thereof, or a pharmaceutically acceptable salt of any of the foregoing:
wherein:
A is

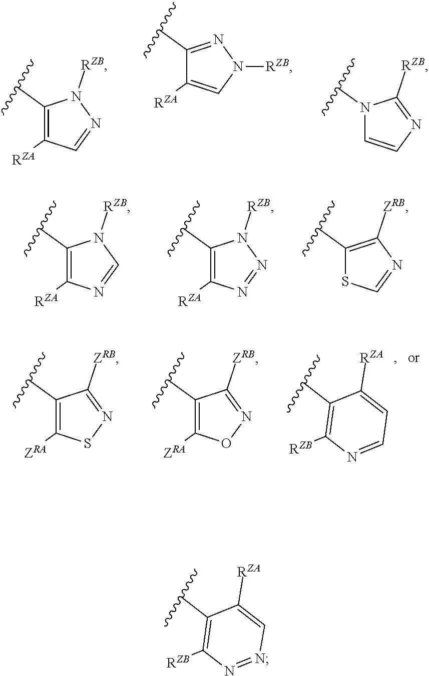

B is C₁₋₃alkylene-CH═CH, or C₁₋₃alkyleneOH;
Q is F, Cl, Br, I, or an organoborane;
m is 0, 1, 2, 3, or 4;
o is 0, 1, 2, 3, or 4;
halo is F, Cl, Br, or I;
[symbol] is C₂₋₆alkylene, C₃₋₆alkenylene, heteroalkylene having 2-6 total atoms and 1-3 heteroatoms selected from N, O, and S, or heteroalkenylene having 3-6 total atoms and 1 or 2 heteroatoms selected from N, O, and S, wherein [symbol] is unsubstituted or substituted with 1-4 substituents, and each substituent independently is C₁₋₃alkyl, C₁₋₃haloalkyl, C₂₋₃alkenyl, halo, CN, C₀₋₃alkyleneOH, C₀₋₃alkylene-C₁₋₃alkoxy, C₃₋₅cycloalkyl, C₄₋₅cycloalkenyl, heterocycloalkyl having 4 or 5 total ring atoms and 1-3 heteroatoms selected from N, O, and S, heterocycloalkenyl having 4 or 5 total ring atoms and 1-3 heteroatoms selected from N, O, and S, or phenyl; or two geminal substituents, together with the atom to which they are attached, form oxo, ═CH₂, spiro-C₃₋₅cycloalkyl, spiro-C₄₋₅cycloalkenyl, spiro-heterocycloalkyl having 3-5 total ring atoms and 1 or 2 heteroatoms selected from N, O and S, or spiro-heterocycloalkenyl having 4 or 5 total ring atoms and 1 or 2 heteroatoms selected from N, O and S; or two vicinal substituents, together with the atoms to which they are attached, form fused-C₃₋₅cycloalkyl, fused-C₄₋₅cycloalkenyl, fused-heterocycloalkyl having 3-5 total ring atoms and 1 or 2 heteroatoms selected from N, O and S or fused-heterocycloalkenyl having 4 or 5 total ring atoms and 1 or 2 heteroatoms selected from N, O and S;
each of R^{ZA} and R^{ZB} independently is halo, CN, C₁₋₆alkyl, C₁₋₆haloalkyl, C₂₋₆alkenyl, C₂₋₆haloalkenyl, C₀₋₆alkylene-OH, C₀₋₃alkylene-C₁₋₃alkoxy, C₀₋₂alkylene-N(R^{N1})₂, C₀₋₂alkylene-C₃₋₆cycloalkyl, C₀₋₂alkylene-heterocycloalkyl having 3-6 total ring atoms and 1-3 heteroatoms selected from N, O, and S, or C₀₋₂alkylene-phenyl;
   wherein each of the C₁₋₆alkyl, C₃₋₆alkenyl, C₀₋₆alkylene-C₁₋₃alkoxy, cycloalkyl, heterocycloalkyl, and phenyl substituents independently is unsubstituted or substituted with 1-3 further substituents, and each further substituent independently is D, halo, C₁₋₃alkyl, C₁₋₃haloalkyl, C₁₋₂alkyleneOH, C₁₋₂alkylene-C₁₋₃alkoxy, Ct-deuterated alkoxy, N(R^{N1})₂, (C═O) C₁₋₃alkyl, C₃₋₅cycloalkyl, heterocycloalkyl having 3-5 total ring atoms and 1 or 2 heteroatoms selected from N, O, and S; or two geminal further substituents, together with the atom to which they are attached, form spiro-C₃₋₅cycloalkyl, or spiro-heterocycloalkyl having 3-5 total ring atoms and 1 or 2 heteroatoms selected from N, O, and S; or two vicinal further substituents, together with the atoms to which they are attached, form fused-C₃₋₅cycloalkyl or fused-heterocycloalkyl having 3-5 total ring atoms and 1 or 2 heteroatoms selected from N, O, and S; wherein each of the foregoing cycloalkyl and heterocycloalkyl further substituents independently is unsubstituted or substituted with 1 or 2 substituents, and each substituent independently is halo or C₁₋₃alkyl; and each R^{N1} independently is H or C₁₋₃alkyl.
each R³ independently is C₁₋₃alkyl, C₁₋₃haloalkyl,

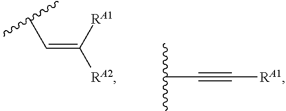

C₀₋₃alkyleneCN, C₀₋₃alkyleneOH, or C₀₋₃alkylene-C₁₋₃alkoxy; or two geminal R³, together with the atom to which they are attached, form oxo, spiro-C₃₋₇cycloalkyl, spiro-C₄₋₇cycloalkenyl, spiro-heterocycloalkyl having 3-7 total ring atoms and 1 or 2 heteroatoms selected from N, O, and S, or spiro-heterocycloalkenyl having 4-7 total ring atoms and 1 or 2 heteroatoms selected from N, O, and S; or two vicinal R³, together with the atoms to which they are 3-7 total ring atoms and 1 or 2 heteroatoms selected from N, O, and S, or fused-heterocycloalkenyl having 4-7 total ring atoms and 1 or 2 heteroatoms selected from N, O, and S; and
R⁵ is halo, C₁₋₃haloalkyl, C₁₋₆alkyl, C₂₋₄alkenyl, C₂₋₄alkynyl, C₁₋₃alkoxy, C₁₋₃thioalkyl, C₃₋₇cycloalkyl, C₅₋₇cycloalkenyl, heterocycloalkyl having 3-7 total ring atoms and 1-3 heteroatoms selected from N, O, and S, or heterocycloalkenyl having 5-7 total ring atoms and 1-3 heteroatoms selected from N, O, and S, wherein each of the foregoing independently is unsubstituted or substituted with 1-3 substituents, and each substituent independently is C₁₋₃haloalkyl, C₀₋₃alkylene-OH, C₀₋₃alkylene-C₁₋₃alkoxy, Ca-cycloalkyl, C₅₋₇cycloalkenyl, heterocycloalkyl having 3-7 total ring atoms and 1-3 heteroatoms selected from N, O, and S, heterocycloalkenyl having 4-7 total ring atoms and 1-3 heteroatoms selected from N, O, and S, or phenyl; and
each R⁶ independently is Br, Cl, F. CN, CH₃, CH₂F, CHF₂, CF₃, OH, CH₂OH, OCH₃, OCD₃, CH₂OCH₃, or CH₂N(CH) 2, or two geminal R⁶, together with the atom to which they are attached, form oxo, =CH₂, spiro-cyclopropyl, spiro-cyclobutyl, spiro-oxetanyl, or spiro-tetrahydrofuranyl, or two vicinal R⁶, together with the atoms to which they are attached, form fused-cyclopropyl, fused-cyclobutyl, or fused-cyclopentyl, and any of the foregoing spiro and fused rings is unsubstituted or substituted with 1 or 2 substituents, and each substituent independently is halo, C₁₋₃alkyl, C₁₋₃haloalkyl, C₀₋₂alkyleneOH, C₀₋₂alkyleneC₁₋₃alkoxy, or C₀₋₂alkyleneCN.

In some cases, B is CH₂CH═CH, or CH₂CH₂OH; m is 0 or 1; o is 0 or 1; halo is Cl; [symbol] is

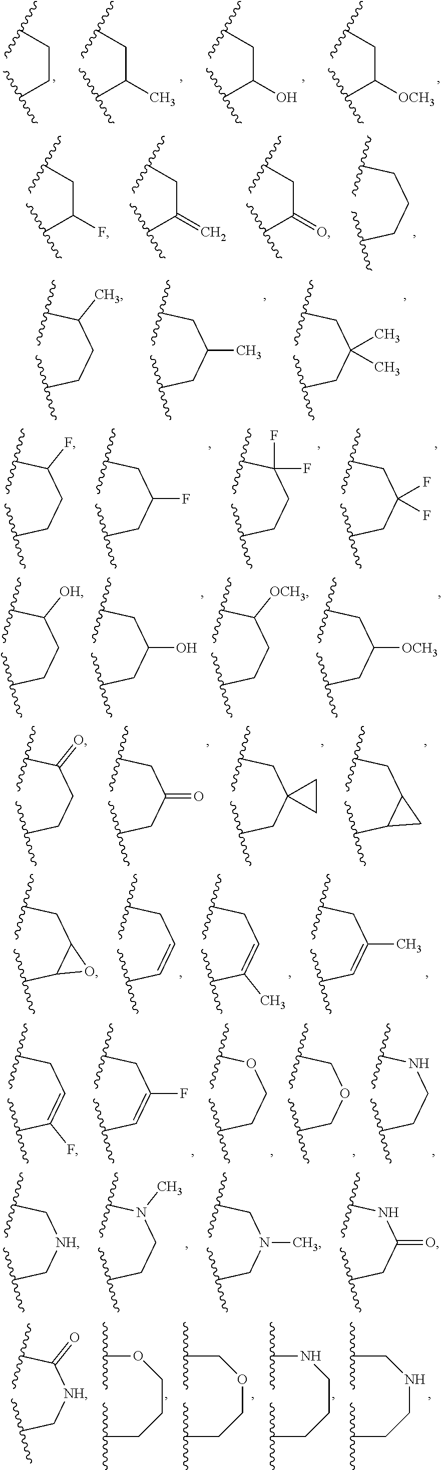

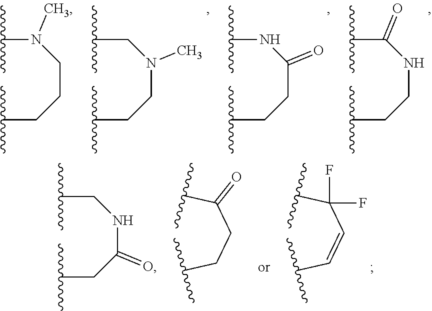

R³ is CH₃; R⁵ is CHF; or CF; R⁶ is CH₃; R^{ZA} is CH₃; and R^{ZB} is CH₃, C(CH₃)₂CH₂OH, CH₂CH₂OCH₃, CH₂CH₂OCD₃, CH(CH₃)OCH₃,

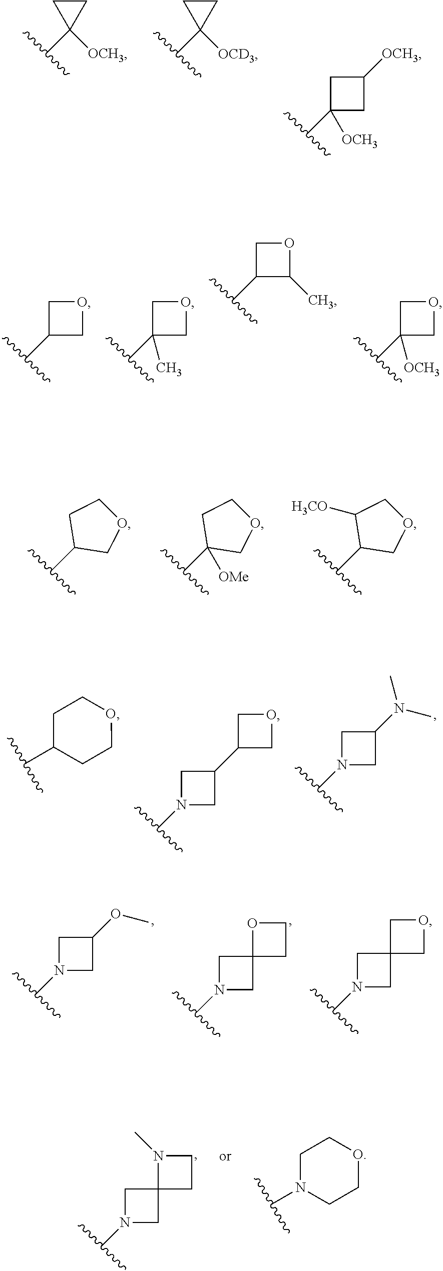

In some cases, the intermediate is a compound listed in Table INT-A, Table INT-A′, Table INT-B, Table INT-C, Table INT-D, Table INT-E, Table INT-F. Table INT, a nitrogen-protected analog of any of the foregoing, or a pharmaceutically acceptable salt of any of the foregoing.

Yet another aspect of the disclosure provides a process for preparing a compound described herein (e.g., a compound of Formula (I), Formula (I′), Formula (IA), Formula (IB), Formula (IE), Formula (IF), Formula (IG), Formula (II), Formula (II′), Formula (IIA), Formula (IIB), Formula (IIC), Formula (IID), Formula (IIE), and Formula (IIF), or a compound listed in Table A, Table A′, Table B, Table B′, and Table E), or a pharmaceutically acceptable salt of any of the foregoing comprising converting an intermediate described herein, such as an intermediate of Formula (Int-AA), Formula (Int-AB), Formula (Int-AC), Formula (Int-AD), Formula (Int-AE), Formula (Int-AF), Formula (Int-AG), Formula (Int-AH), Formula (Int-AI), Formula (Int-AJ), Formula (Int-B), Formula (Int-C), Formula (Int-D), and Formula (Int-E), or an intermediate listed in Table INT-A, Table INT-A′, Table INT-B, Table INT-C. Table INT-D. Table INT-E, Table INT-F, or Table INT, a nitrogen-protected analog thereof, or a pharmaceutically acceptable salt thereof, into a compound of the disclosure (e.g., a compound of Formula (I), Formula (I′), Formula (IA), Formula (IB), Formula (IE), Formula (IF), Formula (IG), Formula (II), Formula (II′), Formula (IIA), Formula (IIB), Formula (IIC), Formula (IID), Formula (IIE), and Formula (IIF), or a compound listed in Table A, Table A′, Table B, Table B′, and Table E), or a pharmaceutically acceptable salt of any of the foregoing.

Further aspects and advantages will be apparent to those of ordinary skill in the art from a review of the following detailed description. The description hereafter includes specific cases, embodiments, and examples with the understanding that the disclosure is illustrative and is not intended to limit the embodiments of the present disclosure to the specific cases, embodiments, and examples described herein.

### DETAILED DESCRIPTION

Disclosed herein are compounds having activity as inhibitors of the G12C-mutant KRAS protein, pharmaceutical compositions comprising the compounds, and uses and methods of treating disorders, such as cancer, with the compounds and pharmaceutical composition described herein.

### Compounds of Formula (II)

Provided herein are compounds of Formula (II):

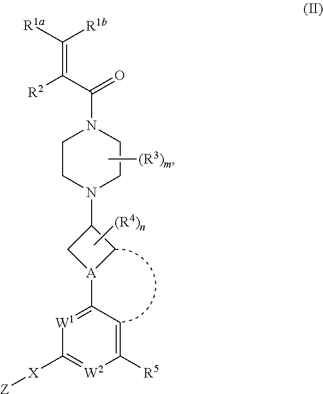

and pharmaceutically acceptable salts thereof, wherein:
m is 0, 1, 2, 3, or 4;
n is 0, 1, or 2;
A is N, CH, C-halo, C—CN, C—C₁₋₃alkyl, C₁₋₃haloalkyl, C—C₀₋₃alkyleneOH, or C—C₀₋₃alkylene-C₁₋₄alkoxy;
each of W¹ and W² independently is N, CH, C-halo, C—CN, C—C₁₋₃alkyl, C—C₂₋₆alkenyl, C—C₂₋₃alkynyl, C—C₁₋₃haloalkyl, C—C₀₋₃alkyleneOH, or C—C₀₋₃alkylene-C₁₋₄alkoxy, wherein each of the alkenyl and alkynyl is unsubstituted or substituted with 1 or more substituents;
X is heterocycloalkyl or heterocycloalkenyl, each having 4-7 total ring atoms and 1-3 heteroatoms selected from N, O, and S, wherein each of the heterocycloalkyl and heterocycloalkenyl is unsubstituted or substituted with 1 or more substituents;
Z is phenyl, heteroaryl having 5 or 6 total ring atoms and 1-3 heteroatoms selected from N, O, and S, or a bicyclic ring comprising a heteroaryl having 5 or 6 total ring atoms and 1-3 heteroatoms selected from N, O, and S fused to C₅₋₆cycloalkyl or a heterocycloalkyl ring having 5 or 6 total ring atoms and 1 or 2 heteroatoms selected from N, O, and S; wherein each of the phenyl, heteroaryl, and bicyclic ring is unsubstituted or substituted with 1 or more substituents;
[symbol] is C₂₋₆alkylene, C₃₋₆alkenylene, heteroalkylene having 2-6 total atoms and 1-3 heteroatoms selected from N, O, and S, or heteroalkenylene having 3-6 total atoms and 1 or 2 heteroatoms selected from N, O, and S, wherein is [symbol] unsubstituted or substituted with 1 or more substituents;
each of R^{1a}, R^{1b}, and R¹ independently is H, D, halo, C₁₋₄alkyl, C₁₋₄haloalkyl, C₁₋₂alkylene-OH, C₀₋₂alkylene-C₁₋₄alkoxy, C₀₋₂alkylene-C₁₋₄haloalkoxy, C₀₋₂alkylene-CN, C₀₋₂alkylene-N(R^{N1})₂, C₁₋₂alkylene-heterocycloalkyl having 3-6 total ring atoms and 1 or 2 heteroatoms selected from N, O, and S, or R^{1b} and R², together with the carbon atoms to which they are attached, form a

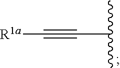

each R³ independently is C₁₋₃alkyl, C₁₋₃haloalkyl,

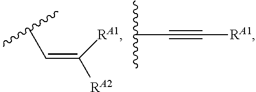

C₀₋₃alkyleneCN, C₀₋₃alkyleneOH, or C₀₋₃alkylene-C₁₋₄alkoxy; two geminal R³, together with the atom to which they are attached, form oxo, spiro-C₃₋₇cycloalkyl, spiro-C₄₋₇cycloalkenyl, spiro-heterocycloalkyl having 3-7 total ring atoms and 1 or 2 heteroatoms selected from N, O, and S, or spiro-heterocycloalkenyl having 4-7 total ring atoms and 1 or 2 heteroatoms selected from N, O, and S; or two vicinal R³, together with the atoms to which they are attached, form fused-C₃₋₇cycloalkyl, fused-C₄₋₇cycloalkenyl, fused-heterocycloalkyl having 3-7 total ring atoms and 1 or 2 heteroatoms selected from N, O, and S, or fused-heterocycloalkenyl having 4-7 total ring atoms and 1 or 2 heteroatoms selected from N, O, and S;
each R⁴ independently is C₁₋₃alkyl, C₁₋₄haloalkyl, C₀₋₂alkyleneCN, C₀₋₃alkyleneOH, or C₁₋₃alkylene-C₁₋₃alkoxy; or two geminal R⁴, together with the atom to which they are attached, form oxo, spiro-C₃₋₇cycloalkyl, or spiro-heterocycloalkyl having 4-7 total ring atoms and 1 or 2 heteroatoms selected from N, O and S;
R⁵ is halo, C₁₋₃haloalkyl, C₁₋₆alkyl, C₂₋₄alkenyl, C₂₋₄alkynyl, C₁₋₃alkoxy, C₁₋₃thioalkyl, C₃₋₇cycloalkyl, C₅₋₇cycloalkenyl, heterocycloalkyl having 3-7 total ring atoms and 1-3 heteroatoms selected from N, O, and S, or heterocycloalkenyl having 5-7 total ring atoms and 1-3 heteroatoms selected from N, O, and S, wherein each of the foregoing is independently unsubstituted or substituted with 1 or more substituents;
each of R^{A1} and R^{A2} independently is H, C₁₋₃alkyl, C₁₋₃haloalkyl, or C₃₋₅cycloalkyl; and
each R^{N1} independently is H or C₁₋₄alkyl.

In some cases, R^{3a} is H or D. In some cases, R^{1a} is H. In some cases, R^{3a} is D. In some cases, R^{1b} is H or D. In some cases, R^{1b} is H. In some cases, R^{1b} is D. In some cases, R² is H or D. In some cases, R² is H. In some cases, R² is D. In some cases, at least one of R^{1a}, R^{1b}, and R² is H or D. In some cases, at least one of R^{1a}, R^{1b}, and R² is H. In some cases, at least one of R^{1a}, R^{1b}, and R² is D. In some cases, at least two of R^{1a}, R^{1b}, and R² are each independently H or D. In some cases, at least two of R^{1a}, R^{1b}, and R² are H. In some cases, at least two of R^{1a}, R^{1b}, and R² are D. In some cases, each of R^{1a}, R^{1b}, and R² independently is H or D. In some cases, two of R^{1a}, R^{1b}, and R² are H and one of R^{1a}, R^{1b}, and R² is halo, CH₄alkyl, C₁₋₄haloalkyl, C₁₋₂alkylene-OH, C₀₋₂alkylene-C₁₋₃alkoxy, C₀₋₂alkylene-C₁₋₄haloalkoxy, C₀₋₂alkylene-CN, C₀₋₂alkylene-N(R^{N1})₂, or C₁₋₂alkylene-heterocycloalkyl having 3-6 total ring atoms and 1 or 2 heteroatoms selected from N, O, and S. In some cases, each of R^{1a}, R^{1b}, and R² is H. In some cases, each of R^{1a}, R^{1b}, and R² is D. In some cases, at least one of R^{1a}, R^{1b}, and R² is halo. In some cases, one of R^{1a}, R^{1b}, and R² is halo. In some cases, R^{1a} is halo and each of R^{1b} and R² is H. In some cases, at least one of R^{1a}, R^{1b}, and R² is Br, Cl, or F. In some cases, one of Ria, R^{1b}, and R² is Br, Cl, or F. In some cases, R^{1a} is Br, Cl, or F and each of R^{1b} and R² is H. In some cases, at least one of R^{1a}, R^{1b}, and R² is Br or Cl. In some cases, one of R^{1a}, R^{1b}, and R² is Br or Cl. In some cases, R^{1a} is Br or Cl and each of R^{1a} and R² is H. In some cases, at least one of R^{1a}, R^{1b}, and R² is C₁₋₄alkyl or C₁₋₃haloalkyl. In some cases, one of R^{1a}, R^{1b}, and R² is C₁₋₄alkyl or C₁₋₃haloalkyl. In some cases, at least one of R^{1a}, R^{1b}, and R² is CH₃, CH₂CH₃, CH₂CH₂CH₃, CH(CH₃)₃, CH₂CH₂CH—CH₃, CH₂F, CHF₂, or CF₃. In some cases, one of R^{1a}, R^{1b}, and R² is CH₃, CH₂CH₃, CH₂CHCH₃, CH(CH₃)₂, CH₂CH₂CH₂CH₃, CH₂F, CHF₂, or CF₃. In some cases, at least one of R^{1a}, R^{1b}, and R² is CH₃, CH₂F, CHF₂, or CF₃. In some cases, one of R^{1a}, R^{1b}, and R² is CH₃, CH₂F, CHF₂, or CF₃. In some cases, at least one of R^{1a}, R^{1b}, and R² is C₁₋₂alkylene-OH, C₀₋₂alkylene-C₁₋₄alkoxy, C₀₋₂alkylene-C₁₋₄haloalkoxy, C₀₋₂alkylene-CN, or C₀₋₂alkylene-N(R^{N1})₂, and each R^{N1} independently is H or C₁₋₄alkyl. In some cases, each R^{N1} independently is H or CH₃. In some cases, each R^{N1} independently is H. In some cases, at least one of R^{1a}, R^{1b}, and R² is CH₂OH, OCH₃, CH₂OCH₃, OCF₃, CH₂OCF₃, CN, CH₂CN, NH₂, N(CH₃)₂, CH₂NH₂, or CH₂N(CH₃)₂. In some cases, one of R^{1a}, R^{1b}, and R² is CH₂OH, OCH₃, CH₂OCH₃, OCF₃, CH₂OCF₃, CN, CH₂CN, NH₂, N(CH₃)₂, CH₂NH₂, or CH₂N(CH₃)₂. In some cases, at least one of R^{1a}, R^{1b}, and R² is C₁₋₂alkylene-heterocycloalkyl wherein the heterocycloalkyl contains 3-6 total ring atoms and 1 or 2 heteroatoms selected from N, O, and S. In some cases, the heterocycloalkyl is aziridinyl, oxiranyl, azetidinyl, oxetanyl, pyrrolidinyl, tetrahydrofuranyl, imidazolidinyl, pyrazolidinyl, oxathiolidinyl, isoxthiodinyl, oxazolidinyl, isoxazolidinyl, thiazolidinyl, isothiazolidinyl, piperidinyl, diazinyl, or morpholinyl. In some cases, the heterocycloalkyl is aziridinyl, azetidinyl, pyrrolidinyl, piperidinyl, or morpholinyl. In some cases, at least one of R^{1a}, R^{1b}, and R² is aziridin-1-yl-methyl, azetidin-1-yl-methyl, pyrrolidine-1-yl-methyl, piperidin-1-yl-methyl, or morpholin-1-yl-methyl. In some cases, one of R^{1a}, R^{1b}, and R² is aziridin-1-yl-methyl, azetidin-1-yl-methyl, pyrrolidine-1-yl-methyl, piperidin-1-yl-methyl, or morpholin-1-yl-methyl. In some cases, one of R^{1a}, R^{1b}, and R² is Br, Cl, F, CH₃, CH₂F, CHF₂, CF₃, CH₂OH, OCH₃, CH₂OCH₃, OCF₃, CH₂OCF₃, CN, CH₂CN, NH₂, N(CH₃)₂, CH₂NH₂, CH₂N(CH₃)₂, aziridin-1-yl-methyl, azetidin-1-yl-methyl, pyrrolidine-1-yl-methyl, piperidin-1-yl-methyl, or morpholin-1-yl-methyl. In some cases, R″ and R′, together with the carbon atoms to which they are attached, form

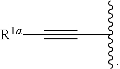

In some cases, R^{1a} is H. In some cases, R^{1b} and R², together with the carbon atoms to which they are attached, form

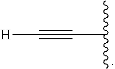 [SMILES: *C#C]

In some cases,

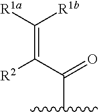

is

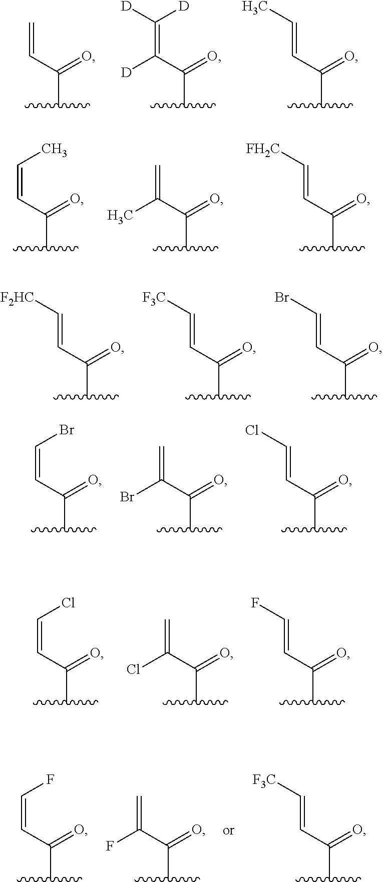

In some cases,

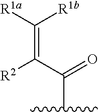

is

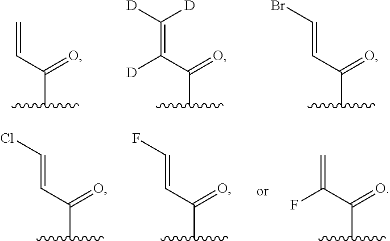

In some cases,

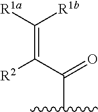

is

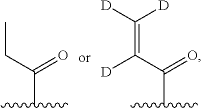

In some cases,

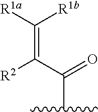

is

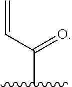 [SMILES: *C(=O)C=C]

In some cases, m is 0, and

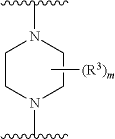

is

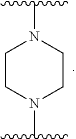 [SMILES: *N1CCN(*)CC1]

In some cases, m is 1. In some cases, m is 2. In some cases, m is 3. In some cases, m is 4. In some cases,

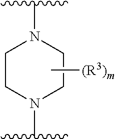

is deuterated. In some cases,

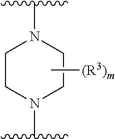

is fully deuterated. In some cases,

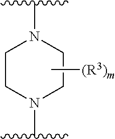

is

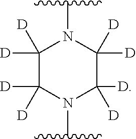

In some cases, at least one R³ is C₁₋₃alkyl or C₁₋₃haloalkyl. In some cases, at least one R³ is CH₃, CH₂CH₃, CH₂CH₂CH₃, CH(CH₃)₂, CF₃, CHF₂, or CH₂F. In some cases, at least one R³ is CH₃, CH₂CH₃, CF₃; CHF₂, or CH₂F. In some cases, at least one R³ is CH₃. In some cases, m is 1 or 2 and each R³ is CH₃. In some cases, m is 1 and R³ is CF₃, CHF₂, or CH₂F. In some cases, at least one R³ is

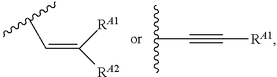

and each of R^{A1} and R^{A2} independently is H, C₁₋₃alkyl, C₁₋₃haloalkyl, or C₃₋₅cycloalkyl. In some cases, m is 1 and R³ is

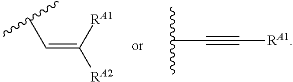

is some cases, each of R^{A1} and R^{A2} independently is H, CH₃, CH₂F, CHF₂, CF₃, CH₂CH₃, CH₂CH₂CH₃, CH(CH₃)₂, cyclopropyl, or cyclobutyl. In some cases,

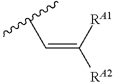

is

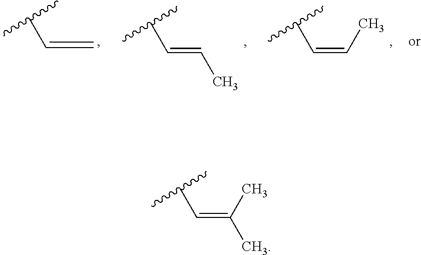

In some cases,

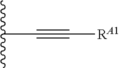

is

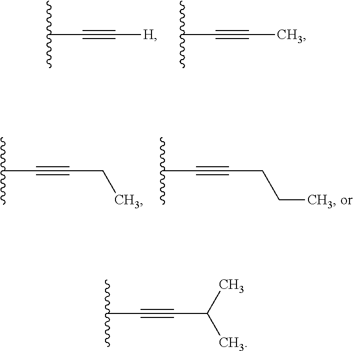

In some cases,

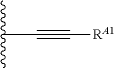

is

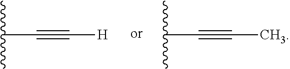

In some cases, at least one R³ is

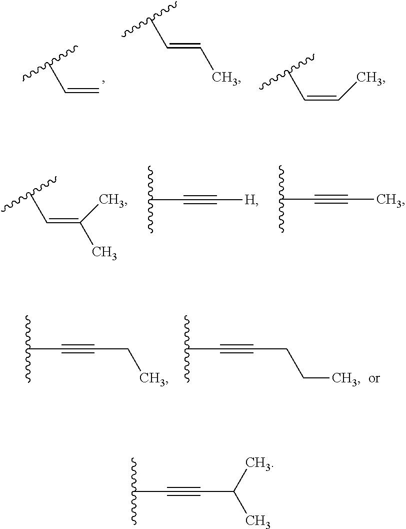

In some cases, at least one R³ is

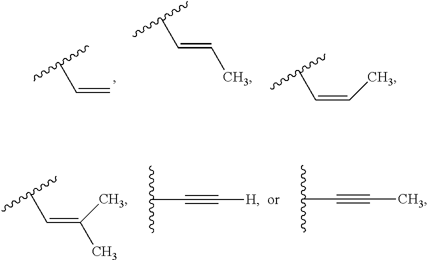

In some cases, at least one R³ is C₀₋₃alkyleneCN, In some cases, at least one R³ is CN, CH₂CN, or CH₂CH₂CN. In some cases, at least one R³ is CN or CH₂CN. In some cases, m is 1 and R′ is CN or CH₂CN. In some cases, at least one R³ is C₀₋₃alkyleneOH or C₀₋₆alkylene-C₁₋₃alkoxy. In some cases, at least one R³ is OH, CH₂OH, CH₂CH₂OH, OCH₃, CH₂OCH₃, or CH₂CH₂OCH₃. In some cases, m is 1 and R³ is OH, CH₂OH, CH₂CH₂OH, OCH₃, CH₂OCH₃, or CH₂CH₂OCH₃. In some cases, two geminal R³, together with the atom to which they are attached, form oxo (═O). In some cases, two geminal R³, together with the atom to which they are attached, form C₃₋₇spiro-cycloalkyl or spiro-heterocycloalkyl having 3-7 total ring atoms and 1 or 2 heteroatoms selected from N, O and S. In some cases, the spiro-cycloalkyl is spiro-cyclopropyl, spiro-cyclobutyl, or spiro-cyclopentyl. In some cases, the spiro-heterocycloalkyl is spiro-azetidinyl, spiro-oxetanyl, spiro-pyrrolidinyl, spiro-imidazolidinyl, spiro-pyrazolidinyl, or spiro-tetrahydrofuranyl. In some cases, two geminal R³, together with the atom to which they are attached, form spiro-cyclopropyl, spiro-cyclobutyl, spiro-cyclopentyl, spiro-azetidinyl, spiro-oxetanyl, spiro-pyrrolidinyl, spiro-imidazolidinyl, spiro-pyrazolidinyl, or spiro-tetrahydrofuranyl. In some cases, two geminal R³, together with the atom to which they are attached, form spiro-C₄₋₇cycloalkenyl or spiro-heterocycloalkenyl having 4-7 total ring atoms and 1 or 2 heteroatoms selected from N, O and S. In some cases, two vicinal R³, together with the atoms to which they are attached, form fused-C₃₋₇cycloalkyl or fused-heterocycloalkyl having 3-7 total ring atoms and 1 or 2 heteroatoms selected from N, O and S. In some cases, two vicinal R³, together with the atoms to which they are attached, form fused-cyclopropyl, fused-cyclobutyl, fused-cyclopentyl, or fused-cyclohexyl. In some cases, two vicinal R³, together with the atoms to which they are attached, form fused-cyclopropyl or fused-cyclobutyl. In some cases, each R³ independently is CH₃, CH₂CH₃, CF₃, CHF₂, CH₂F,

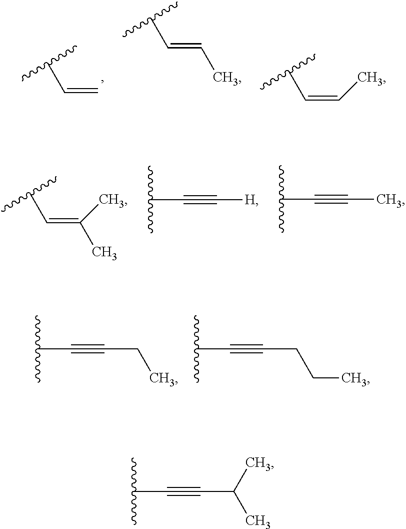

CN, CH₂CN, OH, CH₂OH, CH₂CH₂OH, OCH₃, CH₂OCH₃, or CH₂CH₂OCH₃, two geminal R³, together with the atom to which they are attached, form oxo (═O), spiro-cyclopropyl, spiro-cyclobutyl, spiro-oxetanyl, or spiro-tetrahydrofuranyl; or two vicinal R³, together with the atoms to which they are attached, form fused-cyclopropyl or fused-cyclobutyl. In some cases, m is 0; or m is 1 and R³ is CH₃, CH₂F, CHF₂, CF₃, CN, CH₂CN, CH₂OH, or CH₂OCH₃; or m is 2 and two geminal R³, together with the atom to which they are attached, form spiro-oxetanyl. In some cases, m is 0; or m is 1 and R³ is CH₃. In some cases,

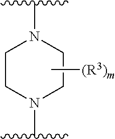

is

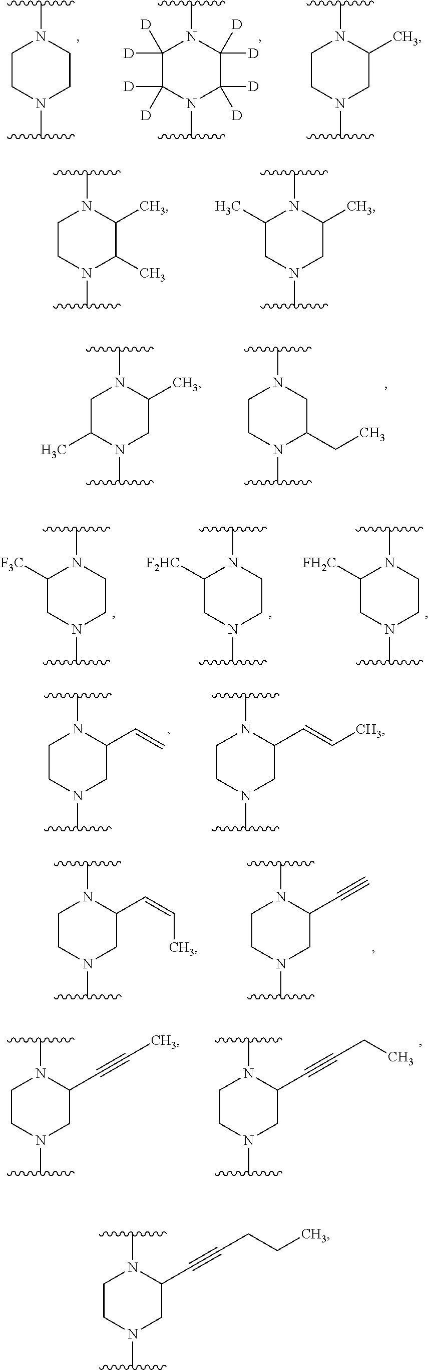

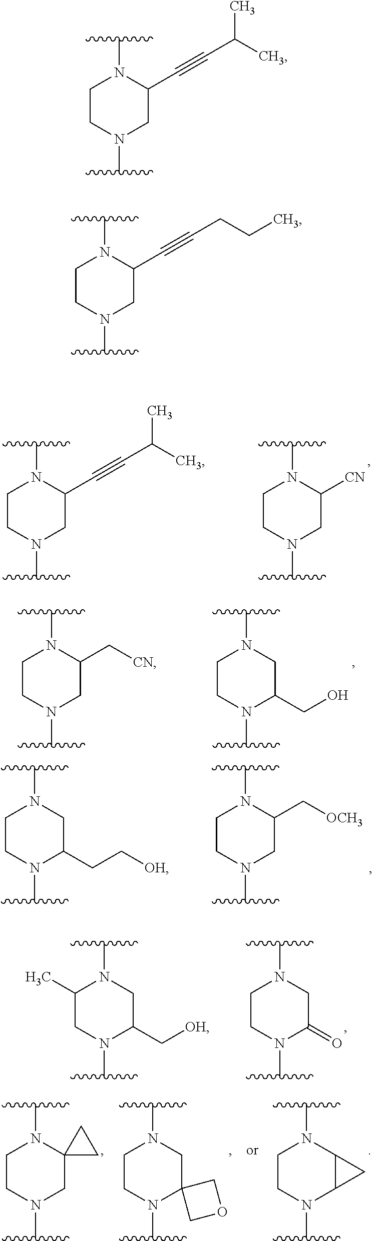

In some cases,

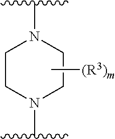

is

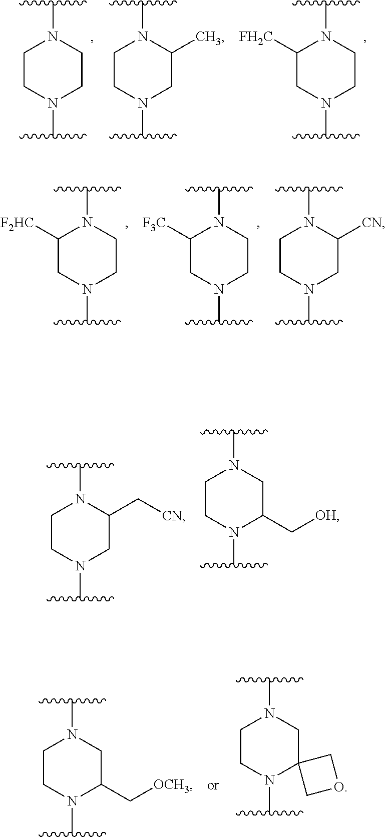

In some cases,

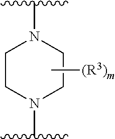

is

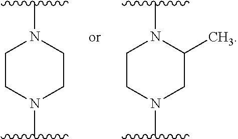 [SMILES: *N1CCN(*)CC1 ; *N1CCN(*)C(C)C1]

In some cases, A is N, CH, or C—C₁₋₃alkyl. In some cases, A is N. In some cases, A is CH, C-halo, C—CN, C—C₁₋₃alkyl, C—C₁₋₃haloalkyl, C—C₀₋₂alkyleneOH, or C₀₋₃alkylene-C₁₋₃alkoxy. In some cases, A is CH. In some cases, A is C—F, C—Cl, or C—CN. In some cases. A is C-halo or C—CN. In some cases, A is C—F or C—Cl. In some cases, A is C—F. In some cases, A is C—CN. In some cases, A is C—C₁₋₃alkyl or C—C₁₋₃haloalkyl. In some cases, A is C—CH₃, C—CH₂CH₃, C—CH₂CH₂CH₃, C—CH(CH₃)₂, C—CF₃, C—CHF₂, or C—CH₂F. In some cases, A is C—CH₃, C—CH₂F, C—CHF₂, or C—CF₃. In some cases, A is C—CH₃. In some cases, A is C—CH₂F, C—CHF₂, or C—CF₃. In some cases, A is C—C₀₋₃alkyleneOH or C—C₀₋₃alkylene-C₁₋₄alkoxy. In some cases, A is C—OH, C—CH₂OH, C—CH₂CH₂OH, C—OCH₃, C—CH₂OCH₃, or C—CH₂CH₂OCH₃. In some cases, A is C—OH, C—CH₂OH, C—OCH₃, or C—CH₂OCH₃. In some cases, A is CH. C—F, C—Cl, C—CN, C—CH₃, C—CH₂F, C—CHF₂, C—CF₃, C—OH, C—CH₂OH, C—OCH₃, or C—CH₂OCH₃. In some cases, A is N, CH, C—F, C—Cl, C—CN, C—CH₃, C—CH₂CH₃, C—CH₂CH₂CH₃, C—CH(CH₃)₂, C—CF₃, C—CHF₂, C—CH₂F, C—OH, C—CH₂OH, C—CH₂CH₂OH, C—OCH₃, C—CH₂OCH₃, or C₂CH₂CH₂OCH₃. In some cases, A is N, CH, C—F, C—Cl, C—CN, C—CH₃, C—CF₃, C—CHF₂, C—CH₂F, C—OH, C—CH₂OH, C—OCH₃, or C—CH₂OCH₃. In some cases, A is N, CH, or C₂CH₃. In some cases, n is 0. In some cases, n is 1. In some cases, n is 2. In some cases, at least one R⁴ is C₁₋₃alkyl or C₁₋₃haloalkyl. In some cases, at least one R⁴ is CH₃, CH₂CH₃, CH₂CH₂CH₃, CH(CH₃)₂, CF₃, CHF₂, or CH₂F. In some cases, at least one R⁴ is CH₃. In some cases, one R⁴ is CH₃, CH₂CH₃, CH₂CH₂CH₃, CH(CH₃)₂, CF₃, CHF₂, or CH₂F. In some cases, n is 2 and each R⁴ independently is CH₃, CH₂CH₃, CH₂CH₂CH₃, CH(CH₃)₂, CF₃, CHF₂, or CH₂F. In some cases, n is 1 and R⁴ is CH₃, CH₂CH₃, CH₂CH₂CH₃, CH(CH₃)₂, CF₃, CHF₂, or CH₂F. In some cases, n is 1 and R⁴ is CH₃. In some cases, at least one R⁴ is C₀₋₃alkyleneCN. In some cases, at least one R⁴ is CN or CH₂CN, In some cases, n is 1 and R⁴ is CN or CH₂CN. In some cases, at least one R⁴ is C₁₋₃alkyleneOH or C₁₋₃alkylene-C₁₋₃alkoxy. In some cases, at least one R⁴ is CH₂OH, CH₂CH₂OH, OCH₃, CH₂OCH₃, or CH₂CH₂OCH₃. In some cases, n is 1 and R⁴ is CH₂OH, CH₂CH₂OH, OCH₃, CH₂OCH₃, or CH₂CH₂OCH₃. In some cases, two geminal R⁴, together with the atom to which they are attached, form oxo (═O). In some cases, two geminal Rf, together with the atom to which they are attached, form C₃₋₇spiro-cycloalkyl. In some cases, the spiro-cycloalkyl is spiro-cyclopropyl, spiro-cyclobutyl, or spiro-cyclopentyl. In some cases, the spiro-cycloalkyl is spiro-cyclopropyl or spiro-cyclobutyl. In some cases, two geminal R⁴, together with the atom to which they are attached, form spiro-heterocycloalkyl having 4-7 total ring atoms and 1 or 2 heteroatoms selected from N, O and S. In some cases, the spiro-heterocycloalkyl is spiro-oxetanyl or spiro-tetrahydrofuranyl. In some cases, the spiro-heterocycloalkyl is spiro-oxetanyl. In some cases, two geminal R⁴, together with the atom to which they are attached, form spiro-cyclopropyl, spiro-cyclobutyl, or spiro-oxetanyl. In some cases, each R⁴ independently is CH₃, CH₂CH₃, CH₂CH₂CH₃, CH(CH₃)₂, CF₃, CHF₂, CH₂F, CN, CH₂CN, CH₂OH, CH₂CH₂OH, OCH₃, CH₂OCH₃, CH₂CH₂OCH₃; or two geminal R⁴, together with the atom to which they are attached, form oxo, spiro-cyclopropyl, spiro-cyclobutyl, or spiro-oxetanyl. In some cases, each R⁴ independently is CH₃, CH₂CH₃, CH₂CH₂CH₃, CH₂F, CN, CH₂CN, CH₂OH, CH₂CH₂OH, CH₂OCH₃, or two geminal R⁴, together with the atom to which they are attached, form spiro-cyclopropyl. In some cases, each R⁴ independently is CH₃, CH₂CH₃, CH₂CH₂CH₃, CH(CH₃)₂, CF₃, CHF₂, CH₂F, CN, CH₂CN, CH₂OH, CH₂CH₂OH, CH₂OCH₃, CH₂CH₂OCH₃, or two geminal R⁴, together with the atom to which they are attached, form oxo, spiro-cyclopropyl, spiro-cyclobutyl, or spiro-oxetanyl. In some cases,

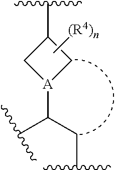

is

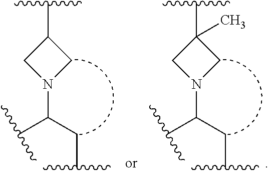

In some cases,
is

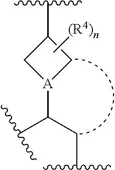

is

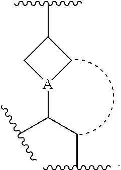

In some cases,

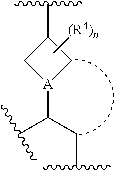

is

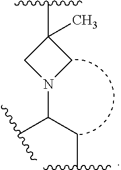 [SMILES: *CC(*)N1CC(*)(C)C1]

In some cases, [symbol] is unsubstituted. In some cases, [symbol] is substituted with 1-4 substituents. In some cases, each of the 1-4 substituents independently is C₁₋₃alkyl, C₁₋₃haloalkyl, C₂₋₃alkenyl, halo, CN, C₀₋₃alkyleneOH, C₀₋₂alkylene-C₁₋₃alkoxy, C₃₋₅cycloalkyl, C₄₋₅cycloalkenyl, heterocycloalkyl having 4 or 5 total ring atoms and 1-3 heteroatoms selected from N, O, and S, heterocycloalkenyl having 4 or 5 total ring atoms and 1-3 heteroatoms selected from N, O, and S, phenyl; or two geminal substituents, together with the atom to which they are attached, form oxo, ═CH₂, spiro-C₃₋₅cycloalkyl, spiro-C₄₋₅cycloalkenyl, spiro-heterocycloalkyl having 3-5 total ring atoms and 1 or 2 heteroatoms selected from N, O and S, or spiro-heterocycloalkenyl having 4 or 5 total ring atoms and 1 or 2 heteroatoms selected from N, O and S; or two vicinal substituents, together with the atoms to which they are attached, form fused-C₃₋₅cycloalkyl, fused-C₄₋₅cycloalkenyl, fused-heterocycloalkyl having 3-5 total ring atoms and 1 or 2 heteroatoms selected from N, O and S or fused-heterocycloalkenyl having 4 or 5 total ring atoms and 1 or 2 heteroatoms selected from N, O and S. In some cases, each substituent independently is C₁₋₃alkyl, C₁₋₃haloalkyl. C₂₋₃alkenyl, halo, C₀₋₃alkyleneOH, C₀₋₆alkylene-C₁₋₃alkoxy; or two geminal substituents, together with the atom to which they are attached, form oxo or ═CH₂; or two vicinal substituents, together with the atoms to which they are attached, form fused-C₃₋₅cycloalkyl or fused-heterocycloalkyl having 4 or 5 total ring atoms and 1 or 2 heteroatoms selected from N, O and S. In some cases, each C₁₋₃alkyl substituent independently is CH₃, CH₂CH₃, CH₂CH₂CH₃, or CH(CH₃)₂. In some cases, each C₁₋₃alkyl substituent is CH₃. In some cases, each C₁₋₃haloalkyl substituent independently is CF₃, CHF₂, or CH₂F. In some cases, each C₂₋₆alkenyl substituent independently is CH═CH₂, CH═CHCH₃, or CH₂CH═CH₂. In some cases, each halo substituent independently is Cl or F. In some cases, each C₀₋₃alkyleneOH substituent independently is OH, CH₂OH, or CH₂CH₂OH. In some cases, each C₀₋₃alkylene-C₁₋₃alkoxy substituent independently is OCH₃, OCH₂CH₃, CH₂OCH₃, or CH₂OCH₂CH₃. In some cases, each C₃₋₅cycloalkyl substituent independently is cyclopropyl, cyclobutyl, or cyclopentyl. In some cases, each C₄₋₅cycloalkenyl substituent independently is cyclobutenyl or cyclopentenyl. In some cases, each heterocycloalkyl substituent independently is oxetanyl, tetrahydrofuranyl, aziridinyl, or azetidinyl. In some cases, each spiro-cycloalkyl substituent independently is spiro-cyclopropyl or spiro-cyclobutyl. In some cases, each spiro-cycloalkenyl is spiro-cyclobutenyl. In some cases, each spiro-heterocycloalkyl independently is spiro-oxetanyl, spiro-tetrahydrofuranyl, spiro-aziridinyl, or spiro-azetidinyl. In some cases, each fused-cycloalkyl substituent independently is fused-cyclopropyl or fused-cyclobutyl. In some cases, each fused-cycloalkenyl is fused-cyclobutenyl. In some cases, each fused-heterocycloalkyl independently is fused-oxetanyl, fused-tetrahydrofuranyl, fused-aziridinyl, or fused-azetidinyl. In some cases, each of the 1-4 substituents of [symbol] independently is CH₃, CH₂CH₃, CH₂CH₂CH₃, CH(CH₃)₂, CF₃, CHF₂, CH₂F, CH═CH₃, CH—CHCH₃, CH₂CH═CH₂, Cl, F, OH, CH: OH, CH₂CH₂OH, OCH₃, OCH₂CH₃, CH₂OCH₃, CH₂OCH₂CH₃, cyclopropyl, cyclobutyl, oxetanyl, tetrahydrofuranyl, aziridinyl, or azetidinyl; or two geminal substituents, together with the atom to which they are attached, form spiro-cyclopropyl, spiro-cyclobutyl, spiro-oxetanyl, spiro-tetrahydrofuranyl, spiro-aziridinyl, or spiro-azetidinyl; or two vicinal substituents, together with the atoms to which they are attached form fused-cyclopropyl, fused-cyclobutyl, fused-oxetanyl, fused-tetrahydrofuranyl, fused-aziridinyl, or fused-azetidinyl. In some cases, each of the 1-4 substituents of [symbol] independently is CH₃, =CH₂, oxo, Cl, F, OH, OCH₃,

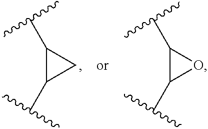 [SMILES: *C1CC1* ; *C1OC1*]

In some cases, [symbol] is C₂₋₆alkylene, wherein the C₂₋₆alkylene is unsubstituted or substituted with 1-4 substituents. In some cases, [symbol] is C₂alkylene, wherein the C₂alkylene is unsubstituted or substituted with 1-4 substituents. In some cases, [symbol] is

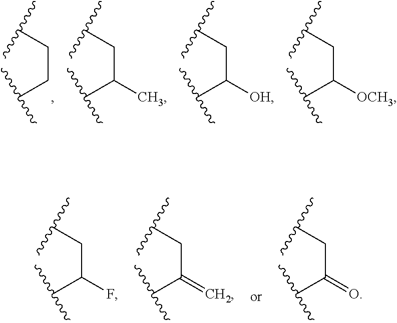

In some cases, [symbol] is C₃alkylene, wherein the C₃alkylene is unsubstituted or substituted with 1-4 substituents. In some cases, [symbol] is

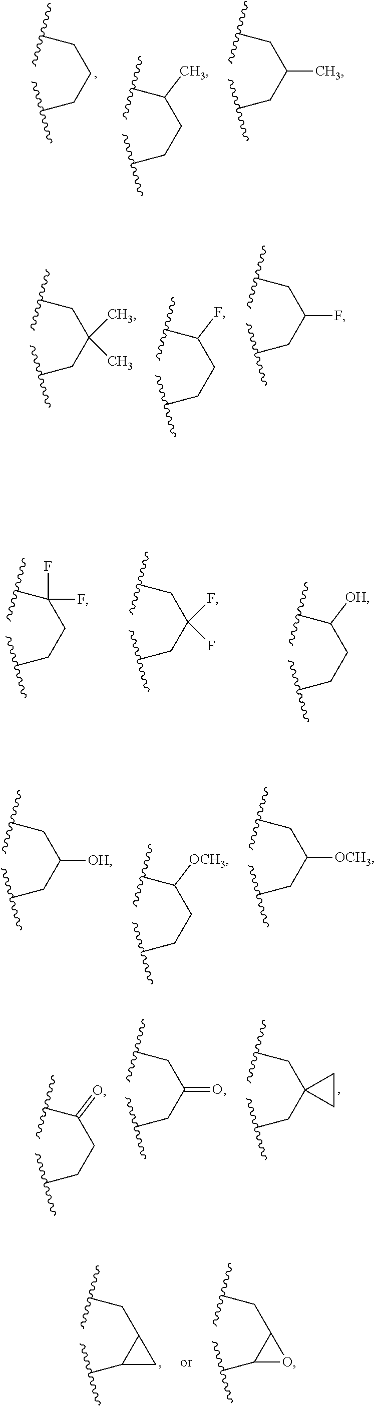

In some cases, [symbol] is C₄₋₆alkylene, wherein the C₄₋₆alkylene is unsubstituted or substituted with 1-4 substituents. In some cases, [symbol] is

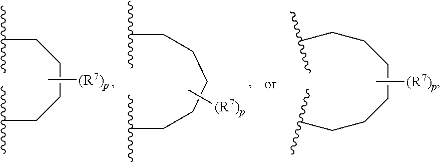

wherein p is 0, 1, 2, or 3, and each R⁷ independently is CH₃, Cl, F, OH, or OCH₃; or two geminal R⁷, together with the atom to which they are attached form oxo or ═CH₂; or two vicinal R⁷, together with the atoms to which they are attached form

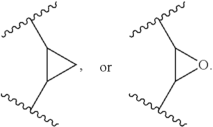 [SMILES: *C1CC1* ; *C1OC1*]

In some cases, [symbol] is

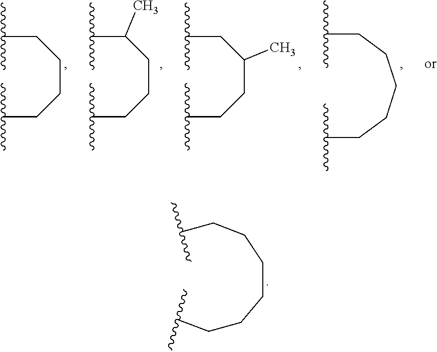

In some cases, [symbol] is C₃₋₆alkenylene, wherein the C₃₋₆alkenylene is unsubstituted or substituted with 1-4 substituents. In some cases, [symbol] is C₃alkenylene, wherein the C₃alkenylene is unsubstituted or substituted with 1-4 substituents. In some cases, [symbol] is

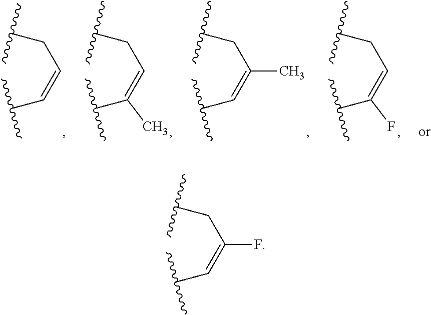

In some cases, [symbol] is C₂₋₆alkenylene, wherein the C₄₋₆alkenylene is unsubstituted or substituted with 1-4 substituents. In some cases, [symbol] is

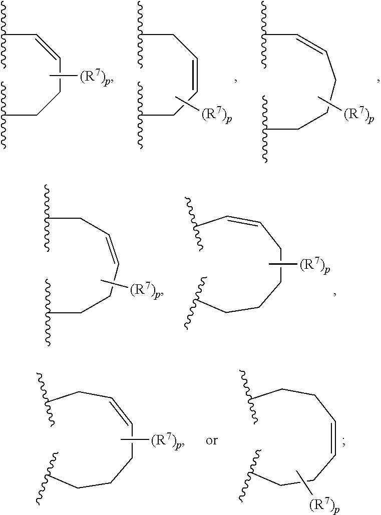

wherein p is 0, 1, 2, or 3, and each R⁷ independently is CH₃, Cl, F, OH, OCH₃,

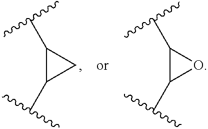 [SMILES: *C1CC1* ; *C1OC1*]

In some cases, [symbol] is heteroalkylene having 2-6 total atoms and 1-3 heteroatoms selected from N, O, and S. In some cases, the heteroalkylene has 2-4 total atoms and 1 or 2 heteroatoms selected from N, O, and S. In some cases, [symbol] is heteroalkylene having 2 total atoms and 1 heteroatom selected from N, O, and S. In some cases, [symbol] is

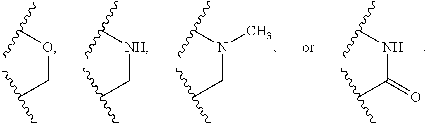

In some cases, the heteroalkylene has 3 total atoms and 1 heteroatom selected from N, O, and S. In some cases, [symbol] is

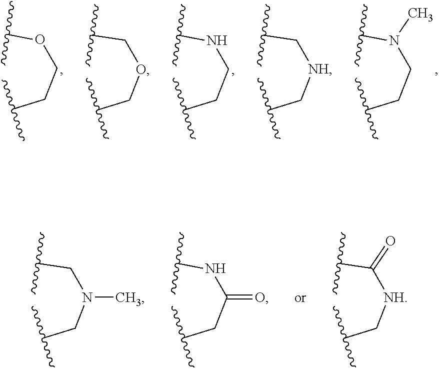

In some cases, the heteroalkylene has 4 total atoms and 1 heteroatom selected from N, O, and S. In some cases, the

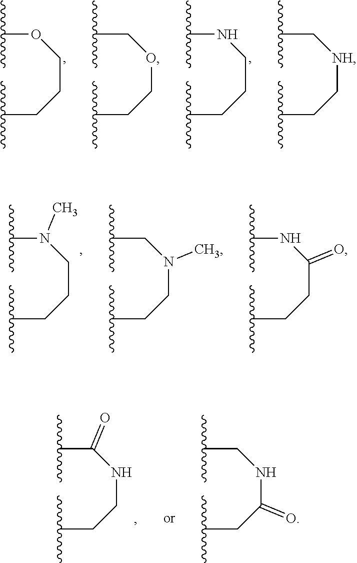

In some cases [symbol] is

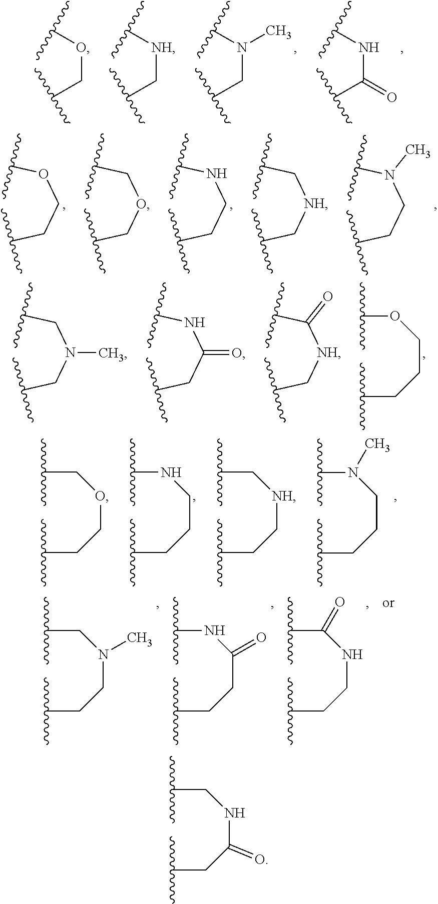

In some cases, is

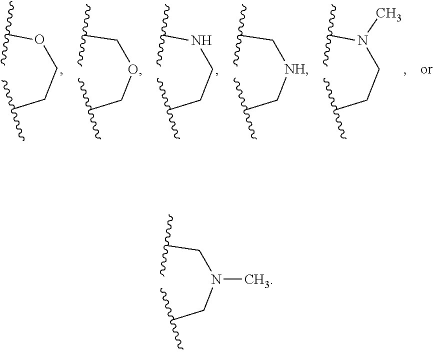

In some cases, [symbol] is heteroalkenylene having 3-6 total atoms

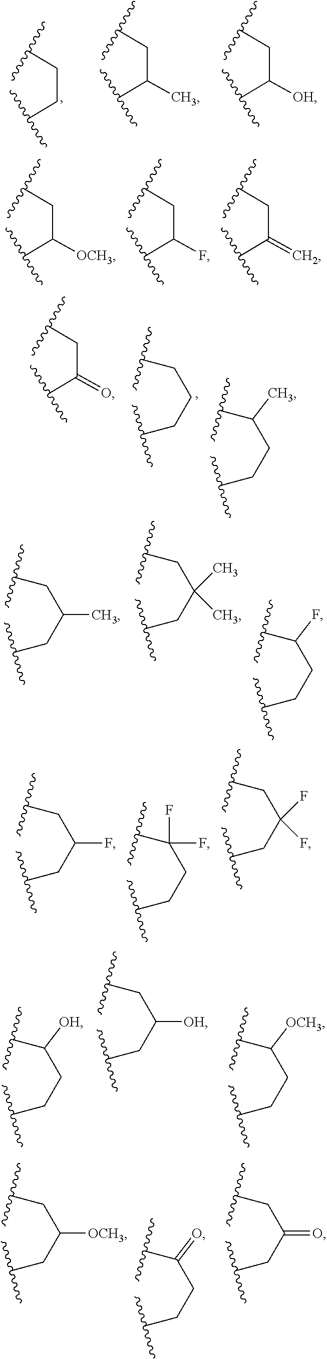

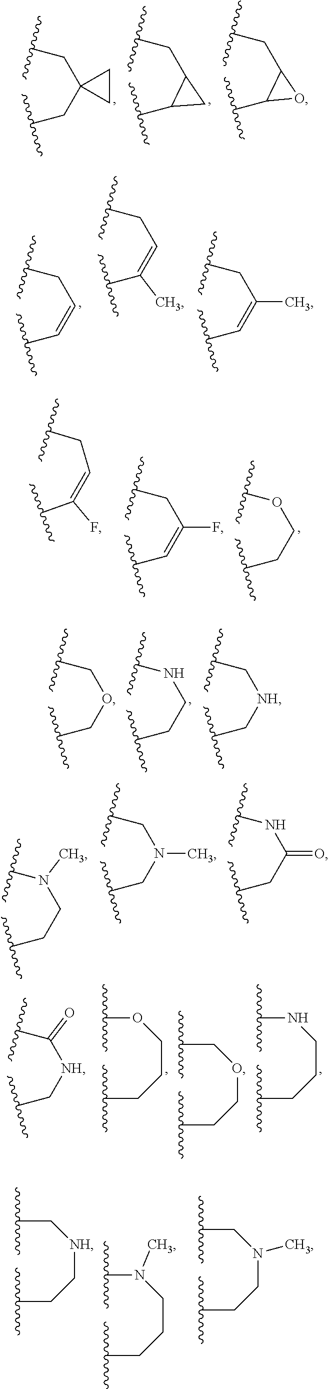

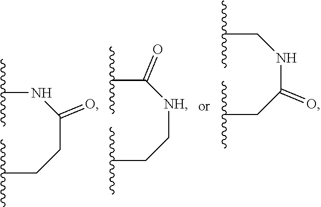

and 1-3 heteroatoms selected from N, O, and S. In some cases, is

In some cases, [symbol] is

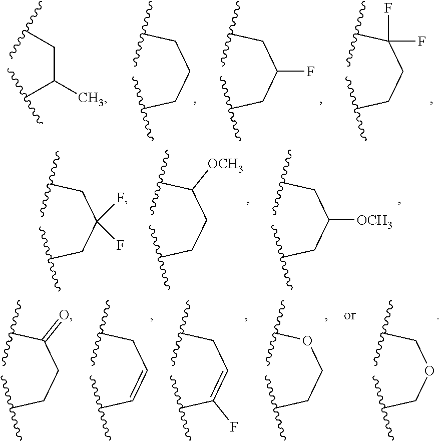

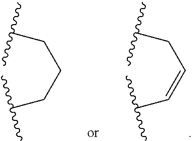 [SMILES: *CCC* ; */C=C\C*]

In some cases,

In some cases, [symbol] is a tether having 2-6 total atoms and 0, 1, or 2 heteroatoms selected from N, O, and S, to form a ring on Formula (II) having 6-10 total atoms, wherein the tether is unsubstituted or substituted. In some cases, [symbol] is saturated. In some cases, is unsaturated. In some cases, [symbol] has 2 total atoms and forms a ring having 6 total ring atoms. In some cases, has 3 total atoms and forms a ring having 7 total ring atoms. In some cases, [symbol] has 4 total atoms and forms a ring having 8 total ring atoms. In some cases, ‘has 5 total atoms and forms a ring having 9 total ring atoms. In some cases, has 6 total atoms and forms a ring having 10 total ring atoms. In some cases,’ has 0 heteroatoms. In some cases, has 1 or 2 heteroatoms selected from N, O, and S. In some cases, has 1 or 2 oxygen atoms. In some cases, is an ether. In some cases, is a polyether. In some cases, has 1 or 2 nitrogen atoms. In some cases, forms a cyclic amide (i.e., lactam). In some cases, forms a cyclic amine. In some cases, is unsubstituted. In some cases, is substituted with 1 or 2 substituents, and each substituent independently is C₁₋₃alkyl, C₁₋₃haloalkyl, halo, CN, C₀₋₃alkyleneOH, C₀₋₃alkylene-C₁₋₃alkoxy, C₃₋₇cycloalkyl, C₅₋₇cycloalkenyl, heterocycloalkyl having 4-7 total ring atoms and 1-3 heteroatoms selected from N, O, and S, heterocycloalkenyl having 5-7 total ring atoms and 1-3 heteroatoms selected from N, O, and S, phenyl, or two geminal substituents, together with the atom to which they are attached, form oxo. In some cases, [symbol] is

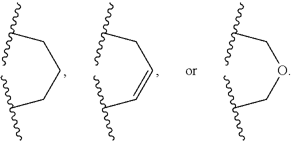 [SMILES: *CCC* ; */C=C\C* ; *COC*]

In some cases, WI is N. In some cases, W¹ is CH. In some cases, WI is C-halo or C—CN. In some cases, W¹ is C—F, C—Cl, or C—Br. In some cases, W¹ is C—F, C—Cl, or C—CN. In some cases, W¹ is C—C₁₋₃alkyl or C—C₁₋₃haloalkyl. In some cases, W¹ is C—CH₃, C—CH₂CH₃, C₂CH₂CH—CH₃, C—CH(CH₃)₂, C—CF₃, C—CHF₂, or C—CH₂F. In some cases, W¹ is C—CH₃, C—CH₂CH₃, C—CH₂F, C—CHF₂, or C—CF₃. In some cases, WI is C—CH₃ or C—CH₂CH₃. In some cases, WI is C—C₂₋₆alkenyl or C—C₂₋₃alkynyl, and each of the alkenyl and alkynyl is unsubstituted or substituted with 1 or more substituents. In some cases, each of the alkenyl and alkynyl is unsubstituted. In some cases, each of the alkenyl and alkynyl is substituted with 1-3 substituents, and each substituent independently is halo, C₁₋₃haloalkyl, C₀₋₃alkyleneOH, or C₀₋₃alkyleneC₁₋₄alkoxy. In some cases, W¹ is C—CH═CH₂, C—C(OH)═CH₃, C—CH═CH(OH), or C—CCH. In some cases, W¹ is C—C₀₋₂alkyleneOH or C—C₀₋₃alkylene-C₁₋₄alkoxy. In some cases, W¹ is C—OH, C—CH₂OH, C—CH₂CH₂OH, C—OCH₃, C—CH₂OCH₃, or C—CH₂CH₂OCH,. In some cases, WI is C—OH, C—CH₂OH, C—OCH₃, or C—CH₂OCH₃. In some cases, WI is CH, C—F, C—Cl, C—CN, C—CH₃, C—CH₂CH₃, C—CH—CH₂CH₃, C—CH(CH₃)₂, C—CF₃, C—CHF₂, C—CH₂F, C—OH, C—CH₂OH, C—CH₂CH₂OH, C—OCH₃, C—CH₂OCH₃, or C₂CH₂CH₂OCH₃. In some cases, W¹ is CH, C—F, C—Cl, C—CN, C—CH₃, C—CH—CH₃, C—OH, C—CH₂OH, C—OCH₃, or C—CH₂OCH₃. In some cases, W¹ is C—F, C—Cl, C—CN, C—CH₃, C—CH₂CH₃, C—CH₂F, C—CHF₂, C—CF₃, C—CH═CH₂, C—C(OH)═CH₂, C—CH═CH(OH), C—CCH, C—OH, C₂CH₂OH, C—OCH₃, or C—CH₂OCH₃. In some cases, W² is N. In some cases. W² is CH. In some cases, W² is C-halo or C—CN. In some cases, W² is C—F, C—Cl, or C—Br. In some cases, W² is C—F, C—Cl, or C—CN. In some cases, W² is C—C₁₋₃alkyl or C—C₁₋₃haloalkyl. In some cases, W² is C—CH₃, C—CHCH₃, C—CH₂CH₂CH₃, C—CH(CH₃)₂, C—CF₃, C—CHF₂, or C—CH₂F. In some cases, W² is C—CH₃, C—CH₂CH₃, C—CH₂F, C—CHF₂, or C—CF₃. In some cases, W² is C—CH₃ or C₂CH₂CH₃. In some cases, W² is C—C₂₋₃alkenyl or C—C₂₋₃alkynyl, and each of the alkenyl and alkynyl is unsubstituted or substituted with 1 or more substituents. In some cases, each of the alkenyl and alkynyl is unsubstituted. In some cases, each of the alkenyl and alkynyl is substituted with 1-3 substituents, and each substituent independently is halo, C₁₋₃haloalkyl, C₀₋₃alkyleneOH, or C₀₋₃alkyleneC₁₋₄alkoxy. In some cases, W² is C—CH═CH₂, C—C(OH)═CH₂, C—CH═CH(OH), or C—CCH. In some cases, W² is C—C₀₋₂alkyleneOH or C—C₀₋₃alkylene-C₁₋₄alkoxy. In some cases, W² is C—OH, C—CH₂OH, C—CH₂CH₂OH, C—OCH₃, C—CH₂OCH₃, or C—CH₂CH₂OCH₃. In some cases, W² is C—OH, C—CH₂OH, C—OCH₃, or C—CH₂OCH₃. In some cases, W² is CH, C—F, C—Cl, C—CN, C—CH₃, C—CH₂CH₃, C—CH₂CH₂CH₃, C—CH(CH₃)₂, C—CF₃, C—CHF₂, C—CH₂F, C—OH, C—CH₂OH, C—CH₂CH₂OH, C—OCH₃, C—CH₂OCH₃, or C—CH₂CH₂OCH₃. In some cases, W^{?} is CH, C—F, C—Cl, C—CN, C—CH₃, C—CH₂CH₃, C—OH, C—CH₂OH, C—OCH₃, or C—CH₂OCH₃. In some cases, W² is C—F, C—Cl, C—CN, C—CH₃, C—CH₂CH₃, C—CH₂F, C—CHF₂, C—CF₃, C—CH═CH₂, C—C(OH)=CH₂, C—CH═CH(OH), C—CCH, C—OH, C₂CH₂OH, C—OCH₃, or C—CH₂OCH₃. In some cases, each of W¹ and W² independently is N, CH, or C₂CH₃. In some cases, W¹ is CH and W² is N, CH, or C₂CH₃. In some cases, W² is N and WI is N, CH, or C—CH₃. In some cases, WI is CH and W² is N. In some cases,

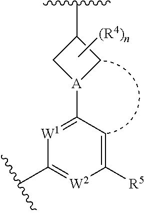

is

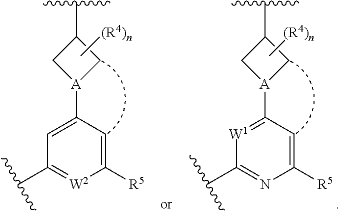

In some cases,

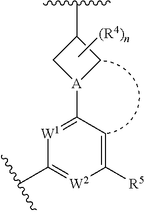

is

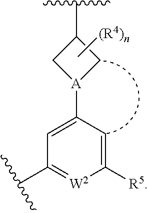

In some cases,

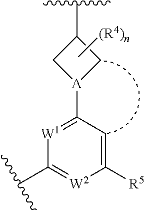

is

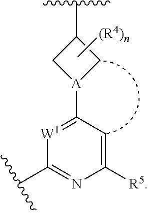

In some cases,

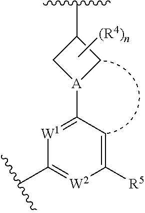

is

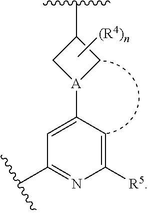

In some cases, R⁵ is halo. In some cases, R⁵ is Br, Cl, or F. In some cases, R⁵ is C₁₋₃haloalkyl. In some cases, R⁵ is CF₃, CF₂H, CFH₂, or CF₂CH₃. In some cases, R⁵ is CF₃ or CF₂H. In some cases, R⁵ is CF₃. In some cases, R⁵ is CF₂H. In some cases, R⁵ is CHF₂. In some cases, R³ is C₁₋₃alkoxy or C₁₋₃thioalkyl. In some cases, R⁵ is OCH₃, OCH₂CH₃, SCH₃, or SCH₂CH₃. In some cases, R⁵ is OCH₃, or SCH₃. In some cases, R⁵ is C₁₋₆alkyl, C₂₋₄alkenyl, or C₂₋₄alkynyl, each of which is unsubstituted or substituted with 1 or more substituents. In some cases, the C₁₋₆alkyl is CH₃, CH₂CH₃, CH₂CH₂CH₃, or CH(CH₃)₂, wherein each of the foregoing is unsubstituted or substituted with 1 or more substituents. In some cases, the C₂₋₄alkenyl is CH═CH₂ or CH═CHCH₃, wherein each of the foregoing is unsubstituted or substituted with 1 or more substituents. In some cases, the C₂₋₄alkynyl is

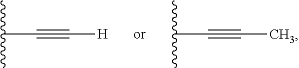

wherein each of the foregoing is unsubstituted or substituted with 1 or more substituents. In some cases, the C₁₋₆alkyl, C₂₋₄alkenyl, and C₂₋₄alkynyl is unsubstituted. In some cases, R⁵ is CH, CH₂CH₃, CH₂CH₂CH, or CH(CH₃)₂. In some cases, the C₁₋₆alkyl, C₂₋₄alkenyl, and C₂₋₄alkynyl is substituted with 1-3 substituents. In some cases, each of the 1-3 substituents independently is C₁₋₆haloalkyl, C₀₋₆alkylene (OH), C₀₋₆alkylene-C₁₋₃alkoxy, C₃₋₇cycloalkyl, C₅₋₇cycloalkenyl, heterocycloalkyl having 3-7 total ring atoms and 1-3 heteroatoms selected from N, O, and S, heterocycloalkenyl having 3-7 total ring atoms and 1-3 heteroatoms selected from N, O, and S. or phenyl. In some cases, each of the 1-3 substituents independently is CH₃, CF₃, CF₂H, CFH₂, OH, OCH₃, OCF₃, CH₂OH, CH₂OCH₃, cyclopropyl, cyclobutyl, or phenyl. In some cases, R⁵ is CH₃, CH₂CH₃, CH₂CH₂CH₃, CH(CH₃)₂,

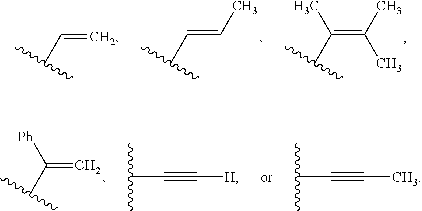

In some cases, R⁵ is C₃₋₇cycloalkyl, C₅₋₇cycloalkenyl, heterocycloalkyl having 3-7 total ring atoms and 1-3 heteroatoms selected from N, O, and S, or heterocycloalkenyl having 5-7 total ring atoms and 1-3 heteroatoms selected from N, O, and S, wherein each of the foregoing is unsubstituted or substituted with 1-3 substituents independently selected from halo, C₁₋₃alkyl, C₁₋₃haloalkyl, C₀₋₃alkylene(OH), or C₀₋₆alkylene-C₁₋₃alkoxy. In some cases, the C₃₋₇cycloalkyl is cyclopropyl, cyclobutyl, cyclopentyl, or cyclohexyl, wherein each of the foregoing is unsubstituted or substituted with 1-3 substituents. In some cases, the C₅₋₇cycloalkenyl is cyclopentenyl or cyclohexenyl, wherein each of the foregoing is unsubstituted or substituted with 1-3 substituents. In some cases, the heterocycloalkyl having 3-7 total ring atoms and 1-3 heteroatoms selected from N, O, and S is aziridinyl, oxiranyl, thiiranyl, azetidinyl, oxetanyl, thietanyl, pyrrolidinyl, imidazolidinyl, pyrazolidinyl, tetrahydrofuranyl, tetrahydrothiopheneyl, oxazolidinyl, oxathiolidinyl, isoxazolidinyl, thiazolidinyl, isothiazolidinyl, piperidinyl, piperazinyl, tetrahydropyranyl, dioxanyl, tetrahydrothipyranyl, dithianyl, morpholinyl, or thiomorpholinyl, wherein each of the foregoing is unsubstituted or substituted with 1-3 substituents. In some cases, the heterocycloalkenyl having 5-7 total ring atoms and 1-3 heteroatoms selected from N, O, and S is dihydropyrrolyl, dihydrofuranyl, dihydrothiopheneyl, dihydroisoxazolyl, tetrahydropyridinyl, dihydropyranyl, or dihydrothipyranyl, wherein each of the foregoing is unsubstituted or substituted with 1-3 substituents. In some cases, R⁵ is cyclopropyl, cyclobutyl, cyclopentenyl, oxetanyl, or tetrahydrofuranyl. In some cases. R⁵ is CH₃, CF₃, CF₂H, CFH₂, CH₂CH₃, CH₂CH₂CH₃, CH(CH₃)₂,

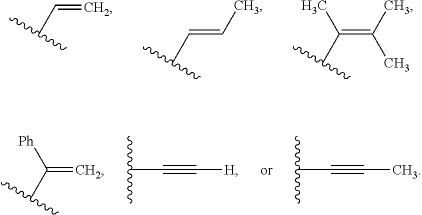

In some cases, R⁵ is Br, Cl, F, OCH₃, SCH₃, CH₃, CH₂CH₃, CH₂CH₂CH₃, CH(CH₃)₂,

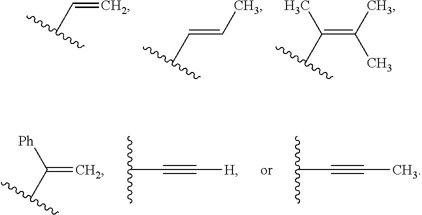

In some cases, W¹ is CH, W² is N, and R⁵ is CF₃, CF₂H, or CFH₂. In some cases,

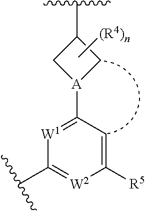

is

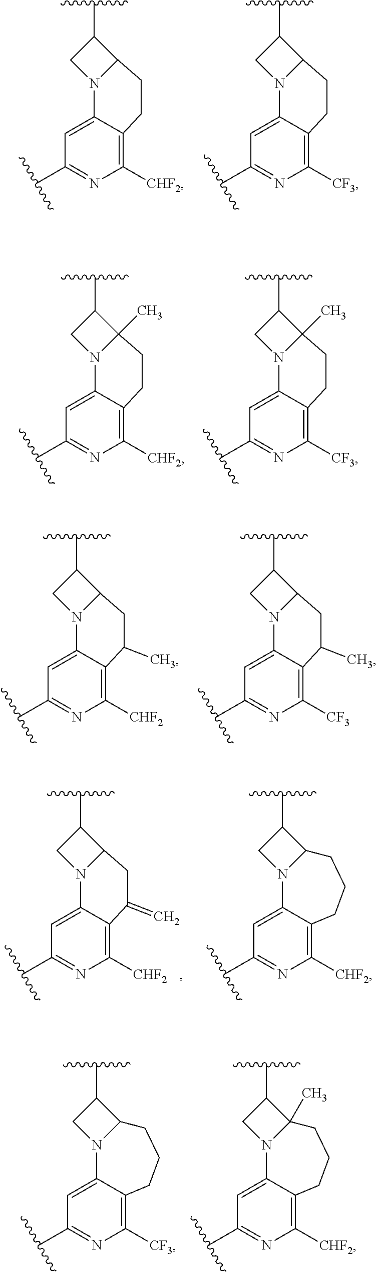

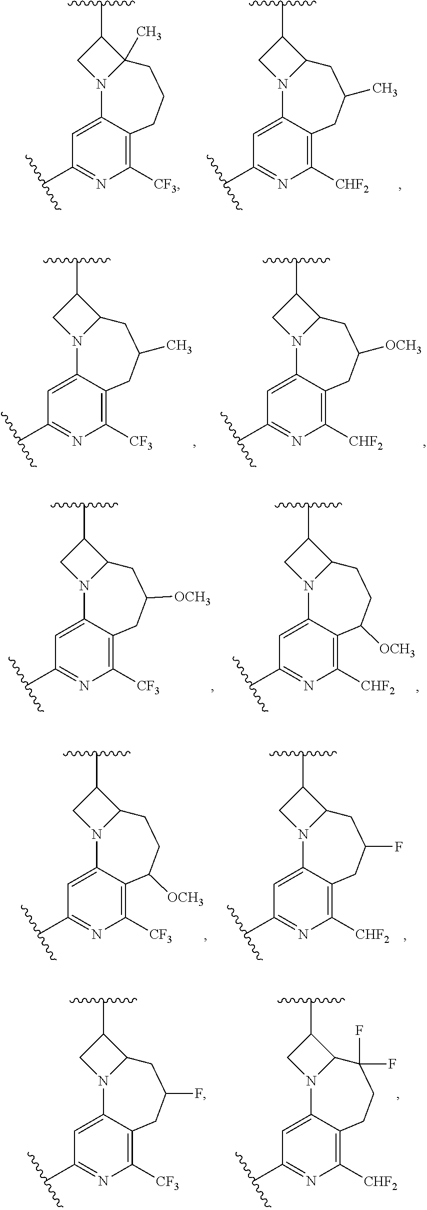

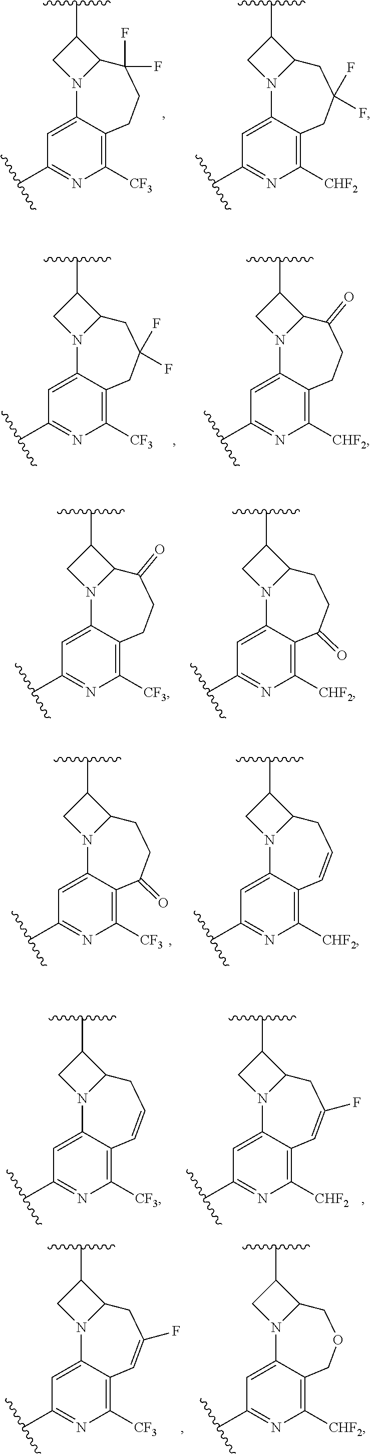

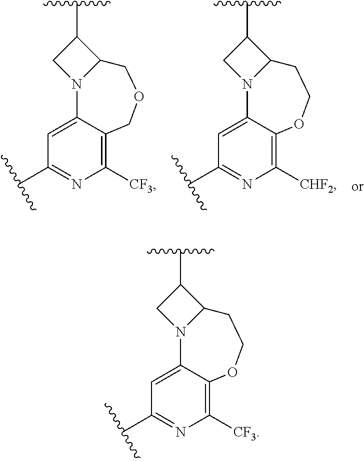 [SMILES: *c1cc2c(c(C(F)(F)F)n1)COCC1C(*)CN21 ; *c1cc2c(c(C(F)F)n1)OCCC1C(*)CN21 ; *c1cc2c(c(C(F)(F)F)n1)OCCC1C(*)CN21]

In some cases,

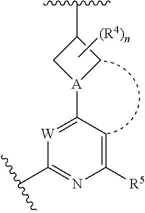

is

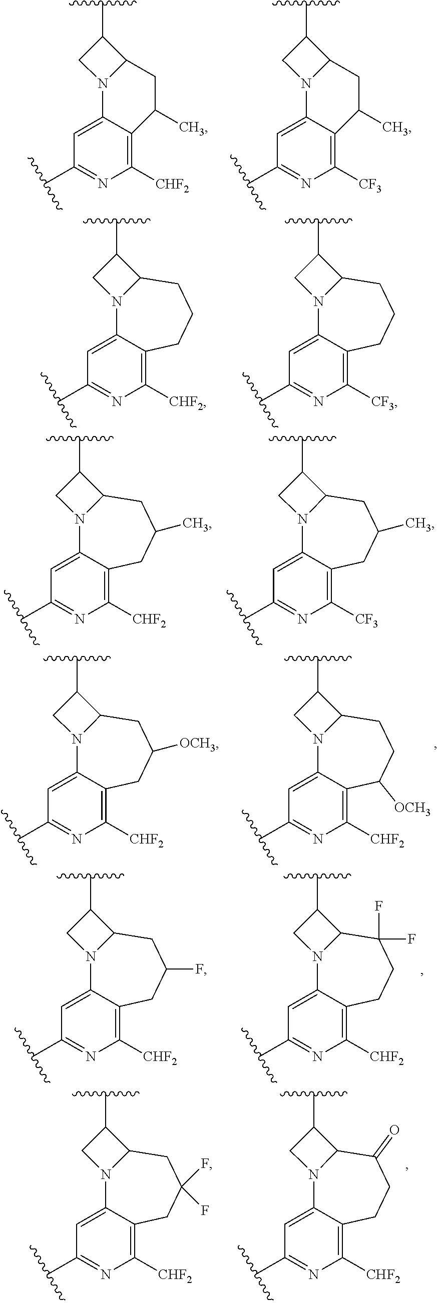

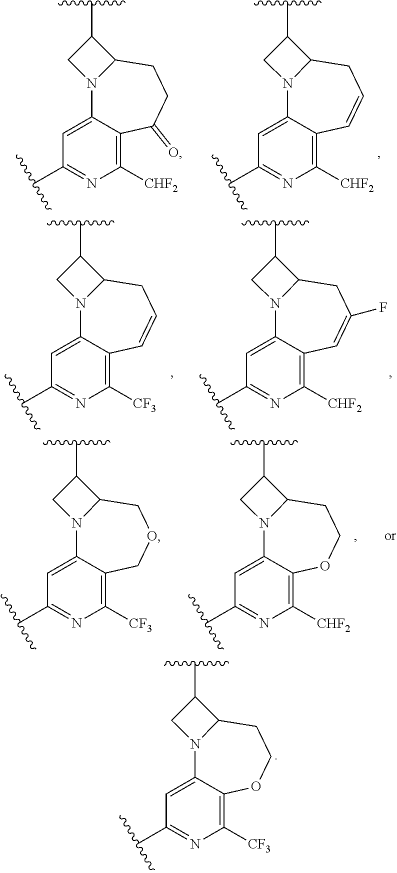

In some cases,

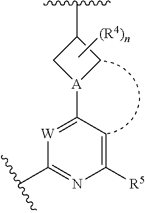

is

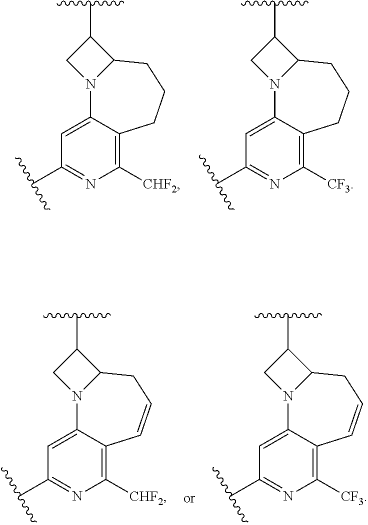 [SMILES: *c1cc2c(c(C(F)F)n1)CCCC1C(*)CN21 ; *c1cc2c(c(C(F)(F)F)n1)CCCC1C(*)CN21 ; *c1cc2c(c(C(F)F)n1)C=CCC1C(*)CN21 ; *c1cc2c(c(C(F)(F)F)n1)C=CCC1C(*)CN21]

In some cases, X is

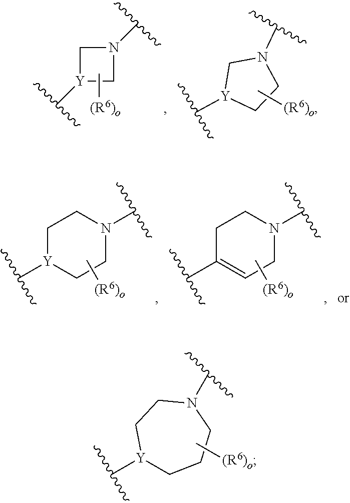

Y is N, C—H, C-halo, C—CN, C—C₁₋₃alkyl, C—C₁₋₃haloalkyl, C—C₀₋₃alkyleneOH, or C—C₀₋₃alkylene-C₁₋₃alkoxy; o is 0, 1, 2, 3, or 4; and each R⁶ independently is halo, CN, C₁₋₃alkyl, C₂₋₃alkenyl, C₁₋₃haloalkyl, C₀₋₃alkylene-OH, C₀₋₃alkylene-C₁₋₃alkoxy, deuterated C₀₋₃alkylene-C₁₋₃alkoxy, or C₁₋₄alkylene-N(R^{N1})₂; two geminal R⁶, together with the atom to which they are attached, form oxo, ═CH₂, spiro-C₃₋₇cycloalkyl, spiro-C₄₋₇cycloalkenyl, spiro-heterocycloalkyl having 4-7 total ring atoms and 1 or 2 heteroatoms selected from N, O and S, or spiro-heterocycloalkenyl having 4-7 total ring atoms and 1 or 2 heteroatoms selected from N, O and S; or two vicinal R⁶, together with the atoms to which they are attached, form fused-C₃₋₇cycloalkyl, fused-C₄₋₇cycloalkenyl, fused-heterocycloalkyl having 4-7 total ring atoms and 1-3 heteroatoms selected from N, O, and S, or fused-heterocycloalkenyl having 4-7 total ring atoms and 1 or 2 heteroatoms selected from N, O, and S; two non-neighboring R⁶ join together to form a C₁₋₃alkylene bridge, a C₂₋₆alkenylene bridge, a C₁₋₃ether bridge, or a C₁₋₃thioether bridge; or Y and a vicinal R⁶, together with the atoms to which they are attached, form fused-C₃₋₇cycloalkyl, fused-C₃₋₇cycloalkenyl, fused-heterocycloalkyl having 3-7 total ring atoms and 1-3 heteroatoms selected from N, O, and S, or fused-heterocycloalkenyl having 4-7 total ring atoms and 1 or 2 heteroatoms selected from N, O, and S; wherein the cycloalkyl, cycloalkenyl, heterocycloalkyl or heterocycloalkenyl of any of the foregoing is unsubstituted or substituted with 1 or more substituents; and each R^{N1} independently is H or C₁₋₄alkyl. In some cases, o is 0. In some cases, o is 1. In some cases, o is 2. In some cases, o is 3. In some cases, o is 4. In some cases, at least one R⁶ is halo or CN. In some cases, at least one R⁶ is Br, Cl, F, or CN. In some cases, at least one R⁶ is F. In some cases, o is 1 or 2 and each R⁶ independently is F. In some cases, at least one R⁶ is C₁₋₆alkyl or C₁₋₃haloalkyl. In some cases, at least one R⁶ is CH₃, CH₂F, CHF₂, or CF₃. In some cases, o is 1 or 2 and each R⁶ independently is CH₃. In some cases, at least one R⁶ is C₀₋₃alkyleneOH, C₀₋₆alkylene-C₁₋₃alkoxy, deuterated C₀₋₃alkylene-C₁₋₄alkoxy, or C₁₋₄alkylene-N(R^{N1})₂, and each R^{N1} independently is H or CH₃. In some cases, each R^{N1} independently is H. In some cases, at least one R⁶ is OH, CH₂OH, CH₂CH₂OH, OCH₃, OCD₃, CH₂OCH₃, or CH₂CH₂OCH₃. In some cases, at least one R⁶ is CH₂N(CH₃)₂, CH₂NH (CH₃), or CH₂NH₂. In some cases, at least one R⁶ is OH, CH₂OH, OCH₃, OCD₃, CH₂OCH₃, or CH₂N(CH₃)₂. In some cases, o is 1 and R⁶ is OH, CH₂OH, OCH₃, or CH₂OCH₃. In some cases, two geminal R⁶ form oxo (═O) or —CH₂. In some cases, two geminal R⁶, together with the atom to which they are attached, form spiro-C₁₋₃-cycloalkyl, spiro-C₄₋₇cycloalkenyl, spiro-heterocycloalkyl having 4-7 total ring atoms and 1 or 2 heteroatoms selected from N, O and S, or spiro-heterocycloalkenyl having 4-7 total ring atoms and 1 or 2 heteroatoms selected from N, O and S, wherein the any of the foregoing is unsubstituted or substituted with 1 or more substituents. In some cases, two geminal R⁶, together with the atom to which they are attached, form spiro-C₃₋₇cycloalkyl or spiro-heterocycloalkyl having 4-7 total ring atoms and 1 or 2 heteroatoms selected from N, O and S, wherein any of the foregoing is unsubstituted or substituted with 1 or more substituents. In some cases, two geminal, R⁶ together with the atom to which they are attached, form spiro-cyclopropyl, spiro-cyclobutyl, spiro-oxetanyl, or spiro-tetrahydrofuranyl, wherein any of the foregoing is unsubstituted or substituted with 1 or more substituents. In some cases, two geminal R⁶, together with the atom to which they are attached, form spiro-cyclopropyl that is unsubstituted or substituted with 1 or more substituents. In some cases, two vicinal R⁶, together with the atoms to which they are attached, form fused-C₃₋₇cycloalkyl, fused-C₄ cycloalkenyl, fused-heterocycloalkyl having 4-7 total ring atoms and 1-3 heteroatoms selected from N, O, and S, or fused-heterocycloalkenyl having 4-7 total ring atoms and 1 or 2 heteroatoms selected from N, O, and S, or Y and a vicinal R⁶, together with the atoms to which they are attached, form fused-C₃-cycloalkyl, fused-C₄₋₇cycloalkenyl, fused-heterocycloalkyl having 3-7 total ring atoms and 1-3 heteroatoms selected from N, O, and S, or fused-heterocycloalkenyl having 4-7 total ring atoms and 1 or 2 heteroatoms selected from N, O, and S; wherein any of the foregoing is unsubstituted or substituted with 1 or more substituents. In some cases, two vicinal R⁶, together with the atoms to which they are attached, form fused-C₃₋₇cycloalkyl, or Y and a vicinal R⁶, together with the atoms to which they are attached, form fused-C₃₋₇cycloalkyl, wherein the cycloalkyl of any of the foregoing is unsubstituted or substituted with 1 or more substituents. In some cases, the fused-C₃₋₇cycloalkyl is fused-cyclopropyl, fused-cyclobutyl, or fused-cyclopentyl, wherein any of the foregoing is unsubstituted or substituted with 1 or more substituents. In some cases, the spiro-cycloalkyl, spiro-cycloalkenyl, spiro-heterocycloalkyl, spiro-heterocycloalkenyl, fused-cycloalkyl, fused-cycloalkenyl, fused-heterocycloalkyl, fused-heterocycloalkenyl of any of the foregoing is unsubstituted. In some cases, the spiro-cycloalkyl, spiro-cycloalkenyl, spiro-heterocycloalkyl, spiro-heterocycloalkenyl, fused-cycloalkyl, fused-cycloalkenyl, fused-heterocycloalkyl, fused-heterocycloalkenyl of any of the foregoing is substituted with 1 or more substituents. In some cases, the spiro-cycloalkyl, spiro-cycloalkenyl, spiro-heterocycloalkyl, spiro-heterocycloalkenyl, fused-cycloalkyl, fused-cycloalkenyl, fused-heterocycloalkyl, fused-heterocycloalkenyl of any of the foregoing is unsubstituted. In some cases, the spiro-cycloalkyl, spiro-cycloalkenyl, spiro-heterocycloalkyl, spiro-heterocycloalkenyl, fused-cycloalkyl, fused-cycloalkenyl, fused-heterocycloalkyl, fused-heterocycloalkenyl of any of the foregoing is substituted with 1-4 substituents. In some cases, the spiro-cycloalkyl, spiro-cycloalkenyl, spiro-heterocycloalkyl, spiro-heterocycloalkenyl, fused-cycloalkyl, fused-cycloalkenyl, fused-heterocycloalkyl, fused-heterocycloalkenyl of any of the foregoing is substituted with 1 or 2 substituents. In some cases, each substituent of the spiro-cycloalkyl, spiro-cycloalkenyl, spiro-heterocycloalkyl, spiro-heterocycloalkenyl, fused-cycloalkyl, fused-cycloalkenyl, fused-heterocycloalkyl, and fused-heterocycloalkenyl independently is halo, C₁₋₃alkyl, C₁₋₃haloalkyl, C₀₋₂alkyleneOH, C₀₋₂alkyleneC₁₋₃alkoxy, or C₀₋₂alkyleneCN. In some cases, each substituent independently is halo, OH, C₁₋₃alkoxy, or CN. In some cases, each substituent independently is F, Cl, OH, OCH₃, OCH₂CH₃, or CN. In some cases, two non-neighboring R⁶ join together to form a C₁₋₃alkylene bridge, a C₂₋₃alkenylene bridge, a C₁₋₃ether bridge, or a C₁₋₃thioether bridge. In some cases, two non-neighboring R⁶ join together to form a C₀₋₃alkylene bridge, a C₂₋₆alkenylene bridge, or a C₁₋₃ether bridge. In some cases, two non-neighboring R⁶ join together to form a C₁₋₃alkylene bridge or a C₂₋₆alkenylene bridge. In some cases, two non-neighboring R⁶ join together to form a C₀₋₃alkylene bridge or a C₂₋₃alkenylene bridge. In some cases, two non-neighboring R⁶ join together to form a C₁₋₃ether bridge or a C₁₋₃thioether bridge.

In some cases, two non-neighboring R⁶ join together to form a C₁alkylene bridge

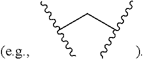 [SMILES: *C*]

In some cases, two non-neighboring R⁶ join together to form a C₁₋₂alkylene bridge

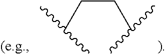 [SMILES: *CC*]

In some cases, two non-neighboring R⁶ join together to form a C₃alkylene bridge

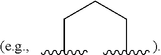 [SMILES: *CCC*]

In some cases, two non-neighboring R⁶ join together to form a C₁₋₃alkenylene bridge

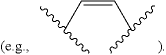 [SMILES: */C=C\*]

In some cases, two non-neighboring R⁶ join together to form a C₃alkenylene bridge

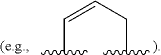 [SMILES: */C=C\C*]

In some cases, two non-neighboring R⁶ join together to form a C₁₋₃ether bridge

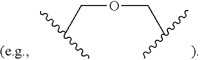 [SMILES: *COC*]

In some cases, two non-neighboring R⁶ join together to form a C₁₋₃thioether bridge

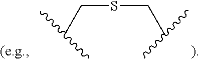 [SMILES: *CSC*]

In some cases, two non-neighboring R⁶ join together to form —CH₂—, —CH₂CH₂—, —CH₂CH₂CH₂—, —CH₂—CH—CH— or —CH₂OCH₂—. In some cases, Y is N. In some cases, Y is CH. In some cases, Y is C-halo, C—CN, C—C₁₋₃alkyl, C—C₁₋₃haloalkyl, C—C₀₋₃alkyleneOH, or C—C₀₋₃alkylene-C₁₋₄alkoxy. In some cases, Y is C—F, C—Cl, or C—CN. In some cases, Y is C—C₁₋₃alkyl or C—C₁₋₃haloalkyl. In some cases, Y is C—CH₃, C—CH₂CH₃, C—CH₂F, C—CHF₂, or C—CF₃. In some cases, Y is C—C₀₋₃alkyleneOH or C—C₀₋₃alkylene-C₁₋₄alkoxy. In some cases, Y is C—OH, C—CH₂OH, C—OCH₃, or C—CH₂OCH₃. In some cases, X is

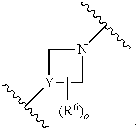

In some cases, X is

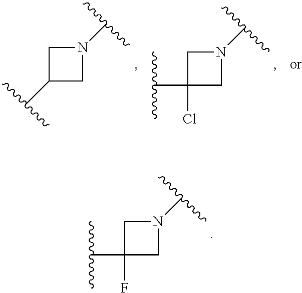 [SMILES: *C1CN(*)C1 ; *N1CC(*)(Cl)C1 ; *N1CC(*)(F)C1]

In some cases, X is

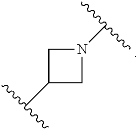 [SMILES: *C1CN(*)C1]

In some cases, X is

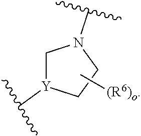

In some cases. X is

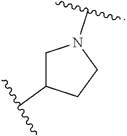 [SMILES: *C1CCN(*)C1]

In some cases, X is

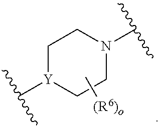

In some cases, X is

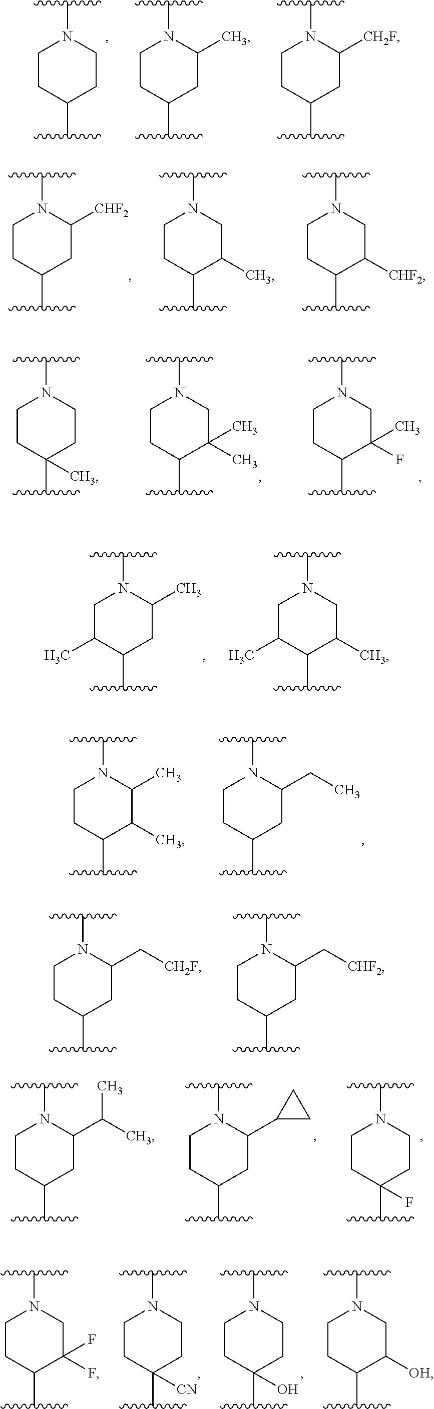

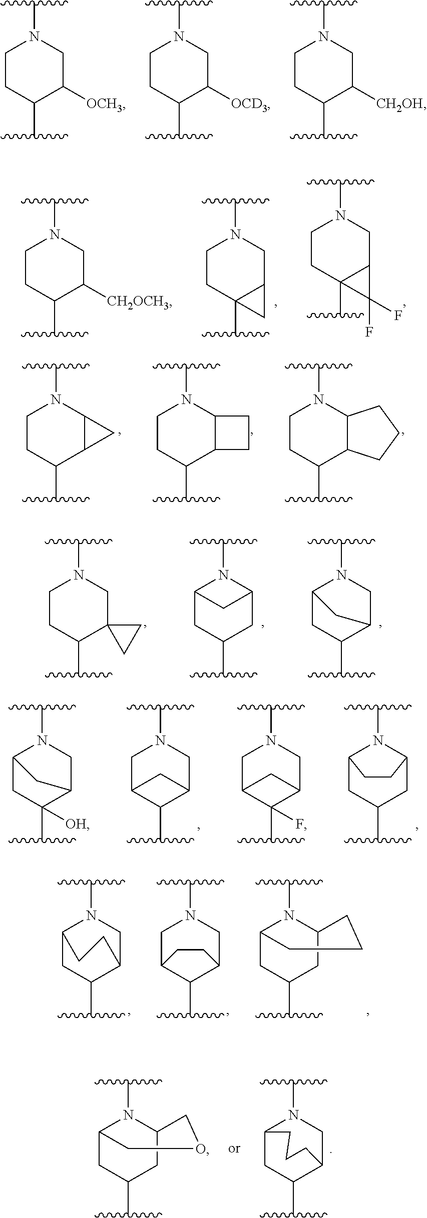

In some cases, X is

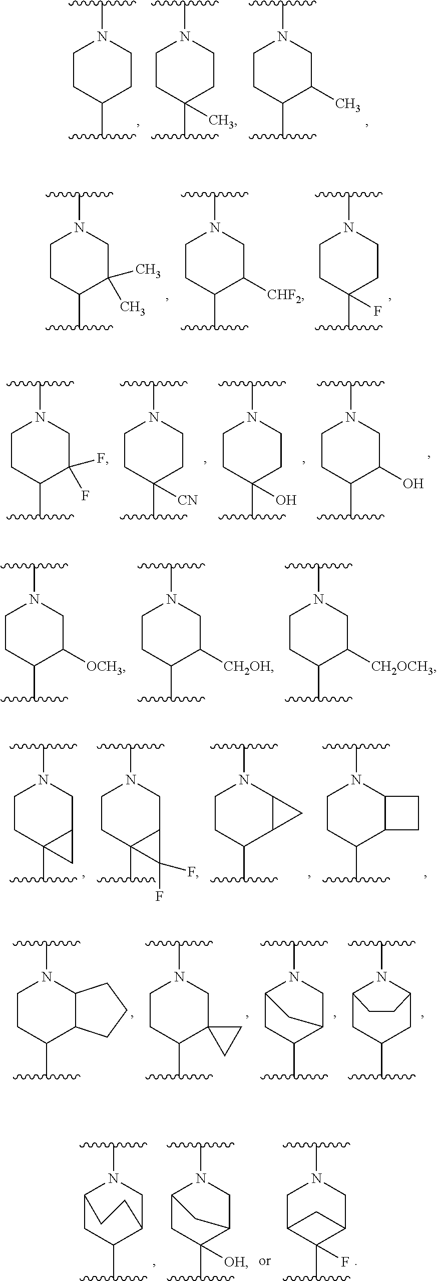

In some cases, X is

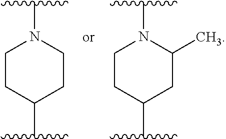 [SMILES: *C1CCN(*)CC1 ; *C1CCN(*)C(C)C1]

In some cases, X is

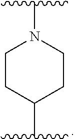 [SMILES: *C1CCN(*)CC1]

In some cases. X is

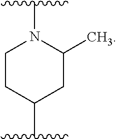 [SMILES: *C1CCN(*)C(C)C1]

In some cases, X is

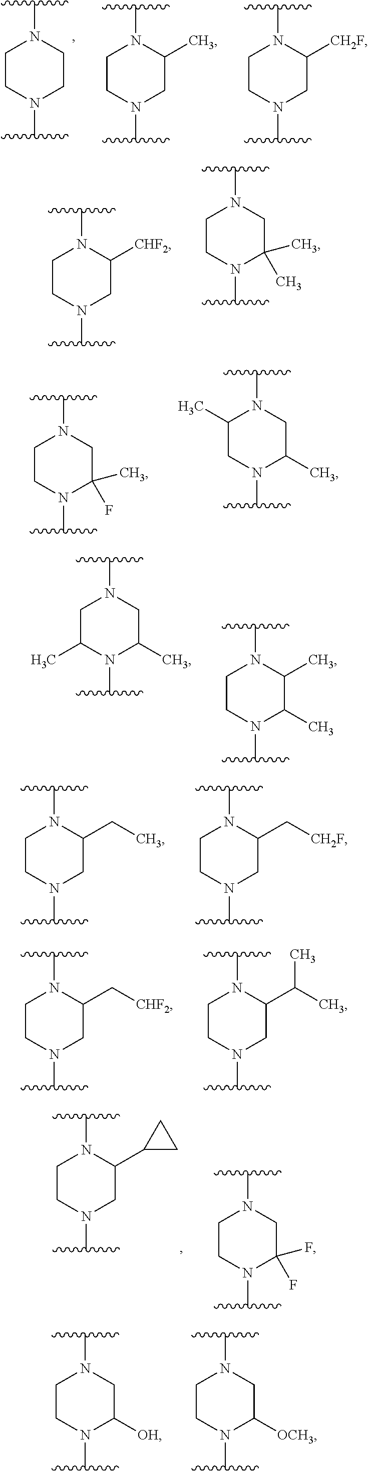

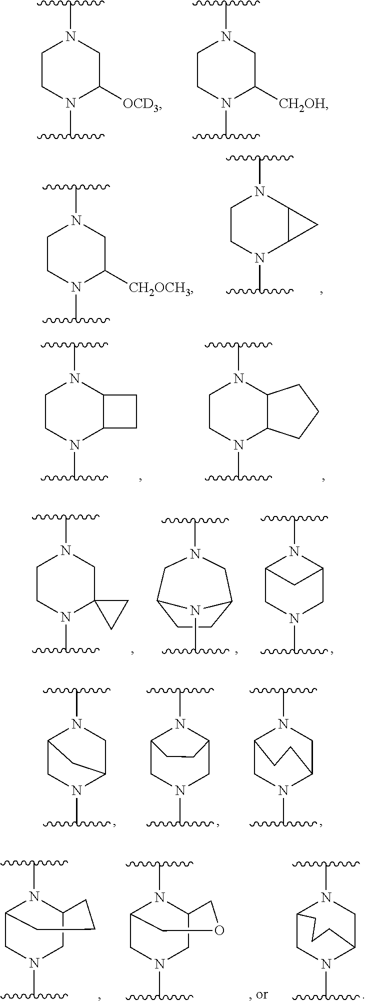

In some cases, X is

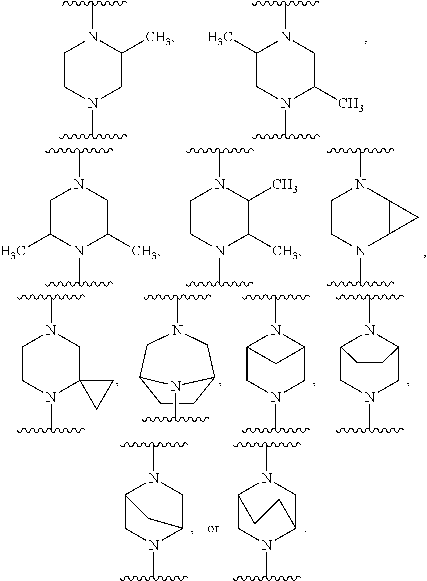

In some cases, X is

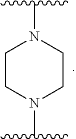 [SMILES: *N1CCN(*)CC1]

In some cases, X is

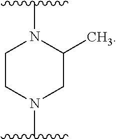 [SMILES: *N1CCN(*)C(C)C1]

In some cases, X is

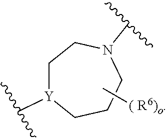

In some cases, X is

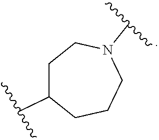 [SMILES: *C1CCCN(*)CC1]

In some cases, X is

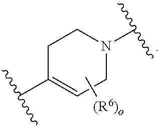

In some cases, X is

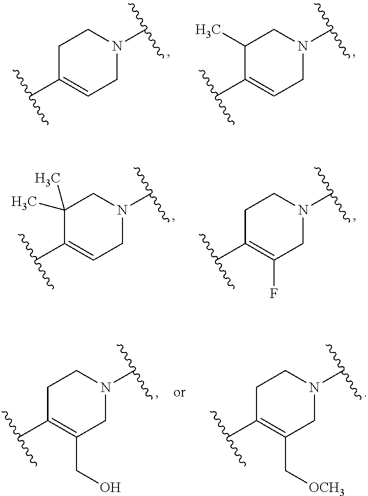

In some cases, X is

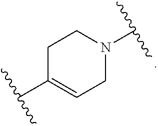 [SMILES: *C1=CCN(*)CC1]

In some cases, X is

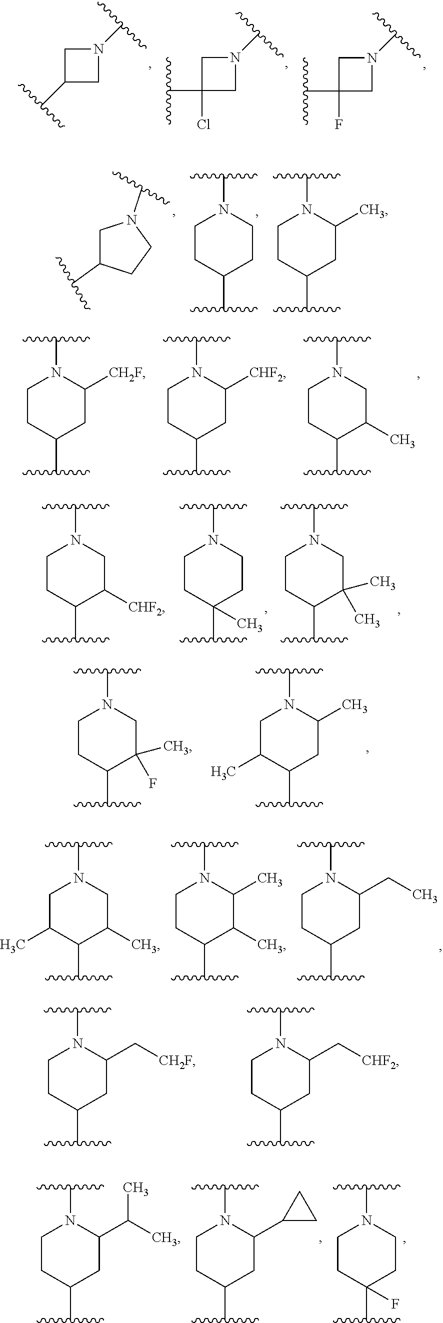

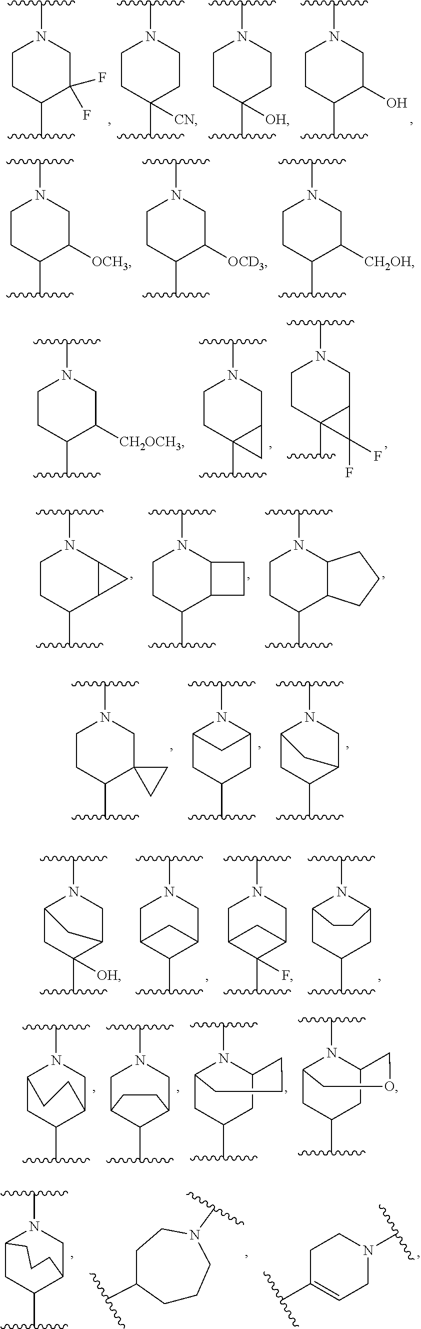

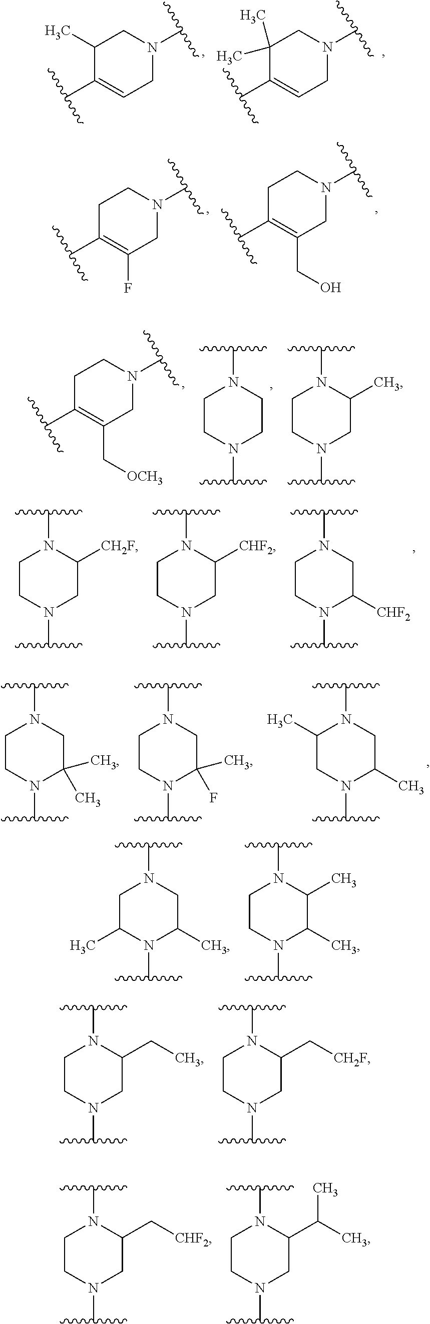

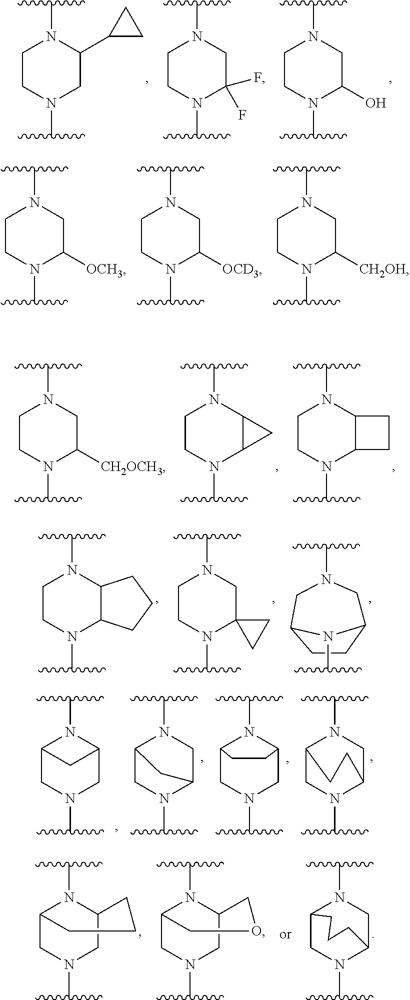

In some cases, X is

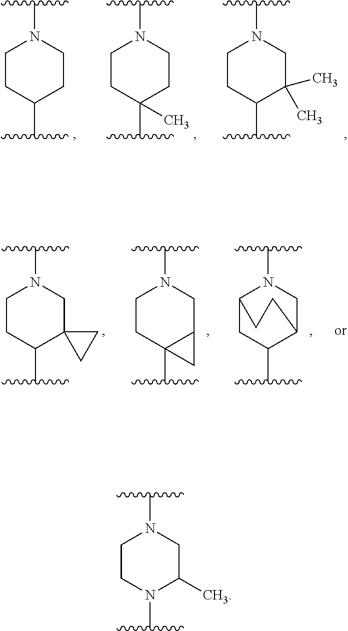

In some cases, X is

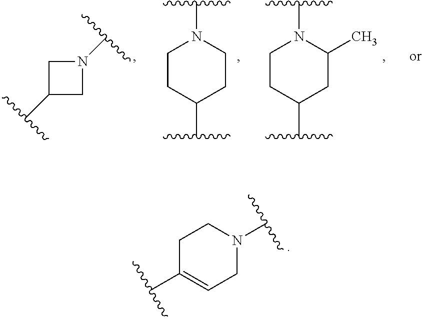

In some cases, Z is unsubstituted phenyl or phenyl substituted with 1 or more substituents. In some cases, Z is unsubstituted phenyl. In some cases, Z is phenyl substituted with 1-4 substituents. In some cases, each of the phenyl substituents independently is halo, C₀₋₃alkyleneCN, C₀₋₃alkyleneOH, C₀₋₆alkylene-C₁₋₄alkoxy, C₀₋₆alkylene-C₁₋₄thioalkyl, or

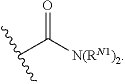

In some cases, each R^{N1} independently H or C₁₋₃alkyl. In some cases, each R^{N1} independently is H or CH₃. In some cases, each R^{N1} is H. In some cases, each of the phenyl substituents independently is F, Cl, CN, OCH₂. SCH₃, CH₂OH, or

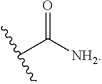 [SMILES: *C(N)=O]

In some cases, Z is

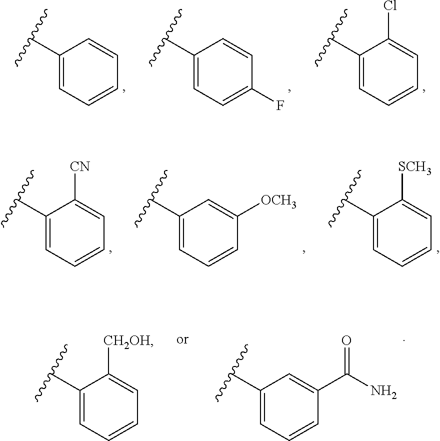

In some cases, Z is

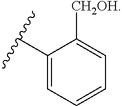 [SMILES: *c1ccccc1CO]

In some cases, Z is heteroaryl comprising 5 or 6 total ring atoms and 1-3 heteroatoms selected from N, O, and S, wherein the heteroaryl is unsubstituted or substituted with 1 or more substituents. In some cases, the heteroaryl comprises 5 total ring atoms. In some cases, the heteroaryl comprises 6 total ring atoms. In some cases, the heteroaryl is pyrrolyl, furanyl, thiophenyl, pyrazolyl, imidazolyl, thiazolyl, isothiazolyl, oxazolyl, isoxazolyl, triazolyl, thiadiazolyl, oxadiazolyl, pyridyl, pyridazinyl, pyrimidinyl, pyrazinyl, or triazinyl. In some cases, the heteroaryl is pyrrolyl, furanyl, thiophenyl, pyrazolyl, imidazolyl, thiazolyl, isothiazolyl, oxazolyl, isoxazolyl, triazolyl, thiadiazolyl, oxadiazolyl, or triazolyl. In some cases, the heteroaryl is pyrazolyl, imidazolyl, thiazolyl, isothiazolyl, oxazolyl, isoxazolyl, or triazolyl. In some cases, the heteroaryl is pyrazolyl, imidazolyl, thiazolyl, or isothiazolyl. In some cases, the heteroaryl is pyrazolyl. In some cases, the heteroaryl is imidazolyl. In some cases, the heteroaryl is thiazolyl. In some cases, the heteroaryl is isothiazolyl. In some cases, the heteroaryl is pyridyl, pyridazinyl, pyrimidinyl, or pyrazinyl. In some cases, the heteroaryl pyridyl. In some cases, the heteroaryl is pyrazolyl, thiazolyl, pyridyl, or pyridazinyl. In some cases, the heteroaryl is pyrazolyl or pyridyl.

In some cases, the heteroaryl is unsubstituted. In some cases, the heteroaryl is substituted with 1-4 substituents. In some cases, the heteroaryl is substituted with 1 or 2 substituents. In some cases, the heteroaryl is substituted with 3 or 4 substituents. In some cases, the heteroaryl is substituted with 1 substituent. In some cases, the heteroaryl is substituted with 2 substituents. In some cases, the heteroaryl is substituted with 3 substituents. In some cases, the heteroaryl is substituted with 4 substituents. In some cases, each of the heteroaryl substituents independently is halo, CN, C₁₋₃alkyl, C₁₋₆haloalkyl, C₂₋₆alkenyl, C₂₋₆haloalkenyl, C₀₋₃alkylene-OH, C₀₋₆alkylene-C₁₋₃alkoxy, C₀₋₆alkylene-N(R^{N1})₂ C₀₋₂alkylene-C₃₋₆cycloalkyl. C₀₋₂alkylene-heterocycloalkyl having 3-6 total ring atoms and 1-3 heteroatoms selected from N, O, and S, or C₀₋₂alkylene-phenyl, wherein each of the C₁₋₃alkyl, C₂₋₆alkenyl. C₀₋₆alkylene-C₁₋₃alkoxy, C₃₋₇cycloalkyl, heterocycloalkyl, and phenyl substituents independently is optionally substituted with 1 or more further substituents, and each R^{N1} independently is H, or C₁₋₃alkyl. In some cases, the C₁₋₆alkyl, C₂₋₆alkenyl, C₀₋₆alkylene-C₁₋₃alkoxy, C₃₋₇cycloalkyl, heterocycloalkyl, and phenyl substituents of the heteroaryl is not further substituted. In some cases, the C₁₋₆alkyl, C₂₋₃alkenyl, C₀₋₃alkylene-C₁₋₃alkoxy, C₃₋₇cycloalkyl, heterocycloalkyl, and phenyl substituents of the heteroaryl is substituted with 1 or more further substituents. In some cases, the C₁₋₆alkyl, C₂₋₆alkenyl, C₀₋₆alkylene-C₁₋₃alkoxy, C₃₋₇cycloalkyl, heterocycloalkyl, and phenyl substituents of the heteroaryl is substituted with 1-3 further substituents. In some cases, the C₁₋₃alkyl, C₂₋₆alkenyl, C₀₋₃alkylene-C₁₋₆alkoxy, C₃₋₇cycloalkyl, heterocycloalkyl, and phenyl substituents of the heteroaryl is substituted with 1 or 2 further substituents. In some cases, the C₁₋₆alkyl, C₂₋₆alkenyl, C₀₋₃alkylene-C₁₋₃alkoxy, C₃₋₇cycloalkyl, heterocycloalkyl, and phenyl substituents of the heteroaryl is substituted with 1 further substituent.

In some cases, each further substituent independently is D, halo, OH, C₁₋₃alkyl, C₁₋₃haloalkyl, C₁₋₂alkyleneOH, C₁₋₂alkylene-C₁₋₃alkoxy, C₁₋₃deuterated alkoxy, N(R^{N1})₂, (C═O)C₁₋₃alkyl, C₃₋₅cycloalkyl, or heterocycloalkyl having 3-5 total ring atoms and 1 or 2 heteroatoms selected from N, O, and S; or two geminal further substituents, together with the atom to which they are attached, form spiro-C₃₋₅cycloalkyl or spiro-heterocycloalkyl having 3-5 total ring atoms and 1 or 2 heteroatoms selected from N, O, and S; or two vicinal further substituents, together with the atoms to which they are attached, form fused-C₃₋₅cycloalkyl or fused-heterocycloalkyl having 3-5 total ring atoms and 1 or 2 heteroatoms selected from N, O, and S; wherein each of the foregoing cycloalkyl and heterocycloalkyl groups independently is unsubstituted or substituted with halo, C₁₋₃alkyl, or a combination thereof, and each R^{N1} independently is H or C₁₋₃alkyl. In some cases, each further substituent independently is D, Br, Cl, F, OH, CH₃, OCH₃, OCD₃, N(CH₃)₂, (C═O) C₁₋₃alkyl, oxetanyl, or azetidinyl; or two geminal further substituents, together with the atom to which they are attached, form spiro-oxetanyl or spiro-azetidinyl, wherein each of the foregoing oxetanyl, azetidinyl, spiro-oxetanyl, and spiro-azetidinyl independently is unsubstituted or substituted with F, CH₃, or a combination thereof. In some cases, each further substituent independently is D, Br, Cl, F, OH, CH₃, CF₃, CF₂H, CFH₂, OCH₃, OCD₃, CH₂OCH₃, N(CH₃)₂, (C═O)CH₃, oxetanyl, or azetidinyl; wherein each of the foregoing oxetanyl, or azetidinyl, or two geminal further substituents, together with the atom to which they are attached, form spiro-oxetanyl, or spiro-azetidinyl; wherein each of the foregoing oxetanyl, azetidinyl, spiro-oxetanyl, and spiro-azetidinyl is unsubstituted or substituted with F, CH₃, or a combination thereof. In some cases, each further substituent independently is D, Br, Cl, F, OH, CH₃, CF₃, CF₂H, CFH₂, OCH₃, OCD₃, N(CH₃), (C═O)CH₃,

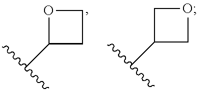 [SMILES: *C1CCO1 ; *C1COC1]

or two geminal further substituents, together with the atom to which they are attached, form

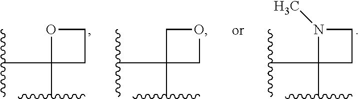

In some cases, each further substituent independently is D, CH₃, OCH₃, OCD₃, N(CH₃)₂,

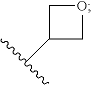 [SMILES: *C1COC1]

or two geminal further substituents, together with the atom to which they are attached, form

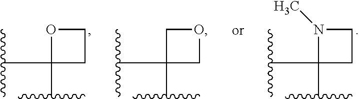

In some cases, the heteroaryl is substituted with Br, Cl, F, or a combination thereof. In some cases, the heteroaryl is substituted with F. In some cases, the heteroaryl is substituted with CN. In some cases, the heteroaryl is substituted with C₁₋₆alkyl, wherein the alkyl is optionally substituted with 1 or more further substituents. In some cases, the heteroaryl is substituted with CH₃, CH₂CH₃, CH₂CH₂CH₃, or CH(CH₃)₂, wherein each of the foregoing independently is optionally substituted with 1 or more further substituents. In some cases, heteroaryl is substituted with CH₃ that is optionally substituted with 1 or more further substituents. In some cases, the C₁₋₆alkyl is unsubstituted. In some cases, the C₁₋₃alkyl is CH₃, CH₂CH₃, CH₂CH₂CH₃, or CH(CH₃)₂. In some cases, the heteroaryl is substituted with C₁₋₃alkyl. In some cases, the C₁₋₆alkyl is substituted with 1-3 substituents, and each of the 1-3 substituents independently is deuterium and halo. In some cases, the substituted C₁₋₆alkyl is CD₃. In some cases, the heteroaryl is substituted with C₁₋₆haloalkyl. In some cases, the C₁₋₆haloalkyl is CF₃, CHF₂, CH₂F, CH₂CHF₂, CH₂CH₂F, CH(CH₂F)₂, CH(CH₃)CH₂F, or CH(CH₃)CHF₂. In some cases, the heteroaryl is substituted with C₂₋₆alkenyl, wherein the alkenyl is optionally substituted with 1 or more further substituents. In some cases, the C₂₋₆alkenyl is CH═CH₂, CH₂CH═CH₂, or CH═CHCH₃, wherein each of the foregoing independently is optionally substituted with 1 or more further substituents. In some cases, the C₂₋₆alkenyl is unsubstituted. In some cases, the C₂₋₆alkenyl is CH═CH₂, CH₂CH═CH₂, or CH═CHCH₃. In some cases, the C₂₋₆alkenyl is substituted with 1-3 substituents, and each of the 1-3 substituents independently is deuterium, halo, OH, OCH₃, and OCD₃. In some cases, the heteroaryl is substituted with C₂₋₆ haloalkenyl. In some cases, the C₂₋₆haloalkenyl is C(═CH₂)CH₂F. In some cases, the heteroaryl is substituted with C₀₋₆alkylene-OH. In some cases, the C₀₋₆alkylene-OH is OH, CH₂OH, CH₂CH₂OH, CH(CH₃)CH₂OH, C(CH₃)₂OH, C(CH₃)₂CH₂OH, or CH₂C(CH₃)₂OH. In some cases, the C₀₋₆alkylene-OH is OH, CH₂OH, CH₂CH₂OH, or C(CH₃)₂CH₂OH. In some cases, the heteroaryl is substituted with C₀₋₆alkylene-C₁₋₃alkoxy, wherein the alkoxy is optionally substituted with 1 or more further substituents. In some cases, the C₀₋₆alkylene-C₁₋₃alkoxy is OCH₃, CH₂OCH₃, CH₂CH₂OCH₃, CH₂CH₂OCH₂CH₃, CH₂CH CH₂OCH₃, CH(CH₃)OCH₃, CH(CH)CH₂OCH₃, CH(OCH₃)CH₂OCH₃, CH(CH₃)(OCH₃)CH₂OCH₃, C(CH₃)₂OCH₃, C(CH₃)₂CH₂OCH₃, CH₂CH(CH₃)OCH₃, CH₂(CH₃)(OCH₃)OCH₃, CH₂C(CH₃)₂OCH₃, or CH₂C(CH₃)₂OCH₃, wherein each of the foregoing independently is optionally substituted with 1 or more further substituents. In some cases, the C₀₋₆alkylene-C₁₋₃alkoxy is OCH₃, CH₂OCH₃, CH₂CH₂OCH₃, or CH₂CH₂CH₂OCH₃, wherein each of the foregoing independently is optionally substituted with 1 or more further substituents. In some cases, the C₀₋₆alkylene-C₁₋₃alkoxy is CH(CH₃)OCH₃ or CH₂CH₂OCH₃, and each of the foregoing independently is optionally substituted with 1 or more further substituents. In some cases, the heteroaryl is substituted with OCH₃, OCD₃, CH₂OCH₃, CH₂OCD₃, CH₂CH₂OCH₃, CH₂CH₂OCD₃, CHFCH₂OCH₃, CF₂CH₂OCH; CH₂CH₂CH₂OCH₃, CH₂CH₂CH₂OCD₃, CH(CH₃)CH₂OCH₃, C(CH₃)₂CH₂OCH₃, CH₂CH(CH₃)OCH₃, CH₂C(CH₃)₂OCH₃, CH(CH₃)CH₂OCD₃, C(CH₃)₂CH₂OCD₃, CH₂CH(CH₃)OCD₃, CH₂C(CH₃),OCD₃, or a combination of the foregoing. In some cases, the heteroaryl is substituted with C₀₋₃alkylene-N(R^{N1})₂. In some cases, the C₀₋₆alkylene-N(R^{N1})₂ is NH₂, CH₂NH₂, CH₂NHCH₃, CH₂N(CH₃)₂, CH₂CH₂NH₂, CH₂CH₂NHCH₃, or CH₂CH₂N(CH₃)₂. In some cases, the heteroaryl is substituted with C₀₋₂alkylene-C₃₋₆ cycloalkyl, wherein the cycloalkyl is optionally substituted with 1 or more further substituents. In some cases, the cycloalkyl of the C₀₋₂alkylene-C₃₋₆cycloalkyl is cyclopropyl, cyclobutyl, cyclopentyl, or cyclohexyl, wherein each of the foregoing independently is optionally substituted or substituted with 1 or more further substituents. In some cases, the cycloalkyl of the C₀₋₂alkylene-C₃₋₅cycloalkyl is cyclopropyl or cyclobutyl, wherein each of the foregoing independently is optionally substituted with 1 or more further substituents. In some cases, each substituent independently is halo, OH, CH₃, OCH₃, or OCD₃. In some cases, the C₀₋₂alkylene-cycloalkyl is substituted with 1-3 substituents, and each substituent independently is Br, Cl, F, OH, CH₃, OCH₃, or OCD₃. In some cases, the optionally substituted C₀₋₂alkylene-cycloalkyl is

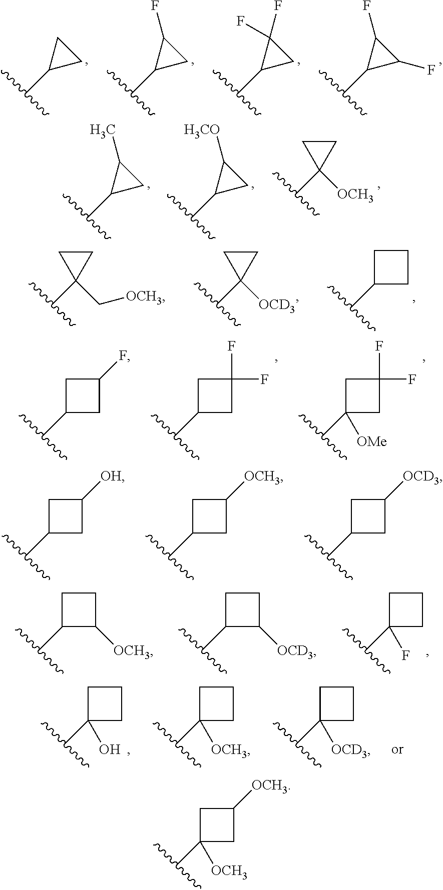

In some cases, the heteroaryl is substituted with C₀₋₂alkylene-heterocycloalkyl having 3-6 total ring atoms and 1-3 heteroatoms selected from N, O, and S. In some cases, the heterocycloalkyl of the C₀₋₂alkylene-heterocycloalkyl is azetidinyl, pyrrolidinyl, piperidinyl, pyrazolidinyl, oxetanyl, tetrahydrofuranyl, tetrahydropyranyl, isoxazolidinyl, or morpholinyl, wherein each of the foregoing independently is optionally substituted with 1 or more further substituents. In some cases, the heterocycloalkyl of the C₀₋₂alkylene-heterocycloalkyl is azetidinyl, oxetanyl, tetrahydrofuranyl, tetrahydropyranyl, or morpholinyl, and each of the foregoing is optionally substituted with 1 or more further substituents. In some cases, the heterocycloalkyl of the optionally substituted C₀₋₂alkylene-heterocycloalkyl is azetidinyl, oxetanyl, pyrrolidinyl, pyrazolidinyl, tetrahydrofuranyl, tetrahydropyranyl, or piperidinyl. In some cases, the heterocycloalkyl of the C₀₋₂alkylene-heterocycloalkyl is azetidinyl or oxetanyl, wherein each of the foregoing independently is optionally substituted with 1 or more further substituents. In some cases, the heterocycloalkyl of the C₀₋₂alkylene-heterocycloalkyl is azetidinyl, wherein the azetidinyl is optionally substituted with 1 or more further substituents. In some cases, the heterocycloalkyl of the C₀₋₂alkylene-heterocycloalkyl is oxetanyl, wherein the oxetanyl is optionally substituted with 1 or more further substituents. In some cases, the C₀₋₂alkylene-heterocycloalkyl is unsubstituted. In some cases, the C₀₋₂alkylene-heterocycloalkyl is substituted with 1-3 further substituents. In some cases, the C₀₋₂alkylene-heterocycloalkyl is substituted with 1 or 2 further substituents. In some cases, the C₀₋₂alkylene-heterocycloalkyl is substituted with 2 further substituents. In some cases, the C₀₋₂alkylene-heterocycloalkyl is substituted with 1 further substituent. In some cases, each further substituent independently is halo, OH, CH₃, OCH₃, or OCD₃. In some cases, each further substituent independently is Br, Cl, F, OH, CH₃, CF₃, CF₂H, CH₂F, OCH₃, OCD₃, or C(═O)CH₃. In some cases, each further substituent independently is D, Br, Cl, F, OH, CH₃, CH(CH₃)₂, CF₃, CF₂H, CFH₂, OCH₃, OCD₃, N(CH₃)₂, (C═O)CH₃,

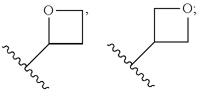 [SMILES: *C1CCO1 ; *C1COC1]

or two geminal further substituents, together with the atom to which they are attached, form

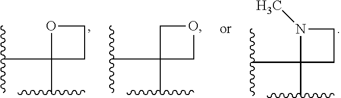

In some cases, the C₀₋₂alkylene-heterocycloalkyl is

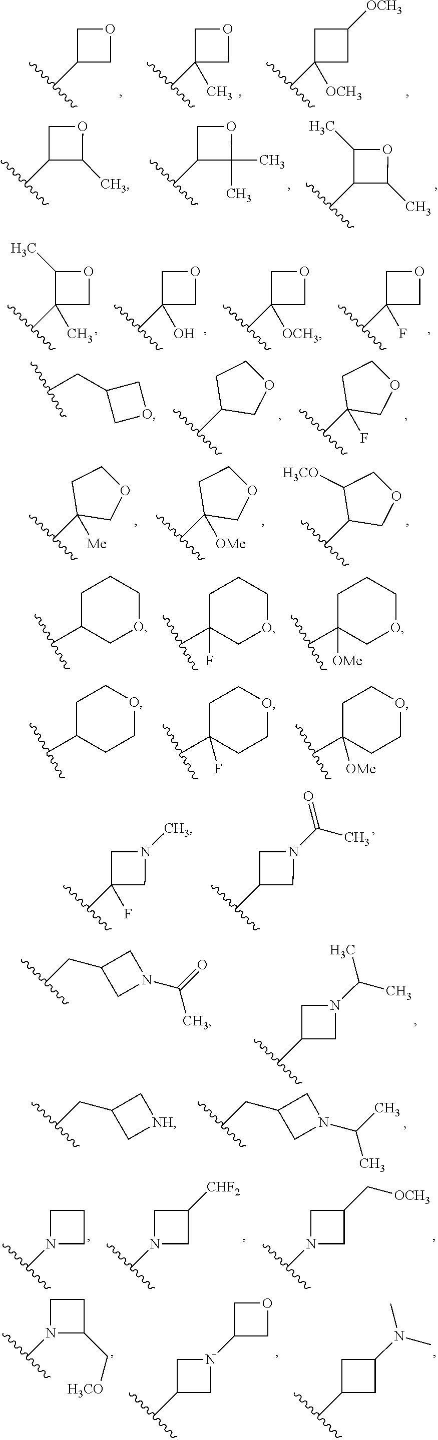

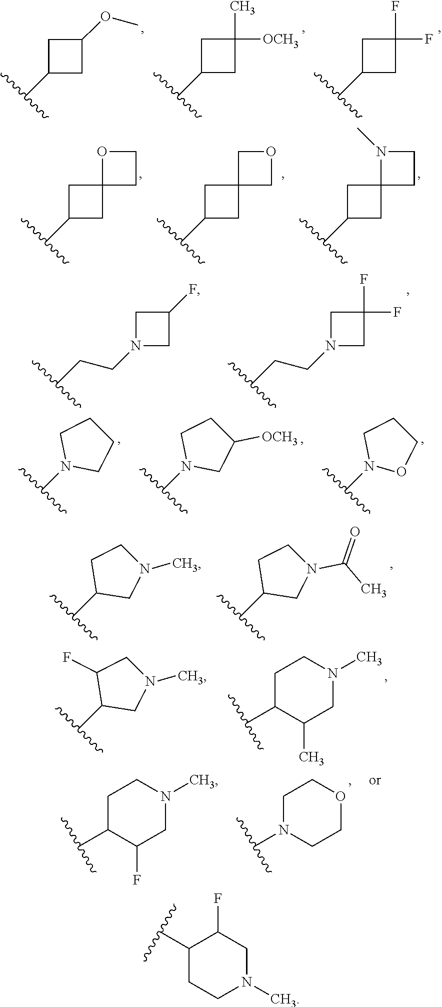

In some cases, the C₀₋₂alkylene-phenyl is phenyl or CH₂-phenyl, wherein each of the foregoing independently is optionally substituted with 1 or more further substituents. In some cases, each substituent of the heteroaryl of Z independently is Br, Cl, F, CN, CF₃, CHF₂, CH₂F, CH₂CHF₂, CH₂CH₂F, CH(CH₂F)₂, CH(CH₃)CH₂F, CH(CH₃)CHF₂, C(═CH₂)CH₂F, OH, CH₂OH, CH₂CH₂OH, CH(CH₃)CH₂OH, C(CH₃)₂OH, C(CH₃)₂CH₂OH, CH₂C(CH₃)₂OH, NH₂, CH₂NH₂, CH₂NHCH₃, CH₂N(CH₃)₂, CH₂CH₂NH₂, CH₂CH₂NHCH₃, CH₂CH₂N(CH₃)₂, C₁₋₆alkyl selected from CH₃, CH₂CH₃, CH₂CH₂CH₃, and CH(CH₃)₂, C₂₋₆alkenyl selected from CH═CH₂, CH₂CH═CH₂, and CH═CHCH₃, C₀₋₆alkylene-C₁₋₃alkoxy selected from OCH₃, CH₂OCH₃, CH₂CH₂OCH₃, CH₂CH₂OCH₂CH₃, CH₂CH₂CH₂OCH₃, CH(CH₃)OCH₃, CH(CH₃)CH₂OCH₃, CH(OCH₃)CH₂OCH₃, CH(CH₃)(OCH₃)CH₂OCH₃, C(CH₃)₂OCH₃,C(CH₃)₂CH₂OCH₃, CH₂CH(CH₃)OCH₃, CH₂(CH₃)(OCH₃)OCH₃, CH₂C(CH₃)₂OCH₃, and CH₂C(CH₃)₂OCH₃, cycloalkyl selected from cyclopropyl, cyclobutyl, cyclopentyl, and cyclohexyl, or heterocycloalkyl selected from azetidinyl, pyrrolidinyl, piperidinyl, pyrazolidinyl, oxetanyl, tetrahydrofuranyl, tetrahydropyranyl, isoxazolidinyl, and morpholinyl; wherein each of the C₁₋₆alkyl, C₂₋₆alkenyl, C₀₋₆alkylene-C₁₋₃alkoxy, cycloalkyl, and heterocycloalkyl substituents independently is substituted with 1-3 further substituents and each further substituent independently is D, halo, C₁₋₃alkyl, C₁₋₃haloalkyl, C₁₋₂alkyleneOH, C₁₋₂alkylene-C₁₋₃alkoxy, C₁₋₃deuterated alkoxy, N(R^{N1})₂, (C═O) C₁₋₃alkyl, C₃₋₅cycloalkyl, heterocycloalkyl having 3-5 total ring atoms and 1 or 2 heteroatoms selected from N, O, and S; or two geminal further substituents, together with the atom to which they are attached, form spiro-C₃₋₅cycloalkyl, or spiro-heterocycloalkyl having 3-5 total ring atoms and 1 or 2 heteroatoms selected from N, O, and S; or two vicinal further substituents, together with the atoms to which they are attached, form fused-C₃₋₅cycloalkyl or fused-heterocycloalkyl having 3-5 total ring atoms and 1 or 2 heteroatoms selected from N, O, and S. In some cases, each further substituent independently is D, Br, Cl, F, OH, CH₃, CF₃, CF₂H, CFH₂, OCH₃, OCD₃, CH₂OCH₃, N(CH₃)₂, (C═O)CH₃, oxetanyl, azetidinyl, or two geminal further substituents, together with the atom to which they are attached, form spiro-oxetanyl or spiro-azetidinyl; wherein each of the foregoing oxetanyl, azetidinyl, spiro-oxetanyl, and spiro-azetidinyl independently is unsubstituted or substituted with F, CH₃, or a combination thereof. In some cases, each further substituent independently is D, Br, Cl, F, OH, CH₃, CF₃, CF₂H, CFH₂, OCH₃, CH₂OCH₃, OCD₃, N(CH₃)₂, (C═O)CH₃,

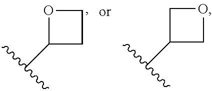 [SMILES: *C1CCO1 ; *C1COC1]

or two geminal substituents, together with the atom to which they are attached, form

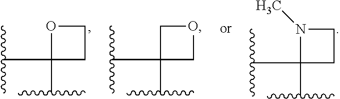

In some cases, each substituent of the heteroaryl of Z independently is Cl, F, CN, CH₃, CD₃, CH₂CH₃, CH(CH₃)₂, CF₃, CHF₂, CH₂F, CH₂CHF₂, CH₂CH₂F, CH(CH₂F)₂, CH(CH₃)CH₂F, CH(CH₃)CHF₂, C(═CH₂)CH₂F, OH, CH₂OH, CH₂CH₂OH, CH(CH₃)CH₂OH, C(CH₃)₂OH, C(CH₃)₂CH₂OH, CH₂C(CH₃) OH, OCH₃, OCD₃, CH₂OCH₃, CH₂OCD₃, CH₂CH₂OCH₃, CHFCH₂OCH₃, CF₂CH₂OCH₃, CH₂CH₂OCD₃, CH—CH₂OCH₂CH₃,CH₂CH₂CH₂OCH₃, CH₂CH₂CH₂OCD₃, CH(CH₃)OCH₃, CH(CH₃)CH₂OCH₃, CH(CH₃)CH₂OCD₃, C(CH₃)₂CH₂OCH₃, C(CH₃)₂CH₂OCD₃, CH₂CH(CH₃)OCH₃, CH₂CH(CH₃)OCD₃, CH₂C(CH₃)₂OCH₃, CH₂C(CH₃),OCD₃, NH₂, CH₂NH₂, CH₂NHCH₃, CH₂N(CH₃)₂, CH₂CH₂NH₂, CH—CH₂NHCH₃, CH—CH₂N(CH₃)₂,

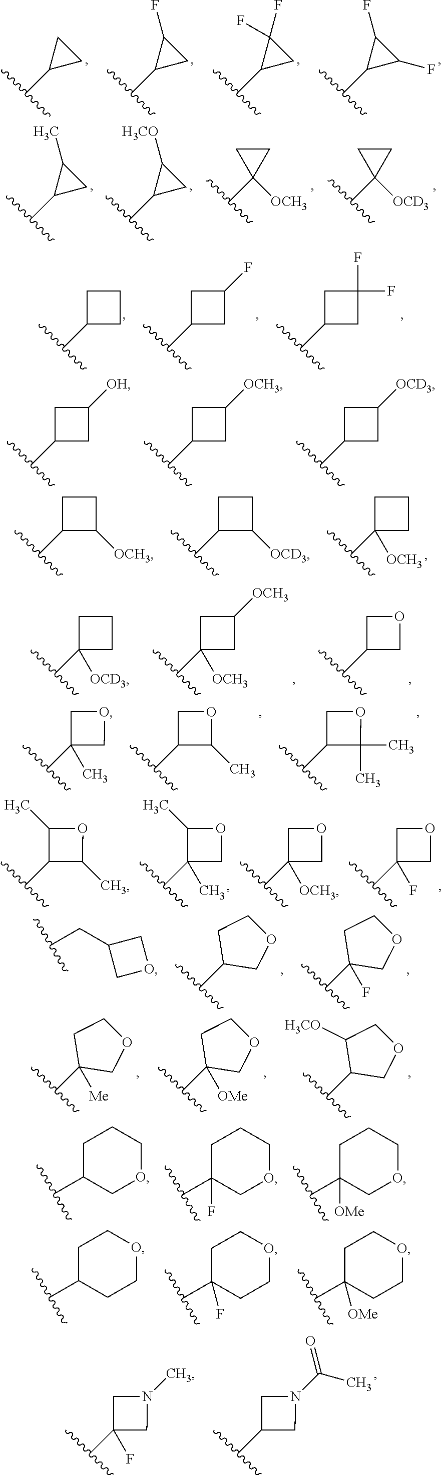

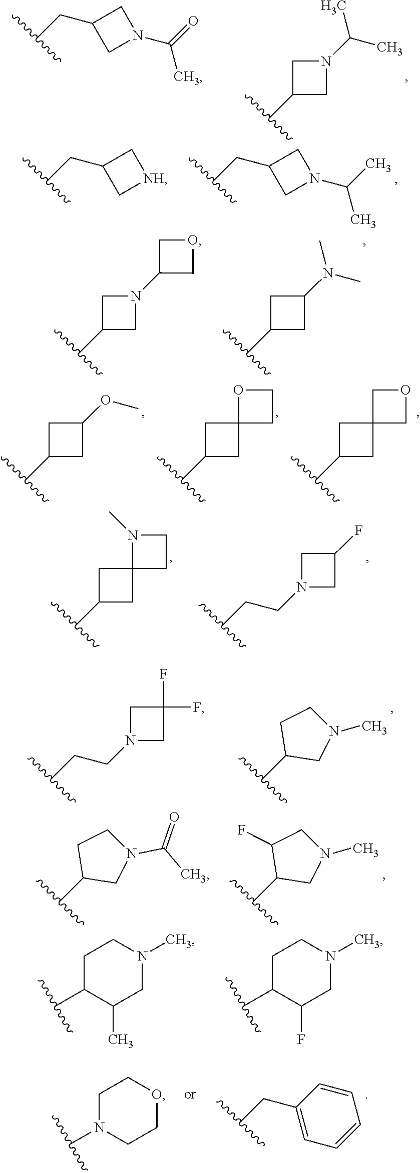

In some cases, each substituent of the heteroaryl of Z independently is CH₃, C(CH₃)₂CH₂OH, CH₂CH₂OCH₃, CH₂CH₂OCD₃, CH(CH₃)OCH₃,

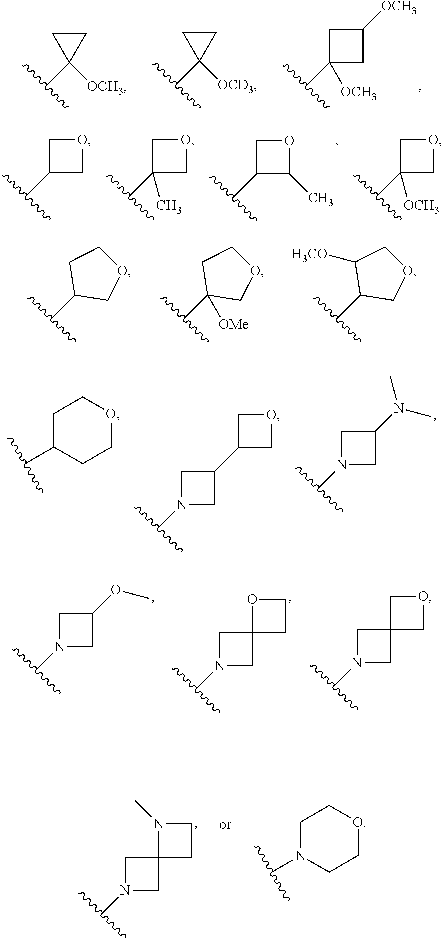

In some cases, each substituent of the heteroaryl of Z independently is CH₃, C(CH₃)₂CH₂OH, CH₂CH₂OCH₃, CH₂CH₂OCD₃, CH(CH₃)OCH₃,

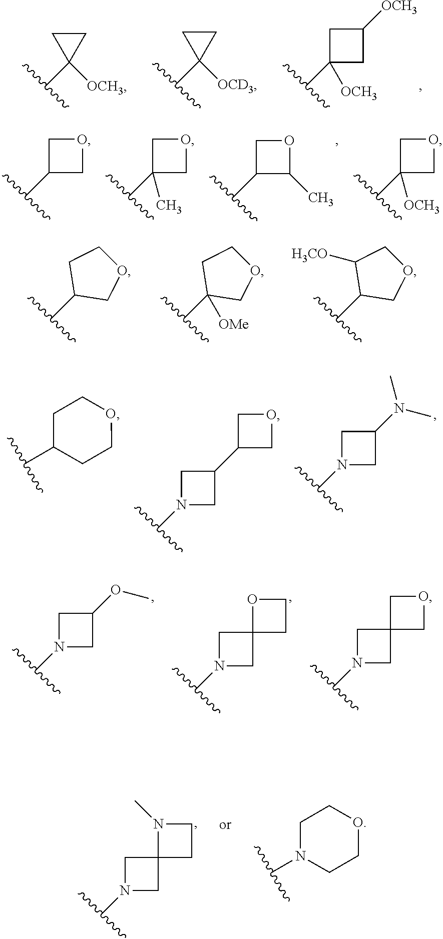

In some cases, each substituent of the heteroaryl of Z independently is CH₃, CH₂CH₂OCH₃,

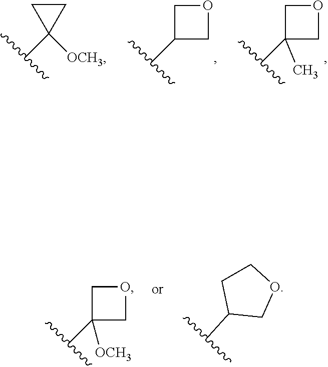

In some cases, Z is heteroaryl that is substituted with CH₃ and CH₂CH₂OCH₃,

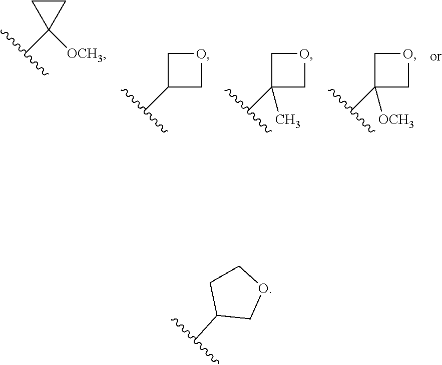

In some cases, the heteroaryl group has 2 substituents selected from CH₃, CH₂CH₂OCH₃,

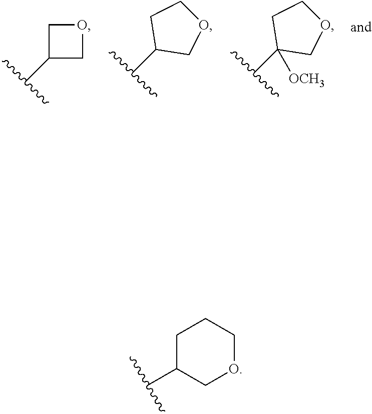 [SMILES: *C1COC1 ; *C1CCOC1 ; *C1(OC)CCOC1 ; *C1CCCOC1]

In some cases, Z is heteroaryl and has a structure

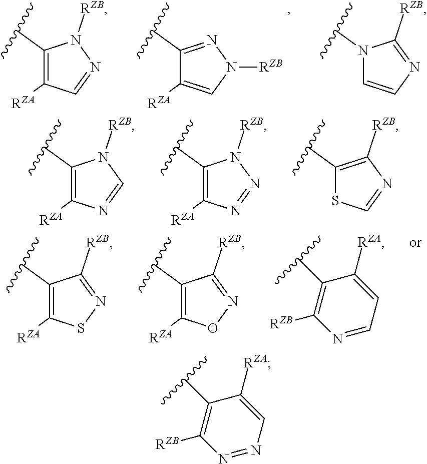

wherein each of R^{ZA} and R^{ZB} is as defined herein for the substituents of the heteroaryl group of Z. In some cases, Z is heteroaryl and has a structure

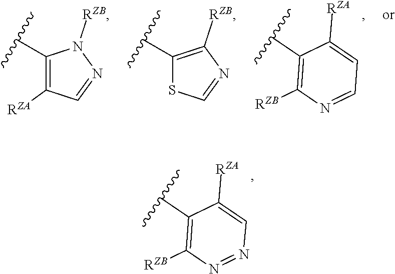

wherein each of R^{ZA} and R^{ZB} is as defined herein for the substituents of the heteroaryl group of Z. In some cases, Z is

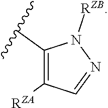

In some cases, Z is

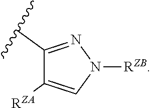

In some cases, Z is

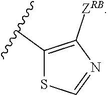

In some cases, Z is

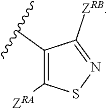

In some cases, Z is

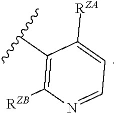

In some cases, Z is

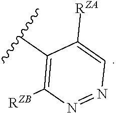

In some cases, each of R^{ZA} and R^{ZB} independently is halo, CN, C₁₋₃alkyl, C₁₋₆haloalkyl, C₂₋₆alkenyl, C₂₋₆haloalkenyl, C₀₋₆alkylene-OH, C₀₋₆alkylene-C₁₋₃alkoxy, C₀₋₆alkylene-N(R^{N1})₂, C₀₋₂alkylene-C₃₋₆cycloalkyl, C₀₋₂alkylene-heterocycloalkyl having 3-6 total ring atoms and 1-3 heteroatoms selected from N, O, and S, or C₀₋₁alkylene-phenyl; wherein each of the C₁₋₆alkyl, C₂₋₆alkenyl, C₀₋₃alkylene-C₁₋₃alkoxy, C₃₋₇cycloalkyl, heterocycloalkyl, and phenyl substituents independently is optionally substituted with 1 or more further substituents, and each R^{N1} independently is H or C₁₋₃alkyl. In some cases, each of the C₁₋₃alkyl, C₂₋₆alkenyl, C₀₋₆alkylene-C₁₋₃alkoxy, cycloalkyl, heterocycloalkyl, and phenyl substituents independently is unsubstituted or substituted with 1-3 further substituents, and each further substituent independently is D, halo, C₁₋₃alkyl, C₁₋₃haloalkyl, C₁₋₂alkyleneOH, C₁₋₂alkylene-C₁₋₄alkoxy, C₁₋₃deuterated alkoxy, N(R^{N1})₂. (C═O)C₁₋₃alkyl, C₃₋₄cycloalkyl, heterocycloalkyl having 3-5 total ring atoms and 1 or 2 heteroatoms selected from N, O, and S; or two geminal further substituents, together with the atom to which they are attached, form spiro-C₃₋₅cycloalkyl, or spiro-heterocycloalkyl having 3-5 total ring atoms and 1 or 2 heteroatoms selected from N, O, and S; or two vicinal further substituents, together with the atoms to which they are attached, form fused-C₃₋₅cycloalkyl or fused-heterocycloalkyl having 3-5 total ring atoms and 1 or 2 heteroatoms selected from N, O, and S; wherein each of the foregoing cycloalkyl and heterocycloalkyl further substituents independently is unsubstituted or substituted with 1 or 2 substituents, and each substituent independently is halo or C₁₋₃alkyl. In some cases, each of R^{ZA} and R^{ZB} independently is Cl, F, CN, CH₃, CD₃, CH₂CH₃, CH(CH₃)₂, CF₃, CHF₂ CH₂F, CH₂CHF₂, CH₂CH₂F, CH(CH₂F)₂, CH(CH₃)CH₂F, CH(CH₃)CHF₂, C(═CH₂)CH₂F, OH, CH₂OH, CH₂CH₂OH, CH(CH₃)CH₂OH, C(CH₃)₂OH, C(CH₃)₂CH₂OH, CH₂C(CH₃)₂OH, OCH₃, OCD₃, CH₂OCH₃, CH₂OCD₃, CH₂CH₂OCH₃, CHFCH₂OCH₃, CF₂CH₂OCH₃, CH₂CH₂OCD₃, CH₂CH₂OCH₂CH₃,CH₂CH₂CH₂OCH₃, CH₂CH₂CH₂OCD₃, CH(CH₃)OCH₃, CH(CH₃)CH₂OCH₃, CH(CH₃)CH₂OCD₃, C(CH₃)₂CH₂OCH₃, C(CH₃)₂CH₂OCD₃, CH₂CH(CH₃)OCH₃, CH₂CH(CH₃)OCD₃, CH₂C(CH₃)₂OCH₃, CH₂C(CH₃)₂OCD₃, NH₂, CH₂NH₂, CH₂NHCH₃, CH₂N(CH₃)₂, CH—CH₂NH₂, CH₂CH₂NHCH₃, CH₂CH₂N(CH₃)₂,

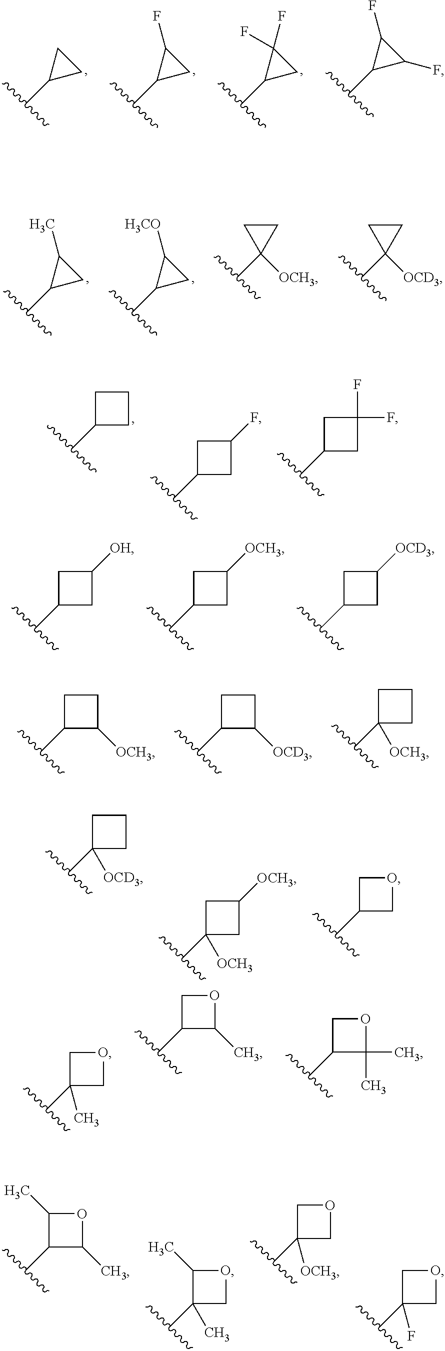

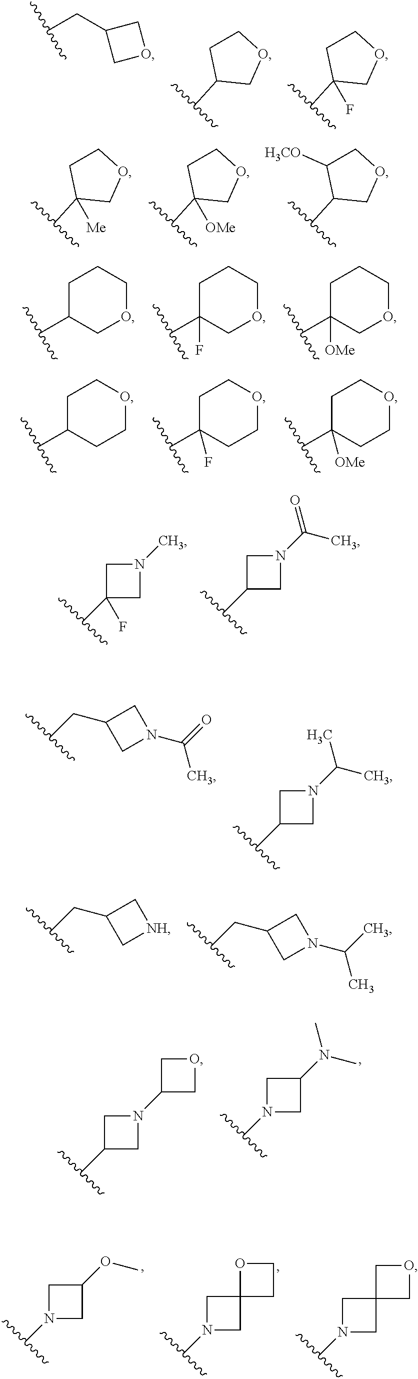

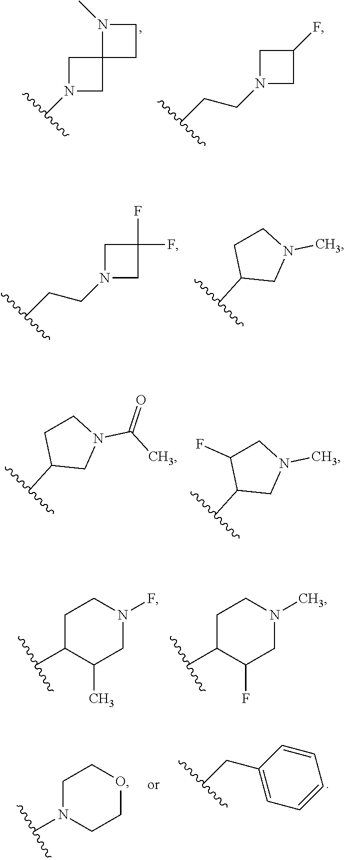

In some cases, R^{ZA} is Cl, F, CH₃, CH₂CH₃, CH(CH₃)₂, CF₃, CHF₂, CH₂F, CH₂CHF₂, or CH₂CH₂F; and R^{ZB} is CH₃, CH₂CH₂OCH₃, CF₂CH₂OCH₃, CH₂CH₂OCD₃, CH₂CH₂CH₂OCH₃, CH₂CH(CH₃)OCH₃, CH₂C(CH₃)₂OCH₃,

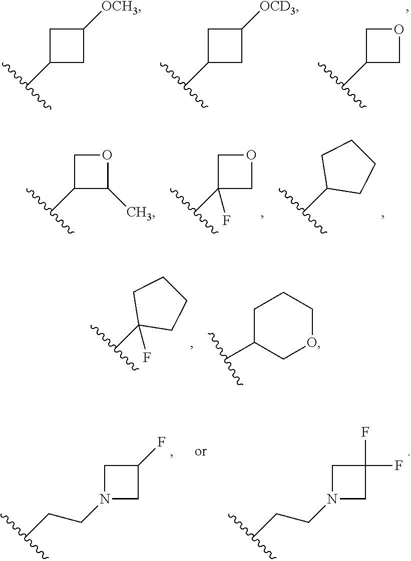

In some cases, R^{ZA} is CH₃; and R^{ZB} is CH₃, CH₂CH₂OCH₃, CF₂CH₂OCH₃, CH—CH₂OCD₃, CH₂CH₂CH₂OCH₃, CH₂CH(CH₃)OCH₃, CH₂C(CH₃)₂OCH₃,

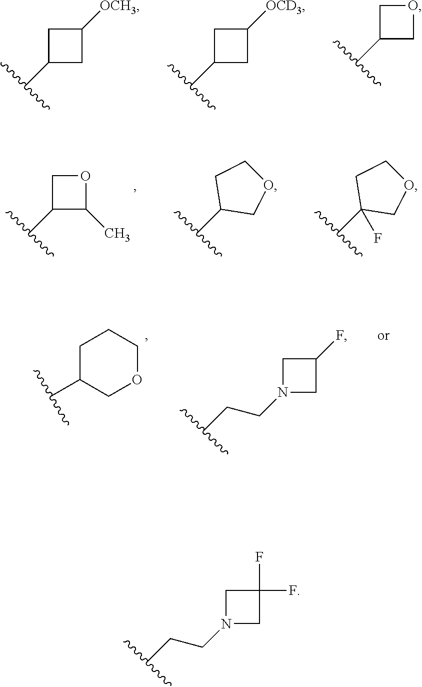

In some cases, R^{ZA} is CH₃; and R^{ZB} is CH₃, C(CH₃)₂CH₂OH, CH₂CH₂OCH₃, CH₂CH₂OCD₃, CH(CH₃)OCH₃,

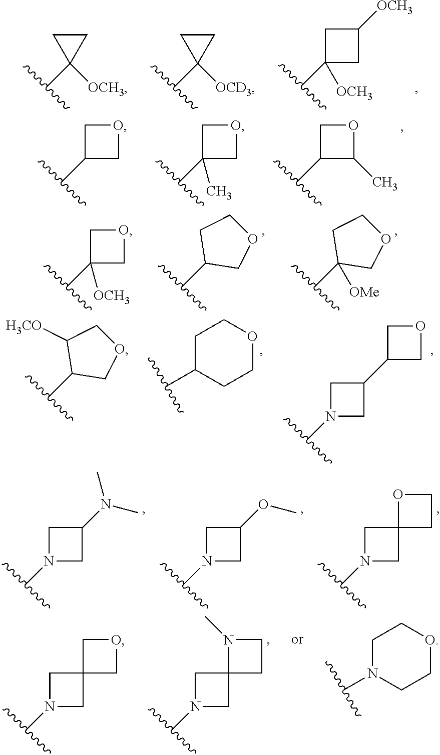

In some cases, R^{ZA} is CH₃; and R²⁸ is CH₃, CH₂CH₂OCH₃,

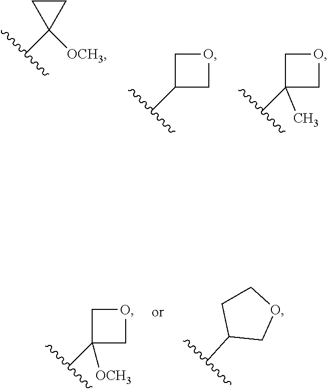

In some cases, Z is

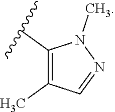 [SMILES: *c1c(C)cnn1C]

In some cases, Z is

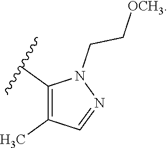 [SMILES: *c1c(C)cnn1CCOC]

In some cases, Z is

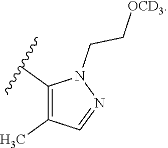

In some cases, Z is

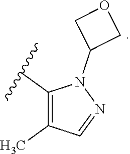 [SMILES: *c1c(C)cnn1C1COC1]

In some cases, Z is

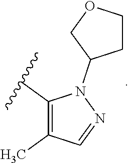 [SMILES: *c1c(C)cnn1C1CCOC1]

In some cases, Z is

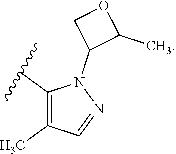 [SMILES: *c1c(C)cnn1C1COC1C]

In some cases. Z is

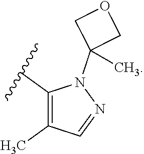 [SMILES: *c1c(C)cnn1C1(C)COC1]

some cases, Z is

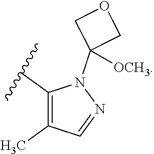 [SMILES: *c1c(C)cnn1C1(OC)COC1]

### In

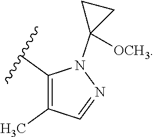 [SMILES: *c1c(C)cnn1C1(OC)CC1]

some cases, Z is

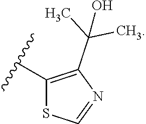 [SMILES: *c1scnc1C(C)(C)O]

In some cases, Z is

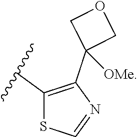 [SMILES: *c1scnc1C1(OC)COC1]

In some cases, Z is

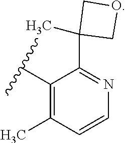 [SMILES: *c1c(C)ccnc1C1(C)COC1]

In some cases, Z is

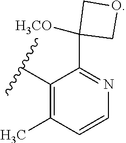 [SMILES: *c1c(C)ccnc1C1(OC)COC1]

In some cases, Z is a bicyclic ring comprising a heteroaryl ring having 5 or 6 total ring atoms and 1-3 heteroatoms selected from N, O, and S fused to C₅₋₆cycloalkyl or a heterocycloalkyl ring having 5 or 6 total ring atoms and 1 or 2 heteroatoms selected from N, O, and S; wherein the bicyclic ring is unsubstituted or substituted with 1-4 substituents. In some cases, the heteroaryl 1 ring of the bicyclic ring is pyridyl, pyridazinyl, pyrimidinyl, or pyrazinyl; the cycloalkyl ring of the bicyclic ring is cyclopentyl or cyclohexyl; and the heterocycloalkyl ring of the bicyclic ring is pyrrolidinyl, tetrahydrofuranyl, tetrahydropyranyl, or tetrahydrothiophenyl. In some cases, the heteroaryl group is pyridyl and the heterocycloalkyl group is furanyl. In some cases, Z is a bicyclic ring comprising heteroaryl having 5 or 6 total ring atoms and 1-3 heteroatoms selected from N, O, and S fused to a ring having 5 or 6 total ring atoms and 0, 1, or 2 heteroatoms selected from N, O, and S; wherein the bicyclic ring is unsubstituted or substituted with 1 or more substituents, such as 1-4 substituents, or 1-3 substituents, or 1-2 substituents, or 1 substituent. In some cases, the heteroaryl of the bicyclic ring is pyridyl, pyridazinyl, pyrimidinyl, or pyrazinyl; and the fused ring has 5 total atoms and 1 oxygen atom in the fused ring, 5 total atoms and 1 nitrogen atom in the fused ring, 6 total atoms and 1 nitrogen or oxygen atom in the ring, or 6 total atoms, 1 oxygen atom, and 1 nitrogen atom in the fused ring. In some cases, the heteroaryl group is pyridyl and the fused ring has 5 total atoms and 1 oxygen atom in the fused ring. In some cases, the heteroaryl group is imidazolyl or pyrazolyl and the fused ring has 5 total atoms and 1 nitrogen atom in the fused ring, 6 total atoms and 1 nitrogen or oxygen atom in the ring, or 6 total atoms, 1 oxygen atom, and 1 nitrogen atom in the fused ring. In some cases, the bicyclic ring is unsubstituted. In some cases, the bicyclic ring is substituted with 1-4 substituents, and each substituent independently is halo, CN, C₁₋₆alkyl, C₁₋₆haloalkyl, C₀₋₆alkylene-OH, or C₀₋₃alkylene-C₁₋₃alkoxy. In some cases, each substituent of the bicyclic ring independently is Br, Cl, F, CN, CH₃, CH₂CH₃, CH₂CH₂CH₃, CH(CH₃)>, CF₃, CHF₂, CH₂F, CH₂CHF₂, CH₂CH₂F, CH(CH₂F)₂, CH(CH₃)CH₂F, CH(CH₃)CHF₂), OH, CH₂OH, CH₂CH₂OH, CH(CH₃)CH₂OH, C(CH₃)₂OH, C(CH₃)₂CH₂OH, CH₂C(CH₃)₂OH, OCH₃, CH₂OCH₃, CH₂CH₂OCH₃, CH₂CH₂CH₂OCH₃, CH(CH₃)CH₂OCH₃, C(CH₃)₂CH₂OCH₃, CH₂CH(CH₃)OCH₃, or CH₂C(CH₃)₂OCH₃. In some cases, each substituent of the bicyclic ring independently is Cl, Br, F, CH₃, OH, CH₂OH, OCH₃, or CH₂OCH₃. In some cases, Z is

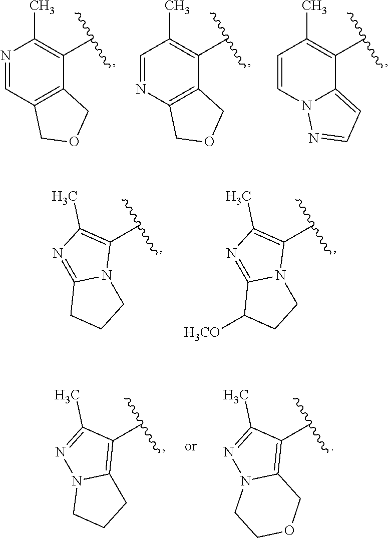

In some cases, the disclosure provides a compound of Formula (II):

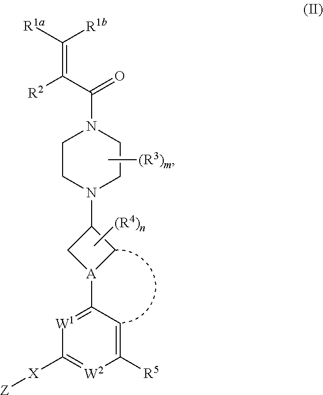

or a pharmaceutically acceptable salt thereof,
wherein:
m is 0, 1, 2, 3, or 4;
n is 0, 1, or 2;
A is N, CH, C-halo, C—CN, C—C₁₋₃alkyl, C—C₁₋₃haloalkyl, C—C₀₋₂alkyleneOH, or C—C₀₋₃alkylene-C₁₋₄alkoxy;
each of W¹ and W² independently is N, CH, C-halo, C—CN, C—C₁₋₃alkyl, C—C₂₋₃alkenyl, C—C₂₋₃alkynyl, C—C₁₋₃haloalkyl, C—C₀₋₃alkyleneOH, or C—C₀₋₃alkylene-C₁₋₄alkoxy, wherein each of the alkenyl and alkynyl is unsubstituted or substituted with 1-3 substituents and each substituent independently is halo, C₁₋₃haloalkyl, C₀₋₃alkyleneOH, or C₀₋₃alkyleneC₁₋₄alkoxy;
X is heterocycloalkyl or heterocycloalkenyl, each having 4-7 total ring atoms and 1-3 heteroatoms selected from N, O, and S, wherein each of the heterocycloalkyl and heterocycloalkenyl is unsubstituted or substituted with 1-3 substituents, and each substituent independently is halo, C₁₋₃alkyl, C₁₋₃haloalkyl, C₀₋₂alkyleneOH, C₀₋₂alkyleneC₁₋₃alkoxy, or C₀₋₂alkyleneCN;
Z is phenyl, heteroaryl comprising 5 or 6 total ring atoms and 1-3 heteroatoms selected from N, O, and S, or a bicyclic ring comprising a heteroaryl having 5 or 6 total ring atoms and 1-3 heteroatoms selected from N, O, and S fused to C₅₋₆cycloalkyl or a heterocycloalkyl ring having 5 or 6 total ring atoms and 1 or 2 heteroatoms selected from N, O, and S;
   wherein each of the phenyl, heteroaryl, and bicyclic ring is unsubstituted or substituted with 1-4 substituents, and each substituent independently is halo, CN, C₁₋₆alkyl, C₁₋₆haloalkyl, C₂₋₆alkenyl, C₂₋₆haloalkenyl, C₀₋₆alkylene-OH, C₀₋₆alkylene-C₁₋₃alkoxy, C₀₋₆alkylene-N(R^{N1})₂, C₀₋₂alkylene-C₃₋₆cycloalkyl, C₀₋₂alkylene-heterocycloalkyl having 3-6 total ring atoms and 1-3 heteroatoms selected from N, O, and S, or C₀₋₂alkylene-phenyl;
   wherein each of the C₁₋₆alkyl, C₂₋₆alkenyl, C₀₋₃alkylene-C₁₋₃alkoxy, cycloalkyl, heterocycloalkyl, and phenyl substituents independently is unsubstituted or substituted with 1-3 further substituents, and each further substituent independently is D, halo, C₁₋₄alkyl, C₁₋₃haloalkyl, C₁₋₂alkyleneOH, C₃₋₅alkylene-C₁₋₃alkoxy, C₁₋₃deuterated alkoxy, N(R^{N1})₂, (C═O)C₁₋₃alkyl, C₃₋₅cycloalkyl, or heterocycloalkyl having 3-5 total ring atoms and 1 or 2 heteroatoms selected from N, O, and S; or two geminal further substituents, together with the atom to which they are attached, form spiro-C₃₋₅cycloalkyl or spiro-heterocycloalkyl having 3-5 total ring atoms and 1 or 2 heteroatoms selected from N, O, and S; or two vicinal further substituents, together with the atoms to which they are attached, form fused-C₃₋₅cycloalkyl or fused-heterocycloalkyl having 3-5 total ring atoms and 1 or 2 heteroatoms selected from N, O, and S;
   wherein each of the foregoing cycloalkyl and heterocycloalkyl further substituents independently is unsubstituted or substituted with 1 or 2 substituents, and each substituent independently is halo or C₁₋₃alkyl;
[symbol] is C₂₋₆alkylene, C₂₋₆alkenylene, heteroalkylene having 2-6 total atoms and 1-3 heteroatoms selected from N, O, and S, or heteroalkenylene having 3-6 total atoms and 1 or 2 heteroatoms selected from N, O, and S, wherein [symbol] is unsubstituted or substituted with 1-4 substituents, and each substituent independently is C₁₋₃alkyl, C₁₋₃haloalkyl, C₂₋₃alkenyl, halo, CN, C₀₋₃alkyleneOH, C₀₋₆alkylene-C₁₋₃alkoxy, C₃₋₃cycloalkyl, C₄₋₅cycloalkenyl, heterocycloalkyl having 4 or 5 total ring atoms and 1-3 heteroatoms selected from N, O, and S, heterocycloalkenyl having 4 or 5 total ring atoms and 1-3 heteroatoms selected from N, O, and S, or phenyl; or two geminal substituents, together with the atom to which they are attached, form oxo, =CH₂, spiro-C₃₋₅cycloalkyl, spiro-C₄ cycloalkenyl, spiro-heterocycloalkyl having 3-5 total ring atoms and 1 or 2 heteroatoms selected from N, O and S, or spiro-heterocycloalkenyl having 4 or 5 total ring atoms and 1 or 2 heteroatoms selected from N, O and S; or two vicinal substituents, together with the atoms to which they are attached, form fused-C₃₋₅cycloalkyl, fused-C₄₋₅cycloalkenyl, fused-heterocycloalkyl having 3-5 total ring atoms and 1 or 2 heteroatoms selected from N, O and S or fused-heterocycloalkenyl having 4 or 5 total ring atoms and 1 or 2 heteroatoms selected from N, O and S;
each of R^{1a}, R^{1b}, and R² independently is H, D, halo, C₁₋₄alkyl, C₁₋₄haloalkyl, C₁₋₂alkylene-OH, C₀₋₂alkylene-C₁₋₄alkoxy, C₁₋₂alkylene-C₁₋₄haloalkoxy, C₀₋₂alkylene-CN, C₀₋₂alkylene-N(R^{N1})₂, C₁₋₂alkylene-heterocycloalkyl having 3-6 total ring atoms and 1 or 2 heteroatoms selected from N, O, and S, or R^{1b} and R², together with the carbon atoms to which they are attached, form

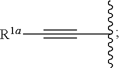

each R³ independently is C₁₋₃alkyl, C₁₋₃haloalkyl,

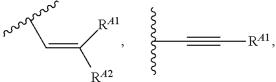

C₀₋₂alkyleneCN, C₀₋₃alkyleneOH, or C₀₋₃alkylene-C₁₋₄alkoxy; two geminal R³, together with the atom to which they are attached, form oxo, spiro-C₃₋₇cycloalkyl, spiro-C₄₋₇-cycloalkenyl, spiro-heterocycloalkyl having 3-7 total ring atoms and 1 or 2 heteroatoms selected from N, O, and S, or spiro-heterocycloalkenyl having 4-7 total ring atoms and 1 or 2 heteroatoms selected from N, O, and S; or two vicinal R³, together with the atoms to which they are attached, form fused-C₃₋₇cycloalkyl, fused-C₄cycloalkenyl, fused-heterocycloalkyl having 3-7 total ring atoms and 1 or 2 heteroatoms selected from N, O, and S, or fused-heterocycloalkenyl having 4-7 total ring atoms and 1 or 2 heteroatoms selected from N, O, and S; or

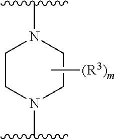

is deuterated;
each R⁴ independently is C₁₋₃alkyl, C₁₋₃haloalkyl, C₀₋₃alkyleneCN, C₁₋₃alkyleneOH, or C₁₋₃alkylene-C₁₋₃alkoxy; or two geminal R⁴, together with the atom to which they are attached, form oxo, spiro-C₃₋₇cycloalkyl, or spiro-heterocycloalkyl having 4-7 total ring atoms and 1 or 2 heteroatoms selected from N, O and S;
R⁵ is halo, C₁₋₃haloalkyl, C₁₋₆alkyl, C₂₋₄alkenyl, C₂₋₄alkynyl, C₁₋₃alkoxy, C₁₋₅thioalkyl, C₃₋₇cycloalkyl, C₅₋₇cycloalkenyl, heterocycloalkyl having 3-7 total ring atoms and 1-3 heteroatoms selected from N, O, and S, or heterocycloalkenyl having 5-7 total ring atoms and 1-3 heteroatoms selected from N, O, and S, wherein each of the foregoing independently is unsubstituted or substituted with 1-3 substituents, and each substituent independently is C₁₋₅haloalkyl, C₀₋₃alkylene-OH, C₀₋₆alkylene-C₁₋₃alkoxy, C₃₋₇cycloalkyl, C₅₋₇cycloalkenyl, heterocycloalkyl having 3-7 total ring atoms and 1-3 heteroatoms selected from N, O, and S, heterocycloalkenyl having 4-7 total ring atoms and 1-3 heteroatoms selected from N, O, and S, or phenyl;
each of R^{A1} and R^{A2} independently is H, C₁₋₃alkyl, C₁₋₃haloalkyl, or C₃₋₅cycloalkyl; and
each R^{N1} independently is H or C₁₋₄alkyl.

In some cases, m is 0 or 1; n is 0; o is 0 or 1; A is N; W¹ is CH; W² is N; X is

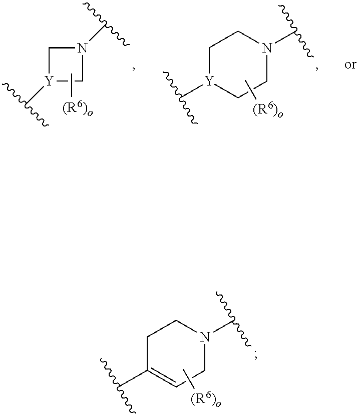

Y is CH or N; and Z is heteroaryl comprising 5 or 6 total ring atoms and 1-3 heteroatoms selected from N, O, and S, wherein the heteroaryl is substituted with 1 or more substituents; each of R^{1a}, R^{1b}, and R² is H; R³ is CH₃; and R⁵ is CH₂F, CHF₂, or CF₃, In some cases,

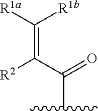

is

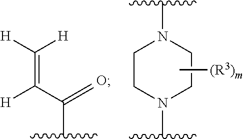

is

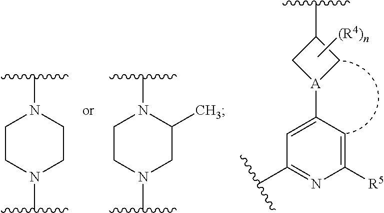

is

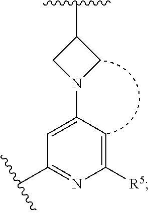

R⁵ is CHF₂ or CF₃; X is

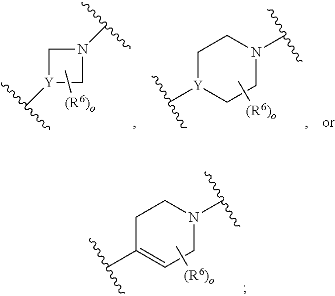

o is 0 or 1; R⁶ is CH₃; Y is CH or N; and Z is pyrazolyl or pyridyl, each of which is substituted with 1 or 2 substituents, and each substituent independently is Cl, F. CN, CH₃, CD₃, CH₂CH₃, CH(CH₃)₂, CF₃, CHF₂, CH₂F, CH₂CHF₂, CH₂CH₂F, CH(CH₂F)₂, CH(CH₃)CH₂F, CH(CH,)CHF₂, C(═CH₂)CH₂F, OH, CH₂OH, CH—CH₂OH, CH(CH₃)CH₂OH, C(CH₃)₂OH, C(CH₃)₂CH: OH, CH₂C(CH₃)₂OH, OCH₃, OCD₃, CH₂OCH₃, CH₂OCD₃, CH₂CH₂OCH₃, CHFCH₂OCH₃, CF₂CH₂OCH₃, CH₂CH₂OCD₃. CH₂CH₂OCH₂CH₃, CH₂CH₂CH₂OCH₃, CH₂CH₂CH₂OCD₃, CH(CH₃)OCH₃, CH(CH₃)CH₂OCH₃, CH(OCH₃)CH₂OCH₃, CH(CH₃)(OCH₃)CH₂OCH₃, CH(CH₂F) (CH₃)CH₂OCD₃, CH(CH₃)CH₂OCD₃, C(CH₃)₂OCH₃, C(CH₃)₂CH₂OCH₃, C(CH₃)₂CH₂OCD₃, CH₂CH(CH₃)OCH₃, CH₂(CH₃)(OCH₃)OCH₃, CH₂CH(CH₃)OCD₃, CH₂C(CH₃)₂OCH₃, CH₂C(CH₃)₂OCD₃, NH₂, CH₂NH₂, CH₂NHCH₃, CH₂N(CH₃)₂, CH₂CH₂NH₂, CH₂CH₂NHCH₃, CH₂CH₂N(CH₃)₂,

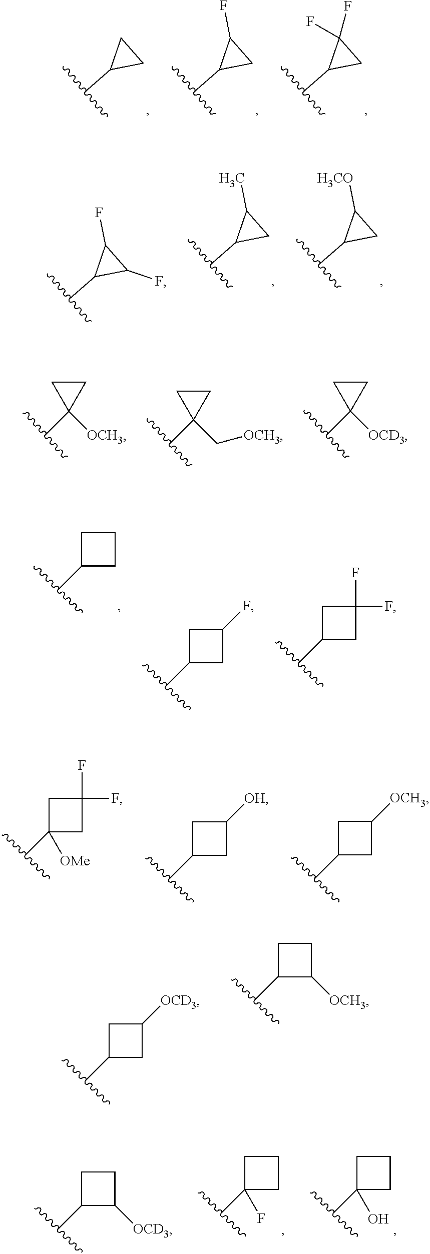

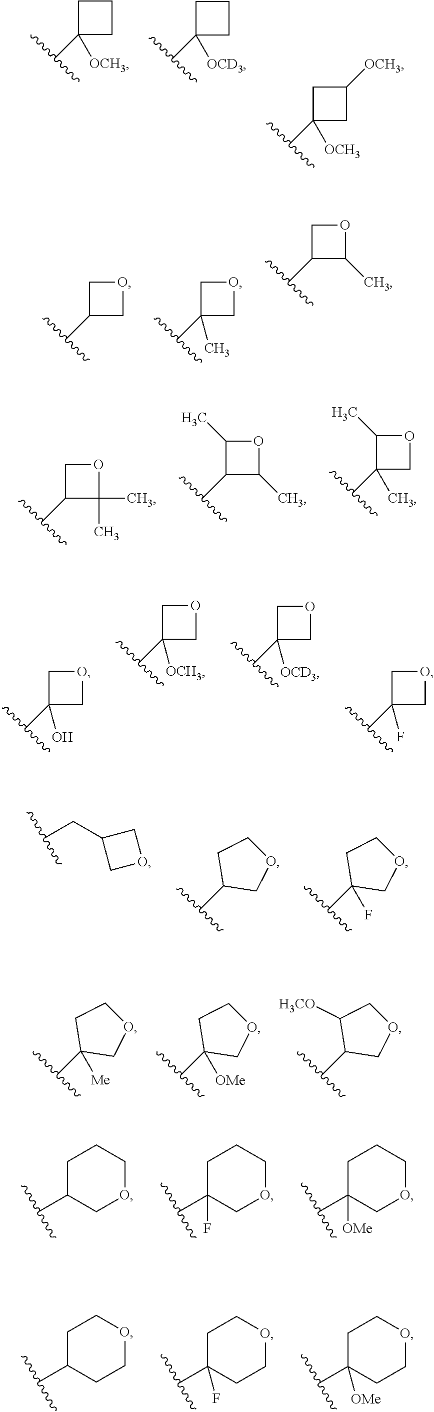

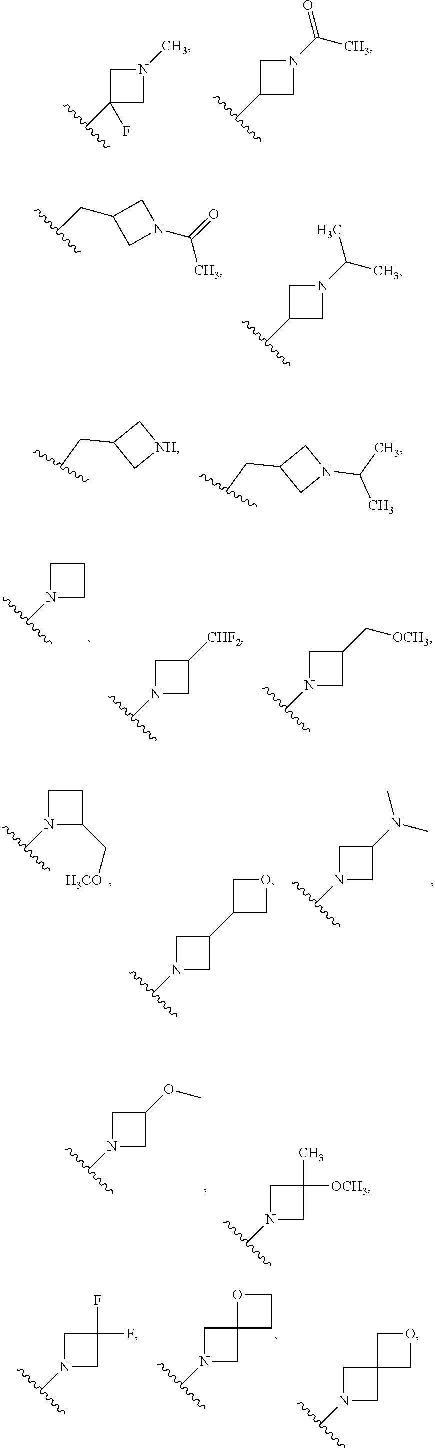

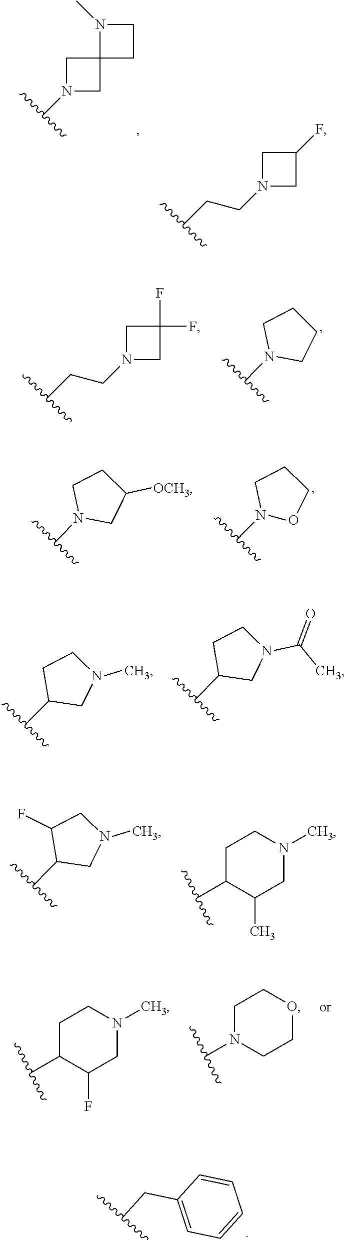

In some cases, X is

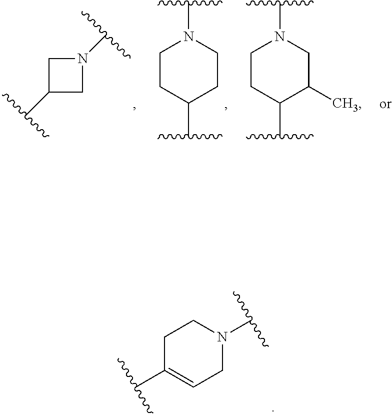

In some cases, X is

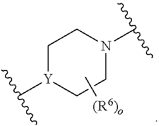

In some cases, X is

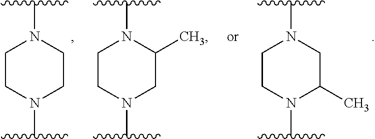

In some cases, [symbol] is

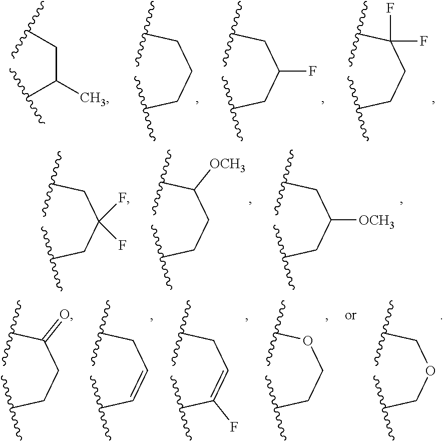

In some cases, [symbol] is

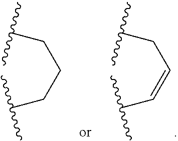 [SMILES: *CCC* ; */C=C\C*]

In some cases, the heteroaryl of Z is pyrazolyl, thiazolyl, pyridyl or pyridazinyl, wherein each of the foregoing is substituted with 1 or 2 substituents. In some cases, the heteroaryl of Z is pyrazolyl or pyridyl, and each of the foregoing is substituted with 2 substituents. In some cases, each substituent independently is C₁₋₆alkyl, C₀₋₂alkylene-C₃₋₆cycloalkyl, C₀₋₂alkylene-heterocycloalkyl having 3-6 total ring atoms and 1-3 heteroatoms selected from N, O, and S, or a combination of the foregoing, wherein the cycloalkyl and heterocycloalkyl is optionally substituted with 1 or 2 further substituents, and each further substituent independently is D, CH₃, OCH₃, OCD₃, N(CH₃)₂,

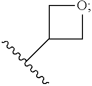 [SMILES: *C1COC1]

or two geminal further substituents, together with the atom to which they are attached, form

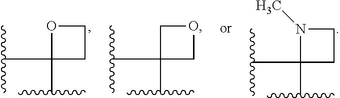

In some cases, Z is

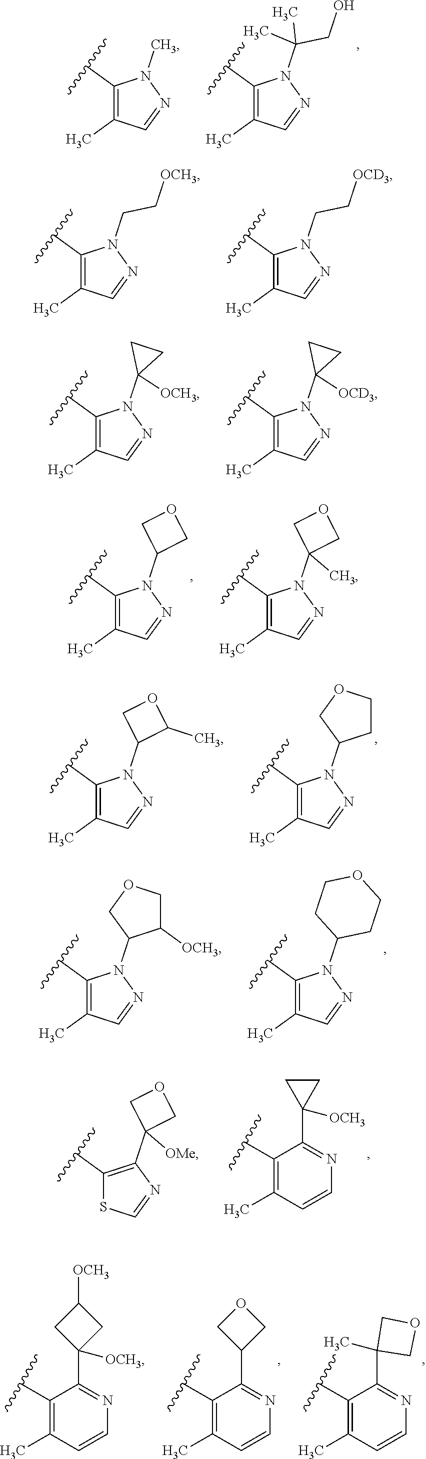

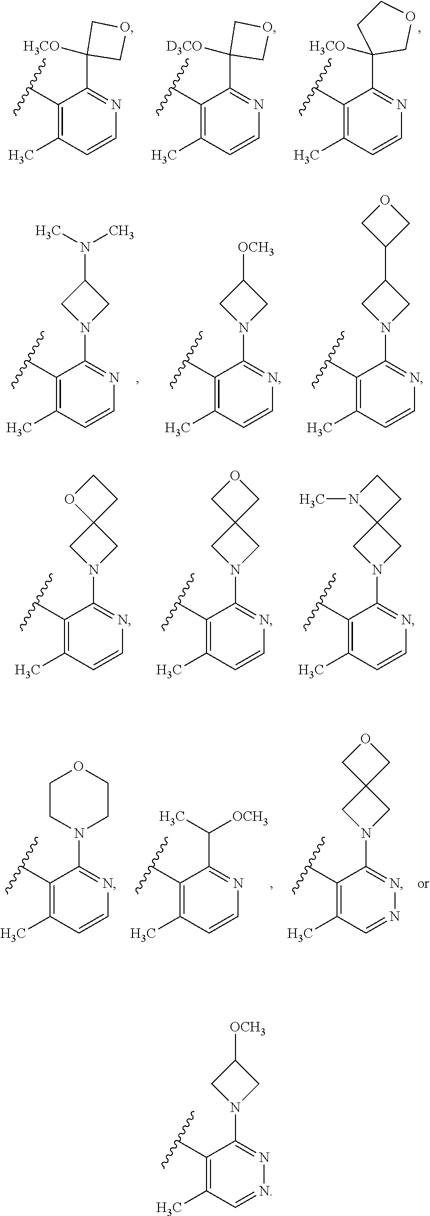

In some cases, Z is

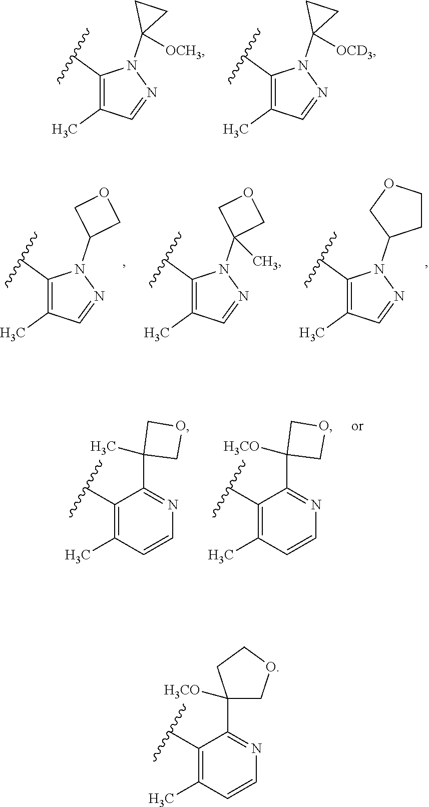

In some cases, Z is

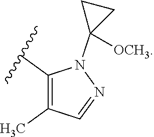 [SMILES: *c1c(C)cnn1C1(OC)CC1]

In some cases, Z is

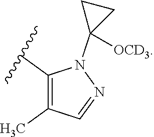

In some cases, Z is

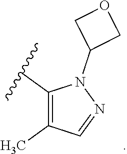 [SMILES: *c1c(C)cnn1C1COC1]

In some cases, Z is

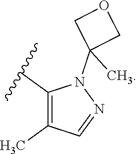 [SMILES: *c1c(C)cnn1C1(C)COC1]

In some cases, Z is

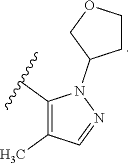 [SMILES: *c1c(C)cnn1C1CCOC1]

In some cases, Z is

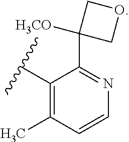 [SMILES: *c1c(C)ccnc1C1(OC)COC1]

In some cases, Z is

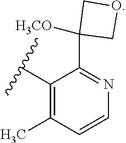 [SMILES: *c1c(C)ccnc1C1(OC)COC1]

In some cases, Z is

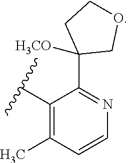 [SMILES: *c1c(C)ccnc1C1(OC)CCOC1]

In some cases,

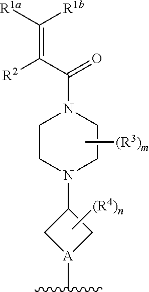

### of Formula (II) is

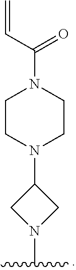 [SMILES: *N1CC(N2CCN(C(=O)C=C)CC2)C1]

In some cases, Formula (II) has a structure of Formula (IIA):

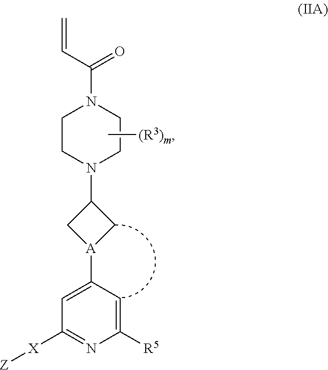

or a pharmaceutically acceptable salt thereof. In some cases, Formula (II) has a structure of Formula (IIB), or Formula (IIC), or Formula (IID), or Formula (IIE), or Formula (IIF):

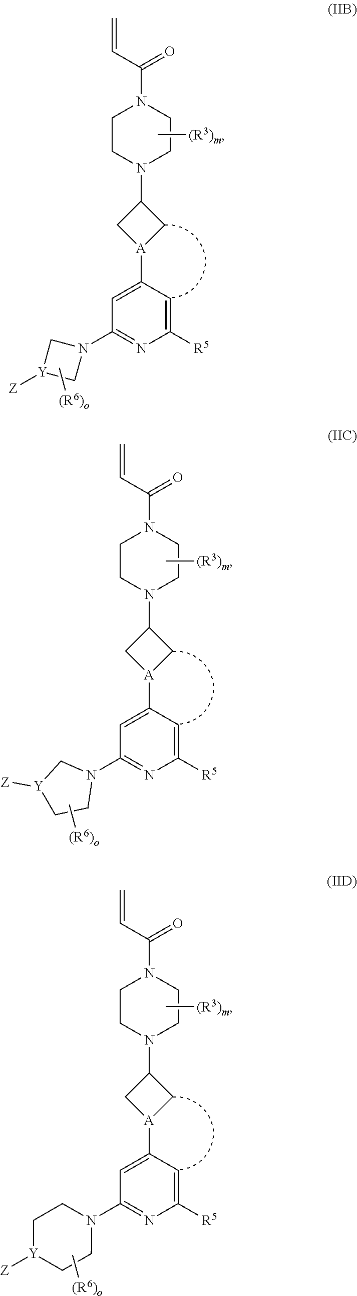

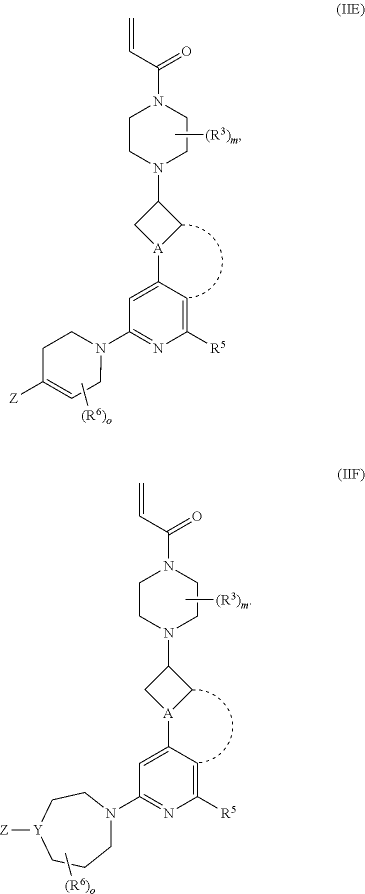

or a pharmaceutically acceptable salt of any of the foregoing.

In some cases,

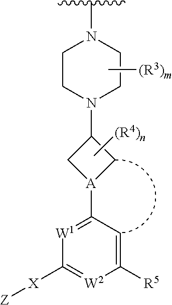

exhibits the stereochemical configuration:

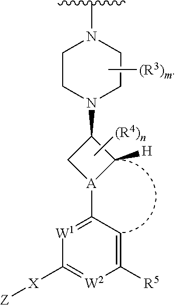

In some cases,

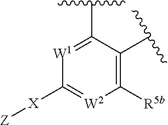

is

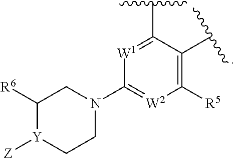

In some cases,

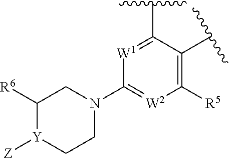

exhibits the following stereochemical configuration:

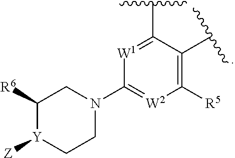

In some cases,

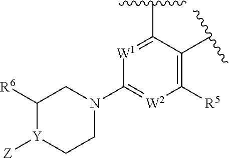

exhibits the following stereochemical configuration:

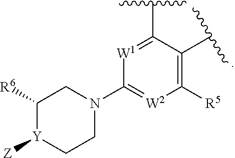

In some cases, Formula (II) exhibits the stereochemical configuration shown in Formula (II′):

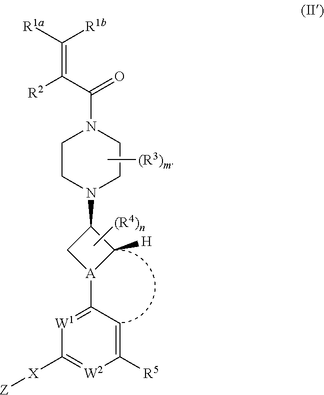

In some cases, the compound of Formula (II) is a compound as listed in Table A, or a pharmaceutically acceptable salt thereof:

TABLE A

| Chemical Structure | Name |
|---|---|
| 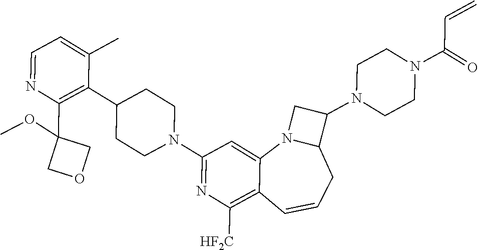 | 1-(4-(-4-(difluoromethyl)-2-(4-(2-(3- methoxy-3-oxetanyl)-4-methyl-3- pyridinyl)-1-piperidinyl)-7,7a,8,9- tetrahydroazeto[1,2-a]pyrido[3,4-f] azepin-8-yl)-1-piperazinyl)-2-propen- 1-one |
| 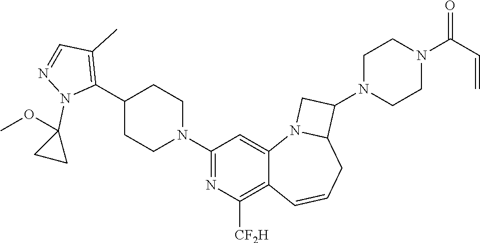 [SMILES: C=CC(=O)N1CCN(C2CN3c4cc(N5CCC(c6c(C)cnn6C6(OC)CC6)CC5)nc(C(F)F)c4C=CCC23)CC1] | 1-(4-(-4-(difluoromethyl)-2-(4-(1-(1- methoxycyclopropyl)-4-methyl-1H- pyrazol-5-yl)-1-piperidinyl)-7,7a,8,9- tetrahydroazeto[1,2-a]pyrido[3,4-f] azepin-8-yl)-1-piperazinyl)-2-propen- 1-one |
| 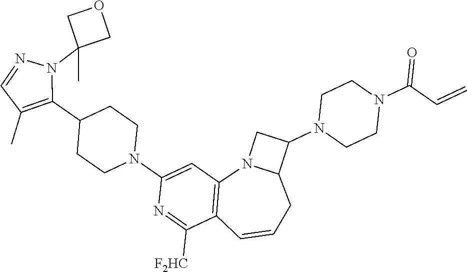 [SMILES: C=CC(=O)N1CCN(C2CN3c4cc(N5CCC(c6c(C)cnn6C6(C)COC6)CC5)nc(C(F)F)c4C=CCC23)CC1] | 1-(4-(-4-(difluoromethyl)-2-(4-(4- methyl-1-(3-methyl-3-oxetanyl)-1H- pyrazol-5-yl)-1-piperidinyl)-7,7a,8,9- tetrahydroazeto[1,2-a]yrido[3,4-f] azepin-8-yl)-1-piperazinyl)-2-propen- 1-one |
| 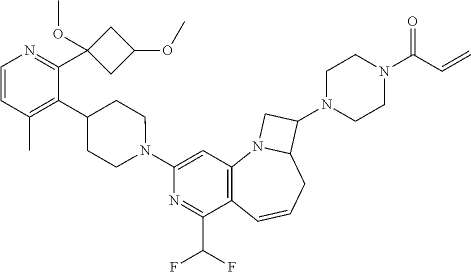 [SMILES: C=CC(=O)N1CCN(C2CN3c4cc(N5CCC(c6c(C)ccnc6C6(OC)CC(OC)C6)CC5)nc(C(F)F)c4C=CCC23)CC1] | 1-(4-(-4-(difluoromethyl)-2-(4-(2- (1,3-dimethoxycyclobutyl)-4-methyl- 3-pyridinyl)-1-piperidinyl)-7,7a,8,9- tetrahydroazeto[1,2-a]pyrido(3,4-f] azepin-8-yl)-1-piperazinyl)-2-propen- 1-one |
| 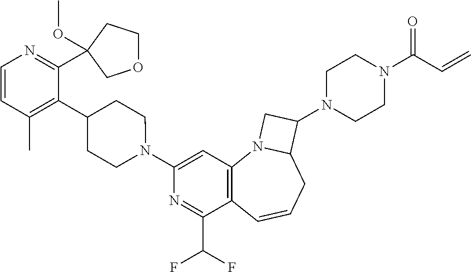 [SMILES: C=CC(=O)N1CCN(C2CN3c4cc(N5CCC(c6c(C)ccnc6C6(OC)CCOC6)CC5)nc(C(F)F)c4C=CCC23)CC1] | 1-(4-(4-(difluoromethyl)-2-(4-(2-(3- methoxytetrahydro-3-furanyl)-4- methyl-3-pyridinyl)-1-piperidinyl)- 7,7a,8,9-tetrahydroazeto[1,2-a]pyrido [3,4-f]azepin-8-yl)-1-piperazinyl)-2- propen-1-one |
| 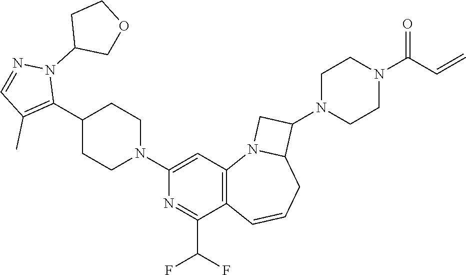 [SMILES: C=CC(=O)N1CCN(C2CN3c4cc(N5CCC(c6c(C)cnn6C6CCOC6)CC5)nc(C(F)F)c4C=CCC23)CC1] | 1-(4-(4-(difluoromethyl)-2-(3-methyl- 4-(4-methyl-1-(tetrahydro-3-furanyl)- 1H-pyrazol-5-yl)-1-piperidinyl)-7,7a, 8,9-tetrahydroazeto[1,2-a]pyrido[3,4- f]azepin-8-yl)-1-piperazinyl)-2- propen-1-one |
| 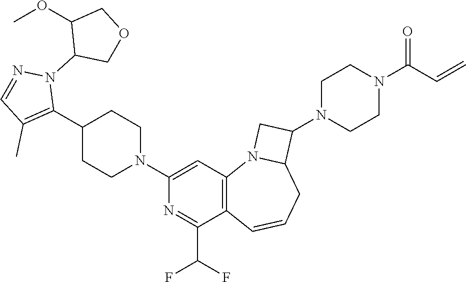 [SMILES: C=CC(=O)N1CCN(C2CN3c4cc(N5CCC(c6c(C)cnn6C6COCC6OC)CC5)nc(C(F)F)c4C=CCC23)CC1] | 1-(4-(4-(difluoromethyl)-2-(4-(1-(4- methoxytetrahydro-3-furanyl)-4- methyl-1H-pyrazol-5-yl)-1-piperidin- yl)-7,7a,8,9-tetrahydroazeto[1,2-a] pyrido[3,4-f]azepin-8-yl)-1-piperazin- yl)-2-propen-1-one |
| 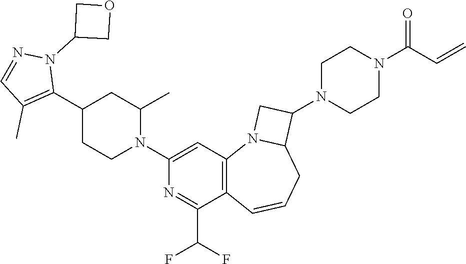 [SMILES: C=CC(=O)N1CCN(C2CN3c4cc(N5CCC(c6c(C)cnn6C6COC6)CC5C)nc(C(F)F)c4C=CCC23)CC1] | 1-(4-(4-(difluoromethyl)-2-(2-methyl- 4-(4-methyl-1-(3-oxetanyl)-1H- pyrazol-5-yl)-1-piperidinyl)-7,7a,8,9- tetrahydroazeto[1,2-a]pyrido[3,4-f] azepin-8-yl)-1-piperazinyl)-2-propen- 1-one |
| 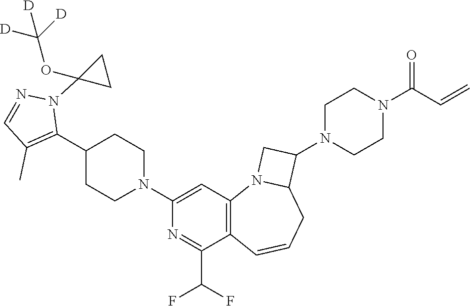 [SMILES: [2H]C([2H])([2H])OC1(n2ncc(C)c2C2CCN(c3cc4c(c(C(F)F)n3)C=CCC3C(N5CCN(C(=O)C=C)CC5)CN43)CC2)CC1] | 1-(4-(4-(difluoromethyl)-2-(4-(1-(1- (methoxy-d3)cyclopropyl)-4-methyl- 1H-pyrazol-5-yl)piperidin-1-yl)-7,7a, 8,9-tetrahydroazeto[1,2-a]pyrido[3,4- f]azepin-8-yl)piperazin-1-yl)-prop-2- en-1-one |
| 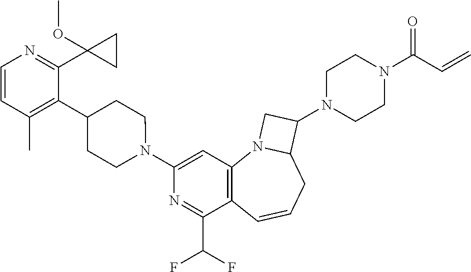 [SMILES: C=CC(=O)N1CCN(C2CN3c4cc(N5CCC(c6c(C)ccnc6C6(OC)CC6)CC5)nc(C(F)F)c4C=CCC23)CC1] | 1-(4-(4-(difluoromethyl)-2-(4-(2-(1- methoxycyclopropyl)-4-methyl-3- pyridinyl)-1-piperidinyl)-7,7a,8,9- tetrahydroazeto[1,2-a]pyrido[3,4-f] azepin-8-yl)-1-piperazinyl)-2-propen- 1-one |
| 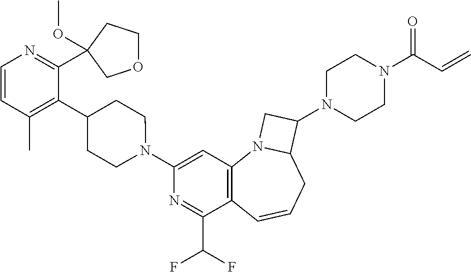 [SMILES: C=CC(=O)N1CCN(C2CN3c4cc(N5CCC(c6c(C)ccnc6C6(OC)CCOC6)CC5)nc(C(F)F)c4C=CCC23)CC1] | 1-(4-(4-(difluoromethyl)-2-(4-(2-(3- methoxytetrahydrofuran-3-yl)-4- methylpyridin-3-yl)piperidin-1-yl)- 7,7a,8,9-tetrahydroazeto[1,2a]pyrido [3,4-f]azepin-8-yl)piperazin-1-yl)- prop-2-en-1-one |
| 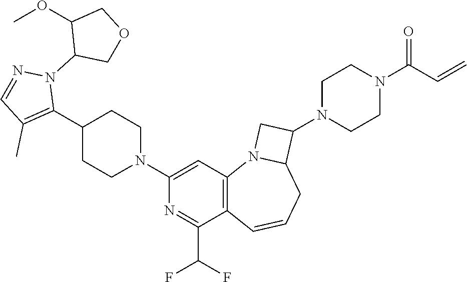 [SMILES: C=CC(=O)N1CCN(C2CN3c4cc(N5CCC(c6c(C)cnn6C6COCC6OC)CC5)nc(C(F)F)c4C=CCC23)CC1] | 1-(4-(4-(difluoromethyl)-2-(4-(1-(4- methoxytetrahydro-3-furanyl)-4- methyl-1H-pyrazol-5-yl)-1-piperidin- yl)-7,7a,8,9-tetrahydroazeto[1,2-a] pyrido[3,4-f]azepin-8-yl)-1-piperazin- yl)-2-propen-1-one |
| 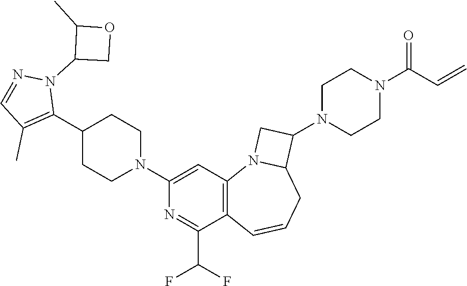 [SMILES: C=CC(=O)N1CCN(C2CN3c4cc(N5CCC(c6c(C)cnn6C6COC6C)CC5)nc(C(F)F)c4C=CCC23)CC1] | 1-(4-(4-(difluoromethyl)-2-(4-(4- methyl-1-(2-methyl-3-oxetanyl)-1H- pyrazol-5-yl)-1-piperidinyl)-7,7a,8,9- tetrahydroazeto[1,2-a]pyrido[3,4-f] azepin-8-yl)-1-piperazinyl)-2-propen- 1-one |
| 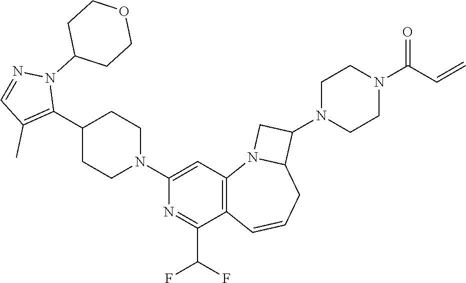 [SMILES: C=CC(=O)N1CCN(C2CN3c4cc(N5CCC(c6c(C)cnn6C6CCOCC6)CC5)nc(C(F)F)c4C=CCC23)CC1] | 1-(4-(4-(difluoromethyl)-2-(4-(4- methyl-1-(tetrahydro-2H-pyran-4-yl)- 1H-pyrazol-5-yl)-1-piperidinyl)-7,7a, 8,9-tetrahydroazeto[1,2-a]pyrido[3,4- f]azepin-8-yl)-1-piperazinyl)-2- propen-1-one |
| 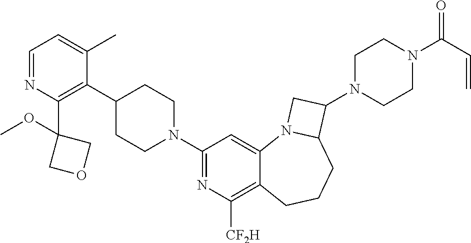 [SMILES: C=CC(=O)N1CCN(C2CN3c4cc(N5CCC(c6c(C)ccnc6C6(OC)COC6)CC5)nc(C(F)F)c4CCCC23)CC1] | 1-(4-(4-(difluoromethyl)-2-(4-(2-(3- methoxy-3-oxelanyl)-4-methyl-3- pyridinyl)-1-piperidinyl)-5,6,7,7a,8,9- hexahydroazeto[1,2-a]pyrido[3,4-f] azepin-8-yl)-1-piperazinyl)-2-propen- 1-one |
| 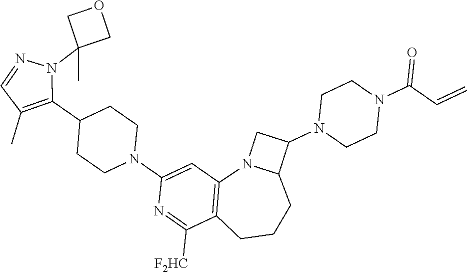 [SMILES: C=CC(=O)N1CCN(C2CN3c4cc(N5CCC(c6c(C)cnn6C6(C)COC6)CC5)nc(C(F)F)c4CCCC23)CC1] | 1-(4-(4-(difluoromethyl)-2-(4-(4- methyl-1-(3-methyloxetan-3-yl)- 1H-pyrazol-5-yl)piperidin-1-yl)- 5,6,7,7a,8,9-hexahydroazeto[1,2-a] pyrido[3,4-f]azepin-8-yl)piperazin- 1-yl)prop-2-en-1-one |
| 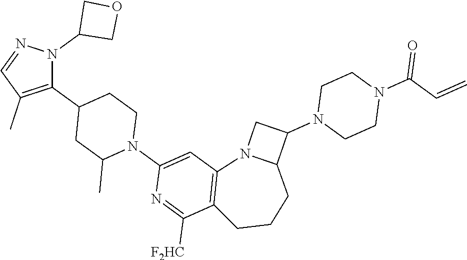 [SMILES: C=CC(=O)N1CCN(C2CN3c4cc(N5CCC(c6c(C)cnn6C6COC6)CC5C)nc(C(F)F)c4CCCC23)CC1] | 1-(4-(4-(difluoromethyl)-2-(2-methyl- 4-(4-methyl-1-(3-oxetanyl)-1H- pyrazol-5-yl)-1-piperidinyl)-5,6,7,7a, 8,9-hexahydroazeto[1,2-a]pyrido[3,4- f]azepin-8-yl)-1-piperazinyl)-2- propen-1-one |
| 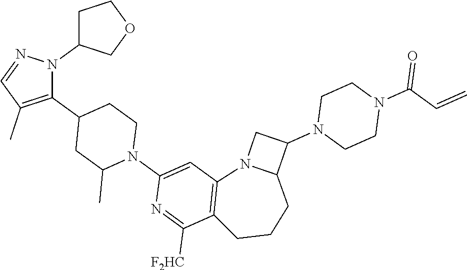 [SMILES: C=CC(=O)N1CCN(C2CN3c4cc(N5CCC(c6c(C)cnn6C6CCOC6)CC5C)nc(C(F)F)c4CCCC23)CC1] | 1-(4-(4-(difluoromethyl)-2-(2-methyl- 4-(4-methyl-1-(tetrahydro-3-furanyl)- 1H-pyrazol-5-yl)-1-piperidinyl)-5,6,7, 7a,8,9-hexahydroazeto[1,2-a]]pyrido [3,4-f]azepin-8-yl)-1-piperazin-yl)-2- propen-1-one |
| 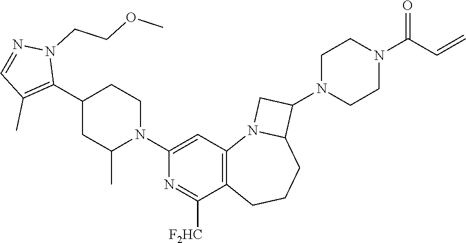 [SMILES: C=CC(=O)N1CCN(C2CN3c4cc(N5CCC(c6c(C)cnn6CCOC)CC5C)nc(C(F)F)c4CCCC23)CC1] | 1-(4-(4-(difluoromethyl)-2-(4-(1-(2- methoxyethyl)-4-methyl-1H-pyrazol- 5-yl)-2-methyl-1-piperidinyl)-5,6,7, 7a,8,9-hexahydroazeto[1,2-a]pyrido [3,4-f]azepin-8-yl)-1-piperazin-yl)-2- propen-1-one |
| 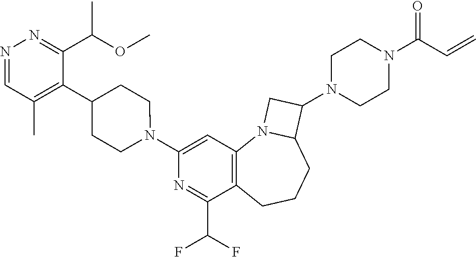 [SMILES: C=CC(=O)N1CCN(C2CN3c4cc(N5CCC(c6c(C)cnnc6C(C)OC)CC5)nc(C(F)F)c4CCCC23)CC1] | 1-(4-(4-(difluoromethyl)-2-(4-(3-(1- methoxyethyl)-5-methyl-4-pyridazin- yl)-1-piperidinyl)-5,6,7,7a,8,9-hexa- hydroazeto[1,2-a]pyrido[3,4-f]azepin- 8-yl)-1-piperazinyl)-2-propen-1-one |
| 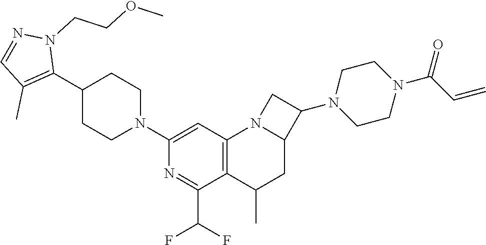 [SMILES: C=CC(=O)N1CCN(C2CN3c4cc(N5CCC(c6c(C)cnn6CCOC)CC5)nc(C(F)F)c4C(C)CC23)CC1] | 1-(4-(4-(difluoromethyl)-2-(4-(1-(2- methoxyethyl)-4-methyl-1H-pyrazol- 5-yl)-1-piperidinyl)-5-methyl-6,6a, 7,8-tetrahydro-5H-azeto[1,2-a][1,6] naphthyridin-7-yl)-1-piperazin-yl)-2- propen-1-one |
| 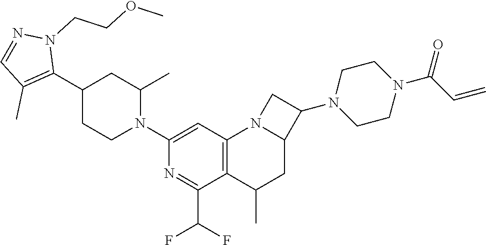 [SMILES: C=CC(=O)N1CCN(C2CN3c4cc(N5CCC(c6c(C)cnn6CCOC)CC5C)nc(C(F)F)c4C(C)CC23)CC1] | 1-(4-(4-(difluoromethyl)-2-(4-(1-(2- methoxyethyl)-4-methyl-1H-pyrazol- 5-yl)-2-methyl-1-piperidinyl)-5-meth- yl-6,6a,7,8-tetrahydro-5H-azeto[1,2- a][1,6]naphthyridin-7-yl)-1-piperazin- yl)-2-propen-1-one |
| 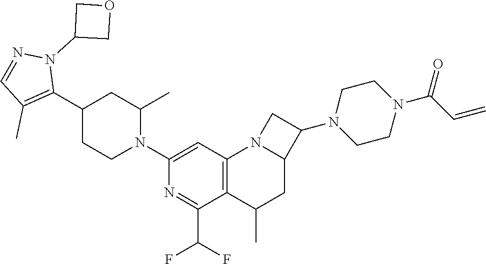 [SMILES: C=CC(=O)N1CCN(C2CN3c4cc(N5CCC(c6c(C)cnn6C6COC6)CC5C)nc(C(F)F)c4C(C)CC23)CC1] | 1-(4-(4-(difluoromethyl)-5-methyl-2- (2-methyl-4-(4-methyl-1-(3-oxetan- yl)-1H-pyrazol-5-yl)-1-piperidinyl)- 6,6a,7,8-tetrahydro-5H-azeto[1,2-a] [1,6]naphthyridin-7-yl)-]-piperazin- yl)-2-propen-1-one |
| 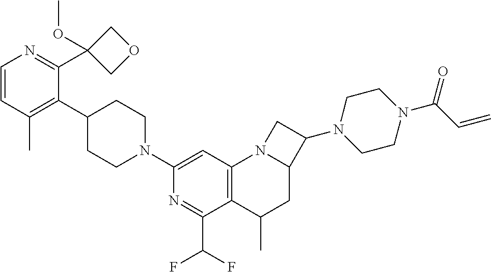 [SMILES: C=CC(=O)N1CCN(C2CN3c4cc(N5CCC(c6c(C)ccnc6C6(OC)COC6)CC5)nc(C(F)F)c4C(C)CC23)CC1] | 1-(4-(4-(difluoromethyl)-2-(4-(2-(3- methoxy-3-oxetanyl)-4-methyl-3- pyridinyl])-1-piperidinyl)-5-methyl- 6,6a,7,8-tetrahydro-5H-azeto[1,2-a] [1,6]naphthyridin-7-yl)-1-piperazin- yl)-2-propen-1-one |
| 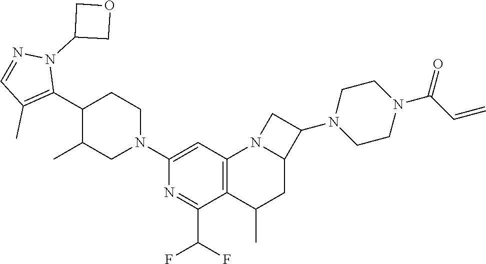 [SMILES: C=CC(=O)N1CCN(C2CN3c4cc(N5CCC(c6c(C)cnn6C6COC6)C(C)C5)nc(C(F)F)c4C(C)CC23)CC1] | 1-(4-(4-(difluoromethyl)-5-methyl-2- (3-methyl-4-(4-methyl-1-(3-oxetan- yl)-1H-pyrazol-5-yl)-1-piperidinyl)- 6,6a,7,8-tetrahydro-5H-azeto[1,2-a] [1,6]naphthyridin-7-yl)-1-piperazin- yl)-2-propen-1-one |
| 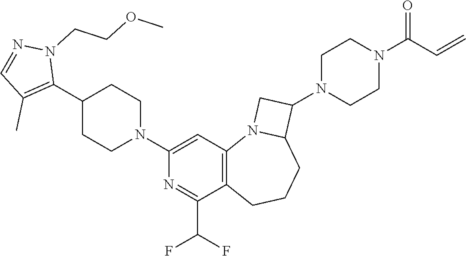 [SMILES: C=CC(=O)N1CCN(C2CN3c4cc(N5CCC(c6c(C)cnn6CCOC)CC5)nc(C(F)F)c4CCCC23)CC1] | 1-(4-(4-(difluoromethyl)-2-(4-(1-(2- methoxyethyl)-4-methyl-1H-pyrazol- 5-yl)-1-piperidinyl)-5,6,7,7a,8,9-hexa- hydroazeto[1,2-a]pyrido[3,4-f]azepin- 8-yl)-1-piperazinyl)-2-propen-1-one |
| 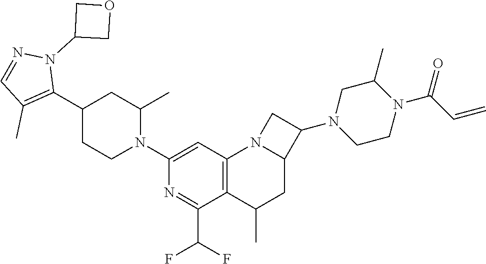 [SMILES: C=CC(=O)N1CCN(C2CN3c4cc(N5CCC(c6c(C)cnn6C6COC6)CC5C)nc(C(F)F)c4C(C)CC23)CC1C] | 1-(4-(4-(difluoromethyl)-5-methyl-2- (2-methyl-4-(4-methyl-1-(3-oxetan- yl)-1H-pyrazol-5-yl)-1-piperidinyl)- 6,6a,7,8-tetrahydro-5H-azeto[1,2-a] [1,6]naphthyridin-7-yl)-2-methyl-1- piperazinyl)-2-propen-1-one |
| 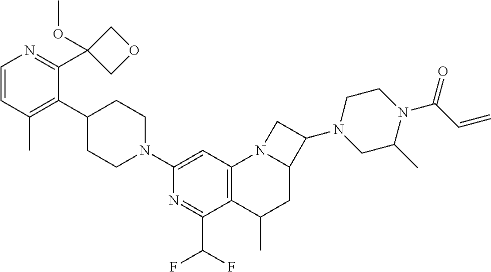 [SMILES: C=CC(=O)N1CCN(C2CN3c4cc(N5CCC(c6c(C)ccnc6C6(OC)COC6)CC5)nc(C(F)F)c4C(C)CC23)CC1C] | 1-(4-(4-(difluoromethyl)-2-(4-(2-(3- methoxy-3-oxetanyl)-4-methyl-3- pyridinyl)-1-piperidinyl)-5-methyl- 6,6a,7,8-tetrahydro-5H-azeto[1,2-a] [1,6]naphthyridin-7-yl)-2-methyl-1- piperazinyl)-2-propen-1-one |
| 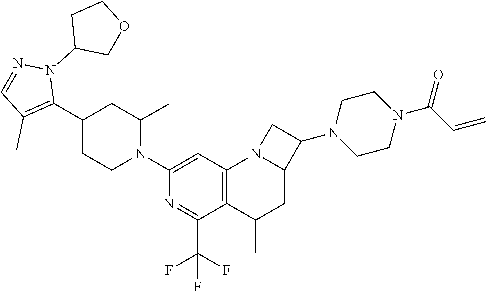 [SMILES: C=CC(=O)N1CCN(C2CN3c4cc(N5CCC(c6c(C)cnn6C6CCOC6)CC5C)nc(C(F)(F)F)c4C(C)CC23)CC1] | 1-(4-(5-methyl-2-(2-methyl-4-(4- methyl-1-(3-oxetanyl)-1H-pyrazol-5- yl)-1-piperidinyl)-4-(trifluoromethyl)- 6,6a,7,8-tetrahydro-5H-azeto[1,2-a] [1,6]naphthyridin-7-yl)-1-piperazin- yl)-2-propen-1-one |
| 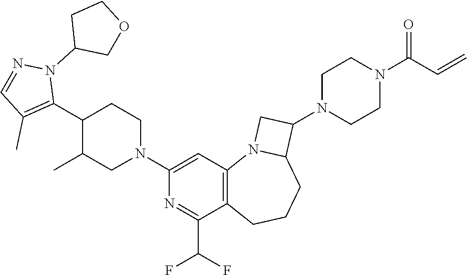 [SMILES: C=CC(=O)N1CCN(C2CN3c4cc(N5CCC(c6c(C)cnn6C6CCOC6)C(C)C5)nc(C(F)F)c4CCCC23)CC1] | 1-(4-(4-(difluoromethyl)-2-(3-methyl- 4-(4-methyl-1-((3S)-tetrahydro-3- furanyl)-1H-pyrazol-5-yl)-1-piperi- dinyl)-5,6,7,7a,8,9-hexahydroazeto [1,2-a]pyrido[3,4-f]azepin-8-yl)-1]- piperazinyl)-2-propen-1-one |
| 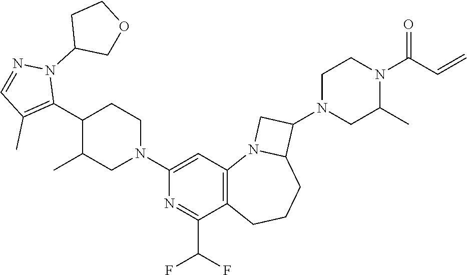 [SMILES: C=CC(=O)N1CCN(C2CN3c4cc(N5CCC(c6c(C)cnn6C6CCOC6)C(C)C5)nc(C(F)F)c4CCCC23)CC1C] | 1-(4-(4-(difluoromethyl)-2-(3-methyl- 4-(4-methyl-1-(tetrahydro-3-furanyl)- 1H-pyrazol-5-yl)-1-piperidinyl)-5,6,7, 7a,8,9-hexahydroazeto[1,2-a]pyrido [3,4-f]azepin-8-yl)-2-methyl-1-piper- azinyl)-2-propen-1-one |
| 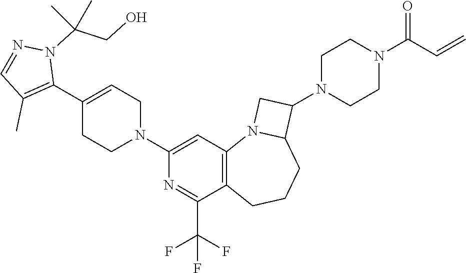 [SMILES: C=CC(=O)N1CCN(C2CN3c4cc(N5CC=C(c6c(C)cnn6C(C)(C)CO)CC5)nc(C(F)(F)F)c4CCCC23)CC1] | 1-(4-(4-(difluoromethyl)-2-(4-(1-(1- hydroxy-2-methyl-2-propanyl)-4- methyl-1H-pyrazol-5-yl)-3,6-dihydro- 1(2H)-pyridinyl)-5,6,7,7a,8,9-hexa- hydroazeto[1,2-a]pyrido[3,4-f]azepin- 8-yl)-1-piperazinyl)-2-propen-1-one |
| 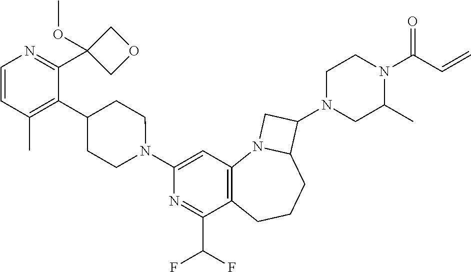 [SMILES: C=CC(=O)N1CCN(C2CN3c4cc(N5CCC(c6c(C)ccnc6C6(OC)COC6)CC5)nc(C(F)F)c4CCCC23)CC1C] | 1-(4-(4-(difluoromethyl)-2-(4-(2-(3- methoxy-3-oxetanyl)-4-methyl-3- pyridinyl)-1-piperidinyl)-5,6,7,7a,8,9- hexahydroazeto[1,2-a]pyrido[3,4-f] azepin-8-yl)-2-methyl-1-piperazinyl)- 2-propen-1-one |
| 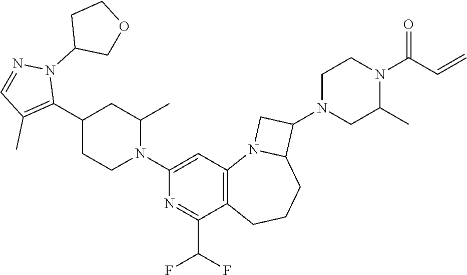 [SMILES: C=CC(=O)N1CCN(C2CN3c4cc(N5CCC(c6c(C)cnn6C6CCOC6)CC5C)nc(C(F)F)c4CCCC23)CC1C] | 1-(4-(4-(difluoromethyl)-2-(2-methyl- 4-(4-methyl-1-(tetrahydro-3-furanyl)- 1H-pyrazol-5-yl)-1-piperidinyl)-5,6,7, 7a,8,9-hexahydroazeto[1,2-a]pyrido [3,4-f]azepin-8-yl)-2-methyl-1-piper- azinyl)-2-propen-1-one |
| 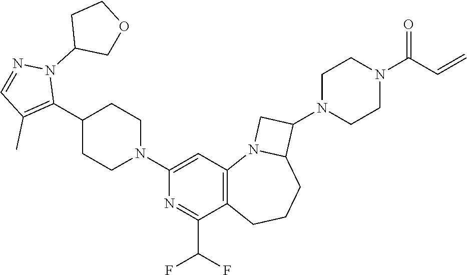 [SMILES: C=CC(=O)N1CCN(C2CN3c4cc(N5CCC(c6c(C)cnn6C6CCOC6)CC5)nc(C(F)F)c4CCCC23)CC1] | 1-(4-(4-(difluoromethyl)-2-(4-(4- methyl-1-(tetrahydro-3-furanyl)- 1H-pyrazol-5-yl)-1-piperidinyl)- 5,6,7,7a,8,9-hexahydroazeto[1,2-a] pyrido[3,4-f]azepin-8-yl)-1-piper- azin-yl)-2-propen-1-one |
| 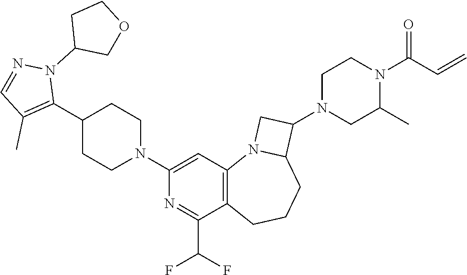 [SMILES: C=CC(=O)N1CCN(C2CN3c4cc(N5CCC(c6c(C)cnn6C6CCOC6)CC5)nc(C(F)F)c4CCCC23)CC1C] | 1-(4-(4-(difluoromethyl)-2-(4-(4- methyl-1-(tetrahydro-3-furanyl)-1H- pyrazol-5-yl)-1-piperidinyl)-5,6,7, 7a,8,9-hexahydroazeto[1,2-a]pyrido [3,4-f]azepin-8-yl)-2-methyl-1-piper- azinyl)-2-propen-1-one |
| 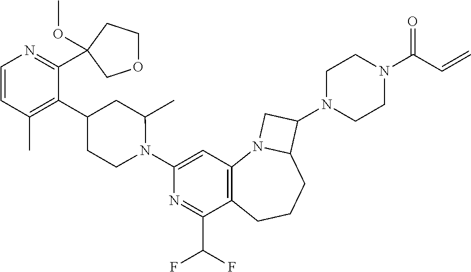 [SMILES: C=CC(=O)N1CCN(C2CN3c4cc(N5CCC(c6c(C)ccnc6C6(OC)CCOC6)CC5C)nc(C(F)F)c4CCCC23)CC1] | 1-(4-(4-(difluoromethyl)-2-(4-(2-(3- methoxytetrahydro-3-furanyl)-4- methyl-3-pyridinyl)-2-methyl-1- piperidinyl)-5,6,7,7a,8,9-hexahydro- azeto[1,2-a]pyrido[3,4-f]azepin-8- yl)-1-piperazinyl)-2-propen-1-one |
| 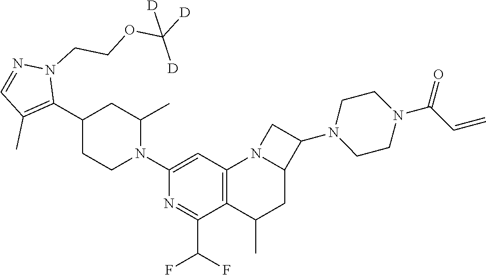 [SMILES: [2H]C([2H])([2H])OCCn1ncc(C)c1C1CCN(c2cc3c(c(C(F)F)n2)C(C)CC2C(N4CCN(C(=O)C=C)CC4)CN32)C(C)C1] | 1-(4-(4-(difluoromethyl)-2-(4-(1-(2- (methoxy-d3)ethyl)-4-methyl-1H- pyrazol-5-yl)-2-methylpiperidin-1- yl)-5-methyl-6,6a,7,8-tetrahydro-5H- azeto[1,2-a][1,6]naphthyridin-7-yl)- piperazin-1-yl)prop-2-en-1-one |
| 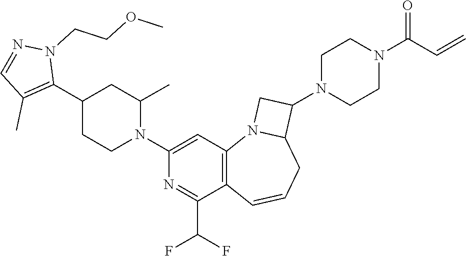 [SMILES: C=CC(=O)N1CCN(C2CN3c4cc(N5CCC(c6c(C)cnn6CCOC)CC5C)nc(C(F)F)c4C=CCC23)CC1] | 1-(4-(4-(difluoromethyl)-2-(4-(1-(2- methoxyethyl)-4-methyl-1H-pyrazol- 5-yl)-2-methyl-1-piperidinyl)-7,7a, 8,9-tetrahydroazeto[1,2-a]pyrido[3,4- f]azepin-8-yl)-1-piperazinyl)-2- propen-1-one |
| 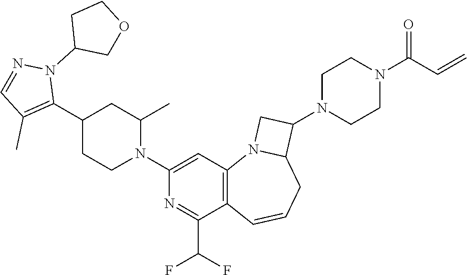 [SMILES: C=CC(=O)N1CCN(C2CN3c4cc(N5CCC(c6c(C)cnn6C6CCOC6)CC5C)nc(C(F)F)c4C=CCC23)CC1] | 1-(4-(4-(difluoromethyl)-2-(2-methyl- 4-(4-methyl-1-(tetrahydro-3-furanyl)- 1H-pyrazol-5-yl)-1-piperidinyl)-7,7a, 8,9-tetrahydroazeto[1,2-a]pyrido[3,4- f]azepin-8-yl)-1-piperazinyl)-2- propen-1-one |
| 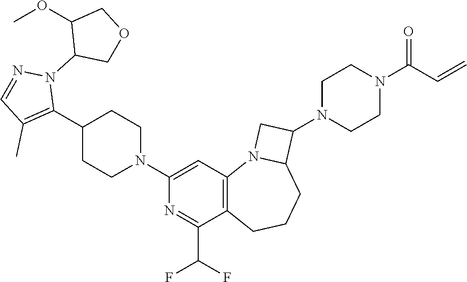 [SMILES: C=CC(=O)N1CCN(C2CN3c4cc(N5CCC(c6c(C)cnn6C6COCC6OC)CC5)nc(C(F)F)c4CCCC23)CC1] | 1-(4-(4-(difluoromethyl)-2-(4-(1-(4- methoxytetrahydro-3-furanyl)-4- methyl-1H-pyrazol-5-yl)-1-piperidin- yl)-5,6,7,7a,8,9-hexahydroazeto[1,2- a]pyrido[3,4-f]azepin-8-yl)-1-piper- azinyl)-2-propen-1-one |
| 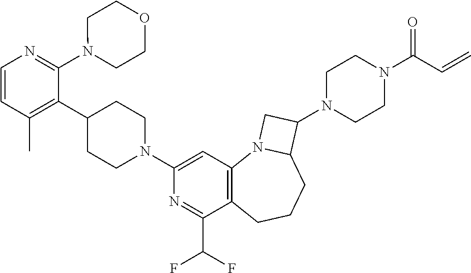 [SMILES: C=CC(=O)N1CCN(C2CN3c4cc(N5CCC(c6c(C)ccnc6N6CCOCC6)CC5)nc(C(F)F)c4CCCC23)CC1] | 1-(4-(4-(difluoromethyl)-2-(4-(4- methyl-2-(4-morpholinyl)-3-pyridin- yl)-1-piperidinyl)-5,6,7,7a,8,9-hexa- hydroazeto[1,2-a]pyrido[3,4-f]azepin- 8-yl)-1-piperazinyl)-2-propen-1-one |
| 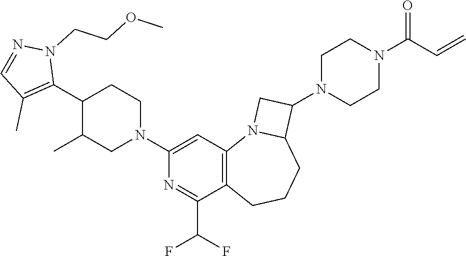 [SMILES: C=CC(=O)N1CCN(C2CN3c4cc(N5CCC(c6c(C)cnn6CCOC)C(C)C5)nc(C(F)F)c4CCCC23)CC1] | 1-(4-(4-(difluoromethyl)-2-(4-(1-(2- methoxyethyl)-4-methyl-1H-pyrazol- 5-yl)-3-methyl-1-piperidinyl)-5,6,7, 7a,8,9-hexahydroazeto[1,2-a]pyrido [3,4-f]azepin-8-yl)-1-piperazinyl)-2- propen-1-one |
| 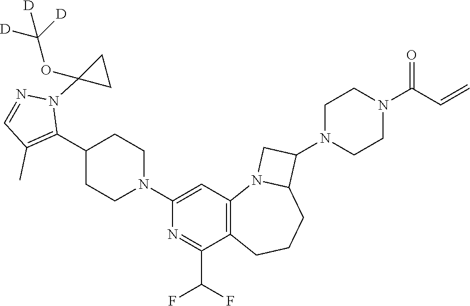 [SMILES: [2H]C([2H])([2H])OC1(n2ncc(C)c2C2CCN(c3cc4c(c(C(F)F)n3)CCCC3C(N5CCN(C(=O)C=C)CC5)CN43)CC2)CC1] | 1-(4-(4-(difluoromethyl)-2-(4-(1-(1- (methoxy-d3)cyclopropyl)-4-methyl- 1H-pyrazol-5-yl)piperidin-1-yl)- 5,6,7,7a,8,9-hexahydroazeto[1,2-a] pyrido[3,4-f]azepin-8-yl)piperazin-1- yl)prop-2-en-1-one |
| 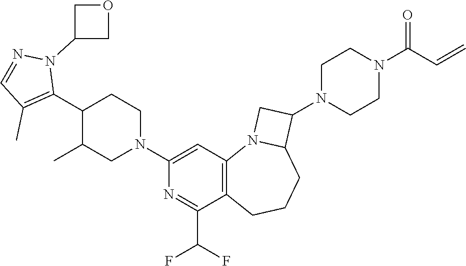 [SMILES: C=CC(=O)N1CCN(C2CN3c4cc(N5CCC(c6c(C)cnn6C6COC6)C(C)C5)nc(C(F)F)c4CCCC23)CC1] | 1-(4-(4-(difluoromethyl)-2-(3-meth- yl-4-(4-methyl-1-(3-oxetanyl)-1H- pyrazol-5-yl)-1-piperidinyl)-5,6,7, 7a,8,9-hexahydroazeto[1,2-a]pyrido [3,4-f]azepin-8-yl)-1-piperazinyl)-2- propen-1-one |
| 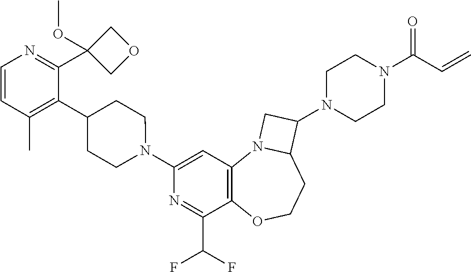 [SMILES: C=CC(=O)N1CCN(C2CN3c4cc(N5CCC(c6c(C)ccnc6C6(OC)COC6)CC5)nc(C(F)F)c4OCCC23)CC1] | 1-(4-(4-(difluoromethyl)-2-(4-(2-(3- methoxy-3-oxetanyl)-4-methyl-3- pyridinyl)-1-piperidinyl)-7,7a,8,9- tetrahydro-6H-azeto[1,2-d]pyrido [3,4-b][1,4]oxazepin-8-yl)-1-piper- azinyl)-2-propen-1-one |
| 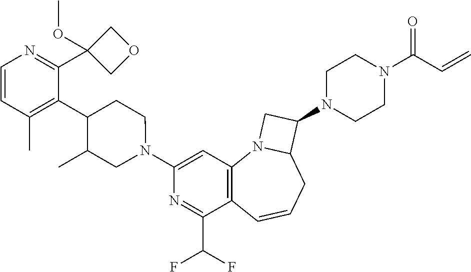 [SMILES: C=CC(=O)N1CCN([C@@H]2CN3c4cc(N5CCC(c6c(C)ccnc6C6(OC)COC6)C(C)C5)nc(C(F)F)c4C=CCC23)CC1] | 1-(4-(4-(difluoromethyl)-2-(4-(2-(3- methoxy-3-oxetanyl)-4-methyl-3- pyridinyl)-3-methyl-1-piperidinyl)- 7,7a,8,9-tetrahydroazeto[1,2-a]pyrido [3,4-f]azepin-8-yl)-1-piperazinyl)-2- propen-1-one |
| 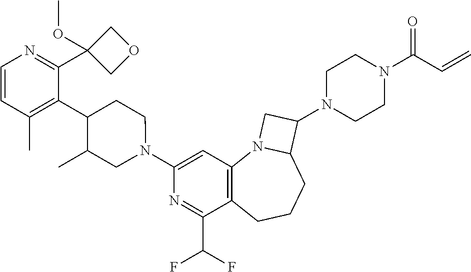 [SMILES: C=CC(=O)N1CCN(C2CN3c4cc(N5CCC(c6c(C)ccnc6C6(OC)COC6)C(C)C5)nc(C(F)F)c4CCCC23)CC1] | 1-(4-(4-(difluoromethyl)-2-(4-(2-(3- methoxy-3-oxetanyl)-4-methyl-3- pyridinyl)-3-methyl-1-piperidinyl)- 5,6,7,7a,8,9-hexahydroazeto[1,2-a] pyrido[3,4-f]azepin-8-yl)-1-piper- azinyl)-2-propen-1-one |
| 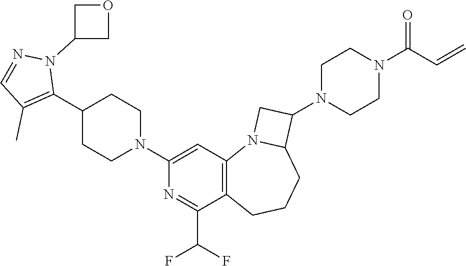 [SMILES: C=CC(=O)N1CCN(C2CN3c4cc(N5CCC(c6c(C)cnn6C6COC6)CC5)nc(C(F)F)c4CCCC23)CC1] | 1-(4-(4-(difluoromethyl)-2-(4-(4- methyl-1-(3-oxetanyl)-1H-pyrazol-5- yl)-1-piperidinyl])-5,6,7,7a,8,9-hexa- hydroazeto[1,2-a]pyrido[3,4-f]azepin- 8-yl)-1-piperazinyl)-2-propen-1-one |
| 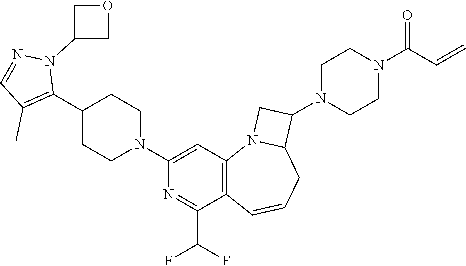 [SMILES: C=CC(=O)N1CCN(C2CN3c4cc(N5CCC(c6c(C)cnn6C6COC6)CC5)nc(C(F)F)c4C=CCC23)CC1] | 1-(4-(4-(difluoromethyl)-2-(3-meth- yl-4-(4-methyl-1-(3-oxetanyl)-1H- pyrazol-5-yl)-1-piperidinyl)-7,7a,8,9- tetrahydroazeto[1,2-a]pyrido[3,4-f] azepin-8-yl)-1-piperazinyl)-2-propen- 1-one |
| 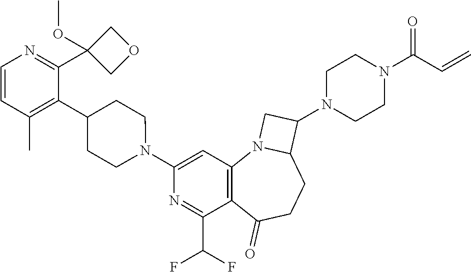 [SMILES: C=CC(=O)N1CCN(C2CN3c4cc(N5CCC(c6c(C)ccnc6C6(OC)COC6)CC5)nc(C(F)F)c4C(=O)CCC23)CC1] | 4-(difluoromethyl)-2-(4-(2-(3-meth- oxy-3-oxetanyl)-4-methyl-3-pyridin- yl])-1-piperidinyl)-8-(4-(2-propeno- yl)-1-piperazinyl)-7,7a,8,9-tetrahydro- azeto[1,2-a]pyrido[3,4-f]azepin- 5(6H)-one |
| 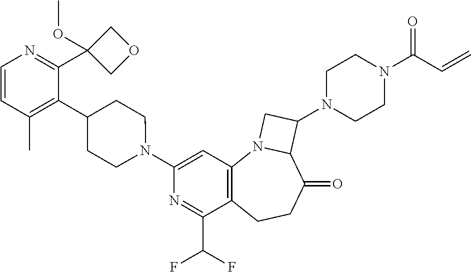 [SMILES: C=CC(=O)N1CCN(C2CN3c4cc(N5CCC(c6c(C)ccnc6C6(OC)COC6)CC5)nc(C(F)F)c4CCC(=O)C23)CC1] | 4-(difluoromethyl)-2-(4-(2-(3-meth- oxy-3-oxetanyl)-4-methyl-3-pyridin- yl)-1-piperidinyl)-8-(4-(2-propenoyl)- 1-piperazinyl)-5,6,8,9-tetrahydro- azeto[1,2-a]pyrido[3,4-f]azepin- 7(7aH)-one |
| 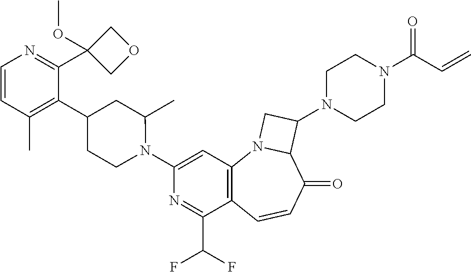 [SMILES: C=CC(=O)N1CCN(C2CN3c4cc(N5CCC(c6c(C)ccnc6C6(OC)COC6)CC5C)nc(C(F)F)c4C=CC(=O)C23)CC1] | 1-(4-(4-(difluoromethyl)-2-(4-(2-(3- methoxy-3-oxetanyl)-4-methyl-3- pyridinyl)-2-methyl-1-piperidinyl)- 7,7a,8,9-tetrahydroazeto[1,2-a]pyrido [3,4-f]azepin-8-yl)-1-piperazinyl)-2- propen-1-one |
| 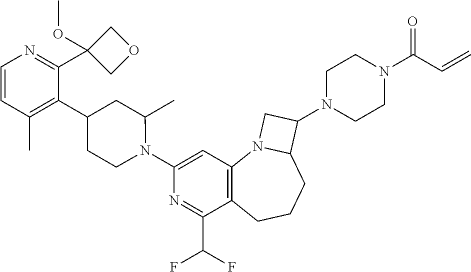 [SMILES: C=CC(=O)N1CCN(C2CN3c4cc(N5CCC(c6c(C)ccnc6C6(OC)COC6)CC5C)nc(C(F)F)c4CCCC23)CC1] | 1-(4-(4-(difluoromethyl)-2-(4-(2-(3- methoxy-3-oxetanyl)-4-methyl-3- pyridinyl)-2-methyl-1-piperidinyl)- 5,6,7,7a,8,9-hexahydroazeto[1,2-a] pyrido[3,4-f]azepin-8-yl)-1-piper- azinyl)-2-propen-1-one |
| 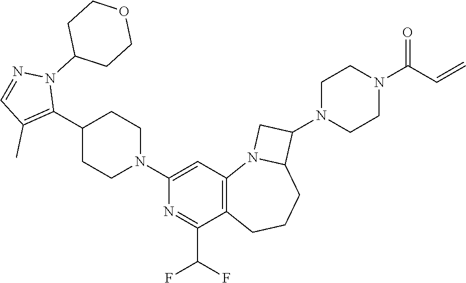 [SMILES: C=CC(=O)N1CCN(C2CN3c4cc(N5CCC(c6c(C)cnn6C6CCOCC6)CC5)nc(C(F)F)c4CCCC23)CC1] | 1-(4-(4-(difluoromethyl)-2-(4-(4- methyl-1-(tetrahydro-2H-pyran-4- yl)-1H-pyrazol-5-yl)-1-piperidinyl)- 5,6,7,7a,8,9-hexahydroazeto[1,2-a] pyrido[3,4-f]azepin-8-yl)-1-piper- azinyl)-2-propen-1-one |
| 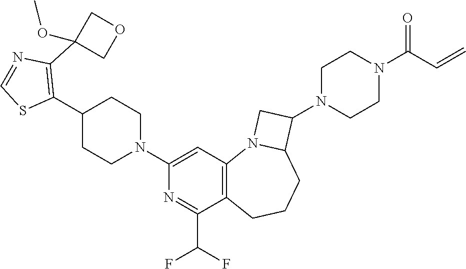 [SMILES: C=CC(=O)N1CCN(C2CN3c4cc(N5CCC(c6scnc6C6(OC)COC6)CC5)nc(C(F)F)c4CCCC23)CC1] | 1-(4-(4-(difluoromethyl)-2-(4-(4-(3- methoxy-3-oxetanyl)-1,3-thiazol-5- yl)-1-piperidinyl)-5,6,7,7a,8,9-hexa- hydroazeto[1,2-a]pyrido[3,4-f]azepin- 8-yl)-1-piperazinyl)-2-propen-1-one |
| 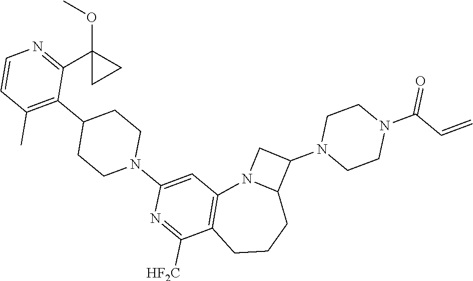 | 1-(4-(4-(difluoromethyl)-2-(4-(2-(1- methoxycyclopropyl)-4-methyl-3- pyridinyl)-1-piperidinyl)-5,6,7,7a,8,9- hexahydroazeto[1],2-a]pyrido[3,4-f] azepin-8-yl)-1-piperazinyl)-2-propen- 1-one |
| 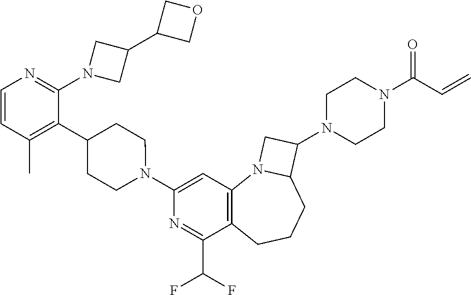 [SMILES: C=CC(=O)N1CCN(C2CN3c4cc(N5CCC(c6c(C)ccnc6N6CC(C7COC7)C6)CC5)nc(C(F)F)c4CCCC23)CC1] | 1-(4-(4-(difluoromethyl)-2-(4-(4- methyl-2-(3-(3-oxetanyl)-1-azetidin- yl)-3-pyridinyl)-1-piperidinyl)-5,6,7, 7a,8,9-hexahydroazeto[1,2-a]pyrido [3,4-f]azepin-8-yl)-1-piperazinyl)-2- propen-1-one |
| 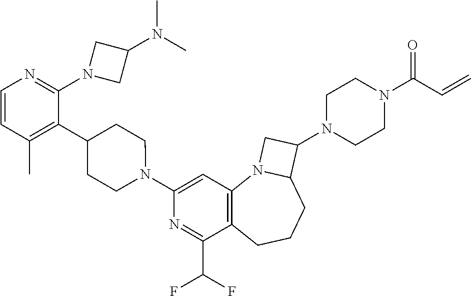 [SMILES: C=CC(=O)N1CCN(C2CN3c4cc(N5CCC(c6c(C)ccnc6N6CC(N(C)C)C6)CC5)nc(C(F)F)c4CCCC23)CC1] | 1-(4-(4-(difluoromethyl)-2-(4-(2-(3- (dimethylamino)-1-azetidinyl)-4- methyl-3-pyridinyl)-1-piperidinyl)- 5,6,7,7a,8,9-hexahydroazeto[1,2-a] pyrido[3,4-f]azepin-8-yl)-1-piperazin- yl)-2-propon-1-one |
| 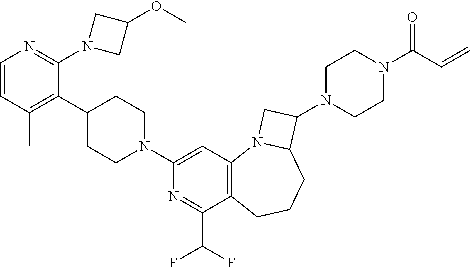 [SMILES: C=CC(=O)N1CCN(C2CN3c4cc(N5CCC(c6c(C)ccnc6N6CC(OC)C6)CC5)nc(C(F)F)c4CCCC23)CC1] | 1-(4-(4-(difluoromethyl)-2-(4-(2-(3- methoxy-1-azetidinyl)-4-methyl-3- pyridinyl)-1-piperidinyl)-5,6,7,7a,8,9- hexahydroazeto[1,2-a]pyrido[3,4-f] azepin-8-yl)-1-piperazinyl)-2-propen- 1-one |
| 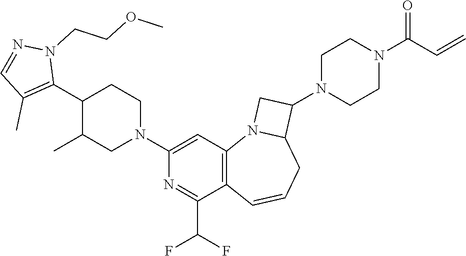 [SMILES: C=CC(=O)N1CCN(C2CN3c4cc(N5CCC(c6c(C)cnn6CCOC)C(C)C5)nc(C(F)F)c4C=CCC23)CC1] | 1-(4-(4-(difluoromethyl)-2-(4-(1-(2- methoxyethyl)-4-methyl-1H-pyrazol- 5-yl)-3-methyl-1-piperidinyl)-7,7a, 8,9-tetrahydroazeto[1,2-a]pyrido[3,4- flazepin-8-yl)-1-piperazinyl)-2- propen-1-one |
| 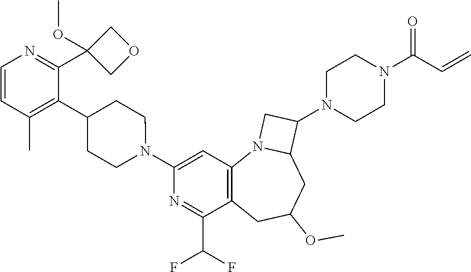 [SMILES: C=CC(=O)N1CCN(C2CN3c4cc(N5CCC(c6c(C)ccnc6C6(OC)COC6)CC5)nc(C(F)F)c4CC(OC)CC23)CC1] | 1-(4-(4-(difluoromethyl)-6-methoxy- 2-(4-(2-(3-methoxyoxetan-3-yl)-4- methylpyridin-3-yl)piperidin-1-yl)- 5,6,7,7a,8,9-hexahydroazeto[1,2-a] pyrido[3,4-f]azepin-8-yl)piperazin- 1-yl)prop-2-en-1-one |
| 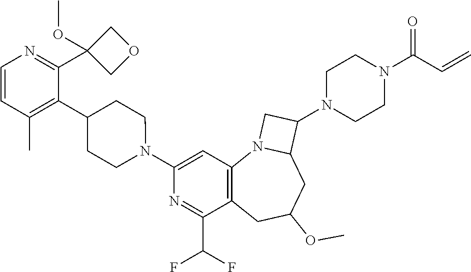 [SMILES: C=CC(=O)N1CCN(C2CN3c4cc(N5CCC(c6c(C)ccnc6C6(OC)COC6)CC5)nc(C(F)F)c4CC(OC)CC23)CC1] | 1-(4-(4-(difluoromethyl)-6-methoxy- 2-(4-(2-(3-methoxy-3-oxetanyl)-4- methyl-3-pyridinyl)-1-piperidinyl)- 5,6,7,7a,8,9-hexahydroazeto[1,2-a] pyrido[3,4-f]azepin-8-yl)-1-piperazin- yl)-2-propen-1-one |
| 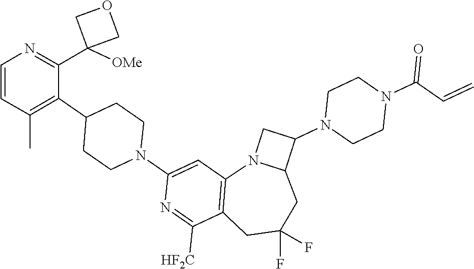 | 1-(4-(4-(difluoromethyl)-6,6-difluoro- 2-(4-(2-(3-methoxy-3-oxetanyl)-4- methyl-3-pyridinyl)-1-piperidinyl)- 5,6,7,7a,8,9-hexahydroazeto[1,2-a] pyrido[3,4-f]azepin-8-yl)-1-piperazin- yl)-2-propen-1-one |
| 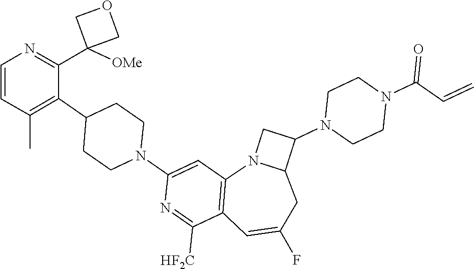 | 1-(4-(4-(difluoromethyl)-6-fluoro-2- (4-(2-(3-methoxy-3-oxetanyl)-4- methyl-3-pyridinyl)-1-piperidinyl)- 7,7a,8,9-tetrahydroazeto[1,2-a]pyrido [3,4-f]azepin-8-yl)-1-piperazinyl)-2- propen-1-one |
| 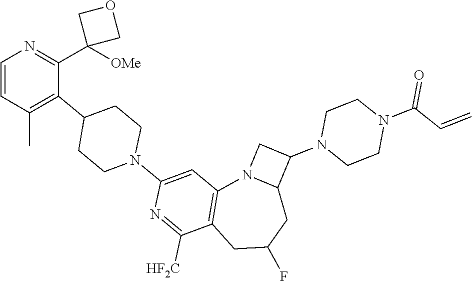 | 1-(4-(4-(difluoromethyl)-6-fluoro-2- (4-(2-(3-methoxy-3-oxetanyl)-4- methyl-3-pyridinyl)-1-piperidinyl)- 5,6,7,7a,8,9-hexahydroazeto[1,2-a] pyrido[3,4-f]azepin-8-yl)-1-piper- azinyl)-2-propen-1-one |
| 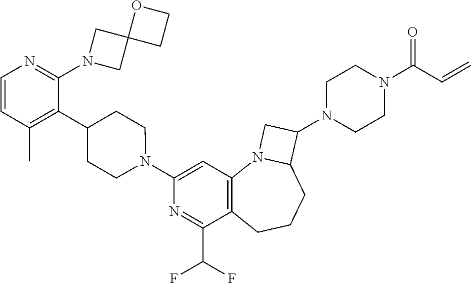 [SMILES: C=CC(=O)N1CCN(C2CN3c4cc(N5CCC(c6c(C)ccnc6N6CC7(CCO7)C6)CC5)nc(C(F)F)c4CCCC23)CC1] | 1-(4-(4-(difluoromethyl)-2-(4-(4- methyl-2-(1-oxa-6-azaspiro[3.3] heptan-6-yl)-3-pyridinyl)-1-piperidin- yl)-5,6,7,7a,8,9-hexahydroazeto[1,2- a]pyrido[3,4-f]azepin-8-yl)-1-piper- azinyl)-2-propen-1-one |
| 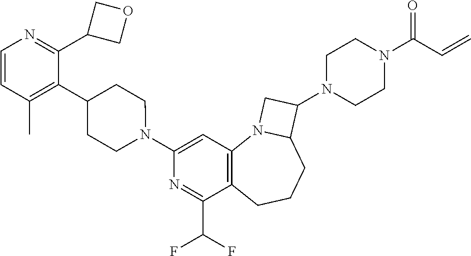 [SMILES: C=CC(=O)N1CCN(C2CN3c4cc(N5CCC(c6c(C)ccnc6C6COC6)CC5)nc(C(F)F)c4CCCC23)CC1] | 1-(4-(4-(difluoromethyl)-2-(4-(4- methyl-2-(3-oxetanyl)-3-pyridinyl)- 1-piperidinyl)-5,6,7,7a,8,9-hexahydro- azeto[1,2-a]pyrido[3,4-f]azepin-8-yl)- 1-piperazinyl)-2-propen-1-one |
| 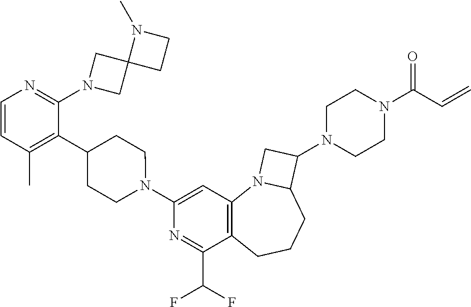 [SMILES: C=CC(=O)N1CCN(C2CN3c4cc(N5CCC(c6c(C)ccnc6N6CC7(CCN7C)C6)CC5)nc(C(F)F)c4CCCC23)CC1] | 1-(4-(4-(difluoromethyl)-2-(4-(4- methyl-2-(1-methyl-1,6-diazaspiro [3.3]heptan-6-yl)-3-pyridinyl)-1- piperidinyl)-5,6,7,7a,8,9-hexahydro- azeto[1,2-a]pyrido[3,4-f]azepin-8-yl)- 1-piperazinyl)-2-propen-1-one |
| 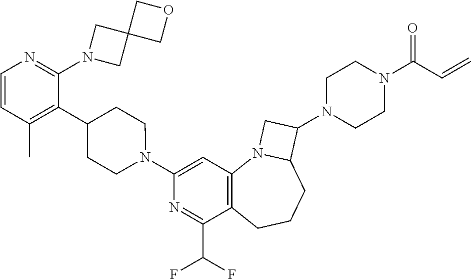 [SMILES: C=CC(=O)N1CCN(C2CN3c4cc(N5CCC(c6c(C)ccnc6N6CC7(COC7)C6)CC5)nc(C(F)F)c4CCCC23)CC1] | 1-(4-(4-(difluoromethyl)-2-(4-(4- methyl-2-(2-oxa-6-azaspiro[3.3] heptan-6-yl)-3-pyridinyl)-1-piperidin- yl)-5,6,7,7a,8,9-hexahydroazeto[1,2- a]pyrido[3,4-f]azepin-8-yl)-1-piper- azinyl)-2-propen-1-one |
| 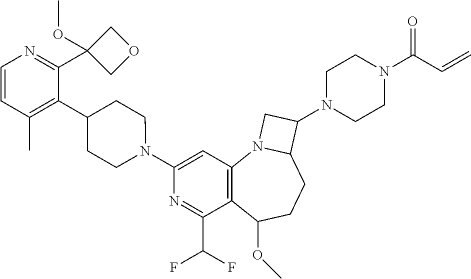 [SMILES: C=CC(=O)N1CCN(C2CN3c4cc(N5CCC(c6c(C)ccnc6C6(OC)COC6)CC5)nc(C(F)F)c4C(OC)CCC23)CC1] | 1-(4-(4-(difluoromethyl)-5-methoxy- 2-(4-(2-(3-methoxy-3-oxetanyl)-4- methyl-3-pyridinyl)-1-piperidinyl)- 5,6,7,7a,8,9-hexahydroazeto[1,2-a] pyrido[3,4-f]azepin-8-yl)-1-piperazin- yl)-2-propen-1-one |
| 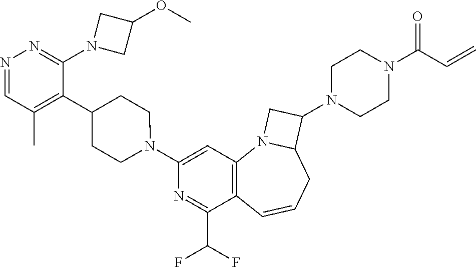 [SMILES: C=CC(=O)N1CCN(C2CN3c4cc(N5CCC(c6c(C)cnnc6N6CC(OC)C6)CC5)nc(C(F)F)c4C=CCC23)CC1] | 1-(4-((7aS,8R)-4-(difluoromethyl)-2- (4-(3-(3-methoxy-1-azetidinyl)-5- methyl-4-pyridazinyl)-1-piperidinyl)- 7,7a,8,9-tetrahydroazeto[1,2-a]pyrido [3,4-f]azepin-8-yl)-1-piperazinyl)-2- propen-1-one |
| 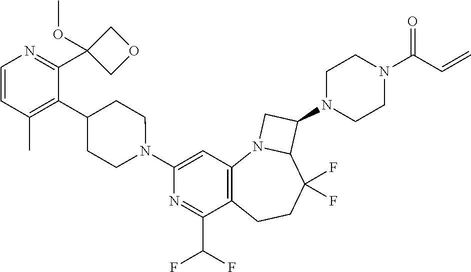 [SMILES: C=CC(=O)N1CCN([C@@H]2CN3c4cc(N5CCC(c6c(C)ccnc6C6(OC)COC6)CC5)nc(C(F)F)c4CCC(F)(F)C23)CC1] | 1-(4-(4-(difluoromethyl)-7,7-difluoro- 2-(4-(2-(3-methoxy-3-oxetanyl)-4- methyl-3-pyridinyl)-1-piperidinyl)- 5,6,7,7a,8,9-hexahydroazeto[1,2-a] pyrido[3,4-f]azepin-8-yl)-1-piper- azinyl)-2-propen-1-one |
| 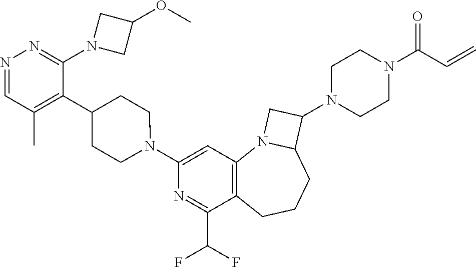 [SMILES: C=CC(=O)N1CCN(C2CN3c4cc(N5CCC(c6c(C)cnnc6N6CC(OC)C6)CC5)nc(C(F)F)c4CCCC23)CC1] | 1-(4-(4-(difluoromethyl)-2-(4-(3-(3- methoxy-1-azetidinyl)-5-methyl-4- pyridazinyl)-1-piperidinyl)-5,6,7,7a, 8,9-hexahydroazeto[1,2-a]pyrido [3,4-f]azepin-8-yl)-1-piperazinyl)-2- propen-1-one |
| 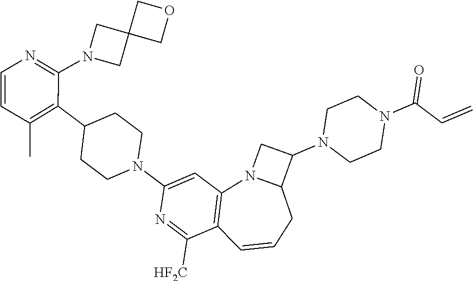 | 1-(4-(4-(Difluoromethyl)-2-(4-(5- methyl-3-(2-oxa-6-azaspiro[3.3] heptan-6-yl)-4-pyridazinyl)-1-piper- idinyl)-7,7a,8,9-tetrahydroazeto[1,2- a]pyrido[3,4-f]azepin-8-yl)-1-piper- azinyl)-2-propen-1-one |
| 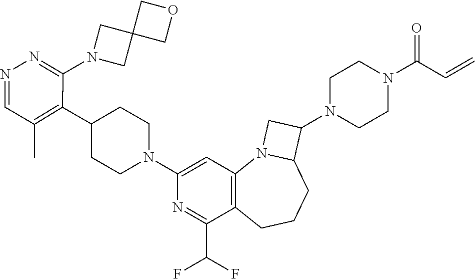 [SMILES: C=CC(=O)N1CCN(C2CN3c4cc(N5CCC(c6c(C)cnnc6N6CC7(COC7)C6)CC5)nc(C(F)F)c4CCCC23)CC1] | 1-(4-(4-(difluoromethyl)-2-(4-(5- methyl-3-(2-oxa-6-azaspiro[3.3] heptan-6-yl)-4-pyridazinyl)-1-piper- idinyl)-5,6,7,7a,8,9-hexahydroazeto [1,2-a]pyrido[3,4-f]azepin-8-yl)-1- piperazinyl)-2-propen-1-one |
| 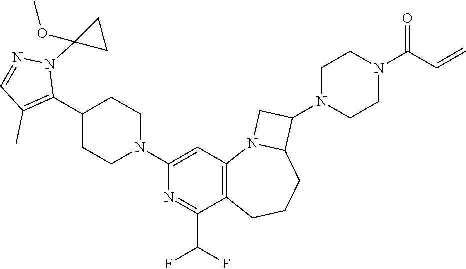 [SMILES: C=CC(=O)N1CCN(C2CN3c4cc(N5CCC(c6c(C)cnn6C6(OC)CC6)CC5)nc(C(F)F)c4CCCC23)CC1] | 1-(4-(4-(difluoromethyl)-2-(4-(1-(1- methoxycyclopropyl)-4-methyl-1H- pyrazol-5-yl)-1-piperidinyl)-5,6,7,7a, 8,9-hexahydroazeto[1,2-a]pyrido [3,4-f]azepin-8-yl)-1-piperazinyl)-2- propen-1-one |
| 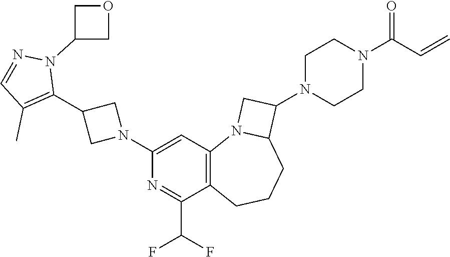 [SMILES: C=CC(=O)N1CCN(C2CN3c4cc(N5CC(c6c(C)cnn6C6COC6)C5)nc(C(F)F)c4CCCC23)CC1] | 1-(4-(4-(difluoromethyl)-2-(3-(4- methyl-1-(3-oxetanyl)-1H-pyrazol-5- yl)-1-azetidinyl)-5,6,7,7a,8,9-hexa- hydroazeto[1,2-a]pyrido[3,4-f]azepin- 8-yl)-1-piperazinyl)-2-propen-1-one |
| 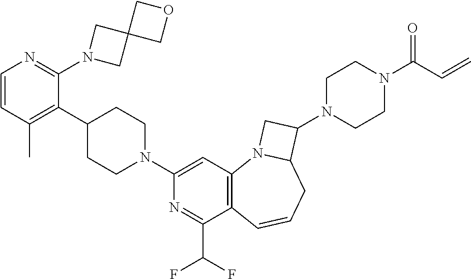 [SMILES: C=CC(=O)N1CCN(C2CN3c4cc(N5CCC(c6c(C)ccnc6N6CC7(COC7)C6)CC5)nc(C(F)F)c4C=CCC23)CC1] | 1-(4-(4-(difluoromethyl)-2-(4-(4- methyl-2-(2-oxa-6-azaspiro[3.3] heptan-6-yl)-3-pyridinyl)-1-piper- idinyl)-7,7a,8,9-tetrahydroazeto[1,2- a]pyrido[3,4-f]azepin-8-yl)-1-piper- azinyl)-2-propen-1-one |
| 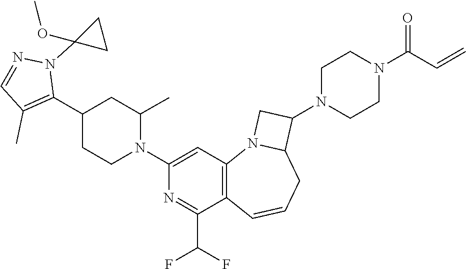 [SMILES: C=CC(=O)N1CCN(C2CN3c4cc(N5CCC(c6c(C)cnn6C6(OC)CC6)CC5C)nc(C(F)F)c4C=CCC23)CC1] | 1-(4-(4-(difluoromethyl)-2-(4-(1-(1- methoxycyclopropyl)-4-methyl-1H- pyrazol-5-yl)-2-methyl-1-piperidin- yl)-7,7a,8,9-tetrahydroazeto[1,2-a] pyrido[3,4-f]azepin-8-yl)-1-piper- azinyl)-2-propen-1-one |
| 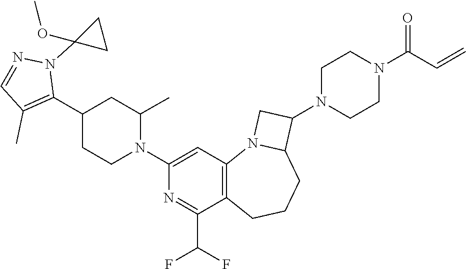 [SMILES: C=CC(=O)N1CCN(C2CN3c4cc(N5CCC(c6c(C)cnn6C6(OC)CC6)CC5C)nc(C(F)F)c4CCCC23)CC1] | 1-(4-(4-(difluoromethyl)-2-(4-(1-(1- methoxycyclopropyl)-4-methyl-1H- pyrazol-5-yl)-2-methyl-1-piperidin- yl)-5,6,7,7a,8,9-hexahydroazeto[1,2- a]pyrido[3,4-f]azepin-8-yl)-1-piper- azinyl)-2-propen-1-one |
| 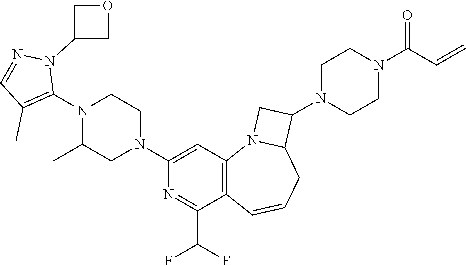 [SMILES: C=CC(=O)N1CCN(C2CN3c4cc(N5CCN(c6c(C)cnn6C6COC6)C(C)C5)nc(C(F)F)c4C=CCC23)CC1] | 1-(4-(4-(difluoromethyl)-2-(3-methyl- 4-(4-methyl-1-(3-oxetanyl)-1H- pyrazol-5-yl)-1-piperazinyl)-7,7a,8,9- tetrahydroazeto[1,2-a]pyrido[3,4-f] azepin-8-yl)-1-piperazinyl)-2-propen- 1-one |
| 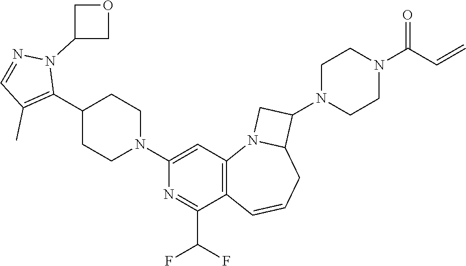 [SMILES: C=CC(=O)N1CCN(C2CN3c4cc(N5CCC(c6c(C)cnn6C6COC6)CC5)nc(C(F)F)c4C=CCC23)CC1] | 1-(4-(4-(difluoromethyl)-2-(4-(4- methyl-1-(3-oxetanyl)-1H-pyrazol-5- yl)-1-piperidinyl)-7,7a,8,9-tetrahydro- azeto[1,2-a]pyrido[3,4-f]azepin-8-yl)- 1-piperazinyl)-2-propen-1-one |
| 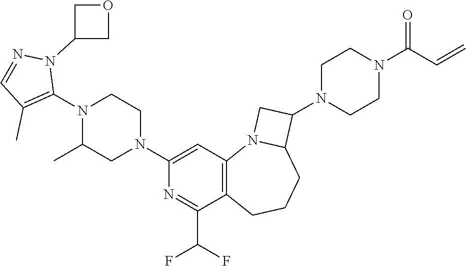 [SMILES: C=CC(=O)N1CCN(C2CN3c4cc(N5CCN(c6c(C)cnn6C6COC6)C(C)C5)nc(C(F)F)c4CCCC23)CC1] | 1-(4-(4-(difluoromethyl)-2-(3-meth- yl-4-(4-methyl-1-(3-oxetanyl)-1H- pyrazol-5-yl)-1-piperazinyl)-5,6,7,7a, 8,9-hexahydroazeto[1,2-a]pyrido [3,4-f]azepin-8-yl)-1-piperazinyl)-2- propen-1-one |
| 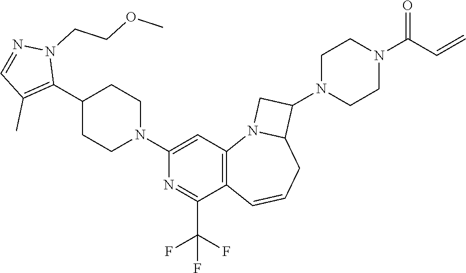 [SMILES: C=CC(=O)N1CCN(C2CN3c4cc(N5CCC(c6c(C)cnn6CCOC)CC5)nc(C(F)(F)F)c4C=CCC23)CC1] | 1-(4-(2-(4-(1-(2-Methoxyethyl)-4- methyl-1H-pyrazol-5-yl)-1-piperidin- yl)-4-(trifluoromethyl)-7,7a,8,9-tetra- hydroazeto[1,2-a]pyrido[3,4-f]azepin- 8-yl)-1-piperazinyl)-2-propen-1-one |
| 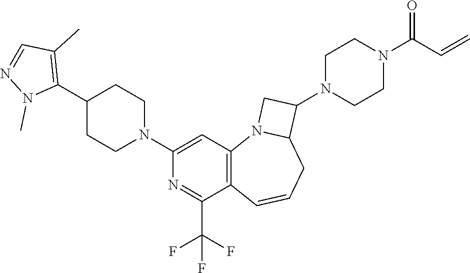 [SMILES: C=CC(=O)N1CCN(C2CN3c4cc(N5CCC(c6c(C)cnn6C)CC5)nc(C(F)(F)F)c4C=CCC23)CC1] | 1-(4-(2-(4-(1,4-dimethyl-1H-pyrazol- 5-yl)-1-piperidinyl)-4-(trifluorometh- yl)-7,7a,8,9-tetrahydroazeto[1,2-a] pyrido[3,4-f]azepin-8-yl)-1-piperazin- yl)-2-propen-1-one |
| 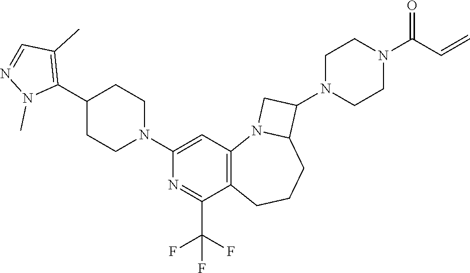 [SMILES: C=CC(=O)N1CCN(C2CN3c4cc(N5CCC(c6c(C)cnn6C)CC5)nc(C(F)(F)F)c4CCCC23)CC1] | 1-(4-(2-(4-(1,4-dimethyl-1H-pyrazol- 5-yl)-1-piperidinyl)-4-(trifluorometh- yl)-5,6,7,7a,8,9-hexahydroazeto[1,2- a]pyrido[3,4-f]azepin-8-yl)-1-piper- azinyl)-2-propen-1-one |
| 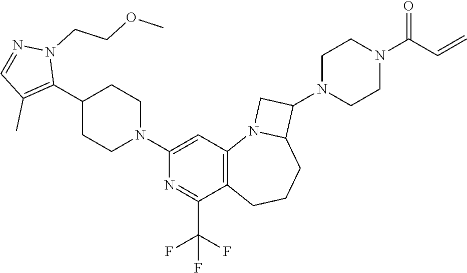 [SMILES: C=CC(=O)N1CCN(C2CN3c4cc(N5CCC(c6c(C)cnn6CCOC)CC5)nc(C(F)(F)F)c4CCCC23)CC1] | 1-(4-(2-(4-(1-(2-methoxyethyl)-4- methyl-1H-pyrazol-5-yl)-1-piperidin- yl)-4-(trifluoromethyl)-5,6,7,7a,8,9- hexahydroazeto[1,2-a]pyrido[3,4-f] azepin-8-yl)-1-piperazinyl)-2-propen- 1-one |
| 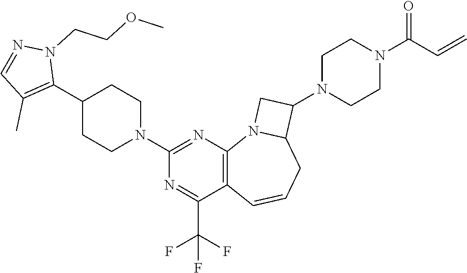 [SMILES: C=CC(=O)N1CCN(C2CN3c4nc(N5CCC(c6c(C)cnn6CCOC)CC5)nc(C(F)(F)F)c4C=CCC23)CC1] | 1-(4-(2-(4-(1-(2-methoxyethyl)-4- methyl-1H-pyrazol-5-yl)-1-piperidin- yl)-4-(trifluoromethyl)-7,7a,8,9-tetra- hydroazeto[1,2-a]pyrimido[5,4-f] azepin-8-yl)-1-piperazinyl)-2-propen- 1-one |
| 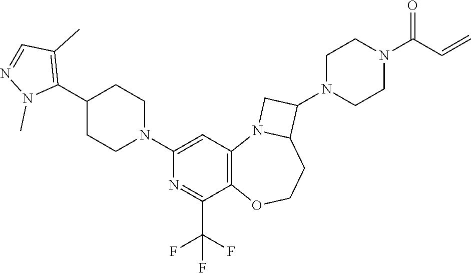 [SMILES: C=CC(=O)N1CCN(C2CN3c4cc(N5CCC(c6c(C)cnn6C)CC5)nc(C(F)(F)F)c4OCCC23)CC1] | 1-(4-(2-(4-(1,4-dimethyl-1H-pyrazol- 5-yl)-1-piperidinyl)-4-(trifluorometh- yl)-7,7a,8,9-tetrahydro-6H-azeto[1,2- d]pyrido[3,4-b][1,4]oxazepin-8-yl)-1- piperazinyl)-2-propen-1-one |
| 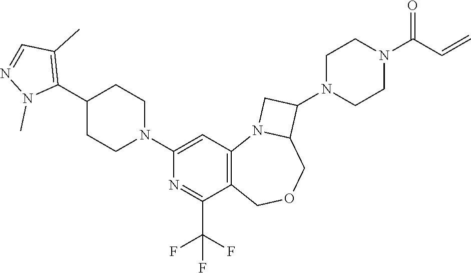 [SMILES: C=CC(=O)N1CCN(C2CN3c4cc(N5CCC(c6c(C)cnn6C)CC5)nc(C(F)(F)F)c4COCC23)CC1] | 1-(4-(2-(4-(1,4-dimethyl-1H-pyrazol- 5-yl)piperidin-1-yl)-4-(trifluorometh- yl)-7,7a,8,9-tetrahydro-5H-azeto[2,1- c]pyrido[4,3-e][1,4]oxazepin-8-yl)- piperazin-1-yl)prop-2-en-1-one |
| 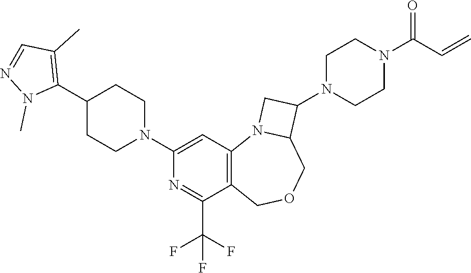 [SMILES: C=CC(=O)N1CCN(C2CN3c4cc(N5CCC(c6c(C)cnn6C)CC5)nc(C(F)(F)F)c4COCC23)CC1] | 1-(4-(2-(4-(1,4-dimethyl-1H-pyrazol- 5-yl)-1-piperidinyl)-4-(trifluorometh- yl)-7,7a,8,9-tetrahydro-5H-azeto[2,1- c]pyrido[4,3-e][1,4]oxazepin-8-yl)-1- piperazinyl)-2-propen-1-one |
| 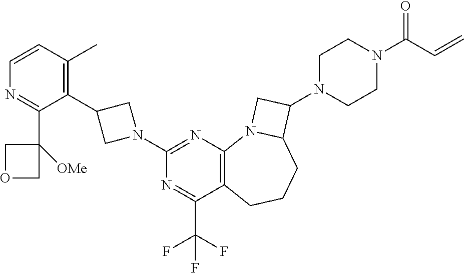 [SMILES: C=CC(=O)N1CCN(C2CN3c4nc(N5CC(c6c(C)ccnc6C6(OC)COC6)C5)nc(C(F)(F)F)c4CCCC23)CC1] | 1-(4-(2-(3-(2-(3-methoxyoxetan-3- yl)-4-methylpyridin-3-yl)azetidin-1]- yl)-4-(trifluoromethyl)-5,6,7,7a,8,9- hexahydroazeto[1,2-a]pyrimido[5,4- f]azepin-8-yl)piperazin-1-yl)prop-2- en-1-one |
| 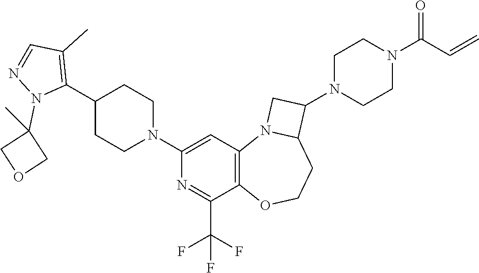 [SMILES: C=CC(=O)N1CCN(C2CN3c4cc(N5CCC(c6c(C)cnn6C6(C)COC6)CC5)nc(C(F)(F)F)c4OCCC23)CC1] | 1-(4-(2-(4-(4-methyl-1-(3-methyl- oxetan-3-yl)-1H-pyrazol-5-yl)piper- idin-1-yl)-4-(trifluoromethyl)-7,7a, 8,9-tetrahydro-6H-azeto[1,2-d]pyrido [3,4-b][1,4]oxazepin-8-yl)-piperazin- 1-yl)prop-2-en-1-one |
| 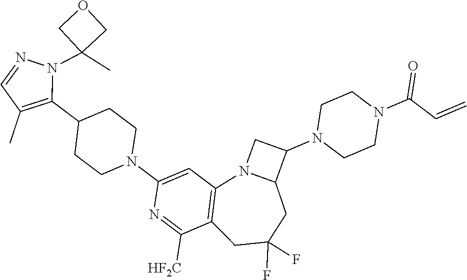 | 1-(4-(4-(difluoromethyl)-6,6-difluoro- 2-(4-(4-methyl-1-(3-methyloxetan-3- yl)-1H-pyrazol-5-yl)piperidin-1-yl)- 5,6,7,7a,8,9-hexahydroazeto[1,2-a] pyrido[3,4-f]azepin-8-yl)piperazin-1- yl)prop-2-en-1-one |
| 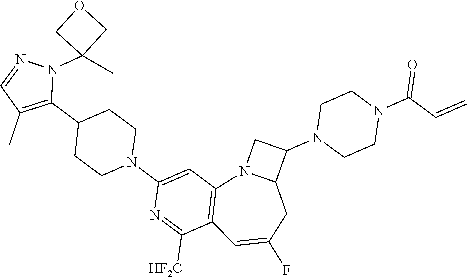 | 1-(4-(4-(difluoromethyl)-6-fluoro-2- (4-(4-methyl-1-(3-methyloxetan-3- yl)-1H-pyrazol-5-yl)piperidin-1-yl)- 7,7a,8,9-tetrahydroazeto[1,2-a]pyrido [3,4-f]azepin-8-yl)piperazin-1-yl)- prop-2-en-1-one |
| 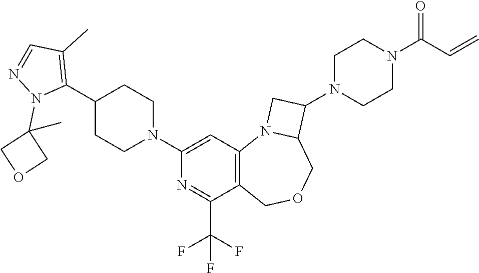 [SMILES: C=CC(=O)N1CCN(C2CN3c4cc(N5CCC(c6c(C)cnn6C6(C)COC6)CC5)nc(C(F)(F)F)c4COCC23)CC1] | 1 1-(4-(2-(4-(4-methyl-1-(3-methyl- oxetan-3-yl)-1H-pyrazol-5-yl)piper- idin-1-yl)-4-(trifluoromethyl)-7,7a, 8,9-tetrahydro-5H-azeto[2,1-c]pyrido [4,3-c][1,4]oxazepin-8-yl)-piperazin- 1-yl)prop-2-en-1-one |

In some cases, Formula (II) has a structure of Formula (IIB). Contemplated compounds of Formula (IIB) include, for example,

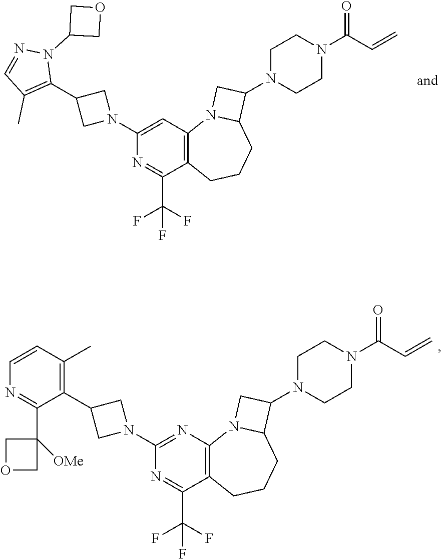 [SMILES: C=CC(=O)N1CCN(C2CN3c4cc(N5CC(c6c(C)cnn6C6COC6)C5)nc(C(F)(F)F)c4CCCC23)CC1 ; C=CC(=O)N1CCN(C2CN3c4nc(N5CC(c6c(C)ccnc6C6(OC)COC6)C5)nc(C(F)(F)F)c4CCCC23)CC1]

and pharmaceutically acceptable salts thereof.

In some cases, Formula (II) has a structure of Formula (HID). Contemplated compounds of Formula (IID) wherein Y is CH include, for example:

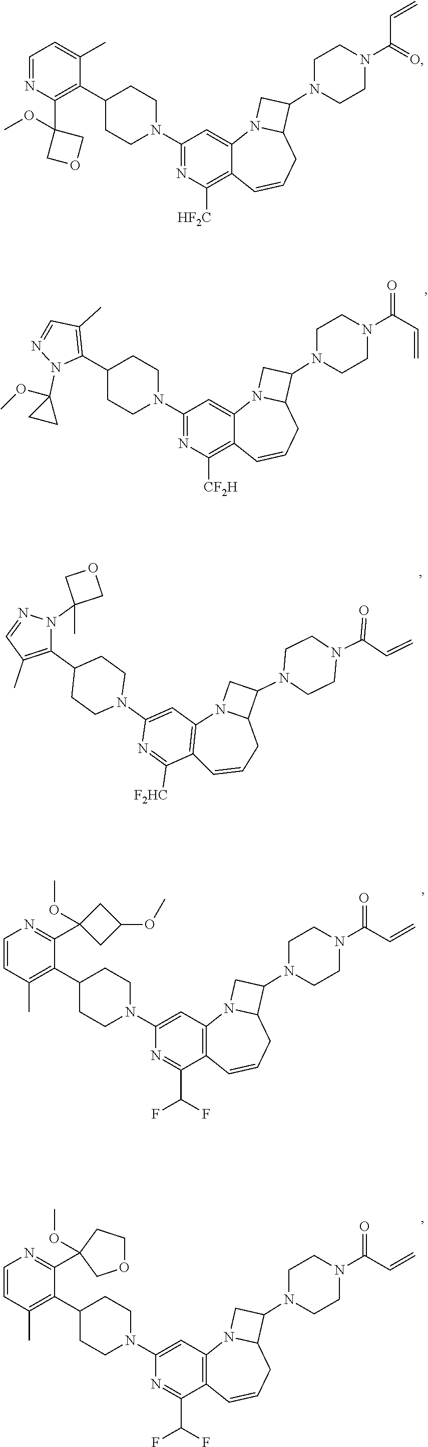

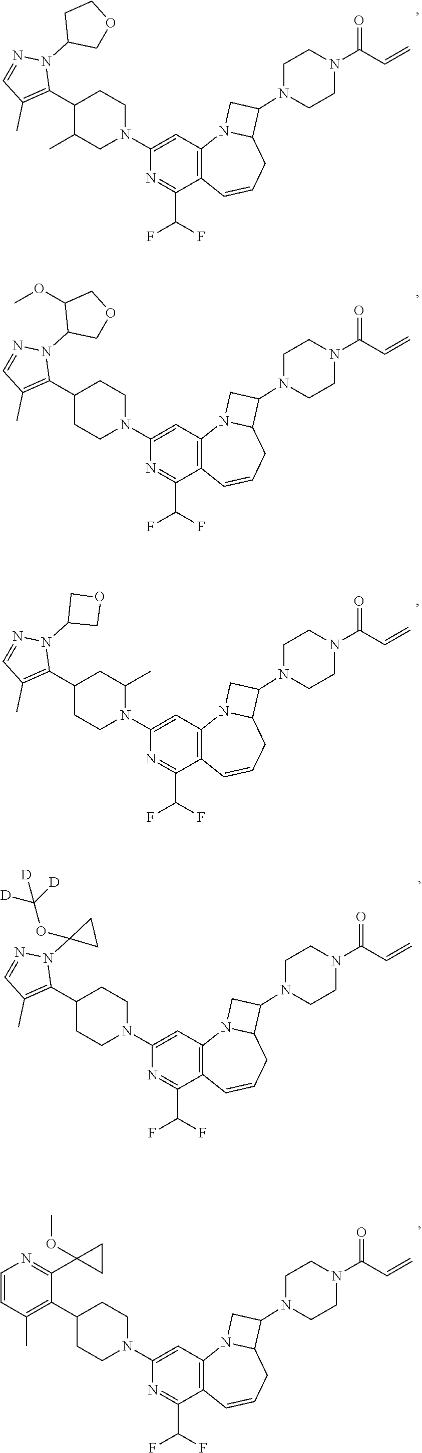 [SMILES: C=CC(=O)N1CCN(C2CN3c4cc(N5CCC(c6c(C)cnn6C6CCOC6)C(C)C5)nc(C(F)F)c4C=CCC23)CC1 ; C=CC(=O)N1CCN(C2CN3c4cc(N5CCC(c6c(C)cnn6C6COCC6OC)CC5)nc(C(F)F)c4C=CCC23)CC1 ; C=CC(=O)N1CCN(C2CN3c4cc(N5CCC(c6c(C)cnn6C6COC6)CC5C)nc(C(F)F)c4C=CCC23)CC1 ; [2H]C([2H])([2H])OC1(n2ncc(C)c2C2CCN(c3cc4c(c(C(F)F)n3)C=CCC3C(N5CCN(C(=O)C=C)CC5)CN43)CC2)CC1 ; C=CC(=O)N1CCN(C2CN3c4cc(N5CCC(c6c(C)ccnc6C6(OC)CC6)CC5)nc(C(F)F)c4C=CCC23)CC1]

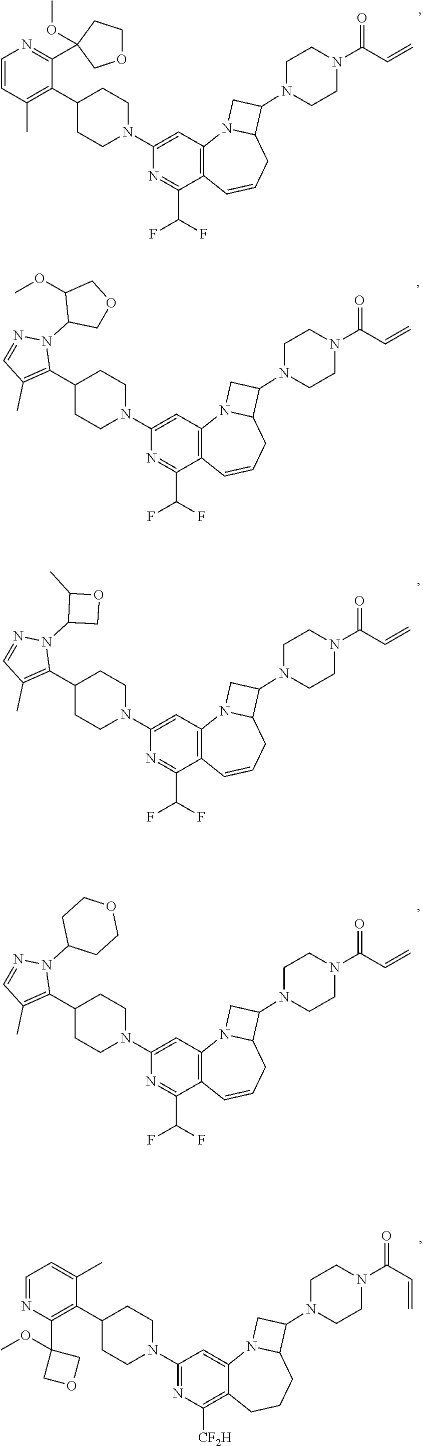

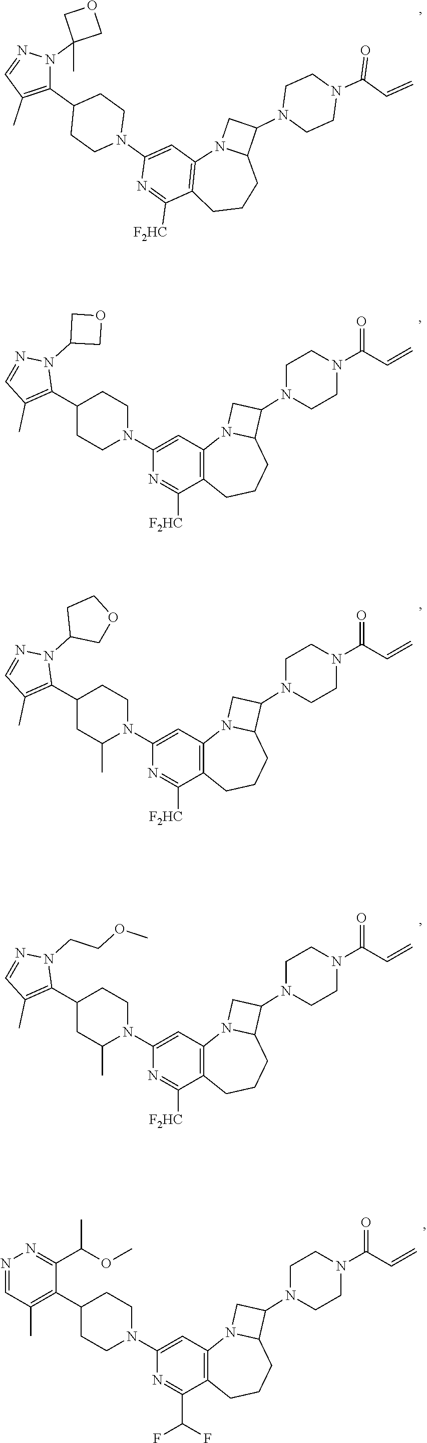 [SMILES: C=CC(=O)N1CCN(C2CN3c4cc(N5CCC(c6c(C)cnn6C6(C)COC6)CC5)nc(C(F)F)c4CCCC23)CC1 ; C=CC(=O)N1CCN(C2CN3c4cc(N5CCC(c6c(C)cnn6C6COC6)CC5)nc(C(F)F)c4CCCC23)CC1 ; C=CC(=O)N1CCN(C2CN3c4cc(N5CCC(c6c(C)cnn6C6CCOC6)CC5C)nc(C(F)F)c4CCCC23)CC1 ; C=CC(=O)N1CCN(C2CN3c4cc(N5CCC(c6c(C)cnn6CCOC)CC5C)nc(C(F)F)c4CCCC23)CC1 ; C=CC(=O)N1CCN(C2CN3c4cc(N5CCC(c6c(C)cnnc6C(C)OC)CC5)nc(C(F)F)c4CCCC23)CC1]

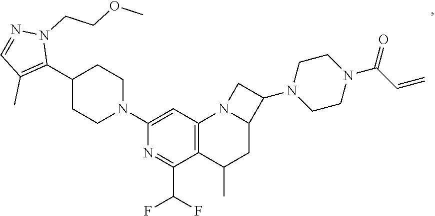 [SMILES: C=CC(=O)N1CCN(C2CN3c4cc(N5CCC(c6c(C)cnn6CCOC)CC5)nc(C(F)F)c4C(C)CC23)CC1]

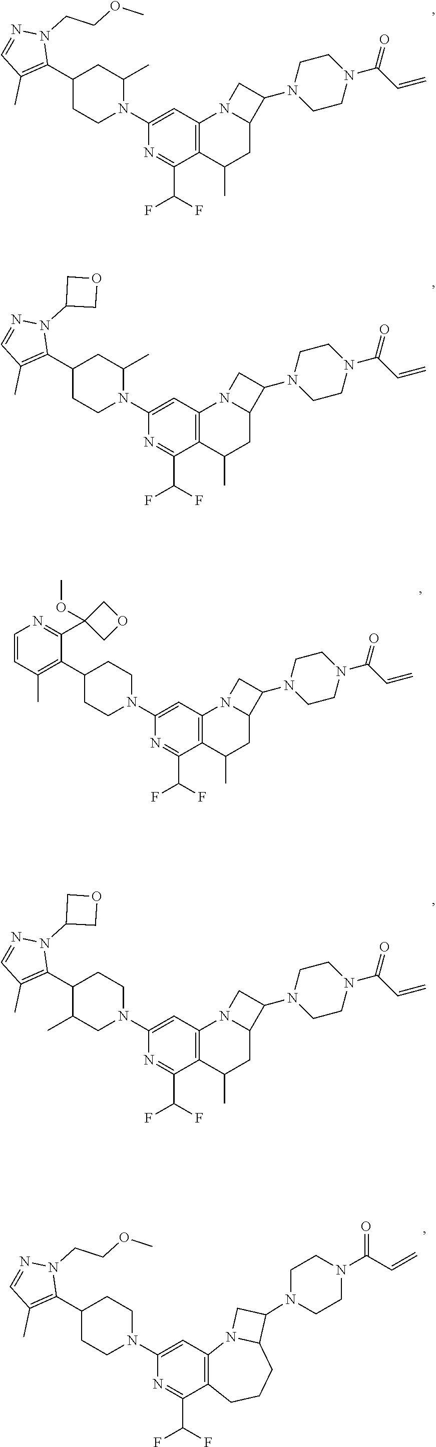 [SMILES: C=CC(=O)N1CCN(C2CN3c4cc(N5CCC(c6c(C)cnn6CCOC)CC5C)nc(C(F)F)c4C(C)CC23)CC1 ; C=CC(=O)N1CCN(C2CN3c4cc(N5CCC(c6c(C)cnn6C6COC6)CC5C)nc(C(F)F)c4C(C)CC23)CC1 ; C=CC(=O)N1CCN(C2CN3c4cc(N5CCC(c6c(C)ccnc6C6(OC)COC6)CC5)nc(C(F)F)c4C(C)CC23)CC1 ; C=CC(=O)N1CCN(C2CN3c4cc(N5CCC(c6c(C)cnn6C6COC6)C(C)C5)nc(C(F)F)c4C(C)CC23)CC1 ; C=CC(=O)N1CCN(C2CN3c4cc(N5CCC(c6c(C)cnn6CCOC)CC5)nc(C(F)F)c4CCCC23)CC1]

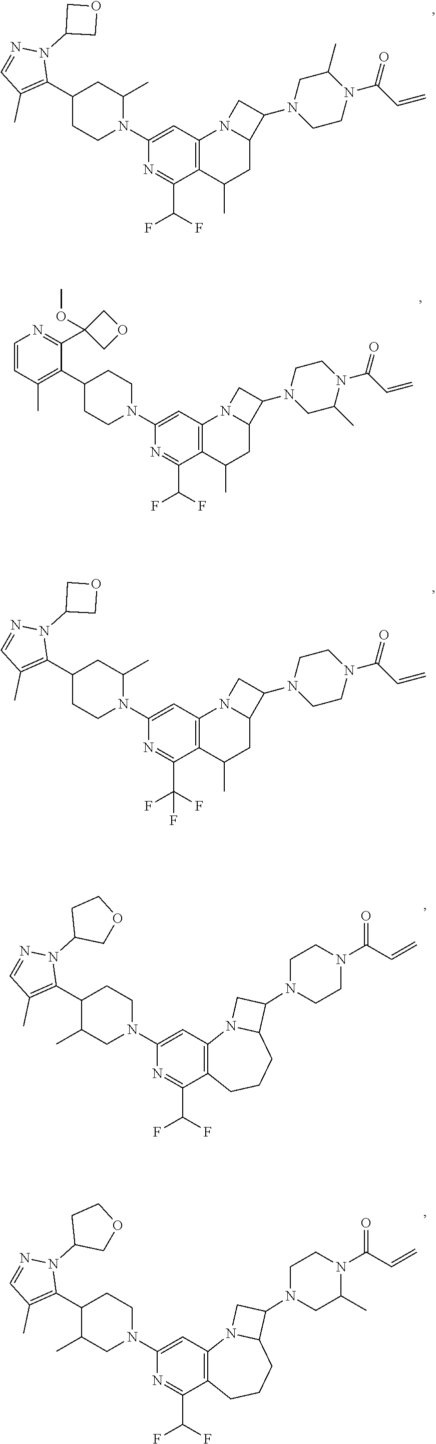 [SMILES: C=CC(=O)N1CCN(C2CN3c4cc(N5CCC(c6c(C)cnn6C6COC6)CC5C)nc(C(F)F)c4C(C)CC23)CC1C ; C=CC(=O)N1CCN(C2CN3c4cc(N5CCC(c6c(C)ccnc6C6(OC)COC6)CC5)nc(C(F)F)c4C(C)CC23)CC1C ; C=CC(=O)N1CCN(C2CN3c4cc(N5CCC(c6c(C)cnn6C6COC6)CC5C)nc(C(F)(F)F)c4C(C)CC23)CC1 ; C=CC(=O)N1CCN(C2CN3c4cc(N5CCC(c6c(C)cnn6C6CCOC6)C(C)C5)nc(C(F)F)c4CCCC23)CC1 ; C=CC(=O)N1CCN(C2CN3c4cc(N5CCC(c6c(C)cnn6C6CCOC6)C(C)C5)nc(C(F)F)c4CCCC23)CC1C]

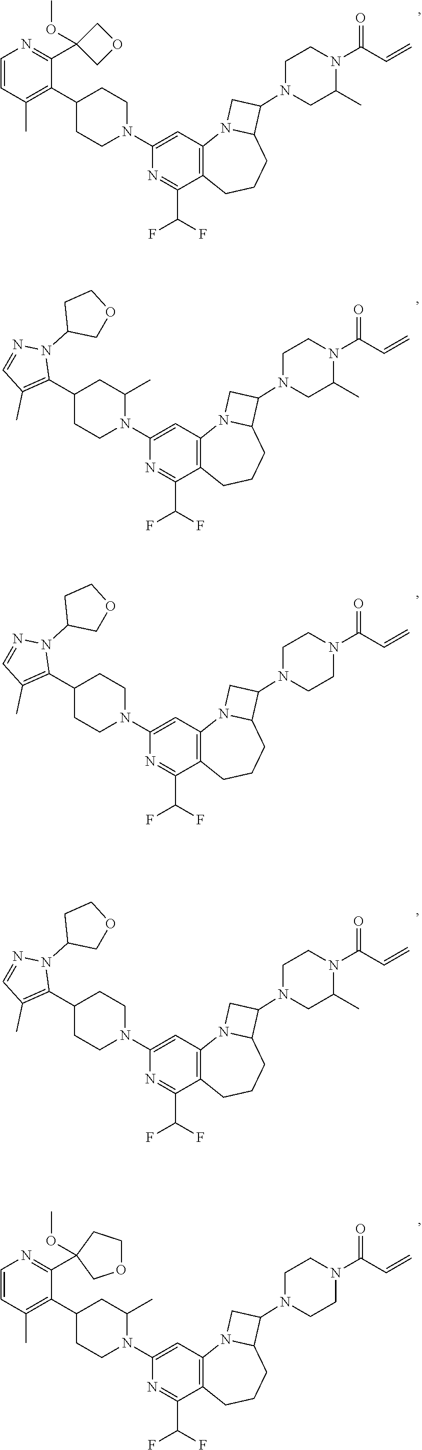 [SMILES: C=CC(=O)N1CCN(C2CN3c4cc(N5CCC(c6c(C)ccnc6C6(OC)COC6)CC5)nc(C(F)F)c4CCCC23)CC1C ; C=CC(=O)N1CCN(C2CN3c4cc(N5CCC(c6c(C)cnn6C6CCOC6)CC5C)nc(C(F)F)c4CCCC23)CC1C ; C=CC(=O)N1CCN(C2CN3c4cc(N5CCC(c6c(C)cnn6C6CCOC6)CC5)nc(C(F)F)c4CCCC23)CC1 ; C=CC(=O)N1CCN(C2CN3c4cc(N5CCC(c6c(C)cnn6C6CCOC6)CC5)nc(C(F)F)c4CCCC23)CC1C ; C=CC(=O)N1CCN(C2CN3c4cc(N5CCC(c6c(C)ccnc6C6(OC)CCOC6)CC5C)nc(C(F)F)c4CCCC23)CC1]

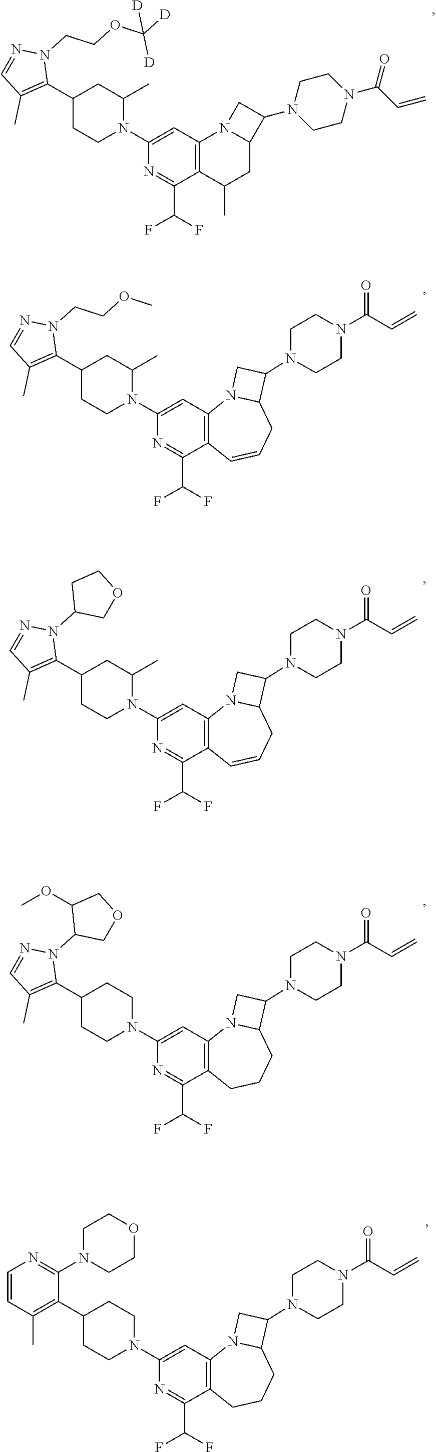 [SMILES: [2H]C([2H])([2H])OCCn1ncc(C)c1C1CCN(c2cc3c(c(C(F)F)n2)C(C)CC2C(N4CCN(C(=O)C=C)CC4)CN32)C(C)C1 ; C=CC(=O)N1CCN(C2CN3c4cc(N5CCC(c6c(C)cnn6CCOC)CC5C)nc(C(F)F)c4C=CCC23)CC1 ; C=CC(=O)N1CCN(C2CN3c4cc(N5CCC(c6c(C)cnn6C6CCOC6)CC5C)nc(C(F)F)c4C=CCC23)CC1 ; C=CC(=O)N1CCN(C2CN3c4cc(N5CCC(c6c(C)cnn6C6COCC6OC)CC5)nc(C(F)F)c4CCCC23)CC1 ; C=CC(=O)N1CCN(C2CN3c4cc(N5CCC(c6c(C)ccnc6N6CCOCC6)CC5)nc(C(F)F)c4CCCC23)CC1]

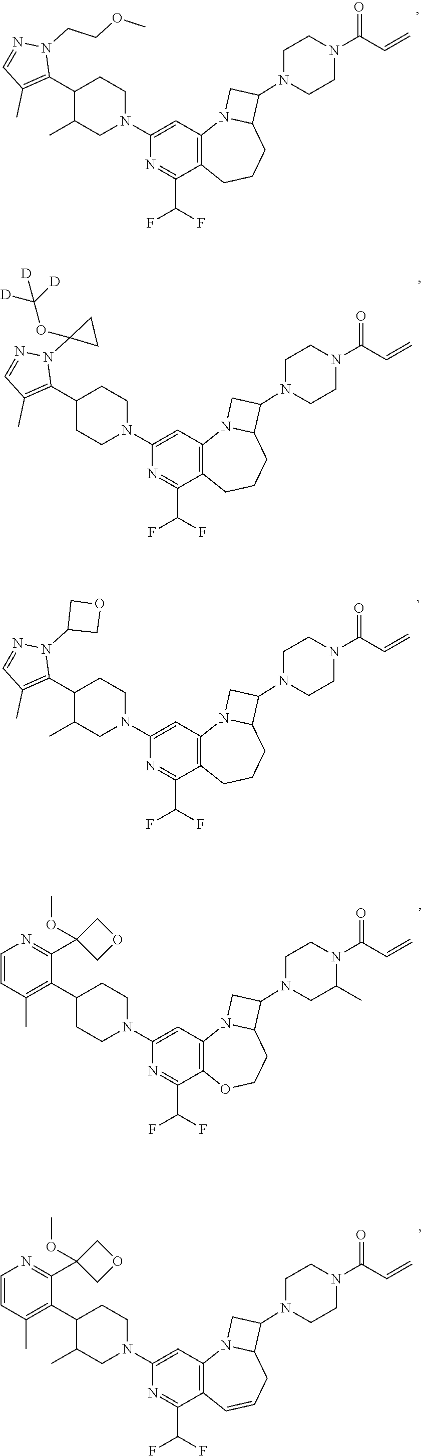 [SMILES: C=CC(=O)N1CCN(C2CN3c4cc(N5CCC(c6c(C)cnn6CCOC)C(C)C5)nc(C(F)F)c4CCCC23)CC1 ; [2H]C([2H])([2H])OC1(n2ncc(C)c2C2CCN(c3cc4c(c(C(F)F)n3)CCCC3C(N5CCN(C(=O)C=C)CC5)CN43)CC2)CC1 ; C=CC(=O)N1CCN(C2CN3c4cc(N5CCC(c6c(C)cnn6C6COC6)C(C)C5)nc(C(F)F)c4CCCC23)CC1 ; C=CC(=O)N1CCN(C2CN3c4cc(N5CCC(c6c(C)ccnc6C6(OC)COC6)CC5)nc(C(F)F)c4OCCC23)CC1C ; C=CC(=O)N1CCN(C2CN3c4cc(N5CCC(c6c(C)ccnc6C6(OC)COC6)C(C)C5)nc(C(F)F)c4C=CCC23)CC1]

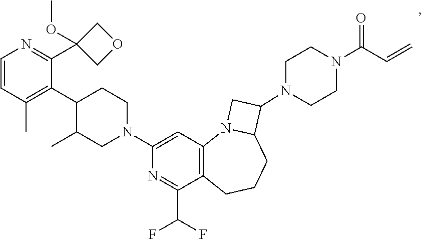 [SMILES: C=CC(=O)N1CCN(C2CN3c4cc(N5CCC(c6c(C)ccnc6C6(OC)COC6)C(C)C5)nc(C(F)F)c4CCCC23)CC1]

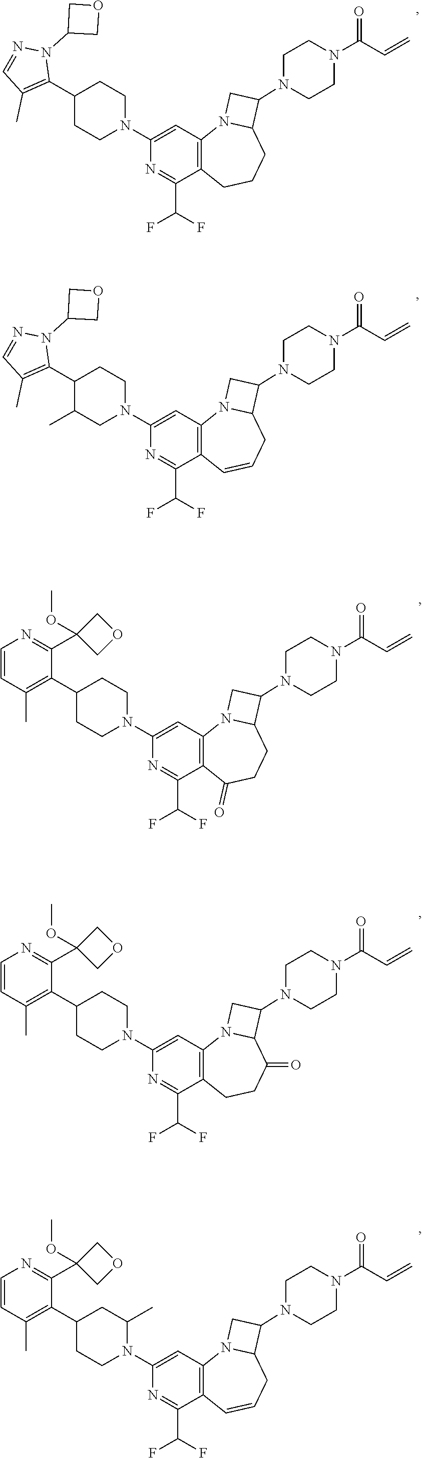 [SMILES: C=CC(=O)N1CCN(C2CN3c4cc(N5CCC(c6c(C)cnn6C6COC6)CC5)nc(C(F)F)c4CCCC23)CC1 ; C=CC(=O)N1CCN(C2CN3c4cc(N5CCC(c6c(C)cnn6C6COC6)C(C)C5)nc(C(F)F)c4C=CCC23)CC1 ; C=CC(=O)N1CCN(C2CN3c4cc(N5CCC(c6c(C)ccnc6C6(OC)COC6)CC5)nc(C(F)F)c4C(=O)CCC23)CC1 ; C=CC(=O)N1CCN(C2CN3c4cc(N5CCC(c6c(C)ccnc6C6(OC)COC6)CC5)nc(C(F)F)c4CCC(=O)C23)CC1 ; C=CC(=O)N1CCN(C2CN3c4cc(N5CCC(c6c(C)ccnc6C6(OC)COC6)CC5C)nc(C(F)F)c4C=CCC23)CC1]

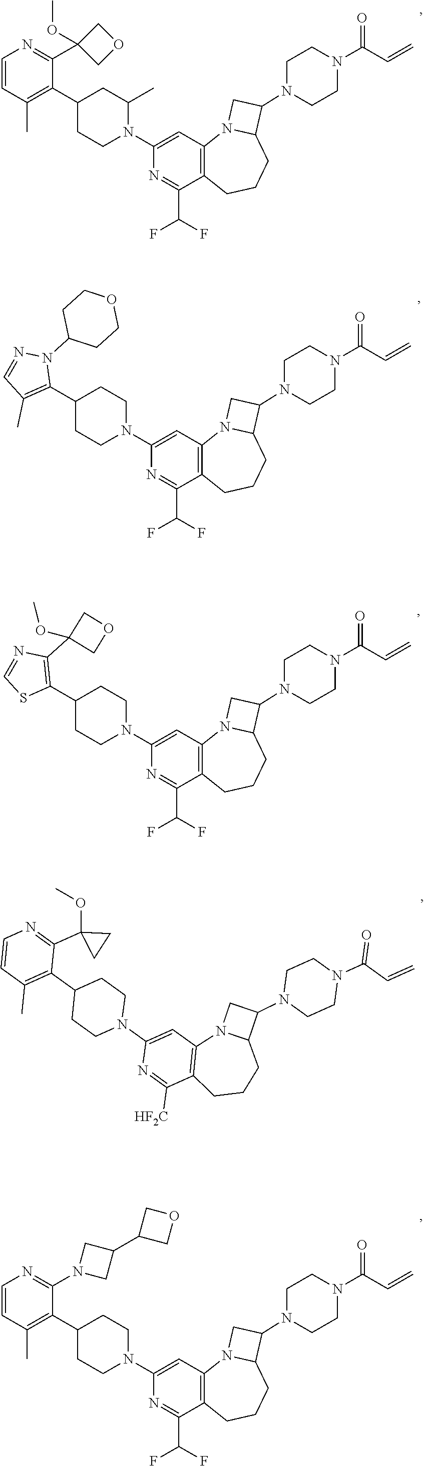

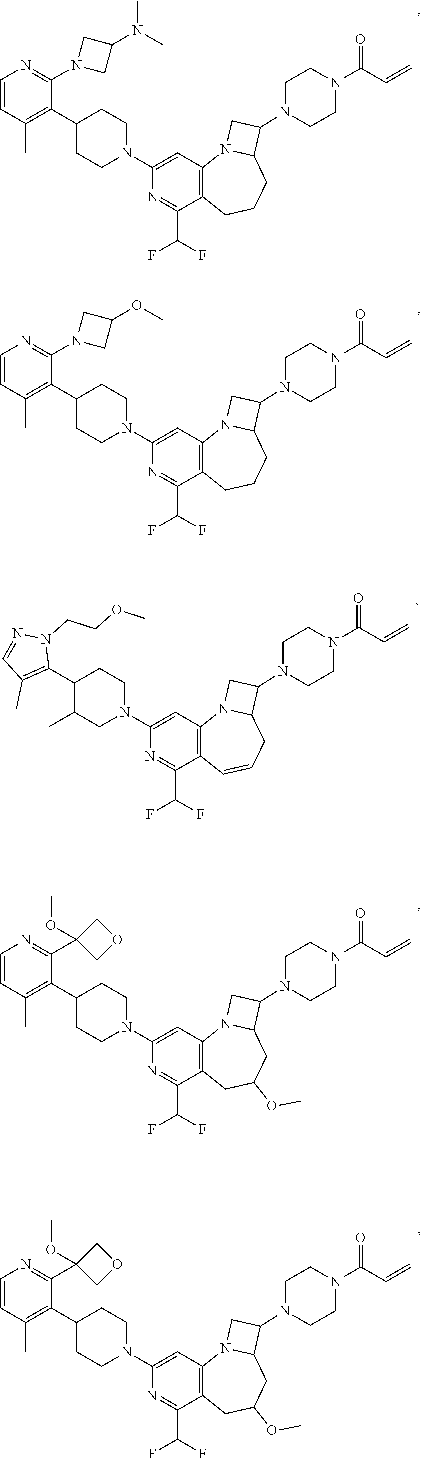 [SMILES: C=CC(=O)N1CCN(C2CN3c4cc(N5CCC(c6c(C)ccnc6N6CC(N(C)C)C6)CC5)nc(C(F)F)c4CCCC23)CC1 ; C=CC(=O)N1CCN(C2CN3c4cc(N5CCC(c6c(C)ccnc6N6CC(OC)C6)CC5)nc(C(F)F)c4CCCC23)CC1 ; C=CC(=O)N1CCN(C2CN3c4cc(N5CCC(c6c(C)cnn6CCOC)C(C)C5)nc(C(F)F)c4C=CCC23)CC1 ; C=CC(=O)N1CCN(C2CN3c4cc(N5CCC(c6c(C)ccnc6C6(OC)COC6)CC5)nc(C(F)F)c4CC(OC)CC23)CC1 ; C=CC(=O)N1CCN(C2CN3c4cc(N5CCC(c6c(C)ccnc6C6(OC)COC6)CC5)nc(C(F)F)c4CC(OC)CC23)CC1]

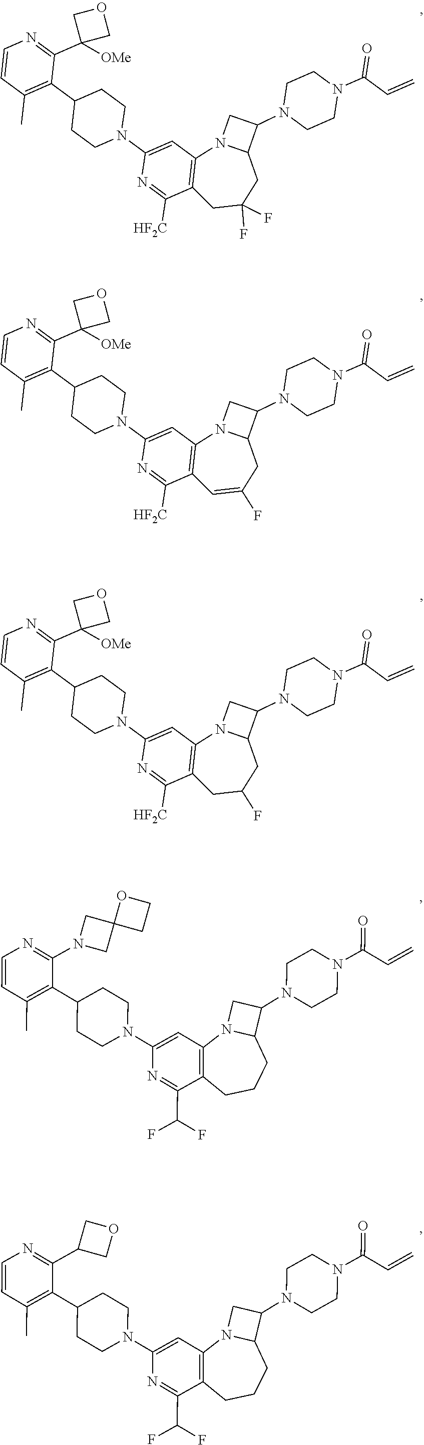

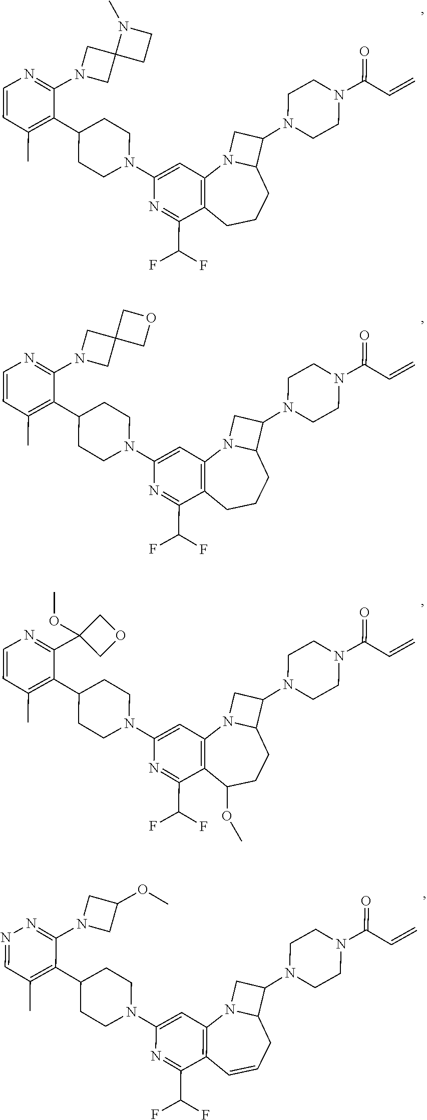 [SMILES: C=CC(=O)N1CCN(C2CN3c4cc(N5CCC(c6c(C)ccnc6N6CC7(CCN7C)C6)CC5)nc(C(F)F)c4CCCC23)CC1 ; C=CC(=O)N1CCN(C2CN3c4cc(N5CCC(c6c(C)ccnc6N6CC7(COC7)C6)CC5)nc(C(F)F)c4CCCC23)CC1 ; C=CC(=O)N1CCN(C2CN3c4cc(N5CCC(c6c(C)ccnc6C6(OC)COC6)CC5)nc(C(F)F)c4C(OC)CCC23)CC1 ; C=CC(=O)N1CCN(C2CN3c4cc(N5CCC(c6c(C)cnnc6N6CC(OC)C6)CC5)nc(C(F)F)c4C=CCC23)CC1]

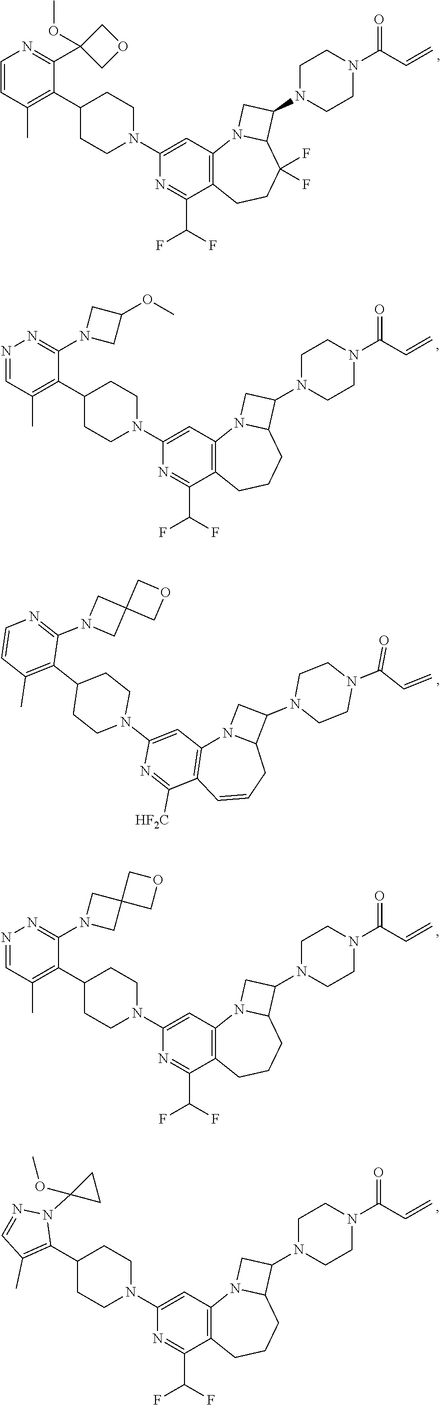

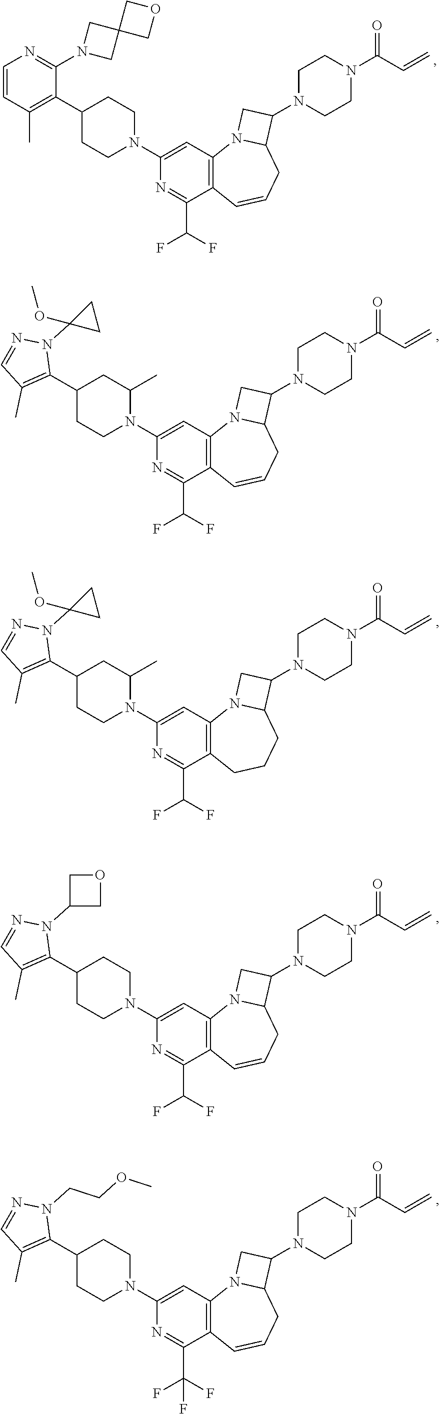 [SMILES: C=CC(=O)N1CCN(C2CN3c4cc(N5CCC(c6c(C)ccnc6N6CC7(COC7)C6)CC5)nc(C(F)F)c4C=CCC23)CC1 ; C=CC(=O)N1CCN(C2CN3c4cc(N5CCC(c6c(C)cnn6C6(OC)CC6)CC5C)nc(C(F)F)c4C=CCC23)CC1 ; C=CC(=O)N1CCN(C2CN3c4cc(N5CCC(c6c(C)cnn6C6(OC)CC6)CC5C)nc(C(F)F)c4CCCC23)CC1 ; C=CC(=O)N1CCN(C2CN3c4cc(N5CCC(c6c(C)cnn6C6COC6)CC5)nc(C(F)F)c4C=CCC23)CC1 ; C=CC(=O)N1CCN(C2CN3c4cc(N5CCC(c6c(C)cnn6CCOC)CC5)nc(C(F)(F)F)c4C=CCC23)CC1]

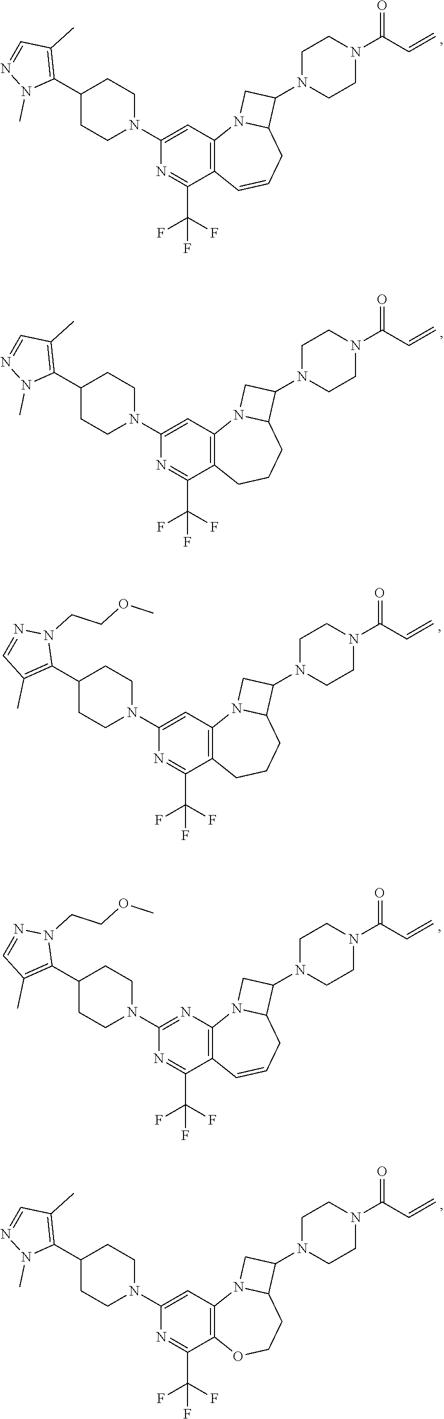 [SMILES: C=CC(=O)N1CCN(C2CN3c4cc(N5CCC(c6c(C)cnn6C)CC5)nc(C(F)(F)F)c4C=CCC23)CC1 ; C=CC(=O)N1CCN(C2CN3c4cc(N5CCC(c6c(C)cnn6C)CC5)nc(C(F)(F)F)c4CCCC23)CC1 ; C=CC(=O)N1CCN(C2CN3c4cc(N5CCC(c6c(C)cnn6CCOC)CC5)nc(C(F)(F)F)c4CCCC23)CC1 ; C=CC(=O)N1CCN(C2CN3c4nc(N5CCC(c6c(C)cnn6CCOC)CC5)nc(C(F)(F)F)c4C=CCC23)CC1 ; C=CC(=O)N1CCN(C2CN3c4cc(N5CCC(c6c(C)cnn6C)CC5)nc(C(F)(F)F)c4OCCC23)CC1]

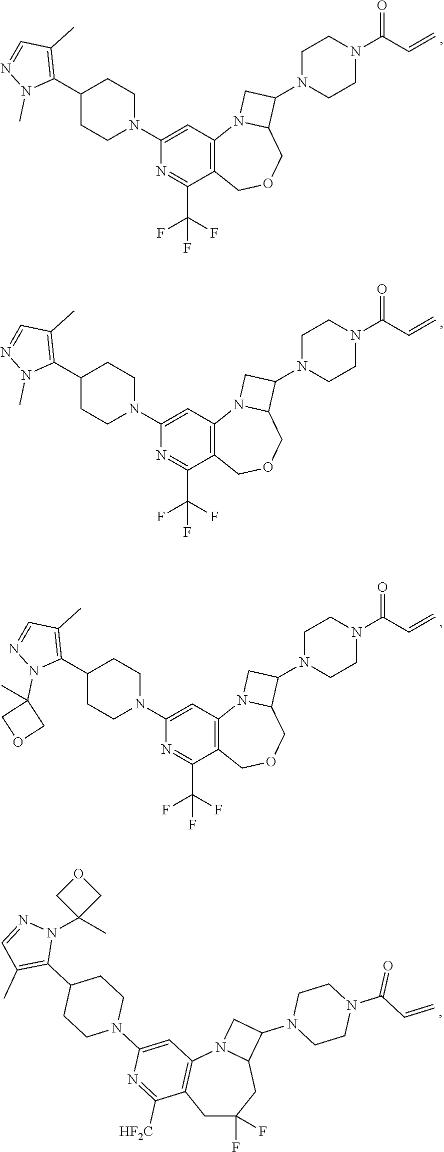

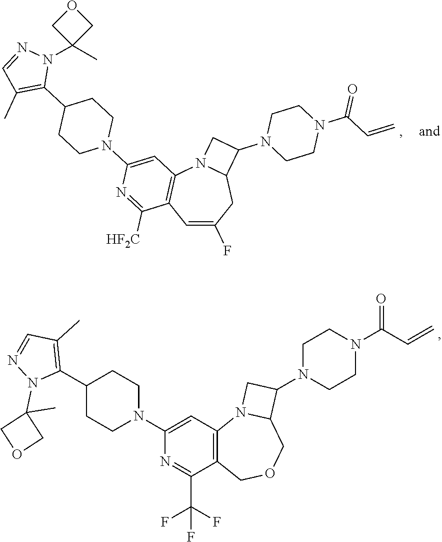

and pharmaceutically acceptable salts thereof.

Contemplated compounds of Formula (ILD) wherein Y is CH and Z is substituted pyrazolyl include, for example,

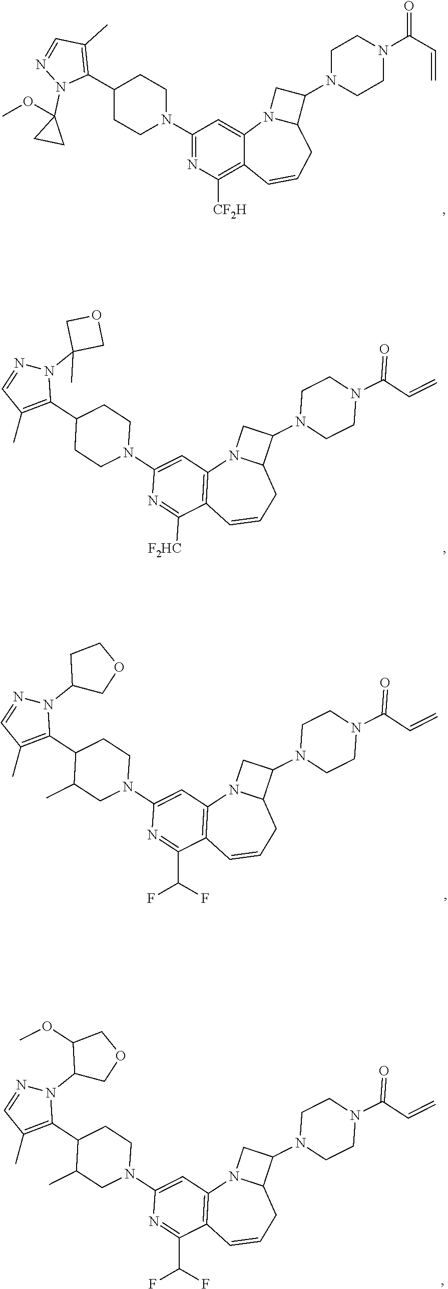 [SMILES: C=CC(=O)N1CCN(C2CN3c4cc(N5CCC(c6c(C)cnn6C6(OC)CC6)CC5)nc(C(F)F)c4C=CCC23)CC1 ; C=CC(=O)N1CCN(C2CN3c4cc(N5CCC(c6c(C)cnn6C6(C)COC6)CC5)nc(C(F)F)c4C=CCC23)CC1 ; C=CC(=O)N1CCN(C2CN3c4cc(N5CCC(c6c(C)cnn6C6CCOC6)C(C)C5)nc(C(F)F)c4C=CCC23)CC1 ; C=CC(=O)N1CCN(C2CN3c4cc(N5CCC(c6c(C)cnn6C6COCC6OC)C(C)C5)nc(C(F)F)c4C=CCC23)CC1]

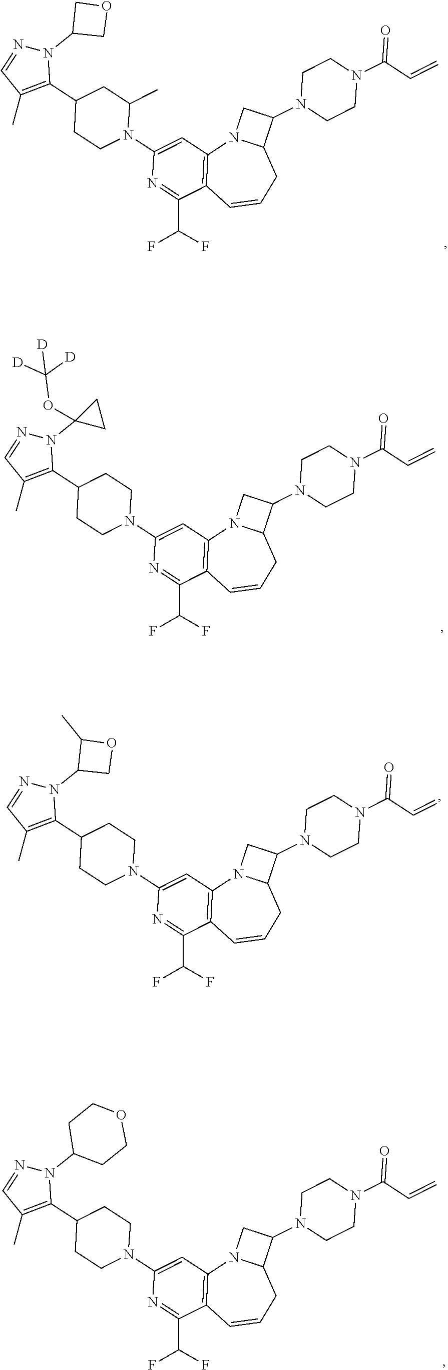

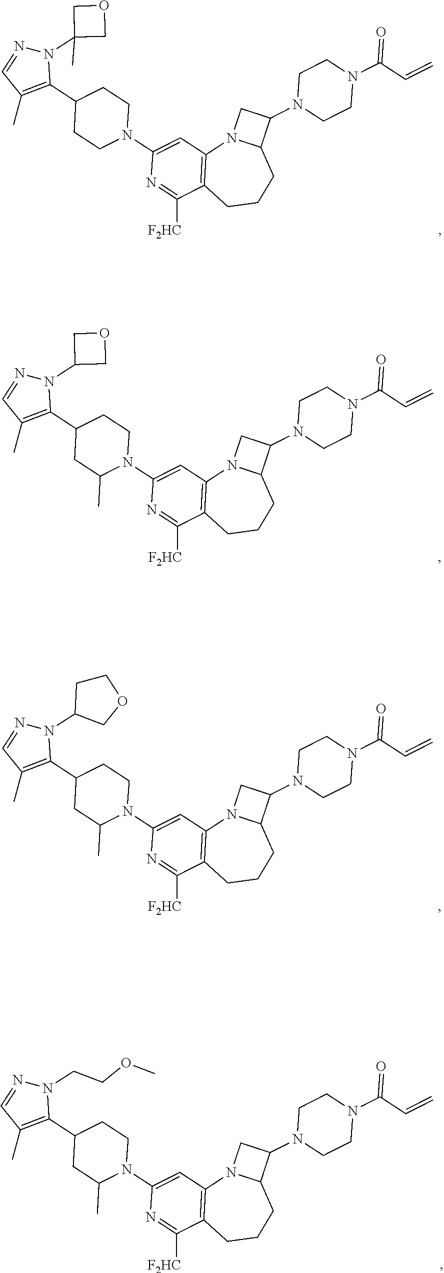 [SMILES: C=CC(=O)N1CCN(C2CN3c4cc(N5CCC(c6c(C)cnn6C6(C)COC6)CC5)nc(C(F)F)c4CCCC23)CC1 ; C=CC(=O)N1CCN(C2CN3c4cc(N5CCC(c6c(C)cnn6C6COC6)CC5C)nc(C(F)F)c4CCCC23)CC1 ; C=CC(=O)N1CCN(C2CN3c4cc(N5CCC(c6c(C)cnn6C6CCOC6)CC5C)nc(C(F)F)c4CCCC23)CC1 ; C=CC(=O)N1CCN(C2CN3c4cc(N5CCC(c6c(C)cnn6CCOC)CC5C)nc(C(F)F)c4CCCC23)CC1]

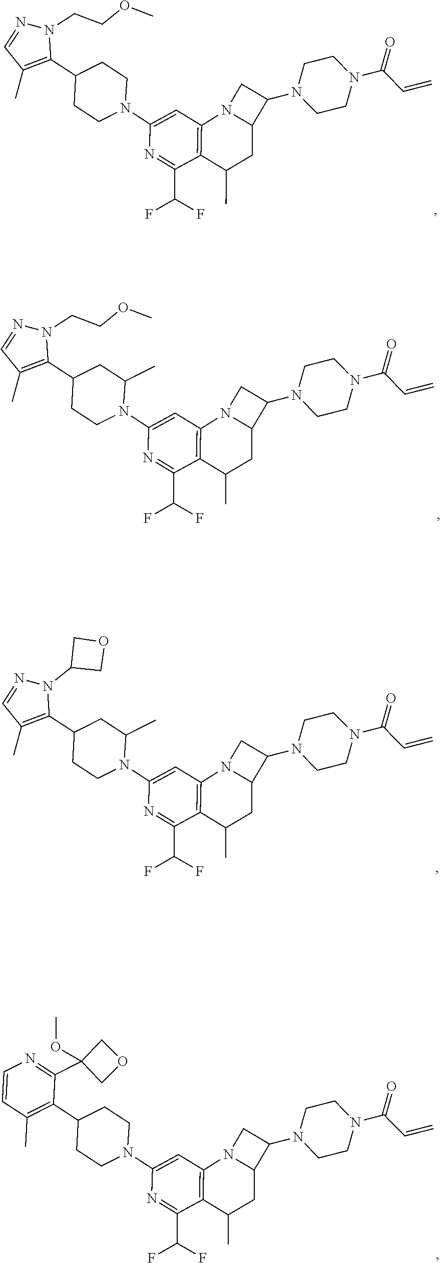 [SMILES: C=CC(=O)N1CCN(C2CN3c4cc(N5CCC(c6c(C)cnn6CCOC)CC5)nc(C(F)F)c4C(C)CC23)CC1 ; C=CC(=O)N1CCN(C2CN3c4cc(N5CCC(c6c(C)cnn6CCOC)CC5C)nc(C(F)F)c4C(C)CC23)CC1 ; C=CC(=O)N1CCN(C2CN3c4cc(N5CCC(c6c(C)cnn6C6COC6)CC5C)nc(C(F)F)c4C(C)CC23)CC1 ; C=CC(=O)N1CCN(C2CN3c4cc(N5CCC(c6c(C)ccnc6C6(OC)COC6)CC5)nc(C(F)F)c4C(C)CC23)CC1]

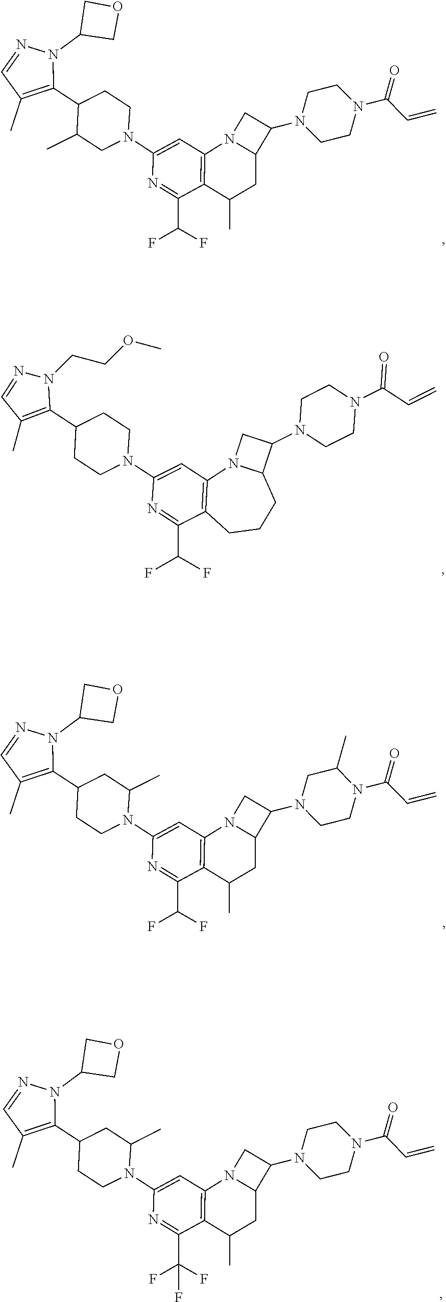 [SMILES: C=CC(=O)N1CCN(C2CN3c4cc(N5CCC(c6c(C)cnn6C6COC6)C(C)C5)nc(C(F)F)c4C(C)CC23)CC1 ; C=CC(=O)N1CCN(C2CN3c4cc(N5CCC(c6c(C)cnn6CCOC)CC5)nc(C(F)F)c4CCCC23)CC1 ; C=CC(=O)N1CCN(C2CN3c4cc(N5CCC(c6c(C)cnn6C6COC6)CC5C)nc(C(F)F)c4C(C)CC23)CC1C ; C=CC(=O)N1CCN(C2CN3c4cc(N5CCC(c6c(C)cnn6C6COC6)CC5C)nc(C(F)(F)F)c4C(C)CC23)CC1]

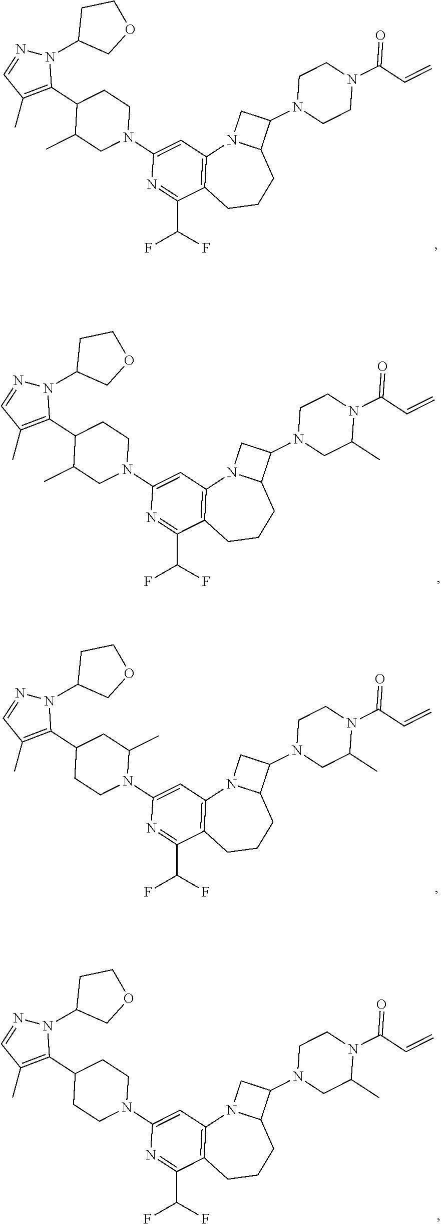 [SMILES: C=CC(=O)N1CCN(C2CN3c4cc(N5CCC(c6c(C)cnn6C6CCOC6)C(C)C5)nc(C(F)F)c4CCCC23)CC1 ; C=CC(=O)N1CCN(C2CN3c4cc(N5CCC(c6c(C)cnn6C6CCOC6)C(C)C5)nc(C(F)F)c4CCCC23)CC1C ; C=CC(=O)N1CCN(C2CN3c4cc(N5CCC(c6c(C)cnn6C6CCOC6)CC5C)nc(C(F)F)c4CCCC23)CC1C ; C=CC(=O)N1CCN(C2CN3c4cc(N5CCC(c6c(C)cnn6C6CCOC6)CC5)nc(C(F)F)c4CCCC23)CC1C]

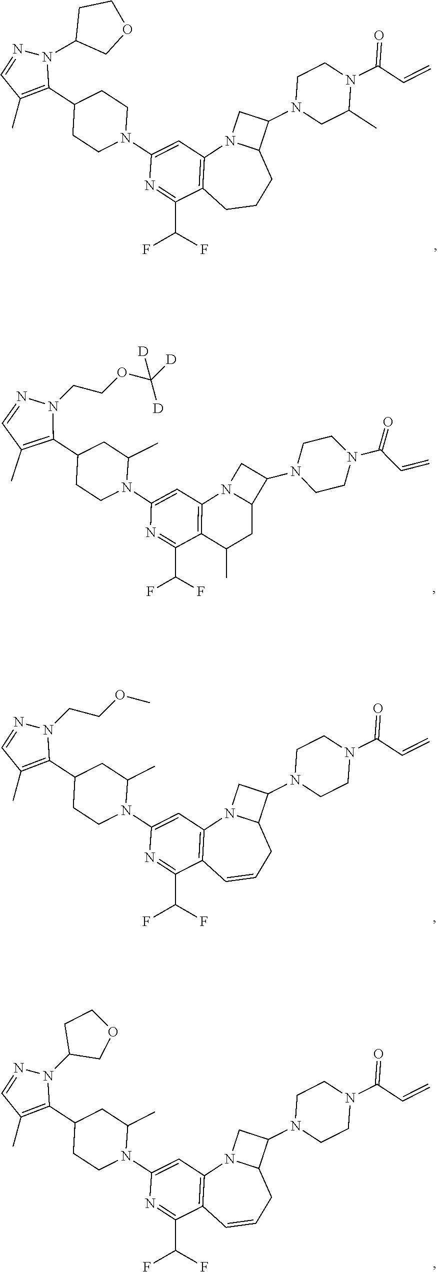 [SMILES: C=CC(=O)N1CCN(C2CN3c4cc(N5CCC(c6c(C)cnn6C6CCOC6)CC5)nc(C(F)F)c4CCCC23)CC1C ; [2H]C([2H])([2H])OCCn1ncc(C)c1C1CCN(c2cc3c(c(C(F)F)n2)C(C)CC2C(N4CCN(C(=O)C=C)CC4)CN32)C(C)C1 ; C=CC(=O)N1CCN(C2CN3c4cc(N5CCC(c6c(C)cnn6CCOC)CC5C)nc(C(F)F)c4C=CCC23)CC1 ; C=CC(=O)N1CCN(C2CN3c4cc(N5CCC(c6c(C)cnn6C6CCOC6)CC5C)nc(C(F)F)c4C=CCC23)CC1]

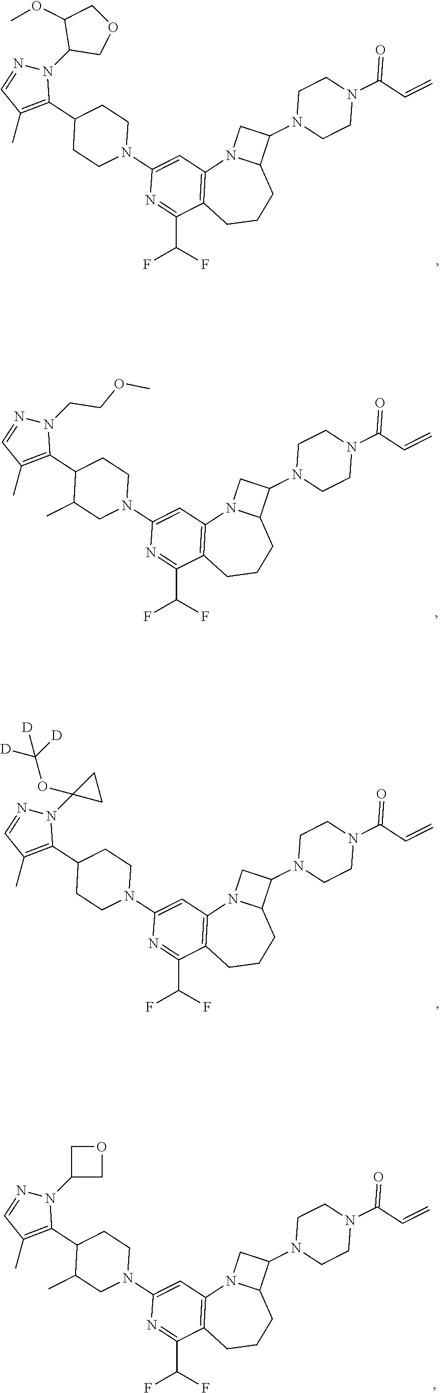 [SMILES: C=CC(=O)N1CCN(C2CN3c4cc(N5CCC(c6c(C)cnn6C6COCC6OC)CC5)nc(C(F)F)c4CCCC23)CC1 ; C=CC(=O)N1CCN(C2CN3c4cc(N5CCC(c6c(C)cnn6CCOC)C(C)C5)nc(C(F)F)c4CCCC23)CC1 ; [2H]C([2H])([2H])OC1(n2ncc(C)c2C2CCN(c3cc4c(c(C(F)F)n3)CCCC3C(N5CCN(C(=O)C=C)CC5)CN43)CC2)CC1 ; C=CC(=O)N1CCN(C2CN3c4cc(N5CCC(c6c(C)cnn6C6COC6)C(C)C5)nc(C(F)F)c4CCCC23)CC1]

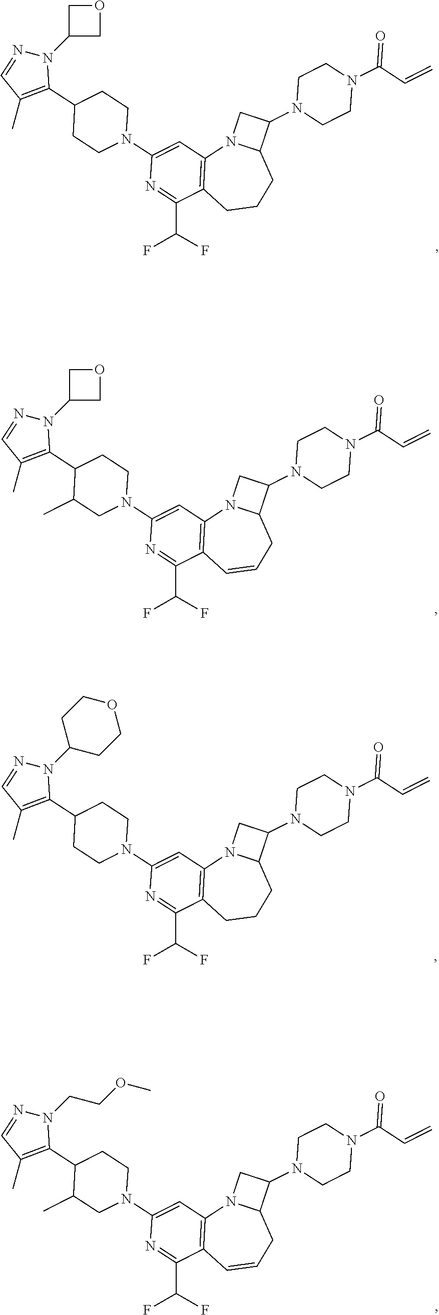 [SMILES: C=CC(=O)N1CCN(C2CN3c4cc(N5CCC(c6c(C)cnn6C6COC6)CC5)nc(C(F)F)c4CCCC23)CC1 ; C=CC(=O)N1CCN(C2CN3c4cc(N5CCC(c6c(C)cnn6C6COC6)C(C)C5)nc(C(F)F)c4C=CCC23)CC1 ; C=CC(=O)N1CCN(C2CN3c4cc(N5CCC(c6c(C)cnn6C6CCOCC6)CC5)nc(C(F)F)c4CCCC23)CC1 ; C=CC(=O)N1CCN(C2CN3c4cc(N5CCC(c6c(C)cnn6CCOC)C(C)C5)nc(C(F)F)c4C=CCC23)CC1]

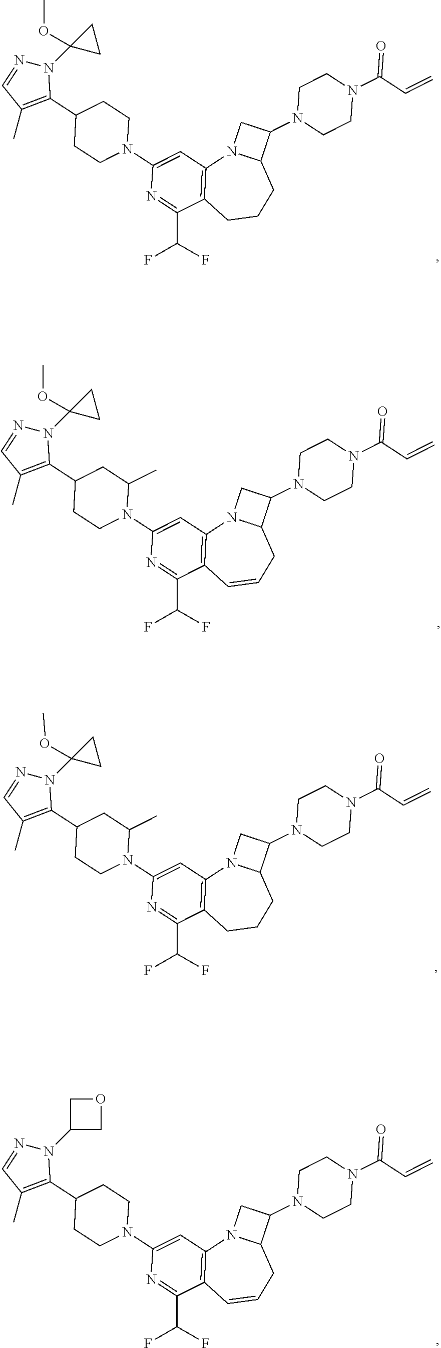 [SMILES: C=CC(=O)N1CCN(C2CN3c4cc(N5CCC(c6c(C)cnn6C6(OC)CC6)CC5)nc(C(F)F)c4CCCC23)CC1 ; C=CC(=O)N1CCN(C2CN3c4cc(N5CCC(c6c(C)cnn6C6(OC)CC6)CC5C)nc(C(F)F)c4C=CCC23)CC1 ; C=CC(=O)N1CCN(C2CN3c4cc(N5CCC(c6c(C)cnn6C6(OC)CC6)CC5C)nc(C(F)F)c4CCCC23)CC1 ; C=CC(=O)N1CCN(C2CN3c4cc(N5CCC(c6c(C)cnn6C6COC6)CC5)nc(C(F)F)c4C=CCC23)CC1]

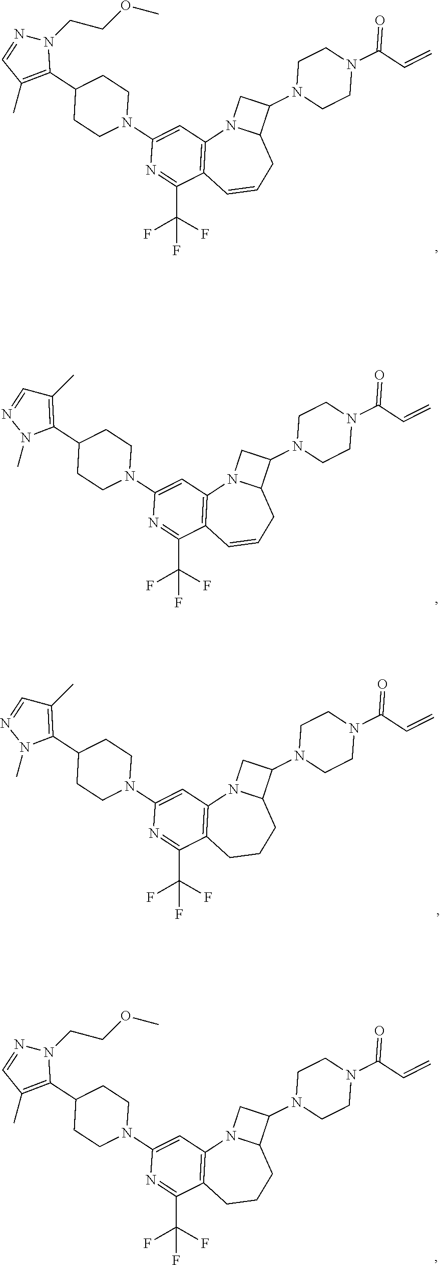 [SMILES: C=CC(=O)N1CCN(C2CN3c4cc(N5CCC(c6c(C)cnn6CCOC)CC5)nc(C(F)(F)F)c4C=CCC23)CC1 ; C=CC(=O)N1CCN(C2CN3c4cc(N5CCC(c6c(C)cnn6C)CC5)nc(C(F)(F)F)c4C=CCC23)CC1 ; C=CC(=O)N1CCN(C2CN3c4cc(N5CCC(c6c(C)cnn6C)CC5)nc(C(F)(F)F)c4CCCC23)CC1 ; C=CC(=O)N1CCN(C2CN3c4cc(N5CCC(c6c(C)cnn6CCOC)CC5)nc(C(F)(F)F)c4CCCC23)CC1]

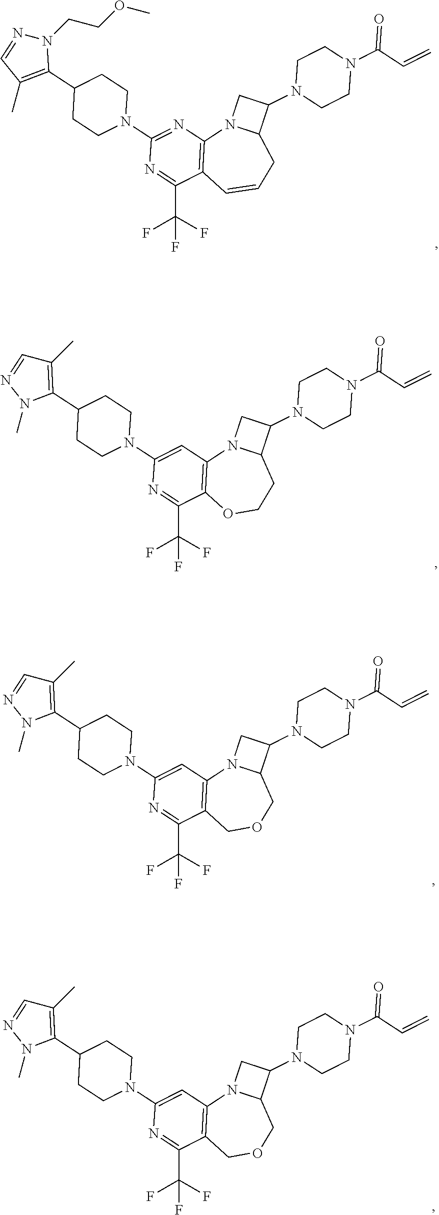 [SMILES: C=CC(=O)N1CCN(C2CN3c4nc(N5CCC(c6c(C)cnn6CCOC)CC5)nc(C(F)(F)F)c4C=CCC23)CC1 ; C=CC(=O)N1CCN(C2CN3c4cc(N5CCC(c6c(C)cnn6C)CC5)nc(C(F)(F)F)c4OCCC23)CC1 ; C=CC(=O)N1CCN(C2CN3c4cc(N5CCC(c6c(C)cnn6C)CC5)nc(C(F)(F)F)c4COCC23)CC1 ; C=CC(=O)N1CCN(C2CN3c4cc(N5CCC(c6c(C)cnn6C)CC5)nc(C(F)(F)F)c4COCC23)CC1]

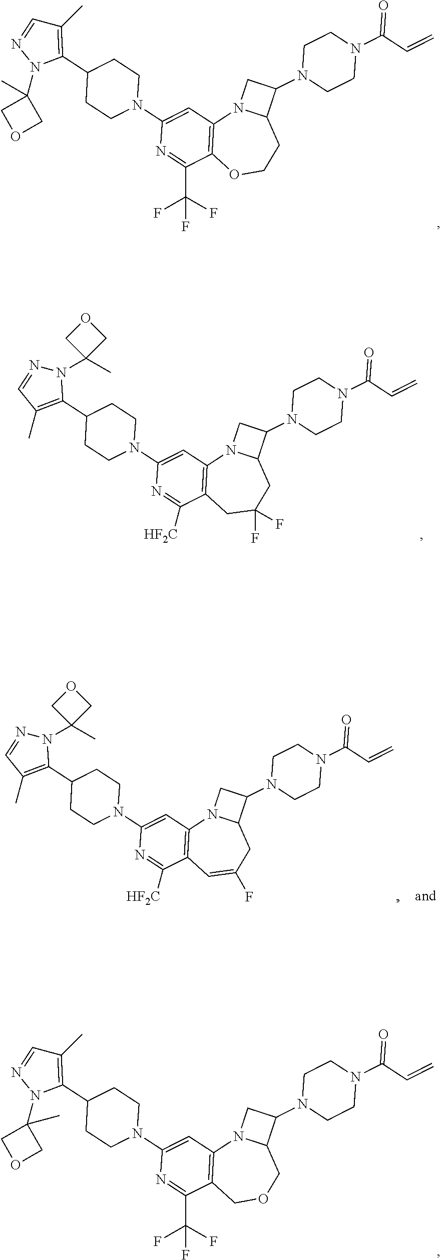

and pharmaceutically acceptable salts thereof. Contemplated compounds of Formula (IID) wherein Y is CH and Z is substituted thiazolyl include, for example,

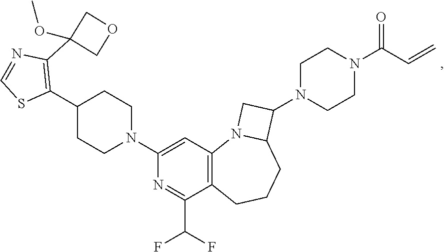 [SMILES: C=CC(=O)N1CCN(C2CN3c4cc(N5CCC(c6scnc6C6(OC)COC6)CC5)nc(C(F)F)c4CCCC23)CC1]

and pharmaceutically acceptable salts thereof. Contemplated compounds of Formula (IID) wherein Y is CH and Z is substituted pyridyl include, for example:

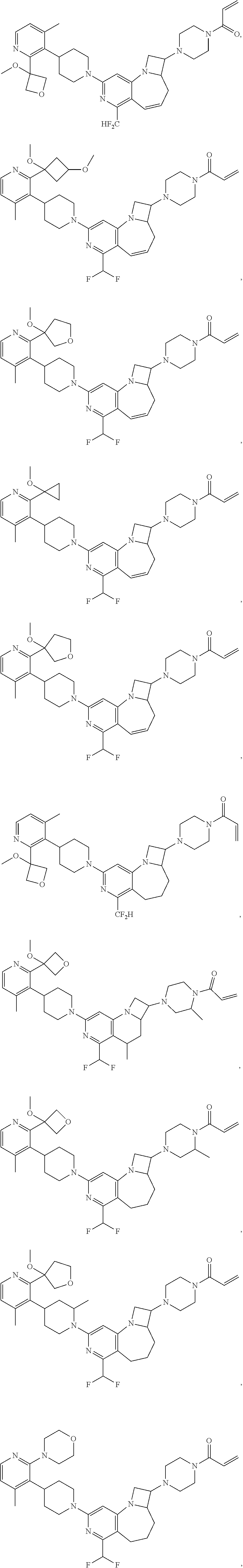

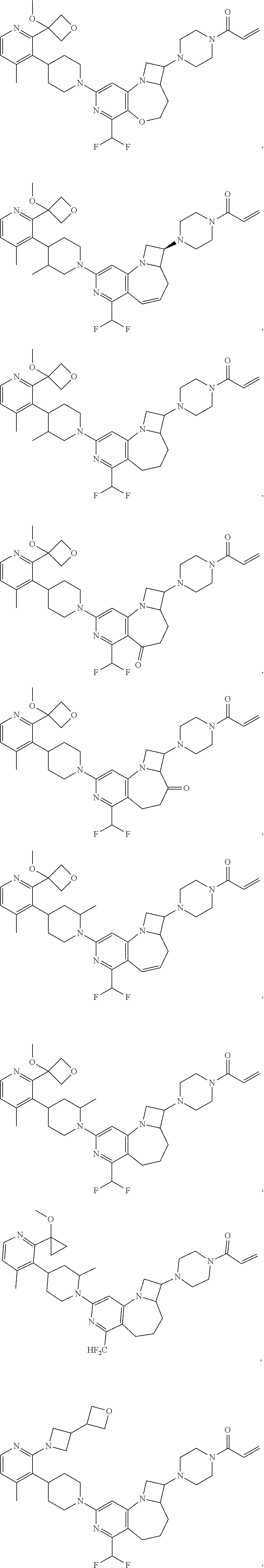

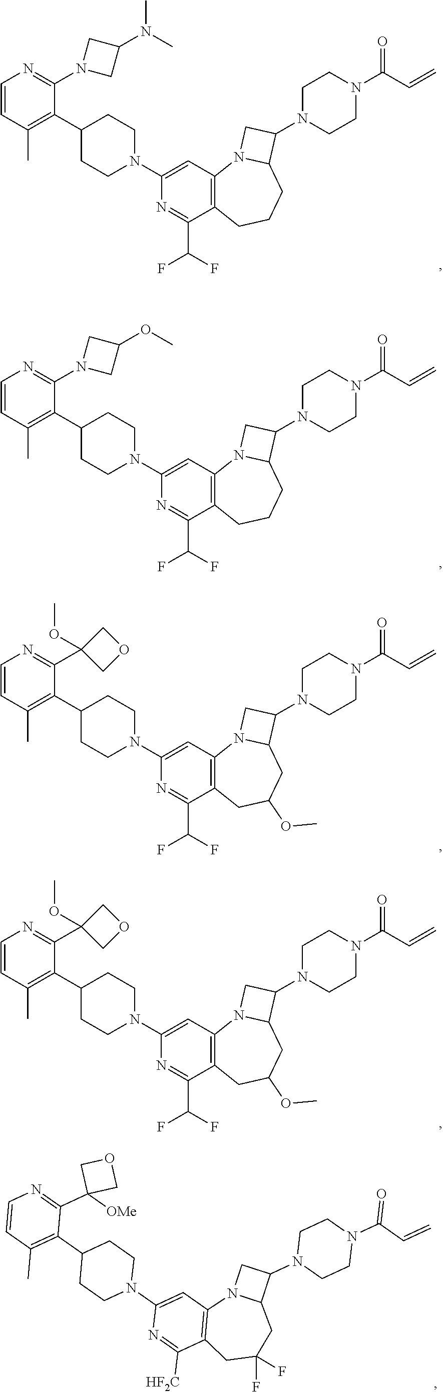

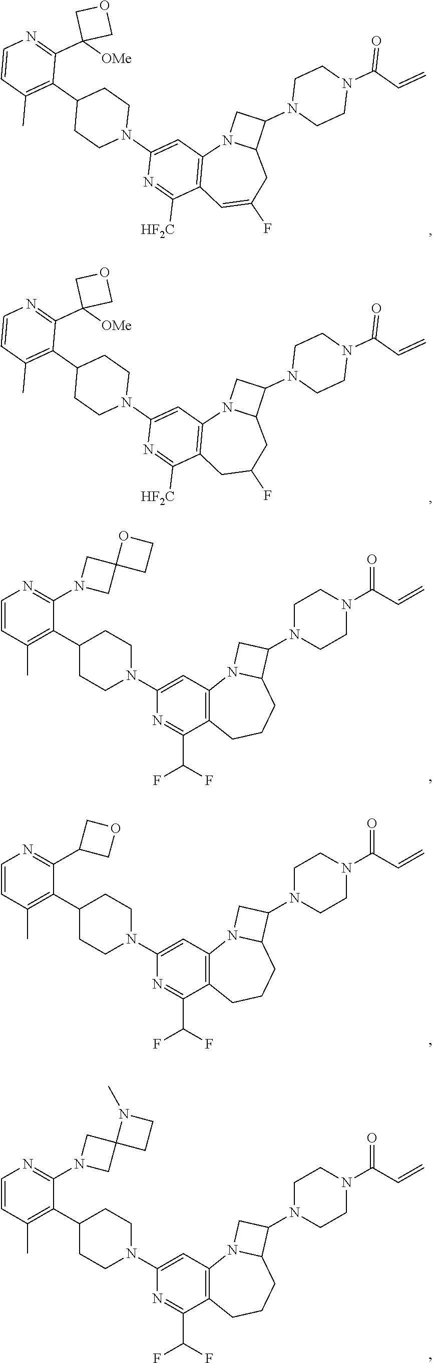

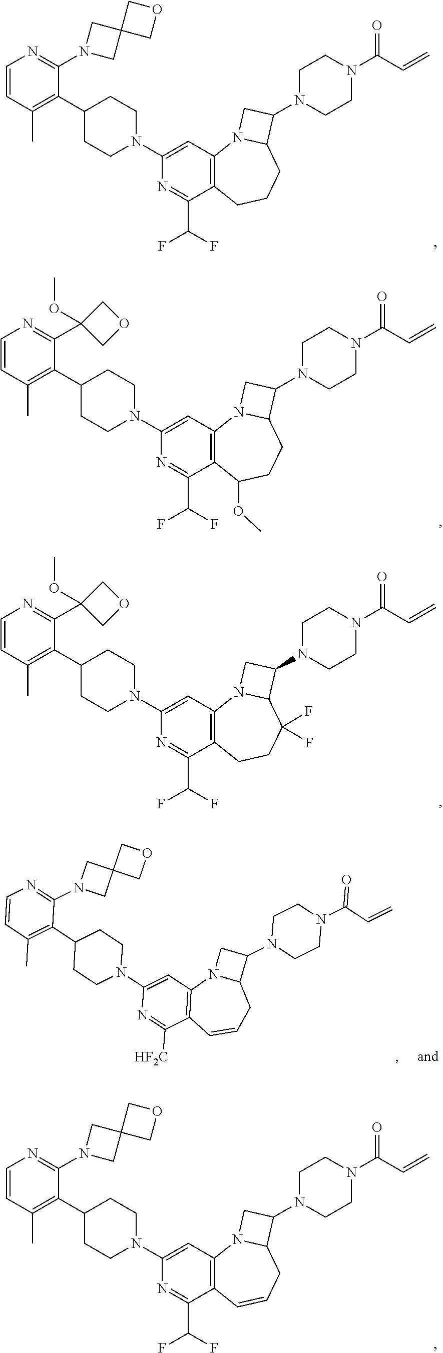

and pharmaceutically acceptable salts thereof. Contemplated compounds of Formula (IID) wherein Y is CH and Z is substituted pyridazinyl include, for example:

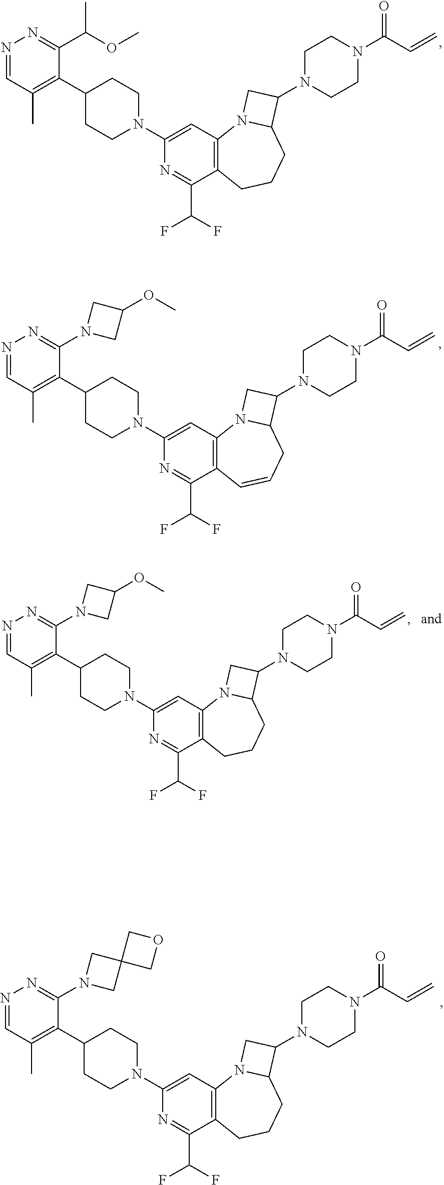 [SMILES: C=CC(=O)N1CCN(C2CN3c4cc(N5CCC(c6c(C)cnnc6C(C)OC)CC5)nc(C(F)F)c4CCCC23)CC1 ; C=CC(=O)N1CCN(C2CN3c4cc(N5CCC(c6c(C)cnnc6N6CC(OC)C6)CC5)nc(C(F)F)c4C=CCC23)CC1 ; C=CC(=O)N1CCN(C2CN3c4cc(N5CCC(c6c(C)cnnc6N6CC(OC)C6)CC5)nc(C(F)F)c4CCCC23)CC1 ; C=CC(=O)N1CCN(C2CN3c4cc(N5CCC(c6c(C)cnnc6N6CC7(COC7)C6)CC5)nc(C(F)F)c4CCCC23)CC1]

and pharmaceutically acceptable salts thereof. Contemplated compounds of Formula (IID) wherein Y is N

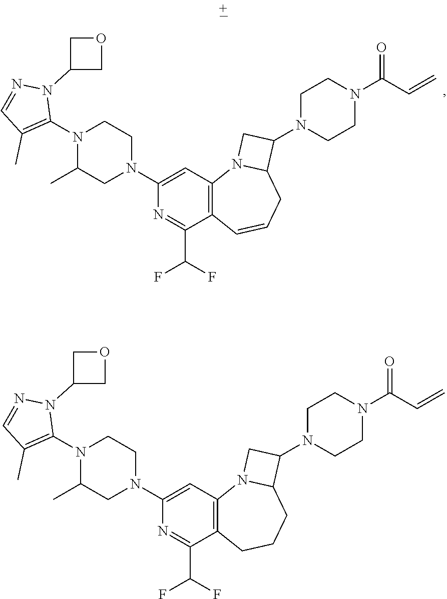 [SMILES: C=CC(=O)N1CCN(C2CN3c4cc(N5CCN(c6c(C)cnn6C6COC6)C(C)C5)nc(C(F)F)c4C=CCC23)CC1 ; C=CC(=O)N1CCN(C2CN3c4cc(N5CCN(c6c(C)cnn6C6COC6)C(C)C5)nc(C(F)F)c4CCCC23)CC1]

include, for example, and pharmaceutically acceptable salts thereof.

In some cases, Formula (II) has a structure of Formula (HIE). Contemplated compounds of Formula (IIE) include, for example,

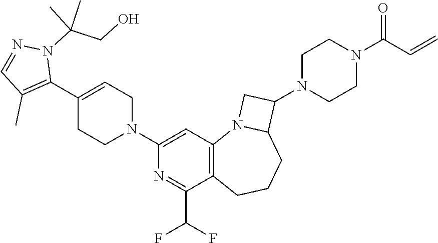 [SMILES: C=CC(=O)N1CCN(C2CN3c4cc(N5CC=C(c6c(C)cnn6C(C)(C)CO)CC5)nc(C(F)F)c4CCCC23)CC1]

and pharmaceutically acceptable salts thereof.

In some cases, the compound of Formula (II) is a compound listed in Table B, or a pharmaceutically acceptable salt thereof.

TABLE B

| Chemical Structure | Name |
|---|---|
| 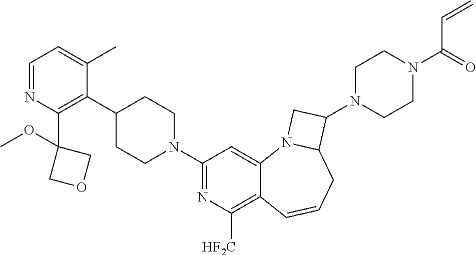 | 1-(4-(-4-(difluoromethyl)-2-(4-(2-(3-methoxy- 3-oxetanyl)-4-methyl-3-pyridinyl)-1- piperidinyl)-7,7a,8,9-tetrahydroazeto[1,2- a]pyrido[3,4-f]azepin-8-yl)-1-piperazinyl)-2- propen-1-one |
| 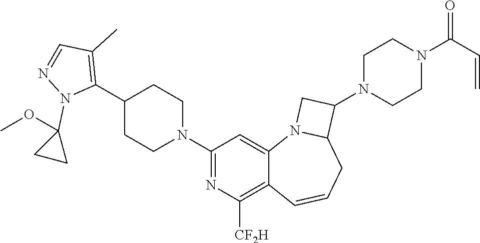 [SMILES: C=CC(=O)N1CCN(C2CN3c4cc(N5CCC(c6c(C)cnn6C6(OC)CC6)CC5)nc(C(F)F)c4C=CCC23)CC1] | 1-(4-(-4-(difluoromethyl)-2-(4-(1-(1- methoxycyclopropyl)-4-methyl-1H-pyrazol-5- yl)-1-piperidinyl)-7,7a,8,9-tetrahydroazeto[1,2- a]pyrido[3,4-f]azepin-8-yl)-1-piperazinyl)-2- propen-1-one |
| 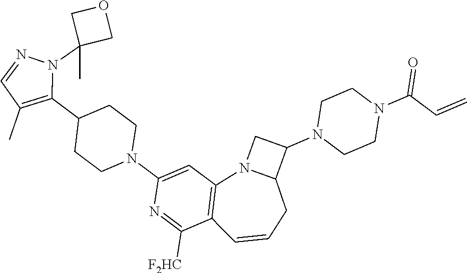 [SMILES: C=CC(=O)N1CCN(C2CN3c4cc(N5CCC(c6c(C)cnn6C6(C)COC6)CC5)nc(C(F)F)c4C=CCC23)CC1] | 1-(4-(-4-(difluoromethyl)-2-(4-(4-methyl-1-(3- methyl-3-oxetanyl)-1H-pyrazol-5-yl)-1- piperidinyl)-7,7a,8,9-tetrahydroazeto[1,2- a]pyrido[3,4-f]azepin-8-yl)-1-piperazinyl)-2- propen-1-one |
| 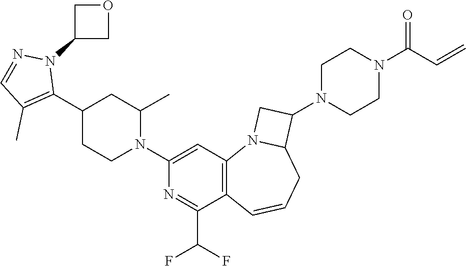 | 1-(4-(4-(difluoromethyl)-2-(2-methyl-4-(4- methyl-1-(3-oxetanyl)-1H-pyrazol-5-yl)-1- piperidinyl)-7,7a,8,9-tetrahydroazeto[1,2- a]pyrido[3,4-f]azepin-8-yl)-1-piperazinyl)-2- propen-1-one |
| 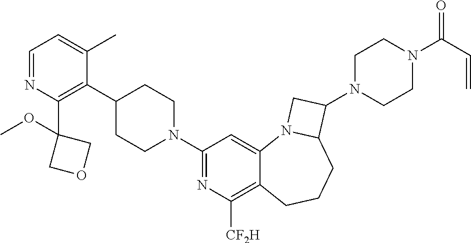 [SMILES: C=CC(=O)N1CCN(C2CN3c4cc(N5CCC(c6c(C)ccnc6C6(OC)COC6)CC5)nc(C(F)F)c4CCCC23)CC1] | 1-(4-(4-(difluoromethyl)-2-(4-(2-(3-methoxy-3- oxetanyl)-4-methyl-3-pyridinyl)-1-piperidinyl)- 5,6,7,7a,8,9-hexahydroazeto[1,2-a]pyrido[3,4- f]azepin-8-yl)-1-piperazinyl)-2-propen-1-one |
| 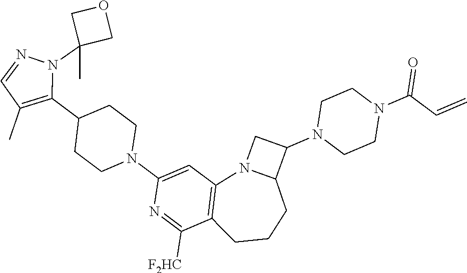 [SMILES: C=CC(=O)N1CCN(C2CN3c4cc(N5CCC(c6c(C)cnn6C6(C)COC6)CC5)nc(C(F)F)c4CCCC23)CC1] | 1-(4-(4-(difluoromethyl)-2-(4-(4-methyl-1-(3- methyloxetan-3-yl)-1H-pyrazol-5-yl)piperidin- 1-yl)-5,6,7,7a,8,9-hexahydroazeto[1,2- a]pyrido[3,4-f]azepin-8-yl)piperazin-1-yl)prop- 2-en-1-one |
| 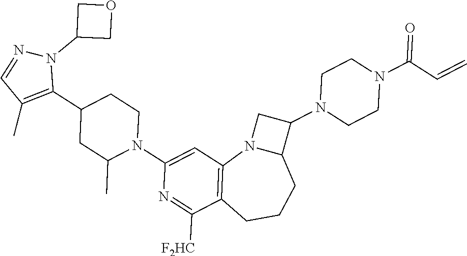 [SMILES: C=CC(=O)N1CCN(C2CN3c4cc(N5CCC(c6c(C)cnn6C6COC6)CC5C)nc(C(F)F)c4CCCC23)CC1] | 1-(4-(4-(difluoromethyl)-2-(2-methyl-4-(4- methyl-1-(3-oxetanyl)-1H-pyrazol-5-yl)-1- piperidinyl)-5,6,7,7a,8,9-hexahydroazeto[1,2- a]pyrido[3,4-f]azepin-8-yl)-1-piperazinyl)-2- propen-1-one |
| 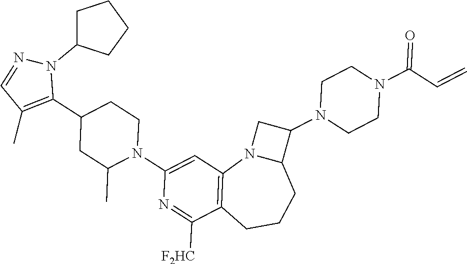 [SMILES: C=CC(=O)N1CCN(C2CN3c4cc(N5CCC(c6c(C)cnn6C6CCCC6)CC5C)nc(C(F)F)c4CCCC23)CC1] | 1-(4-(4-(difluoromethyl)-2-(2-methyl-4-(4- methyl-1-(tetrahydro-3-furanyl)-1H-pyrazol-5- yl)-1-piperidinyl)-5,6,7,7a,8,9- hexahydroazeto[1,2-a]pyrido[3,4-f]azepin-8-yl)- 1-piperazinyl)-2-propen-1-one |
| 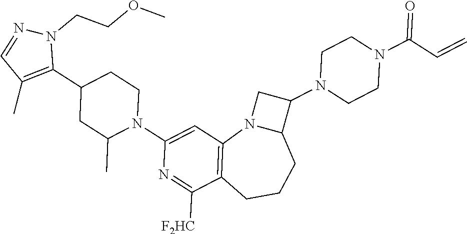 [SMILES: C=CC(=O)N1CCN(C2CN3c4cc(N5CCC(c6c(C)cnn6CCOC)CC5C)nc(C(F)F)c4CCCC23)CC1] | 1-(4-(4-(difluoromethyl)-2-(4-(1-(2- methoxyethyl)-4-methyl-1H-pyrazol-5-yl)-2- methyl-1-piperidinyl)-5,6,7,7a,8,9- hexahydroazeto[1,2-a]pyrido[3,4-f]azepin-8-yl)- 1-piperazinyl)-2-propen-1-one |
| 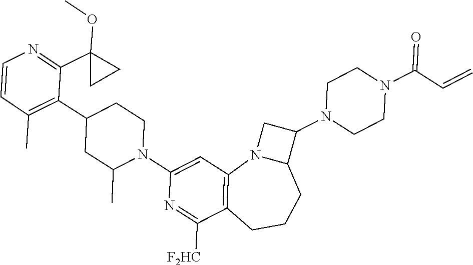 [SMILES: C=CC(=O)N1CCN(C2CN3c4cc(N5CCC(c6c(C)ccnc6C6(OC)CC6)CC5C)nc(C(F)F)c4CCCC23)CC1] | 1-(4-(4-(difluoromethyl)-2-(4-(2-(1- methoxycyclopropyl)-4-methyl-3-pyridinyl)-1- piperidinyl)-5,6,7,7a,8,9-hexahydroazeto[1,2- a]pyrido[3,4-f]azepin-8-yl)-1-piperazinyl)-2- propen-1-one |
| 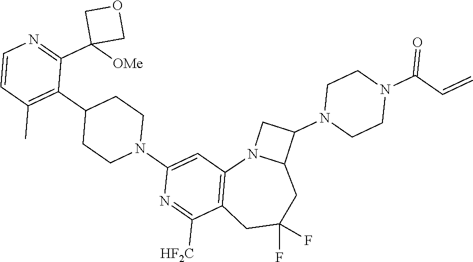 | 1-(4-(4-(difluoromethyl)-6,6-difluoro-2-(4-(2- (3-methoxy-3-oxetanyl)-4-methyl-3-pyridinyl)- 1-piperidinyl)-5,6,7,7a,8,9-hexahydroazeto[1,2- a]pyrido[3,4-f]azepin-8-yl)-1-piperazinyl)-2- propen-1-one |
| 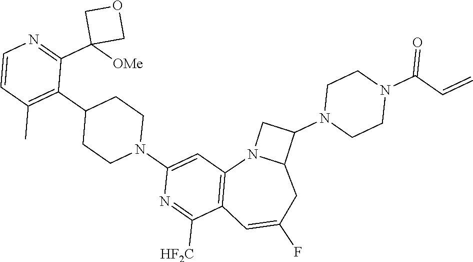 | 1-(4-(4-(difluoromethyl)-6-fluoro-2-(4-(2-(3- methoxy-3-oxetanyl)-4-methyl-3-pyridinyl)-1- piperidinyl)-7,7a,8,9-tetrahydroazeto[1,2- a]pyrido[3,4-f]azepin-8-yl)-1-piperazinyl)-2- propen-1-one |
| 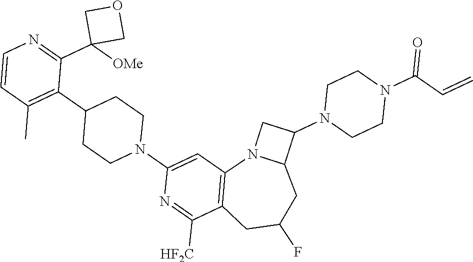 | 1-(4-(4-(difluoromethyl)-6-fluoro-2-(4-(2-(3- methoxy-3-oxetanyl)-4-methyl-3-pyridinyl)-1- piperidinyl)-5,6,7,7a,8,9-hexahydroazeto[1,2- a]pyrido[3,4-f]azepin-8-yl)-1-piperazinyl)-2- propen-1-one |
| 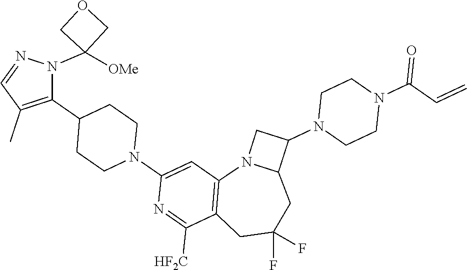 | 1-(4-(4-(difluoromethyl)-6,6-difluoro-2-(4-(4- methyl-1-(3-methyloxetan-3-yl)-1H-pyrazol-5- yl)piperidin-1-yl)-5,6,7,7a,8,9- hexahydroazeto[1,2-a]pyrido[3,4-f]azepin-8- yl)piperazin-1-yl)prop-2-en-1-one |
| 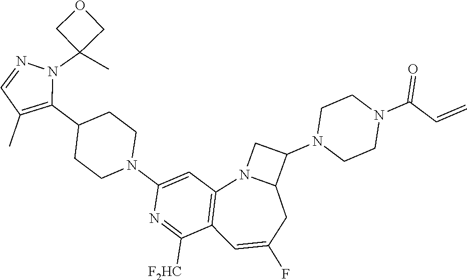 [SMILES: C=CC(=O)N1CCN(C2CN3c4cc(N5CCC(c6c(C)cnn6C6(C)COC6)CC5)nc(C(F)F)c4C=C(F)CC23)CC1] | 1-(4-(4-(difluoromethyl)-6-fluoro-2-(4-(4- methyl-1-(3-methyloxetan-3-yl)-1H-pyrazol-5- yl)piperidin-1-yl)-7,7a,8,9-tetrahydroazeto[1,2- a]pyrido[3,4-f]azepin-8-yl)piperazin-1-yl)prop- 2-en-1-one |

In some cases, the compound of Formula (II) is

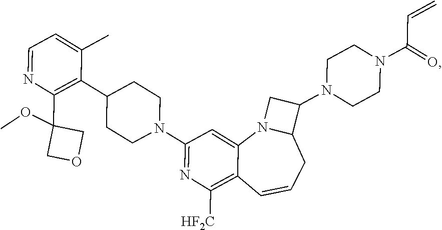

or a pharmaceutically acceptable salt thereof. In some cases, the compound of Formula (II) is

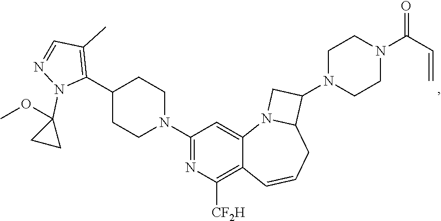 [SMILES: C=CC(=O)N1CCN(C2CN3c4cc(N5CCC(c6c(C)cnn6C6(OC)CC6)CC5)nc(C(F)F)c4C=CCC23)CC1]

or a pharmaceutically acceptable salt thereof. In some cases, the compound of Formula (II) is

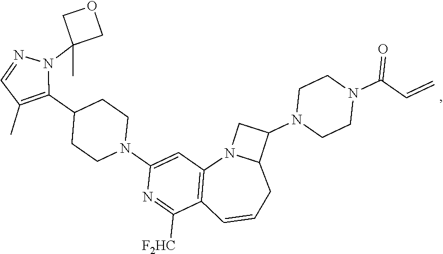 [SMILES: C=CC(=O)N1CCN(C2CN3c4cc(N5CCC(c6c(C)cnn6C6(C)COC6)CC5)nc(C(F)F)c4C=CCC23)CC1]

or a pharmaceutically acceptable salt thereof. In some cases, the compound of Formula (II) is

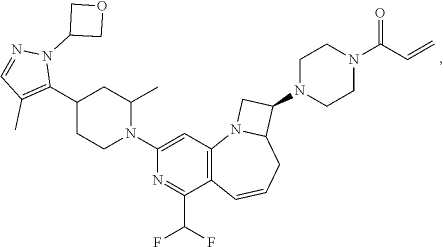 [SMILES: C=CC(=O)N1CCN([C@@H]2CN3c4cc(N5CCC(c6c(C)cnn6C6COC6)CC5C)nc(C(F)F)c4C=CCC23)CC1]

or a pharmaceutically acceptable salt thereof. In some cases, the compound of Formula (II) is

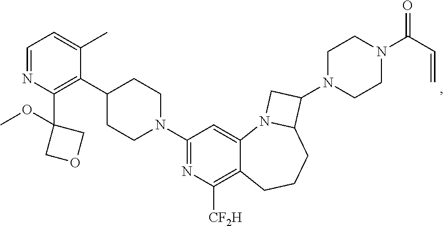 [SMILES: C=CC(=O)N1CCN(C2CN3c4cc(N5CCC(c6c(C)ccnc6C6(OC)COC6)CC5)nc(C(F)F)c4CCCC23)CC1]

or a pharmaceutically acceptable salt thereof. In some cases, the compound of Formula (II) is

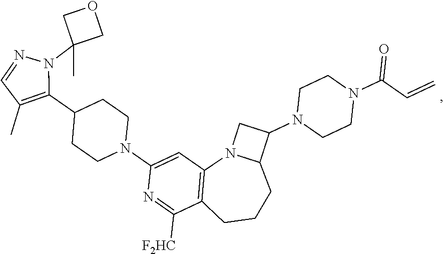 [SMILES: C=CC(=O)N1CCN(C2CN3c4cc(N5CCC(c6c(C)cnn6C6(C)COC6)CC5)nc(C(F)F)c4CCCC23)CC1]

or a pharmaceutically acceptable salt thereof. In some cases, the compound of Formula (II) is

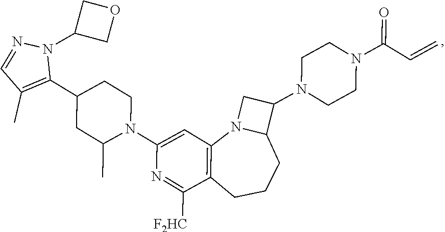 [SMILES: C=CC(=O)N1CCN(C2CN3c4cc(N5CCC(c6c(C)cnn6C6COC6)CC5C)nc(C(F)F)c4CCCC23)CC1]

or a pharmaceutically acceptable salt thereof. In some cases, the compound of Formula (II) is

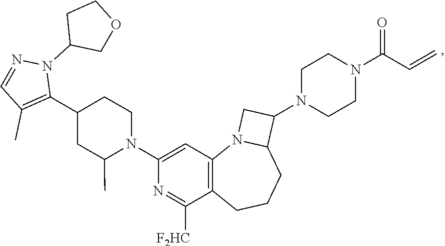 [SMILES: C=CC(=O)N1CCN(C2CN3c4cc(N5CCC(c6c(C)cnn6C6CCOC6)CC5C)nc(C(F)F)c4CCCC23)CC1]

or a pharmaceutically acceptable salt thereof. In some cases, the compound of Formula (II) is

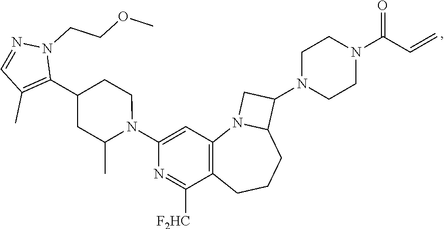 [SMILES: C=CC(=O)N1CCN(C2CN3c4cc(N5CCC(c6c(C)cnn6CCOC)CC5C)nc(C(F)F)c4CCCC23)CC1]

or a pharmaceutically acceptable salt thereof. In some cases, the compound of Formula (II) is

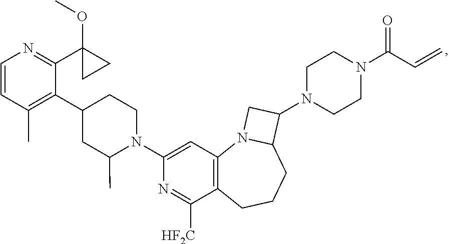

or a pharmaceutically acceptable salt thereof. In some cases, the compound of Formula (II) is

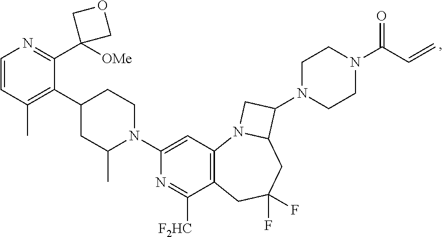 [SMILES: C=CC(=O)N1CCN(C2CN3c4cc(N5CCC(c6c(C)ccnc6C6(OC)COC6)CC5C)nc(C(F)F)c4CC(F)(F)CC23)CC1]

or a pharmaceutically acceptable salt thereof. In some cases, the compound of Formula (II) is

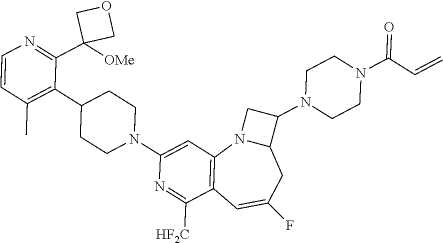

or a pharmaceutically acceptable salt thereof. In some cases, the compound of Formula (II) is

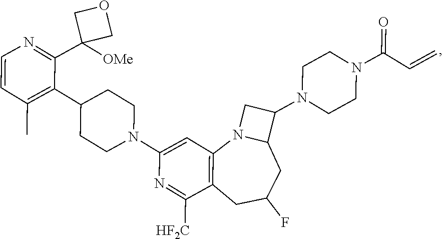

or pharmaceutically acceptable salt thereof. In some cases, the compound of Formula (II) is

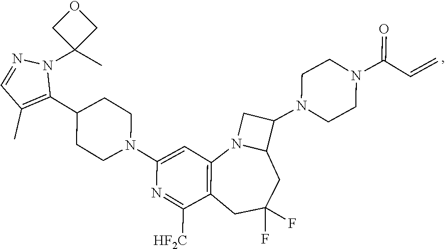

or a pharmaceutically acceptable salt thereof. In some cases, the compound of Formula (II) is

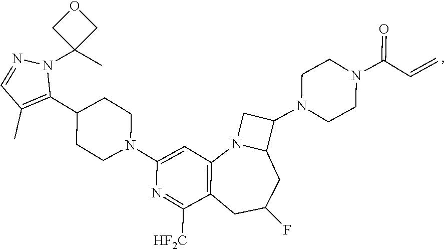

or a pharmaceutically acceptable salt thereof.

In some cases, the compound of Formula (II) is a compound listed in Table A′, below. If the stereochemistry of a structure or a portion of a structure in Table A′ is not explicitly shown (e.g., such as with dashed or bold lines), then the structure or portion of structure is either achiral or interpreted as being any of the possible stereoisomers of the structure or portion of the structure. In cases in which the stereochemistry of the structure or portion of the structure in Table A′ is explicitly shown, a single stereoisomer of the structure or portion of a structure is represented.

TABLE A

| Comp. | | |
|---|---|---|
| # | Chemical Structure | Name |
| 1-001 | 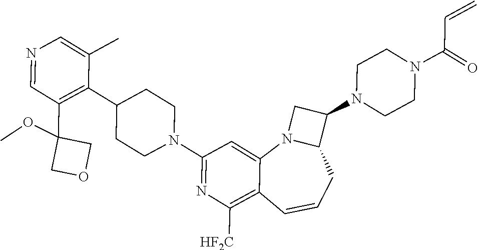 | 1-(4-((7aS,8R)-4-(difluoromethyl)-2-(4-(2-(3- methoxy-3-oxetanyl)-4-methyl-3-pyridinyl)-1- piperidinyl)-7,7a,8,9-tetrahydroazeto[1,2- a]pyrido[3,4-f]azepin-8-yl)-1-piperazinyl)-2- propen-1-one |
| 1-002 | 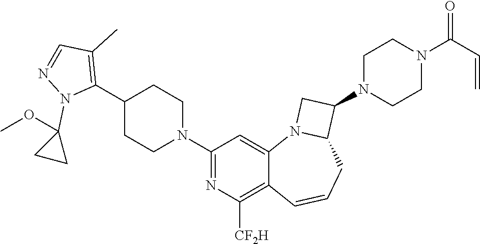 [SMILES: C=CC(=O)N1CCN([C@@H]2CN3c4cc(N5CCC(c6c(C)cnn6C6(OC)CC6)CC5)nc(C(F)F)c4C=CC[C@@H]23)CC1] | 1-(4-((7aS,8R)-4-(difluoromethyl)-2-(4-(1-(1- methoxycyclopropyl)-4-methyl-1H-pyrazol-5- yl)-1-piperidinyl)-7,7a,8,9-tetrahydroazeto[1,2- a]pyrido[3,4-f]azepin-8-yl)-1-piperazinyl)-2- propen-1-one |
| 1-003 | 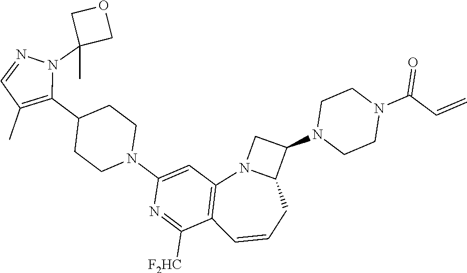 [SMILES: C=CC(=O)N1CCN([C@@H]2CN3c4cc(N5CCC(c6c(C)cnn6C6(C)COC6)CC5)nc(C(F)F)c4C=CC[C@@H]23)CC1] | 1-(4-((7aS,8R)-4-(difluoromethyl)-2-(4-(4- methyl-1-(3-methyl-3-oxetanyl)-1H-pyrazol-5- yl)-1-piperidinyl)-7,7a,8,9-tetrahydroazeto[1,2- a]pyrido[3,4-f]azepin-8-yl)-1-piperazinyl)-2- propen-1-one |
| 1-004 | 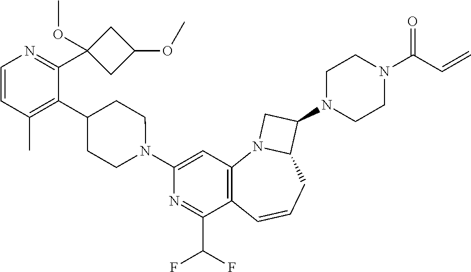 [SMILES: C=CC(=O)N1CCN([C@@H]2CN3c4cc(N5CCC(c6c(C)ccnc6C6(OC)CC(OC)C6)CC5)nc(C(F)F)c4C=CC[C@@H]23)CC1] | 1-(4-((7aS,8R)-4-(difluoromethyl)-2-(4-(2-(1,3- dimethoxycyclobutyl)-4-methylpyridin-3- yl)piperidin-1-yl)-7,7a,8,9-tetrahydroazeto[1,2- a]pyrido[3,4-f]azepin-8-yl)piperazin-1-yl)prop- 2-en-1-one |
| 1-005 | 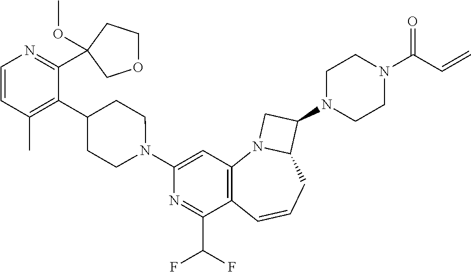 [SMILES: C=CC(=O)N1CCN([C@@H]2CN3c4cc(N5CCC(c6c(C)ccnc6C6(OC)CCOC6)CC5)nc(C(F)F)c4C=CC[C@@H]23)CC1] | 1-(4-((7aS,8R)-4-(difluoromethyl)-2-(4-(2-(3- methoxytetrahydro-3-furanyl)-4-methyl-3- pyridinyl)-1-piperidinyl)-7,7a,8,9- tetrahydroazeto[1,2-a]pyrido[3,4-f]azepin-8-yl)- 1-piperazinyl)-2-propen-1-one |
| 1-006 | 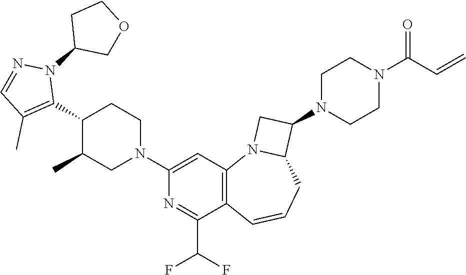 [SMILES: C=CC(=O)N1CCN([C@@H]2CN3c4cc(N5CC[C@@H](c6c(C)cnn6[C@H]6CCOC6)[C@H](C)C5)nc(C(F)F)c4C=CC[C@@H]23)CC1] | 1-(4-((7aS,8R)-4-(difluoromethyl)-2-((3S,4R)- 3-methyl-4-(4-methyl-1-((3S)-tetrahydro-3- furanyl)-1H-pyrazol-5-yl)-1-piperidinyl)- 7,7a,8,9-tetrahydroazeto[1,2-a]pyrido[3,4- f]azepin-8-yl)-1-piperazinyl)-2-propen-1-one |
| 1-007 | 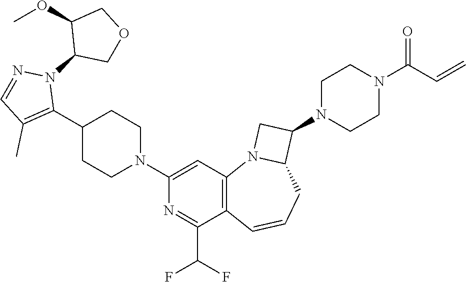 [SMILES: C=CC(=O)N1CCN([C@@H]2CN3c4cc(N5CCC(c6c(C)cnn6[C@@H]6COC[C@@H]6OC)CC5)nc(C(F)F)c4C=CC[C@@H]23)CC1] | 1-(4-((7aS,8R)-4-(difluoromethyl)-2-(4-(1- ((3R,4R)-4-methoxytetrahydro-3-furanyl)-4- methyl-1H-pyrazol-5-yl)-1-piperidinyl)- 7,7a,8,9-tetrahydroazeto[1,2-a]pyrido[3,4- f]azepin-8-yl)-1-piperazinyl)-2-propen-1-one |
| 1-008 | 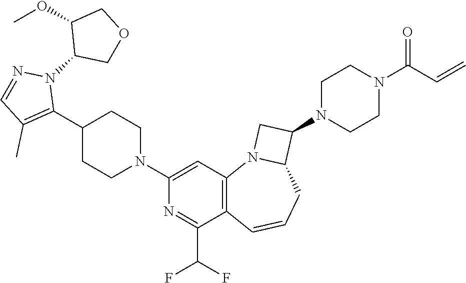 [SMILES: C=CC(=O)N1CCN([C@@H]2CN3c4cc(N5CCC(c6c(C)cnn6[C@H]6COC[C@H]6OC)CC5)nc(C(F)F)c4C=CC[C@@H]23)CC1] | 1-(4-((7aS,8R)-4-(difluoromethyl)-2-(4-(1- ((3S,4S)-4-methoxytetrahydro-3-furanyl)-4- methyl-1H-pyrazol-5-yl)-1-piperidinyl)- 7,7a,8,9-tetrahydroazeto[1,2-a]pyrido[3,4- f]azepin-8-yl)-1-piperazinyl)-2-propen-1-one |
| 1-009 | 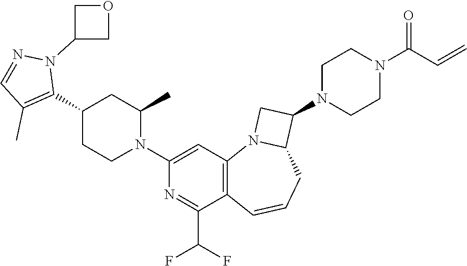 [SMILES: C=CC(=O)N1CCN([C@@H]2CN3c4cc(N5CC[C@H](c6c(C)cnn6C6COC6)C[C@H]5C)nc(C(F)F)c4C=CC[C@@H]23)CC1] | 1-(4-((7aS,8R)-4-(difluoromethyl)-2-((2R,4S)- 2-methyl-4-(4-methyl-1-(3-oxetanyl)-1H- pyrazol-5-yl)-1-piperidinyl)-7,7a,8,9- tetrahydroazeto[1,2-a]pyrido[3,4-f]azepin-8-yl)- 1-piperazinyl)-2-propen-1-one |
| 1-010 | 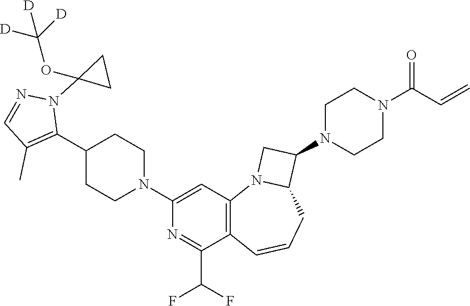 [SMILES: [2H]C([2H])([2H])OC1(n2ncc(C)c2C2CCN(c3cc4c(c(C(F)F)n3)C=CC[C@H]3[C@H](N5CCN(C(=O)C=C)CC5)CN43)CC2)CC1] | 1-(4-((7aS,8R)-4-(difluoromethyl)-2-(4-(1-(1- (methoxy-d3)cyclopropyl)-4-methyl-1H- pyrazol-5-yl)piperidin-1-yl)-7,7a,8,9- tetrahydroazeto[1,2-a]pyrido[3,4-f]azepin-8- yl)piperazin-1-yl)prop-2-en-1-one |
| 1-011 | 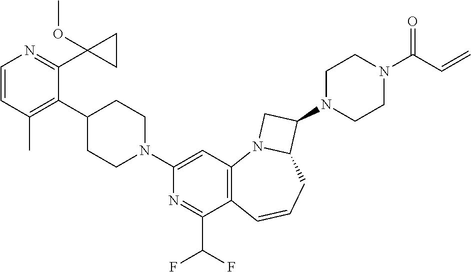 [SMILES: C=CC(=O)N1CCN([C@@H]2CN3c4cc(N5CCC(c6c(C)ccnc6C6(OC)CC6)CC5)nc(C(F)F)c4C=CC[C@@H]23)CC1] | 1-(4-((7aS,8R)-4-(difluoromethyl)-2-(4-(2-(1- methoxycyclopropyl)-4-methyl-3-pyridinyl)-1- piperidinyl)-7,7a,8,9-tetrahydroazeto[1,2- a]pyrido[3,4-f]azepin-8-yl)-1-piperazinyl)-2- propen-1-one |
| 1-012 | 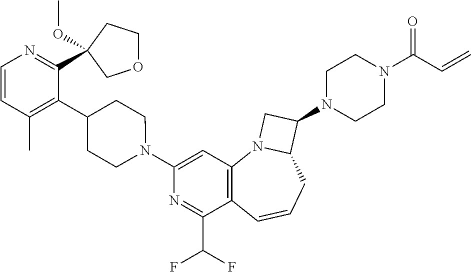 [SMILES: C=CC(=O)N1CCN([C@@H]2CN3c4cc(N5CCC(c6c(C)ccnc6[C@@]6(OC)CCOC6)CC5)nc(C(F)F)c4C=CC[C@@H]23)CC1] | 1-(4-((7aS,8R)-4-(difluoromethyl)-2-(4-(2-((S)- 3-methoxytetrahydrofuran-3-yl)-4- methylpyridin-3-yl)piperidin-1-yl)-7,7a,8,9- tetrahydroazeto[1,2-a]pyrido[3,4-f]azepin-8- yl)piperazin-1-yl)prop-2-en-1-one |
| 1-013 | 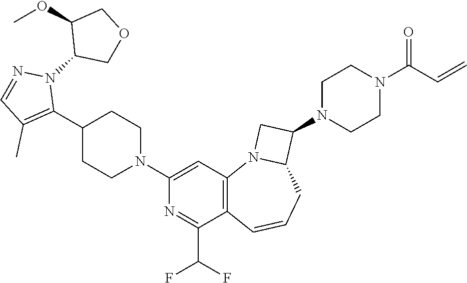 [SMILES: C=CC(=O)N1CCN([C@@H]2CN3c4cc(N5CCC(c6c(C)cnn6[C@H]6COC[C@@H]6OC)CC5)nc(C(F)F)c4C=CC[C@@H]23)CC1] | 1-(4-((7aS,8R)-4-(difluoromothyl)-2-(4-(1- ((3S,4R)-4-methoxytetrahydro-3-furanyl)-4- methyl-1H-pyrazol-5-yl)-1-piperidinyl)- 7,7a,8,9-tetrahydroazeto[1,2-a]pyrido[3,4- f]azepin-8-yl)-1-piperazinyl)-2-propen-1-one |
| 1-014 | 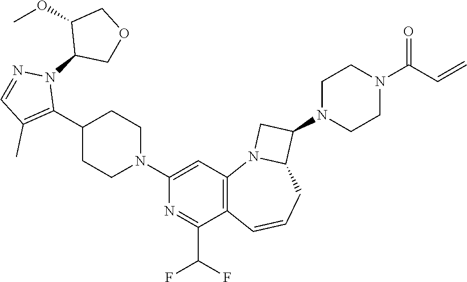 [SMILES: C=CC(=O)N1CCN([C@@H]2CN3c4cc(N5CCC(c6c(C)cnn6[C@@H]6COC[C@H]6OC)CC5)nc(C(F)F)c4C=CC[C@@H]23)CC1] | 1-(4-((7aS,8R)-4-(difluoromethyl)-2-(4-(1- ((3R,4S)-4-methoxytetrahydro-3-furanyl)-4- methyl-1H-pyrazol-5-yl)-1-piperidinyl)- 7,7a,8,9-tetrahydroazeto[1,2-a]pyrido[3,4- f]azepin-8-yl)-1-piperazinyl)-2-propen-1-one |
| 1-015 | 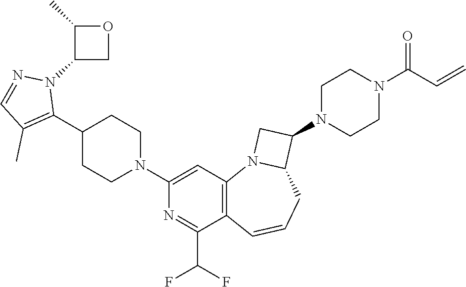 [SMILES: C=CC(=O)N1CCN([C@@H]2CN3c4cc(N5CCC(c6c(C)cnn6[C@H]6CO[C@H]6C)CC5)nc(C(F)F)c4C=CC[C@@H]23)CC1] | 1-(4-((7aS,8R)-4-(difluoromethyl)-2-(4-(4- methyl-1-((25,3S)-2-methyl-3-oxetanyl)-1H- pyrazol-5-yl)-1-piperidinyl)-7,7a,8,9- tetrahydroazeto[1,2-a]pyrido[3,4-f]azepin-8-yl)- 1-piperazinyl)-2-propen-1-one |
| 1-016 | 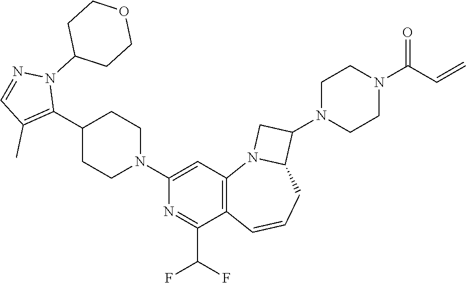 [SMILES: C=CC(=O)N1CCN(C2CN3c4cc(N5CCC(c6c(C)cnn6C6CCOCC6)CC5)nc(C(F)F)c4C=CC[C@@H]23)CC1] | 1-(4-((7aS,8R)-4-(difluoromethyl)-2-(4-(4- methyl-1-(tetrahydro-2H-pyran-4-yl)-1H- pyrazol-5-yl)-1-piperidinyl)-7,7a,8,9- tetrahydroazeto[1,2-a]pyrido[3,4-f]azepin-8-yl)- 1-piperazinyl)-2-propen-1-one |
| 1-017 | 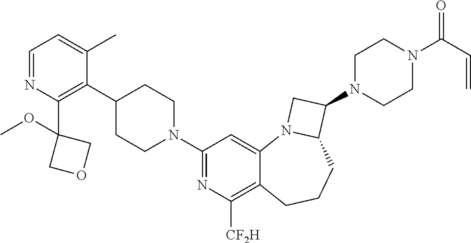 [SMILES: C=CC(=O)N1CCN([C@@H]2CN3c4cc(N5CCC(c6c(C)ccnc6C6(OC)COC6)CC5)nc(C(F)F)c4CCC[C@@H]23)CC1] | 1-(4-((7aS,8R)-4-(difluoromethyl)-2-(4-(2-(3- methoxy-3-oxetanyl)-4-methyl-3-pyridinyl)-1- piperidinyl)-5,6,7,7a,8,9-hexahydroazeto[1,2- a]pyrido[3,4-f]azepin-8-yl)-1-piperazinyl)-2- propen-1-one |
| 1-018 | 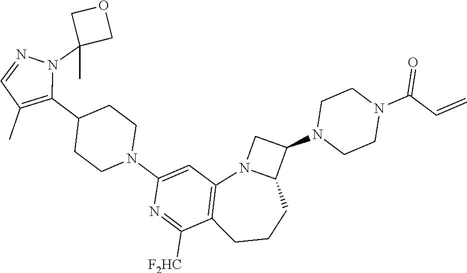 [SMILES: C=CC(=O)N1CCN([C@@H]2CN3c4cc(N5CCC(c6c(C)cnn6C6(C)COC6)CC5)nc(C(F)F)c4CCC[C@@H]23)CC1] | 1-(4-((7aS,8R)-4-(difluoromethyl)-2-(4-(4- methyl-1-(3-methyloxetan-3-yl)-1H-pyrazol-5- yl)piperidin-1-yl)-5,6,7,7a,8,9- hexahydroazeto[1,2-a]pyrido[3,4-f]azepin-8- yl)piperazin-1-yl)prop-2-en-1-one |
| 1-019 | 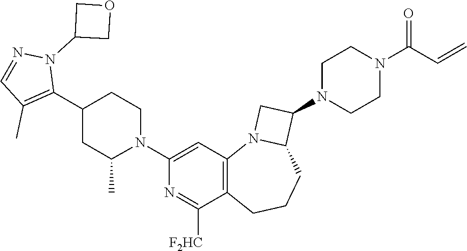 [SMILES: C=CC(=O)N1CCN([C@@H]2CN3c4cc(N5CCC(c6c(C)cnn6C6COC6)C[C@H]5C)nc(C(F)F)c4CCC[C@@H]23)CC1] | 1-(4-((7aS,8R)-4-(difluoromethyl)-2-((2R,4S)- 2-methyl-4-(4-methyl-1-(3-oxetanyl)-1H- pyrazol-5-yl)-1-piperidinyl)-5,6,7,7a,8,9- hexahydroazeto[1,2-a]pyrido[3,4-f]azepin-8-yl)- 1-piperazinyl)-2-propen-1-one |
| 1-020 | 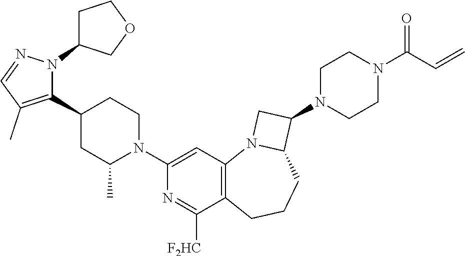 [SMILES: C=CC(=O)N1CCN([C@@H]2CN3c4cc(N5CC[C@H](c6c(C)cnn6[C@H]6CCOC6)C[C@H]5C)nc(C(F)F)c4CCC[C@@H]23)CC1] | 1-(4-((7aS,8R)-4-(difluoromethyl)-2-((2R,4S)- 2-methyl-4-(4-methyl-1-((3S)-tetrahydro-3- furanyl)-1H-pyrazol-5-yl)-1-piperidinyl)- 5,6,7,7a,8,9-hexahydroazeto[1,2-a]pyrido[3,4- f]azepin-8-yl)-1-piperazinyl)-2-propen-1-one |
| 1-021 | 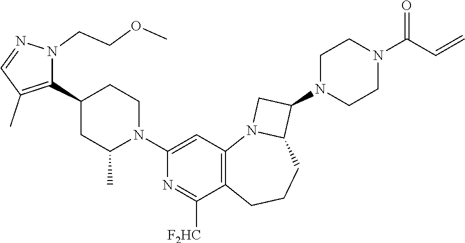 [SMILES: C=CC(=O)N1CCN([C@@H]2CN3c4cc(N5CC[C@H](c6c(C)cnn6CCOC)C[C@H]5C)nc(C(F)F)c4CCC[C@@H]23)CC1] | 1-(4-((7aS,8R)-4-(difluoromethyl)-2-((2R,4S)- 4-(1-(2-methoxyethyl)-4-methyl-1H-pyrazol-5- yl)-2-methyl-1-piperidinyl)-5,6,7,7a,8,9- hexahydroazeto[1,2-a]pyrido[3,4-f]azepin-8-yl)- 1-piperazinyl)-2-propen-1-one |
| 1-022 | 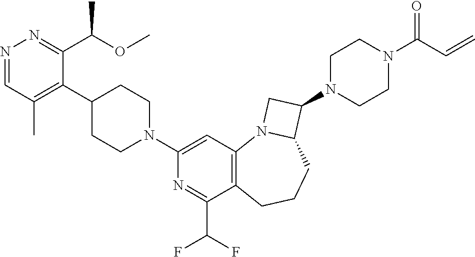 [SMILES: C=CC(=O)N1CCN([C@@H]2CN3c4cc(N5CCC(c6c(C)cnnc6[C@@H](C)OC)CC5)nc(C(F)F)c4CCC[C@@H]23)CC1] | 1-(4-((7aS,8R)-4-(difluoromethyl)-2-(4-(3- ((1R)-1-methoxyethyl)-5-methyl-4- pyridazinyl)-1-piperidinyl)-5,6,7,7a,8,9- hexahydroazeto[1,2-a]pyrido[3,4-f]azepin-8-yl)- 1-piperazinyl)-2-propen-1-one |
| 1-023 | 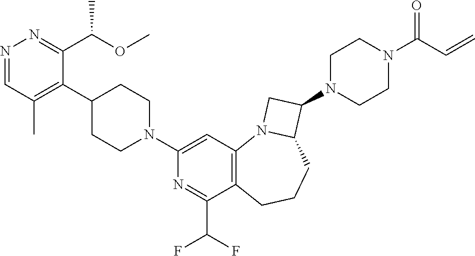 [SMILES: C=CC(=O)N1CCN([C@@H]2CN3c4cc(N5CCC(c6c(C)cnnc6[C@H](C)OC)CC5)nc(C(F)F)c4CCC[C@@H]23)CC1] | 1-(4-((7aS,8R)-4-(difluoromethyl)-2-(4-(3- ((1S)-1-methoxyethyl)-5-methyl-4-pyridazinyl)- 1-piperidinyl)-5,6,7,7a,8,9-hexahydroazeto[1,2- a]pyrido[3,4-f]azepin-8-yl)-1-piperazinyl)-2- propen-1-one |
| 1-024 | 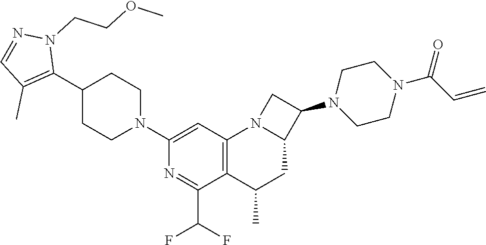 [SMILES: C=CC(=O)N1CCN([C@@H]2CN3c4cc(N5CCC(c6c(C)cnn6CCOC)CC5)nc(C(F)F)c4[C@@H](C)C[C@@H]23)CC1] | 1-(4-((5S,6aS,7R)-4-(difluoromethyl)-2-(4-(1- (2-methoxyethyl)-4-methyl-1H-pyrazol-5-yl)-1- piperidinyl)-5-methyl-6,6a,7,8-tetrahydro-5H- azeto[1,2-a][1,6]naphthyridin-7-yl)-1- piperazinyl)-2-propen-1-one |
| 1-025 | 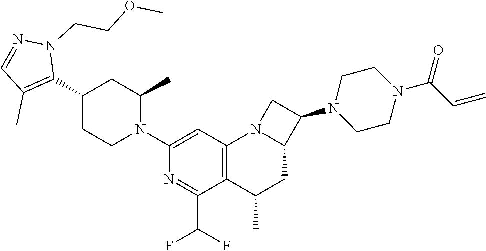 [SMILES: C=CC(=O)N1CCN([C@@H]2CN3c4cc(N5CC[C@H](c6c(C)cnn6CCOC)C[C@H]5C)nc(C(F)F)c4[C@@H](C)C[C@@H]23)CC1] | 1-(4-((5S,6aS,7R)-4-(difluoromethyl)-2- ((2R,4S)-4-(1-(2-methoxyethyl)-4-methyl-1H- pyrazol-5-yl)-2-methyl-1-piperidinyl)-5-methyl- 6,6a,7,8-tetrahydro-5H-azeto[1,2- a][1,6]naphthyridin-7-yl)-1-piperazinyl)-2- propen-1-one |
| 1-026 | 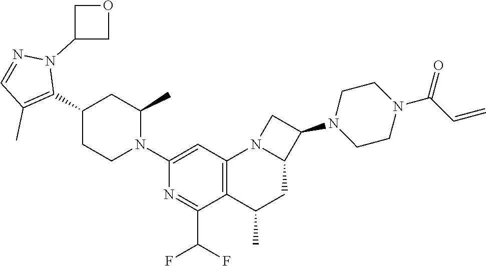 [SMILES: C=CC(=O)N1CCN([C@@H]2CN3c4cc(N5CC[C@H](c6c(C)cnn6C6COC6)C[C@H]5C)nc(C(F)F)c4[C@@H](C)C[C@@H]23)CC1] | 1-(4-((5S,6aS,7R)-4-(difluoromethyl)-5-methyl- 2-((2R,4S)-2-methyl-4-(4-methyl-1-(3- oxetanyl)-1H-pyrazol-5-yl)-1-piperidinyl)- 6,6a,7,8-tetrahydro-5H-azeto[1,2- a][1,6]naphthyridin-7-yl)-1-piperazinyl)-2- propen-1-one |
| 1-027 | 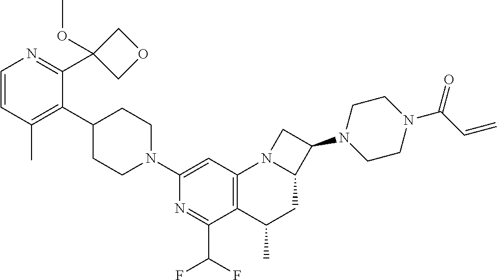 [SMILES: C=CC(=O)N1CCN([C@@H]2CN3c4cc(N5CCC(c6c(C)ccnc6C6(OC)COC6)CC5)nc(C(F)F)c4[C@@H](C)C[C@@H]23)CC1] | 1-(4-((5S,6aS,7R)-4-(difluoromethyl)-2-(4-(2- (3-methoxy-3-oxetanyl)-4-methyl-3-pyridinyl)- 1-piperidinyl)-5-methyl-6,6a,7,8-tetrahydro-5H- azeto[1,2-a][1,6]naphthyridin-7-yl)-1- piperazinyl)-2-propen-1-one |
| 1-028 | 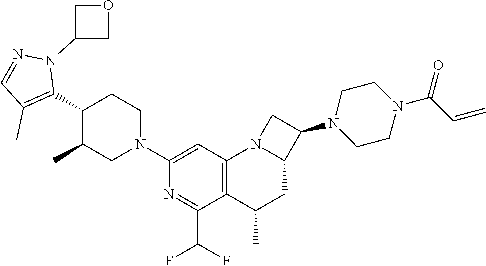 [SMILES: C=CC(=O)N1CCN([C@@H]2CN3c4cc(N5CC[C@@H](c6c(C)cnn6C6COC6)[C@H](C)C5)nc(C(F)F)c4[C@@H](C)C[C@@H]23)CC1] | 1-(4-((5S,6aS,7R)-4-(difluoromethyl)-5-methyl- 2-((3S,4R)-3-methyl-4-(4-methyl-1-(3- oxetanyl)-1H-pyrazol-5-yl)-1-piperidinyl)- 6,6a,7,8-tetrahydro-5H-azeto[1,2- a][1,6]naphthyridin-7-yl)-1-piperazinyl)-2- propen-1-one |
| 1-029 | 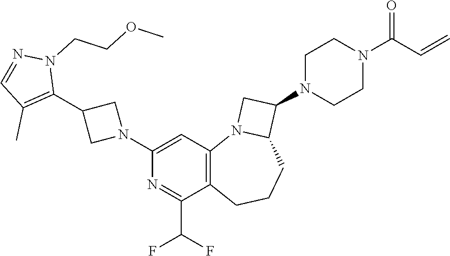 [SMILES: C=CC(=O)N1CCN([C@@H]2CN3c4cc(N5CC(c6c(C)cnn6CCOC)C5)nc(C(F)F)c4CCC[C@@H]23)CC1] | 1-(4-((7aS,8R)-4-(difluoromethyl)-2-(4-(1-(2- methoxyethyl)-4-methyl-1H-pyrazol-5-yl)-1- piperidinyl)-5,6,7,7a,8,9-hexahydroazeto[1,2- a]pyrido[3,4-f]azepin-8-yl)-1-piperazinyl)-2- propen-1-one |
| 1-030 | 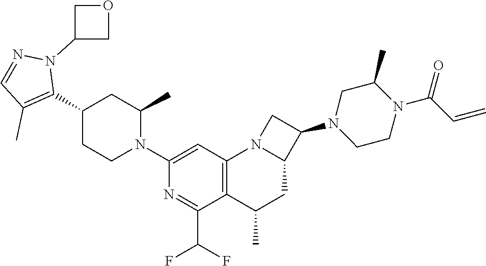 [SMILES: C=CC(=O)N1CCN([C@@H]2CN3c4cc(N5CC[C@H](c6c(C)cnn6C6COC6)C[C@H]5C)nc(C(F)F)c4[C@@H](C)C[C@@H]23)C[C@H]1C] | 1-((2R)-4-((58,6aS,7R)-4-(difluoromethyl)-5- methyl-2-((2R,4S)-2-methyl-4-(4-methyl-1-(3- oxetanyl)-1H-pyrazol-5-yl)-1-piperidinyl)- 6,6a,7,8-tetrahydro-5H-azeto[1,2- a][1,6]naphthyridin-7-yl)-2-methyl-1- piperazinyl)-2-propen-1-one |
| 1-031 | 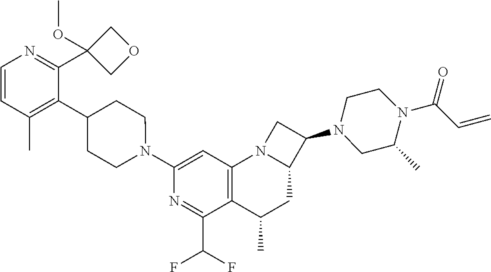 [SMILES: C=CC(=O)N1CCN([C@@H]2CN3c4cc(N5CCC(c6c(C)ccnc6C6(OC)COC6)CC5)nc(C(F)F)c4[C@@H](C)C[C@@H]23)C[C@H]1C] | 1-((2R)-4-((5S,6aS,7R)-4-(difluoromethyl)-2- (4-(2-(3-methoxy-3-oxetanyl)-4-methyl-3- pyridinyl)-1-piperidinyl)-5-methyl-6,6a,7,8- tetrahydro-5H-azeto[1,2-a][1,6]naphthyridin-7- yl)-2-methyl-1-piperazinyl)-2-propen-1-one |
| 1-032 | 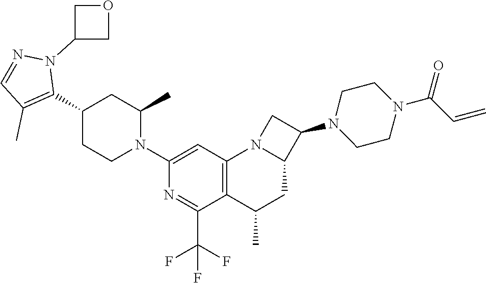 [SMILES: C=CC(=O)N1CCN([C@@H]2CN3c4cc(N5CC[C@H](c6c(C)cnn6C6COC6)C[C@H]5C)nc(C(F)(F)F)c4[C@@H](C)C[C@@H]23)CC1] | 1-(4-((5S,6aS,7R)-5-methyl-2-((2R,4S)-2- methyl-4-(4-methyl-1-(3-oxetany)-1H-pyrazol- 5-yl)-1-piperidinyl)-4-(trifluoromethyl)- 6,6a,7,8-tetrahydro-5H-azeto[1,2- a][1,6]naphthyridin-7-yl)-1-piperazinyl)-2- propen-1-one |
| 1-033 | 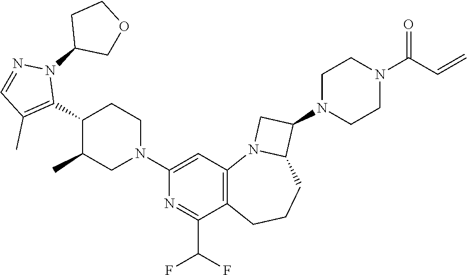 [SMILES: C=CC(=O)N1CCN([C@@H]2CN3c4cc(N5CC[C@@H](c6c(C)cnn6[C@H]6CCOC6)[C@H](C)C5)nc(C(F)F)c4CCC[C@@H]23)CC1] | 1-(4-((7aS,8R)-4-(difluoromethyl)-2-((3S,4R)- 3-methyl-4-(4-methyl-1-((3S)-tetrahydro-3- furanyl)-1H-pyrazol-5-yl)-1-piperidinyl)- 5,6,7,7a,8,9-hexahydroazeto[1,2-a]pyrido[3,4- f]azepin-8-yl)-1-piperazinyl)-2-propen-1-one |
| 1-034 | 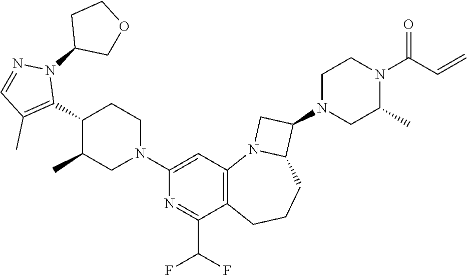 [SMILES: C=CC(=O)N1CCN([C@@H]2CN3c4cc(N5CC[C@@H](c6c(C)cnn6[C@H]6CCOC6)[C@H](C)C5)nc(C(F)F)c4CCC[C@@H]23)C[C@H]1C] | 1-((2R)-4-((7aS,8R)-4-(difluoromethyl)-2- ((35,4R)-3-methyl-4-(4-methyl-1-((3S)- tetrahydro-3-furanyl)-1H-pyrazol-5-yl)-1- piperidinyl)-5,6,7,7a,8,9-hexahydroazeto[1,2- a]pyrido[3,4-f]azepin-8-yl)-2-methyl-1- piperazinyl)-2-propen-1-one |
| 1-035 | 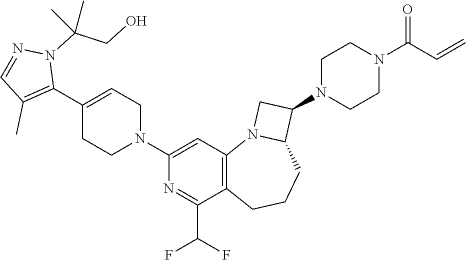 [SMILES: C=CC(=O)N1CCN([C@@H]2CN3c4cc(N5CC=C(c6c(C)cnn6C(C)(C)CO)CC5)nc(C(F)F)c4CCC[C@@H]23)CC1] | 1-(4-((7aS,8R)-4-(difluoromethyl)-2-(4-(1-(1- hydroxy-2-methyl-2-propanyl)-4-methyl-1H- pyrazol-5-yl)-3,6-dihydro-1(2H)-pyridinyl)- 5,6,7,7a,8,9-hexahydroazeto[1,2-a]pyrido[3,4- f]azepin-8-yl)-1-piperazinyl)-2-propen-1-one |
| 1-036 | 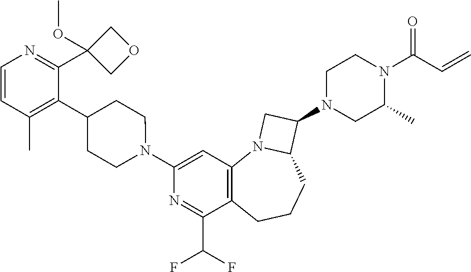 [SMILES: C=CC(=O)N1CCN([C@@H]2CN3c4cc(N5CCC(c6c(C)ccnc6C6(OC)COC6)CC5)nc(C(F)F)c4CCC[C@@H]23)C[C@H]1C] | 1-((2R)-4-((7aS,8R)-4-(difluoromethyl)-2-(4-(2- (3-methoxy-3-oxetanyl)-4-methyl-3-pyridinyl)- 1-piperidinyl)-5,6,7,7a,8,9-hexahydroazeto[1,2- a]pyrido[3,4-f]azepin-8-yl)-2-methyl-1- piperazinyl)-2-propen-1-one |
| 1-037 | 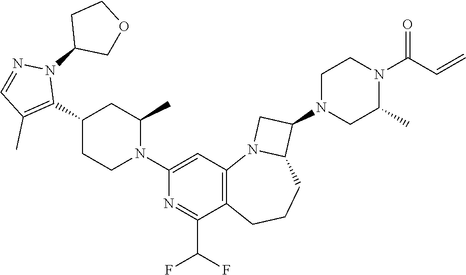 [SMILES: C=CC(=O)N1CCN([C@@H]2CN3c4cc(N5CC[C@H](c6c(C)cnn6[C@H]6CCOC6)C[C@H]5C)nc(C(F)F)c4CCC[C@@H]23)C[C@H]1C] | 1-((2R)-4-((7aS,8R)-4-(difluoromethyl)-2- (2R,4S)-2-methyl-4-(4-methyl-1-(3S)- tetrahydro-3-furanyl)-1H-pyrazol-5-yl)-1- piperidinyl)-5,6,7,7a,8,9-hexahydroazeto[1,2- a]pyrido[3,4-f]azepin-8-yl)-2-methyl-1- piperazinyl)-2-propen-1-one |
| 1-038 | 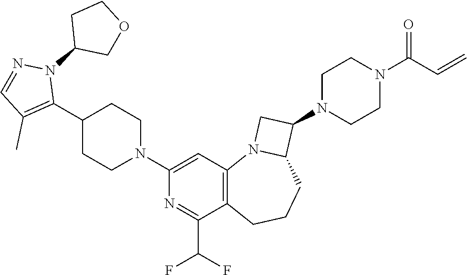 [SMILES: C=CC(=O)N1CCN([C@@H]2CN3c4cc(N5CCC(c6c(C)cnn6[C@H]6CCOC6)CC5)nc(C(F)F)c4CCC[C@@H]23)CC1] | 1-(4-((7aS,8R)-4-(difluoromethyl)-2-(4-(4- methyl-1-((3S)-tetrahydro-3-furanyl)-1H- pyrazol-5-yl)-1-piperidinyl)-5,6,7,7a,8,9- hexahydroazeto[1,2-a]pyrido[3,4-f]azepin-8-yl)- 1-piperazinyl)-2-propen-1-one |
| 1-039 | 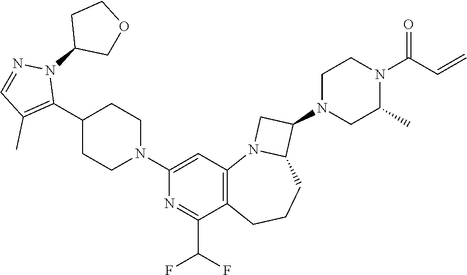 [SMILES: C=CC(=O)N1CCN([C@@H]2CN3c4cc(N5CCC(c6c(C)cnn6[C@H]6CCOC6)CC5)nc(C(F)F)c4CCC[C@@H]23)C[C@H]1C] | 1-((2R)-4-((7aS,8R)-4-(difluoromethyl)-2-(4-(4- methyl-1-((3S)-tetrahydro-3-furanyl)-1H- pyrazol-5-yl)-1-piperidinyl)-5,6,7,7a,8,9- hexahydroazeto[1,2-a]pyrido[3,4-f]azepin-8-yl)- 2-methyl-1-piperazinyl)-2-propen-1-one |
| 1-040 | 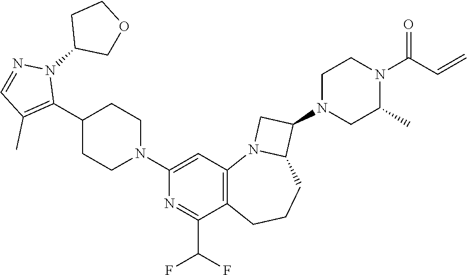 [SMILES: C=CC(=O)N1CCN([C@@H]2CN3c4cc(N5CCC(c6c(C)cnn6[C@@H]6CCOC6)CC5)nc(C(F)F)c4CCC[C@@H]23)C[C@H]1C] | 1-(4-(7aS,8R)-4-(difluoromethyl)-2-(4-(4- methyl-1-((3R)-tetrahydro-3-furanyl)-1H- pyrazol-5-yl)-1-piperidinyl)-5,6,7,7a,8,9- hexahydroazeto[1,2-a]pyrido[3,4-f]azepin-8-yl)- 1-piperazinyl)-2-propen-1-one |
| 1-041 | 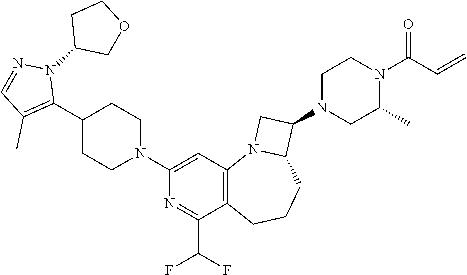 [SMILES: C=CC(=O)N1CCN([C@@H]2CN3c4cc(N5CCC(c6c(C)cnn6[C@@H]6CCOC6)CC5)nc(C(F)F)c4CCC[C@@H]23)C[C@H]1C] | 1-((2R)-4-((7aS,8R)-4-(difluoromethyl)-2-(4-(4- methyl-1-((3R)-tetrahydro-3-furanyl)-1H- pyrazol-5-yl)-1-piperidinyl)-5,6,7,7a,8,9- hexahydroazeto[1,2-a]pyrido[3,4-f]azepin-8-yl)- 2-methyl-1-piperaziny])-2-propen-1-one |
| 1-042 | 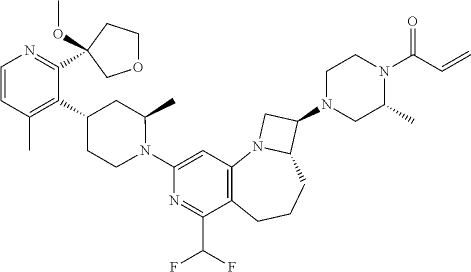 [SMILES: C=CC(=O)N1CCN([C@@H]2CN3c4cc(N5CC[C@H](c6c(C)ccnc6[C@]6(OC)CCOC6)C[C@H]5C)nc(C(F)F)c4CCC[C@@H]23)C[C@H]1C] | 1-(4-((7aS,8R)-4-(difluoromethyl)-2-((2R,4S)- 4-(2-((3R)-3-methoxytetrahydro-3-furanyl)-4- methyl-3-pyridinyl)-2-methyl-1-piperidinyl)- 5,6,7,7a,8,9-hexahydroazeto[1,2-a]pyrido[3,4- f]azepin-8-yl)-1-piperazinyl)-2-propen-1-one |
| 1-043 | 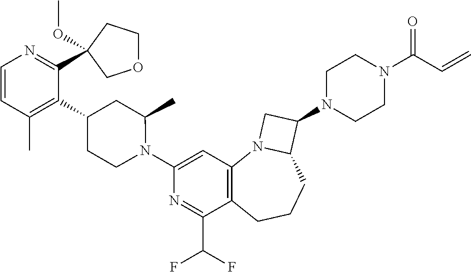 [SMILES: C=CC(=O)N1CCN([C@@H]2CN3c4cc(N5CC[C@H](c6c(C)ccnc6[C@@]6(OC)CCOC6)C[C@H]5C)nc(C(F)F)c4CCC[C@@H]23)CC1] | 1-(4-((7aS,8R)-4-(difluoromethyl)-2-((2R,4S)- 4-(2-((3S)-3-methoxytetrahydro-3-furanyl)-4- methyl-3-pyridinyl)-2-methyl-1-piperidinyl)- 5,6,7,7a,8,9-hexahydroazeto[1,2-a]pyrido[3,4- f]azepin-8-yl)-1-piperazinyl)-2-propen-1-one |
| 1-044 | 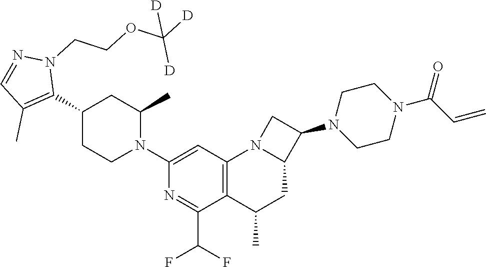 [SMILES: [2H]C([2H])([2H])OCCn1ncc(C)c1[C@H]1CCN(c2cc3c(c(C(F)F)n2)[C@@H](C)C[C@H]2[C@H](N4CCN(C(=O)C=C)CC4)CN32)[C@H](C)C1] | 1-(4-((5S,6aS,7R)-4-(difluoromethyl)-2- ((2R,4S)-4-(1-(2-(methoxy-d3)ethyl)-4-methyl- 1H-pyrazol-5-yl)-2-methylpiperidin-1-yl)-5- methyl-6,6a,7,8-tetrahydro-5H-azeto[1,2- a][1,6]naphthyridin-7-yl)piperazin-1-yl)prop-2- en-1-one |
| 1-045 | 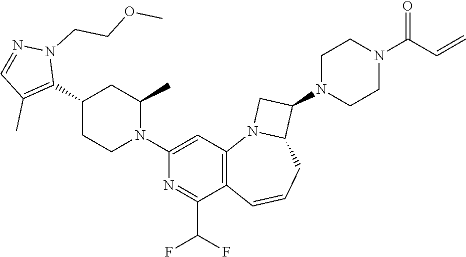 [SMILES: C=CC(=O)N1CCN([C@@H]2CN3c4cc(N5CC[C@H](c6c(C)cnn6CCOC)C[C@H]5C)nc(C(F)F)c4C=CC[C@@H]23)CC1] | 1-(4-((7aS,8R)-4-(difluoromethyl)-2-((2R,4S)- 4-(1-(2-methoxyethyl)-4-methyl-1H-pyrazol-5- yl)-2-methyl-1-piperidinyl)-7,7a,8,9- tetrahydroazeto[1,2-a]pyrido[3,4-f]azepin-8-yl)- 1-piperazinyl)-2-propen-1-one |
| 1-046 | 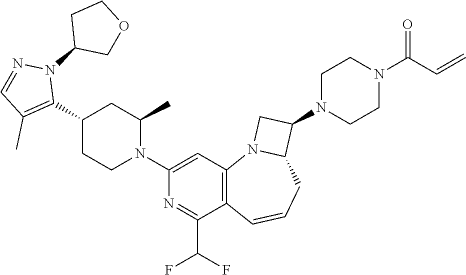 [SMILES: C=CC(=O)N1CCN([C@@H]2CN3c4cc(N5CC[C@H](c6c(C)cnn6[C@H]6CCOC6)C[C@H]5C)nc(C(F)F)c4C=CC[C@@H]23)CC1] | 1-(4-((7aS,8R)-4-(difluoromethyl)-2-((2R,4S)- 2-methyl-4-(4-methyl-1-((3S)-tetrahydro-3- furanyl)-1H-pyrazol-5-yl)-1-piperidinyl)- 7,7a,8,9-tetrahydroazeto[1,2-a]pyrido[3,4- f]azepin-8-yl)-1-piperazinyl)-2-propen-1-one |
| 1-047 | 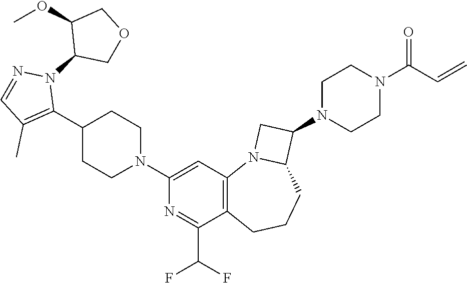 [SMILES: C=CC(=O)N1CCN([C@@H]2CN3c4cc(N5CCC(c6c(C)cnn6[C@@H]6COC[C@@H]6OC)CC5)nc(C(F)F)c4CCC[C@@H]23)CC1] | 1-(4-((7aS,8R)-4-(difluoromethyl)-2-(4-(1- ((3R,4R)-4-methoxytetrahydro-3-furanyl)-4- methyl-1H-pyrazol-5-yl)-1-piperidinyl)- 5,6,7,7a,8,9-hexahydroazeto[1,2-a]pyrido[3,4- f]azepin-8-yl)-1-piperazinyl)-2-propen-1-one |
| 1-048 | 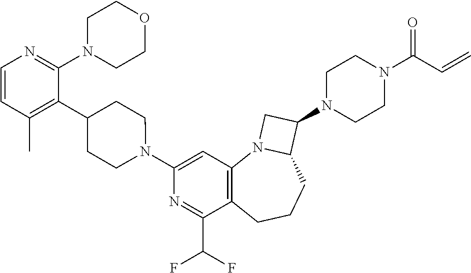 [SMILES: C=CC(=O)N1CCN([C@@H]2CN3c4cc(N5CCC(c6c(C)ccnc6N6CCOCC6)CC5)nc(C(F)F)c4CCC[C@@H]23)CC1] | 1-(4-((7aS,8R)-4-(difluoromethyl)-2-(4-(4- methyl-2-(4-morpholinyl)-3-pyridinyl)-1- piperidinyl)-5,6,7,7a,8,9-hexahydroazeto[1,2- a]pyrido[3,4-f]azepin-8-yl)-1-piperazinyl)-2- propen-1-one |
| 1-049 | 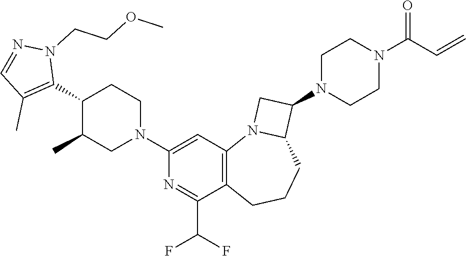 [SMILES: C=CC(=O)N1CCN([C@@H]2CN3c4cc(N5CC[C@@H](c6c(C)cnn6CCOC)[C@H](C)C5)nc(C(F)F)c4CCC[C@@H]23)CC1] | 1-(4-((7aS,8R)-4-(difluoromethyl)-2-((3S,4R)- 4-(1-(2-methoxyethyl)-4-methyl-1H-pyrazol-5- yl)-3-methyl-1-piperidinyl)-5,6,7,7a,8,9- hexahydroazeto[1,2-a]pyrido[3,4-f]azepin-8-yl)- 1-piperazinyl)-2-propen-1-one |
| 1-050 | 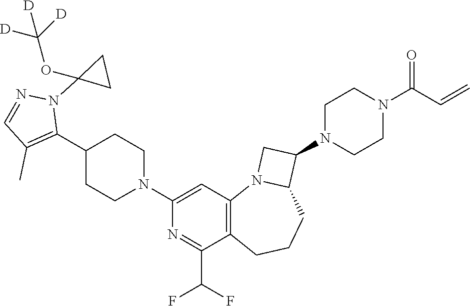 [SMILES: [2H]C([2H])([2H])OC1(n2ncc(C)c2C2CCN(c3cc4c(c(C(F)F)n3)CCC[C@H]3[C@H](N5CCN(C(=O)C=C)CC5)CN43)CC2)CC1] | 1-(4-((7aS,8R)-4-(difluoromethyl)-2-(4-(1-(1- (methoxy-d3)cyclopropyl)-4-methyl-1H- pyrazol-5-yl)piperidin-1-yl)-5,6,7,7a,8,9- hexahydroazeto[1,2-a]pyrido[3,4-f]azepin-8- yl)piperazin-1-yl)prop-2-en-1-one |
| 1-051 | 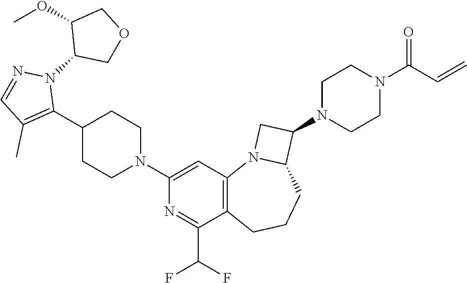 [SMILES: C=CC(=O)N1CCN([C@@H]2CN3c4cc(N5CCC(c6c(C)cnn6[C@H]6COC[C@H]6OC)CC5)nc(C(F)F)c4CCC[C@@H]23)CC1] | 1-(4-((7aS,8R)-4-(difluoromethyl)-2-(4-(1- ((3S,4S)-4-methoxytetrahydro-3-furanyl)-4- methyl-1H-pyrazol-5-yl)-1-piperidinyl)- 5,6,7,7a,8,9-hexahydroazeto[1,2-a]pyrido[3,4- f]azepin-8-yl)-1-piperazinyl)-2-propen-1-one |
| 1-052 | 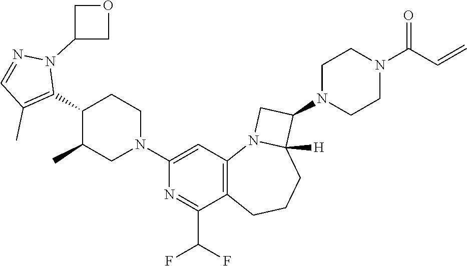 [SMILES: C=CC(=O)N1CCN([C@@H]2CN3c4cc(N5CC[C@@H](c6c(C)cnn6C6COC6)[C@H](C)C5)nc(C(F)F)c4CCC[C@@H]23)CC1] | 1-(4-((7aS,8R)-4-(difluoromethyl)-2-((35,4R)- 3-methyl-4-(4-methyl-1-(3-oxetanyl)-1H- pyrazol-5-yl)-1-piperidinyl)-5,6,7,7a,8,9- hexahydroazeto[1,2-a]pyrido[3,4-f]azepin-8-yl)- 1-piperazinyl)-2-propen-1-one |
| 1-053 | 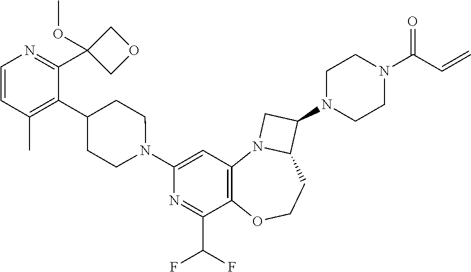 [SMILES: C=CC(=O)N1CCN([C@@H]2CN3c4cc(N5CCC(c6c(C)ccnc6C6(OC)COC6)CC5)nc(C(F)F)c4OCC[C@@H]23)CC1] | 1-(4-((7aS,8R)-4-(difluoromethyl)-2-(4-(2-(3- methoxy-3-oxetanyl)-4-methyl-3-pyridinyl)-1- piperidinyl)-7,7a,8,9-tetrahydro-6H-azeto[1,2- d]pyrido[3,4-b][1,4]oxazepin-8-yl)-1- piperazinyl)-2-propen-1-one |
| 1-054 | 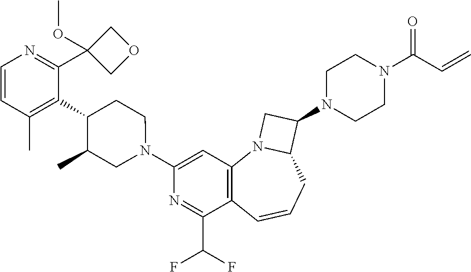 [SMILES: C=CC(=O)N1CCN([C@@H]2CN3c4cc(N5CC[C@@H](c6c(C)ccnc6C6(OC)COC6)[C@H](C)C5)nc(C(F)F)c4C=CC[C@@H]23)CC1] | 1-(4-((7aS,8R)-4-(difluoromethyl)-2-((3S,4R)- 4-(2-(3-methoxy-3-oxetanyl)-4-methyl-3- pyridinyl)-3-methyl-1-piperidinyl)-7,7a,8,9- tetrahydroazeto[1,2-a]pyrido[3,4-f]azepin-8-yl)- 1-piperazinyl)-2-propen-1-one |
| 1-055 | 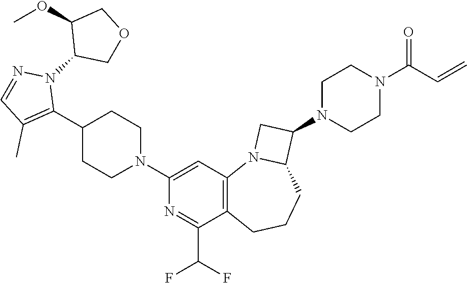 [SMILES: C=CC(=O)N1CCN([C@@H]2CN3c4cc(N5CCC(c6c(C)cnn6[C@H]6COC[C@@H]6OC)CC5)nc(C(F)F)c4CCC[C@@H]23)CC1] | 1-(4-((7aS,8R)-4-(difluoromethyl)-2-(4-(1- ((3S,4R)-4-methoxytetrahydro-3-furanyl)-4- methyl-1H-pyrazol-5-yl)-1-piperidinyl)- 5,6,7,7a,8,9-hexahydroazeto[1,2-a]pyrido[3,4- f]azepin-8-yl)-1-piperazinyl)-2-propen-1-one |
| 1-056 | 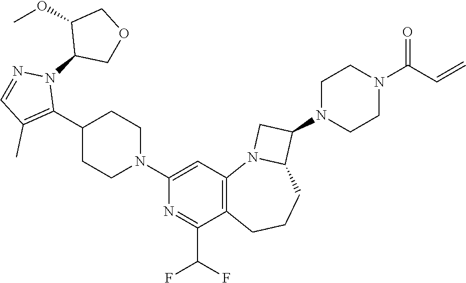 [SMILES: C=CC(=O)N1CCN([C@@H]2CN3c4cc(N5CCC(c6c(C)cnn6[C@@H]6COC[C@H]6OC)CC5)nc(C(F)F)c4CCC[C@@H]23)CC1] | 1-(4-((7aS,8R)-4-(difluoromethyl)-2-(4-(1- ((3R,4S)-4-methoxytetrahydro-3-furanyl)-4- methyl-1H-pyrazol-5-yl)-1-piperidinyl)- 5,6,7,7a,8,9-hexahydroazeto[1,2-a]pyrido[3,4- f]azepin-8-yl)-1-piperazinyl)-2-propen-1-one |
| 1-057 | 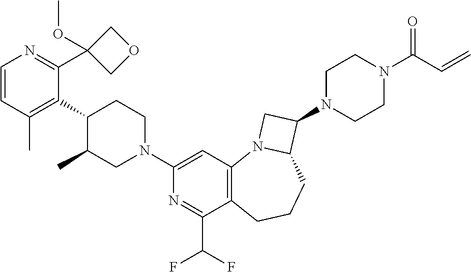 [SMILES: C=CC(=O)N1CCN([C@@H]2CN3c4cc(N5CC[C@@H](c6c(C)ccnc6C6(OC)COC6)[C@H](C)C5)nc(C(F)F)c4CCC[C@@H]23)CC1] | 1-(4-((7aS,8R)-4-(difluoromethyl)-2-((3S,4R)- 4-(2-(3-methoxy-3-oxetanyl)-4-methyl-3- pyridinyl)-3-methyl-1-piperidinyl)-5,6,7,7a,8,9- hexahydroazeto[1,2-a]pyrido[3,4-f]azepin-8-yl)- 1-piperazinyl)-2-propen-1-one |
| 1-058 | 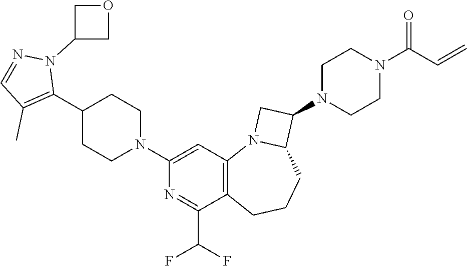 [SMILES: C=CC(=O)N1CCN([C@@H]2CN3c4cc(N5CCC(c6c(C)cnn6C6COC6)CC5)nc(C(F)F)c4CCC[C@@H]23)CC1] | 1-(4-((7aS,8R)-4-(difluoromethyl)-2-(4-(4- methyl-1-(3-oxetanyl)-1H-pyrazol-5-yl)-1- piperidinyl)-5,6,7,7a,8,9-hexahydroazeto[1,2- a]pyrido[3,4-f]azepin-8-yl)-1-piperazinyl)-2- propen-1-one |
| 1-059 | 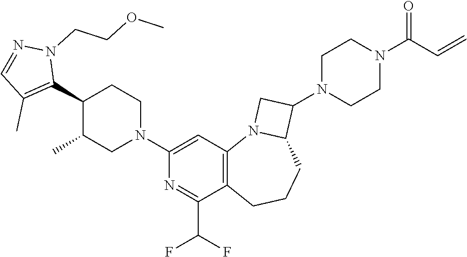 [SMILES: C=CC(=O)N1CCN(C2CN3c4cc(N5CC[C@H](c6c(C)cnn6CCOC)[C@@H](C)C5)nc(C(F)F)c4CCC[C@@H]23)CC1] | 1-(4-((7aS,8R)-4-(difluoromethyl)-2-((3R,4S)- 4-(1-(2-methoxyethyl)-4-methyl-1H-pyrazol-5- yl)-3-methyl-1-piperidinyl)-5,6,7,7a,8,9- hexahydroazeto[1,2-a]pyrido[3,4-f]azepin-8-yl)- 1-piperazinyl)-2-propen-1-one |
| 1-060 | 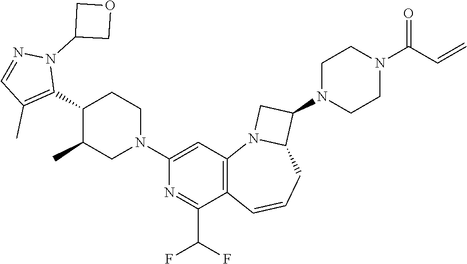 [SMILES: C=CC(=O)N1CCN([C@@H]2CN3c4cc(N5CC[C@@H](c6c(C)cnn6C6COC6)[C@H](C)C5)nc(C(F)F)c4C=CC[C@@H]23)CC1] | 1-(4-((7aS,8R)-4-(difluoromethyl)-2-((3S,4R)- 3-methyl-4-(4-methyl-1-(3-oxetanyl)-1H- pyrazol-5-yl)-1-piperidinyl)-7,7a,8,9- tetrahydroazeto[1,2-a]pyrido[3,4-f]azepin-8-yl)- 1-piperazinyl)-2-propen-1-one |
| 1-061 | 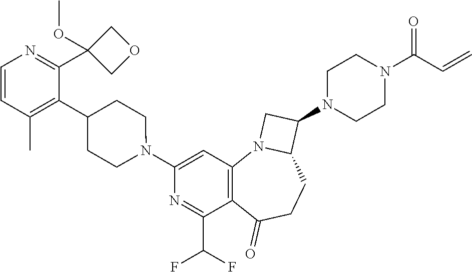 [SMILES: C=CC(=O)N1CCN([C@@H]2CN3c4cc(N5CCC(c6c(C)ccnc6C6(OC)COC6)CC5)nc(C(F)F)c4C(=O)CC[C@@H]23)CC1] | (7aS,8R)-4-(difluoromethyl)-2-(4-(2-(3- methoxy-3-oxetanyl)-4-methyl-3-pyridiny])-1- piperidinyl)-8-(4-(2-propenoyl)-1-piperazinyl)- 7,7a,8,9-tetrahydroazeto[1,2-a]pyrido[3,4- f]azepin-5(6H)-one |
| 1-062 | 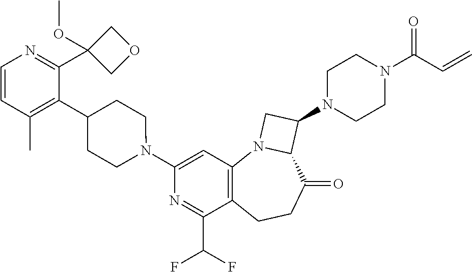 [SMILES: C=CC(=O)N1CCN([C@@H]2CN3c4cc(N5CCC(c6c(C)ccnc6C6(OC)COC6)CC5)nc(C(F)F)c4CCC(=O)[C@@H]23)CC1] | (7aR,SR)-4-(difluoromethyl)-2-(4-(2-(3- methoxy-3-oxetanyl)-4-methyl-3-pyridinyl)-1- piperidinyl)-8-(4-(2-propenoyl)-1-piperazinyl)- 5,6,8,9-tetrahydroazeto[1,2-a]pyrido[3,4- f]azepin-7(7aH)-one |
| 1-063 | 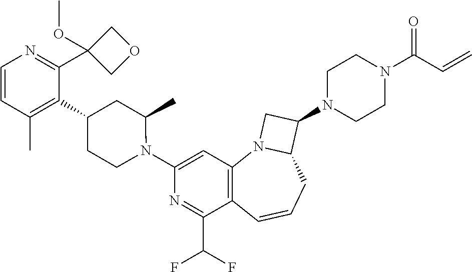 [SMILES: C=CC(=O)N1CCN([C@@H]2CN3c4cc(N5CC[C@H](c6c(C)ccnc6C6(OC)COC6)C[C@H]5C)nc(C(F)F)c4C=CC[C@@H]23)CC1] | 1-(4-((7aS,8R)-4-(difluoromethyl)-2-((2R,4S)- 4-(2-(3-methoxy-3-oxetanyl)-4-methyl-3- pyridinyl)-2-methyl-1-piperidinyl)-7,7a,8,9- tetrahydroazeto[1,2-a]pyrido[3,4-f]azepin-8-yl)- 1-piperazinyl)-2-propen-1-one |
| 1-064 | 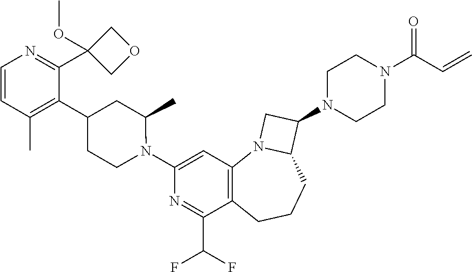 [SMILES: C=CC(=O)N1CCN([C@@H]2CN3c4cc(N5CCC(c6c(C)ccnc6C6(OC)COC6)C[C@H]5C)nc(C(F)F)c4CCC[C@@H]23)CC1] | 1-(4-((7aS,8R)-4-(difluoromethyl)-2-((2R,4S)- 4-(2-(3-methoxy-3-oxetanyl)-4-methyl-3- pyridinyl)-2-methyl-1-piperidinyl)-5,6,7,7a,8,9- hexahydroazeto[1,2-a]pyrido[3,4-f]azepin-8-yl)- 1-piperazinyl)-2-propon-1-one |
| 1-065 | 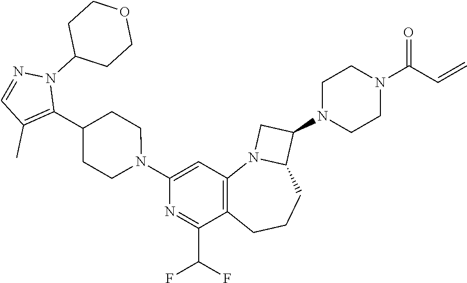 [SMILES: C=CC(=O)N1CCN([C@@H]2CN3c4cc(N5CCC(c6c(C)cnn6C6CCOCC6)CC5)nc(C(F)F)c4CCC[C@@H]23)CC1] | 1-(4-((7aS,8R)-4-(difluoromethyl)-2-(4-(4- methyl-1-(tetrahydro-2H-pyran-4-yl)-1H- pyrazol-5-yl)-1-piperidinyl)-5,6,7,7a,8,9- hexahydroazeto[1,2-a]pyrido[3,4-f]azepin-8-yl)- 1-piperazinyl)-2-propen-1-one |
| 1-066 | 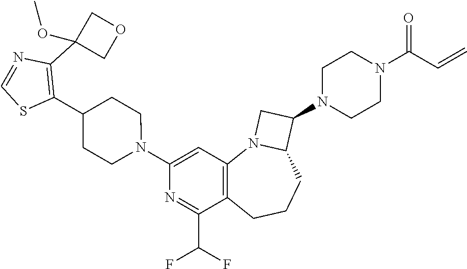 [SMILES: C=CC(=O)N1CCN([C@@H]2CN3c4cc(N5CCC(c6scnc6C6(OC)COC6)CC5)nc(C(F)F)c4CCC[C@@H]23)CC1] | 1-(4-((7aS,8R)-4-(difluoromethyl)-2-(4-(4-(3- methoxy-3-oxetanyl)-1,3-thiazol-5-yl)-1- piperidinyl)-5,6,7,7a,8,9-hexahydroazeto[1,2- a]pyrido[3,4-f]azepin-8-yl)-1-piperazinyl)-2- propen-1-one |
| 1-067 | 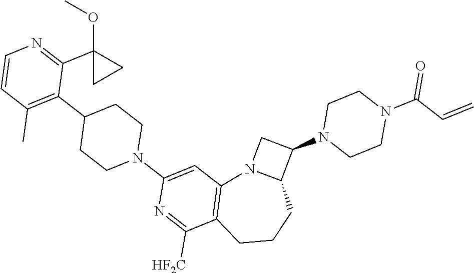 | 1-(4-((7aS,8R)-4-(difluoromethyl)-2-(4-(2-(1- methoxycyclopropyl)-4-methyl-3-pyridinyl)-1- piperidinyl)-5,6,7,7a,8,9-hexahydroazeto[1,2- a]pyrido[3,4-f]azepin-8-yl)-1-piperazinyl)-2- propen-1-one |
| 1-068 | 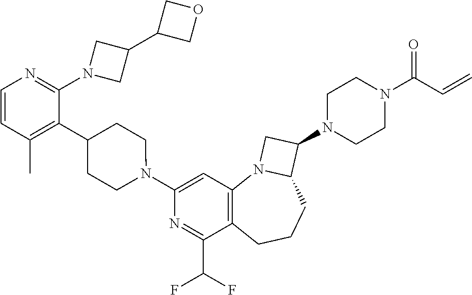 [SMILES: C=CC(=O)N1CCN([C@@H]2CN3c4cc(N5CCC(c6c(C)ccnc6N6CC(C7COC7)C6)CC5)nc(C(F)F)c4CCC[C@@H]23)CC1] | 1-(4-((7aS,8R)-4-(difluoromethyl)-2-(4-(4- methyl-2-(3-(3-oxetanyl)-1-azetidinyl)-3- pyridinyl)-1-piperidinyl)-5,6,7,7a,8,9- hexahydroazeto[1,2-a]pyrido[3,4-f]azepin-8-yl)- 1-piperazinyl)-2-propen-1-one |
| 1-069 | 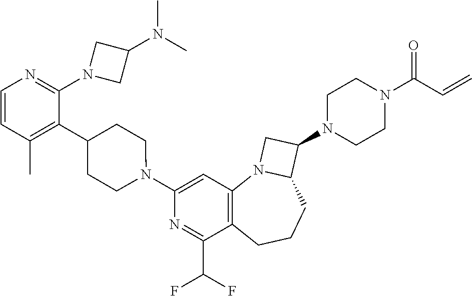 [SMILES: C=CC(=O)N1CCN([C@@H]2CN3c4cc(N5CCC(c6c(C)ccnc6N6CC(N(C)C)C6)CC5)nc(C(F)F)c4CCC[C@@H]23)CC1] | 1-(4-((7aS,8R)-4-(difluoromethyl)-2-(4-(2-(3- (dimethylamino)-1-azetidinyl)-4-methyl-3- pyridinyl)-1-piperidinyl)-5,6,7,7a,8,9- hexahydroazeto[1,2-a]pyrido[3,4-f]azepin-8-yl)- 1-piperazinyl)-2-propen-1-one |
| 1-070 | 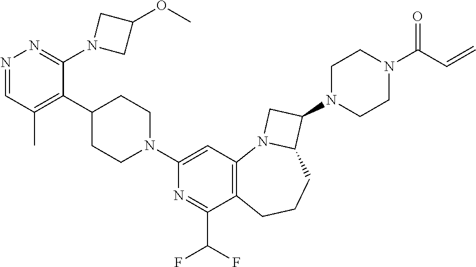 [SMILES: C=CC(=O)N1CCN([C@@H]2CN3c4cc(N5CCC(c6c(C)cnnc6N6CC(OC)C6)CC5)nc(C(F)F)c4CCC[C@@H]23)CC1] | 1-(4-((7aS,8R)-4-(difluoromethyl)-2-(4-(2-(3- methoxy-1-azetidinyl)-4-methyl-3-pyridinyl)-1- piperidinyl)-5,6,7,7a,8,9-hexahydroazeto[1,2- a]pyrido[3,4-f]azepin-8-yl)-1-piperazinyl)-2- propen-1-one |
| 1-072 | 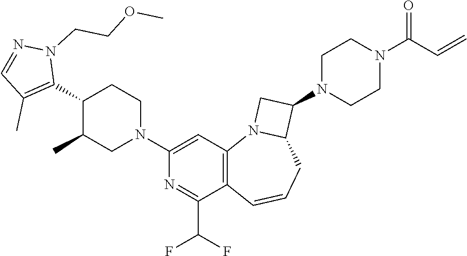 [SMILES: C=CC(=O)N1CCN([C@@H]2CN3c4cc(N5CC[C@@H](c6c(C)cnn6CCOC)[C@H](C)C5)nc(C(F)F)c4C=CC[C@@H]23)CC1] | 1-(4-((7aS,8R)-4-(difluoromethyl)-2-((3S,4R)- 4-(1-(2-methoxyethyl)-4-methyl-1H-pyrazol-5- yl)-3-methyl-1-piperidinyl)-7,7a,8,9- tetrahydroazeto[1,2-a]pyrido[3,4-f]azepin-8-yl)- 1-piperazinyl)-2-propen-1-one |
| 1-073 | 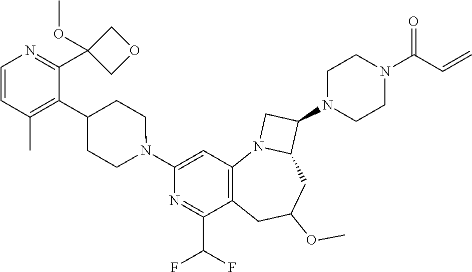 | 1-(4-(7aS,8R)-4-(difluoromethyl)-6-methoxy-2- (4-(2-(3-methoxyoxetan-3-yl)-4-methylpyridin- 3-yl)piperidin-1-yl)-5,6,7,7a,8,9- hexahydroazeto[1,2-a]pyrido[3,4-f]azepin-8- yl)piperazin-1-yl)prop-2-en-1-one |
| 1-074 | 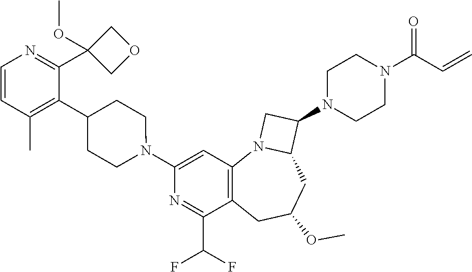 [SMILES: C=CC(=O)N1CCN([C@@H]2CN3c4cc(N5CCC(c6c(C)ccnc6C6(OC)COC6)CC5)nc(C(F)F)c4C[C@@H](OC)C[C@@H]23)CC1] | 1-(4-((6R,7aS,8R)-4-(difluoromethyl)-6- methoxy-2-(4-(2-(3-methoxy-3-oxetanyl)-4- methyl-3-pyridinyl)-1-piperidinyl)-5,6,7,7a,8,9- hexahydroazeto[1,2-a]pyrido[3,4-f]azepin-8-yl)- 1-piperazinyl)-2-propen-1-one |
| 1-075 | 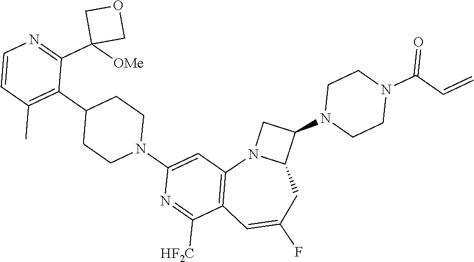 | 1-(4-((7aS,8R)-4-(difluoromethyl)-6,6-difluoro- 2-(4-(2-(3-methoxy-3-oxetanyl)-4-methyl-3- pyridinyl)-1-piperidinyl)-5,6,7,7a,8,9- hexahydroazeto[1,2-a]pyrido[3,4-f]azepin-8-yl)- 1-piperazinyl)-2-propen-1-one |
| 1-076 | 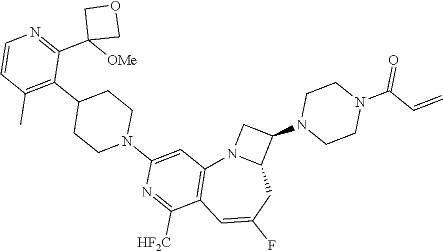 | 1-(4-((7aS,8R)-4-(difluoromethyl)-6-fluoro-2- (4-(2-(3-methoxy-3-oxetanyl)-4-methyl-3- pyridinyl)-1-piperidinyl)-7,7a,8,9- tetrahydroazeto[1,2-a]pyrido[3,4-f]azepin-8-yl)- 1-piperazinyl)-2-propen-1-one |
| 1-077 | 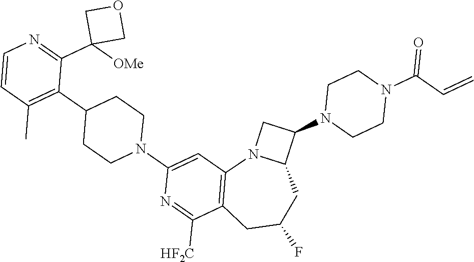 | 1-(4-((6R,7aS,8R)-4-(difluoromethyl)-6-fluoro- 2-(4-(2-(3-methoxy-3-oxetanyl)-4-methyl-3- pyridinyl)-1-piperidinyl)-5,6,7,7a,8,9- hexahydroazeto[1,2-a]pyrido[3,4-f]azepin-8-yl)- 1-piperazinyl)-2-propen-1-one |
| 1-078 | 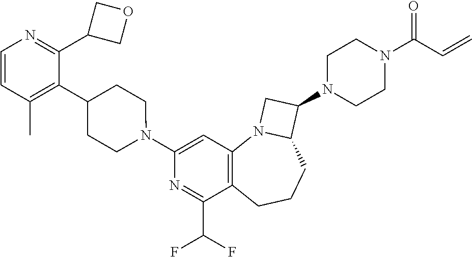 [SMILES: C=CC(=O)N1CCN([C@@H]2CN3c4cc(N5CCC(c6c(C)ccnc6C6COC6)CC5)nc(C(F)F)c4CCC[C@@H]23)CC1] | 1-(4-((7aS,8R)-4-(difluoromethyl)-2-(4-(4- methyl-2-(1-oxa-6-azaspiro[3.3]heptan-6-yl)-3- pyridinyl)-1-piperidinyl)-5,6,7,7a,8,9- hexahydroazeto[1,2-a]pyrido[3,4-f]azepin-8-yl)- 1-piperazinyl)-2-propen-1-one |
| 1-079 | 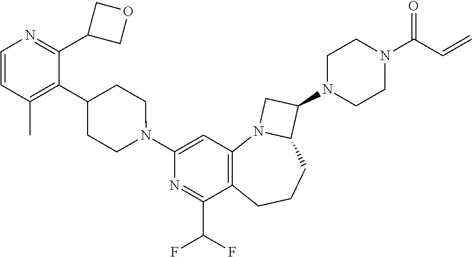 [SMILES: C=CC(=O)N1CCN([C@@H]2CN3c4cc(N5CCC(c6c(C)ccnc6C6COC6)CC5)nc(C(F)F)c4CCC[C@@H]23)CC1] | 1-(4-((7aS,8R)-4-(difluoromethyl)-2-(4-(4- methyl-2-(3-oxetanyl)-3-pyridinyl)-1- piperidinyl)-5,6,7,7a,8,9-hexahydroazeto[1,2- a]pyrido[3,4-f]azepin-8-yl)-1-piperazinyl)-2- propen-1-one |
| 1-080 | 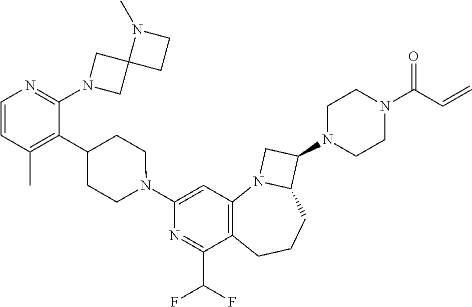 [SMILES: C=CC(=O)N1CCN([C@@H]2CN3c4cc(N5CCC(c6c(C)ccnc6N6CC7(CCN7C)C6)CC5)nc(C(F)F)c4CCC[C@@H]23)CC1] | 1-(4-((7aS,8R)-4-(difluoromethyl)-2-(4-(4- methyl-2-(1-methyl-1,6-diazaspiro[3.3]heptan- 6-yl)-3-pyridinyl)-1-piperidinyl)-5,6,7,7a,8,9- hexahydroazeto[1,2-a]pyrido[3,4-f]azepin-8-yl)- 1-piperazinyl)-2-propen-1-one |
| 1-081 | 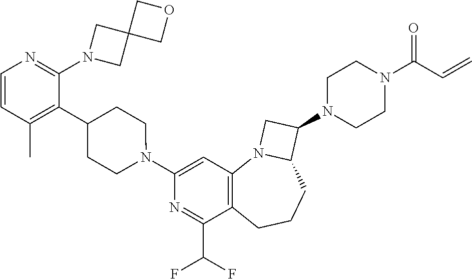 [SMILES: C=CC(=O)N1CCN([C@@H]2CN3c4cc(N5CCC(c6c(C)ccnc6N6CC7(COC7)C6)CC5)nc(C(F)F)c4CCC[C@@H]23)CC1] | 1-(4-((7aS,8R)-4-(difluoromethyl)-2-(4-(4- methyl-2-(2-oxa-6-azaspiro[3.3]heptan-6-yl)-3- pyridinyl)-1-piperidinyl)-5,6,7,7a,8,9- hexahydroazeto[1,2-a]pyrido[3,4-f]azepin-8-yl)- 1-piperazinyl)-2-propen-1-one |
| 1-082 | 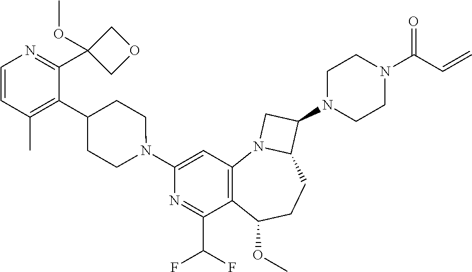 [SMILES: C=CC(=O)N1CCN([C@@H]2CN3c4cc(N5CCC(c6c(C)ccnc6C6(OC)COC6)CC5)nc(C(F)F)c4[C@@H](OC)CC[C@@H]23)CC1] | 1-(4-((5S,7aS,8R)-4-(difluoromethyl)-5- methoxy-2-(4-(2-(3-methoxy-3-oxetanyl)-4- methyl-3-pyridinyl)-1-piperidinyl)-5,6,7,7a,8,9- hexahydroazeto[1,2-a]pyrido[3,4-f]azepin-8-yl)- 1-piperazinyl)-2-propen-1-one |
| 1-083 | 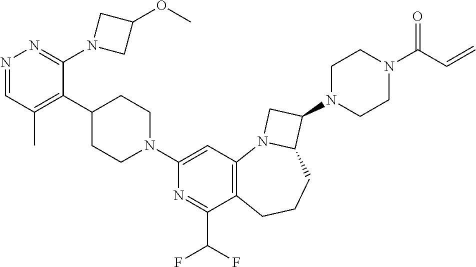 [SMILES: C=CC(=O)N1CCN([C@@H]2CN3c4cc(N5CCC(c6c(C)cnnc6N6CC(OC)C6)CC5)nc(C(F)F)c4CCC[C@@H]23)CC1] | 1-(4-((7aS,8R)-4-(difluoromethyl)-2-(4-(3-(3- methoxy-1-azetidinyl)-5-methyl-4-pyridazinyl)- 1-piperidinyl)-7,7a,8,9-tetrahydroazeto[1,2- a]pyrido[3,4-f]azepin-8-yl)-1-piperazinyl)-2- propen-1-one |
| 1-084 | 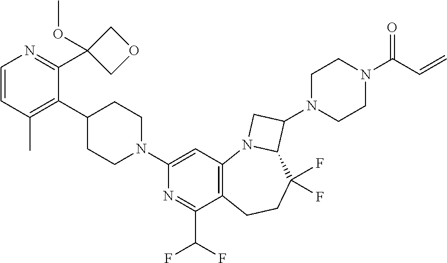 [SMILES: C=CC(=O)N1CCN(C2CN3c4cc(N5CCC(c6c(C)ccnc6C6(OC)COC6)CC5)nc(C(F)F)c4CCC(F)(F)[C@@H]23)CC1] | 1-(4-((7aR,8R)-4-(difluoromethyl)-7,7-difluoro- 2-(4-(2-(3-methoxy-3-oxetanyl)-4-methyl-3- pyridinyl)-1-piperidinyl)-5,6,7,7a,8,9- hexahydroazeto[1,2-a]pyrido[3,4-f]azepin-8-y])- 1-piperazinyl)-2-propen-1-one |
| 1-085 | 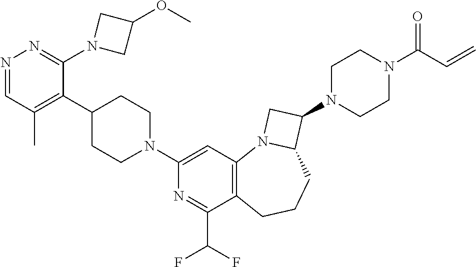 [SMILES: C=CC(=O)N1CCN([C@@H]2CN3c4cc(N5CCC(c6c(C)cnnc6N6CC(OC)C6)CC5)nc(C(F)F)c4CCC[C@@H]23)CC1] | 1-(4-((7aS,SR)-4-(difluoromethyl)-2-(4-(3-(3- methoxy-1-azetidinyl)-5-methyl-4-pyridazinyl)- 1-piperidinyl)-5,6,7,7a,8,9-hexahydroazeto[1,2- a]pyrido[3,4-f]azepin-8-yl)-1-piperazinyl)-2- propen-1-one |
| 1-086 | 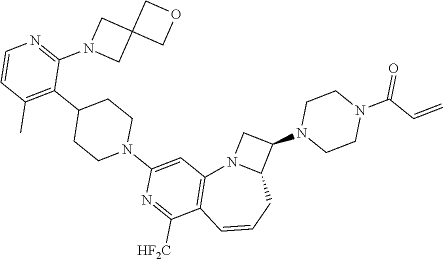 | 1-(4-((7aS,8R)-4-(Difluoromethyl)-2-(4-(5- methyl-3-(2-oxa-6-azaspiro[3.3]heptan-6-y])-4- pyridazinyl)-1-piperidinyl)-7,7a,8,9- tetrahydroazeto[1,2-a]pyrido[3,4-f]azepin-8-yl)- 1-piperazinyl)-2-propen-1-one |
| 1-087 | 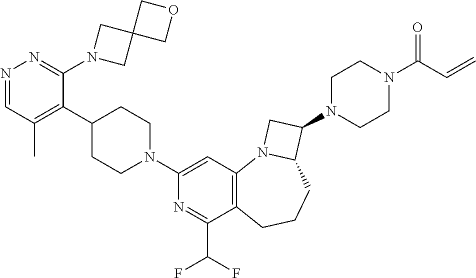 [SMILES: C=CC(=O)N1CCN([C@@H]2CN3c4cc(N5CCC(c6c(C)cnnc6N6CC7(COC7)C6)CC5)nc(C(F)F)c4CCC[C@@H]23)CC1] | 1-(4-((7aS,8R)-4-(difluoromethyl)-2-(4-(5- methyl-3-(2-oxa-6-azaspiro[3.3]heptan-6-yl)-4- pyridazinyl)-1-piperidinyl)-5,6,7,7a,8,9- hexahydroazeto[1,2-a]pyrido[3,4-f]azepin-8-yl)- 1-piperazinyl)-2-propen-1-one |
| 1-088 | 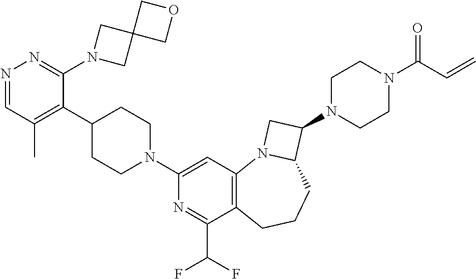 [SMILES: C=CC(=O)N1CCN([C@@H]2CN3c4cc(N5CCC(c6c(C)cnnc6N6CC7(COC7)C6)CC5)nc(C(F)F)c4CCC[C@@H]23)CC1] | 1-(4-((7aS,8R)-4-(difluoromethyl)-2-(4-(1-(1- methoxycyclopropyl)-4-methyl-1H-pyrazol-5- yl)-1-piperidinyl)-5,6,7,7a,8,9- hexahydroazeto[1,2-a]pyrido[3,4-f]azepin-8-yl)- 1-piperazinyl)-2-propen-1-one |
| 1-089 | 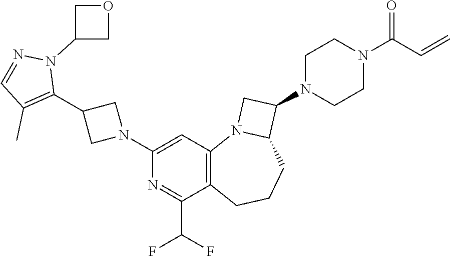 [SMILES: C=CC(=O)N1CCN([C@@H]2CN3c4cc(N5CC(c6c(C)cnn6C6COC6)C5)nc(C(F)F)c4CCC[C@@H]23)CC1] | 1-(4-((7aS,8R)-4-(difluoromethyl)-2-(3-(4- methyl-1-(3-oxetanyl)-1H-pyrazol-5-yl)-1- azetidinyl)-5,6,7,7a,8,9-hexahydroazeto[1,2- a]pyrido[3,4-f]azepin-8-yl)-1-piperazinyl)-2- propen-1-one |
| 1-090 | 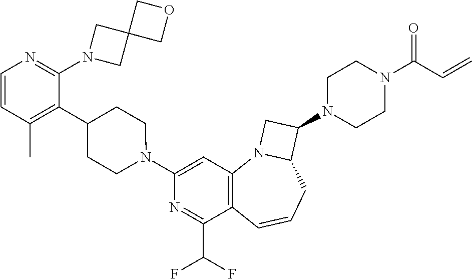 [SMILES: C=CC(=O)N1CCN([C@@H]2CN3c4cc(N5CCC(c6c(C)ccnc6N6CC7(COC7)C6)CC5)nc(C(F)F)c4C=CC[C@@H]23)CC1] | 1-(4-((7aS,8R)-4-(difluoromethyl)-2-(4-(4- methyl-2-(2-oxa-6-azaspiro[3.3]heptan-6-yl)-3- pyridinyl)-1-piperidinyl)-7,7a,8,9- tetrahydroazeto[1,2-a]pyrido[3,4-f]azepin-8-yl)- 1-piperazinyl)-2-propen-1-one |
| 1-091 | 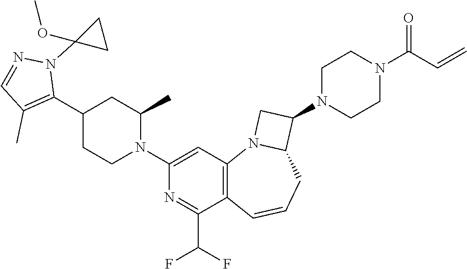 [SMILES: C=CC(=O)N1CCN([C@@H]2CN3c4cc(N5CCC(c6c(C)cnn6C6(OC)CC6)C[C@H]5C)nc(C(F)F)c4C=CC[C@@H]23)CC1] | 1-(4-((7aS,8R)-4-(difluoromethyl)-2-((2R,4S)- 4-(1-(1-methoxycyclopropyl)-4-methyl-1H- pyrazol-5-yl)-2-methyl-1-piperidinyl)-7,7a,8,9- tetrahydroazeto[1,2-a]pyrido[3,4-f]azepin-8-yl)- 1-piperazinyl)-2-propen-1-one |
| 1-092 | 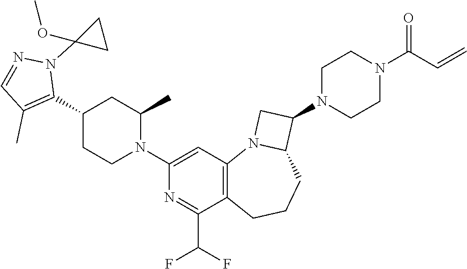 [SMILES: C=CC(=O)N1CCN([C@@H]2CN3c4cc(N5CC[C@H](c6c(C)cnn6C6(OC)CC6)C[C@H]5C)nc(C(F)F)c4CCC[C@@H]23)CC1] | 1-(4-((7aS,8R)-4-(difluoromethyl)-2-((2R,4S)- 4-(1-(1-methoxycyclopropyl)-4-methyl-1H- pyrazol-5-yl)-2-methyl-1-piperidinyl)- 5,6,7,7a,8,9-hoxahydroazeto[1,2-a]pyrido[3,4- f]azepin-8-yl)-1-piperazinyl)-2-propen-1-one |
| 1-093 | 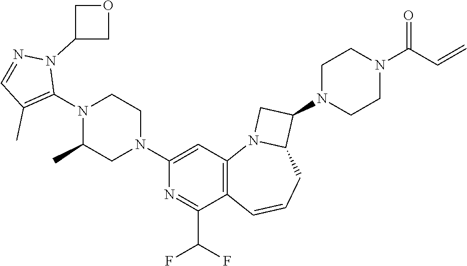 [SMILES: C=CC(=O)N1CCN([C@@H]2CN3c4cc(N5CCN(c6c(C)cnn6C6COC6)[C@H](C)C5)nc(C(F)F)c4C=CC[C@@H]23)CC1] | 1-(4-((7aS,8R)-4-(difluoromethyl)-2-((3R)-3- methyl-4-(4-methyl-1-(3-oxetany])-1H-pyrazol- 5-yl)-1-piperazinyl)-7,7a,8,9- tetrahydroazeto[1,2-a]pyrido[3,4-f]azepin-8-yl)- 1-piperazinyl)-2-propen-1-one |
| 1-094 | 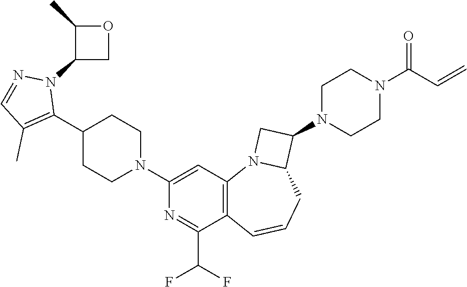 [SMILES: C=CC(=O)N1CCN([C@@H]2CN3c4cc(N5CCC(c6c(C)cnn6[C@@H]6CO[C@@H]6C)CC5)nc(C(F)F)c4C=CC[C@@H]23)CC1] | 1-(4-((7aS,8R)-4-(difluoromethyl)-2-(4-(4- methyl-1-((2R,3R)-2-methyl-3-oxetanyl)-1H- pyrazol-5-yl)-1-piperidinyl)-7,7a,8,9- tetrahydroazeto[1,2-a]pyrido[3,4-f]azepin-8-yl)- 1-piperazinyl)-2-propen-1-one |
| 1-095 | 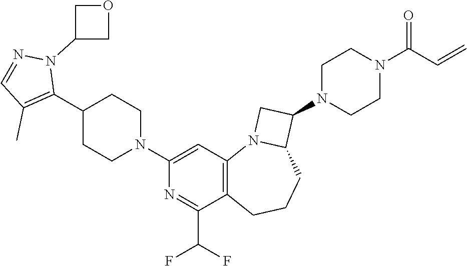 [SMILES: C=CC(=O)N1CCN([C@@H]2CN3c4cc(N5CCC(c6c(C)cnn6C6COC6)CC5)nc(C(F)F)c4CCC[C@@H]23)CC1] | 1-(4-((7aS,8R)-4-(difluoromethyl)-2-(4-(4- methyl-1-(3-oxetanyl)-1H-pyrazol-5-yl)-1- piperidinyl)-7,7a,8,9-tetrahydroazeto[1,2- a]pyrido[3,4-f]azepin-8-yl)-1-piperazinyl)-2- propen-1-one |
| 1-096 | 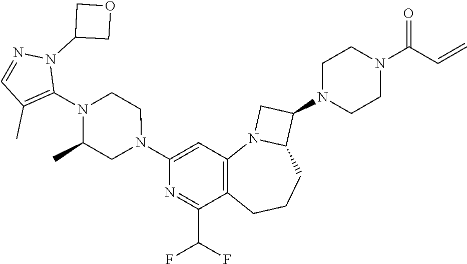 [SMILES: C=CC(=O)N1CCN([C@@H]2CN3c4cc(N5CCN(c6c(C)cnn6C6COC6)[C@H](C)C5)nc(C(F)F)c4CCC[C@@H]23)CC1] | 1-(4-((7aS,8R)-4-(difluoromethyl)-2-((3R)-3- methyl-4-(4-methyl-1-(3-oxetanyl)-1H-pyrazol- 5-yl)-1-piperazinyl)-5,6,7,7a,8,9- hexahydroazeto[1,2-a]pyrido[3,4-f]azepin-8-y])- 1-piperazinyl)-2-propen-1-one |
| 1-097 | 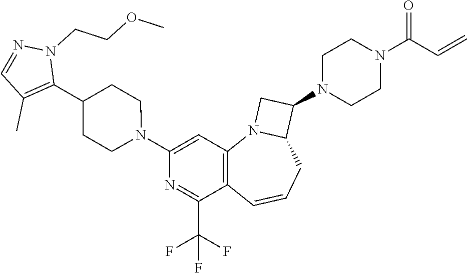 [SMILES: C=CC(=O)N1CCN([C@@H]2CN3c4cc(N5CCC(c6c(C)cnn6CCOC)CC5)nc(C(F)(F)F)c4C=CC[C@@H]23)CC1] | 1-(4-((7aS,8R)-2-(4-(1-(2-Methoxyethyl)-4- methyl-1H-pyrazol-5-yl)-1-piperidinyl)-4- (trifluoromethyl)-7,7a,8,9-tetrahydroazeto[1,2- a]pyrido[3,4-f]azepin-8-yl)-1-piperazinyl)-2- propen-1-one |
| 1-098 | 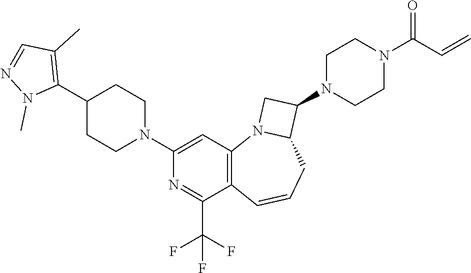 [SMILES: C=CC(=O)N1CCN([C@@H]2CN3c4cc(N5CCC(c6c(C)cnn6C)CC5)nc(C(F)(F)F)c4C=CC[C@@H]23)CC1] | 1-(4-((7aS,8R)-2-(4-(1,4-dimethyl-1H-pyrazol- 5-yl)-1-piperidinyl)-4-(trifluoromethyl)- 7,7a,8,9-tetrahydroazeto[1,2-a]pyrido[3,4- f]azepin-8-yl)-1-piperazinyl)-2-propen-1-one |
| 1-099 | 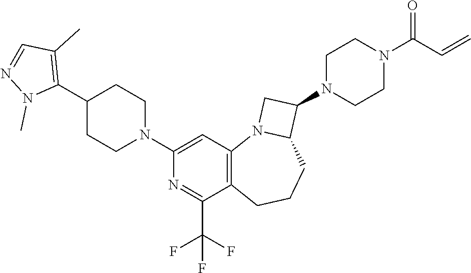 [SMILES: C=CC(=O)N1CCN([C@@H]2CN3c4cc(N5CCC(c6c(C)cnn6C)CC5)nc(C(F)(F)F)c4CCC[C@@H]23)CC1] | 1-(4-((7aS,8R)-2-(4-(1,4-dimethyl-1H-pyrazol- 5-yl)-1-piperidinyl)-4-(trifluoromethyl)- 5,6,7,7a,8,9-hexahydroazeto[1,2-a]pyrido[3,4- f]azepin-8-yl)-1-piperazinyl)-2-propen-1-one |
| 1-100 | 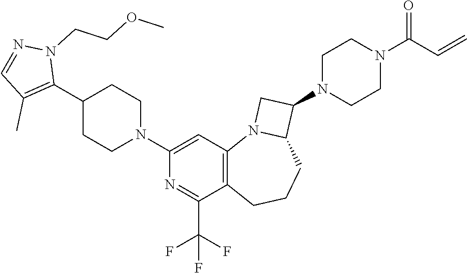 [SMILES: C=CC(=O)N1CCN([C@@H]2CN3c4cc(N5CCC(c6c(C)cnn6CCOC)CC5)nc(C(F)(F)F)c4CCC[C@@H]23)CC1] | 1-(4-((7aS,8R)-2-(4-(1-(2-methoxyethyl)-4- methyl-1H-pyrazol-5-yl)-1-piperidinyl)-4- (trifluoromethyl)-5,6,7,7a,8,9- hexahydroazeto[1,2-a]pyrido[3,4-f]azepin-8-y])- 1-piperazinyl)-2-propen-1-one |
| 1-101 | 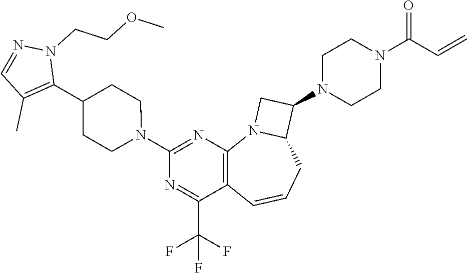 [SMILES: C=CC(=O)N1CCN([C@@H]2CN3c4nc(N5CCC(c6c(C)cnn6CCOC)CC5)nc(C(F)(F)F)c4C=CC[C@@H]23)CC1] | 1-(4-((7aS,8R)-2-(4-(1-(2-methoxyethyl)-4- methyl-1H-pyrazol-5-yl)-1-piperidinyl)-4- (trifluoromethyl)-7,7a,8,9-tetrahydroazeto[1,2- a]pyrimido[5,4-f]azepin-8-yl)-1-piperazinyl)-2- propen-1-one |
| 1-102 | 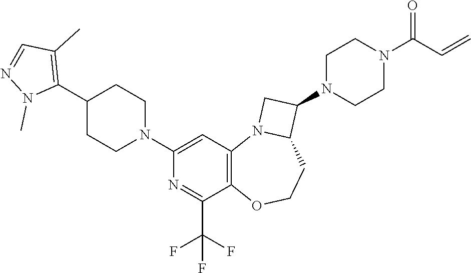 [SMILES: C=CC(=O)N1CCN([C@@H]2CN3c4cc(N5CCC(c6c(C)cnn6C)CC5)nc(C(F)(F)F)c4OCC[C@@H]23)CC1] | 1-(4-((7aS,8R)-2-(4-(1,4-dimethyl-1H-pyrazol- 5-yl)-1-piperidinyl)-4-(trifluoromethyl)- 7,7a,8,9-tetrahydro-6H-azeto[1,2-d]pyrido[3,4- b][1,4]oxazepin-8-y])-1-piperazinyl)-2-propen- 1-one |
| 1-103 | 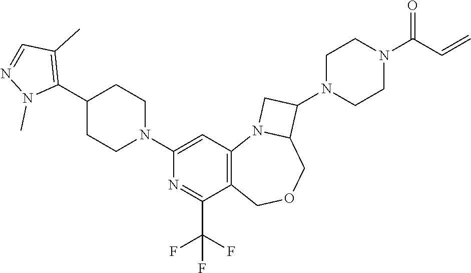 [SMILES: C=CC(=O)N1CCN(C2CN3c4cc(N5CCC(c6c(C)cnn6C)CC5)nc(C(F)(F)F)c4COCC23)CC1] | 1-(4-(2-(4-(1,4-dimethyl-1H-pyrazol-5- yl)piperidin-1-yl)-4-(trifluoromethyl)-7,7a,8,9- tetrahydro-5H-azeto[2,1-c]pyrido[4,3- e][1,4]oxazepin-8-yl)piperazin-1-yl)prop-2-en- 1-one |
| 1-104 | 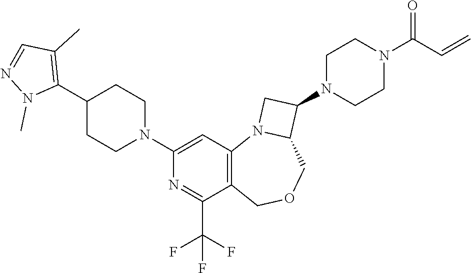 [SMILES: C=CC(=O)N1CCN([C@@H]2CN3c4cc(N5CCC(c6c(C)cnn6C)CC5)nc(C(F)(F)F)c4COC[C@@H]23)CC1] | 1-(4-((7aR,8R)-2-(4-(1,4-dimethyl-1H-pyrazol- 5-yl)-1-piperidinyl)-4-(trifluoromethyl)- 7,7a,8,9-tetrahydro-5H-azeto[2,1-c]pyrido[4,3- e][1,4]oxazepin-8-yl)-1-piperazinyl)-2-propen- 1-one |
| 1-105 | 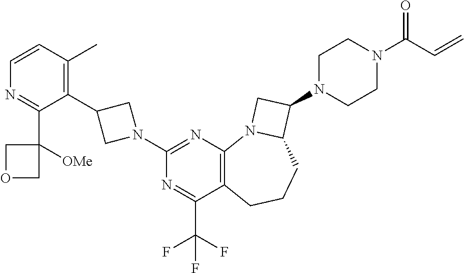 [SMILES: C=CC(=O)N1CCN([C@@H]2CN3c4nc(N5CC(c6c(C)ccnc6C6(OC)COC6)C5)nc(C(F)(F)F)c4CCC[C@@H]23)CC1] | 1-(4-((7aS,8R)-2-(3-(2-(3-methoxyoxetan-3-yl)- 4-methylpyridin-3-yl)azetidin-1-yl)-4- (trifluoromethyl)-5,6,7,7a,8,9- hexahydroazeto[1,2-a]pyrimido[5,4-f]azepin-8- yl)piperazin-1-yl)prop-2-en-1-one |
| 1-106 | 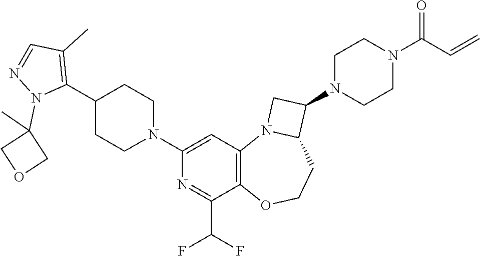 [SMILES: C=CC(=O)N1CCN([C@@H]2CN3c4cc(N5CCC(c6c(C)cnn6C6(C)COC6)CC5)nc(C(F)F)c4OCC[C@@H]23)CC1] | 1-(4-((7aS,8R)-4-(difluoromethyl)-2-(4-(4- methyl-1-(3-methyloxetan-3-yl)-1H-pyrazol-5- yl)piperidin-1-yl)-7,7a,8,9-tetrahydro-6H- azeto[1,2-d]pyrido[3,4-b][1,4]oxazepin-8- yl)piperazin-1-yl)prop-2-en-1-one |
| 1-107 | 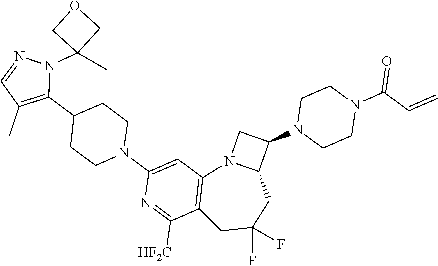 | 1-(4-((7aS,8R)-4-(difluoromethyl)-6,6-difluoro- 2-(4-(4-methyl-1-(3-methyloxetan-3-y])-1H- pyrazol-5-yl)piperidin-1-yl)-5,6,7,7a,8,9- hexahydroazeto[1,2-a]pyrido[3,4-f]azepin-8- yl)piperazin-1-yl)prop-2-en-1-one |
| 1-108 | 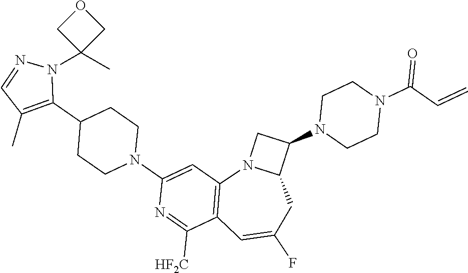 | 1-(4-((7aS,8R)-4-(difluoromethyl)-6-fluoro-2- (4-(4-methyl-1-(3-methyloxetan-3-yl)-1H- pyrazol-5-yl)piperidin-1-yl)-7,7a,8,9- tetrahydroazeto[1,2-a]pyrido[3,4-f]azepin-8- yl)piperazin-1-yl)prop-2-en-1-one |
| 1-109 | 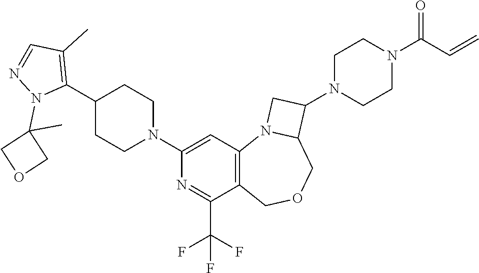 [SMILES: C=CC(=O)N1CCN(C2CN3c4cc(N5CCC(c6c(C)cnn6C6(C)COC6)CC5)nc(C(F)(F)F)c4COCC23)CC1] | 1 1-(4-(2-(4-(4-methyl-1-(3-methyloxetan-3-yl)- 1H-pyrazol-5-yl)piperidin-1-yl)-4- (trifluoromethyl)-7,7a,8,9-tetrahydro-5H- azeto[2,1-c]pyrido[4,3-c][1,4]oxazepin-8- yl)piperazin-1-yl)prop-2-en-1-one |

In some cases, the compound of Formula (II) is compound 1-001 through compound 1-109, or a pharmaceutically acceptable salt thereof, as shown in Table A′.

In some cases, X is

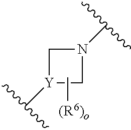

and contemplated compounds of Formula (II) include, for example, compounds 1-089 and 1-105, and pharmaceutically acceptable salts thereof.

In some cases, X, is

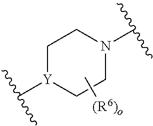

and contemplated compounds of Formula (II) include, for example, compounds 1-001 to 1-034, 1-036 to 1-070, 1-072 to 1-088, 1-090 to 1-104, and 1-106 to 1-109, and pharmaceutically acceptable salts thereof.

In some cases, Y is CH and Z is substituted pyrazolyl, and contemplated compounds of Formula (II) include, for example, compounds 1-002, 1-003, 1-006 to 1-010, 1-013 to 1-016, 1-018 to 1-021, 1-024 to 1-026, 1-028 to 1-030, 1-032 to 1-034, 1-037 to 1-041, 1-044 to 1-047, 1-049 to 1-052, 1-055, 1-056, 1-058 to 1-060, 1-065, 1-072, 1-088, 1-091, 1-092, 1-094, 1-095, 1-097 to 1-104, and 1-106 to 1-109, and pharmaceutically acceptable salts thereof.

In some cases, Y is CH and Z is substituted thiazolyl, and contemplated compounds of Formula (II) include, for example, compound 1-066, and pharmaceutically acceptable salts thereof.

In some cases, Y is CH and Z is substituted pyridyl, and contemplated compounds of Formula (II) include, for example, 1-001, 1-004, 1-005, 1-011, 1-012, 1-017, 1-027, 1-031, 1-036, 1-042, 1-043, 1-048, 1-053, 1-054, 1-057, 1-061 to 1-064, 1-067 to 1-070, 1-073 to 1-082, 1-084, 1-086, 1-090, and pharmaceutically acceptable salts thereof.

In some cases, Y is CH and Z is substituted pyridazinyl and contemplated compounds of Formula (II) include, for example, compounds 1-022, 1-023, 1-083, 1-085, and 1-087, and pharmaceutically acceptable salts thereof.

In some cases, Y is N, and contemplated compounds of Formula (II) include, for example, compounds 1-093 and 1-096, and pharmaceutically acceptable salts thereof.

In some cases, X is

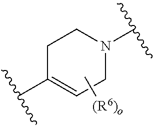

and contemplated compounds of Formula (II) include, for example, compound 1-035, and pharmaceutically acceptable salts thereof.

In some cases, the compound of Formula (II) is a compound listed in Table B′, below. If the stereochemistry of a structure or a portion of a structure in Table B′ is not explicitly shown (e.g., such as with dashed or bold lines), then the structure or portion of structure is either achiral or interpreted as being any of the possible stereoisomers of the structure or portion of the structure. In cases in which the stereochemistry of the structure or portion of the structure in Table B′ is explicitly shown, a single stereoisomer of the structure or portion of a structure is represented.

TABLE B′

| Ex. # | Chemical Structure | Name |
|---|---|---|
| 1-001 | 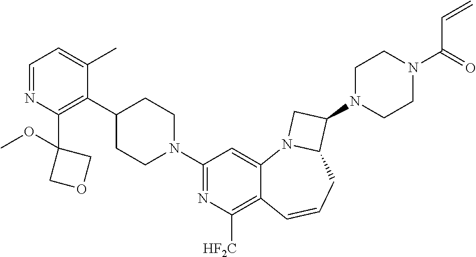 | 1-(4-((7aS,8R)-4-(difluoromethyl)-2-(4-(2-(3- methoxy-3-oxetanyl)-4-methyl-3-pyridinyl)-1- piperidinyl)-7,7a,8,9-tetrahydroazeto[1,2- a]pyrido[3,4-f]azepin-8-yl)-1-piperazinyl)-2- propen-1-one |
| 1-002 | 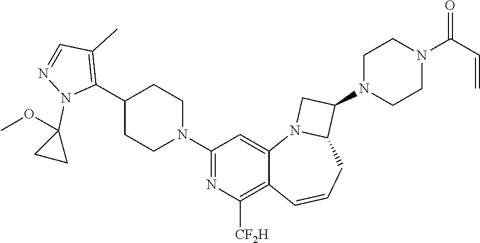 [SMILES: C=CC(=O)N1CCN([C@@H]2CN3c4cc(N5CCC(c6c(C)cnn6C6(OC)CC6)CC5)nc(C(F)F)c4C=CC[C@@H]23)CC1] | 1-(4-((7aS,8R)-4-(difluoromethyl)-2-(4-(1-(1- methoxycyclopropyl)-4-methyl-1H-pyrazol-5- yl)-1-piperidinyl)-7,7a,8,9-tetrahydroazeto[1,2- a]pyrido[3,4-f]azepin-8-yl)-1-piperazinyl)-2- propen-1-one |
| 1-003 | 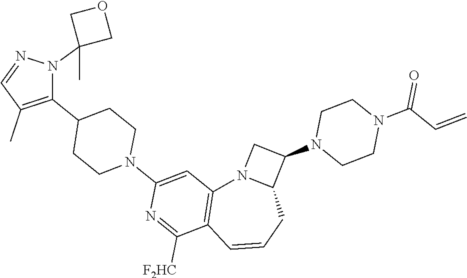 [SMILES: C=CC(=O)N1CCN([C@@H]2CN3c4cc(N5CCC(c6c(C)cnn6C6(C)COC6)CC5)nc(C(F)F)c4C=CC[C@@H]23)CC1] | 1-(4-((7aS,8R)-4-(difluoromethyl)-2-(4-(4- methyl-1-(3-methyl-3-oxetanyl)-1H-pyrazol-5- yl)-1-piperidinyl)-7,7a,8,9-tetrahydroazeto[1,2- a]pyrido[3,4-f]azepin-8-yl)-1-piperazinyl)-2- propen-1-one |
| 1-009 | 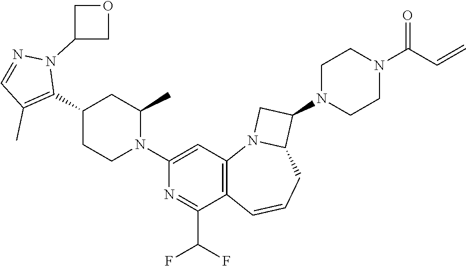 [SMILES: C=CC(=O)N1CCN([C@@H]2CN3c4cc(N5CC[C@H](c6c(C)cnn6C6COC6)C[C@H]5C)nc(C(F)F)c4C=CC[C@@H]23)CC1] | 1-(4-((7aS,8R)-4-(difluoromethyl)-2-((2R,4S)- 2-methyl-4-(4-methyl-1-(3-oxetanyl)-1H- pyrazol-5-yl)-1-piperidinyl)-7,7a,8,9- tetrahydroazeto[1,2-a]pyrido[3,4-f]azepin-8-yl)- 1-piperazinyl)-2-propen-1-one |
| 1-017 | 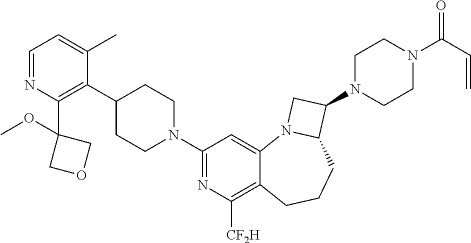 [SMILES: C=CC(=O)N1CCN([C@@H]2CN3c4cc(N5CCC(c6c(C)ccnc6C6(OC)COC6)CC5)nc(C(F)F)c4CCC[C@@H]23)CC1] | 1-(4-((7aS,8R)-4-(difluoromethyl)-2-(4-(2-(3- methoxy-3-oxetanyl)-4-methyl-3-pyridinyl)-1- piperidinyl)-5,6,7,7a,8,9-hexahydroazeto[1,2- a]pyrido[3,4-f]azepin-8-yl)-1-piperazinyl)-2- propen-1-one |
| 1-018 | 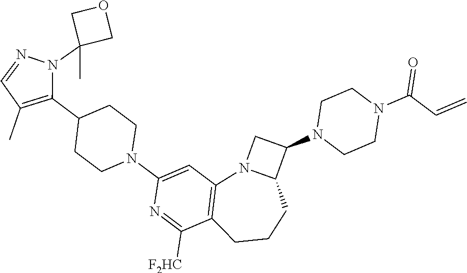 [SMILES: C=CC(=O)N1CCN([C@@H]2CN3c4cc(N5CCC(c6c(C)cnn6C6(C)COC6)CC5)nc(C(F)F)c4CCC[C@@H]23)CC1] | 1-(4-((7aS,8R)-4-(difluoromethyl)-2-(4-(4- methyl-1-(3-methyloxetan-3-yl)-1H-pyrazol-5- yl)piperidin-1-yl)-5,6,7,7a,8,9- hexahydroazeto[1,2-a]pyrido[3,4-f]azepin-8- yl)piperazin-1-yl)prop-2-en-1-one |
| 1-019 | 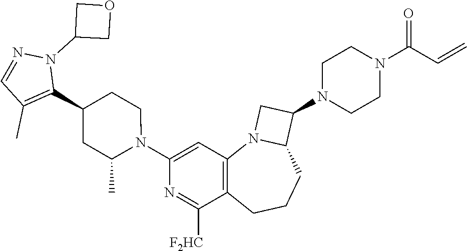 [SMILES: C=CC(=O)N1CCN([C@@H]2CN3c4cc(N5CC[C@H](c6c(C)cnn6C6COC6)C[C@H]5C)nc(C(F)F)c4CCC[C@@H]23)CC1] | 1-(4-((7aS,8R)-4-(difluoromethyl)-2-((2R,4S)- 2-methyl-4-(4-methyl-1-(3-oxetanyl)-1H- pyrazol-5-yl)-1-piperidinyl)-5,6,7,7a,8,9- hexahydroazeto[1,2-a]pyrido[3,4-f]azepin-8-yl)- 1-piperazinyl)-2-propen-1-one |
| 1-020 | 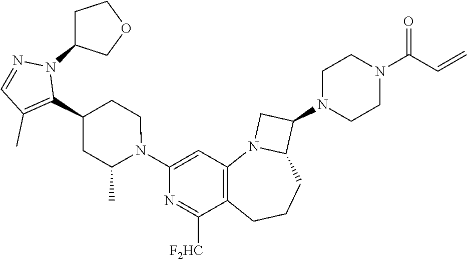 [SMILES: C=CC(=O)N1CCN([C@@H]2CN3c4cc(N5CC[C@H](c6c(C)cnn6[C@H]6CCOC6)C[C@H]5C)nc(C(F)F)c4CCC[C@@H]23)CC1] | 1-(4-((7aS,8R)-4-(difluoromethyl)-2-((2R,4S)- 2-methyl-4-(4-methyl-1-((3S)-tetrahydro-3- furanyl)-1H-pyrazol-5-yl)-1-piperidinyl)- 5,6,7,7a,8,9-hexahydroazeto[1,2-a]pyrido[3,4-f]azepin- 8-yl)-1-piperazinyl)-2-propen-1-one |
| 1-021 | 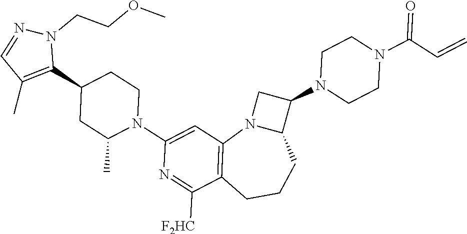 [SMILES: C=CC(=O)N1CCN([C@@H]2CN3c4cc(N5CC[C@H](c6c(C)cnn6CCOC)C[C@H]5C)nc(C(F)F)c4CCC[C@@H]23)CC1] | 1-(4-((7aS,8R)-4-(difluoromethyl)-2-((2R,4S)- 4-(1-(2-methoxyethyl)-4-methyl-1H-pyrazol-5- yl)-2-methyl-1-piperidinyl)-5,6,7,7a,8,9- hexahydroazeto[1,2-a]pyrido[3,4-f]azepin-8-yl)- 1-piperazinyl)-2-propen-1-one |
| 1-067 | 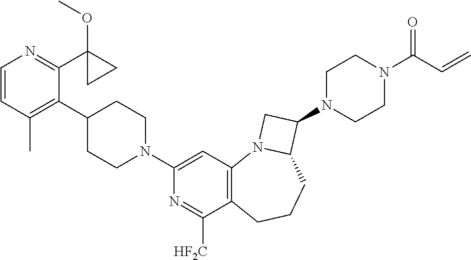 | 1-(4-((7aS,8R)-4-(difluoromethyl)-2-(4-(2-(1- methoxycyclopropyl)-4-methyl-3-pyridinyl)-1- piperidinyl)-5,6,7,7a,8,9- hexahydroazeto[1,2-a]pyrido[3,4-f]azepin-8-yl)-1- piperazinyl)-2-propen-1-one |
| 1-075 | 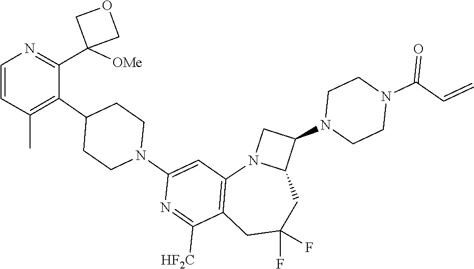 | 1-(4-((7aS,8R)-4-(difluoromethyl)-6,6-difluoro- 2-(4-(2-(3-methoxy-3-oxetanyl)-4-methyl-3- pyridinyl)-1-piperidinyl)-5,6,7,7a,8,9- hexahydroazeto[1,2-a]pyrido[3,4-f]azepin-8-yl)- 1-piperazinyl)-2-propen-1-one |
| 1-076 | 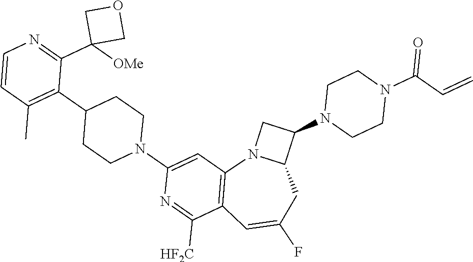 | 1-(4-((7aS,8R)-4-(difluoromethyl)-6-fluoro-2- (4-(2-(3-methoxy-3-oxetanyl)-4-methyl-3- pyridinyl)-1-piperidinyl)-7,7a,8,9- tetrahydroazeto[1,2-a]pyrido[3,4-f]azepin-8-yl)- 1-piperazinyl)-2-propen-1-one |
| 1-077 | 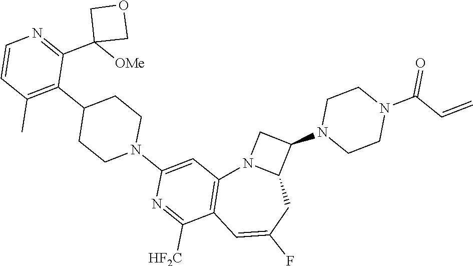 | 1-(4-((6R,7aS,8R)-4-(difluoromethyl)-6-fluoro- 2-(4-(2-(3-methoxy-3-oxetanyl)-4-methyl-3- pyridinyl)-1-piperidinyl)-5,6,7,7a,8,9- hexahydroazeto[1,2-a]pyrido[3,4-f]azepin-8-yl)- 1-piperazinyl)-2-propen-1-one |
| 1-107 | 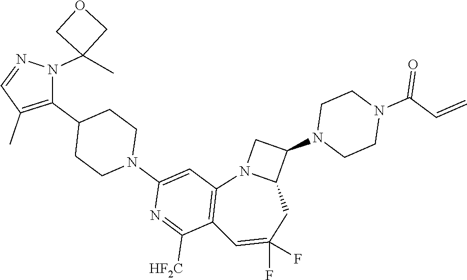 | 1-(4-((7aS,8R)-4-(difluoromethyl)-6,6-difluoro- 2-(4-(4-methyl-1-(3-methyloxetan-3-yl)-1H- pyrazol-5-yl)piperidin-1-yl)-5,6,7,7a,8,9- hexahydroazeto[1,2-a]pyrido[3,4-f]azepin-8- yl)piperazin-1-yl)prop-2-en-1-one |
| 1-108 | 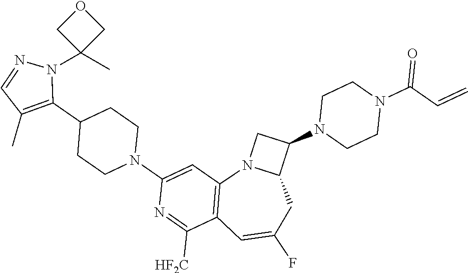 | 1-(4-((7aS,8R)-4-(difluoromethyl)-6-fluoro-2- (4-(4-methyl-1-(3-methyloxetan-3-yl)-1H- pyrazol-5-yl)piperidin-1-yl)-7,7a,8,9- tetrahydroazeto[1,2-a]pyrido[3,4-f]azepin-8- yl)piperazin-1-yl)prop-2-en-1-one |

In some cases, the compound of Formula (II) is compound 1-001, or a pharmaceutically acceptable salt thereof. In some cases, the compound of Formula (II) is compound 1-002, or a pharmaceutically acceptable salt thereof. In some cases, the compound of Formula (II) is compound 1-003, or a pharmaceutically acceptable salt thereof. In some cases, the compound of Formula (II) is compound 1-009, or a pharmaceutically acceptable salt thereof. In some cases, the compound of Formula (II) is compound 1-017, or a pharmaceutically acceptable salt thereof. In some cases, the compound of Formula (II) is compound 1-018, or a pharmaceutically acceptable salt thereof. In some cases, the compound of Formula (II) is compound 1-019, or a pharmaceutically acceptable salt thereof. In some cases, the compound of Formula (II) is compound 1-020, or a pharmaceutically acceptable salt thereof. In some cases, the compound of Formula (II) is compound 1-021, or a pharmaceutically acceptable salt thereof. In some cases, the compound of Formula (II) is compound 1-067, or a pharmaceutically acceptable salt thereof. In some cases, the compound of Formula (II) is compound 1-075, or a pharmaceutically acceptable salt thereof. In some cases, the compound of Formula (II) is compound 1-076, or a pharmaceutically acceptable salt thereof. In some cases, the compound of Formula (II) is compound 1-077, or a pharmaceutically acceptable salt thereof. In some cases, the compound of Formula (II) is compound 1-107, or a pharmaceutically acceptable salt thereof. In some cases, the compound of Formula (II) is compound 1-108, or a pharmaceutically acceptable salt thereof.

### Compounds of Formula (I)

In other embodiments, provided herein are compounds of Formula (I):

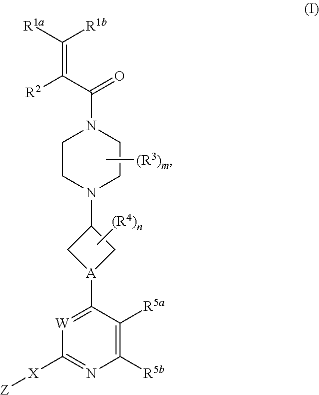

or a pharmaceutically acceptable salt thereof,
wherein:
m is 0, 1, 2, 3, or 4;
n is 1 or 2;
o is 0, 1, 2, 3, or 4;
A is N, CH, C-halo, C—CN, C—C₁₋₃alkyl, C—C₁₋₃haloalkyl, C—C₀₋₂alkyleneOH, or C—C₀₋₃alkylene-C₁₋₄alkoxy:
W is CH, C-halo, C—CN, C—C₁₋₃alkyl, C—C₁₋₃haloalkyl, C—C₀₋₂alkyleneOH, or C—C₀₋₃alkylene-C₁₋₄alkoxy;
X is

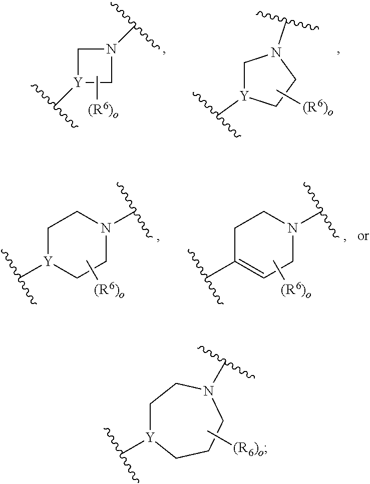

Y is N, C—H, C-halo, C—CN, C—C₁₋₃alkyl, C—C₁₋₃haloalkyl, C—C₀₋₂alkyleneOH, or C—C₀₋₃alkylene-C₁₋₄alkoxy;
Z is phenyl, heteroaryl comprising 5 or 6 total ring atoms and 1-3 heteroatoms selected from N, O, and S, or a bicyclic ring comprising a heteroaryl ring having 5 or 6 total ring atoms and 1-3 heteroatoms selected from N, O, and S fused to cycloalkyl ring having 5 or 6 total ring atoms or a heterocycloalkyl ring having 5 or 6 total ring atoms and 1 or 2 heteroatoms selected from N, O, and S; wherein each of the phenyl, heteroaryl, and bicyclic rings is optionally substituted with 1-4 substituents;
each of R^{1a}, R^{1b}, and R² independently is H, D, halo, C₁₋₄alkyl, C₁₋₄haloalkyl, C₁₋₂alkylene-OH, C₀₋₂alkylene-C₁₋₄alkoxy, C₀₋₂alkylene-C₁₋₄haloalkoxy, C₀₋₂alkylene-CN, C₀₋₂alkylene-N(R^{N1})₂, C₁₋₂alkylene-heterocycloalkyl having 3-6 total ring atoms and 1 or 2 heteroatoms selected from N, O, and S, or R^{1b} and R², together with the carbon atoms to which they are attached, from a

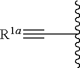

group;
each R³ independently is C₁₋₃alkyl, C₁₋₃haloalkyl, C₀₋₃alkyleneCN, C₀₋₃alkyleneOH, C₀₋₃alkylene-C₁₋₃alkoxy, oxo, spiro-cycloalkyl having 3-7 total ring atoms, spiro-heterocycloalkyl having 3-7 total ring atoms and 1 or 2 heteroatoms selected from N, O and S, or two adjacent R³, together with the atoms to which they are attached, form a fused cycloalkyl ring having 3-7 total ring atoms:
one R⁴ and R^{5a}, together with the atoms to which they are attached, form an optionally substituted ring having 6-10 total ring atoms and 0, 1, or 2 heteroatoms selected from N, O, and S, wherein the ring is saturated or unsaturated;
when n is 2, the other R⁴ is C₁₋₃alkyl, C₁₋₃haloalkyl, C₀₋₃alkyleneCN, C₁₋₂alkyleneOH, C₁₋₃alkylene-C₁₋₄alkoxy, oxo, spiro-cycloalkyl having 3-7 total ring atoms, or spiro-heterocycloalkyl having 4-7 total ring atoms and 1 or 2 heteroatoms selected from N, O and S;
R^{5b} is C₁₋₃haloalkyl, C₁₋₄alkyl, C₂₋₃alkenyl, C₂₋₃alkynyl, halo, C₁₋₃alkoxy, C₁₋₃thioalkoxy, cycloalkyl having 3-7 total ring atoms, cycloalkenyl having 5-7 total ring atoms, heterocycloalkyl having 3-7 total ring atoms and 1-3 heteroatoms selected from N, O, and S, or heterocycloalkenyl having 5-7 total ring atoms and 1-3 heteroatoms selected from N, O, and S, wherein each of foregoing is independently optionally substituted with 1-3 substituents, or R^{5a} and R^{5b}, together with the atoms to which they are attached, form a cycloalkyl ring having 3-7 total ring atoms;
each R⁶ independently is halo, CN, oxo, C₁₋₃alkyl, C₁₋₃haloalkyl, C₀₋₃alkyleneOH, C₀₋₃alkylene-C₁₋₃alkoxy, deuterated C₀₋₃alkylene-C₁₋₃alkoxy, C₁₋₄alkylene-N(R^{N1})₂, spiro-cycloalkyl having 3-7 total ring atoms, spiro-heterocycloalkyl having 4-7 total ring atoms and 1 or 2 heteroatoms selected from N, O and S; or two adjacent R⁶, together with the atoms to which they are attached, form a fused cycloalkyl ring having 3-7 total ring atoms; or Y and an adjacent R⁶, together with the atoms to which they are attached, form a fused cycloalkyl ring having 3-7 total ring atoms; wherein the fused cycloalkyl ring of any of the foregoing is optionally substituted with 1 or 2 substituents; or
   two non-adjacent Rejoin together to form a C₁₋₃alkylene bridge or a C₁₋₃ether bridge; and
each R^{N1} independently is H or C₁₋₃alkyl.

In some cases, R^{1a} is H or D. In some cases, R^{1a} is H. In some cases, R^{1a} is D. In some cases, R^{1b} is H or D. In some cases, R^{1b} is H. In some cases, R^{1b} is D. In some cases, R² is H or D. In some cases, R² is H. In some cases, R² is D. In some cases, at least one of R^{1a}, R^{1b}, and R² is H or D. In some cases, at least one of R A, R^{1b}, and R² is H. In some cases, at least one of R^{1a}, R¹, and R² is D. In some cases, at least two of R^{1a}, R^{1b}, and R² are H or D. In some cases, at least two of R^{1a}, R^{1b}, and R² are H. In some cases, at least two of R^{1a}, R^{1b}, and R² are D. In some cases, each of R^{1a}, R², and R² independently is H or D. In some cases, each of R^{1a}, R^{1b}, and R² independently is H. In some cases, each of R^{1a}, R^{1b}, and R² independently is D. In some cases, at least one of R^{1a}, R^{1b}, and R² is halo (e.g., Br, Cl, or F). In some cases, one of R^{1a}, R^{1b}, and R² is halo. In some cases, R^{1a} is halo and each of RI and R² is H. In some cases, at least one of R^{1a}, R^{1b}, and R² is Br, Cl, or F. In some cases, one of R^{1a}, R^{1b}, and R² is Br, Cl, or F. In some cases, R^{1a} is Br, Cl, or F and each of R^{1a} and R² is H. In some cases, at least one of R^{1a}, R^{1b}, and R² is Br or Cl. In some cases, one of R^{1a}, R^{1b}, and R² is Br or Cl. In some cases, R^{1a} is Br or Cl and each of R^{1b} and R² is H. In some cases, at least one of R^{1a}, R^{1b}, and R² is C₁₋₄alkyl or C₁₋₄haloalkyl. In some cases, one of R^{1a}, R^{1b}, and R² is C₁₋₆alkyl or C₁₋₄haloalkyl. In some cases, at least one of R^{1a}, R^{1b}, and R² is CH₃, CH₂CH₃, CH₂CH₂CH₃, CH(CH₃)₂, CH₂CH₂CH₂CH₃, CH₂F, CHF₂, or CF₃. In some cases, one of R^{1a}, R^{1b}, and R² is CH₃, CH—CH₃, CH₂CH₂CH₃, CH(CH₃)₂, CH₂CH₂CH₂CH₃, CH₂F, CHF₂, or CF₃. In some cases, at least one of R^{1a}, R^{1b}, and R² is CH₃ or CF₃. In some cases, one of R^{1a}, RID, and R² is CH, or CF₃. In some cases, at least one of R^{1a}, R^{1b}, and R² is C₁₋₂alkylene-OH, C₀₋₂alkylene-C₁₋₄alkoxy, C₀₋₂alkylene-C₁₄haloalkoxy, C₀₋₂alkylene-CN, or C₀₋₂alkylene-N(R^{N1})₂, and each R^{N1} independently is H or C₁₋₄alkyl. In some cases, each R^{N1} independently is H or CH₃. In some cases, each R^{N1} independently is H. In some cases, at least one of R^{1a}, R^{1b}, and R² is CH₂OH, OCH₃, CH₂OCH₃, OCF₃, CH₂OCF₃, CN, CH₂CN, NH₂, N(CH₃)₂, CH₂NH₂, or CH₂N(CH₃)₂. In some cases, one of R^{1a}, R^{1b}, and R² is CH₂OH, OCH₃, CH₂OCH₃, OCF₃, CH₂OCF₃, CN, CH₂CN, NH₂, N(CH₃)₂, CH₂NH₂, or CH₂N(CH₅)₂. In some cases, at least one of R^{1a}, R^{1b}, and R² is C₁₋₂alkylene-heterocycloalkyl wherein the heterocycloalkyl group contains 3-6 total ring atoms and 1 or 2 heteroatoms selected from N, O, and S. In some cases, the heterocycloalkyl is aziridinyl, oxiranyl, azetidinyl, oxetanyl, pyrrolidinyl, tetrahydrofuranyl, imidazolidinyl, pyrazolidinyl, oxathiolidinyl, isoxthiodinyl, oxazolidinyl, isoxazolidinyl, thiazolidinyl, isothiazolidinyl, piperidinyl, diazinyl, or morpholinyl. In some cases, the heterocycloalkyl is aziridinyl, azetidinyl, pyrrolidinyl, piperidinyl, or morpholinyl. In some cases, at least one of R^{1a}, R^{1b}, and R′ is aziridin-1-yl-methyl, azetidin-1-yl-methyl, pyrrolidine-1-yl-methyl, piperidin-1-yl-methyl, or morpholin-1-yl-methyl. In some cases, one of R^{1a}, R^{1b}, and R² is aziridin-1-yl-methyl, azetidin-1-yl-methyl, pyrrolidine-1-yl-methyl, piperidin-1-yl-methyl, or morpholin-1-yl-methyl. In some cases. In some R^{1b} and R² together with the carbon atoms to which they are attached from

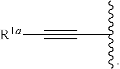

In some cases, R^{1a} is H. In some cases, R^{1b} and R² together with the carbon atoms to which they are attached from

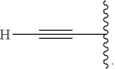 [SMILES: *C#C]

In some cases,

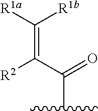

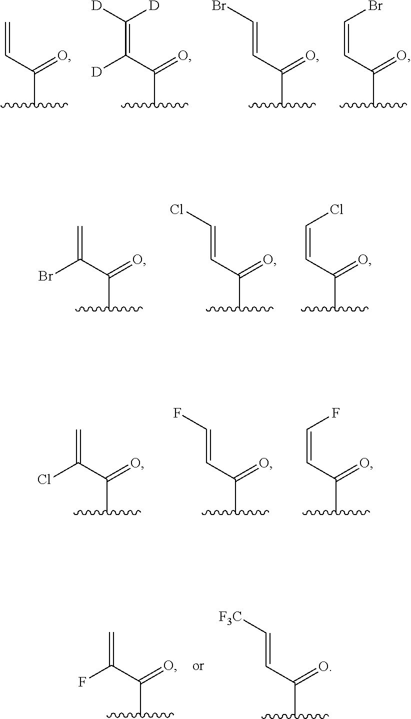

In some cases,

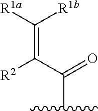

is

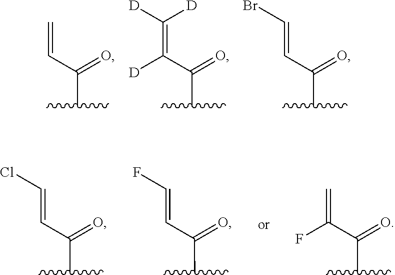

In some cases

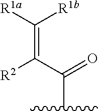

is

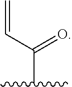 [SMILES: *C(=O)C=C]

In some cases, m is 0, and

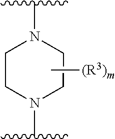

is

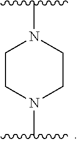 [SMILES: *N1CCN(*)CC1]

In some cases, m is 1. In some cases, m is 2. In some cases, m is 3. In some cases, m is 4. In some cases, at least one R³ is C₁₋₃alkyl or C₁₋₃haloalkyl. In some cases, at least one R³ is CH₃, CH₂CH₃, CH₂CH₂CH₃, CH(CH₃)₂, CF₃, CHF₂, or CH₂F. In some cases, at least one R³ is CH₃, CH₂CH₃, CF₃, CHF₂, or CH₂F. In some cases, m is 1 or 2 and each R³ is CH₃. In some cases, m is 1 and R³ is CF₃, CHF₂, or CH₂F. In some cases, at least one R³ is C₀₋₃alkyleneCN. In some cases, at least one R³ is CN or CH₂CN. In some cases, m is 1 and R³ is CN or CH₂CN. In some cases, at least one R³ is C₀₋₃alkyleneOH or C₀₋₃alkylene-C₁₋₃alkoxy. In some cases, at least one R³ is OH, CH₂OH, CH₂CH₂OH, OCH₃, CH₂OCH₃, or CH₂CH₂OCH₃. In some cases, m is 1 and R³ is OH. CH₂OH, CH₂CH₂OH, OCH₃, CH₂OCH₃, or CH₂CH₂OCH₃. In some cases, at least one R³ is oxo. In some cases, at least one R³ is spiro-cycloalkyl having 3-7 total ring atoms or spiro-heterocycloalkyl having 3-7 total ring atoms and 1 or 2 heteroatoms selected from N, O and S. In some cases, at least one R³ is spiro-cyclopropyl, spiro-cyclobutyl, spiro-cyclopentyl, spiro-azetidinyl, spiro-oxetanyl, spiro-pyrrolidinyl, spiro-imidazolidinyl, spiro-pyrazolidinyl, or spiro-tetrahydrofuranyl. In some cases, at least one R³ is spiro-cyclopropyl, spiro-cyclobutyl, spiro-oxetanyl, or spiro-tetrahydrofuranyl. In some cases, m is 1 and R³ is spiro-cyclopropyl or spiro-oxetanyl. In some cases, two adjacent R³, together with the atoms to which they are attached, form a fused cycloalkyl ring having 3-7 total ring atoms. In some cases, two adjacent R³, together with the atoms to which they are attached, form a fused-cyclopropyl ring, a fused-cyclobutyl ring, a fused-cyclopentyl ring, or a fused-cyclohexyl ring. In some cases, two adjacent R³, together with the atoms to which they are attached, form a fused-cyclopropyl ring or a fused-cyclobutyl ring. In some cases, each R³ independently is CH₃, CH₂CH₃, CF₃, CHF₂, CH₂F, CN, CH₂CN, OH, CH₂OH, CH₂CH₂OH, OCH₃, CH₂OCH₃, CH₂CH₂OCH₃, oxo, spiro-cyclopropyl, spiro-cyclobutyl, spiro-oxetanyl, or spiro-tetrahydrofuranyl. In some cases, m is 1 and R³ is CH₃, CF₃, CHF₂, CH₂F, CN, CH₂CN, CH₂OH, CH₂OCH₃, or spiro-oxetanyl. In some cases,

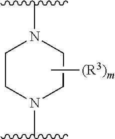

is

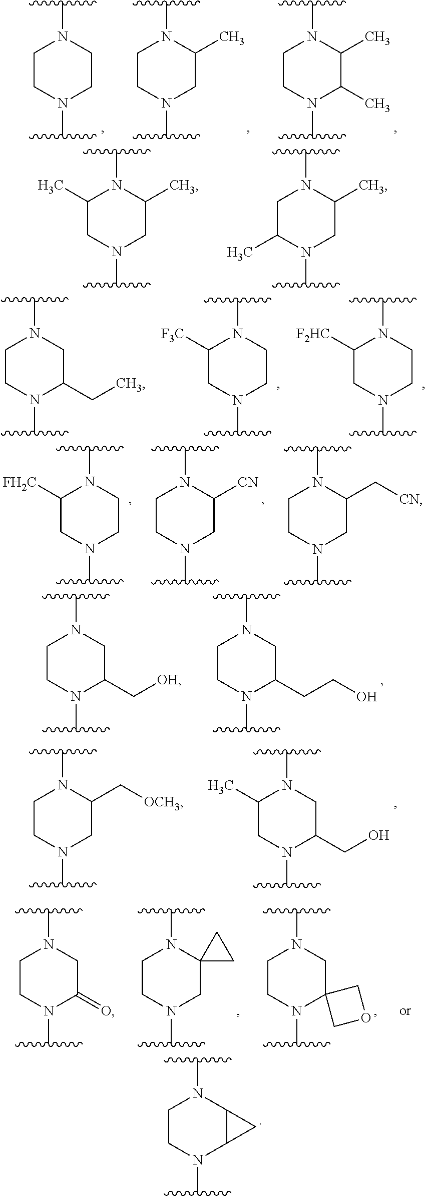

In some cases,

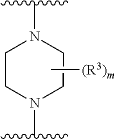

is

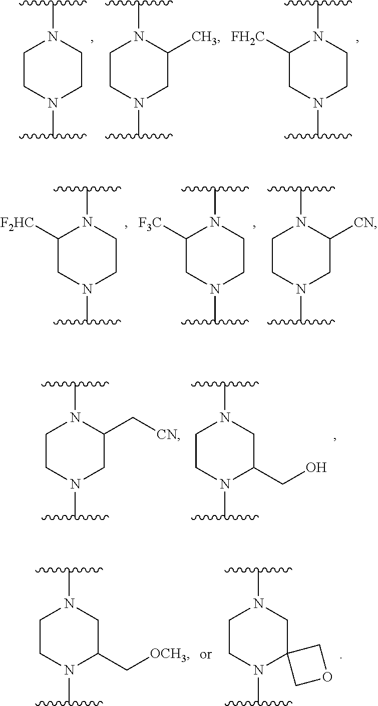

In some cases, A is N. In some cases, A is CH, C-halo, C—CN, C—C₁₋₃alkyl, C—C₁₋₃haloalkyl, C—C₀₋₃alkyleneOH, or C—C₀₋₃alkylene-C₁₋₃alkoxy. In some cases, A is CH. In some cases, A is C-halo or C—CN. In some cases, A is C—F or C—Cl. In some cases, A is C—F. In some cases, A is C—CN. In some cases, A is C—C₁₋₃alkyl or C—C₁₋₃haloalkyl. In some cases, A is C—CH₃, C—CH₂CH₃, C—CH₂CH₂CH₃, C—CH(CH₃)₂, C—CF₃, C—CHF₂, or C—CH₂F. In some cases, A is C—CH₃, C—CH₂F, C—CHF₂, or C—CF₃. In some cases, A is C—C₀₋₂alkyleneOH or C—C₀₋₃alkylene-C₁₋₄alkoxy. In some cases, A is C—OH, C—CH₂OH, C—CH₂CH₂OH, C—OCH₃, C—CH₂OCH₃, or C—CH₂CH₂OCH₃. In some cases, A is C—OH, C—CH₂OH, C—OCH₃, or C—CH₂OCH₃. In some cases, A is N, CH, C—F, C—Cl, C—CN, C—CH₃, C—CH₂CH₃, C—CHCH₂CH₃, C—CH(CH₃)₂, C—CF₃, C—CHF₂, C—CH₂F, C—OH, C—CH₂OH, C—CH₂CH₂OH, C—OCH₃, C—CH₂OCH₃, or C—CH₂CH₂OCH₃. In some cases, A is N, CH, C—F, C—Cl, C—CN, C—CH₃, C—CF₃, C—CHF₂, C—CH₂F, C—OH, C—CH₂OH, C—OCH₃, or C—CH₂OCH₃,

One R⁴ and R^{5a}, together with the atoms to which they are attached, form an optionally substituted ring having 6-10 total ring atoms and 0, 1, or 2 heteroatoms selected from N, O, and S. In some cases, the optionally substituted ring is saturated. In some cases, the optionally substituted ring is unsaturated. In some cases, the optionally substituted ring has 6 total ring atoms. In some cases, the optionally substituted ring has 7 total ring atoms. In some cases, the optionally substituted ring has 8 total ring atoms. In some cases, the optionally substituted ring has 9 or 10 total ring atoms. In some cases, the optionally substituted ring has 0 heteroatoms. In some cases, the optionally substituted ring has 1 or 2 heteroatoms selected from N, O, and S. In some cases, the optionally substituted ring has 1 or 2 oxygen atoms. In some cases, the optionally substituted ring is an ether. In some cases, the optionally substituted ring is a polyether. In some cases, the optionally substituted ring has 1 or 2 nitrogen atoms. In some cases, the ring is a cyclic amide (e.g., lactam) or a cyclic amine. In some cases, the ring is unsubstituted. In some cases, the ring is substituted with 1 or 2 substituents selected from the group consisting of C₁₋₃alkyl, C₁₋₃haloalkyl, oxo, halo, CN, C₀₋₃alkyleneOH, C₀₋₆alkylene-C₁₋₃alkoxy, cycloalkyl having 3-7 total ring atoms, cycloalkenyl having 5-7 total ring atoms, heterocycloalkyl having 4-7 total ring atoms and 1-3 heteroatoms selected from N, O, and S, heterocycloalkenyl having 5-7 total ring atoms and 1-3 heteroatoms selected from N, O, and S, and phenyl. In some cases, n is 1. In some cases, n is 2. In some cases, the other R⁴ is C₁₋₃alkyl or C₁₋₃haloalkyl. In some cases, the other R⁴ is CH₃, CH₂CH₃, CH₂CH₂CH₃, CH(CH₃)₂, CF₃, CHF₂, or CH₂F. In some cases, the other R⁵ is CH₃. In some cases, the other R⁴ is C₀₋₃alkyleneCN. In some cases, the other R⁴ is CN or CH₂CN. In some cases, the other R⁴ is C₁₋₂alkyleneOH or C₀₋₃alkylene-C₁₋₃alkoxy. In some cases, the other R⁴ is CH₂OH, CH₂CH₂OH, OCH₃, CH₂OCH₃, or CH₂CH₂OCH₃. In some cases, the other R⁴ is oxo. In some cases, the other R⁴ is spiro-cycloalkyl having 3-7 total ring atoms. In some cases, the other R⁴ is spiro-cyclopropyl, spiro-cyclobutyl, or spiro-cyclopentyl. In some cases, the other R⁴ is spiro-cyclopropyl or spiro-cyclobutyl. In some cases, the other R⁴ is spiro-heterocycloalkyl having 4-7 total ring atoms and 1 or 2 heteroatoms selected from N, O and S. In some cases, the other R⁴ is spiro-oxetanyl or spiro-tetrahydrofuranyl. In some cases, the other R⁴ is spiro-oxetanyl. In some cases, the other R⁴ is CH₃, CH₂CH₃, CH₂CH₂CH₃, CH(CH₃)₂, CF₃, CHF₂, CH₂F, CN, CH₂CN, CH₂OH, CH₂CH₂OH, OCH₃, CH₂OCH₃, CH₂CH₂OCH₃, oxo, spiro-cyclopropyl, spiro-cyclobutyl, or spiro-cyclopentyl. In some cases, the other R⁴ is CH₃, CH₂CH₃, CH₂CH₂CH₃, CH₂F, CN, CH₂CN, CH₂OH, CH₂CH₂OH, CH₂OCH₃, spiro-cyclopropyl, or spiro-oxetanyl. In some cases,

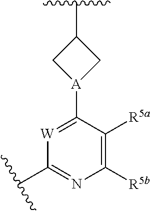

is

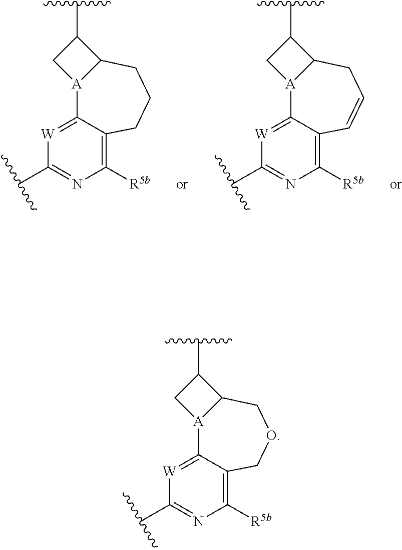

In some cases, W is CH. In some cases, W is C-halo (e.g., C—F, C—Cl, or C—Br) or C—CN. In some cases, W is C—F, C—Cl, or C—CN. In some cases, W is C—C₁₋₃alkyl or C—C₁.; haloalkyl. In some cases, W is C—CH₃, C—CH₂CH₃, C—CH₂CH₂CH₃, C—CH(CH₃)₂, C—CF., C—CHF₂, or C—CH₂F. In some cases, W is C—CH₃ or C—CH₂CH₃. In some cases, W is C—C₀₋₃alkyleneOH or C—C₀₋₃alkylene-C₁₋₄alkoxy. In some cases, W is C—OH, C—CH₂OH, C—CH₂CH₂OH, C—OCH₃, C—CH₂OCH₃, or C—CH₂CH₂OCH₃. In some cases, W is C—OH, C—CH₂OH, C—OCH₃, or C₂CH₂OCH₃. In some cases, W is CH, C—F, C—Cl, C—CN, C—CH₃, C—CH₂CH₃, C—CH₂CH₂CH₃, C—CH(CH₃)₂, C—CF₃, C—CHF₂, C—CH₂F, C—OH, C—CH₂OH, C—CH₂CH₂OH, C—OCH₃, C—CH₂OCH₃, or C₂CH₂CH₂OCH₃. In some cases, W is CH, C—F, C—Cl, C—CN, C—CH₃, C—CH₂CH₃, C—OH, C—CH₂OH, C—OCH₃, or C—CH₂OCH₃. In some cases, R^{5b} is C₁₋₃haloalkyl. In some cases, R^{5b} is CF₃, CF₂H, CFH₂, or CF₂CH₃. In some cases, R^{5b} is CF₃, CF₂H, or CFH₂. In some cases, R^{5b} is CF₃. In some cases, R^{5b} is CF₂H. In some cases, R^{5b} is CHF₂. In some cases, R^{5b} is halo. In some cases. R⁵ is Br, Cl, or F. In some cases, R^{5b} is C₁₋₃alkoxy or C₁₋₃thioalkoxy. In some cases, R⁵ is OCH₃, OCH₂CH₃, SCH₃, or SCH₂CH₃. In some cases, R⁵ is C₁₋₄alkyl, C₂₋₃alkenyl, or C₂₋₃alkynyl, wherein each of the alkyl, alkenyl, and alkynyl is optionally substituted with 1, 2, or 3 substituents independently selected from C₁₋₃alkyl, C₁₋₃haloalkyl, C₀₋₆alkylene (OH), C₀₋₆alkylene-C₁₋₃alkoxy, cycloalkyl having 3-7 total ring atoms, cycloalkenyl having 5-7 total ring atoms, heterocycloalkyl having 3-7 total ring atoms and 1-3 heteroatoms selected from N, O, and S, heterocycloalkenyl having 3-7 total ring atoms and 1-3 heteroatoms selected from N, O, and S, and phenyl. In some cases, the C₁₋₃alkyl, C₂₋₆alkenyl, and C₂₋₃alkynyl are unsubstituted. In some cases, the C₁₋₆alkyl, C₂₋₃alkenyl, and C₂₋₃alkynyl are substituted with 1, 2, or 3 substituents. In some cases, each of the 1, 2, or 3 substituents independently is selected from CH₃, CF₃, CF₂H, CFH₂, OH, OCH₃, OCF₃, CH₂OH, CH₂OCH₃, cyclopropyl, cyclobutyl, and phenyl. In some cases, R⁵ is cycloalkyl having 3-7 total ring atoms, cycloalkenyl having 5-7 total ring atoms, heterocycloalkyl having 3-7 total ring atoms and 1-3 heteroatoms selected from N, O, and S, or heterocycloalkenyl having 5-7 total ring atoms and 1-3 heteroatoms selected from N, O, and S, wherein each of the foregoing is optionally substituted with 1, 2, or 3 substituents independently selected from halo, C₁₋₃alkyl, C₁₋₃haloalkyl, C₀₋₆alkylene (OH), or C₀₋₃alkylene-C₁₋₃alkoxy. In some cases, R⁵ is cyclopropyl, cyclobutyl, cyclopentenyl, oxetanyl, or tetrahydrofuranyl. In some cases, R^{5b} is CH₃, CH₂CH₃, CH₂CH₂CH₃, CH(CH₃)₂,

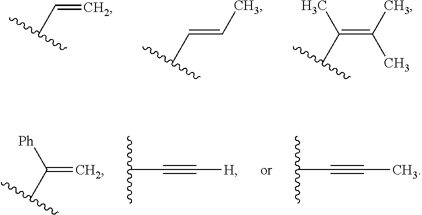

In some cases, R^{5b} is CH₃, CH₂CH₃, CH₂CH—CH₃, or CH(CH₃)₂.

In some cases, X is

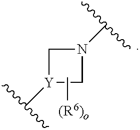

In some cases, X is

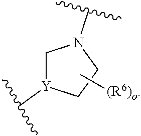

In some cases, X is

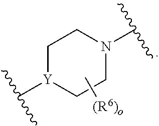

In some cases, X is

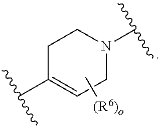

In some cases, X is

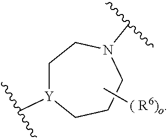

In some cases, Y is N. In some cases, Y is C—H. In some cases, Y is C-halo, C—CN, C—C₁₋₃alkyl, C—C₁₋₃haloalkyl, C—C₀₋₃alkyleneOH, or C—C₀₋₃alkylene-C₁₋₄alkoxy. In some cases, Y is C—F, C—Cl, or C—CN. In some cases, Y is C—C₁₋₃alkyl, C—C₁₋₃haloalkyl. In some cases, Y is C—CH₃, C—CH₂CH₃, C—CH₂F, C—CHF₂, or C—CF₃. In some cases, Y is C—C₀₋₂alkyleneOH or C—C₀₋₃alkylene-C₁₋₄alkoxy. In some cases, Y is C—OH, C—CH₂OH, C—OCH₃, or C—CH₂OCH₃. In some cases, o is 0. In some cases, o is 1. In some cases, o is 2. In some cases, o is 3. In some cases, o is 4. In some cases, at least one R⁶ is halo or CN. In some cases, at least one R⁶ is Br, Cl, F, or CN. In some cases, at least one R⁶ is oxo. In some cases, o is 1 or 2 and each R⁶ independently is F. In some cases, at least one R⁶ is C₁₋₆alkyl or C₁₋₃haloalkyl. In some cases, at least one R⁶ is CH₃, CH₂F, CHF₂, or CF₃. In some cases, o is 1 or 2 and each R⁶ independently is CH₃. In some cases, at least one R′ is C₀₋₃alkyleneOH, C₀₋₆alkylene-C₁₋₃alkoxy, deuterated C₀₋₆alkylene-C₁₋₃alkoxy, or C₁₋₄alkylene-N(R^{N1})₂, and each R^{N1} independently is H or CH₃. In some cases, each R^{N1} independently is H. In some cases, at least one R⁶ is OH, CH₂OH. CH₂CH₂OH, OCH₃, OCD₃, or CH₂OCH₃, or CH₂CH₂OCH₃, In some cases, o is 1 and R⁶ is OH, CH₂OH, OCH₃, or CH₂OCH₃. In some cases, at least one R⁶ is spiro-cycloalkyl having 3-7 total ring atoms or spiro-heterocycloalkyl having 4-7 total ring atoms and 1 or 2 heteroatoms selected from N, O and S. In some cases, at least one R⁶ is spiro-cyclopropyl, spiro-cyclobutyl, spiro-oxetanyl, or spiro-tetrahydrofuranyl. In some cases, o is 1 and R⁶ is spiro-cyclopropyl. In some cases, two adjacent R⁶, together with the atoms to which they are attached, form a fused cycloalkyl ring having 3-7 total ring atoms. In some cases, Y and an adjacent R⁶, together with the atoms to which they are attached, form a fused cycloalkyl ring having 3-7 total ring atoms, wherein the fused cycloalkyl ring of any of the foregoing is optionally substituted with 1 or 2 substituents selected from halo, OH, C₁₋₄alkoxy, or CN. In some cases, the fused cycloalkyl ring of any of the foregoing is fused-cyclopropyl, fused-cyclobutyl, or fused-cyclopentyl. In some cases, two non-adjacent R⁶ join together to form a C₁₋₂alkylene bridge or a C₁₋₃ether bridge. In some cases, two non-adjacent R⁶ join together to form a C₁₋₄alkylene bridge. In some cases, two non-adjacent R⁶ join together to form a C₂alkylene bridge. In some cases, two non-adjacent R⁶ join together to form a C₃alkylene bridge. In some cases, two non-adjacent R⁶ join together to form a C₁₋₃ether bridge

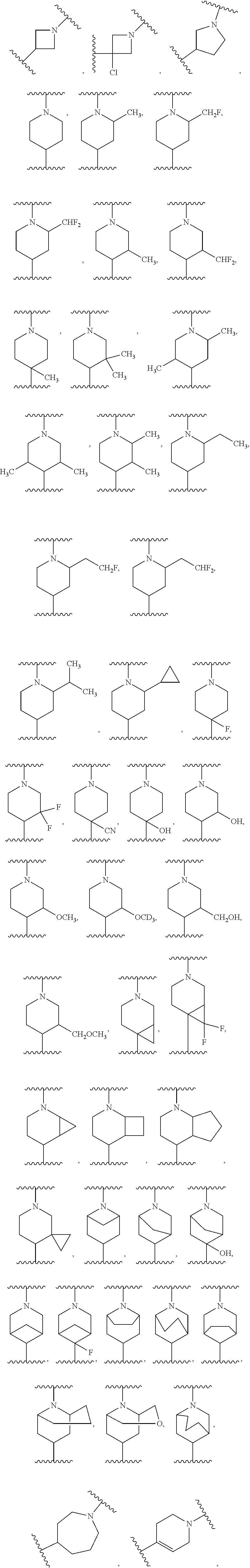

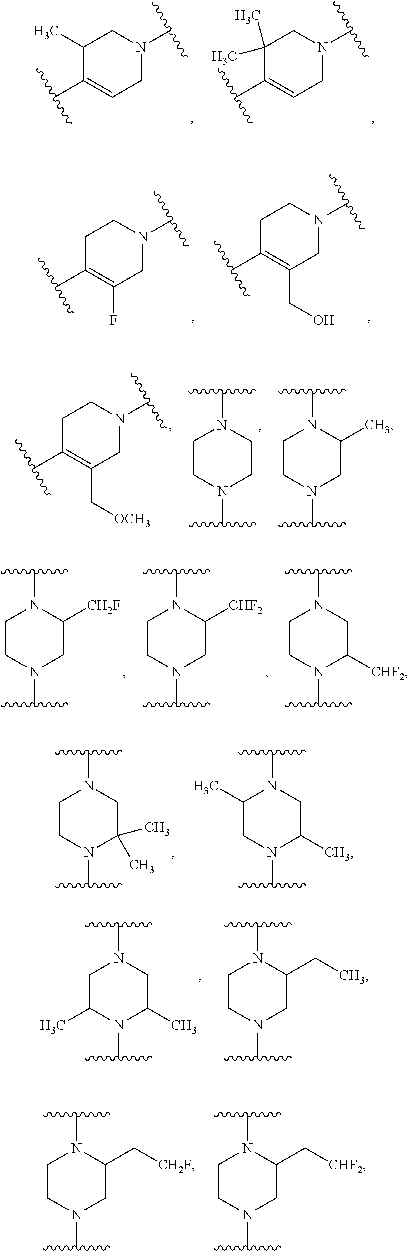

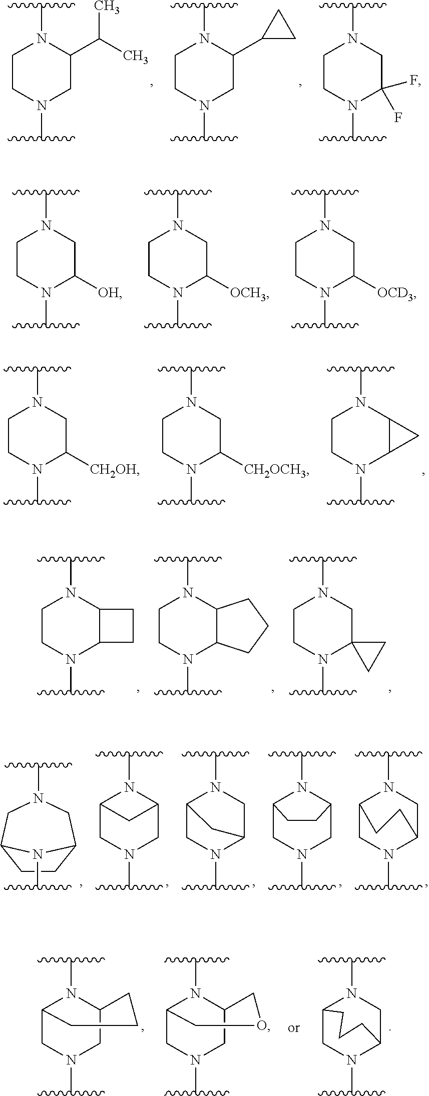

In some cases. X is

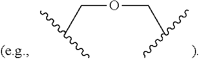 [SMILES: *COC*]

In some cases, X is

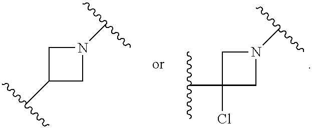 [SMILES: *C1CN(*)C1 ; *N1CC(*)(Cl)C1]

In some cases, X

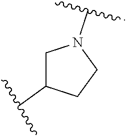 [SMILES: *C1CCN(*)C1]

In some cases, X is

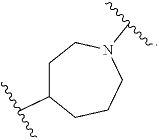 [SMILES: *C1CCCN(*)CC1]

In some cases, X is

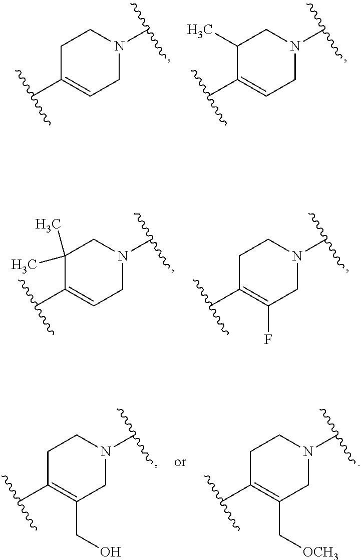

In some cases, X is

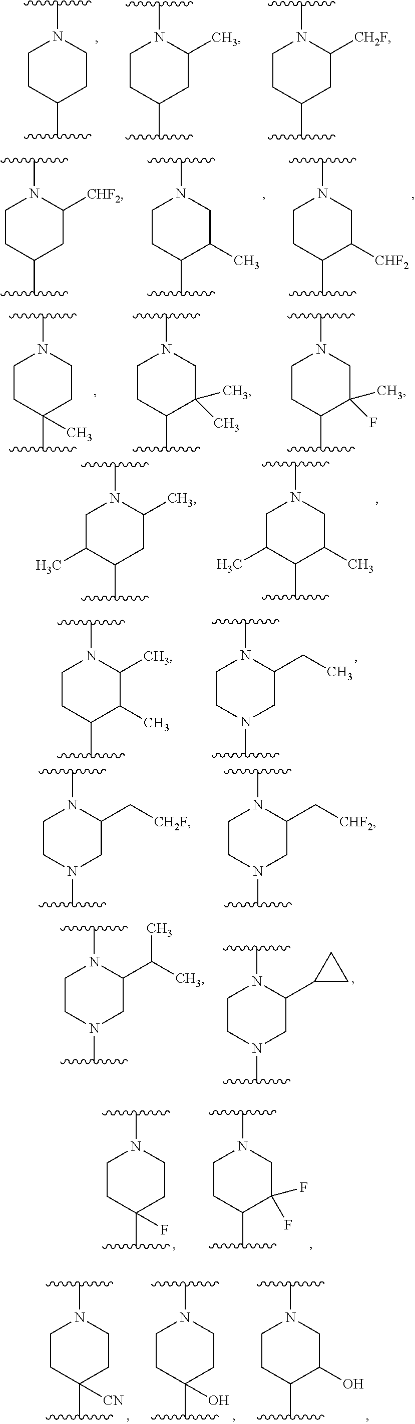

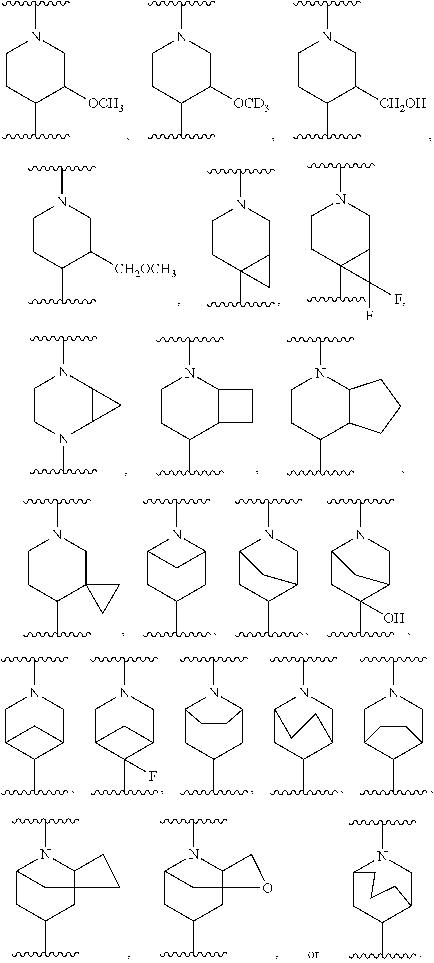

In some cases, X is

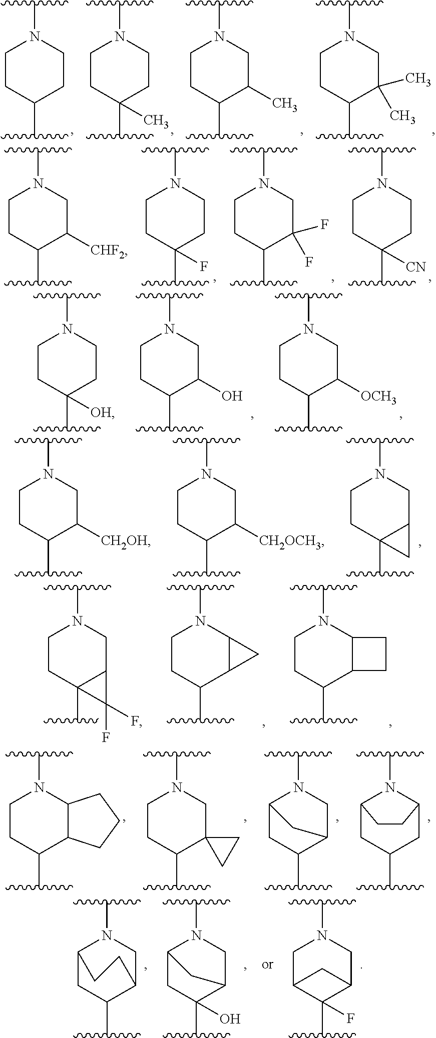

In some cases, X is

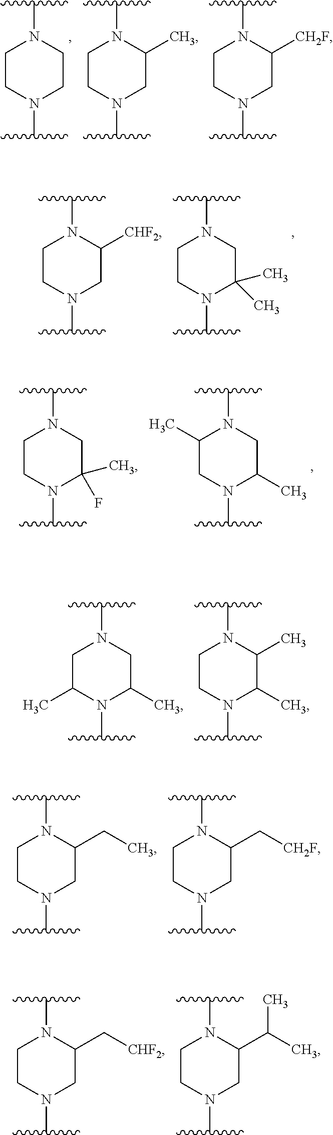

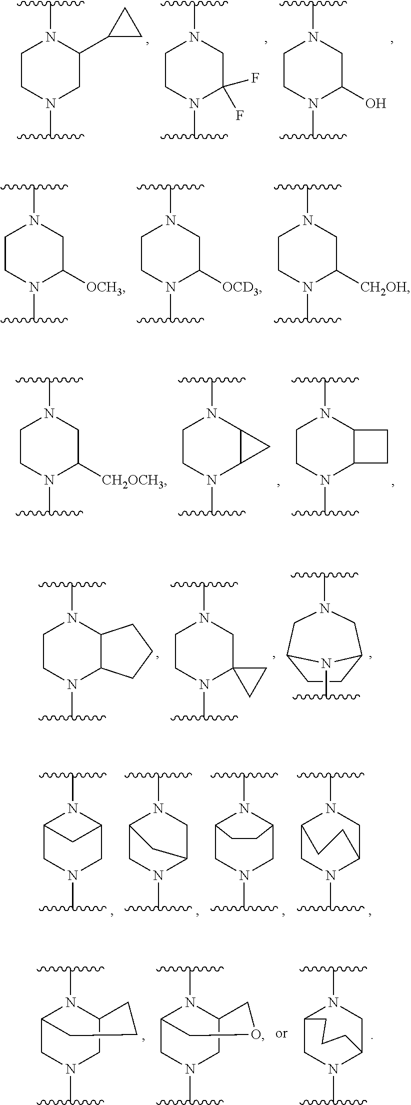

In some cases, X is

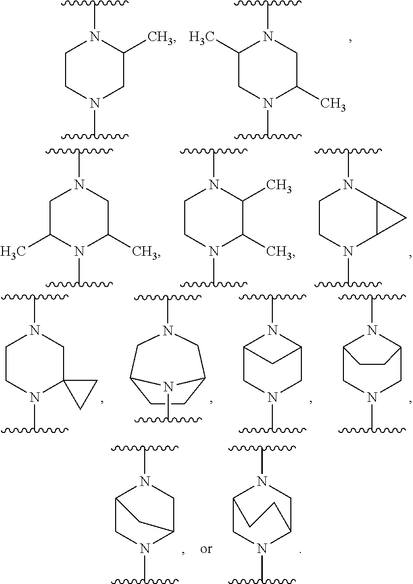

In some cases, X is

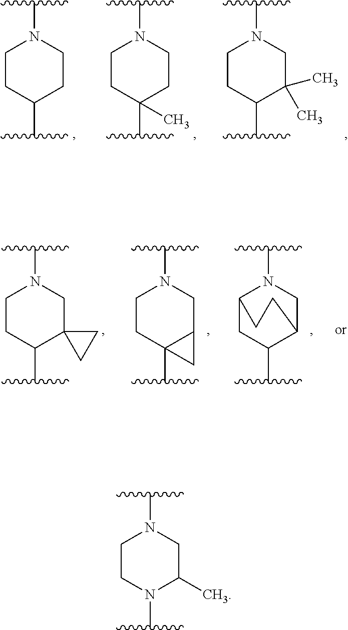

In some cases, X is

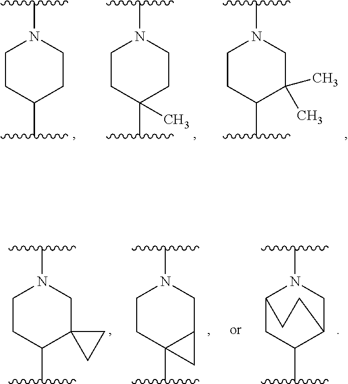

In some cases, X is

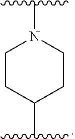 [SMILES: *C1CCN(*)CC1]

In some cases, X is

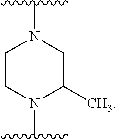 [SMILES: *N1CCN(*)C(C)C1]

In some cases, Z is phenyl optionally substituted with 1-4 substituents selected from halo, C₀₋₃alkyleneCN, C₀₋₃alkyleneOH, C₀₋₆alkylene-C₁₋₄alkoxy, C₀₋₃alkylene-C₁₋₄thioalkoxy, and

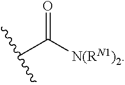

In some cases, each R^{N1} independently is H or CH₃. In some cases, each R^{N1} independently is H. In some cases, the 1-4 substituents are selected from F, Cl, CN, OCH₃, SCH₃, CH₂OH, and

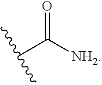 [SMILES: *C(N)=O]

In some cases, Z is

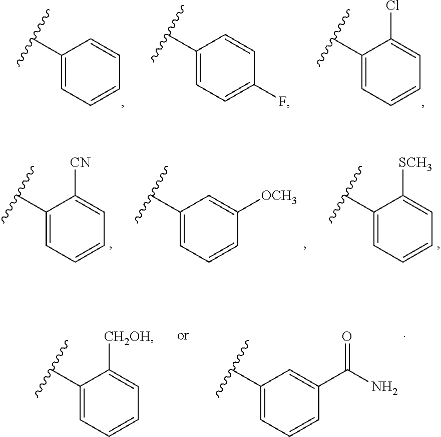

In some cases, Z is

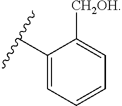 [SMILES: *c1ccccc1CO]

In some cases, Z is heteroaryl comprising 5 or 6 total ring atoms and 1-3 heteroatoms selected from N, O, and S. In some cases, the heteroaryl comprises 5 total ring atoms. In some cases, the heteroaryl comprises 6 total ring atoms. In some cases, the heteroaryl is optionally substituted with 1-4 substituents. In some cases, the heteroaryl is pyrrolyl, furanyl, thiophenyl, pyrazolyl, imidazolyl, thiazolyl, isothiazolyl, oxazolyl, isoxazolyl, triazolyl, thiadiazolyl, oxadiazolyl, pyridyl, pyridazinyl, pyrimidinyl, pyrazinyl, or triazinyl. In some cases, the heteroaryl is pyrrolyl, furanyl, thiophenyl, pyrazolyl, imidazolyl, thiazolyl, isothiazolyl, oxazolyl, isoxazolyl, triazolyl, thiadiazolyl, oxadiazolyl, or triazolyl. In some cases, the heteroaryl is pyrazolyl, imidazolyl, thiazolyl, isothiazolyl, oxazolyl, isoxazolyl, or triazolyl. In some cases, the heteroaryl is pyrazolyl, imidazolyl, thiazolyl, or isothiazolyl. In some cases, the heteroaryl is pyrazolyl. In some cases, the heteroaryl is imidazolyl. In some cases, the heteroaryl is thiazolyl. In some cases, the heteroaryl is isothiazolyl. In some cases, the heteroaryl is pyridyl, pyridazinyl, pyrimidinyl, or pyrazinyl. In some cases, the heteroaryl pyridyl.

In some cases, the heteroaryl is unsubstituted. In some cases, the heteroaryl is substituted with 1-4 substituents. In some cases, the heteroaryl is substituted with 1 or 2 substituents. In some cases, the heteroaryl is substituted with 3 or 4 substituents. In some cases, the heteroaryl is substituted with 1 substituent. In some cases, the heteroaryl is substituted with 2 substituents. In some cases, the heteroaryl is substituted with 3 substituents. In some cases, the heteroaryl is substituted with 4 substituents. In some cases, each of the 1-4 substituents independently is selected from the group consisting of halo (e.g, Br, Cl, or F), CN, C₁₋₆alkyl, C₁₋₆haloalkyl, C₂₋₆alkenyl, C₀₋₃alkylene-OH, C₀₋₃alkylene-C₁₋₃alkoxy, C₀₋₆alkylene-N(R^{N1}), wherein each RN′ independently is H or C₁₋₃alkyl, C₀₋₂alkylene-cycloalkyl having 3-6 total ring atoms, C₀₋₂alkylene-heterocycloalkyl having 5 or 6 total ring atoms and 1-3 heteroatoms selected from N, O, and S, and C₀₋₂alkylene-phenyl. In some cases, the heteroaryl is substituted with C₀₋₂alkylene-heterocycloalkyl having 3-6 total ring atoms and 1-3 heteroatoms selected from N, O, and S. Optionally, the alkyl, alkenyl, C₀₋₆alkylene-C₁₋₃alkoxy, cycloalkyl, heterocycloalkyl, and phenyl substituents are each independently substituted with 1-3 substituents selected from deuterium, halo (e.g., Br, Cl, or F), OH, CH₃, OCH₃, and OCD₃. In some cases, the C₁₋₃alkyl is unsubstituted. In some cases, the C₁₋₆alkyl is CH₃, CH₂CH₃, CH₂CH₂CH₃, or CH(CH₃)₂. In some cases, the C₁₋₆alkyl is substituted with 1-3 substituents selected from deuterium, halo, OH, OCH₃, and OCD₃. In some cases, the substituted C₁₋₆alkyl is CD₃, CH(CH₃)CH₂OH, C(CH₃)₂OH, C(CH₃)₂CH₂OH, CH₂C(CH₃)₂OH, CH(CH₃)CH₂OCH₃, C(CH₃)₂CH₂OCH₃. CH₂CH(CH₃)OCH₃, CH₂C(CH₃)₂OCH₃, CH(CH₃)CH₂OCD₃, C(CH₃)₂CH₂OCD₃, CH₂CH(CH₃)OCD₃, or CH₂C(CH₃)₂OCD₃, In some cases, the C₁₋₆haloalkyl is CF₃, CHF₂, CH₂F, CH₂CHF₂. CH₂CH₂F, CH(CH₂F)₂, CH(CH₃)CH₂F, or CH(CH₃)CHF₂. In some cases, the C₂₋₆alkenyl is unsubstituted. In some cases, the C₂₋₆alkyl is CH═CH₂, CH₂CH═CH₂, or CH═CHCH₃. In some cases, the C₂₋₆alkyl is substituted with 1-3 substituents selected from deuterium, halo, OH, OCH₃, and OCD₃. In some cases, the C₂₋₆haloalkenyl is C(═CH₂)CH₂F. In some cases, the C₀₋₆alkylene-OH is OH, CH₂OH, or CH₂CH₂OH. In some cases, the optionally substituted C₀₋₃alkylene-C₁₋₃alkoxy is OCH₃, OCD₃, CH₂OCH₃, CH₂OCD₃, CH₂CH₂OCH₃, CH₂CH₂OCD₃, CHFCH₂OCH₃, CF₂CH₂OCH₃, or CH₂CH₂CH₂OCH₃, or CH₂CH₂CH₂OCD₃. In some cases, C₀₋₆alkylene-N(R^{N1}), is NH₂, CH₂NH₂, CH₂NHCH₃, CH₂N(CH₃)₂, CH₂CH₂NH₃, CH₂CH₂NHCH₃, or CH₂CH₂N(CH₃)₂. In some cases, the cycloalkyl of the optionally substituted C₀₋₂alkylene-cycloalkyl is cyclopropyl, cyclobutyl, cyclopentyl, or cyclohexyl. In some cases, the C₀₋₂alkylene-cycloalkyl is unsubstituted. In some cases, the C₀₋₂alkylene-cycloalkyl is substituted with 1-3 substituents each independently selected from halo (e.g., Br, Cl, or F), OH, CH₃, OCH₃, and OCD₃. In some cases, the optionally substituted C₀₋₂alkylene-cycloalkyl is

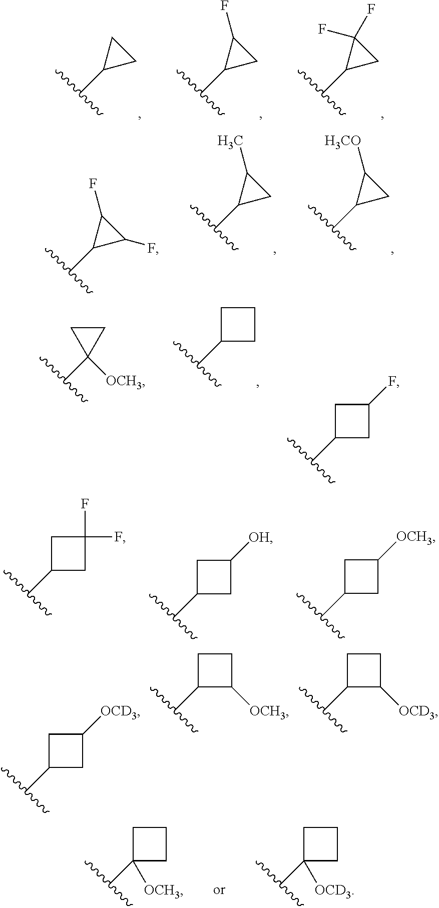

In some cases, the heterocycloalkyl of the optionally substituted C₀₋₂alkylene-heterocycloalkyl is azetidinyl, oxetanyl, pyrrolidinyl, pyrazolidinyl, tetrahydrofuranyl, tetrahydropyranyl, or piperidinyl. In some cases, the C₀₋₂alkylene-heterocycloalkyl is unsubstituted. In some cases, the C₀₋₂alkylene-heterocycloalkyl is substituted with 1-3 substituents each independently selected from halo (e.g., Br, Cl, or F), OH, CH₃, OCH₃, and OCD; In some cases, the C₀₋₂alkylene-heterocycloalkyl is

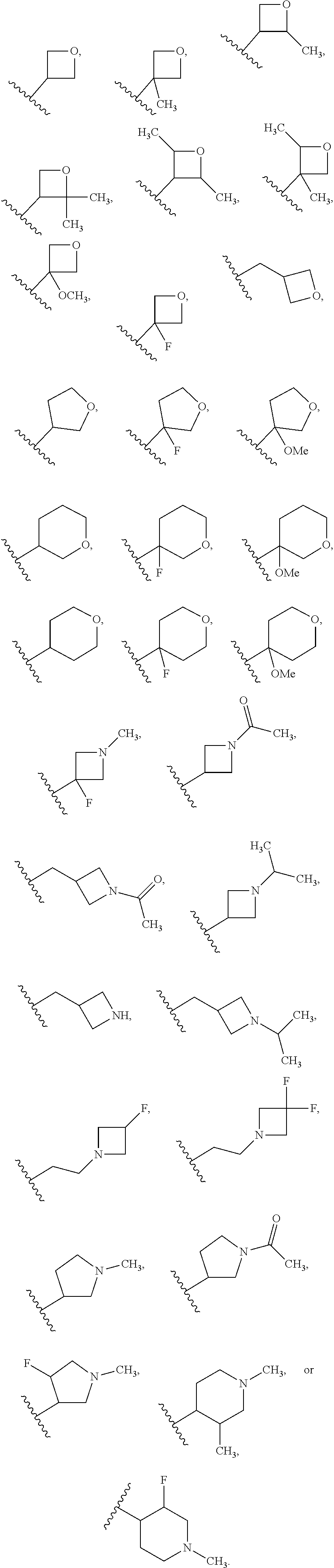

In some cases, Z is heteroaryl comprising 5 or 6 total ring atoms and 1-3 heteroatoms selected from N, O, and S, and each of the 1-4 substituents of the heteroaryl is independently selected from the group consisting of Cl, F, CN, CH₃, CD₃, CH₂CH₃, CH(CH₃)₂, CF₃, CHF₂, CH₂F, CH₂CHF₂, CH₂CH₂F, CH(CH₂F)₂, CH(CH₃)CH₂F, CH(CH₃)CHF₂, C(═CH₂)CH₂F, OH, CH₂OH, CH₂CH₂OH, CH(CH₃)CH₂OH, C(CH₃)₂OH, C(CH₃)₂CH₂OH, CH₂C(CH₃)₂OH, OCH₃, OCD₃, CH₂OCH₃, CH₂OCD₃, CH₂CH₂OCH₃, CHFCH₂OCH₃, CF₂CH₂OCH₃, CH₂CH₂OCD₃, CH₂CH₂OCH₂CH₃,CH₂CH₂CH₂OCH₃, CH₂CH₂CH₂OCD₃, CH(CH₃)CH₂OCH₃, CH(CH₃)CH₂OCD₃,C(CH₃)₂CH₂OCH₃, C(CH₃)₂CH₂OCD₃, CH₂CH(CH₃)OCH₃, CH₂CH(CH₃)OCD₃, CH₂C(CH₃)₂OCH₃, CH₂C(CH₃)₂OCD₃, NH₂, CH₂NH₂, CH₂NHCH₃, CH₂N(CH₃)₂, CH₂CH₂NH₂, CH₂CH₂NHCH₃, CH₂CH₂N(CH₃)₂,

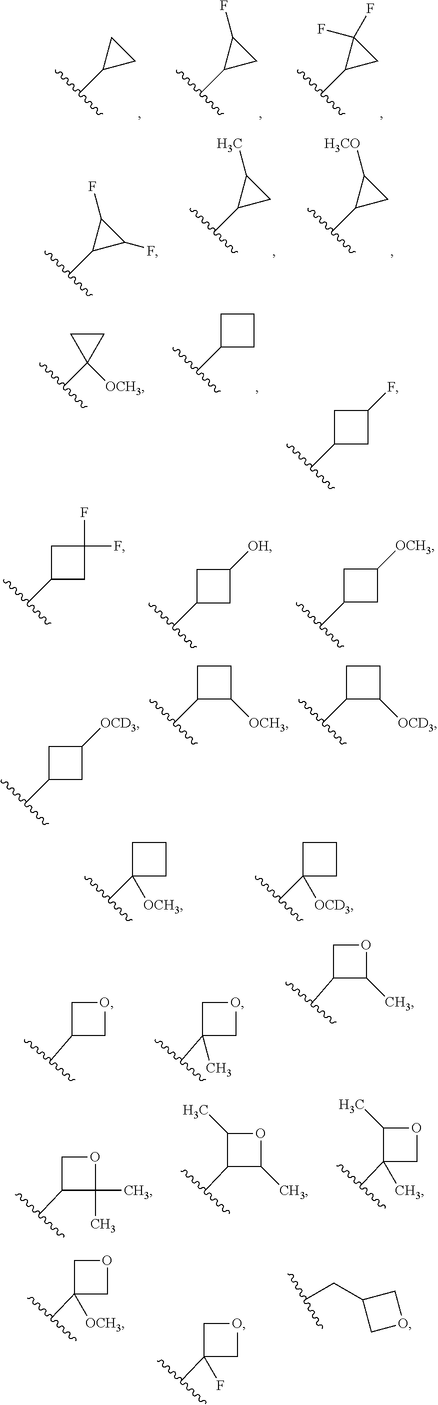

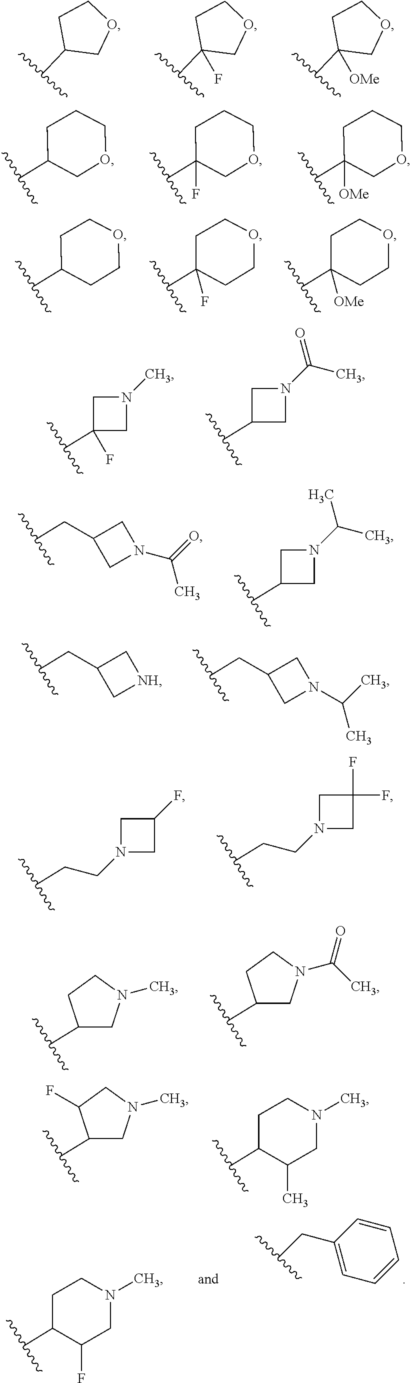

In some cases, Z is heteroaryl comprising 5 or 6 total ring atoms and 1-3 heteroatoms selected from N, O, and S, and each of the 1-4 substituents of the heteroaryl independently is CH₃, CH(CH₃)₂, C(CH₃)₂OH, CH₂OCD₃, CH₂CH₂OCH₃, CF₂CH₂OCH₃, CH₂CH₂OCD₃, CH₂CH₂CH₂OCH₃, CH₂CH(CH₃)OCH₃, CH₂C(CH₃)₂OCH₃,

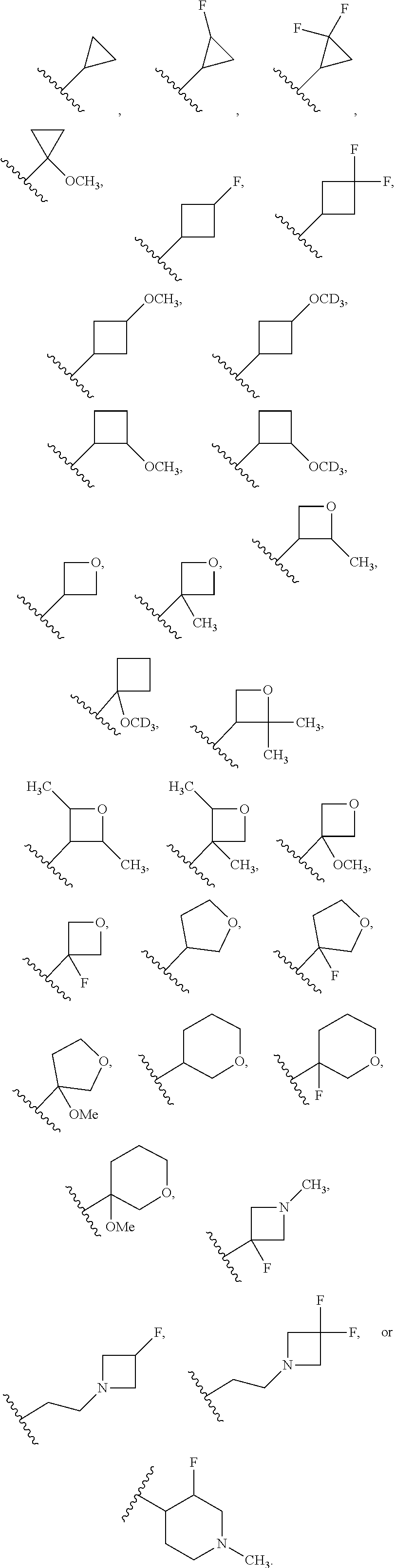

In some cases, Z is heteroaryl comprising 5 or 6 total ring atoms and 1-3 heteroatoms selected from N, O, and S, and each of the 1-4 substituents of the heteroaryl independently is CH₃, CH₂CH₂OCH₃, CF₂CH₂OCH₃, CH₂CH₂OCD₃, CH₂CH₂CH₂OCH₃, CH₂CH(CH₃)OCH₃, CH₂C(CH₃)₂OCH₃,

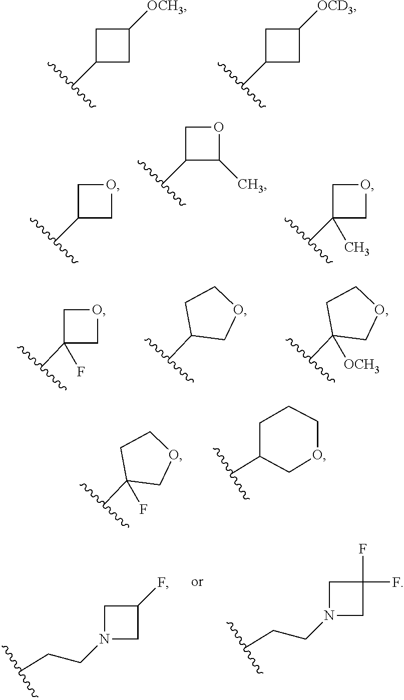

In some cases, the heteroaryl group has 2 substituents selected from CH₃, CH₂CH₂OCH₃,

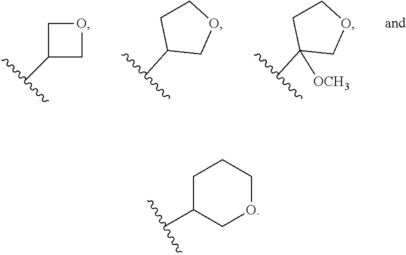 [SMILES: *C1COC1 ; *C1CCOC1 ; *C1(OC)CCOC1 ; *C1CCCOC1]

In some cases, Z is

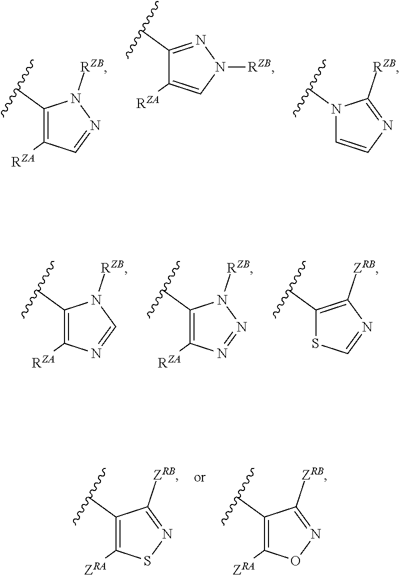

wherein R^{ZA} and R^{ZB} the same as previously defined for the 1-4 substituents of the heteroaryl group of Z. In some cases, Z is

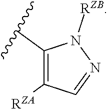

In some cases, Z is

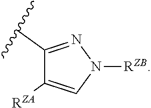

In some cases, Z is

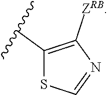

In some cases, Z is

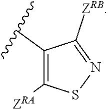

In some cases, each of R^{ZA} and R^{ZB} is selected from Cl, F. CN, CH₃, CD₃, CH₂CH₃, CH(CH₃)₂, CF₃, CHF₂, CH₂F, CH₂CHF₂, CH₂CH₂F, CH(CH₂F)₂, CH(CH₃)CH₂F, CH(CH₃)CHF₂, C(═CH₂)CH₂F, OH, CH₂OH, CH₂CH₂OH, CH(CH₃)CH₂OH, C(CH₃)₂OH, C(CH₃)₂CH₂OH, CH₂C(CH₃)₂OH, OCH₃, OCD₃, CH₂OCH₃, CH₂OCD₃, CH₂CH₂OCH₃, CHFCH₂OCH₃, CF₂CH₂OCH₃, CH₂CH₂OCD₃, CH₂CH₂OCH₂CH₃,CH₂CH₂CH₂OCH₃, CH₂CH₂CH₂OCD₃, CH(CH₃)CH₂OCH₃, CH(CH₃)CH₂OCD₃,C(CH₃)₂CH₂OCH₃, C(CH₃)₂CH₂OCD₃, CH₂CH(CH₃)OCH₃, CH₂CH(CH₃)OCD₃, CH₂C(CH₃)₂OCH₃, CH₂C(CH₃),OCD₃, NH₂, CH₂NH₂, CH₂NHCH₃, CH₂N(CH₃)₂, CH₂CH₂NH), CH₂CH₂NHCH₃, CH₂CH₂N(CH₃)₂,

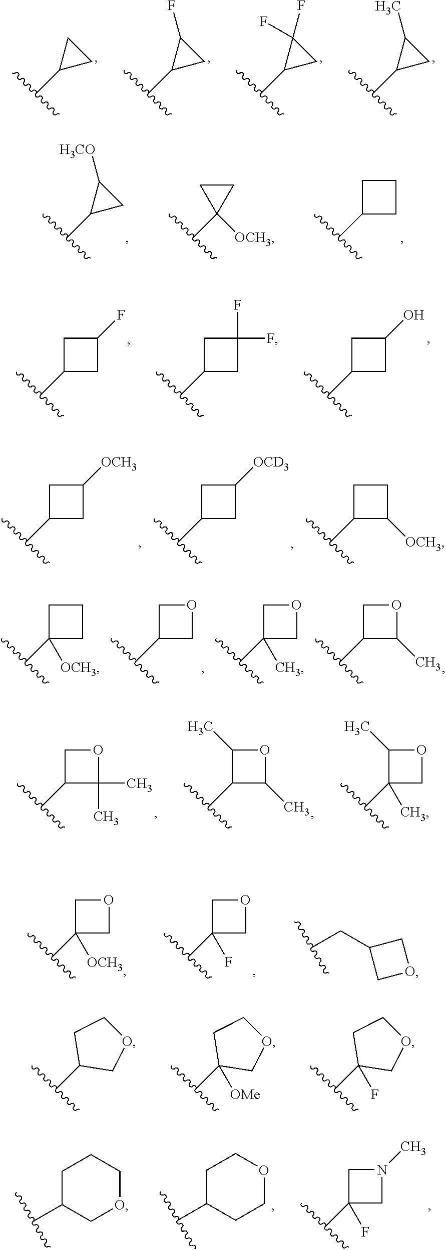

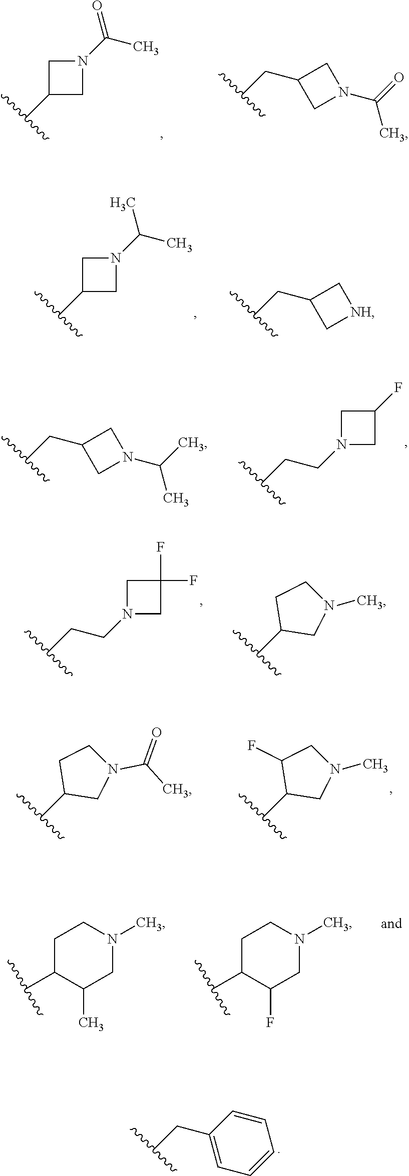

In some cases, R^{ZA} is Cl, F, CH₃, CH₂CH₃, CH(CH₃)), CF₃, CHF₂, CH₂F, CH₂CHF₂, or CH₂CH₂F; and R^{ZB} is CH₃, CH₂CH₂OCH₃, CF₂CH₂OCH₃, CH₂CH₂OCD₃, CH₂CH₂CH₂OCH₃, CH₂CH(CH₃)OCH₃, CH₂C(CH₃)₂OCH₃,

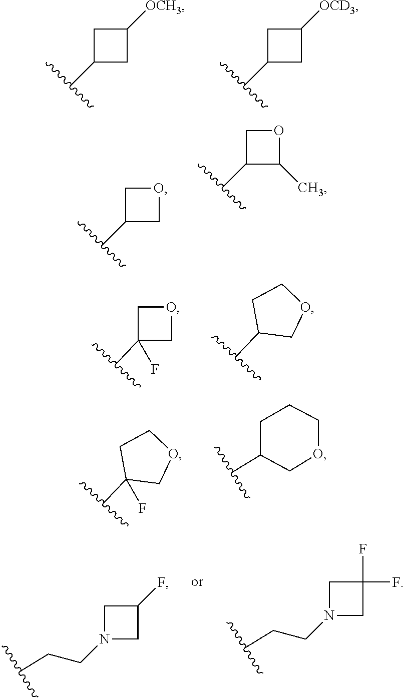

In some cases, R^{ZA} is CH₃; and R^{ZB} is CH₃, CH—CH₂OCH₃, CF₂CH₂OCH₃, CH—CH₂OCD₃, CH₂CH₂CH₂OCH₃, CH₂CH(CH₃)OCH₃, CH₂C(CH₃)₂OCH₃,

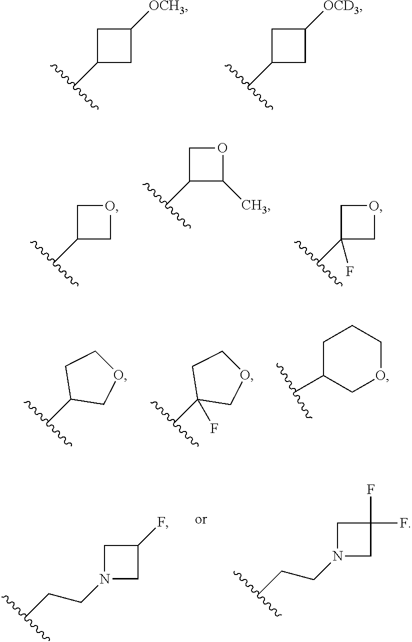

In some cases, Z is

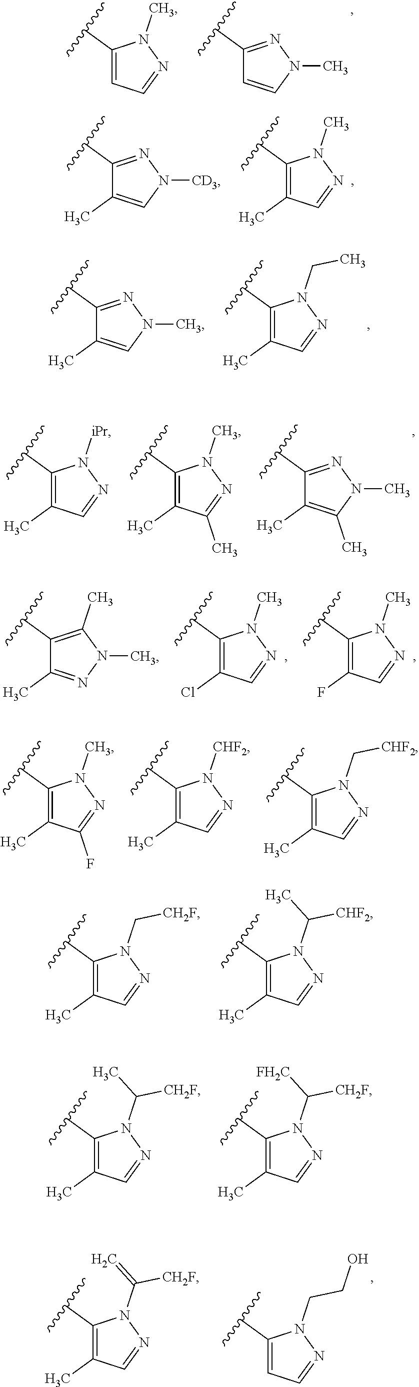

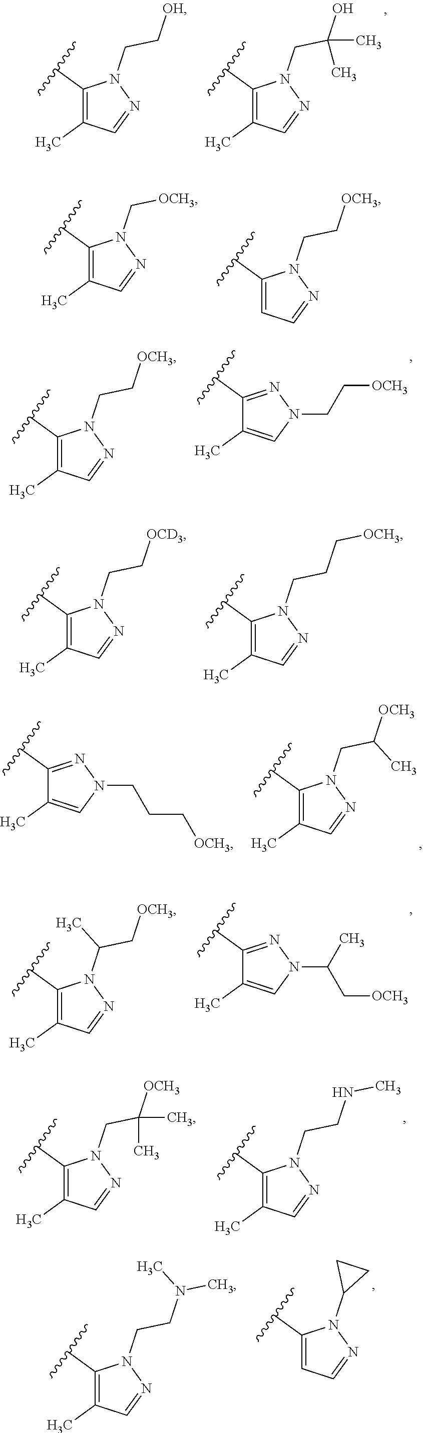

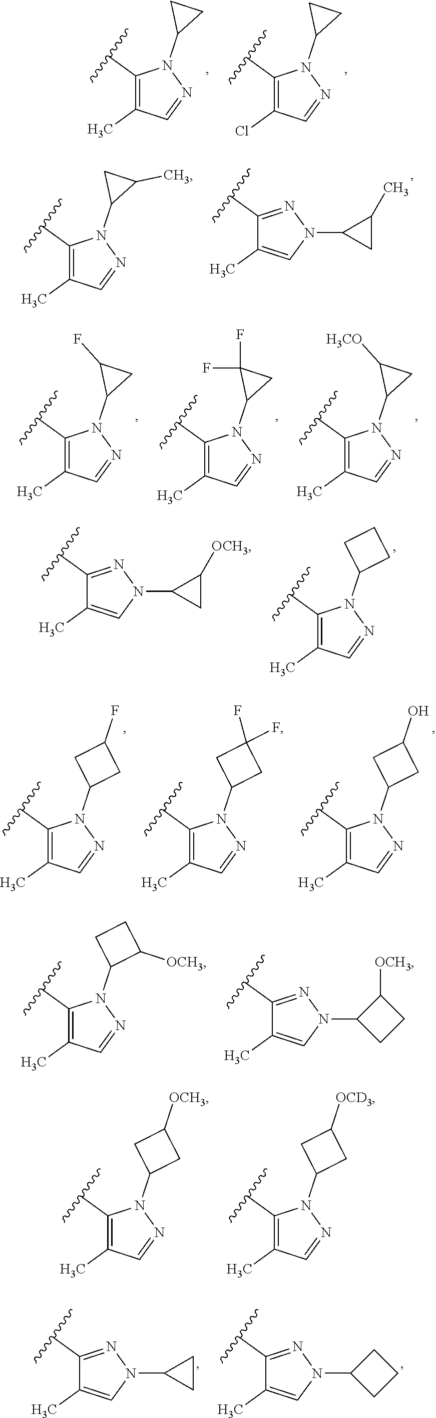

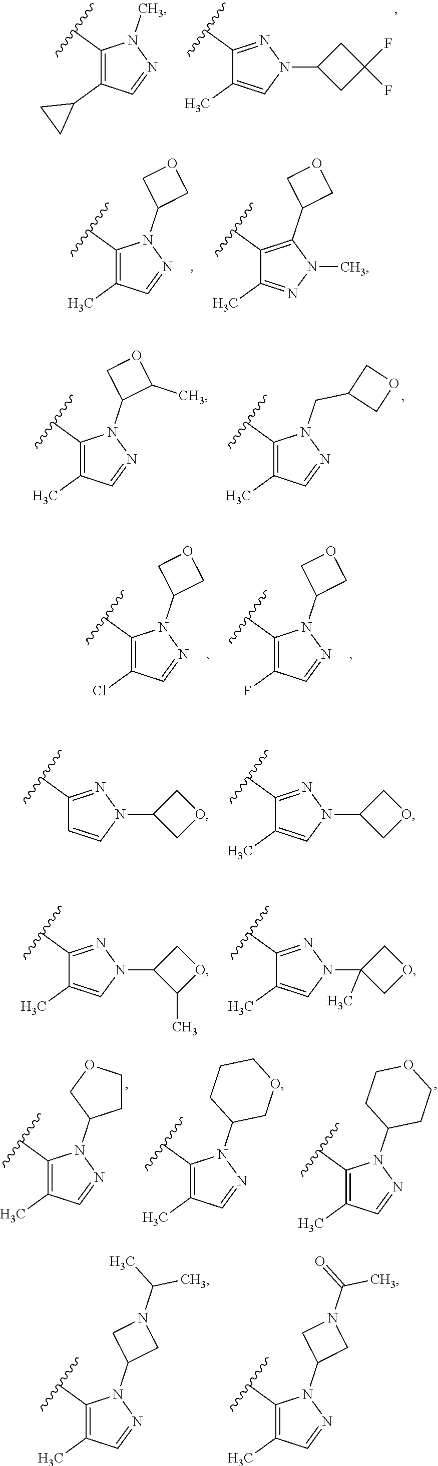

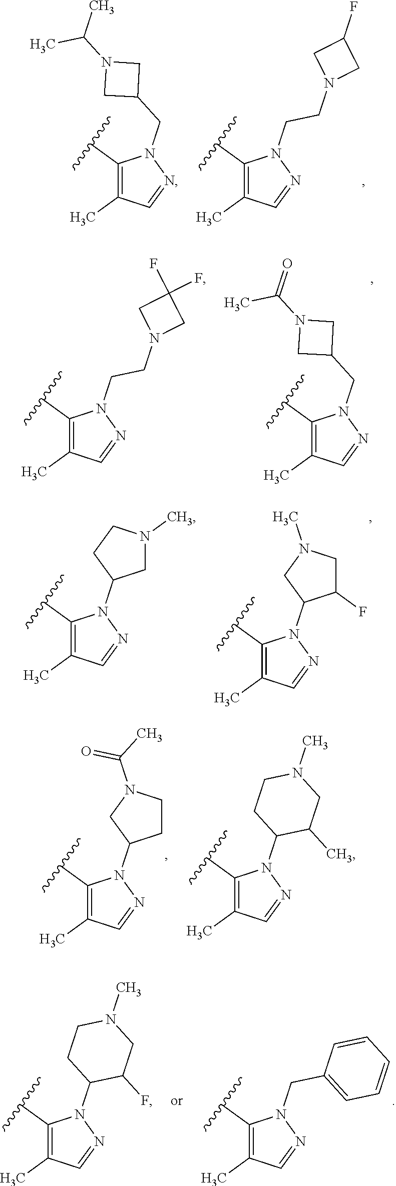

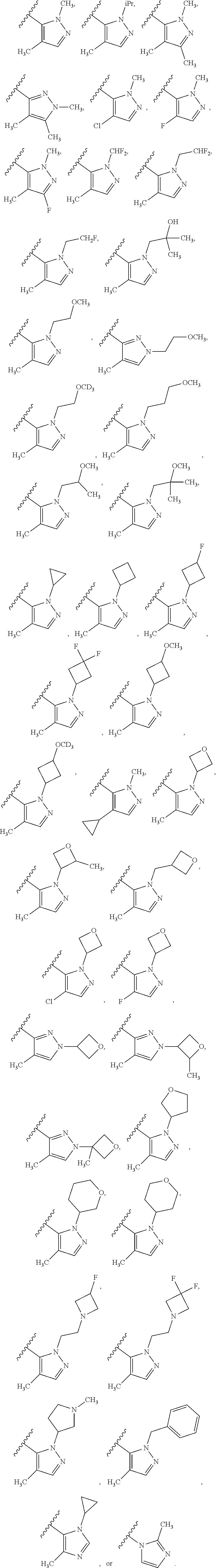

In some cases, Z is
In some cases, Z is

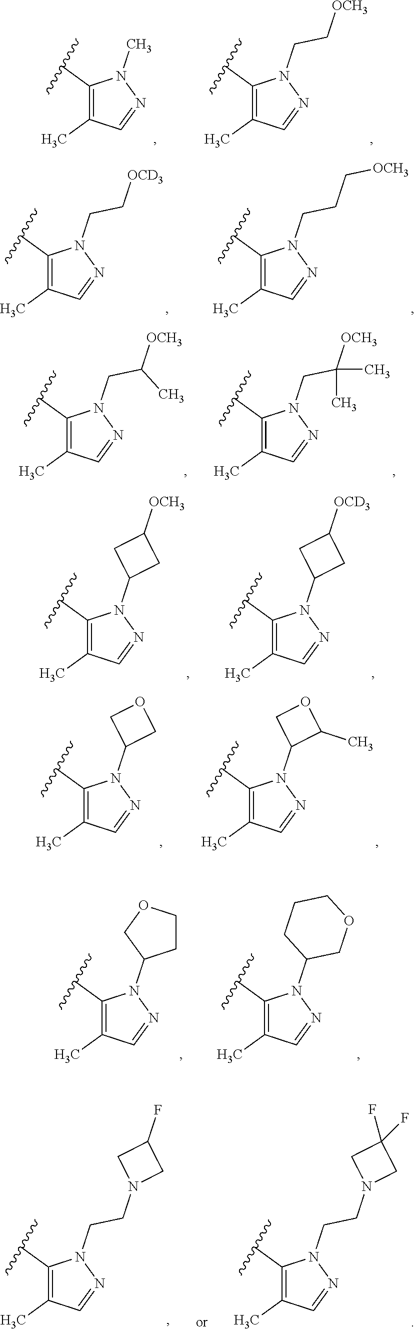

In some cases, Z is

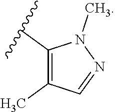 [SMILES: *c1c(C)cnn1C]

In some cases, Z is

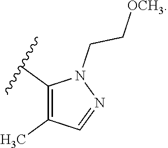 [SMILES: *c1c(C)cnn1CCOC]

In some cases, Z is

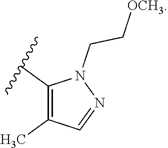 [SMILES: *c1c(C)cnn1CCOC]

In some cases, Z is

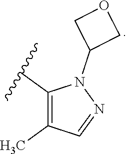 [SMILES: *c1c(C)cnn1C1COC1]

In some cases, Z is

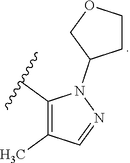 [SMILES: *c1c(C)cnn1C1CCOC1]

In some cases, Z is

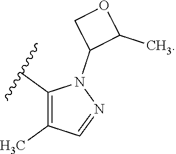 [SMILES: *c1c(C)cnn1C1COC1C]

In some cases, Z is

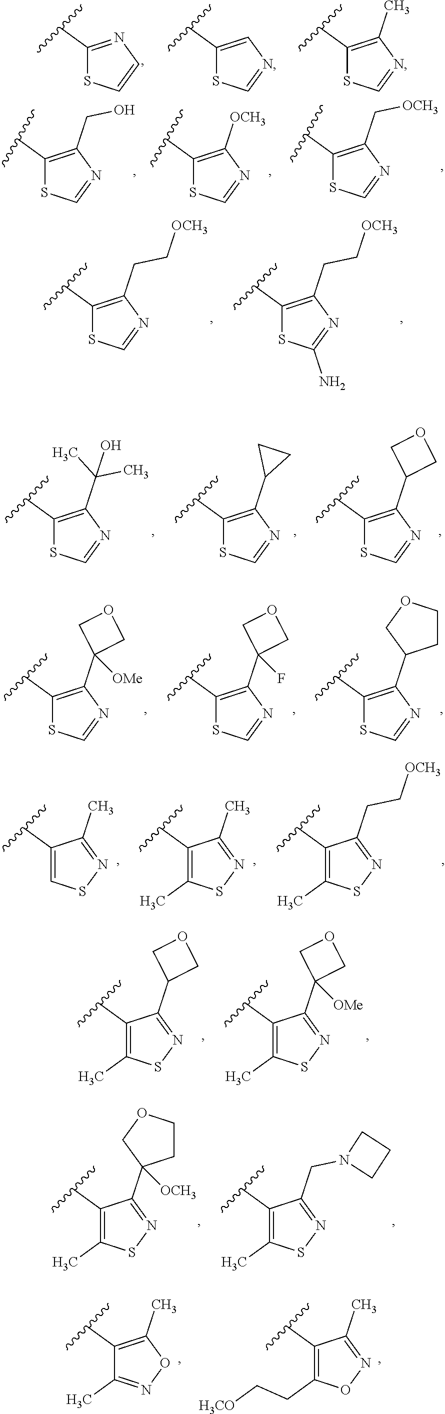

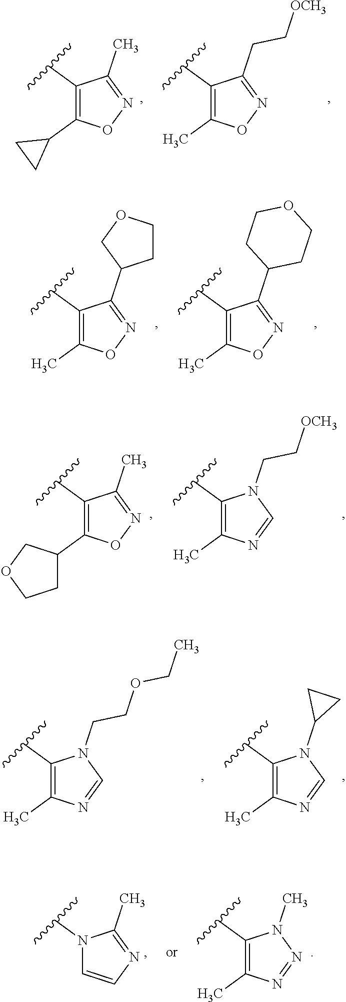

In some cases, Z is

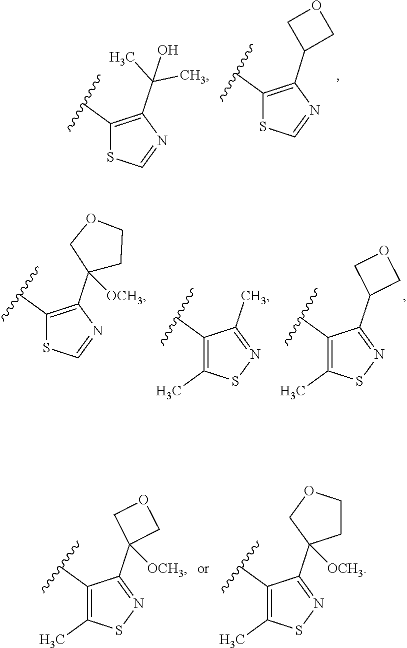

In some cases, Z is

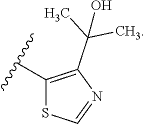 [SMILES: *c1scnc1C(C)(C)O]

In some cases, Z is

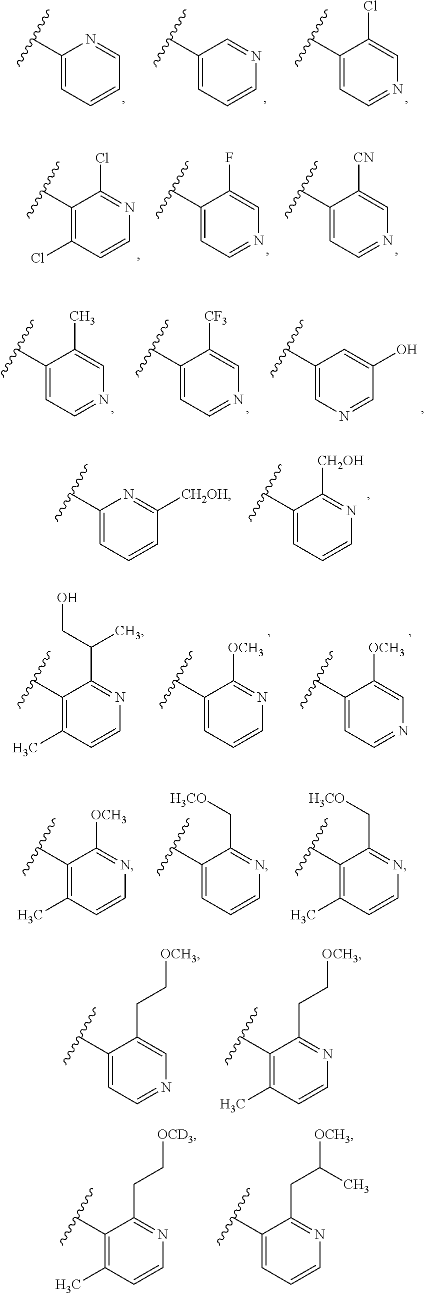

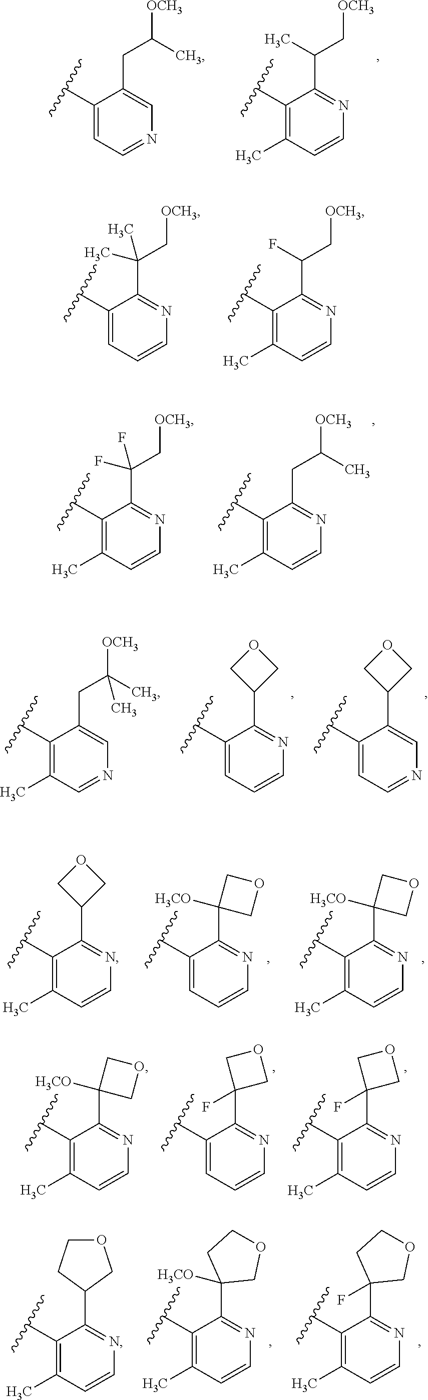

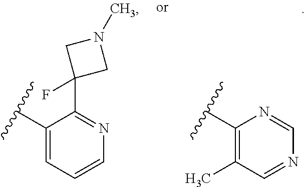

In some cases, Z is

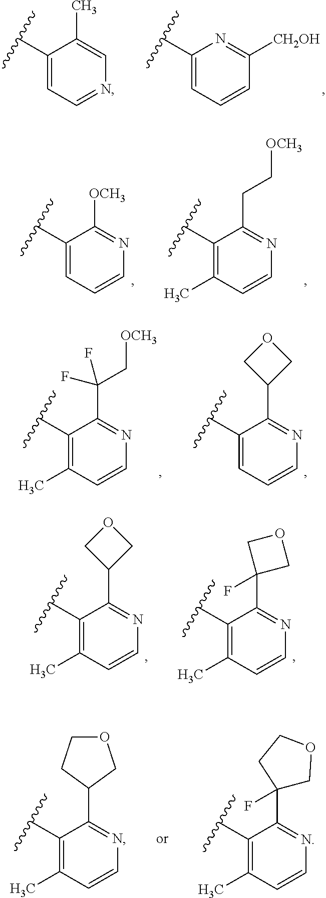

In some cases, Z is

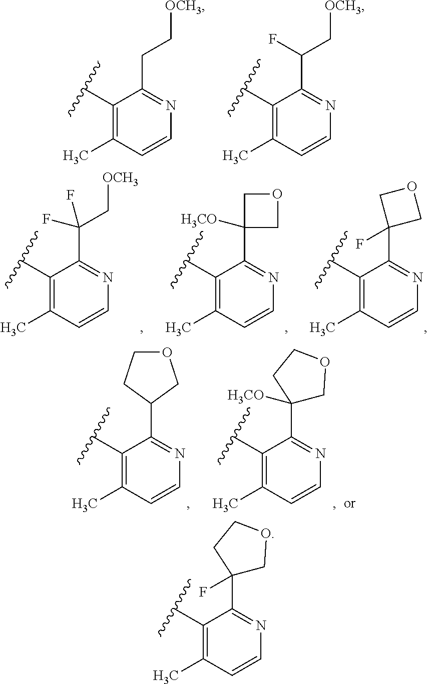

In some cases, Z is a bicyclic ring comprising a heteroaryl ring having 5 or 6 total ring atoms and 1-3 heteroatoms selected from N, O, and S fused to a cycloalkyl ring having 5 or 6 total ring atoms or a heterocycloalkyl ring having 5 or 6 total ring atoms and 1 or 2 heteroatoms selected from N, O, and S; wherein the bicyclic ring is optionally substituted with 1-4 substituents. In some cases, the heteroaryl ring of the bicyclic ring is pyridyl, pyridazinyl, pyrimidinyl, or pyrazinyl; and the heterocycloalkyl ring of the bicyclic ring is pyrrolidinyl, tetrahydrofuranyl, tetrahydropyranyl, or tetrahydrothiophenyl. In some cases, the heteroaryl group is pyridyl and the heterocycloalkyl group is furanyl. In some cases, the bicyclic ring is unsubstituted. In some cases, the bicyclic ring is substituted with 1-4 substituents selected from halo, CN, C₁₋₆alkyl, C₁₋₃haloalkyl, C₀₋₆alkylene-OH, and C₀₋₆alkylene-C₁₋₃alkoxy. In some cases, Z is

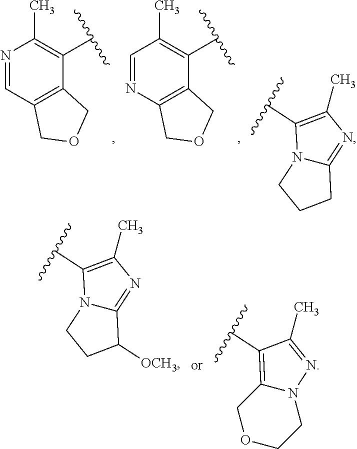

In some cases,

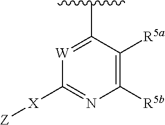

### of Formula (I) is

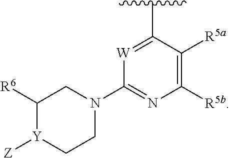

In some cases,

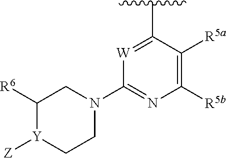

exhibits the following stereochemical configuration:

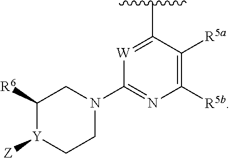

In some cases,

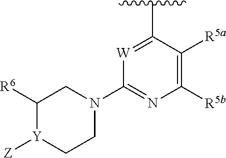

exhibits the following stereochemical configuration:

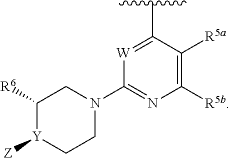

In some cases, the disclosure provides compounds of Formula (I′):

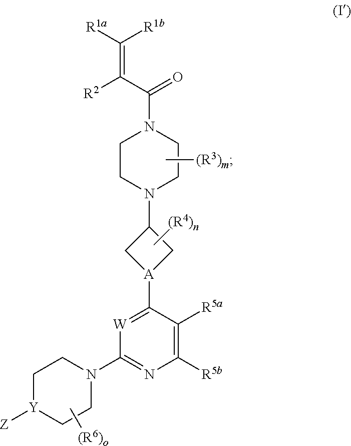

and pharmaceutically acceptable salts thereof, wherein the substituents are as previously described herein.

In some cases, A is CH, C-halo, C—CN, C—C₁₋₃alkyl, C—C₁₋₃haloalkyl, C—C₀₋₃alkyleneOH, or C—C₀₋₃alkylene-C₁₋₃alkoxy; and X is

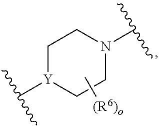

and the disclosure provides compounds of Formula (IA):

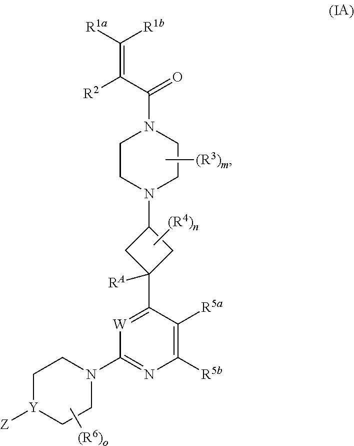

and pharmaceutically acceptable salts thereof, wherein R⁴ is H, halo, CN, C₁₋₃alkyl, C₁₋₃haloalkyl, C₀₋₃alkyleneOH, or C₀₋₃alkylene-C₁₋₄alkoxy; and the remaining substituents are as previously defined herein.

In some cases, A is N and X is

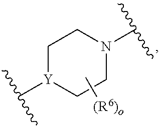

and the disclosure provides compounds of Formula (IB):

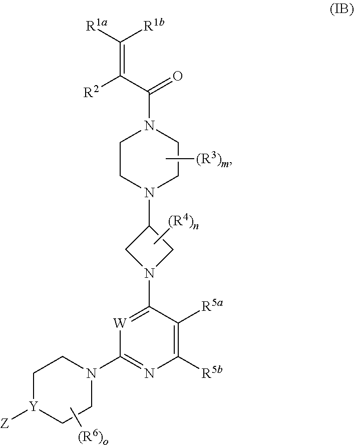

and pharmaceutically acceptable salts thereof, wherein the substituents are as previously defined herein.

In some cases, A is N, X is

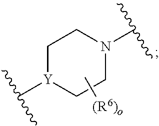

and R^{5a} and an R⁴, together with the atoms to which they are attached, form an optionally substituted ring having 6-10 total ring atoms and 0, 1, or 2 heteroatoms selected from N, O, and S, wherein the ring is saturated or unsaturated, and the rest of the substituents are as defined herein. Contemplated compounds include, but are not limited to:

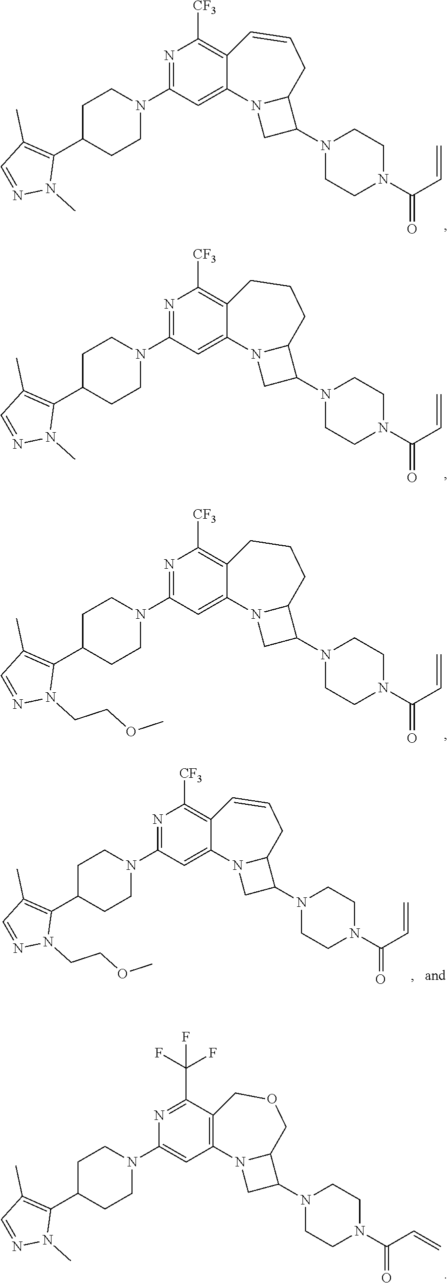 [SMILES: C=CC(=O)N1CCN(C2CN3c4cc(N5CCC(c6c(C)cnn6C)CC5)nc(C(F)(F)F)c4C=CCC23)CC1 ; C=CC(=O)N1CCN(C2CN3c4cc(N5CCC(c6c(C)cnn6C)CC5)nc(C(F)(F)F)c4CCCC23)CC1 ; C=CC(=O)N1CCN(C2CN3c4cc(N5CCC(c6c(C)cnn6CCOC)CC5)nc(C(F)(F)F)c4CCCC23)CC1 ; C=CC(=O)N1CCN(C2CN3c4cc(N5CCC(c6c(C)cnn6CCOC)CC5)nc(C(F)(F)F)c4C=CCC23)CC1 ; C=CC(=O)N1CCN(C2CN3c4cc(N5CCC(c6c(C)cnn6C)CC5)nc(C(F)(F)F)c4COCC23)CC1]

In some cases, X is

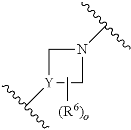

and the disclosure provides compounds of Formula (IE):

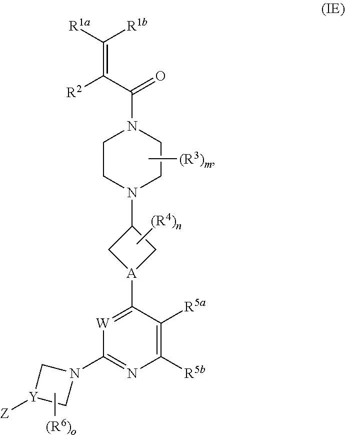

and pharmaceutically acceptable salts thereof.

In some cases, X is

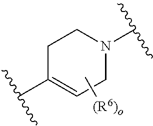

and the disclosure provides compounds of Formula (IF):

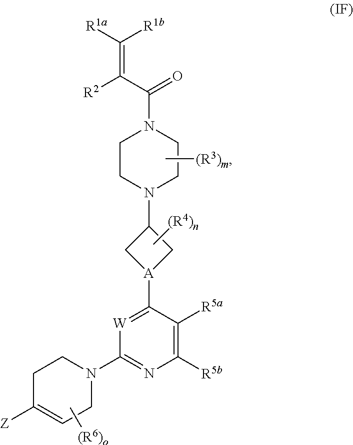

and pharmaceutically acceptable salts thereof.

In some cases, X is

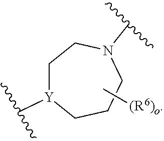

and the disclosure provides compounds of Formula (IG):

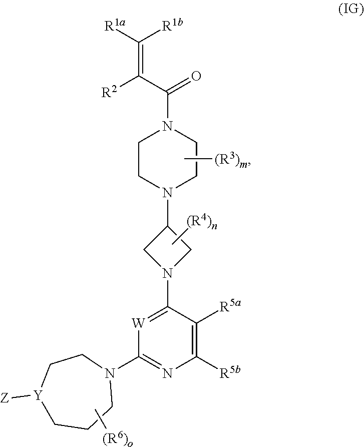

and pharmaceutically acceptable salts thereof.

In some cases, the disclosure provides compounds of Formula (I) wherein A is N; X is

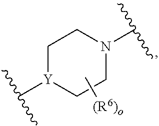

and Z is optionally substituted phenyl or pyridyl.

In some cases, the disclosure provides a compound listed in Table E, below. If the stereochemistry of a structure or a portion of a structure in Table E is not explicitly shown (e.g., such as with dashed or bold lines), then the structure or portion of structure is either achiral or interpreted as being any of the possible stereoisomers of the structure or portion of the structure. In cases in which the stereochemistry of the structure or portion of the structure in Table E is explicitly shown, a single stereoisomer of the structure or portion of a structure is represented.

TABLE E

| Comp. # | Structure | Name |
|---|---|---|
| 1-098 | 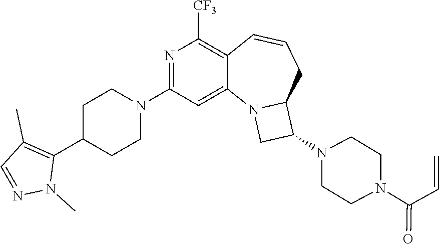 [SMILES: C=CC(=O)N1CCN([C@@H]2CN3c4cc(N5CCC(c6c(C)cnn6C)CC5)nc(C(F)(F)F)c4C=CC[C@@H]23)CC1] | 1-(4-((7aS,8R)-2-(4-(1,4-Dimethyl- 1H-pyrazol-5-yl)piperidin-1-yl)-4- (trifluoromethyl)-7,7a,8,9- tetrahydroazeto[1,2-a]pyrido[3,4- f]azepin-8-yl)piperazin-1-yl)prop-2- en-1-one |
| 1-099 | 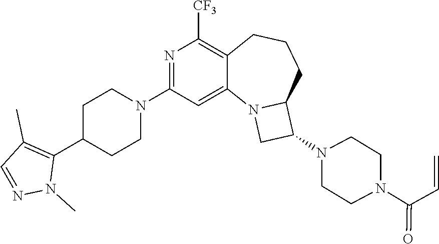 [SMILES: C=CC(=O)N1CCN([C@@H]2CN3c4cc(N5CCC(c6c(C)cnn6C)CC5)nc(C(F)(F)F)c4CCC[C@@H]23)CC1] | 1-(4-((7aS,8R)-2-(4-(1,4-Dimethyl- 1H-pyrazol-5-yl)piperidin-1-yl)-4- (trifluoromethyl)-5,6,7,7a,8,9- hexahydroazeto[1,2-a]pyrido[3,4- f]azepin-8-yl)piperazin-1-yl)prop-2- en-1-one |
| 1-099-1 | 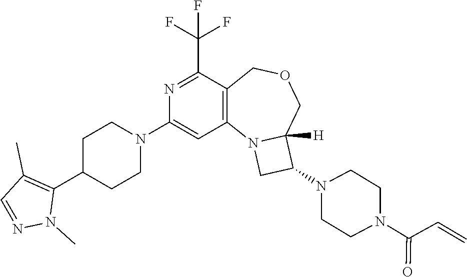 [SMILES: C=CC(=O)N1CCN([C@@H]2CN3c4cc(N5CCC(c6c(C)cnn6C)CC5)nc(C(F)(F)F)c4COC[C@H]23)CC1] | 1-(4-((7aR,8R)-2-(4-(1,4-dimethyl- 1H-pyrazol-5-yl)-1-piperidinyl)-4- (trifluoromethyl)-5,6,7,7a,8,9- hexahydroazeto[1,2-a]pyrido[3,4- f]azepin-8-yl)-1-piperazinyl)-2- propen-1-one |
| 1-100 | 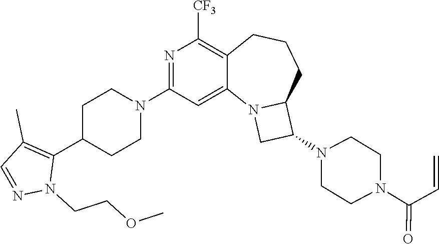 [SMILES: C=CC(=O)N1CCN([C@@H]2CN3c4cc(N5CCC(c6c(C)cnn6CCOC)CC5)nc(C(F)(F)F)c4CCC[C@@H]23)CC1] | 1-(4-((7aS,8R)-2-(4-(1-(2- Methoxyethyl)-4-methyl-1H-pyrazol- 5-yl)piperidin-1-yl)-4- (trifluoromethyl)-5,6,7,7a,8,9- hexahydroazeto[1,2-a]pyrido[3,4- f]azepin-8-yl)piperazin-1-yl)prop-2- en-1-one |
| 1-097 | 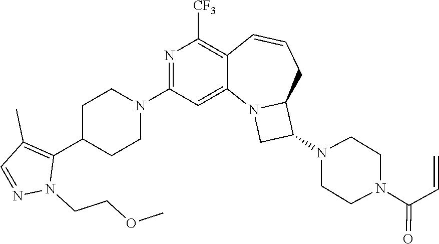 [SMILES: C=CC(=O)N1CCN([C@@H]2CN3c4cc(N5CCC(c6c(C)cnn6CCOC)CC5)nc(C(F)(F)F)c4C=CC[C@@H]23)CC1] | 1-(4-((7aS,8R)-2-(4-(1-(2- Methoxyethyl)-4-methyl-1H-pyrazol- 5-yl)piperidin-1-yl)-4- (trifluoromethyl)-7,7a,8,9- tetrahydroazeto[1,2-a]pyrido[3,4- f]azepin-8-yl)piperazin-1-yl)prop-2- en-1-one |
| 1-104 | 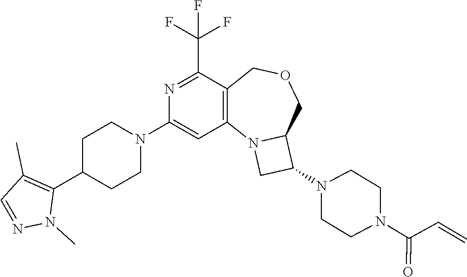 [SMILES: C=CC(=O)N1CCN([C@@H]2CN3c4cc(N5CCC(c6c(C)cnn6C)CC5)nc(C(F)(F)F)c4COC[C@@H]23)CC1] | 1-(4-((7aR,8R)-2-(4-(1,4,-dimethyl- 1H-pyrazol-5-yl)-1-piperidinyl)-4- (trifluoromethyl)-7,7a,8,9-tetrahydro- 5H-azeto[2,1-c]pyrido[4,3- e][1,4]oxazepin-8-yl)-1-piperazinyl)- 2-propen-1-one |

In some cases, A is N and X is

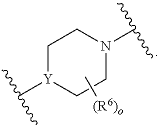

In some cases, the compound of Formula (I) or Formula (IB) is selected from compound 1-119 to 1-123, and 1-152, or a pharmaceutically acceptable salt of any of the foregoing. In some cases, A is N, X is

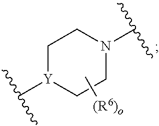

and R^{5a} and an R⁴, together with the atoms to which they are attached, form an optionally substituted ring having 6-10 total ring atoms and 0, 1, or 2 heteroatoms selected from N, O, and S, wherein the ring is saturated or unsaturated. In some cases, the compound of Formula (I) or Formula (IB) is selected from compound 1-119 to 1-123 and 1-152, or a pharmaceutically acceptable salt of any of the foregoing.

### Example of Formula (I)

For example, provided herein are compounds of Formula (I),

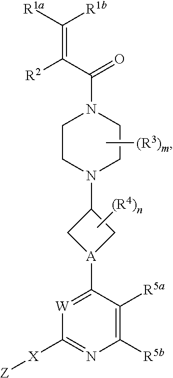

or pharmaceutically acceptable salts thereof, wherein the substituents are as defined in this “Example of Formula (I)” section.

In some cases,

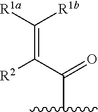

is

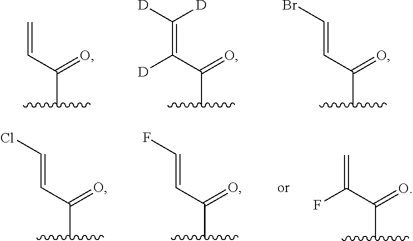

In some cases,

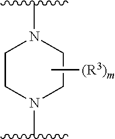

is

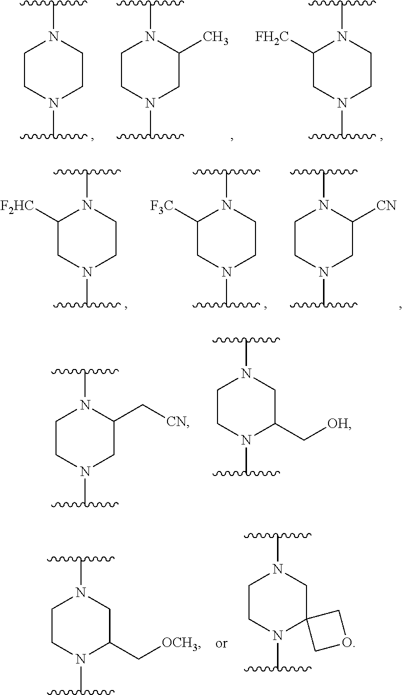

In some cases, A is N.

In some cases, n is 1. In some cases, n is 2. In some cases, the other R⁴ is CH₃, CH₂CH₃, CH₂CH₂CH₃, CH(CH₃)₂, CF₃, CHF₂, or CH₂F. In some cases, W is CH

In some cases, one R⁴ and R^{5a}, together with the atoms to which they are attached, form an optionally substituted ring that is saturated. In some cases, one R⁴ and R^{5a}, together with the atoms to which they are attached, form an optionally substituted ring that is unsaturated. In some cases, one R⁴ and R^{5a}, together with the atoms to which they are attached, form an optionally substituted ring that has 6 total ring atoms. In some cases, one R⁴ and R^{5a}, together with the atoms to which they are attached, form an optionally substituted ring that has 7 total ring atoms. In some cases, one R⁴ and R^{5a}, together with the atoms to which they are attached, form an optionally substituted ring that has 8 total ring atoms. In some cases, one R⁴ and R^{5a}, together with the atoms to which they are attached, form an optionally substituted ring that has 9 or 10 total ring atoms. In some cases, one R⁴ and R^{5a}, together with the atoms to which they are attached, form an optionally substituted ring that has 0 heteroatoms. In some cases, one R⁴ and R^{5a}, together with the atoms to which they are attached, form an optionally substituted ring that has 1 or 2 heteroatoms selected from N, O, and S. In some cases, the 1 or 2 heteroatoms are each O. In some cases, one R⁴ and R^{5a}, together with the atoms to which they are attached, form an optionally substituted ring that is an ether. In some cases, the 1 or 2 heteroatoms are each N. In some cases, one R⁴ and R^{3a}, together with the atoms to which they are attached, form an optionally substituted ring that is a lactam or a cyclic amine. In some cases, one R⁴ and R^{5a}, together with the atoms to which they are attached, form a ring that is unsubstituted. In some cases, one R⁴ and R^{5a}, together with the atoms to which they are attached, form a ring that is substituted with 1 or 2 substituents selected from the group consisting of C₁₋₃alkyl, C₁₋₃haloalkyl, oxo, halo, CN, C₀₋₃alkyleneOH, C₀₋₂alkylene-C₁₋₃alkoxy, cycloalkyl having 3-7 total ring atoms, cycloalkenyl having 5-7 total ring atoms, heterocycloalkyl having 4-7 total ring atoms and 1-3 heteroatoms selected from N, O, and S, heterocycloalkenyl having 5-7 total ring atoms and 1-3 heteroatoms selected from N, O, and S, and phenyl. In some cases,

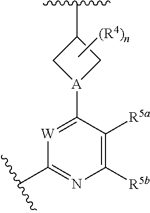

is

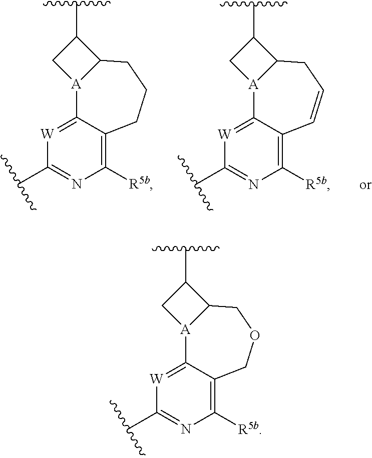

In some cases, R^{5b} is CF₃, CF₂H, CFH₂, or CF₂CH₃. In some cases, R⁵ is CF₃. In some cases, R^{5b} is CF₂H. In some cases, R^{5b} is CFH₂. In some cases, R^{5b} is CF₂CH₃.

In some cases, X is

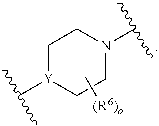

In some cases, Y is C—H. In some cases, o is 0. In some cases, o is 1. In some cases, R⁶ is CH₃, CH₂F, CHF₂, or CF₃. In some cases,

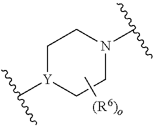

is

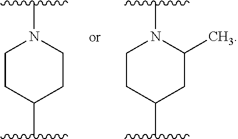 [SMILES: *C1CCN(*)CC1 ; *C1CCN(*)C(C)C1]

In some cases,

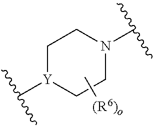

is

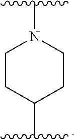 [SMILES: *C1CCN(*)CC1]

In some cases

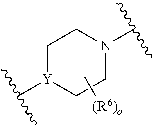

is

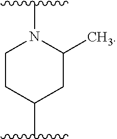 [SMILES: *C1CCN(*)C(C)C1]

In some cases, Z is heteroaryl comprising 5 or 6 total ring atoms and 1-3 heteroatoms selected from N, O, and S, wherein the heteroaryl is optionally substituted with 1-4 substituents. In some cases, the heteroaryl is pyrrolyl, furanyl, thiophenyl, pyrazolyl, imidazolyl, thiazolyl, isothiazolyl, oxazolyl, isoxazolyl, triazolyl, thiadiazolyl, oxadiazolyl, pyridyl, pyridazinyl, pyrimidinyl, pyrazinyl, or triazinyl. In some cases, the heteroaryl is pyrazolyl or pyridyl. In some cases, the heteroaryl is substituted with 1-4 substituents, each of which independently is selected from the group consisting of halo, CN, C₁₋₆alkyl, C₁₋₆haloalkyl, C₂₋₆alkenyl, C₂₋₆ haloalkenyl, C₀₋₆alkylene-OH, C₀₋₃alkylene-C₁₋₃alkoxy, C₀₋₆alkylene-N(R^{N1})₂ wherein each R^{N1} independently is H or C₁₋₃alkyl. C₀₋₂alkylene-cycloalkyl having 3-6 total ring atoms, C₀₋₂alkylene-heterocycloalkyl having 3-6 total ring atoms and 1-3 heteroatoms selected from N, O, and S, and C₀₋₂alkylene-phenyl; wherein each of the alkyl, alkenyl, C₀₋₃alkylene-C₁₋₃alkoxy, cycloalkyl, heterocycloalkyl, and phenyl substituents independently is optionally substituted with 1-3 substituents independently selected from deuterium, halo, OH, CH₃, OCH₃, and OCD₃, In some cases, each of the 1-4 substituents independently is selected from the group consisting of Cl, F, CN, CH₃, CD₃, CH₂CH₃, CH(CH₃)₂, CF₃, CHF₂, CH₂F, CH₂CHF₂, CH₂CH₂F, CH(CH₂F)₂, CH(CH₃)CH₂F, CH(CH₃)CHF₂, C(═CH₂)CH₂F, OH, CH₂OH, CH₂CH₂OH, CH(CH)CH₂OH. C(CH₃)₂OH, C(CH₃)₂CH₂OH, CH₂C(CH₃)₂OH, OCH₃, OCD₃, CH₂OCH₃, CH₂OCD₃, CH—CH₂OCH₃, CHFCH₂OCH₃, CF₂CH₂OCH₃, CH₂CH₂OCD₃, CH₂CH₂OCH₂CH₃,CH₂CH₂CH₂OCH₃, CH₂CH₂CH₂OCD₃, CH(CH₃)CH₂OCH₃, CH(CH₃)CH₂OCD₃,C(CH₃)₂CH₂OCH₃, C(CH₃)₂CH₂OCD₃, CH₂CH(CH₃)OCH₃, CH₂CH(CH₃)OCD₃, CH₂C(CH₃)OCH₃ CH₂C(CH₃): OCD₃, NH₂, CH₂NH₂, CH₂NHCH₃, CH₂N(CH₃)₂, CH—CH₂NH₂, CH₂CH₂NHCH₃, CH₂CH₂N(CH₃)₂,

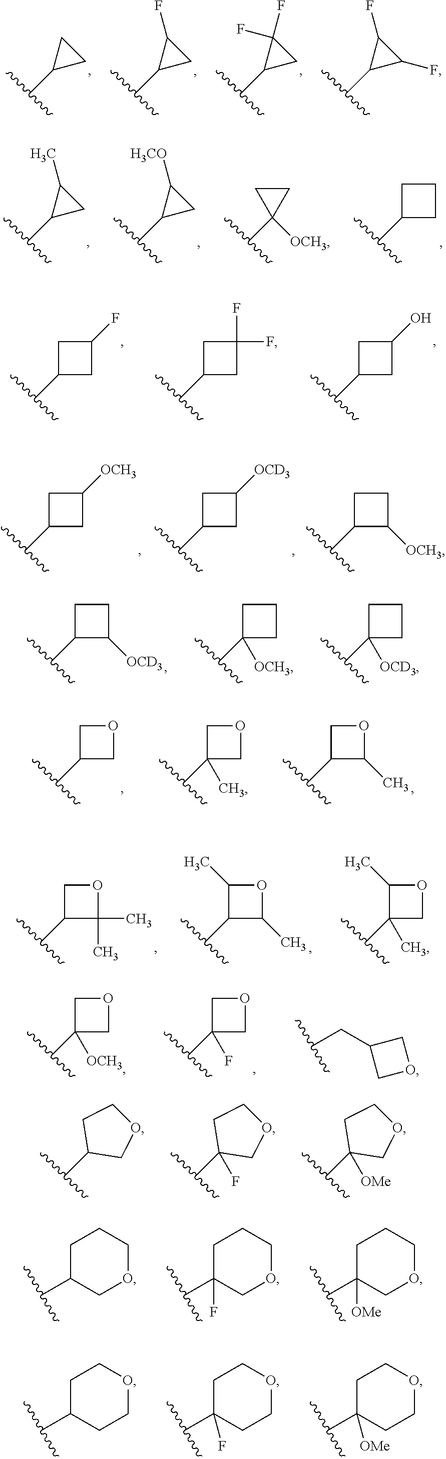

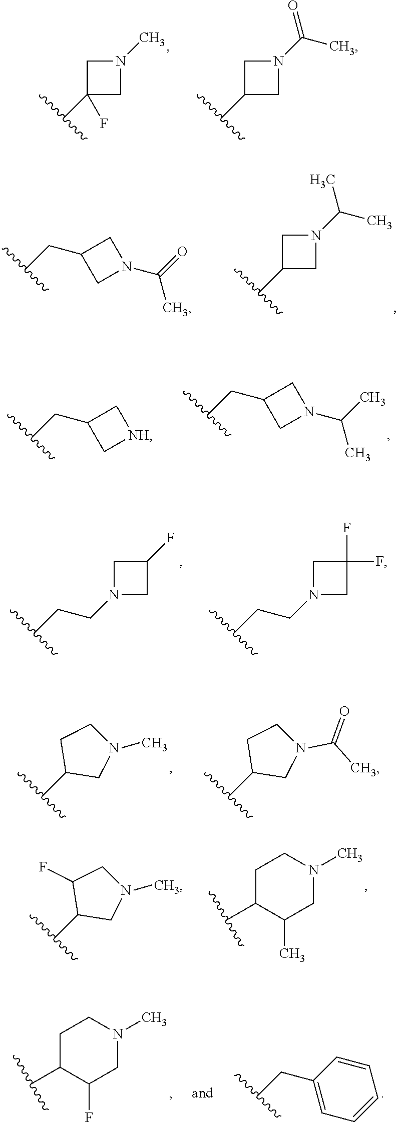

In some cases, each of the 1-4 substituents independently is CH₃, CH₂CH₂OCH₃, CH₂CH₂OCD₃,

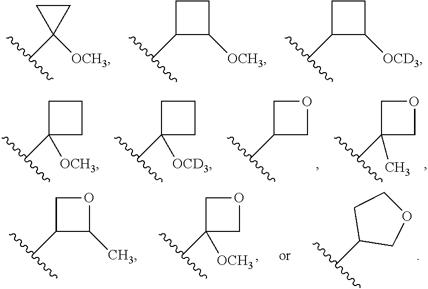

In some cases, Z is

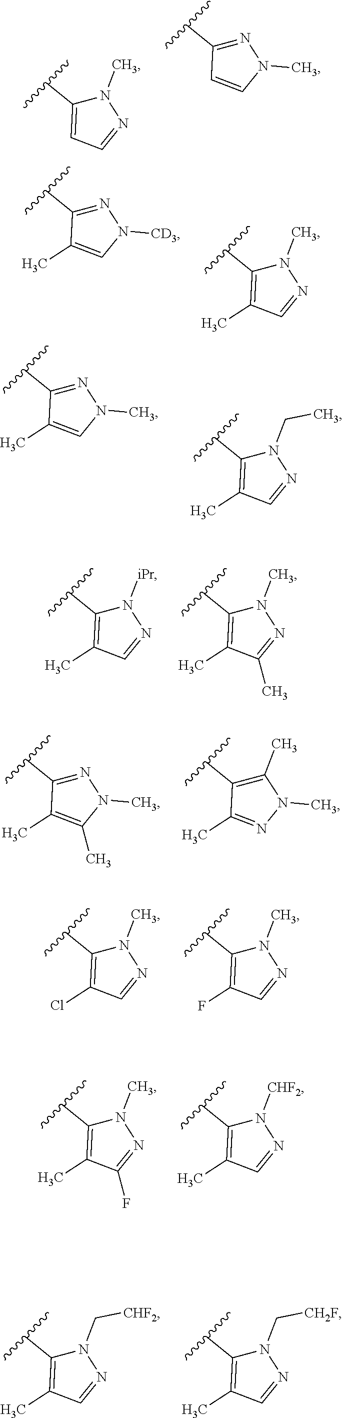

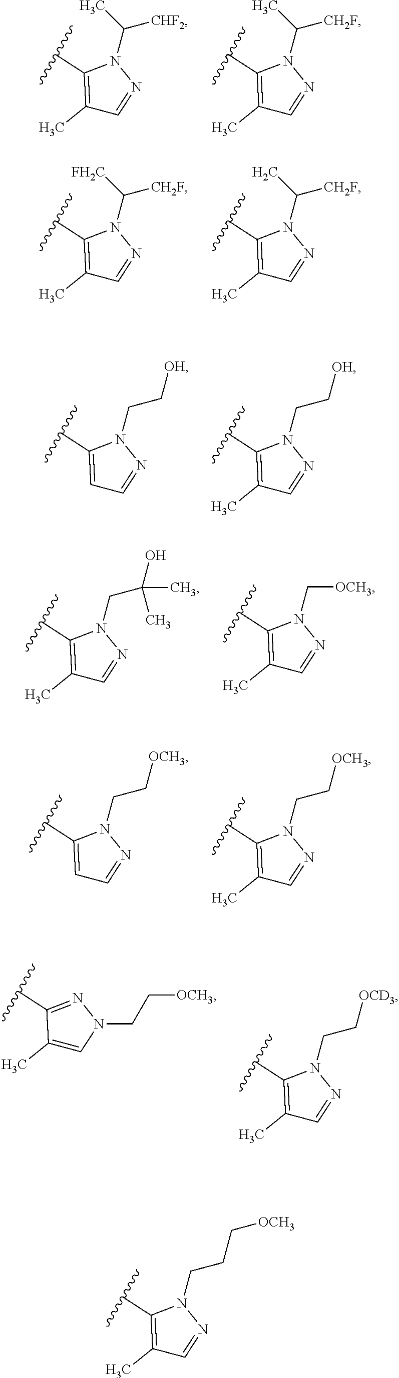

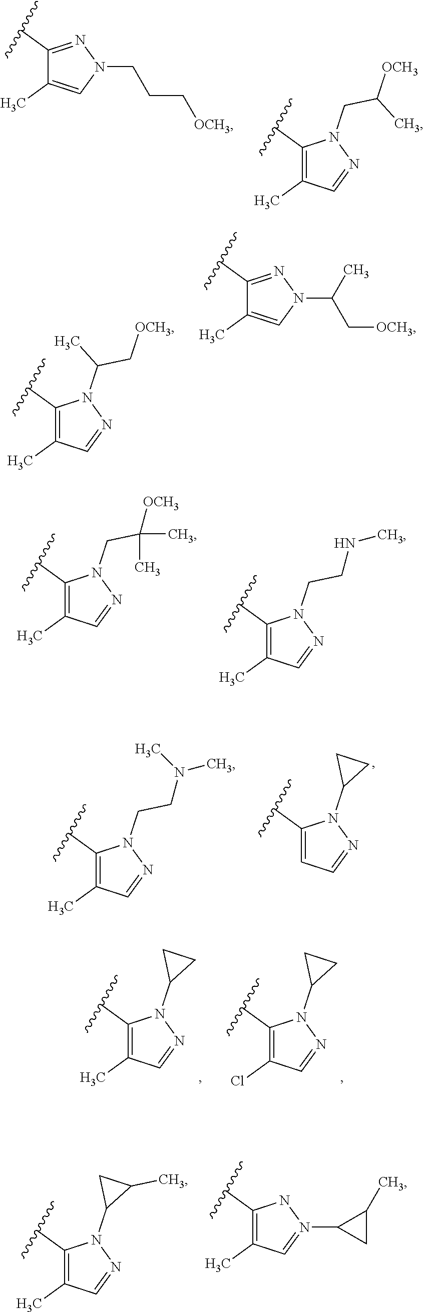

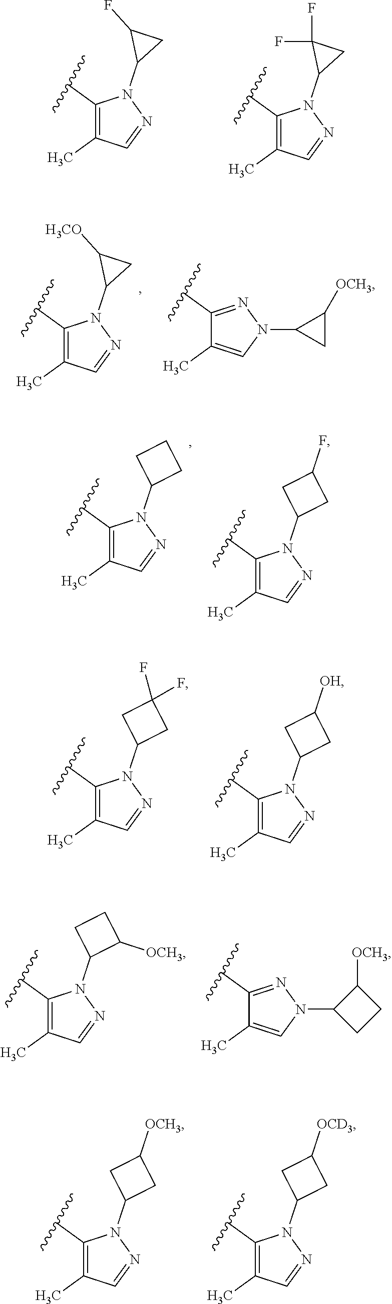

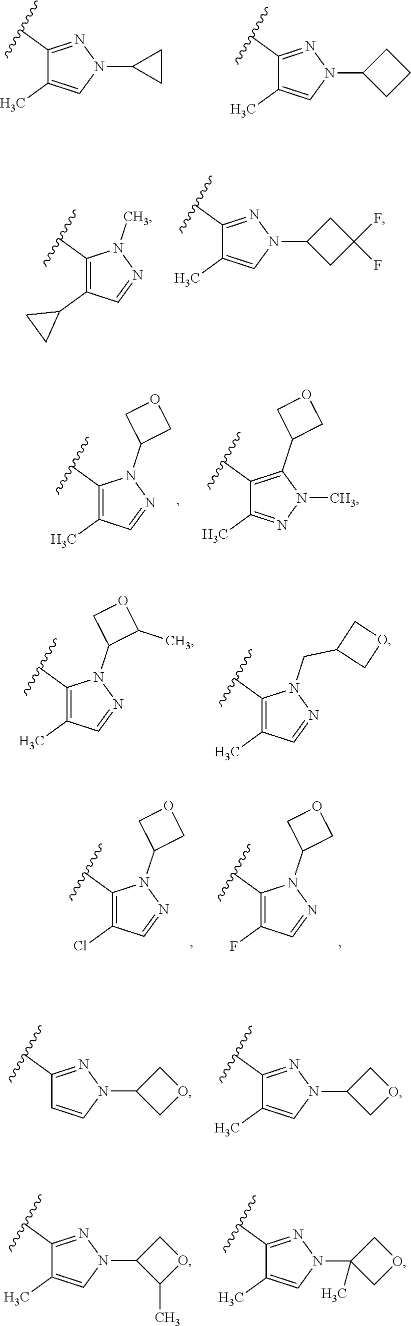

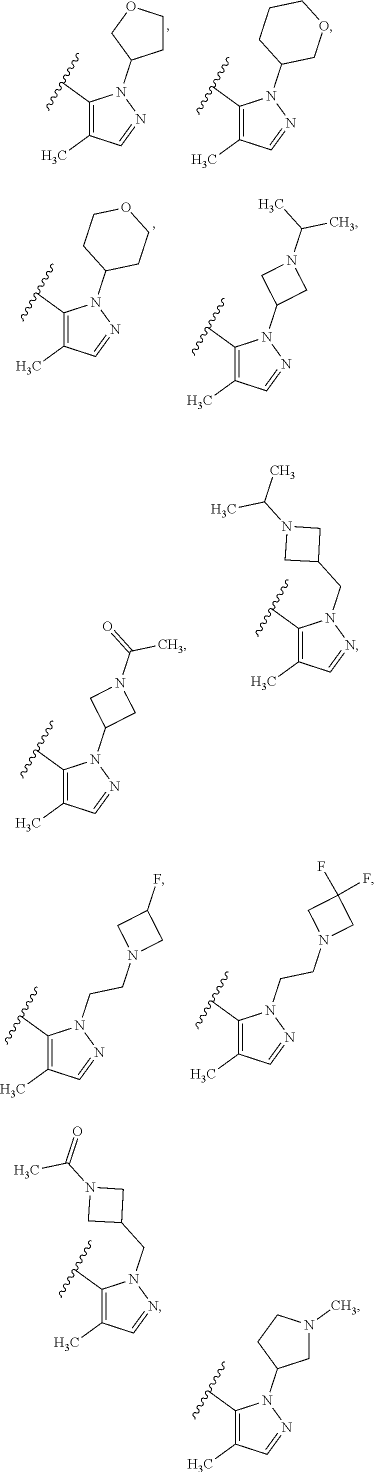

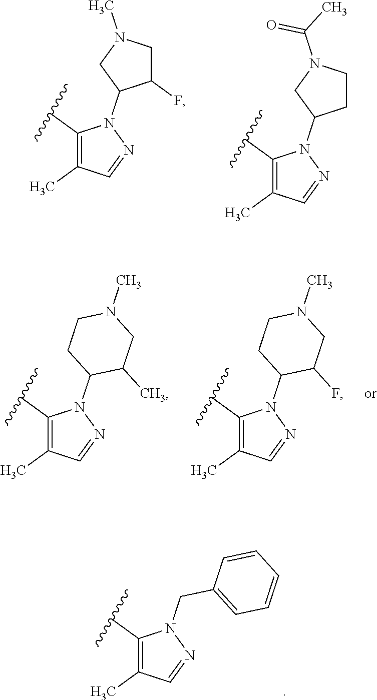 [SMILES: *c1c(C)cnn1C1CN(C)CC1F ; *c1c(C)cnn1C1CCN(C(C)=O)C1 ; *c1c(C)cnn1C1CCN(C)CC1C ; *c1c(C)cnn1C1CCN(C)CC1F ; *c1c(C)cnn1Cc1ccccc1]

In some cases, Z is

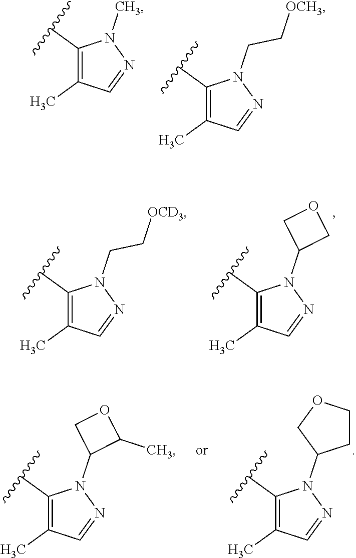

In some cases, Z is

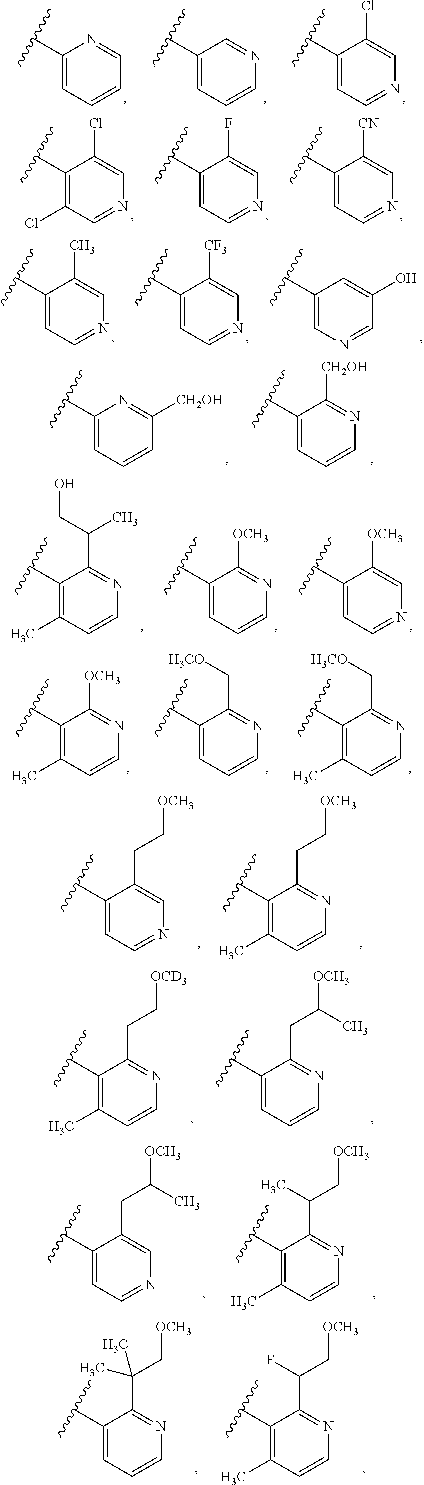

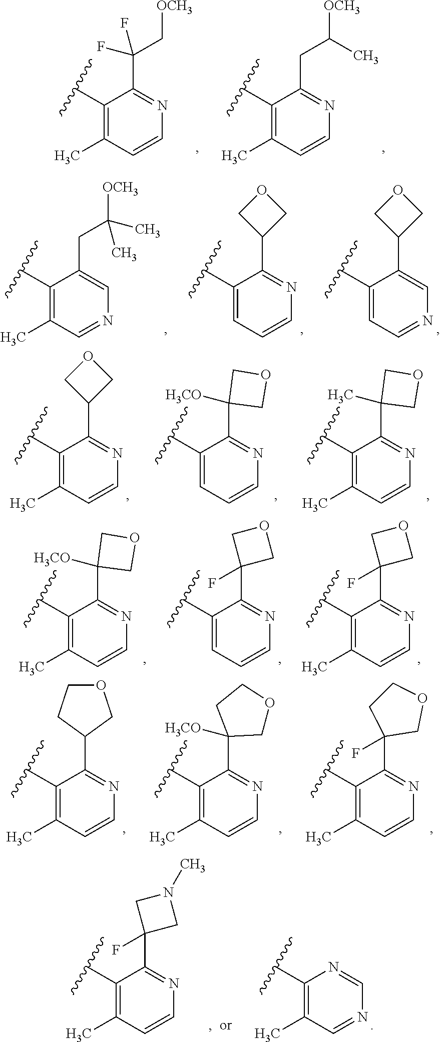

In some cases, Z is

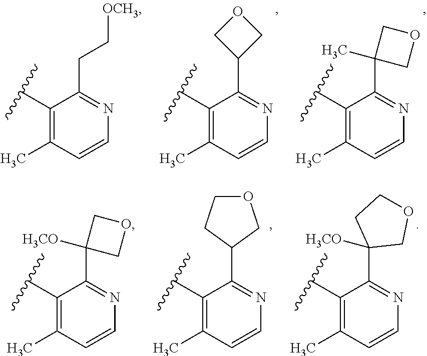

In some cases, Z is

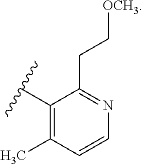 [SMILES: *c1c(C)ccnc1CCOC]

In some cases, Z is

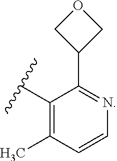 [SMILES: *c1c(C)ccnc1C1COC1]

In some cases, Z is

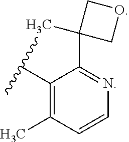 [SMILES: *c1c(C)ccnc1C1(C)COC1]

In some cases, Z is

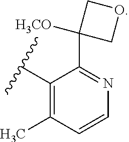 [SMILES: *c1c(C)ccnc1C1(OC)COC1]

In some cases, Z is

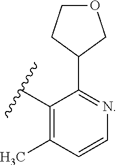 [SMILES: *c1c(C)ccnc1C1CCOC1]

In some cases, Z is

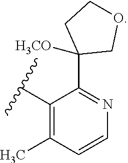 [SMILES: *c1c(C)ccnc1C1(OC)CCOC1]

Further provided herein is a compound of Formula (I), or a pharmaceutically acceptable salt thereof, wherein

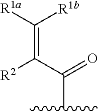

is

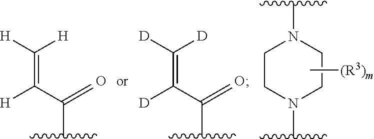

is

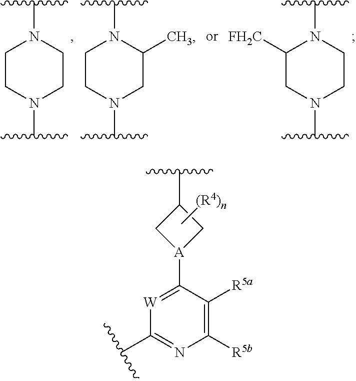

is

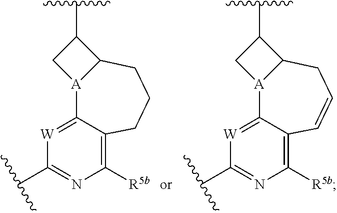

### X is

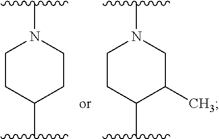

and Z is pyrazolyl or pyridyl, each of which is optionally substituted with 1-4 substituents. In some cases, each of the 1-4 substituents of Z independently is CH₃, CHCH₂OCH₃, CH₂CH₂OCD₃.

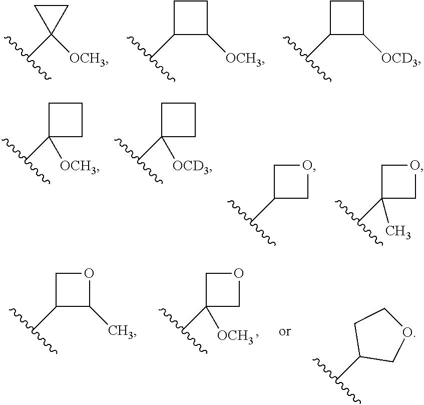

In some cases, Z is substituted with 2 substituents. In some cases, at least one substituent is CH₃. In some cases, each substituent is CH₃. In some cases, Z is substituted with CH, and CH₂CH₂OCH₃. In some cases, Z is substituted with CH₃ and

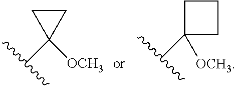 [SMILES: *C1(OC)CC1 ; *C1(OC)CCC1]

In some cases, Z is substituted with CH₃ and

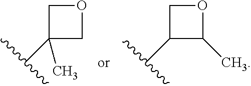

In some cases, Z is substituted with CH₃ and

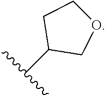 [SMILES: *C1CCOC1]

In some cases, Z is

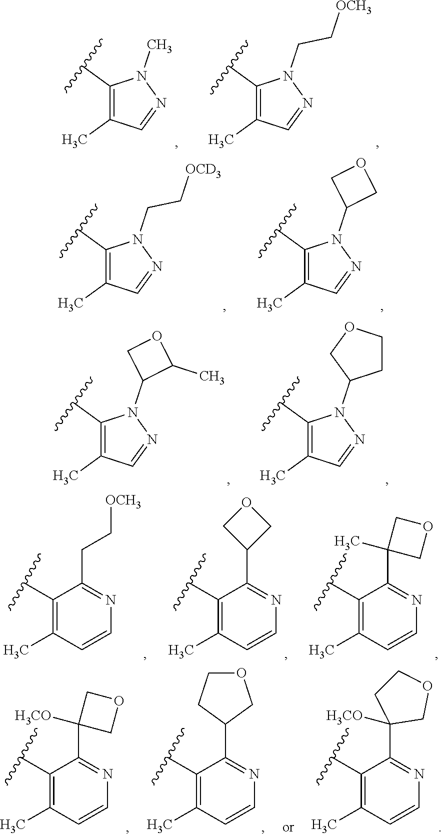

In some cases, Z is

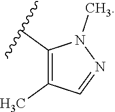 [SMILES: *c1c(C)cnn1C]

In some cases, Z is

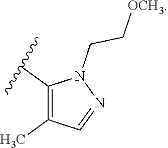 [SMILES: *c1c(C)cnn1CCOC]

In some cases,

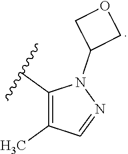 [SMILES: *c1c(C)cnn1C1COC1]

In some cases, Z is

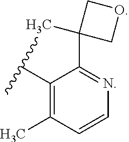 [SMILES: *c1c(C)ccnc1C1(C)COC1]

In some cases, Z is

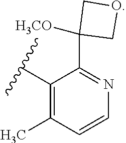 [SMILES: *c1c(C)ccnc1C1(OC)COC1]

It is understood that selections of values of each variable are those that result in the formation of stable or chemically feasible compounds.

### Biological Activity

In some cases, the compounds disclosed herein (e.g., compounds of Formula (I), Formula (I′). Formula (IA), Formula (IB), Formula (IE), Formula (IF), Formula (IG), Formula (II), Formula (II′), Formula (IIA), Formula (IIB), Formula (IIC), Formula (IID), Formula (IIE), and Formula (IIF), and compounds listed in Table A, Table A′, Table B, Table B′, and Table E), and pharmaceutically acceptable salts of the foregoing, have an IC₅₀ value of less than 5 μM, or less than 4 μM, or less than 3 μM, or less than 2 μM, or less than 1 μM, or less than 0.9 μM, or less than 0.7 μM, or less than 0.6 μM, or less than 0.5 μM, or less than 0.4 μM, or less than 0.3 μM, or less than 0.2 μM, or less than 0.1 μM, or less than 0.09 μM, or less than 0.08 UM, or less than 0.07 μM, or less than 0.06 μM, or less than 0.05 μM, or less than 0.04 μM, or less than 0.03 μM, or less than 0.02 μM, or less than 0.01 μM in the coupled exchange assay, which is described in the “BIOLOGICAL EVALUATION” section. In some cases, the compounds disclosed herein, and pharmaceutically acceptable salts of the foregoing, have an IC₅₀ value of less than 1 μM. In some cases, the compounds disclosed herein, and pharmaceutically acceptable salts of the foregoing, have an IC₅₀ value of less than 0.5 μM. In some cases, the compounds disclosed herein, and pharmaceutically acceptable salts of the foregoing, have an IC₅₀ value of less than 0.3 μM. In some cases, the compounds disclosed herein, and pharmaceutically acceptable salts of the foregoing, have an IC₅₀ value of less than 0.1 μM. Also provided herein are compounds of the disclosure, and pharmaceutically acceptable salts of the foregoing, having an IC₅₀ of less than 5 μM in the 2 h coupled exchange assay described herein. Further provided herein are compounds of the disclosure, and pharmaceutically acceptable salts of the foregoing, having an IC₅₀ of less than 3 μM in the 2 h coupled exchange assay described herein. Still further provided herein are compounds of the disclosure, or pharmaceutically acceptable salts of the foregoing, having an IC₅₀ of less than 1 μM in the 2 h coupled exchange assay described herein. Still further provided herein are compounds of the disclosure, or pharmaceutically acceptable salts of the foregoing, having an IC₅₀ of less than 0.5 μM in the 2 h coupled exchange assay described herein. Also provided herein are compounds of the disclosure, or pharmaceutically acceptable salts of the foregoing, having an IC₅₀ of less than 0.1 μM in the 2 h coupled exchange assay described herein. Also provided herein are compounds of the disclosure, or pharmaceutically acceptable salts of the foregoing, having an IC₅₀ of less than 0.05 μM in the 2 h coupled exchange assay described herein. Also provided herein are compounds of the disclosure, or pharmaceutically acceptable salts of the foregoing, having an IC₅₀ of less than 0.04 μM in the 2 h coupled exchange assay described herein. Also provided herein are compounds of the disclosure, or pharmaceutically acceptable salts of the foregoing, having an IC₅₀ of less than 0.03 μM in the 2 h coupled exchange assay described herein. Also provided herein are compounds of the disclosure, or pharmaceutically acceptable salts of the foregoing, having an IC₅₀ of less than 0.02 μM in the 2 h coupled exchange assay described herein. Also provided herein are compounds of the disclosure, or pharmaceutically acceptable salts of the foregoing, having an IC₅₀ of less than 0.01 μM in the 2 h coupled exchange assay described herein.

The foregoing merely summarizes certain aspect of this disclosure and is not intended, nor should it be construed, as limiting the disclosure in any way.

### Formulation and Route of Administration

While it may be possible to administer a compound disclosed herein alone in the uses described, the compound administered normally will be present as an active ingredient in a pharmaceutical composition. Thus, further provided herein is a pharmaceutical composition comprising a compound disclosed herein (e.g., compounds of Formula (I), Formula (I′), Formula (IA), Formula (IB), Formula (IE), Formula (IF), Formula (IG), Formula (II), Formula (II′), Formula (IIA), Formula (IIB), Formula (IIC), Formula (IID), Formula (IIE), and Formula (IIF), and compounds listed in Table A, Table A′, Table B, Table B′, and Table E), and pharmaceutically acceptable salts of the foregoing, in combination with one or more pharmaceutically acceptable excipients and, if desired, other active ingredients. See, e.g., Remington: The Science and Practice of Pharmacy, Volume I and Volume II, twenty-second edition, edited by Loyd V. Allen Jr., Philadelphia, PA, Pharmaceutical Press, 2012; Pharmaceutical Dosage Forms (Vol. 1-3), Liberman et al., Eds., Marcel Dekker, New York, NY, 1992; Handbook of Pharmaceutical Excipients (3rd Ed.), edited by Arthur H. Kibbe, American Pharmaceutical Association, Washington, 2000; Pharmaceutical Formulation: The Science and Technology of Dosage Forms (Drug Discovery), first edition, edited by GD Tovey, Royal Society of Chemistry, 2018. In some cases, the pharmaceutical composition described herein comprises a therapeutically effective amount of a compound disclosed herein, or a pharmaceutically acceptable salt thereof.

The compound(s) disclosed herein may be administered by any suitable route in the form of a pharmaceutical composition adapted to such a route and in a dose effective for the treatment intended. The compounds and compositions presented herein may, for example, be administered orally, mucosally, topically, transdermally, rectally, pulmonarily, parentally, intranasally, intravascularly, intravenously, intraarterial, intraperitoneally, intrathecally, subcutaneously, sublingually, intramuscularly, intrasternally, vaginally or by infusion techniques, in dosage unit formulations containing conventional pharmaceutically acceptable excipients.

The pharmaceutical composition may be in the form of, for example, a tablet, chewable tablet, minitablet, caplet, pill, bead, hard capsule, soft capsule, gelatin capsule, granule, powder, lozenge, patch, cream, gel, sachet, microneedle array, syrup, flavored syrup, juice, drop, injectable solution, emulsion, microemulsion, ointment, aerosol, aqueous suspension, or oily suspension. In some cases, the pharmaceutical composition is made in the form of a dosage unit containing a particular amount of the active ingredient.

Thus, a further aspect of the disclosure is a pharmaceutical composition comprising one or more of the compounds disclosed herein, or a pharmaceutically acceptable salt thereof, and a pharmaceutically acceptable excipient. Further provided herein is a compound of the disclosure, or a pharmaceutically acceptable salt thereof, or the pharmaceutical composition described herein, for us as a medicament.

### Methods of Use

The compounds described herein can covalently bind to cysteine-12 of the GDP-bound form of the G12C-mutant KRAS protein (“KRAS^{G12C}”). In some cases, the compounds described herein can act as potent inhibitors of KRAS^{G12C} by, for example, permanently inactivating the protein. Without intending to be bound by any particular theory, the compounds of the disclosure can, in some cases, inhibit phosphorylation of extracellular signal-regulated (“ERK”), which is a key down-stream effector of KRAS, leading to tumor regression. Besides being useful for human treatment, the compounds provided herein may be useful for veterinary treatment of companion animals, exotic animals, and farm animals, including mammals, rodents, and the like. For example, animals including horses, dogs, and cats may be treated with compounds provided herein.

### Monotherapy

Another aspect of the disclosure provides methods of using the compounds disclosed herein, or pharmaceutically acceptable salts thereof, or the pharmaceutical compositions of the present disclosure to treat disease conditions, including but not limited to conditions implicated by KRAS G12C mutation (e.g., cancer). See, e.g., U.S. Pat. No. 10,519,146 B2, issued Dec. 31, 2019; specifically, the section from column 198, line 1, to column 201, line 36, which is herewith incorporated by reference.

Without wishing to be bound by any particular theory, the following is noted: sotorasib is a small molecule that—similarly to the compounds disclosed herein—specifically and irreversibly inhibits KRAS^{G12C} (see Hong et al., N. Engl. J. Med. 2020, 383, 1207, at 1208). Hong et al. report that “[p]reclinical studies showed that [sotorasib] inhibited nearly all detectable phosphorylation of extracellular signal-regulated kinase (ERK), a key down-stream effector of KRAS, leading to durable complete tumor regression in mice bearing KRAS p.G12C tumors.” (id., see also Section entitled “BIOLOGICAL EVALUATION” below, Canon et al., Nature 2019, 575(7781), 217; and Lanman et al., J. Med. Chem. 2020, 63, 52).

Sotorasib was evaluated in a Phase 1 dose escalation and expansion trial with 129 subjects having histologically confirmed, locally advanced or metastatic cancer with the KRAS G12C mutation identified by local molecular testing on tumor tissues, including 59 subjects with non-small cell lung cancer, 42 subjects with colorectal cancer, and 28 subjects with other tumor types (Hong et al., 2020, at page 1208-1209). Hong et al. report a disease control rate (95% Cl) of 88.1% for non-small cell lung cancer. 73.8% for colorectal cancer and 75.0% for other tumor types (Hong et al., 2020, at page 1213, Table 3). The cancer types showing either stable disease (SD) or partial response (PR) as reported by Hong et al. were non-small cell lung cancer, colorectal cancer, pancreatic cancer, appendiceal cancer, endometrial cancer, esophageal cancer, cancer of unknown primary, ampullary cancer, gastric cancer, small bowel cancer, sinonasal cancer, bile duct cancer, or melanoma (Hong et al., 2020, at page 1212 (Figure A), and Supplementary Appendix (page 59 (Figure S5) and page 63 (Figure S6)).

KRASG12C mutations occur with the alteration frequencies shown in the table below (Cerami et al., Cancer Discov. 2012, 2(5), 401; Gao et al., Science Signaling 2013, 6(269), p 11). For example, the table shows that 11.6% of subjects with non-small cell lung cancer have a cancer, wherein one or more cells express KRAS G12C mutant protein. Accordingly, the compounds provided herein, which specifically and irreversibly bind to KRAS^{G12C} (see Section entitled “BIOLOGICAL EVALUATION” below), are useful for treatment of subjects having a cancer, including, but not limited to the cancers listed in the table below.

| Cancer Type | Alteration Frequency |
|---|---|
| Non-Small Cell Lung Cancer | 11.6 |
| Small Bowel Cancer | 4.2 |
| Appendiceal Cancer | 3.6 |
| Colorectal Cancer | 3.0 |
| Cancer of Unknown Primary | 2.9 |
| Endometrial Cancer | 1.3 |
| Mixed Cancer Types | 1.2 |
| Pancreatic Cancer | 1.0 |
| Hepatobiliary Cancer | 0.7 |
| Small Cell Lung Cancer | 0.7 |
| Cervical Cancer | 0.7 |
| Germ Cell Tumor | 0.6 |
| Ovarian Cancer | 0.5 |
| Gastrointestinal Neuroendocrine Tumor | 0.4 |
| Bladder Cancer | 0.4 |
| Myelodysplastic/Myeloproliferative Neoplasms | 0.3 |
| Head and Neck Cancer | 0.3 |
| Esophagogastric Cancer | 0.2 |
| Soft Tissue Sarcoma | 0.2 |
| Mesothelioma | 0.2 |
| Thyroid Cancer | 0.1 |
| Leukemia | 0.1 |
| Melanoma | 0.1 |

Another aspect of the disclosure provides a compound disclosed herein (e.g., a compound of Formula (I), Formula (I′), Formula (IA), Formula (IB), Formula (IE), Formula (IF), Formula (IG), Formula (II), Formula (II′), Formula (IIA), Formula (IIB), Formula (IIC), Formula (IID), Formula (IIE), and Formula (IIF), or a compound listed in Table A, Table A′, Table B, Table B′, or Table E)), and pharmaceutically acceptable salts thereof, or a pharmaceutical composition disclosed herein, for use in treating cancer. Yet another aspect of the disclosure provides a compound disclosed herein, or a pharmaceutically acceptable salt thereof, or a pharmaceutical composition disclosed herein, for use in treating cancer, wherein one or more cells express KRAS G12C mutant protein.

Another aspect of the disclosure provides a compound disclosed herein, or a pharmaceutically acceptable salt thereof, or a pharmaceutical composition disclosed herein, in the preparation of a medicament for treating cancer. Yet another aspect of the disclosure provides a compound disclosed herein, or a pharmaceutically acceptable salt thereof, or a pharmaceutical composition described herein, in the preparation of a medicament for treating cancer, wherein one or more cells express KRAS G12C mutant protein.

A further aspect provided by the disclosure is a method of treating cancer in a subject in need thereof, the method comprising administering to the subject a therapeutically effective amount of a compound disclosed herein, or a pharmaceutically acceptable salt thereof, or a pharmaceutical composition disclosed herein. Another aspect of the disclosure is a method of treating cancer in a subject in need thereof, the method comprising administering to the subject a therapeutically effective amount of a compound disclosed herein, or a pharmaceutically acceptable salt thereof, or a pharmaceutical composition of the disclosure, wherein one or more cells express KRAS G12C mutant protein. In some cases, the subject has a cancer that was determined to have one or more cells expressing the KRAS G12C mutant protein prior to administration of the compound or a pharmaceutically acceptable salt thereof.

In some cases, the cancer is metastatic. In some cases, the cancer is non-metastatic. In some cases, the cancer disclosed herein is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, or a solid tumor. In some cases, the cancer is non-small cell lung cancer, colorectal cancer, pancreatic cancer, appendiceal cancer, endometrial cancer, esophageal cancer, cancer of unknown primary, ampullary cancer, gastric cancer, small bowel cancer, sinonasal cancer, bile duct cancer, melanoma, or a solid tumor. In some cases, the cancer is non-small cell lung cancer. In some cases, the cancer is colorectal cancer. In some cases, the cancer is pancreatic cancer. In some cases, the cancer is solid tumor.

### Combination Therapy

The present disclosure also provides methods for combination therapies in which an agent known to modulate other pathways, or other components of the same pathway, or even overlapping sets of target enzymes are used in combination with a compound of the present disclosure (e.g., a compound of Formula (I), Formula (I′), Formula (IA), Formula (IB), Formula (IE), Formula (IF), Formula (IG), Formula (II), Formula (II′), Formula (IIA), Formula (IIB), Formula (IIC), Formula (UD), Formula (IIE), or Formula (IIF), or a compound listed in Table A, Table A′, Table B, Table B″, or Table E), or a pharmaceutically acceptable salt thereof. In one aspect, such therapy includes but is not limited to the combination of one or more compounds of the disclosure with chemotherapeutic agents, therapeutic antibodies, and/or radiation treatment, to provide a synergistic or additive therapeutic effect. See, e.g., U.S. Pat. No. 10,519,146 B2, issued Dec. 31, 2019; specifically, the sections from column 201 (line 37) to column 212 (line 46) and column 219 (line 64) to column 220 (line 39), which are herewith incorporated by reference.

The compounds of the disclosure can be administered simultaneously, separately, or sequentially with an effective amount of a second compound in any of the methods described herein. In some cases, the second compound is an ATR inhibitor, Aurora kinase A inhibitor, AKT inhibitor, arginase inhibitor, CDK2 inhibitor, CDK4/6 inhibitor, ErbB family inhibitor, ERK inhibitor, FAK inhibitor, FGFR inhibitor, glutaminase inhibitor, IGF-IR inhibitor, KIF18A inhibitor, MAT2A inhibitor, MCL-1 inhibitor, MEK inhibitor, mTOR inhibitor, PARP inhibitor, PD-1 inhibitor, PD-L1 inhibitor, PI3K inhibitor, PRMT5 inhibitor, Raf kinase inhibitor, SHP2 inhibitor, SOS1 inhibitor, Src kinase inhibitor, or one or more chemotherapeutic agents. In some cases, the second compound is administered as a pharmaceutically acceptable salt. In some cases, the second compound is administered as a pharmaceutical composition comprising the second compound or a pharmaceutically acceptable salt thereof and a pharmaceutically acceptable excipient.

ATR inhibitors. In some cases, the compounds of the disclosure can be administered simultaneously, separately, or sequentially with an effective amount of an ATR inhibitor in any of the methods described herein. An ATR inhibitor is a compound that targets the ataxia telangiectasia mutated and Rad3-related kinase. Exemplary ATR inhibitors for use in the methods provided herein include, but are not limited to dactolisib, VE-821 (3-Amino-6-(4-(methylsulfonyl)phenyl)-N-phenylpyrazine-2-carboxamide, 3-Amino-6-[4-(methylsulfonyl)phenyl]-N-phenyl-2-pyrazinecarboxamide), Torin 2 (9-(6-amino-3-pyridinyl)-1-[3-(trifluoromethyl)phenyl]-benzo[b]-1,6-naphthyridin-2 (1H)-one), ETP-46464 (α,α-dimethyl-4-[2-oxo-9-(3-quinolinyl)-2H-[1,3]oxazino[5,4198zetidinelin-1(4H)-yl]-benzeneacetonitrile), CGK 733 (α-Phenyl-N-[2,2,2-trichloro-1-[[[(4-fluoro-3-nitrophenyl)amino]thioxomethyl]amino]ethyl]benzeneacetamide), AZ20 (4-[4-[(3R)-3-Methyl-4-morpholinyl]-6-[1-(methylsulfonyl)cyclopropyl]-2-pyrimidinyl]-1H-indole), SKLB-197 ((R)-4-(2-(1H-indol-4-yl)-6-(1-methyl-1H-pyrazol-5-yl) quinazolin-4-yl)-3-methylmorpholine), elimusertib, gartisertib, elimusertib hydrochloride, ceralasertib, and schisandrin B.

Aurora Kinase A Inhibitors. In some cases, the compounds of the disclosure can be administered simultaneously, separately, or sequentially with an effective amount of an Aurora kinase A inhibitor in any of the methods described herein. Exemplary Aurora kinase A inhibitors for use in the methods provided herein include, but are not limited to, alisertib, cenisertib, danusertib, tozasertib, LY3295668 ((2R,4R)-1-[(3-chloro-2-fluorophenyl)methyl]-4-[[3-fluoro-6-[(5-methyl-1H-pyrazol-3-yl)amino]pyridin-2-yl]methyl]-2-methylpiperidine-4-carboxylic acid), ENMD-2076 (6-(4-methylpiperazin-1-yl)-N-(5-methyl-1H-pyrazol-3-yl)-2-[(E)-2-phenylethenyl]pyrimidin-4-amine), TAK-901 (5-(3-ethylsulfonylphenyl)-3,8-dimethyl-N-(1-methylpiperidin-4-yl)-9H-pyrido[2,3-b]indole-7-carboxamide), TT-00420 (4-[9-(2-chlorophenyl)-6-methyl-2,4,5,8,12-pentazatricyclo[8.4.0.03,7]tetradeca-1(14),3,6,8,10,12-hexaen-13-yl]morpholine), AMG 900 (N-[4-[3-(2-aminopyrimidin-4-yl)pyridin-2-yl]oxyphenyl]-4-(4-methylthiophen-2-yl)phthalazin-1-amine), MLN8054 (4-[[9-chloro-7-(2,6-difluorophenyl)-5H-pyrimido[5,4-d][2]benzazepin-2-yl]amino]benzoic acid), PF-03814735 (N-[2-[(1R,8S)-4-[[4-(cyclobutylamino)-5-(trifluoromethyl)pyrimidin-2-yl]amino]-11-azatricyclo[6.2.1.02,7]undeca-2(7),3,5-trien-11-yl]-2-oxoethyl]acetamide), SNS-314 (1-(3-chlorophenyl)-3-[5-[2-(thieno[3,2-d]pyrimidin-4-ylamino)ethyl]-1,3-thiazol-2-ylurea), CYC116 (4-methyl-5-[2-(4-morpholin-4-ylanilino)pyrimidin-4-yl]-1,3-thiazol-2-amine), TAS-119, BI 811283, and TTP607.

AKT Inhibitors. In some cases, the compounds of the disclosure can be administered simultaneously, separately, or sequentially with an effective amount of an AKT inhibitor in any of the methods described herein. Exemplary AKT inhibitors for use in the methods provided herein include, but are not limited to, afuresertib, capivasertib, ipatasertib, uprosertib, BAY1125976 (2-[4-(1-aminocyclobutyl)phenyl]-3-phenylimidazo[1,2-b]pyridazine-6-carboxamide), ARQ 092 (3-[3-[4-(1-aminocyclobutyl)phenyl]-5-phenylimidazo[4,5-b]pyridin-2-yl]pyridin-2-amine), MK2206 (8-[4-(1-aminocyclobutyl)phenyl]-9-phenyl-2H-[1,2,4]triazolo[3,4-f][1,6]naphthyridin-3-one), SR13668 (indolo[2,3-b]carbazole-2,10-dicarboxylic acid, 5,7-dihydro-6-methoxy-, 2,10-diethyl ester), ONC201 (11-benzyl-7-[(2-methylphenyl)methyl]-2,5,7,11-tetrazatricyclo[7.4.0.02,6]trideca-1 (9),5-dien-8-one), ARQ 751 (N-(3-aminopropyl)-N-[(1R)-1-(3-anilino-7-chloro-4-oxoquinazolin-2-yl) but-3-ynyl]-3-chloro-2-fluorobenzamide), RX-0201, and LY2780301.

Arginase Inhibitors. In some cases, the compounds of the disclosure can be administered simultaneously, separately, or sequentially with an effective amount of an arginase inhibitor in any of the methods described herein. Exemplary arginase inhibitors for use in the methods provided herein include, but are not limited to, numidargistat and CB 280.

CDK 2 Inhibitors. In some cases, the compounds of the disclosure can be administered simultaneously, separately, or sequentially with an effective amount of a CDK 2 inhibitor in any of the methods described herein. The term “CDK 2” as used herein refers to cyclin dependent kinases (“CDK”) 2, which is a member of the mammalian serine/threonine protein kinases. The term “CDK 2 inhibitor” as used herein refers to a compound that is capable of negatively modulating or inhibiting all or a portion of the enzymatic activity of CDK 2. Exemplary CDK 2 inhibitors for use in the methods provided herein include, but are not limited to, flavopiridol, roscovitine, dinaciclib, milciclib, meriolin, variolin, AZD5438 (4-[2-Methyl-1-(1-methylethyl)-1H-imidazol-5-yl]-N-[4-(methylsulfonyl)phenyl]-2-pyrimidinamine), roniciclib, SNS-032 (N-[5-[[[5-(1,1-Dimethylethyl)-2-oxazolyl]methyl]thio]-2-thiazolyl]-4-piperidinecarboxamide).

CDK4/6 Inhibitors. In some cases, the compounds of the disclosure can be administered simultaneously, separately, or sequentially with an effective amount of a CDK4/6 inhibitor in any of the methods described herein. The term “CDK 4/6” as used herein refers to cyclin dependent kinases (“CDK”) 4 and 6, which are members of the mammalian serine/threonine protein kinases. The term “CDK 4/6 inhibitor” as used herein refers to a compound that is capable of negatively modulating or inhibiting all or a portion of the enzymatic activity of CDK 4 and/or 6. Exemplary CDK 4/6 inhibitors for use in the methods provided herein include, but are not limited to, abemaciclib, palbociclib, ribociclib, trilaciclib, and PF-06873600 ((pyrido[2,3-d]pyrimidin-7 (8H)-one, 6-(difluoromethyl)-8-[(1R,2R)-2-hydroxy-2-methylcyclopentyl]-2-[[1-(methylsulfonyl)-4-piperidinyl]amino]). In some cases, the CDK4/6 inhibitor is palbociclib.

ErbB Family Inhibitors. In some cases, the compounds of the disclosure can be administered simultaneously, separately, or sequentially with an effective amount of an ErbB family inhibitor in any of the methods described herein. The term “ErbB family” as used herein refers to a member of a mammalian transmembrane protein tyrosine kinase family including: ErbB1 (EGFR HER1), ErbB2 (HER2), ErbB3 (HER3), and ErbB4 (HER4). The term “ErbB family inhibitor” as used herein refers to an agent, e.g., a compound or antibody, that is capable of negatively modulating or inhibiting all or a portion of the activity of at least one member of the ErbB family. The modulation or inhibition of one or more ErbB tyrosine kinase may occur through modulating or inhibiting kinase enzymatic activity of one or more ErbB family member or by blocking homodimerization or heterodimerization of ErbB family members. In some cases, the ErbB family inhibitor is an EGFR inhibitor, e.g., an anti-EGFR antibody. Exemplary anti-EGFR antibodies for use in the methods provided herein include, but are not limited to, zalutumumab, nimotuzumab, matuzumab, necitumumab, panitumumab, and cetuximab. In some cases, the anti-EGFR antibody is cetuximab. In some cases, the anti-EGFR antibody is panitumumab. In some cases, the ErbB family inhibitor is a HER2 inhibitor, e.g., an anti-HER2 antibody. Exemplary anti-HER-2 antibodies for use in the methods provided herein include, but are not limited to, pertuzumab, trastuzumab, and trastuzumab emtansine. In some cases, the ErbB family inhibitor is a HER3 inhibitor, e.g., an anti-HER3 antibody, such as HMBD-001 (Hummingbird Bioscience). In some cases, the ErbB family inhibitor is a combination of an anti-EGFR antibody and anti-HER2 antibody. In some cases, the ErbB family inhibitor is an irreversible inhibitor. Exemplary irreversible ErbB family inhibitors for use in the methods provided herein include, but are not limited to, afatinib, dacomitinib, canertinib, poziotinib, AV 412 ((N-[4-[(3-chloro-4-fluorophenyl)amino]-7-[3-methyl-3-(4-methyl-1-piperazinyl)-1-butyn-1-yl]-6-quinazolinyl]-2-propenamide)), PF 6274484 ((N-[4-[(3-chloro-4-fluorophenyl)amino]-7-methoxy-6-quinazolinyl]-2-propenamide), and HKI 357 ((E)-N-[4-[3-chloro-4-[(3-fluorophenyl)methoxy]anilino]-3-cyano-7-ethoxy quinolin-6-yl]-4-(dimethylamino)but-2-enamide). In some cases, the irreversible ErbB family inhibitor is afatinib. In some cases, the irreversible ErbB family inhibitor is dacomitinib. In some cases, the ErbB family inhibitor is a reversible inhibitor. Exemplary reversible ErbB family inhibitors for use in the methods provided herein include, but are not limited to erlotinib, gefitinib, sapitinib, varlitinib, tarloxotinib, TAK-285 (N-(2-(4-(3-chloro-4-(3-(trifluoromethyl) phenoxy)phenyl)amino)-5H-pyrrolo[3,2-d]pyrimidin-5-yl)ethyl)-3-hydroxy-3-methylbutanamide), AEE788 (S)-6-(4-((4-ethylpiperazin-1-yl)methyl)phenyl)-N-(1-phenylethyl)-7H-pyrrolo[2,3-d]pyrimidin-4-amine), BMS 599626 ((3S)-3-morpholinylmethyl-[4-[[1-[(3-fluorophenyl methyl]-1H-indazol-5-yl]amino]-5-methylpyrrolo[2,1-f][1,2,4]triazin-6-yl]-carbamate), and GW 583340 (N-[3-chloro-4-[(3-fluorophenyl)methoxy]phenyl]-6-[2-[(2-methylsulfonylethylamino)methyl]-1,3-thiazol-4-yl]quinazolin-4-amine). In some cases, the reversible ErbB family inhibitor is sapitinib. In one embodiment, the reversible ErbB family inhibitor is tarloxotinib.

ERK Inhibitors. In some cases, the compounds of the disclosure can be administered simultaneously, separately, or sequentially with an effective amount of an ERK inhibitor in any of the methods described herein. Exemplary ERK inhibitors for use in the methods provided herein include, but are not limited to, ulixertinib, ravoxertinib, CC-90003 (N-[2-[[2-[(2-methoxy-5-methylpyridin-4-yl)amino]-5-(trifluoromethyl)pyrimidin-4-yl]amino]-5-methylphenyl]prop-2-enamide), LY3214996 (6,6-dimethyl-2-[2-[(2-methylpyrazol-3-yl)amino]pyrimidin-4-yl]-5-(2-morpholin-4-ylethyl) thieno[2,3-c]pyrrol-4-one), KO-947 (1,5,6,8-tetrahydro-6-(phenylmethyl)-3-(4-pyridinyl)-7H-pyrazolo[4,3-g]quinazolin-7-one), ASTX029, LTT462, and JSI-1187,

FAK Inhibitors. In some cases, the compounds of the disclosure can be administered simultaneously, separately, or sequentially with an effective amount of a FAK inhibitor in any of the methods described herein. Exemplary FAK inhibitors for use in the methods provided herein include, but are not limited to, GSK2256098 (2-[[5-chloro-2-[(5-methyl-2-propan-2-ylpyrazol-3-yl)amino]pyridin-4-yl]amino]-N-methoxybenzamide), PF-00562271 (N-methyl-N-[3-[[[2-[(2-oxo-1,3-dihydroindol-5-yl)amino]-5-(trifluoromethyl)pyrimidin-4-yl]amino]methyl]pyridin-2-yl]methanesulfonamide), VS-4718 (2-[[2-(2-methoxy-4-morpholin-4-ylanilino)-5-(trifluoromethyl)pyridin-4-yl]amino]-N-methylbenzamide), and APG-2449.

FGFR Inhibitors. In some cases, the compounds of the disclosure can be administered simultaneously, separately, or sequentially with an effective amount of an FGFR inhibitor in any of the methods described herein. Exemplary FGFR inhibitors for use in the methods provided herein include, but are not limited to, futibatinib, pemigatinib, ASP5878 (2-[4-[[5-[(2,6-difluoro-3,5-dimethoxyphenyl)methoxy |pyrimidin-2-yl]amino]pyrazol-1-yl]ethanol), AZD4547 (N-[5-[2-(3,5-dimethoxyphenyl)ethyl]-1H-pyrazol-3-yl]-4-[(3S,5R)-3,5-dimethylpiperazin-1-yl]benzamide), debio 1347 ([5-amino-1-(2-methyl-3H-benzimidazol-5-yl) pyrazol-4-yl]-(1H-indol-2-yl) methanone), INCB062079, H3B-6527 (N-[2-[[6-[(2,6-dichloro-3,5-dimethoxyphenyl)carbamoyl-methylamino]pyrimidin-4-yl]amino]-5-(4-ethylpiperazin-1-yl)phenyl]prop-2-enamide), ICP-105, CPL304110, HMPL-453, and HGS1036.

Glutaminase Inhibitors. In some cases, the compounds of the disclosure can be administered simultaneously, separately, or sequentially with an effective amount of a glutaminase inhibitor in any of the methods described herein. Exemplary glutaminase inhibitors for use in the methods provided herein include, but are not limited to, telaglenastat, IPN60090, and OP 330.

IGF-IR Inhibitors. In some cases, the compounds of the disclosure can be administered simultaneously, separately, or sequentially with an effective amount of an IGF-IR inhibitor in any of the methods described herein. Exemplary IGF-IR inhibitors for use in the methods provided herein include, but are not limited to, cixutumumab, dalotuzumab, linsitinib, ganitumab, robatumumab, BMS-754807 ((2S)-1-[4-[(5-cyclopropyl-1H-pyrazol-3-yl)amino]pyrrolo[2,1-f][1,2,4]triazin-2-yl]-N-(6-fluoropyridin-3-yl)-2-methylpyrrolidine-2-carboxamide), KW-2450 (N-[5-[[4-(2-hydroxyacetyl)piperazin-1-yl]methyl]-2-[(E)-2-(1H-indazol-3-yl) ethenyl]phenyl]-3-methylthiophene-2-carboxamide), PL225B, AVE1642, and BIIB022.

KIF18A Inhibitors. In some cases, the compounds of the disclosure can be administered simultaneously, separately, or sequentially with an effective amount of a KIF18A inhibitor in any of the methods described herein. Exemplary KIF18A inhibitors for use in the methods provided herein include, but are not limited to, the inhibitors disclosed in US 2020/0239441, WO 2020/132649, WO 2020/132651, and WO 2020/132653, each of which is herewith incorporated by reference in its entirety. In some cases, the KIF18A inhibitor is sovilnesib (AMG 650).

MAT2A inhibitors. In some cases, the compounds of the disclosure can be administered simultaneously, separately, or sequentially with an effective amount of a MAT2A inhibitor in any of the methods described herein. An MAT2A inhibitor is a compound that inhibits methionine adenosyltransferase II alpha. An exemplary MAT2A inhibitor for use in the methods provided herein is AG 270 (3-(cyclohex-1-en-1-yl)-6-(4-methoxyphenyl)-2-phenyl-202zetidindin-2-ylamino)pyrazolo[1,5-a]pyrimidin-7 (4H)-one).

MCL-1 Inhibitors. In some cases, the compounds of the disclosure can be administered simultaneously, separately, or sequentially with an effective amount of a MCL-1 inhibitor in any of the methods described herein. Exemplary MCL-1 inhibitors for use in the methods provided herein include, but are not limited to, murizatoclax, tapotoclax, AZD 5991 ((3aR)-5-chloro-2,11,12,24,27,29-hexahydro-2,3,24,33-tetramethyl-22H-9,4,8-(metheniminomethyno)-14,20:26,23-dimetheno-10H,20H-pyrazolo[4,3-1][2,15,22,18,19]benzoxadithiadiazacyclohexacosine-32-carboxylic acid). MIK 665 (αR)-α-[[(5S)-5-[3-Chloro-2-methyl-4-[2-(4-methyl-]-piperazinyl) ethoxy |phenyl]-6-(4-fluorophenyl) thieno[2,3-d]pyrimidin-4-yl]oxy]-2-[[2-(2-methoxyphenyl)-4-pyrimidinyl]methoxy]benzenepropanoic acid), and ABBV-467. In some cases, the MCL-1 inhibitor is murizatoclax. In some cases, the MCL-1 inhibitor is tapotoclax.

MEK Inhibitors. In some cases, the compounds of the disclosure can be administered simultaneously, separately, or sequentially with an effective amount of a MEK inhibitor in any of the methods described herein. Exemplary MEK inhibitors for use in the methods provided herein include, but are not limited to, trametinib, cobimctinib, selumetinib, pimasertib, refametinib, PD-325901 (N-[(2R)-2,3-dihydroxypropoxy]-3,4-difluoro-2-(2-fluoro-4-iodoanilino)benzamide), AZD8330 (2-(2-fluoro-4-iodoanilino)-N-(2-hydroxyethoxy)-1,5-dimethyl-6-oxopyridine-3-carboxamide), GDC-0623 (5-(2-fluoro-4-iodoanilino)-N-(2-hydroxyethoxy) imidazo[1,5-a]pyridine-6-carboxamide), RO4987655 (3,4-difluoro-2-(2-fluoro-4-iodoanilino)-N-(2-hydroxyethoxy)-5-[(3-oxooxazinan-2-yl)methyl]benzamide), TAK-733 (3-[(2R)-2,3-dihydroxypropyl]-6-fluoro-5-(2-fluoro-4-iodoanilino)-8-methylpyrido[2,3-d]pyrimidine-4,7-dione), PD0325901 (N-[(2R)-2,3-dihydroxypropoxy]-3,4-difluoro-2-(2-fluoro-4-iodoanilino)benzamide), CI-1040 (2-(2-chloro-4-iodophenylamino)-N-(cyclopropylmethoxy)-3,4-difluorobenzamide), PD318088 (5-bromo-N-(2,3-dihydroxypropoxy)-3,4-difluoro-2-(2-fluoro-4-iodophenylamino)benzamide), PD98059 (2-(2-amino-3-methoxyphenyl)-4H-chromen-4-one), PD334581 (N-[5-[3,4-Difluoro-2-[(2-fluoro-4-iodophenyl)amino]phenyl]-1,3,4-oxadiazol-2-yl]-4-morpholineethanamine), FCN-159, CS3006, HL-085, SHR 7390, and WX-554. In some cases, the MEK inhibitor is trametinib.

mTOR Inhibitors. In some cases, the compounds of the disclosure can be administered simultaneously, separately, or sequentially with an effective amount of a mTOR inhibitor in any of the methods described herein. Exemplary mTOR inhibitors for use in the methods provided herein include, but are not limited to, everolimus, rapamycin, zotarolimus (ABT-578), ridaforolimus (deforolimus, MK-8669), sapanisertib, buparlisib, pictilisib, vistusertib, dactolisib, Torin-1 (1-(4-(4-propionylpiperazin-1-yl)-3-(trifluoromethyl)cyclohexyl)-9-(quinolin-3-yl)benzo[h][1,6]naphthyridin-2(1H)-one), GDC-0349 ((S)-1-ethyl-3-(4-(4-(3-methylmorpholino)-7-(oxetan-3-yl)-5,6,7,8-tetrahydropyrido[3,4-d]pyrimidin-2-yl)phenyl)urea), and VS-5584 (SB2343, (5-(8-methyl-2-morpholin-4-yl-9-propan-2-ylpurin-6-yl)pyrimidin-2-amine). In some cases, the mTOR inhibitor is everolimus.

PARP inhibitors. In some cases, the compounds of the disclosure can be administered simultaneously, separately, or sequentially with an effective amount of a PARP inhibitor in any of the methods described herein. A PARP inhibitor is a compound that targets poly(adenosine diphosphate)-ribose polymerase. The term PARP inhibitors encompasses PARP1, PARP2, and PARP3 inhibitors. Exemplary PARP inhibitors for use in the methods provided herein include, but are not limited t203zetidinrib, rucaparib, rucaparib camsylate, niraparib, niraparib tosylate, talazoparib, AG-1461, A-966492, PJ34 HCL niraparib, UPF 1069, ME0328, venadaparib, AZD5305, DR2313, BYK204165, pamiparib, NMS-P118, and NU 1025.

PD-1 Inhibitors. In some cases, the compounds of the disclosure can be administered simultaneously, separately, or sequentially with an effective amount of a PD-1 inhibitor in any of the methods described herein. Exemplary PD-1 inhibitors for use in the methods provided herein include, but are not limited to, pembrolizumab, nivolumab, cemiplimab, spartalizumab (PDR001), camrelizumab (SHR1210), sintilimab (IBI308), tislelizumab (BGB-A317), toripalimab (JS 001), dostarlimab (TSR-042, WBP-285), INCMGA00012 (MGA012), AMP-224, AMP-514, and the anti-PD-1 antibody as described in U.S. Pat. No. 10,640,504 B2 (the “Anti-PD-1 Antibody A,” column 66, line 56 to column 67, line 24 and column 67, lines 54-57), which is incorporated herein by reference. In some cases, the PD-1 inhibitor is pembrolizumab. In some cases, the PD-1 inhibitor is the Anti-PD-1 Antibody A.

PD-L1 Inhibitors. In some cases, the compounds of the disclosure can be administered simultaneously, separately, or sequentially with an effective amount of a PD-L1 inhibitor in any of the methods described herein. Exemplary PD-L1 inhibitors for use in the methods provided herein include, but are not limited to, atezolizumab, avelumab, durvalumab, ZKAB001, TG-1501, SHR-1316, MSB2311, MDX-1105, KN035, IMC-001, HLX20, FAZ053, CS1001, CK-301, CBT-502, BGB-A333, BCD-135, and A167. In some cases, the PD-L1 inhibitor is atezolizumab.

PI3K Inhibitors. In some cases, the compounds of the disclosure can be administered simultaneously, separately, or sequentially with an effective amount of a PI3K inhibitor in any of the methods described herein. Exemplary PI3K inhibitors for use in the methods provided herein include, but are not limited to, idelalisib, copanlisib, duvelisib, alpelisib, taselisib, perifosine, buparlisib, umbralisib, pictilisib, dactolisib, voxtalisib, sonolisib, tenalisib, serabelisib, acalisib, CUDC-907 (N-hydroxy-2-[[2-(6-methoxypyridin-3-yl)-4-morpholin-4-ylthieno[3,2-d]pyrimidin-6-y]]methyl-methylamino]pyrimidine-5-carboxamide), ME-401 (N-[2-methyl-1-[2-(1-methylpiperidin-4-yl)phenyl]propan-2-yl]-4-(2-methylsulfonylbenzimidazol-1-yl)-6-morpholin-4-yl-1,3,5-triazin-2-amine), IPI-549 (2-amino-N-[(1S)-1-[8-[2-(1-methylpyrazol-4-yl)ethynyl]-1-oxo-2-phenylisoquinolin-3-yl]ethyl]pyrazolo[1,5-a]pyrimidine-3-carboxamide), SF1126 ((2S)-2-[(2S)-3-carboxy-2-[2-[[(2S)-5-(diaminomethylidencamino)-2-[[4-oxo-4-[[4-(4-oxo-8-phenylchromen-2-yl)morpholin-4-ium-4-yl]methoxy]butanoyl]amino]pentanoyl]amino]acetyl]amino]propanoyl]amino]-3-hydroxypropanoate). XL147 (N-[3-(2, 1,3-benzothiadiazol-5-ylamino) quinoxalin-2-yl]-4-methylbenzenesulfonamide), GSK1059615 ((5Z)-5-[(4-pyridin-4-ylquinolin-6-yl)methylidene]-1,3-thiazolidine-2,4-dione), and AMG 319 (N-[(1S)-1-(7-fluoro-2-pyridin-2-ylquinolin-3-yl)ethyl]-7H-purin-6-amine).

PRMT5 Inhibitors. In some cases, the compounds of the disclosure can be administered simultaneously, separately, or sequentially with an effective amount of a PRMT5 inhibitor in any of the methods described herein. A PRMT5 inhibitor in a compound that inhibits protein arginine methyltransferase 5. The term “PRMT5 inhibitor” includes MTA-cooperative PRMT5 inhibitors, Exemplary PRMT5 inhibitors for use in the methods provided herein include, but are not limited to, pemrametostat (6-[(1-acetylpiperidin-4-yl)amino]-N-[(2S)-3-(3,4-dihydro-1H-isoquinolin-2-yl)-2-hydroxypropyl]pyrimidine-4-carboxamide), GSK3203591 (2-(Cyclobutylamino)-N-[(2S)-3-(3,4-dihydro-2 (1H)-isoquinolinyl)-2-hydropropyl]-4-pyridinecarboxamide dihydrochloride)), LLY-283 ((R)-5′-phenyl-7-deazaadenosine; 6-amino-9-| (R)-5′-phenyl(ribofuranosyl) |-7-deazapurine, (2R,3R,4S,5R)-2-(4-Amino-7H-pyrrolo[2,3-d]pyrimidin-7-yl)-5-((R)-hydroxy (phenyl)methyl)tetrahydrofuran-3,4-diol), PRT 811, and MRTX1719 (2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile).

Raf Kinase Inhibitors. In some cases, the compounds of the disclosure can be administered simultaneously, separately, or sequentially with an effective amount of a Raf kinase inhibitor in any of the methods described herein. The term “RAF kinase” as used herein refers to a member of a mammalian serine/threonine kinases composed of three isoforms (C-Raf, B-Raf and A-Raf) and includes homodimers of each isoform as well as heterodimers between isoforms, e.g., C-Raf/B-Raf heterodimers. The term “Raf kinase inhibitor” as used herein refers to a compound that is capable of negatively modulating or inhibiting all or a portion of the enzymatic activity of one or more member of the Raf family kinases, or is capable of disrupting Raf homodimer or heterodimer formation to inhibit activity. In some cases, the Raf kinase inhibitor includes, but is not limited to, encorafenib, sorafenib, lifirafenib, vemurafenib, dabrafenib, PLX-8394 (N-(3-(5-(2-cyclopropylpyrimidin-5-yl)-3a, 7a-dihydro-1H-pyrrolo[2,3-b]pyridine-3-carbonyl)-2,4-difluorophenyl)-3-fluoropyrrolidine-1-sulfonamide), Raf-709 (N-(2-methyl-5, -morpholino-6′-((tetrahydro-2H-pyran-4-yl)oxy)-[3,3′-bipyridin]-5-yl)-3-(trifluoromethyl)benzamide), LXH254 (N-(3-(2-(2-hydroxyethoxy)-6-morpholinopyridin-4-yl)-4-methylphenyl)-2-(trifluoromethyl)isonicotinamide), LY3009120 (1-(3,3-dimethylbutyl)-3-(2-fluoro-4-methyl-5-(7-methyl-2-(methylamino)pyrido[2,3-d]pyrimidin-6-yl)phenyl)urea), Tak-632 (N-(7-cyano-6-(4-fluoro-3-(2-(3-(trifluoromethyl)phenyl) acetamido) phenoxy)benzo[d]thiazol-2-yl)cyclopropanecarboxamide), CEP-32496 (1-(3-((6,7-dimethoxyquinazolin-4-yl)oxy)phenyl)-3-(5-(1,1,1-trifluoro-2-methylpropan-2-yl)isoxazol-3-yl)urea), CCT196969 (1-(3-(tert-butyl)-1-phenyl-1H-pyrazol-5-yl)-3-(2-fluoro-4-((3-oxo-3,4-dihydropyrido[2,3-b]pyrazin-8-yl)oxy)phenyl)urea), and RO5126766 (N-[3-fluoro-4-[[4-methyl-2-oxo-7-(2-pyrimidinyloxy)-2H-1-benzopyran-3-yl|methyl]-2-pyridiny]]-N′-methyl-sulfamide). In some cases, the Raf kinase inhibitor is encorafenib. In some cases, the Raf kinase inhibitor is sorafenib. In some cases, the Raf kinase inhibitor is lifirafenib.

SHP2 Inhibitors. In some cases, the compounds of the disclosure can be administered simultaneously, separately, or sequentially with an effective amount of a SHP2 inhibitor in any of the methods described herein. Exemplary SHP2 inhibitors for use in the methods provided herein include, but are not limited to, SHP-099 (6-(4-amino-4-methylpiperidin-1-yl)-3-(2,3-dichlorophenyl) pyrazin-2-amine dibydrochloride), RMC-4550 ([3-[(3S,4S)-4-amino-3-methyl-2-oxa-8-azaspiro[4.5]decan-8-yl]-6-(2,3-dichlorophenyl)-5-methylpyrazin-2-yl]methanol), TNO155, (3S,4S)-8-[6-amino-5-(2-amino-3-chloropyridin-4-yl)sulfanylpyrazin-2-yl]-3-methyl-2-oxa-8-azaspiro[4.5]decan-4-amine), and RMC-4630 (Revolution Medicine; vociprotafib (RMC-4630; 6-[(2-amino-3-chloro-4-pyridinyl)thio]-3-[(3S,4S)-4-amino-3-methyl-2-oxa-8-azaspiro[4.5]dec-8-yl]-5-methyl-2-pyrazinemethanol). In some cases, the SHP inhibitor for use in the methods provided herein is RMC-4630 (vociprotafib, Revolution Medicine). In some cases, exemplary SHP2 inhibitors for use in the methods provided herein include, but are not limited to, 3-[(1R,3R)-1-amino-3-methoxy-8-azaspiro[4.5]dec-8-yl]-6-(2,3-dichlorophenyl)-5-methyl-2-pyrazinemethanol (CAS 2172651-08-8), 3-[(3S,4S)-4-amino-3-methyl-2-oxa-8-azaspiro[4,5]dec-8-y]]-6-[(2,3-dichlorophenyl)thio]-5-methyl-2-pyrazinemethanol (CAS 2172652-13-8), 3-[(3S,4S)-4-amino-3-methyl-2-oxa-8-azaspiro[4.5]dec-8-yl]-6-[[3-chloro-2-(3-hydroxy-1-azetidinyl)-4-pyridinyl]thio]-5-methyl-2-pyrazinemethanol (CAS 2172652-38-7), and 6-[(2-amino-3-chloro-4-pyridinyl)thio]-3-[(38,4S)-4-amino-3-methyl-2-oxa-8-azaspiro[4.5]dec-8-yl]-5-methyl-2-pyrazinemethanol (CAS 2172652-48-9). In some cases, exemplary SHP2 inhibitors for use in the methods provided herein include, but are not limited to, 1-[5-(2,3-dichlorophenyl)-6-methylimidazo[1,5-a]pyrazin-8-yl]-4-methyl-4-piperidinamine (CAS 2240981-75-1), (1R)-8-[5-(2,3-dichlorophenyl)-6-methylimidazo[1,5-a]pyrazin-8-y]]-8-azaspiro[4.5]decan-1-amine (CAS 2240981-78-4), (3S,4S)-8-[7-(2,3-dichlorophenyl)-6-methylpyrazolo[1,5-a]pyrazin-4-yl]-3-methyl-2-oxa-8-azaspiro[4.5]decan-4-amine (CAS 2240982-45-8), (3S,4S)-8-[7-[(2-amino-3-chloro-4-pyridinyl)thio]pyrazolo[1,5-a]pyrazin-4-y]]-3-methyl-2-oxa-8-azaspiro[4.5]decan-4-amine (CAS 2240982-57-2), 4˜ [(38,4S)-4-amino-3-methyl-2-oxa-8-azaspiro[4.5]dec-8-yl]-7-(2,3-dichlorophenyl)-6-methyl-pyrazolo[1,5-a]pyrazine-2-methanol (CAS 2240982-69-6), 7-[(2-amino-3-chloro-4-pyridinyl)thio]-4-[(38,4S)-4-amino-3-methyl-2-oxa-8-azaspiro[4.5]dec-8-yl]-6-methyl-pyrazolo[1,5-a]pyrazine-2-methanol (CAS 2240982-73-2), and (38,4S)-8-[7-[(2-amino-3-chloro-4-pyridinyl)thio]-6-methylpyrazolo[1,5-a]pyrazin-4-yl]-3-methyl-2-oxa-8-azaspiro[4.5]decan-4-amine (CAS 2240982-77-6). In some cases, the SHP inhibitor for use in the methods provided herein is (IR)-8-[5-(2,3-dichlorophenyl)-6-methylimidazo[1,5-a]pyrazin-8-yl]-8-azaspiro[4.5]decan-1-amine (CAS 2240981-78-4). In some cases, exemplary SHP2 inhibitors for use in the methods provided herein include, but are not limited to 3˜ [(IR)-1-amino-8-azaspiro[4.5]dec-8-yl]-6-(2,3-dichlorophenyl)-5-hydroxy-2-pyridinemethanol (CAS 2238840-54-3), 3-[(1R)-1-amino-8-azaspiro[4.5]dec-8-yl]-6-[(2,3-dichlorophenyl)thio]-5-hydroxy-2-pyridinemethanol (CAS 2238840-56-5), 5-[(1R)-1-amino-8-azaspiro[4.5]dec-8-yl]-2-(2,3-dichlorophenyl)-3-pyridinol (CAS 2238840-58-7), 3-[(1R)-1-amino-8-azaspiro[4.5]dec-8-yl]-6-(2,3-dichlorophenyl)-5-methyl-2-pyridinemethanol (CAS 2238840-60-1), (1R)-8-[6-(2,3-dichlorophenyl)-5-methyl-3-pyridinyl]-8-azaspiro[4.5]decan-1-amine (CAS 2238840-62-3), 3-[(1R)-1-amino-8-azaspiro[4.5]dec-8-yl]-6-[(2,3-dichlorophenyl)thio]-5-methyl-2-pyridinemethanol (CAS 2238840-63-4), (1R)-8-16-[(2,3-dichlorophenyl)thio]-5-methyl-3-pyridinyl]-8-azaspiro[4.5]decan-1-amine (CAS 2238840-64-5), 5-(4-amino-4-methyl-1-piperidinyl)-2-[(2,3-dichlorophenyl)thio]-3-pyridinol (CAS 2238840-65-6), 5-[(IR)-1-amino-8-azaspiro[4.5]dec-8-yl]-2-[(2,3-dichlorophenyl)thio]-3-pyridinol (CAS 2238840-66-7), 6-[(2-amino-3-chloro-4-pyridinyl)thiol-3-[(38,4S)-4-amino-3-methyl-2-oxa-8-azaspiro[4.5]dec-8-yl]-5-hydroxy-2-pyridinemethanol (CAS 2238840-67-8), 3-(4-amino-4-methyl-1-piperidinyl)-6-(2,3-dichlorophenyl)-5-hydroxy-2-pyridinemethanol (CAS 2238840-68-9), 3-[(3S,4S)-4-amino-3-methyl-2-oxa-8-azaspiro[4.5]dec-8-yl]-6-(2,3-dichlorophenyl)-5-methyl-2-pyridinemethanol (CAS 2238840-69-0), 6-[(2-amino-3-chloro-4-pyridinyl)thio]-3-[(38,4S)-4-amino-3-methyl-2-oxa-8-azaspiro[4.5]dec-8-yl]-5-methyl-2-pyridinemethanol (CAS 2238840-70-3), 3-(4-amino-4-methyl-1-piperidinyl)-6-(2,3-dichlorophenyl)-5-methyl-2-pyridinemethanol (CAS 2238840-71-4), 6-[(2-amino-3-chloro-4-pyridinyl)thio]-3-(4-amino-4-methyl-1-piperidinyl)-2-pyridinemethanol (CAS 2238840-72-5). 5-[(2-amino-3-chloro-4-pyridinyl)thio]-2-[(38,4S)-4-amino-3-methyl-2-oxa-8-azaspiro[4.5]dec-8-yl]-6-methyl-3-pyridinemethanol (CAS 2238840-73-6), 2-[(38,4S)-4-amino-3-methyl-2-oxa-8-azaspiro[4.5]dec-8-yl]-5-(2,3-dichlorophenyl)-6-methyl-3-pyridinemethanol (CAS 2238840-74-7), 3-[(3S,4S)-4-amino-3-methyl-2-oxa-8-azaspiro[4.5]dec-8-yl]-6-(2,3-dichlorophenyl)-5-hydroxy-2-pyridinemethanol (CAS 2238840-75-8), and 2-[(2-amino-3-chloro-4-pyridyl)sulfanyl]-5-[(3S,4S)-4-amino-3-methyl-2-oxa-8-azaspiro[4.5]decan-8-yl]-6-(hydroxymethyl)pyridin-3-ol. In some cases, the SHP inhibitor for use in the methods provided herein is 3-[(1R)-1-amino-8-azaspiro[4.5]dec-8-yl]-6-[(2,3-dichlorophenyl)thio]-5-hydroxy-2-pyridinemethanol (CAS 2238840-56-5). In some cases, the SHP2 inhibitor for use in the methods provided herein is an inhibitor disclosed in U.S. Pat. No. 10,590,090 B2, US 2020/017517 A1, US 2020/017511 A1, WO 2019/075265 A1, or WO 2021/142026, each of which is herewith incorporated by reference in its entirety.

SOSI Inhibitors. In some cases, the compounds of the disclosure can be administered simultaneously, separately, or sequentially with an effective amount of a SOSI inhibitor in any of the methods described herein. Exemplary SOSI inhibitors for use in the methods provided herein include, but are not limited to, BI 3406 (N-[(1R)-1-13-amino-5-(trifluoromethyl)phenyl]ethyl]-7-methoxy-2-methyl-6-[(3S)-oxolan-3-yl]oxyquinazolin-4-amine), BI 1701963, AST-NS2102, MRTX-0902 ((R)-2-methyl-3-(1-((4-methyl-7-morpholinopyrido[3,4-d]pyridazin-]-yl)amino)ethyl)benzonitrile), ERAS-9, RMC-5845, HM-99462, and GH-52.

Src Kinase Inhibitors. In some cases, the compounds of the disclosure can be administered simultaneously, separately, or sequentially with an effective amount of a Sre kinase inhibitor in any of the methods described herein. The term “Src kinase” as used herein refers to a member of a mammalian nonreceptor tyrosine kinase family including: Src, Yes, Fyn, and Fgr (SrcA subfamily); Lck, Hck, Blk, and Lyn (SrcB subfamily), and Frk subfamily. The term “Src kinase inhibitor” as used herein refers to a compound that is capable of negatively modulating or inhibiting all or a portion of the enzymatic activity of one or more member of the Sre kinases. Exemplary Sre kinase inhibitors for use in the methods provided herein include, but are not limited to, dasatinib, ponatinib, vandetanib, bosutinib, saracatinib, KX2-391 (N-benzyl-2-(5-(4-(2-morpholinoethoxy)phenyl)pyridin-2-yl) acetamide), SU6656 ((Z)—N,N-dimethyl-2-oxo-3-((4,5,6,7-tetrahydro-1H-indol-2-yl)methylene) indoline-5-sulfonamide), PP 1 (1-(tert-butyl)-3-(p-tolyl)-1H-pyrazolo[3,4-d]pyrimidin-4-amine), WH-4-023 (2,6-dimethylphenyl(2,4-dimethoxyphenyl) (2-((4-(4-methylpiperazin-1-yl)phenyl)amino)pyrimidin-4-yl) carbamate), and KX-01 (N-benzyl-2-(5-(4-(2-morpholinoethoxy)phenyl)pyridin-2-yl) acetamide). In some cases, the Sre kinase inhibitor is dasatinib. In some cases, the Src kinase inhibitor is saracatinib. In some cases, the Sre kinase inhibitor is ponatinib. In some cases, the Src kinase inhibitor is vandetanib. In some cases, the Sre kinase inhibitor is KX-01.

Chemotherapeutic Agents. In some cases, the compounds of the disclosure can be administered simultaneously, separately, or sequentially with an effective amount of one or more chemotherapeutic agents in any of the methods described herein. Exemplary chemotherapeutic agents for use in the methods provided herein include, but are not limited to, leucovorin calcium (calcium folinate), 5-fluorouracil, irinotecan, oxaliplatin, cisplatin, carboplatin, pemetrexed, docetaxel, paclitaxel, gemcitabine, vinorelbine, chlorambucil, cyclophosphamide, and methotrexate.

### Definitions and General Terminology

The following definitions are provided to assist in understanding the scope of this disclosure.

Unless otherwise indicated, all numbers expressing quantities of ingredients, reaction conditions, and so forth used in the specification or claims are to be understood as being modified in all instances by the term “about.” Accordingly, unless indicated to the contrary, the numerical parameters set forth in the following specification and attached claims are approximations that may vary depending upon the standard deviation found in their respective testing measurements.

As used herein, if any variable occurs more than one time in a chemical formula, its definition on each occurrence is independent of its definition at every other occurrence. If the chemical structure and chemical name conflict, the chemical structure is determinative of the identity of the compound.

### Stereoisomers

The compounds of the present disclosure may contain, for example, double bonds, one or more asymmetric carbon atoms, and bonds with a hindered rotation, and therefore, may exist as stereoisomers, such as double-bond isomers (i.e., geometric isomers (E/Z)), enantiomers, diastereomers, and atropoisomers. Accordingly, the scope of the present disclosure is to be understood to encompass all possible stereoisomers of the illustrated compounds, including the stereoisomerically pure form (for example, geometrically pure, enantiomerically pure, diastereomerically pure, and atropoisomerically pure) and stereoisomeric mixtures (for example, mixtures of geometric isomers, enantiomers, diastereomers, and atropoisomers, or mixture of any of the foregoing) of any chemical structures disclosed herein (in whole or in part), unless the stereochemistry is specifically identified.

If the stereochemistry of a structure or a portion of a structure is not indicated with, for example, bold or dashed lines, the structure or portion of the structure is to be interpreted as encompassing all stereoisomers of the structure. If the stereochemistry of a structure or a portion of a structure is indicated with, for example, bold or dashed lines, the structure or portion of the structure is to be interpreted as encompassing only the stereoisomer indicated, unless otherwise noted. For example,

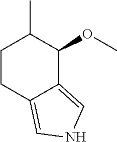

represents

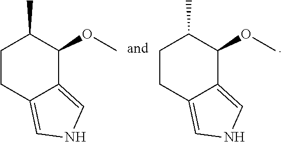

Similarly, for example, the chemical name (4R)-4-methoxy-5-methyl-4,5,6,7-tetrahydro-2H-isoindole represents (4R,5R)-4-methoxy-5-methyl-4,5,6,7-tetrahydro-2H-isoindole and (4R,5S)-4-methoxy-5-methyl-4,5,6,7-tetrahydro-2H-isoindole. A bond drawn with a wavy line may be used to indicate that both stereoisomers are encompassed. This is not to be confused with a wavy line drawn perpendicular to a bond which indicates the point of attachment of a group to the rest of the molecule.

The term “stereoisomer” or “stereoisomerically pure” compound refers to one stereoisomer (for example, geometric isomer, enantiomer, diastereomer and atropoisomer) of a compound that is substantially free of other stereoisomers of that compound. For example, a stereoisomerically pure compound having one chiral center will be substantially free of the mirror image enantiomer of the compound and a stereoisomerically pure compound having two chiral centers will be substantially free of the other enantiomer and diastereomers of the compound. A typical stereoisomerically pure compound comprises greater than about 80% by weight of one stereoisomer of the compound and equal or less than about 20% by weight of other stereoisomers of the compound, greater than about 90% by weight of one stereoisomer of the compound and equal or less than about 10% by weight of the other stereoisomers of the compound, greater than about 95% by weight of one stereoisomer of the compound and equal or less than about 5% by weight of the other stereoisomers of the compound, or greater than about 97% by weight of one stereoisomer of the compound and equal or less than about 3% by weight of the other stereoisomers of the compound.

This disclosure also encompasses the pharmaceutical compositions comprising stereoisomerically pure forms and the use of stereoisomerically pure forms of any compounds disclosed herein. Further, this disclosure also encompasses pharmaceutical compositions comprising mixtures of stereoisomers of any compounds disclosed herein and the use of said pharmaceutical compositions or mixtures of stereoisomers. These stereoisomers or mixtures thereof may be synthesized in accordance with methods well known in the art and methods disclosed herein. Mixtures of stereoisomers may be resolved using standard techniques, such as chiral columns or chiral resolving agents. See, for example, Jacques et al., Enantiomers, Racemates and Resolutions (Wiley-Interscience, New York, 1981); Wilen et al., Tetrahedron 33:2725; Eliel, Stereochemistry of Carbon Compounds (McGraw-Hill, NY, 1962); and Wilen, Tables of Resolving Agents and Optical Resolutions, page 268 (Eliel, Ed., Univ. of Notre Dame Press, Notre Dame, IN, 1972).

### Tautomers

As known by those skilled in the art, certain compounds disclosed herein may exist in one or more tautomeric forms. Because one chemical structure may only be used to represent one tautomeric form, it will be understood that for convenience, referral to a compound of a given structural formula includes other tautomers of said structural formula. For example,

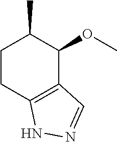

represents

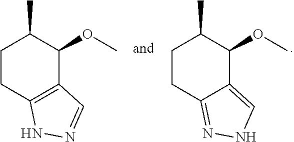

Similarly, for example, the chemical name (4R,5R)-4-methoxy-5-methyl-4,5,6,7-tetrahydro-1H-indazole represents (4R,5R)-4-methoxy-5-methyl-4,5,6,7-tetrabydro-1H-indazole and (4R,5R)-4-methoxy-5-methyl-4,5,6,7-tetrahydro-2H-indazole. Accordingly, the scope of the instant disclosure is to be understood to encompass all tautomeric forms of the compounds disclosed herein.

### Isotopically-Labeled Compounds

Further, the scope of the present disclosure includes all pharmaceutically acceptable isotopically-labelled compounds of the compounds disclosed herein, wherein one or more atoms are replaced by atoms having the same atomic number, but an atomic mass or mass number different from the atomic mass or mass number usually found in nature. Examples of isotopes suitable for inclusion in the compounds disclosed herein include isotopes of hydrogen, such as ²H and ³H, carbon, such as HC, ¹³C and ¹⁴C, chlorine, such as ³Cl, fluorine, such as ¹⁸F, iodine, such as ¹²³I and ¹²⁵I, nitrogen, such as ³³N and ¹⁵N, oxygen, such as ¹⁵O, ¹⁷O and ¹²O, phosphorus, such as ³²P, and sulfur, such as ³⁵S. Certain isotopically-labelled compounds of Formula I, for example, those incorporating a radioactive isotope, are useful in drug and/or substrate tissue distribution studies. The radioactive isotopes tritium (³H) and carbon-14 (¹⁴C) are particularly useful for this purpose in view of their ease of incorporation and ready means of detection. Substitution with isotopes such as deuterium (H or D) may afford certain therapeutic advantages resulting from greater metabolic stability, for example, increased in vivo half-life or reduced dosage requirements, and hence may be advantageous in some circumstances. As such, the term “deuterated” refers to the substitution of one or more hydrogen atoms with one or more deuterium atoms on a particular structure or functional group. Substitution with positron emitting isotopes, such as 1C, 18F, 50 and 1N, can be useful in Positron Emission Topography (PET) studies, for example, for examining target occupancy. Isotopically-labelled compounds of the compounds disclosed herein can generally be prepared by conventional techniques known to those skilled in the art or by processes analogous to those described in the accompanying GENERAL SYNTHETIC PROCEDURES and EXAMPLES sections using an appropriate isotopically-labelled reagent in place of the non-labelled reagent previously employed.

### Definitions

The following definitions are provided to assist in understanding the scope of this disclosure. Unless defined otherwise, all technical and scientific terms used herein have the same meaning as is commonly understood by one of ordinary skill in the art to which this disclosure belongs.

For purposes of this disclosure, the chemical elements are identified in accordance with the Periodic Table of the Elements, CAS version, Handbook of Chemistry and Physics, 75th Ed. Additionally, general principles of organic chemistry are described in Organic Chemistry, Thomas Sorrell, University Science Books, Sausalito: 1999, and March's Advanced Organic Chemistry, 5th Ed., Ed.: Smith, M. B, and March, J., John Wiley & Sons, New York: 2001, the entire contents of which are hereby incorporated by reference.

Unless otherwise indicated, the depictions of partial structures do not represent any particular orientation of the partial structure. For example, compounds of Formula (II) having

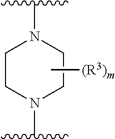

as

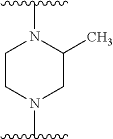 [SMILES: *N1CCN(*)C(C)C1]

includes compounds of Formula (II) depicted

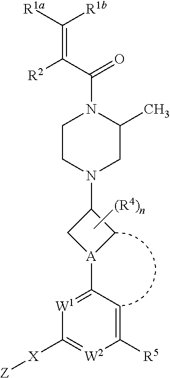

as or

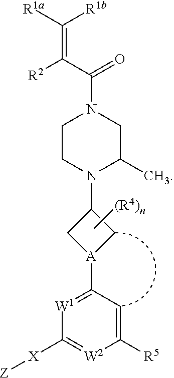

Similarly, compounds of Formula (II) having [symbol] as

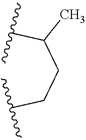 [SMILES: *CCC(*)C]

includes structures wherein

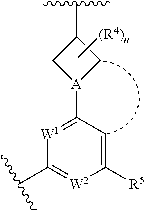

is

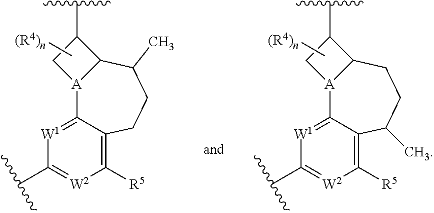

As described herein, compounds described herein may optionally be substituted with one or more substituents, such as illustrated generally below, or as exemplified by particular classes, subclasses, and species described herein. It will be appreciated that the phrase “optionally substituted” is used interchangeably with the phrase “substituted or unsubstituted.” In general, the term “substituted,” whether preceded by the term “optionally” or not, refers to the replacement of one or more hydrogen radicals in a given structure with the radical of a specified substituent. Unless otherwise indicated, an optionally substituted group may have a substituent at each substitutable position of the group. When more than one position in a given structure can be substituted with more than one substituent selected from a specified group, the substituent may be either the same or different at each position. When the term “optionally substituted” precedes a list, said term refers to all of the subsequent substitutable groups in that list. If a substituent radical or structure is not identified or defined as “optionally substituted”, the substituent radical or structure is unsubstituted. Unless otherwise indicated, the substituent is selected from deuterium, halo, oxo, carboxyl, CHO, NH₂, amido, NO₂, ester, thioester, C₀₋₃alkyleneCN, C₁₋₆alkyl, C₁₋₆haloalkyl, C₀₋₆alkylene-OH, C₀₋₆alkylene-C₁₋₄alkoxy, C₀₋₆alkylene-C₁ haloalkoxy, C₀₋₃alkylene-C₁₋₄thioalkoxy, C₀₋₆alkylene-C₁₋₃alkoxy, deuterated C₀₋₃alkylene-OCH-alkoxy, amido, C₀₋₂alkylene-cycloalkyl having 3-7 total ring atoms, C₀₋₂alkylene-cycloalkenyl having 5-7 total ring atoms, C₀₋₂alkylene-heterocycloalkyl having 3-7 total ring atoms and 1-3 heteroatoms selected from N, O, and S, C₀₋₂alkylene-heterocycloalkenyl having 3-7 total ring atoms and 1-3 heteroatoms selected from N, O, and S, and C₀₋₂alkylene-C₆₋₁₀aryl.

Selection of substituents and combinations of substituents contemplated herein are those that result in the formation of stable or chemically feasible compounds. The term “stable”, as used herein, refers to compounds that are not substantially altered when subjected to conditions to allow for their production, detection, and, specifically, their recovery, purification, and use for one or more of the purposes disclosed herein. In some cases, a stable compound or chemically feasible compound is one that is not substantially altered when kept at a temperature of 40° C., or less, in the absence of moisture or other chemically reactive conditions, for at least a week. Only those choices and combinations of substituents that result in a stable structure are contemplated. Such choices and combinations will be apparent to those of ordinary skill in the art and may be determined without undue experimentation.

The term “halo” or “halogen” refers to fluoro (—F), chloro (—Cl), bromo (—Br), or iodo (—I).

The term “oxo” refers to ═O. For example, an oxo substituent on a cyclopentyl ring can be depicted as:

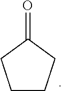

For compounds having multiple occurrences of the same R group on a core structure

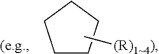

the phrase “wherein two geminal R groups together with the atom to which they are attached form an oxo group” refers in a ═O group attached to a single atom

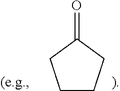

The term “ether” refers to an oxygen atom bonded to two alkyl or aryl groups (R—O—R). The term “ether bridge” refers to an ether group that forms a bridge on a ring, wherein the bridge has the indicated number of carbon atoms. For example, a C₁ ether bridge

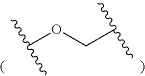 [SMILES: *CO*]

on a cyclohexylene ring cyclohexylene ring can be depicted as, for example,

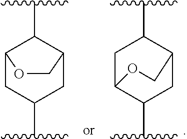 [SMILES: *C1CC2COC(C1)C2* ; *C1CC2OCC1CC2*]

The term “thioether” refers to a sulfur atom bonded to two alkyl or aryl groups (R—S—R). The term “thioether bridge” refers to a thioether group that forms a bridge on a ring, wherein the bridge has the indicated number of carbon atoms. For example, a C₁ thioether bridge

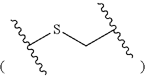 [SMILES: *CS*]

on a cyclohexylene ring cyclohexylene ring can be depicted as, for example,

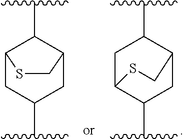 [SMILES: *C1CC2CSC(C1)C2* ; *C1CC2SCC1CC2*]

The term “alkyl” refers to a saturated straight or branched chain hydrocarbon containing the indicated number of carbon atoms. For example, C₃alkyl means the alkyl group has 3 carbon atoms. C₁₋₆alkyl refers to an alkyl group having a number of carbon atoms encompassing the entire range (e.g., 1, 2, 3, 4, 5, or 6 carbon atoms), as well as encompassing all subgroups (e.g., 1-2, 1-3, 1-4, 1-5, 1-6, 2-3, 2-4, 2-5, 2-6, 3-4, 3-5, 3-6, 4-5, 4-6, and 5-6 carbon atoms). Nonlimiting examples of alkyl groups include methyl, ethyl, n-propyl, isopropyl, n-butyl, sec-butyl, isobutyl, tert-butyl, pentyl, and hexyl.

The term “alkenyl” refers to a straight or branched chain hydrocarbon containing the indicated number of carbon atoms and one or more double bonds. For example, C₃alkenyl means the alkenyl group has 3 carbon atoms. C₂₋₆alkenyl refers to an alkenyl group having a number of carbon atoms encompassing the entire range (e.g., 2, 3, 4, 5, or 6 carbon atoms), as well as encompassing all subgroups (e.g., 2-3, 2-4, 2-5, 2-6, 3-4, 3-5, 3-6, 4-5, 4-6, and 5-6 carbon atoms), Nonlimiting examples of alkenyl groups include ethenyl, I-propenyl, 2-propenyl, and butenyl.

The term “alkynyl” refers to a straight or branched chain hydrocarbon containing the indicated number of carbon atoms and one or more triple bonds. For example, C₃alkynyl means the alkynyl group has 3 carbon atoms. C₂₋₆alkynyl refers to an alkynyl group having a number of carbon atoms encompassing the entire range (e.g., 2, 3, 4, 5, and 6 carbon atoms), as well as encompassing all subgroups (e.g., 2-3, 2-4, 2-5, 2-6, 3-4, 3-5, 3-6, 4-5, 4-6, and 5-6 carbon atoms). Nonlimiting examples of alkynyl groups include ethynyl, 1-propynyl, 2-propynyl, and butynyl.

The term “alkylene” refers to a bivalent saturated aliphatic radical containing the indicated number of carbon atoms. For example, C₃alkylene means the alkylene group has 3 carbon atoms. C₁₋₆alkylene refers to an alkylene group having a number of carbon atoms encompassing the entire range (e.g., 1, 2, 3, 4, 5, or 6 carbon atoms), as well as encompassing all subgroups (e.g., 1-2, 1-3, 1-4, 1-5, 1-6, 2-3, 2-4, 2-5, 2-6, 3-4, 3-5, 3-6, 4-5, 4-6, and 5-6 carbon atoms). When the number of carbon atoms in an alkylene group is indicated as “C₀,” then the alkylene group is not present and the recited substituent is directly attached to the rest of the compound. For example, the term C₀₋₆alkylene-OH indicates that the OH group can be directly attached to the compound or through a C₁₋₃alkylene linker.

The term “alkylene bridge” refers to an alkylene group that forms a bridge on a ring, wherein the bridge has the indicated number of carbon atoms. For example, a C₁alkylene bridge

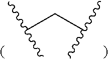 [SMILES: *C*]

on a cyclohexylene ring can be depicted as, for example,

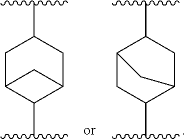 [SMILES: *C1CC2CC(C1)C2* ; *C1CC2CC1CC2*]

A C₂alkylene bridge

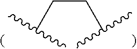 [SMILES: *CC*]

on a cyclohexylene ring can be depicted as, for example,

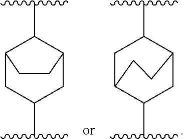 [SMILES: *C1CC2CCC(C1)C2* ; *C1CC2CCC1CC2*]

A C₃alkylene bridge

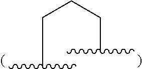 [SMILES: *CCC*]

on a cyclohexylene ring can be depicted as, for example,

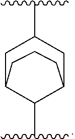 [SMILES: *C1CC2CCCC(C1)C2*]

Additional examples of rings having a C₁₋₂alkylene bridge are

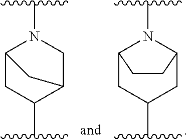 [SMILES: *C1CC2CC1CN2* ; *C1CC2CCC(C1)N2*]

The term “alkenylene” refers to a bivalent straight or branched chain hydrocarbon chain containing the indicated number of carbon atoms and one or more double bonds. For example, C₃alkenylene means the alkenylene group has 3 carbon atoms. C₁₋₆alkenylene refers to an alkenylene group having a number of carbon atoms encompassing the entire range (e.g., 1, 2, 3, 4, 5, or 6 carbon atoms), as well as encompassing all subgroups (e.g., 1-2, 1-3, 1-4, 1-5, 1-6, 2-3, 2-4, 2-5, 2-6, 3-4, 3-5, 3-6, 4-5, 4-6, and 5-6 carbon atoms).

The term “alkenylene bridge” refers to an alkenylene group that forms a bridge on a ring, wherein the bridge has the indicated number of carbon atoms. A C₂alkenylene bridge

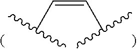 [SMILES: */C=C\*]

on a cyclohexylene ring can be depicted as, for example,

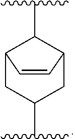 [SMILES: *C1CC2C=CC(C1)C2*]

A C₃alkenylene bridge

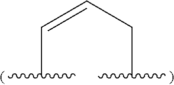 [SMILES: */C=C\C*]

on a cyclohexylene ring can be depicted as, for example,

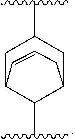 [SMILES: *C1CC2C=CCC(C1)C2*]

The term “heteroatom,” unless otherwise stated herein, refers to an atom that is not carbon or hydrogen. Examples of heteroatoms include oxygen, sulfur, nitrogen, or phosphorus.

The term “haloalkyl” refers to an alkyl group, as previously defined herein, in which one or more of the hydrogen atoms is replaced by a halogen. The halogen is independently selected at each occurrence. The term includes perfluorinated alkyl groups, such as CF₃ and CF₂CF₃. For example, the term “C₁₋₄haloalkyl” refers to a C₁₋₄alkyl as defined herein, wherein one or more hydrogen atoms are substituted with a halogen. Representative examples of C₁₋₄haloalkyl include, but are not limited to, CH₂F, CHF₂,CF₃, CHFCl, CH₂CF₃, CFHCF₃, CF₂CF₃, CH(CF₃)₂, CF(CHF₂)₂, and CH(CH₂F)(CF₃).

The term “heteroalkylene” refers to an alkylene group containing one or more heteroatoms (e.g., one or more of N, O, and S) at one or more of the heteroalkylene's points of attachment (e.g., —OCH₂CH₂O— or —OCH₂CH₂-) or between two carbon atoms (e.g., ether), or a combination thereof. A heteroalkylene contains the indicated number of total atoms (i.e., the sum of the carbon atoms and heteroatoms in the chain). Where a range is indicated, all members of that range and all subgroups within that range are envisioned. For example, a heteroalkylene having 2-6 total atoms and 1, 2, or 3 heteroatoms independently selected from O and S includes heteroalkylene groups having 2, 3, 4, 5, or 6 total atoms in the heteroalkylene chain (or any combination of the foregoing), as well as all subgroups of total atoms in the indicated range (e.g., 2-3, 2-4, 2-5, 2-6, 3-4, 3-5, 3-6, 4-5, 4-6, or 5-6 total atoms, or any combination of the foregoing ranges), wherein 1, 2, or 3 (or any combination of the foregoing) of the total atoms in the chain are heteroatoms, as well as all subgroups in the indicated range (e.g., 1-2, 1-3, or 2-3 heteroatoms, or any combination of the foregoing). Thus, a heteroalkylene having 5-7 total atoms and 1-3 heteroatoms independently selected from N, O, and S encompasses moieties containing, for example, 4 carbon atoms and 1 heteroatom, 3 carbon atoms and 2 heteroatoms, 2 carbon atoms and 3 heteroatoms, 5 carbon atoms and 1 heteroatom. 4 carbon atoms and 2 heteroatoms, 3 carbon atoms and 3 heteroatoms, 6 carbon atoms and 1 heteroatom, 5 carbon atoms and 2 heteroatoms, and 4 carbon atoms and 3 heteroatoms, wherein each heteroatom of the foregoing independently is selected from N, O, and S. Nonlimiting examples of heteroalkylene groups include —O(CH₂)₂O—.

The term “heteroalkenylene” refers to an alkenylene group containing one or more heteroatoms (e.g., one or more of N, O, and S) at one or more of the heteroalkenylene's points of attachment, between two carbon atoms, or a combination thereof. A heteroalkenylene contains the indicated number of total atoms (i.e., the sum of the carbon atoms and heteroatoms in the chain). Where a range is indicated, all members of that range and all subgroups within that range are envisioned. For example, a heteroalkenylene having 4-6 total atoms and 1 or 2 heteroatoms independently selected from O and S includes heteroalkenylene groups having 4, 5, or 6 total atoms in the heteroalkenylene chain (or any combination of the foregoing), as well as all subgroups of total atoms in the indicated range (e.g., 4-5, 4-6, or 5-6 total atoms, or any combination of the foregoing ranges), wherein 1 or 2 of the total atoms in the chain are heteroatoms. Thus, a heteroalkenylene having 5-7 total atoms and 1-3 heteroatoms independently selected from N, O, and S encompasses moieties containing, for example, 4 carbon atoms and 1 heteroatom, 3 carbon atoms and 2 heteroatoms, 2 carbon atoms and 3 heteroatoms, 5 carbon atoms and 1 heteroatom, 4 carbon atoms and 2 heteroatoms, 3 carbon atoms and 3 heteroatoms, 6 carbon atoms and 1 heteroatom, 5 carbon atoms and 2 heteroatoms, and 4 carbon atoms and 3 heteroatoms, wherein each heteroatom of the foregoing independently is selected from N, O, and S.

The term “alkoxy” refers to an alkyl group, as previously defined herein, attached to the molecule through an oxygen atom (e.g., —O-alkyl). Nonlimiting examples of alkyl groups include methoxy, ethoxy, propoxy, iso-propoxy, and butoxy.

The terms “thioalkyl” and “thioalkoxy” are interchangeable and refer to an alkyl group, as previously defined herein, attached to the molecule through a sulfur atom (e.g., —S-alkyl).

The term “haloalkoxy” refers to an alkoxyl group, as previously defined herein, in which one or more of the hydrogen atoms is replaced by a halogen. The term includes perfluorinated alkyl groups, such as OCF₃ and OCF₂CF₃. Representative examples of C₁₋₄haloalkoxy include, but are not limited to, OCH₂F, OCHF₂, OCF₃, OCHFCl, OCH₂CF₃, OCFHCF₃, OCF₂CF₃, OCH(CF₃)₂, OCF(CHF₂)₃, and OCH(CH₂F)(CF₃).

The term “cycloalkyl” refers to an aliphatic cyclic hydrocarbon group containing the indicated number of carbon atoms in its ring. For example, C₅cycloalkyl refers to a cycloalkyl group that has 5 carbon atoms in the ring. C₃₋₇cycloalkyl refers to cycloalkyl group having a number of carbon atoms encompassing the entire range (e.g., 3, 4, 5, 6, and 7 carbon atoms in the ring), as well as encompassing all subgroups (e.g., 3-4, 3-5, 3-6, 3-7, 4-5, 4-6, 4-7, 5-6, 5-7, and 6-7 carbon atoms in the ring). Nonlimiting examples of cycloalkyl groups include cyclopropyl, cyclobutyl, cyclopentyl, and cyclohexyl. The term “spiro-cycloalkyl” refers to a cycloalkyl group as previously defined herein that is attached to the compound through one common atom. For example, a methylpiperidine ring that has a spiro-cyclopropyl group as a substituent can be depicted as:

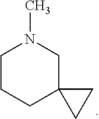

The terms “fused cycloalkyl ring” or “fused-cycloalkyl” can be used interchangeably and refer to a cycloalkyl group, as previously defined herein, that shares two vicinal atoms (i.e., one covalent bond) with the compound to which it is attached. For example, a methylpiperidine ring that has a fused cyclopropyl group as a substituent can be depicted as:

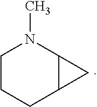

The term “cycloalkenyl” refers to a cyclic hydrocarbon group containing the indicated number of carbon atoms in its ring and one or more double bonds. For example, C₅cycloalkenyl refers to a cycloalkenyl group that has 5 carbon atoms in the ring. C₅₋₇cycloalkenyl refers to cycloalkenyl group having a number of carbon atoms encompassing the entire range (e.g., 5, 6, and 7 carbon atoms in the ring), as well as encompassing all subgroups (e.g., 5-6, 5-7, and 6-7 carbon atoms in the ring). Nonlimiting examples of cycloalkyl groups include cyclopentenyl, and cyclohexenyl.

The term “heterocycloalkyl” refers to a saturated ring comprising carbon and 1, 2, or 3 heteroatoms, and having the indicated number of total ring atoms (the sum of carbon atoms and heteroatoms in the ring). For example, a heterocycloalkyl having 5 total atoms and 2 heteroatoms selected from N and S, refers to a ring having 3 carbon atoms and 2 heteroatoms, wherein each heteroatom of the ring independently is N or S. As another example, a heterocycloalkyl having 5-7 total ring atoms and 1-3 heteroatoms selected from N, O, and S refers to a ring having a total number of ring atoms in the indicated range (e.g., 5, 6, or 7 total atoms), as well as encompassing all subgroups (e.g., 5-6 or 6-7 total ring atoms), wherein 1, 2, or 3 of the atoms in the ring are heteroatoms and each heteroatom is independently selected from N, O, and S. Thus, heterocycloalkyl having 5-7 total ring atoms and 1-3 heteroatoms selected from N, O, and S encompasses rings containing, for example, 4 carbon atoms and 1 heteroatom. 3 carbon atoms and 2 heteroatoms, 2 carbon atoms and 3 heteroatoms, 5 carbon atoms and 1 heteroatom, 4 carbon atoms and 2 heteroatoms, 3 carbon atoms and 3 heteroatoms, 6 carbon atoms and 1 heteroatom, 5 carbon atoms and 2 heteroatoms, and 4 carbon atoms and 3 heteroatoms, wherein each heteroatom of the foregoing is independently selected from N, O, and S. Nonlimiting examples of heterocycloalkyl groups include but are not limited to aziridinyl, azetidinyl, oxetanyl, pyrrolidinyl, pyrazolidinyl, imidazolidinyl, oxazolidinyl, isoxazolidinyl, thiazolidinyl, isothiazolidinyl, tetrahydrofuranyl, tetrahydrothiophenyl, piperidinyl, piperazinyl, tetrahydropyranyl, morpholinyl, azepanyl, diazepanyl, triazepanyl, oxazepanyl, azocanyl, diazocanyl, triazocanyl, oxazocanyl, thiazepanyl, and thiazocanyl. The term “spiro-heterocycloalkyl” refers to a heterocycloalkyl group as previously defined herein that is attached to the compound through one common atom. For example, a methylpiperidine ring that has a spiro-oxetanyl group as a substituent can be depicted as:

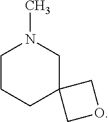

The term “fused-heterocycloalkyl” refers to a heterocycloalkyl group as previously defined herein that shares two vicinal atoms (i.e., one covalent bond) with the compound to which it is attached. For example, a methylpiperidine ring that has a fused-azetidinyl group as a substituent can be depicted as:

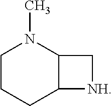

The term “heterocycloalkenyl” is defined similarly to “heterocycloalkyl” except that the ring contains one or more carbon-carbon double bonds.

The term “aryl” refers to an aromatic, carbocylic ring having the indicated number of carbon ring atoms. For example, Caryl refers to an aryl group that has 6 carbon atoms in the ring (e.g., phenyl). Aryl groups can be isolated (e.g., phenyl) or fused to another aryl group (e.g., naphthyl or anthracenyl).

The term “heteroaryl” refers to an aromatic ring comprising carbon and 1, 2, or 3 heteroatoms, and having the indicated number of total ring atoms (the sum of carbon atoms and heteroatoms in the ring). For example, a heteroaryl group having 5 total atoms and 2 heteroatoms selected from N and S, refers to an aromatic ring having 3 carbon atoms and 2 heteroatoms, wherein each heteroatom of the ring independently is N or S. As another example, a heteroaryl having 5-7 total ring atoms and 1-3 heteroatoms selected from N, O, and S refers to an aromatic ring having a total number of ring atoms in the indicated range (e.g., 5, 6, or 7 total atoms), as well as encompassing all subgroups (e.g., 5-6 or 6-7 total ring atoms), wherein 1, 2, or 3 of the atoms in the ring are heteroatoms and each heteroatom is independently selected from N, O, and S. Thus, heteroaryl having 5-7 total ring atoms and 1-3 heteroatoms selected from N, O, and S encompasses rings containing, for example, 4 carbon atoms and 1 heteroatom, 3 carbon atoms and 2 heteroatoms, 2 carbon atoms and 3 heteroatoms, 5 carbon atoms and 1 heteroatom, 4 carbon atoms and 2 heteroatoms, 3 carbon atoms and 3 heteroatoms, 6 carbon atoms and 1 heteroatom, 5 carbon atoms and 2 heteroatoms, and 4 carbon atoms and 3 heteroatoms, wherein each heteroatom of the foregoing is independently selected from N, O, and S. Nonlimiting examples of heteroaryl groups include but are not limited to furanyl, imidazolyl, isothiazolyl, isoxazolyl, oxadiazolyl, oxazolyl, pyrazolyl, pyrrolyl, thiadiazolyl, thiazolyl, thiophenyl, tetrazolyl, triazinyl, triazolyl, pyridyl, pyridazinyl, pyrazinyl, pyrimidinyl, benzofuranyl, benzimidazolyl, benzoisoxazolyl, benzopyranyl, benzothiadiazolyl, benzothiazolyl, benzothienyl, benzothiophenyl, benzotriazolyl, benzoxazolyl, furopyridyl, imidazopyridinyl, imidazothiazolyl, indolizinyl, indolyl, indazolyl, isobenzofuranyl, isobenzothienyl, isoindolyl, isoquinolinyl, isothiazolyl, naphthyridinyl, oxazolopyridinyl, phthalazinyl, pteridinyl, purinyl, pyridopyridyl, pyrrolopyridyl, quinolinyl, quinoxalinyl, quiazolinyl, thiadiazolopyrimidyl, and thienopyridyl.

The term “bicyclic ring” refers a functional group that comprises two joined rings. Unless otherwise indicated, the bicyclic ring may be spirocyclic, in which the two rings share a single atom (e.g., a quaternary carbon atom), fused, in which the two rings share two vicinal atoms (i.e, one covalent bond), or bridged, in which to rings share three or more atoms and contain a bridge having at least one atom.

The terms “protecting group” and “protective group” as used herein, are interchangeable and refer to an agent used to temporarily block one or more desired functional groups in a compound with multiple reactive sites. In some cases, a protecting group has one or more, or specifically all, of the following characteristics: (a) is added selectively to a functional group in good yield to give a protected substrate that is (b) stable to reactions occurring at one or more of the other reactive sites; and (c) is selectively removable in good yield by reagents that do not attack the regenerated, deprotected functional group. As would be understood by one skilled in the art, in some cases, the reagents do not attack other reactive groups in the compound. In other cases, the reagents may also react with other reactive groups in the compound. Examples of protecting groups are detailed in Greene, T. W., Wuts, P. G in “Protective Groups in Organic Synthesis”, Third Edition, John Wiley & Sons, New York: 1999 (and other editions of the book), the entire contents of which are hereby incorporated by reference. The term “nitrogen protecting group”, as used herein, refers to an agent used to temporarily block one or more desired nitrogen reactive sites in a multifunctional compound. In some cases, nitrogen protecting groups also possess the characteristics exemplified for a protecting group above, and certain exemplary nitrogen protecting groups are also detailed in Chapter 7 in Greene, T. W., Wuts. P. G in “Protective Groups in Organic Synthesis”, Third Edition, John Wiley & Sons, New York: 1999, the entire contents of which are hereby incorporated by reference.

The term “bond” indicates that a specified functional group is absent.

The term “geminal” refers to substituents that are attached to the same atom. Geminal R groups on a chain and ring can be depicted as:

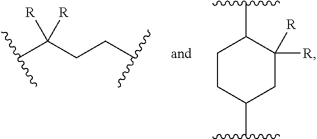

respectively.

The terms “adjacent” and “vicinal” are interchangeable and refer to substituents that are attached to adjacent atoms along a chain or within a ring. Vicinal and adjacent R groups along a chain and within a ring can be depicted as

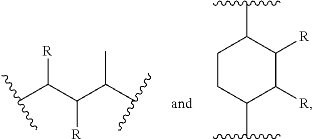

respectively.

The terms “non-neighboring” and “non-adjacent” are interchangeable and refer to substituents that are attached to atoms along a chain or within a ring that are not attached to adjacent atoms and that are not geminal. Non-neighboring R groups along a chain and within a ring can be depicted as

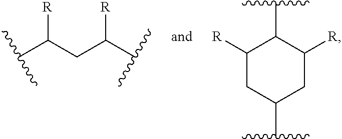

respectively.

The term “pharmaceutically acceptable” as used herein refers to a composition or a component of a composition that is generally safe, non-toxic, and neither biologically nor otherwise undesirable.

The term “pharmaceutically acceptable salt” as used herein refers to a salt of a compound that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, and the like; or (2) salts formed when an acidic proton present in the parent compound either is replaced by a metal ion, for example, an alkali metal ion, an alkaline earth ion, or an aluminum ion; or coordinates with an organic base such as ethanolamine, diethanolamine, triethanolamine, N-methylglucamine, dicyclohexylamine, and the like. Additional examples of such salts can be found in Berge et al., J. Pharm. Sci. 66(1): 1-19 (1977). See also Stahl er al., Pharmaceutical Salts: Properties, Selection, and Use, 2^{nd} Revised Edition (2011).

The term “pharmaceutically acceptable excipient” as used herein refers to a broad range of ingredients that may be combined with a compound or salt disclosed herein to prepare a pharmaceutical composition or formulation. Typically, excipients include, but are not limited to, diluents, colorants, vehicles, anti-adherents, glidants, disintegrants, flavoring agents, coatings, binders, sweeteners, lubricants, sorbents, preservatives, and the like.

The terms “subject” and “patient” as used herein are interchangeable and refer to humans and mammals, including, but not limited to, primates, cows, sheep, goats, horses, dogs, cats, rabbits, rats, and mice. In some cases, the subject is human.

The term “therapeutically effective amount” as used herein refers to that amount of a compound disclosed herein that will elicit the biological or medical response of a tissue, a system, or subject that is being sought by a researcher, veterinarian, medical doctor or other clinician.

The term “metastatic” refers to a cancer that has spread from the place where it first formed to another part of the body. The term non-metastatic refers to a cancer that has not spread from the place where it first formed to another part of the body.

The term “coupled exchange assay” or “2 h coupled exchange assay” as used herein refers to the assay described in the Section entitled “BIOLOGICAL EVALUATION.”

### General Synthetic Procedures

The compounds provided herein can be synthesized according to the procedures described in this and the following sections. The synthetic methods described herein are merely exemplary, and the compounds disclosed herein may also be synthesized by alternate routes utilizing alternative synthetic strategies, as appreciated by persons of ordinary skill in the art. It should be appreciated that the general synthetic procedures and specific examples provided herein are illustrative only and should not be construed as limiting the scope of the present disclosure in any manner.

Generally, the compounds of Formula (II) can be synthesized according to the following schemes. Variables used in the following schemes are the variables as defined for Formula (II), unless otherwise noted. All starting materials are either commercially available, for example, from Merck Sigma-Aldrich Inc., Fluorochem Ltd., and Enamine Ltd, or known in the art and may be synthesized by employing known procedures using ordinary skill. Starting materials may also be synthesized via the procedures disclosed herein. Suitable reaction conditions, such as solvent, reaction temperature, and reagents, for the Schemes discussed in this section, may be found in the examples provided herein. The abbreviation PG refers to a protecting group, as defined herein in the DEFINITIONS AND GENERAL TERMINOLOGY section. In the scheme below, each PG can be the same as or different from another PG in the compound, so long as each protecting group can be selectively removed.

In general, the compounds of Formula (II) can be synthesized according to Scheme 1, below.

### Scheme 1

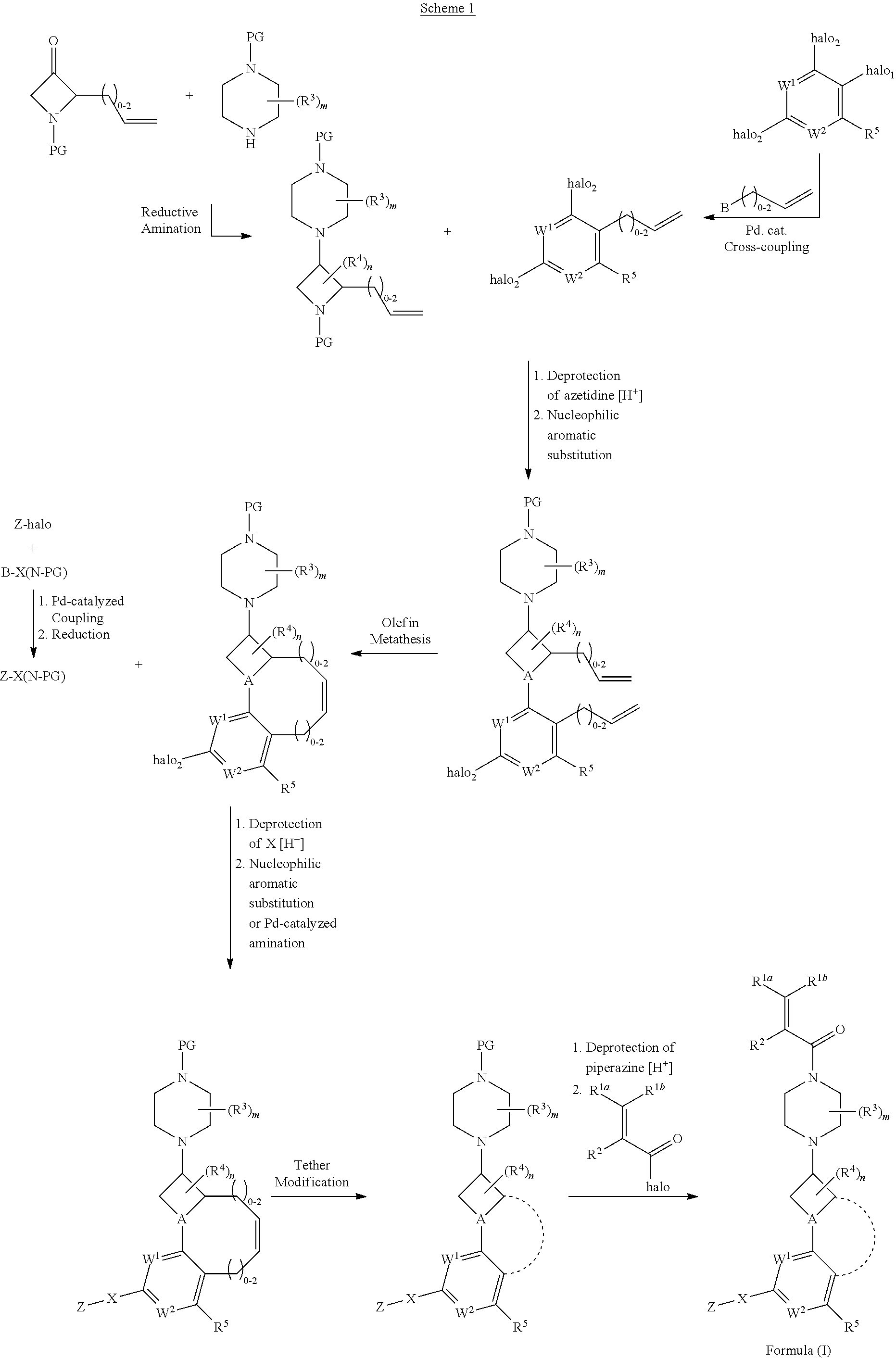

A nitrogen-protected, piperazine linker portion of Formula (II):

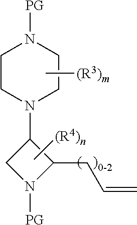

can be synthesized by reacting a desired alkene-substituted, nitrogen-protected, 3-azetidinone with a desired, nitrogen-protected piperazine in the presence of an appropriate reducing reagent, such as a borohydride reagent, in a reductive amination reaction. A desired, alkene-substituted, halogenated aryl/heteroaryl core:

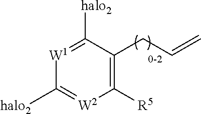

can be synthesized by performing a palladium-catalyzed cross-coupling reaction with

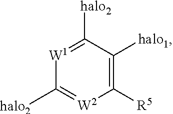

wherein each of halo₁ and halo₂ is a halogen, and a desired, allyl boronic acid compound:

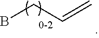

The nitrogen-protected linker portion of Formula (II):

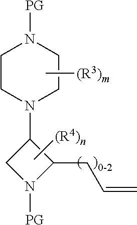

can be coupled to the alkene-substituted, halogenated, aryl/heteroaryl core:

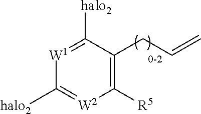

by deprotecting the nitrogen of the azetidine, and conducting a nucleophilic aromatic substitution reaction in the presence of an appropriate base to form a di-alkenyl portion of the compound of Formula (II):

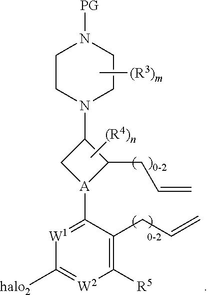

The resulting compound can undergo an olefin metathesis reaction, using for example, Grubb's catalyst, to form the middle portion of Formula (II) having an alkene tether:

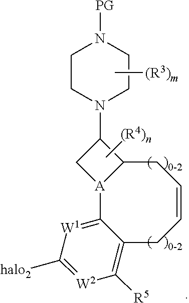

Variable Z can be synthesized by, for example, starting with a desired, optionally substituted, phenyl, heteroaryl, or bicyclic ring, and optionally attaching additional desired substituents to the ring through common techniques known to one skilled in the art. Z-halo can be prepared for coupling by halogenating the phenyl, heteroaryl, or bicyclic ring of Z using, for example, a suitable iodination reagent (e.g., N-iodosuccinimide), bromination reagent (e.g., CBr₄), or chlorination reagent (e.g., (CCl₃)₂), optionally in the presence of a suitable base. The tail portion of Formula (II) can be synthesized by reacting a desired halogenated variable Z (“Z-halo”) with a desired organoboron-functionalized variable X that comprises a protected nitrogen atom (“B-X(N-PG)”) in a palladium-catalyzed coupling reaction to form the Z-X(N-PG) tail portion of Formula (II). When Y of Formula (II) is other than N, then the double bond that results from the coupling reaction can optionally be reduced to a single bond.

The Z-X(N-PG) tail portion of Formula (II) can be coupled to the middle portion of Formula (II) by deprotecting the nitrogen atom of variable X in Z-X(N-PG) to form Z-X(NH), and performing a nucleophilic aromatic substitution with the middle portion of Formula (II) and an appropriate base in a nucleophilic aromatic substitution reaction to form:

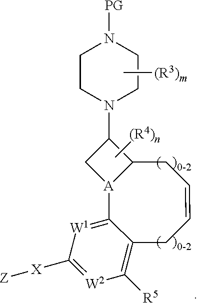

In some cases, the tail portion of Formula (II) can be installed via a palladium-catalyzed amination reaction, such as the Buchwald reaction.

The double bond of the tether can be functionalized to form the compounds of Formula (II). For example, the double bond of the tether can be reduced to a saturated hydrocarbon using a reducing agent, such as Pd/C. Alternatively, the double bond of the tether can be reacted with an allylic oxidizing agent, such as SeO₂, to result in an allylic alcohol. The allylic alcohol can be further oxidized to form an α,β-unsaturated carbonyl (e.g., under Dess-Martin oxidation conditions). The carbon of the α,β-unsaturated carbonyl can undergo difluorination to form an allylic geminal difluoride. The double bond of either the α,β-unsaturated carbonyl or the allylic geminal difluoride can be reduced via a suitable reducing agent to form a tether substituted with a ketone or geminal difluoride, respectively. An alcohol-substituted tether can be formed by subjecting the double bond of the tether to a halogenating agent and an alcohol (e.g., such as N-bromosuccinimide and AcOH) to form a vicinal alkoxyhalide, which can then be epoxidized using a suitable base (e.g., NaOMe), and then reduced (e.g., using Pd/C) to form the alcohol. The alcohol-substituted tether can be oxidized (e.g., using Dess-Martins oxidation conditions) to a ketone, which can then be difluorinated using an organosulfur fluorinating agent, such as diethylaminosulfur trifluoride (DAST).

The Michael acceptor can be installed on the compound by deprotecting the nitrogen atom of the piperazine ring in the presence of an acid, such as TFA, and reacting the deprotected piperazine ring with a desired halogenated α,β-unsaturated ketone, such as acryloyl chloride to form the compound of Formula (II) having an alkene tether.

Compounds of Formula (II) having a tether substituted with a methylene group (═CH₂) can be synthesized similarly to the general procedure described herein for compounds having an alkene tether, except that the tether can be formed via a palladium catalyzed cross-coupling of the nitrogen-protected linker portion of Formula (II):

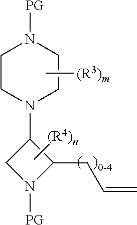

with the aryl halide of the core halogenated, aryl/heteroaryl core:

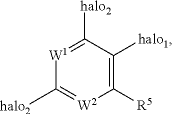

as shown in Scheme 2, below.

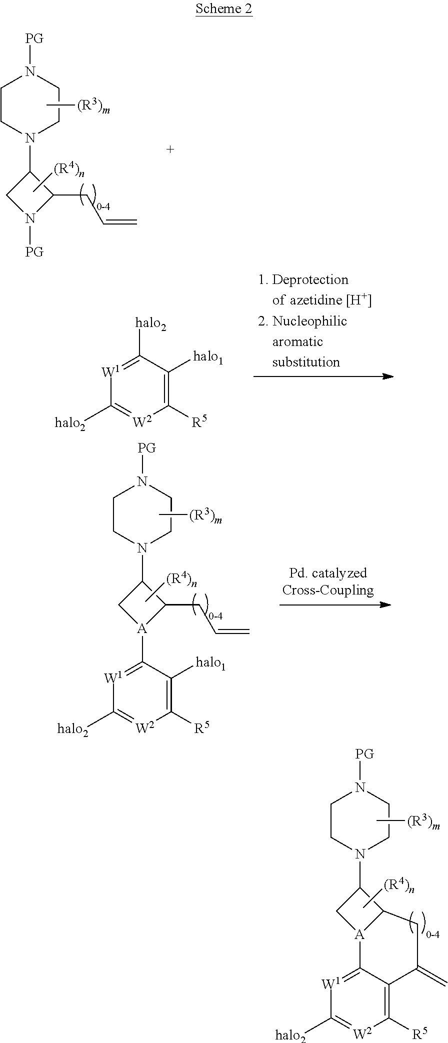

Compounds of Formula (II) having an ether tether can be synthesized similarly to the general procedure described herein for compounds having an alkene tether, except that the tether can be formed by installing an alkylene-OMe group on the azetidine of the nitrogen-protected, linker portion of Formula (II), and coupling the resulting intermediate to a desired core:

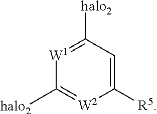

The alkylene can then be demethylated, coupled to chloroacetic acid, and cyclized to the core to form an ether linker, as shown in Scheme 3.

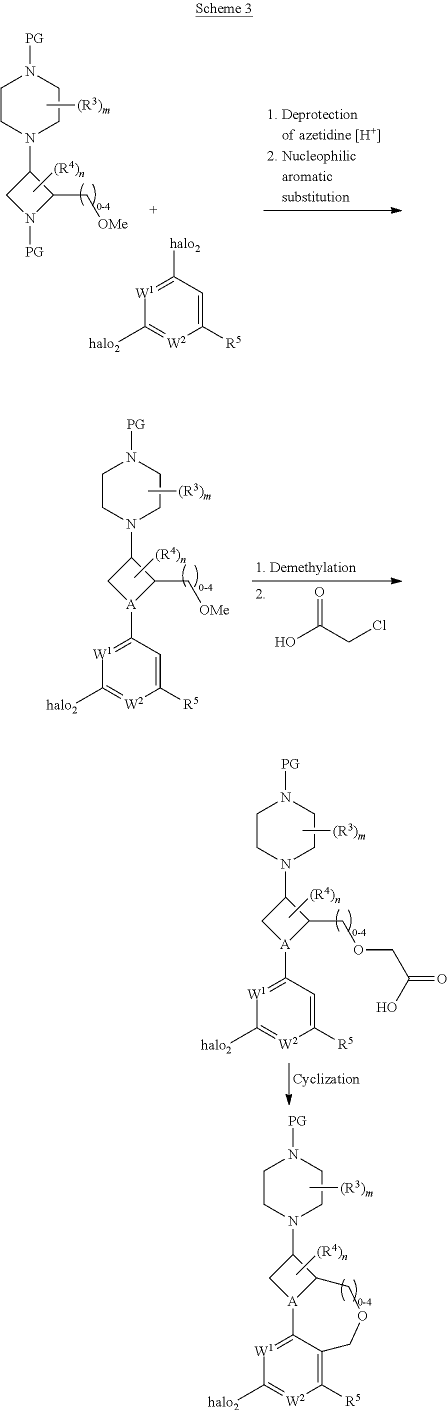

Compounds of Formula (I) can be synthesized similarly to the general procedures described herein for Formula (II).

As can be appreciated by the skilled artisan, the above synthetic scheme and representative examples are not intended to comprise a comprehensive list of all means by which the compounds described and claimed in this application may be synthesized. Further methods will be evident to those of ordinary skill in the art. Additionally, the various synthetic steps described above may be performed in an alternate sequence or order to give the desired compounds.

Purification methods for the compounds described herein are known in the art and include, for example, crystallization, chromatography (for example, liquid, gas phase, and supercritical fluid), extraction, distillation, trituration, and reverse phase HPLC.

### Intermediates

The disclosure further encompasses intermediate compounds, including structures produced from the synthetic procedures described, whether isolated or generated in-situ and not isolated, prior to obtaining the finally desired compound. These intermediates are included in the scope of this disclosure.

Provided herein are intermediates of Formula (Int-AA):

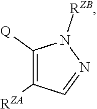

### Formula (Int-AB):

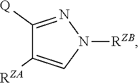

### Formula (Int-AC):

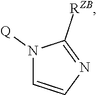

### Formula (Int-AD):

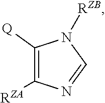

### Formula (Int-AE):

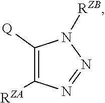

### Formula (Int-AF):

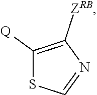

### Formula (Int-AG):

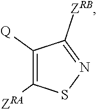

### Formula (Int-AH):

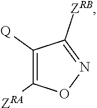

### Formula (Int-AI):

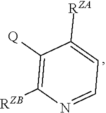

### and Formula (Int-AJ):

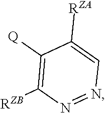

and pharmaceutically acceptable salts of the foregoing; wherein Q is F, Cl, Br, I, or an organoborane (e.g., a pinacolborane) and each of R^{ZA} and R^{ZB} independently is as defined herein for the of the heteroaryl 1 group of Z in the COMPOUNDS OF FORMULA (II) section. In some cases, provided herein are intermediates of Formula (Int-AA):

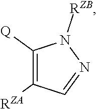

### Formula (Int-AI):

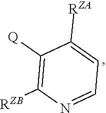

### and Formula (Int-AJ):

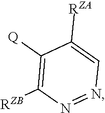

and pharmaceutically acceptable salts of the foregoing. In some cases, provided herein is an intermediate of Formula (Int-AA):

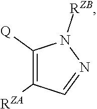

or a pharmaceutically acceptable salt thereof. In some cases, provided herein is an intermediate of Formula (Int-AF):

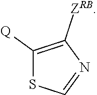

or a pharmaceutically acceptable salt thereof. In some cases, provided herein is an intermediate of Formula (Int-AI):

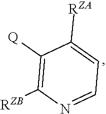

or a pharmaceutically acceptable salt thereof. In some cases, provided herein is an intermediate of Formula (Int-AJ):

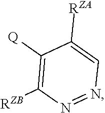

or a pharmaceutically acceptable salt thereof. In some cases, each of R^{ZA} and R^{ZB} independently is halo, CN, C₁₋₃alkyl, C₁₋₆haloalkyl, C₂₋₆alkenyl, C₂₋₆haloalkenyl, C₀₋₃alkylene-OH, C₀₋₃alkylene-C₁₋₃alkoxy, C₀₋₆alkylene-N(R^{N1})₂, C₀₋₂alkylene-C₃₋₆cycloalkyl, C₀₋₂alkylene-heterocycloalkyl having 3-6 total ring atoms and 1-3 heteroatoms selected from N, O, and S, or C₀₋₂alkylene-phenyl; wherein each of the C₃₋₆alkyl, C₂₋₆alkenyl, C₀₋₆alkylene-C₁₋₃alkoxy, cycloalkyl, heterocycloalkyl, and phenyl substituents independently is unsubstituted or substituted with 1-3 further substituents, and each further substituent independently is D, halo, C₁₋₃alkyl, C₁₋₃haloalkyl, C₁₋₂alkyleneOH, C₁₋₂alkylene-C₁₋₃alkoxy, C₁₋₃deuterated alkoxy, N(R^{N1})₂, (C═O) C₁₋₃alkyl, C₅₋₆cycloalkyl, heterocycloalkyl having 3-5 total ring atoms and 1 or 2 heteroatoms selected from N, O, and S; or two geminal further substituents, together with the atom to which they are attached, form spiro-C₃₋₅cycloalkyl, or spiro-heterocycloalkyl having 3-5 total ring atoms and 1 or 2 heteroatoms selected from N, O, and S; or two vicinal further substituents, together with the atoms to which they are attached, form fused-C₃₋₅cycloalkyl or fused-heterocycloalkyl having 3-5 total ring atoms and 1 or 2 heteroatoms selected from N, O, and S; wherein each of the foregoing cycloalkyl and heterocycloalkyl further substituents independently is unsubstituted or substituted with 1 or 2, substituents, and each substituent independently is halo or C₁₋₃alkyl. In some cases, R^{ZA} is CH₃; and R^{ZB} is CH₃, C(CH₃)₂CH₂OH, CH₂CH₂OCH₃, CH₂CH₂OCD₃, CH(CH₃)OCH₃,

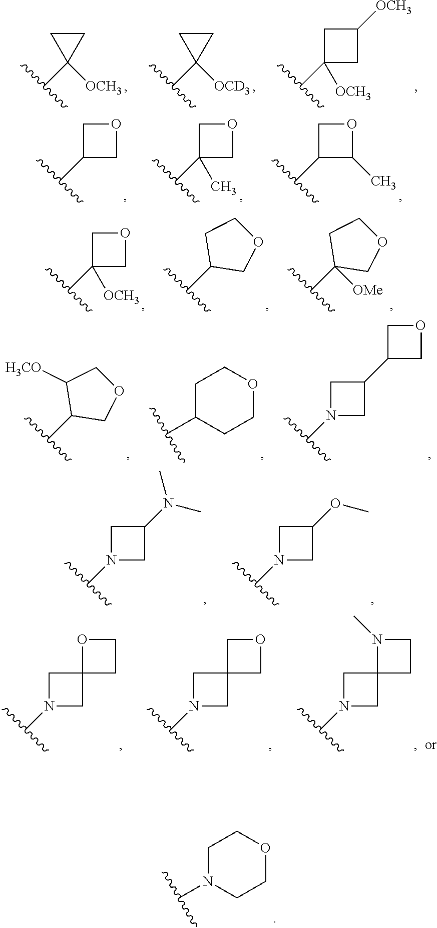

In some cases, R^{ZA} is CH₃; and R^{ZB} is CH₃, CH₂CH₂OCH₃,

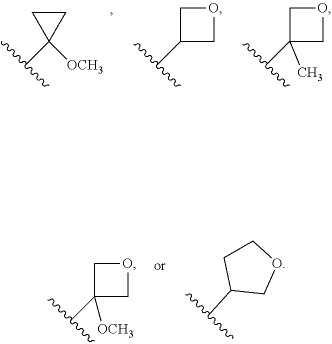

Contemplated examples of intermediates of Formulae Int-AA to Int-AJ are listed in Table INT-A, and pharmaceutically acceptable salts thereof.

TABLE INT-A

| Intermediate | | |
|---|---|---|
| Ref. | Structure | Name |
| A1 | 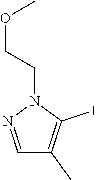 | 5-iodo-1-(2-methoxyethyl)-4-methyl-1H- pyrazole |
| A2 | 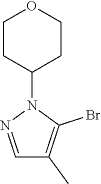 | 5-bromo-4-methyl-1-(tetrahydro-2H- pyran-4-yl)-1H-pyrazole |
| A3 | 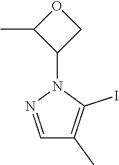 | 5-iodo-4-methyl-1-(2-methyloxetan-3-yl)- 1H-pyrazole |
| A4 | 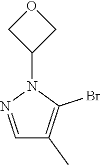 | 5-bromo-4-methyl-1-(oxetan-3-yl)- 1H-pyrazole |
| A5 | 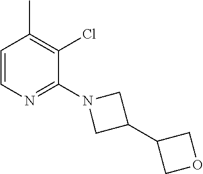 [SMILES: Cc1ccnc(N2CC(C3COC3)C2)c1Cl] | 3-chloro-4-methyl-2-(3-(oxetan-3- yl)azetidin-1-yl)pyridine |
| A6 | 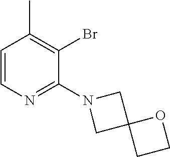 | 6-(3-bromo-4-methylpyridin-2-yl)-1-oxa- 6-azaspiro[3.3]heptane |
| A7 | 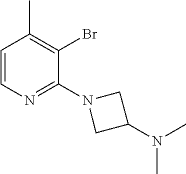 | 1-(3-bromo-4-methylpyridin-2-yl)-N,N- dimethylazetidin-3-amine |
| A8 | 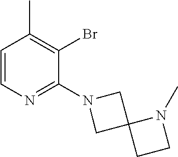 [SMILES: Cc1ccnc(N2CC3(CCN3C)C2)c1Br] | 6-(3-bromo-4-methylpyridin-2-yl)-1- methyl-1,6-diazaspiro[3.3]heptane |
| A9 | 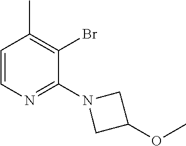 | 3-bromo-2-(3-methoxyazetidin-1-yl)-4- methylpyridine |
| A10 | 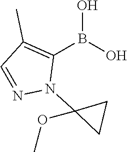 | (1-(1-methoxycyclopropyl)-4-methyl-1H- pyrazol-5-yl)boronic acid |
| A11 | 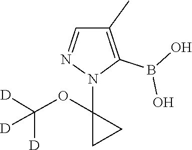 [SMILES: [2H]C([2H])([2H])OC1(n2ncc(C)c2B(O)O)CC1] | (1-(1-(methoxy-d₃)cyclopropyl)-4- methyl-1H-pyrazol-5-yl)boronic acid |
| A12 | 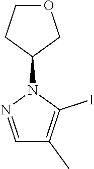 | (S)-5-iodo-4-methyl-1-(tetrahydrofuran-3- yl)-1H-pyrazole |
| A12-1 | 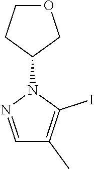 | (R)-5-iodo-4-methyl-1-(tetrahydrofuran- 3-yl)-1H-pyrazole |
| A13 | 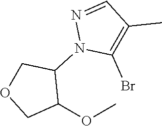 | 5-bromo-4-methyl-1-(3-methyloxetan-3- yl)-1H-pyrazole |
| A14 | 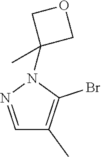 | 5-bromo-4-methyl-1-(3-methyloxetan-3- yl)-1H-pyrazole |
| A15 | 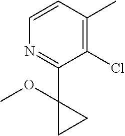 | 3-chloro-2-(1-methoxycyclopropyl)-4- methylpyridine |
| A16 | 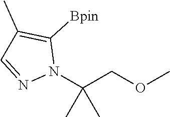 | 1-(1-methoxy-2-methylpropan-2-yl)-4- methyl-5-(4,4,5,5-tetramethyl-1,3,2- dioxaborolan-2-yl)-1H-pyrazole |
| A17 | 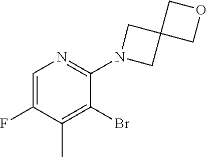 [SMILES: Cc1c(F)cnc(N2CC3(COC3)C2)c1Br] | 6-(3-bromo-5-fluoro-4-methylpyridin-2- yl)-2-oxa-6-azaspiro[3.3]heptane |
| A18 | 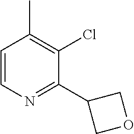 | 3-chloro-4-methyl-2-(oxetan-3- yl)pyridine |

Additional contemplated examples of intermediates are listed in Table Int-A′, below, and pharmaceutically acceptable salts thereof.

TABLE INT-A′

| Intermediate | | |
|---|---|---|
| Ref. | Structure | Name |
| A19 | 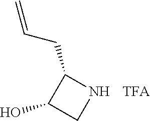 | (2S,3S)-2-allylazetidin-3-ol |
| A19-1 | 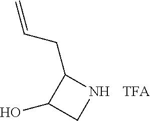 | 2-allylazetidin-3-ol |
| A20 | 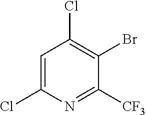 [SMILES: FC(F)(F)c1nc(Cl)cc(Cl)c1Br] | 3-bromo-4,6-dichloro-2- (trifluoromethyl)pyridine |
| A21 | 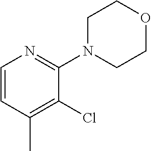 | 4-(3-chloro-4-methylpyridin-2- yl)morpholine |

Further provided herein are intermediates of Formula (Int-B):

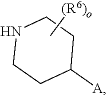

nitrogen-protected analogs thereof (e.g., a BOC-protected analog, such as

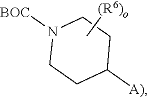

and pharmaceutically acceptable salts of any of the foregoing, wherein A is

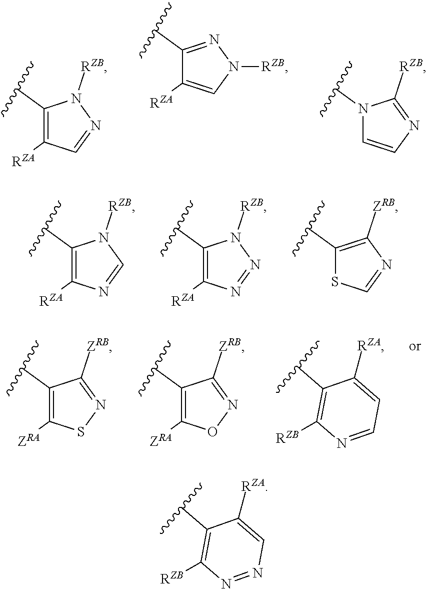

wherein each of o and R⁶ is as defined in the COMPOUNDS OF FORMULA (II) section, and each R^{ZA}, and R^{ZB} is as defined herein in the COMPOUNDS OF FORMULA (II) section and for Intermediates of Formula (Int-AA), Formula (Int-AB), Formula (Int-AC), Formula (Int-AD), Formula (Int-AE), Formula (Int-AF), Formula (Int-AG), Formula (Int-AH), Formula (Int-AI), and Formula (Int-AJ). In some cases, o is 0, 1, 2, 3, or 4; each R⁶ independently is Br, Cl, F, CN, CH₃, CH₂F, CHF₂, CF₃, OH, CH₂OH, OCH₃, OCD₃, CH₂OCH₃, or CH₂N(CH₃)₂, or two geminal R⁶, together with the atom to which they are attached, form oxo, ═CH₃, spiro-cyclopropyl, spiro-cyclobutyl, spiro-oxetanyl, or spiro-tetrahydrofuranyl, or two vicinal R⁶, together with the atoms to which they are attached, form fused-cyclopropyl, fused-cyclobutyl, or fused-cyclopentyl, and any of the foregoing spiro and fused rings is unsubstituted or substituted with 1 or 2 substituents, and each substituent independently is halo, C₃₋₃alkyl, C₁₋₃haloalkyl, C₀₋₂alkyleneOH, C₀₋₂alkyleneC₁₋₃alkoxy, or C₀₋₂alkyleneCN. For example, Formula (Int-B) includes intermediates of Formula (Int-BA):

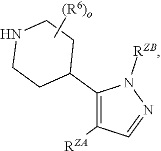

### Formula (Int-BB):

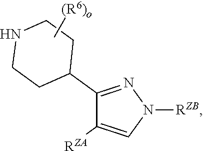

### Formula (Int-BC)

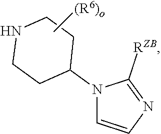

### Formula (Int-BD):

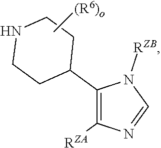

### Formula (Int-BE):

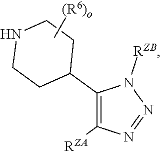

### Formula (Int-BF):

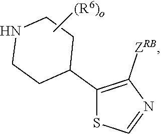

### Formula (Int-BG):

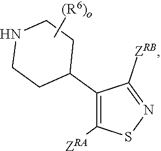

### Formula (Int-BH):

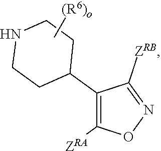

### Formula (Int-BI):

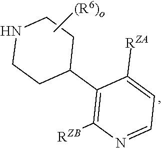

### Formula (Int-BJ):

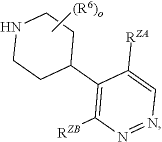

### Formula (Int-BK):

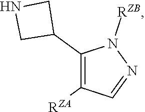

### Formula (Int-BL):

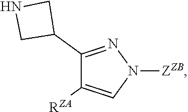

### Formula (Int-BM):

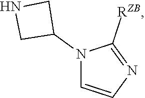

### Formula (Int-BN):

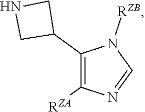

### Formula (Int-BO):

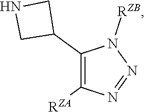

### Formula (Int-BP):

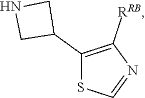

### Formula (Int-BQ):

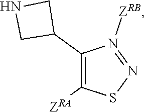

### Formula (Int-BR):

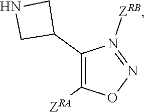

### Formula (Int-BS):

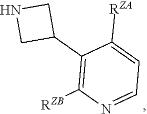

### and Formula (Int-BT):

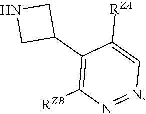

nitrogen-protected analogs of any of the foregoing (e.g., N-BOC protected analogs, such as

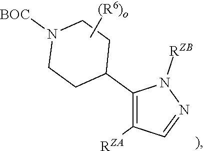

and pharmaceutically acceptable salts of any of the foregoing. In some cases, the disclosure provides an intermediate of

### Formula (INT-BA):

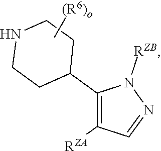

### Formula (INT-BF):

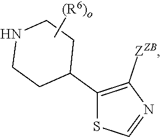

### Formula (INT-BI):

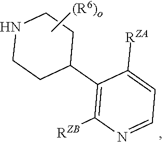

### or Formula (INT-BJ):

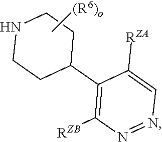

a nitrogen-protected analog any of the foregoing, or a pharmaceutically acceptable salt of any of the foregoing. In some cases, the disclosure provides an intermediate of Formula (Int-BA):

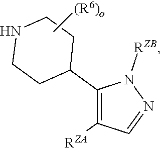

a nitrogen-protected analog thereof, or a pharmaceutically acceptable salt of any of the foregoing. In some cases, the disclosure provides an intermediate of Formula (Int-BF):

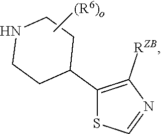

a nitrogen-protected analog thereof, or a pharmaceutically acceptable salt of any of the foregoing. In some cases, the disclosure provides an intermediate of Formula (Int-BI):

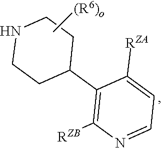

a nitrogen-protected analog thereof, or a pharmaceutically acceptable salt of any of the foregoing. In some cases, the disclosure provides an intermediate of Formula (Int-BJ):

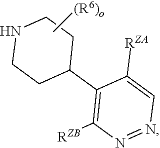

a nitrogen-protected analog thereof, or a pharmaceutically acceptable salt of any of the foregoing. In some cases, o is 0 or 1; R⁶ is CH₃; R^{ZA} is CH₃; and R^{ZB} is CH₃, C(CH₃)₂CH₂OH, CH₂CH₂OCH₃, CH₂CH₂OCD₃, CH(CH₃)OCH₃,

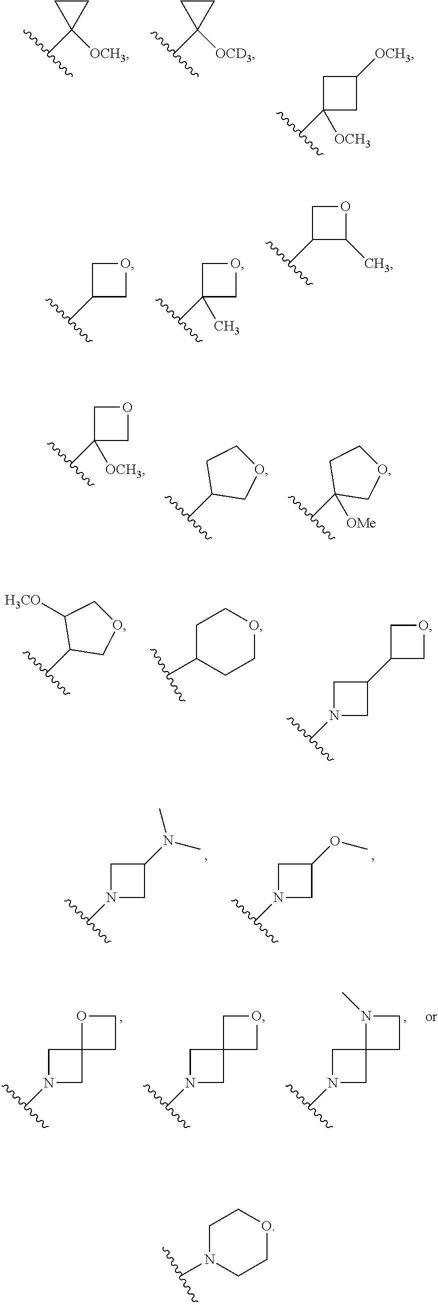

In some cases, R^{ZA} is CH₃; and R^{ZB} is CH₃, CH₂CH₂OCH₃,

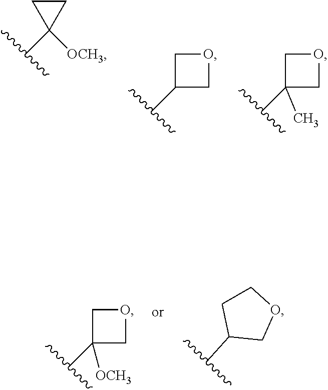

Contemplated examples of intermediates of Formula (Int-B), such as Formulae Int-BA to Int-BT, are listed in Table INT-B, and include pharmaceutically acceptable salts thereof.

TABLE INT-B

| Intermediate | | |
|---|---|---|
| Ref. | Structure | Name |
| B1 | 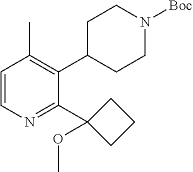 [SMILES: COC1(c2nccc(C)c2C2CCN(C(=O)OC(C)(C)C)CC2)CCC1] | tert-butyl 4-(2-(3-Methoxyoxetan-3-yl)-4- methyl-3-(piperidin-4-yl)pyridine |
| B1-1 | 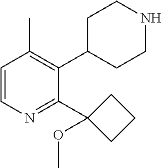 [SMILES: COC1(c2nccc(C)c2C2CCNCC2)CCC1] | 2-(3-methoxyoxetan-3-yl)-4-methyl-3- (piperidin-4-yl)pyridine |
| B2 | 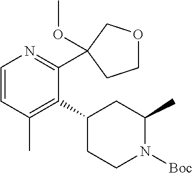 [SMILES: COC1(c2nccc(C)c2[C@H]2CCN(C(=O)OC(C)(C)C)[C@H](C)C2)CCOC1] | tert-butyl (2R,4S)-4-(2-(-3- methoxytetrahydrofuran-3-yl)-4- methylpyridin-3-yl)-2-methylpiperidine- 1-carboxylate |
| B2′ | 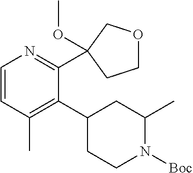 [SMILES: COC1(c2nccc(C)c2C2CCN(C(=O)OC(C)(C)C)C(C)C2)CCOC1] | tert-butyl 4-(2-(3- methoxytetrahydrofuran-3-yl)-4- methylpyridin-3-yl)-2-methylpiperidine- 1-carboxylate |
| B2-1 | 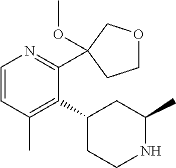 [SMILES: COC1(c2nccc(C)c2[C@H]2CCN[C@H](C)C2)CCOC1] | 2-(3-methoxytetrahydrofuran-3-yl)-4- methyl-3-((2R,4S)-2-methylpiperidin-4- yl)pyridine |
| B2-1′ | 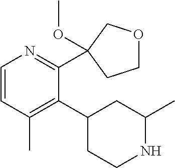 [SMILES: COC1(c2nccc(C)c2C2CCNC(C)C2)CCOC1] | 2-(3-methoxytetrahydrofuran-3-yl)-4- methyl-3-(2-methylpiperidin-4- yl)pyridine |
| B3 | 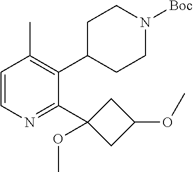 [SMILES: COC1CC(OC)(c2nccc(C)c2C2CCN(C(=O)OC(C)(C)C)CC2)C1] | tert-butyl 4-(2-(1,3- dimethoxycyclobutyl)-4-methylpyridin-3- yl)piperidine-1-carboxylate |
| B3-1 | 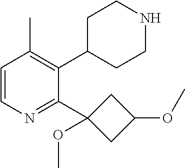 [SMILES: COC1CC(OC)(c2nccc(C)c2C2CCNCC2)C1] | 2-(1,3-dimethoxycyclobutyl)-4-methyl-3- (piperidin-4-yl)pyridine |
| B4 | 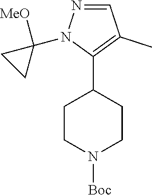 [SMILES: COC1(n2ncc(C)c2C2CCN(C(=O)OC(C)(C)C)CC2)CC1] | tert-butyl 4-(1-(1-methoxycyclopropyl)-4- methyl-1H-pyrazol-5-yl)piperidine-1- carboxylate |
| B4-1 | 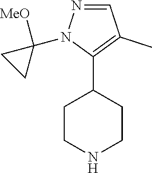 [SMILES: COC1(n2ncc(C)c2C2CCNCC2)CC1] | 4-(1-(1-methoxycyclopropyl)-4-methyl- 1H-pyrazol-5-yl)piperidine |
| B5 | 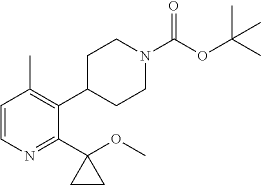 [SMILES: COC1(c2nccc(C)c2C2CCN(C(=O)OC(C)(C)C)CC2)CC1] | tert-butyl 4-(2-(1-methoxycyclopropyl)-4- methylpyridin-3-yl)piperidine-1- carboxylate |
| B5-1 | 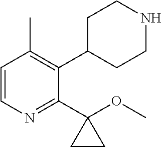 [SMILES: COC1(c2nccc(C)c2C2CCNCC2)CC1] | 2-(1-methoxycyclopropyl)-4-methyl-3- (piperidin-4-yl)pyridine |
| B6 | 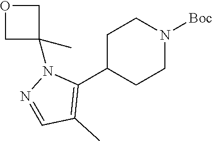 [SMILES: Cc1cnn(C2(C)COC2)c1C1CCN(C(=O)OC(C)(C)C)CC1] | tert-butyl 4-(4-methyl-1-(3-methyloxetan- 3-yl)-1H-pyrazol-5-yl)piperidine-1- carboxylate |
| B6-1 | 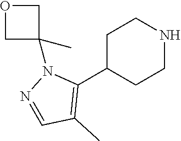 [SMILES: Cc1cnn(C2(C)COC2)c1C1CCNCC1] | 4-(4-methyl-1-(3-methyloxetan-3-yl)-1H- pyrazol-5-yl)piperidine |
| B7 | 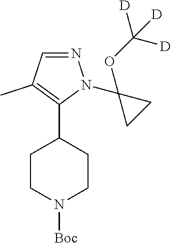 [SMILES: [2H]C([2H])([2H])OC1(n2ncc(C)c2C2CCN(C(=O)OC(C)(C)C)CC2)CC1] | tert-butyl 4-(1-(1-(methoxy- d3)cyclopropyl)-4-methyl-1H-pyrazol-5- yl)piperidine-1-carboxylate |
| B7-1 | 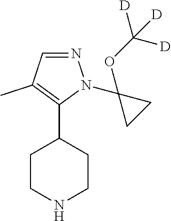 [SMILES: [2H]C([2H])([2H])OC1(n2ncc(C)c2C2CCNCC2)CC1] | 4-(1-(1-(methoxy-d3)cyclopropyl)-4- methyl-1H-pyrazol-5-yl)piperidine |
| B8 | 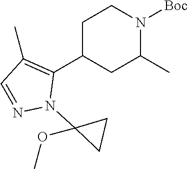 [SMILES: COC1(n2ncc(C)c2C2CCN(C(=O)OC(C)(C)C)C(C)C2)CC1] | tert-butyl (2R,4S)-4-(1-(1- methoxycyclopropyl)-4-methyl-1H- pyrazol-5-yl)-2-methylpiperidine-1- carboxylate |
| B8′ | 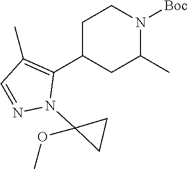 [SMILES: COC1(n2ncc(C)c2C2CCN(C(=O)OC(C)(C)C)C(C)C2)CC1] | tert-butyl 4-(1-(1-methoxycyclopropyl)-4- methyl-1H-pyrazol-5-yl)-2- methylpiperidine-1-carboxylate |
| B8-1 | 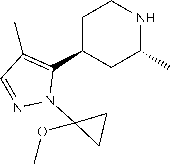 [SMILES: COC1(n2ncc(C)c2[C@H]2CCN[C@H](C)C2)CC1] | (2R,4S)-4-(1-(1-methoxycyclopropyl)-4- methyl-1H-pyrazol-5-yl)-2- methylpiperidine |
| B8-1′ | 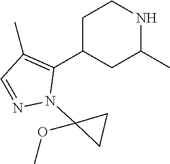 [SMILES: COC1(n2ncc(C)c2C2CCNC(C)C2)CC1] | 4-(1-(1-methoxycyclopropyl)-4-methyl- 1H-pyrazol-5-yl)-2-methylpiperidine |
| B9 | 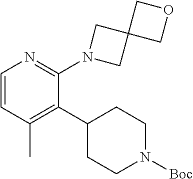 [SMILES: Cc1ccnc(N2CC3(COC3)C2)c1C1CCN(C(=O)OC(C)(C)C)CC1] | tert-butyl 4-(4-methyl-2-(2-oxa-6- azaspiro[3.3]heptan-6-yl)pyridin-3- yl)piperidine-1-carboxylate |
| B9-1 | 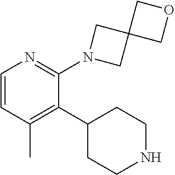 [SMILES: Cc1ccnc(N2CC3(COC3)C2)c1C1CCNCC1] | 6-(4-methyl-3-(piperidin-4-yl)pyridin-2- yl)-2-oxa-6-azaspiro[3.3]heptane |
| B10 | 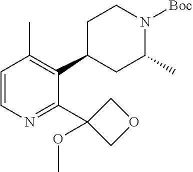 [SMILES: COC1(c2nccc(C)c2[C@H]2CCN(C(=O)OC(C)(C)C)[C@H](C)C2)COC1] | tert-butyl (2R,4S)-4-(2-(3- methoxyoxetan-3-yl)-4-methylpyridin-3- yl)-2-methylpiperidine-1-carboxylate |
| B10′ | 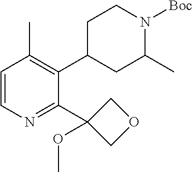 [SMILES: COC1(c2nccc(C)c2C2CCN(C(=O)OC(C)(C)C)C(C)C2)COC1] | tert-butyl 4-(2-(3-methoxyoxetan-3-yl)-4- methylpyridin-3-yl)-2-methylpiperidine- 1-carboxylate |
| B10-1 | 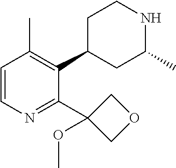 [SMILES: COC1(c2nccc(C)c2[C@H]2CCN[C@H](C)C2)COC1] | 2-(3-methoxyoxetan-3-yl)-4-methyl-3- ((2R,4S)-2-methylpiperidin-4-yl)pyridine |
| B10-1′ | 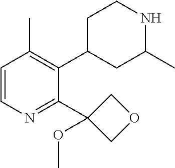 [SMILES: COC1(c2nccc(C)c2C2CCNC(C)C2)COC1] | 2-(3-methoxyoxetan-3-yl)-4-methyl-3-(2- methylpiperidin-4-yl)pyridine |
| B11 | 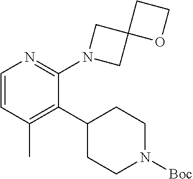 [SMILES: Cc1ccnc(N2CC3(CCO3)C2)c1C1CCN(C(=O)OC(C)(C)C)CC1] | tert-butyl 4-(4-methyl-2-(1-oxa-6- azaspiro[3.3]heptan-6-yl)pyridin-3- yl)piperidine-1-carboxylate |
| B11-1 | 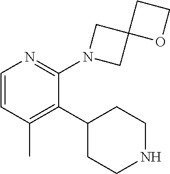 [SMILES: Cc1ccnc(N2CC3(CCO3)C2)c1C1CCNCC1] | 6-(4-methyl-3-(piperidin-4-yl)pyridin-2- yl)-1-oxa-6-azaspiro[3.3]heptane |
| B12 | 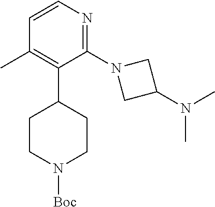 | tert-butyl 4-(2-(3- (dimethylamino)azetidin-1-yl)-4- methylpyridin-3-yl)piperidine-1- carboxylate |
| B12-1 | 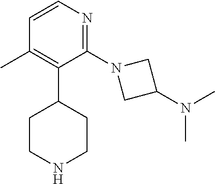 | N,N-dimethyl-1-(4-methyl-3-(piperidin-4- yl)pyridin-2-yl)azetidin-3-amine |
| B13 | 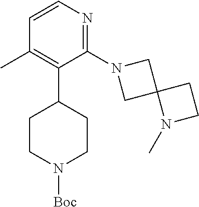 [SMILES: Cc1ccnc(N2CC3(CCN3C)C2)c1C1CCN(C(=O)OC(C)(C)C)CC1] | tert-butyl 4-(4-methyl-2-(1-methyl-1,6- diazaspiro[3.3]heptan-6-yl)pyridin-3- yl)piperidine-1-carboxylate |
| B13-1 | 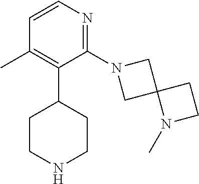 [SMILES: Cc1ccnc(N2CC3(CCN3C)C2)c1C1CCNCC1] | 1-methyl-6-(4-methyl-3-(piperidin-4- yl)pyridin-2-yl)-1,6- diazaspiro[3.3]heptane |
| B14 | 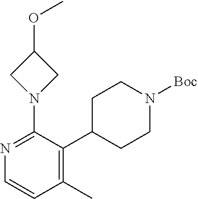 [SMILES: COC1CN(c2nccc(C)c2C2CCN(C(=O)OC(C)(C)C)CC2)C1] | tert-butyl 4-(2-(3-methoxyazetidin-1-yl)- 4-methylpyridin-3-yl)piperidine-1- carboxylate |
| B14-1 | 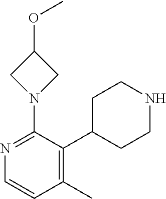 [SMILES: COC1CN(c2nccc(C)c2C2CCNCC2)C1] | 2-(3-methoxyazetidin-1-yl)-4-methyl-3- (piperidin-4-yl)pyridine |
| B15 | 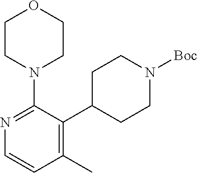 [SMILES: Cc1ccnc(N2CCOCC2)c1C1CCN(C(=O)OC(C)(C)C)CC1] | tert-butyl 4-(4-methyl-2- morpholinopyridin-3-yl)piperidine-1- carboxylate |
| B15-1 | 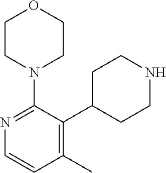 [SMILES: Cc1ccnc(N2CCOCC2)c1C1CCNCC1] | 4-(4-methyl-3-(piperidin-4-yl)pyridin-2- yl)morpholine |
| B16 | 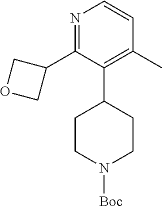 [SMILES: Cc1ccnc(C2COC2)c1C1CCN(C(=O)OC(C)(C)C)CC1] | tert-butyl 4-(4-methyl-2-(oxetan-3- yl)pyridin-3-yl)piperidine-1-carboxylate |
| B16-1 | 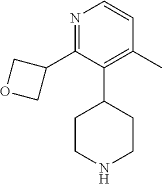 [SMILES: Cc1ccnc(C2COC2)c1C1CCNCC1] | 4-methyl-2-(oxetan-3-yl)-3-(piperidin-4- yl)pyridine |
| B17 | 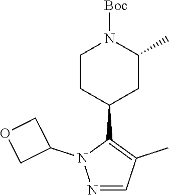 [SMILES: Cc1cnn(C2COC2)c1[C@H]1CCN(C(=O)OC(C)(C)C)[C@H](C)C1] | tert-butyl (2R,4S)-2-methyl-4-(4-methyl- 1-(oxetan-3-yl)-1H-pyrazol-5- yl)piperidine-1-carboxylate |
| B17′ | 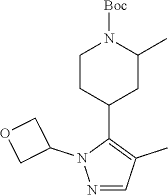 [SMILES: Cc1cnn(C2COC2)c1C1CCN(C(=O)OC(C)(C)C)C(C)C1] | tert-butyl 2-methyl-4-(4-methyl-1- (oxetan-3-yl)-1H-pyrazol-5-yl)piperidine- 1-carboxylate |
| B17-1 | 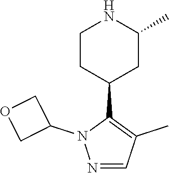 [SMILES: Cc1cnn(C2COC2)c1[C@H]1CCN[C@H](C)C1] | (2R,4S)-2-methyl-4-(4-methyl-1-(oxetan- 3-yl)-1H-pyrazol-5-yl)piperidine |
| B17-1′ | 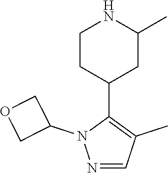 [SMILES: Cc1cnn(C2COC2)c1C1CCNC(C)C1] | 2-methyl-4-(4-methyl-1-(oxetan-3-yl)- 1H-pyrazol-5-yl)piperidine |
| B18 | 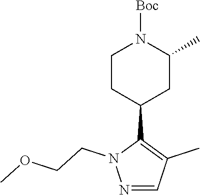 | tert-butyl (2R,4S)-4-(1-(2-methoxyethyl)- 4-methyl-1H-pyrazol-5-yl)-2- methylpiperidine-1-carboxylate |
| B18′ | 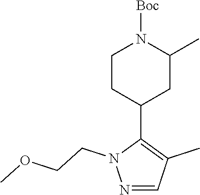 | tert-butyl 4-(1-(2-methoxyethyl)-4- methyl-1H-pyrazol-5-yl)-2- methylpiperidine-1-carboxylate |
| B18-1 | 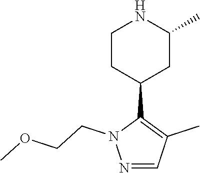 | (2R,4S)-4-(1-(2-methoxyethyl)-4-methyl- 1H-pyrazol-5-yl)-2-methylpiperidine |
| B18-1′ | 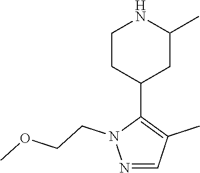 | 4-(1-(2-methoxyethyl)-4-methyl-1H- pyrazol-5-yl)-2-methylpiperidine |
| B19 | 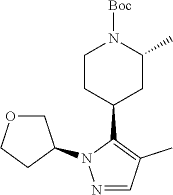 [SMILES: Cc1cnn([C@H]2CCOC2)c1[C@H]1CCN(C(=O)OC(C)(C)C)[C@H](C)C1] | tert-butyl (2R,4S)-2-methyl-4-(4-methyl- 1-((S)-tetrahydrofuran-3-yl)-1H-pyrazol- 5-yl)piperidine-1-carboxylate |
| B19′ | 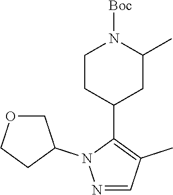 [SMILES: Cc1cnn(C2CCOC2)c1C1CCN(C(=O)OC(C)(C)C)C(C)C1] | tert-butyl 2-methyl-4-(4-methyl-1- (tetrahydrofuran-3-yl)-1H-pyrazol-5- yl)piperidine-1-carboxylate |
| B19-1 | 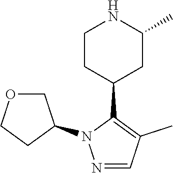 [SMILES: Cc1cnn([C@H]2CCOC2)c1[C@H]1CCN[C@H](C)C1] | (2R,4S)-2-methyl-4-(4-methyl-1-((S)- tetrahydrofuran-3-yl)-1H-pyrazol-5- yl)piperidine |
| B19-1′ | 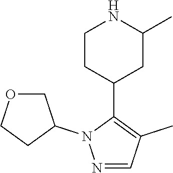 [SMILES: Cc1cnn(C2CCOC2)c1C1CCNC(C)C1] | 2-methyl-4-(4-methyl-1-(tetrahydrofuran- 3-yl)-1H-pyrazol-5-yl)piperidine |
| B20 | 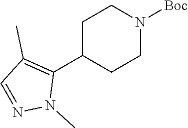 [SMILES: Cc1cnn(C)c1C1CCN(C(=O)OC(C)(C)C)CC1] | tert-butyl 4-(1,4-dimethyl-1H-pyrazol-5- yl)piperidine-1-carboxylate |
| B20-1 | 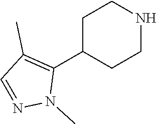 | 4-(1,4-dimethyl-1H-pyrazol-5- yl)piperidine |
| B21 | 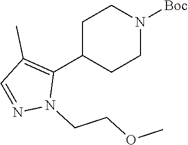 [SMILES: COCCn1ncc(C)c1C1CCN(C(=O)OC(C)(C)C)CC1] | tert-butyl 4-(1-(2-methoxyethyl)-4- methyl-1H-pyrazol-5-yl) piperidine-1-carboxylate |
| B21-1 | 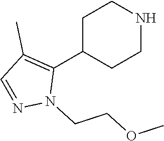 [SMILES: COCCn1ncc(C)c1C1CCNCC1] | 4-(1-(2-methoxyethyl)-4-methyl-1H- pyrazol-5-yl)piperidine |
| B22 | 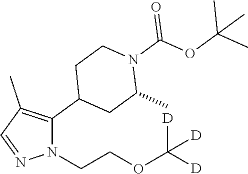 [SMILES: [2H]C([2H])([2H])OCCn1ncc(C)c1C1CCN(C(=O)OC(C)(C)C)[C@H](C)C1] | tert-butyl (2R,4S)-4-(1-(2-(methoxy- d3)ethyl)-4-methyl-1H-pyrazol-5-yl)-2- methylpiperidine-1-carboxylate |
| B22′ | 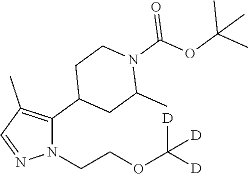 [SMILES: [2H]C([2H])([2H])OCCn1ncc(C)c1C1CCN(C(=O)OC(C)(C)C)C(C)C1] | tert-butyl 4-(1-(2-(methoxy-d3)ethyl)-4- methyl-1H-pyrazol-5-yl)-2- methylpiperidine-1-carboxylate |
| B22-1 | 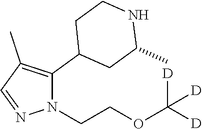 [SMILES: [2H]C([2H])([2H])OCCn1ncc(C)c1C1CCN[C@H](C)C1] | (2R,4S)-4-(1-(2-(methoxy-d3)ethyl)-4- methyl-1H-pyrazol-5-yl)-2- methylpiperidine |
| B22-1′ | 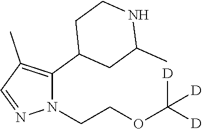 [SMILES: [2H]C([2H])([2H])OCCn1ncc(C)c1C1CCNC(C)C1] | 4-(1-(2-(methoxy-d3)ethyl)-4-methyl-1H- pyrazol-5-yl)-2-methylpiperidine |
| B23 | 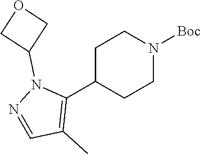 [SMILES: Cc1cnn(C2COC2)c1C1CCN(C(=O)OC(C)(C)C)CC1] | tert-butyl 4-(4-methyl-1-(oxetan-3-yl)- 1H-pyrazol-5-yl)piperidine-1-carboxylate |
| B23-1 | 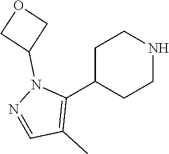 [SMILES: Cc1cnn(C2COC2)c1C1CCNCC1] | 4-(4-methyl-1-(oxetan-3-yl)-1H-pyrazol- 5-yl)piperidine |
| B24 | 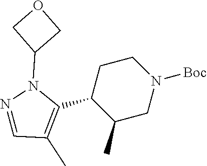 [SMILES: Cc1cnn(C2COC2)c1[C@@H]1CCN(C(=O)OC(C)(C)C)C[C@H]1C] | tert-butyl (3S,4R)-3-methyl-4-(4-methyl- 1-(oxetan-3-yl)-1H-pyrazol-5- yl)piperidine-1-carboxylate |
| B24′ | 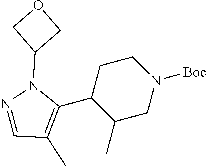 [SMILES: Cc1cnn(C2COC2)c1C1CCN(C(=O)OC(C)(C)C)CC1C] | tert-butyl 3-methyl-4-(4-methyl-1- (oxetan-3-yl)-1H-pyrazole-5-yl) piperidine-1-carboxylate |
| B24-1 | 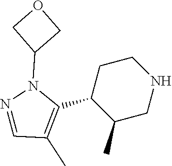 [SMILES: Cc1cnn(C2COC2)c1[C@@H]1CCNC[C@H]1C] | (3S,4R)-3-methyl-4-(4-methyl-1-(oxetan- 3-yl)-1H-pyrazol-5-yl)piperidine |
| B24-1′ | 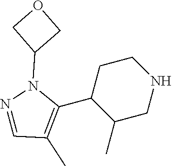 [SMILES: Cc1cnn(C2COC2)c1C1CCNCC1C] | 3-methyl-4-(4-methyl-1-(oxetan-3-yl)- 1H-pyrazol-5-yl)piperidine |
| B25 | 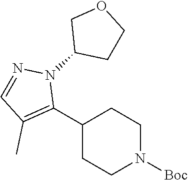 [SMILES: Cc1cnn([C@H]2CCOC2)c1C1CCN(C(=O)OC(C)(C)C)CC1] | tert-butyl (S)-4-(4-methyl-1- (tetrahydrofuran-3-yl)-1H-pyrazol-5- yl)piperidine-1-carboxylate |
| B25′ | 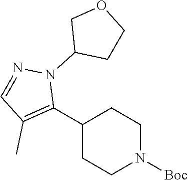 [SMILES: Cc1cnn(C2CCOC2)c1C1CCN(C(=O)OC(C)(C)C)CC1] | tert-butyl 4-(4-methyl-1-(tetrahydrofuran- 3-yl)-1H-pyrazol-5-yl) piperidine-1-carboxylate |
| B25-1 | 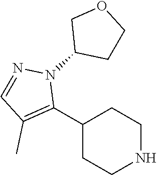 [SMILES: Cc1cnn([C@H]2CCOC2)c1C1CCNCC1] | (S)-4-(4-methyl-1-(tetrahydrofuran-3-yl)- 1H-pyrazol-5-yl)piperidine |
| B25-1′ | 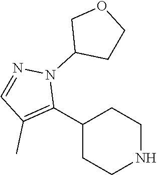 [SMILES: Cc1cnn(C2CCOC2)c1C1CCNCC1] | 4-(4-methyl-1-(tetrahydrofuran-3-yl)-1H- pyrazol-5-yl)piperidine |
| B26 | 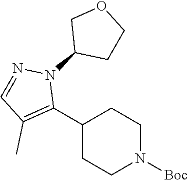 [SMILES: Cc1cnn([C@@H]2CCOC2)c1C1CCN(C(=O)OC(C)(C)C)CC1] | tert-butyl (R)-4-(4-methyl-1- (tetrahydrofuran-3-yl)-1H-pyrazol-5- yl)piperidine-1-carboxylate |
| B26-1 | 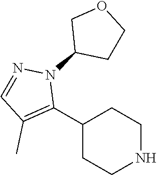 [SMILES: Cc1cnn([C@@H]2CCOC2)c1C1CCNCC1] | (R)-4-(4-methyl-1-(tetrahydrofuran-3-yl)- 1H-pyrazol-5-yl)piperidine |
| B27 | 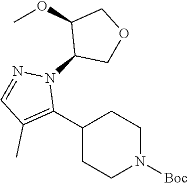 [SMILES: CO[C@H]1COC[C@H]1n1ncc(C)c1C1CCN(C(=O)OC(C)(C)C)CC1] | tert-butyl 4-(1-((3R,4R)-4- methoxytetrahydrofuran-3-yl)-4-methyl- 1H-pyrazol-5-yl)piperidine-1-carboxylate |
| B27′ | 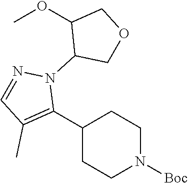 [SMILES: COC1COCC1n1ncc(C)c1C1CCN(C(=O)OC(C)(C)C)CC1] | tert-butyl 4-(1-(4- methoxytetrahydrofuran-3-yl)-4-methyl- 1H-pyrazol-5-yl)piperidine-1-carboxylate |
| B27-1 | 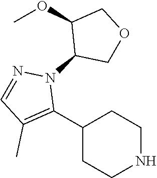 [SMILES: CO[C@H]1COC[C@H]1n1ncc(C)c1C1CCNCC1] | 4-(1-((3R,4R)-4-methoxytetrahydrofuran- 3-yl)-4-methyl-1H-pyrazol-5- yl)piperidine |
| B27-1′ | 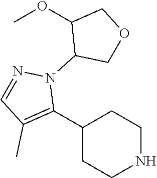 [SMILES: COC1COCC1n1ncc(C)c1C1CCNCC1] | 4-(1-(4-methoxytetrahydrofuran-3-yl)-4- methyl-1H-pyrazol-5-yl)piperidine |
| B28 | 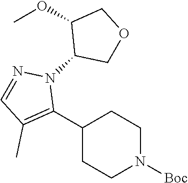 [SMILES: CO[C@@H]1COC[C@@H]1n1ncc(C)c1C1CCN(C(=O)OC(C)(C)C)CC1] | tert-butyl 4-(1-((3S,4S)-4- methoxytetrahydrofuran-3-yl)-4-methyl- 1H-pyrazol-5-yl)piperidine-1-carboxylate |
| B28-1 | 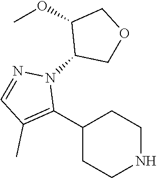 [SMILES: CO[C@@H]1COC[C@@H]1n1ncc(C)c1C1CCNCC1] | 4-(1-((3S,4S)-4-methoxytetrahydrofuran- 3-yl)-4-methyl-1H-pyrazol-5- yl)piperidine |
| B29 | 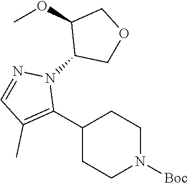 [SMILES: CO[C@H]1COC[C@@H]1n1ncc(C)c1C1CCN(C(=O)OC(C)(C)C)CC1] | tert-butyl 4-(1-((3S,4R)-4- methoxytetrahydrofuran-3-yl)-4-methyl- 1H-pyrazol-5-yl)piperidine-1-carboxylate |
| B29-1 | 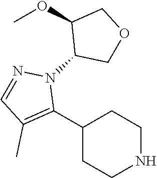 [SMILES: CO[C@H]1COC[C@@H]1n1ncc(C)c1C1CCNCC1] | 4-(1-((3S,4R)-4-methoxytetrahydrofuran- 3-yl)-4-methyl-1H-pyrazol-5- yl)piperidine |
| B30 | 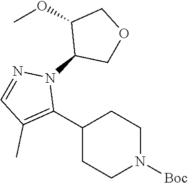 [SMILES: CO[C@@H]1COC[C@H]1n1ncc(C)c1C1CCN(C(=O)OC(C)(C)C)CC1] | tert-butyl 4-(1-((3R,4S)-4- methoxytetrahydrofuran-3-yl)-4-methyl- 1H-pyrazol-5-yl)piperidine-1-carboxylate |
| B30-1 | 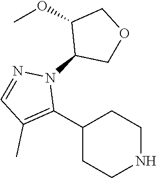 [SMILES: CO[C@@H]1COC[C@H]1n1ncc(C)c1C1CCNCC1] | 4-(1-((3R,4S)-4-methoxytetrahydrofuran- 3-yl)-4-methyl-1H-pyrazol-5- yl)piperidine |
| B31 | 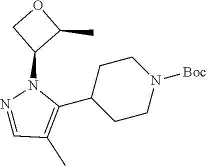 [SMILES: Cc1cnn([C@H]2CO[C@H]2C)c1C1CCN(C(=O)OC(C)(C)C)CC1] | tert-butyl 4-(4-methyl-1-((2S,3S)-2- methyloxetan-3-yl)-1H-pyrazol-5- yl)piperidine-1-carboxylate |
| B31′ | 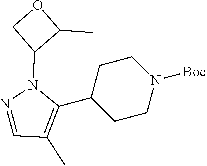 [SMILES: Cc1cnn(C2COC2C)c1C1CCN(C(=O)OC(C)(C)C)CC1] | tert-butyl 4-(4-methyl-1-(2-methyloxetan- 3-yl)-1H-pyrazol-5-yl)piperidine-1- carboxylate |
| B31-1 | 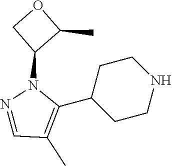 [SMILES: Cc1cnn([C@H]2CO[C@H]2C)c1C1CCNCC1] | 4-(4-methyl-1-((2R,3R)-2-methyloxetan- 3-yl)-1H-pyrazol-5-yl)piperidine |
| B31-1′ | 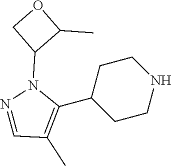 [SMILES: Cc1cnn(C2COC2C)c1C1CCNCC1] | 4-(4-methyl-1-(2-methyloxetan-3-yl)-1H- pyrazol-5-yl)piperidine |
| B32 | 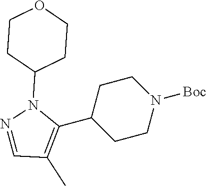 [SMILES: Cc1cnn(C2CCOCC2)c1C1CCN(C(=O)OC(C)(C)C)CC1] | tert-butyl 4-(4-methyl-1-(tetrahydro-2H- pyran-4-yl)-1H-pyrazol-5-yl)piperidine-1- carboxylate |
| B32-1 | 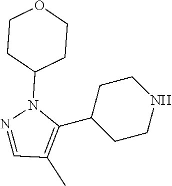 [SMILES: Cc1cnn(C2CCOCC2)c1C1CCNCC1] | 4-(4-methyl-1-(tetrahydro-2H-pyran-4- yl)-1H-pyrazol-5-yl)piperidine |
| B33 | 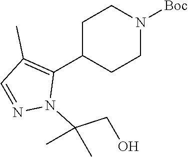 [SMILES: Cc1cnn(C(C)(C)CO)c1C1CCN(C(=O)OC(C)(C)C)CC1] | tert-butyl 4-(1-(1-hydroxy-2- methylpropan-2-yl)-4-methyl-1H-pyrazol- 5-yl)-3,6-dihydropyridine-1(2H)- carboxylate |
| B33-1 | 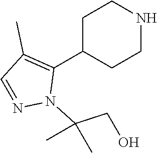 [SMILES: Cc1cnn(C(C)(C)CO)c1C1CCNCC1] | 2-methyl-2-(4-methyl-5-(piperidin-4-yl)- 1H-pyrazol-1-yl)propan-1-ol |
| B34 | 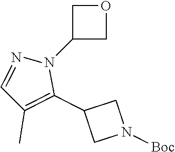 [SMILES: Cc1cnn(C2COC2)c1C1CN(C(=O)OC(C)(C)C)C1] | tert-butyl 3-(4-methyl-1-(oxetan-3-yl)- 1H-pyrazol-5-yl)azetidine-1-carboxylate |
| B34-1 | 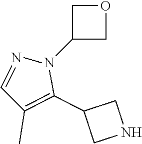 | 5-(azetidin-3-yl)-4-methyl-1-(oxetan-3- yl)-1H-pyrazole |
| B35 | 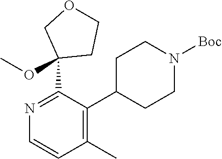 [SMILES: CO[C@]1(c2nccc(C)c2C2CCN(C(=O)OC(C)(C)C)CC2)CCOC1] | tert-butyl (S)-4-(2-(3- methoxytetrahydrofuran-3-yl)-4- methylpyridin-3-yl)piperidine-1- carboxylate |
| B35′ | 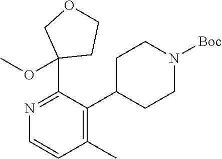 [SMILES: COC1(c2nccc(C)c2C2CCN(C(=O)OC(C)(C)C)CC2)CCOC1] | tert-butyl 4-(2-(3- methoxytetrahydrofuran-3-yl)-4- methylpyridin-3-yl)piperidine-1- carboxylate |
| B35-1 | 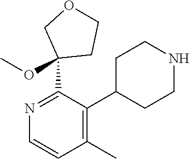 [SMILES: CO[C@]1(c2nccc(C)c2C2CCNCC2)CCOC1] | (S)-2-(3-methoxytetrahydrofuran-3-yl)-4- methyl-3-(piperidin-4-yl)pyridine |
| B35-1′ | 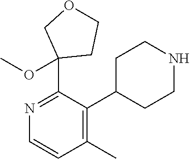 [SMILES: COC1(c2nccc(C)c2C2CCNCC2)CCOC1] | 2-(3-methoxytetrahydrofuran-3-yl)-4- methyl-3-(piperidin-4-yl)pyridine |
| B36 | 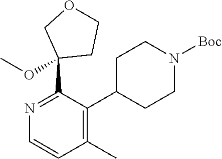 [SMILES: CO[C@@]1(c2nccc(C)c2C2CCN(C(=O)OC(C)(C)C)CC2)CCOC1] | tert-butyl (R)-4-(2-(3- methoxytetrahydrofuran-3-yl)-4- methylpyridin-3-yl)piperidine-1- carboxylate |
| B36-1 | 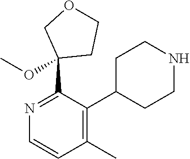 [SMILES: CO[C@@]1(c2nccc(C)c2C2CCNCC2)CCOC1] | (R)-2-(3-methoxytetrahydrofuran-3-yl)-4- methyl-3-(piperidin-4-yl)pyridine |
| B37 | 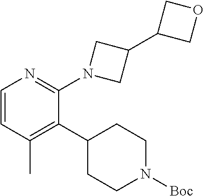 [SMILES: Cc1ccnc(N2CC(C3COC3)C2)c1C1CCN(C(=O)OC(C)(C)C)CC1] | tert-butyl 4-(4-methyl-2-(3-(oxetan-3- yl)azetidin-1-yl)pyridin-3-yl)piperidine-1- carboxylate |
| B37-1 | 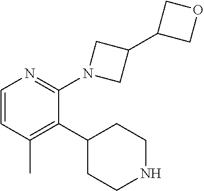 [SMILES: Cc1ccnc(N2CC(C3COC3)C2)c1C1CCNCC1] | 4-methyl-2-(3-(oxetan-3-yl)azetidin-1-yl)- 3-(piperidin-4-yl)pyridine |
| B38 | 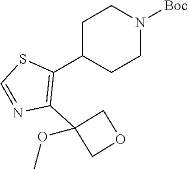 [SMILES: COC1(c2ncsc2C2CCN(C(=O)OC(C)(C)C)CC2)COC1] | tert-butyl 4-(4-(3-methoxyoxetan-3- yl)thiazol-5-yl)piperidine-1-carboxylate |
| B38-1 | 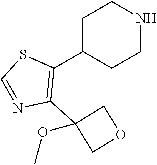 [SMILES: COC1(c2ncsc2C2CCNCC2)COC1] | 4-(3-methoxyoxetan-3-yl)-5-(piperidin-4- yl)thiazole |
| B39 | 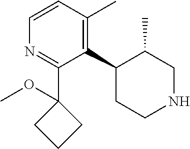 [SMILES: COC1(c2nccc(C)c2[C@@H]2CCNC[C@H]2C)CCC1] | 2-(3-methoxyoxetan-3-yl)-4-methyl-3- ((3S,4R)-3-methylpiperidin-4-yl)pyridine |
| B39′ | 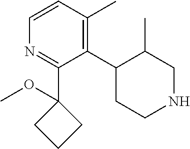 [SMILES: COC1(c2nccc(C)c2C2CCNCC2C)CCC1] | 2-(3-methoxyoxetan-3-yl)-4-methyl-3-(3- methylpiperidin-4-yl)pyridine |
| B40 | 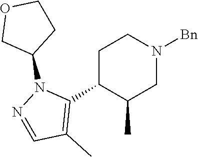 [SMILES: Cc1cnn([C@@H]2CCOC2)c1[C@@H]1CCN(Cc2ccccc2)C[C@H]1C] | (3S,4R)-1-benzyl-3-methyl-4-(4-methyl- 1-((R)-tetrahydrofuran-3-yl)-1H-pyrazol- 5-yl)piperidine |
| B40′ | 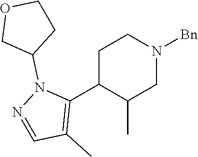 [SMILES: Cc1cnn(C2CCOC2)c1C1CCN(Cc2ccccc2)CC1C] | 1-benzyl-3-methyl-4-(4-methyl-1- (tetrahydrofuran-3-yl)-1H-pyrazol-5- yl)piperidine |
| B40-1 | 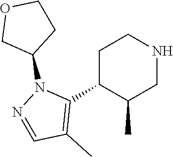 [SMILES: Cc1cnn([C@@H]2CCOC2)c1[C@@H]1CCNC[C@H]1C] | (3S,4R)-3-methyl-4-(4-methyl-1-((R)- tetrahydrofuran-3-yl)-1H-pyrazol-5- yl)piperidine |
| B40-1′ | 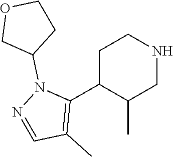 [SMILES: Cc1cnn(C2CCOC2)c1C1CCNCC1C] | 3-methyl-4-(4-methyl-1-(tetrahydrofuran- 3-yl)-1H-pyrazol-5-yl)piperidine |
| B41 | 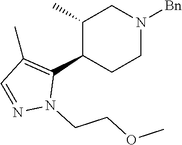 [SMILES: COCCn1ncc(C)c1[C@@H]1CCN(Cc2ccccc2)C[C@H]1C] | (3S,4R)-1-benzyl-4-(1-(2-methoxyethyl)- 4-methyl-1H-pyrazol-5-yl)-3- methylpiperidine |
| B41′ | 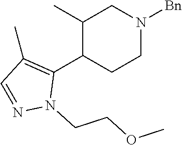 [SMILES: COCCn1ncc(C)c1C1CCN(Cc2ccccc2)CC1C] | 1-benzyl-4-(1-(2-methoxyethyl)-4- methyl-1H-pyrazol-5-yl)-3- methylpiperidine |
| B41-1 | 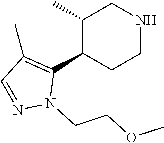 [SMILES: COCCn1ncc(C)c1[C@@H]1CCNC[C@H]1C] | (3S,4R)-4-(1-(2-methoxyethyl)-4-methyl- 1H-pyrazol-5-yl)-3-methylpiperidine |
| B41-1′ | 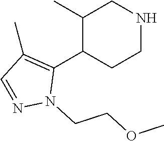 [SMILES: COCCn1ncc(C)c1C1CCNCC1C] | 4-(1-(2-methoxyethyl)-4-methyl-1H- pyrazol-5-yl)-3-methylpiperidine |
| B42 | 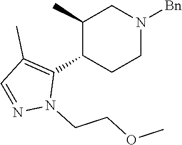 [SMILES: COCCn1ncc(C)c1[C@H]1CCN(Cc2ccccc2)C[C@@H]1C] | (3R,4S)-1-benzyl-4-(1-(2-methoxyethyl)- 4-methyl-1H-pyrazol-5-yl)-3- methylpiperidine |
| B42-1 | 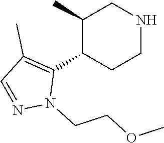 [SMILES: COCCn1ncc(C)c1[C@H]1CCNC[C@@H]1C] | (3R,4S)-4-(1-(2-methoxyethyl)-4-methyl- 1H-pyrazol-5-yl)-3-methylpiperidine |
| B43 | 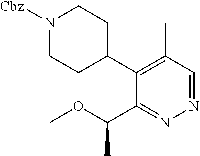 [SMILES: CO[C@H](C)c1nncc(C)c1C1CCN(C(=O)OCc2ccccc2)CC1] | tert-butyl (R)-4-(3-(1-methoxyethyl)-5- methylpyridazin-4-yl) piperidine-1-carboxylate |
| B43′ | 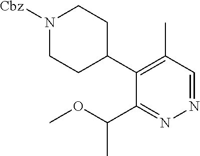 [SMILES: COC(C)c1nncc(C)c1C1CCN(C(=O)OCc2ccccc2)CC1] | benzyl 4-(3-(1-methoxyethyl)-5- methylpyridazin-4-yl) piperidine-1-carboxylate |
| B43-1 | 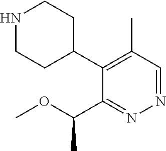 [SMILES: CO[C@H](C)c1nncc(C)c1C1CCNCC1] | (R)-3-(1-methoxyethyl)-5-methyl-4- (piperidin-4-yl)pyridazine |
| B43-1′ | 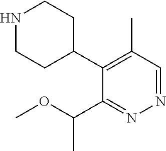 [SMILES: COC(C)c1nncc(C)c1C1CCNCC1] | 3-(1-methoxyethyl)-5-methyl-4- (piperidin-4-yl)pyridazine |
| B44 | 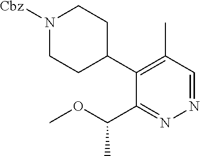 [SMILES: CO[C@@H](C)c1nncc(C)c1C1CCN(C(=O)OCc2ccccc2)CC1] | tert-butyl (S)-4-(3-(1-methoxyethyl)-5- methylpyridazin-4-yl)piperidine-1- carboxylate |
| B44′ | 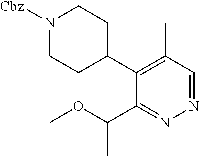 [SMILES: COC(C)c1nncc(C)c1C1CCN(C(=O)OCc2ccccc2)CC1] | tert-butyl 4-(3-(1-methoxyethyl)-5- methylpyridazin-4-yl) piperidine-1-carboxylate |
| B44-1 | 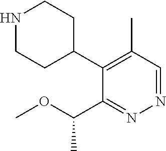 [SMILES: CO[C@@H](C)c1nncc(C)c1C1CCNCC1] | (S)-3-(1-methoxyethyl)-5-methyl-4- (piperidin-4-yl)pyridazine |
| B44-1′ | 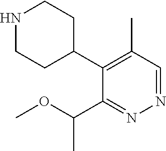 [SMILES: COC(C)c1nncc(C)c1C1CCNCC1] | 3-(1-methoxyethyl)-5-methyl-4- (piperidin-4-yl)pyridazine |
| B45 | 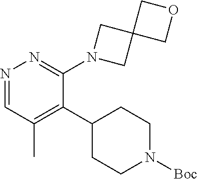 [SMILES: Cc1cnnc(N2CC3(COC3)C2)c1C1CCN(C(=O)OC(C)(C)C)CC1] | benzyl 4-(5-methyl-3-(2-oxa-6- azaspiro[3.3]heptan-6-yl)pyridazin-4- yl)piperidine-1-carboxylate |
| B45-1 | 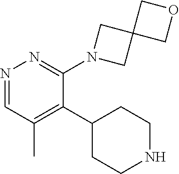 [SMILES: Cc1cnnc(N2CC3(COC3)C2)c1C1CCNCC1] | 6-(5-methyl-4-(piperidin-4-yl)pyridazin- 3-yl)-2-oxa-6-azaspiro[3.3]heptane |
| B46 | 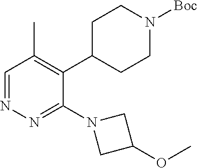 [SMILES: COC1CN(c2nncc(C)c2C2CCN(C(=O)OC(C)(C)C)CC2)C1] | tert-butyl 4-(3-(3-methoxyazetidin-1-yl)- 5-methylpyridazin-4-yl) piperidine-1-carboxylate |
| B46-1 | 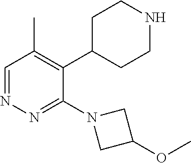 [SMILES: COC1CN(c2nncc(C)c2C2CCNCC2)C1] | 3-(3-methoxyazetidin-1-yl)-5-methyl-4- (piperidin-4-yl)pyridazine |

Also provided herein are intermediates of Formula (Int-C):

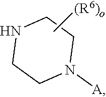

nitrogen-protected analogs thereof (e.g., a BOC-protected analog, such as

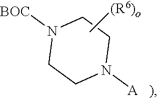

and pharmaceutically acceptable salts of any of the foregoing, wherein each of A, o, R⁶, R^{ZA}, and R^{ZB} is as previously defined herein for Int-B. For example, Formula (Int-C) includes intermediates of Formula (Int-CA):

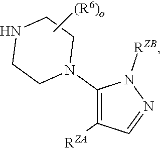

### Formula (Int-CB):

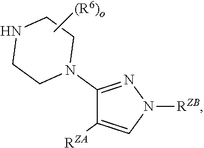

### Formula (Int-CC):

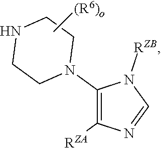

### Formula (Int-CD):

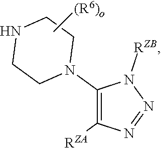

### Formula (Int-CE):

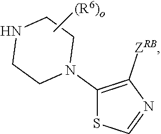

### Formula (Int-CF):

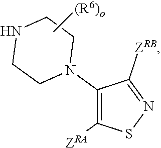

### Formula (Int-CG):

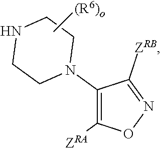

### Formula (Int-CH):

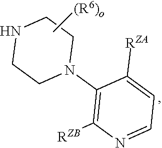

### Formula (Int-CI):

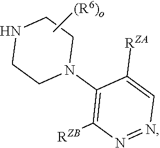

nitrogen-protected analogs of any of the foregoing (e.g., N-BOC protected analogs, such as

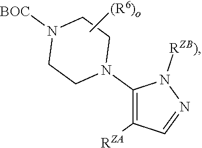

and pharmaceutically acceptable salts of any of the foregoing; wherein each of the substituents are as previously defined for Int-B. In some cases, the disclosure provides an intermediate of Formula (Int-CA):

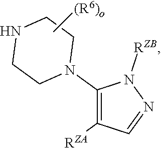

a nitrogen-protected analog thereof, or a pharmaceutically acceptable salt thereof. Contemplated examples intermediates of Formula (Int-C) are listed in Table INT-C, and include pharmaceutically acceptable salts thereof.

TABLE INT-C

| Intermediate Ref. | Structure | Name |
|---|---|---|
| C1 | 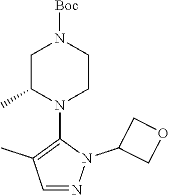 [SMILES: Cc1cnn(C2COC2)c1N1CCN(C(=O)OC(C)(C)C)C[C@H]1C] | tert-butyl (R)-3-methyl-4-(4-methyl-1- (oxetan-3-yl)-1H-pyrazol-5-yl)piperazine- 1-carboxylate |
| C1′ | 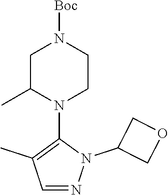 [SMILES: Cc1cnn(C2COC2)c1N1CCN(C(=O)OC(C)(C)C)CC1C] | tert-butyl 3-methyl-4-(4-methyl-1- (oxetan-3-yl)-1H-pyrazol-5-yl)piperazine- 1-carboxylate |
| C1-1 | 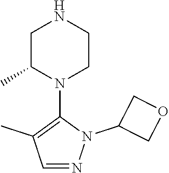 [SMILES: Cc1cnn(C2COC2)c1N1CCNC[C@H]1C] | (R)-2-methyl-1-(4-methyl-1-(oxetan-3- yl)-1H-pyrazol-5-yl)piperazine |
| C1-1′ | 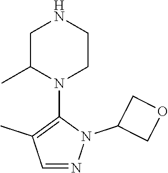 [SMILES: Cc1cnn(C2COC2)c1N1CCNCC1C] | 2-methyl-1-(4-methyl-1-(oxetan-3-yl)- 1H-pyrazol-5-yl)piperazine |

Further provided herein are intermediates of Formula (Int-D):

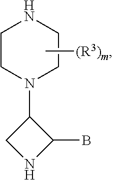

nitrogen-protected analogs thereof (e.g., a BOC-protected analog, such as

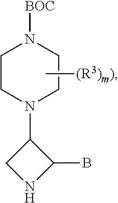

and pharmaceutically acceptable salts of any of the foregoing (e.g., TFA salt), wherein m and R³ are each as defined herein in the COMPOUNDS OF FORMULA (II) section, and B is C₁₋₃alkylene-CH═CH₂ or C₁₋₃alkyleneOH. In some cases, m is 0 or 1; R³ is CH₃. In some cases, B is CH₂CH═CH₂ or CH₂CH₂OH. Contemplated examples of intermediates of Formula (Int-D) are listed in Table INT-D and include nitrogen analogs of the compounds listed in Table INT-D, and pharmaceutically acceptable salts of compounds listed in Table INT-D.

TABLE INT-D

| Intermediate | | |
|---|---|---|
| Ref. | Structure | Name |
| D1 | 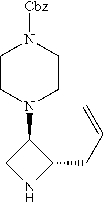 [SMILES: C=CC[C@@H]1NC[C@H]1N1CCN(C(=O)OCc2ccccc2)CC1] | benzyl 4-((2S,3R)-2-allylazetidin-3- yl)piperazine-1-carboxylate |
| D1′ | 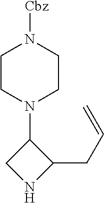 [SMILES: C=CCC1NCC1N1CCN(C(=O)OCc2ccccc2)CC1] | benzyl 4-(2-allylazetidin-3-yl)piperazine- 1-carboxylate |
| D1-1 | 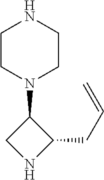 | 1-((2S,3R)-2-allylazetidin-3-yl)piperazine |
| D1-1′ | 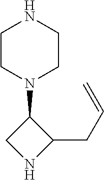 | 1-((3R)-2-allylazetidin-3-yl)piperazine |
| D2 | 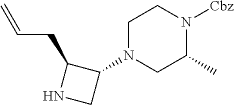 [SMILES: C=CC[C@@H]1NC[C@H]1N1CCN(C(=O)OCc2ccccc2)[C@H](C)C1] | benzyl (R)-4-((2S,3R)-2-allylazetidin-3- yl)-2-methylpiperazine-1-carboxylate |
| D2′ | 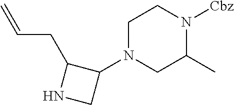 [SMILES: C=CCC1NCC1N1CCN(C(=O)OCc2ccccc2)C(C)C1] | benzyl 4-(2-allylazetidin-3-yl)-2- methylpiperazine-1-carboxylate |
| D2″ | 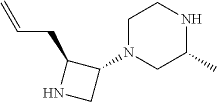 | (R)-1-((2S,3R)-2-allylazetidin-3-yl)-3- methylpiperazine |
| D2′′′ | 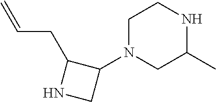 | 1-(2-allylazetidin-3-yl)-3- methylpiperazine |
| D3 | 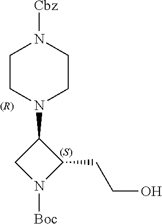 | benzyl 4-((2S,3R)-2-(3- hydroxypropyl)azetidin-3-yl)piperazine- 1-carboxylate |
| D3′ | 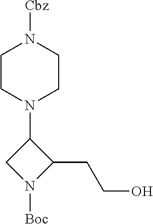 [SMILES: CC(C)(C)OC(=O)N1CC(N2CCN(C(=O)OCc3ccccc3)CC2)C1CCO] | benzyl 4-(1-(tert-butoxycarbonyl)-2-(2- hydroxyethyl)azetidin-3-yl)piperazine-1- carboxylate |
| D3-1 | 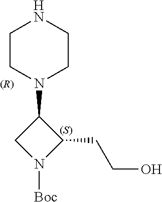 | tert-butyl (2S,3R)-2-(2-hydroxyethyl)-3- (piperazin-1-yl)azetidine-1-carboxylate |
| D3-1′ | 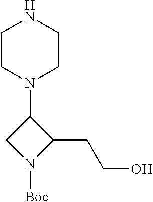 [SMILES: CC(C)(C)OC(=O)N1CC(N2CCNCC2)C1CCO] | tert-butyl 2-(2-hydroxyethyl)-3- (piperazin-1-yl)azetidine-1-carboxylate |

Further provided herein are intermediates of Formula (Int-E):

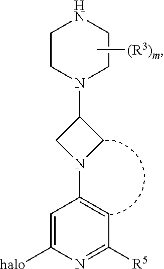

nitrogen-protected analogs thereof, or pharmaceutically acceptable salts of any of the foregoing, wherein halo is F, Cl, Br, or I, and m, [symbol] R³, and R⁵ are as defined herein in the COMPOUNDS OF FORMULA (II) section. In some cases:
m is 0, 1, 2, 3, or 4;
[symbol] is C₂₋₆alkylene, C₃₋₆alkenylene, heteroalkylene having 2-6 total atoms and 1-3 heteroatoms selected from N, O, and S, or heteroalkenylene having 3-6 total atoms and 1 or 2 heteroatoms selected from N, O, and S, wherein [symbol] is unsubstituted or substituted with 1-4 substituents, and each substituent independently is C₁₋₃alkyl, C₁₋₃haloalkyl, C₂₋₃alkenyl, halo, CN, C₀₋₃alkyleneOH, C₀₋₆alkylene-C₁₋₃alkoxy, C₃₋₃cycloalkyl, C₄₋₅cycloalkenyl, heterocycloalkyl having 4 or 5 total ring atoms and 1-3 heteroatoms selected from N, O, and S, heterocycloalkenyl having 4 or 5 total ring atoms and 1-3 heteroatoms selected from N, O, and S, or phenyl; or two geminal substituents, together with the atom to which they are attached, form oxo, =CH₂, spiro-C₃₋₅cycloalkyl, spiro-C₄₋₅cycloalkenyl, spiro-heterocycloalkyl having 3-5 total ring atoms and 1 or 2 heteroatoms selected from N, O and S, or spiro-heterocycloalkenyl having 4 or 5 total ring atoms and 1 or 2 heteroatoms selected from N, O and S; or two vicinal substituents, together with the atoms to which they are attached, form fused-C₃₋₅cycloalkyl, fused-C₄₋₅cycloalkenyl, fused-heterocycloalkyl having 3-5 total ring atoms and 1 or 2 heteroatoms selected from N. O and S or fused-heterocycloalkenyl having 4 or 5 total ring atoms and 1 or 2 heteroatoms selected from N, O and S;
each R³ independently is C₁₋₃alkyl, C₁₋₄haloalkyl,

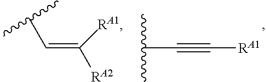

C₀₋₃alkyleneCN, C₀₋₃alkyleneOH, or C₀₋₃alkylene-C₁₋₃alkoxy; or two geminal R³, together with the atom to which they are attached, form oxo, spiro-C₁₋₃-cycloalkyl, spiro-C₄₋₇cycloalkenyl, spiro-heterocycloalkyl having 3-7 total ring atoms and 1 or 2 heteroatoms selected from N, O, and S, or spiro-heterocycloalkenyl having 4-7 total ring atoms and 1 or 2 heteroatoms selected from N, O, and S; or two vicinal R³, together with the atoms to which they are attached, form fused-C₃₋₇cycloalkyl, fused-C₄₋₇cycloalkenyl, fused-heterocycloalkyl having 3-7 total ring atoms and 1 or 2 heteroatoms selected from N, O, and S, or fused-heterocycloalkenyl having 4-7 total ring atoms and 1 or 2 heteroatoms selected from N, O, and S; and
R⁵ is halo, C₁₋₄haloalkyl, C₁₋₆alkyl, C₂₋₄alkenyl, C₂₋₄alkynyl, C₁₋₃alkoxy, C₁₋₃thioalkyl, C₃₋₇cycloalkyl, C₅₋₇cycloalkenyl, heterocycloalkyl having 3-7 total ring atoms and 1-3 heteroatoms selected from N, O, and S, or heterocycloalkenyl having 5-7 total ring atoms and 1-3 heteroatoms selected from N, O, and S, wherein each of the foregoing independently is unsubstituted or substituted with 1-3 substituents, and each substituent independently is C₁₋₃haloalkyl, C₀₋₆alkylene-OH, C₀₋₆alkylene-C₁₋₃alkoxy, C₃₋₇cycloalkyl, C₅₋₇cycloalkenyl, heterocycloalkyl having 3-7 total ring atoms and 1-3 heteroatoms selected from N, O, and S, heterocycloalkenyl having 4-7 total ring atoms and 1-3 heteroatoms selected from N, O, and S, or phenyl.
   In some cases, halo is Cl, m is 0 or 1; R³ is CH₃; and R⁵ is CHF₂ or CF₃. In some cases, n is 0 and R⁵ is CHF₂. In some cases, [symbol] is

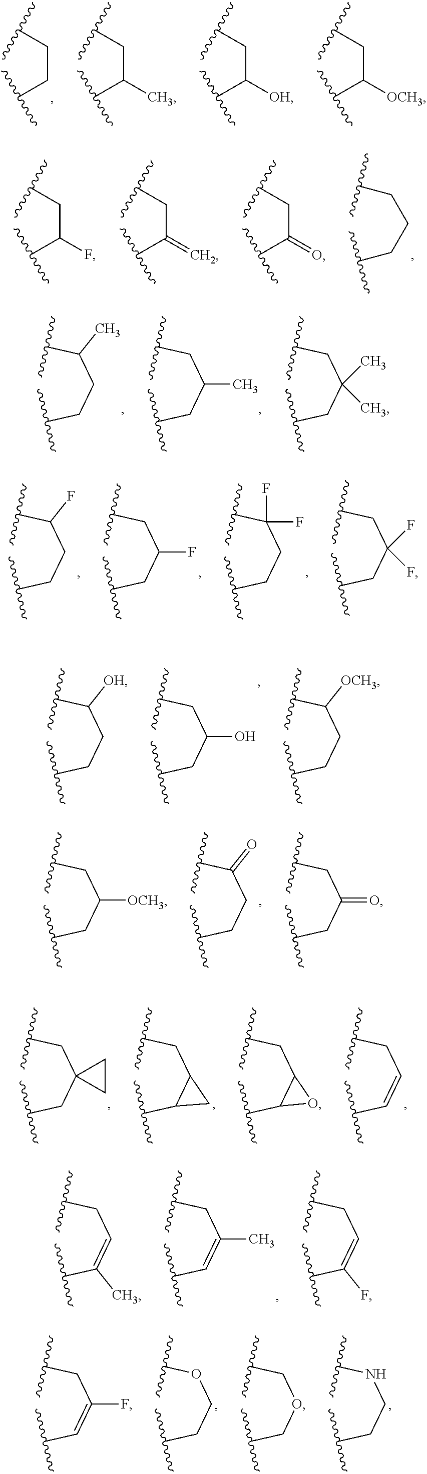

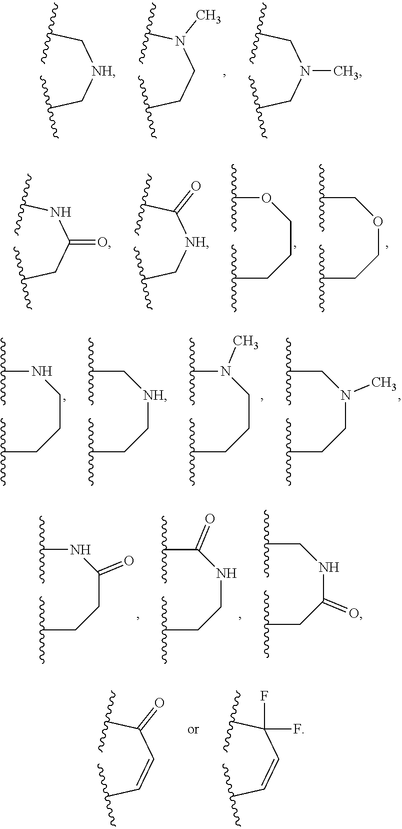

In some cases, [symbol] is

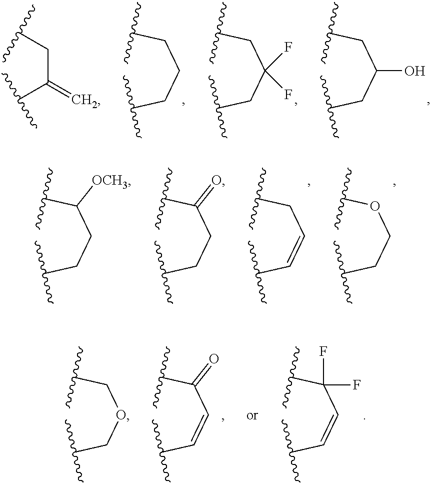

Contemplated examples of intermediate of Formula (Int-E) are listed in Table INT-E, below, and include pharmaceutically acceptable salts thereof.

TABLE INT-3

| Intermediate | | |
|---|---|---|
| Ref. | Structure | Name |
| E1 | 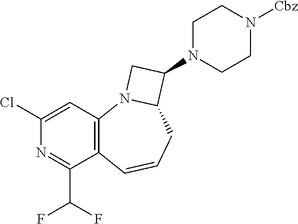 [SMILES: O=C(OCc1ccccc1)N1CCN([C@@H]2CN3c4cc(Cl)nc(C(F)F)c4C=CC[C@@H]23)CC1] | benzyl 4-((7aS,8R)-2-chloro-4- (difluoromethyl)-7,7a,8,9- tetrahydroazeto[1,2-a]pyrido[3,4- f]azepin-8-yl)piperazine-1-carboxylate |
| E1′ | 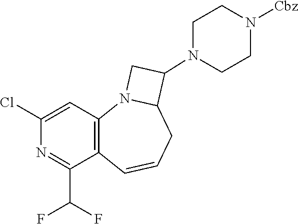 [SMILES: O=C(OCc1ccccc1)N1CCN(C2CN3c4cc(Cl)nc(C(F)F)c4C=CCC23)CC1] | benzyl 4-(2-chloro-4-(difluoromethyl)- 7,7a,8,9-tetrahydroazeto[1,2-a]pyrido[3,4- f]azepin-8-yl)piperazine-1-carboxylate |
| E1-1 | 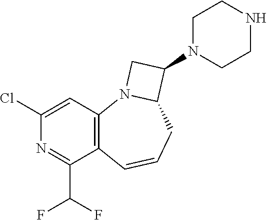 [SMILES: FC(F)c1nc(Cl)cc2c1C=CC[C@H]1[C@H](N3CCNCC3)CN21] | (7aS,8R)-2-chloro-4-(difluoromethyl)-8- (piperazin-1-yl)-7,7a,8,9- tetrahydroazeto[1,2-a]pyrido[3,4- f]azepine |
| E1-1′ | 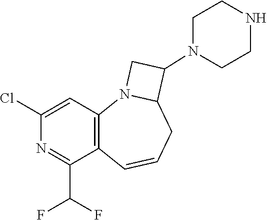 [SMILES: FC(F)c1nc(Cl)cc2c1C=CCC1C(N3CCNCC3)CN21] | 2-chloro-4-(difluoromethyl)-8-(piperazin- 1-yl)-7,7a,8,9-tetrahydroazeto[1,2- a]pyrido[3,4-f]azepine |
| E2 | 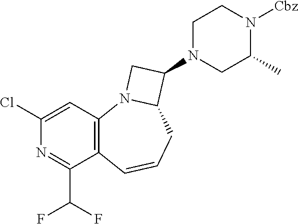 [SMILES: C[C@@H]1CN([C@@H]2CN3c4cc(Cl)nc(C(F)F)c4C=CC[C@@H]23)CCN1C(=O)OCc1ccccc1] | benzyl (R)-4-((7aS,8R)-2-chloro-4- (difluoromethyl)-7,7a,8,9- tetrahydroazeto[1,2-a]pyrido[3,4- f]azepin-8-yl)-2-methylpiperazine-1- carboxylate |
| E2′ | 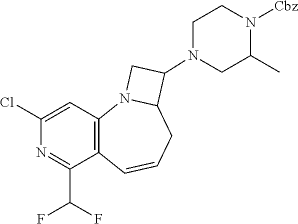 [SMILES: CC1CN(C2CN3c4cc(Cl)nc(C(F)F)c4C=CCC23)CCN1C(=O)OCc1ccccc1] | benzyl 4-(2-chloro-4-(difluoromethyl)- 7,7a,8,9-tetrahydroazeto[1,2-a]pyrido[3,4- f]azepin-8-yl)-2-methylpiperazine-1- carboxylate |
| E2-1 | 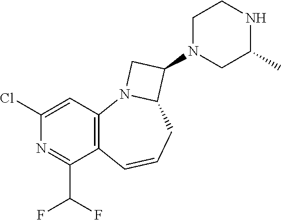 [SMILES: C[C@@H]1CN([C@@H]2CN3c4cc(Cl)nc(C(F)F)c4C=CC[C@@H]23)CCN1] | (7aS,8R)-2-chloro-4-(difluoromethyl)-8- ((R)-3-methylpiperazin-1-yl)-7,7a,8,9- tetrahydroazeto[1,2-a]pyrido[3,4- f]azepine |
| E2-1′ | 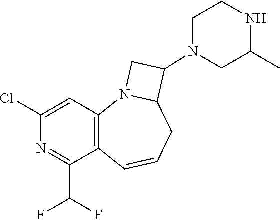 [SMILES: CC1CN(C2CN3c4cc(Cl)nc(C(F)F)c4C=CCC23)CCN1] | 2-chloro-4-(difluoromethyl)-8-(3- methylpiperazin-1-yl)-7,7a,8,9- tetrahydroazeto[1,2-a]pyrido[3,4- f]azepine |
| E3 | 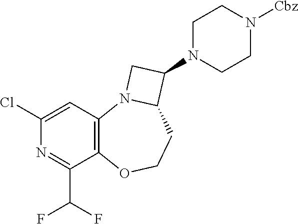 [SMILES: O=C(OCc1ccccc1)N1CCN([C@@H]2CN3c4cc(Cl)nc(C(F)F)c4OCC[C@@H]23)CC1] | benzyl 4-((7aS,8R)-2-chloro-4- (difluoromethyl)-7,7a,8,9-tetrahydro-6H- azeto[1,2-d]pyrido[3,4-b][1,4]oxazepin-8- yl)piperazine-1-carboxylate |
| E3′ | 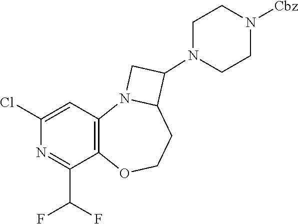 [SMILES: O=C(OCc1ccccc1)N1CCN(C2CN3c4cc(Cl)nc(C(F)F)c4OCCC23)CC1] | benzyl 4-(2-chloro-4-(difluoromethyl)- 7,7a,8,9-tetrahydro-6H-azeto[1,2- d]pyrido[3,4-b][1,4]oxazepin-8- yl)piperazine-1-carboxylate |
| E3-1 | 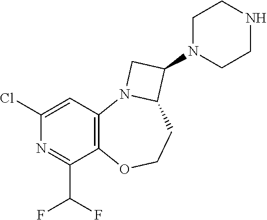 [SMILES: FC(F)c1nc(Cl)cc2c1OCC[C@H]1[C@H](N3CCNCC3)CN21] | (7aS,8R)-2-chloro-4-(difluoromethyl)-8- (piperazin-1-yl)-7,7a,8,9-tetrahydro-6H- azeto[1,2-d]pyrido[3,4-b][1,4]oxazepine |
| E3-1′ | 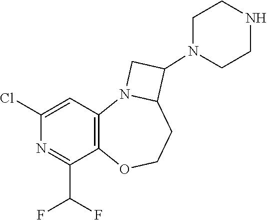 [SMILES: FC(F)c1nc(Cl)cc2c1OCCC1C(N3CCNCC3)CN21] | 2-chloro-4-(difluoromethyl)-8-(piperazin- 1-yl)-7,7a,8,9-tetrahydro-6H-azeto[1,2- d]pyrido[3,4-b][1,4]oxazepine |
| E4 | 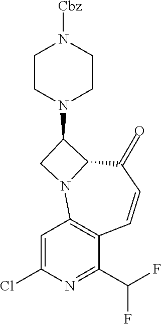 | Benzyl 4-((7aR,8R)-2-chloro-4- (difluoromethyl)-7-oxo-7,7a,8,9- tetrahydroazeto[1,2-a]pyrido[3,4- f]azepin-8-yl)piperazine-1-carboxylate |
| E4′ | 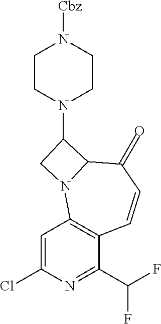 | benzyl 4-(2-chloro-4-(difluoromethyl)-7- oxo-7,7a,8,9- tetrahydroazeto[1,2-a]pyrido[3,4- f]azepin-8-yl)piperazine-1-carboxylate |
| E4-1 | 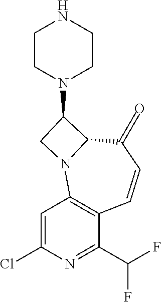 | (7aR,8R)-2-chloro-4-(difluoromethyl)-8- (piperazin-1-yl)-8,9-dihydroazeto[1,2- a]pyrido[3,4-f]azepin-7(7aH)-one |
| E4-1′ | 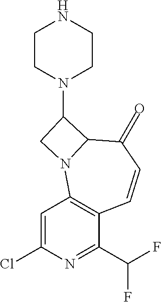 | 2-chloro-4-(difluoromethyl)-8-(piperazin- 1-yl)-8,9-dihydroazeto[1,2-a]pyrido[3,4- f]azepin-7(7aH)-one |
| E5 | 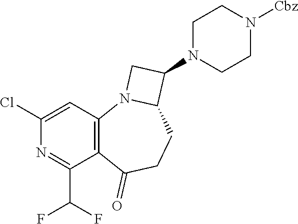 [SMILES: O=C1CC[C@H]2[C@H](N3CCN(C(=O)OCc4ccccc4)CC3)CN2c2cc(Cl)nc(C(F)F)c21] | benzyl 4-((7aS,8R)-2-chloro-4- (difluoromethyl)-5-oxo-5,6,7,7a,8,9- hexahydroazeto[1,2-a]pyrido[3,4- f]azepin-8-yl)piperazine-1-carboxylate |
| E5′ | 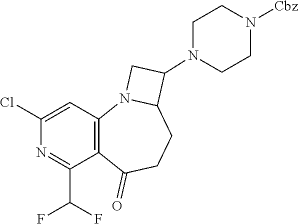 [SMILES: O=C1CCC2C(N3CCN(C(=O)OCc4ccccc4)CC3)CN2c2cc(Cl)nc(C(F)F)c21] | benzyl 4-(2-chloro-4-(difluoromethyl)-5- oxo-5,6,7,7a,8,9-hexahydroazeto[1,2- a]pyrido[3,4-f]azepin-8-yl)piperazine-1- carboxylate |
| E5-1 | 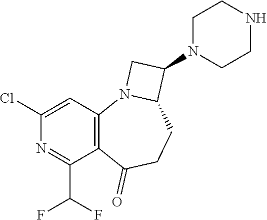 [SMILES: O=C1CC[C@H]2[C@H](N3CCNCC3)CN2c2cc(Cl)nc(C(F)F)c21] | (7aS,8R)-2-chloro-4-(difluoromethyl)-8- (piperazin-1-yl)-7,7a,8,9- tetrahydroazeto[1,2-a]pyrido[3,4- f]azepin-5(6H)-one |
| E5-1′ | 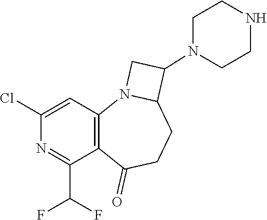 [SMILES: O=C1CCC2C(N3CCNCC3)CN2c2cc(Cl)nc(C(F)F)c21] | 2-chloro-4-(difluoromethy)-8-(piperazin- 1-yl)-7,7a,8,9-tetrahydroazeto[1,2- a]pyrido[3,4-f]azepin-5(6H)-one |
| E6 | 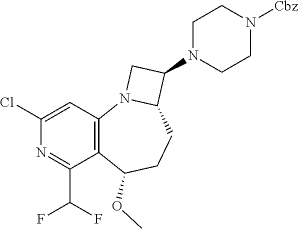 [SMILES: CO[C@H]1CC[C@H]2[C@H](N3CCN(C(=O)OCc4ccccc4)CC3)CN2c2cc(Cl)nc(C(F)F)c21] | benzyl 4-((5S,7aS,8R)-2-chloro-4- (difluoromethyl)-5-methoxy-5,6,7,7a,8,9- hexahydroazeto[1,2-a]pyrido[3,4- f]azepin-8-yl)piperazine-1-carboxylate |
| E6′ | 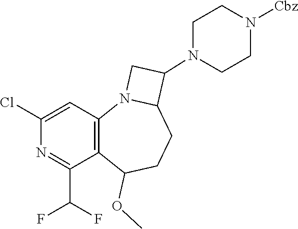 [SMILES: COC1CCC2C(N3CCN(C(=O)OCc4ccccc4)CC3)CN2c2cc(Cl)nc(C(F)F)c21] | benzyl 4-(2-chloro-4-(difluoromethyl)-5- methoxy-5,6,7,7a,8,9- hexahydroazeto[1,2-a]pyrido[3,4- f]azepin-8-yl)piperazine-1-carboxylate |
| E6-1 | 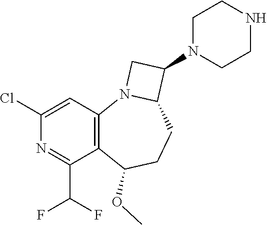 [SMILES: CO[C@H]1CC[C@H]2[C@H](N3CCNCC3)CN2c2cc(Cl)nc(C(F)F)c21] | (5S,7aS,8R)-2-chloro-4-(difluoromethyl)- 5-methoxy-8-(piperazin-1-yl)- 5,6,7,7a,8,9-hexahydroazeto[1,2- a]-pyrido[3,4-f]azepine |
| E6-1′ | 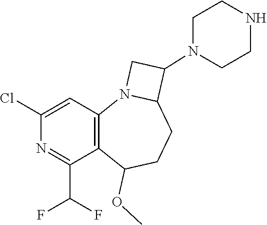 [SMILES: COC1CCC2C(N3CCNCC3)CN2c2cc(Cl)nc(C(F)F)c21] | 2-chloro-4-(difluoromethyl)-5-methoxy- 8-(piperazin-1-yl)-5,6,7,7a,8,9- hexahydroazeto[1,2-a]pyrido[3,4- f]azepine |
| E7 | 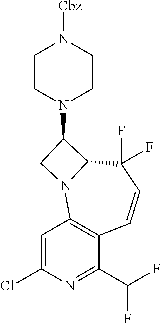 [SMILES: O=C(OCc1ccccc1)N1CCN([C@@H]2CN3c4cc(Cl)nc(C(F)F)c4C=CC(F)(F)[C@@H]23)CC1] | Benzyl 4-((7aR,8R)-2-chloro-4- (difluoromethyl)-7,7-difluoro-7,7a,8,9- tetrahydroazeto[1,2-a]pyrido[3,4- f]azepin-8-yl)piperazine-1-carboxylate |
| E7′ | 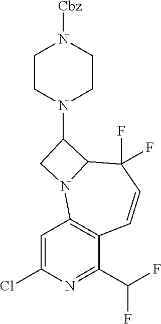 [SMILES: O=C(OCc1ccccc1)N1CCN(C2CN3c4cc(Cl)nc(C(F)F)c4C=CC(F)(F)C23)CC1] | benzyl 4-(2-chloro-4-(difluoromethyl)- 7,7-difluoro-7,7a,8,9-tetrahydroazeto[1,2- a]pyrido[3,4-f]azepin-8-yl)piperazine-1- carboxylate |
| E7-1 | 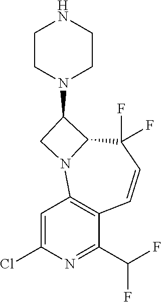 [SMILES: FC(F)c1nc(Cl)cc2c1C=CC(F)(F)[C@H]1[C@H](N3CCNCC3)CN21] | (7aR,8R)-2-chloro-4-(difluoromethyl)- 7,7-difluoro-8-(piperazin-1-yl)-7,7a,8,9- tetrahydroazeto[1,2-a]pyrido[3,4- f]azepine |
| E7-1′ | 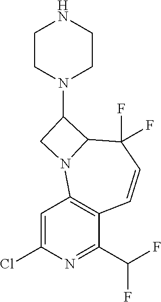 [SMILES: FC(F)c1nc(Cl)cc2c1C=CC(F)(F)C1C(N3CCNCC3)CN21] | 2-chloro-4-(difluoromethyl)-7,7-difluoro- 8-(piperazin-1-yl)-7,7a,8,9- tetrahydroazeto[1,2-a]pyrido[3,4- f]azepine |
| E8 | 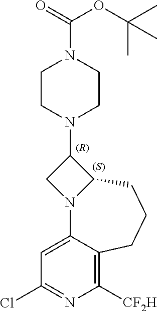 | tert-Butyl 4-((7aS,8R)-2-chloro-4- (difluoromethyl)-5,6,7,7a,8,9- hexahydroazeto[1,2-a]pyrido[3,4- f]azepin-8-yl)piperazine-1-carboxylate |
| E8′ | 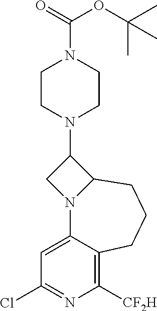 [SMILES: CC(C)(C)OC(=O)N1CCN(C2CN3c4cc(Cl)nc(C(F)F)c4CCCC23)CC1] | tert-butyl 4-(2-chloro-4-(difluoromethyl)- 5,6,7,7a,8,9-hexahydroazeto[1,2- a]pyrido[3,4-f]azepin-8-yl)piperazine-1- carboxylate |
| E8-1 | 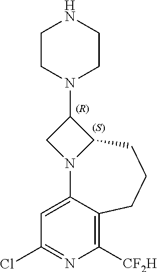 | (7aS,8R)-2-chloro-4-(difluoromethyl)-8- (piperazin-1-yl)-5,6,7,7a,8,9- hexahydroazeto[1,2-a]pyrido[3,4- f]azepine |
| E8-1′ | 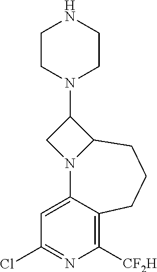 [SMILES: FC(F)c1nc(Cl)cc2c1CCCC1C(N3CCNCC3)CN21] | 2-chloro-4-(difluoromethyl)-8-(piperazin- 1-yl)-5,6,7,7a,8,9-hexahydroazeto[1,2- a]pyrido[3,4-f]azepine |
| E9 | 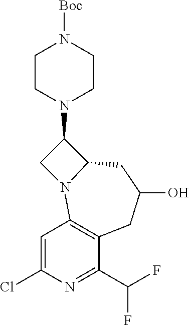 | tert-Butyl 4-((7aS,8R)-2-chloro-4- (difluoromethyl)-6-hydroxy-5,6,7,7a,8,9- hexahydroazeto[1,2-a]pyrido[3,4- f]azepin-8-yl)piperazine-1-carboxylate |
| E9′ | 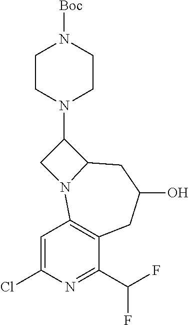 [SMILES: CC(C)(C)OC(=O)N1CCN(C2CN3c4cc(Cl)nc(C(F)F)c4CC(O)CC23)CC1] | tert-butyl 4-(2-chloro-4-(difluoromethyl)- 6-hydroxy-5,6,7,7a,8,9- hexahydroazeto[1,2-a]pyrido[3,4- f]azepin-8-yl)piperazine-1-carboxylate |
| E9-1 | 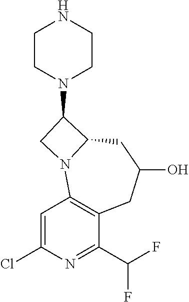 | (7aS,8R)-2-chloro-4-(difluoromethyl)-8- (piperazin-1-yl)-5,6,7,7a,8,9- hexahydroazeto[1,2-a]pyrido[3,4- f]azepin-6-ol |
| E9-1′ | 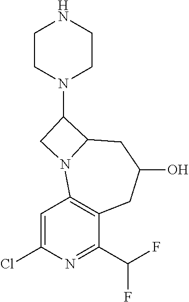 [SMILES: OC1Cc2c(cc(Cl)nc2C(F)F)N2CC(N3CCNCC3)C2C1] | 2-chloro-4-(difluoromethyl)-8-(piperazin- 1-yl)-5,6,7,7a,8,9-hexahydroazeto[1,2- a]pyrido[3,4-f]azepin-6-ol |
| E10 | 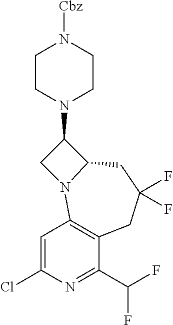 [SMILES: O=C(OCc1ccccc1)N1CCN([C@@H]2CN3c4cc(Cl)nc(C(F)F)c4CC(F)(F)C[C@@H]23)CC1] | Benzyl 4-((7aS,8R)-2-chloro-4- (difluoromethyl)-6,6-difluoro- 5,6,7,7a,8,9-hexahydroazeto[1,2- a]pyrido[3,4-f]azepin-8-yl)piperazine-1- carboxylate |
| E10′ | 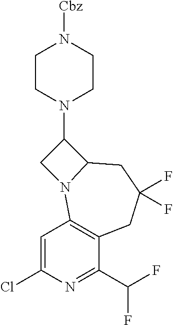 [SMILES: O=C(OCc1ccccc1)N1CCN(C2CN3c4cc(Cl)nc(C(F)F)c4CC(F)(F)CC23)CC1] | benzyl 4-(2-chloro-4-(difluoromethyl)- 6,6-difluoro-5,6,7,7a,8,9- hexahydroazeto[1,2-a]pyrido[3,4- f]azepin-8-yl)piperazine-1-carboxylate |
| E10-1 | 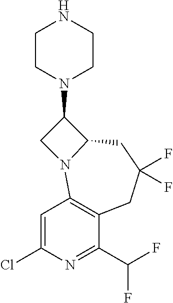 [SMILES: FC(F)c1nc(Cl)cc2c1CC(F)(F)C[C@H]1[C@H](N3CCNCC3)CN21] | (7aS,8R)-2-chloro-4-(difluoromethyl)- 6,6-difluoro-8-(piperazin-1-yl)- 5,6,7,7a,8,9-hexahydroazeto[1,2- a]pyrido[3,4-f]azepine |
| E10-1′ | 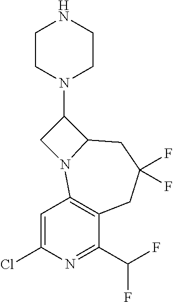 [SMILES: FC(F)c1nc(Cl)cc2c1CC(F)(F)CC1C(N3CCNCC3)CN21] | 2-chloro-4-(difluoromethyl)-6,6-difluoro- 8-(piperazin-1-yl)-5,6,7,7a,8,9- hexahydroazeto[1,2-a]pyrido[3,4- f]azepine |
| E11 | 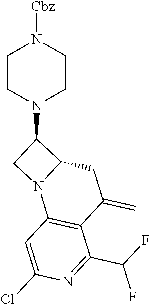 [SMILES: C=C1C[C@H]2[C@H](N3CCN(C(=O)OCc4ccccc4)CC3)CN2c2cc(Cl)nc(C(F)F)c21] | Benzyl 4-((6aS,7R)-2-chloro-4- (difluoromethyl)-5-methylene-6,6a,7,8- tetrahydro-5H-azeto[1,2- a][1,6]naphthyridin-7-yl)piperazine-1- carboxylate |
| E11′ | 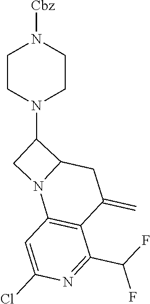 [SMILES: C=C1CC2C(N3CCN(C(=O)OCc4ccccc4)CC3)CN2c2cc(Cl)nc(C(F)F)c21] | benzyl 4-(2-chloro-4-(difluoromethyl)-5- methylene-6,6a,7,8-tetrahydro-5H- azeto[1,2-a][1,6]naphthyridin-7- yl)piperazine-1-carboxylate |
| E11-1 | 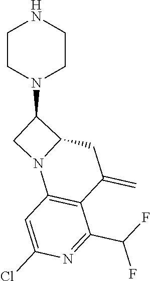 [SMILES: C=C1C[C@H]2[C@H](N3CCNCC3)CN2c2cc(Cl)nc(C(F)F)c21] | (6aS,7R)-2-chloro-4-(difluoromethyl)-5- methylene-7-(piperazin-1-yl)-6,6a,7,8- tetrahydro-5H-azeto[1,2- a][1,6]naphthyridine |
| E11-1′ | 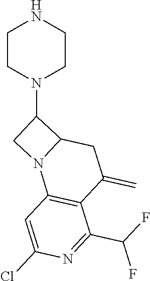 [SMILES: C=C1CC2C(N3CCNCC3)CN2c2cc(Cl)nc(C(F)F)c21] | 2-chloro-4-(difluoromethyl)-5-methylene- 7-(piperazin-1-yl)-6,6a,7,8-tetrahydro- 5H-azeto[1,2-a][1,6]naphthyridine |
| E12 | 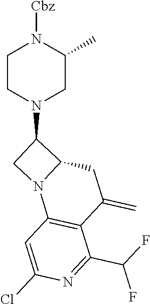 [SMILES: C=C1C[C@H]2[C@H](N3CCN(C(=O)OCc4ccccc4)[C@H](C)C3)CN2c2cc(Cl)nc(C(F)F)c21] | benzyl (R)-4-((6aS,7R)-2-chloro-4- (difluoromethyl)-5-methylene-6,6a,7,8- tetrahydro-5H-azeto[1,2- a][1,6]naphthyridin-7-yl)-2- methylpiperazine-1-carboxylate |
| E12′ | 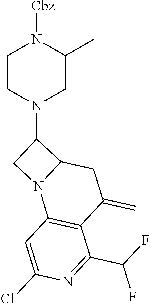 [SMILES: C=C1CC2C(N3CCN(C(=O)OCc4ccccc4)C(C)C3)CN2c2cc(Cl)nc(C(F)F)c21] | benzyl 4-(2-chloro-4-(difluoromethyl)-5- methylene-6,6a,7,8-tetrahydro-5H- azeto[1,2-a][1,6]naphthyridin-7-yl)-2- methylpiperazine-1-carboxylate |
| E12-1 | 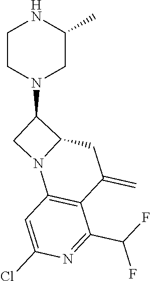 [SMILES: C=C1C[C@H]2[C@H](N3CCN[C@H](C)C3)CN2c2cc(Cl)nc(C(F)F)c21] | (6aS,7R)-2-chloro-4-(difluoromethyl)-5- methylene-7-((R)-3-methylpiperazin-1- yl)-6,6a,7,8-tetrahydro-5H-azeto[1,2- a][1,6]naphthyridine |
| E12-1′ | 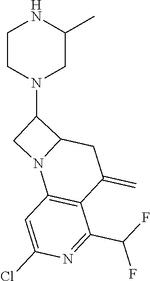 [SMILES: C=C1CC2C(N3CCNC(C)C3)CN2c2cc(Cl)nc(C(F)F)c21] | 2-chloro-4-(difluoromethyl)-5-methylene- 7-(3-methylpiperazin-1-yl)-6,6a,7,8- tetrahydro-5H-azeto[1,2- a][1,6]naphthyridine |
| E13 | 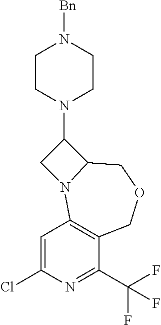 [SMILES: FC(F)(F)c1nc(Cl)cc2c1COCC1C(N3CCN(Cc4ccccc4)CC3)CN21] | 8-(4-Benzylpiperazin-1-yl)-2-chloro-4- (trifluoromethyl)-7,7a,8,9-tetrahydro-5H- azeto[2,1-c]pyrido[4,3-e][1,4]oxazepine |
| E13-1 | 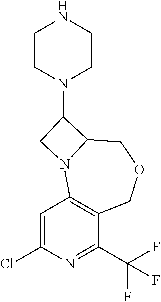 [SMILES: FC(F)(F)c1nc(Cl)cc2c1COCC1C(N3CCNCC3)CN21] | 2-chloro-8-(piperazin-1-yl)-4- (trifluoromethyl)-7,7a,8,9-tetrahydro-5H- azeto[2,1-c]pyrido[4,3-e][1,4]oxazepine |

Also provided herein are intermediates listed in Table INT-F, and pharmaceutically acceptable salts thereof.

TABLE INT-F

| Intermediate | | |
|---|---|---|
| Ref. | Structure | Name |
| F1 | 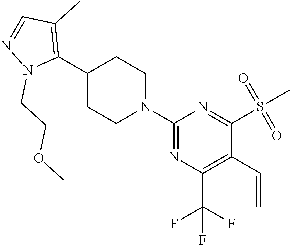 [SMILES: C=Cc1c(C(F)(F)F)nc(N2CCC(c3c(C)cnn3CCOC)CC2)nc1S(C)(=O)=O] | 2-(4-(1-(2-Methoxyethyl)-4-methyl-1H- pyrazol-5-yl)piperidin-1-yl)-4- (methylsulfonyl)-6-(trifluoromethyl)-5- vinylpyrimidine |
| F2 | 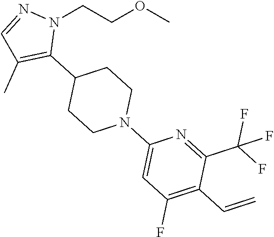 [SMILES: C=Cc1c(F)cc(N2CCC(c3c(C)cnn3CCOC)CC2)nc1C(F)(F)F] | 4-fluoro-6-(4-(1-(2-methoxyethyl)-4- methyl-1H-pyrazol-5-yl)piperidin-1-yl)-2- (trifluoromethyl)-3-vinylpyridine |
| F3 | 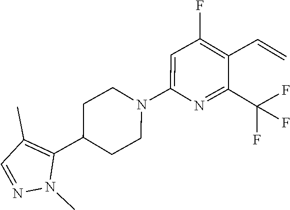 [SMILES: C=Cc1c(F)cc(N2CCC(c3c(C)cnn3C)CC2)nc1C(F)(F)F] | 6-(4-(1,4-dimethyl-1H-pyrazol-5- yl)piperidin-1-yl)-4-fluoro-2- (trifluoromethyl)-3-vinylpyridine |
| F4 | 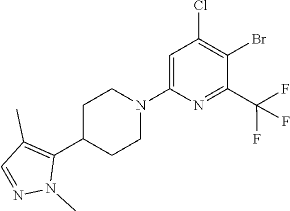 [SMILES: Cc1cnn(C)c1C1CCN(c2cc(Cl)c(Br)c(C(F)(F)F)n2)CC1] | 3-bromo-4-chloro-6-(4-(1,4-dimethyl-1H- pyrazol-5-yl)piperidin-1-yl)-2- (trifluoromethyl)pyridine |

Also provided herein are intermediates listed in Table INT, and pharmaceutically acceptable salts thereof.

TABLE INT

| Intermediate | | |
|---|---|---|
| Ref. | Structure | Name |
| 1.2 | 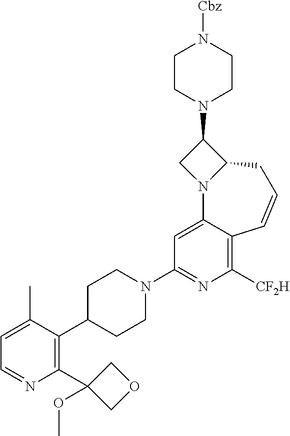 [SMILES: COC1(c2nccc(C)c2C2CCN(c3cc4c(c(C(F)F)n3)C=CC[C@H]3[C@H](N5CCN(C(=O)OCc6ccccc6)CC5)CN43)CC2)COC1] | benzyl 4-((7aS,8R)-4-(difluoromethyl)-2-(4-(2- (3-methoxyoxetan-3-yl)-4-methylpyridin-3- yl)piperidin-1-yl)-7,7a,8,9-tetrahydroazeto[1,2- a]pyrido[3,4-f]azepin-8-yl)piperazine-1- carboxylate |
| 1.3 | 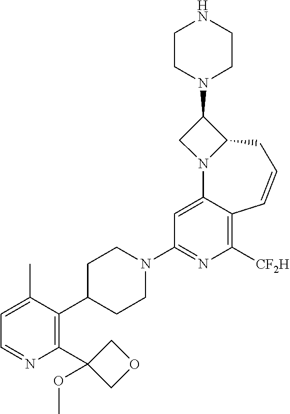 [SMILES: COC1(c2nccc(C)c2C2CCN(c3cc4c(c(C(F)F)n3)C=CC[C@H]3[C@H](N5CCNCC5)CN43)CC2)COC1] | (7aS,8R)-4-(difluoromethyl)-2-(4-(2-(3- methoxyoxetan-3-yl)-4-methylpyridin-3- yl)piperidin-1-yl)-8-(piperazin-1-yl)- 7,7a,8,9-tetrahydroazeto[1,2- a]pyrido[3,4-f]azepine |
| 2.2 | 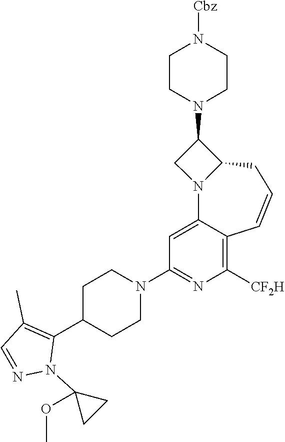 [SMILES: COC1(n2ncc(C)c2C2CCN(c3cc4c(c(C(F)F)n3)C=CC[C@H]3[C@H](N5CCN(C(=O)OCc6ccccc6)CC5)CN43)CC2)CC1] | benzyl 4-((7aS,8R)-4-(difluoromethyl)-2-(4-(1- (1-methoxycyclopropyl)-4-methyl-1H-pyrazol- 5-yl)piperidin-1-yl)-7,7a,8,9-tetrahydroazeto[1,2- a]pyrido[3,4-f]azepin-8-yl)piperazine-1- carboxylate |
| 2.3 | 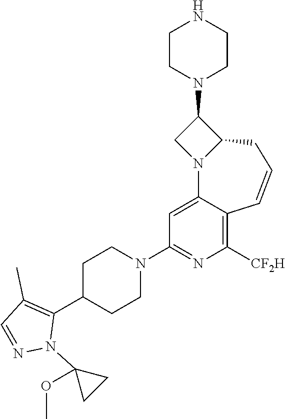 [SMILES: COC1(n2ncc(C)c2C2CCN(c3cc4c(c(C(F)F)n3)C=CC[C@H]3[C@H](N5CCNCC5)CN43)CC2)CC1] | (7aS,8R)-4-(difluoromethyl)-2-(4-(1-(1- methoxycyclopropyl)-4-methyl-1H-pyrazol-5- yl)piperidin-1-yl)-8-(piperazin-1-yl)- 7,7a,8,9-tetrahydroazeto[1,2- a]pyrido[3,4-f]azepine |
| 3.2 | 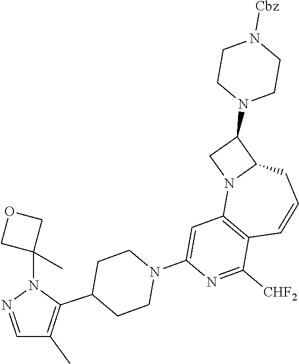 [SMILES: Cc1cnn(C2(C)COC2)c1C1CCN(c2cc3c(c(C(F)F)n2)C=CC[C@H]2[C@H](N4CCN(C(=O)OCc5ccccc5)CC4)CN32)CC1] | benzyl 4-((7aS,8R)-4-(difluoromethyl)-2-(4-(4- methyl-1-(3-methyloxetan-3-yl)-1H-pyrazol-5- yl)piperidin-1-yl)-7,7a,8,9-tetrahydroazeto[1,2- a]pyrido[3,4-f]azepin-8-yl)piperazine-1- carboxylate |
| 3.3 | 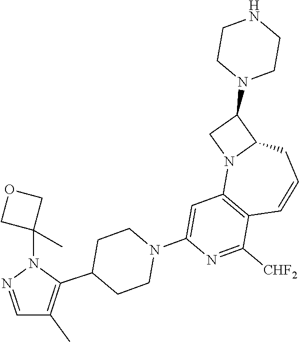 [SMILES: Cc1cnn(C2(C)COC2)c1C1CCN(c2cc3c(c(C(F)F)n2)C=CC[C@H]2[C@H](N4CCNCC4)CN32)CC1] | (7aS,8R)-4-(difluoromethyl)-2-(4-(4-methyl-1- (3-methyloxetan-3-yl)-1H-pyrazol-5- yl)piperidin-1-yl)-8-(piperazin-1-yl)- 7,7a,8,9-tetrahydroazeto[1,2- a]pyrido[3,4-f]azepine |
| 17.1 | 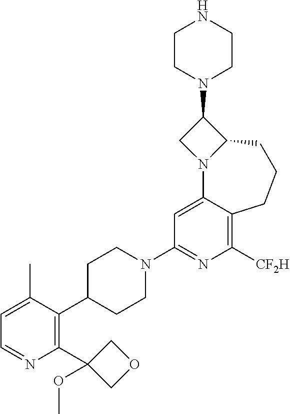 [SMILES: COC1(c2nccc(C)c2C2CCN(c3cc4c(c(C(F)F)n3)CCC[C@H]3[C@H](N5CCNCC5)CN43)CC2)COC1] | (7aS,8R)-4-(difluoromethyl)-2-(4-(2-(3- methoxyoxetan-3-yl)-4-methylpyridin-3- yl)piperidin-1-yl)-8-(piperazin-1-yl)- 5,6,7,7a,8,9-hexahydroazeto[1,2-a]pyrido[3,4- f]azepine |
| 18.1 | 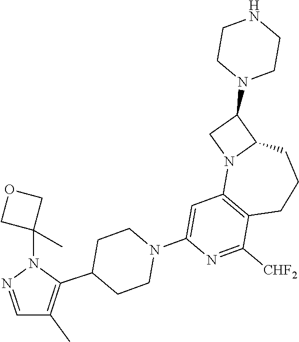 [SMILES: Cc1cnn(C2(C)COC2)c1C1CCN(c2cc3c(c(C(F)F)n2)CCC[C@H]2[C@H](N4CCNCC4)CN32)CC1] | (7aS,8R)-4-(difluoromethyl)-2-(4-(4-methyl-1- (3-methyloxetan-3-yl)-1H-pyrazol-5- yl)piperidin-1-yl)-8-(piperazin-1-yl)- 5,6,7,7a,8,9-hexahydroazeto[1,2-a]pyrido[3,4- f]azepine |
| 19.2 | 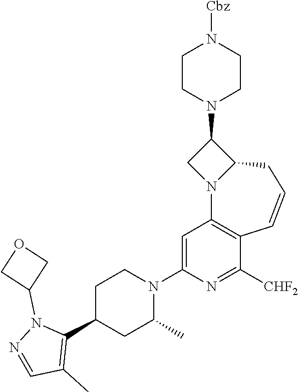 [SMILES: Cc1cnn(C2COC2)c1[C@H]1CCN(c2cc3c(c(C(F)F)n2)C=CC[C@H]2[C@H](N4CCN(C(=O)OCc5ccccc5)CC4)CN32)[C@H](C)C1] | benzyl 4-((7aS,8R)-4-(difluoromethyl)-2- ((2R,4S)-2-methyl-4-(4-methyl-1-(oxetan-3- yl)-1H-pyrazol-5-yl)piperidin-1-yl)- 7,7a,8,9-tetrahydroazeto[1,2- a]pyrido[3,4-f]azepin-8-yl)piperazine-1- carboxylate |
| 19.3 | 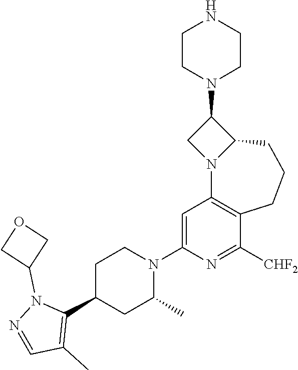 [SMILES: Cc1cnn(C2COC2)c1[C@H]1CCN(c2cc3c(c(C(F)F)n2)CCC[C@H]2[C@H](N4CCNCC4)CN32)[C@H](C)C1] | (7aS,8R)-4-(difluoromethyl)-2-((2R,4S)-2- methyl-4-(4-methyl-1-(oxetan-3-yl)-1H- pyrazol-5-yl)piperidin-1-yl)-8-(piperazin-1-yl)- 5,6,7,7a,8,9-hexahydroazeto[1,2-a]pyrido[3,4- f]azepine |
| 20.2 | 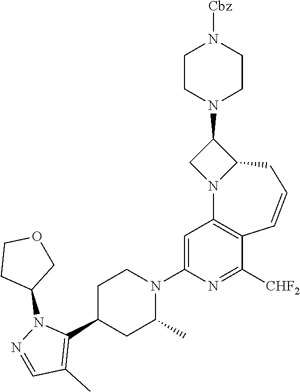 [SMILES: Cc1cnn([C@H]2CCOC2)c1[C@H]1CCN(c2cc3c(c(C(F)F)n2)C=CC[C@H]2[C@H](N4CCN(C(=O)OCc5ccccc5)CC4)CN32)[C@H](C)C1] | benzyl 4-((7aS,8R)-4-(difluoromethyl)-2- ((2R,4S)-2-methyl-4-(4-methyl-1-((S)- tetrahydrofuran-3-yl)-1H-pyrazol-5- yl)piperidin-1-yl)-7,7a,8,9-tetrahydroazeto[1,2- a]pyrido[3,4-f]azepin-8-yl)piperazine-1- carboxylate |
| 20.3 | 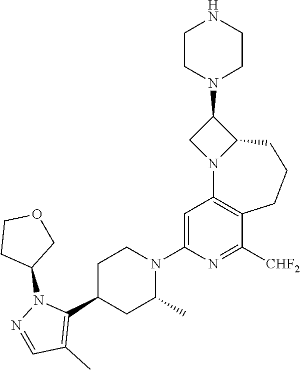 [SMILES: Cc1cnn([C@H]2CCOC2)c1[C@H]1CCN(c2cc3c(c(C(F)F)n2)CCC[C@H]2[C@H](N4CCNCC4)CN32)[C@H](C)C1] | (7aS,8R)-4-(difluoromethyl)-2-((2R,4S)-2- methyl-4-(4-methyl-1-((S)-tetrahydrofuran-3- yl)-1H-pyrazol-5-yl)piperidin-1-yl)-8- (piperazin-1-yl)-5,6,7,7a,8,9- hexahydroazeto[1,2-a]pyrido[3,4-f]azepine |
| 21.2 | 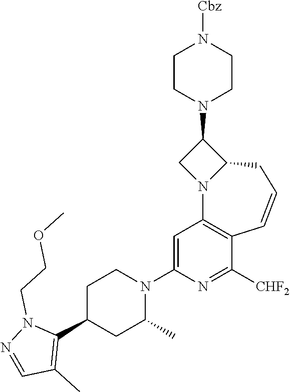 [SMILES: COCCn1ncc(C)c1[C@H]1CCN(c2cc3c(c(C(F)F)n2)C=CC[C@H]2[C@H](N4CCN(C(=O)OCc5ccccc5)CC4)CN32)[C@H](C)C1] | benzyl 4-((7aS,8R)-4-(difluoromethyl)-2- ((2R,4S)-4-(1-(2-methoxyethyl)-4-methyl-1H- pyrazol-5-yl)-2-methylpiperidin-1-yl)- 7,7a,8,9-tetrahydroazeto[1,2- a]pyrido[3,4-f]azepin-8-yl)piperazine-1- carboxylate |
| 21.3 | 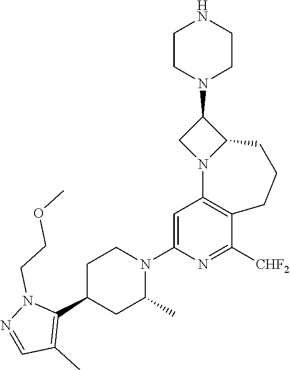 [SMILES: COCCn1ncc(C)c1[C@H]1CCN(c2cc3c(c(C(F)F)n2)CCC[C@H]2[C@H](N4CCNCC4)CN32)[C@H](C)C1] | (7aS,8R)-4-(difluoromethyl)-2-((2R,4S)-4-(1- (2-methoxyethyl)-4-methyl-1H-pyrazol-5-yl)- 2-methylpiperidin-1-yl)-8-(piperazin-1-yl)- 5,6,7,7a,8,9-hexahydroazeto[1,2-a]pyrido[3,4- f]azepine |
| 67.2 | 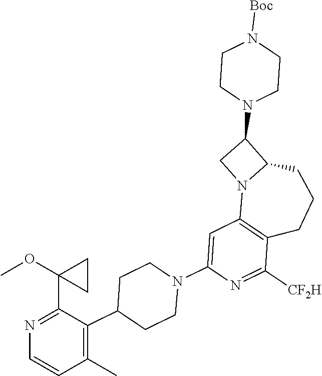 [SMILES: COC1(c2nccc(C)c2C2CCN(c3cc4c(c(C(F)F)n3)CCC[C@H]3[C@H](N5CCN(C(=O)OC(C)(C)C)CC5)CN43)CC2)CC1] | tert-butyl 4-((7aS,8aS)-4-(difluoromethyl)-2-(4- (2-(1-methoxycyclopropyl)-4-methylpyridin-3- yl)piperidin-1-yl)-5,6,7,7a,8,9- hexahydroazeto[1,2-a]pyrido[3,4-f]azepin-8- yl)piperazine-1-carboxylate |
| 67.3 | 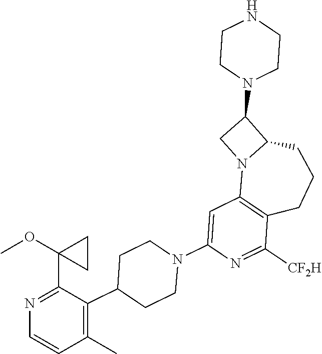 [SMILES: COC1(c2nccc(C)c2C2CCN(c3cc4c(c(C(F)F)n3)CCC[C@H]3[C@H](N5CCNCC5)CN43)CC2)CC1] | (7aS,8R)-4-(difluoromethyl)-2-(4-(2-(1- methoxycyclopropyl)-4-methylpyridin-3- yl)piperidin-1-yl)-8-(piperazin-1-yl)- 5,6,7,7a,8,9-hexahydroazeto[1,2-a]pyrido[3,4- f]azepine |
| 75.1 | 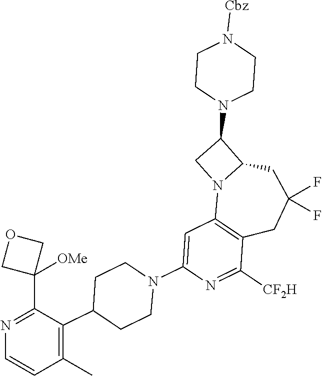 [SMILES: COC1(c2nccc(C)c2C2CCN(c3cc4c(c(C(F)F)n3)CC(F)(F)C[C@H]3[C@H](N5CCN(C(=O)OCc6ccccc6)CC5)CN43)CC2)COC1] | benzyl 4-((7aS,8R)-4-(difluoromethyl)-6,6- difluoro-2-(4-(2-(3-methoxyoxetan-3-yl)-4- methylpyridin-3-yl)piperidin-1-yl)- 5,6,7,7a,8,9-hexahydroazeto[1,2-a]pyrido[3,4- f]azepin-8-yl)piperazine-1-carboxylate |
| 75.2 | 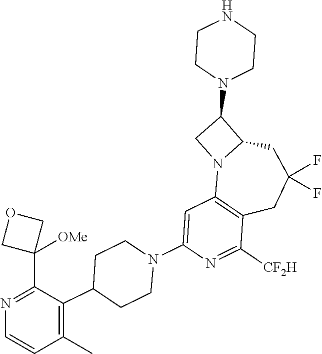 [SMILES: COC1(c2nccc(C)c2C2CCN(c3cc4c(c(C(F)F)n3)CC(F)(F)C[C@H]3[C@H](N5CCNCC5)CN43)CC2)COC1] | (7aS,8R)-4-(difluoromethyl)-6,6-difluoro-2-(4- (2-(3-methoxyoxetan-3-yl)-4-methylpyridin-3- yl)piperidin-1-yl)-8-(piperazin-1-yl)- 5,6,7,7a,8,9-hexahydroazeto[1,2-a]pyrido[3,4- f]azepine |
| 86.1 | 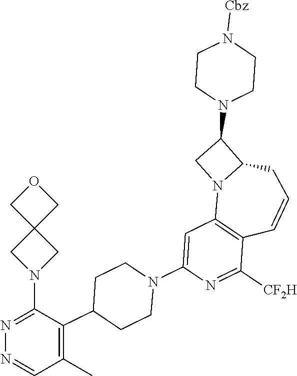 [SMILES: Cc1cnnc(N2CC3(COC3)C2)c1C1CCN(c2cc3c(c(C(F)F)n2)C=CC[C@H]2[C@H](N4CCN(C(=O)OCc5ccccc5)CC4)CN32)CC1] | benzo 4-((7aS,8R)-4-(difluoromethyl)-2-(4-(5- methyl-3-(2-oxa-6-azaspiro[3.3]heptan-6- yl)pyridazin-4-yl)piperidin-1-yl)-7,7a,8,9- tetrahydroazeto[1,2-a]pyrido[3,4-f]azepin-8- yl)piperazine-1-carboxylate |
| 86.2 | 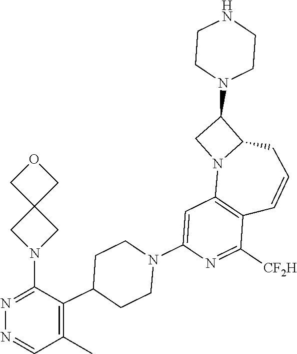 [SMILES: Cc1cnnc(N2CC3(COC3)C2)c1C1CCN(c2cc3c(c(C(F)F)n2)C=CC[C@H]2[C@H](N4CCNCC4)CN32)CC1] | 6-(4-(1-((7aS,8R)-4-(difluoromethyl)-8- (piperazin-1-yl)-7,7a,8,9-tetrahydroazeto[1,2- a]pyrido[3,4-f]azepin-2-yl)piperidin-4-yl)-5- methylpyridazin-3-yl)-2-oxa-6- azaspiro[3.3]heptane |
| 97.1 | 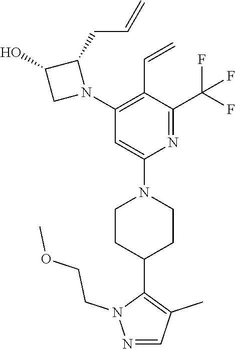 [SMILES: C=CC[C@H]1[C@@H](O)CN1c1cc(N2CCC(c3c(C)cnn3CCOC)CC2)nc(C(F)(F)F)c1C=C] | (2S,3S)-2-allyl-1-(6-(4-(1-(2-methoxyethyl)-4- methyl-1H-pyrazol-5-yl)piperidin-1-yl)-2- (trifluoromethyl)-3-vinylpyridin-4-yl)azetidin- 3-ol |
| 97.2 | 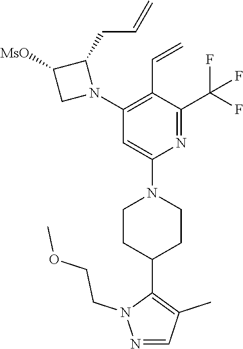 [SMILES: C=CC[C@H]1[C@@H](OS(C)(=O)=O)CN1c1cc(N2CCC(c3c(C)cnn3CCOC)CC2)nc(C(F)(F)F)c1C=C] | (2S,3S)-2-allyl-1-(6-(4-(1-(2-methoxyethyl)-4- methyl-1H-pyrazol-5-yl)piperidin-1-yl)-2- (trifluoromethyl)-3-vinylpyridin-4-yl)azetidin- 3-yl methanesulfonamide |
| 97.3 | 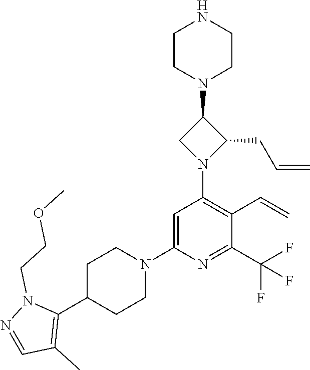 [SMILES: C=CC[C@H]1[C@H](N2CCNCC2)CN1c1cc(N2CCC(c3c(C)cnn3CCOC)CC2)nc(C(F)(F)F)c1C=C] | 1-((2S,3R)-2-allyl-1-(6-(4-(1-(2- methoxyethyl)-4-methyl-1H-pyrazol-5- yl)piperidin-1-yl)-2-(trifluoromethyl)-3- vinylpyridin-4-yl)azetidin-3-yl)piperazine |
| 97.4 | 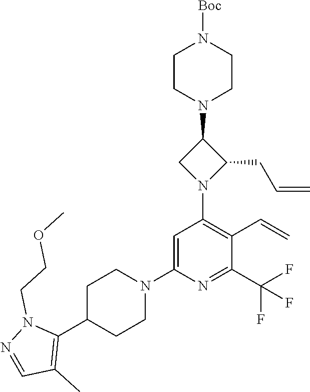 [SMILES: C=CC[C@H]1[C@H](N2CCN(C(=O)OC(C)(C)C)CC2)CN1c1cc(N2CCC(c3c(C)cnn3CCOC)CC2)nc(C(F)(F)F)c1C=C] | tert-butyl 4-((2S,3R)-2-allyl-1-(6-(4-(1-(2- methoxyethyl)-4-methyl-1H-pyrazol-5- yl)piperidin-1-yl)-2-(trifluoromethyl)-3- vinylpyridin-4-yl)azetidin-3-yl)piperazine-1- carboxylate |
| 97.5 | 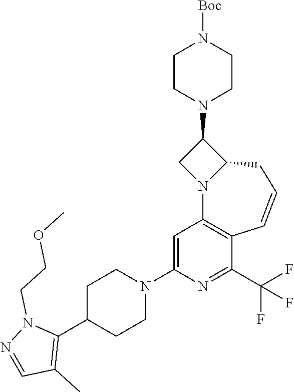 [SMILES: COCCn1ncc(C)c1C1CCN(c2cc3c(c(C(F)(F)F)n2)C=CC[C@H]2[C@H](N4CCN(C(=O)OC(C)(C)C)CC4)CN32)CC1] | tert-butyl 4-((7aS,8R)-2-(4-(1-(2- methoxyethyl)-4-methyl-1H-pyrazol-5- yl)piperidin-1-yl)-4-(trifluoromethyl)-7,7a,8,9- tetrahydroazeto[1,2-a]pyrido[3,4-f]azepin-8- yl)piperazine-1-carboxylate |
| 97.6 | 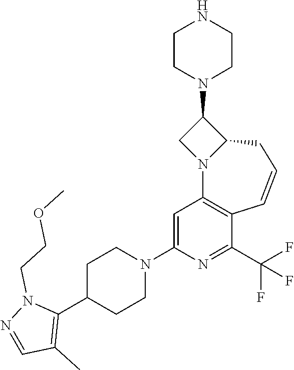 [SMILES: COCCn1ncc(C)c1C1CCN(c2cc3c(c(C(F)(F)F)n2)C=CC[C@H]2[C@H](N4CCNCC4)CN32)CC1] | (7aS,8R)-2-(4-(1-(2-methoxyethyl)-4-methyl- 1H-pyrazol-5-yl)piperidin-1-yl)-8-(piperazin- 1-yl)-4-(trifluoromethyl)- 7,7a,8,9-tetrahydroazeto[1,2- a]pyrido[3,4-f]azepine |
| 101.1 | 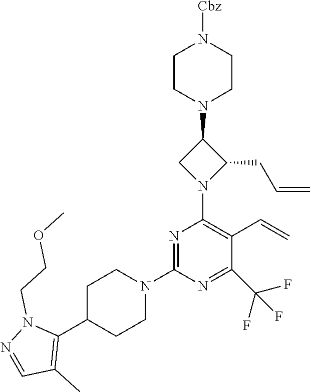 [SMILES: C=CC[C@H]1[C@H](N2CCN(C(=O)OCc3ccccc3)CC2)CN1c1nc(N2CCC(c3c(C)cnn3CCOC)CC2)nc(C(F)(F)F)c1C=C] | benzyl 4-((2S,3R)-2-allyl-1-(2-(4-(1-(2- methoxyethyl)-4-methyl-1H-pyrazol-5- yl)piperidin-1-yl)-6-(trifluoromethyl)-5- vinylpyrimidin-4-yl)azetidin-3-yl)piperazine-1- carboxylate |
| 101.2 | 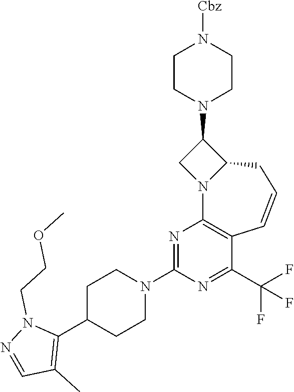 [SMILES: COCCn1ncc(C)c1C1CCN(c2nc3c(c(C(F)(F)F)n2)C=CC[C@H]2[C@H](N4CCN(C(=O)OCc5ccccc5)CC4)CN32)CC1] | benzyl 4-((7aS,8R)-2-(4-(1-(2-methoxyethyl)- 4-methyl-1H-pyrazol-5-yl)piperidin-1-yl)-4- (trifluoromethyl)-7,7a,8,9-tetrahydroazeto[1,2- a]pyrimido[5,4-f]azepin-8-yl)piperazine-1- carboxylate |
| 101.3 | 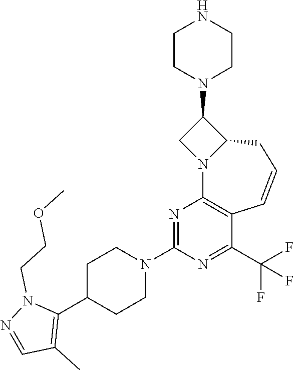 [SMILES: COCCn1ncc(C)c1C1CCN(c2nc3c(c(C(F)(F)F)n2)C=CC[C@H]2[C@H](N4CCNCC4)CN32)CC1] | (7aS,8R)-2-(4-(1-(2-methoxyethyl)-4-methyl- 1H-pyrazol-5-yl)piperidin-1-yl)-8-(piperazin- 1-yl)-4-(trifluoromethyl)- 7,7a,8,9-tetrahydroazeto[1,2- a]pyrimido[5,4-f]azepine |

Another aspect of the disclosure is a process for preparing a compound described herein (e.g., a compound of Formula (I), Formula (I′), Formula (IA), Formula (IB), Formula (IE), Formula (IF), Formula (IG), Formula (II), Formula (II′), Formula (IIA), Formula (IIB), Formula (IIC), Formula (IID), Formula (IIE), and Formula (IIF), or a compound listed in Table A, Table A′, Table B, Table B′, and Table E), or a pharmaceutically acceptable salt of any of the foregoing comprising converting an intermediate described herein, such as an intermediate of Formula (Int-AA), Formula (Int-AB), Formula (Int-AC), Formula (Int-AD), Formula (Int-AE), Formula (Int-AF), Formula (Int-AG), Formula (Int-AH), Formula (Int-AI), Formula (Int-AJ), Formula (Int-B), Formula (Int-C), Formula (Int-D), and Formula (Int-E), or an intermediate listed in Table INT-A, Table INT-A′, Table INT-B, Table INT-C, Table INT-D, Table INT-E, Table INT-F, or Table INT, a nitrogen-protected analog thereof, or a pharmaceutically acceptable salt thereof, into a compound or salt of the disclosure (e.g., a compound of Formula (I), Formula (I′), Formula (IA), Formula (IB), Formula (IE), Formula (IF), Formula (IG), Formula (II), Formula (II′), Formula (IIA), Formula (IIB), Formula (IIC), Formula (IID), Formula (IIE), and Formula (IIF), or a compound listed in Table A, Table A′, Table B, Table B′, and Table E), or a pharmaceutically acceptable salt of any of the foregoing. In some cases, the intermediate is a compound of Formula (Int-AA), Formula (Int-AB), Formula (Int-AC), Formula (Int-AD), Formula (Int-AE), Formula (Int-AF), Formula (Int-AG), Formula (Int-AH), Formula (Int-AI), Formula (Int-AJ) or a compound listed in Table A or Table A′, a nitrogen-protected analog of any of the foregoing, or a pharmaceutically acceptable salt of any of the foregoing. In some cases, the intermediate is a compound of Formula (Int-AA), a nitrogen-protected analog thereof, or a pharmaceutically acceptable salt of any of the foregoing. In some cases, the intermediate is a compound of Formula (Int-AB), a nitrogen-protected analog thereof, or a pharmaceutically acceptable salt of any of the foregoing. In some cases, the intermediate is a compound of Formula (Int-AC), a nitrogen-protected analog thereof, or a pharmaceutically acceptable salt of any of the foregoing. In some cases, the intermediate is a compound of Formula (Int-AD), a nitrogen-protected analog thereof, or a pharmaceutically acceptable salt of any of the foregoing. In some cases, the intermediate is a compound of Formula (Int-AE), a nitrogen-protected analog thereof, or a pharmaceutically acceptable salt of any of the foregoing. In some cases, the intermediate is a compound of Formula (Int-AF), a nitrogen-protected analog thereof, or a pharmaceutically acceptable salt of any of the foregoing. In some cases, the intermediate is a compound of Formula (Int-AG), a nitrogen-protected analog thereof, or a pharmaceutically acceptable salt of any of the foregoing. In some cases, the intermediate is a compound of Formula (Int-AH), a nitrogen-protected analog thereof, or a pharmaceutically acceptable salt of any of the foregoing. In some cases, the intermediate is a compound of Formula (Int-AI), a nitrogen-protected analog thereof, or a pharmaceutically acceptable salt of any of the foregoing. In some cases, the intermediate is a compound of Formula (Int-AJ), a nitrogen-protected analog thereof, or a pharmaceutically acceptable salt of any of the foregoing. In some cases, the intermediate is a compound listed in Table INT-A or Table INT-A′, a nitrogen-protected analog thereof, or a pharmaceutically acceptable salt of any of the foregoing. In some cases, the intermediate is a compound of Formula (Int-B), a nitrogen-protected analog thereof, or a pharmaceutically acceptable salt of any of the foregoing. In some cases, the intermediate is a compound listed in Table INT-B, a nitrogen-protected analog thereof, or a pharmaceutically acceptable salt of any of the foregoing. In some cases, the intermediate is a compound of Formula (Int-C), a nitrogen-protected analog thereof, or a pharmaceutically acceptable salt of any of the foregoing. In some cases, the intermediate is a compound listed in Table INT-C, a nitrogen-protected analog thereof, or a pharmaceutically acceptable salt of any of the foregoing. In some cases, the intermediate is a compound of Formula (Int-D), a nitrogen-protected analog thereof, or a pharmaceutically acceptable salt of any of the foregoing. In some cases, the intermediate is a compound listed in Table INT-D, a nitrogen-protected analog thereof, or a pharmaceutically acceptable salt of any of the foregoing. In some cases, the intermediate is a compound of Formula (Int-E), a nitrogen-protected analog thereof, or a pharmaceutically acceptable salt of any of the foregoing. In some cases, the intermediate is a compound listed in Table INT-E, a nitrogen-protected analog thereof, or a pharmaceutically acceptable salt of any of the foregoing. In some cases, the intermediate is a compound listed in Table INT-F, a nitrogen-protected analog thereof, or a pharmaceutically acceptable salt of any of the foregoing. In some cases, the intermediate is a compound listed in Table INT, a nitrogen-protected analog thereof, or a pharmaceutically acceptable salt of any of the foregoing.

### OTHER EMBODIMENTS

Provided herein as Embodiment 1 is a compound of Formula (I):

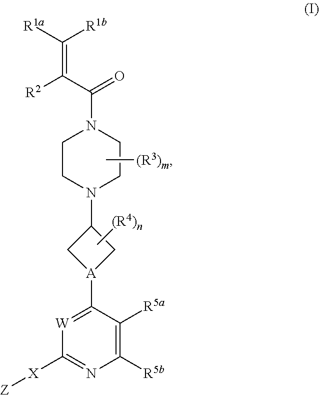

or a pharmaceutically acceptable salt thereof,
wherein:
m is 0, 1, 2, 3, or 4;
n is 0, 1, or 2;
o is 0, 1, 2, 3, or 4;
A is N, CH, C-halo, C—CN, C—C₁₋₃alkyl, C₁₋₃haloalkyl, C—C₀₋₂alkyleneOH, or C—C₀₋₃alkylene-C₁₋₄alkoxy:
W is CH, C-halo, C—CN, C—C₁₋₃alkyl, C—C₁₋₃haloalkyl, C—C₀₋₂alkyleneOH, or C—C₀₋₃alkylene-C₁₋₄alkoxy;
X is

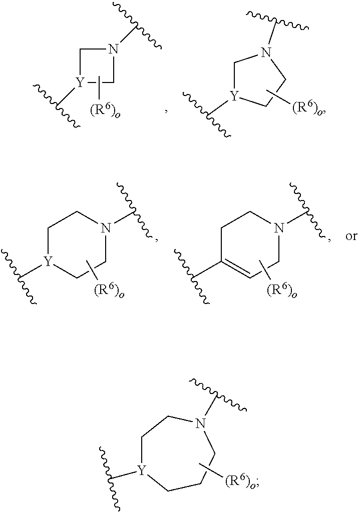

Y is N, C—H, C-halo, C—CN, C—C₁₋₃alkyl, C—C₁₋₃haloalkyl, C—C₀₋₂alkyleneOH, or C—C₀₋₃alkylene-C₁₋₄alkoxy;
Z is phenyl, heteroaryl comprising 5 or 6 total ring atoms and 1-3 heteroatoms selected from N, O, and S, or a bicyclic ring comprising a heteroaryl ring having 5 or 6 total ring atoms and 1-3 heteroatoms selected from N, O, and S fused to cycloalkyl ring having 5 or 6 total ring atoms or a heterocycloalkyl ring having 5 or 6 total ring atoms and 1 or 2 heteroatoms selected from N, O, and S; wherein each of the phenyl, heteroaryl, and bicyclic rings is optionally substituted with 1-4 substituents;
each of R^{1a}, R^{1b}, and R² independently is H, D, halo, C₁₋₄alkyl, C₁₋₄haloalkyl, C₁₋₂alkylene-OH, C₀₋₂alkylene-C₁₋₄alkoxy, C₀₋₂alkylene-C₁₋₄haloalkoxy, C₀₋₂alkylene-CN, C₀₋₂alkylene-N(R^{N1})₂, C₁₋₂alkylene-heterocycloalkyl having 3-6 total ring atoms and 1 or 2 heteroatoms selected from N, O, and S, or R^{1b} and R², together with the carbon atoms to which they are attached, from a group;

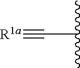

each R³ independently is C₁₋₃alkyl, C₁₋₃haloalkyl, C₀₋₃alkyleneCN, C₀₋₂alkyleneOH, C₀₋₃alkylene-C₁₋₃alkoxy, oxo, spiro-cycloalkyl having 3-7 total ring atoms, spiro-heterocycloalkyl having 3-7 total ring atoms and 1 or 2 heteroatoms selected from N, O and S, or two adjacent R³, together with the atoms to which they are attached, form a fused cycloalkyl ring having 3-7 total ring atoms;
each R⁴ independently is C₁₋₃alkyl, C₁₋₄haloalkyl, C₀₋₃alkyleneCN, C₁₋₃alkyleneOH, C₁₋₃alkylene-C₁₋₃alkoxy, oxo, spiro-cycloalkyl having 3-7 total ring atoms, or spiro-heterocycloalkyl having 4-7 total ring atoms and 1 or 2 heteroatoms selected from N, O and S;
R^{5a} is H, CN, halo, C₁₋₃alkyl, C₁₋₃haloalkyl, C₂₋₆alkenyl, C₂₋₃alkynyl, C₀₋₃alkyleneOH, C₀₋₃alkylene-C₁₋₃alkoxy, or cycloalkyl having 3-5 total ring atoms; or R^{5a} and an R⁴, together with the atoms to which they are attached, form an optionally substituted ring having 6-10 total ring atoms and 0, 1, or 2 heteroatoms selected from N, O, and S, wherein the ring is saturated or unsaturated:
R^{2b} is C₁₋₄haloalkyl, C₁₋₃alkyl, C₂₋₆alkenyl, C₂₋₃alkynyl, halo, C₁₋₃alkoxy, C₁₋₃thioalkoxy, cycloalkyl having 3-7 total ring atoms, cycloalkenyl having 5-7 total ring atoms, heterocycloalkyl having 3-7 total ring atoms and 1-3 heteroatoms selected from N, O, and S, or heterocycloalkenyl having 5-7 total ring atoms and 1-3 heteroatoms selected from N, O, and S, wherein each of foregoing is independently optionally substituted with 1-3 substituents, or R^{5a} and R^{5b}, together with the atoms to which they are attached, form a cycloalkyl ring having 3-7 total ring atoms;
each R⁶ independently is halo, CN, oxo, C₁₋₃alkyl, C₁₋₃haloalkyl, C₀₋₃alkyleneOH, C₀₋₃alkylene-C₁₋₃alkoxy, deuterated C₀₋₆alkylene-C₁₋₃alkoxy, C₁₋₄alkylene-N(R^{N1})₂, spiro-cycloalkyl having 3-7 total ring atoms, spiro-heterocycloalkyl having 4-7 total ring atoms and 1 or 2 heteroatoms selected from N, O and S; or two adjacent R⁶, together with the atoms to which they are attached, form a fused cycloalkyl ring having 3-7 total ring atoms; or Y and an adjacent R⁶, together with the atoms to which they are attached, form a fused cycloalkyl ring having 3-7 total ring atoms; wherein the fused cycloalkyl ring of any of the foregoing is optionally substituted with 1 or 2 substituents; or two non-adjacent Rejoin together to form a C₁₋₃alkylene bridge or a C₁₋₃ether bridge; and
each R^{N1} independently is H or C₁₋₄alkyl.

Provided herein as Embodiment 2 is the compound or salt of Embodiment 1, wherein at least one of R^{1a}, R^{1b}, and R² is H or D.

Provided herein as Embodiment 3 is the compound or salt of Embodiment 2, wherein each of R^{1a}, R^{1b}, and R² independently is H or D.

Provided herein as Embodiment 4 is the compound or salt of Embodiment 3, wherein each of R^{1a}, R^{1b}, and R² independently is H.

Provided herein as Embodiment 5 is the compound or salt of Embodiment 3, wherein each of R^{1a}, R^{1b}, and R² independently is D.

Provided herein as Embodiment 6 is the compound or salt of Embodiment 1 or 2, wherein at least one of R^{1a}, R^{1b}, and R² is halo.

Provided herein as Embodiment 7 is the compound or salt of Embodiment 6, wherein R^{a} is halo and each of R^{1b} and R² is H.

Provided herein as Embodiment 8 is the compound or salt of Embodiment 6 or 7, wherein each halo independently is Br, Cl, or F.

Provided herein as Embodiment 9 is the compound or salt of Embodiment 1 or 2, wherein at least one of R^{1a}, R^{1b}, and R² is C₁₋₄alkyl or C₁₋₄haloalkyl.

Provided herein as Embodiment 10 is the compound or salt of Embodiment 9, wherein at least one of R^{1a}, R^{1b}, and R² is CH₃ or CF₃.

Provided herein as Embodiment 11 is the compound or salt of Embodiment 1 or 2, wherein at least one of R^{1a}, R^{1b}, and R² is C₁₋₂alkylene-OH, C₀₋₂alkylene-C; alkoxy, C₀₋₂alkylene-C₁₋₄haloalkoxy. C₀₋₂alkylene-CN, or C₀₋₂alkylene-N(R^{N1})₂.

Provided herein as Embodiment 12 is the compound or salt of Embodiment 11, wherein each R^{N1} independently is H or CH₃.

Provided herein as Embodiment 13 is the compound or salt of Embodiment 12, wherein each R^{N1} independently is H.

Provided herein as Embodiment 14 is the compound or salt of Embodiment 11 or 12, wherein at least one of R^{1a}, R^{1b}, and R² is CH₂OH, OCH₃, CH₂OCH₃, OCF₃, CH₂OCF₃, CN, CH₂CN, NH₂, N(CH₃)₂, CH₂NH₂, or CH₂N(CH₃)₂.

Provided herein as Embodiment 15 is the compound or salt of Embodiment 1 or 2, wherein at least one of R^{1a}, R^{1b}, and R² is C₁₋₂alkylene-heterocycloalkyl having 3-6 total ring atoms and 1 or 2 heteroatoms selected from N, O, and S.

Provided herein as Embodiment 16 is the compound or salt of Embodiment 15, wherein the heterocycloalkyl is aziridinyl, azetidinyl, pyrrolidinyl, piperidinyl, or morpholinyl.

Provided herein as Embodiment 17 is the compound or salt of Embodiment 16, wherein at least one of R^{1a}, R^{1b}, and R² is aziridin-1-yl-methyl, azetidin-1-yl-methyl, pyrrolidine-1-yl-methyl, piperidin-1-yl-methyl, or morpholin-1-yl-methyl.

Provided herein as Embodiment 18 is the compound or salt of Embodiment 1, wherein R^{1b} and R², together with the carbon atoms to which they are attached, from a

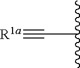

group.

Provided herein as Embodiment 19 is the compound or salt of Embodiment 1, wherein

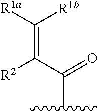

is

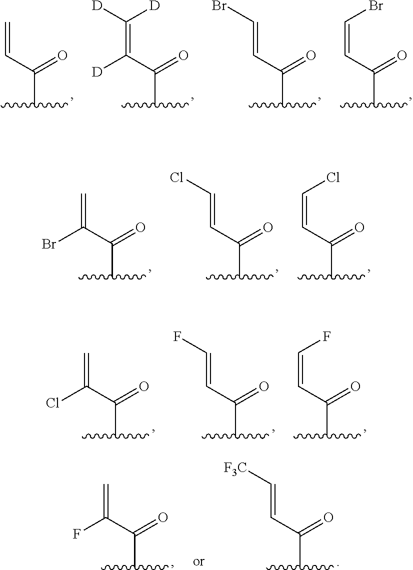

Provided herein as Embodiment 20 is the compound or salt of Embodiment 19, wherein

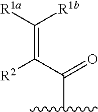

is

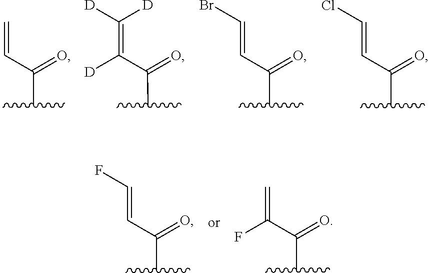

Provided herein as Embodiment 21 is the compound or salt of any one of Embodiments 1-20, wherein m is 0.

Provided herein as Embodiment 22 is the compound or salt of any one of Embodiments 1-20, wherein m is 1.

Provided herein as Embodiment 23 is the compound or salt of any one of Embodiments 1-20, wherein m is 2.

Provided herein as Embodiment 24 is the compound or salt of any one of Embodiments 1-20, wherein m is 3.

Provided herein as Embodiment 25 is the compound or salt of any one of Embodiments 1-20, wherein m is 4.

Provided herein as Embodiment 26 is the compound of salt of any one of Embodiments 22-25, wherein at least one R³ is C₁₋₃alkyl or C₁₋₃haloalkyl.

Provided herein as Embodiment 27 is the compound or salt of Embodiment 26, wherein at least one R³ is CH₃, CH₂CH₃, CF₃, CHF₂, or CH₂F.

Provided herein as Embodiment 28 is the compound or salt of any one of Embodiments 22-25, wherein at least one R³ is C₀₋₃alkyleneCN.

Provided herein as Embodiment 29 is the compound or salt of Embodiment 28, wherein at least one R³ is CN or CH₂CN.

Provided herein as Embodiment 30 is the compound or salt of any one of Embodiments 22-25, wherein at least one R³ is C₀₋₆alkyleneOH or C₀₋₃alkylene-C₁₋₃alkoxy.

Provided herein as Embodiment 31 is the compound or salt of Embodiment 30, wherein at least one R³ is OH, CH₂OH, CH₂CH₂OH, OCH₃, CH₂OCH₃, or CH₂CH₂OCH₃.

Provided herein as Embodiment 32 is the compound or salt of any one of Embodiments 22-25, wherein at least one R³ is oxo.

Provided herein as Embodiment 33 is the compound or salt of any one of Embodiments 22-25, wherein at least one R³ is spiro-cycloalkyl having 3-7 total ring atoms or spiro-heterocycloalkyl having 3-7 total ring atoms and 1 or 2 heteroatoms selected from N, O and S.

Provided herein as Embodiment 34 is the compound or salt of Embodiment 33, wherein at least one R³ is spiro-cyclopropyl, spiro-cyclobutyl, spiro-oxetanyl, or spiro-tetrahydrofuranyl.

Provided herein as Embodiment 35 is the compound or salt of any one of Embodiments 22-25, wherein two adjacent R³, together with the atoms to which they are attached, form a fused cycloalkyl ring having 3-7 total ring atoms.

Provided herein as Embodiment 36 is the compound or salt of Embodiment 35, wherein two adjacent R³, together with the atoms to which they are attached, form a fused cyclopropyl ring or a fused cyclobutyl ring.

Provided herein as Embodiment 37 is the compound or salt of any one of Embodiments 22-25, wherein each R³ independently is CH₃, CH₂CH₃, CF₃, CHF₂, CH₂F. CN, CH₂CN, OH, CH₂OH. CH₂CH₂OH, OCH₃, CH₂OCH₃, CH₂CH₂OCH₃, oxo, spiro-cyclopropyl, spiro-cyclobutyl, spiro-oxetanyl, or spiro-tetrahydrofuranyl.

Provided herein as Embodiment 38 is the compound or salt of any one of Embodiments 1-20, wherein m is 0; or m is 1 and R³ is CH₃, CF₃, CHF₂, CH₂F, CN, CH₂CN, CH₂OH, CH₂OCH₃, or spiro-oxetanyl.

Provided herein as Embodiment 39 is the compound or salt of any one of Embodiments 1-20, wherein is

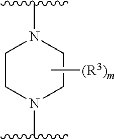

is

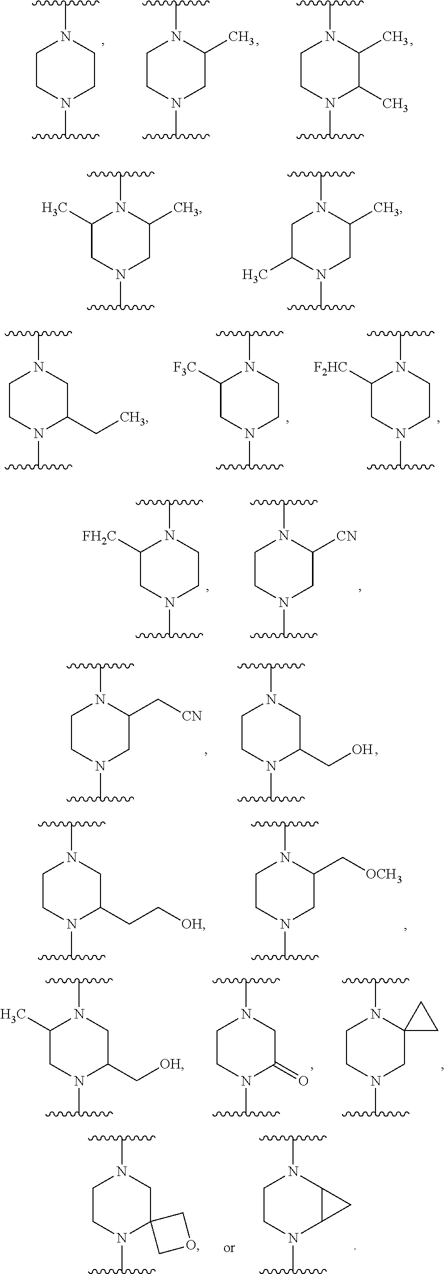

Provided herein as Embodiment 40 is the compound or salt of Embodiment 39, wherein

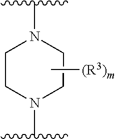

is

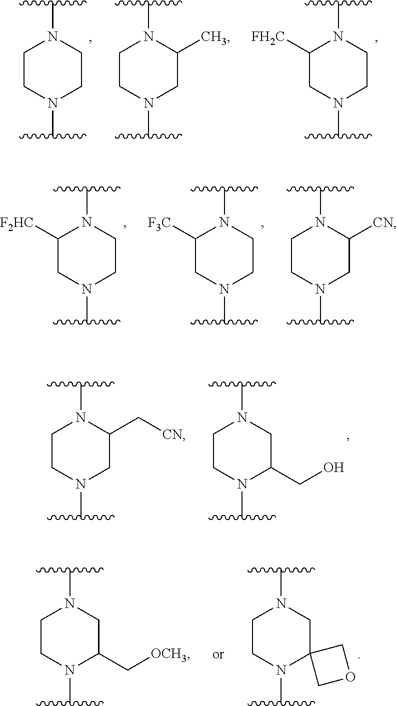

Provided herein as Embodiment 41 is the compound or salt of any one of Embodiments 1-40, wherein A is N.

Provided herein as Embodiment 42 is the compound or salt of any one of Embodiments 1-40, wherein A is CH, C-halo, C—CN, C—C₁₋₃alkyl, C—C₁₋₃haloalkyl, C—C₀₋₃alkyleneOH, or C—C₀₋₃alkylene-C₁₋₄alkoxy.

Provided herein as Embodiment 43 is the compound or salt of Embodiment 42, wherein A is CH.

Provided herein as Embodiment 44 is the compound or salt of Embodiment 42, wherein A is C—F, C—Cl, or C—CN.

Provided herein as Embodiment 45 is the compound or salt of Embodiment 42, wherein A is C—C₁₋₃alkyl or C—C₁₋₃haloalkyl.

Provided herein as Embodiment 46 is the compound or salt of Embodiment 45, wherein A is C—CH₃, C—CH₂F, C—CHF₂, or C—CF₃.

Provided herein as Embodiment 47 is the compound or salt of Embodiment 42, wherein A is C—C₀₋₃alkyleneOH or C—C₀₋₃alkylene-C₁₋₄alkoxy.

Provided herein as Embodiment 48 is the compound or salt of Embodiment 47, wherein A is C—OH, C—CH₂OH, C—OCH₃, or C—CH₂OCH₃.

Provided herein as Embodiment 49 is the compound or salt of any one of Embodiments 1-48, wherein n is 0.

Provided herein as Embodiment 50 is the compound or salt of any one of Embodiments 1-48, wherein n is 1.

Provided herein as Embodiment 51 is the compound or salt of any one of Embodiments 1-48, wherein n is 2.

Provided herein as Embodiment 52 is the compound or salt of Embodiment 50 or 51, wherein at least one R⁴ is C₁₋₆alkyl or C₁₋₃haloalkyl.

Provided herein as Embodiment 53 is the compound or salt of Embodiment 52, wherein at least one R⁵ is CH₃, CH₂CH₃, CH₂CH₂CH₃, CH(CH₃)₂, CF₃, CHF₂, or CH₂F.

Provided herein as Embodiment 54 is the compound or salt of Embodiment 50 or 51, wherein at least one R⁴ is C₀₋₂alkyleneCN.

Provided herein as Embodiment 55 is the compound or salt of Embodiment 54, wherein at least one R⁴ is CN or CH₂CN.

Provided herein as Embodiment 56 is the compound or salt of Embodiment 50 or 51, wherein at least one R⁴ is C₁₋₃alkyleneOH or C₁₋₃alkylene-C₁₋₃alkoxy.

Provided herein as Embodiment 57 is the compound or salt of Embodiment 56, wherein at least one R⁴ is CH₂OH, CH₂CH₂OH, CH₂OCH₃, or CH₂CH₂OCH₃.

Provided herein as Embodiment 58 is the compound or salt Embodiment 50 or 51, wherein at least one R⁴ is oxo.

Provided herein as Embodiment 59 is the compound or salt of Embodiment 50 or 51, wherein at least one R⁴ is spiro-cycloalkyl having 3-7 total ring atoms or spiro-heterocycloalkyl having 4-7 total ring atoms and 1 or 2 heteroatoms selected from N, O and S.

Provided herein as Embodiment 60 is the compound or salt of Embodiment 59, wherein at least one R⁴ is spiro-cyclopropyl, spiro-cyclobutyl, or spiro-oxetanyl.

Provided herein as Embodiment 61 is the compound or salt of Embodiment 41, wherein

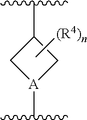

is

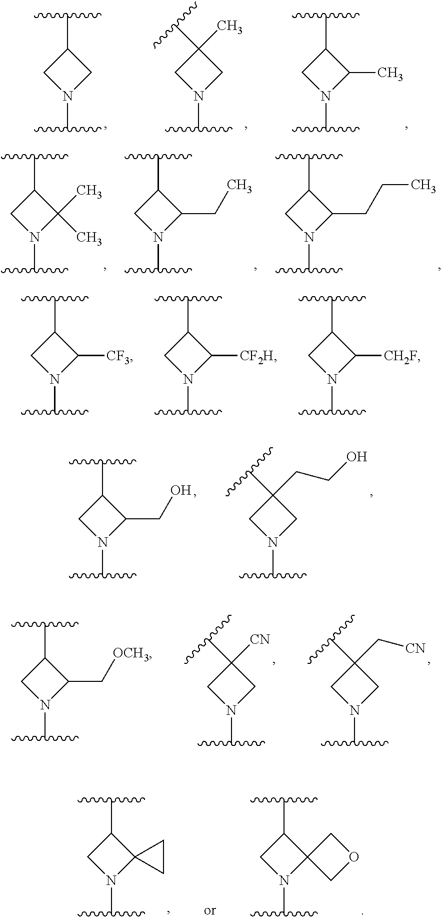

Provided herein as Embodiment 62 is the compound or salt of Embodiment 61, wherein

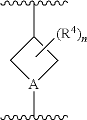

is

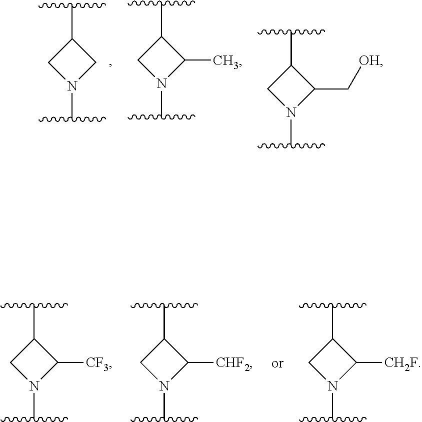

Provided herein as Embodiment 63 is the compound or salt of Embodiment 62, wherein

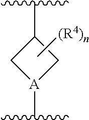

is

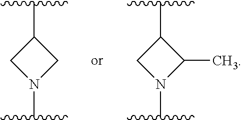 [SMILES: *C1CN(*)C1 ; *C1CN(*)C1C]

Provided herein as Embodiment 64 is the compound or salt of Embodiment 42, wherein

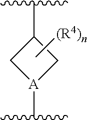

is

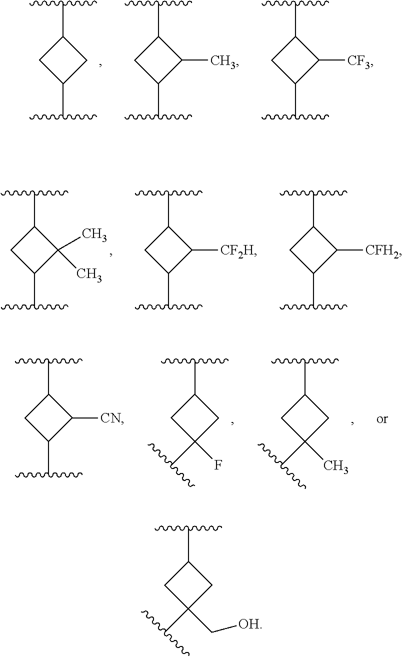

Provided herein as Embodiment 65 is the compound or salt of any one of Embodiments 1-64, wherein W is CH.

Provided herein as Embodiment 66 is the compound or salt of any one of Embodiments 1-64, wherein W is C—F, C—Cl, or C—CN.

Provided herein as Embodiment 67 is the compound or salt of any one of Embodiments 1-64, wherein W is C—C₁₋₃alkyl or C—C₁₋₃haloalkyl.

Provided herein as Embodiment 68 is the compound or salt of Embodiment 67, wherein W is C—CH₃ or C—CH₂CH₃.

Provided herein as Embodiment 69 is the compound or salt of any one of Embodiments 1-64, wherein W is C—C₀₋₂alkyleneOH or C—C₀₋₃alkylene-C₁₋₄alkoxy.

Provided herein as Embodiment 70 is the compound or salt of Embodiment 69, wherein W is C—OH, C—CH₂OH, C—OCH₃, or C—CH₂OCH₃.

Provided herein as Embodiment 71 is the compound or salt of any one of Embodiments 1-70, wherein R^{5a} is H.

Provided herein as Embodiment 72 is the compound or salt of any one of Embodiments 1-70, wherein R^{5a} is CN.

Provided herein as Embodiment 73 is the compound or salt of any one of Embodiments 1-70, wherein R^{5a} is Br, Cl, or F.

Provided herein as Embodiment 74 is the compound or salt of any one of Embodiments 1-70, wherein R^{5a} is C₁₋₃alkyl or C₁₋₃haloalkyl.

Provided herein as Embodiment 75 is the compound or salt of Embodiment 74, wherein R^{5a} is CH₃, CH₂CH₃, CF₃, CHF₂, or CH₂F.

Provided herein as Embodiment 76 is the compound or salt of Embodiment 75, wherein R^{1a} is CH₃.

Provided herein as Embodiment 77 is the compound or salt of any one of Embodiments 1-70, wherein R^{5a} is C₂₋₆alkenyl or C₂₋₃alkynyl.

Provided herein as Embodiment 78 is the compound or salt of Embodiment 77, wherein R^{5a} is

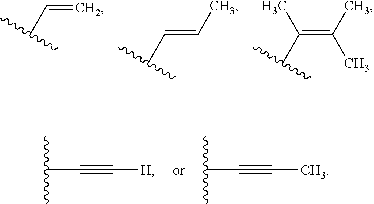

Provided herein as Embodiment 79 is the compound or salt of Embodiment 78, wherein R⁵ª is

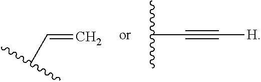

Provided herein as Embodiment 80 is the compound or salt of any one of Embodiments 1-70, wherein R^{5a} is C₀₋₆alkyleneOH, C₀₋₃alkylene-C₁₋₃alkoxy, or cycloalkyl having 3-5 total ring atoms.

Provided herein as Embodiment 81 is the compound or salt of Embodiment 80, wherein R^{5s} is OH, CH₂OH, OCH₃, CH₂OCH₃, or

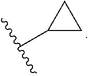 [SMILES: *C1CC1]

Provided herein as Embodiment 82 is the compound or salt of any one of Embodiments 1-70, wherein R^{5a} and an R⁴, together with the atoms to which they are attached, form an optionally substituted ring having 6-10 total ring atoms and 0, 1, or 2 heteroatoms selected from N, O, and S, wherein the ring is saturated or unsaturated.

Provided herein as Embodiment 83 is the compound or salt of Embodiment 82, wherein the optionally substituted ring is saturated.

Provided herein as Embodiment 84 is the compound or salt of Embodiment 82, wherein the optionally substituted ring is unsaturated.

Provided herein as Embodiment 85 is the compound or salt of any one of Embodiments 82-84, wherein the optionally substituted ring has 6 total ring atoms.

Provided herein as Embodiment 86 is the compound or salt of any one of Embodiments 82-85, wherein the optionally substituted ring has 7 total ring atoms.

Provided herein as Embodiment 87 is the compound or salt of any one of Embodiments Embodiment 82-85, wherein the optionally substituted ring has 8 total ring atoms.

Provided herein as Embodiment 88 is the compound or salt of any one of Embodiments 82-85, wherein the optionally substituted ring has 9 or 10 total ring atoms.

Provided herein as Embodiment 89 is the compound or salt of any one of Embodiments 82-88, wherein the optionally substituted ring has 0 heteroatoms.

Provided herein as Embodiment 90 is the compound or salt of any one of Embodiments 82-88, wherein the optionally substituted ring bas 1 or 2 heteroatoms selected from N, O, and S.

Provided herein as Embodiment 91 is the compound or salt of Embodiment 90, wherein the 1 or 2 heteroatoms are each O.

Provided herein as Embodiment 92 is the compound or salt of Embodiment 91, wherein the optionally substituted ring is an ether.

Provided herein as Embodiment 93 is the compound or salt of Embodiment 91, wherein the 1 or 2 heteroatoms are each N.

Provided herein as Embodiment 94 is the compound or salt of Embodiment 93, wherein the ring is a lactam or a cyclic amine

Provided herein as Embodiment 95 is the compound or salt of any one of Embodiments 82-94, wherein the ring is unsubstituted.

Provided herein as Embodiment 96 is the compound or salt of any one of Embodiments 82-94, wherein the ring is substituted with 1 or 2 substituents selected from the group consisting of C₁₋₃alkyl, C₁₋₃haloalkyl, oxo, halo, CN, C₀₋₆alkyleneOH, C₀₋₃alkylene-C₁₋₃alkoxy, cycloalkyl having 3-7 total ring atoms, cycloalkenyl having 5-7 total ring atoms, heterocycloalkyl having 4-7 total ring atoms and 1-3 heteroatoms selected from N, O, and S, heterocycloalkenyl having 5-7 total ring atoms and 1-3 heteroatoms selected from N, O, and S, and phenyl.

Provided herein as Embodiment 97 is the compound or salt of Embodiment 82, wherein

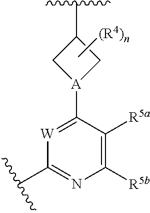

is

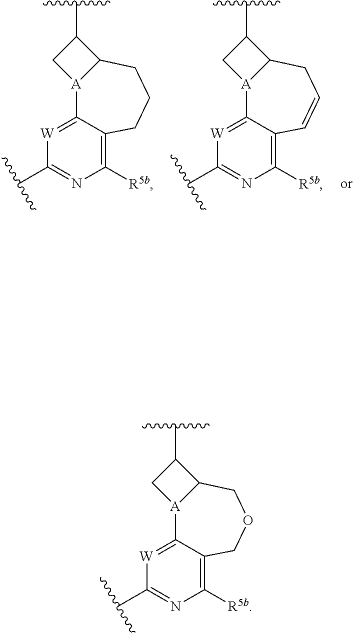

Provided herein as Embodiment 98 is the compound or salt of any one of Embodiments 1-97, wherein R^{5b} is C₁₋₄haloalkyl.

Provided herein as Embodiment 99 is the compound or salt of Embodiment 98, wherein R^{5b} is CF₃, CF₂H, CFH₂, or CF₂CH₃.

Provided herein as Embodiment 100 is the compound or salt of any one of Embodiments 1-97, wherein R^{5b} is Br, Cl, or F.

Provided herein as Embodiment 101 is the compound or salt of any one of Embodiments 1-97, wherein R^{5b} is C₁₋₃alkoxy or C₁₋₃thioalkoxy.

Provided herein as Embodiment 102 is the compound or salt of Embodiment 101, wherein R^{5b} is OCH₃, or SCH₃.

Provided herein as Embodiment 103 is the compound or salt of any one of Embodiments I-97, wherein R⁵ is C₁₋₄alkyl, C₂₋₃alkenyl, or C₂₋₃alkynyl, optionally wherein each of the alkyl, alkenyl, and alkynyl is independently substituted with 1, 2, or 3 substituents selected from C₁₋₃alkyl, C₁₋₃haloalkyl, C₀₋₃alkylene (OH), C₀₋₆alkylene-C₁₋₃alkoxy, cycloalkyl having 3-7 total ring atoms, cycloalkenyl having 5-7 total ring atoms, heterocycloalkyl having 3-7 total ring atoms and 1-3 heteroatoms selected from N, O, and S, heterocycloalkenyl having 3-7 total ring atoms and 1-3 heteroatoms selected from N, O, and S, and phenyl.

Provided herein as Embodiment 104 is the compound or salt of Embodiment 103, wherein each of the 1, 2, or 3 substituents independently is selected from CH₃, CF₃, CF₂H, CFH₂, OH, OCH₃, OCF₃, CH₂OH, CH₂OCH₃, cyclopropyl, cyclobutyl, and phenyl.

Provided herein as Embodiment 105 is the compound or salt of Embodiment 103, wherein R^{5b} is CH₃, CH₂CH₃, CH₂CH₂CH₃, CH(CH₃)₂,

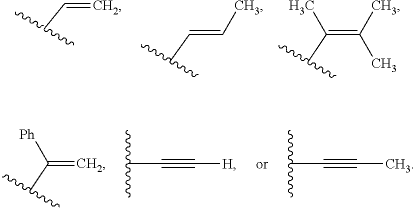

Provided herein as Embodiment 106 is the compound or salt of any one of Embodiments 1-97, wherein R^{5a} and R^{5b}, together with the atoms to which they are attached, form a fused cycloalkyl ring having 3-7 total ring atoms.

Provided herein as Embodiment 107 is the compound or salt of Embodiment 106, wherein R^{5a} and R^{5b}, together with the atoms to which they are attached, form fused-cyclopropyl, fused-cyclobutyl, or fused-cyclopentyl.

Provided herein as Embodiment 108 is the compound or salt of any one of Embodiments 1-64, wherein W is CH, R^{5a} is CN, Br, Cl, F, or CH₃, and R^{5b} is CF₃, CF₂H, or CFH₂.

Provided herein as Embodiment 109 is the compound or salt of any one of Embodiments 1-64, wherein

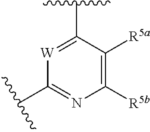

is

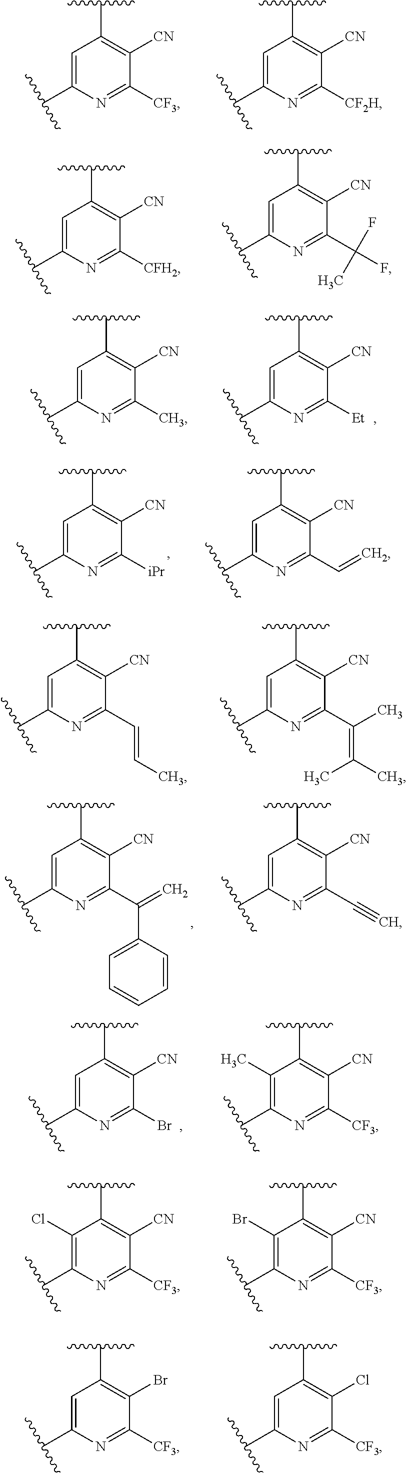

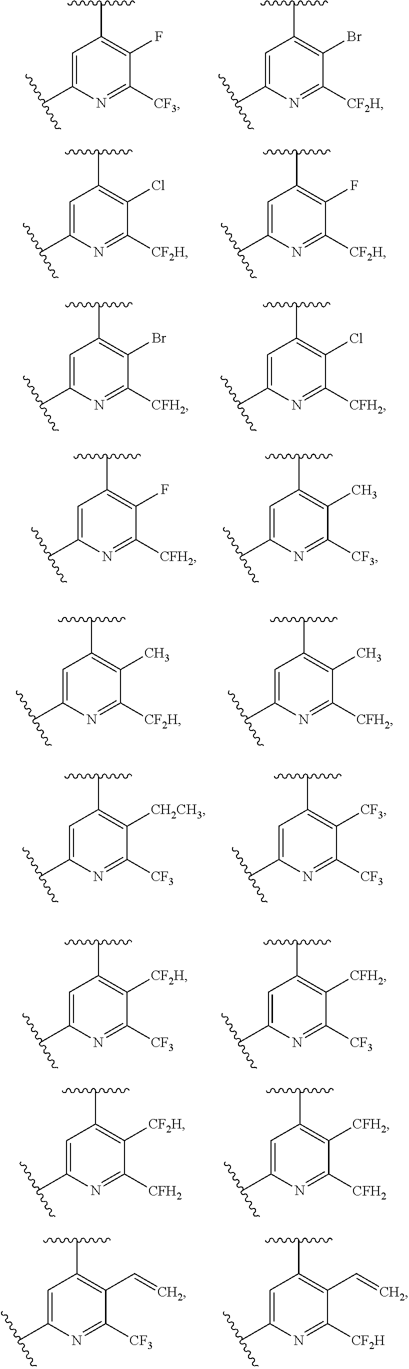

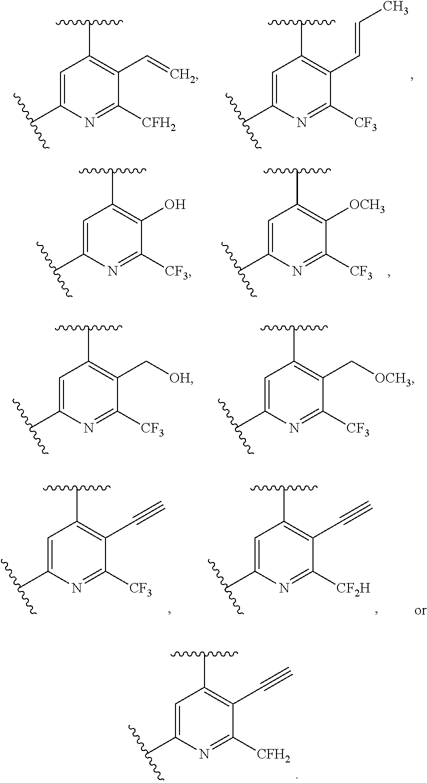

Provided herein as Embodiment 110 is the compound or salt of Embodiment 109, wherein

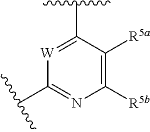

is

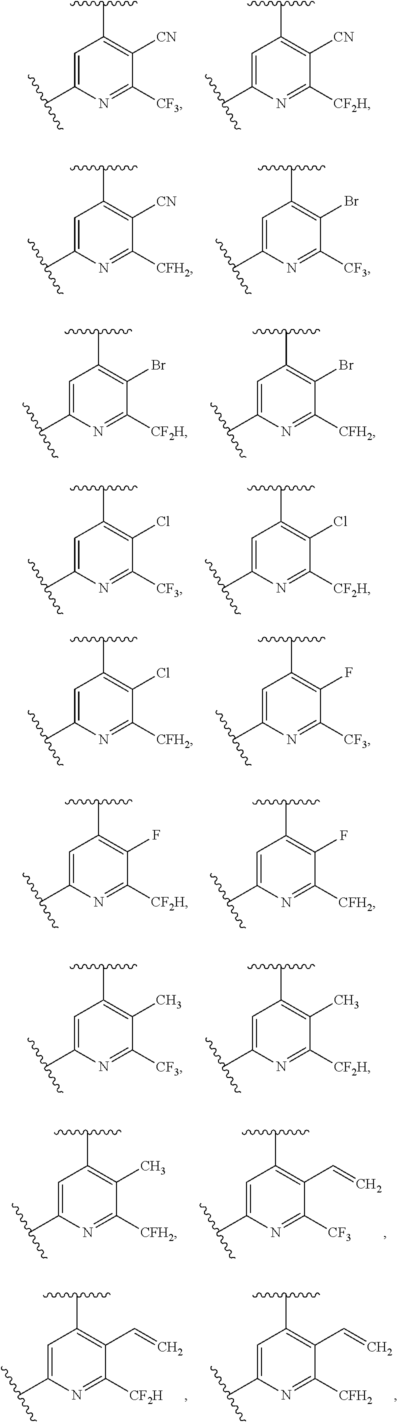

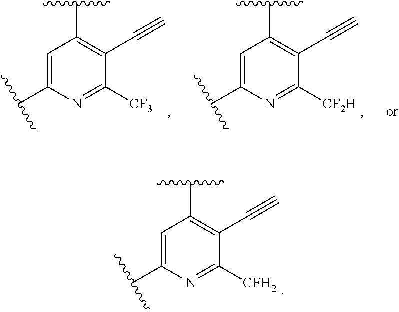

Provided herein as Embodiment 111 is the compound or salt of Embodiment 110, wherein

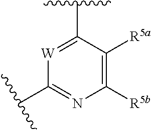

is

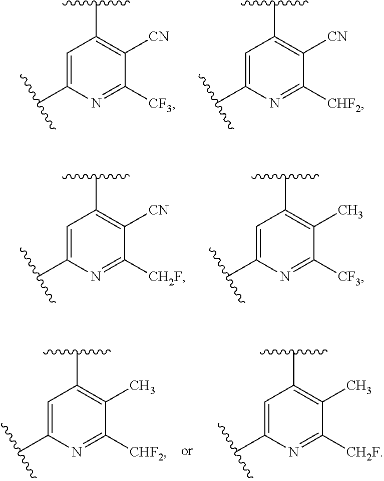

Provided herein as Embodiment 112 is the compound or salt of any one of Embodiments 1-111, wherein X is

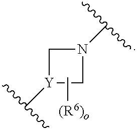

Provided herein as Embodiment 113 is the compound or salt of any one of Embodiments 1-111, wherein X is

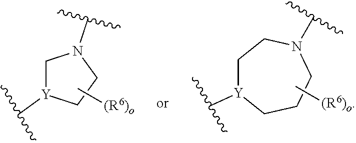

Provided herein as Embodiment 114 is the compound or salt of any one of Embodiments 1-111, wherein X is

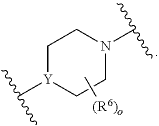

Provided herein as Embodiment 115 is the compound or salt of any one of Embodiments 1-111, wherein X is

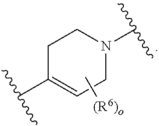

Provided herein as Embodiment 116 is the compound or salt of any one of Embodiments 1-114, wherein Y is N.

Provided herein as Embodiment 117 is the compound or salt of any one of Embodiments 1-114, wherein Y is C—H.

Provided herein as Embodiment 118 is the compound or salt of any one of Embodiments 1-114, wherein Y is C-halo, C—CN, C—C₁₋₃alkyl, C—C₁₋₃haloalkyl, C—C₀₋₂alkyleneOH, or C—C₀₋₃alkylene-C₁₋₄alkoxy.

Provided herein as Embodiment 119 is the compound or salt of Embodiment 118, wherein Y is C—F, C—Cl, C₂CH₃, C—CH₂CH₃, C—CH₂F, C—CHF₂, C—CF₃, C—OH, C—CH₂OH, C—OCH₃, or C—CH₂OCH₃.

Provided herein as Embodiment 120 is the compound or salt of any one of Embodiments 1-119, wherein o is 0.

Provided herein as Embodiment 121 is the compound or salt of any one of Embodiments]-119, wherein o is 1.

Provided herein as Embodiment 122 is the compound or salt of any one of Embodiments 1-119, wherein o is 2.

Provided herein as Embodiment 123 is the compound or salt of any one of Embodiments 1-119, wherein o is 3.

Provided herein as Embodiment 124 is the compound or salt of any one of Embodiments 1-119, wherein o is 4.

Provided herein as Embodiment 125 is the compound or salt of any one of Embodiments 121-124, wherein at least one R⁶ is Br, Cl, F. CN, or oxo.

Provided herein as Embodiment 126 is the compound or salt of Embodiment 125, wherein at least one R⁵ is F.

Provided herein as Embodiment 127 is the compound or salt of any one of Embodiments 121-124, wherein at least one R⁶ is C₁₋₃alkyl or C₁₋₃haloalkyl.

Provided herein as Embodiment 128 is the compound or salt of Embodiment 127, wherein at least one R⁶ is CH₃, CH₂F, CHF₂, or CF₃.

Provided herein as Embodiment 129 is the compound or salt of any one of Embodiments 121-124, wherein at least one R⁶ is C₀₋₆alkyleneOH, C₀₋₃alkylene-C₁₋₄alkoxy, deuterated C₀₋₆alkylene-C₁₋₃alkoxy, or C₁₋₂alkylene-N(R^{N1})₂, and each R^{N1} independently is H or CH₃.

Provided herein as Embodiment 130 is the compound or salt of Embodiment 129, wherein at least one R⁶ is OH, CH₂OH, OCH₃, OCD₃, or CH₂OCH₃.

Provided herein as Embodiment 131 is the compound or salt of any one of Embodiments 121-124, wherein at least one R⁶ is spiro-cycloalkyl having 3-7 total ring atoms or spiro-heterocycloalkyl having 4-7 total ring atoms and 1 or 2 heteroatoms selected from N, O and S.

Provided herein as Embodiment 132 is the compound or salt of Embodiment 131, wherein at least one R⁶ is spiro-cyclopropyl, spiro-cyclobutyl, spiro-oxetanyl, or spiro-tetrahydrofuranyl.

Provided herein as Embodiment 133 is the compound or salt of Embodiment 132, wherein R⁶ is spiro-cyclopropyl.

Provided herein as Embodiment 134 is the compound or salt of any one of Embodiments 121-124, wherein two adjacent R⁶, together with the atoms to which they are attached, form a fused cycloalkyl ring having 3-7 total ring atoms; or Y and an adjacent R⁶, together with the atoms to which they are attached, form a fused cycloalkyl ring having 3-7 total ring atoms; wherein the fused cycloalkyl ring of any of the foregoing is optionally substituted with 1 or 2 substituents selected from halo, OH, C₁₋₃alkoxy, or CN.

Provided herein as Embodiment 135 is the compound or salt of Embodiment 134, wherein the fused cycloalkyl ring is a fused-cyclopropyl, fused-cyclobutyl, or fused-cyclopentyl ring.

Provided herein as Embodiment 136 is the compound or salt of any one of Embodiments 121-124, wherein two non-adjacent R⁶ join together to form a C₁₋₃alkylene bridge or a C₁₋₃ether bridge.

Provided herein as Embodiment 137 is the compound of salt of Embodiment 136, wherein

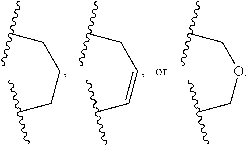

two non-adjacent R⁶ join together to form

Provided herein as Embodiment 138 is the compound of salt of any one of Embodiments 1-112, wherein X is

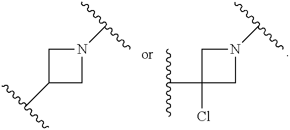 [SMILES: *C1CN(*)C1 ; *N1CC(*)(Cl)C1]

Provided herein as Embodiment 139 is the compound or salt of any one of Embodiments 1-111 and 113, wherein X

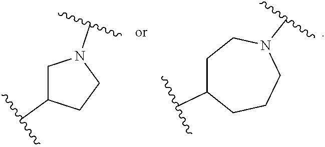 [SMILES: *C1CCN(*)C1 ; *C1CCCN(*)CC1]

Provided herein as Embodiment 140 is the compound or salt of any one of Embodiments 1-111 and 114, wherein X is

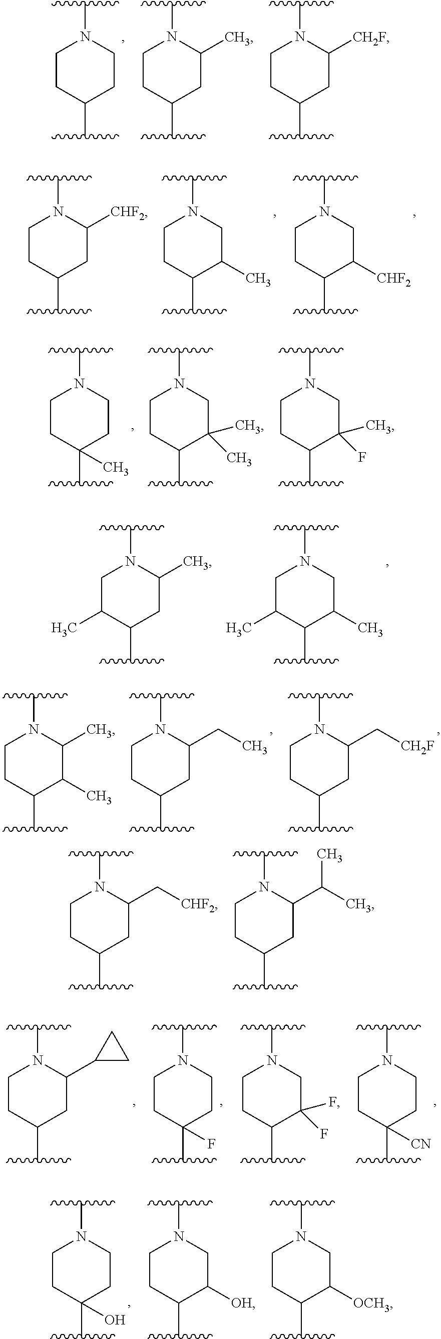

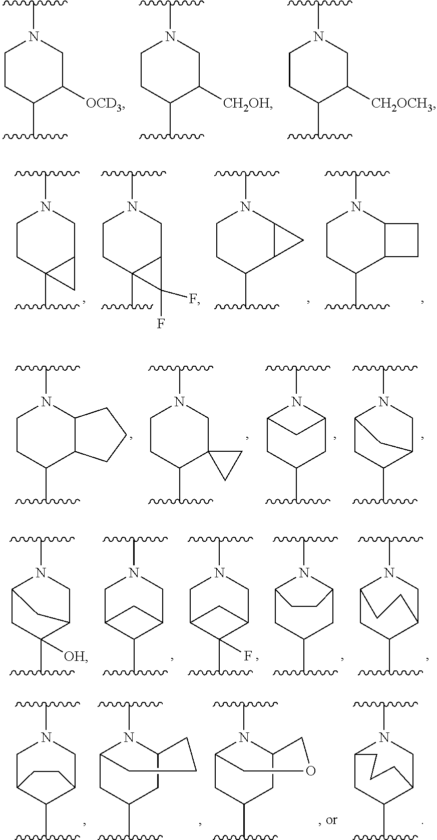

Provided herein as Embodiment 141 is the compound or salt of any one of Embodiments 1-111 and 114, wherein X is

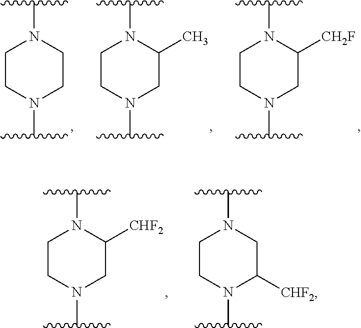

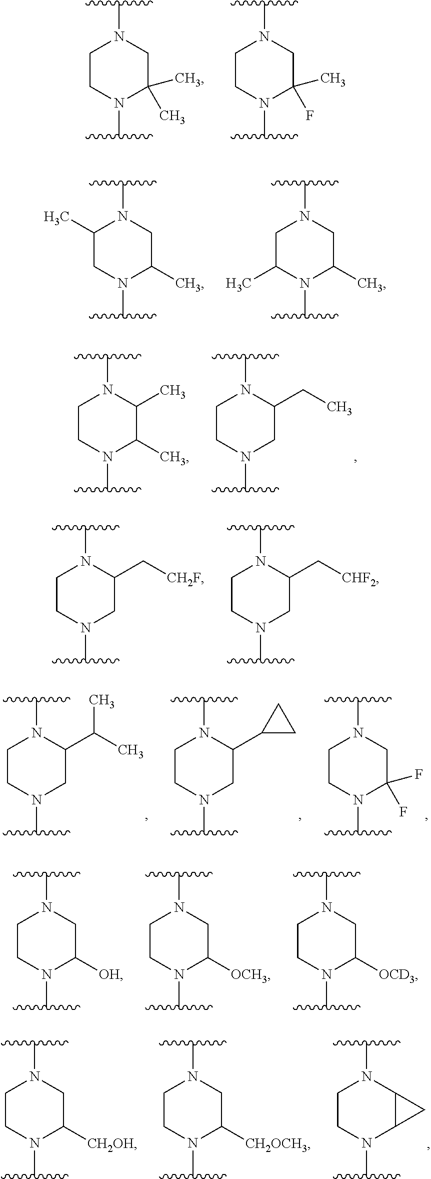

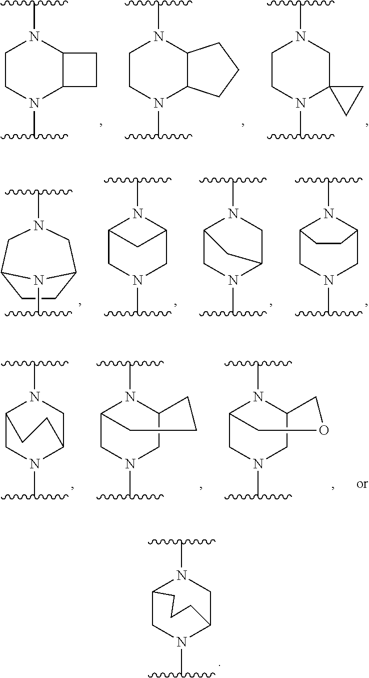

Provided herein as Embodiment 142 is the compound or salt of any one of Embodiments 1-111 and 115, wherein X is

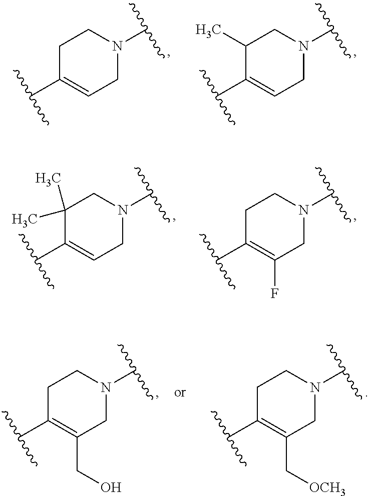

Provided herein as Embodiment 143 is the compound or salt of any one of Embodiments 1-142, wherein Z is phenyl optionally substituted with 1-4 substituents selected from halo, C₀₋₃alkyleneCN, C₀₋₃alkyleneOH, C₀₋₃alkylene-C₁₋₃alkoxy, C₀₋₂alkylene-C₁₄thioalkoxy, and

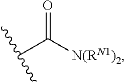

wherein each RN′ independently H or CH₃.

Provided herein as Embodiment 144 is the compound or salt of Embodiment 143, wherein each of the 1-4 substituents independently is selected from F, Cl, CN, OCH₃, SCH₃, CH₂OH, and

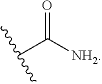 [SMILES: *C(N)=O]

Provided herein as Embodiment 145 is the compound or salt of Embodiment 143, wherein Z is

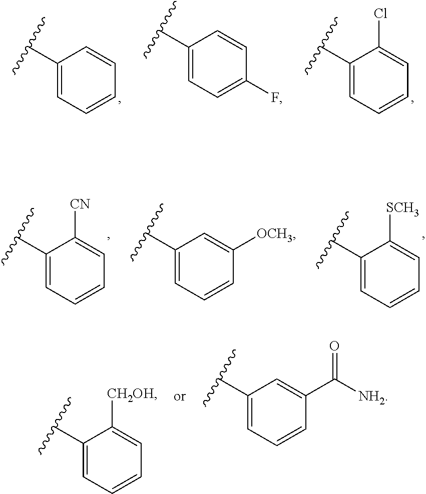

Provided herein as Embodiment 146 is the compound or salt of any one of Embodiments I-142, wherein Z is heteroaryl comprising 5 or 6 total ring atoms and 1-3 heteroatoms selected from N, O, and S, wherein the heteroaryl is optionally substituted with 1-4 substituents.

Provided herein as Embodiment 147 is the compound or salt of Embodiment 146, wherein the heteroaryl is pyrrolyl, furanyl, thiophenyl, pyrazolyl, imidazolyl, thiazolyl, isothiazolyl, oxazolyl, isoxazolyl, triazolyl, thiadiazolyl, oxadiazolyl, pyridyl, pyridazinyl, pyrimidinyl, pyrazinyl, or triazinyl.

Provided herein as Embodiment 148 is the compound or salt of Embodiment 147, wherein the heteroaryl is imidazolyl, thiazolyl, isothiazolyl, oxazolyl, isoxazolyl, or triazolyl.

Provided herein as Embodiment 149 is the compound or salt of Embodiment 147, wherein the heteroaryl is pyridyl, pyridazinyl, pyrimidinyl, or pyrazinyl.

Provided herein as Embodiment 150 is the compound or salt of any one of Embodiments 146-149, wherein the heteroaryl is substituted with 1-4 substituents, each of which is selected from the group consisting of halo, CN, C₁₋₆alkyl, C₁₋₆haloalkyl, C₂₋₆alkenyl, C₂₋₆haloalkenyl, C₀₋₆alkylene-OH, C₀₋₆alkylene-C₁₋₃alkoxy, C₀₋₃alkylene-N(R^{N1}); wherein each R^{N1} independently is H or C₁₋₃alkyl, C₀₋₂alkylene-cycloalkyl having 3-6 total ring atoms, C₀₋₂alkylene-heterocycloalkyl having 3-6 total ring atoms and 1-3 heteroatoms selected from N, O, and S, and C₀₋₂alkylene-phenyl; wherein each of the alkyl, alkenyl, C₀₋₆alkylene-C₁₋₃alkoxy, cycloalkyl, heterocycloalkyl, and phenyl substituents independently is optionally substituted with 1-3 substituents independently selected from deuterium, halo, OH, CH₃, OCH₃, and OCD₃.

Provided herein as Embodiment 151 is the compound or salt of Embodiment 150, wherein each of the 1-4 substituents independently is selected from the group consisting of Cl, F, CN, CH₃, CD₃, CH₂CH₃, CH(CH₃)₂, CF₃, CHF₂, CH₂F, CH₂CHF₂, CH₂CH₂F, CH(CH₂F) 2, CH(CH₃)CH₂F, CH(CH₃)CHF₂, C(═CH₂)CH₂F, OH, CH₂OH, CH₂CH₂OH, CH(CH,)CH₂OH, C(CH₃)₂OH, C(CH₃): CH₂OH, CH₂C(CH₃)₂OH, OCH₃, OCD₃, CH₂OCH₃, CH₂OCD₃, CH—CH₂OCH₃, CHFCH₂OCH₃, CF₂CH₂OCH₃, CH₂CH₂OCD₃, CH₂CH₂OCH₂CH₃,CH₂CH₂CH₂OCH₃, CH₂CH₂CH₂OCD₃, CH(CH₃)CH₂OCH₃, CH(CH₃)CH₂OCD₃,C(CH₃)₂CH₂OCH₃, C(CH₃)₂CH₂OCD₃, CH₂CH(CH₃)OCH₃, CH—CH(CH₃)OCD₃, CH₂C(CH₃)₂OCH; CH₂C(CH₃)₂OCD₃, NH₂, CH₂NH₂, CH₂NHCH₃, CH₂N(CH₃)₂, CH₂CH₂NH₂, CH₂CH₂NHCH₃, CH₂CH₂N(CH₃):

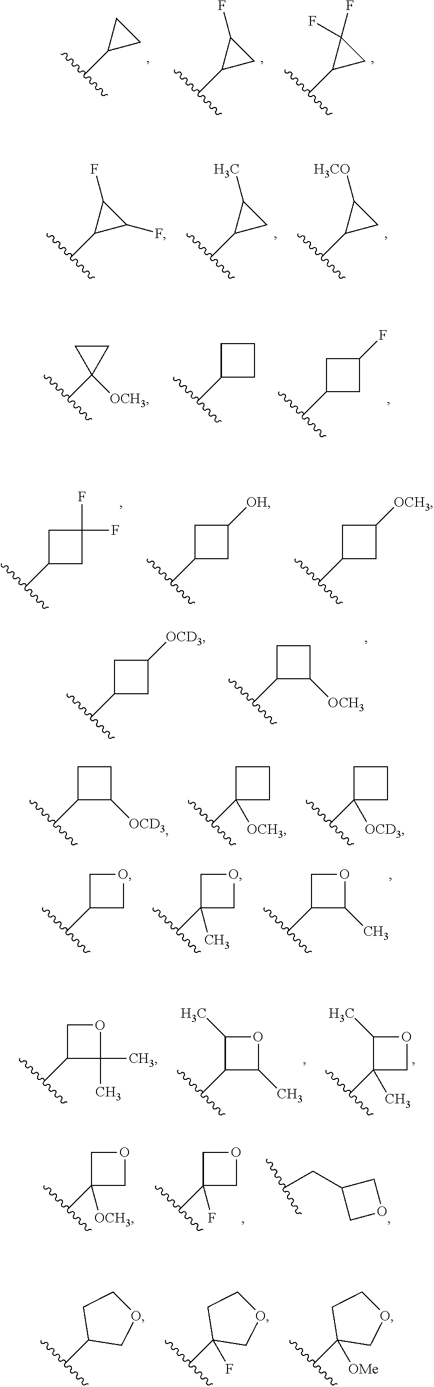

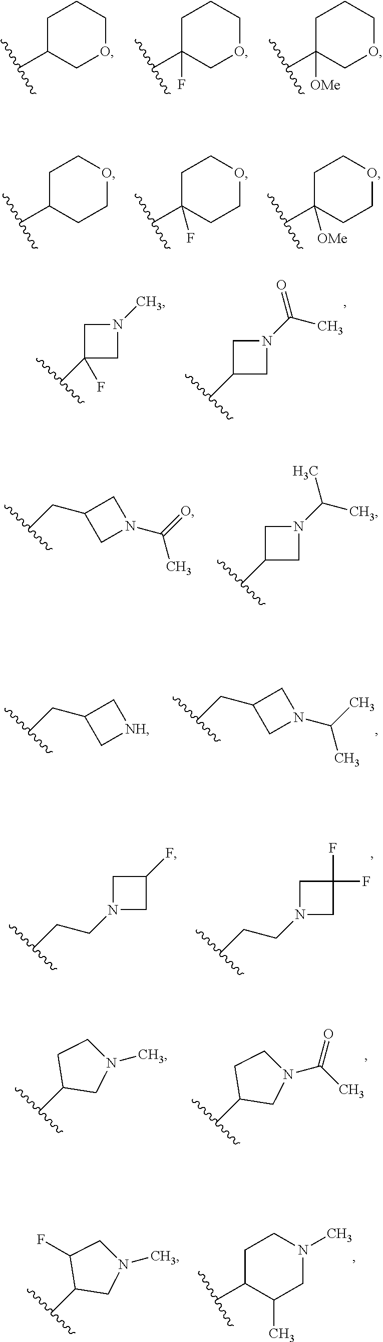

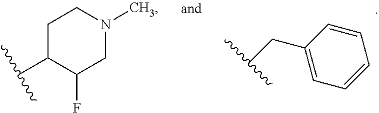 [SMILES: *C1CCN(C)CC1F ; *Cc1ccccc1]

Provided herein as Embodiment 152 is the compound or salt of Embodiment 151, wherein each of the 1-4 substituents independently is CH₃, CH(CH₃)₂. C(CH₃)₂OH, CH₂OCD₃, CH₂CH₂OCH₃, CF₂CH₂OCH₃, CH₂CH₂OCD₃, CH₂CH₂CH₂OCH₃, CH₂CH(CH)OCH₃, CH₂C(CH₃)₂OCH₃,

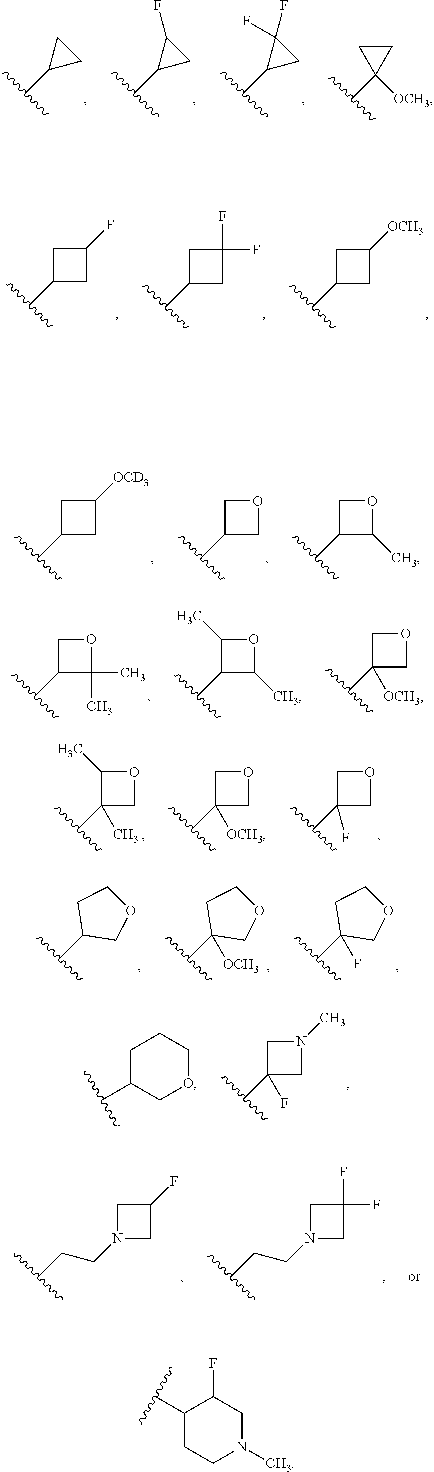

Provided herein as Embodiment 153 is the compound or salt of any one of Embodiments 146-148, wherein Z is

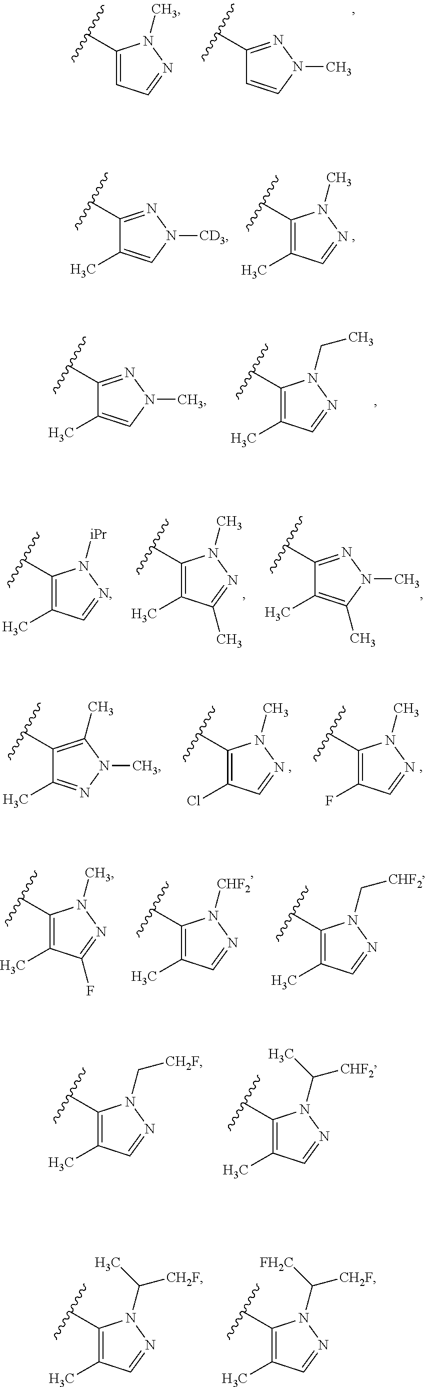

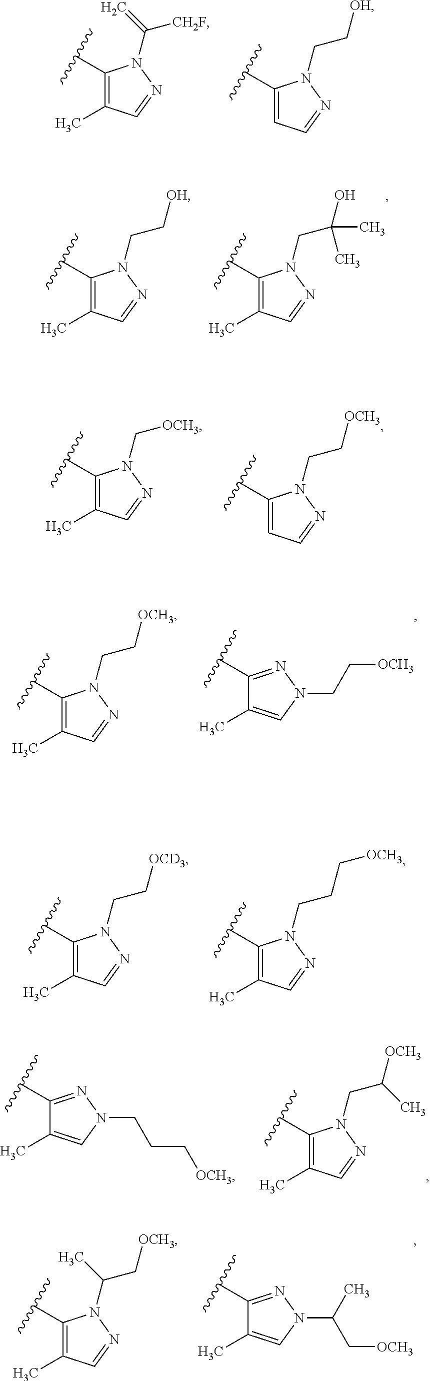

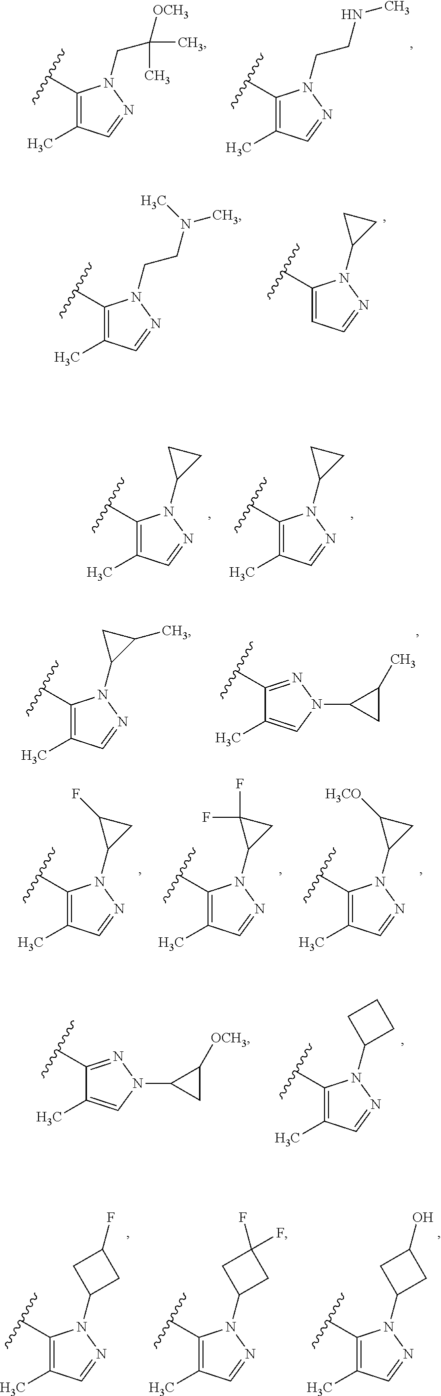

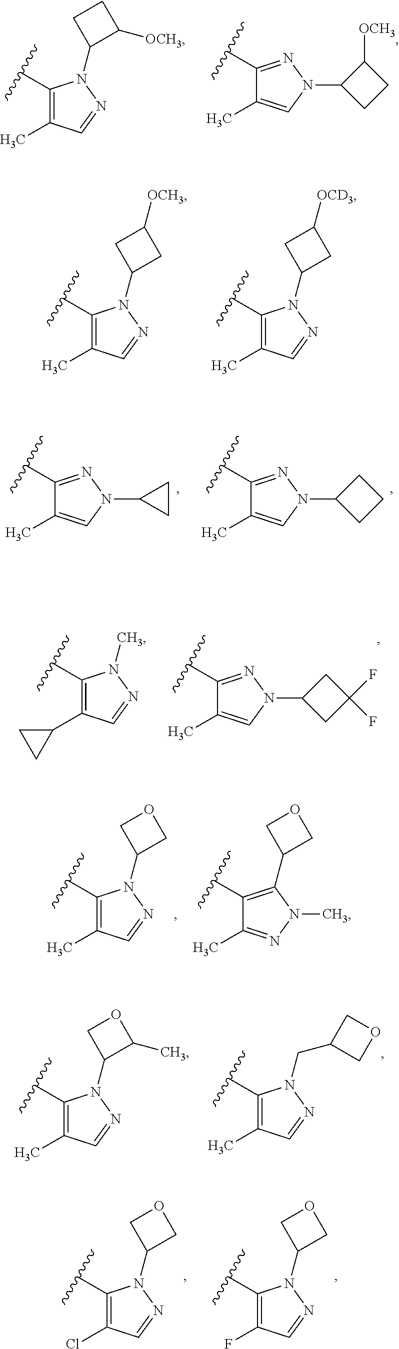

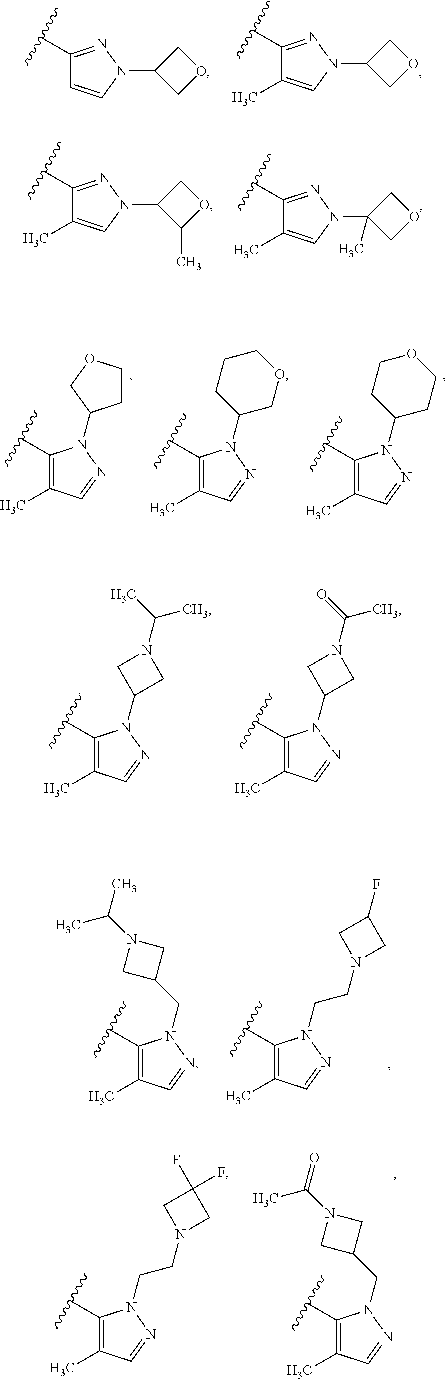

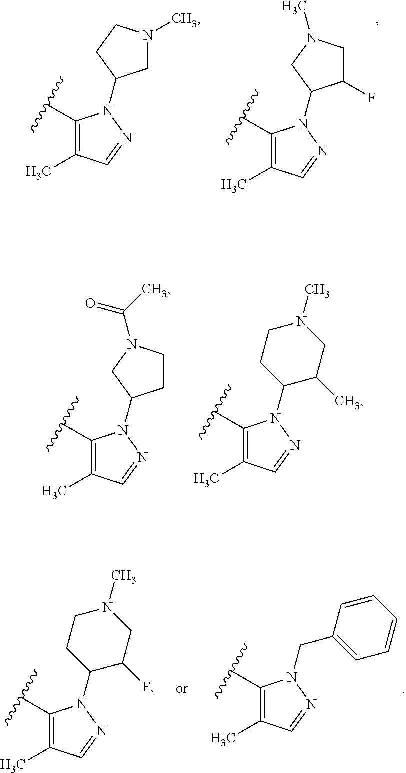 [SMILES: *c1c(C)cnn1C1CCN(C)C1 ; *c1c(C)cnn1C1CN(C)CC1F ; *c1c(C)cnn1C1CCN(C(C)=O)C1 ; *c1c(C)cnn1C1CCN(C)CC1C ; *c1c(C)cnn1C1CCN(C)CC1F ; *c1c(C)cnn1Cc1ccccc1]

Provided herein as Embodiment 154 is the compound or salt of Embodiment 153, wherein Z is

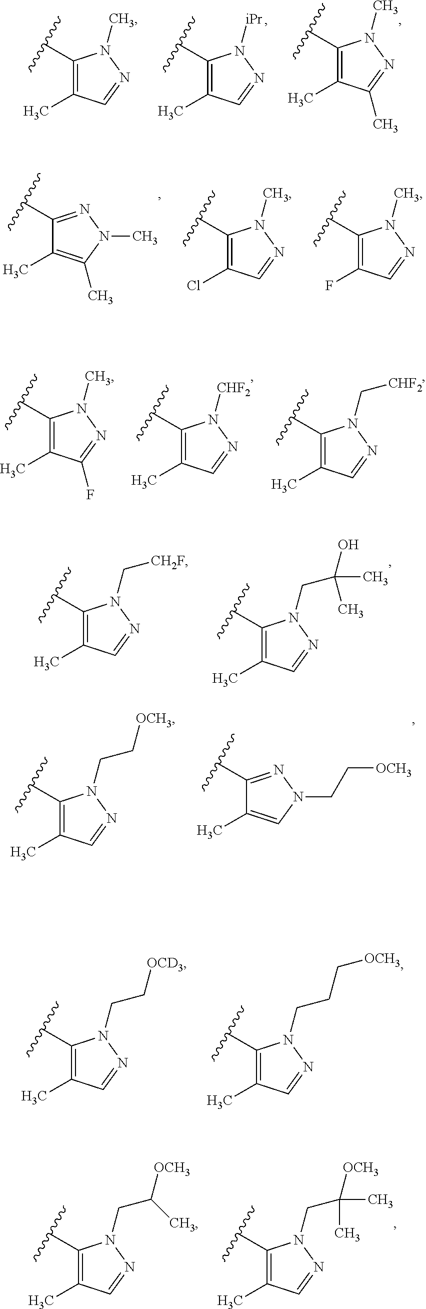

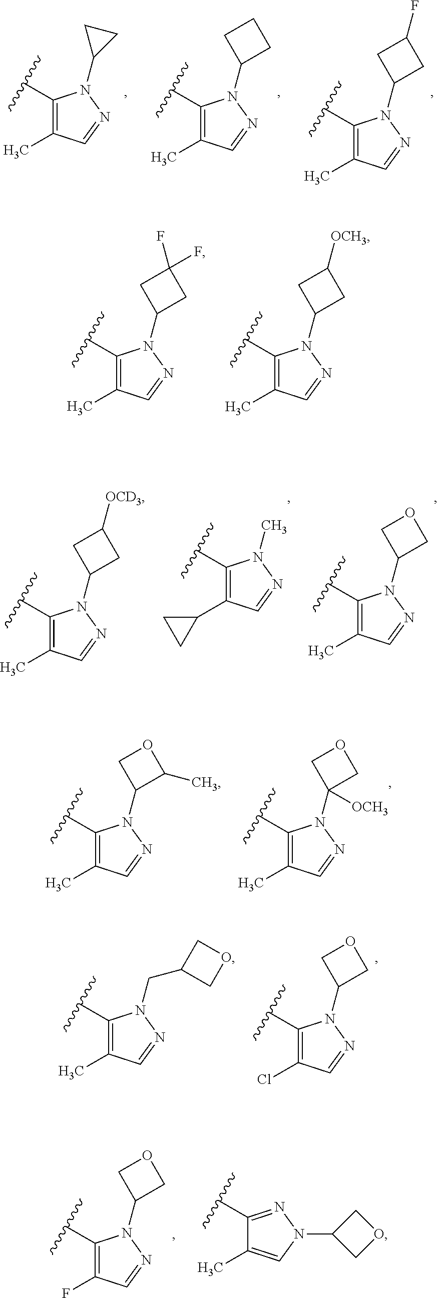

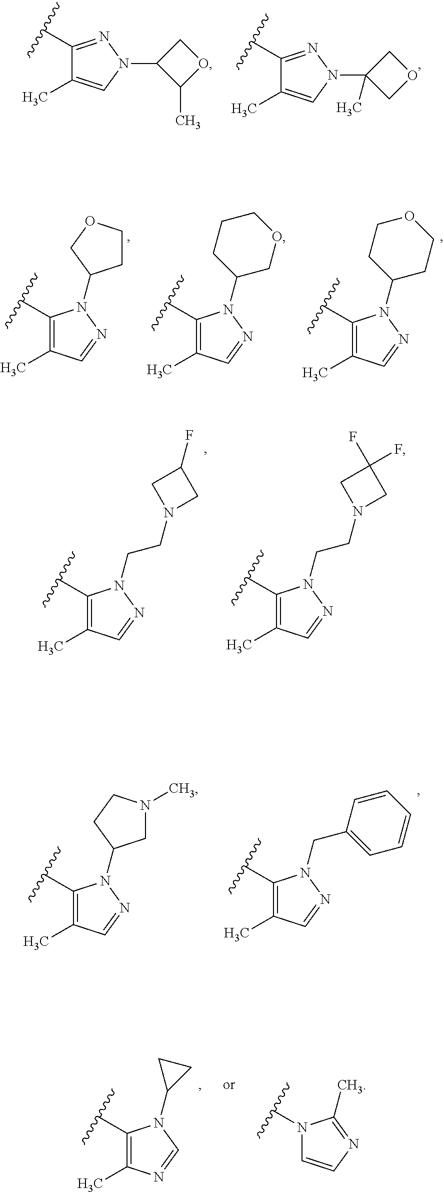

Provided herein as Embodiment 155 is the compound or salt of Embodiment 154, wherein Z is

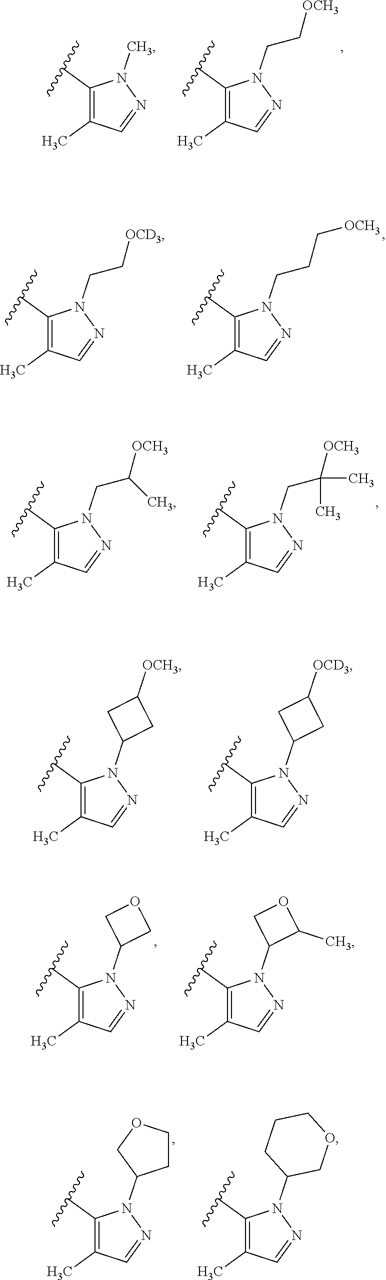

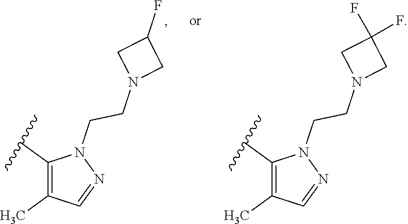 [SMILES: *c1c(C)cnn1CCN1CC(F)C1 ; *c1c(C)cnn1CCN1CC(F)(F)C1]

Provided herein as Embodiment 156 is the compound or salt of any one of Embodiments 146-148, wherein Z is

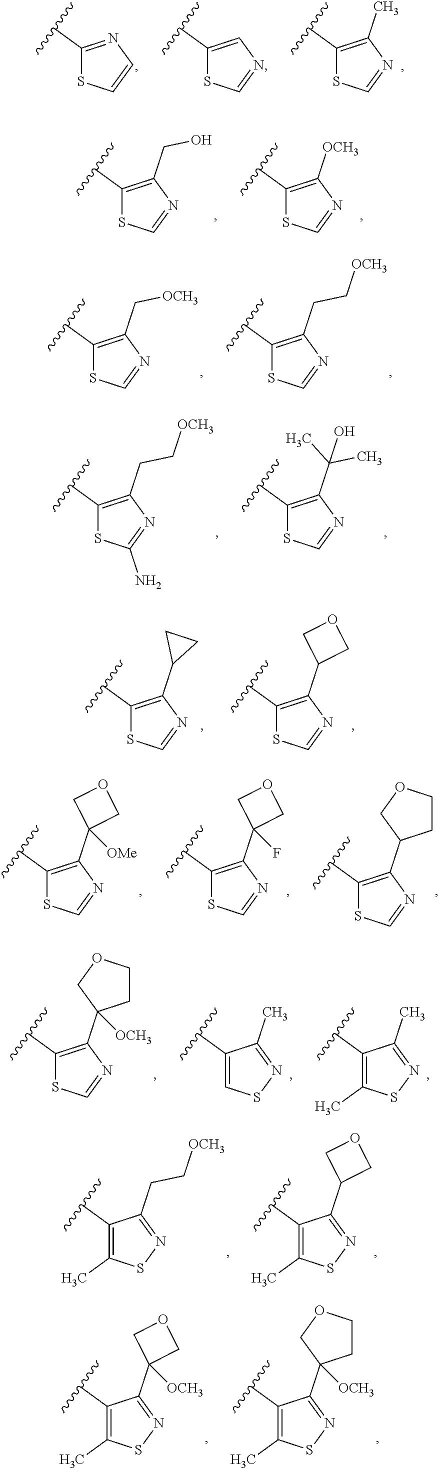

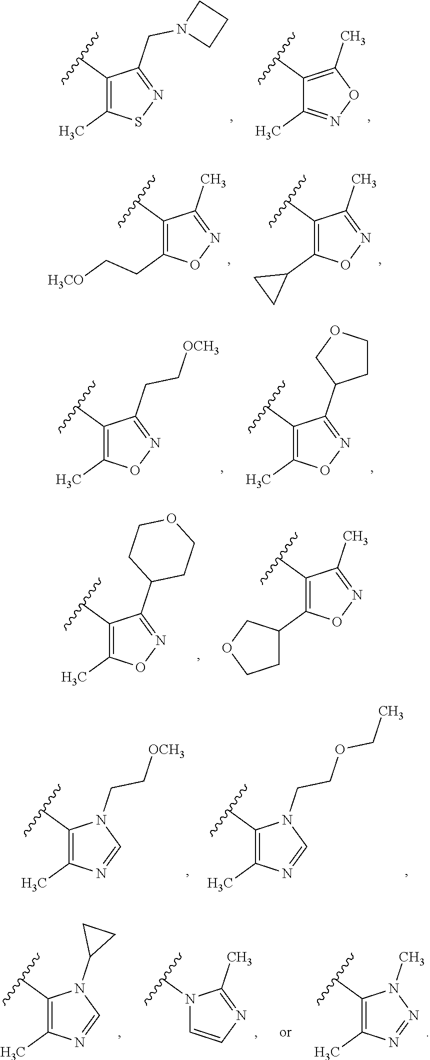

Provided herein as Embodiment 157 is the compound or salt of Embodiment 156, wherein Z is

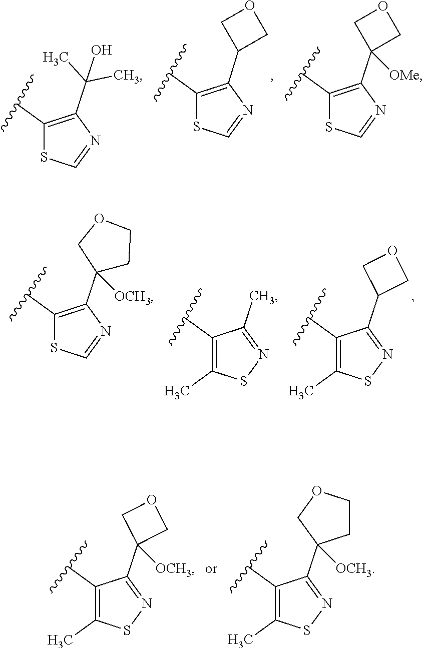

Provided herein as Embodiment 158 is the compound or salt of Embodiment 149, wherein Z is

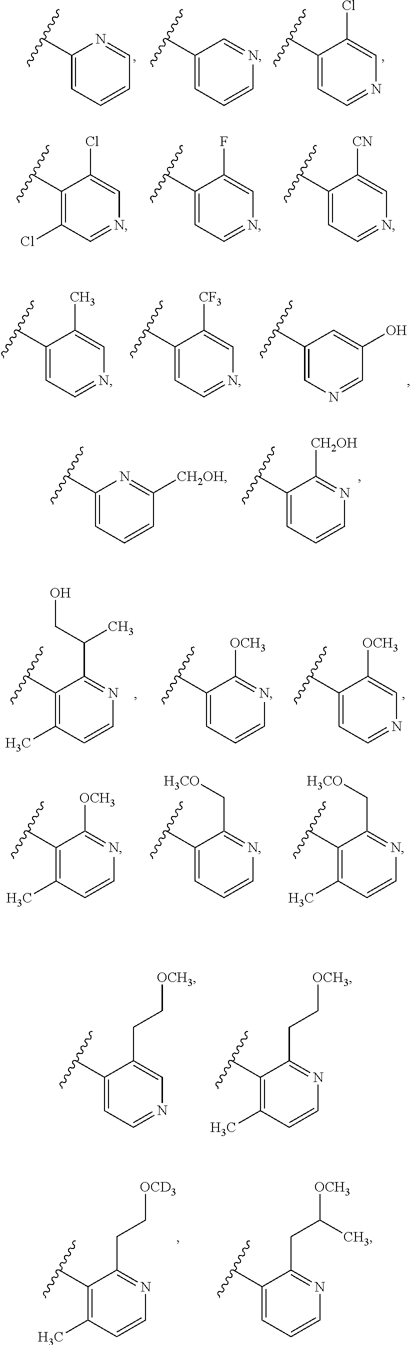

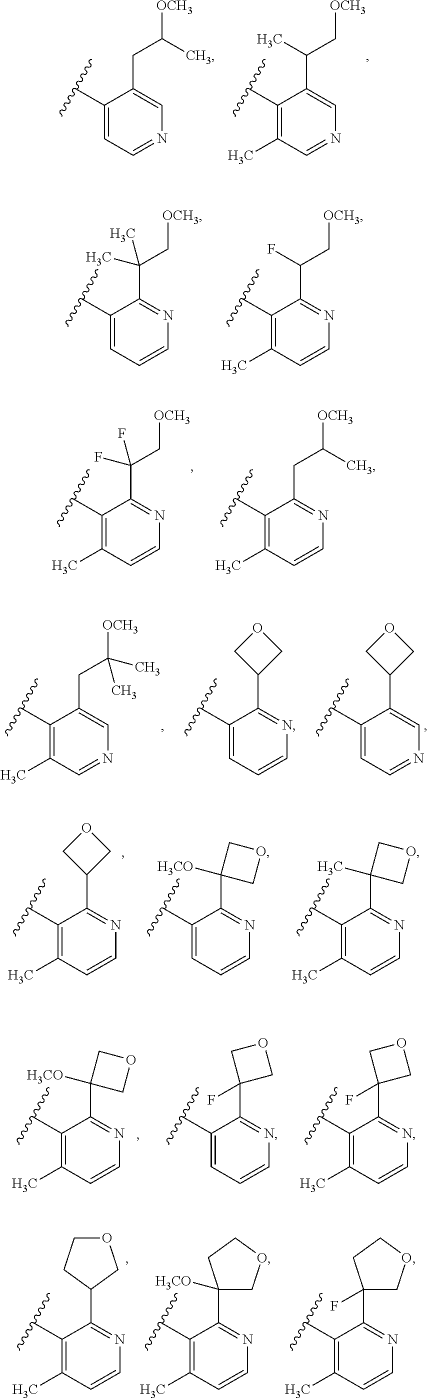

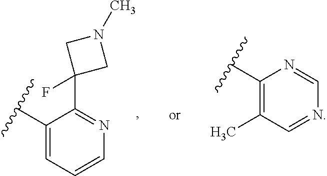

Provided herein as Embodiment 159 is the compound or salt of Embodiment 158, wherein Z is

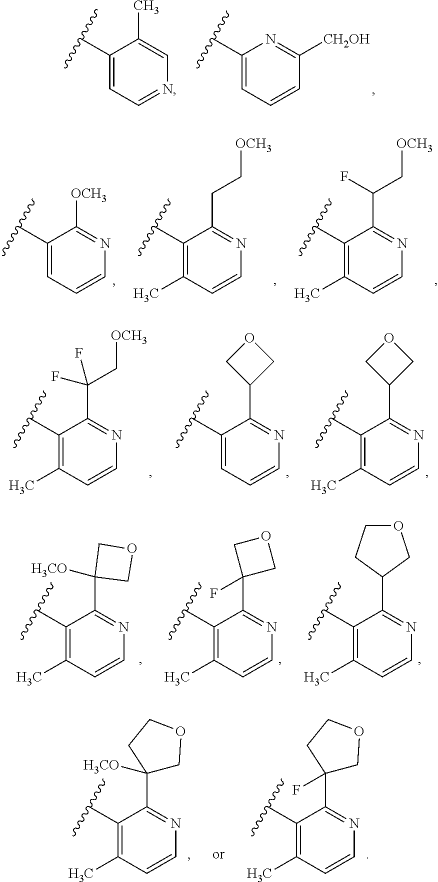

Provided herein as Embodiment 160 is the compound or salt of any one of Embodiments 1-142, wherein Z is a bicycle ring comprising a heteroaryl ring having 5 or 6 total ring atoms and 1-3 heteroatoms selected from N, O, and S fused to a cycloalkyl ring having 5 or 6 total ring atoms or a heterocycloalkyl ring having 5 or 6 total ring atoms and 1 or 2 heteroatoms selected from N, O, and S; wherein the bicyclic ring is optionally substituted with 1-4 substituents.

Provided herein as Embodiment 161 is the compound or salt of Embodiment 160, wherein the heteroaryl ring is pyridyl, pyridazinyl, pyrimidinyl, or pyrazinyl; the cycloalkyl ring is cyclopentyl or cyclohexyl; and the heterocycloalkyl ring is pyrrolidinyl, tetrahydrofuranyl, tetrahydropyranyl, or tetrahydrothiophenyl.

Provided herein as Embodiment 162 is the compound or salt of Embodiment 160 or 161, wherein the bicyclic ring is substituted with 1-4 substituents selected from halo, CN, C₁₋₆alkyl, C₁₋₆haloalkyl, C₀₋₃alkylene-OH, and C₀₋₂alkylene-C₁₋₃alkoxy.

Provided herein as Embodiment 163 is the compound or salt of any one of Embodiments 160-162, wherein Z is

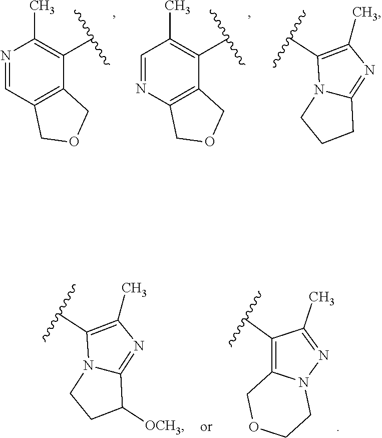 [SMILES: *c1c(C)ncc2c1COC2 ; *c1c(C)cnc2c1COC2 ; *c1c(C)nc2n1CCC2 ; *c1c(C)nc2n1CCC2OC ; *c1c(C)nn2c1COCC2]

Provided herein as Embodiment 164 is the compound or salt of Embodiment 1, wherein:

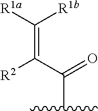

is

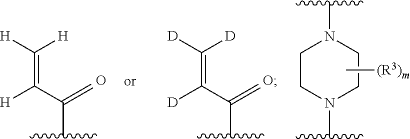

is

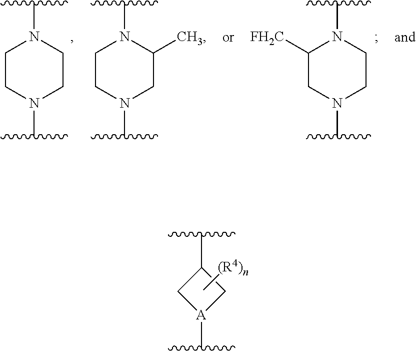

is

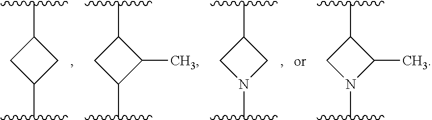

Provided herein as Embodiment 165 is the compound of Embodiment 164, wherein

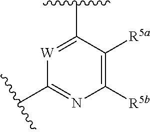

is

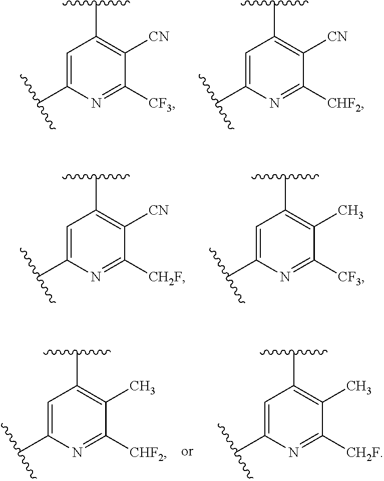

Provided herein as Embodiment 166 is the compound or salt of Embodiment 164 or 165, wherein X is

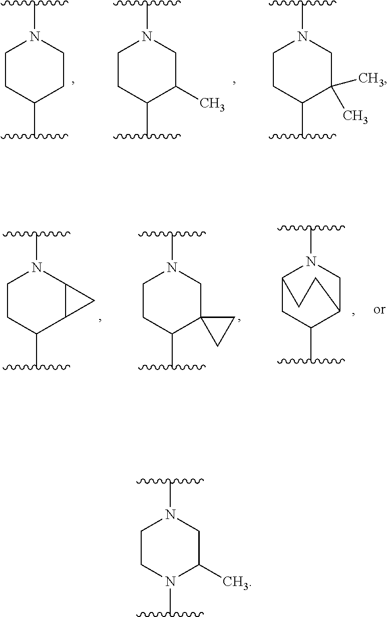

Provided herein as Embodiment 167 is the compound or salt of any one of Embodiments 164-166, wherein Z is

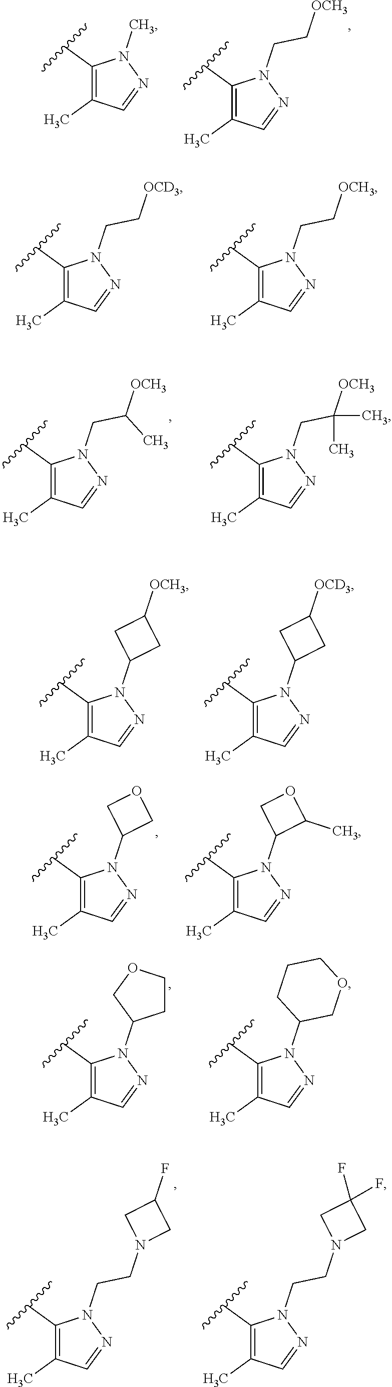

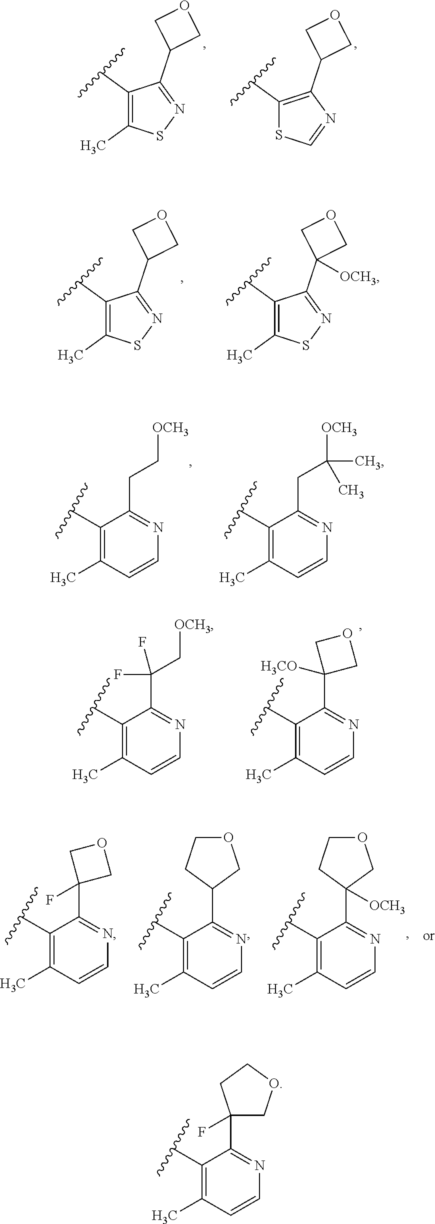

Provided herein as Embodiment 168 is the compound or salt of Embodiment 1, wherein the compound is a compound of:
Formula (I′):

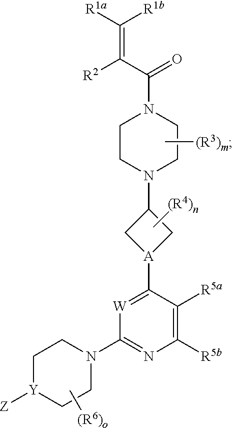

Formula (IA):

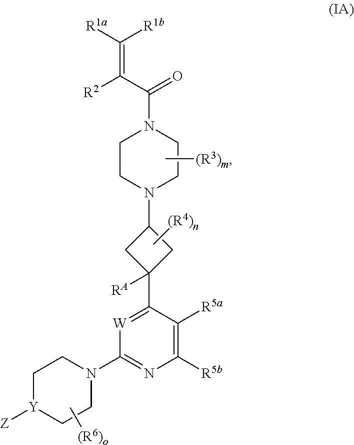

wherein R^{A} is H, halo, CN, C₁₋₃alkyl, C₁₋₃haloalkyl, C₀₋₆alkyleneOH, or C₀₋₆alkylene-C₁₋₃alkoxy; Formula (IB):

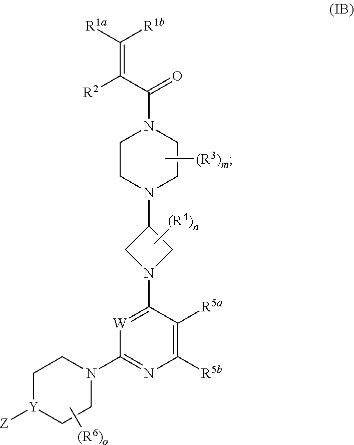

Formula (IC):

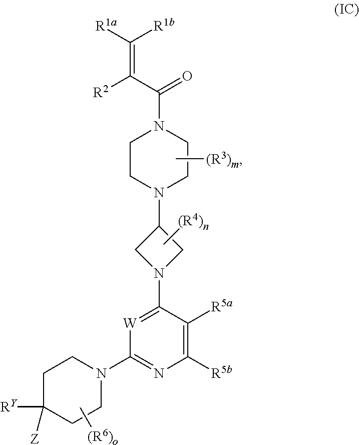

wherein
R^{Y} is H, halo, CN, C₁₋₃alkyl, C₁₋₃haloalkyl, C₀₋₃alkyleneOH, or C₀₋₆alkylene-C₁₋₄alkoxy; Formula (ID):

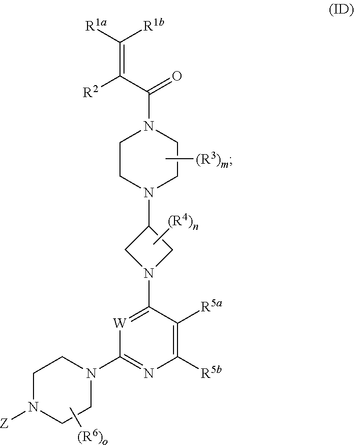

Formula (IE):

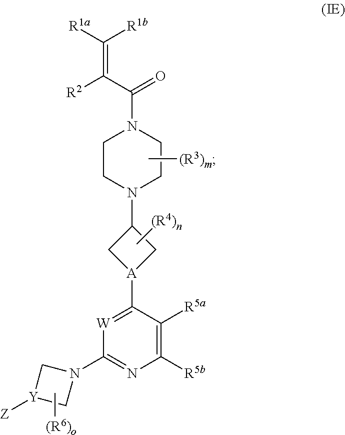

Formula (IF)

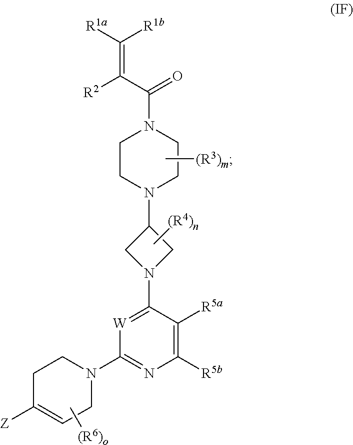

or Formula (IG)

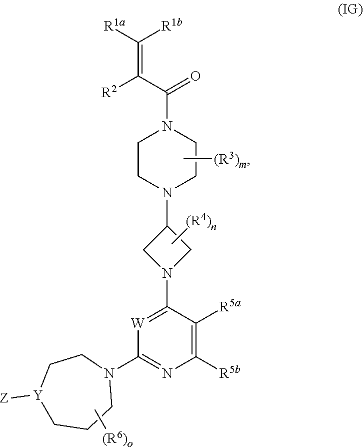

or a pharmaceutically acceptable salt of any of the foregoing.

Provided herein as Embodiment 169 is the compound of Embodiment 1, wherein the compound is

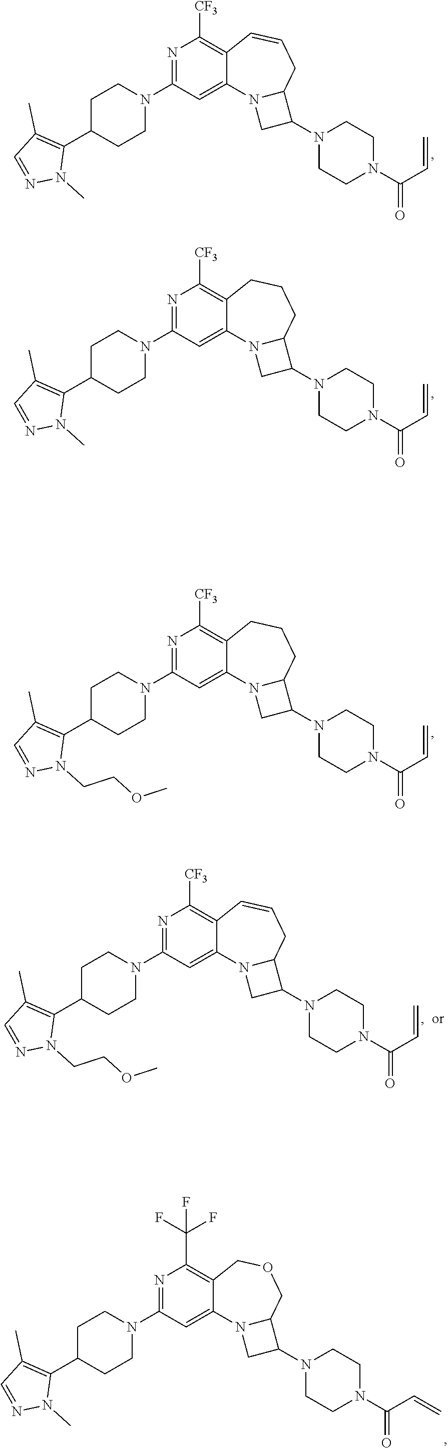 [SMILES: C=CC(=O)N1CCN(C2CN3c4cc(N5CCC(c6c(C)cnn6C)CC5)nc(C(F)(F)F)c4C=CCC23)CC1 ; C=CC(=O)N1CCN(C2CN3c4cc(N5CCC(c6c(C)cnn6C)CC5)nc(C(F)(F)F)c4CCCC23)CC1 ; C=CC(=O)N1CCN(C2CN3c4cc(N5CCC(c6c(C)cnn6CCOC)CC5)nc(C(F)(F)F)c4CCCC23)CC1 ; C=CC(=O)N1CCN(C2CN3c4cc(N5CCC(c6c(C)cnn6CCOC)CC5)nc(C(F)(F)F)c4C=CCC23)CC1 ; C=CC(=O)N1CCN(C2CN3c4cc(N5CCC(c6c(C)cnn6C)CC5)nc(C(F)(F)F)c4COCC23)CC1]

or a pharmaceutically acceptable salt thereof.

Provided herein as Embodiment 170 is the compound of Embodiment 1, wherein the compound is a compound listed in Table E, or a pharmaceutically acceptable salt thereof.

Provided herein as Embodiment 171 is the pharmaceutical composition comprising the compound or salt of any one of Embodiments 1-170 and a pharmaceutically acceptable excipient.

Provided herein as Embodiment 172 is the compound or salt of any one of Embodiments 1-170, or the pharmaceutical composition of Embodiment 171 for use as a medicament.

Provided herein as Embodiment 173 is the compound or salt of any one of Embodiments 1-170 or the pharmaceutical composition of Embodiment 171 for use in treating cancer.

Provided herein as Embodiment 174 is the compound or salt of any one of Embodiments 1-170 or the pharmaceutical composition of Embodiment 170 for use in treating cancer, wherein one or more cancer cells express KRAS G12C mutant protein.

Provided herein as Embodiment 175 is the compound, salt, or pharmaceutical composition for use of Embodiment 173 or 174, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, or a solid tumor.

Provided herein as Embodiment 176 is the use of a compound or salt of any one of Embodiments 1-170 or the pharmaceutical composition of Embodiment 171 in the preparation of a medicament for treating cancer.

Provided herein as Embodiment 177 is the use of a compound or salt of any one of Embodiments 1-170 or the pharmaceutical composition of Embodiment 171 in the preparation of a medicament for treating cancer, wherein one or more cancer cells express KRAS G12C mutant protein.

Provided herein as Embodiment 178 is the use of Embodiment 176 or 177, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, or a solid tumor.

Provided herein as Embodiment 179 is a method of treating cancer in a subject in need thereof, the method comprising administering to the subject a therapeutically effective amount of the compound or salt of any one of Embodiments 1-170, or the pharmaceutical composition of Embodiment 171.

Provided herein as Embodiment 180 is the method of Embodiment 179, wherein one or more cancer cells express KRAS G12C mutant protein.

Provided herein as Embodiment 181 is the method of Embodiment 179 or 180, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, or a solid tumor.

Provided herein as Embodiment 182 is the method of Embodiment 181, wherein the cancer is non-small cell lung cancer, colorectal cancer, pancreatic cancer, appendiceal cancer, endometrial cancer, esophageal cancer, cancer of unknown primary, ampullary cancer, gastric cancer, small bowel cancer, sinonasal cancer, bile duct cancer, melanoma, or a solid tumor.

Provided herein as Embodiment 183 is the method of Embodiment 182, wherein the cancer is non-small cell lung cancer.

Provided herein as Embodiment 184 is the method of Embodiment 182, wherein the cancer is colorectal cancer.

Provided herein as Embodiment 185 is the method of Embodiment 182, wherein the cancer is pancreatic cancer.

Provided herein as Embodiment 186 is the method of Embodiment 182, wherein the cancer is solid tumor.

Provided herein as Embodiment 187 is the method according to any one of Embodiments 179-186, wherein the subject has a cancer that was determined to have one or more cancer cells expressing the KRAS G/2C mutant protein prior to administration of the compound, salt, or pharmaceutical composition.

Provided herein as Embodiment 188, is the method according to any one of Embodiments 179-187, further comprising simultaneous, separate, or sequential administration of an effective amount of a second compound, wherein the second compound is an ATR inhibitor, Aurora kinase A inhibitor, AKT inhibitor, arginase inhibitor, CDK2 inhibitor, CDK4/6 inhibitor, ErbB family inhibitor, ERK inhibitor, FAK inhibitor, FGFR inhibitor, glutaminase inhibitor, IGF-IR inhibitor, KIF18A inhibitor, MAT2A inhibitor, MCL-1 inhibitor, MEK inhibitor, mTOR inhibitor, PARP inhibitor, PD-1 inhibitor, PD-L1 inhibitor, PI3K inhibitor, PRMT5 inhibitor, Raf kinase inhibitor, SHP2 inhibitor, SOS1 inhibitor, Sre kinase inhibitor, or one or more chemotherapeutic agents.

Provided herein as Embodiment 189 is the compound or salt of any one of Embodiments 1-170, wherein the compound or salt has an IC₅₀ value of less than 1 μM in the coupled exchange assay.

### Alternative Embodiments

Provided herein as Embodiment 1 is a compound of Formula (I):

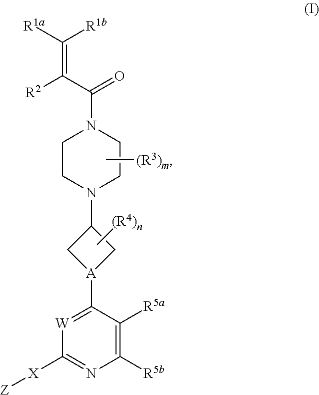

or a pharmaceutically acceptable salt thereof,
wherein:
m is 0, 1, 2, 3, or 4;
n is 1 or 2;
is 0, 1, 2, 3, or 4;
A is N, CH, C-halo, C—CN, C₁₋₃alkyl, C₁₋₃haloalkyl, C—C₀₋₀alkyleneOH, or C—C₀₋₃alkylene-C₁₋₄alkoxy;
W is CH, C-halo, C—CN, C—C₁₋₃alkyl, C—C₁₋₃haloalkyl, C—C₀₋₃alkyleneOH, or C—C₀₋₃alkylene-C₁₋₄alkoxy;
X is

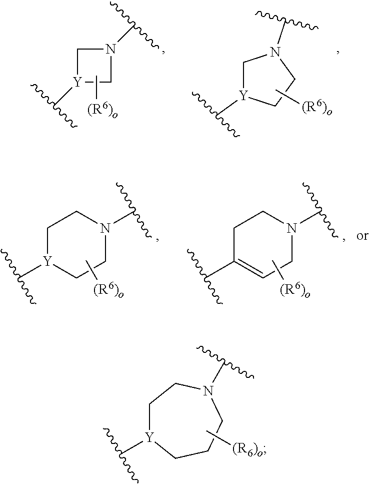

Y is N, C—H. C-halo, C—CN, C—C₁₋₃alkyl, C—C₁₋₃haloalkyl, C—C₀₋₃alkyleneOH, or C—C₀₋₂alkylene-Ct-alkoxy;
Z is phenyl, heteroaryl comprising 5 or 6 total ring atoms and 1-3 heteroatoms selected from N, O, and S, or a bicyclic ring comprising a heteroaryl ring having 5 or 6 total ring atoms and 1-3 heteroatoms selected from N, O, and S fused to C₅₋₆cycloalkyl or a heterocycloalkyl ring having 5 or 6 total ring atoms and 1 or 2 heteroatoms selected from N, O, and S; wherein each of the phenyl, heteroaryl, and bicyclic rings is optionally substituted with 1-4 substituents;
each of R^{1a}, R^{1b}, and R² independently is H, D, halo, C₁₋₄alkyl, C₁₋₄haloalkyl, C₁₋₂alkylene-OH, C₀₋₂alkylene-C₁₋₄alkoxy, C₀₋₂alkylene-C₁₋₄haloalkoxy, C₀₋₂alkylene-CN, C₀₋₂alkylene-N(R^{N1})₂, C₁₋₂alkylene-heterocycloalkyl having 3-6 total ring atoms and 1 or 2 heteroatoms selected from N, O, and S, or R^{1b} and R², together with the carbon atoms to which they are attached, from a

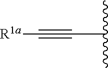

group;
each R³ independently is C₁₋₃alkyl, C₁₋₃haloalkyl, C₀₋₃alkyleneCN, C₀₋₃alkyleneOH, C₀₋₃alkylene-C₁₋₃alkoxy, oxo, spiro-cycloalkyl having 3-7 total ring atoms, spiro-heterocycloalkyl having 3-7 total ring atoms and 1 or 2 heteroatoms selected from N, O and S, or two adjacent R³, together with the atoms to which they are attached, form a fused cycloalkyl ring having 3-7 total ring atoms;
one R⁴ and R^{5a}, together with the atoms to which they are attached, form an optionally substituted ring having 6-10 total ring atoms and 0, 1, or 2 heteroatoms selected from N, O, and S, wherein the ring is saturated or unsaturated;
when n is 2, the other R⁴ is C₁₋₃alkyl, C₁₋₃haloalkyl, C₀₋₂alkyleneCN, C₁₋₂alkyleneOH, C₁₋₃alkylene-C₁₋₃alkoxy, oxo, spiro-cycloalkyl having 3-7 total ring atoms, or spiro-heterocycloalkyl having 4-7 total ring atoms and 1 or 2 heteroatoms selected from N, O and S;
R^{5b} is C₁₋₃haloalkyl, C₁₋₄alkyl, C₂₋₃alkenyl, C₁₋₃alkynyl, halo, C₁₋₃alkoxy, C₁₋₃thioalkoxy, cycloalkyl having 3-7 total ring atoms, cycloalkenyl having 5-7 total ring atoms, heterocycloalkyl having 3-7 total ring atoms and 1-3 heteroatoms selected from N, O, and S, or heterocycloalkenyl having 5-7 total ring atoms and 1-3 heteroatoms selected from N, O, and S, wherein each of foregoing is independently optionally substituted with 1-3 substituents;
each R⁶ independently is halo, CN, oxo, C₁₋₃alkyl, C₁₋₃haloalkyl, C₀₋₆alkyleneOH, C₀₋₃alkylene-C₁₋₃alkoxy, deuterated C₀₋₃alkylene-C₁₋₃alkoxy, C₁₋₄alkylene-N(R^{N1})₂, spiro-cycloalkyl having 3-7 total ring atoms, spiro-heterocycloalkyl having 4-7 total ring atoms and 1 or 2 heteroatoms selected from N, O and S; or two adjacent R⁶, together with the atoms to which they are attached, form a fused cycloalkyl ring having 3-7 total ring atoms; or Y and an adjacent R⁶, together with the atoms to which they are attached, form a fused cycloalkyl ring having 3-7 total ring atoms; wherein the fused cycloalkyl ring of any of the foregoing is optionally substituted with 1 or 2 substituents; or
two non-adjacent R⁶ join together to form a C₁₋₃alkylene bridge or a C₁₋₃ether bridge; and
Provided herein as Embodiment 2 is the compound or salt of Embodiment 1, wherein

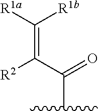

is

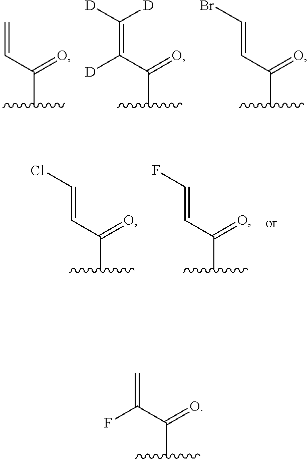

Provided herein as Embodiment 3 is the compound or salt of Embodiment 1 or 2, wherein

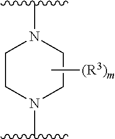

is

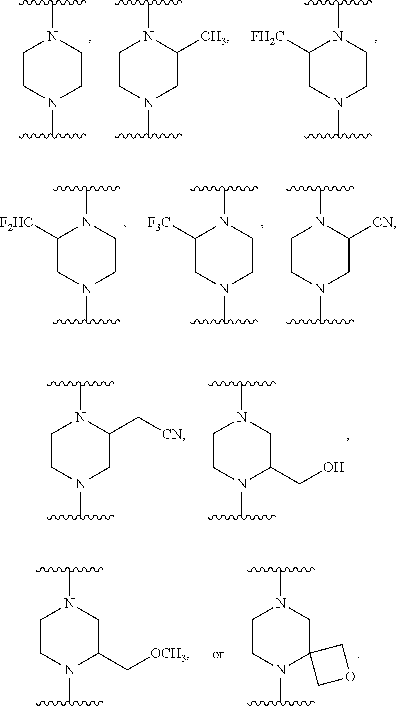

Provided herein as Embodiment 4 is the compound or salt of any one of Embodiments 1-3, wherein A is N.

Provided herein as Embodiment 5 is the compound or salt of any one of Embodiments 1-4, wherein n is 1.

Provided herein as Embodiment 6 is the compound or salt of any one of Embodiments 1-4, wherein n is 2.

Provided herein as Embodiment 7 is the compound or salt of Embodiment 6, wherein the other R⁴ is CH₃, CH₂CH₃, CH₂CH₂CH₃, CH(CH₃)₂, CF₃, CHF₂, or CH₂F.

Provided herein as Embodiment 8 is the compound or salt of any one of Embodiments 1-7, wherein W is CH.

Provided herein as Embodiment 9 is the compound or salt of any one of Embodiments 1-8, wherein one R⁴ and R^{5a}, together with the atoms to which they are attached, form an optionally substituted ring having 6-10 total ring atoms and 0, 1, or 2 heteroatoms selected from N, O, and S, wherein the ring is saturated.

Provided herein as Embodiment 10 is the compound or salt of any one of Embodiments 1-8, wherein one R⁴ and R^{5a}, together with the atoms to which they are attached, form an optionally substituted ring having 6-10 total ring atoms and 0, 1, or 2 heteroatoms selected from N, O, and S, wherein the ring is unsaturated.

Provided herein as Embodiment 11 is the compound or salt of any one of Embodiments 1-10, wherein the optionally substituted ring formed by one R⁴ and R^{5a}, together with the atoms to which they are attached, has 6 total ring atoms.

Provided herein as Embodiment 12 is the compound or salt of any one of Embodiments 1-10, wherein the optionally substituted ring formed by one R⁴ and R^{5a}, together with the atoms to which they are attached, has 7 total ring atoms.

Provided herein as Embodiment 13 is the compound or salt of any one of Embodiments 1-10, wherein the optionally substituted ring has 8 total ring atoms.

Provided herein as Embodiment 14 is the compound or salt of any one of Embodiments 1-10, wherein the optionally substituted ring formed by one R⁴ and R^{5a}, together with the atoms to which they are attached, has 9 or 10 total ring atoms.

Provided herein as Embodiment 15 is the compound or salt of any one of Embodiments 1-14, wherein the optionally substituted ring formed by one R⁴ and R^{1a}, together with the atoms to which they are attached, has 0 heteroatoms.

Provided herein as Embodiment 16 is the compound or salt of any one of Embodiments 1-14, wherein the optionally substituted ring formed by one R⁴ and R^{5a}, together with the atoms to which they are attached, has 1 or 2 heteroatoms selected from N, O, and S.

Provided herein as Embodiment 17 is the compound or salt of Embodiment 16, wherein the 1 or 2 heteroatoms are each O.

Provided herein as Embodiment 18 is the compound or salt of Embodiment 17, wherein the optionally substituted ring is an ether.

Provided herein as Embodiment 19 is the compound or salt of Embodiment 16, wherein the 1 or 2 heteroatoms are each N.

Provided herein as Embodiment 20 is the compound or salt of Embodiment 19, wherein the ring is a lactam or a cyclic amine.

Provided herein as Embodiment 21 is the compound or salt of any one of Embodiments 1-20, wherein the ring formed by one R⁴ and R^{5a}, together with the atoms to which they are attached, is unsubstituted.

Provided herein as Embodiment 22 is the compound or salt of any one of Embodiments 1-20, wherein the ring formed by one R⁴ and R^{5a}, together with the atoms to which they are attached, is substituted with 1 or 2 substituents selected from the group consisting of C₁₋₃alkyl, C₁₋₃haloalkyl, oxo, halo, CN, C₀₋₃alkyleneOH, C₀₋₆alkylene-C₁₋₃alkoxy, cycloalkyl having 3-7 total ring atoms, cycloalkenyl having 5-7 total ring atoms, heterocycloalkyl having 4-7 total ring atoms and 1-3 heteroatoms selected from N, O, and S, heterocycloalkenyl having 5-7 total ring atoms and 1-3 heteroatoms selected from N, O, and S, and phenyl.

Provided herein as Embodiment 23 is the compound or salt of Embodiment 1, wherein

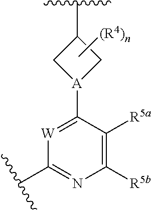

is

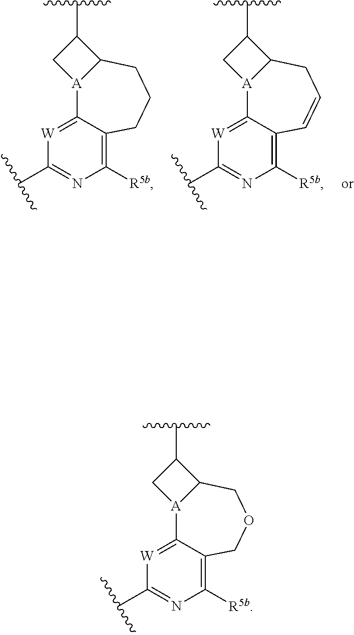

Provided herein as Embodiment 24 is the compound or salt of any one of Embodiments 1-23, wherein R^{5b} is CF₃, CF H, CFH₂, or CF CH₃.

Provided herein as Embodiment 25 is the compound or salt of any one of Embodiments 1-24, wherein X is

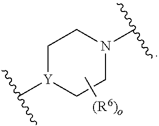

Provided herein as Embodiment 26 is the compound or salt of any one of Embodiment 1-25, wherein Y is C—H.

Provided herein as Embodiment 27 is the compound or salt of any one of Embodiments 1-26, wherein o is 0.

Provided herein as Embodiment 28 is the compound or salt of any one of Embodiments 1-26, wherein o is 1.

Provided herein as Embodiment 29 is the compound or salt of Embodiment 28, wherein R⁶ is CH₃, CH₂F, CHF₂, or CF₃.

Provided herein as Embodiment 30 is the compound or salt of Embodiment 25, wherein

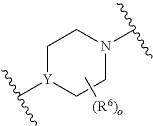

is

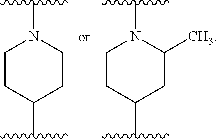

Provided herein as Embodiment 31 is the compound or salt of any one of Embodiments 1-30, wherein Z is heteroaryl comprising 5 or 6 total ring atoms and 1-3 heteroatoms selected from N, O, and S, wherein the heteroaryl is optionally substituted with 1-4 substituents.

Provided herein as Embodiment 32 is the compound or salt of Embodiment 31, wherein the heteroaryl is pyrrolyl, furanyl, thiophenyl, pyrazolyl, imidazolyl, thiazolyl, isothiazolyl, oxazolyl, isoxazolyl, triazolyl, thiadiazolyl, oxadiazolyl, pyridyl, pyridazinyl, pyrimidinyl, pyrazinyl, or triazinyl.

Provided herein as Embodiment 33 is the compound or salt of Embodiment 32, wherein the heteroaryl is pyrazolyl or pyridyl.

Provided herein as Embodiment 34 is the compound or salt of any one of Embodiments 31-33, wherein the heteroaryl is substituted with 1-4 substituents, each of which independently is selected from the group consisting of halo, CN, C₁₋₃alkyl, C₁₋₃haloalkyl, C₂₋₆alkenyl, C₂₋₆haloalkenyl, C₀₋₆alkylene-OH, C₀₋₆alkylene-C₁₋₃alkoxy, C₀₋₆alkylene-N(R^{N1})₂, wherein each R^{N1} independently is H or C₁₋₃alkyl, C₀₋₂alkylene-cycloalkyl having 3-6 total ring atoms, C₀₋₂alkylene-heterocycloalkyl having 5 or 6 total ring atoms and 1-3 heteroatoms selected from N, O, and S, and C₀₋₂alkylene-phenyl; wherein each of the alkyl, alkenyl, C₀₋₆alkylene-C₁₋₃alkoxy, cycloalkyl, heterocycloalkyl, and phenyl substituents independently is optionally substituted with 1-3 substituents independently selected from deuterium, halo, OH, CH₃, OCH₃, and OCD₃.

Provided herein as Embodiment 35 is the compound or salt of Embodiment 34, wherein each of the 1-4 substituents independently is selected from the group consisting of Cl, F, CN, CH₃, CD₃, CH₂CH₃, CH(CH₃)₂, CF₃, CHF₂, CH₂F, CH₂CHF₂, CH₂CH₂F, CH(CH₂F) 2, CH(CH₃)CH₂F, CH(CH)CHF₂, C(═CH)CHF, OH, CH₂OH, CH₂CH₂OH, CH(CH₃)CH₂OH, C(CH₃)₂OH, C(CH₃)₂CH₂OH, CH₂C(CH₃)₂OH, OCH₃, OCD₃, CH₂OCH₃, CH₂OCD₃, CH₂CH₂OCH₃, CHFCH₂OCH₃, CF₂CH₂OCH₃, CH₂CH₂OCD₃, CH₂CH₂OCH₂CH₃,CH₂CH₂CH₂OCH₃, CH₂CH₂CH₂OCD₃, CH(CH₃)CH₂OCH₃, CH(CH₃)CH₂OCD₃,C(CH₃)₂CH₂OCH₃, C(CH₃)₂CH₂OCD₃, CH₂CH(CH₃)OCH₃, CH₂CH(CH₃)OCD₃, CH₂C(CH₃)₂OCH; CH₂C(CH₃)₂OCD₃, NH₂, CH₂NH₂, CH₂NHCH₃, CH₂N(CH₃)₂, CH₂CH₂NH₂, CH₂CH₂NHCH₃, CH₂CH₂N(CH₃):

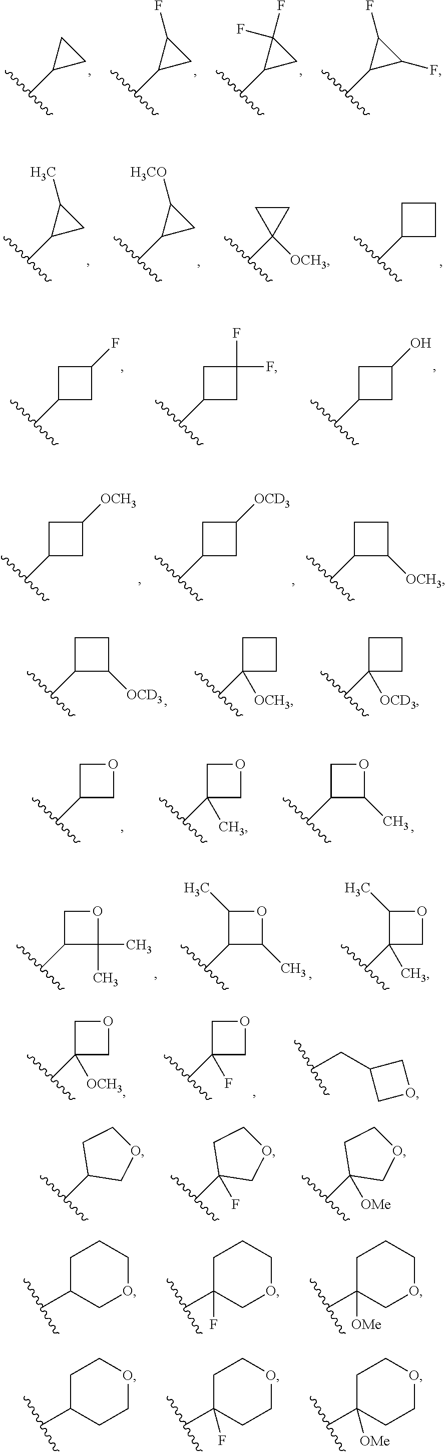

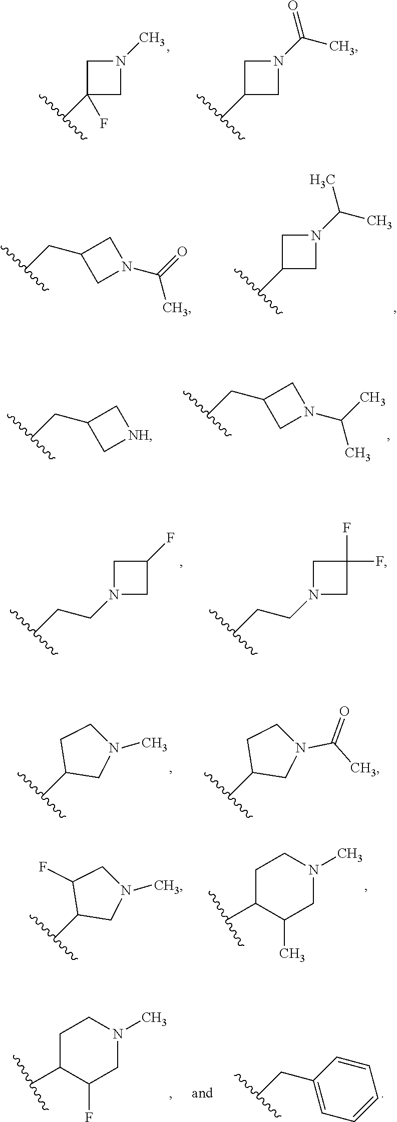

Provided herein as Embodiment 36 is the compound or salt of Embodiment 35, wherein each of the 1-4 substituents independently is CH₃, CH₂CH₂OCH₃, CH₂CH₂OCD₃,

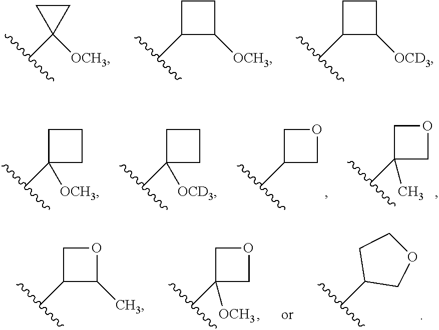

Provided herein as Embodiment 37 is the compound or salt of any one of Embodiments 31-33, wherein Z is

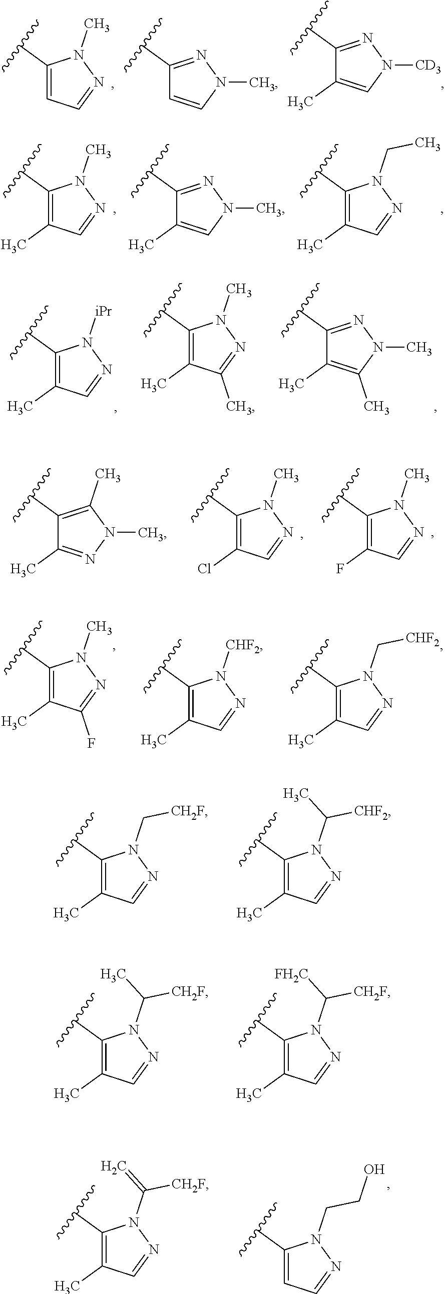

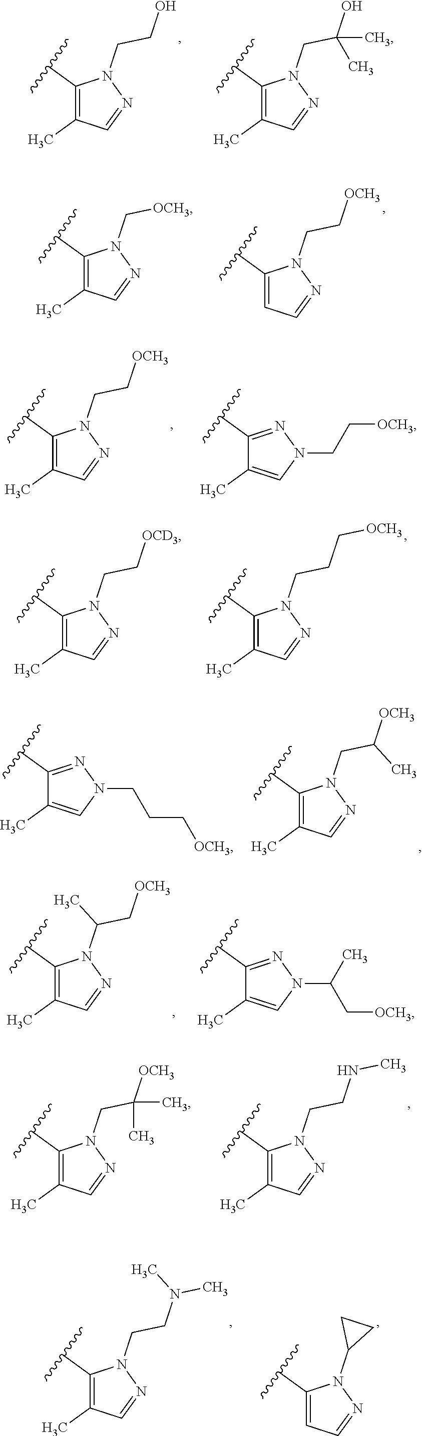

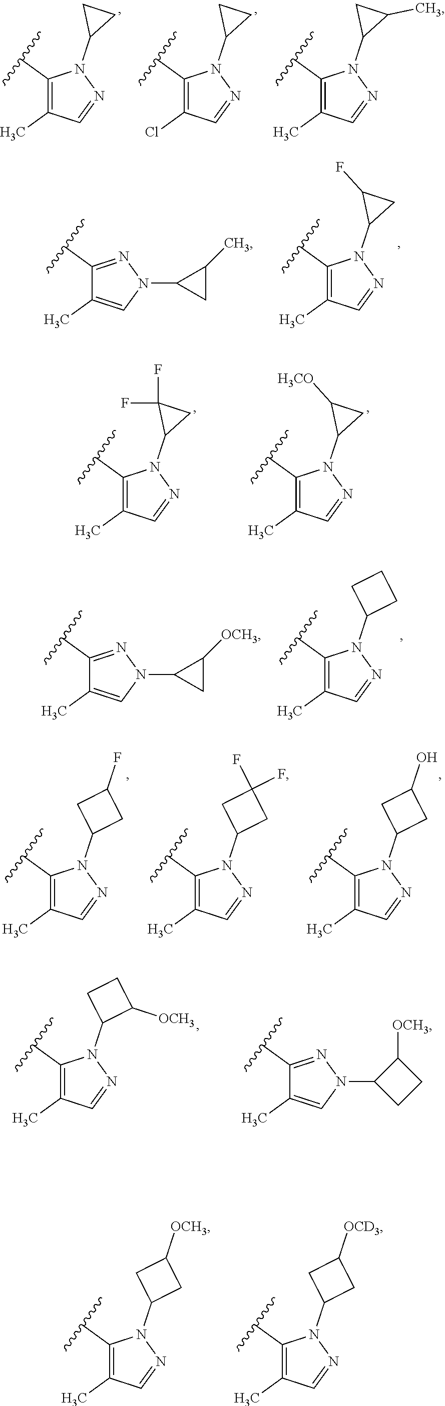

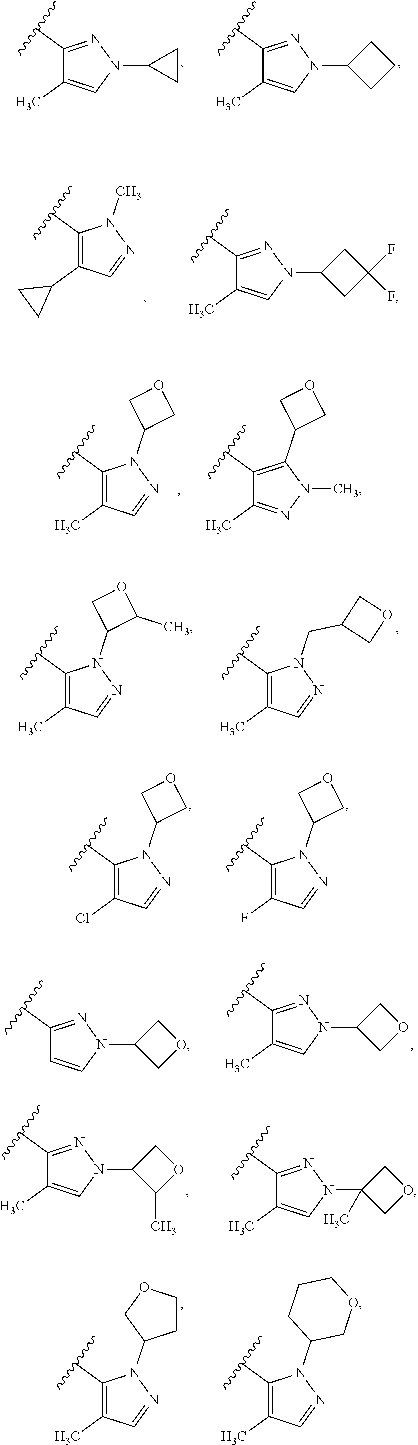

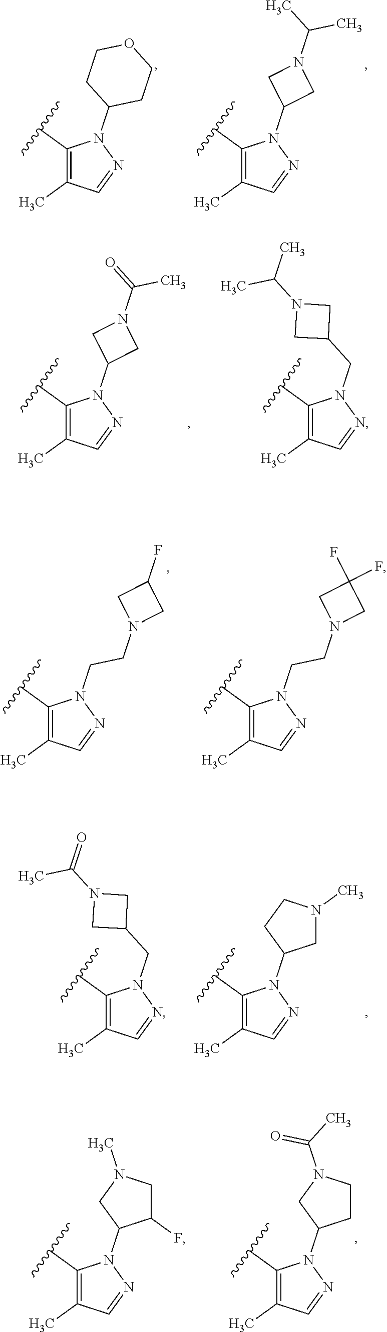

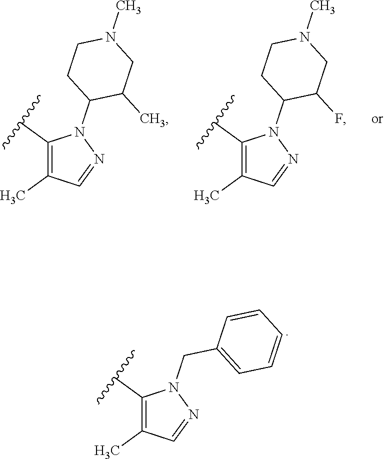 [SMILES: *c1c(C)cnn1C1CCN(C)CC1C ; *c1c(C)cnn1C1CCN(C)CC1F ; "or" ; *c1c(C)cnn1Cc1ccccc1]

Provided herein as Embodiment 38 is the compound or salt of Embodiment 37, wherein Z is

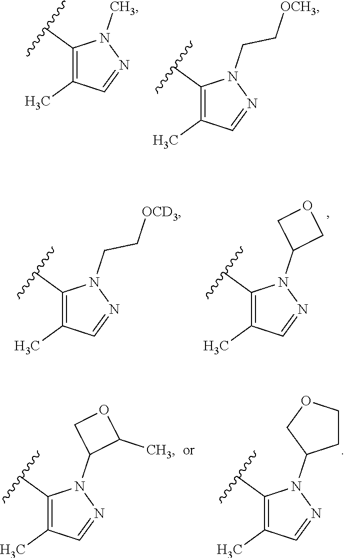

Provided herein as Embodiment 39 is the compound or salt of any one of Embodiments 31-33, wherein Z is

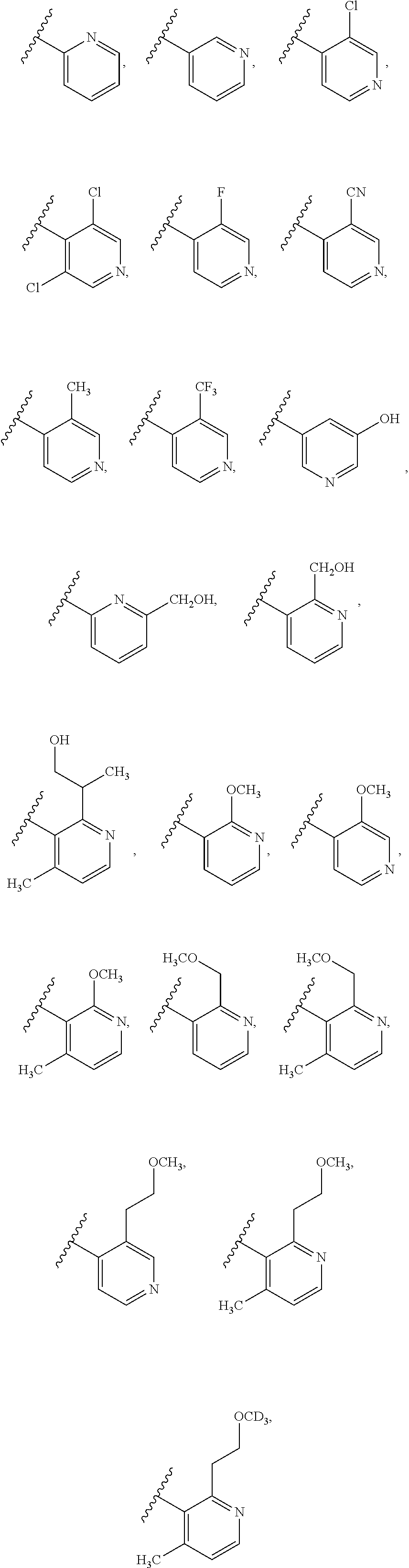

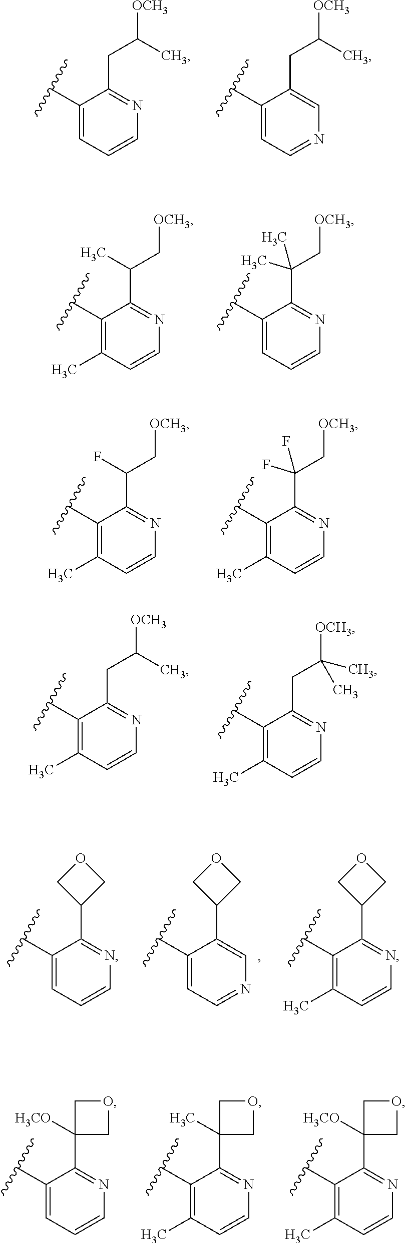

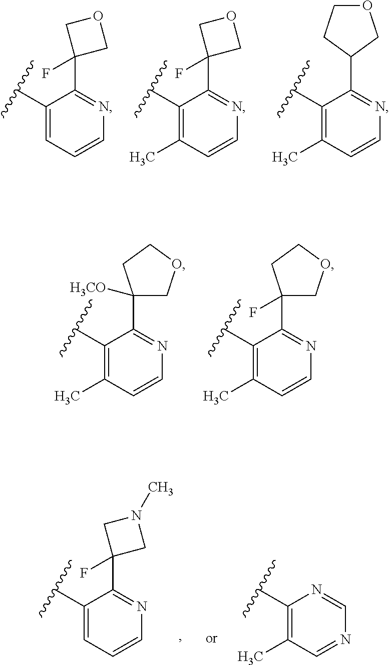

Provided herein as Embodiment 40 is the compound or salt of Embodiment 39, wherein Z is

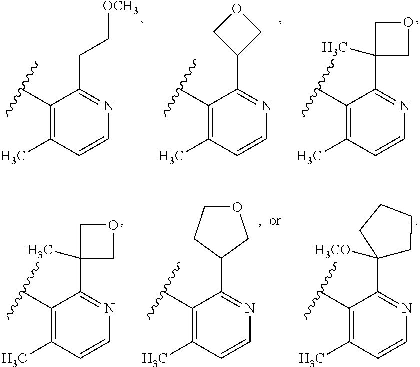 [SMILES: *c1c(C)ccnc1CCOC ; *c1c(C)ccnc1C1COC1 ; *c1c(C)ccnc1C1(C)COC1 ; *c1c(C)ccnc1C1(C)COC1 ; *c1c(C)ccnc1C1CCOC1 ; *c1c(C)ccnc1C1(OC)CCCC1]

Provided herein as Embodiment 41 is the compound or salt of Embodiment 1, wherein:

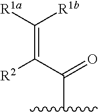

is

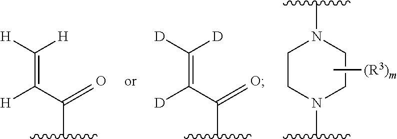

is

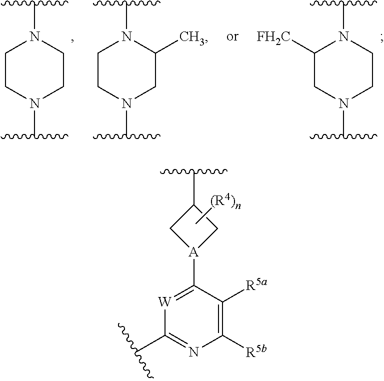

is

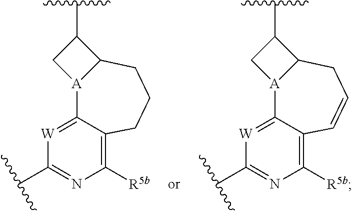

### X is

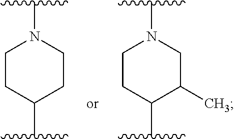 [SMILES: *C1CCN(*)CC1 ; *C1CCN(*)CC1C]

and Z is pyrazolyl or pyridyl, each of which is optionally substituted with 1-4 substituents.

Provided herein as Embodiment 42 is the compound or salt of Embodiment 41, wherein each of the 1-4 substituents of Z independently is CH₃, CH₂CH₂OCH₃, CH₂CH₂OCD₃,

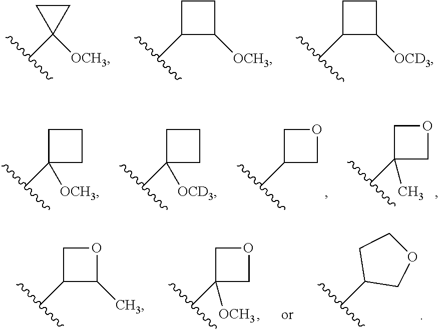

Provided herein as Embodiment 43 is the compound or salt of Embodiment 41 or 42, wherein Z is substituted with 2 substituents.

Provided herein as Embodiment 44 is the compound or salt of Embodiment 43, wherein at least one substituent is CH₃.

Provided herein as Embodiment 45 is the compound or salt of Embodiment 43, wherein each substituent is CH₃.

Provided herein as Embodiment 46 is the compound or salt of Embodiment 43 or 44, wherein Z is substituted with CH₃ and CH₂CH₂OCH₃.

Provided herein as Embodiment 47 is the compound or salt of Embodiment 43 or 44, herein Z is substituted with CH₃ and

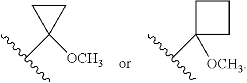 [SMILES: *C1(OC)CC1 ; *C1(OC)CCC1]

Provided herein as Embodiment 48 is the compound or salt of Embodiment 43 or 44, wherein Z is substituted with CH₃ and

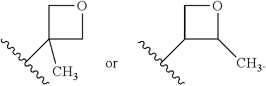

Provided herein as Embodiment 49 is the compound or salt of Embodiment 43 or 44, wherein Z is substituted with CH₃ and

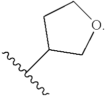 [SMILES: *C1CCOC1]

Provided herein as Embodiment 50 is the compound or salt of Embodiment 41, wherein Z is

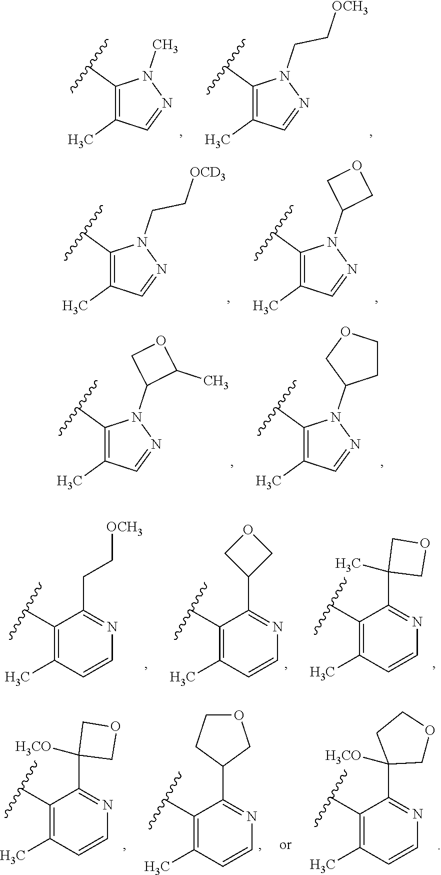

Provided herein as Embodiment 51 is the compound or salt of Embodiment 50, wherein Z is

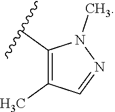 [SMILES: *c1c(C)cnn1C]

Provided herein as Embodiment 52 is the compound or salt of Embodiment 50, wherein Z is

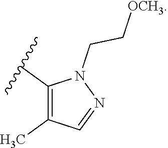 [SMILES: *c1c(C)cnn1CCOC]

Provided herein as Embodiment 53 is the compound or salt of Embodiment 46, wherein Z is

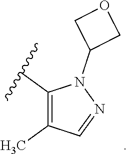 [SMILES: *c1c(C)cnn1C1COC1]

Provided herein as Embodiment 54 is the compound or salt of Embodiment 50, wherein Z is

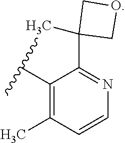 [SMILES: *c1c(C)ccnc1C1(C)COC1]

Provided herein as Embodiment 55 is the compound or salt of Embodiment 50, wherein Z is

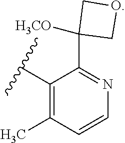 [SMILES: *c1c(C)ccnc1C1(OC)COC1]

Provided herein as Embodiment 56 is the compound or salt of Embodiment I having a structure:

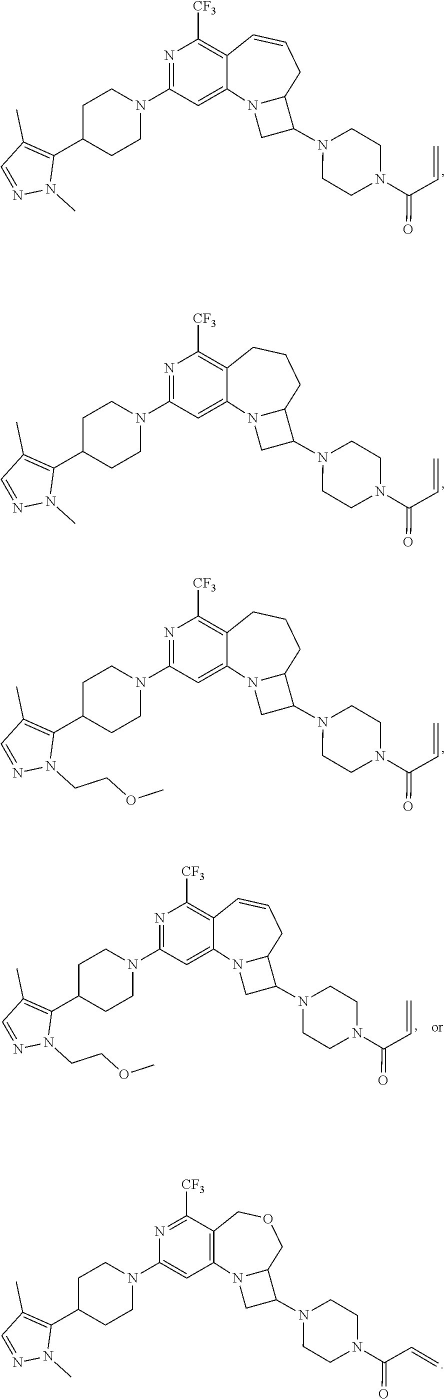 [SMILES: C=CC(=O)N1CCN(C2CN3c4cc(N5CCC(c6c(C)cnn6C)CC5)nc(C(F)(F)F)c4C=CCC23)CC1 ; C=CC(=O)N1CCN(C2CN3c4cc(N5CCC(c6c(C)cnn6C)CC5)nc(C(F)(F)F)c4CCCC23)CC1 ; C=CC(=O)N1CCN(C2CN3c4cc(N5CCC(c6c(C)cnn6CCOC)CC5)nc(C(F)(F)F)c4CCCC23)CC1 ; C=CC(=O)N1CCN(C2CN3c4cc(N5CCC(c6c(C)cnn6CCOC)CC5)nc(C(F)(F)F)c4C=CCC23)CC1 ; C=CC(=O)N1CCN(C2CN3c4cc(N5CCC(c6c(C)cnn6C)CC5)nc(C(F)(F)F)c4COCC23)CC1]

or a pharmaceutically acceptable salt thereof.

Provided herein as Embodiment 57 is the compound of Embodiment 56 having a structure:

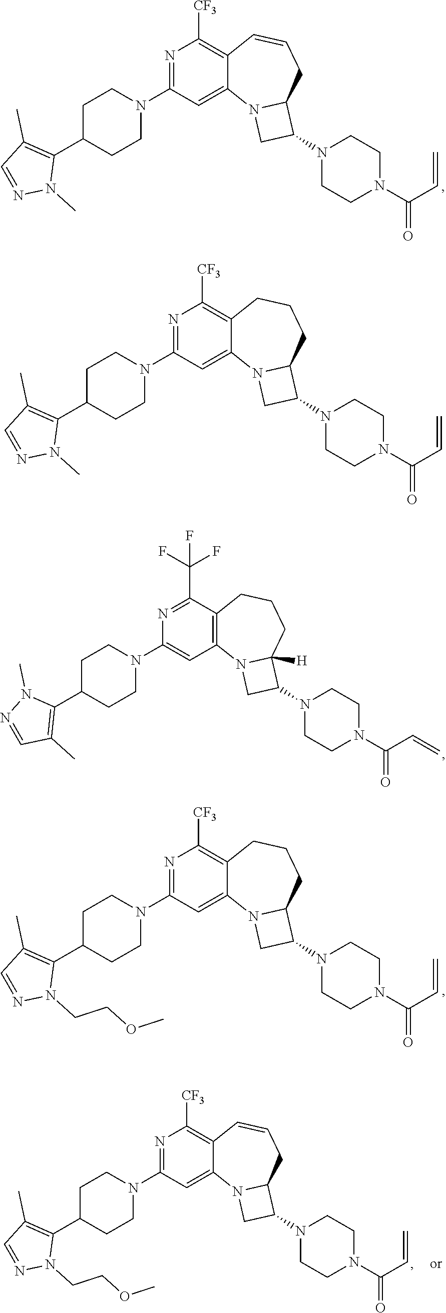

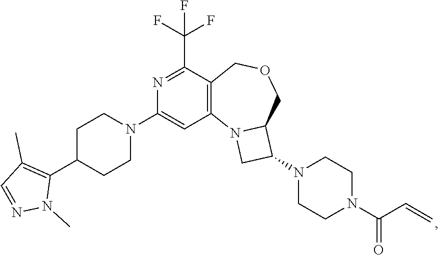 [SMILES: C=CC(=O)N1CCN([C@@H]2CN3c4cc(N5CCC(c6c(C)cnn6C)CC5)nc(C(F)(F)F)c4COC[C@@H]23)CC1]

or a pharmaceutically acceptable salt thereof.

Provided herein as Embodiment 58 is a compound of Formula (II):

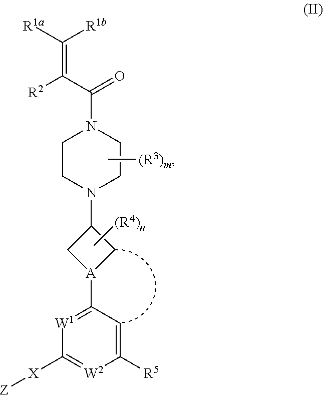

or a pharmaceutically acceptable salt thereof,
wherein:
m is 0, 1, 2, 3, or 4;
n is 0, 1, or 2;
A is N, CH, C-halo, C—CN, C—C₁₋₃alkyl, C—C₁₋₄haloalkyl, C—C₀₋₃alkyleneOH, or C—C₀₋₃alkylene-C₁₋₄alkoxy;
each of W¹ and W² independently is N, CH, C-halo, C—CN, C—C; alkyl, C—C₂₋₃alkenyl, C—C₂₋₃alkynyl, C—C₁₋₃haloalkyl, C—C₀₋₂alkyleneOH, or C—C₀₋₃alkylene-C₁₋₄alkoxy, wherein each of the alkenyl and alkynyl is unsubstituted or substituted with 1 or more substituents;
X is heterocycloalkyl or heterocycloalkenyl, each having 4-7 total ring atoms and 1-3 heteroatoms selected from N, O, and S, wherein each of the heterocycloalkyl and heterocycloalkenyl is unsubstituted or substituted with 1 or more substituents; Z is phenyl, heteroaryl comprising 5 or 6 total ring atoms and 1-3 heteroatoms selected from N, O, and S, or a bicyclic ring comprising a heteroaryl having 5 or 6 total ring atoms and 1-3 heteroatoms selected from N, O, and S fused to a to a cycloalkyl ring having 5 or 6 total ring atoms or a heterocycloalkyl ring having 5 or 6 total ring atoms and 1 or 2 heteroatoms selected from N, O, and S; wherein each of the phenyl, heteroaryl, and bicyclic ring is unsubstituted or substituted with 1 or more substituents;
[symbol] is C₂₋₆alkylene, C₂₋₆alkenylene, heteroalkylene having 2-6 total atoms and 1˜3 heteroatoms selected from N, O, and S, or heteroalkenylene having 3-6 total atoms and 1 or 2 heteroatoms selected from N, O, and S, wherein [symbol] is unsubstituted or substituted with 1 or more substituents;
each of R^{1a}, R^{1b}, and R² independently is H, D, halo, C₁₋₄alkyl, C₁₋₃haloalkyl, C₁₋₂alkylene-OH, C₀₋₂alkylene-C₁₋₄alkoxy, C₀₋₂alkylene-C₁₋₄haloalkoxy, C₀₋₂alkylene-CN, C₀₋₂alkylene-N(R^{N1})₂, C₁₋₂alkylene-heterocycloalkyl having 3-6 total ring atoms and 1 or 2 heteroatoms selected from N, O, and S, or R^{1b} and R², together with the carbon atoms to which they are attached, form

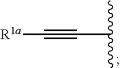

each R³ independently is C₁₋₃alkyl, C₁₋₃haloalkyl,

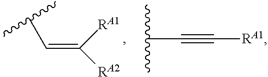

C₀₋₃alkyleneCN, C₀₋₃alkyleneOH, or C₀₋₆alkylene-C₁₋₃alkoxy; two geminal R³, together with the atom to which they are attached, form oxo, spiro-C₃₋₇cycloalkyl, spiro-C₄₋₇cycloalkenyl, spiro-heterocycloalkyl having 3-7 total ring atoms and 1 or 2 heteroatoms selected from N, O, and S, or spiro-heterocycloalkenyl having 4-7 total ring atoms and 1 or 2 heteroatoms selected from N, O, and S; or two vicinal R³, together with the atoms to which they are attached, form fused-C₃₋₇-cycloalkyl, fused-C₄₋₇cycloalkenyl, fused-heterocycloalkyl having 3-7 total ring atoms and 1 or 2 heteroatoms selected from N, O, and S, or fused-heterocycloalkenyl having 4-7 total ring atoms and 1 or 2 heteroatoms selected from N, O, and S;
each R⁴ independently is C₁₋₃alkyl, C₁₋₃haloalkyl, C₀₋₃alkyleneCN, C₁₋₃alkyleneOH, or C₁₋₃alkylene-C₁₋₃alkoxy; or two geminal R⁴, together with the atom to which they are attached, form oxo, spiro-C₃₋₇cycloalkyl, or spiro-heterocycloalkyl having 4-7 total ring atoms and 1 or 2 heteroatoms selected from N, O and S;
R⁵ is halo, C₁₋₃haloalkyl, C₁-6alkyl, C₂₋₄alkenyl, C₂₋₄alkynyl, C₁₋₃alkoxy, C₁₋₃thioalkyl, C₃₋₇cycloalkyl, C₅₋₇cycloalkenyl, heterocycloalkyl having 3-7 total ring atoms and 1-3 heteroatoms selected from N, O, and S, or heterocycloalkenyl having 5-7 total ring atoms and 1-3 heteroatoms selected from N, O, and S, wherein each of the foregoing independently is unsubstituted or substituted with 1 or more substituents;
each of R^{A1} and R^{A2} independently is H, C₁₋₃alkyl, C₁₋₃haloalkyl, or C₃₋₅cycloalkyl; and
each R^{N1} independently is H or C₁₋₄alkyl.

Provided herein as Embodiment 59 is the compound or salt of Embodiment 58, wherein at least one of R^{1a}, R^{1b}, and R² is H or D.

Provided herein as Embodiment 60 is the compound or salt of Embodiment 59, wherein each of R^{1a}, R^{1b}, and R² independently is H or D.

Provided herein as Embodiment 61 is the compound or salt of Embodiment 60, wherein each of R^{1a}, R^{1b}, and R² is H.

Provided herein as Embodiment 62 is the compound or salt of Embodiment 60, wherein each of R^{1a}, R^{1b}, and R² is D.

Provided herein as Embodiment 63 is the compound or salt of Embodiment 58 or 59, wherein at least one of R^{1a}, R^{1b}, and R² is halo.

Provided herein as Embodiment 64 is the compound or salt of Embodiment 63, wherein R^{1a} is halo and each of RED and R² is H.

Provided herein as Embodiment 65 is the compound or salt of Embodiment 63 or 64, wherein each halo independently is Br, Cl, or F.

Provided herein as Embodiment 66 is the compound or salt of Embodiment 58 or 59, wherein at least one of R^{1a}, R^{1b}, and R² is C₁₋₄alkyl or C₁₋₄haloalkyl.

Provided herein as Embodiment 67 is the compound or salt of Embodiment 66, wherein at least one of R^{1a}, R^{1b}, and R² is CH₃, CH₂F, CHF₂, or CF₃.

Provided herein as Embodiment 68 is the compound or salt of Embodiment 58 or 59, wherein at least one of R^{1a}, R^{1b}, and R² is C₁₋₂alkylene-OH, C₀₋₂alkylene-C₁₋₃alkoxy, C₀₋₂alkylene-C₁₋₄haloalkoxy, C₀₋₂alkylene-CN, or C₀₋₂alkylene-N(R^{N1})₂.

Provided herein as Embodiment 69 is the compound or salt of Embodiment 68, wherein each R^{N1} independently is H or CH₃.

Provided herein as Embodiment 70 is the compound or salt of Embodiment 69, wherein each R^{N1} is H.

Provided herein as Embodiment 71 is the compound or salt of Embodiment 68 or 69, wherein at least one of R^{1a}, R^{1b}, and R² is CH₂OH, OCH₃, CH₂OCH₃, OCF₃, CH₂OCF₃, CN, CH₂CN, NH₂, N(CH₃)₂, CH₂NH₂, or CH₂N(CH₃)₂.

Provided herein as Embodiment 72 is the compound or salt of Embodiment 58 or 59, wherein at least one of R^{1a}, R^{1b}, and R² is C₁₋₂alkylene-heterocycloalkyl having 3-6 total ring atoms and 1 or 2 heteroatoms selected from N, O, and S.

Provided herein as Embodiment 73 is the compound or salt of Embodiment 72, wherein the heterocycloalkyl is aziridinyl, azetidinyl, pyrrolidinyl, piperidinyl, or morpholinyl.

Provided herein as Embodiment 74 is the compound or salt of Embodiment 72 or 73, wherein at least one of R^{1a}, R^{1b}, and R² is aziridin-1-yl-methyl, azetidin-1-yl-methyl, pyrrolidine-1-yl-methyl, piperidin-1-yl-methyl, or morpholin-1-yl-methyl.

Provided herein as Embodiment 75 is the compound or salt of Embodiment 58 or 59, wherein R^{1b} and R², together with the carbon atoms to which they are attached, form

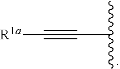

Provided herein as Embodiment 76 is the compound or salt of Embodiment 58, wherein

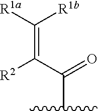

is

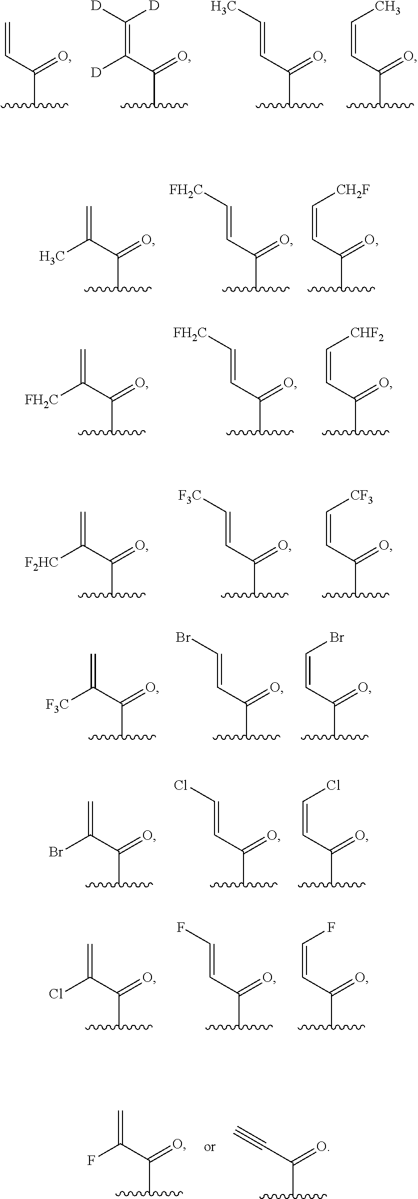

Provided herein as Embodiment 77 is the compound or salt of Embodiment 76, wherein

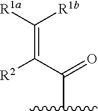

is

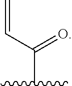 [SMILES: *C(=O)C=C]

Provided herein as Embodiment 78 is the compound or salt of any one of Embodiments 58-77, wherein m is 0.

Provided herein as Embodiment 79 is the compound or salt of any one of Embodiments 58-77, wherein m is 1.

Provided herein as Embodiment 80 is the compound or salt of any one of Embodiments 58-77, wherein m is 2.

Provided herein as Embodiment 81 is the compound or salt of any one of Embodiments 58-77, wherein m is 3.

Provided herein as Embodiment 82 is the compound or salt of any one of Embodiments 58-77, wherein m is 4.

Provided herein as Embodiment 83 is the compound or salt of any one of Embodiments 58-82, wherein

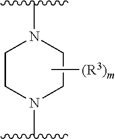

is deuterated.

Provided herein as Embodiment 84 is the compound or salt of Embodiment 83, wherein

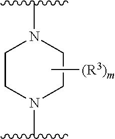

is

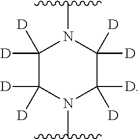

Provided herein as Embodiment 85 is the compound of salt of any one of Embodiments 79-82, wherein at least one R³ is C₁₋₃alkyl or C₁₋₃haloalkyl.

Provided herein as Embodiment 86 is the compound or salt of Embodiment 85, wherein at least one R³ is CH₃, CH₂CH₃, CF₃, CHF₂, or CH₂F.

Provided herein as Embodiment 87 is the compound or salt of Embodiment 86, wherein at least one R³ is CH₃.

Provided herein as Embodiment 88 is the compound or salt of any one of Embodiments 79-82, wherein at least one R³ is

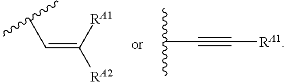

Provided herein as Embodiment 89 is the compound or salt of Embodiment 88, wherein each of R^{A1} and R⁴² independently is H, CH₃, CH₂F, CHF₂, CF₃, CH₂CH₃, CH₂CH—CH₃, CH(CH₃)₂), cyclopropyl, or cyclobutyl.

Provided herein as Embodiment 90 is the compound of Embodiment 88 or 89, wherein at least one R³ is

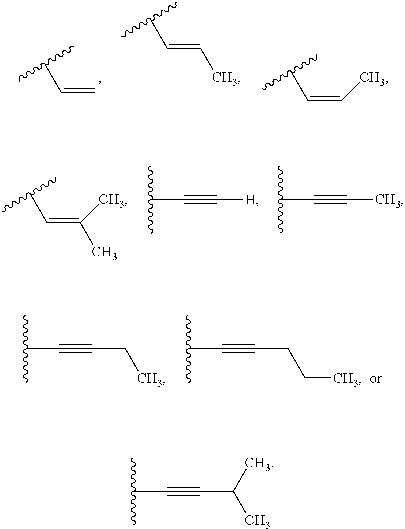

Provided herein as Embodiment 91 is the compound or salt of any one of Embodiments 79-82, wherein at least one R³ is C₀₋₃alkyleneCN.

Provided herein as Embodiment 92 is the compound or salt of Embodiment 91, wherein at least one R³ is CN or CH₂CN.

Provided herein as Embodiment 93 is the compound or salt of any one of Embodiments 79-82, wherein at least one R³ is C₀₋₆alkyleneOH or C₀₋₆alkylene-C₁₋₃alkoxy.

Provided herein as Embodiment 94 is the compound or salt of Embodiment 93, wherein at least one R³ is OH, CH₂OH, CH₂CH₂OH, OCH₃, CH₂OCH₃, or CH—CH₂OCH₃.

Provided herein as Embodiment 95 is the compound or salt of any one of Embodiments 80-82, wherein two geminal R³, together with the atom to which they are attached, form oxo.

Provided herein as Embodiment 96 is the compound or salt of any one of Embodiments 80-82, wherein two geminal R³, together with the atom to which they are attached, form spiro-C₃₋₇cycloalkyl or spiro-heterocycloalkyl having 3-7 total ring atoms and 1 or 2 heteroatoms selected from N, O and S.

Provided herein as Embodiment 97 is the compound or salt of Embodiment 96, wherein two geminal R³, together with the atom to which they are attached, form spiro-cyclopropyl, spiro-cyclobutyl, spiro-oxetanyl, or spiro-tetrahydrofuranyl.

Provided herein as Embodiment 98 is the compound or salt of any one of Embodiments 80-82, wherein two geminal R³, together with the atom to which they are attached, form spiro-C₄-cycloalkenyl or spiro-heterocycloalkenyl having 4-7 total ring atoms and 1 or 2 heteroatoms selected from N, O and S.

Provided herein as Embodiment 99 is the compound or salt of any one of Embodiments 80-82, wherein two vicinal R³, together with the atoms to which they are attached, form fused-C₃₋₇cycloalkyl or fused-heterocycloalkyl having 3-7 total ring atoms and 1 or 2 heteroatoms selected from N, O and S.

Provided herein as Embodiment 100 is the compound or salt of Embodiment 99, wherein two vicinal R³, together with the atoms to which they are attached, form fused-cyclopropyl or fused-cyclobutyl.

Provided herein as Embodiment 101 is the compound or salt of any one of Embodiments 80-82, wherein two vicinal R³, together with the atoms to which they are attached, form fused-C₄-7cycloalkenyl or fused-heterocycloalkenyl having 4-7 total ring atoms and 1 or 2 heteroatoms selected from N, O and S.

Provided herein as Embodiment 102 is the compound or salt of any one of Embodiments 79-82, wherein each R³ independently is CH₃, CH₂CH₃, CH₂F, CHF₂, CF₃, CN, CH₂CN, OH, CH₂OH, CH₂CH₂OH, OCH₃, CH₂OCH₃, or CH₂CH₂OCH₃; two geminal R³, together with the atom to which they are attached, form oxo, spiro-cyclopropyl, spiro-cyclobutyl, spiro-oxetanyl, or spiro-tetrahydrofuranyl; or two vicinal R³, together with the atoms to which they are attached, form fused-cyclopropyl or fused-cyclobutyl.

Provided herein as Embodiment 103 is the compound or salt of any one of Embodiments 58-77, wherein m is 0; or m is 1 and R³ is CH₃, CH₂F, CHF₂, CF₃, CN, CH₂CN, CH₂OH, or CH₂OCH₃; or m is 2 and two geminal R³, together with the atom to which they are attached, form spiro-oxetanyl.

Provided herein as Embodiment 104 is the compound or salt of Embodiment 103, wherein m is 0; or m is 1 and R³ is CH₃.

Provided herein as Embodiment 105 is the compound or salt of any one of Embodiments 58-77, wherein

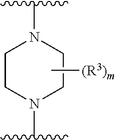

is

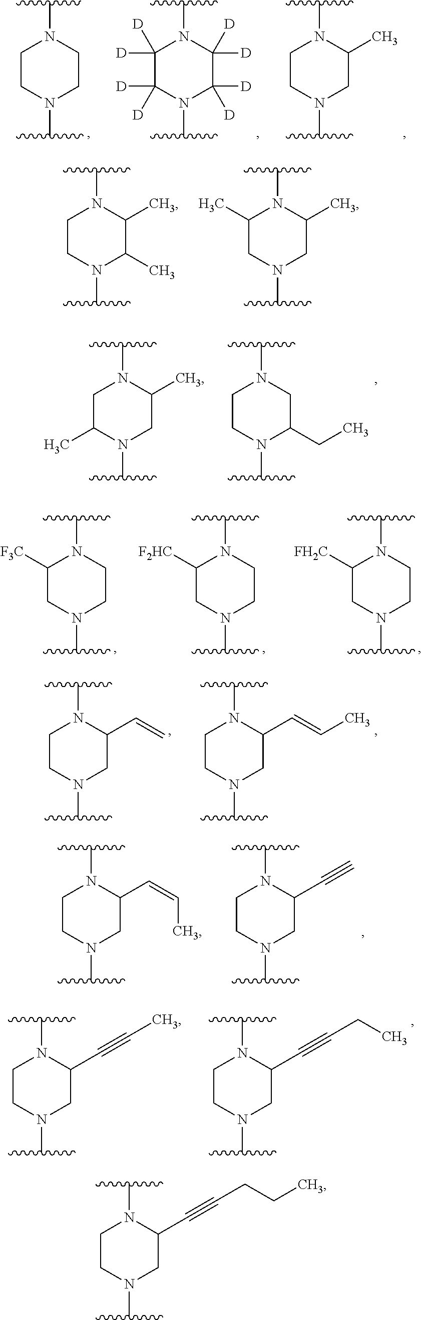

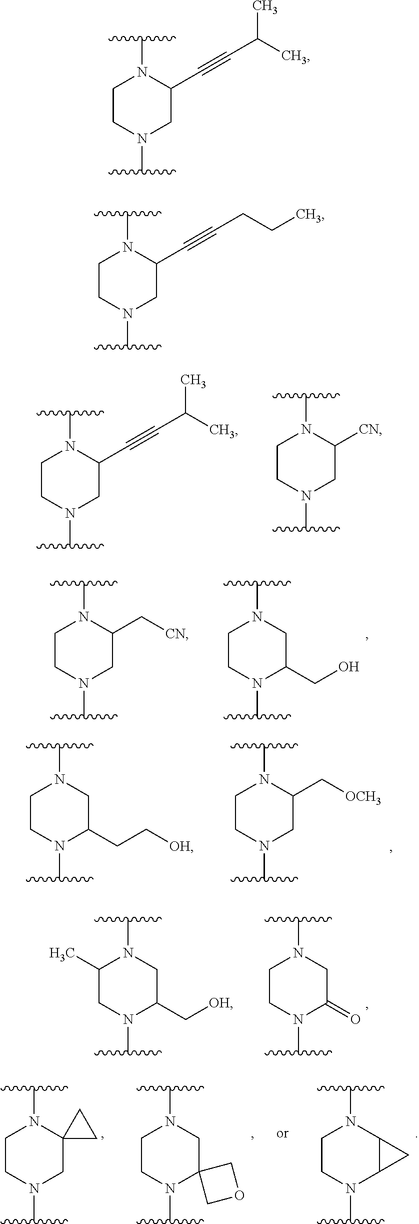

Provided herein as Embodiment 106 is the compound or salt of Embodiment 105, wherein

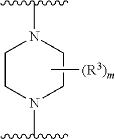

is

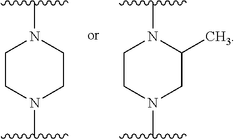 [SMILES: *N1CCN(*)CC1 ; "or" ; *N1CCN(*)C(C)C1]

Provided herein as Embodiment 107 is the compound or salt of any one of Embodiments 58-106, wherein A is N, CH, or C—C₁₋₃alkyl.

Provided herein as Embodiment 108 is the compound or salt of any one of Embodiments 58-107, wherein A is N.

Provided herein as Embodiment 109 is the compound or salt of any one of Embodiments 58-106, wherein A is CH, C-halo, C—CN, C—C₁₋₆alkyl, C—C₁₋₃haloalkyl, C—C₀₋₂alkyleneOH, or C—C₀₋₃alkylene-C₁₋₄alkoxy.

Provided herein as Embodiment 110 is the compound or salt of Embodiment 107 or 109, wherein A is CH.

Provided herein as Embodiment 111 is the compound or salt of Embodiment 107 or 109, wherein A is C—CH₃.

Provided herein as Embodiment 112 is the compound or salt of Embodiment 109, wherein A is C—F, C—Cl, or C—CN.

Provided herein as Embodiment 113 is the compound or salt of Embodiment 109, wherein A is C—CH₂F, C—CHF₂, or C—CF₃.

Provided herein as Embodiment 114 is the compound or salt of Embodiment 109, wherein A is C—C₀₋₃alkyleneOH or C—C₀₋₃alkylene-C₁₋₄alkoxy.

Provided herein as Embodiment 115 is the compound or salt of Embodiment 114, wherein A is C—OH, C—CH₂OH, C—OCH₃, or C—CH₂OCH₃.

Provided herein as Embodiment 116 is the compound or salt of any one of Embodiments 58-115, wherein n is 0.

Provided herein as Embodiment 117 is the compound or salt of any one of Embodiments 58-115, wherein n is 1.

Provided herein as Embodiment 118 is the compound or salt of any one of Embodiments 58-115, wherein n is 2.

Provided herein as Embodiment 119 is the compound or salt of Embodiment 117 or 118, wherein at least one R⁴ is C₁₋₃alkyl or C₁₋₃haloalkyl.

Provided herein as Embodiment 120 is the compound or salt of Embodiment 119, wherein at least one R⁴ is CH₃, CH₂CH., CH₂CH₂CH₃, CH(CH₃)₂, CH₂F, CHF₂, or CF₃.

Provided herein as Embodiment 121 is the compound or salt of Embodiment 120, wherein at least one R⁴ is CH₃.

Provided herein as Embodiment 122 is the compound or salt of Embodiment 117 or 118, wherein at least one R⁴ is C₀₋₃alkyleneCN.

Provided herein as Embodiment 123 is the compound or salt of Embodiment 122, wherein at least one R⁴ is CN or CH₂CN.

Provided herein as Embodiment 124 is the compound or salt of Embodiment 117 or 118, wherein at least one R⁴ is C₁₋₃alkyleneOH or C₁₋₃alkylene-C₁₋₃alkoxy.

Provided herein as Embodiment 125 is the compound or salt of Embodiment 124, wherein at least one R⁴ is CH₂OH, CH₂CH₂OH, CH₂OCH₃, or CH₂CH₂OCH₃.

Provided herein as Embodiment 126 is the compound or salt Embodiment 118, wherein two geminal R⁴, together with the atom to which they are attached, form oxo.

Provided herein as Embodiment 127 is the compound or salt of Embodiment 118, wherein two geminal R⁴, together with the atom to which they are attached, form spiro-C₃.-cycloalkyl or spiro-heterocycloalkyl having 4-7 total ring atoms and 1 or 2 heteroatoms selected from N, O and S.

Provided herein as Embodiment 128 is the compound or salt of Embodiment 127, wherein two geminal R⁴, together with the atom to which they are attached, form spiro-cyclopropyl, spiro-cyclobutyl, or spiro-oxetanyl.

Provided herein as Embodiment 129 is the compound or salt of any one of Embodiments 58-108, wherein

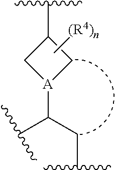

is

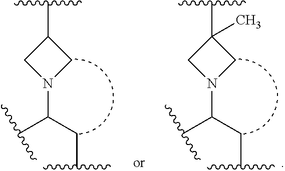

Provided herein as Embodiment 130 is the compound or salt of Embodiment any one of Embodiments 58-129, wherein [symbol] is unsubstituted.

Provided herein as Embodiment 131 is the compound or salt of any one of Embodiments 58-129, wherein [symbol] is substituted with 1-4 substituents.

Provided herein as Embodiment 132 is the compound or salt of Embodiment 131, wherein each substituent independently is C₁₋₃alkyl, C₁₋₃haloalkyl, C₂₋₆alkenyl, halo, CN, C₀₋₂alkyleneOH, C₀₋₃alkylene-C₁₋₃alkoxy, C₃₋₅cycloalkyl, C₄₋₅cycloalkenyl, heterocycloalkyl having 4 or 5 total ring atoms and 1-3 heteroatoms selected from N, O, and S, heterocycloalkenyl having 4 or 5 total ring atoms and 1-3 heteroatoms selected from N, O, and S, phenyl; or two geminal substituents, together with the atom to which they are attached, form oxo, ═CH₂, spiro-C₃₋₅cycloalkyl, spiro-C₄₋₅cycloalkenyl, spiro-heterocycloalkyl having 3-5 total ring atoms and 1 or 2 heteroatoms selected from N, O and S, or spiro-heterocycloalkenyl having 4 or 5 total ring atoms and 1 or 2 heteroatoms selected from N, O and S; or two vicinal substituents, together with the atoms to which they are attached, form fused-C₃₋₅cycloalkyl, fused-C₄₋₅cycloalkenyl, fused-heterocycloalkyl having 3-5 total ring atoms and 1 or 2 heteroatoms selected from N, O and S or fused-heterocycloalkenyl having 4 or 5 total ring atoms and 1 or 2 heteroatoms selected from N, O and S.

Provided herein as Embodiment 133 is the compound or salt of Embodiment 132, wherein each substituent independently is C₁₋₃alkyl, C₁₋₃haloalkyl, C₂₋₆alkenyl, halo, C₀₋₃alkyleneOH, C₀₋₃alkylene-C₁₋₃alkoxy; or two geminal substituents, together with the atom to which they are attached, form oxo or =CH₂; or two vicinal substituents, together with the atoms to which they are attached, form fused-C₃₋₅cycloalkyl or fused-heterocycloalkyl having 4 or 5 total ring atoms and 1 or 2 heteroatoms selected from N, O and S.

Provided herein as Embodiment 134 is the compound or salt of Embodiment 133, wherein [symbol] is substituted with CH₃, =CH₂, oxo, Cl, F, OH, OCH₃

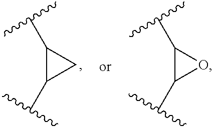 [SMILES: *C1CC1* ; *C1OC1*]

or a combination of the foregoing.

Provided herein as Embodiment 135 is the compound or salt of any one of Embodiments 58-134, wherein [symbol] is C₂₋₆alkylene.

Provided herein as Embodiment 136 is the compound or salt of Embodiment 135, wherein [symbol] is C₂alkylene.

Provided herein as Embodiment 137 is the compound or salt of any one of Embodiments 58-129, wherein [symbol] is

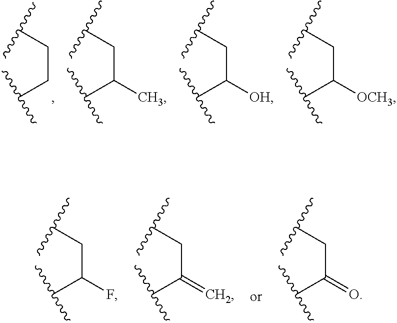

Provided herein as Embodiment 138 is the compound or salt of Embodiment 135, wherein [symbol] is C₃alkylene.

Provided herein as Embodiment 139 is the compound or salt of any one of Embodiments 58-129, wherein [symbol] is

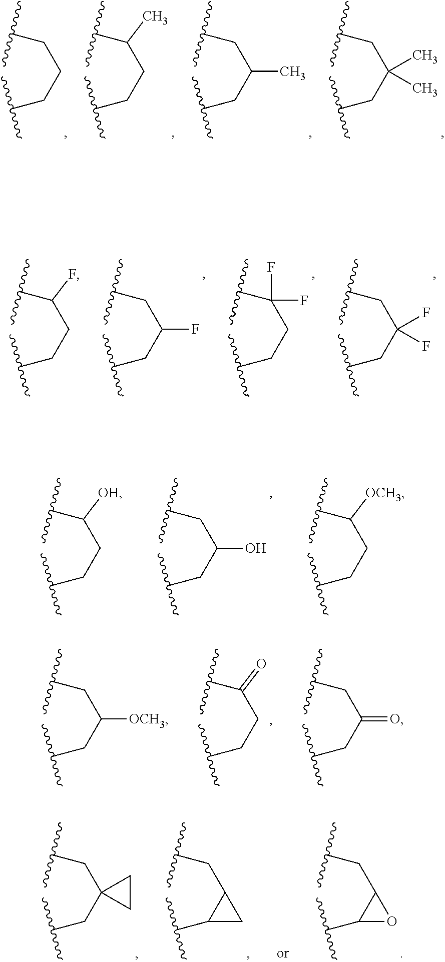

Provided herein as Embodiment 140 is the compound or salt of Embodiment 135, wherein [symbol] is C₀₋₃alkylene.

Provided herein as Embodiment 141 is the compound or salt of Embodiment 140, wherein [symbol] is

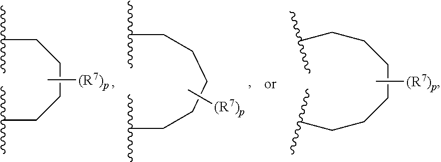

wherein p is 0, 1, 2, or 3, and each R⁷ independently is CH₃, Cl, F, OH, or OCH₃; or two geminal R⁷, together with the atom to which they are attached form oxo or =CH₂; or two vicinal R⁷, together with the atoms to which they are attached form

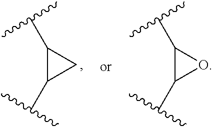 [SMILES: *C1CC1* ; *C1OC1*]

Provided herein as Embodiment 142 is the compound or salt of any one of Embodiments 58-134, wherein [symbol] is C₂₋₆alkenylene.

Provided herein as Embodiment 143 is the compound or salt of Embodiment 142, wherein [symbol] is C₃alkenylene

Provided herein as Embodiment 144 is the compound or salt of any one of Embodiments 58-129, wherein [symbol] is

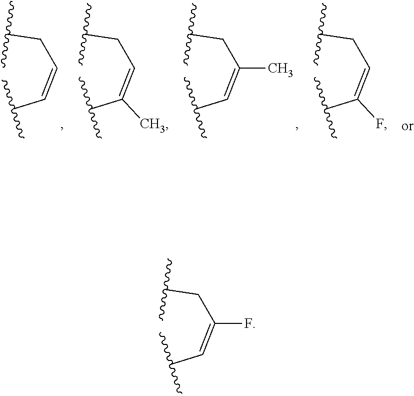

Provided herein as Embodiment 145 is the compound or salt of Embodiment 141, wherein [symbol] is C₂₋₆alkenylene.

Provided herein as Embodiment 146 is the compound or salt of Embodiment 145, wherein [symbol] is

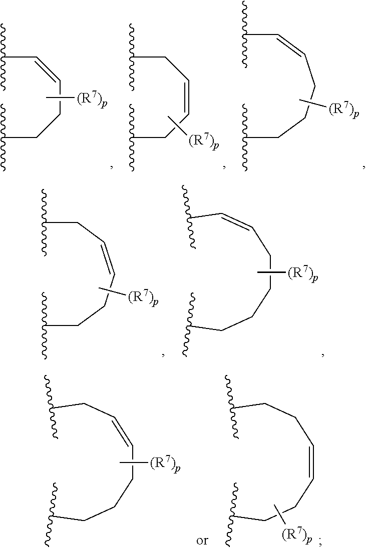

p is 0, 1, 2, or 3, and each R⁷ independently is CH₃, Cl, F, OH, OCH₃; or two geminal R⁷, together with the atom to which they are attached form oxo or ═CH₂; or two vicinal R⁷, together with the atoms to which they are attached form

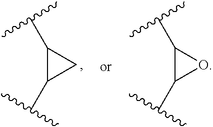 [SMILES: *C1CC1* ; *C1OC1*]

Provided herein as Embodiment 147 is the compound or salt of any one of Embodiments 58-134, wherein [symbol] is heteroalkylene having 2-6 total atoms and 1-3 heteroatoms selected from N, O, and S.

Provided herein as Embodiment 148 is the compound or salt of Embodiment 147, wherein the heteroalkylene has 2-4 total atoms and 1 or 2 heteroatoms selected from N, O, and S.

Provided herein as Embodiment 149 is the compound or salt of any one of Embodiments 58-129, wherein [symbol] is

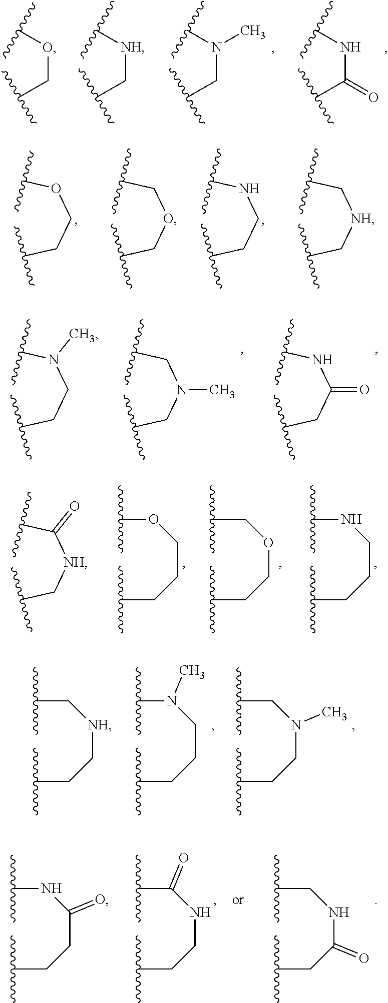

Provided herein as Embodiment 150 is the compound or salt of Embodiment 149, wherein [symbol] is

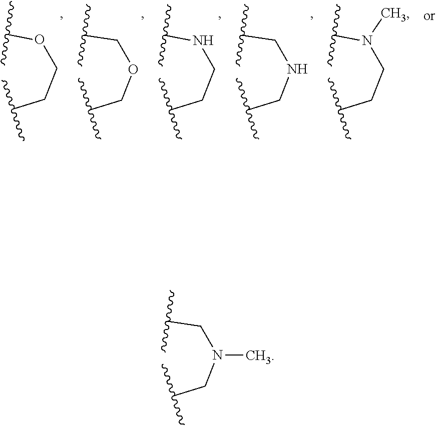

Provided herein as Embodiment 151 is the compound or salt of any one of Embodiments 58-134, wherein [symbol] is heteroalkenylene having 3-6 total atoms and 1 or 2 heteroatoms selected from N, O, and S.

Provided herein as Embodiment 152 is the compound or salt of any one of Embodiments 58-129, wherein [symbol] is

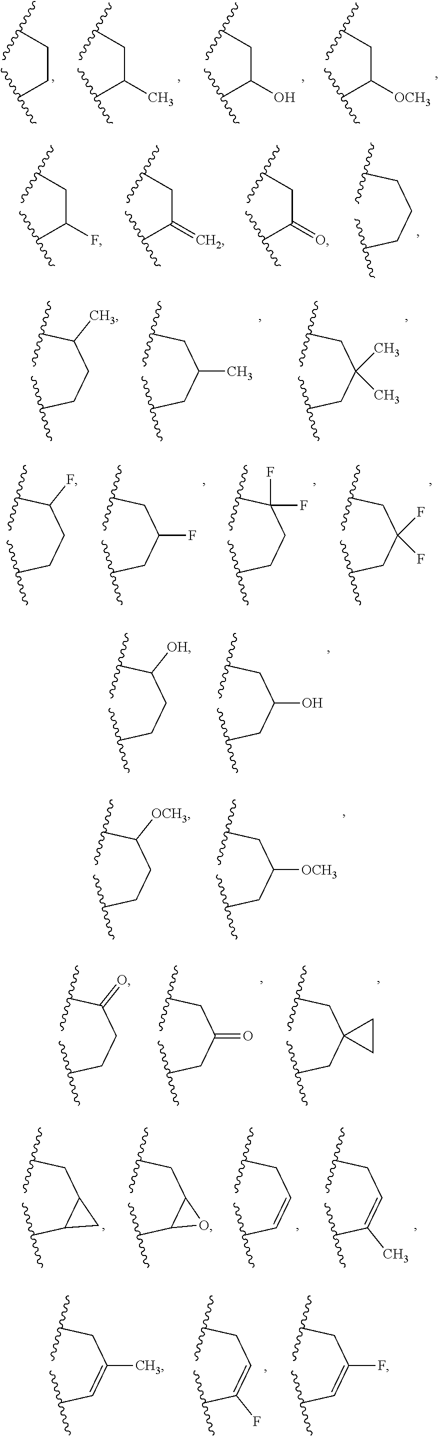

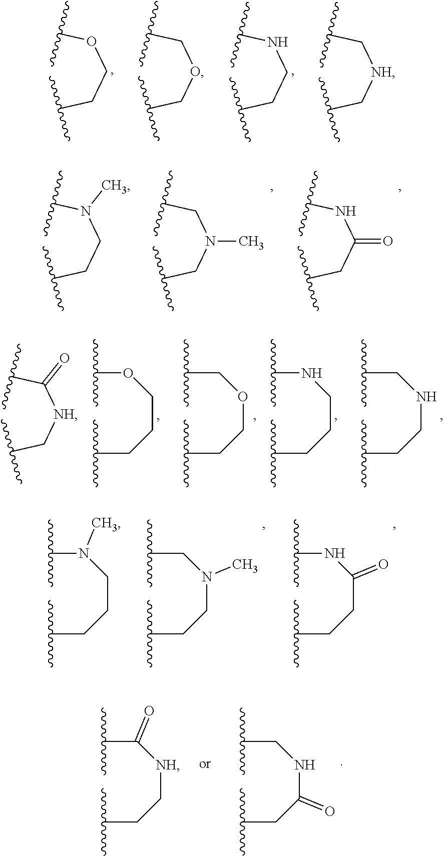

Provided herein as Embodiment 153 is the compound or salt of Embodiment 152, wherein [symbol] is

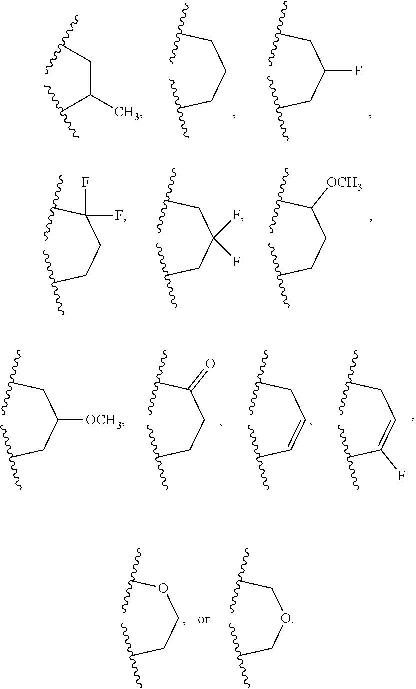

Provided herein as Embodiment 154 is the compound or salt of Embodiment 153, wherein [symbol] is

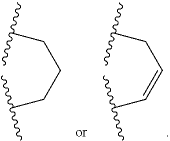 [SMILES: *CCC* ; */C=C\C*]

Provided herein as Embodiment 155 is the compound or salt of any one of Embodiments 58-154, wherein W¹ is N.

Provided herein as Embodiment 156 is the compound or salt of any one of Embodiments 58-154, wherein W¹ is CH.

Provided herein as Embodiment 157 is the compound or salt of any one of Embodiments 58-154, wherein W¹ is C—F, C—Cl, or C—CN.

Provided herein as Embodiment 158 is the compound or salt of any one of Embodiments 58-154, wherein W¹ is C—C₁₋₃alkyl or C—C₁₋₃haloalkyl.

Provided herein as Embodiment 159 is the compound or salt of Embodiment 158, wherein W¹ is C—CH₃, C—CH₂CH₃, C—CH₂F, C—CHF₂, or C—CF₃.

Provided herein as Embodiment 160 is the compound or salt of any one of Embodiments 58-154, wherein W¹ is C—C₂₋₃alkenyl or C—C₂₋₃alkynyl, and each of the alkenyl and alkynyl is unsubstituted or substituted with 1 or more substituents.

Provided herein as Embodiment 161 is the compound or salt of Embodiment 160, wherein each of the C—C₂₋₃alkenyl and C—C₂₋₃alkynyl is unsubstituted.

Provided herein as Embodiment 162 is the compound or salt of Embodiment 160, wherein each of the C—C₂₋₃alkenyl and C—C₂₋₃alkynyl is substituted with 1-3 substituents, and each substituent independently is halo, C₁₋₃haloalkyl, C₀₋₆alkyleneOH, or C₀₋₃alkyleneC₁₋₄alkoxy

Provided herein as Embodiment 163 is the compound or salt of Embodiment 160, wherein W¹ is C—CH═CH₂, C—C(OH)—CH₃, C—CH—CH(OH), or C—CCH.

Provided herein as Embodiment 164 is the compound or salt of any one of Embodiments 58-154, wherein W¹ is C—C₀₋₂alkyleneOH or C—C₀₋₃alkylene-C₁₋₄alkoxy.

Provided herein as Embodiment 165 is the compound or salt of Embodiment 164, wherein W¹ is C—OH, C—CH₂OH, C—OCH₃, or C₂CH₂OCH₃.

Provided herein as Embodiment 166 is the compound or salt of any one of Embodiments 58-165, wherein W² is N.

Provided herein as Embodiment 167 is the compound or salt of any one of Embodiments 58-165, wherein W² is CH.

Provided herein as Embodiment 168 is the compound or salt of any one of Embodiments 58-165, wherein W² is C—F, C—C₁, or C—CN.

Provided herein as Embodiment 169 is the compound or salt of any one of Embodiments 58-165, wherein W² is C—C₁₋₃alkyl or C—C₁₋₃haloalkyl.

Provided herein as Embodiment 170 is the compound or salt of Embodiment 169, wherein W² is C—CH₃, C—CH₂CH₃, C—CH₂F, C—CHF₂, or C—CF₃.

Provided herein as Embodiment 171 is the compound or salt of any one of Embodiments 58-165, wherein W² is C—C₂₋₃alkenyl or C—C₂₋₃alkynyl, and each of the alkenyl and alkynyl is unsubstituted or substituted with 1 or more substituents.

Provided herein as Embodiment 172 is the compound or salt of Embodiment 171, wherein each of the C—C₂₋₃alkenyl and C—C₂₋₃alkynyl is unsubstituted.

Provided herein as Embodiment 173 is the compound or salt of Embodiment 171, wherein each of the C—C₂₋₃alkenyl and C—C₂₋₃alkynyl is substituted with 1-3 substituents, and each substituent independently is halo, C₁₋₃haloalkyl, C₀₋₆alkyleneOH, or C₁₋₃alkyleneC₁₋₄alkoxy.

Provided herein as Embodiment 174 is the compound or salt of Embodiment 171, wherein W² is C—CH═CH₂, C—C(OH)=CH₂, C—CH═CH(OH), or C—CCH.

Provided herein as Embodiment 175 is the compound or salt of any one of Embodiments 58-165, wherein W² is C—C₀₋₂alkyleneOH or C—C₀₋₃alkylene-C₁₋₄alkoxy.

Provided herein as Embodiment 176 is the compound or salt of Embodiment 175, wherein W² is C—OH, C—CH₂OH, C—OCH₃, or C₂CH₂OCH₃.

Provided herein as Embodiment 177 is the compound or salt of any one of Embodiments 58-154, wherein each of W¹ and W² independently is N, CH, or C₂CH₃.

Provided herein as Embodiment 178 is the compound or salt of Embodiment 177, wherein W¹ is CH and W² is N, CH, or C₂CH₃.

Provided herein as Embodiment 179 is the compound or salt of Embodiment 177, wherein W² is N and Whis N, CH, or C—CH₃.

Provided herein as Embodiment 180 is the compound or salt of Embodiment 177, wherein W¹ is CH and W² is N.

Provided herein as Embodiment 181 is the compound or salt of Embodiment 180, wherein

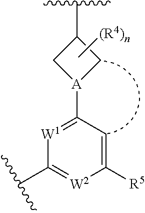

is

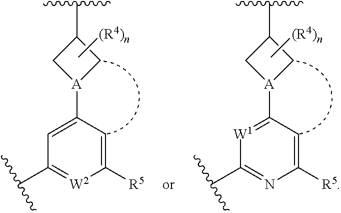

Provided herein as Embodiment 182 is the compound or salt of Embodiment 180, wherein

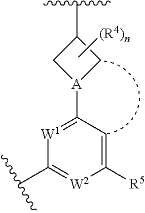

is

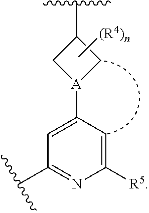

Provided herein as Embodiment 183 is the compound or salt of any one of Embodiments 58-182, wherein R⁵ is Br, Cl, or F.

Provided herein as Embodiment 184 is the compound or salt of any one of Embodiments 58-182, wherein R⁵ is C₁₋₃haloalkyl.

Provided herein as Embodiment 185 is the compound or salt of Embodiment 184, wherein R³ is CF₃, CF₂H, CFH₂, or CF₂CH₃.

Provided herein as Embodiment 186 is the compound or salt of Embodiment 185 wherein R⁵ is CF₃ or CF₂H.

Provided herein as Embodiment 187 is the compound or salt of any one of Embodiments 58-182, wherein R⁵ is Ch-3alkoxy or C₁₋₃thioalkyl.

Provided herein as Embodiment 188 is the compound or salt of Embodiment 187, wherein R⁵ is OCH₃, or SCH₃.

Provided herein as Embodiment 189 is the compound or salt of any one of Embodiments 58-182, wherein R⁵ is C₁₋₆alkyl, C₂₋₄alkenyl, or C₂₋₄alkynyl, and each of the foregoing independently is unsubstituted or substituted with 1-3 substituents.

Provided herein as Embodiment 190 is the compound or salt of Embodiment 189, wherein the alkyl is CH₃, CH₂CH₃, CH₂CH₂CH₃, or CH(CH₃)₂; the alkenyl is CH—CH₂ or CH═CHCH₃; and the alkynyl is

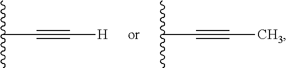

wherein each of the foregoing is unsubstituted or the alkynyl is substituted with 1-3 substituents.

Provided herein as Embodiment 191 is the compound of salt of Embodiment 189 or 190, wherein R⁵ is unsubstituted.

Provided herein as Embodiment 192 is the compound or salt of Embodiment 189 or 190, wherein R⁵ is substituted with 1-3 substituents, and each substituent independently is C₁₋₃haloalkyl, C₀₋₆alkylene-OH, C₀₋₆alkylene-C₁₋₃alkoxy, C₁₋₇cycloalkyl, C₅₋₇cycloalkenyl, heterocycloalkyl having 3-7 total ring atoms and 1-3 heteroatoms selected from N, O, and S, heterocycloalkenyl having 4-7 total ring atoms and 1-3 heteroatoms selected from N, O, and S, or phenyl.

Provided herein as Embodiment 193 is the compound or salt of Embodiment 192 wherein each substituent independently is CH₃, CF₃, CF₂H, CFH₂, OH, OCH₃, OCF₃, CH₂OH, CH₂OCH₃, cyclopropyl, cyclobutyl, or phenyl.

Provided herein as Embodiment 194 is the compound or salt of Embodiment 189, wherein R⁵ is CH₃, CH₂CH₃, CH₂CH₂CH₃, CH(CH₃)₂,

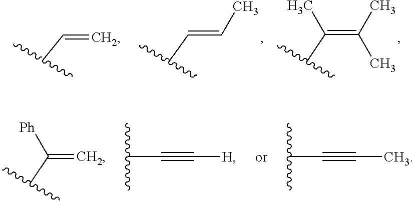

Provided herein as Embodiment 195 is the compound or salt of any one of Embodiments 58-182, wherein R⁵ is C₃-cycloalkyl, C₅₋₇cycloalkenyl, heterocycloalkyl having 3-7 total ring atoms and 1-3 heteroatoms selected from N, O, and S, or heterocycloalkenyl having 5-7 total ring atoms and 1-3 heteroatoms selected from N, O, and S, wherein each of the foregoing is unsubstituted or substituted with 1-3 substituents.

Provided herein as Embodiment 196 is the compound or salt of Embodiment 195, wherein R⁵ is unsubstituted.

Provided herein as Embodiment 197 is the compound or salt of Embodiment 195, wherein R⁵ is substituted with 1-3 substituents, and each substituent independently is halo, C₁₋₃alkyl, C₁₋₃haloalkyl, C₀₋₃alkylene (OH), or C₀₋₃alkylene-C₁₋₃alkoxy.

Provided herein as Embodiment 198 is the compound or salt of any one of Embodiments 58-182, wherein R⁵ is CH₃, CF₃, CF₂H, CFH₂, CH₂CH₃, CH₂CH₂CH₃, CH(CH₃)₂,

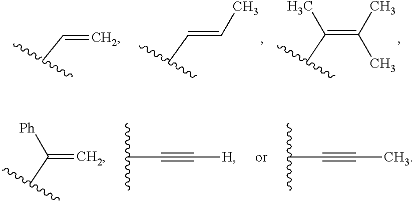

Provided herein as Embodiment 199 is the compound or salt of any one of Embodiments 58-154, wherein W¹ is CH, W² is N, and R⁵ is CF₃, CF H, or CFH₂.

Provided herein as Embodiment 200 is the compound or salt of any one of Embodiments 58-106, wherein

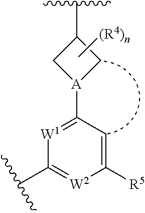

is

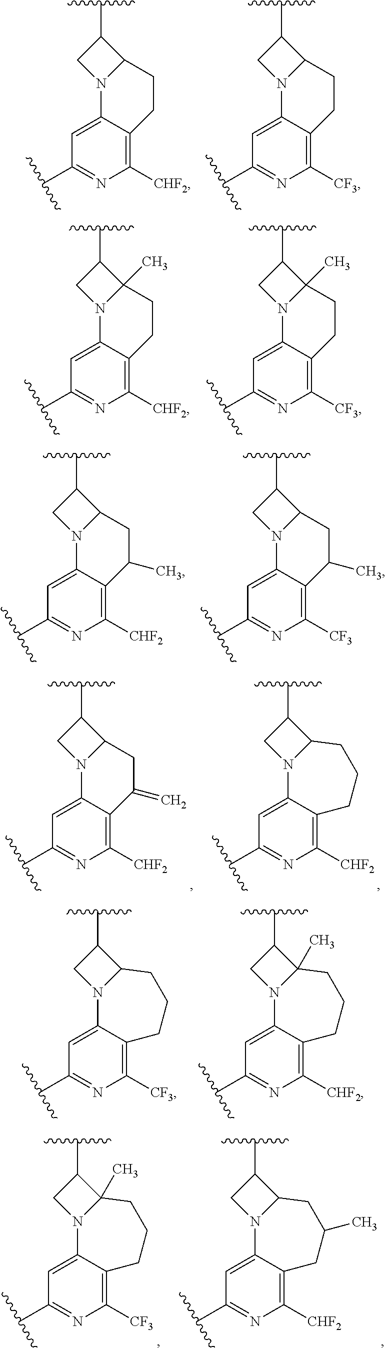

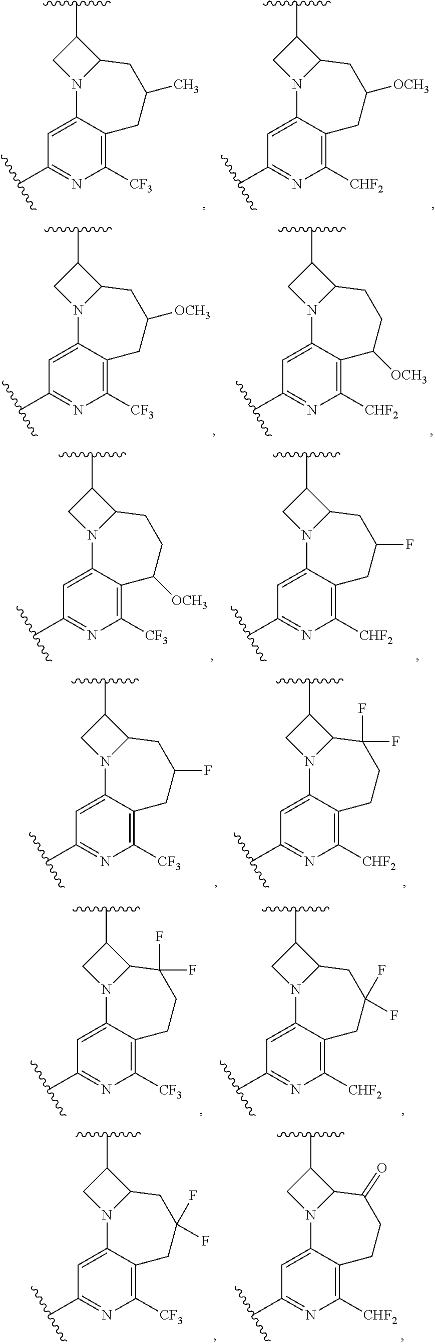

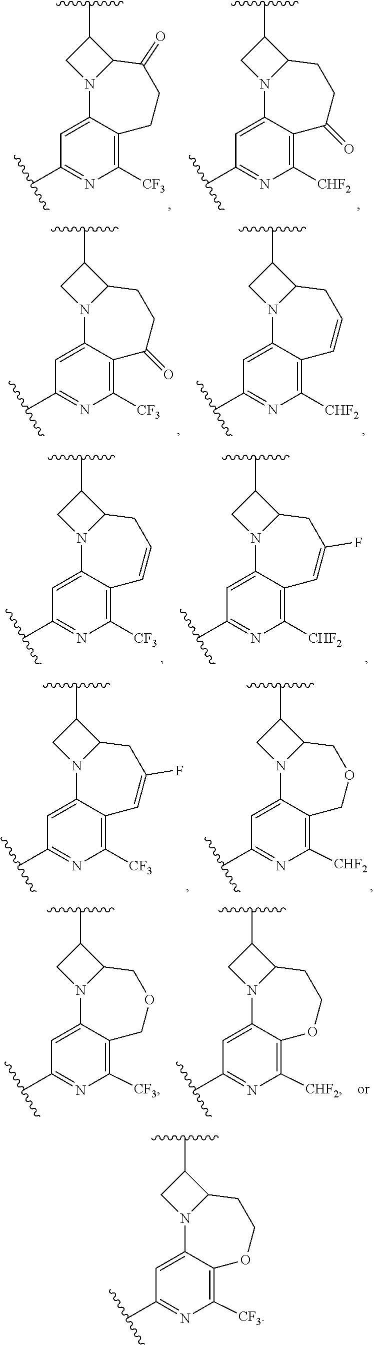

Provided herein as Embodiment 201 is the compound or salt of Embodiment 200, wherein

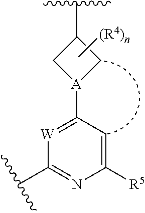

is

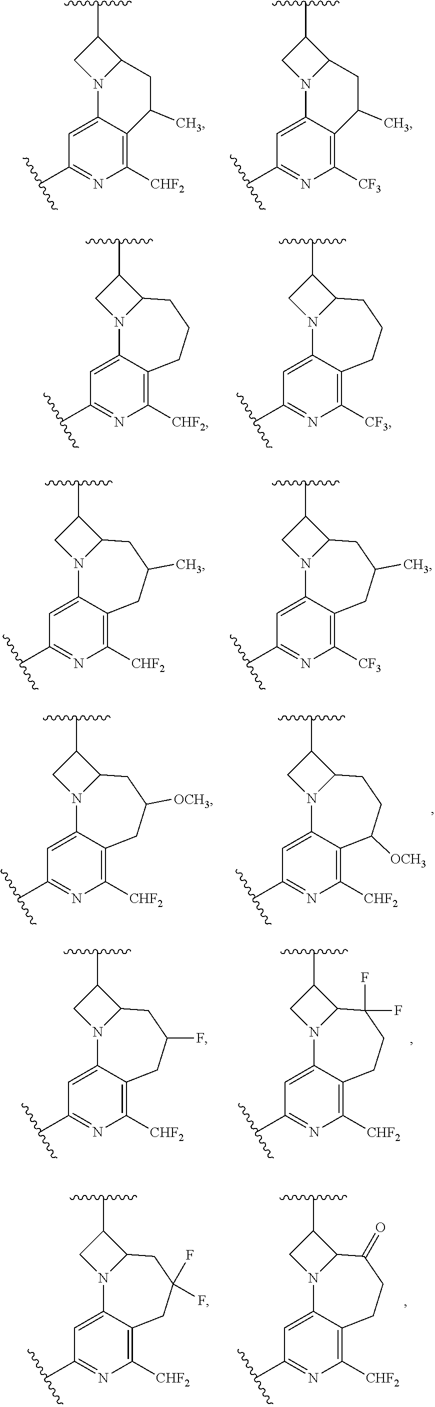

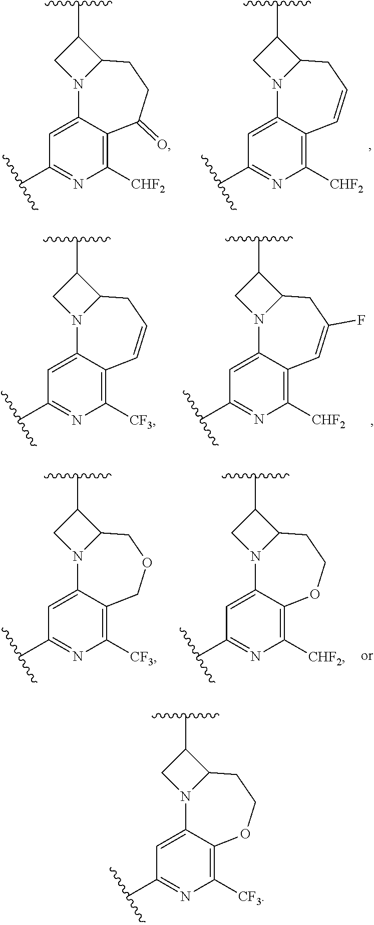

Provided herein as Embodiment 202 is the compound of Embodiment 201, wherein

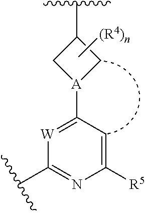

is

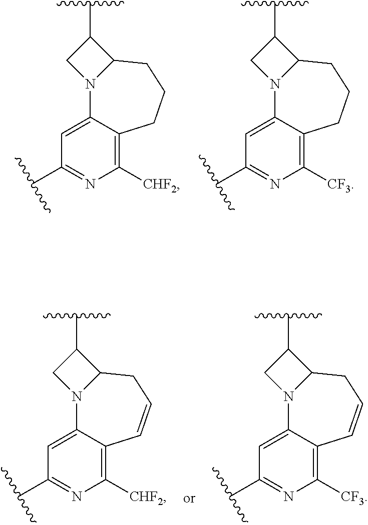 [SMILES: *c1cc2c(c(C(F)F)n1)CCCC1C(*)CN21 ; *c1cc2c(c(C(F)(F)F)n1)CCCC1C(*)CN21 ; *c1cc2c(c(C(F)F)n1)C=CCC1C(*)CN21 ; *c1cc2c(c(C(F)(F)F)n1)C=CCC1C(*)CN21]

Provided herein as Embodiment 203 is the compound or salt of any one of Embodiments 58-202, wherein: X is

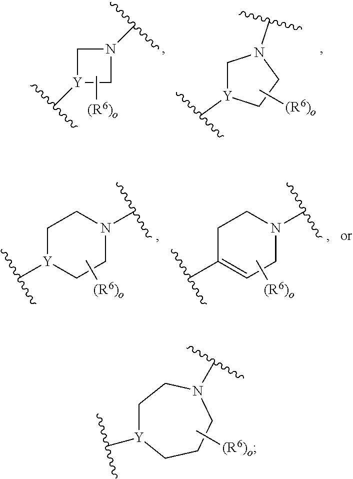

Y is N, C—H, C-halo, C—CN, C—C₁₋₃alkyl, C—C₁₋₃haloalkyl, C—C₀₋₂alkyleneOH, or C—C₀₋₃alkylene-C₁₋₄alkoxy; o is 0, 1, 2, 3, or 4; and each R⁶ independently is halo, CN, C₁₋₃alkyl, C₂₋₃alkenyl, C₁₋₃haloalkyl, C₀₋₂alkylene-OH, C₀₋₂alkylene-C₁₋₃alkoxy, deuterated C₀₋₃alkylene-C₁₋₃alkoxy, or C₁₋₄alkylene-N(R^{N1})₂; or two geminal R⁶, together with the atom to which they are attached, form oxo, ═CH₂, spiro-C₃₋₇cycloalkyl, spiro-C₄₋₇cycloalkenyl, spiro-heterocycloalkyl having 4-7 total ring atoms and 1 or 2 heteroatoms selected from N, O and S, or spiro-heterocycloalkenyl having 4-7 total ring atoms and 1 or 2 heteroatoms selected from N, O and S; or two vicinal R⁶, together with the atoms to which they are attached, form fused-C₃₋₇cycloalkyl, fused-C₄₋₇cycloalkenyl, fused-heterocycloalkyl having 4-7 total ring atoms and 1-3 heteroatoms selected from N, O, and S, or fused-heterocycloalkenyl having 4-7 total ring atoms and 1 or 2 heteroatoms selected from N, O, and S; or two non-neighboring R⁶ join together to form a C₁₋₃alkylene bridge, a C₂₋₃alkenylene bridge, a C₁₋₃ether bridge, or a C₁₋₃thioether bridge; or Y and a vicinal R⁶, together with the atoms to which they are attached, form fused-C₃₋₇cycloalkyl, fused-C₄₋₇cycloalkenyl, fused-heterocycloalkyl having 3-7 total ring atoms and 1-3 heteroatoms selected from N, O, and S, or fused-heterocycloalkenyl having 4-7 total ring atoms and 1 or 2 heteroatoms selected from N, O, and S; wherein the cycloalkyl, cycloalkenyl, heterocycloalkyl or heterocycloalkenyl of any of the foregoing is unsubstituted or substituted with 1 or more substituents; and each R^{N1} independently is H or C₁₋₄alkyl.

Provided herein as Embodiment 204 is the compound or salt of Embodiment 203, wherein o is 0.

Provided herein as Embodiment 205 is the compound or salt of Embodiment 203, wherein o is 1.

Provided herein as Embodiment 206 is the compound or salt of Embodiment 203, wherein o is 2.

Provided herein as Embodiment 207 is the compound or salt of Embodiment 203, wherein o is 3.

Provided herein as Embodiment 208 is the compound or salt of Embodiment 203, wherein o is 4.

Provided herein as Embodiment 209 is the compound or salt of any one of Embodiments 205-208, wherein at least one R⁶ is Br, Cl, F, or CN.

Provided herein as Embodiment 210 is the compound or salt of Embodiment 209, wherein at least one R⁵ is F.

Provided herein as Embodiment 211 is the compound or salt of any one of Embodiments 205-208, wherein at least one R⁶ is C₁₋₃alkyl or C₁₋₃haloalkyl.

Provided herein as Embodiment 212 is the compound or salt of Embodiment 211, wherein at least one R⁶ is CH₃, CH₂F, CHF₂, or CF₃.

Provided herein as Embodiment 213 is the compound or salt of any one of Embodiments 205-208, wherein at least one R⁶ is C₀₋₆alkylene-OH, C₀₋₆alkylene-C₁₋₃alkoxy, deuterated C₀₋₃alkylene-C₁₋₃alkoxy, or C₁₋₄alkylene-N(R^{N1})₂, and each R^{N1} independently is H or CH₃.

Provided herein as Embodiment 214 is the compound or salt of Embodiment 213, wherein at least one R⁶ is OH, CH₂OH, OCH₃, OCD₃, CH₂OCH₃, or CH₂N(CH₃)₂.

Provided herein as Embodiment 215 is the compound or salt of any one of Embodiments 206-208, wherein two geminal R⁶, together with the atom to which they are attached, form oxo or ═CH₂.

Provided herein as Embodiment 216 is the compound or salt of any one of Embodiments 2206-208, wherein two geminal R⁶, together with the atom to which they are attached, form spiro-C₃₋₇cycloalkyl, spiro-C₄₋₇cycloalkenyl, spiro-heterocycloalkyl having 4-7 total ring atoms and 1 or 2 heteroatoms selected from N, O and S, or spiro-heterocycloalkenyl having 4-7 total ring atoms and 1 or 2 heteroatoms selected from N, O and S, wherein any of the foregoing is unsubstituted or substituted with 1 or more substituents.

Provided herein as Embodiment 217 is the compound or salt of Embodiment 216, wherein two geminal R⁶, together with the atom to which they are attached, form spiro-C₃₋₇cycloalkyl or spiro-heterocycloalkyl having 4-7 total ring atoms and 1 or 2 heteroatoms selected from N, O and S, wherein any of the foregoing is unsubstituted or substituted with 1 or more substituents.

Provided herein as Embodiment 218 is the compound or salt of Embodiment 217, wherein two geminal R⁶, together with the atom to which they are attached, form spiro-cyclopropyl, spiro-cyclobutyl, spiro-oxetanyl, or spiro-tetrahydrofuranyl, wherein any of the foregoing is unsubstituted or substituted with 1 or more substituents.

Provided herein as Embodiment 219 is the compound or salt of any one of Embodiments 206-208, wherein two vicinal R⁶, together with the atoms to which they are attached, form fused-C₃₋₇cycloalkyl, fused-C₃₋₇cycloalkenyl, fused-heterocycloalkyl having 4-7 total ring atoms and 1-3 heteroatoms selected from N, O, and S, or fused-heterocycloalkenyl having 4-7 total ring atoms and 1 or 2 heteroatoms selected from N, O, and S, or Y and a vicinal R⁶, together with the atoms to which they are attached, form fused-C₃₋₇cycloalkyl, fused-C₃₋₇cycloalkenyl, fused-heterocycloalkyl having 3-7 total ring atoms and 1-3 heteroatoms selected from N, O, and S, or fused-heterocycloalkenyl having 4-7 total ring atoms and 1 or 2 heteroatoms selected from N, O, and S; wherein the cycloalkyl, cycloalkenyl, heterocycloalkyl, or heterocycloalkenyl of any of the foregoing is unsubstituted or substituted with 1 or more substituents.

Provided herein as Embodiment 220 is the compound or salt of Embodiment 219, wherein two vicinal R⁶, together with the atoms to which they are attached, form fused-C₃₋₇cycloalkyl, or Y and a vicinal R⁶, together with the atoms to which they are attached, form fused-C₃-cycloalkyl, wherein the cycloalkyl of any of the foregoing is unsubstituted or substituted with 1 or more substituents.

Provided herein as Embodiment 221 is the compound or salt of Embodiment 219 or 220, wherein the fused-C₃₋₇cycloalkyl is fused-cyclopropyl, fused-cyclobutyl, or fused-cyclopentyl, and any of the foregoing is unsubstituted or substituted with 1 or more substituents.

Provided herein as Embodiment 222 is the compound or salt of any one of Embodiments 216-221, wherein the cycloalkyl, cycloalkenyl, heterocycloalkyl, or heterocycloalkenyl is unsubstituted.

Provided herein as Embodiment 223 is the compound or salt of any one of Embodiments 216-221, wherein the cycloalkyl, cycloalkenyl, heterocycloalkyl, or heterocycloalkenyl is substituted with 1 or more substituents.

Provided herein as Embodiment 224 is the compound or salt of any one of Embodiments 216-221, wherein the cycloalkyl, cycloalkenyl, heterocycloalkyl, or heterocycloalkenyl is substituted with 1 or 2 substituents.

Provided herein as Embodiment 225 is the compound or salt of Embodiment 223 or 224, wherein each substituent independently is halo, C₁₋₃alkyl, C₁₋₃haloalkyl, C₀₋₃alkyleneOH, C₀₋₂alkyleneC₁₋₃alkoxy, or C₀₋₃alkyleneCN.

Provided herein as Embodiment 226 is the compound or salt of Embodiment 225, wherein each substituent independently is F, Cl, OH, OCH₃, OCH—CH₃, or CN.

Provided herein as Embodiment 227 is the compound or salt of any one of Embodiments 206-208, wherein two non-neighboring R⁶ join together to form a C₁₋₃alkylene bridge, a C₂₋₃alkenylene bridge, a C₁₋₃ether bridge, or a C₁₋₃thioether bridge.

Provided herein as Embodiment 228 is the compound or salt of Embodiment 227, wherein two non-neighboring R⁶ join together to form —CH₂—, —CH₂CH₂—, —CH₂CH₂CH₂—, —CH₂—CH—CH— or —CH₂OCH₂—.

Provided herein as Embodiment 229 is the compound or salt of any one of Embodiments 203-228, wherein Y is N.

Provided herein as Embodiment 230 is the compound or salt of any one of Embodiments 203-228, wherein Y is CH.

Provided herein as Embodiment 231 is the compound or salt of any one of Embodiments 203-228, wherein Y is C-halo, C—CN, C—C₁₋₃alkyl, C—C₁₋₄haloalkyl, C—C₀₋₃alkyleneOH, or C—C₀₋₃alkylene-C₁₋₃alkoxy.

Provided herein as Embodiment 232 is the compound or salt of Embodiment 231, wherein Y is C—F, C—Cl, C₂CH₃, C—CH₂CH₃, C—CH₂F, C—CHF₂, C—CF₃, C—OH, C—CH₂OH, C—OCH₃, or C—CH₂OCH₃.

Provided herein as Embodiment 233 is the compound or salt of any one of Embodiments 203-232, wherein X is

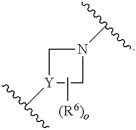

Provided herein as Embodiment 234 is the compound or salt of Embodiment 203, wherein X is

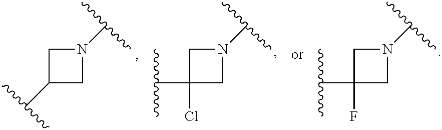 [SMILES: *C1CN(*)C1 ; *N1CC(*)(Cl)C1 ; *N1CC(*)(F)C1]

Provided herein as Embodiment 235 is the compound or salt of Embodiment 234, wherein X is

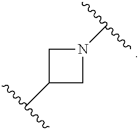 [SMILES: *C1CN(*)C1]

Provided herein as Embodiment 236 is the compound or salt of any one of Embodiments 203-232, wherein X is

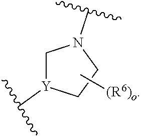

Provided herein as Embodiment 237 is the compound or salt of Embodiment 203, wherein X is

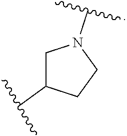 [SMILES: *C1CCN(*)C1]

Provided herein as Embodiment 238 is the compound or salt of any one of Embodiments 203-232, wherein X is

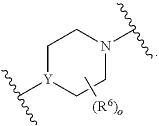

Provided herein as Embodiment 239 is the compound or salt of Embodiment 203, wherein X is

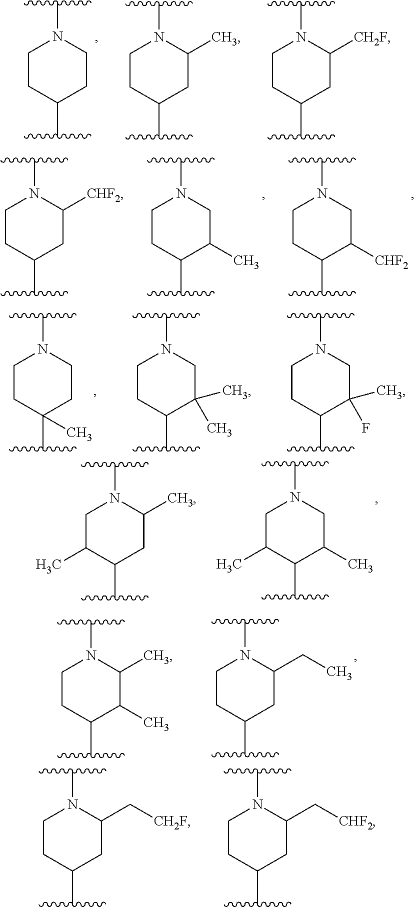

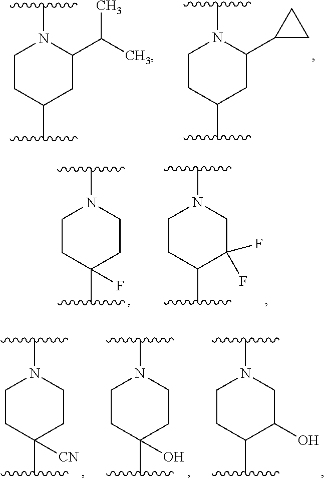

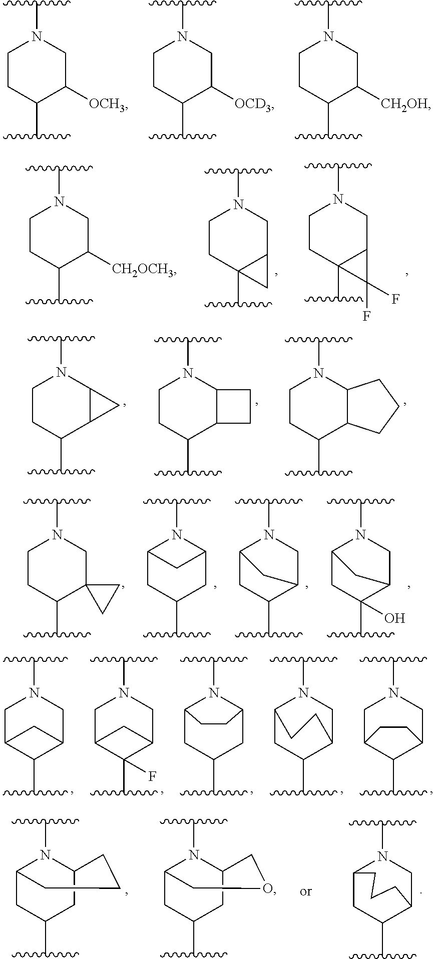

Provided herein as Embodiment 240 is the compound or salt of Embodiment 239, wherein X is

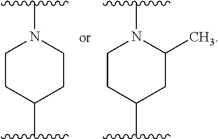

Provided herein as Embodiment 241 is the compound or salt of Embodiment 203, wherein X is

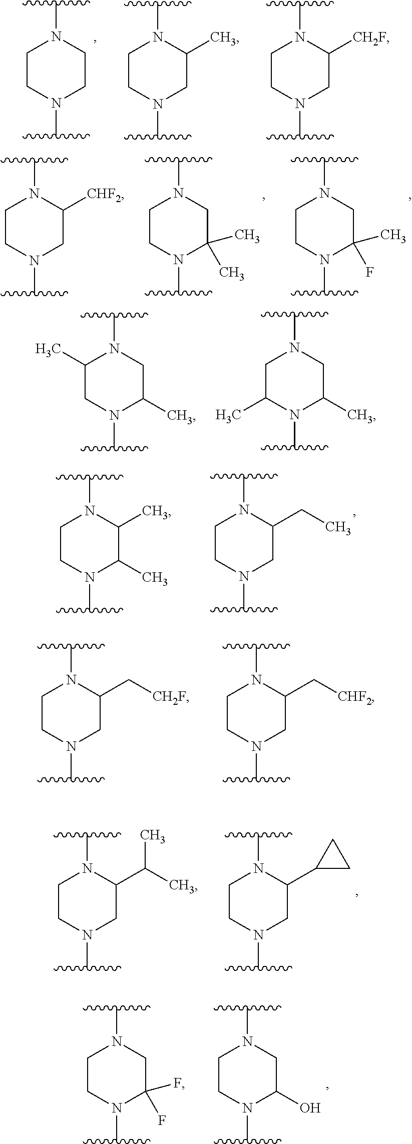

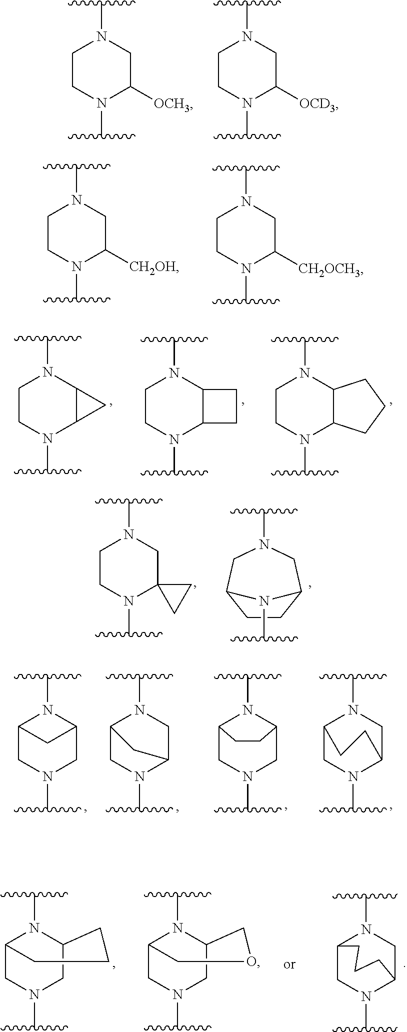

Provided herein as Embodiment 242 is the compound or salt of Embodiment 241, wherein X is

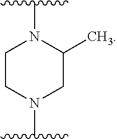 [SMILES: *N1CCN(*)C(C)C1]

Provided herein as Embodiment 243 is the compound or salt of any one of Embodiments 203-232, wherein X is

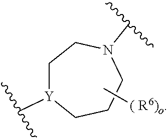

Provided herein as Embodiment 244 is the compound or salt of Embodiment 203, wherein X is

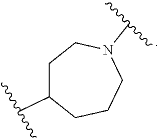 [SMILES: *C1CCCN(*)CC1]

Provided herein as Embodiment 245 is the compound or salt of any one of Embodiments 203-228, wherein X is

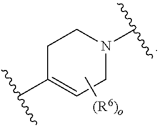

Provided herein as Embodiment 246 is the compound or salt of Embodiment 203, wherein X is

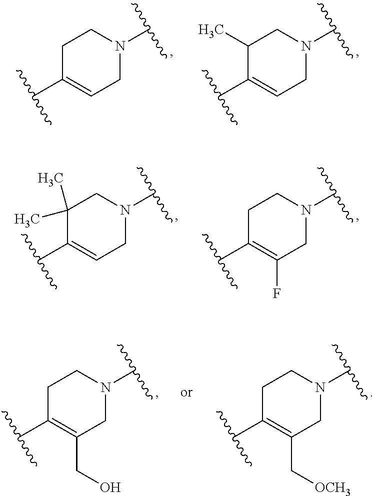

Provided herein as Embodiment 247 is the compound or salt of Embodiment 246, wherein X is

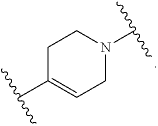 [SMILES: *C1=CCN(*)CC1]

Provided herein as Embodiment 248 is the compound or salt of Embodiment 203, wherein X is

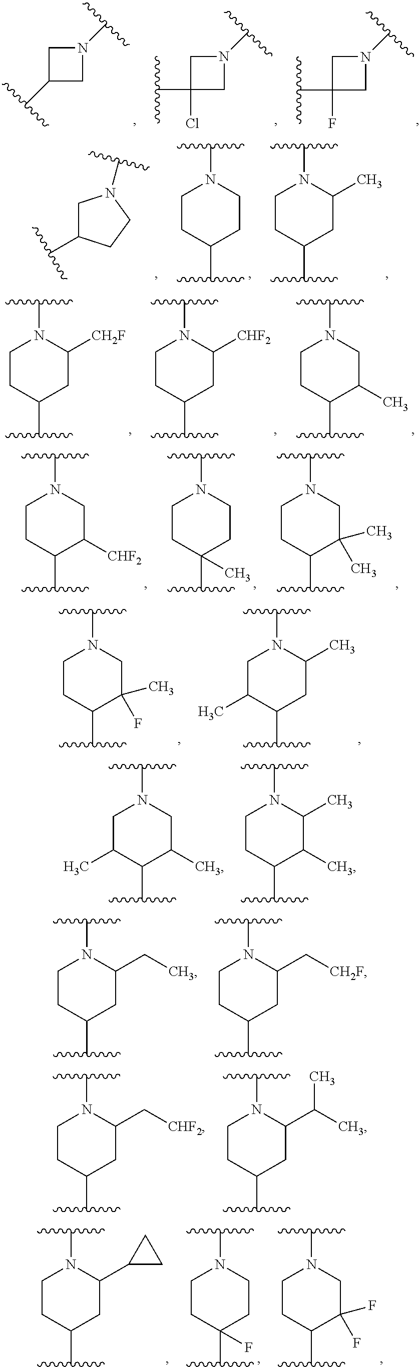

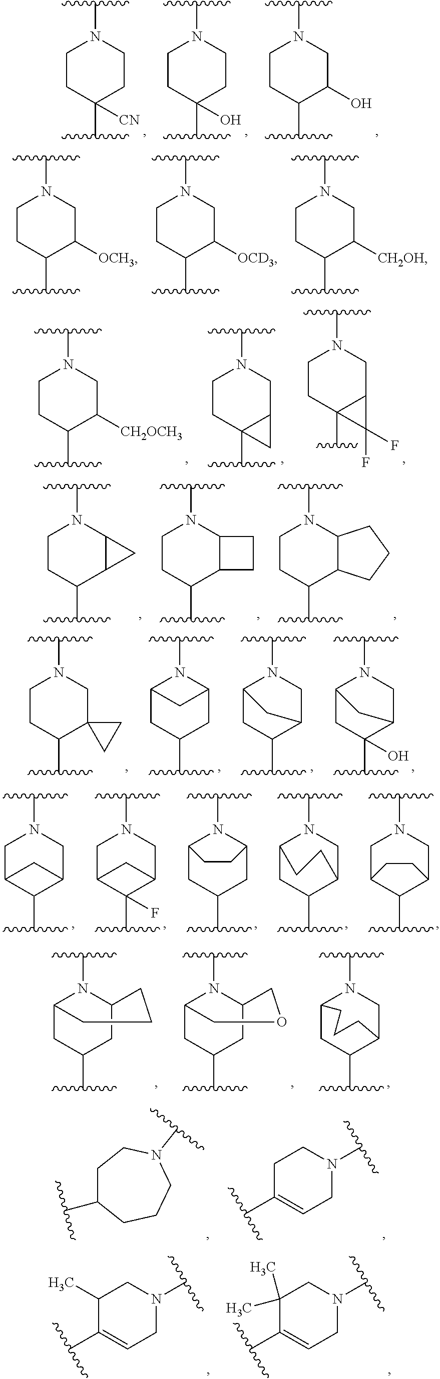

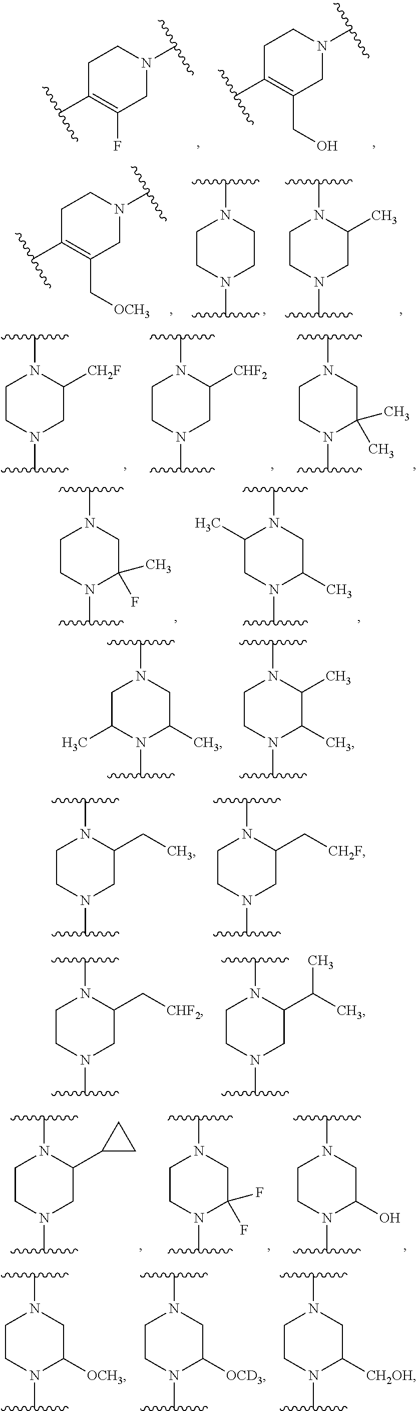

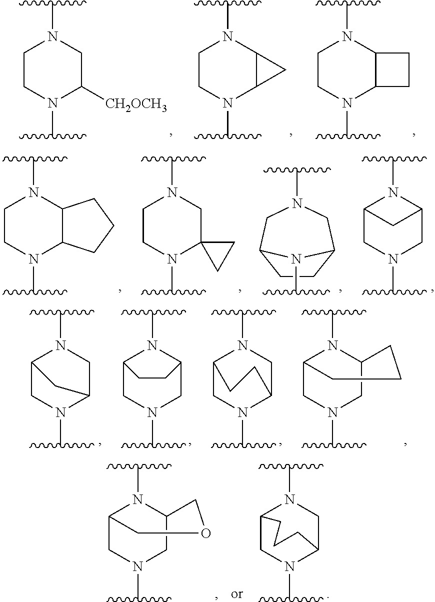

Provided herein as Embodiment 249 is the compound or salt of Embodiment 248, wherein X is

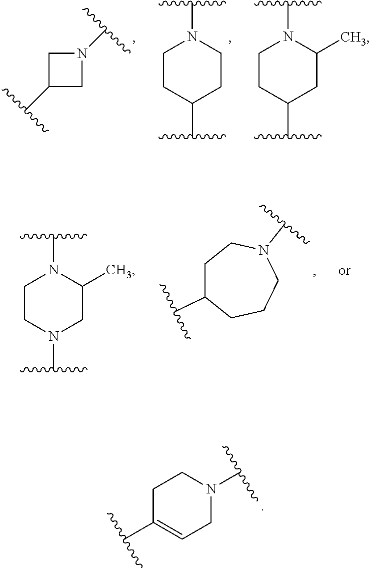

Provided herein as Embodiment 250 is the compound or salt of Embodiment 249, wherein X is

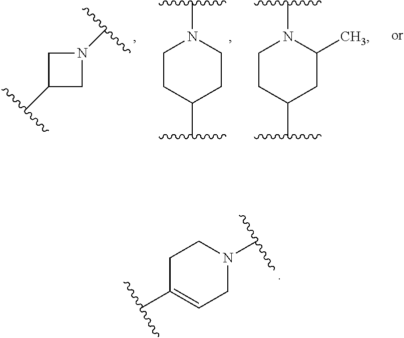

Provided herein as Embodiment 251 is the compound or salt of any one of Embodiments 58-250, wherein Z is unsubstituted phenyl or phenyl substituted with 1-4 substituents.

Provided herein as Embodiment 252 is the compound or salt of Embodiment 251, wherein each substituent independently is halo, C₀₋₃alkyleneCN, C₀₋₆alkyleneOH, C₀₋₆alkylene-C₁₋₄alkoxy, C₀₋₃alkylene-C₁₋₄thioalkyl, or

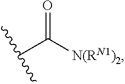

wherein each R^{N1} independently is H or CH₃.

Provided herein as Embodiment 253 is the compound or salt of Embodiment 252, wherein each substituent independently is F, Cl, CN, OCH₃, SCH₃, CH₂OH, or

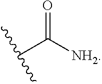 [SMILES: *C(N)=O]

Provided herein as Embodiment 254 is the compound or salt of any one of Embodiments 58-250, wherein Z is

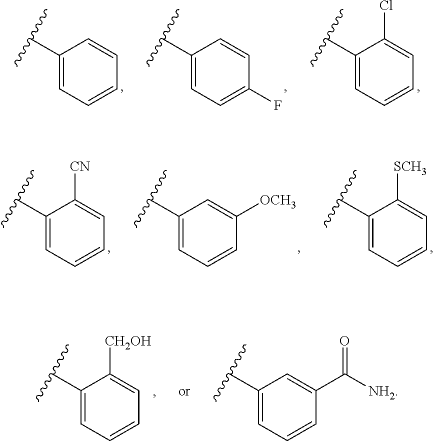

Provided herein as Embodiment 255 is the compound or salt of any one of Embodiments 58-250, wherein Z is heteroaryl comprising 5 or 6 total ring atoms and 1-3 heteroatoms selected from N, O, and S, wherein the heteroaryl is unsubstituted or substituted with 1 or more substituents.

Provided herein as Embodiment 256 is the compound or salt of Embodiment 255, wherein the heteroaryl is pyrrolyl, furanyl, thiophenyl, pyrazolyl, imidazolyl, thiazolyl, isothiazolyl, oxazolyl, isoxazolyl, triazolyl, thiadiazolyl, oxadiazolyl, pyridyl, pyridazinyl, pyrimidinyl, pyrazinyl, or triazinyl.

Provided herein as Embodiment 257 is the compound or salt of Embodiment 256, wherein the heteroaryl is pyrazolyl, imidazolyl, thiazolyl, isothiazolyl, oxazolyl, isoxazolyl, or triazolyl.

Provided herein as Embodiment 258 is the compound or salt of Embodiment 256, wherein the heteroaryl is pyridyl, pyridazinyl, pyrimidinyl, or pyrazinyl.

Provided herein as Embodiment 259 is the compound or salt of Embodiment 256, wherein the heteroaryl is pyrazolyl, thiazolyl, pyridyl, or pyridazinyl.

Provided herein as Embodiment 260 is the compound or salt of Embodiment 259, wherein the heteroaryl is pyrazolyl or pyridyl.

Provided herein as Embodiment 261 is the compound or salt of Embodiment 260, wherein the heteroaryl is pyrazolyl.

Provided herein as Embodiment 262 is the compound or salt of Embodiment 260, wherein the heteroaryl is pyridyl.

Provided herein as Embodiment 263 is the compound or salt of any one of Embodiments 255-262, wherein the heteroaryl is unsubstituted.

Provided herein as Embodiment 264 is the compound or salt of any one of Embodiments 255-262, wherein the heteroaryl is substituted with 1-4 substituents.

Provided herein as Embodiment 265 is the compound or salt of Embodiment 264, wherein each substituent independently is halo, CN, C₁₋₆alkyl, C₃₋₆haloalkyl, C₂₋₆alkenyl, C₂₋₅haloalkenyl, C₀₋₃alkylene-OH, C₀₋₆alkylene-C₁₋₃alkoxy, C₀₋₆alkylene-N(R^{N1})₂, C₀₋₂alkylene-C₃₋₆cycloalkyl, C₀₋₂alkylene-heterocycloalkyl having 3-6 total ring atoms and 1-3 heteroatoms selected from N, O, and S, or C₀₋₂alkylene-phenyl; wherein each of the C₁₋₃alkyl, C₂₋₆alkenyl, C₀₋₆alkylene-C₁₋₃alkoxy, C₃₋₇cycloalkyl, heterocycloalkyl, and phenyl substituents independently is optionally substituted with 1 or more further substituents, and each R^{N1} independently is H or C₁₋₃alkyl.

Provided herein as Embodiment 266 is the compound or salt of Embodiment 265, wherein each of the C₁₋₃alkyl, C₂₋₆alkenyl, C₀₋₆alkylene-C₁₋₃alkoxy, C₃₋₇cycloalkyl, heterocycloalkyl, and phenyl substituents independently is optionally substituted with 1-3 further substituents.

Provided herein as Embodiment 267 is the compound or salt of Embodiment 266, wherein each of the C₁₋₃alkyl, C₂₋₆alkenyl, C₀₋₆alkylene-C₁₋₃alkoxy, C₃₋₇cycloalkyl, heterocycloalkyl, and phenyl substituents independently is optionally substituted with 1 or 2 further substituents.

Provided herein as Embodiment 268 is the compound or salt of Embodiment 267, wherein each of the C₁₋₃alkyl, C₂₋₆alkenyl, C₀₋₆alkylene-C₁₋₃alkoxy, C₃₋₇ cycloalkyl, heterocycloalkyl, and phenyl substituents independently is optionally substituted with 1 further substituent.

Provided herein as Embodiment 269 is the compound or salt of Embodiment 265, wherein the C₁₋₃alkyl is CH₃, CH₂CH₃, CH₂CH₂CH₃, or CH(CH₃)₂, and each of the foregoing independently is optionally substituted with 1 or more further substituents.

Provided herein as Embodiment 270 is the compound or salt of Embodiment 269, wherein the C₁₋₆alkyl is CH₃, and the CH; is unsubstituted or substituted with 1 or more further substituents.

Provided herein as Embodiment 271 is the compound or salt of Embodiment 265, wherein the C₂₋₆alkenyl is CH═CH₂, CH₂CH═CH₂, or CH═CHCH₃, and each of the foregoing independently is optionally substituted with 1 or more further substituents.

Provided herein as Embodiment 272 is the compound or salt of Embodiment 265, wherein the C₀₋₃alkylene-C₁₋₃alkoxy is OCH₃, CH₂OCH₃, CH₂CH₂OCH₃, CH₂CH₂OCH—CH₃. CH₂CH₂CH₂OCH₃, CH(CH₃)OCH₃, CH(CH₃)CH₂OCH₃, CH(OCH₃)CH₂OCH₃, CH(CH₃)(OCH₃)CH₂OCH₃, C(CH₃)₂OCH₃, C(CH₃)₂CH₂OCH₃, CH₂CH(CH₃)OCH₃, CH₂(CH₃)(OCH₃)OCH₃, CH₂C(CH₃)₂OCH₃, or CH₂C(CH₃)₂OCH₃, and each of the foregoing independently is optionally substituted with 1 or more further substituents.

Provided herein as Embodiment 273 is the compound or salt of Embodiment 272, wherein the C₀₋₆alkylene-C₁₋₃alkoxy is CH(CH₃)OCH₃ or CH₂CH₂OCH₃, and each of the foregoing independently is optionally substituted with 1 or more further substituents.

Provided herein as Embodiment 274 is the compound or salt of Embodiment 265, wherein the cycloalkyl of the C₀₋₃alkylene-C₃₋₅cycloalkyl is cyclopropyl, cyclobutyl, cyclopentyl, or cyclohexyl, and each of the foregoing independently is optionally substituted with 1 or more further substituents.

Provided herein as Embodiment 275 is the compound or salt of Embodiment 274, wherein the cycloalkyl of the C₀₋₂alkylene-C₃₋₆cycloalkyl is cyclopropyl or cyclobutyl, and each of the foregoing independently is optionally substituted with 1 or more further substituents.

Provided herein as Embodiment 276 is the compound or salt of Embodiment 265, wherein the heterocycloalkyl of the C₀₋₂alkylene-heterocycloalkyl is azetidinyl, pyrrolidinyl, piperidinyl, pyrazolidinyl, oxetanyl, tetrahydrofuranyl, tetrahydropyranyl, or morpholinyl, and each of the foregoing independently is optionally substituted with 1 or more further substituents.

Provided herein as Embodiment 277 is the compound or salt of Embodiment 276, wherein the heterocycloalkyl of the C₀₋₂alkylene-heterocycloalkyl is azetidinyl, oxetanyl, tetrahydrofuranyl, tetrahydropyranyl, or morpholinyl, and each of the foregoing is optionally substituted with 1 or more further substituents.

Provided herein as Embodiment 278 is the compound or salt of Embodiment 277, wherein the heterocycloalkyl of the C₀₋₂alkylene-heterocycloalkyl is azetidinyl or oxetanyl, and each of the foregoing is optionally substituted with 1 or more further substituents.

Provided herein as Embodiment 279 is the compound or salt of Embodiment 265, wherein at least one substituent is Br, Cl, F, or CN.

Provided herein as Embodiment 280 is the compound or salt of Embodiment 265, wherein the C₁₋₃haloalkyl is CF₃, CHF₂, CH₂F, CH₂CHF₂, CH₂CH₂F, CH(CH₂F)₂, CH(CH₃)CH₂F, or CH(CH₃)CHF₂.

Provided herein as Embodiment 281 is the compound or salt of Embodiment 265, wherein the C₁₋₆haloalkenyl is C(═CH)CH₂F.

Provided herein as Embodiment 282 is the compound or salt of Embodiment 265, wherein C₀₋₆alkylene-OH is OH, CH₂OH, CH₂CH₂OH, CH(CH,)CH₂OH, C(CH₃)₂OH, C(CH₃)₂CH₂OH, or CH₂C(CH₃)₂OH.

Provided herein as Embodiment 283 is the compound or salt of Embodiment 265, wherein the C₀₋₃alkylene-N(R^{N1})₂, is NH₂, CH₂NH₂, CH₂ NHCH₃, CH₂N(CH₃)₂, CH₂CH₂NH₂, CH₂CH₂NHCH₃, or CH₂CH₂N(CH₃)₂.

Provided herein as Embodiment 284 is the compound or salt of any one of Embodiments 265-278, wherein each further substituent independently is D, halo, C₁₋₃alkyl, C₁₋₃haloalkyl, C₁₋₂alkyleneOH, C₁₋₂alkylene-C₁₋₃alkoxy, C₁₋₃deuterated alkoxy, N(R^{N1})₂, (C═O) C₁₋₃alkyl, C₃₋₅cycloalkyl, or heterocycloalkyl having 3-5 total ring atoms and 1 or 2 heteroatoms selected from N, O, and S; or two geminal further substituents, together with the atom to which they are attached, form spiro-C₃₋₅cycloalkyl or spiro-heterocycloalkyl having 3-5 total ring atoms and 1 or 2 heteroatoms selected from N, O, and S; or two vicinal further substituents, together with the atoms to which they are attached, form fused-C₃₋₅cycloalkyl or fused-heterocycloalkyl having 3-5 total ring atoms and 1 or 2 heteroatoms selected from N, O, and S; wherein each of the foregoing cycloalkyl and heterocycloalkyl groups independently is unsubstituted or substituted with halo, C₁₋₄alkyl, or a combination thereof, and each R^{N1} independently is H or C₁₋₃alkyl.

Provided herein as Embodiment 285 is the compound or salt of Embodiment 284, wherein each further substituent independently is D, Br, Cl, F, OH, CH₃, CF₃, CF₂H, CFH₂, OCH₃, OCD₃, CH₂OCH₃, N(CH₃)₂, (C═O)CH₃, oxetanyl, or azetidinyl; or two geminal further substituents, together with the atom to which they are attached, form spiro-oxetanyl, or spiro-azetidinyl; wherein each of the foregoing oxetanyl, azetidinyl, spiro-oxetanyl, and spiro-azetidinyl is unsubstituted or substituted with F, CH₃, or a combination thereof.

Provided herein as Embodiment 286 is the compound or salt of Embodiment 285, wherein each further substituent independently is D, Br, Cl, F, OH, CH₃, CF₃, CF₂H, CFH₂, OCH₃, OCD₃, N(CH₃)₂, (C═O)CH₃,

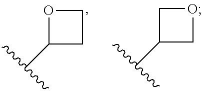 [SMILES: *C1CCO1 ; *C1COC1]

or two geminal further substituents, together with the atom to which they are attached, form

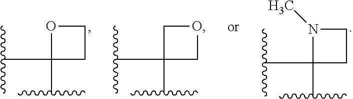

Provided herein as Embodiment 287 is the compound or salt of Embodiment 286, wherein each further substituent independently is D, CH₃, OCH₃, OCD₃, N(CH₃)₂,

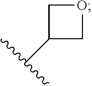 [SMILES: *C1COC1]

or two geminal further substituents, together with the atom to which they are attached, form

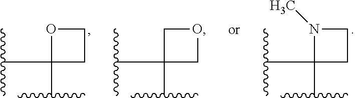

Provided herein as Embodiment 288 is the compound or salt of Embodiment 264, wherein each substituent of the heteroaryl of Z independently is Cl, F, CN, CH₃, CD₃, CH₂CH₃, CH(CH₃)₂, CF₃, CHF₂, CH₂F, CH₂CHF₂, CH₂CH₂F, CH(CH₂F)₂, CH(CH₃)CH₂F, CH(CH₃)CHF₂, C(═CH₂)CH₂F, OH, CH₂OH, CH₂CH₂OH, CH(CH₃)CH₂OH, C(CH₃)₂OH, C(CH₃)₂CH₂OH, CH₂C(CH₃)₂OH, OCH₃, OCD₃, CH₂OCH₃, CH₂OCD₃, CH₂CH₂OCH₃, CHFCH₂OCH₃, CF₂CH₂OCH₃, CH₂CH₂OCD₃, CHCH₂OCH₂CH₃, CH₂CH₂CH₂OCH₃, CH₂CH₂CH₂OCD₃, CH(CH₃)OCH₃, CH(CH₃)CH₂OCH₃, CH(CH₃)CH₂OCD₃, C(CH₃)₂CH₂OCH₃, C(CH₃)₂CH₂OCD₃, CH—CH(CH)OCH₃, CH₂CH(CH₃)OCD₃, CH₂C(CH₃)₂OCH₃, CH₂C(CH₃),OCD₃, NH₂, CH₂NH₂, CH₂NHCH₃, CH₂N(CH₃)₂, CH—CH₂NH₂, CH₂CH₂NHCH₃, CH₂CH₂N(CH₃)₂,

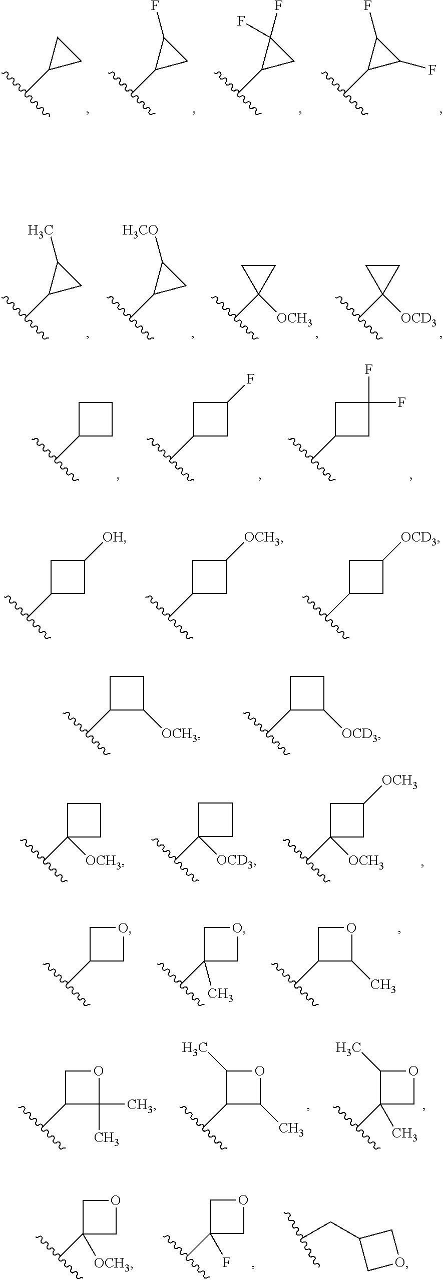

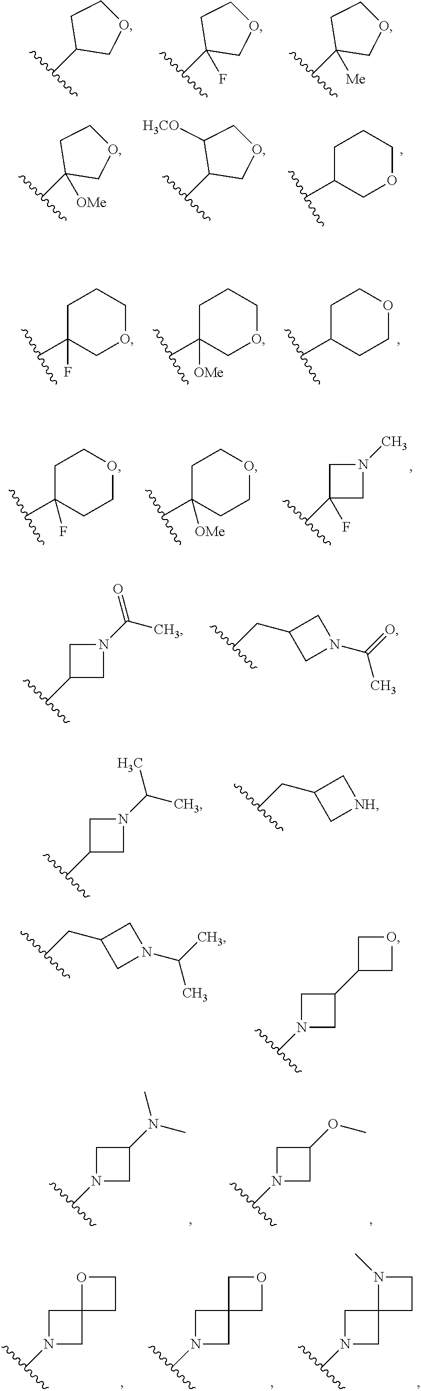

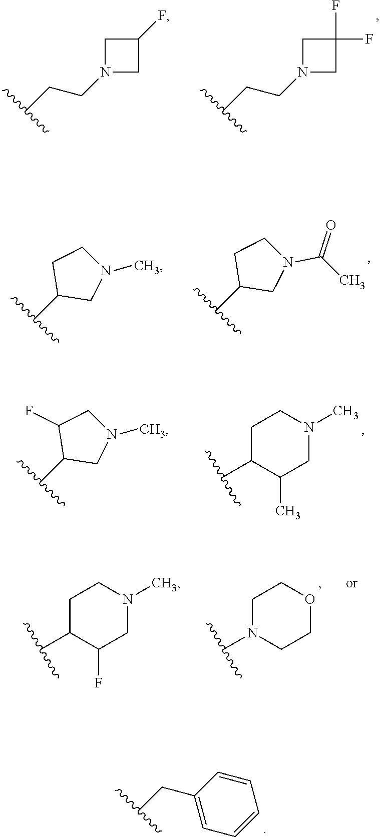

Provided herein as Embodiment 289 is the compound or salt of Embodiment 288, wherein each substituent of the heteroaryl of Z independently is CH₃, C(CH₃)₂CH₂OH, CH₂CH₂OCH₃, CH₂CH₂OCD₃, CH(CH₃)OCH₃,

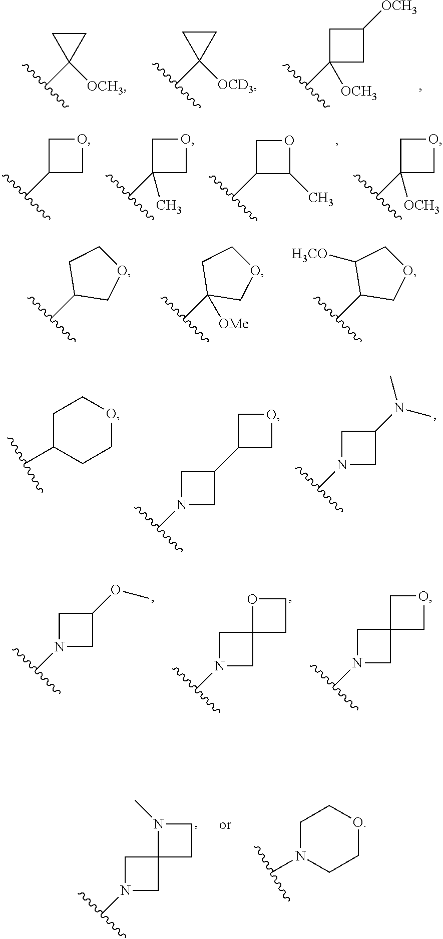

Provided herein as Embodiment 290 is the compound or salt of Embodiment 289, wherein each substituent of the heteroaryl of Z independently is CH₃, CH₂CH₂OCH;

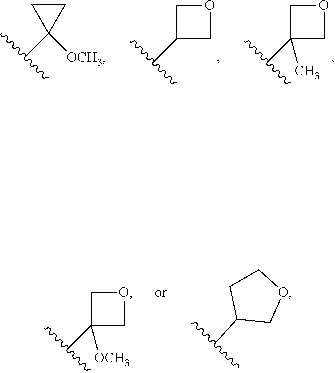

or any combination of the foregoing.

Provided herein as Embodiment 291 is the compound or salt of any one of Embodiments 58-250, wherein Z is

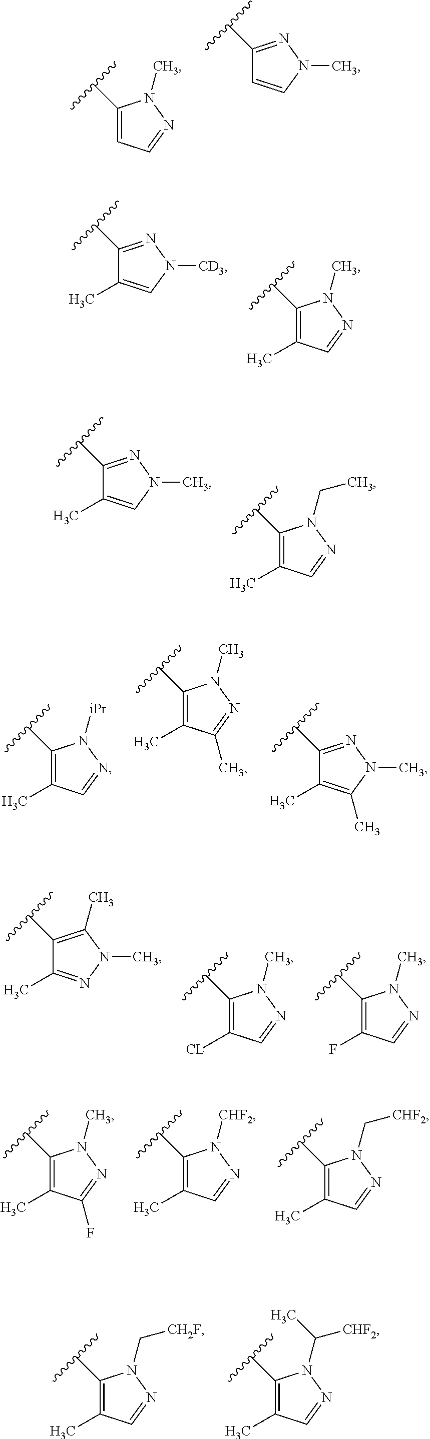

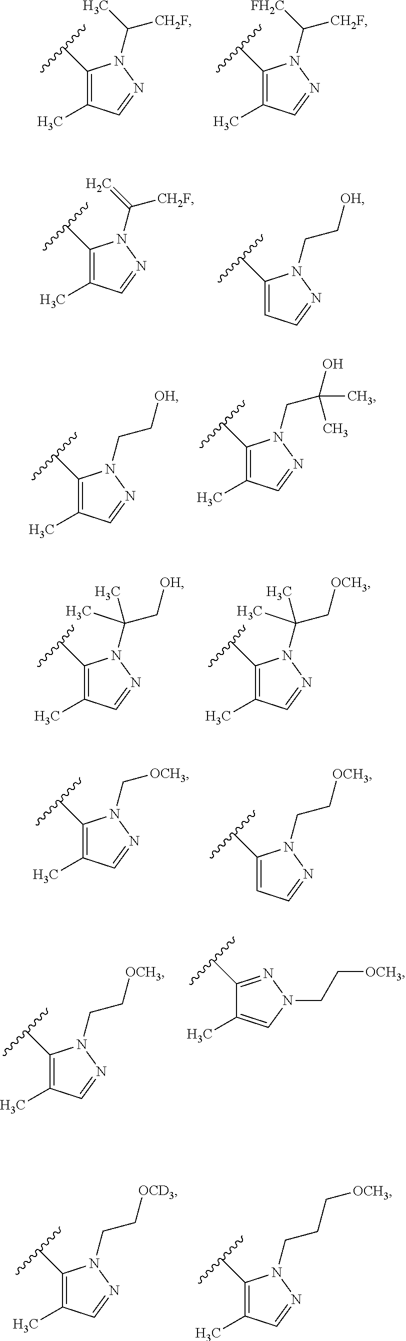

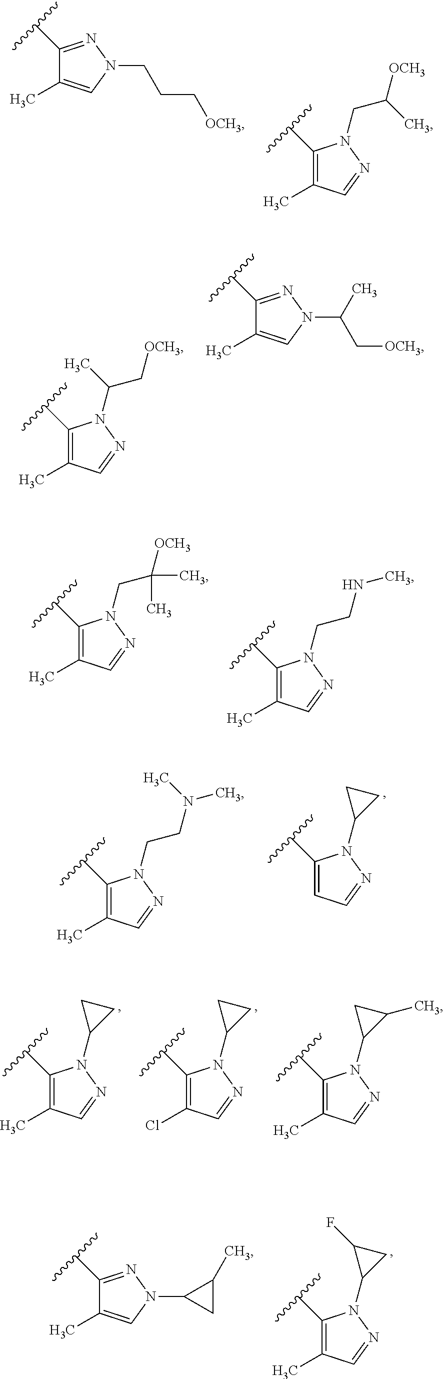

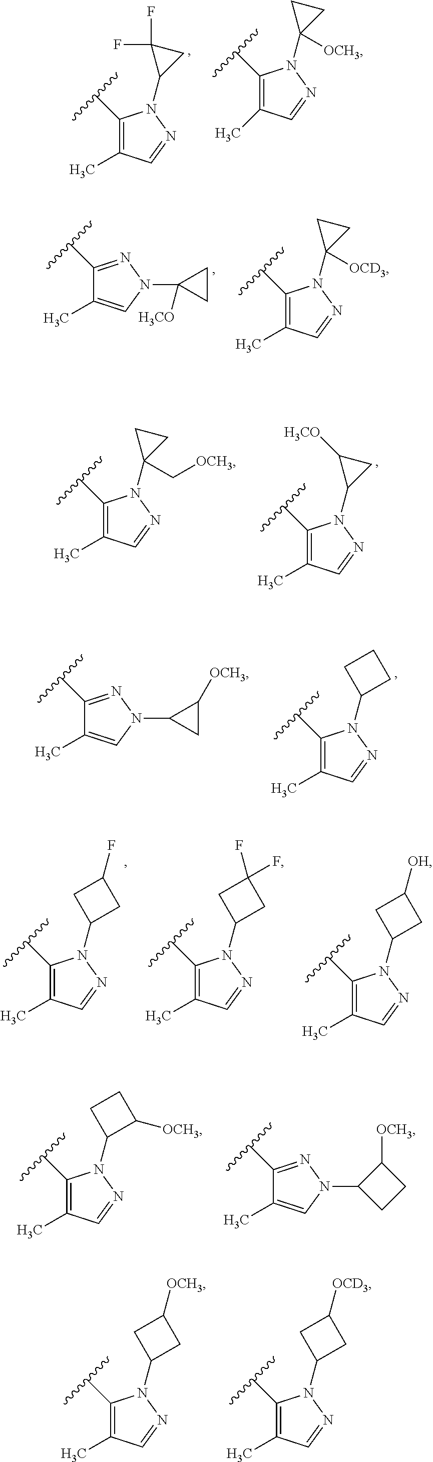

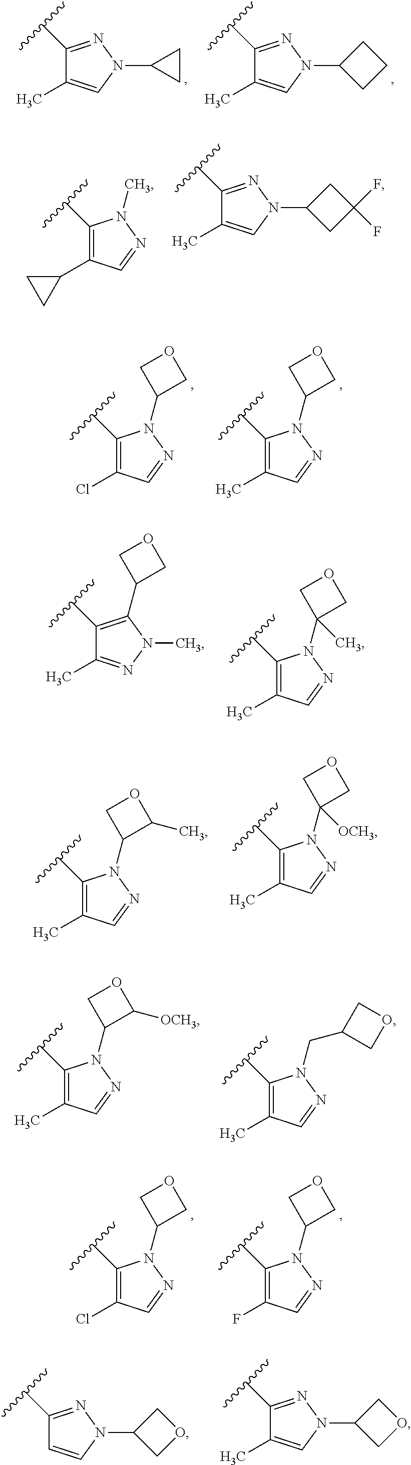

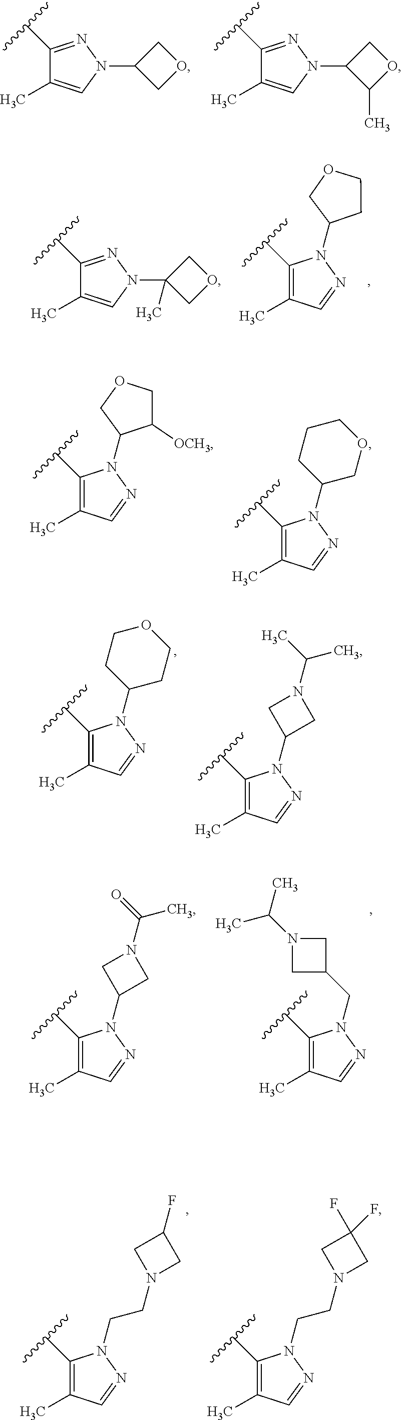

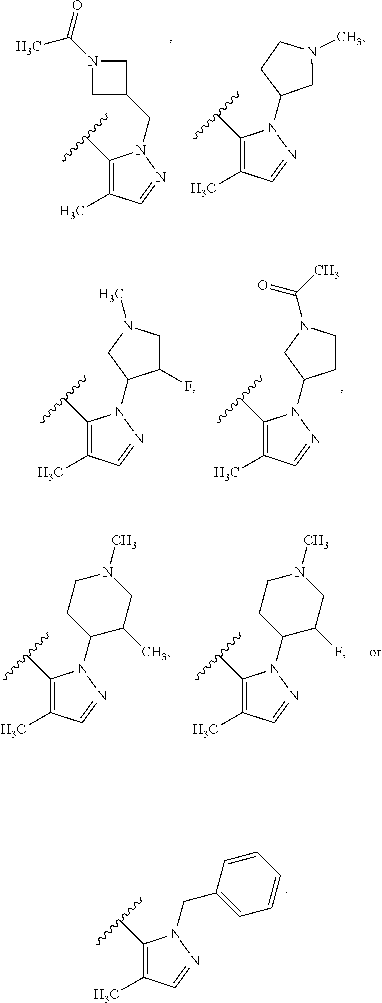

Provided herein as Embodiment 292 is the compound or salt of Embodiment 291, wherein Z is

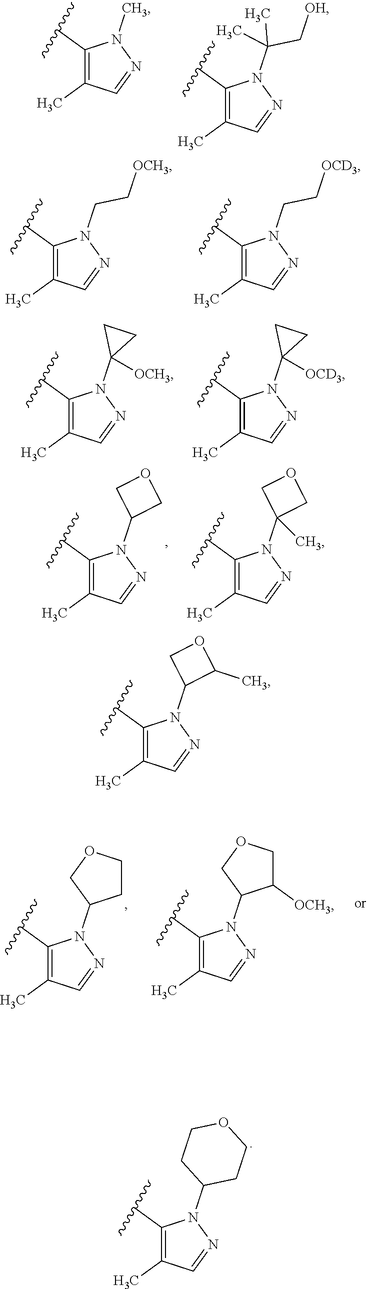

Provided herein as Embodiment 293 is the compound or salt of any one of Embodiments 58-250, wherein Z is

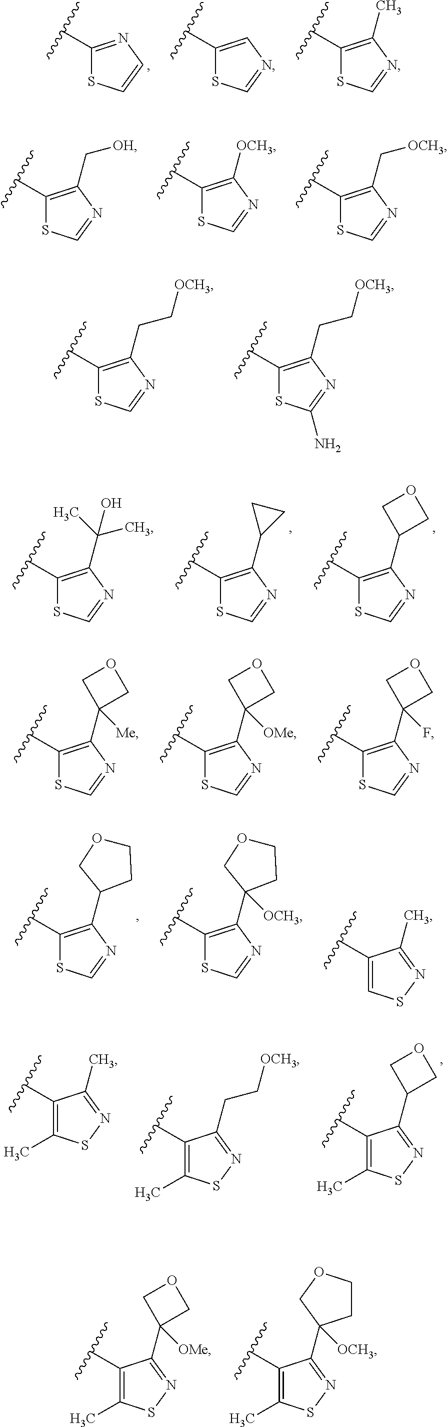

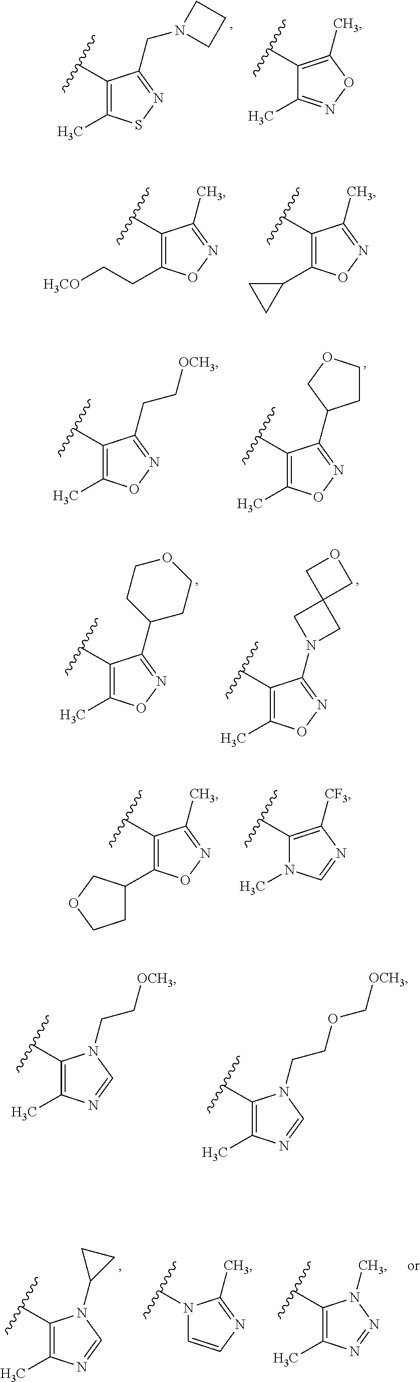

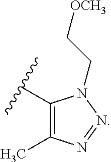 [SMILES: *c1c(C)nnn1CCOC]

Provided herein as Embodiment 294 is the compound or salt of any one of Embodiments 58-250, wherein Z is

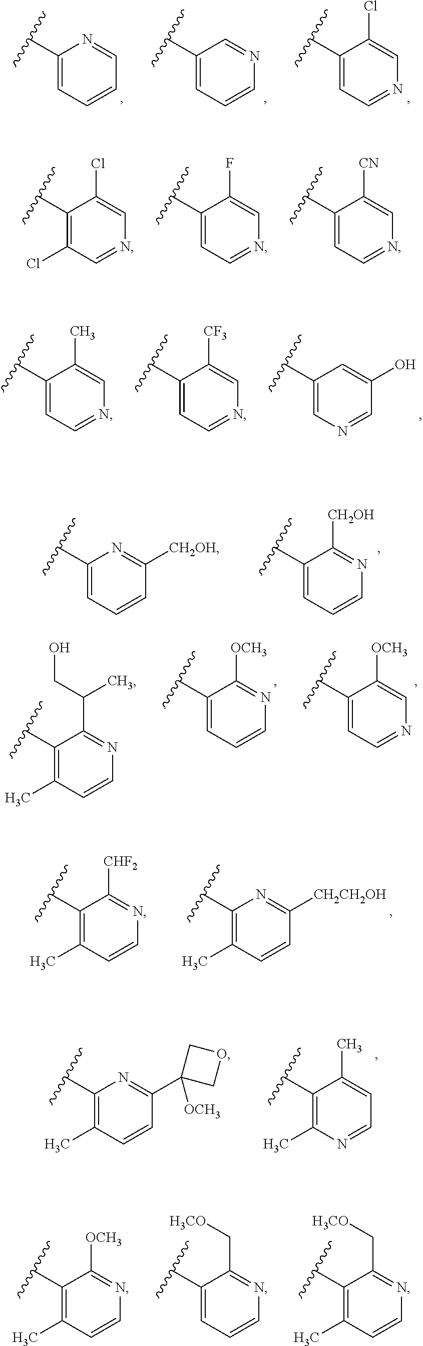

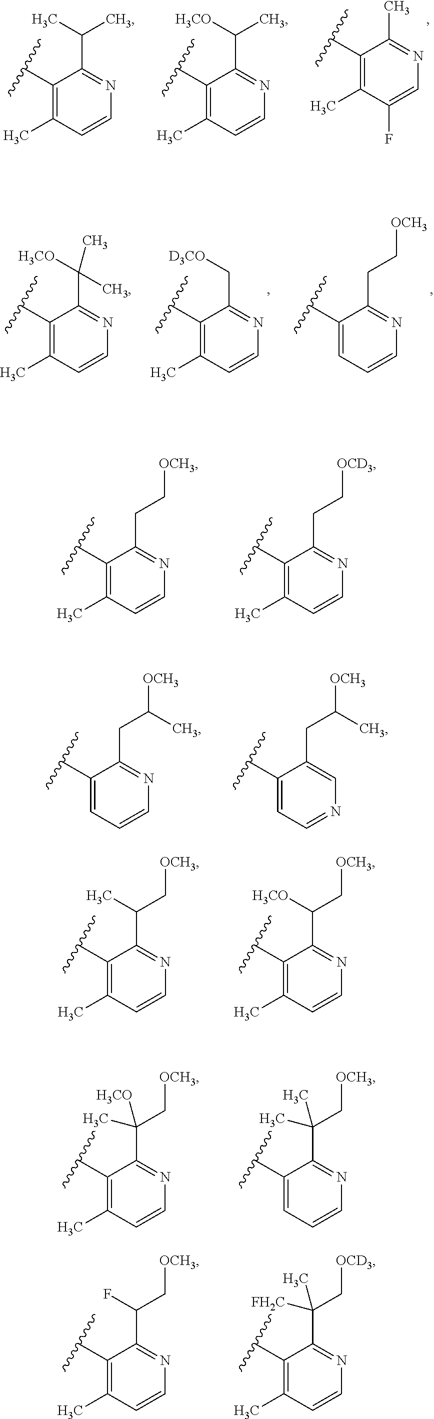

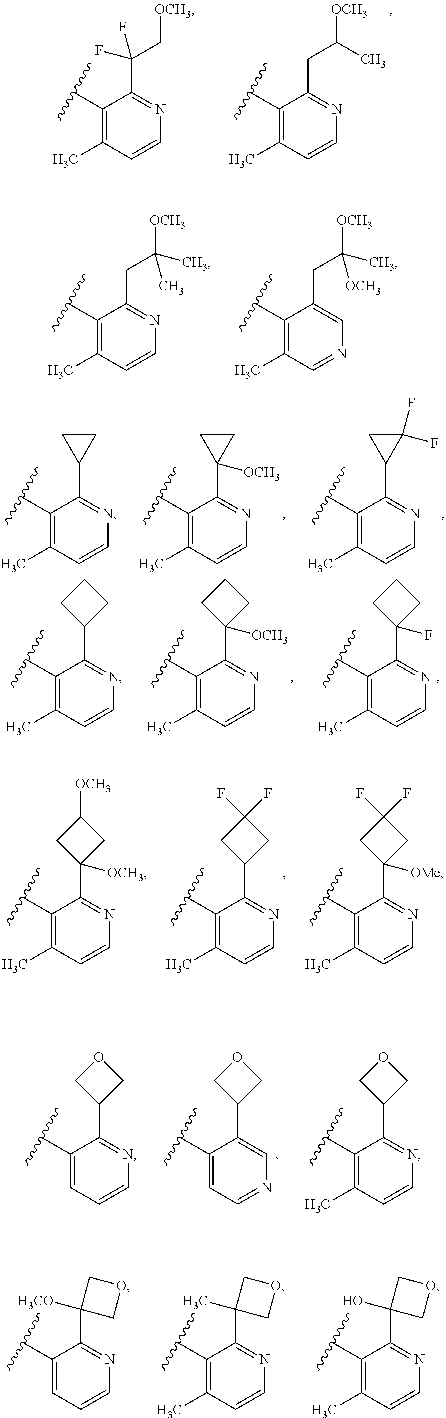

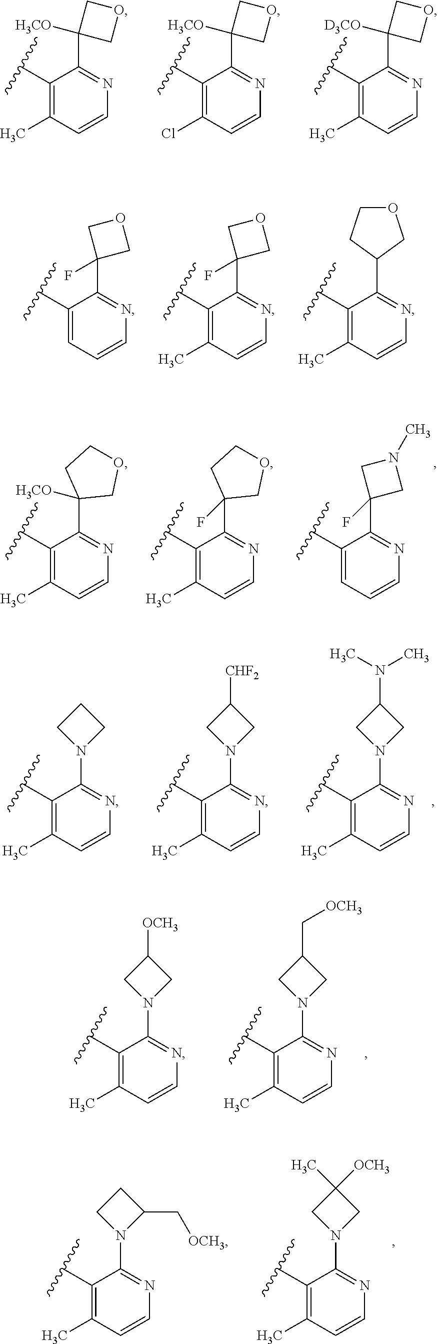

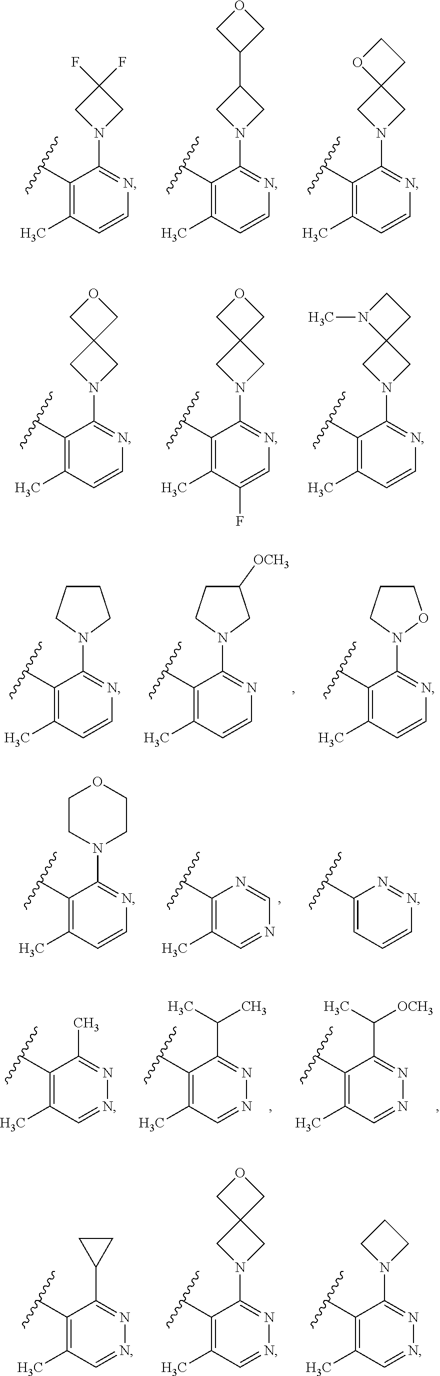

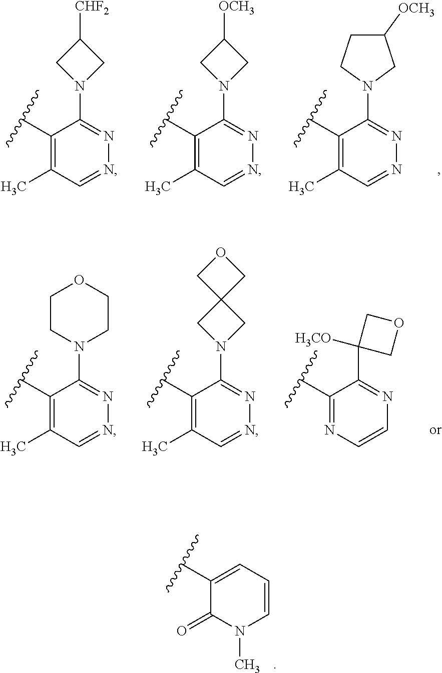

Provided herein as Embodiment 295 is the compound or salt of Embodiment 295, wherein Z is

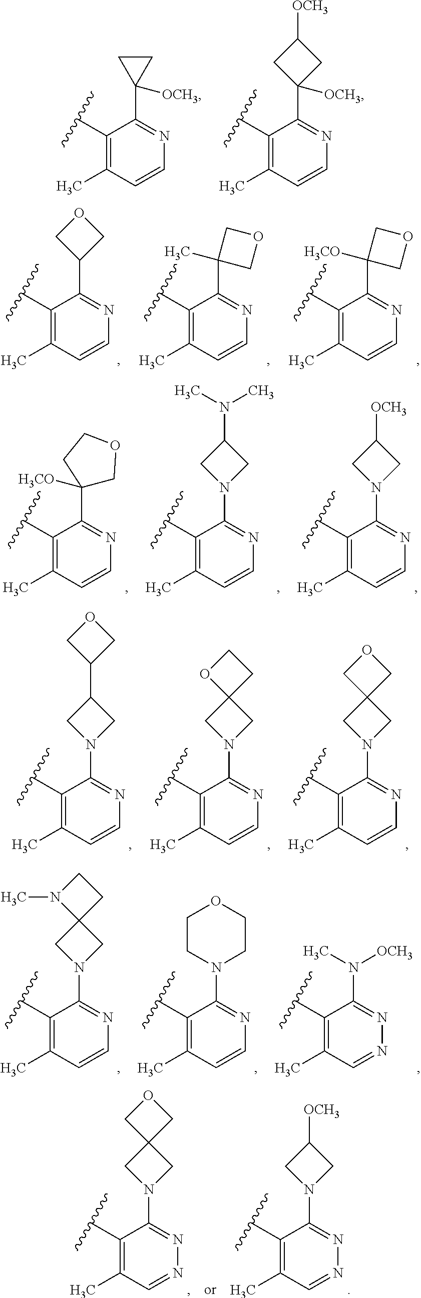

Provided herein as Embodiment 296 is the compound or salt of any one of Embodiments 58-250, wherein Z is

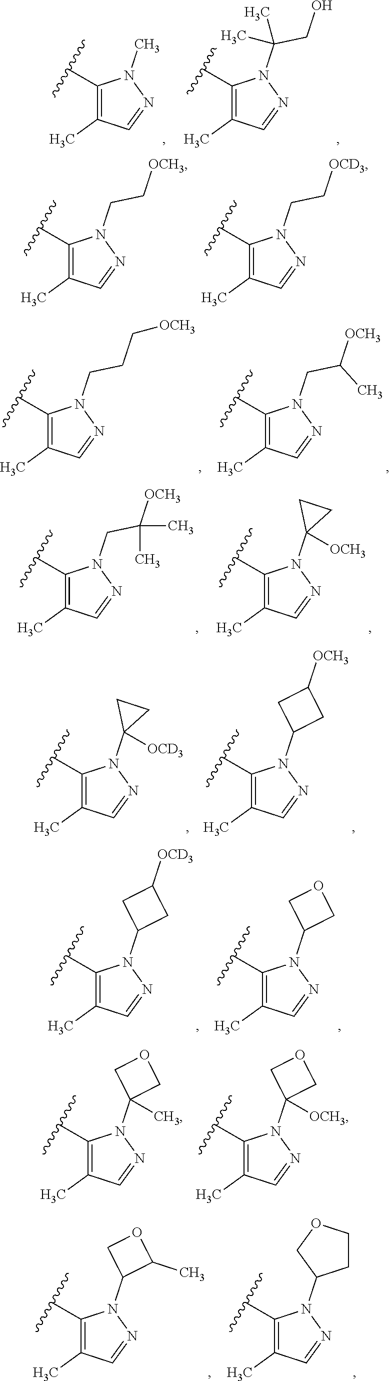

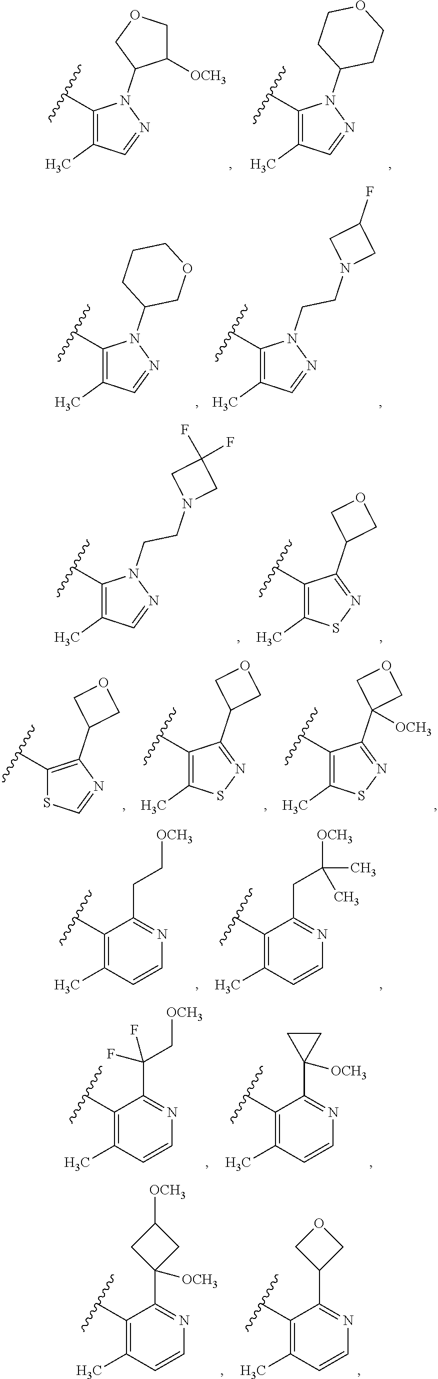

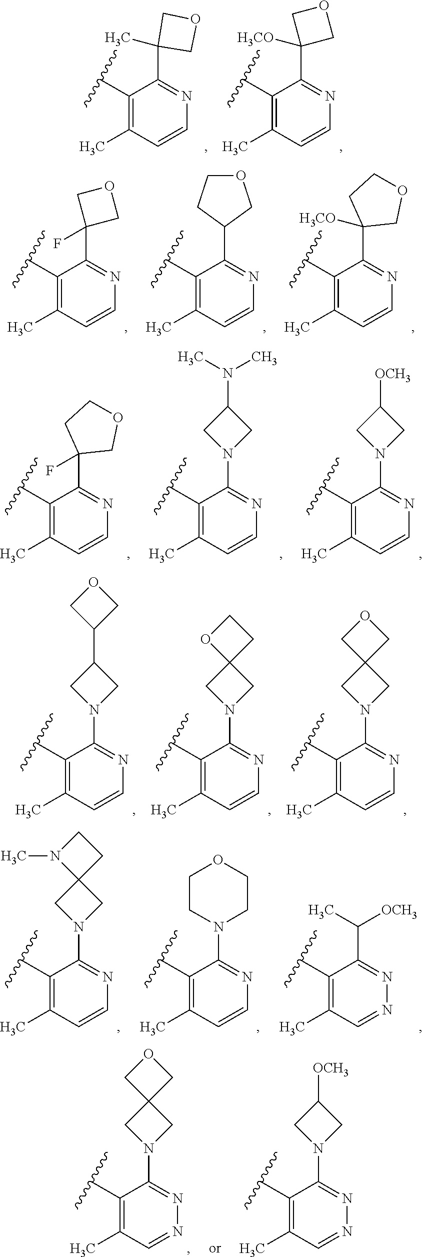

Provided herein as Embodiment 297 is the compound or salt of Embodiments 296, wherein Z is

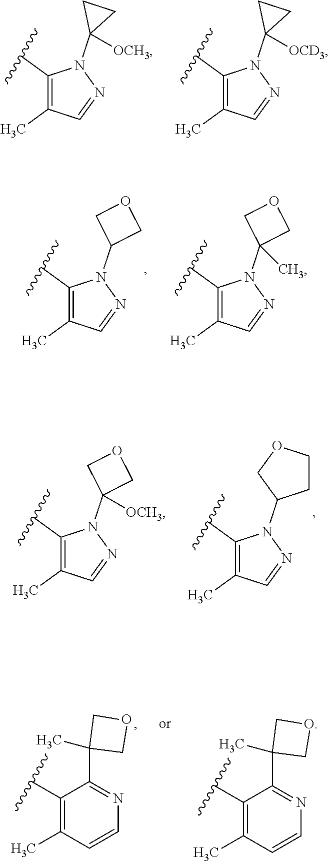

Provided herein as Embodiment 298 is the compound or salt of any one of Embodiments 58-250, wherein Z is a bicyclic ring comprising heteroaryl having 5 or 6 total ring atoms and 1-3 heteroatoms selected from N, O, and S fused to C₅₋₆cycloalkyl ring or a heterocycloalkyl ring having 5 or 6 total ring atoms and 1 or 2 heteroatoms selected from N, O, and S; wherein the bicyclic ring is unsubstituted or substituted with 1-4 substituents.

Provided herein as Embodiment 399 is the compound or salt of Embodiment 299, wherein the heteroaryl is pyridyl, pyridazinyl, pyrimidinyl, or pyrazinyl; and the fused ring has 5 total atoms and 1 oxygen atom in the fused ring, 5 total atoms and 1 nitrogen atom in the fused ring, 6 total atoms and 1 nitrogen or oxygen atom in the ring, or 6 total atoms, 1 oxygen atom, and 1 nitrogen atom in the fused ring.

Provided herein as Embodiment 300 is the compound or salt of Embodiment 299 or 300, wherein the bicyclic ring is substituted with halo, CN, C₁₋₃alkyl, C₁₋₆haloalkyl, C₀₋₆alkylene-OH, or C₀₋₃alkylene-C₁₋₃alkoxy, or any combination of the foregoing.

Provided herein as Embodiment 301 is the compound or salt of Embodiment 300, wherein each substituent of the bicyclic ring independently is Br, Cl, F, CN, CH₃, CH₂CH₃, CH₂CH₂CH₃, CH(CH₃)₂, CF₃, CHF₂, CH₂F, CH₂CHF₂, CH₂CH₂F, CH(CH₂F)₂, CH(CH₃)CH₂F, CH(CH₃)CHF₂), OH, CH₂OH, CH₂CH₂OH, CH(CH₃)CH₂OH, C(CH₃)₂OH, C(CH₃)₂CH₂OH, CH₂C(CH₃)₂OH, OCH₃, CH₂OCH₃, CH₂CH₂OCH₃, CH₂CH₂CH₂OCH₃, CH(CH)CH₂OCH₃, C(CH₃)₂CH₂OCH₃, CH₂CH(CH₃)OCH₃, or CH₂C(CH₃)₂OCH₃.

Provided herein as Embodiment 302 is the compound or salt Embodiments 299 or 300, wherein Z is

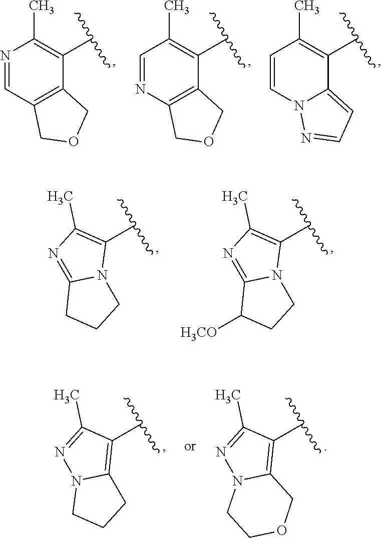

Provided herein as Embodiment 303 is the compound or salt of Embodiment 58, wherein: m is 0 or 1; n is 0; o is 0 or 1; A is N; W¹ is CH; W² is N; X is

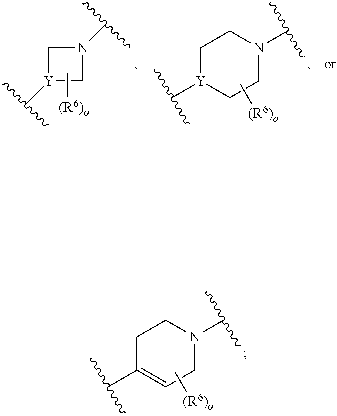

Y is CH or N; Z is heteroaryl comprising 5 or 6 total ring atoms and 1-3 heteroatoms selected from N, O, and S, wherein the heteroaryl is substituted with 1 or more substituents; each of R^{1a}, R^{1b} and R² is H; R³ is CH₃; and R⁵ is CH₂F, CHF₂, or CF₃

Provided herein as Embodiment 304 is the compound or salt of Embodiment 303, wherein X is

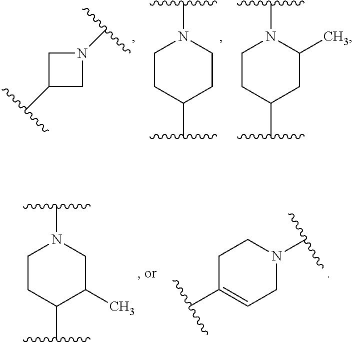

Provided herein as Embodiment 305 is the compound or salt of Embodiment 304, wherein X is

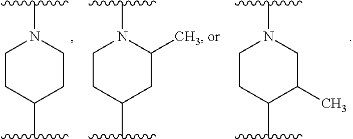

Provided herein as Embodiment 306 is the compound or salt of any one of Embodiments 303-305, wherein [symbol] is

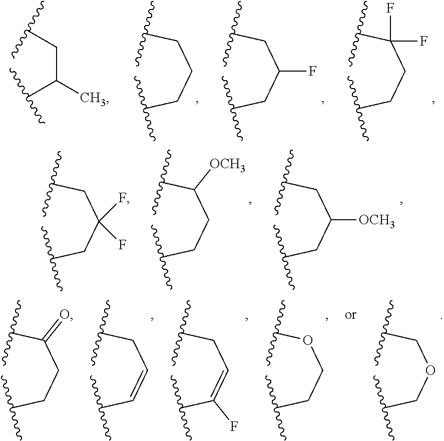

Provided herein as Embodiment 307 is the compound or salt of Embodiment 306, wherein [symbol] is

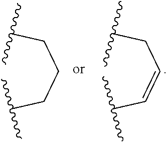 [SMILES: *CCC* ; */C=C\C*]

Provided herein as Embodiment 308 is the compound or salt of any one of Embodiments 303-307, wherein the heteroaryl of Z is pyrazolyl, thiazolyl, pyridyl or pyridazinyl, wherein each of the foregoing is substituted with 1 or 2 substituents.

Provided herein as Embodiment 309 is the compound or salt of Embodiment 308, wherein the heteroaryl of Z is pyrazolyl or pyridyl, and each of the foregoing is substituted with 2 substituents.

Provided herein as Embodiment 310 is the compound or salt of Embodiment 308 or 309, wherein each substituent independently is C₁₋₆alkyl, C₀₋₂alkylene-C₃₋₆cycloalkyl. C₀₋₂alkylene-heterocycloalkyl having 3-6 total ring atoms and 1-3 heteroatoms selected from N, O, and S, or a combination of the foregoing, wherein the cycloalkyl and heterocycloalkyl is optionally substituted with 1 or 2 further substituents, and each further substituent independently is D, CH₃, OCH₃, OCD₃, N(CH₃)₂,

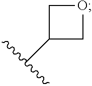 [SMILES: *C1COC1]

or two geminal further substituents, together with the atom to which they are attached, form

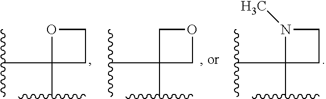

Provided herein as Embodiment 311 is the compound or salt of any one of Embodiments 303-307, wherein Z is

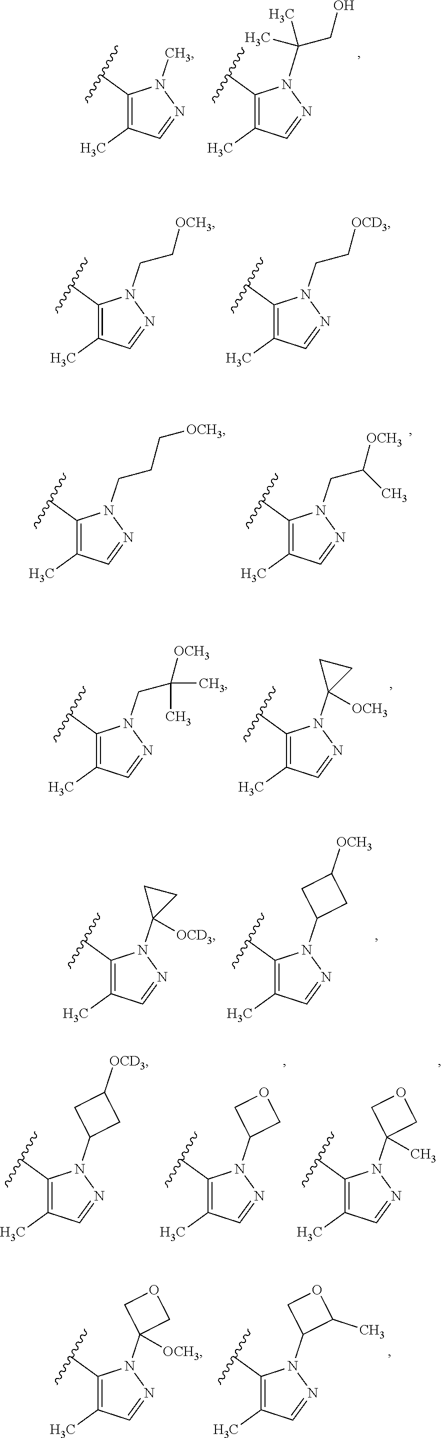

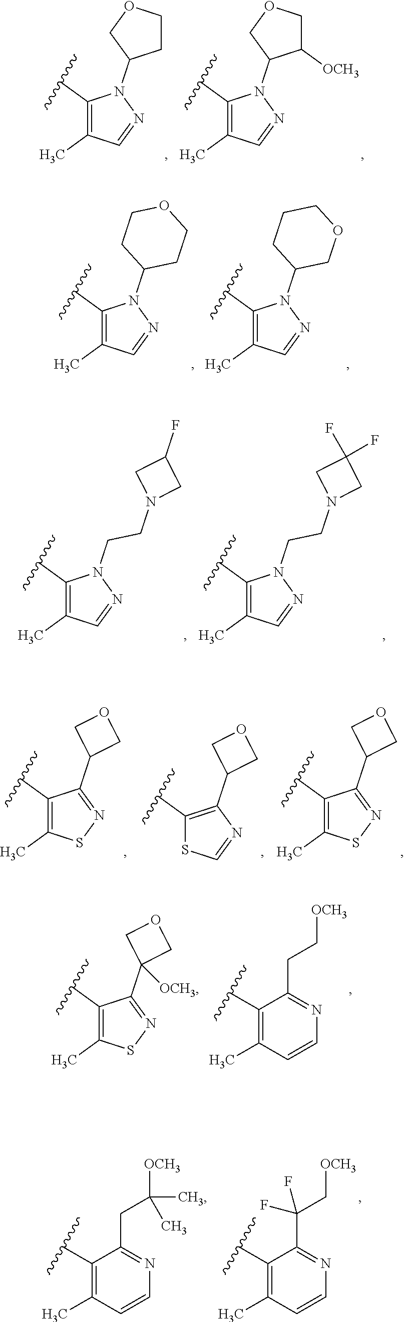

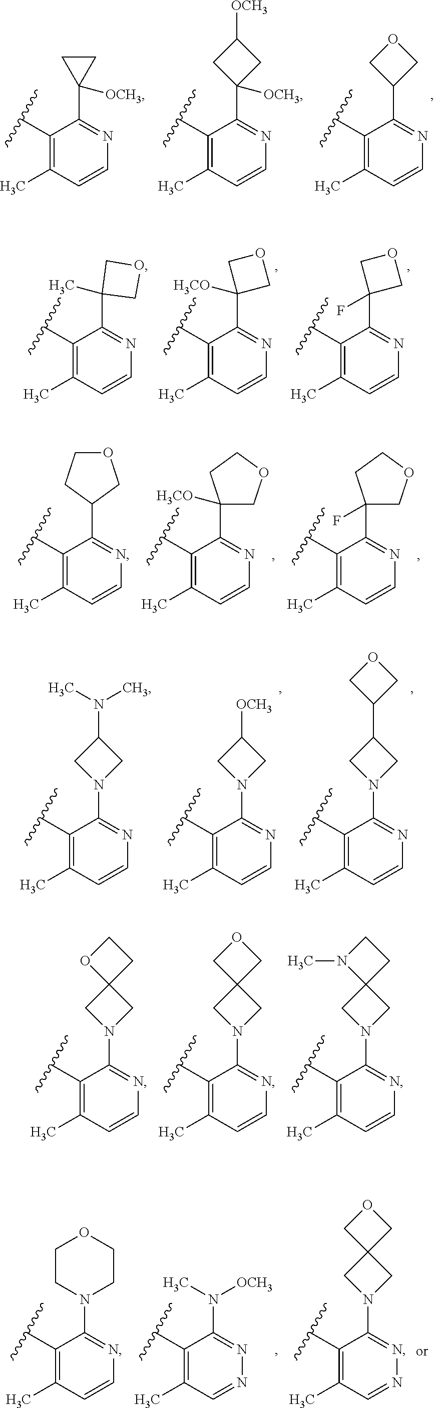

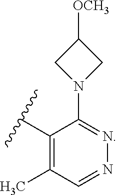 [SMILES: *c1c(C)cnnc1N1CC(OC)C1]

Provided herein as Embodiment 312 is the compound or salt of Embodiment 311, wherein Z is

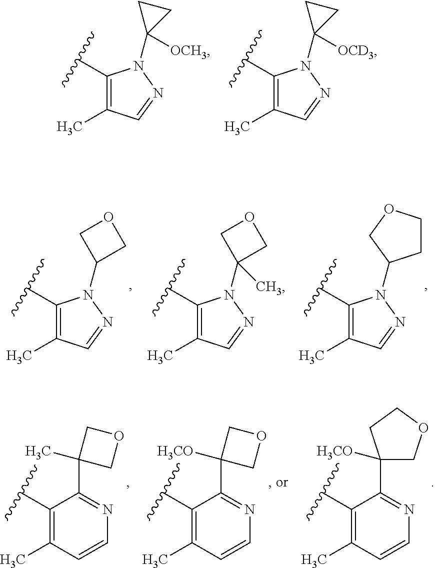

Provided herein as Embodiment 313 is the compound of Embodiment 58, wherein Formula (II) has a structure of Formula (IIA):

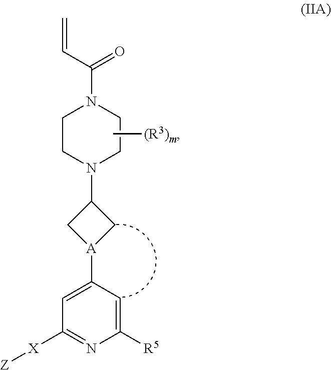

or a pharmaceutically acceptable salt thereof.

Provided herein as Embodiment 314 is the compound of Embodiment 313, wherein Formula (II) bas a structure of Formula (IIB), Formula (IIC), Formula (IID), Formula (IIE), or Formula (IIF):

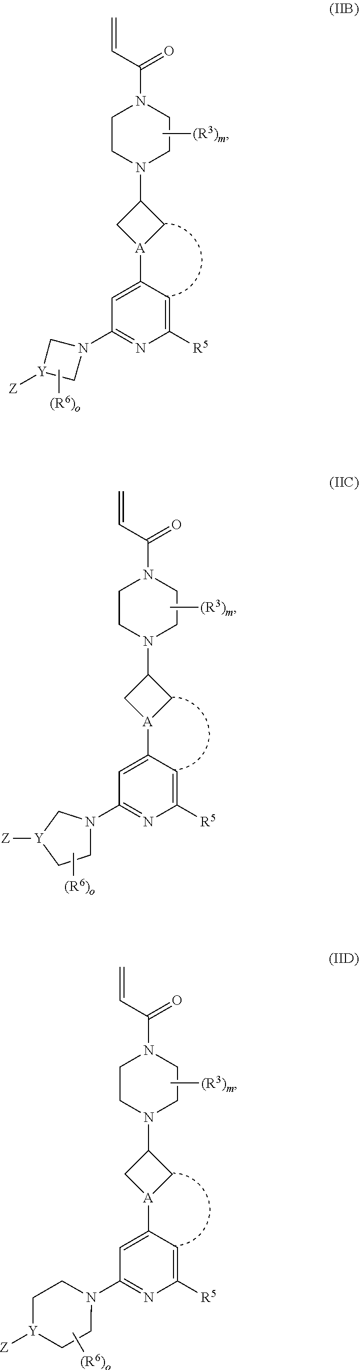

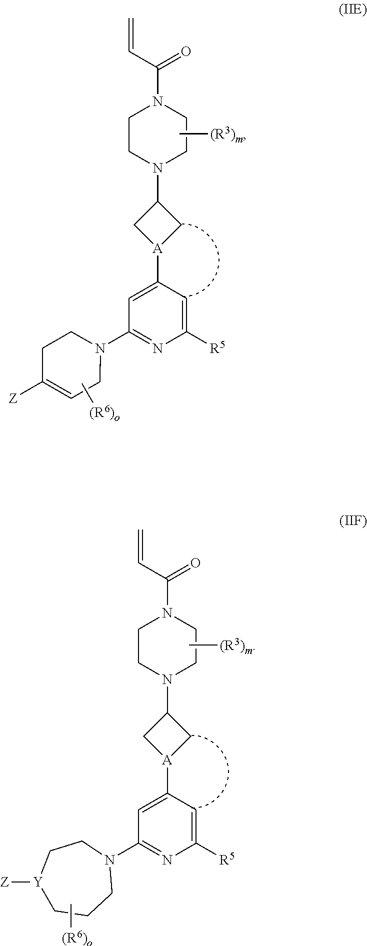

or a pharmaceutically acceptable salt of any of the foregoing.

Provided herein as Embodiment 315 is the compound of any one of Embodiments 58-314, wherein Formula (II) has a structure of Formula (II′):

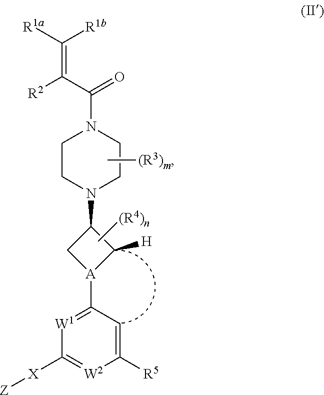

or a pharmaceutically acceptable salt thereof.

Provided herein as Embodiment 316 is the compound of Embodiment 58, wherein the compound is a compound listed in Table A, or a pharmaceutically acceptable salt thereof.

Provided herein as Embodiment 317 is the compound of Embodiment 316, wherein the compound is a compound listed in Table B, or a pharmaceutically acceptable salt thereof.

Provided herein as Embodiment 318 is the compound of Embodiment 58, wherein the compound is a compound listed in Table A′, or a pharmaceutically acceptable salt thereof.

Provided herein as Embodiment 319 is the compound of Embodiment 318, wherein the compound is a compound listed in Table B′ or a pharmaceutically acceptable salt thereof.

Provided herein as Embodiment 320 is a pharmaceutical composition comprising the compound or salt of any one of Embodiments 1-319 and a pharmaceutically acceptable excipient.

Provided herein as Embodiment 321 is a method of treating cancer in a subject in need of treatment, the method comprising administering to the subject a therapeutically effective amount of the compound or salt of any one of Embodiments 1-319, or the composition of Embodiment 320.

Provided herein as Embodiment 322 is the method of Embodiment 321, wherein one or more cells cancer express KRAS G12C mutant protein.

Provided herein as Embodiment 323 is the method of Embodiment 321 or 322, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, a solid tumor, or any combination of the foregoing.

Provided herein as Embodiment 324 is the method of Embodiment 323, wherein the cancer is non-small cell lung cancer, colorectal cancer, pancreatic cancer, appendiceal cancer, endometrial cancer, esophageal cancer, cancer of unknown primary, ampullary cancer, gastric cancer, small bowel cancer, sinonasal cancer, bile duct cancer, melanoma, a solid tumor, or any combination of the foregoing.

Provided herein as Embodiment 325 is the method of Embodiment 324, wherein the cancer is non-small cell lung cancer.

Provided herein as Embodiment 326 is the method of Embodiment 324, wherein the cancer is colorectal cancer.

Provided herein as Embodiment 327 is the method of Embodiment 324, wherein the cancer is pancreatic cancer.

Provided herein as Embodiment 328 is the method of Embodiment 324, wherein the cancer is solid tumor.

Provided herein as Embodiment 329 is the method according to any one of Embodiments 321-328, wherein the subject has a cancer that was determined to have one or more cancer cells expressing the KRAS G12C mutant protein prior to administration of the compound, salt, or pharmaceutical composition.

Provided herein as Embodiment 330 is the method according to any one of Embodiments 321-329, further comprising simultaneous, separate, or sequential administration of an effective amount of a second compound, wherein the second compound is an ATR inhibitor, Aurora kinase A inhibitor, AKT inhibitor, arginase inhibitor, CDK2 inhibitor, CDK4/6 inhibitor, ErbB family inhibitor, ERK inhibitor, FAK inhibitor, FGFR inhibitor, glutaminase inhibitor, IGF-IR inhibitor, KIF18A inhibitor, MAT2A inhibitor, MCL-1 inhibitor, MEK inhibitor, mTOR inhibitor, PARP inhibitor, PD-1 inhibitor, PD-L1 inhibitor, PI3K inhibitor, PRMT5 inhibitor, Raf kinase inhibitor, SHP2 inhibitor, SOS1 inhibitor, Sre kinase inhibitor, or one or more chemotherapeutic agents.

Provided herein as Embodiment 331 is the compound or salt of any one of Embodiments 1-319, or the composition of Embodiment 320 for use as a medicament.

Provided herein as Embodiment 332 is the compound or salt of any one of Embodiments 1-319, or the composition of Embodiment 320 for use in treating cancer.

Provided herein as Embodiment 333 is the use of the compound or salt of any one of Embodiments 1-319, or the pharmaceutical composition of Embodiment 320, for the manufacture of a medicament for the treatment of cancer.

Provided herein as Embodiment 334 is compound or salt of Embodiment 332 or the use of Embodiment 333, wherein one or more cancer cells express KRAS G12C mutant protein.

Provided herein as Embodiment 335 is the compound, salt, or use of any one of Embodiments 332-334, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, a solid tumor, or any combination of the foregoing.

Provided herein as Embodiment 336 is the use of Embodiment 335, wherein the cancer is non-small cell lung cancer, colorectal cancer, pancreatic cancer, appendiceal cancer, endometrial cancer, esophageal cancer, cancer of unknown primary, ampullary cancer, gastric cancer, small bowel cancer, sinonasal cancer, bile duct cancer, melanoma, a solid tumor, or any combination of the foregoing.

Provided herein as Embodiment 337 is the use of Embodiment 336, wherein the cancer is non-small cell lung cancer.

Provided herein as Embodiment 338 is the use of Embodiment 336, wherein the cancer is colorectal cancer.

Provided herein as Embodiment 339 is the use of Embodiment 336, wherein the cancer is pancreatic cancer.

Provided herein as Embodiment 340 is the use of Embodiment 336, wherein the cancer is solid tumor.

Provided herein as Embodiment 341 is the use of any one of Embodiments 332-340, wherein the cancer was determined to have one or more cancer cells expressing the KRAS G12C mutant protein prior to administration of the compound, salt, or pharmaceutical composition.

Provided herein as Embodiment 342 is a compound of Formula (Int-AA):

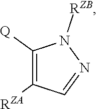

### Formula (Int-AB):

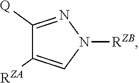

### Formula (Int-AC):

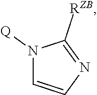

### Formula (Int-AD):

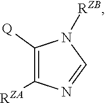

### Formula (Int-AE):

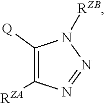

### Formula (Int-AF):

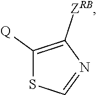

### Formula (Int-AG):

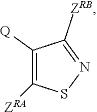

### Formula (Int-AH):

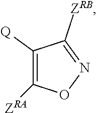

### Formula (Int-AI):

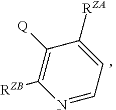

### or Formula (Int-AJ):

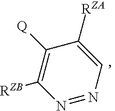

or a pharmaceutically acceptable salt thereof, wherein: Q is F, Cl, Br, I, or an organoborane; and each of R^{ZA} and R^{ZB} independently is halo, CN, C₁₋₆alkyl, C₁₋₆haloalkyl, C₂₋₆alkenyl, C₂₋₆haloalkenyl, C₀₋₆alkylene-OH, C₀₋₆alkylene-C₁₋₃alkoxy, C₀₋₆alkylene-N(R^{N1})₂, C₀₋₂alkylene-C₃₋₆cycloalkyl, C₀₋₂alkylene-heterocycloalkyl having 3-6 total ring atoms and 1-3 heteroatoms selected from N, O, and S, or C₀₋₂alkylene-phenyl; wherein each of the C₁₋₆alkyl, C₂₋₃alkenyl, C₀₋₃alkylene-C₁₋₃alkoxy, C₃₋₇cycloalkyl, heterocycloalkyl, and phenyl substituents independently is optionally substituted with 1 or more further substituents, and each RN′ independently is H or C₁₋₃alkyl.

Provided herein as Embodiment 343 is the compound or salt of Embodiment 342, wherein R^{ZA} is CH₃; and R^{ZB} is CH₃, C(CH₃)₂CH₂OH, CH₂CH₂OCH₃, CH₂CH₂OCD₃, CH(CH)OCH₃,

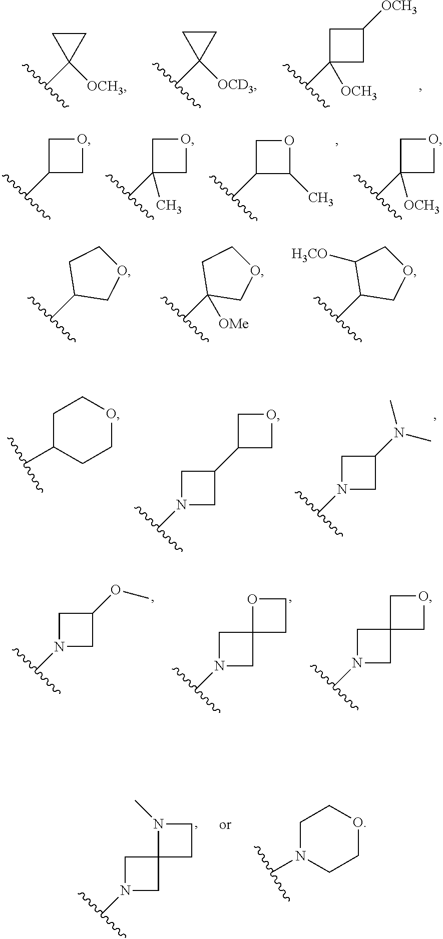

Provided herein as Embodiment 344 is the compound or salt of Embodiment 342, wherein the compound is a compound listed in Table INT-A or INT-A′, or a pharmaceutically acceptable salt thereof.

Provided herein as Embodiment 345 is a compound of Formula (Int-B);

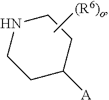

a nitrogen-protected analog thereof, or a pharmaceutically acceptable salt of any of the foregoing, wherein: A is

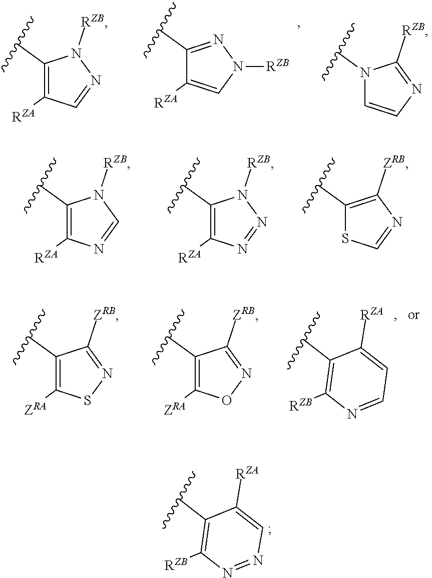

o is 0, 1, 2, 3, or 4; each R⁶ independently is halo, CN, C₁₋₃alkyl, C₂₋₃alkenyl, C₁₋₃haloalkyl, C₀₋₆alkylene-OH, C₀₋₃alkylene-C₁₋₃alkoxy, deuterated C₀₋₃alkylene-C₁₋₃alkoxy, or C₁₋₄alkylene-N(R^{N1})₂; or two geminal R⁶, together with the atom to which they are attached, form oxo, ═CH₂, spiro-C₃₋₇cycloalkyl, spiro-C₄₋₇cycloalkenyl, spiro-heterocycloalkyl having 4-7 total ring atoms and 1 or 2 heteroatoms selected from N, O and S, or spiro-heterocycloalkenyl having 4-7 total ring atoms and 1 or 2 heteroatoms selected from N, O and S; or two vicinal R⁶, together with the atoms to which they are attached, form fused-C₃₋₇cycloalkyl, fused-C₄₋₇cycloalkenyl, fused-heterocycloalkyl having 4-7 total ring atoms and 1-3 heteroatoms selected from N, O, and S, or fused-heterocycloalkenyl having 4-7 total ring atoms and 1 or 2 heteroatoms selected from N, O, and S; or two non-neighboring Rejoin together to form a C₁₋₃alkylene bridge or a C₁₋₃ether bridge; or Y and a vicinal R⁶, together with the atoms to which they are attached, form fused-C₃₋₇cycloalkyl, fused-C₄₋₇cycloalkenyl, fused-heterocycloalkyl having 3-7 total ring atoms and 1-3 heteroatoms selected from N, O, and S, or fused-heterocycloalkenyl having 4-7 total ring atoms and 1 or 2 heteroatoms selected from N, O, and S; wherein the cycloalkyl, cycloalkenyl, heterocycloalkyl or heterocycloalkenyl of any of the foregoing is unsubstituted or substituted with 1 or more substituents; and each of R^{ZA} and R^{ZB} independently is halo, CN, C₁₋₆alkyl, C₁₋₆haloalkyl, C₂₋₃alkenyl, C₂₋₆ haloalkenyl, C₀₋₆alkylene-OH, C₀₋₃alkylene-C₁₋₃alkoxy, C₀₋₃alkylene-N(R^{N1})₂, C₀₋₂alkylene-C₃₋₆cycloalkyl, C₀₋₂alkylene-heterocycloalkyl having 3-6 total ring atoms and 1-3 heteroatoms selected from N, O, and S, or C₀₋₂alkylene-phenyl; wherein each of the C₁₋₃alkyl, C₂₋₆alkenyl, C₀₋₆alkylene-C₁₋₃alkoxy, C₃₋₇cycloalkyl, heterocycloalkyl, and phenyl substituents independently is optionally substituted with 1 or more further substituents, and each R^{N1} independently is H or C₁₋₃alkyl.

Provided herein as Embodiment 346 is the compound or salt of Embodiment 345, wherein o is 0 or 1; R⁶ is CH; R^{ZA} is CH₃; and R^{ZB} is CH₃, C(CH₃)₂CH₂OH, CH₂CH₂OCH₃, CH₂CH₂OCD₃, CH(CH₃)OCH₃.

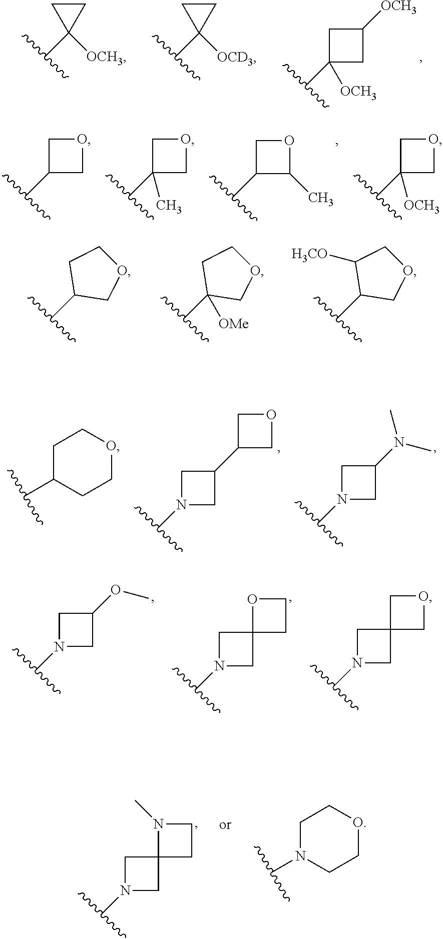

Provided herein as Embodiment 347 is the compound of salt of Embodiment 345, wherein the compound is a compound listed in Table INT-B, or a pharmaceutically acceptable salt thereof.

Provided herein as Embodiment 348 is a compound of Formula (Int-C):

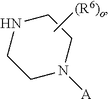

a nitrogen-protected analog thereof, or a pharmaceutically acceptable salt of any of the foregoing, wherein: A is

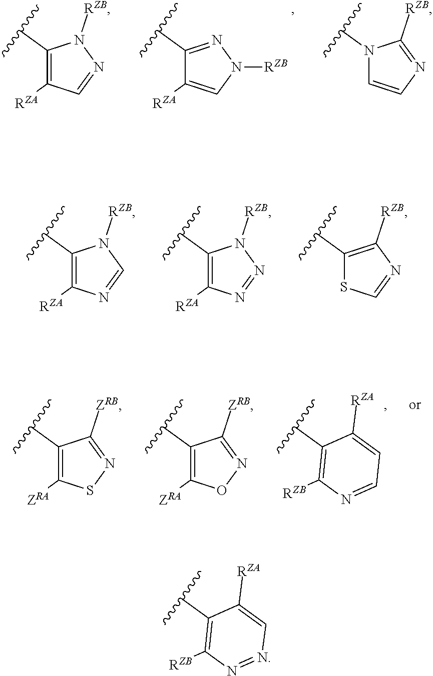

### A is

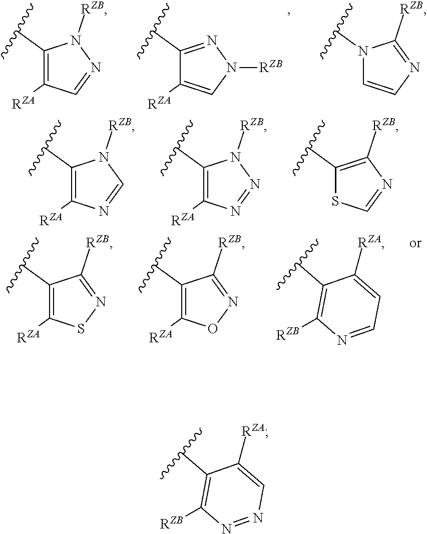

o is 0, 1, 2, 3, or 4; each R⁶ independently is halo, CN, C₁₋₃alkyl, C₂₋₃alkenyl, C₁₋₃haloalkyl, C₀₋₃alkylene-OH, C₀₋₃alkylene-C₁₋₃alkoxy, deuterated C₀₋₆alkylene-C₁₋₃alkoxy, or C₁₋₄alkylene-N(R^{N1})₂; or two geminal R⁶, together with the atom to which they are attached, form oxo, ═CH₂, spiro-C₃₋₇cycloalkyl, spiro-C₄₋₇cycloalkenyl, spiro-heterocycloalkyl having 4-7 total ring atoms and 1 or 2 heteroatoms selected from N, O and S, or spiro-heterocycloalkenyl having 4-7 total ring atoms and 1 or 2 heteroatoms selected from N, O and S; or two vicinal R⁶, together with the atoms to which they are attached, form fused-C₃₋₇cycloalkyl, fused-C₄₋₇cycloalkenyl, fused-heterocycloalkyl having 4-7 total ring atoms and 1-3 heteroatoms selected from N, O, and S, or fused-heterocycloalkenyl having 4-7 total ring atoms and 1 or 2 heteroatoms selected from N, O, and S; or two non-neighboring R⁶ join together to form a C₁₋₂alkylene bridge or a C₁₋₃ether bridge; or Y and a vicinal R⁶, together with the atoms to which they are attached, form fused-C₃₋₇cycloalkyl, fused-C₄₋₇cycloalkenyl, fused-heterocycloalkyl having 3-7 total ring atoms and 1-3 heteroatoms selected from N, O, and S, or fused-heterocycloalkenyl having 4-7 total ring atoms and 1 or 2 heteroatoms selected from N, O, and S; wherein the cycloalkyl, cycloalkenyl, heterocycloalkyl or heterocycloalkenyl of any of the foregoing is unsubstituted or substituted with 1 or more substituents; and each of R^{ZA} and R^{ZB} independently is halo, CN, C₁₋₆alkyl, C₁₋₆haloalkyl, C₂₋₆alkenyl, C₂₋₆ haloalkenyl, C₀₋₆alkylene-OH, C₀₋₆alkylene-C₁₋₃alkoxy, C₀₋₃alkylene-N(R^{N1})₂, C₀₋₂alkylene-C₃₋₆cycloalkyl, C₀₋₂alkylene-heterocycloalkyl having 3-6 total ring atoms and 1-3 heteroatoms selected from N, O, and S, or C₀₋₂alkylene-phenyl; wherein each of the C₁₋₆alkyl, C₂₋₃alkenyl, C₀₋₆alkylene-C₁₋₃alkoxy, C₃₋₇cycloalkyl, heterocycloalkyl, and phenyl substituents independently is optionally substituted with 1 or more further substituents, and each R^{N1} independently is H or C₁₋₃alkyl.

Provided herein as Embodiment 349 is the compound or salt of Embodiment 348, wherein o is 0 or 1; R⁶ is CH₃; R^{ZA} is CH₃; and R^{ZB} is CH₃, C(CH₃)₂CH₂OH, CH₂CH₂OCH₃, CH₂CH₂OCD₃, CH(CH₃)OCH₃,

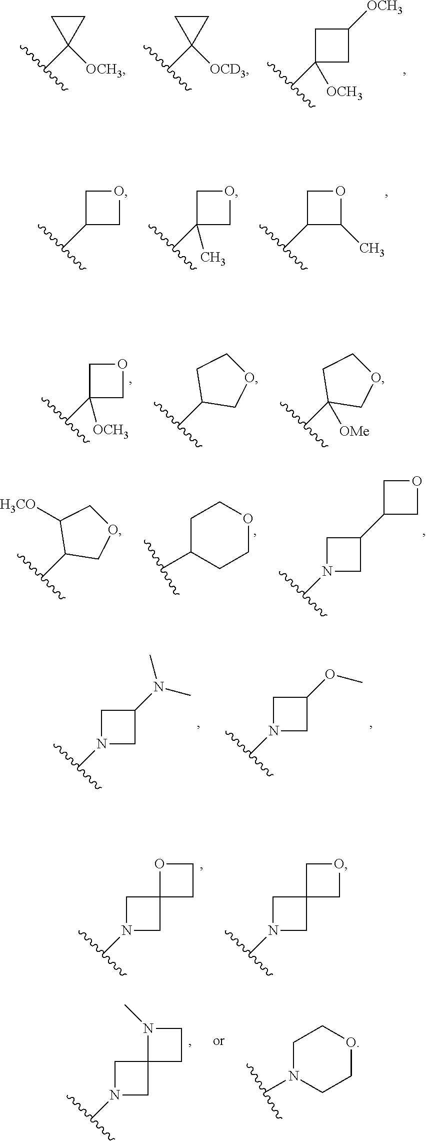

Provided herein as Embodiment 350 is the compound or salt of Embodiment 348, wherein the compound is a compound listed in Table INT-C, or a pharmaceutically acceptable salt thereof.

Provided herein as Embodiment 351 is a compound of Formula (Int-D):

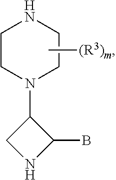

a nitrogen-protected analog thereof, or a pharmaceutically acceptable salt of any of the foregoing, wherein: m is 0, 1, 2, 3, or 4; each R³ independently is C₁₋₃alkyl, C₁₋₃haloalkyl,

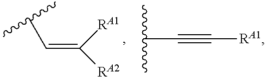

C₀₋₃alkyleneCN, C₀₋₆alkyleneOH, or C₀₋₆alkylene-C₁₋₃alkoxy; or two geminal R³, together with the atom to which they are attached, form oxo, spiro-C₃₋₇cycloalkyl, spiro-C₄₋₇cycloalkenyl, spiro-heterocycloalkyl having 3-7 total ring atoms and 1 or 2 heteroatoms selected from N, O, and S, or spiro-heterocycloalkenyl having 4-7 total ring atoms and 1 or 2 heteroatoms selected from N, O, and S; or two vicinal R³, together with the atoms to which they are attached, form fused-C₃₋₇cycloalkyl, fused-C₄₋₇cycloalkenyl, fused-heterocycloalkyl having 3-7 total ring atoms and 1 or 2 heteroatoms selected from N, O, and S, or fused-heterocycloalkenyl having 4-7 total ring atoms and 1 or 2 heteroatoms selected from N, O, and S; and B is C₁₋₃alkylene-CH═CH₂ or C₁₋₃alkyleneOH.

Provided herein as Embodiment 352 is the compound of salt of Embodiment 351, wherein m is 0 or 1; R³ is CH₃; and B is CH₂CH═CH, or CH₂CH₂OH

Provided herein as Embodiment 353 is the compound or salt of Embodiment 351, wherein the compound is a compound listed in Table INT-D, or a pharmaceutically acceptable salt thereof.

Provided herein as Embodiment 354 is a compound of Formula (Int-E):

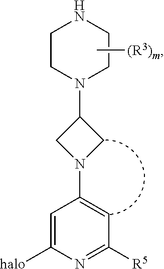

wherein: m is 0, 1, 2, 3 or 4; halo is F, Cl, Br, or I; [symbol] is C₂₋₆alkylene, C₂₋₆alkenylene, heteroalkylene having 2-6 total atoms and 1-3 heteroatoms selected from N, O, and S, or heteroalkenylene having 3-6 total atoms and 1 or 2 heteroatoms selected from N, O, and S, wherein [symbol] is unsubstituted or substituted with 1 or more substituents; each R³ independently is C₁₋₃alkyl, C₁₋₃haloalkyl,

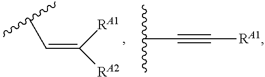

C₀₋₃alkyleneCN, C₀₋₃alkyleneOH, or C₀₋₆alkylene-C₁₋₃alkoxy; or two geminal R³, together with the atom to which they are attached, form oxo, spiro-C₃₋₇cycloalkyl, spiro-C₃₋₇cycloalkenyl, spiro-heterocycloalkyl having 3-7 total ring atoms and 1 or 2 heteroatoms selected from N, O, and S, or spiro-heterocycloalkenyl having 4-7 total ring atoms and 1 or 2 heteroatoms selected from N, O, and S; or two vicinal R³, together with the atoms to which they are attached, form fused-C₃₋₇cycloalkyl, fused-C₄₋₇cycloalkenyl, fused-heterocycloalkyl having 3-7 total ring atoms and 1 or 2 heteroatoms selected from N, O, and S, or fused-heterocycloalkenyl having 4-7 total ring atoms and 1 or 2 heteroatoms selected from N, O, and S; and R⁵ is halo, C₁₋₃haloalkyl, C₁₋₄alkyl, C₂₋₃alkenyl, C₂₋₃alkynyl, C₁₋₃alkoxy, C₁₋₃thioalkyl, C₃₋₇cycloalkyl, C₅₋₇cycloalkenyl, heterocycloalkyl having 3-7 total ring atoms and 1-3 heteroatoms selected from N, O, and S, or heterocycloalkenyl having 5-7 total ring atoms and 1-3 heteroatoms selected from N, O, and S, wherein each of the foregoing is independently unsubstituted or substituted with 1 or more substituents.

Provided herein as Embodiment 355 is the compound or salt of Embodiment 354, wherein halo is Cl, m is 0 or 1; R⁵ is CH₃; R⁵ is CHF₂ or CF₃; and [symbol] is

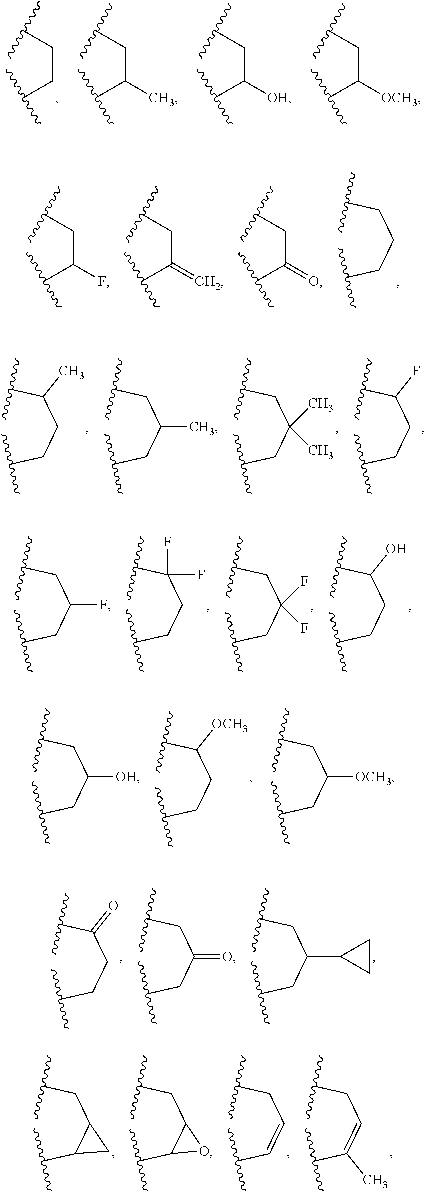

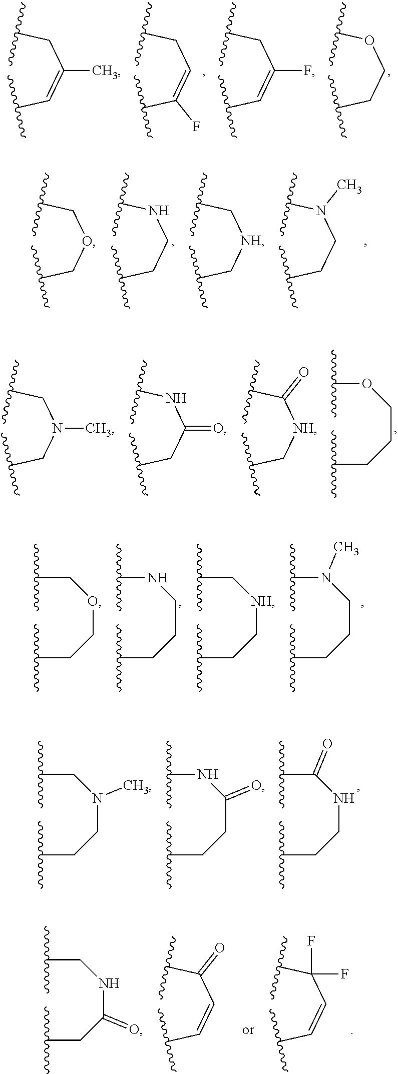

Provided herein as Embodiment 356 is the compound or salt of Embodiment 354, wherein the compound is a compound listed in Table INT-E, or a pharmaceutically acceptable salt thereof.

Provided herein as Embodiment 357 is a compound listed in Table INT-F or Table INT, or a pharmaceutically acceptable salt thereof.

Provided herein as Embodiment 358 is a process for preparing the compound or salt of any one of Embodiments 58-319, comprising converting a compound or salt of any one of Embodiments 342-357 into a compound or salt of any one of Embodiments 58-319,

The following examples are given for the purpose of illustrating various embodiments of the disclosure and are not meant to limit the present disclosure in any fashion. One skilled in the art will appreciate readily that the present disclosure is well-adapted to carry out the objects and obtain the ends and advantages mentioned, as well as those objects, ends, and advantages inherent herein. Changes therein and other uses which are encompassed within the spirit of the disclosure as defined by the scope of the claims will occur to those skilled in the art.

### EXAMPLES

This section provides specific examples of compounds of Formula (I) and methods of making the same.

### List of Abbreviations

| | |
|---|---|
| Ac | acetyl |
| ACN or MeCN | acetonitrile |
| AcOH | acetic acid |
| anh or anhy | anhydride |
| aq or aq. | aqueous |
| Bn | benzyl |
| B₂pin₂ | 4,4,4′,4′,5,5,5′,5′-octamethyl-2,2′-bi(1,3,2-dioxaborolane) |
| BOC or Boc | tert-butyloxycarbonyl |
| Bu | butyl |
| Cbz | benzyloxycarbonyl |
| CbzCl | benzyl chloroformate |
| CV | column volume |
| Cy | cyclohexyl |
| DAST | diethylaminosulfur trifluoride |
| DCE | dichloroethane |
| DCM | dichloromethane |
| DEA | diethylamine |
| DMA | N,N-dimethylacetamide |
| DMAP | 4-dimethylaminopyridine |
| DMF | N,N-dimethylformamide |
| DMSO | dimethyl sulfoxide |
| DPPA | diphenyl phosphoryl azide |
| DPPF or dppf | bis(diphenylphosphino)ferrocene |
| eq or eq. or equiv. | equivalent |
| ESI or ES | electrospray ionization |
| Et | ethyl |
| EtOAc | ethyl acetate |
| EtOH | ethanol |
| g or gr | gram(s) |
| h | hour(s) |
| HBpin | 4,4,5,5-tetramethyl-1,3,2-dioxaborolane |
| HPLC | high pressure liquid chromatography |
| iPr | iso-propyl |
| iPrOH or IPA | iso-propanol |
| iPAm or IPAm | iso-propylamine |
| iPr₂NET or DIPEA | N-ethyl diisopropylamine (Hünig's base) |
| KHMDS | potassium hexamethyldisilazide |
| KOAc | potassium acetate |
| LAH | lithium aluminum hydride |
| LC MS, LCMS, LC-MS | liquid chromatography mass spectroscopy |
| or LC/MS | |
| LDA | lithium diisopropylamide |
| LHMDS or LiHMDS | lithium hexamethyldisilazide |
| m/z | mass divided by charge |
| mCPBA | meta-chloroperoxybenzoic acid |
| Me | methyl |
| MeI | iodomethane |
| MeOH | methanol |
| mg | milligrams |
| min | minutes |
| mL | milliliters |
| Mn(dpm)₃ | tris(2,2,6,6-tetramethyl-3,5-heptanedionato)manganese(III) |
| MS | mass spectra |
| MsCl | methanesulfonyl chloride |
| MTBE | methyl tert-butyl ether |
| NaHMDS | sodium hexamethylsilazide |
| NBS | N-bromosuccinimide |
| NIS | N-iodosuccinimide |
| NMP | N-methyl-2-pyrrolidinone |
| NMR | nuclear magnetic resonance |
| Pd(dppf)Cl₂•DCM, | [1,1′-bis(diphenylphosphino)ferrocene]dichloropalladium(II), |
| Pd(dppf)Cl₂ | complex with DCM |
| Pd(dtbpf)Cl2 | [1,1′-bis(di-tert-butylphosphino)ferrocene]dichloropalladium(II) |
| Pd(PPh₃)₄ | tetrakis(triphenylphosphine)palladium(0) |
| PEPPSI-IPr | [1,3-bis(2,6-diisopropylphenyl)imidazol-2-ylidene](3- |
| | chloropyridyl)palladium(II) dichloride |
| pet. ether | petroleum ether |
| Ph | phenyl |
| PhMe | toluene |
| Pin | pinacolato |
| PMB | 4-methoxybenzyl |
| PTSA or pTsOH | p-toluenesulfonic acid |
| RP-HPLC | reverse phase high pressure liquid chromatography |
| RT or rt or r.t. | room temperature |
| RuPhos | 2-dicyclohexylphosphino-2′,6′-diisopropoxy-1,1′-biphenyl. |
| | dicyclohexyl(2′,6′-diisopropoxy-[1,1′-biphenyl]-2-yl)phosphine. |
| RuPhos Pd G1 MTBE | chloro-(2-dicyclohexylphosphino-2′,6′-diisopropoxy-1,1′- |
| | biphenyl)[2-(2-aminoethyl)phenyl]palladium(II) - methyl-t-butyl |
| | ether adduct |
| sat. or satd. | saturated |
| SFC | supercritical fluid chromatography |
| SPhos | 2-dicyclohexylphosphino-2′,6′-dimethoxybiphenyl |
| SPhos Pd G3 | (2-dicyclohexylphosphino-2′,6′-dimethoxybiphenyl) [2-(2′-amino- |
| | 1,1′-bipbenyl)]palladium(II) methanesulfonate |
| TBAF | tetra-n-butylammonium fluoride |
| tBu | tert-butyl |
| TBHP | tert-butyl hydroperoxide |
| TEA or Et₃N | triethylamine |
| temp | temperature |
| Tf | trifluoromethylsulfonyl |
| TFA | trifluoroacetic acid |
| THF | tetrahydrofuran |
| TLC | thin layer chromatography |
| TsCl | 4-toluenesulfonyl chloride |
| UV | ultraviolet |
| XPhos | 2-dicyclohexylphosphino-2′,4′,6′-triisopropyl-1,1′-biphenyl |
| XPhos Pd G2 | chloro(2-dicyclohexylphosphino-2′,4′,6′-triisopropyl-1,1′- |
| | biphenyl)[2-(2′-amino-1,1′-biphenyl)]palladium(II) |

Provided in this section are descriptions of the general analytical and purification methods used to prepare the specific examples provided herein.

Chromatography: Unless otherwise indicated, product-containing residues were purified by passing the material or concentrate through silica gel using a Biotage Sfar HCD, and eluting the product off the column with a solvent gradient as indicated.

Preparative HPLC Method: Where indicated, the compounds described herein were purified via reverse phase HPLC using Waters FractionLynx or Gilson semi-preparative HPLC-MS system utilizing one of the following two HPLC columns: (a) Phenomenex Gemini column (5 micron, C18, 150×30 mm) or (b) Waters X-select CSH column (5 micron, C18, 100×30 mm). A typical run through the instrument included: eluting at 45 mL/min with a linear gradient of 10% (v/v) to 100% MeCN (0.1% v/v formic acid) in H₂O (0.1% formic acid) over 10 min.

Proton NMR Spectra: Unless otherwise indicated, all ¹H NMR spectra were collected on a Bruker NMR instrument at 300, 400 or 500 MHz. All observed protons are reported as parts-per-million (ppm) downfield from tetramethylsilane (TMS) using the internal solvent peak as reference. Some 1H signals may be missing due to exchange with D from CD₃OD, or due to signal suppression.

Fluorine-19 NMR Spectra: Unless otherwise indicated, all ¹⁹F NMR spectra were collected on a Bruker NMR instrument at 300 or 400 MHz.

Mass Spectra (MS): Unless otherwise indicated, all mass spectral data for starting materials, intermediates and/or exemplary compounds are reported as mass/charge (m/z), having an (M+H)⁺ molecular ion. The molecular ion reported was obtained by electrospray detection method (commonly referred to as an ESI MS) utilizing a Waters Acquity UPLC/MS system. Compounds having an isotopic atom, such as bromine and the like, are generally reported according to the detected isotopic pattern, as appreciated by those skilled in the art.

### Section 1: Synthesis of Intermediates

Provided in this section is the synthesis of various intermediates used to prepare compounds of Formula (I). All starting materials are either commercially available, unless otherwise noted, or known in the art and may be synthesized by employing known procedures using ordinary skill.

### Intermediate A1—5-Iodo-1-(2-methoxyethyl)-4-methyl-1H-pyrazole

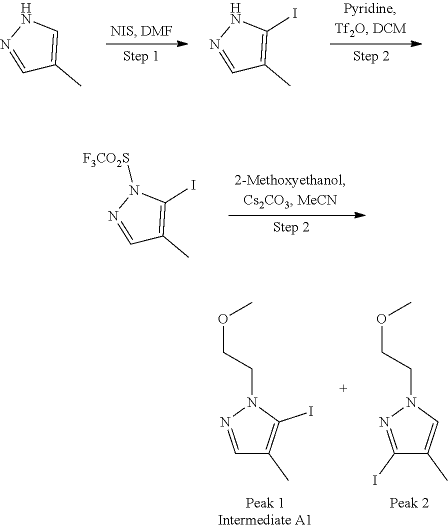

Step 1. 5-Iodo-4-methyl-1H-pyrazole. To a solution of 4-methyl-1H-pyrazole (500 g, 6.1 mol, Arbor) in DMF (5000 mL) at rt was added NIS (1370 g, 6.1 mol, Spectrochem) and the mixture was heated at 65° C. for 1 h. The reaction mixture was quenched with crushed ice (10 L) and extracted with tert-butyl methyl ether (3×5 L). The organic extract was washed with Satd. aq. sodium thiosulphate (5 L), brine (5 L), dried over Na₂SO₄, filtered, and concentrated. The crude residue was purified by column chromatography (10 to 20% EtOAc:hexanes) to give 5-iodo-4-methyl-1H-pyrazole, m/z (ESI): 209.1 (M+H)⁺. ¹H NMR (400 MHz, DMSO-d₆) δ (ppm) 12.96 (s, 1H), 7.50 (s, 1H), 1.92 (s, 3H).

Stop 2. 5-Jodo-4-methyl-1-((trifluoromethyl) sulfonyl)-1H-pyrazole. To a mixture of 5-iodo-4-methyl-1H-pyrazole (158 g, 760 mmol) and pyridine (92 mL, 1139 mmol, Sonia Industries) in DCM (1580 mL) at 0° C. was slowly added trifluoromethanesulfonic anhydride (154 mL, 912 mmol, Avra Synthesis), and the mixture was stirred at rt for 30 min. The reaction was quenched by addition into ice-cold water (3000 mL) and extracted with DCM (2 L). The organic extract was washed with brine (2 L), dried over Na₂SO₄, filtered, and concentrated to give 5-iodo-4-methyl-1-((trifluoromethyl) sulfonyl)-1H-pyrazole, m/z (ESI): No ionization, ¹H NMR (400 MHz, DMSO-d₆) δ (ppm) 8.44 (s, 1H), 2.02 (s, 3H).

Step 3. 5-Iodo-1-(2-methoxyethyl)-4-methyl-1H-pyrazole, Intermediate A1. To a mixture of 5-iodo-4-methyl-1-((trifluoromethyl) sulfonyl)-1H-pyrazole (360 g, 1059 mmol) and Cs₂CO₃ (517 g, 1588 mmol, Avra Synthesis) in MeCN (3600 mL) at 0° C. was added 2-methoxyethan-1-ol (100 mL, 1270 mmol, TCI) dropwise. The reaction mixture was stirred at rt for 1 h. The reaction mixture was quenched with water (5 L) and extracted with EtOAc (3 L). The organic extract was washed with brine (3000 mL), dried over Na₂SO₄, filtered, and concentrated. The crude residue was purified by column chromatography (10 to 20% EtOAc:hexanes) to give a mixture of 5-iodo-1-(2-methoxyethyl)-4-methyl-1H-pyrazole and 3-iodo-1-(2-methoxyethyl)-4-methyl-1H-pyrazole.

The regioisomers were separated by SFC [Chiral Pak IC (150×50 mm, 5μ) with a mobile phase of 90% CO₂ and 10% MeOH using a flow rate of 150 mL/min] to give a 1^{st} eluting isomer and a 2^{nd} eluting isomer. The 1st eluting isomer was assigned as 5-iodo-1-(2-methoxyethyl)-4-methyl-1H-pyrazole (Intermediate A1), and the 2^{nd} eluting isomer was assigned as 3-iodo-1-(2-methoxyethyl)-4-methyl-1H-pyrazole. 1st eluting isomer: ¹H NMR (400 MHz, DMSO-d₆) δ (ppm) 7.42 (s, 1H), 4.25 (t, 2H, J=5.7 Hz), 3.66 (1, 2H, J=5.7 Hz), 3.21 (s, 3H), 1.95 (s, 3H) m/z (ESI): 267.1 (M+H)⁺. 2^{nd} eluting isomer: ¹H NMR (400 MHz, DMSO-d₆) δ (ppm) 7.47 (s, 1H), 4.20 (t, 2H, J=5.3 Hz,), 3.63 (t, 2H, J=5.3 Hz,), 3.22 (s, 3H), 1.89 (s, 3H).

Intermediates in Table 1-1 were prepared following the procedure described for Intermediate A1, using appropriate starting materials. All starting materials are commercially available or are described above.

TABLE 1-1

| | Chemical Structure & Name | LCMS: (ESI + ve ion) m/z; NMR | Comments |
|---|---|---|---|
| A2 | 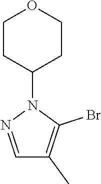 | m/z (ESI): 246.9 (M + H)⁺. ¹H NMR (DMSO-d₆, 400 MHz): δ (ppm) 7.47 (s, 1H), 4.53-4.41 (m, 1H), 4.00-3.92 (m, 2H), 3.54- 3.43 (m, 2H), 2.11-1.98 (m, 2H), 2.00-1.92 (m, 3H), 1.79 (m, 2H). | Step 1. NBS was used |
| | 5-bromo-4-methyl-1- | | |
| | (tetrahydro-2H-pyran-4-yl)- | | |
| | 1H-pyrazole | | |
| A3 | 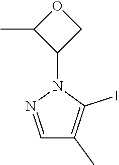 | m/z (ESI): 279.6 (M + H)⁺. ¹H NMR (CDCl₃, 400 MHz): δ ppm 7.54 (s, 1 H), 5.30 (m, 1 H), 5.05- 5.16 (m, 2 H), 4.74-4.85 (m, 1 H), 2.05 (s, 3 H), 1.57 (d, J = 6.1 Hz, 3 H). | |
| | 5-iodo-4-methyl-1-(2- | | |
| | methyloxetan-3-yl)-1H- | | |
| | pyrazole | | |

### Intermediate A4-5-Bromo-4-methyl-1-(oxetan-3-yl)-1H-pyrazole

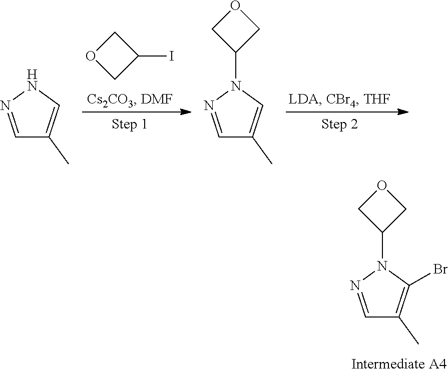

Step 1. 4-Methyl-1-(oxetan-3-yl)-1H-pyrazole. To a mixture of 4-methyl-1H-pyrazole (50.0 g, 609 mmol, Combi-Blocks, Inc.) and Cs₂CO³ (397 g, 1218 mmol, Chempure) in DMF (750 mL) was added 3-iodooxetane (168 g, 913 mmol, Oakwood), and the mixture was heated at 80° C. for 16 h. The mixture was cooled to rt, quenched with water (3000 mL), and extracted with EtOAc (1000 mL). The organic extract was washed with brine, concentrated, and then purified by chromatography (0 to 20% EtOAc:hexanes) to give 4-methyl-1-(oxetan-3-yl)-1H-pyrazole, m/z (ESI): 139.2 (M+H)⁺. ¹H NMR (400 MHz, DMSO-d₆) δ (ppm) 7.64 (s, 1H), 7.37 (s, 1H), 5.4-5.5 (m, 1H), 4.7-4.9 (m, 4H), 2.02 (S. 3H).

Step 2. 5-Bromo-4-methyl-1-(oxetan-3-yl)-1H-pyrazole, Intermediate A4. LDA (2 M solution in THF, 326 mL, 651 mmol, Sigma-Aldrich) was added to a solution of 4-methyl-1-(oxetan-3-yl)-1H-pyrazole (60 g, 434 mmol) in THF (750 mL) at −78° C., and the mixture was stirred for 30 min. Carbon tetrabromide (216 g. 651 mmol, TCI) in THF (500 mL) was added dropwise over 1 h at −78° C., and the mixture was stirred for another 1 h. The reaction mixture was quenched with satd. aq. NH₄Cl (1500 mL) and extracted with EtOAc (600 mL). The organic extract was dried over Na₂SO₄ concentrated, and then purified by silica gel chromatography (0 to 15% EtOAc:hexanes) to give 5-bromo-4-methyl-1-(oxetan-3-yl)-1H-pyrazole, m/z (ESI): 217.0 and 218.9 (M+H)⁺. ¹H NMR (400 MHz, DMSO-d₆) δ (ppm) 7.59 (s, 1H), 5.61 (p, J=6.9 Hz, 1H), 4.90 (d, J=6.9 Hz, 4H), 1.97 (s, 3H).

### Intermediate A5-3-Chloro-4-methyl-2-(3-(oxetan-3-yl)azetidin-1-yl)pyridine

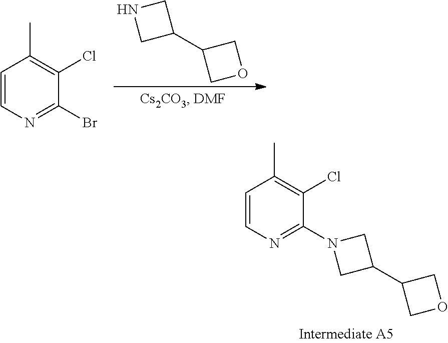

A mixture of 2-bromo-3-chloro-4-methylpyridine (0.718 g, 3.48 mmol, Combi-Blocks Inc.), bis(3-(oxetan-3-yl)azetidine) oxalic acid (1.0 g, 3.16 mmol, Enamine) and cesium carbonate (3.09 g, 9.48 mmol, Combi-Blocks Inc.) in DMF (20 mL) was stirred at 98° C. for 17 h. The reaction was diluted with EtOAc and filtered through celite. The filtrate was washed with water and the organic phase was dried over Na₂SO₄, filtered, concentrated, and chromatographed on silica gel using 0-30% EtOAc in heptane to afford 3-chloro-4-methyl-2-(3-(oxetan-3-yl)azetidin-1-yl)pyridine (0.253 g), m/z (ESI): 239.0 (M+H)⁺.

Intermediates in Table 1-2 were prepared following the procedure described for Intermediate A5, using appropriate starting materials. All starting materials are commercially available or are described above.

TABLE 1-2

| | Chemical Structure & Name | LCMS: (ESI + ve ion) m/z; NMR | Comments |
|---|---|---|---|
| A6 | 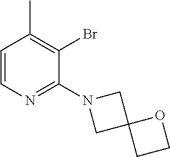 | m/z (ESI): 269.0 and 271.0 (M + H)⁺. | 3-bromo-2-chloro-4- methylpyridine was used |
| | 6-(3-bromo-4- | | |
| | methylpyridin-2-yl)-1-oxa- | | |
| | 6-azaspiro[3.3]heptane | | |
| A7 | 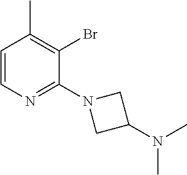 | m/z (ESI): 270.0 and 272.0 (M + H)⁺. | 3-bromo-2-chloro-4- methylpyridine was used |
| | 1-(3-bromo-4- | | |
| | methylpyridin-2-yl)-N,N- | | |
| | dimethylazetidin-3-amine | | |
| A8 | 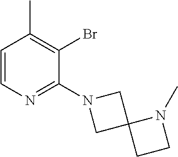 [SMILES: Cc1ccnc(N2CC3(CCN3C)C2)c1Br] | m/z (ESI): 282.0 and 284.0 (M + H)⁺. | 3-bromo-2-chloro-4- methylpyridine was used |
| | 6-(3-Bromo-4- | | |
| | methylpyridin-2-yl)-1- | | |
| | methyl-1,6- | | |
| | diazaspiro[3.3]heptane | | |
| A9 | 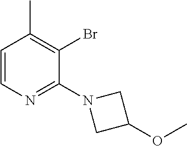 | m/z (ESI): 257.0 and 259.0 (M + H)⁺. | 3-bromo-2-chloro-4- methylpyridine was used |
| | 3-bromo-2-(3- | | |
| | methoxyazetidin-1-yl)-4- | | |
| | methylpyridine | | |

### Intermediate A10-(1-(1-Methoxycyclopropyl)-4-methyl-1H-pyrazol-5-yl) boronic acid

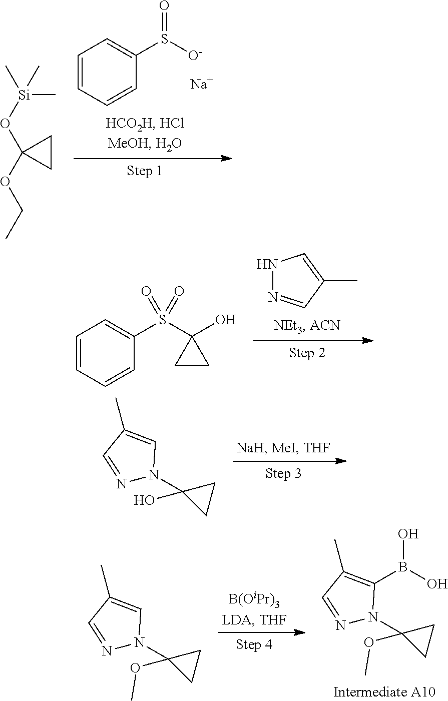

Step 1. 1-(Phenylsulfonyl)cyclopropan-1-ol. To a solution of (1-ethoxycyclopropoxy)trimethylsilane (10.00 mL, 57.4 mmol, Sigma-Aldrich Corporation) in MeOH (28.7 mL) was added conc. HCl (1 drop, Sigma-Aldrich Corporation) and the reaction mixture was stirred at rt for 10 min. To the reaction mixture was added water (57.3 mL), sodium benzenesulfinate (18.83 g, 115 mmol, Combi-Blocks Inc.), and formic acid 95-97% (21.6 mL, 574 mmol, Sigma-Aldrich Corporation), and the reaction mixture was stirred at rt over 2 d. The reaction mixture was diluted with water and extracted with DCM. The organic extract was washed with brine, dried over Na₂SO₄, filtered, and concentrated in vacuo to afford 1-(phenylsulfonyl)cyclopropan-1-ol that was used without further purification, m/z (ESI): 199.0 (M+Na)⁺.

Step 2. 1-(4-Methyl-1H-pyrazol-1-yl)cyclopropan-1-ol. 4-Methyl-1H-pyrazole (4.64 g, 56.5 mmol, Ambeed, Inc.) and 1-(phenylsulfonyl)cyclopropan-1-ol (11.8 g, 59.5 mmol) were mixed under N₂ atmosphere. To the reaction mixture was added ACN (119 mL) and Et₃N (8.4 mL, 59.5 mmol, Sigma-Aldrich Corporation), then the mixture was stirred at rt for 3 h. The organic phase was separated, and the aqueous phase was extracted with EtOAc (×2). The organic extracts were washed with brine, dried over Na₂SO₄, filtered, and concentrated in vacuo. The crude mixture was dissolved in MeCN, and the pH was adjusted to 9 with conc NH₄OH (aq) solution. Then, the aqueous phase was extracted with DCM (×2). The organic extracts were combined, washed with brine, dried over Na₂SO₄, filtered, and concentrated to afford 1-(4-methyl-1H-pyrazol-1-yl)cyclopropan-1-ol that was used in the next without further purification, m/z (BSI): 139.1 (M+H)⁺.

Step 3. 1-(1-Methoxycyclopropyl)-4-methyl-1H-pyrazole. To a 0° C. suspension of NaH (4.56 g, 114 mmol, TCI America, 60% in mineral oil) in THF (15 mL) was added a solution of 1-(4-methyl-1H-pyrazol-1-yl)cyclopropan-1-ol (7.88 g, 57.0 mmol) in THF (8 mL) dropwise, and the reaction mixture was stirred for 30 min. To the reaction mixture was added Mel (7.13 mL, 114 mmol, Sigma-Aldrich Corporation), and the reaction mixture was warmed to rt and stirred for 18 h. The reaction mixture was quenched by addition of water and was extracted with EtOAc. The organic extracts were washed with brine, dried over Na₂SO₄, filtered, and concentrated. The crude material was filtered over a plug of silica gel, eluted with EtOAc (2×250 mL) and concentrated in vacuo to afford 1-(1-methoxycyclopropyl)-4-methyl-1H-pyrazole, m/z (ESI): 153.2 (M+H)⁺.

Step 4. (1-(1-Methoxycyclopropyl)-4-methyl-1H-pyrazol-5-yl) boronic acid, Intermediate A10. To a solution of 1-(1-methoxycyclopropyl)-4-methyl-1H-pyrazole (2.12 g. 13.93 mmol) in THF (10.65 mL) at −78° C. was added a solution of LDA (2M in THF, 10.45 mL, 20.89 mmol, Sigma-Aldrich Corporation), and the reaction mixture was stirred for 30 min. To the reaction mixture at −78° C. was added triisopropyl borate (4.85 mL, 20.89 mmol, Sigma-Aldrich Corporation), and the reaction mixture was stirred for 1 h allowing to warmup to rt. The reaction mixture was quenched by the addition of sat. aq. NH₄Cl and extracted with EtOAc. The organic extracts were filtered and concentrated to provide the Intermediate A10, which was used without further purification, m/z (ESI): 197.2 (M+H)⁺

Intermediates in Table 1-3 were prepared following the procedure described for Intermediate A10, using appropriate starting materials. All starting materials are commercially available or are described above.

TABLE 1-3

| | Chemical Structure & Name | LCMS: (ESI + ve ion) m/z; NMR | Comments |
|---|---|---|---|
| A11 | 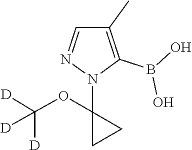 [SMILES: [2H]C([2H])([2H])OC1(n2ncc(C)c2B(O)O)CC1] | m/z (ESI): 200.2 (M + H)⁺. | |
| | (1-(1-(methoxy- | | |
| | d₃)cyclopropyl)-4-methyl- | | |
| | 1H-pyrazol-5-yl)boronic | | |
| | acid | | |

### Intermediate A12-(S)-5-Iodo-4-methyl-1-(tetrahydrofuran-3-yl)-1H-pyrazole

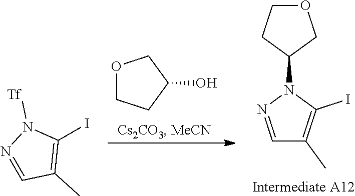

To a stirred solution of 5-Iodo-4-methyl-1-((trifluoromethyl) sulfonyl)-1H-pyrazole (1055 g, 3102 mmol) in MeCN (10.6 L) was added (R)-tetrahydrofuran-3-ol (301 g, 3413 mmol) followed by Cs₂CO₃ (2527 g. 7756 mmol) at 0° C. The resulting mixture was stirred for 16 h at rt, then diluted with water (5000 mL) and extracted with EtOAc (4000×2 mL). The organic extract was dried over Na₂SO₄. The solution was filtered and concentrated then purified by column chromatography, eluting with a gradient of 0% to 8% EtOAc in hexanes, to provide(S)-5-iodo-4-methyl-1-(tetrahydrofuran-3-yl)-1H-pyrazole, Intermediate A12, m/z (ESI): 279.1 (M+H)⁺. ¹H NMR (400 MHz, DMSO-d₆): δ 7.45 (s, 1H), 5.07-5.03 (m, 1H), 4.04-3.93 (m, 2H), 3.86-3.81 (m, 1H), 3.78-3.75 (m, 1H), 2.34-2.23 (m, 2H), 1.95 (s, 3H).

Intermediates in Table 1-4 were prepared following the procedure described for Intermediate A12, using appropriate starting materials. All starting materials are commercially available or are described above.

TABLE 1-4

| | Chemical Structure & Name | LCMS: (ESI + ve ion) m/z; NMR | Comments |
|---|---|---|---|
| A12-1 | 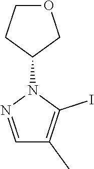 | m/z (ESI): 279.1 (M + H)⁺. | (R)-tetrahydrofuran- 3-ol was used |
| A13 | 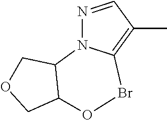 | m/z (ESI): 263.1 (M + H)⁺. | |
| | 5-bromo-1-(4- | | |
| | methoxytetrahydrofuuran-3- | | |
| | yl)-4-methyl-1H-pyrazole | | |

### Intermediate A14-5-Bromo-4-methyl-1-(3-methyloxetan-3-yl)-1H-pyrazole

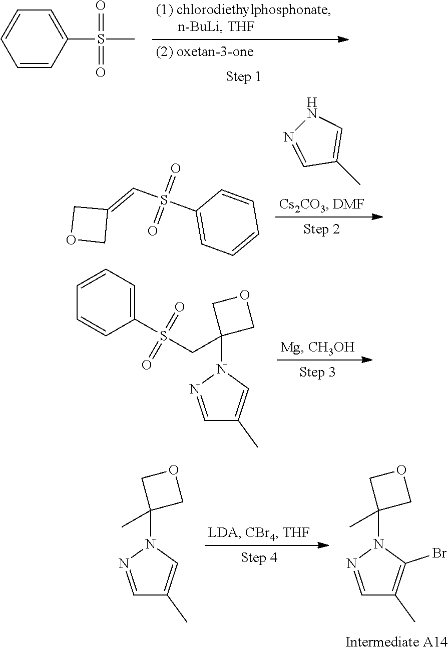

Step 1. 3-((Phenylsulfonyl)methylene)oxetane. To a stirred solution of (methylsulfonyl)benzene (100 g, 640 mmol) in THF (2000 mL) at 0° C. was added n-BuLi (2.5 M in hexanes, 563 mL, 1408 mmol) dropwise at 0° C., and stirred for 30 min. Chlorodiethylphosphonate (110 mL, 768 mmol) was added dropwise to the reaction mixture and stirred for 30 min at 0° C. The reaction mixture was cooled to −78° C., and a solution of oxetan-3-one (64.6 g, 896 mmol) in THF (500 mL) was added dropwise. The reaction mixture was stirred at −78° C. for 30 min. Then, the reaction mixture was quenched with satd. NH₄Cl solution (1000 mL) and extracted with EtOAc (3×3000 mL). The combined organic extracts were dried (Na₂SO₄), filtered, and concentrated. Hexanes (1000 mL) were added to the crude material, and the resulting precipitate was filtered, washed with hexanes (300 mL) and dried to give 3-((phenylsulfonyl)methylene)oxetane (134.0 g, 97% yield), m/z (ESI): 211.2 (M+H)⁺.

Step 2. 4-Methyl-1-(3-((phenylsulfonyl)methyl) oxetan-3-yl)-1H-pyrazole. To a stirred solution of 3-((phenylsulfonyl)methylene) oxetane (568.0 g, 2702 mmol) in DMF (5000 mL) was added 4-methyl-1H-pyrazole (266.0 g, 3242 mmol) at 0° C. under N₂ atmosphere. Cs₂CO₃ (1320 g, 4052 mmol) was added to the reaction mixture and stirred for 16 h at rt. The reaction mixture was quenched with ice cold water (1500 mL), and the precipitate was filtered, washed with water (3×3 L), and dried to give 4-methyl-1-(3-((phenylsulfonyl)methyl) oxetan-3-yl)-1H-pyrazole (550 g. 70% yield), m/z (ESI): 293.1 (M+H)⁺. ¹H NMR (400 MHz, DMSO-d₆): δ (ppm) 7.66-7.52 (m, 4H), 7.52-7.42 (m, 2H), 6.99 (s, 1H), 4.98 (d, J=7.2 Hz, 2H), 4.83 (d, J=7.3 Hz, 2H), 4.49 (s, 2H), 1.85 (s, 3H).

Step 3. 4-Methyl-1-(3-methyloxetan-3-yl)-1H-pyrazole. To a stirred solution of 4-methyl-1-(3-((phenylsulfonyl)methyl) oxetan-3-yl)-1H-pyrazole (75 g, 257 mmol) in MeOH (3000 mL) was added magnesium turnings (74.8 g, 3078 mmol) portion wise at rt. The reaction mixture was stirred at 50° C. for 30 min and at rt for 16 h. The reaction mixture was concentrated, quenched with 1.5 N HCl solution (6000 mL), and extracted with EtOAc (4×1500 mL). The combined organic extracts were washed with brine (3×1000 mL), dried (Na₂SO₄), filtered, and concentrated. The crude material was purified by flash chromatography, eluting with a gradient of 0 to 20% EtOAc in hexanes to give 4-methyl-1-(3-methyloxetan-3-yl)-1H-pyrazole (15.8 g, 41% yield), m/z (ESI): 153.1 (M+H)⁺. ¹H NMR (400 MHz, DMSO-d₆): 7.67 (t, J=0.9 Hz, 1H), 7.35 (s, 1H), 5.01-4.87 (m, 2H), 4.61-4.49 (m, 2H), 2.03 (t, J=0.7 Hz, 3H), 1.79 (d, J=0.6 Hz, 3H).

Step 4. 5-Bromo-4-methyl-1-(3-methyloxetan-3-yl)-1H-pyrazole, Intermediate A14. To a stirred solution of diisopropylamine (144 mL, 1030 mmol) in THF (840 mL) at −40° C., was added n-BuLi (2.5 M in hexanes, 368 mL, 920 mmol) dropwise and stirred for 10 min. The reaction mixture was warmed up to 0° C., and stirred for another 30 min. The reaction mixture was cooled down to −78° C., and a solution of 4-methyl-1-(3-methyloxetan-3-yl)-1H-pyrazole (56 g, 368 mmol) in THF (560 mL) was added dropwise and stirred for 60 min at −78° C. A solution of carbon tetrabromide (183 g, 552 mmol) in THF (560 mL) was added dropwise and stirred for 16 h at rt. The reaction mixture was quenched with satd. NH₄Cl solution (1000 mL) and extracted with EtOAc (3×1000 mL). The combined organic extracts were dried (Na₂SO₄), filtered, and concentrated under reduced pressure. The crude material was purified by chromatography, eluting with a gradient of 5-10% EtOAc in hexanes to give Intermediate A14 (34.0 g, 41% yield), m/2 (ESI): 231.0 and 233.1 (M+H)⁺. ¹H NMR (400 MHz, DMSO-d₆): δ (ppm) 7.47 (s, 1H), 5.10 (d, J=6.8 Hz, 2H), 4.58 (d, J=6.8 Hz, 2H), 1.95 (d, J=0.4 Hz, 3H), 1.72 (s, 3H).

### Intermediate A15-3-Chloro-2-(1-methoxycyclopropyl)-4-methylpyridine

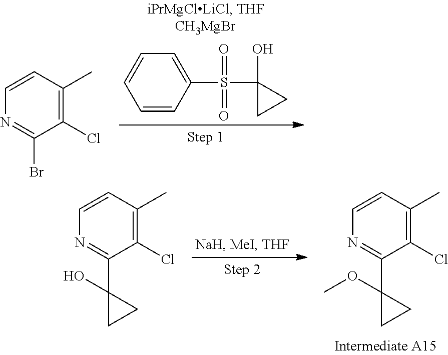

Step 1. 1-(3-Chloro-4-methylpyridin-2-yl)cyclopropan-1-ol. To a solution of 2-bromo-3-chloro-4-methylpyridine (5.76 g, 27.9 mmol, Combi-Blocks Inc.) in THF (25 mL) at 0° C. was added isopropylmagnesium chloride, lithium chloride complex (1.3 M solution in THE, 21.46 mL, 27.9 mmol, Sigma-Aldrich Corporation), and the reaction mixture was stirred at this temperature for 1 h. In a separate reaction vessel containing 1-(phenylsulfonyl)cyclopropan-1-ol (5.53 g, 27.9 mmol) and THF (30 mL) at −78° C. was added methylmagnesium bromide solution (3 M in ether, 9.30 mL, 27.9 mmol. Sigma-Aldrich Corporation) followed by the pyridyl Grignard solution prepared above. The reaction mixture was stirred at rt overnight. The reaction mixture was quenched by the addition of 1.0 M HCl and extracted with EtOAc. The aqueous phase was then washed with 1.0 M NaOH and extracted once more with EtOAc. The combined organic extracts were washed with brine, dried over Na₂SO₄, filtered, and concentrated. The crude material was purified by chromatography, eluting with a gradient of 0% to 60% EtOAc/EtOH (3:1) in heptane to provide 1-(3-chloro-4-methylpyridin-2-yl)cyclopropan-1-ol which was used in the next step without further purification, m/z (ESI): 184.2 (M+H)⁺.

Step 2. 3-Chloro-2-(1-methoxycyclopropyl)-4-methylpyridine, Intermediate A15. To a 0° C. suspension solution of NaH (0.650 g, 16.26 mmol, TCI America, 60% in mineral oil) in THF (10 mL) was added a solution of 1-(3-chloro-4-methylpyridin-2-yl)cyclopropan-1-ol (1.99 g. 10.84 mmol) in THF (10 mL) dropwise, and the reaction mixture was stirred at this temperature for 30 min. To the reaction mixture was added Mel (1.02 mL, 16.26 mmol, Sigma-Aldrich Corporation), and the reaction mixture was warmed to rt and stirred for 1 h. The reaction mixture was carefully quenched by the slow addition of water and was extracted with EtOAc. The organic extracts were washed with brine, dried over Na₂SO₄, filtered, and concentrated. The crude material was purified by chromatography, eluting with a gradient of 0% to 40% EtOAc in heptane, to provide Intermediate A15, m/z (ESI): 198.0 (M+H)⁺.

### Intermediate A16-1-(1-Methoxy-2-methylpropan-2-yl)-4-methyl-5-(4.4.5,5-tetramethyl-1,3,2-dioxaborolan-2-yl)-1H-pyrazole

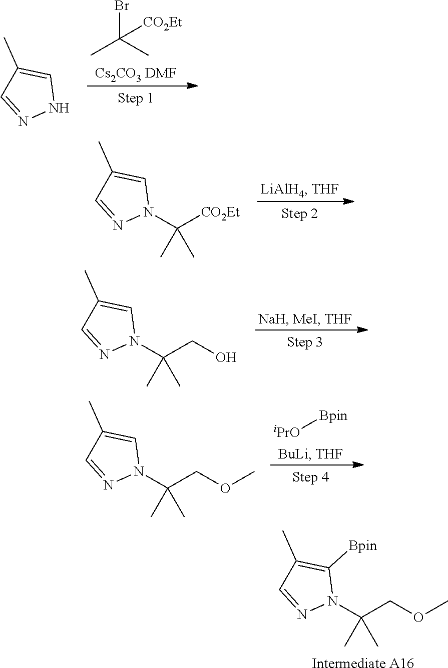

Step 1. Ethyl 2-methyl-2-(4-methyl-1H-pyrazol-1-yl)propanoate. To a solution of 4-methyl-1H-pyrazole (4 g, 48.7 mmol. Ambeed, Inc.) in DMF (100 mL) was added Cs₂CO₃ (18.53 g, 56.9 mmol, Sigma-Aldrich Corporation), followed by ethyl 2-bromo-2-methylpropanoate (7.87 mL, 53.6 mmol. Sigma-Aldrich Corporation). The mixture was stirred at 60° C. for 2 h. The solid was filtered off and rinsed with EtOAc. The filtrate was concentrated to remove the majority of DMF and partitioned between EtOAc and water. The aqueous layer was extracted with EtOAc (3×20 mL). The combined organic extracts were washed with brine and concentrated in vacuo to provide crude ethyl 2-methyl-2-(4-methyl-1H-pyrazol-1-yl)propanoate, which was taken to next step, m/z (ESI): 197.1 (M+H)+

Step 2. 2-Methyl-2-(4-methyl-1H-pyrazol-1-yl)propan-1-ol. To a 0° C. solution of ethyl 2-methyl-2-(4-methyl-1H-pyrazol-1-yl)propanoate (4 g, 12.23 mmol) in THF (60 mL) was added dropwise LAH (2 M solution in THF, 6.73 mL, 13.45 mmol, Sigma-Aldrich Corporation), and stirred for 30 min. The reaction was quenched with water, then diluted with 1 N NaOH (10 mL). After stirring for 15 min, the reaction was filtered, and the filtrate was diluted with brine, separated, and concentrated. The crude product was eluted through a short plug of silica gel using EtOAc, and the filtrate was concentrated to give 2-methyl-2-(4-methyl-1H-pyrazol-1-yl)propan-1-ol (1.88 g, 12.19 mmol, yield: 99%), m/z (ESI): 155.1 (M+H)⁺.

Step 3. 1-(1-Methoxy-2-methylpropan-2-yl)-4-methyl-1H-pyrazole. To a 0° C. solution of 2-methyl-2-(4-methyl-1H-pyrazol-1-yl)propan-1-ol (1.886 g. 12.23 mmol) in THE (60 mL) was added NaH, 60 wt % in mineral oil (0.734 g, 18.34 mmol, TCI America) in two portions. After stirring for 30 min. Mel (2.40 g, 16.87 mmol, Sigma-Aldrich) was added at a fast drip, and the reaction mixture was stirred for 45 min. The reaction mixture was removed from the cooling bath and allowed to stir for an additional 30 min, before quenched with aqueous satd. NH₄Cl. The reaction mixture was partitioned between EtOAc and water. The aqueous layer was extracted with EtOAc (3×15 mL). The combined organic extracts were concentrated and purified via chromatography, eluting with 0-50% EtOAc: EtOH (3:1) in heptane, to provide 1-(1-methoxy-2-methylpropan-2-yl)-4-methyl-1H-pyrazole, m/z (ESI): 169.2 (M+H)⁺.

Step 4. 1-(1-Methoxy-2-methylpropan-2-yl)-4-methyl-5-(4,4,5,5-tetramethyl-1,3,2-dioxaborolan-2-yl)-1H-pyrazole, Intermediate A16. To a solution of 1-(1-methoxy-2-methylpropan-2-yl)-4-methyl-1H-pyrazole (0.214 g, 1.272 mmol) in THF (4 mL) at 0° C. was added n-BuLi (2.5 M solution in hexanes, 1.53 mL. 3.82 mmol, Sigma-Aldrich Corporation) dropwise over 1 min. After stirring for 30 min at 0° C., the solution was cooled to −78° C., and treated with 2-isopropoxy-4,4,5,5-tetramethyl-1,3,2-dioxaborolane (0.78 mL, 3.82 mmol, Sigma-Aldrich Corporation) dropwise. The reaction mixture was allowed to warm to rt, quenched with water, and partitioned between EtOAc and water. The aqueous layer was extracted with EtOAc (3×15 mL). The combined organic extracts were washed with brine and concentrated in vacuo. The crude Intermediate A16 was carried forward as is, m/z (ESI): 295.2 (M+H)⁺.

### Intermediate A17-6-(3-Bromo-5-fluoro-4-methylpyridin-2-yl)-2-oxa-6-azaspiro[3.3]heptane

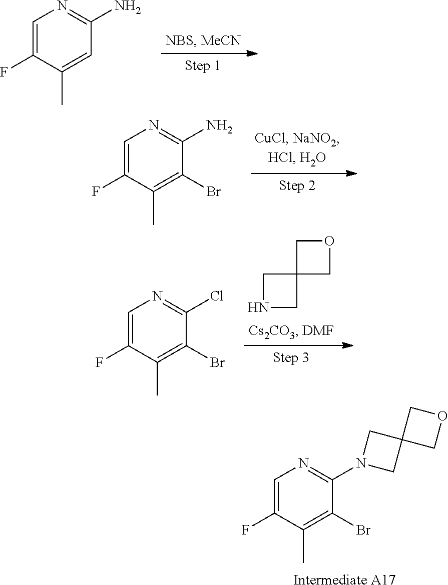

Step 1. 3-Bromo-5-fluoro-4-methylpyridin-2-amine. To a suspension of 2-amino-5-fluoro-4-picoline (1.02 g, 8.09 mmol, AK Scientific, Inc.) in MeCN (10 mL) was added NBS (1.44 g, 8.09 mmol, Sigma-Aldrich Corporation), and the resulting mixture was stirred at rt for 2 h. The reaction was concentrated and purified via chromatography, eluting with a gradient of 0-20% (3:1 EtOAc/EtOH) in heptane to afford 3-bromo-5-fluoro-4-methylpyridin-2-amine, m/z (ESI): 205.0 (M+H)⁺. ¹H NMR (400 MHz, DMSO-d₆) δ (ppm) 7.90 (s, 1H), 6.04 (br s, 2H), 2.25 (d, J=2.1 Hz, 3H).

Step 2. 3-Bromo-2-chloro-5-fluoro-4-methylpyridine. To a mixture of conc. HCl (6 mL) and water (6 mL) was added 3-bromo-5-fluoro-4-methylpyridin-2-amine (0.801 g, 3.91 mmol), followed by NaNO₂ (0.809 g, 11.72 mmol, Sigma-Aldrich Corporation) and CuCl (1.547 g, 15.63 mmol, Strem Chemicals, Inc.), and the resulting mixture was stirred at rt for 18 h. The reaction was brought to 0° C., diluted with EtOAc and basified with 1 N NaOH. The mixture was filtered to remove the solids, and the filtrate was extracted with EtOAc. The combined organics were dried over Na₂SO₄, filtered, and concentrated to afford 3-bromo-2-chloro-5-fluoro-4-methylpyridine that was taken directly to next step, m/z (ESI): 223.9 (M+H)⁺. ¹H NMR (500 MHz, DMSO-d₆) δ (ppm) 8.45 (s, 1H), 2.40 (d, J=2.2 Hz, 3H).

Step 3. 6-(3-Bromo-5-fluoro-4-methylpyridin-2-yl)-2-oxa-6-azaspiro[3.3]heptane, Intermediate A17. A mixture of 3-bromo-2-chloro-5-fluoro-4-methylpyridine (0.313 g, 1.394 mmol), 2-oxa-6-azaspiro[3.3]heptane (0.166 g, 1.67 mmol, Advanced ChemBlocks Inc.), and Cs₂CO₃ (1.36 g, 4.18 mmol, Combi-Blocks Inc.) in DMF (10 mL) was heated at 98° C. for 5 h. The reaction was diluted with water and extracted with EtOAc. The combined organics extracts were dried over Na₂SO₄, filtered, concentrated, and chromatographed using 0-30% EtOAc in heptane to afford 6-(3-bromo-5-fluoro-4-methylpyridin-2-yl)-2-oxa-6-azaspiro[3.3]heptane (56 mg, 0.20 mmol, yield: 14%), Intermediate A17, m/z (ESI): 287.0 (M+H)⁺.

### Intermediate A18-3-Chloro-4-methyl-2-(oxetan-3-yl)pyridine

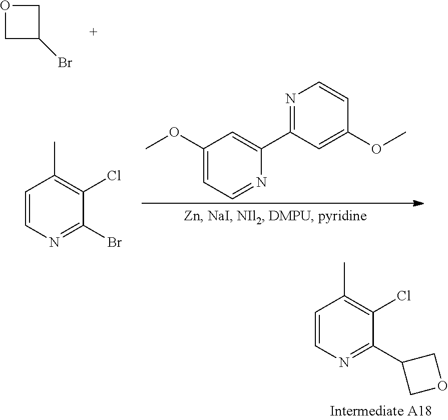

Step 1. 3-Chloro-4-methyl-2-(oxetan-3-yl)pyridine, Intermediate A18. To a mixture of nickel (II) iodide hydrate (0.469 g, 1.114 mmol, Combi-Blocks Inc.), 4,4-dimethoxy-2,2-bipyridine (0.241 g, 1.114 mmol, Sigma-Aldrich Corporation), NaI (0.417 g, 2.78 mmol, Sigma-Aldrich Corporation), 2-bromo-3-chloro-4-methylpyridine (2.30 g, 11.14 mmol, Combi-Blocks Inc.), and zinc dust (1.457 g, 22.28 mmol, Sigma-Aldrich Corporation) was added pyridine (0.090 mL, 1.114 mmol, Sigma-Aldrich Corporation) and DMPU (20 mL). After stirring at rt for 5 min, the mixture was heated at 60° C. overnight. The reaction mixture was diluted with EtOAc and filtered. The filtrate was washed with water, and the organic phase was dried over Na₂SO₄, filtered, concentrated, and purified via chromatography, eluting using a gradient of 0-30% EtOAc in heptane to afford Intermediate A18, m/z (ESI): 184.2 (M+H)⁺.

### Intermediate A19-(28,3S)-2-Allylazetidin-3-ol

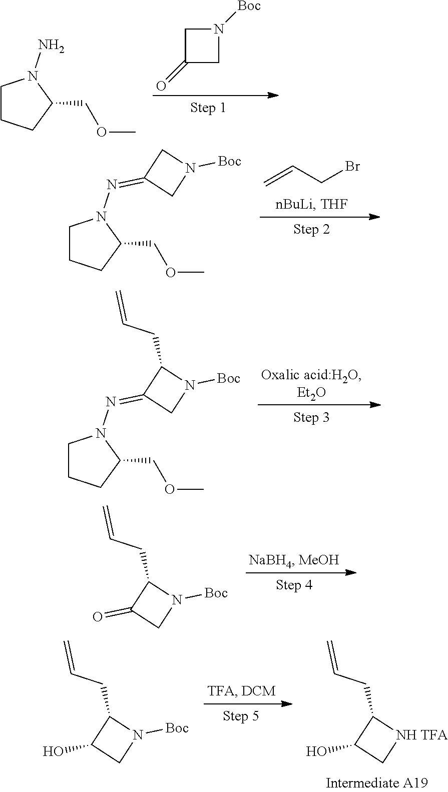

Step 1. tert-Butyl(S)-3-((2-(methoxymethyl) pyrrolidin-1-yl)imino)azetidine-1-carboxylate. (S)-2-(methoxymethyl) pyrrolidin-1-amine (7.99 g, 61.3 mmol) was added dropwise to tert-butyl 3-oxoazetidine-1-carboxylate (10 g, 58.4 mmol). The reaction mixture was warmed to 70° C. for 16 h and concentrated under vacuum to give tert-butyl(S)-3-((2-(methoxymethyl) pyrrolidin-1-ylimino)azetidine-1-carboxylate (16 g, 56.5 mmol, 97% yield), m/z (ESI): 284.3 (M+H)⁺.

Step 2. tert-Butyl(S,E)-2-allyl-3-(((S)-2-(methoxymethyl) pyrrolidin-1-yl)imino)azetidine-1-carboxylate. To a solution of tert-butyl(S)-3-((2-(methoxymethyl) pyrrolidin-1-yl)imino)azetidine-1-carboxylate (16.0 g, 56.5 mmol) in THF (240 mL) at −78° C. was added n-BuLi (24.84 mL, 62.1 mmol, 2.5 M in hexanes) via dropwise addition. The reaction mixture was stirred for 2 h at −78° C. Then, allyl bromide (5.86 mL, 67.8 mmol) was added at −78° C., and the reaction mixture was gradually warmed to rt and stirred for 6 h. The reaction mixture was diluted with diethyl ether, and water was added at 0° C., and the product was extracted with diethyl ether (2×100 mL). The combined organic extracts were concentrated to give tert-butyl(S,E)-2-allyl-3-(((S)-2-(methoxymethyl) pyrrolidin-1-yl)imino)azetidine-1-carboxylate (17 g), which was taken to next step without purification, m/z (ESI): 324.3 (M+H)⁺.

Step 3. tert-Butyl(S)-2-allyl-3-oxoazetidine-1-carboxylate. To a solution of tert-butyl(S,E)-2-allyl-3-(((S)-2-(methoxymethyl) pyrrolidin-1-yl)imino)azetidine-1-carboxylate (17 g, 21.02 mmol) in diethyl ether (170 mL) was added oxalic acid (satd. solution in water, 85 mL, 25.2 mmol). The reaction mixture was stirred at 25° C. for 16 h, then diluted with water, extracted with diethyl ether (2×100 mL), dried, and concentrated. The crude material was purified by chromatography, eluting with a gradient of 5% to 10% EtOAc in hexanes, to provide tert-butyl(S)-2-allyl-3-oxoazetidine-1-carboxylate (2.5 g, 11.83 mmol, 56% yield), m/z (EST): 112.1 (M-Boc+H)⁺.

Step 4. tert-Butyl(2S,3S)-2-allyl-3-hydroxyazetidine-1-carboxylate. To a stirred solution of tert-butyl(S)-2-allyl-3-oxoazetidine-1-carboxylate (2.5 g, 11.83 mmol) in MeOH (25.00 ml) at 0° C. was slowly added NaBH₄ (0.895 g. 23.67 mmol), and the resulted reaction mixture was stirred for 16 h at 25° C. The reaction mixture was quenched with satd. NH₄Cl and extracted with EtOAc (3×20 mL). The organic extracts were combined, washed with brine, dried, filtered, and concentrated. The crude residue was purified by chromatography, eluting with a gradient of 10-50% EtOAc in pet. ether to give ter-butyl(25,3S)-2-allyl-3-hydroxyazetidine-1-carboxylate (2.5 g, 11.72 mmol, 99% yield), m/z (ESI): 114.2 (M-Boc+H)⁺. 3H NMR (400 MHz, CDCl₃ δ 5.95 (ddt, J=17.1, 10.3, 6.7 Hz, 1H), 5.28-5.03 (m, 2H), 4.64 (s, 1H), 4.43-4.28 (m, 1H), 4.22-3.93 (m, 1H), 3.71 (ddd, J=9.8, 4.3, 1.1 Hz, 1H), 2.82-2.51 (m, 2H), 2.21 (s, 1H), 1.67 (s, 2H), 1.46 (d, J=2.6 Hz, 9H).

Step 5. (25,3S)-2-Allylazetidin-3-ol trifluoroacetate, Intermediate A19. To a stirred solution of tort-butyl(2S,3S)-2-allyl-3-hydroxyazetidine-1-carboxylate (2.5 g, 11.72 mmol) in DCM (5 mL) at 0° C. was added TFA (9.03 mL, 117 mmol) dropwise, and the resulted solution stirred at 25° C. for 2 h. Volatiles were evaporated under reduced pressure to give the crude product, which was used for the next step without purification, m/z (ESI): 114.2 (M+H)⁺.

### Intermediate A20-3-Bromo-4,6-dichloro-2-(trifluoromethyl)pyridine

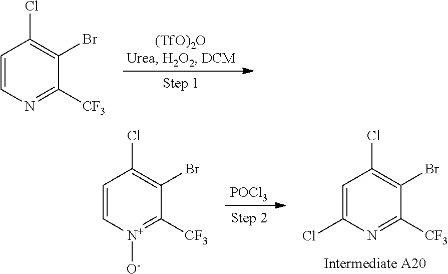

Step 1. 3-Bromo-4-chloro-2-(trifluoromethyl)pyridine 1-oxide. To the solution of 3-bromo-4-chloro-2-(trifluoromethyl)pyridine (25 g, 96 mmol) and urea hydrogen peroxide (90 g, 960 mmol) in DCM (500 mL) was added trifluoroacetic anhydride (133 mL, 960 mmol) dropwise at 0° C. The reaction mixture was stirred at rt for 20 h. Then, the reaction mixture was quenched with 10% sodium metabisulphite solution (1000 mL) and extracted with DCM (3×500 mL). The combined organic extracts were washed with water (1000 mL) followed by brine (1000 mL), dried, and concentrated. The crude residue was purified by chromatography, eluting with a gradient of 18-26% EtOAc in hexanes to give 3-bromo-4-chloro-2-(trifluoromethyl)pyridine 1-oxide.

Step 2. 3-Bromo-4,6-dichloro-2-(trifluoromethyl)pyridine, Intermediate A20. To 3-bromo-4-chloro-2-(trifluoromethyl)pyridine 1-oxide (39 g, 141 mmol) was added POCl₃ (276 mL, 2963 mmol) at 0° C. The reaction mixture was heated at 100° C. for 10 h, and then quenched with a slurry of ice and NaHCO₃. The aqueous layer was extracted with DCM (3×750 mL), dried over anhydrous Na₂SO₄, and concentrated to give Intermediate A20. ¹H NMR (400 MHz, DMSO-d₆) δ 8.38 (s, 1H).

### Intermediate: tert-Butyl(R)-6-methyl-4-(4,4,5,5-tetramethyl-1,3,2-dioxaborolan-2-yl)-3,6-dihydropyridine-1 (2H)-carboxylate

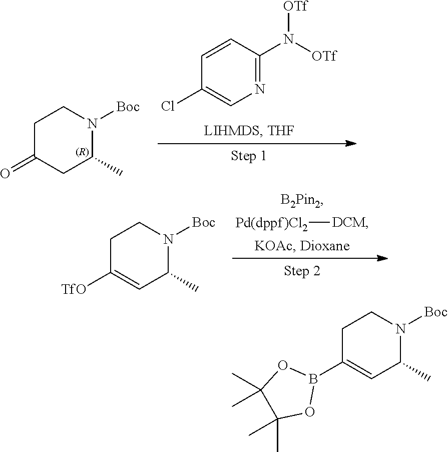

Step 1. tert-Butyl(R)-6-methyl-4-(((trifluoromethyl)sulfonyl)oxy)-3,6-dihydropyridine-1 (2H)-carboxylate. To a solution of tert-butyl(R)-2-methyl-4-oxopiperidine-1-carboxylate (25 g, 117 mmol) in THF (250 mL) at −78° C. under N₂ atmosphere was added LiHMDS (1 M solution in THE, 176 mL, 176 mmol). The temperature of the reaction mixture was raised to 0° C., and a solution of N-(5-chloropyridin-2-yl)-1,1,1-trifluoro-N-((trifluoromethyl) sulfonyl) methanesulfonamide (59.8 g, 152 mmol) in THF (125 mL) was added dropwise and stirred at rt for 30 min. The reaction mixture was quenched with satd. aq. NH₄Cl solution (300 mL) and extracted with EtOAc (300 mL). The organic extract was washed with brine (200 mL), dried over anhydrous Na₂SO₄, and concentrated under vacuum. The crude residue was purified by chromatography, eluting with a gradient of 5-7% EtOAc in pet. ether to afford tert-butyl(R)-6-methyl-4-(((trifluoromethyl)sulfonyl)oxy)-3,6-dihydropyridine-1 (2H)-carboxylate (30 g, 87 mmol, 74% yield), m/z (ESI): 246.0 (M-Boc+H)⁺.

Step 2. tert-Butyl(R)-6-methyl-4-(4,4,5,5-tetramethyl-1,3,2-dioxaborolan-2-yl)-3,6-dihydropyridine-1 (2H)-carboxylate. To a solution of tert-butyl(R)-6-methyl-4-(((trifluoromethyl) sulfonyl)oxy)-3,6-dihydropyridine-1 (2H)-carboxylate (30 g, 87 mmol) in 1,4-dioxane (600 mL) were added B₂Pin₂ (24.27 g, 96 mmol) and KOAc (25.6 g, 261 mmol) at rt. The reaction mixture was degassed for 5 min, and then, Pd(dppf)Cl₂-DCM adduct (3.55 g. 4.34 mmol) was added. The solution was heated to 90° C. overnight. The reaction mixture was concentrated and purified by chromatography, eluting with a gradient of 5-10% EtOAc in pet. ether to give the desired product (15 g, 46.4 mmol, 53% yield), m/z (ESI): 224.2 (M-Boc+H)⁺.

### Intermediate B1-2-(3-Methoxyoxetan-3-yl)-4-methyl-3-(piperidin-4-yl)pyridine

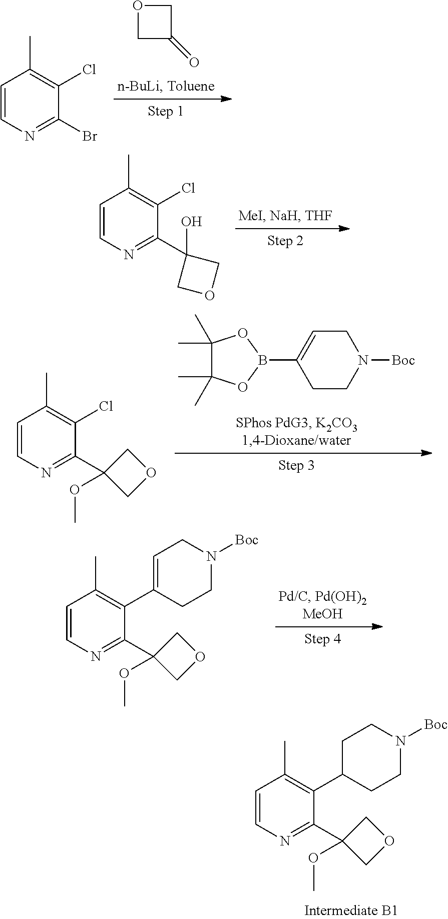

Step 1. 3-(3-Chloro-4-methylpyridin-2-yl) oxetan-3-ol. To a stirred solution of 2-bromo-3-chloro-4-methylpyridine (600 g, 2906 mmol) in toluene (9 L) at −78° C. was added n-butyllithium (2.5 M solution in hexanes, 1162 mL, 2906 mmol) dropwise for 60 min, and reaction mixture was stirred for 1 h at −78° C. A solution of oxetan-3-one (188 g, 2615 mmol) in toluene (300 mL) was added over 30 min and stirred for 1 h at −78° C. The reaction mixture was quenched with satd. aq. NH₂Cl (10 L). The aqueous layer was extracted with EtOAc (3×4 L). The combined organic extracts were washed with brine (5 L), dried over Na₂SO₄, filtered, and concentrated. Then, hexanes (3 L) was added, the mixture was concentrated, filtered and dried to give 3-(3-chloro-4-methylpyridin-2-yl) oxetan-3-ol, m/z (ESI): 200.1 (M+H)⁺. 3H NMR (400 MHz, DMSO-d₆): δ (ppm) 8.36 (d, J=4.8 Hz, 1H), 7.40 (dd, J=4.9, 0.9 Hz, 1H), 6.46 (s, 1H), 5.15 (dd, J=6.9, 1.0 Hz, 2H), 4.69 (dd, J=6.9, 1.0 Hz, 2H), 2.38 (d, J=0.7 Hz, 3H).

Step 2. 3-Chloro-2-(3-methoxyoxetan-3-yl)-4-methylpyridine. To a stirred solution of 3-(3-chloro-4-methylpyridin-2-yl) oxetan-3-ol (260 g, 1302 mmol) in THF (2.5 L) at 0° C. was added NaH (104 g, 2605 mmol, 60% in mineral oil) portion wise for 15 min, and the reaction mixture was stirred at 0° C. for 30 min. Methyl iodide (163 mL, 2605 mmol) was added over 20 min, and the reaction mixture was slowly warmed to rt and stirred for 16 h. The reaction was quenched with ice water (10 L) and extracted with EtOAc (2×5 L). The combined organic extracts were dried over Na₂SO₄ and concentrated to give 3-chloro-2-(3-methoxyoxetan-3-yl)-4-methylpyridine, m/z (ESI): 214.3 (M+H)⁺. 1H NMR (400 MHz, DMSO-d₆): δ (ppm) 8.42 (d, J=4.9 Hz, 1H), 7.46 (dd, J=4.9, 0.8 Hz, 1H), 5.08 (dd. J=7.3, 1.1 Hz, 2H), 4.76 (dd, J=7.4, 1.1 Hz, 2H), 2.97 (s, 3H), 2.40 (d, J=0.7 Hz, 3H).

Step 3. tert-Butyl 2-(3-methoxyoxetan-3-yl)-4-methyl-3′,6′-dihydro-[3,4′-bipyridine]-1′(2′H)-carboxylate. A solution of 3-chloro-2-(3-methoxyoxetan-3-yl)-4-methylpyridine (344 g, 1610 mmol), tert-butyl 4-(4,4,5,5-tetramethyl-1,3,2-dioxaborolan-2-yl)-3,6-dihydropyridine-1 (2H)-carboxylate (597 g, 1932 mmol), K₂CO₃ (668 g, 4830 mmol) in 1,4-dioxane (3440 mL), water (1032 mL) was stirred at rt. The reaction mixture was degassed for 5 min, and SPhos Pd G3 (62.8 g, 81 mmol) was added. The reaction mixture was heated to 100° C. for 16 h. The reaction mixture was concentrated, diluted with water (5 L), and extracted with EtOAc (3×5 L). The combined organic extracts were dried over Na₂SO₄ and filtered, and concentrated. The crude material was purified by chromatography, eluting with a gradient of 25% to 50% EtOAc in hexanes, to give tert-butyl 2-(3-methoxyoxetan-3-yl)-4-methyl-3′,6′-dihydro-[3,4′-bipyridine]-1′(2′H)-carboxylate (500 g, 1387 mmol, 86% yield), m/z (ESI): 361.3 (M+H)⁺. ¹H NMR (400 MHz, DMSO-d₆): δ (ppm) 8.35 (d, J=4.9 Hz, 1H), 7.28 (dd, J=4.9, 0.8 Hz, 1H), 5.49 (s, 1H), 5.25 (d, J=7.2 Hz, 1H), 4.86 (d,)=7.3 Hz, 1H), 4.39-4.63 (m, 2H), 3.92 (d, J=5.2 Hz, 2H), 3.51 (t, J=5.3 Hz, 2H), 2.94 (s, 3H), 2.43-2.01 (m, 5H), 1.44 (s, 9H).

Step 4. tert-Butyl 4-(2-(3-methoxyoxetan-3-yl)-4-methylpyridin-3-yl)piperidine-1-carboxylate, Intermediate BI. To a stirred solution of tert-butyl 2-(3-methoxyoxetan-3-yl)-4-methyl-3′,6′-dihydro-[3,4′-bipyridine]-1′(2′H)-carboxylate (60 g, 166 mmol) in MeOH (1000 mL) at rt was added 10% palladium on activated carbon (50% wet) (30 g, 28.2 mmol) and 20% palladium (II) hydroxide on carbon (30 g, 214 mmol), and the reaction mixture was degassed thoroughly and stirred under H₂ balloon pressure for 5 d at rt. The reaction mixture was filtered, washed with 50% DCM in MeOH (2000 mL), and concentrated. The crude was purified via SFC using a ChiralPak IG (250×50) mm, 5 μm, column with a mobile phase of liquid CO₂: [0.2% NH₃ in ACN: EtOH (2:8)] using a flowrate of 150 mL/min to give Intermediate B1, m/z (ESI): 363.1 (M+H)⁺. ¹H NMR (400 MHz, DMSO-d₆): δ (ppm) 8.28 (d, J=4.9 Hz, 1H), 7.18 (d, J=5.0 Hz, 1H), 5.11 (d, J=7.1 H), 4.79 (dd, J=7.0, 1.0 Hz, 2H), 4.05 (d, J=12.9 Hz, 2H), 2.93 (s, 3H), 2.55-2.80 (m, 3H), 2.42 (s, 3H), 1.93 (qd, J=12.6, 4.3 Hz, 2H), 1.50 (d, J=12.8 Hz, 2H), 1.42 (s, 9H).

Intermediates in Table 1-5 were prepared following the procedure described for Intermediate B1, using appropriate starting materials. All starting materials are commercially available or are described above.

TABLE 1-5

| | Chemical Structure & Name | LCMS: (ESI + ve ion) m/z; NMR | Comments |
|---|---|---|---|
| B2 | 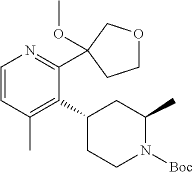 [SMILES: COC1(c2nccc(C)c2[C@H]2CCN(C(=O)OC(C)(C)C)[C@H](C)C2)CCOC1] | m/z (ESI): 391.2 (M + H)⁺. | Purification by prep HPLC: Discovery C- 18 column (250*21.2) nm 5.0 μm using a mobile phase A: 0.1% formic acid in water, B: ACN, flow rate: 15 mL/min. |
| | tert-butyl (2R,4S)-4-(2-(3- | | |
| | methoxytetrahydrofuran-3- | | |
| | yl)-4-methylpyridin-3-yl)-2- | | |
| | methylpiperidine-1- | | |
| | carboxylate | | |
| B3 | 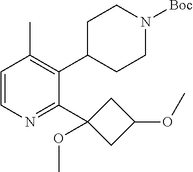 [SMILES: COC1CC(OC)(c2nccc(C)c2C2CCN(C(=O)OC(C)(C)C)CC2)C1] | m/z (ESI): 391.3 (M + H)⁺. | |
| | tert-butyl 4-(2-(1,3- | | |
| | dimethoxycyclobutyl)-4- | | |
| | methylpyridin-3- | | |
| | yl)piperidine-1-carboxylate | | |

### Intermediate B4-tert-Butyl 4-(1-(1-methoxycyclopropyl)-4-methyl-1H-pyrazol-5-yl)piperidine-1-carboxylate

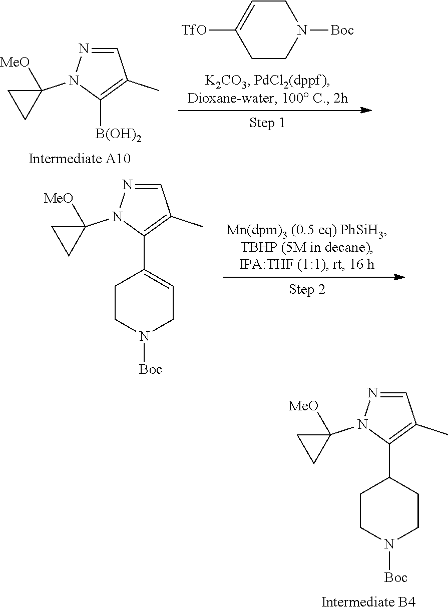 [SMILES: COC1(n2ncc(C)c2B(O)O)CC1 ; CC(C)(C)OC(=O)N1CC=C(OS(=O)(=O)C(F)(F)F)CC1 ; COC1(n2ncc(C)c2C2=CCN(C(=O)OC(C)(C)C)CC2)CC1 ; COC1(n2ncc(C)c2C2CCN(C(=O)OC(C)(C)C)CC2)CC1]

Step 1. tert-Butyl 4-(1-(1-methoxycyclopropyl)-4-methyl-1H-pyrazol-5-yl)-3,6-dihydropyridine-1 (2H)-carboxylate. A mixture of (1-(1-methoxycyclopropyl)-4-methyl-1H-pyrazol-5-yl) boronic acid (50 g, 255 mmol), tert-butyl 4-(((trifluoromethyl)sulfonyl)oxy)-3,6-dihydropyridine-1 (2H)-carboxylate (127 g, 383 mmol), and potassium carbonate (70.5 g, 510 mmol) in 1,4-dioxane (750 mL) and water (75 mL) was degassed and purged with N₂ for 15 min. To this solution was added PdCl₂ (dppf) (9.33 g, 12.75 mmol) and stirred at 110° C. for 16 h. The reaction mixture was quenched with water (500 mL) and extracted with EtOAc (2×600 mL). The combined organic extracts were washed with brine (600 mL), dried (Na₂SO₄), filtered, and concentrated under reduced pressure. The crude material was purified by chromatography, eluting with a gradient of 15% to 20% EtOAc in hexanes, to give tert-butyl 4-(1-(1-methoxycyclopropyl)-4-methyl-1H-pyrazol-5-yl)-3,6-dihydropyridine-1 (2H)-carboxylate (45 g, 135 mmol, 53% yield), m/z (ESI): 334.1 (M+H)⁺. ¹H NMR (400 MHz, DMSO-d₆): δ 7.27 (s, 1H), 5.86 (s, 1H), 3.99 (d, J=3.6 Hz, 2H), 3.52 (t, J=5.6 Hz, 2H), 3.21 (s, 3H), 2.39 (dt, J=7.7, 4.0 Hz, 2H), 1.94 (s, 3H), 1.44 (s, 9H), 1.22-1.28 (m, 2H), 1.12-1.17 (m, 2H).

Step 2. tert-Butyl 4-(1-(1-methoxycyclopropyl)-4-methyl-1H-pyrazol-5-yl)piporidine-1-carboxy late. To a stirred solution of tert-butyl 4-(1-(1-methoxycyclopropyl)-4-methyl-1H-pyrazol-5-yl)-3,6-dihydropyridine-1 (2H)-carboxylate (40 g, 120 mmol) in THF (400 mL) and IPA (400 mL) were added tris(2,2,6,6-tetramethyl-3,5-heptanedionato) manganese (III) (36.3 g, 60.0 mmol) and phenyl silane (64.9 g, 600 mmol) in a stepwise manner at 0° C. under N₂ atmosphere. The reaction mixture was stirred at 0° C. for 15 min. To the reaction mixture was added tert-butyl hydroperoxide (SM solution in decane, 120 mL, 600 mmol) dropwise by maintaining the temperature at 0° C., and stirred at rt for 16 h. The reaction mixture was quenched with ice cold water (500 mL) and satd. aq. sodium metabisulfite solution (500 mL) and extracted with EtOAc (2×1000 mL). The combined organic extracts were washed with brine (3×1000 mL), dried (Na₂SO₄), filtered and concentrated under reduced pressure. The crude material was purified by chromatography (elution: 15% to 30% EtOAc in hexanes) to give tert-butyl 4-(1-(1-methoxycyclopropyl)-4-methyl-1H-pyrazol-5-yl)piperidine-1-carboxylate (Intermediate B4) (5.7 g, 16.99 mmol, 14% yield), m/z (ESI): 336.3 (M+H)⁺. ¹H NMR (400 MHz, DMSO-d₆): δ 7.17 (s, 1H), 4.07 (d, J=13.2 Hz, 2H), 3.28-3.39 (m, 1H), 3.05 (s, 3H), 2.77 (br s, 2H), 2.02 (s, 3H), 1.64-1.73 (m, 4H), 1.42 (s, 9H), 1.33 (dt, J=6.3, 3.5 Hz, 2H), 1.25-1.27 (m, 2H).

### Intermediate B5-tert-Butyl 4-(2-(1-methoxycyclopropyl)-4-methylpyridin-3-yl)piperidine-1-carboxylate

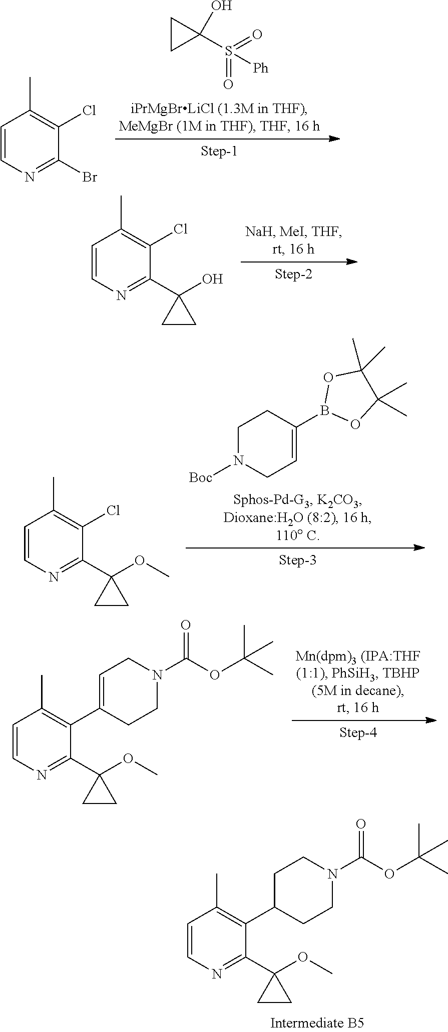

Step 1. tert-Butyl 2-(1-methoxycyclopropyl)-4-methyl-3′,6′-dihydro-[3,4′-bipyridine]-1′(2′H)-carboxylate. To a stirred solution of 3-chloro-2-(1-methoxycyclopropyl)-4-methylpyridine (26 g. 132 mmol) in 1,4-dioxane (3440 mL) and water (1032 mL), were added tert-butyl 4-(4,4,5,5-tetramethyl-1,3,2-dioxaborolan-2-yl)-3,6-dihydropyridine-1 (2H)-carboxylate (48.8 g, 158 mmol) and K₂CO₃ (54.5 g, 395 mmol) at rt. The reaction mixture was degassed and purged with N₂ for 5 min. SPhos Pd G; (5.13 g, 6.58 mmol) was added and the reaction mixture was stirred at 100° C. for 16 h. The reaction mixture was concentrated under reduced pressure and the crude was quenched with water (500 mL) and extracted with EtOAc (3×500 mL). The combined organic extracts were dried (Na₂SO₄), filtered, and concentrated under reduced pressure. The crude material was purified by flash chromatography (Redi-Sep pre-packed silica gel column, elution: 15 to 20% EtOAc in hexanes) to give tert-butyl 2-(1-methoxycyclopropyl)-4-methyl-3′,6′-dihydro-[3,4′-bipyridine]-1′(2′H)-carboxylate (18 g, 52.3 mmol, 40% yield), m/z (ESI): 345.3 (M+H)⁺. 3H NMR (400 MHz, DMSO-d₆): δ 8.24 (d, J=4.9 Hz, 1H), 7.18 (dd, J=4.9, 0.8 Hz, 1H), 5.50 (s, 1H), 4.01 (d, J=14.6 Hz, 1H), 3.88 (d, J=14.6 Hz, 1H), 3.67-3.44 (m, 2H), 2.99 (s, 3H), 2.37 (d, J=16.1 Hz, 1H), 2.22 (s, 3H), 2.19 (d, J=16.1 Hz, 1H), 1.19 (d, J=16.8 Hz, 1H), 1.08 (dd, J=4.4, 2.9 Hz, 2H).

Step 2. tert-Butyl 4-(2-(1-methoxycyclopropyl)-4-methylpyridin-3-yl)piperidine-1-carboxylate (Intermediate B5). To a stirred solution of tert-butyl 2-(1-methoxycyclopropyl)-4-methyl-3′,6′-dihydro-[3,4′-bipyridine]-1′(2′H)-carboxylate (25 g, 72.6 mmol) in THF (250 mL) and IPA (250 mL) were added tris(2.2.6,6-tetramethyl-3,5-heptanedionato) manganese (III) (21.95 g, 36.3 mmol) and phenyl silane (44.7 mL, 363 mmol) in a stepwise manner at 0° C. under N₂ atmosphere. The reaction mixture was stirred for 15 min at 0° C., and tert-butyl hydroperoxide (5 M solution in decane, 72.6 mL, 363 mmol) was added dropwise by maintaining the temperature at 0° C. The reaction mixture was stirred at rt for 16 h. The reaction mixture was quenched with ice cold water (500 mL) and satd. aq. sodium metabisulfite solution (500 mL) and extracted with EtOAc (2×500 mL). The combined organic extracts were washed with brine (2×100 mL), dried (Na₂SO₄), filtered, and concentrated under reduced pressure. The crude material was purified by reverse phase MPLC (elution: 30% to 50% ACN in water) to give tert-butyl 4-(2-(1-methoxycyclopropyl)-4-methylpyridin-3-yl)piperidine-1-carboxylate (Intermediate B5) (12.5 g, 36.1 mmol, 50% yield), m/z (ESI): 347.4 (M+H)⁺. 3H NMR (400 MHz, DMSO-d₆): δ 8.19 (d, J=4.9 Hz, 1H), 7.10 (d, J=5.0 Hz, 1H), 4.10 (d, J=13.1 Hz, 2H), 3.83 (d, J=13.1 Hz, 1H), 3.00 (s, 3H), 2.79 (br s, 2H), 2.42 (s, 3H), 1.98 (qd, J=12.6, 4.3 Hz, 2H), 1.61-1.53 (m, 2H), 1.43 (s, 9H), 1.06 (dt, J=7.1, 2.3 Hz, 4H).

### Intermediate B6-tert-Butyl 4-(4-methyl-1-(3-methyloxetan-3-yl)-1H-pyrazol-5-yl)piperidine-1-carboxylate

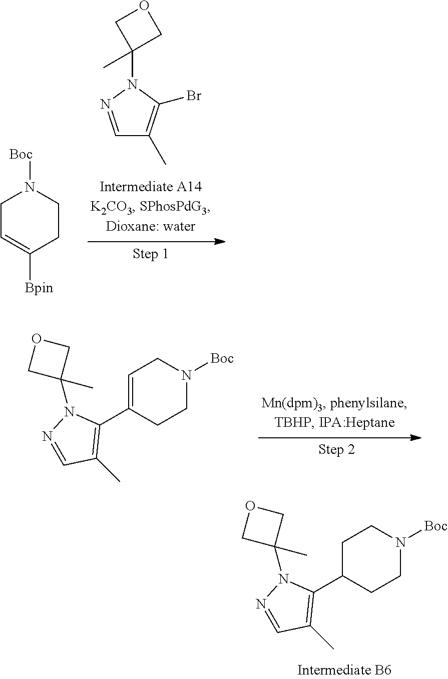

Step 1. tert-Butyl 4-(4-methyl-1-(3-methyloxetan-3-yl)-1H-pyrazol-5-yl)-3,6-dihydropyridine-1 (2H)-carboxylate. To a stirred solution of Intermediate A14 (198 g, 857 mmol) in 1,4-dioxane (3168 mL) and water (792 mL) was added tert-butyl 4-(4,4,5,5-tetramethyl-1,3,2-dioxaborolan-2-yl)-3,6-dihydropyridine-1 (2H)-carboxylate (344 g, 1114 mmol) at rt. The reaction mixture was degassed and purged with N₂ for 10 min, and K₂CO₃ (355 g, 2570 mmol) and SPhos Pd G3 (66.9 g, 86 mmol) were added. The reaction mixture was stirred at 90° C. for 6 h. The reaction mixture was quenched with water (2000 mL) and extracted with EtOAc (2×2000 mL). The combined organic extracts were dried (Na₂SO₄), filtered, concentrated under reduced pressure, and purified by chromatography, eluting with a gradient of 30-60% EtOAc in hexanes to give tert-butyl 4-(4-methyl-1-(3-methyloxetan-3-yl)-1H-pyrazol-5-yl)-3,6-dihydropyridine-1 (2H)-carboxylate (270 g, 95% yield), m/z (ESI): 334.2 (M+H)⁺.

Step 2. tert-Butyl 4-(4-methyl-1-(3-methyloxetan-3-yl)-1H-pyrazol-5-yl)piperidine-1-carboxylate, B6. To a stirred solution of tert-butyl 4-(4-methyl-1-(3-methyloxetan-3-yl)-1H-pyrazol-5-yl)-3,6-dihydropyridine-1 (2H)-carboxylate (24.0 g, 72.0 mmol) in IPA (168 mL) and n-heptane (168 mL) were added tris(2,2,6,6-tetramethyl-3,5-heptanedionato) manganese (III) (21.76 g, 36.0 mmol) and phenylsilane (13.43 mL, 108 mmol) in stepwise manner at 0° C. under N₂ atmosphere. The reaction mixture was stirred for 15 min at 0° C., and TBHP (5 M in nonane, 21.59 mL, 108 mmol) was added dropwise at 0° C. The reaction mixture was stirred at rt for 16 h. The reaction mixture was diluted with ice cold water (500 mL) followed by satd. aq. sodium metabisulfite solution (500 mL) and extracted with EtOAc (2×800 mL). The combined organic extracts were washed with brine (3×1000 mL), dried (Na₂SO₄), filtered, and concentrated. The crude material was purified by chromatography (elution: 0 to 30% EtOAc in hexanes), triturated with hexanes (200 mL), filtered, and dried to give Intermediate B6 (11 g. 46% yield), m/z (ESI): 336.4 (M+H)⁺. ¹H NMR (400 MHz, DMSO-d₆): δ (ppm) 7.18 (s, 1H). 5.04 (d, J=6.2 Hz, 2H), 4.5-4.6 (m, 2H), 4.01 (d, J=13.1 Hz, 2H), 2.79 (br s, 2H), 2.28 (tt, J=11.5, 4.3 Hz, 1H), 2.00 (s, 3H), 1.67-1.79 (m, 7H), 1.42 (s, 9H).

Intermediates in Table 1-6 were prepared following the procedures described for Intermediates B4, B5, or B6, using appropriate starting materials. All starting materials are commercially available or are described above.

TABLE 1-6

| | Chemical Structure & Name | LCMS: (ESI + ve ion) m/z; NMR | Comments |
|---|---|---|---|
| B7 | 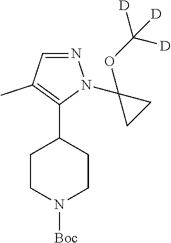 [SMILES: [2H]C([2H])([2H])OC1(n2ncc(C)c2C2CCN(C(=O)OC(C)(C)C)CC2)CC1] | m/z (ESI): 339.1 (M + H)⁺. | Intermediate A11 was used |
| | tert-butyl 4-(1-(1-(methoxy- | | |
| | d3)cyclopropyl)-4-methyl- | | |
| | 1H-pyrazol-5-yl)piperidine- | | |
| | 1-carboxylate | | |
| B8 | 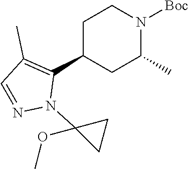 [SMILES: COC1(n2ncc(C)c2[C@H]2CCN(C(=O)OC(C)(C)C)[C@H](C)C2)CC1] | m/z (ESI): 350.0 (M + H)⁺. | Interemdiate A10 was used |
| | tert-butyl (2R,4S)-4-(1-(1- | | |
| | methoxycyclopropyl)-4- | | |
| | methyl-1H-pyrazol-5-yl)-2- | | |
| | methylpiperidine-1- | | |
| | carboxylate | | |
| B9 | 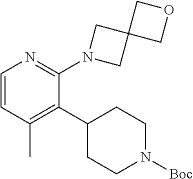 [SMILES: Cc1ccnc(N2CC3(COC3)C2)c1C1CCN(C(=O)OC(C)(C)C)CC1] | m/z (ESI): 374.0 (M + H)⁺. | Intermediate A17 was used |
| | tert-butyl 4-(4-methyl-2-(2- | | |
| | oxa-6-azaspiro[3.3]heptan-6- | | |
| | yl)pyridin-3-yl)piperidine-1- | | |
| | carboxylate | | |
| B10 | 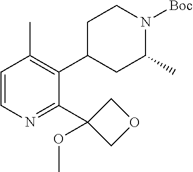 [SMILES: COC1(c2nccc(C)c2C2CCN(C(=O)OC(C)(C)C)[C@H](C)C2)COC1] | m/z (ESI): 377.3 (M + H)^{+ 1}H NMR (400 MHz, DMSO-d₆): δ (ppm) 8.28 (d, J = 4.9 Hz, 1H), 7.18 (d, J = 5.0 Hz, H), 5.14 (d, J = 32.4 Hz, 2H), 4.80 (dd, J = 18.4, 7.1 Hz, 2H), 4.40 (d, J = 27.1 Hz, 1H), 3.92 (dd, J = 24.6, 13.9 Hz, 1H), 2.98- 2.75 (m, 5H), 2.44 (s, 2H), 2.34- 2.05 (m, 1H), 1.86-2.01 (m, 1H), 1.68-1.39 (m, 11H), 1.14 (d, J = 18.9 Hz, 4H). | The sample was purified via Chiralpak IC (150 × 50) mm, 5μ, column with a mobile phase of liquid CO₂:MeOH (80:20) using a flowrate of 200 mL/min. 2^{nd} eluting isomer |
| | tert-butyl (2R,4S)-4-(2-(3- | | Stereochemistry was |
| | methoxyoxetan-3-yl)-4- | | arbitrarily assigned |
| | methylpyridin-3-yl)-2- | | |
| | methylpiperidine-1- | | |
| | carboxylate | | |
| B11 | 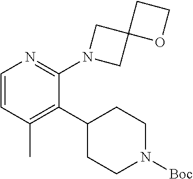 [SMILES: Cc1ccnc(N2CC3(CCO3)C2)c1C1CCN(C(=O)OC(C)(C)C)CC1] | m/z (ESI): 374.2 (M + H)⁺. | Intermediate A6 was used |
| | tert-butyl 4-(4-methyl-2-(1- | | |
| | oxa-6-azaspiro[3.3]heptan-6- | | |
| | yl)pyridin-3-yl)piperidine-1- | | |
| | carboxylate | | |
| B12 | 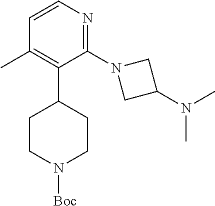 | m/z (ESI): 375.3 (M + H)⁺. | Intermediate A7 was used |
| | tert-butyl 4-(2-(3- | | |
| | (dimethylamino)azetidin-1- | | |
| | yl)-4-methylpyridin-3- | | |
| | yl)piperidine-1-carboxylate | | |
| B13 | 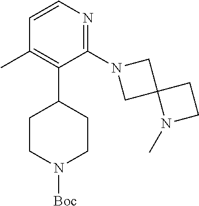 [SMILES: Cc1ccnc(N2CC3(CCN3C)C2)c1C1CCN(C(=O)OC(C)(C)C)CC1] | m/z (ESI): 387.1 (M + H)⁺. | Intermediate A8 was used |
| | tert-butyl 4-(4-methyl-2-(1- | | |
| | methyl-1,6- | | |
| | diazaspiro[3.3]heptan-6- | | |
| | yl)pyridin-3-yl)piperidine-1- | | |
| | carboxylate | | |
| B14 | 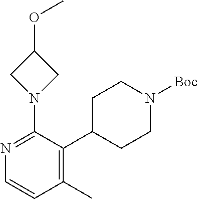 [SMILES: COC1CN(c2nccc(C)c2C2CCN(C(=O)OC(C)(C)C)CC2)C1] | m/z (ESI): 362.2 (M + H)⁺. | Intermediate A9 was used |
| | tert-butyl 4-(2-(3- | | |
| | methoxyazetidin-1-yl)-4- | | |
| | methylpyridin-3- | | |
| | yl)piperidine-1-carboxylate | | |
| B15 | 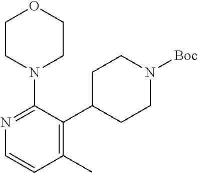 [SMILES: Cc1ccnc(N2CCOCC2)c1C1CCN(C(=O)OC(C)(C)C)CC1] | m/z (ESI): 362.2 (M + H)⁺. | Intermediate A21 was used |
| | tert-butyl 4-(4-methyl-2- | | |
| | morpholinopyridin-3- | | |
| | yl)piperidine-1-carboxylate | | |
| B16 | 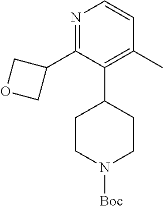 [SMILES: Cc1ccnc(C2COC2)c1C1CCN(C(=O)OC(C)(C)C)CC1] | m/z (ESI): 333.2 (M + H)⁺. | Intermediate A18 was used |
| | tert-butyl 4-(4-methyl-2- | | |
| | (oxetan-3-yl)pyridin-3- | | |
| | yl)piperidine-1-carboxylate | | |

### Intermediate B17-tert-Butyl(2R,4S)-2-methyl-4-(4-methyl-1-(oxetan-3-yl)-1H-pyrazol-5-yl)piperidine-1-carboxylate

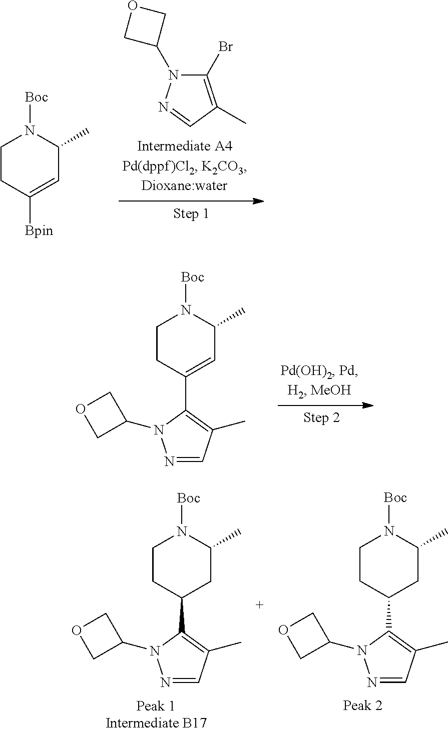

Step 1. tert-Butyl(R)-6-methyl-4-(4-methyl-1-(oxetan-3-yl)-1H-pyrazol-5-yl)-3,6-dihydropyridine-1 (2H)-carboxylate. To a degassed solution of tert-butyl 4-(4,4,5,5-tetramethyl-1,3,2-dioxaborolan-2-yl)-3,6-dihydropyridine-1 (2H)-carboxylate (13.40 g, 41.5 mmol), Intermediate A4 (7.5 g, 34.6 mmol), and K₂CO₃ (14.33 g, 104 mmol) in 1,4-dioxane (120 mL) and water (30.0 mL) was added Pd(dppf)Cl₂-DCM (1.41 g, 1.73 mmol), and the reaction mixture was heated to 90° C. for 16 h. The reaction was quenched with water (100 mL) and extracted with EtOAc (2×100 mL). The combined organic extracts were concentrated, and the crude residue was purified by column chromatography, eluting with a gradient of 25-30% EtOAc in pet. ether to provide tert-butyl(R)-6-methyl-4-(4-methyl-1-(oxetan-3-yl)-1H-pyrazol-5-yl)-3,6˜dihydropyridine-1 (2H)-carboxylate (11 g, 33.0 mmol, 95% yield), m/z (ESI): 334.3 (M+H)⁺.

Step 2. tert-Butyl(2R,4R)-2-methyl-4-(4-methyl-1-(oxetan-3-yl)-1H-pyrazol-5-yl)piperidine-1-carboxylate, Intermediate B17. To a stirred solution of tert-butyl(R)-6-methyl-4-(4-methyl-1-(oxetan-3-yl)-1H-pyrazol-5-yl)-3,6-dihydropyridine-1 (2H)-carboxylate (7 g, 20.99 mmol) in MeOH (140 mL) was added 10% weight palladium (II) hydroxide (5.90 g, 4.20 mmol). The reaction mixture was degassed thoroughly and stirred under H₂ bladder pressure for 16 h at 25° C. The reaction mixture was filtered through celite pad and washed with MeOH. The filtrate was concentrated and purified by chromatography eluting with a gradient of 20-30% EtOAc in pet. ether to give tert-butyl(2R)-2-methyl-4-(4-methyl-1-(oxetan-3-yl)-1H-pyrazol-5-yl)piperidine-1-carboxylate.

The stereoisomeric mixture containing tert-butyl(2R)-2-methyl-4-(4-methyl-1-(oxetan-3-yl)-1H-pyrazol-5-yl)piperidine-1-carboxylate was purified by Chiralpak IG (250×50) mm, 5 u, column with a mobile phase of liquid CO₂: [MeOH:ACN(1:1)] (80:20) with a flowrate of 180 mL/min to obtain a 1st eluting isomer and a 2^{nd} eluting isomer. The absolute stereochemistry of the isomers was determined to be ter-butyl(2R,4R)-2-methyl-4-(4-methyl-1-(oxetan-3-yl)-1H-pyrazol-5-yl)piperidine-1-carboxylate, Intermediate B17 as the 1st eluting isomer and tert-butyl(2R,4S)-2-methy 1-4-(4-methyl-1-(oxetan-3-yl)-1H-pyrazol-5-yl)piperidine-1-carboxylate as the 2^{nd} eluting isomer. 1st Eluting isomer: m/z (ESI); 336.3 (M+H)⁺. 2ªd Eluting isomer: m/z (ESD): 336.2 (M+H)⁺.

### Intermediate B18-tert-Butyl(2R,4S)-4-(1-(2-methoxyethyl)-4-methyl-1H-pyrazol-5-yl)-2-methylpiperidine-1-carboxylate

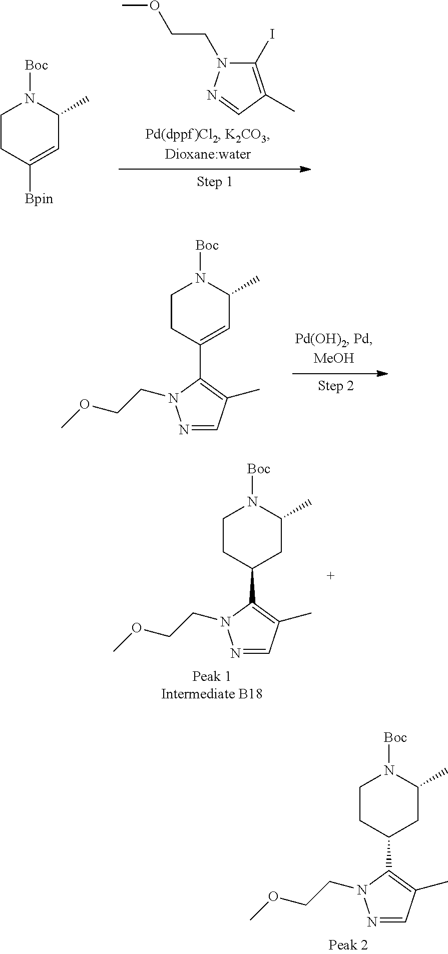

Step 1. tert-Butyl(R)-4-(1-(2-methoxyethyl)-4-methyl-1H-pyrazol-5-yl)-6-methyl-3,6-dihydropyridine-1 (2H)-carboxylate. To a solution of tert-butyl 4-(4,4,5,5-tetramethyl-1,3,2-dioxaborolan-2-yl)-3,6-dihydropyridine-1 (2H)-carboxylate (17.49 g, 54.1 mmol) in 1,4-dioxane (192 mL) and water (48.0 mL) were added Intermediate A1 (12 g, 45.1 mmol) and K₂CO₃ (18.70 g, 135 mmol) at rt. The reaction mixture was degassed with N₂ for 10 min followed by the addition of Pd(dppf)Cl₂-DCM (1.841 g, 2.255 mmol). The reaction mixture was heated to 90° C. for 16 h. The reaction mixture was quenched with ice cold water (500 mL) and extracted with EtOAc (3×400 mL). The combined organic extracts were dried, filtered, and concentrated. The crude residue was purified by column chromatography, using a gradient of 20-40% EtOAc in pet. ether to give tert-butyl(R)-4-(1-(2-methoxyethyl)-4-methyl-1H-pyrazol-5-yl)-6-methyl-3,6-dihydropyridine-1 (2H)-carboxylate (12 g. 35.8 mmol, 79% yield), m/z (ESI): 336.3 (M+H)⁺.

Step 2. (2R,4S)-4-(1-(2-Methoxyethyl)-4-methyl-1H-pyrazol-5-yl)-2-methylpiperidine-1-carboxylate, Intermediate B18. A solution of tert-butyl(R)-4-(1-(2-methoxyethyl)-4-methyl-1H-pyrazol-5-yl)-6-methyl-3,6-dihydropyridine-1 (2H)-carboxylate (11 g, 32.8 mmol) in MeOH (110 mL) was degassed with N₂ for 2 minutes. To the reaction mixture was added, 50 wt % palladium on carbon (3.49 g, 32.8 mmol) and 20 wt % palladium (II) hydroxide (4.61 g, 32.8 mmol). The reaction mixture was stirred at 50° C. under H₂ pressure (10 kg) for 16 h. The reaction mixture was filtered, washed with MeOH (200 mL), and concentrated to give a crude mixture of isomers (11 g), m/z (ESI): 338.3 (M+H)⁺.

A stereoisomeric mixture containing tert-butyl(2R)-4-(1-(2-methoxyethyl)-4-methyl-1H-pyrazol-5-yl)-2-methylpiperidine-1-carboxylate was purified by SFC with a LUX-C4 (250×50) mm, 5 u, column with a mobile phase of liquid CO₂:[MeOH:ACN(1:1)] (85:15) with a flowrate of 200 mL/min to obtain a 1^{st} eluting isomer and a 2^{nd} eluting isomer. The absolute stereochemistry of the isomers was assigned to be Intermediate B18 as the 18 eluting isomer and tert-butyl(2R,4R)-4-(1-(2-methoxyethyl)-4-methyl-1H-pyrazol-5-yl)-2-methylpiperidine-1-carboxylate as the 2^{nd} eluting isomer. 1^{st} Eluting isomer: m/z (ESI): 338.3 (M+H)⁺. 2^{nd} Eluting isomer: m/z (ESI): 338.1 (M+H)⁺,

### Intermediate B19-tert-Butyl(2R,4S)-2-methyl-4-(4-methyl-1-((S)-tetrahydrofuran-3-yl)-1H-pyrazol-5-yl)piperidine-1-carboxylate

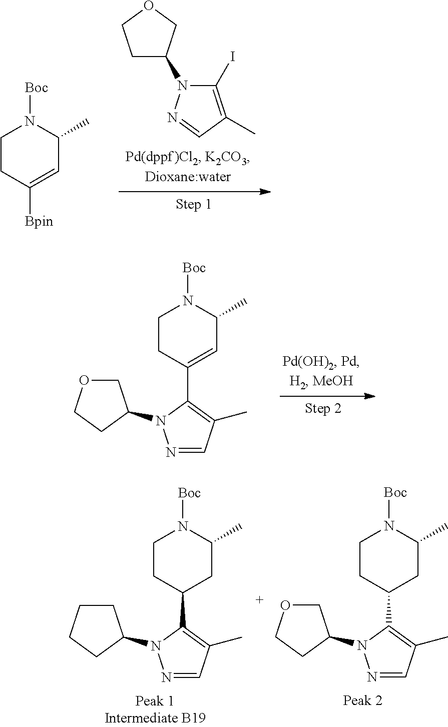

Step 1. tert-Butyl(R)-6-methyl-4-(4-methyl-1-((S)-tetrahydrofuran-3-yl)-1H-pyrazol-5-yl)-3,6-dihydropyridine-1 (2H)-carboxylate. To a solution of tert-butyl 4-(4,4,5,5-tetramethyl-1,3,2-dioxaborolan-2-yl)-3,6-dihydropyridine-1 (2H)-carboxylate (6.97 g, 21.6 mmol) in 1,4-dioxane (100 mL) and water (10 mL) were added Intermediate A12 (5 g. 17.98 mmol) and KaPO₄ (11.45 g, 53.9 mmol) at rt. The reaction mixture was degassed with N₂ for 10 min followed by the addition of Pd(dppf)Cl₂-DCM (1.47 g, 1.80 mmol). The reaction mixture was heated to 100° C. for 4 h. The reaction mixture was quenched in ice cold water (500 mL) and extracted with EtOAc (3×400 mL). The combined organic extracts were dried over Na₂SO₄, filtered, and concentrated. The crude residue was purified by column chromatography using a gradient of 25-50% EtOAc in hexanes to give tert-butyl (R)-6-methyl-4-(4-methyl-1-((S)-tetrahydrofuran-3-yl)-1H-pyrazol-5-yl)-3,6-dihydropyridine-1 (2H)-carboxylate (6 g, 17.3 mmol, 96% yield), m/z (ESI): 348.1 (M+H)⁺.

Step 2. tert-Butyl(2R)-2-methyl-4-(4-methyl-1-((S)-tetrahydrofuran-3-yl) ˜1H-pyrazol-5-yl)piperidine-1-carboxylate. To a degassed (N₂) solution of ter-butyl(R)-6-methyl-4-(4-methyl-1-((S)-tetrahydrofuran-3-yl)-1H-pyrazol-5-yl)-3,6-dihydropyridine-1 (2H)-carboxylate (5.5 g, 15.83 mmol) in MeOH (83 mL) was added 50 wt % palladium on activated carbon (2.5 g, 2.35 mmol) and 20 wt % palladium (II) hydroxide on carbon (2.5 g, 17.80 mmol). The reaction mixture was degassed and stirred under H₂ atmosphere (60 psi) for 5 days at rt. The reaction mixture was filtered through a pad of celite and washed with MeOH (200 mL). The filtrate was concentrated and purified by column chromatography, eluting with 15% EtOAc in pet. ether to provide tert-butyl(2R)-2-methyl-4-(4-methyl-1-((S)-tetrahydrofuran-3-yl)-1H-pyrazol-5-yl)piperidine-1-carboxylate (5 g, 14.31 mmol, 90% yield), m/z (ESI): 350.1 (M+H)⁺.

The stereoisomeric mixture containing tert-butyl(2R)-2-methyl-4-(4-methyl-1-((S)-tetrahydrofuran-3-yl)-1H-pyrazol-5-yl)piperidine-1-carboxylate (4.8 g, 13.73 mmol) was purified by SFC (Chiralpak IC-H, 150×50 mm 5 μm column with a mobile phase of IPA: liquid CO₂ (30:70) using a flowrate of 180 mL/min), to obtain a 1^{st} eluting isomer and a 2^{nd} eluting isomer. The stereochemistry of the isomers was assigned arbitrarily to be tert-butyl(2R,4R)-2-methyl-4-(4-methyl-1-((S)-tetrahydrofuran-3-yl)-1/pyrazol-5-yl)piperidine-1-carboxylate, Intermediate B19 (1.3 g, 3.72 mmol, 27% yield) as the 1st eluting isomer and tert-buty 1 (2R,4S)-2-methyl-4-(4-methyl-1-((S)-tetrahydrofuran-3-yl)-1H-pyrazol-5-yl)piperidine-1-carboxylate as the 2^{nd} eluting isomer. 1^{st} Eluting isomer: m/z (ESI): 350.3 (M+H)⁺. 2^{nd} Eluting isomer: m/z (ESI): 350.3 (M+H)⁺.

Intermediates in Table 1-7 were prepared following the procedures described for Intermediates B17, B18, and B19 using appropriate starting materials. All starting materials are commercially available or are described above.

TABLE 1-7

| | Chemical Structure & Name | LCMS: (ESI + ve ion) m/z; NMR | Comments |
|---|---|---|---|
| B20 | 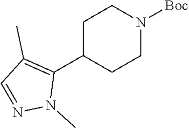 [SMILES: Cc1cnn(C)c1C1CCN(C(=O)OC(C)(C)C)CC1] | m/z (ESI): 280.2 (M + H)⁺. | |
| | tert-butyl 4-(1,4-dimethyl- | | |
| | 1H-pyrazol-5-yl)piperidine- | | |
| | 1-carboxylate | | |
| B21 | 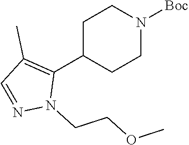 [SMILES: COCCn1ncc(C)c1C1CCN(C(=O)OC(C)(C)C)CC1] | m/z (ESI): 324.2 (M + H)⁺. ¹H NMR (400 MHz, DMSO-d₆) δ (ppm) 7.12 (s, 1H), 4.20 (t, 2H), J = 5.4 Hz), 3.9-4.2 (m, 2H), 3.59 (t, 2H, J = 5.4 Hz), 3.20 (s, 3H), 3.00 (ddt, 1H, J = 16.0, 10.9, 4.9 Hz), 2.79 (br s, 2H), 2.00 (s, 3H), 1.6-1.8 (m, 4H), 1.42 (s, 9H). | Intermediate A1 was used |
| | tert-butyl 4-(1-(2- | | |
| | methoxyethyl)-4-methyl-1H- | | |
| | pyrazol-5-yl)piperidine-1- | | |
| | carboxylate | | |
| B22 | 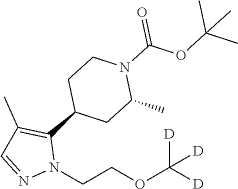 [SMILES: [2H]C([2H])([2H])OCCn1ncc(C)c1[C@H]1CCN(C(=O)OC(C)(C)C)[C@H](C)C1] | m/z (ESI): 341.1 (M + H)⁺. ¹H NMR (400 MHz, DMSO-d₆) δ (ppm) 7.12 (s, 1H), 4.21-4.36 (m, 2H), 4.09-4.20 (m, 1H), 3.91 (d, J = 15.0 Hz, 1H), 3.52-3.65 (m, 2H), 3.20 (br s, 1H), 2.87-3.05 (m, 1H), 2.01 (s, 3H), 1.92 (dd, J = 13.1, 6.9 Hz, 1H), 1.66 (s, 2H), 1.50- 1.58 (m, 1H), 1.42 (s, 9H), 1.18 (td, J = 7.2, 3.5 Hz, 3H). | Intermediate B18 was used, and specifically demethylated, protected, and alkylated. |
| | tert-butyl (2R,4S)-4-(1-(2- | | |
| | (methoxy-d3)ethyl)-4- | | |
| | methyl-1H-pyrazol,-5-yl)-2- | | |
| | methylpiperidine-1- | | |
| | carboxylate | | |
| B23 | 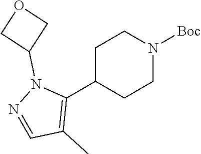 [SMILES: Cc1cnn(C2COC2)c1C1CCN(C(=O)OC(C)(C)C)CC1] | m/z (ESI): 322.2 (M + H)⁺. | Intermediate A4 was used |
| | tert-butyl 4-(4-methyl-1- | | |
| | (oxetan-3-yl)-1H-pyrazol-5- | | |
| | yl)piperidine-1-carboxylate | | |
| B24 | 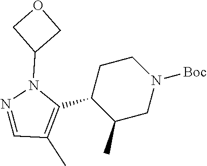 [SMILES: Cc1cnn(C2COC2)c1[C@@H]1CCN(C(=O)OC(C)(C)C)C[C@H]1C] | m/z (ESI): 336.1 (M + H)⁺. | Intermediate A4 was used |
| | tert-butyl (3S,4R)-3-methyl- | | |
| | 4-(4-methyl-1-(oxetan-3-yl)- | | |
| | 1H-pyrazol-5-yl)piperidine- | | |
| | 1-carboxylate | | |
| B25 | 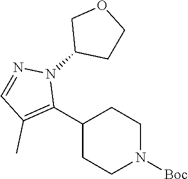 [SMILES: Cc1cnn([C@H]2CCOC2)c1C1CCN(C(=O)OC(C)(C)C)CC1] | m/z (ESI): 336.1 (M + H)⁺. | Intermediate A12 was used |
| | tert-butyl (S)-4-(4-methyl-1- | | |
| | (tetrahydrofuran-3-yl)-1H- | | |
| | pyrazol-5-yl)piperidine-1- | | |
| | carboxylate | | |
| B26 | 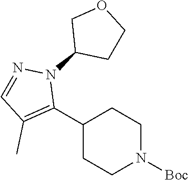 [SMILES: Cc1cnn([C@@H]2CCOC2)c1C1CCN(C(=O)OC(C)(C)C)CC1] | m/z (ESI): 336.1 (M + H)⁺. | Intermediate A12-1 was used |
| | tert-butyl (R)-4-(4-methyl-1- | | |
| | (tetrahydrofuran-3-yl)-1H- | | |
| | pyrazol-5-yl)piperidine-1- | | |
| | carboxylate | | |
| B27/B28 Mixture | 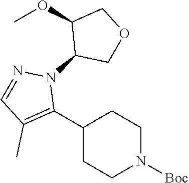 [SMILES: CO[C@H]1COC[C@H]1n1ncc(C)c1C1CCN(C(=O)OC(C)(C)C)CC1] | m/z (ESI): 366.3 (M + H)⁺. | Mixture of cis steroisomers was used |
| | tert-butyl 4-(1-((3R,4R)-4- | | |
| | methoxytetrahydrofuran-3- | | |
| | yl)-4-methyl-1H-pyrazol-5- | | |
| | yl)piperidine-1-carboxylate | | |
| | 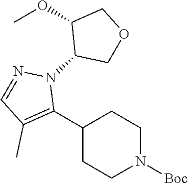 [SMILES: CO[C@@H]1COC[C@@H]1n1ncc(C)c1C1CCN(C(=O)OC(C)(C)C)CC1] | m/z (ESI): 366.3 (M + H)⁺. | |
| | tert-butyl 4-(1-((3S,4S)-4- | | |
| | methoxytetrahydrofuran-3- | | |
| | yl)-4-methyl-1H-pyrazol-5- | | |
| | yl)piperidine-1-carboxylate | | |
| B29/B30 Mixture | 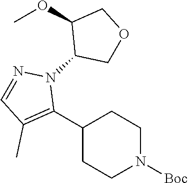 [SMILES: CO[C@H]1COC[C@@H]1n1ncc(C)c1C1CCN(C(=O)OC(C)(C)C)CC1] | m/z (ESI): 366.3 (M + H)⁺. | Mixture of trans stereoisomers was used |
| | tert-butyl 4-(1-((3S,4R)-4- | | |
| | methoxytetrahydrofuran-3- | | |
| | yl)-4-methyl-1H-pyrazol-5- | | |
| | yl)piperidine-1-carboxylate | | |
| | 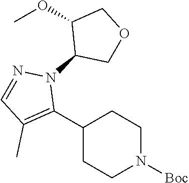 [SMILES: CO[C@@H]1COC[C@H]1n1ncc(C)c1C1CCN(C(=O)OC(C)(C)C)CC1] | m/z (ESI): 366.3 (M + H)⁺. | |
| | tert-butyl 4-(1-((3R,4S)-4- | | |
| | methoxytetrahydrofuran-3- | | |
| | yl)-4-methyl-1H-pyrazol-5- | | |
| | yl)piperidine-1-carboxylate | | |
| B31 | 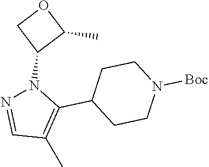 [SMILES: Cc1cnn([C@@H]2CO[C@@H]2C)c1C1CCN(C(=O)OC(C)(C)C)CC1] | m/z (ESI): 336.0 (M + H)⁺. | Intermediate A3 was used; SFC was performed with a Chiralpak IC, 150 × 50 mm, 5 μm, column with a mobile phase of 20% MeOH in liquid CO₂ using a flow rate of 180 mL/min (2^{nd} eluting isomer) |
| | tert-butyl 4-(4-methyl-1- | | Chirality was |
| | ((2S,3S)-2-methyloxetan-3- | | assigned arbitrarily |
| | yl)-1H-pyrazol-5- | | |
| | yl)piperidine-1-carboxylate | | |
| B31′ | 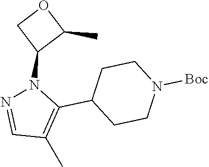 [SMILES: Cc1cnn([C@H]2CO[C@H]2C)c1C1CCN(C(=O)OC(C)(C)C)CC1] | m/z (ESI): 336.0 (M + H)⁺. | |
| | tert-butyl 4-(4-methyl-1- | | |
| | ((2S,3S)-2-methyloxetan-3- | | |
| | yl)-1H-pyrazol-5- | | |
| | yl)piperidine-1-carboxylate | | |
| B32 | 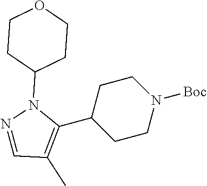 [SMILES: Cc1cnn(C2CCOCC2)c1C1CCN(C(=O)OC(C)(C)C)CC1] | m/z (ESI): 350.0 (M + H)⁺. | Intermediate A2 was used; |
| | tert-butyl 4-(4-methyl-1- | | |
| | (tetrahydro-2H-pyran-4-yl)- | | |
| | 1H-pyrazol-5-yl)piperidine- | | |
| | 1-carboxylate | | |
| B33 | 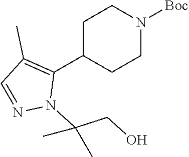 [SMILES: Cc1cnn(C(C)(C)CO)c1C1CCN(C(=O)OC(C)(C)C)CC1] | m/z (ESI): 338.1 (M + H)⁺. | Intermediate A16 was used; |
| | tert-butyl 4-(1-(1-hydroxy-2- | | |
| | methylpropan-2-yl)-4- | | |
| | methyl-1H-pyrazol-5-yl)- | | |
| | 3,6-dihydropyridine-1(2H)- | | |
| | carboxylate | | |
| B34 | 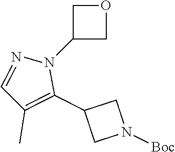 [SMILES: Cc1cnn(C2COC2)c1C1CN(C(=O)OC(C)(C)C)C1] | m/z (ESI): 294.2 (M + H)⁺. | Intermediate A4 was used. |
| | tert-butyl 3-(4-methyl-1- | | |
| | (oxetan-3-yl)-1H-pyrazol-5- | | |
| | yl)azetidine-1-carboxylate | | |
| B35 | 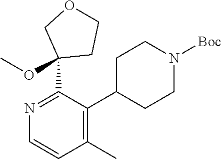 | m/z (ESI): 391.1 (M + H)⁺. | The racemic mixturee was separated via SFC using a ChiralPak IC, 2 × 25 cm 5 μm column with a mobile phase of 20% iPrOH using a flowrate of 120 mL/min. |
| | tert-butyl (S)-4-(2-(3- | | 2^{nd} eluting isomer |
| | methoxytetrahydrofuran-3- | | The stereochemistry |
| | yl)-4-methylpyridin-3- | | was assigned |
| | yl)piperidine-1-carboxylate | | arbitrarily. |
| B36 | 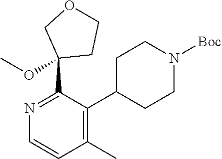 | m/z (ESI): 391.1 (M + H)⁺. | The racemic mixture was separated via SFC using a ChiralPak IC, 2 × 25 cm 5 μm column with a mobile phase of 20% iPrOH using a flowrate of 120 mL/min. 1^{st} eluting isomer |
| | tert-butyl (R)-4-(2-(3- | | The stereochemistry |
| | methoxytetrahydrofuran-3- | | was assigned |
| | yl)-4-methylpyridin-3- | | arbitrarily. |
| | yl)piperidine-1-carboxylate | | |
| B37 | 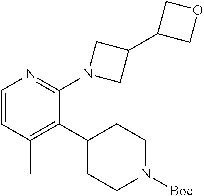 [SMILES: Cc1ccnc(N2CC(C3COC3)C2)c1C1CCN(C(=O)OC(C)(C)C)CC1] | m/z (ESI): 388.2 (M + H)⁺. | Intermediate A5 was used |
| | tert-butyl 4-(4-methyl-2-(3- | | |
| | (oxetan-3-yl)azetidin-1- | | |
| | yl)pyridin-3-yl)piperidine-1- | | |
| | carboxylate | | |
| B38 | 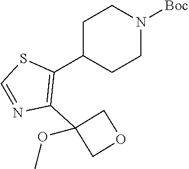 [SMILES: COC1(c2ncsc2C2CCN(C(=O)OC(C)(C)C)CC2)COC1] | m/z (ESI): 355.3 (M + H)⁺ | Made using 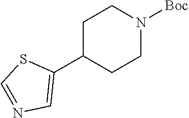 [SMILES: CC(C)(C)OC(=O)N1CCC(c2cncs2)CC1] |
| | tert-butyl 4-(4-(3- | | |
| | methoxyoxetan-3-yl)thiazol- | | |
| | 5-yl)piperidine-1- | | |
| | carboxylate | | |

### Intermediate B39-2-(3-Methoxyoxetan-3-yl)-4-methyl-3-((3S,4R)-3-methylpiperidin-4-yl)pyridine

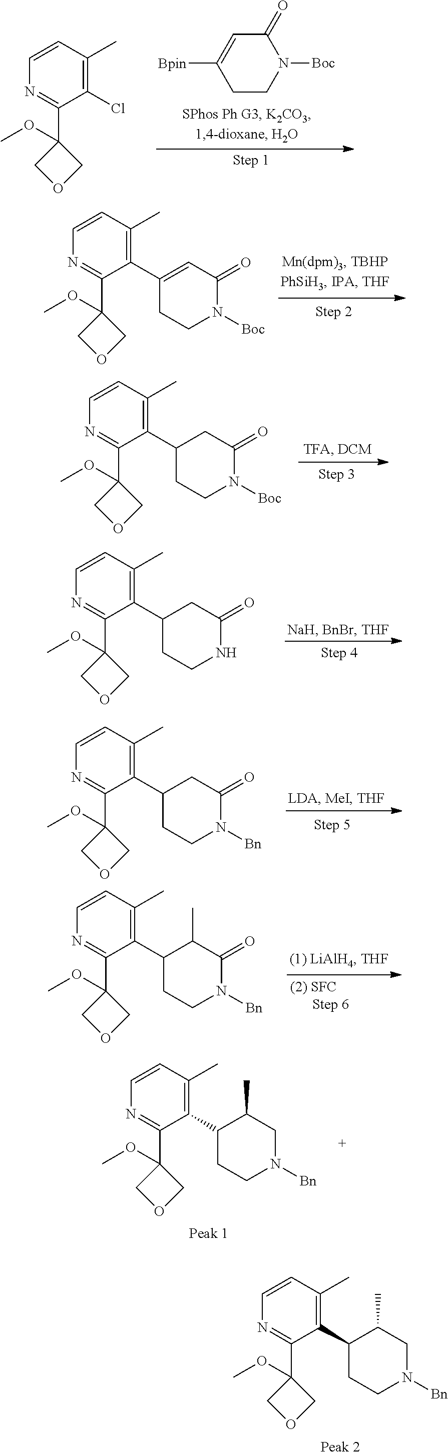

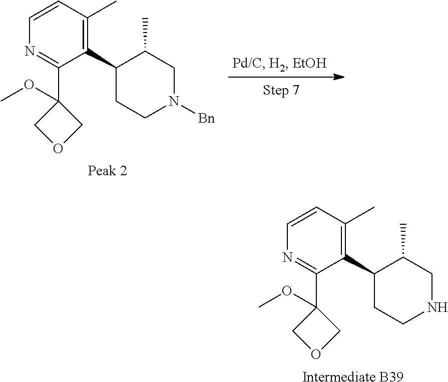

Step 1. tert-Butyl 2-(3-methoxyoxetan-3-yl)-4-methyl-6′-oxo-3′,6′-dihydro-[3,4′-bipyridine]-1′(2′H)-carboxylate. A mixture of 3-chloro-2-(3-methoxyoxetan-3-yl)-4-methylpyridine (10 g, 46.8 mmol), tert-butyl 6-oxo-4-(4,4,5,5-tetramethyl-1,3,2-dioxaborolan-2-yl)-3,6-dihydropyridine-1 (2H)-carboxylate (27.2 g, 84 mmol), and K₂CO₃ (22.64 g, 164 mmol) in 1,4-dioxane (115 mL) and water (26 mL) was purged with N₂ for 10 min. Then, SPhos Pd G3 (1.83 g, 2.34 mmol) was added and purged with N₂ for 2 min. The reaction mixture was stirred at 110° C. for 16 h, cooled to rt, water (200 mL) was added, and extracted with EtOAc (2×500 mL). The combined organic extracts were dried over Na₂SO₄, filtered, and concentrated in vacuo. The crude material was purified by chromatography, eluting with a gradient of 60-70% EtOAc in hexanes to provide tert-butyl 2-(3-methoxyoxetan-3-yl)-4-methyl-6′-oxo-3′,6′-dihydro-[3,4′-bipyridine]-1′(2′H)-carboxylate.

Step 2. tert-Butyl 4-(2-(3-methoxyoxetan-3-yl)-4-methylpyridin-3-yl)-2-oxopiperidine-1-carboxylate. To a stirred solution of tert-butyl 2-(3-methoxyoxetan-3-yl)-4-methyl-6′-oxo-3′,6′-dihydro-[3,4′-bipyridine]-1′(2′H)-carboxylate (9.5 g, 25.4 mmol) in IPA (190 mL) and THF (190 mL) at 0° C. under N₂ atmosphere was added Mn(dpm)₃ (7.67 g. 12.69 mmol) and phenylsilane (4.69 mL, 38.1 mmol) followed by TBHP (70% in water) (6.95 mL, 50.7 mmol). The reaction mixture was stirred at 0° C. for 30 min, then at 25° C. for 16 h. The reaction mixture was cooled and quenched with water (300 mL) and extracted with EtOAc (3×200 mL). The combined organic extracts were washed with water (100 mL), dried over Na₂SO₄, filtered, and concentrated under reduced pressure. The crude residue was purified by column chromatography (elution: 0-50% EtOAc in hexanes) to give tert-butyl 4-(2-(3-methoxyoxetan-3-yl)-4-methylpyridin-3-yl)-2-oxopiperidine-1-carboxylate, m/z (ESI): 377.2 (M+H) 1. ¹H NMR (400 MHz, DMSO-d₆): δ (ppm) 8.32 (d, J=4.9 Hz, 1H), 7.24 (d, J=5.0 Hz, 1H), 5.17 (d, J=7.0 Hz, 1H), 5.07 (d, J=7.1 Hz, 1H), 4.78 (dd, J=16.6, 7.0 Hz, 2H), 3.87 (dt, J=12.9, 4.6 Hz, 1H), 3.64-3.50 (m, 1H), 3.02 (dt, J=11.5, 6.1 Hz, 3H), 2.93 (s, 3H), 2.43 (s, 3H), 2.15 (t, J=10.8 Hz, 1H), 1.91 (dd, J=14.0, 5.0 Hz, 1H), 1.47 (s, 9H).

Step 3. 4-(2-(3-Methoxyoxetan-3-yl)-4-methylpyridin-3-yl)piperidin-2-one. To a solution of tert-butyl 4-(2-(3-methoxyoxetan-3-yl)-4-methylpyridin-3-yl)-2-oxopiperidine-1-carboxylate in DCM (40 mL) at 0° C. under N₂, was added TFA (4.0 mL, 53.10 mmol). The reaction mixture was stirred at rt for 2 h. The reaction mixture was quenched with satd. NaHCO₃ solution at 0° C., and extracted with 10% MeOH in DCM (4×60 mL). The combined organic extracts were over Na₂SO₄, filtered, and concentrated under reduced pressure to afford 4-(2-(3-methoxyoxetan-3-yl)-4-methylpyridin-3-yl)piperidin-2-one, which was directly taken to next step, m/z (ESI): 277.1 (M+H)⁺. 3H NMR (400 MHz, DMSO-d₆): δ (ppm) 8.30 (dd, J=9.9, 4.9 Hz, 1H), 7.67 (d, J=3.9 Hz, 1H), 7.21 (dd, J=8.8, 4.9 Hz, 1H), 5.22 (d, J=7.0 Hz, 1H), 5.01 (d, J=7.0 Hz, 1H), 4.79 (dd, J=7.0, 0.9 Hz, 1H), 4.74 (dd.]=7.0, 0.9 Hz, 1H), 3.59 (s, 3H), 3.28-3.09 (m, 1H), 2.93 (s, 3H), 2.58 (t, J=13.0 Hz, 2H), 2.44 (d, J=9.5 Hz, 2.33-2.09 (m, 1H), 1.75 (q, J=8.2, 6.5 Hz, 2H).

Step 4. 1-Benzyl-4-(2-(3-methoxyoxetan-3-yl)-4-methylpyridin-3-yl)piperidin-2-one. To a solution of 4-(2-(3-methoxyoxetan-3-yl)-4-methylpyridin-3-yl)piperidin-2-one (4.0 g, 14.48 mmol) in THF (60 mL) at 0° C. under N₂, was added NaH (60% in mineral oil) (1.26 g, 29.0 mmol). The reaction mixture was stirred at 0° C. for 30 min. Benzyl bromide (4.95 g, 29.0 mmol) was added at 0° C., and stirred for 2 h. The reaction mixture was quenched with satd. NH₄Cl solution (20 mL) and extracted with EtOAc (3×80 mL). The combined organic extracts were washed with water (50 mL), dried over Na₂SO₄, filtered, and concentrated under reduced pressure. The crude residue was purified by column chromatography (elution: 0-60% EtOAc in hexanes) to give 1-benzyl-4-(2-(3-methoxyoxetan-3-yl)-4-methylpyridin-3-yl)piperidin-2-one, m/z (ESI): 367.0 (M+H)⁺. ¹H NMR (400 MHz, DMSO-d₆): δ (ppm) 8.32 (d, J=4.9 Hz, 1H), 7.42-7.34 (m, 2H), 7.32-7.18 (m, 4H), 5.22 (d, J=7.1 Hz, 1H), 5.03 (d, J=7.0 Hz, 1H), 4.73 (dd, J=7.1, 4.5 Hz, 2H), 4.66 (d, J=14.9 Hz, 1H), 4.49 (d, J=14.9 Hz, 1H), 3.30-3.21 (m, 2H), 2.92 (s, 4H), 2.79 (dd, J=17.3, 12.0 Hz, 1H), 2.48-2.39 (m, 4H), 2.25 (h, J=6.4 Hz, 1H), 1.81 (d, J=13.2 Hz, 1H).

Stop 5. 1-Benzyl-4-(2-(3-methoxyoctan-3-yl)-4-methylpyridin-3-yl)-3-methylpiperidin-2-one. To a solution of 1-benzyl-4-(2-(3-methoxyoxetan-3-yl)-4-methylpyridin-3-yl)piperidin-2-one (670 mg, 1.83 mmol) in THF (13.40 mL) at −78° C. was added LDA (2 M in THE, 1.1 mL, 2.2 mmol) dropwise. After stirring for 30 min at −78° C., Mel (171 μL, 2.74 mmol) was added at −78° C., and the solution allowed to warm to rt and stirred for 2 h. The reaction was quenched with NH₄Cl while cooling in an ice bath. The layers were separated, and the aqueous layer was extracted with EtOAc (2×100 mL). The combined organic extracts were dried over Na₂SO₄ and concentrated. The crude was purified by reverse phase chromatography using ACN in water (35 to 40%), to provide 1-benzyl-4-(2-(3-methoxyoxetan-3-yl)-4-methylpyridin-3-yl)-3-methylpiperidin-2-one, m/z (ESI): 381.3 (M+H)⁺. ¹H NMR (400 MHz, DMSO-d₆): δ (ppm) 8.32 (d,)=4.9 Hz, 1H), 7.37 (t, J=7.4 Hz, 2H), 7.31-7.17 (m, 4H), 5.16 (d, J=7.2 Hz, 1H), 5.04 (d, J=6.9 Hz, 1H), 4.72 (d, J=7.2 Hz, 1H), 4.68-4.46 (m, 3H), 3.02 (dd,/=11.5, 7.0 Hz, 2H), 2.93 (s, 3H), 2.71 (d, J=3.6 Hz, 2H), 2.46 (s, 3H), 2.24 (s, 1H), 1.85 (d, J=10.4 Hz, 1H), 0.91 (d, J=7.1 Hz, 3H).

Step 6. 3-(1-Benzyl-3-methylpiperidin-4-yl)-2-(3-methoxyoxetan-3-yl)-4-methylpyridine. To a solution of 1-benzyl-4-(2-(3-methoxyoxetan-3-yl)-4-methylpyridin-3-yl)-3-methylpiperidin-2-one (530 mg, 1.39 mmol) in THF (31.8 mL) at 0° C. was added LAH (2 M in THE, 1.4 mL, 2.79 mmol). The reaction mixture was heated to 60° C., and stirred at 60° C. for 15 min. The reaction mixture was cooled to 0° C., and then satd. aq. Na₂SO₄ was added. The mixture was extracted with EtOAc (3×30 mL). The combined organic extracts were over Na₂SO₄, filtered, and concentrated. The crude was purified by reverse phase chromatography, eluting with a gradient of 20 to 30% ACN in water to provide 3-(1-benzyl-3-methylpiperidin-4-yl)-2-(3-methoxyoxetan-3-yl)-4-methylpyridine, m/z (ESI): 367.4 (M+H)⁺.

The above material was purified via SFC using a Chiralpak IG 250×20 mm, 5 μm column with a mobile phase of 1:1 ACN/MeOH in liquid CO₂ with a flow rate of 70 mL/min to provide a 1st eluting isomer: 3-((3R,4S)-1-benzyl-3-methylpiperidin-4-yl)-2-(3-methoxyoxetan-3-yl)-4-methylpyridine: 3H NMR (400 MHz, DMSO-d₆): δ (ppm) 8.27 (d, J=4.8 Hz, 1H), 7.34-7.24 (m, 5H), 7.18 (d, J=5.0 Hz, 1H), 5.18-4.99 (m, 2H), 4.84-4.59 (m, 2H), 3.48 (s, 2H), 2.98-2.85 (m, SH), 2.46 (s, 3H), 2.38-2.29 (m, 1H), 2.15-2.06 (m, 1H), 2.04-1.88 (m, 2H), 1.66-1.50 (m, 2H), 0.53 (d, J=6.7 Hz, 3H); and a 2ª4 eluting isomer: 3-((3S,4R)-1-benzyl-3-methylpiperidin-4-yl)-2-(3-methoxyoxetan-3-yl)-4-methylpyridine: 3H NMR (400 MHz, DMSO-d₆): δ (ppm) 8.27 (d, J=4.8 Hz, 1H), 7.48-7.20 (m, 5H), 7.18 (d, J=5.0 Hz, 1H), 5.18-4.99 (m, 2H), 4.84-4.59 (m, 2H), 3.48 (s, 2H), 2.97-2.85 (m, 5H), 2.46 (s, 3H), 2.39-2.30 (m, 1H), 2.18-2.08 (m, 1H), 2.04-1.88 (m, 2H), 1.66-1.50 (m, 2H), 0.53 (d, J=6.7 Hz, 3H).

Step 7. 2-(3-Methoxyoxetan-3-yl)-4-methyl-3-((3S,4R)-3-methylpiperidin-4-yl)pyridine. To a stirred solution of (peak 2)₃-((38,4R)-1-benzyl-3-methylpiperidin-4-yl)-2-(3-methoxyoxetan-3-yl)-4-methylpyridine (160 mg, 0.437 mmol) in EtOH (1 mL) was added 10 wt % palladium on activated carbon (167 mg, 0.157 mmol) under N₂ atmosphere and the reaction was continued to stir at 25° C. under H₂ bladder pressure for 1 h. The reaction mixture was filtered, and the filtrate was concentrated to provide crude Intermediate B39, m/z (ESI): 277.0 (M+H)⁺. ¹H NMR (400 MHz, DMSO-d₆): δ (ppm) 8.27 (d, J=4.9 Hz, 1H), 7.17 (d, J=4.8 Hz, 1H), 5.17-5.03 (m, 2H), 4.81-4.66 (m, 2H), 4.10 (d, J=5.4 Hz, 1H), 3.17 (d, J=4.5 Hz, 2H), 3.02 (1, J=10.2 Hz, 2H), 2.92 (d, J=2.6 Hz, 3H), 2.47 (s, 3H), 2.26-1.86 (m, 4H), 0.56 (dd, J=18.3, 5.7 Hz, 3H).

Intermediates in Table 1-8 were prepared following the procedure described for Intermediate B39 (prior to Bn deprotection), using appropriate starting materials. All starting materials are commercially available or are described above.

TABLE 1-8

| | Chemical Structure & Name | LCMS: (ESI + ve ion) m/z; NMR | Comments |
|---|---|---|---|
| B40 | 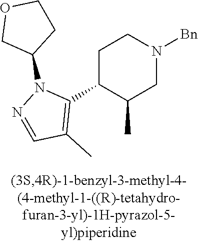 [SMILES: Cc1cnn([C@@H]2CCOC2)c1[C@@H]1CCN(Cc2ccccc2)C[C@H]1C] | m/z (ESI): 340.4 (M + H)⁺. ¹H NMR (400 MHz, DMSO-d6) δ 7.36-7.31 (m, 4H), 7.29-7.26 (m, 1H), 7.19 (s, 1H), 5.07-5.04 (m, 1H), 4.03-3.93 (m, 2H), 3.84-3.79 (m, 1H), 3.72- 3.69 (m, 1H), 3.50 (s, 2H), 2.89-2.87 (m, 2H), 2.47 (s, 1H), 2.26-2.18 (m, 2H), 2.06-2.01 (m, 5H), 1.83-1.72 (m, 2H), 1.69-1.59 (m, 1H), 0.59- 0.57 (m, 3H). | Intermediate A12 used; SFC was performed with a CHIRALCEL OJ-H (250 × 30) mm, 5 μm, column with a mobile phase of liquid CO₂: 0.2% DEA in MeOH: IPA (1:1)] (95:5) using a flowrate of 150 mL/min (1^{st} eluting peak) |
| B41 | 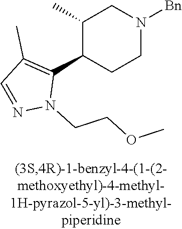 [SMILES: COCCn1ncc(C)c1[C@@H]1CCN(Cc2ccccc2)C[C@H]1C] | m/z (ESI): 328.0 (M + H)⁺. | Intermediate A1 was used; Sample was purified by SFC using a Chiral Pak AD-H (150 × 50) mm, 5 μm, column with a mobile phase of liquid CO₂: 0.5% IPAm in IPA (90:10), using a flow rate of 170 mL/min (1^{st} eluting isomer) |
| B42 | 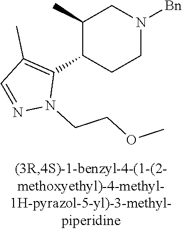 [SMILES: COCCn1ncc(C)c1[C@H]1CCN(Cc2ccccc2)C[C@@H]1C] | m/z (ESI): 328.0 (M + H)⁺. | Intermediate A1 was used, Sample was purified by SFC using a Chiral Pak AD-H (150 × 50) mm, 5 μm, column with a mobile phase of liquid CO₂: 0.5% IPAm in IPA (90:10), using a flow rate of 170 mL/min (2^{nd} eluting isomer) |

### Intermediate B43 and B44-tert-Butyl(R)-4-(3-(1-methoxyethyl)-5-methylpyridazin-4-yl)piperidine-1-carboxylate and tert-butyl(S)-4-(3-(1-methoxyethyl)-5-methylpyridazin-4-yl)piperidine-1-carboxylate

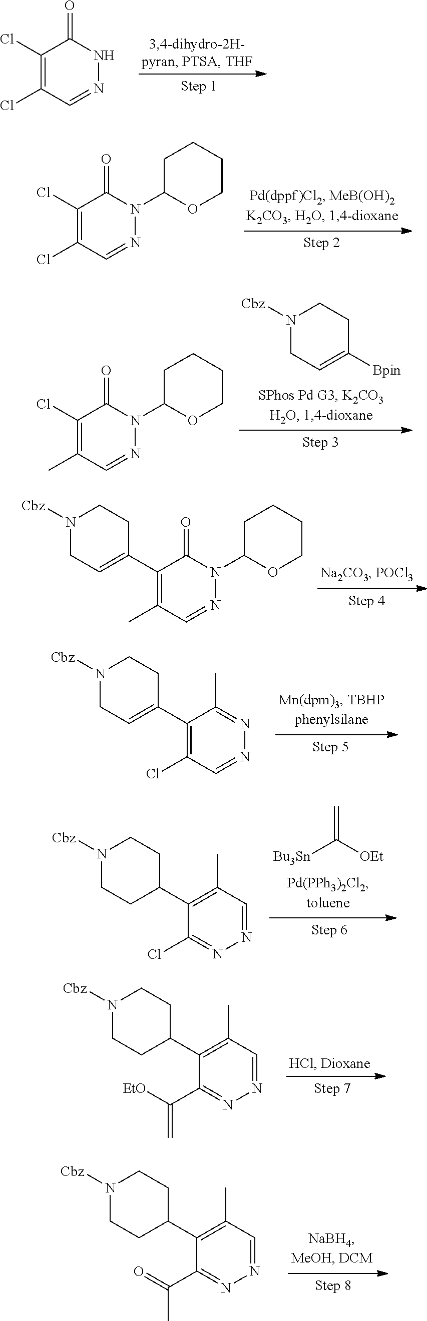

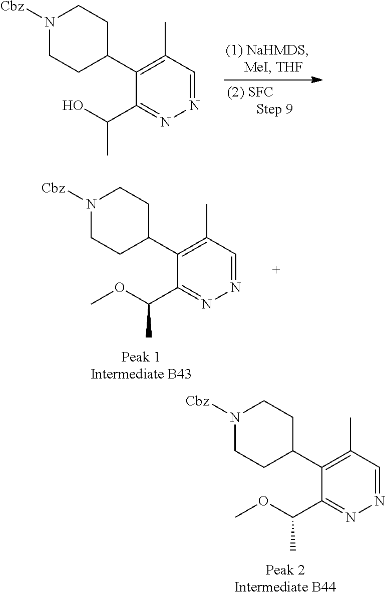

Step 1. 4,5-Dichloro-2-(tetrahydro-2H-pyran-2-yl)pyridazin-3 (2H)-one. A mixture of 4-methylbenzenesulfonic acid hydrate (1.11 g, 5.82 mmol, Sigma-Aldrich), 4,5-dichloropyridazin-3 (2H)-one (12 g. 72.7 mmol, Combi-Blocks Inc.), and 3,4-dihydro-2H-pyran (8.63 mL, 95 mmol, Oakwood Products, Inc.) in THF (100 mL) was stirred at reflux for 18 h. An additional aliquot of 3,4-dihydro-2H-pyran (5 mL) was added at 16 h and then the reaction mixture was removed from heat and allowed to stir at rt for 48 h. The mixture was concentrated, dissolved in EtOAc (150 mL), and washed with 2 N NaOH (100 mL). The organic layer was taken, dried over MgSO₄, filtered, and concentrated to afford the crude product which was chromatographed, eluting with a gradient of 0% to 50% EtOAc in heptane, to provide 4,5-dichloro-2-(tetrahydro-2H-pyran-2-yl)pyridazin-3 (2H)-one, m/z (ESI): 165.0 (M−THP+H)⁺. ¹H NMR (400 MHz, DMSO-d₆): δ (ppm) 8.24 (s, 1H), 5.85 (dd, J=10.4, 2.1 Hz, 1H), 3.93-4.01 (m, 1H), 3.62-3.67 (m, 1H), 1.99-2.10 (m, 1H), 1.91-1.97 (m, 1H), 1.63-1.74 (m, 2H), 1.46-1.57 (m, 2H).

Step 2. 4-Chloro-5-methyl-2-(tetrahydro-2H-pyran-2-yl)pyridazin-3 (2H)-one. A mixture of Pd(dppf)Cl₂ DCM (0.393 g, 0.482 mmol, Strem Chemicals, Inc.), methylboronic acid (0.529 g. 8.83 mmol, Oakwood Products, Inc.), 4,5-dichloro-2-(tetrahydro-2H-pyran-2-yl)pyridazin-3 (2H)-one (2 g, 8.03 mmol), and K₂CO₃ anhydrous (2.77 g, 20.07 mmol) was purged with N₂. Then, the mixture was dissolved in 1,4-dioxane/water (20/2 mL) and heated at 95° C. for 17 h. The reaction mixture was cooled to rt, diluted with water, and extracted with EtOAc. The combined organic extracts were dried over Na₂SO₄, filtered, concentrated, and purified by chromatography on silica gel using 0-70% EtOAc in heptane to afford 4-chloro-5-methyl-2-(tetrahydro-2H-pyran-2-yl)pyridazin-3 (2H)-one, m/z (ESI): 229.1 (M+H)⁺

Step 3. Benzyl 4-(5-methyl-3-oxo-2-(tetrahydro-2H-pyran-2-yl)-2,3-dihydropyridazin-4-yl)-3,6-dihydropyridine-1 (2H)-carboxylate. To a glass vial was added K₂CO₃ (907 mg, 6.56 mmol, Sigma-Aldrich Corporation), SPhos Pd G3 (227 mg, 0.262 mmol, Sigma-Aldrich Corporation), (1-((benzyloxy) carbonyl)-1,2,3,6-tetrahydropyridin-4-yl) boronic acid (1081 mg, 3.15 mmol, Combi-Blocks Inc.), and 4-chloro-5-methyl-2-(tetrahydro-2H-pyran-2-yl)pyridazin-3 (2H)-one (600 mg, 2.62 mmol), in 1,4-dioxane (6 mL) and water (0.6 mL). The reaction mixture was bubbled with N₂ for 15 min before heating at 95° C. for 2 h. The reaction was quenched by 5 mL satd. NH₄Cl and extracted with EtOAc (3×5 mL). The combined organic extracts were washed with brine, dried over Na₂SO₄, and concentrated. The crude material was purified by column chromatography, eluting with acetone in heptane (5-50%) to afford benzyl 4-(5-methyl-3-oxo-2-(tetrahydro-2H-pyran-2-yl)-2,3-dihydropyridazin-4-yl)-3,6-dihydropyridine-1 (2H)-carboxylate, m/z (ESI): 410.0 (M+H)⁺.

Step 4. Benzyl 4-(3-chloro-5-methylpyridazin-4-yl)-3,6-dihydropyridine-1 (2H)-carboxylate. A mixture of POCl₃ (910 μL, 9.77 mmol, Sigma-Aldrich Corporation) and benzyl 4-(5-methyl-3-oxo-2-(tetrahydro-2H-pyran-2-yl)-2,3-dihydropyridazin-4-yl)-3,6-dihydropyridine-1 (2H)-carboxylate (800 mg, 1.95 mmol) was heated to 80° C. for 1 h, then quenched by 10% Na₂CO₃ aq solution. The mixture was extracted with DCM (3×2 mL) and the combined organic extracts were dried over Na₂SO₄, filtered, and concentrated under vacuum. The crude material was purified by column chromatography, eluting with a gradient of 5-30% acetone in heptane to afford benzyl 4-(3-chloro-5-methylpyridazin-4-yl)-3,6-dihydropyridine-1 (2H)-carboxylate (410 mg, 1.193 mmol, 61% yield), m/z (ESI): 344.2 (M+H)⁺).

Step 5. Benzyl 4-(3-chloro-5-methylpyridazin-4-yl)piperidine-1-carboxylate. To a degassed solution of benzyl 4-(3-chloro-5-methylpyridazin-4-yl)-3,6-dihydropyridine-1 (2H)-carboxylate (3 g, 8.73 mmol) and Mn (dpm); (1.055 g, 1.745 mmol, Strem Chemicals, Inc.) in IPA (100 mL) at 0° C. was added TBHP (5 M in nonane, 5.24 mL, 26.2 mmol, Sigma-Aldrich Corporation) and phenylsilane (2.83 mL, 26.2 mmol, Chem Impex) dropwise at 0° C. The solution was warmed to rt and stirred for 2 h. The reaction was quenched by the addition of NH₄OH, water, and EtOAc. The organic phase was separated, and the aqueous phase was extracted with EtOAc. The organic extracts were washed with brine, dried over Na₂SO₄, filtered, concentrated, and purified by chromatography using 0-100% EtOAc in heptane to afford benzyl 4-(3-chloro-5-methylpyridazin-4-yl)piperidine-1-carboxylate (2.4 g. 6.94 mmol, 80% yield), m/z (ESI): 346.0 (M+H)⁺.

Step 6: Benzyl 4-(3-(1-ethoxyvinyl)-5-methylpyridazin-4-yl)piperidine-1-carboxylate. Pd(PPh₃)₂Cl₂ (0.325 g, 0.463 mmol, Sigma-Aldrich Corporation) and benzyl 4-(3-chloro-5-methylpyridazin-4-yl)piperidine-1-carboxylate (1.6 g, 4.63 mmol) were dissolved in toluene (20 mL) and tributyl(1-ethoxyvinyl) tin (3.13 mL, 9.25 mmol, Asta Tech, Inc) was added. The reaction mixture was degassed with N₂ for 10 min, then the temperature was increased to 110° C., and the reaction was stirred for 12 h. The reaction was purified by chromatography, eluting with a gradient of 0-100% EtOAc in heptane to afford benzyl 4-(3-(1-ethoxy vinyl)-5-methylpyridazin-4-y 1) piperidine-1-carboxy late (1.45 g, 3.79 mmol, 82% yield), m/z (ESI): 382.2 (M+H)⁺.

Step 7: Benzyl 4-(3-acetyl-5-methylpyridazin-4-yl)piperidine-1-carboxylate. To a solution of benzyl 4-(3-(1-ethoxy vinyl)-5-methylpyridazin-4-yl)piperidine-1-carboxylate (1.5 g, 3.93 mmol) in 1,4-dioxane (10 mL) and water (2 mL) at 0° C. was added HCl (4 M in dioxane) (1.43 mL, 39.3 mmol, Sigma-Aldrich Corporation). The reaction mixture was allowed to stir for 12 h and quenched by aqueous K₂CO₃ solution (2 M, 10 mL). The mixture was diluted with water and extracted with EtOAc. The combined organics extracts were dried over Na₂SO₄, filtered, and concentrated to give benzyl 4-(3-acetyl-5-methylpyridazin-4-yl)piperidine-1-carboxylate, which was used directly in the next step, m/z (ESI): 354.3 (M+H)⁺.

Step 8: Benzyl 4-(3-(1-hydroxyethyl)-5-methylpyridazin-4-yl)piperidine-1-carboxylate. Benzyl 4-(3-acetyl-5-methylpyridazin-4-yl)piperidine-1-carboxylate was dissolved in MeOH (15 mL) and DCM (5 mL), and NaBH₄ (0.223 g, 5.90 mmol, Sigma-Aldrich Corporation) was added portion-wise. After 15 min, the reaction was quenched with acetone, diluted with water, and extracted with EtOAc. The combined organics extracts were dried over Na₂SO₄, filtered, and concentrated. The crude mixture was purified by chromatography, eluting with a gradient of 0-100% EtOAc in heptane to give benzyl 4-(3-(1-hydroxyethyl)-5-methylpyridazin-4-yl)piperidine-1-carboxylate (1.4 g, 3.94 mmol, quant. yield), m/z (ESI): 356.2 (M+H)⁺.

Step 9: Benzyl 4-(3-(1-methoxyethyl)-5-methylpyridazin-4-yl)piperidine-1-carboxylate. To a solution of benzyl 4-(3-(1-hydroxyethyl)-5-methylpyridazin-4-yl)piperidine-1-carboxylate (1.4 g. 3.94 mmol) in THF (15 mL) at −78° C. was added NaHMDS (5.12 mL, 5.12 mmol, 1 M solution in THF) and the mixture was allowed to stir at −78° C. for 5 min. McI (0.566 mL, 9.06 mmol) was added and, the reaction was allowed to stir for 15 min for −78° C. Then, the reaction was warmed to rt and stirred for 30 min. The reaction was quenched with satd. NH₄Cl, diluted with water, and extracted with EtOAc. The combined organics extracts were dried over Na₂SO₄, filtered, concentrated. The crude mixture was purified by chromatography, eluting with a gradient of 0-100% EtOAc in heptane to afford benzyl 4-(3-(1-methoxyethyl)-5-methylpyridazin-4-yl)piperidine-1-carboxylate (960 mg, 2.60 mmol, 66% yield), m/z (ESI): 370.2 (M+H)⁺.

A racemic mixture of benzyl 4-(3-(1-methoxyethyl)-5-methylpyridazin-4-yl)piperidine-1-carboxy late was purified by SFC using a Chiralcel OX, 2×25 cm 5 μm column with a mobile phase of 25% 1:1 ACN: MeOH using a flowrate of 80 mL/min to obtain a 1^{st} eluting isomer and a 2^{nd} eluting isomer. The stereochemistry of the isomers was assigned arbitrarily to be benzyl(R)-4-(3-(1-methoxyethyl)-5-methylpyridazin-4-yl)piperidine-1-carboxylate, Intermediate B43 as the 1st eluting isomer and benzyl(S)-4-(3-(1-methoxyethyl)-5-methylpyridazin-4-yl)piperidine-1-carboxylate, Intermediate B44 as the 2″4 eluting isomer.

Intermediates in Table 1-9 were prepared following the procedure described for Intermediates B45 and B46, using appropriate starting materials and amination conditions rather than Stille conditions. All starting materials are commercially available or are described above.

TABLE 1-9

| | Chemical Structure & Name | LCMS: (ESI + ve ion) m/z; NMR | Comments |
|---|---|---|---|
| B45 | 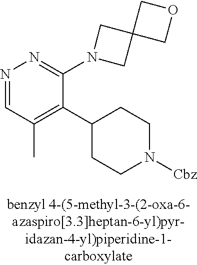 [SMILES: Cc1cnnc(N2CC3(COC3)C2)c1C1CCN(C(=O)OCc2ccccc2)CC1] | m/z (ESI): 409.3 (M + H)⁺. | Steps 7-9 were omitted; Step 6 was performed before Step 5 |
| B46 | 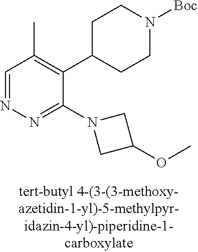 [SMILES: COC1CN(c2nncc(C)c2C2CCN(C(=O)OC(C)(C)C)CC2)C1] | m/z (ESI): 363.0 (M + H)⁺. | Steps 7-9 were omitted: Step 6 was performed before Step 5 |

### Intermediate C1—tert-Butyl(R)-3-methyl-4-(4-methyl-1-(oxetan-3-yl)-1H-pyrazol-5-yl)piperazine-1-carboxylate

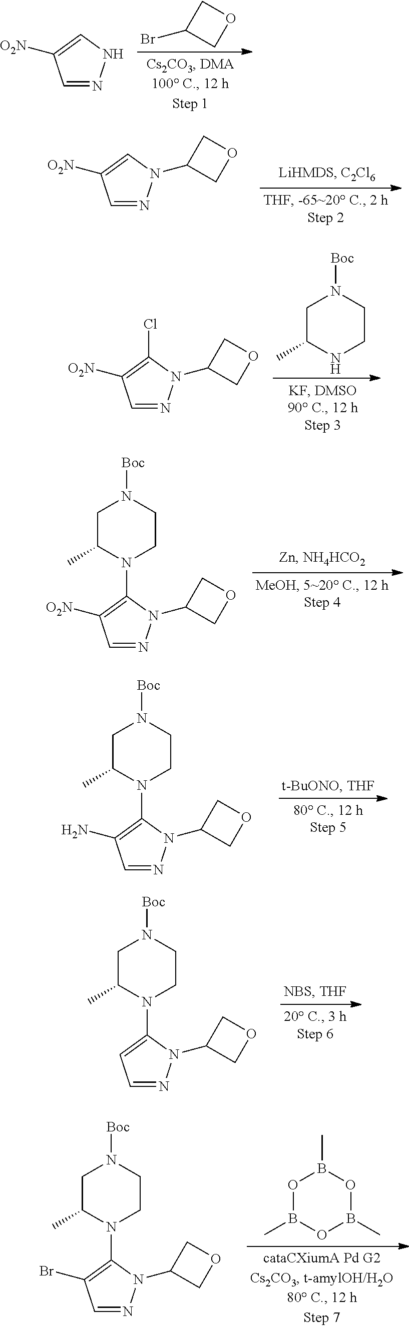

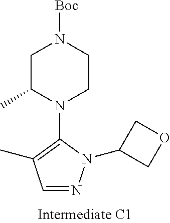 [SMILES: Cc1cnn(C2COC2)c1N1CCN(C(=O)OC(C)(C)C)C[C@H]1C]

Step 1. 4-Nitro-1-(oxetan-3-yl)-1H-pyrazole. To a solution of 4-nitro-1H-pyrazole (25 g, 221 mmol) in DMA (200 mL) was added 3-bromooxetane (36.3 g, 265 mmol) and cesium carbonate (72.0 g, 221 mmol). The mixture was stirred at 100° C. for 12 h. The reaction mixture was filtered, and the filtrate was diluted with H₂O (250 mL) and extracted with EtOAce (125 mL×3). The combined organic layers were washed with brine (250 mL×3), dried over Na₂SO₄, filtered, and concentrated under reduced pressure to give the crude 4-nitro-1-(oxetan-3-yl)-1H-pyrazole (37.5 g), which was used in the next step without further purification. ¹H NMR (400 MHz, CDCl₃), 8.32 (s, 1H), 8.18 (s, 1H), 5.40-5.55 (m, 1H), 4.95-5.15 (m, 4H).

Step 2. 5-Chloro-4-nitro-1-(oxetan-3-yl)-1H-pyrazole. To a solution of LiHMDS (1 M solution in THF, 473 mL, 473 mmol) in THF (400 mL) was added 4-nitro-1-(oxetan-3-yl)-1H-pyrazole (40 g, 236 mmol) in THF (300 mL) at −65° C. The mixture was stirred at −65° C. for 0.5 h. To the reaction mixture was added a solution of C₂Cl (67.2 g, 284 mmol) in THF (200 mL) at −65° C. The mixture was stirred at −65° C. for 0.5 h and then stirred at 20° C. for 1 h. The reaction mixture was quenched by the addition of satd. NH₄Cl (300 mL) and extracted with EtOAc (300 mL×3). The combined organic extracts were dried over Na₂SO₄, filtered, and concentrated under reduced pressure to give 5-chloro-4-nitro-1-(oxetan-3-yl)-1H-pyrazole (39.8 g, 196 mmol, 83% yield). ¹H NMR (400 MHz, CDCl₃), 8.61 (s, 1H), 5.70-5.85 (m, 1H), 4.85-5.00 (m, 4H).

Step 3. tert-Butyl(R)-3-methyl-4-(4-nitro-1-(oxetan-3-yl)-1H-pyrazol-5-yl)piperazine-1-carboxylate. A mixture of 5-chloro-4-nitro-1-(oxetan-3-yl)-1H-pyrazole (8 g, 39.3 mmol), tert-butyl(R)-3-methylpiperazine-1-carboxylate (7.87 g, 39.3 mmol) and potassium fluoride (13.70 g, 236 mmol) in DMSO (150 mL) was stirred at 90° C. for 12 h. The resulting mixture was diluted with water (300 mL) and extracted with EtOAc (200 mL×3). The combined organic layers were washed with brine (500 mL), dried over Na₂SO₄, filtered and concentrated under reduced pressure. The residue was purified by chromatography (eluting with Pet. ether/EtOAc=5:1 to 1:1) to obtain tert-butyl(R)-3-methyl-4-(4-nitro-1-(oxetan-3-yl)-1H-pyrazol-5-yl)piperazine-1-carboxylate (12 g, 32.7 mmol, 83% yield).

Step 4. tert-Butyl(R)-4-(4-amino-1-(oxetan-3-yl)-1H-pyrazol-5-yl)-3-methylpiperazine-1-carboxylate. To a solution of tert-butyl(R)-3-methyl-4-(4-nitro-1-(oxetan-3-yl)-1H-pyrazol-5-yl)piperazine-1-carboxylate (42.85 g, 117 mmol) and ammonium formate (29.4 g, 467 mmol) in MeOH (900 mL) was added zinc (31.57 g, 483 mmol) in portions at 5° C. The resulting mixture was stirred at 20° C. for 12 h. The reaction mixture was filtered and concentrated under reduced pressure. The crude product was purified by chromatography (eluting with Pet. ether/EtOAc=33-100%) to give tert-butyl(R)-4-(4-amino-1-(oxetan-3-yl)-1H-pyrazol-5-yl)-3-methylpipcrazine-1-carboxylate (25.5 g, 76 mmol, 65% yield), m/z (ESI): 282.3 [M-Bu+H]⁺.

Step 5. tert-Butyl(R)-3-methyl-4-(1-(oxetan-3-yl)-1H-pyrazol-5-yl)piperazine-1-carboxylate. A mixture of tert-butyl(R)-4-(4-amino-1-(oxetan-3-yl)-1H-pyrazol-5-yl)-3-methylpiperazine-1-carboxylate (25.5 g, 76 mmol) and tert-butyl nitrite (11.69 g, 113 mmol) in THF (500 mL) was degassed and purged with N₂ 3 times, and then the mixture was stirred at 80° C. for 12 h under N₂ atmosphere. The reaction mixture was concentrated and purified by chromatography (eluting with Pet. ether/EtOAc=33-100%) to obtain tert-butyl(R)-3-methyl-4-(1-(oxetan-3-yl)-1H-pyrazol-5-yl)piperazine-1-carboxylate (18.7 g, 58 mmol, 77% yield), m/z (ESI): 323.3 [M+H]⁺.

Step 6. tert-Butyl(R)-4-(4-bromo-1-(oxetan-3-yl)-1H-pyrazol-5-yl)-3-methylpiperazine-1-carboxylate. To a solution of tert-butyl(R)-3-methyl-4-(1-(oxetan-3-yl)-1H-pyrazol-5-yl)piperazine-1-carboxylate (37.4 g, 116 mmol) in THF (700 mL) was added NBS (18.58 g, 104 mmol). The reaction mixture was stirred at 20° C. for 3 hr. The reaction mixture was concentrated and purified by chromatography (eluting with pet. ether/EtOAc=5-25%). tert-butyl(S)-4-(4-bromo-1-(oxetan-3-yl)-1H-pyrazol-5-yl)-3-methylpiperazine-1-carboxylate (39.5 g, 98.5 mmol, 85% yield) was obtained, m/z (ESI): 401.1 and 403.2 (M+H)⁺. 3H NMR (400 MHz, CDCl₃), 7.51 (s, 1H), 5.70-5.80 (m, 1H), 5.05-5.15 (m, 2H), 4.85-4.95 (m, 2H), 3.95-4.05 (m, 2H), 3.35-3.45 (m, 2H), 2.50-3.05 (m, 3H), 1.49 (s, 9H), 0.74 (d, J=4.0 Hz, 3H). SFC: 99.35% ee.

Step 7. tert-Butyl(R)-3-methyl-4-(4-methyl-1-(oxetan-3-yl)-1H-pyrazol-5-yl)piperazine-1-carboxylate. A mixture of tert-butyl(R)-4-(4-bromo-1-(oxetan-3-yl)-1H-pyrazol-5-yl)-3-methylpiperazine-1-carboxylate (39.5 g, 98 mmol), Cs₂CO₃(64.1 g, 197 mmol), cataCXiumA Pd G₂ (6.58 g, 9.84 mmol), and 2,4,6-trimethyl-1,3,5,2,4,6-trioxatriborinane (56.2 mL, 197 mmol) was added t-amylOH (500 mL) and water (50 mL) and was degassed. The mixture was stirred at 80° C. for 12 h under N₂ atmosphere. The mixture was purified by chromatography (eluting with Pet. ether/EtOAc=3-12.5%). Purification by prep-HPLC: Column: Welch Xtimate C18 250×100 mm #10 μm; water (NH₃H₂O+NH₄HCO₃)-ACN; flow rate 280 mL/min gave tert-butyl(R)-3-methyl-4-(4-methyl-1-(oxetan-3-yl)-1H-pyrazol-5-yl)piperazine-1-carboxylate (17.9 g, 53 mmol, 54% yield), m/z (ESI): 337.1 (M+H)⁺. ¹H NMR (400 MHz, CDCl₃), 7.32 (s, 1H), 5.60-5.75 (m, 1H), 5.05-5.15 (m, 2H), 4.85-4.95 (m, 2H), 4.00 (br s, 2H), 3.05-3.20 (m, 2H), 2.70-3.00 (m, 2H), 2.58 (br s, 1H), 2.03 (s. 3H), 1.47 (s, 9H), 0.67 (d, J=5.5 Hz, 3H).

### Intermediate D1 (TFA Salt)—Benzyl 4-((2S,3R)-2-allylazetidin-3-yl)piperazine-1-carboxylate

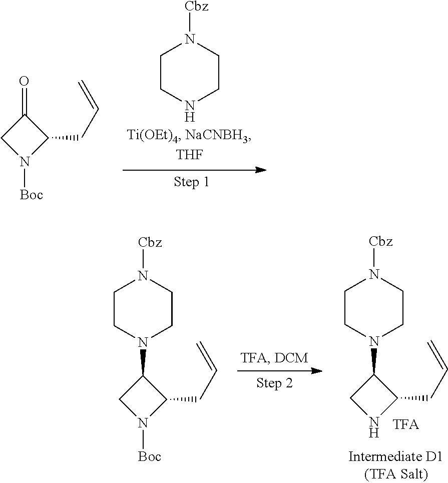 [SMILES: C=CC[C@H]1C(=O)CN1C(=O)OC(C)(C)C ; O=C(OCc1ccccc1)N1CCNCC1 ; C=CC[C@H]1[C@H](N2CCN(C(=O)OCc3ccccc3)CC2)CN1C(=O)OC(C)(C)C ; C=CC[C@@H]1NC[C@H]1N1CCN(C(=O)OCc2ccccc2)CC1]

Step 1. Benzyl 4-((2S,3R)-2-allyl-1-(tert-butoxycarbonyl)azetidin-3-yl)piperazine-1-carboxylate. To a stirred solution of tert-butyl(S)-2-allyl-3-oxoazetidine-1-carboxylate (225 g, 1065 mmol) in THF (6.75 mL), were added sodium cyanoborohydride (352 g, 1598 mmol) and titanium (IV) ethoxide (447 mL., 2130 mmol) dropwise at 0° C., and the reaction was stirred at rt for 24 h. The reaction mixture was cooled to −78° C., and sodium cyanoborohydride (134 g, 2130 mmol) was added and stirred for 1 h at −78° C. The reaction mixture was quenched with water (1 L) and extracted with EtOAc (4×1 L). The combined organic extracts were filtered through a celite bed and washed with EtOAc (10 L). The organic layer was washed with brine (2 L), dried (Na₂SO₄), filtered, and concentrated under reduced pressure. The crude material was purified by flash chromatography eluting with 25%-35% EtOAc in hexanes to afford 228 g of trans mixture and 77 g of cis mixture.

The mixture of trans isomers was purified by SFC using a LUX-C4 (250×50) mm, 5 μm, column with a mobile phase of liquid CO₂:[ACN:IPA(1:1)] (85:15) using a flowrate of 180 mL/min to obtain a 1^{st} eluting isomer and a 2^{nd} eluting isomer. The stereochemistry of the isomers was assigned to be benzyl 4-((28,3R)-2-allyl-1-(tert-butoxycarbonyl)azetidin-3-yl)piperazine-1-carboxylate as the 1^{st} eluting isomer and benzyl 4-((2R,3S)-2-allyl-1-(tert-butoxycarbonyl)azetidin-3-yl)piperazine-1-carboxylate as the 2°d eluting isomer. 15 Eluting isomer: m/z (ESI): 416.3 (M+H)⁺. ¹H NMR (400 MHz, DMSO-d₆): δ (ppm) 7.57-7.06 (m, 5H), 5.93-5.66 (m, 1H), 5.28-4.83 (m, 4H), 3.96 (dt, J=7.1, 4.5 Hz, 1H), 3.72 (s, 1H), 3.53-3.62 (m, 1H), 3.52 (dd, J=8.7, 4.9 Hz, 4H), 2.69 (dd, J=7.9, 3.5 Hz, 1H), 2.47-2.33 (m, 2H), 2.25 (dd, J=11.2, 6.2 Hz, 4H), 1.38 (s, 9H).

Step 2. Benzyl 4-((2S,3R)-2-allylazetidin-3-yl)piperazine-1-carboxylate, Intermediate D1, To a stirred solution of benzyl 4-((28,3R)-2-allyl-1-(tert-butoxycarbonyl)azetidin-3-yl)piperazine-1-carboxylate (370 g, 890 mmol) in DCM (1.85 L), was added TFA (1.11 L, 14240 mmol) dropwise at 0° C., and stirred at rt for 2 h. The reaction mixture was concentrated under reduced pressure, co-evaporated with toluene (3×500 mL), triturated with diethyl ether (500 mL), and dried under vacuum to afford Intermediate D1 (370 g, crude as TFA salt). The crude product was taken for next step without further purification, m/z (ESI): 316.3 (M+H)⁺.

Intermediates in Table 1-10 were prepared following the procedure described for Intermediate D1, using appropriate starting materials. All starting materials are commercially available or are described above.

TABLE 1-10

| | Chemical Structure & Name | LCMS: (ESI + ve ion) m/z; NMR | Comments |
|---|---|---|---|
| D2 (TFA Salt) | 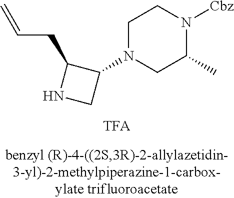 [SMILES: C=CC[C@@H]1NC[C@H]1N1CCN(C(=O)OCc2ccccc2)[C@H](C)C1] | ¹H NMR (400 MHz, DMSO-d₆) δ 8.93 (d, J = 77.0 Hz, 2H), 7.43-7.28 (m, 5H), 5.74 (ddt, J = 17.1, 10.3, 6.7 Hz, 1H), 5.29-5.03 (m, 4H), 4.44-4.18 (m, 2H), 3.97-3.73 (m, 3H), 3.25 (d, J = 7.4 Hz, 1H), 3.09 (t, J = 12.3 Hz, 1H), 2.93 (d, J = 11.1 Hz, 1H), 2.75-2.53 (m, 3H), 2.24 (d, J = 10.9 Hz, 1H), 2.08 (d, J = 12.7 Hz, 1H), 1.29-1.17 (m, 3H). | |

### Intermediate D3-Benzyl 4-((2S,3R)-2-(3-hydroxypropyl)azetidin-3-yl)piperazine-1-carboxylate

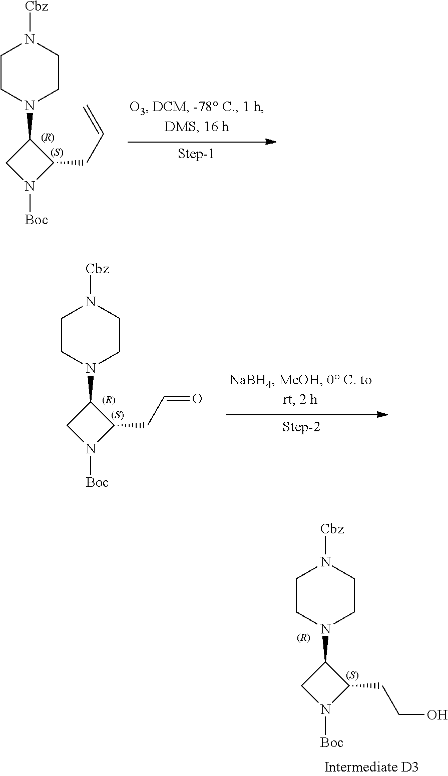

Step 1. Benzyl 4-((28,3R)-1-(tert-butoxycarbonyl)-2-(2-oxoethyl)azetidin-3-yl)piperazine-1-carboxylate. To a stirred solution of benzyl 4-((2S,3R)-2-allyl-1-(tert-butoxycarbonyl)azetidin-3-yl)piperazine-1-carboxylate (6 g, 14.44 mmol) in DCM (60 mL) was purged with ozone gas (Chemlabs Ozonizer) at −78° C. for 1 min. The reaction mixture was quenched with BH₃-DMS (0.5 mL) at 0° C. followed by ice cold water (50 mL) and extracted with DCM (3×150 mL). The combined organic extracts were dried (Na₂SO₄), filtered, and concentrated under reduced pressure to give benzyl 4-((2S,3R)-1-(tert-butoxycarbonyl)-2-(2-oxoethyl)azetidin-3-yl)piperazine-1-carboxylate (6 g, crude). The crude compound was used for next step without any purification.

Step 2. Benzyl 4-((2S,3R)-1-(tert-butoxycarbonyl)-2-(2-hydroxyethyl)azetidin-3-yl)piperazine-1-carboxylate (Intermediate D3). To a stirred solution of benzyl 4-((2S,3R)-1-(tert-butoxycarbonyl)-2-(2-oxoethyl)azetidin-3-yl)piperazine-1-carboxylate (6 g, 14.37 mmol) in MeOH (120 mL) was added NaBH₄ (1.35 g, 35.9 mmol) in portions at 0° C. under N₂ atmosphere. The reaction mixture was stirred at rt for 1 h. The reaction mixture was quenched with ice cold water (50 mL) and extracted with EtOAc (3×100 mL). The combined organic extracts were dried (Na₂SO₄), filtered, and concentrated under reduced pressure. The crude compound was purified by SFC (using a Chiralpak IG, 250×50 mm, 5 μm column with a mobile phase of liquid CO₂: MeOH (55:45) using a flowrate of 180 mL/min) to afford benzyl 4-((2S,3R)-1-(tert-butoxycarbonyl)-2-(2-hydroxyethyl)azetidin-3-yl)piperazine-1-carboxylate (2.15 g, 36% yield), m/z (ESI): 420.1 (M+H)⁺. 1H NMR (400 MHz, DMSO-d₆): δ 7.43-7.28 (m, 5H), 5.08 (s, 2H), 4.45 (t, J=4.9 Hz, 1H), 3.99 (q. J=6.5, 5.9 Hz, 1H), 3.76 (br s, 1H), 3.50-3.29 (m, 7H), 2.70-2.63 (m, 1H), 2.20-2.40 (m, 4H), 1.92 (dq, J=13.3, 6.9 Hz, 1H), 1.70 (dq, J=13.2, 6.4 Hz, 1H), 1.38 (s, 9H).

Intermediates in Table 1-11 were prepared following the procedure described for Intermediate D3, using appropriate starting materials. All starting materials are commercially available or are described above.

TABLE 1-11

| | Chemical Structure & | | |
|---|---|---|---|
| | Name | LCMS: (ESI + ve ion) m/z; NMR | Comments |
| D4 | 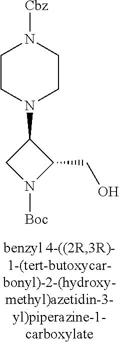 [SMILES: CC(C)(C)OC(=O)N1C[C@@H](N2CCN(C(=O)OCc3ccccc3)CC2)[C@@H]1CO] | m/z (ESI): 406.0 (M + H)⁺. | |

### Intermediate E1-Benzyl 4-((7aS,8R)-2-chloro-4-(difluoromethyl)-7,7a,8,9-tetrahydroazeto[1,2-a]pyrido[3,4-f]azepin-8-yl)piperazine-1-carboxylate

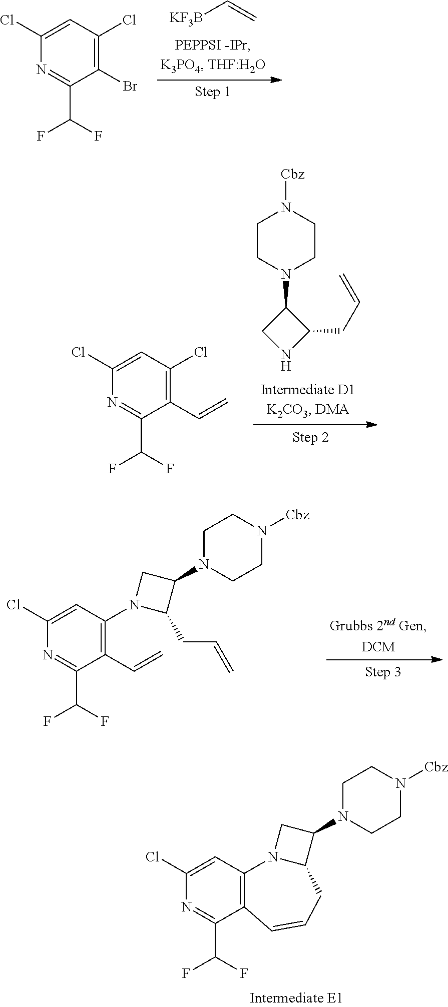

Step 1. 4,6-Dichloro-2-(difluoromethyl)-3-vinylpyridine. To a stirred solution of 3-bromo-4,6-dichloro-2-(difluoromethyl)pyridine (150 g, 542 mmol) in THF (2.4 L) and water (600 mL) were added potassium trifluoro (vinyl) borate (102 g, 758 mmol) and K₃PO₄ (345 g. 1625 mmol) and the solution was degassed and purged with N₂ for 5 min. PEPPSI-IPR catalyst (36.8 g. 54.2 mmol) was added and again degassed and purged with Na for 5 min and stirred at 85° C. for 16 h. The reaction mixture was cooled to rt, filtered, diluted with water (500 mL), and extracted with MTBE (3×500 mL). The combined organic extracts were washed with brine (500 mL), dried (Na₂SO₄), filtered, and concentrated. The crude material was purified by chromatography, eluting with a gradient of 0-100% EtOAc in hexanes to give 4,6-dichloro-2-(difluoromethyl)-3-vinylpyridine (74 g, 330 mmol, 61% yield). 3H NMR (400 MHz, DMSO-d₆): δ (ppm) 8.09 (s, 1H), 7.04 (t, J=53.1 Hz, 1H), 6.78 (ddt, J=17.8, 11.6, 1.8 Hz, 1H), 5.87 (dd, J=11.6, 1.1 Hz, 1H), 5.59 (dd, J=17.8, 1.0 Hz, 1H).

Step 2. Benzyl 4-((2S,3R)-2-allyl-1-(6-chloro-2-(difluoromethyl)-3-vinylpyridin-4-yl)azetidin-3-yl)piperazine-1-carboxylate. To a stirred solution of 4,6-dichloro-2-(difluoromethyl)-3-vinylpyridine (370.0 g, 895 mmol) in DMA (1.5 L) were added K₂CO₃ (866 g. 6264 mmol) and Intermediate D1 (200 g, 895 mmol) at 0° C., and stirred at 80° C. for 16 h. The reaction mixture was cooled to rt, quenched with ice cold water (6.0 L) and extracted with EtOAc (2×4 L). The combined organic extracts were washed with water (2×4 L) followed by brine (3×2 L), dried (Na₂SO₄), filtered, and concentrated under reduced pressure. The crude material was purified by flash chromatography, eluting with a gradient of 0 to 25% EtOAc:hexanes to give benzyl 4-((2S,3R)-2-allyl-1-(6-chloro-2-(difluoromethyl)-3-vinylpyridin-4-yl)azetidin-3-yl)piperazine-1-carboxylate (304 g. 604 mmol, 68% yield), m/z (ESI): 503.3 (M+H)⁺. ¹H NMR (400 MHz, DMSO-d₆): δ (ppm) 7.42-7.29 (m, 5H), 6.99-6.64 (m, 3H), 5.83 (ddt, J=17.2, 10.0, 7.2 Hz, 1H), 5.68 (dd, J=11.4, 1.6 Hz, 1H), 5.32 (dd, J=17.6, 1.7 Hz, 1H), 5.21-5.04 (m, 4H), 4.33-4.19 (m, 2H), 3.63 (dd, J=9.1, 4.4 Hz, 1H), 3.48-3.39 (m, 4H), 2.86 (dt, J=7.3, 4.1 Hz, 1H), 2.49-2.36 (m, 2H), 2.31 (d, J=5.4 Hz, 2H), 2.24 (d, J=11.5 Hz, 2H).

Step 3. Benzyl 4-((7aS,8R)-2-chloro-4-(difluoromethyl)-7,7a,8,9-tetrahydroazeto[1,2-a]pyrido[3,4-f]azepin-8-yl)piperazine-1-carboxylate, Intermediate E1. To a stirred solution of benzyl 4-((28,3R)-2-allyl-1-(6-chloro-2-(difluoromethyl)-3-vinylpyridin-4-yl)azetidin-3-yl)piperazine-1-carboxylate (120.5 g, 240 mmol) in DCM (3.6 L), was added Grubbs catalyst 2^{nd} generation (30.5 g, 35.9 mmol) at rt and stirred for 48 h. The reaction mixture was quenched with water (1.5 L) and extracted with DCM (2×1 L). The combined organic extracts were washed with water (2×2 L) followed by brine (2×1 L), dried (Na₂SO₄), filtered, and concentrated under reduced pressure. The crude material was purified by flash chromatography, eluting with a gradient of 0 to 25% EtOAc in hexanes to give Intermediate E1 (86 g, 181 mmol, 76% yield), m/z (ESI): 475.1 (M+H)⁺. ¹H NMR (400 MHz, DMSO-d₆): δ (ppm) 7.42-7.29 (m, 5H), 6.99 (t, J=53.8 Hz, 1H), 6.76-6.68 (m, 1H), 6.55 (s, 1H), 6.09 (ddd, J=12.5, 7.4, 3.3 Hz, 1H), 5.08 (s, 2H), 4.01 (t, J=7.9 Hz, 2H), 3.84 (t, J=7.6 Hz, 1H), 3.48-3.38 (m, 4H), 3.08 (q, J=6.6 Hz, 1H), 2.75 (ddd, J=17.0, 7.5, 2.7 Hz, 1H), 2.65 (dd, J=16.2, 13.0 Hz, 1H), 2.44-2.26 (m, 4H).

Intermediates in Table 1-12 were prepared following the procedure described for Intermediate E1, using appropriate starting materials. All starting materials are commercially available or are described above.

TABLE 1-12

| | Chemical Structure & Name | LCMS: (ESI + ve ion) m/z; NMR | Comments |
|---|---|---|---|
| E2 | 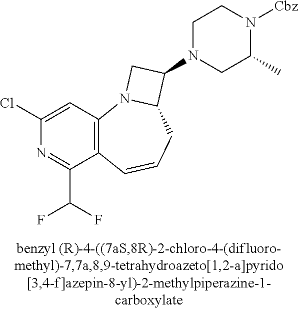 [SMILES: C[C@@H]1CN([C@@H]2CN3c4cc(Cl)nc(C(F)F)c4C=CC[C@@H]23)CCN1C(=O)OCc1ccccc1] | m/z (ESI): 489.2 (M + H)⁺. ¹H NMR (400 MHz, DMSO-d₆): δ 7.29-7.42 (m, 5H), 6.99 (t, J = 53.8 Hz, 1H), 6.71 (d, J = 12.5 Hz, 1H), 6.56 (s, 1H), 6.09 (ddd, J = 11.8, 7.3, 3.3 Hz, 1H), 5.02-5.17 (m, 2H), 4.22 (br s, 1H), 3.97-4.13 (m, 2H), 3.78 (dd, J = 10.5, 4.8 Hz, 2H), 2.95-3.13 (m, 2H), 2.83 (d, J = 11.1 Hz, 1H), 2.72 (dd, J = 7.4, 2.7 Hz, 1H), 2.66 (s, 1H), 2.57 (d, J = 11.1 Hz, 1H), 2.08 (dd, J = 11.2, 3.9 Hz, 1H), 1.89 (td, J = 11.6, 3.5 Hz, 1H), 1.19 (d, J = 6.8 Hz, 3H) | Step 2. Intermediate D2 was used |
| E3 | 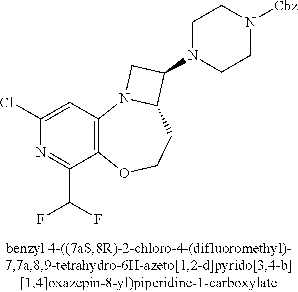 [SMILES: O=C(OCc1ccccc1)N1CCN([C@@H]2CN3c4cc(Cl)nc(C(F)F)c4OCC[C@@H]23)CC1] | m/z (ESI): 479.0 (M + H)⁺. | Step 1 was omitted; Step 2. Intermediate D3 and DIPEA in DMA was used; Step 3. Cs₂CO₃, Pd(OAc)₂, and TrixiePhos in toluene were used |

### Intermediate E4-Benzyl 4-((7aR,8R)-2-chloro-4-(difluoromethyl)-7-oxo-7,7a,8,9-tetrahydroazeto[1,2-a]pyrido[3,4-f]azepin-8-yl)piperazine-1-carboxylate

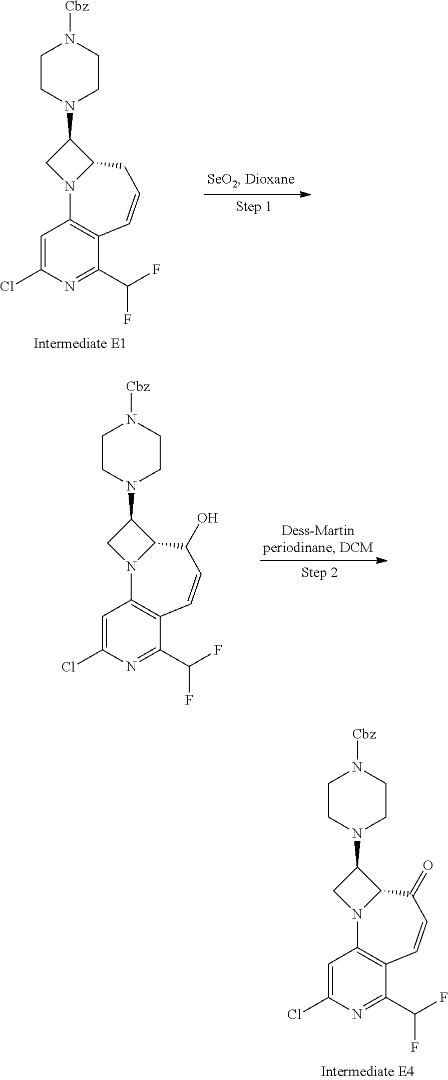

Step 1. Benzyl 4-((7S,7aR,8R)-2-chloro-4-(difluoromethyl)-7-hydroxy-7,7a,8,9-tetrahydroazeto[1,2-a]pyrido[3,4-f]azepin-8-yl)piperazine-1-carboxylate. Intermediate E1 (200 mg, 0.421 mmol) and selenium dioxide (140 mg, 1.263 mmol, Strem Chemicals, Inc.) in 1,4-dioxane (1.6 mL) were stirred at 105° C. for 6 h. The reaction mixture was filtered, concentrated, and purified by column chromatography, eluting with a gradient of 0%-40% acetone in heptane to give benzyl 4-((7S,7aR,8R)-2-chloro-4-(difluoromethyl)-7-hydroxy-7,7a,8,9-tetrahydroazeto[1,2-a]pyrido[3,4-f]azepin-8-yl)piperazine-1-carboxylate (82 mg, 0.17 mmol, 40% yield), m/z (ESI): 490.8 (M+H)⁺.

Step 2. Benzyl 4-((7aR,8R)-2-chloro-4-(difluoromethyl)-7-oxo-7,7a,8,9-tetrahydroazeto[1,2-a]pyrido[3,4-f]azepin-8-yl)piperazine-1-carboxylate, Intermediate E4. Benzyl 4-((7S,7aR,8R)-2-chloro-4-(difluoromethyl)-7-hydroxy-7,7a,8,9-tetrahydroazeto[1,2-a]pyrido[3,4-f]azepin-8-yl)piperazine-1-carboxylate (82 mg, 0.167 mmol) and Dess-Martin periodinane (106 mg, 0.251 mmol, Combi-Blocks Inc.) in DCM (3 mL) were stirred at rt for 5 h. The reaction was quenched by sat. aq. NaHCO₃ solution (2 mL) and extracted with DCM (2 mL×3). The combined organic extracts were washed with brine, dried over Na₂SO₄, and concentrated. The crude material was purified by column chromatography, eluting with a gradient of 5%-40% acetone in heptane to give Intermediate E4 (60 mg, 0.123 mmol, 74% yield), m/z (ESI): 489.0 (M+H)⁺.

Intermediates in Table 1-13 were prepared following the procedure described for Intermediate E4, using appropriate starting materials. All starting materials are commercially available or are described above.

TABLE 1-13

| | Chemical Structure & Name | LCMS: (ESI + ve ion) m/z; NMR | Comments |
|---|---|---|---|
| E5 | 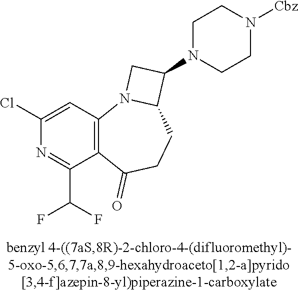 [SMILES: O=C1CC[C@H]2[C@H](N3CCN(C(=O)OCc4ccccc4)CC3)CN2c2cc(Cl)nc(C(F)F)c21] | m/z (ESI): 490.9 (M + H)⁺. | Step 1. dimethyl sulfide borane and hydrogen peroxide were used |
| E6 | 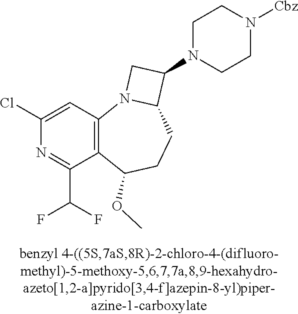 [SMILES: CO[C@H]1CC[C@H]2[C@H](N3CCN(C(=O)OCc4ccccc4)CC3)CN2c2cc(Cl)nc(C(F)F)c21] | m/z (ESI): 507.0 (M + H)⁺. | Step 1. dimethyl sulfide borane and hydrogen peroxide were used Step 2. NaH and Mel in THF were used |

### Intermediate E7-Benzyl 4-((7aR,8R)-2-chloro-4-(difluoromethyl)-7,7-difluoro-7,7a,8,9-tetrahydroazeto[1,2-a]pyrido[3,4-f]azepin-8-yl)piperazine-1-carboxylate

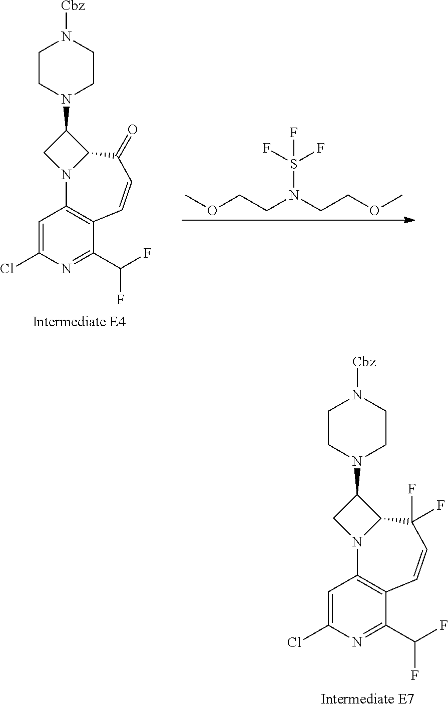

A solution of Intermediate E4 (38 mg, 0.078 mmol), and bis(2-methoxyethyl)aminosulfur trifluoride (1 mL, 5.42 mmol, AstaTech, Inc) was mixed at rt and stirred at 60° C. for 18 h. The reaction mixture was diluted with DCM (15 mL), quenched by sat. aq. NaHCO₃ solution (10 mL.), and extracted with DCM (5 mL×3). The combined organic extracts were washed with brine, dried over Na₂SO₃, and concentrated. The crude material was purified by column chromatography, eluting with a gradient of 0% to 40% acetone in heptane to give Intermediate E7 (18 mg, 0.035 mmol, 45% yield), m/z (ESI): 510.8 (M+H)⁺.

### Intermediate E8-tert-Butyl 4-((7aS,8R)-2-chloro-4-(difluoromethyl)-5,6,7,7a,8,9-hexahydroazeto[1,2-a]pyrido[3,4-f]azepin-8-yl)piperazine-1-carboxylate

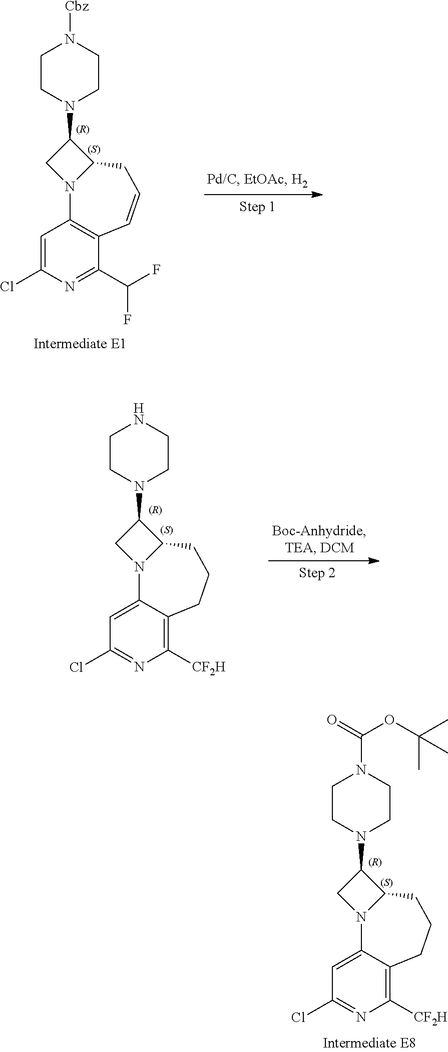

Step 1. (7aS,8R)-2-Chloro-4-(difluoromethyl)-8-(piperazin-1-yl)-5,6,7,7a,8,9-hexahydroazeto[1,2-a]pyrido[3,4-f]azepine. An autoclave was charged with benzyl 4-((7aS,8R)-2-chloro-4-(difluoromethyl)-7,7a,8,9-tetrahydroazeto[1,2-a]pyrido[3,4-f]azepin-8-yl)piperazine-1-carboxylate (4 g. 8.42 mmol) and EtOAc (200 mL). The reaction mixture was degassed and purged with N₂, Pd—C(10%, 0.896 g) was added to reaction mixture and stirred under H₂ gas atmosphere (52 psi) at rt for 4 h. The reaction mixture was filtered through a celite bed and washed with MeOH (200 mL). The filtrate was concentrated under reduced pressure to give (7aS,8R)-2-chloro-4-(difluoromethyl)-8-(piperazin-1-yl)-5,6,7,7a,8,9-hexahydroazeto[1,2-a]pyrido[3,4-f]azepine (3.6 g) that was used in the next step without further purification, m/z (ESI): 343.2 (M+H)⁺.

Step 2. tert-Butyl 4-((7aS,8R)-2-chloro-4-(difluoromethyl)-5,6,7,7a,8,9-hexahydroazeto[1,2-a]pyrido[3,4-f]azepin-8-yl)piperazine-1-carboxylate (Intermediate E8). To a stirred solution of (7aS,8R)-2-chloro-4-(difluoromethyl)-8-(piperazin-1-yl)-5,6,7,7a,8,9-hexahydroazeto[1,2-a]pyrido[3,4-f]azepine (3.6 g, 6.83 mmol) in DCM (72 mL) were added Et₃N (4.76 mL, 34.1 mmol) and Boc anhydride (3.96 mL, 17.06 mmol) at 0° C. under nitrogen atmosphere. The reaction mixture was stirred at rt for 16 h. The reaction mixture was quenched with water (50 mL) and extracted with DCM (2×100 mL). The combined organic extracts were dried (Na₂SO₄), filtered, and concentrated under reduced pressure. The crude material was purified by chromatography (silica gel, mobile phase 0-12% EtOAc in hexanes) to give tert-butyl 4-((7aS,8R)-2-chloro-4-(difluoromethyl)-5,6,7,7a,8,9-hexahydroazeto[1,2-a]pyrido[3,4-f]azepin-8-yl)piperazine-1-carboxylate (2.6 g, 70% yield over 2 steps), m/z (ESI): 443.2 (M+H)⁺. ¹H NMR (400 MHz, DMSO-d₆): δ ppm 6.90 (t, J=54.0 Hz, 1H), 6.50 (s, 1H), 4.27 (d, J=6.1 Hz, 1H), 3.86 (t, J=8.2 Hz, 1H), 3.78 (dd, J=8.9, 6.0 Hz, 1H), 3.34 (d, J=4.7 Hz, 4H), 2.99 (t, J=12.5 Hz, 1H), 2.88 (q,/=6.1 Hz, 1H), 2.62-2.55 (m, 1H), 2.38-2.25 (m, 4H), 1.97-1.75 (m, 3H), 1.75-1.65 (m, 1H), 1.40 (s, 9H).

### Intermediate E9-tert-Butyl 4-((7aS,8R)-2-chloro-4-(difluoromethyl)-6-hydroxy-5,6,7,7a,8,9-hexahydroazeto[1,2-a]pyrido[3,4-f]azepin-8-yl)piperazine-1-carboxylate

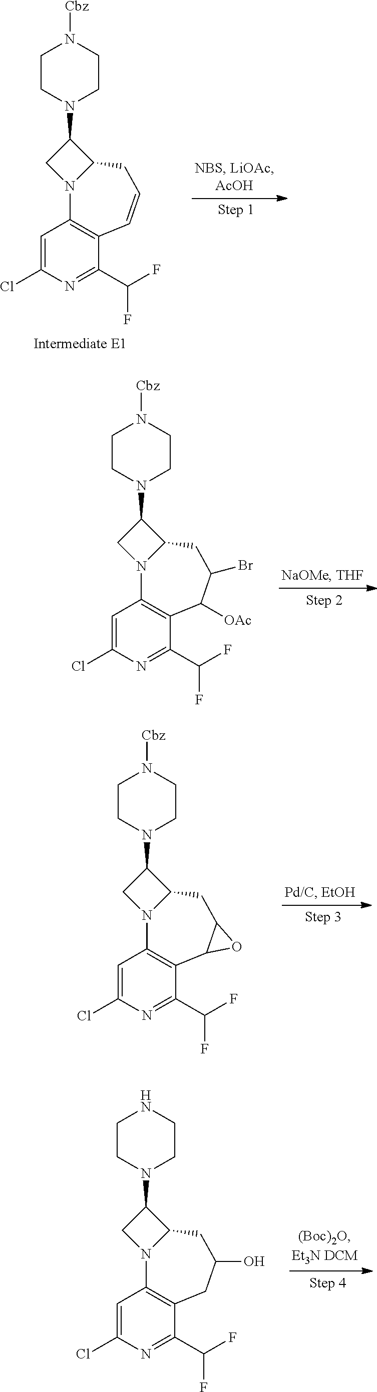

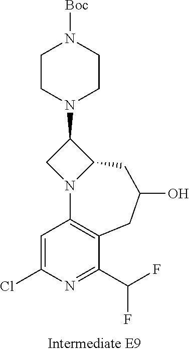

Step 1. Benzyl 4-((7aS,8R)-5-acetoxy-6-bromo-2-chloro-4-(difluoromethyl)-5,6,7,7a,8,9-hexahydroazeto[1,2-a]pyrido[3,4-f]azepin-8-yl)piperazine-1-carboxylate. A solution of Intermediate E1 (400 mg, 0.842 mmol), lithium acetate (222 mg, 3.37 mmol, TCI America) and NBS (157 mg, 0.884 mmol, Sigma-Aldrich Corporation) in AcOH (3 mL) was stirred at rt for 3 h. The reaction was concentrated and diluted using EtOAc (2 mL) and 10 wt % NaCO₃ aq solution (1 mL). The crude material was extracted with EtOAc (6 mL×3), washed with brine, dried over Na₂SO₄, and concentrated. The crude material was purified by column chromatography, eluting with a gradient of 3-40% acetone in heptane to give benzyl 4-((6aS,7R)-2-chloro-4-(difluoromethyl)-4b,5a,6,6a, 7,8-hexahydroazeto[1,2-a]oxireno[2,3-d]pyrido[3,4-f]azepin-7-yl)piperazine-1-carboxylate (505 mg, 0.823 mmol, 98% yield), m/z (ESI): 612.8 and 614.8 (M+H)⁺.

Step 2. Benzyl 4-((6aS,7R)-2-chloro-4-(difluoromethyl)-4b,5a,6,6a,7,8-hexahydroazeto[1,2-a]oxireno[2,3-d]pyrido[3,4-f]azepin-7-yl)piperazine-1-carboxylate. To a solution of benzyl 4-((7aS,8R)-5-acetoxy-6-bromo-2-chloro-4-(difluoromethyl)-5,6,7,7a,8,9-hexahydroazeto[1,2-a]pyrido[3,4-f]azepin-8-yl)piperazine-1-carboxylate (505 mg, 0.823 mmol) in THF (4.5 mL) was added sodium methoxide (222 mg, 4.11 mmol, Sigma-Aldrich Corporation). The reaction mixture was stirred at rt for 3 b. The reaction was quenched by water (5 mL) and extracted with EtOAc (3×3 mL). The combined organic extracts were washed with brine, dried through Na₂SO₄, and concentrated to give benzyl 4-((7aS,8R)-5-acetoxy-6-bromo-2-chloro-4-(difluoromethyl)-5,6,7,7a,8,9-hexahydroazeto[1,2-a]pyrido[3,4-f]azepin-8-yl)piperazine-1-carboxylate, m/z (ESI): 509.0 (M+H₂O+H)⁺.

Step 3. (7aS,8R)-2-Chloro-4-(difluoromethyl)-8-(piperazin-1-yl)-5,6,7,7a,8,9-hexahydroazeto[1,2-a]pyrido[3,4-f]azepin-6-ol. To a solution of benzyl 4-(6aS,7R)-2-chloro-4-(difluoromethyl)-4b,5a, 6,6a,7,8-hexahydroazeto[1,2-a]oxireno[2,3-d]pyrido[3,4-f]azepin-7-yl)piperazine-1-carboxylate (400 mg, 0.815 mmol) in EtOAc (10 mL) and EtOH (1 mL) was added palladium 10 wt % on activated carbon (434 mg, 0.407 mmol, Sigma-Aldrich Corporation). The reaction mixture was purged with N₂, followed by H₂ (32 psi), and stirred at rt for 18 h. The reaction mixture was filtered and concentrated to give (7aS,8R)-2-chloro-4-(difluoromethyl)-8-(piperazin-1-yl)-5,6,7,7a,8,9-hexahydroazeto[1,2-a]pyrido[3,4-f]azepin-6-ol, m/z (ESI): 359.0 (M+H)⁺.

Step 4. tert-Butyl 4-((7aS,8R)-2-chloro-4-(difluoromethyl)-6-hydroxy-5,6,7,7a,8,9-hexahydroazeto[1,2-a]pyrido[3,4-f]azepin-8-yl)piperazine-1-carboxylate, Intermediate E9. To solution of (7aS,8R)-2-chloro-4-(difluoromethyl)-8-(piperazin-1-yl)-5,6,7,7a,8,9-hexahydroazeto[1,2-a]pyrido[3,4-f]azepin-6-ol (195 mg, 0.543 mmol) in DCM (2.7 mL), and Et₃N (100 μL, 0.712 mmol) was added (Boc),O (119 mg, 0.543 mmol). The reaction mixture was stirred at rt for 1 h. The reaction was quenched by sat. aq. NH₄Cl solution (3 mL) and extracted with DCM (3×2 mL). The combined organic extracts were washed with brine, dried over Na₂SO₄, and concentrated. The crude material was purified by chromatography, eluting with a gradient of 10%-60% acetone in heptane to give Intermediate E9 (45 mg, 0.098 mmol, 18% yield) m/z (ESI): 459.0 (M+H)⁺.

### Intermediate E10-Benzyl 4-((7aS,8R)-2-chloro-4-(difluoromethyl)-6,6-difluoro-5,6,7,7a,8,9-hexahydroazeto[1,2-a]pyrido[3,4-f]azepin-8-yl)piperazine-1-carboxylate

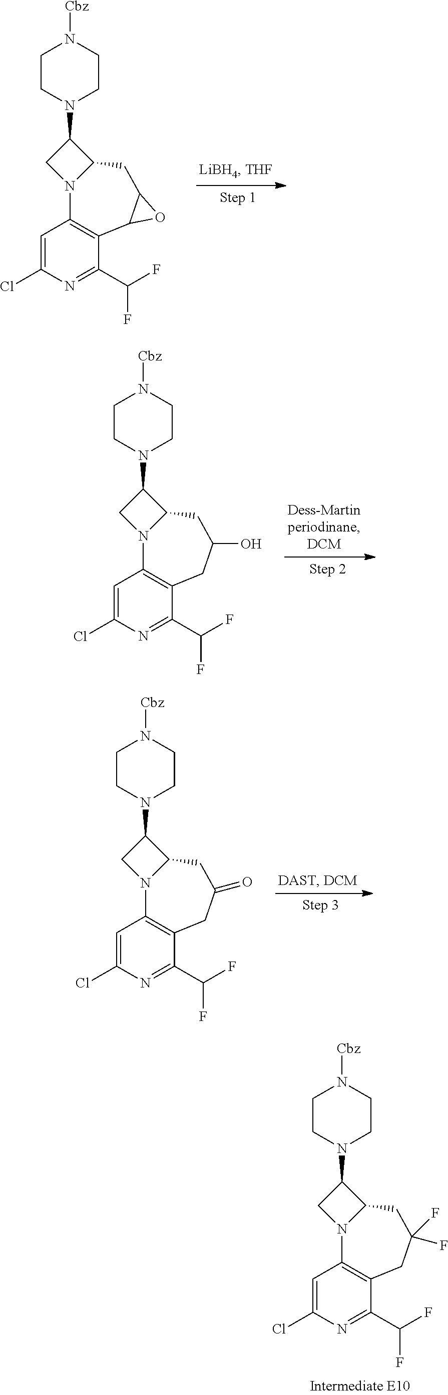

Step 1. Benzyl 4-((7aS,8R)-2-chloro-4-(difluoromethyl)-6-hydroxy-5,6,7,7a,8,9-hexahydroazeto[1,2-a]pyrido[3,4-f]azepin-8-yl)piperazine-1-carboxylate. To a solution of benzyl 4-((6aS,7R)-2-chloro-4-(difluoromethyl)-4b,5a,6,6a,7,8-hexahydroazeto[1,2-a]oxireno[2,3-d]pyrido[3,4-f]azepin-7-yl)piperazine-1-carboxylate (400 mg, 0.815 mmol) in THF (6 mL), was added LiBH; (2.0 M in THE, 0.8 mL, 1.600 mmol, Sigma-Aldrich Corporation) at rt and stirred for 18 h. The reaction was quenched by NH₄Cl sat. aq. solution (2 mL) and extracted with EtOAc (3 mL×3). The combined organic extracts were washed with brine, dried over Na₂SO₄, and concentrated. The crude material was purified by column chromatography, eluting with a gradient of 10%-70% acetone in heptane to give benzyl 4-((7aS,8R)-2-chloro-4-(difluoromethyl)-6-hydroxy-5,6,7,7a,8,9-hexahydroazeto[1,2-a]pyrido[3,4-f]azepin-8-yl)piperazine-1-carboxylate (144 mg, 0.292 mmol, 36% yield), m/z (ESI): 493.0 (M+H)+

Step 2. Benzyl 4-((7aS,8R)-2-chloro-4-(difluoromethyl)-6-oxo-5,6,7,7a,8,9-hexahydroazeto[1,2-a]pyrido[3,4-f]azepin-8-yl)piperazine-1-carboxylate. To a solution of benzyl 4-((7aS,8R)-2-chloro-4-(difluoromethyl)-6-hydroxy-5,6,7,7a,8,9-hexahydroazeto[1,2-a]pyrido[3,4-f]azepin-8-yl)piperazine-1-carboxylate (140 mg, 0.284 mmol) in DCM (2 mL), was added Dess-Martin periodinane (145 mg, 0.341 mmol, Combi-Blocks Inc.). The reaction mixture was stirred at rt for 2 h. The crude material was purified by column chromatography, eluting with a gradient of 10-50% acetone in heptane to give benzyl 4-((7aS,8R)-2-chloro-4-(difluoromethyl)-6-oxo-5,6,7,7a,8,9-hexahydroazeto[1,2-a]pyrido[3,4-f]azepin-8-yl)piperazine-1-carboxylate (131 mg, 0.267 mmol, 94% yield), m/z (ESI): 491.0 (M+H)⁺.

Step 3. Benzyl 4-((7aS,8R)-2-chloro-4-(difluoromethyl)-6,6-difluoro-5,6,7,7a,8,9-hexahydroazeto[1,2-a]pyrido[3,4-f]azepin-8-yl)piperazine-1-carboxylate, Intermediate E10. To a solution of benzyl 4-((7aS,8R)-2-chloro-4-(difluoromethyl)-6-oxo-5,6,7,7a,8,9-hexahydroazeto[1,2-a]pyrido[3,4-f]azepin-8-yl)piperazine-1-carboxylate (130 mg, 0.265 mmol) in DCM (2.5 mL), was added DAST (0.105 mL, 0.794 mmol, Sigma-Aldrich Corporation) at rt. The reaction mixture was stirred at rt for 18 h. The reaction was quenched by NaHCO₃sat. aq. solution (3 mL) and extracted with DCM (3×3 mL). The combined organic extracts were washed with brine, dried over Na₂SO₄, and concentrated to give Intermediate E10 (58 mg, 0.113 mmol, 43% yield), m/z (ESI): 512.8 (M+H)⁺.

### Intermediate E11—Benzyl 4-((6aS,7R)-2-chloro-4-(difluoromethyl)-5-methylene-6,6a, 7,8-tetrahydro-5H-azeto[1,2-a][1,6]naphthyridin-7-yl)piperazine-1-carboxylate

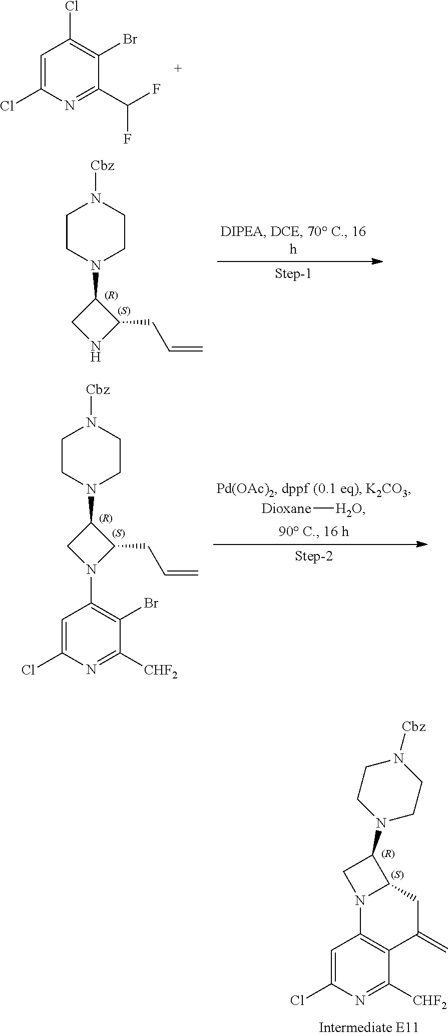

Step 1. Benzyl 4-((28,3R)-2-allyl-1-(3-bromo-6-chloro-2-(difluoromethyl)pyridin-4-yl)azetidin-3-yl)piperazine-1-carboxylate. To a stirred solution of 3-bromo-4,6-dichloro-2-(difluoromethyl)pyridine (10 g, 36.1 mmol) in DCE (60 mL) was added benzyl 4-((25,3R)-2-allylazetidin-3-yl)piperazine-1-carboxylate (17.9 g, 43.3 mmol) followed by DIPEA (63.1 mL, 361 mmol) at 0° C. under N₂ atmosphere. The reaction mixture was stirred at 70° C. for 16 h. The reaction mixture was quenched with water (100 mL) and extracted with DCM (3×50 mL). The combined organic extracts were dried (Na₂SO₄), filtered, and concentrated under reduced pressure. The crude material was purified by chromatography (Redi-Sep pre-packed silica gel column, elution: 0 to 25% EtOAc in hexanes to give benzyl 4-((28,3R)-2-allyl-1-(3-bromo-6-chloro-2-(difluoromethyl)pyridin-4-yl)azetidin-3-yl)piperazine-1-carboxylate (10 g, 50% yield), m/z (ESI): 555.0 (M+H)⁺. 3H NMR (400 MHz, DMSO-d₆): δ 7.35 (tdd, J=8.6, 6.0, 2.3 Hz, 5H), 7.06 (t, J=53.4 Hz, 1H), 6.80 (s, 1H), 5.84 (ddt. J=17.2, 10.0, 7.3 Hz, 1H), 5.21-5.10 (m, 2H), 5.08 (s, 2H), 4.59 (dd, J=9.3, 7.0 Hz, 1H), 4.51 (dd, J=8.0, 4.1 Hz, 1H), 4.04 (q, J=7.1 Hz, 2H), 3.88 (dd, J=9.3, 4.4 Hz, 1H), 3.40 (m, 4H), 2.90 (dt, J=7.3, 4.1 Hz, 1H), 2.49-2.21 (m, 4H).

Step 2. Benzyl 4-((6aS,7R)-2-chloro-4-(difluoromethyl)-5-methylene-6,6a,7,8-tetrahydro-5H-azeto[1,2-a][1,6]naphthyridin-7-yl)piperazine-1-carboxylate (Intermediate Ell). A sealed tube (500 mL) was charged with a solution of benzyl 4-((28,3R)-2-allyl-1-(3-bromo-6-chloro-2-(difluoromethyl)pyridin-4-yl)azetidin-3-yl)piperazine-1-carboxylate (10 g, 17.99 mmol) in 1,4-dioxane (200 mL) and cesium carbonate (14.6 g. 45.0 mmol) at rt. The reaction mixture was degassed and purged with N₂ for 5 min. Then, dppf (0.997 g, 1.799 mmol) and PdOAc₂ (400 mg, 1.799 mmol) were added, and the reaction mixture was stirred at 70° C. for 2 h. The reaction mixture was quenched with water (80 mL) and extracted with EtOAc (3×50 mL). The combined organic extracts were dried (Na₂SO₁), filtered, and concentrated under reduced pressure. The crude material was purified by chromatography (Redi-Sep pre-packed silica gel column, elution: 0 to 45% EtOAc in hexanes) to give benzyl 4-((6aS,7R)-2-chloro-4-(difluoromethyl)-5-methylene-6,6a, 7,8-tetrahydro-5H-azeto[1,2-a][1,6]naphthyridin-7-yl)piperazine-1-carboxylate (5 g, 59% yield), m/z (ESI): 475.2 (M+H)⁺. ¹H NMR (400 MHz, DMSO-d₆): δ 7.45-7.27 (m, 5H), 6.89 (t, J=53.4 Hz, 1H), 6.60 (s, 1H), 5.38 (s, 1H), 5.26 (s, 1H), 5.09 (s, 2H), 4.27 (dt, J=11.4, 5.5 Hz, 1H), 4.16 (dd, J=9.4, 6.2 Hz, 1H), 3.96 (t, J=8.7 Hz, 1H), 3.45 (s, 4H), 3.24-3.14 (m, 1H), 2.77 (dd, J=11.9, 5.7 Hz, 1H), 2.32 (dd, J=13.5, 8.5 Hz, 5H).

Intermediates in Table 1-14 were prepared following the procedure described for Intermediate E11, using appropriate starting materials. All starting materials are commercially available or are described above.

TABLE 1-14

| | Chemical Structure & Name | LCMS: (ESI + ve ion) m/z; NMR | Comments |
|---|---|---|---|
| E12 | 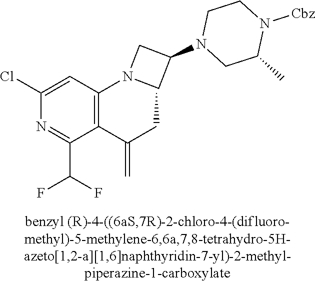 | m/z (ESI): 489.3 (M + H)⁺, 'H NMR (400 MHz, DMSO-d₆) δ 7.43-7.28 (m, 5H), 6.89 (t, J = 53.4 Hz, 1H), 6.60 (s, 1H), 5.39 (s, 1H), 5.26 (s, 1H), 5.16-5.02 (m, 2H), 4.25 (ddt, J = 14.6, 10.7, 5.3 Hz, 2H), 4.10 (dd, J = 9.4, 6.1 Hz, 1H), 3.96 (t, J = 8.7 Hz, 1H), 3.82 (d, J = 13.1 Hz, 1H), 3.20-3.05 (m, 2H), 2.84 (d, J = 10.9 Hz, 1H), 2.77 (dd, J = 11.9, 5.7 Hz, 1H), 2.56 (d, J = 11.1 Hz, 1H), 2.29 (t, J = 11.8 Hz, 1H), 2.09- 1.99 (m, 1H), 1.89 (td, J = 11.5, 3.4 Hz, 1H), 1.22 (d, J = 6.7 Hz, 3H). | |

### Intermediate E13-8-(4-Benzylpiperazin-1-yl)-2-chloro-4-(trifluoromethyl)-7,7a,8,9-tetrahydro-5H-azeto[2,1-c]pyrido[4,3-e][1,4]oxazepine

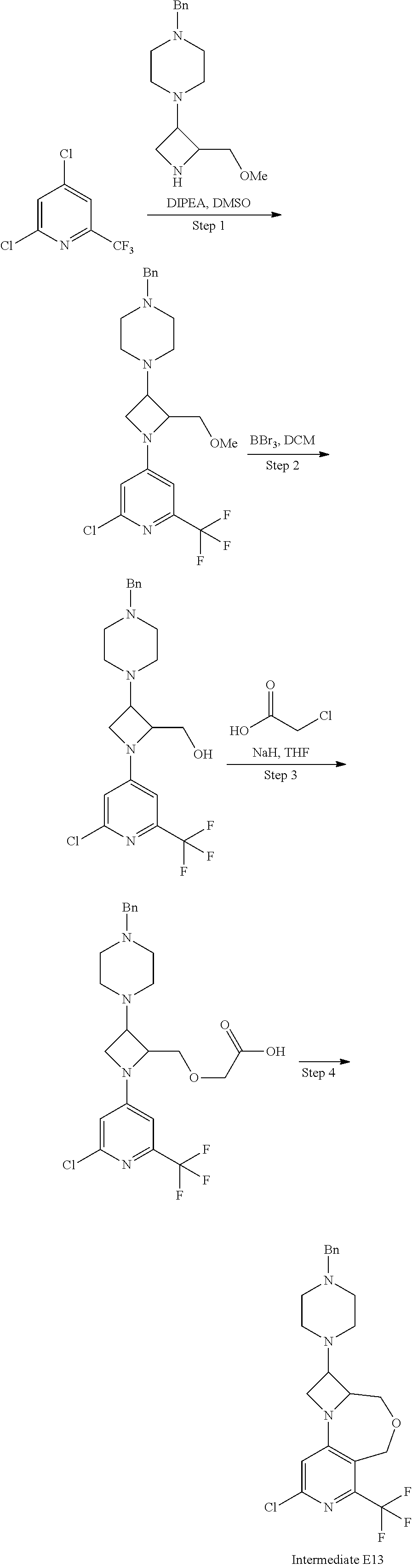

Step 1. 1-Benzyl-4-(1-(2-chloro-6-(trifluoromethyl)pyridin-4-yl)-2-(methoxymethyl)azetidin-3-yl)piperazine. To a solution of 1-benzyl-4-(2-(methoxymethyl)azetidin-3-yl)piperazine (975 mg, 3.54 mmol) and 2,4-dichloro-6-(trifluoromethyl)pyridine (765 mg, 3.54 mmol, Oakwood Products, Inc.) in DMSO (10 mL) was added DIPEA (4.33 mL, 24.7 mmol). The reaction mixture was stirred at rt for 18 h. The reaction was quenched by NaHCO₃ sat. aq. solution (60 mL) and extracted with EtOAc (60 ml×3). The combined organic extracts were washed with brine, dried over Na₂SO₄, and concentrated. The crude material was purified by column chromatography, eluting with a gradient of 0%-100% EtOAc in heptane to give 1-benzyl-4-(1-(2-chloro-6-(trifluoromethyl)pyridin-4-yl)-2-(methoxymethyl)azetidin-3-yl)piperazine (385 mg, 0.85 mmol, 24% yield), m/z (ESI): 455.2 (M+H)⁺.

Step 2. (3-(4-Benzylpiperazin-1-yl)-1-(2-chloro-6-(trifluoromethyl)pyridin-4-yl)azetidin-2-yl) methanol. To a solution of 1-benzyl-4-(1-(2-chloro-6-(trifluoromethyl)pyridin-4-yl)-2-(methoxymethyl)azetidin-3-yl)piperazine (385 mg, 0.85 mmol) in DCM (4 mL) at 0° C., was added boron tribromide (1 M solution in DCM, 3.4 mL, 3.4 mmol). The reaction mixture was allowed to warm to rt and stir for 45 min. The reaction was quenched by NaHCO₃ sat. aq. solution (60 mL.) and extracted with DCM (20 mL×3). The combined organic extracts were washed with brine, dried over Na₂SO₄, and concentrated. The crude material was purified by column chromatography, eluting with a gradient of 0%-100% EtOAc/EtOH (3:1) in heptane to give (3-(4-benzylpiperazin-1-yl)-1-(2-chloro-6-(trifluoromethyl)pyridin-4-yl)azetidin-2-yl) methanol (208 mg, 0.47 mmol, 56% yield), m/z (ESI): 441.1 (M+H)⁺.

Step 3. 2-((3-(4-Benzylpiperazin-1-yl)-1-(2-chloro-6-(trifluoromethyl)pyridin-4-yl)azetidin-2-yl)methoxy) acetic acid. To a solution of (3-(4-benzylpiperazin-1-yl)-1-(2-chloro-6-(trifluoromethyl)pyridin-4-yl)azetidin-2-yl) methanol (203 mg, 0.46 mmol) and chloroacetic acid (43 mg, 0.46 mmol) in THF (2 mL) was added NaH (55 mg, 1.38 mmol, 60% in mineral oil). The reaction mixture was stirred at rt for 2 h. Additional chloroacetic acid (43 mg, 0.46 mmol) and NaH (55 mg, 1.38 mmol, 60% in mineral oil) were added and the reaction mixture was stirred at rt for 3 days. The reaction mixture was quenched with MeOH (20 mL) and concentrated under vacuum.

Chromatographic purification of the crude (silica gel, 0-15% 2M NH₃ in MeOH in DCM) gave 2-((3-(4-benzylpiperazin-1-yl)-1-(2-chloro-6-(trifluoromethyl)pyridin-4-yl)azetidin-2-yl)methoxy) acetic acid (98 mg, 0.20 mmol, 43% yield), m/z (ESI): 498.9 (M+H)⁺.

Step 4. 8-(4-Benzylpiperazin-1-yl) ˜2-chloro-4-(trifluoromethyl)-7,7a,8,9-tetrahydro-5H-azeto[2,1-c]pyrido[4,3-c][1,4]oxazepine, Intermediate E13. To a solution of gave 2-((3-(4-benzylpiperazin-1-yl)-1-(2-chloro-6-(trifluoromethyl)pyridin-4-yl)azetidin-2-yl)methoxy) acetic acid (98 mg, 0.2 mmol) and N-hydroxyphthalimide (35 mg, 0.22 mmol) in EtOAc (2 mL) was added DIC (0.036 mL, 0.24 mmol), and the mixture was stirred at rt for 15 min. The reaction mixture was concentrated under vacuum, and the residue was redissolved in DMSO (8 mL). The reaction mixture was placed under N₂ atmosphere, and 4CZIPN (3 mg, 3.9 mmol, Sigma-Aldrich) followed by TFA (0.3 mL, 3.92 mmol) were added. The mixture was irradiated with 420 nm light while stirring for 1 h. The reaction was quenched by NaHCO₃sat. aq. solution (10 mL) and extracted with EtOAc (10 mL×3). The combined organic extracts were washed with brine, dried over Na₂SO₄, and concentrated. The crude material was purified by column chromatography, eluting with a gradient of 0%-100% EtOAc/EtOH (3:1) in heptane to give 8-(4-benzylpiperazin-1-yl)-2-chloro-4-(trifluoromethyl)-7.7a,8,9-tetrahydro-5H-azeto[2,1-c]pyrido[4,3-e][1,4]oxazepine (21 mg, 0.046 mmol, 23% yield), m/z (ESI): 453.3 (M+H)⁺, as a mixture of regioisomers.

### Intermediate F1—2-2(4-(1-(2-Methoxyethyl)-4-methyl-1H-pyrazol-5-yl)piperidin-1-yl)-4-(methylsulfonyl)-6-(trifluoromethyl)-5-vinylpyrimidine

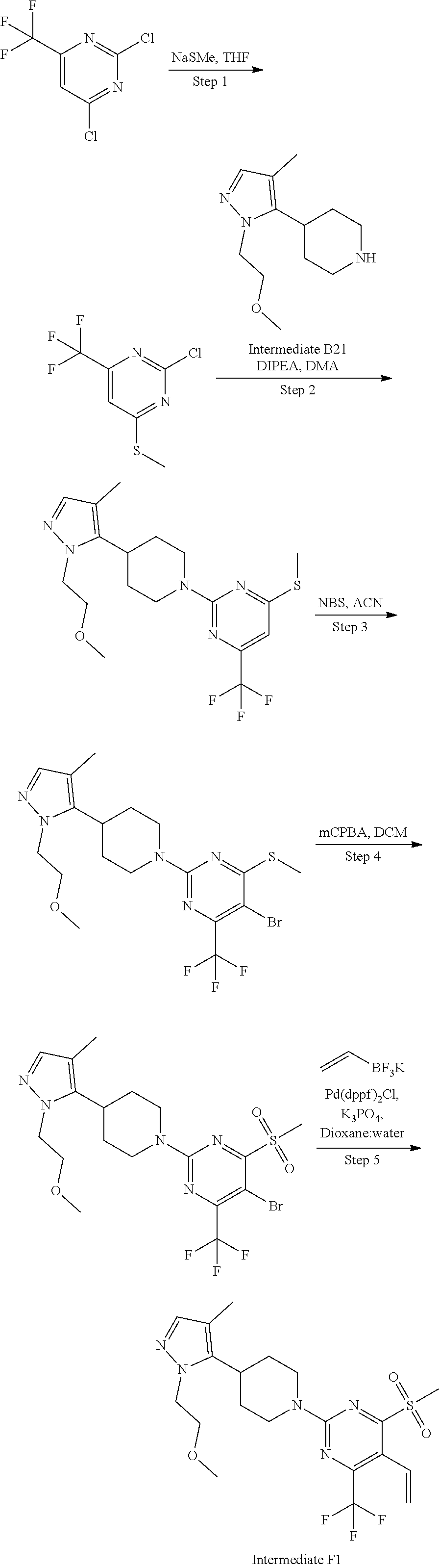

Step 1. 2-Chloro-4-(methylthio)-6-(trifluoromethyl)pyrimidine. To a stirred solution of 2,4-dichloro-6-(trifluoromethyl)pyrimidine (30 g, 138 mmol) under N₂ atmosphere in THF (300 mL) was added a solution of sodium thiomethoxide (10.66 g. 152 mmol) in water (10 mL) at −10° C., and the reaction was stirred for 3 h. The reaction mixture was diluted with water (200 mL) and extracted with EtOAc (2×100 mL). The combined organic extracts were dried over Na₂SO₄, filtered, concentrated, and purified by column chromatography, eluting with a gradient of 0-100% EtOAc in pet. ether, to provide 2-chloro-4-(methylthio)-6-(trifluoromethyl)pyrimidine (25 g, 109 mmol, 79% yield), m/z (ESI): 229.1 (M+H)⁺.

Step 2. 2-(4-(1-(2-Methoxyethyl)-4-methyl-1H-pyrazol-5-yl)piperidin-1-yl)-4-(methylthio)-6-(trifluoromethyl)pyrimidine. To a solution of Intermediate B21, TFA salt (7 g. 21.85 mmol) in DMA (35.0 mL), was added 2-chloro-4-(methylthio)-6-(trifluoromethyl)pyrimidine (5.00 g, 21.85 mmol) and DIPEA (19.08 mL, 109 mmol), and the reaction mixture was heated to 100° C. for 4 h. The reaction was concentrated and purified by column chromatography, eluting with a gradient of 0% to 100% EtOAc in pet. ether, to provide 2-(4-(1-(2-methoxyethyl)-4-methyl-1H-pyrazol-5-yl)piperidin-1-yl)-4-(methylthio)-6-(trifluoromethyl)pyrimidine (7 g, 16.85 mmol, 77% yield), m/z (ESI): 416.3 (M+H)⁺.

Step 3. 5-Bromo-2-(4-(1-(2-methoxyethyl)-4-methyl-1H-pyrazol-5-yl)piperidin-1-yl)-4-(methylthio)-6-(trifluoromethyl)pyrimidine. To a solution of 2-(4-(1-(2-methoxyethyl)-4-methyl-1H-pyrazol-5-yl)piperidin-1-yl)-4-(methylthio)-6-(trifluoromethyl)pyrimidine (1.9 g, 4.57 mmol) in ACN (38 mL) was added NBS (0.81 g, 4.57 mmol), and the reaction mixture was stirred at rt for 3 h. The reaction was concentrated and purified by column chromatography, eluting with a gradient of 0% to 30% EtOAc in hexanes, to provide 5-bromo-2-(4-(1-(2-methoxyethyl)-4-methyl-1H-pyrazol-5-yl)piperidin-1-yl)-4-(methylthio)-6-(trifluoromethyl)pyrimidine (2.0 g, 4.05 mmol, 88% yield), m/z (ESI): 494.0 and 495.9 (M+H)⁺.

Step 4. 5-Bromo-2-(4-(1-(2-methoxyethyl)-4-methyl-1H-pyrazol-5-yl)piperidin-1-yl)-4-(methylsulfonyl)-6-(trifluoromethyl)pyrimidine. To a solution of 5-bromo-2-(4-(1-(2-methoxyethyl)-4-methyl-1H-pyrazol-5-yl)piperidin-1-yl)-4-(methylthio)-6-(trifluoromethyl)pyrimidine (8 g, 16.18 mmol) in DCM (160 mL) was added m-CPBA (10.74 g, 40.5 mmol) portion wise at 0° C. The reaction mixture was warmed to rt and stirred for 30 min. Then, the reaction mixture was cooled to 0° C., and additional m-CPBA (4.30 g, 16.18 mmol) was added portion wise. The reaction mixture was warmed up to rt and stirred for another 1.5 h. The reaction mixture was quenched with satd. sodium metabisulfite solution (50 mL), and the biphasic mixture was separated. The aqueous solution was extracted with DCM (2×70 mL), the combined organic extracts was washed with NaHCO₃ (3×200 mL), followed by brine (1×200 mL). The combined organic extracts were dried over Na₂SO₄, filtered, and concentrated. The crude material was purified by column chromatography, eluting with a gradient of 20-25% EtOAc/pet. ether to provide 5-bromo-2-(4-(1-(2-methoxyethyl)-4-methyl-1H-pyrazol-5-yl)piperidin-1-yl)-4-(methylsulfonyl)-6-(trifluoromethyl)pyrimidine (4.2 g, 7.98 mmol, 49% yield), m/z (ESI): 526.0 and 528.0 (M+H)⁺.

Step 5. 2-(4-(1-(2-Methoxyethyl)-4-methyl-1H-pyrazol-5-yl)piperidin-1-yl)-4-(methylsulfonyl)-6-(trifluoromethyl)-5-vinylpyrimidine, Intermediate F1. A stirred solution of 5-bromo-2-(4-(1-(2-methoxyethyl)-4-methyl-1H-pyrazol-5-yl)piperidin-1-yl)-4-(methylsulfonyl)-6-(trifluoromethyl)pyrimidine (4.2 g, 7.98 mmol), potassium trifluoro (vinyl) borate (2.14 g, 15.96 mmol), K₃PO₄ (5.08 g, 23.94 mmol) in 1,4-dioxane (76 mL) and water (8.40 mL) was purged with N₂ for 5 min. Then, Pd(dppf)Cl₂-DCM adduct (0.326 g. 0.40 mmol) was added, and the resulting reaction mixture was heated to 90° C. for 24 h. Then, the reaction mixture was cooled to rt, diluted with EtOAc (200 mL) and washed with brine (1×150 mL). The organic extracts was dried over Na₂SO₄, filtered, concentrated, and purified by column chromatography, eluting with a gradient of 50-65% EtOAc/Pet. ether to provide Intermediate F1 (0.65 g, 1.37 mmol, 17% yield), m/z (ESI): 474.0 (M+H)⁺. ¹H NMR (400 MHz, CDCl₃) δ (ppm) 7.27 (s, 1H), 6.85 (ddd, J=17.7, 11.4, 1.7 Hz, 1H), 5.73 (dd, J=11.5, 1.1 Hz, 1H), 5.62 (d, J=17.7 Hz, 1H), 4.92 (br s, 2H), 4.30 (t, J=5.2 Hz, 2H), 3.74 (t, J=5.2 Hz, 2H), 3.33 (d, J=1.9 Hz, 6H), 3.16-2.98 (m, 3H), 2.10 (d, J=2.7 Hz, 3H), 2.03-1.90 (m, 4H).

Intermediates in Table 1-15 were prepared following the procedure described for Intermediate F1, using appropriate starting materials. All starting materials are commercially available or are described above.

TABLE 1-15

| | Chemical Structure & Name | LCMS: (ESI + ve ion) m/z: NMR | Comments |
|---|---|---|---|
| F2 | 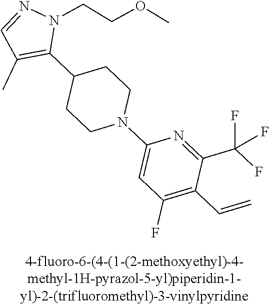 [SMILES: C=Cc1c(F)cc(N2CCC(c3c(C)cnn3CCOC)CC2)nc1C(F)(F)F] | m/z (ESI): 413.3 (M + H)⁺. ¹H NMR (DMSO-d₆, 400 MHz): δ (ppm) 7.69 (s, 1H), 7.13 (t, J = 7.2 Hz, 2H), 6.60 (ddd, J = 17.7, 11.6, 2.5 Hz, 1H), 5.92 (m, 1H), 5.71 (m, 1H), 5.56 (dt, J = 11.7, 2.0 Hz, 1H), 4.49 (d, J = 13.1 Hz, 2H), 4.23 (t, J = 5.4 Hz, 2H), 3.61 (t, J = 5.4 Hz, 2H), 3.22 (s, 3H), 3.14 (dq, J = 11.1, 6.1, 5.6 Hz, 1H), 2.98 (m, 2H), 1.97 (s, 3H), 1.79 (m, 4H). | Steps 1 and 3 were omitted; Step 2. Intermediate A20 was used; Step 4. TBAF was used SFC was performed after Step 2 (1^{st} eluting isomer) |
| F3 | 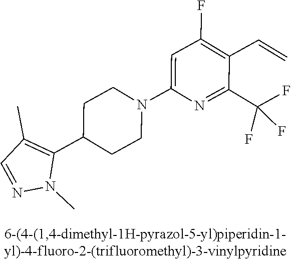 [SMILES: C=Cc1c(F)cc(N2CCC(c3c(C)cnn3C)CC2)nc1C(F)(F)F] | m/z (ESI): 369.3 (M + H)⁺. ¹H NMR (400 MHz, DMSO-d₆): δ (ppm) 7.13 (d, J] = 14.5 Hz, 1H), 7.07 (s, 1H), 6.60 (ddt, J = 17.1, 11.7, 2.6 Hz, 1H), 5.87-5.65 (m, 1H), 5.55 (dt, J = 11.8, 1.9 Hz, 1H), 4.50 (d, J = 13.2 Hz, 2H), 3.77 (s, 3H), 3.17-2.86 (m, 3H), 1.97 (s, 3H), 1.81 (dd, J = 8.1, 3.3 Hz, 4H). | Steps 1 and 3 were omitted; Step 2. Intermediates A20 and B20 were used; Step 4. TBAF was used |
| F4 | 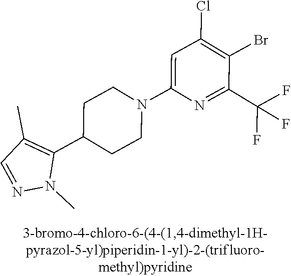 [SMILES: Cc1cnn(C)c1C1CCN(c2cc(Cl)c(Br)c(C(F)(F)F)n2)CC1] | m/z (ESI): 436.6 and 438.9 (M + H)⁺. ¹H NMR (400 MHz, DMSO-d₆): δ (ppm) 7.51 (s, 1H), 7.07 (s, 1H), 4.49 (d, J = 13.1 Hz, 2H), 3.78 (s, 3H), 3.20-3.04 (m, 1H), 2.99 (dt, J = 14.6, 7.1 Hz, 2H), 1.97 (s, 3H), 1.80 (tt, J = 10.0, 4.0 Hz, 4H). | Steps 1 and 4 were omitted; Step 2. Intermediates A20 and B20 were used; |

### Section 2: Synthesis of Example Compounds

### Method 1

### Example 1:1-(4-((7aS,8R)-4-(Difluoromethyl)-2-(4-(2-(3-methoxy-3-oxetanyl)-4-methyl-3-pyridinyl)-1-piperidinyl)-7,7a,8,9-tetrahydroazeto[1,2-a]pyrido[3,4-f]azepin-8-yl)-1-piperazinyl)-2-propen-1-one

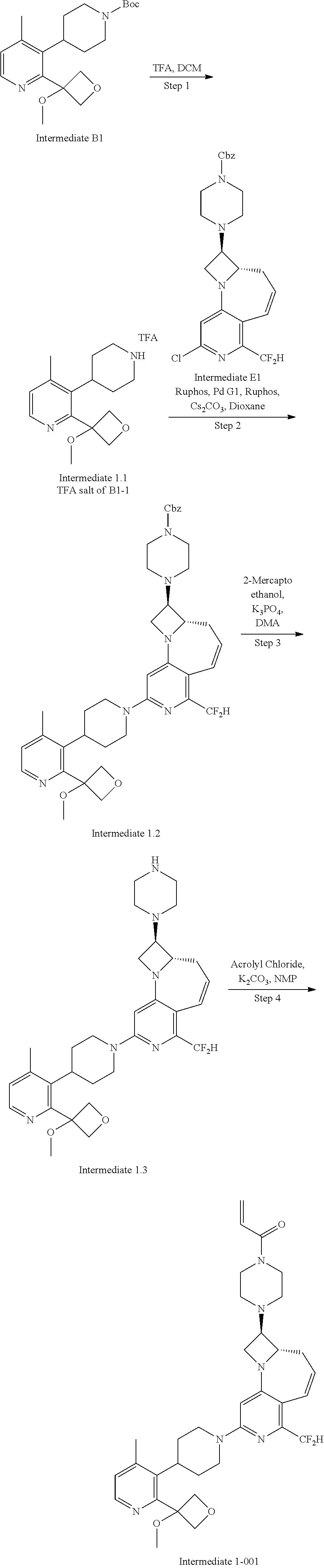 [SMILES: COC1(c2nccc(C)c2C2CCN(C(=O)OC(C)(C)C)CC2)COC1 ; COC1(c2nccc(C)c2C2CCNCC2)COC1 ; O=C(OCc1ccccc1)N1CCN([C@@H]2CN3c4cc(Cl)nc(C(F)F)c4C=CC[C@@H]23)CC1 ; COC1(c2nccc(C)c2C2CCN(c3cc4c(c(C(F)F)n3)C=CC[C@H]3[C@H](N5CCN(C(=O)OCc6ccccc6)CC5)CN43)CC2)COC1 ; COC1(c2nccc(C)c2C2CCN(c3cc4c(c(C(F)F)n3)C=CC[C@H]3[C@H](N5CCNCC5)CN43)CC2)COC1 ; C=CC(=O)N1CCN([C@@H]2CN3c4cc(N5CCC(c6c(C)ccnc6C6(OC)COC6)CC5)nc(C(F)F)c4C=CC[C@@H]23)CC1]

Step 1. 2-(3-Methoxyoxetan-3-yl)-4-methyl-3-(piperidin-4-yl)pyridine trifluoroacetate, Intermediate 1.1. To a stirred solution of Intermediate B1 (85 g, 235 mmol) in DCM (850 mL) at 0° C., was added TFA (250 mL, 3.25 mol), and the mixture was stirred for 3 b at rt. The reaction mixture was concentrated, co-evaporated with toluene (3×150 mL), triturated with diethyl ether (4×150 mL), and concentrated to afford Intermediate 1.1 (85 g, 236 mmol, 100% yield), m/z (ESI): 263.2 (M+H)⁺.

Step 2. Benzyl 4-((7aS,8R)-4-(difluoromethyl)-2-(4-(2-(3-methoxyoxetan-3-yl)-4-methylpyridin-3-yl)piperidin-1-yl)-7,7a,8,9-tetrahydroazeto[1,2-a]pyrido[3,4-f]azepin-8-yl)piperazine-1-carboxylate, Intermediate 1.2. To a stirred solution of Intermediate E1 (54.5 g, 152 mmol) in 1,4-dioxane (1.800 L) at 0° C., was added Cs₂CO₃ (823 g. 2.53 mol) portion wise and stirred for 10 min. To this reaction mixture, Intermediate 1.1 (60 g, 126 mmol), RuPhos (5.9 g. 12.63 mmol), and RuPhos Pd G1 (10.3 g, 12.63 mmol) were added sequentially, and the reaction mixture was degassed, purged with N₂ for 10 min and stirred at 80° C. for 2 h. The reaction mixture was quenched with water (1000 mL) and extracted with EtOAc (2×1000 mL). The combined extracts were washed with water (2×2000 mL) followed by brine (2×1000 mL), dried over Na₂SO₄, filtered, and concentrated. The crude material was purified by flash chromatography eluting with 60 to 100% EtOAc in hexanes to give Intermediate 1.2 (60 g, 86 mmol, 68% yield), m/z (ESI): 701.4 (M+H)⁺. ¹H NMR (400 MHz, DMSO-d₆): δ (ppm) 8.29 (d, J=4.9 Hz, 1H), 7.46-7.25 (m, 5H), 7.17 (d, J=5.0 Hz, 1H), 6.78 (t, J=54.4, 1H), 6.50-6.30 (m, 1H), 5.79 (s, 1H), 5.71 (dt, J=12.6, 4.8 Hz, 1H), 5.15 (d, J=7.1 Hz, 2H), 5.08 (s, 2H), 4.81 (d, J=7.1 Hz, 2H), 4.46 (t, J=13.4 Hz, 2H), 3.91 (t, J=7.4 Hz, 1H), 3.78 (q, J=6.1 Hz, 1H), 3.67 (t, J=7.4 Hz, 1H), 3.41 (br s, 4H), 3.05-3.00 (m, 1H), 2.95 (s, 3H), 2.75 (q, J=14.8, 14.0 Hz, 3H), 2.67-2.57 (m, 2H), 2.40 (s, 3H), 2.39-2.26 (m, 4H), 2.07 (td, J=13.2, 6.6 Hz, 2H), 1.59 (d, J=12.5 Hz, 2H).

Step 3. (7aS,8R)-4-(Difluoromethyl)-2-(4-(2-(3-methoxyoxetan-3-yl)-4-methylpyridin-3-yl)piperidin-1-yl)-8-(piperazin-1-yl)-7, 7a,8,9-tetrahydroazeto[1,2-a]pyrido[3,4-f]azepine, Intermediate 1.3. To a stirred solution of Intermediate 1.2 (48 g, 68.5 mmol) in DMA (500 mL) were added K₃PO₄ (43.6 g, 205 mmol) and 2-mercaptoethanol (12.16 mL, 171 mmol), and the reaction mixture was stirred at 95° C. for 24 h. The reaction mixture was quenched with ice water (1000 mL) and extracted with EtOAc (2×750 mL). The combined organic extracts were washed with water (3×1000 mL), followed by brine (1500 mL), dried with Na₂SO₄, filtered, and concentrated. The crude material was purified by flash chromatography eluting with 4 to 7% MeOH in DCM to give Intermediate 1.3, m/z (ESI): 567.2 (M+H)⁺. JH NMR (400 MHz, DMSO-d₆): δ (ppm) 8.29 (d, J=4.9 Hz, 1H), 7.17 (d, J=5.0 Hz, 1H), 7.00-6.44 (m, 2H), 5.79 (s, 1H), 5.71 (ddd, J=12.6, 5.3, 4.0 Hz, 1H), 5.15 (d, J=7.1 Hz, 2H), 4.81 (d, J=7.1 Hz, 2H), 4.46 (t, J=13.8 Hz, 2H), 3.89 (t, J=7.4 Hz, 1H), 3.75 (q, J=6.2 Hz, 1H), 3.65 (t, J=7.4 Hz, 1H), 3.18 (s, 1H), 2.96 (s, 3H), 2.94-2.85 (m, 1H). 2.89-2.70 (m, 7H), 2.66-2.58 (m, 2H), 2.38 (s, 3H), 2.25 (s, 4H), 2.16-1.98 (m, 2H), 1.66-1.50 (m, 2H).

Step 4. 1-(4-((7aS,8R)-4-(Difluoromethyl)-2-(4-(2-(3-methoxy-3-oxetanyl)-4-methyl-3-pyridinyl)-1-piperidinyl)-7,7a,8,9-tetrahydroazeto[1,2-a]pyrido[3,4-f]azepin-8-yl)-1-piperazinyl)-2-propen-1-one. Compound 1-001. To a solution of Intermediate 1.3 (28 g, 49.4 mmol) in NMP (280 mL) at −15° C. was added K₂CO₃ (137 g, 988 mmol) and stirred for 15 min. A solution of acryloyl chloride (4.82 mL, 59.3 mmol) in NMP (5 mL) was added dropwise over 5 min and stirred for 15 min. The reaction mixture was slowly quenched into ice cold water (1000 mL) and extracted with EtOAc (2×750 mL). The combined organic extracts were washed with water (4×1000 mL) followed by brine (1000 mL), dried with Na₂SO₄, filtered, and concentrated. The crude material was purified by flash chromatography eluting with 5 to 10% IPA in EtOAc to give (1-(4-((7aS,8R)-4-(Difluoromethyl)-2-(4-(2-(3-methoxy-3-oxetanyl)-4-methyl-3-pyridinyl)-1-piperidinyl)-7,7a,8,9-tetrahydroazeto[1,2-a]pyrido[3,4-f]azepin-8-yl)-1-piperazinyl)-2-propen-1-one, Compound 1-001 (22 g. 35.4 mmol, 72% yield), m/z (ESI): 621.2 (M+H)⁺. ¹H NMR (400 MHz, DMSO-d₆): δ (ppm) 8.29 (d, J=4.9 Hz, 1H), 7.17 (d, J=5.0 Hz, 1H), 7.03-6.52 (m, 3H), 6.11 (dd, J=16.7, 2.4 Hz, 1H), 5.77 (d, J=22.6 Hz, 1H), 5.77-5.59 (m, 2H), 5.15 (d, J=7.1 Hz, 2H), 4.82 (d, J=7.1 Hz, 2H), 4.58-4.27 (m, 2H), 3.92 (t, J=7.4 Hz, 1H), 3.80 (q, J=6.1 Hz, 1H), 3.70 (t, J=7.4 Hz, 1H), 3.65-3.47 (m, 4H), 3.05-2.90 (m, 4H), 2.84-2.69 (m, 3H), 2.69-2.56 (m, 2H), 2.42-2.28 (m, 7H), 2.06 (ddt, J=18.2, 12.3, 6.2 Hz, 2H), 1.59 (d, J=12.6 Hz, 2H).

### Example 2:1-(4-((7aS,8R)-4-(Difluoromethyl)-2-(4-(1-(1-methoxycyclopropyl)-4-methyl-1H-pyrazol-5-yl)-1-piperidinyl)-7,7a,8,9-tetrahydroazeto[1,2-a]pyrido[3,4-f]azepin-8-yl)-1-piperazinyl)-2-propon-1-one

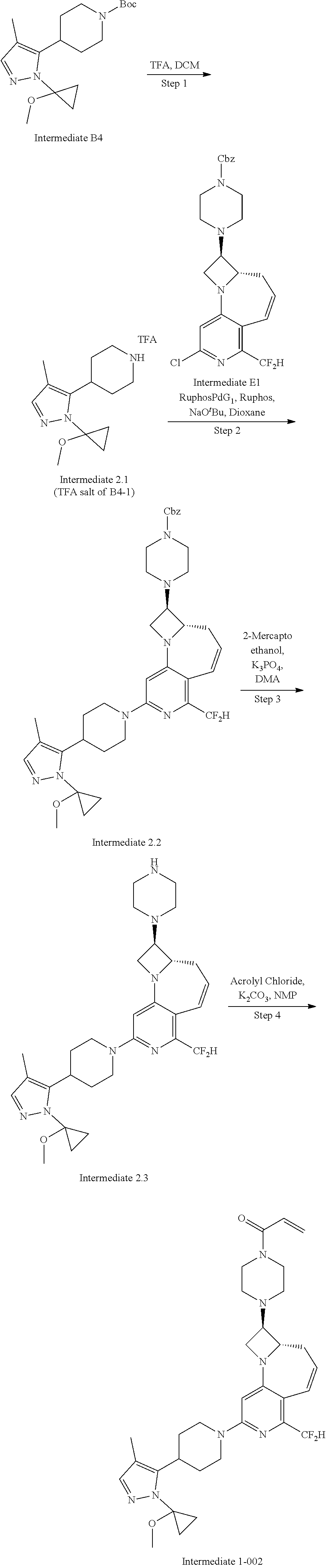 [SMILES: COC1(n2ncc(C)c2C2CCN(C(=O)OC(C)(C)C)CC2)CC1 ; COC1(n2ncc(C)c2C2CCNCC2)CC1 ; O=C(OCc1ccccc1)N1CCN([C@@H]2CN3c4cc(Cl)nc(C(F)F)c4C=CC[C@@H]23)CC1 ; COC1(n2ncc(C)c2C2CCN(c3cc4c(c(C(F)F)n3)C=CC[C@H]3[C@H](N5CCN(C(=O)OCc6ccccc6)CC5)CN43)CC2)CC1 ; COC1(n2ncc(C)c2C2CCN(c3cc4c(c(C(F)F)n3)C=CC[C@H]3[C@H](N5CCNCC5)CN43)CC2)CC1 ; C=CC(=O)N1CCN([C@@H]2CN3c4cc(N5CCC(c6c(C)cnn6C6(OC)CC6)CC5)nc(C(F)F)c4C=CC[C@@H]23)CC1]

Step 1. 4-(1-(1-Methoxycyclopropyl)-4-methyl-1H-pyrazol-5-yl)piperidine, Intermediate 2.1. To a solution of Intermediate B4 (380 mg, 1.133 mmol) in DCM (2.0 mL) was added TEA (25 mL, 3.35 mmol), and the reaction mixture was stirred at rt for 30 min. The mixture was concentrated to give Intermediate 2.1 as a TFA salt, m/z (ESI): 236.2 (M+H)⁺.

Step 2. Benzyl 4-((7aS,8R)-4-(difluoromethyl)-2-(4-(1-(1-methoxycyclopropyl)-4-methyl-1H-pyrazol-5-yl)piperidin-1-yl)-7,7a,8,9-tetrahydroazeto[1,2-a]pyrido[3,4-f]azepin-8-yl)piperazine-1-carboxylate, Intermediate 2.2. The crude product from Step 1 was dissolved in THF (7.0 mL), and sodium tert-butoxide (1089 mg, 11.33 mmol, Sigma-Aldrich Corporation), RuPhos Pd G3 (95 mg, 0.113 mmol, AstaTech, Inc), and Intermediate E1 (538 mg, 1.133 mmol) in THF (7.0 mL) were added. The reaction mixture was degassed with N₂ for 15 min, then heated to 80° C., and stirred for 4 h. The reaction mixture was diluted with sat. aq. NH₄Cl and EtOAc, and the aqueous layer was extracted with EtOAc. The combined organic extracts were washed with brine, dried over Na₂SO₄, filtered, and concentrated. The crude material was purified by chromatography, eluting with a gradient of 0% to 60% EtOAc in heptane, to provide Intermediate 2.2 (466 mg, 0.692 mmol, 61% yield), m/z (ESI): 674.0 (M+H)⁺.

Step 3. (7aS,8R)-4-(Difluoromethyl)-2-(4-(1-(1-methoxycyclopropyl)-4-methyl-1H-pyrazol-5-yl)piperidin-1-yl)-8-(piperazin-1-yl)-7,7a,8,9-tetrahydroazeto[1,2-a]pyrido[3,4-f]azepine, Intermediate 2.3. Intermediate 2.2 (0.356 g, 0.528 mmol), K₃PO₄ (0.449 g, 2.113 mmol, Combi-Blocks Inc.), DMA (3 mL), and 2-mercaptoethanol (0.074 mL, 1.057 mmol, Sigma-Aldrich Corporation) were heated to 95° C., and stirred for 24 h. The reaction mixture was diluted with water and extracted with EtOAc. The organic extracts were washed with brine, dried over Na₂SO₄, filtered, and concentrated. The crude material was purified by chromatography, eluting with a gradient of 0% to 20% MeOH in DCM, to provide Intermediate 2.3 (0.185 g, 0.343 mmol, 65% yield), m/z (ESI): 540.0 (M+H).

Stop 4. 1-(4-((7aS,8R)-4-(difluoromethyl)-2-(4-(1-(1-methoxycyclopropyl)-4-methyl-1H-pyrazol-5-yl)piperidin-1-yl)-7,7a,8,9-tetrahydroazeto[1,2-a]pyrido[3,4-f]azepin-8-yl)piperazin-1-yl)prop-2-en-1-one, Compound 1-002. To a solution of Intermediate 2.3 (0.1853 g, 0.343 mmol) in DCM (3 mL) was added DIPEA (0.180 mL, 1.030 mmol) and acryloyl chloride (0.2 M in DCM) (1.72 mL, 0.343 mmol, Sigma-Aldrich Corporation), and the reaction mixture was stirred at rt for 30 min. The reaction mixture was concentrated, redissolved in EtOAc, and washed with sat. aq. NH₄Cl. The aqueous phase was extracted with EtOAc, and the organic extracts were washed with brine, dried with Na₂SO₄, and concentrated. The crude material was purified by chromatography, eluting with a gradient of 0% to 80% EtOAc/EtOH (3:1) in heptane, to provide 1-(4-((7aS,8R)-4-(difluoromethyl)-2-(4-(1-(1-methoxycyclopropyl)-4-methyl-1H-pyrazol-5-yl)piperidin-1-yl)-7,7a,8,9-tetrahydroazeto[1,2-a]pyrido[3,4-f]azepin-8-yl)piperazin-1-yl)prop-2-en-1-one, Compound 1-002 (0.158 g, 0.266 mmol, 77% yield), m/z (ESI): 594.0 (M+H)⁺, ¹H NMR (CDC), 400 MHz) δ (ppm) 7.19-7.22 (m, 1H), 6.85 (br d, J=12.2 Hz, 1H). 6.39-6.72 (m, 2H), 6.30 (dd, J=16.9, 1.8 Hz, 1H), 5.69-5.77 (m, 2H), 5.61 (s, 1H), 4.46 (br dd, J=26.0, 13.0 Hz, 2H), 3.92-4.00 (m, 2H), 3.54-3.81 (m, 5H), 3.37-3.49 (m, 1H), 3.20 (s, 3H), 2.99 (q, J=6.6 Hz, 1H), 2.80-2.92 (m, 2H), 2.68-2.78 (m, 1H), 2.58-2.66 (m, 1H), 2.31-2.51 (m, 4H), 2.06-2.08 (m, 3H), 1.95-2.05 (m, 2H), 1.80 ˜ 1.86 (m, 2H), 1.36 (br d, J=3.5 Hz, 4H). 19F NMR (CDC), 377 MHz) δ (ppm) −114.5-−107.3 (m, 2F)

### Example 3:1-(4-((7aS,8R)-4-(Difluoromethyl)-2-(4-(4-methyl-1-(3-methyl-3-oxetanyl)-1H-pyrazol-5-yl)-1-piperidinyl)-7,7a,8,9-tetrahydroazeto[1,2-a]pyrido[3,4-f]azepin-8-yl)-1-piperazinyl)-2-propen-1-one

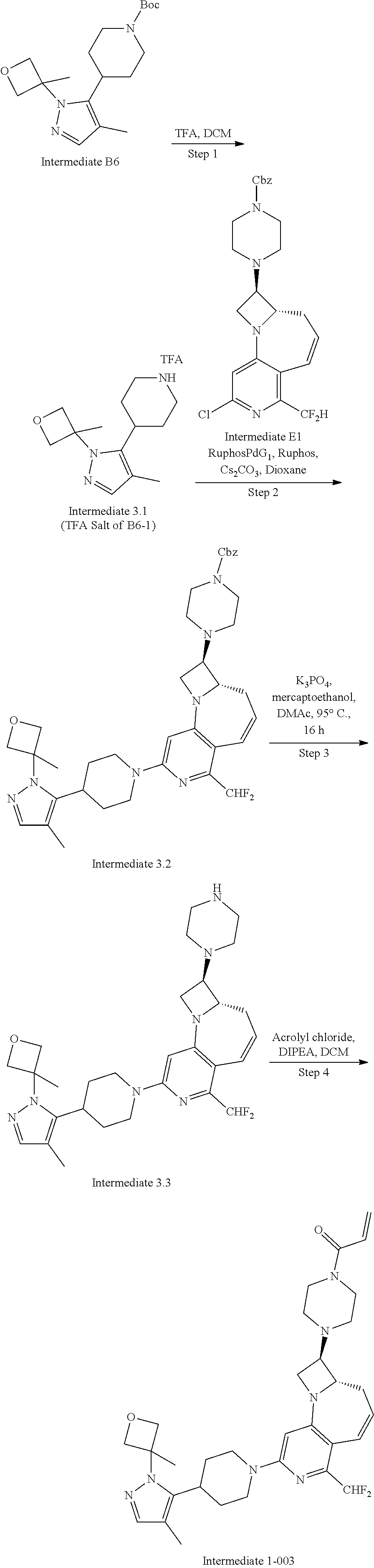

Step 1. 4-(4-Methyl-1-(3-methyloxetan-3-yl)-1H-pyrazol-5-yl)piperidine trifluoroacetate. Intermediate 3.1. To a stirred solution of Intermediate B6 (50 g, 149.2 mmol) in DCM (500 mL) was added TFA (172 mL, 2236 mmol) dropwise at 0° C. under N₂ atmosphere. The reaction mixture was stirred at rt for 2 b and concentrated to give Intermediate 3.1 (49 g) as a TFA salt, m/z (ESI): 236.1 (M+H)⁺.

Step 2. Benzyl 4-(7aS,8R)-4-(difluoromethyl)-2-(4-(4-methyl-1-(3-methyloxetan-3-yl)-1H-pyrazol-5-yl)piperidin-1-yl)-7,7a,8,9-tetrahydroazeto[1,2-a[pyrido]3,4-f]azepin-8-yl)piperazine-1-carboxylate. Intermediate 3.2. To a stirred solution of Intermediate 3.1 (42.0 g. 126 mmol) in 1,4-dioxane (1.50 L) at 0° C. was added Cs₂CO₃(686 g, 2.11 mol) and stirred for 10 min. A solution of Intermediate E1 (50.0 g, 105 mmol) in 1,4-dioxane (75 mL) was added dropwise and purged with N₂ for 10 min. RuPhos (4.91 g, 10.53 mmol) and RuPhos Pd G1 (8.60 g, 10.53 mmol) were added to the reaction mixture and stirred for 2 h at 90° C. The reaction mixture was quenched with water (1000 mL) and extracted with EtOAc (2×2000 mL). The combined organic extracts were dried over Na₂SO₄, filtered, and concentrated. The crude material was purified by chromatography, eluting with a gradient of 30-60% EtOAc in hexanes to afford Intermediate 3.2 (39.0 g, 40% yield), m/z (ESI): 674.3 (M+H)⁺. ¹H. NMR (400 MHz, DMSO-d₆) δ 7.43-7.26 (m, 5H), 7.18 (s, 1H), 6.94-6.58 (m, 2H), 5.85-5.60 (m, 2H), 5.13-5.05 (m, 4H), 4.65 (d, J=6.4 Hz, 2H), 4.42 (d, J=11.2 Hz, 2H), 3.92 (t,)=7.4 Hz, 1H), 3.78 (q, J=6.0 Hz, 1H), 3.67 (t, J=7.4 Hz, 1H), 3.41 (br s, 4H), 2.98 (q, J=6.6 Hz, 1H), 2.81 (t, J=12.8 Hz, 2H), 2.65-2.55 (m, 2H), 2.33 (dq, J=11.2, 6.1 Hz, 5H), 1.98 (d, J=9.9 Hz, 3H), 1.86 (d, J=12.3 Hz, 2H), 1.73 (d, J=15.4 Hz, 5H)

Step 3. (7aS,8R)-4-(Difluoromethyl)-2-(4-(4-methyl-1-(3-methyloxetan-3-yl)-1H-pyrazol-5-yl)piperidin-1-yl)-8-(piperazin-1-yl)-7,7a,8,9-tetrahydroazeto[1,2-a]pyrido[3,4-f]azepine, Intermediate 3.3. To a stirred solution of benzyl 4-((7aS,8R)-4-(difluoromethyl)-2-(4-(4-methyl-1-(3-methyloxetan-3-yl)-1H-pyrazol-5-yl)piperidin-1-yl)-7,7a,8,9-tetrahydroazeto[1,2-a]pyrido[3,4-f]azepin-8-yl)piperazine-1-carboxylate (9 g, 13.3 mmol) in DMAc (90 mL) was added tripotassium phosphate (11.34 g, 53.4 mmol), and the reaction mixture was degassed and purged with N₂ for 10 min. 2-Mercaptoethan-1-ol (1.880 mL, 26.7 mmol) was added to the reaction mixture and stirred at 95° C. for 16 h. The reaction mixture was quenched with water (40 mL) and extracted with EtOAc (2×30 mL). The combined organic extracts were dried (Na₂SO₄), filtered, and concentrated under reduced pressure. The crude residue was purified by reverse phase chromatography through a Gold 100-G C18 column eluting with a gradient of 0% to 70% acetonitrile in water (+0.1% TFA). The desired product was obtained in clean fractions, which were collected, and TFA was neutralized by stirring the desired mixture with sat. solution of K₂CO₃ (aq.) for 30 min. The solution was extracted with EtOAc, organic layers were combined, washed with brine, dried over Na₂SO₄ and filtered. The filtrate was concentrated under reduced pressure to provide Intermediate 3.3 (6.8 g. 12.60 mmol, 94% yield), m/z (ESI): 540.3 (M+H)⁺. ¹H NMR (400 MHz, DMSO-d₆): δ ppm 7.18 (s, 1H), 6.95-6.60 (m, 2H), 5.77 (d, J=7.3 Hz, 2H), 5.71 (ddd, J=12.6, 5.3. 4.0 H), 5.08 (d, J=6.2 Hz, 2H), 4.62-4.68 (m, 2H), 4.44 (t, J=11.3 Hz, 2H), 3.90 (t, J=7.4 Hz, 1H), 3.75 (q, J=6.1 Hz, 1H), 3.64 (t, J=7.4 Hz, 1H), 2.90 (q. J=6.6 Hz, 1H), 2.68-2.87 (m, 6H), 2.59-2.66 (m, 2H), 2.39-2.15 (m, 5H), 1.97 (s, 3H), 1.77-1.90 (m, 2H), 1.73 (d, J=15.5 Hz, 5H).

Step 4. 1-(4-((7aS,8R)-4-(Difluoromethyl)-2-(4-(4-methyl-1-(3-methyl-3-oxetanyl)-1H-pyrazol-5-yl)-1-piperidinyl)-7,7a,8,9-tetrahydroazeto[1,2-a]pyrido[3,4-f]azepin-8-yl)-1-piperazinyl)-2-propen-1-one, Compound 1-003. To a solution of Intermediate 3.3 (60 mg, 0.111 mmol) and DIPEA (43 mg, 0,334 mmol, Sigma-Aldrich Corporation) in DCM (0.6 mL), was added acryloyl chloride (20 mg, 0.222 mmol, Sigma-Aldrich Corporation) dropwise under N₂. The reaction mixture was stirred at rt for 1 h. Then, the reaction mixture was concentrated and purified by reverse phase chromatography through a C-18 column, eluting with a gradient of 0% to 100% ACN in water (+0.1% formic acid). Next, a sat. solution of K₂CO₃ (aq) was added (pH=10) and extracted with a solution of 5% MeOH in EtOAc. The combined organic extracts were dried over Na₂SO₄, filtered, and concentrated to provide 1-(4-((7aS,8R)-4-(difluoromethyl)-2-(4-(4-methyl-1-(3-methyloxetan-3-yl)-1H-pyrazol-5-yl)piperidin-1-yl)-7,7a,8,9-tetrahydroazeto[1,2-a]pyrido[3,4-f]azepin-8-yl)piperazin-1-yl)prop-2-en-1-one, Compound 1-003, m/z (ESI): 594.3 (M+H)⁺. ¹H NMR (DMSO-d₆, 400 MHz): δ ppm 7.18 (s, 1H), 6.75-6.86 (m, 2H), 6.61-6.69 (m, 1H), 6.11 (dd, J=16.7, 2.4 Hz, 1H), 5.79 (s, 1H), 5.64-5.77 (m, 2H), 5.08 (d, J=6.4 Hz, 2H), 4.65 (d, J=6.2 Hz, 2H), 4.44 (t, J=10.9 Hz, 2H), 3.92 (t, J=7.4 Hz, 1H), 3.80 (q, J=6.1 Hz, 1H), 3.70 (t, J=7.4 Hz, 1H), 3.64-3.49 (m, 4H), 2.98 (q, J=6.6 Hz, 1H), 2.81 (tt, J=12.6, 3.1 Hz, 2H), 2.59-2.67 (m, 2H), 2.34 (dd, J=15.5, 7.9 Hz, 5H). 1.97 (s, 3H), 1.78-1.92 (m, 2H), 1.74-1.78 (m, 2H), 1.71 (s, 3H).

Compounds in Table 2-1 were prepared following the procedure described for Method 1, using appropriate starting materials. All starting materials are commercially available or are described in the Intermediates section above.

TABLE 2-1

| Compound | | LCMS : (ESI + ve ion) | |
|---|---|---|---|
| # | Chemical Structure & Name | m/z ; NMR | Comments |
| 1-004 | 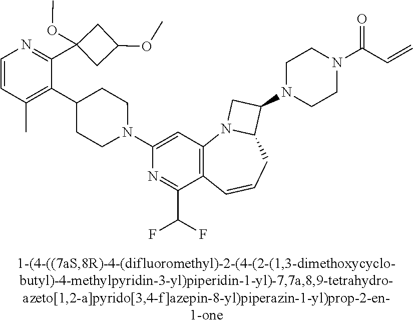 [SMILES: C=CC(=O)N1CCN([C@@H]2CN3c4cc(N5CCC(c6c(C)ccnc6C6(OC)CC(OC)C6)CC5)nc(C(F)F)c4C=CC[C@@H]23)CC1] | m/z (ESI)= 649.4 (M + H)⁺. ¹H NMR (CDCl₃, 400 MHz) δ (ppm) ppm 8.25 (d, J = 4.8 Hz, 1 H), 6.99 (d, J = 5.0 Hz, 1 H), 6.85 (br d, J = 12.3 Hz, 1 H), 6.39-6.71 (m, 2 H), 6.31 (dd, J = 16.7, 1.9 Hz, 1 H), 5.73 (br dd, J = 10.6, 1.8 Hz, 2 H), 5.62 (s, 1 H), 4.45 (br dd, J = 36.3, 12.6 Hz, 2 H), 3.92- 4.02 (m, 2 H), 3.75-3.85 (m, 1 H), 3.58 (s, 6 H), 3.30 (s, 3 H), 3.20-3.29 (m, 2 H), 2.97-3.04 (m, 1 H), 2.95 (s, 3 H), 2.83-2.92 (m, 2 H), 2.69-2.80 (m, 1 H), 2.57- 2.67 (m, 1 H), 2.42-2.49 (m, 1 H), 2.41-2.48 (m, 5 H), 2.30-2.40 (m, 3 H), 2.13- | Intermediate B3 was used |
| | | 2.28 (m, 2 H), 1.68 (br d, | |
| | | J = 11.9 Hz, 2 H); ¹⁹F NMR | |
| | | (CDCl₃, 376 MHz) δ (ppm) | |
| | | −110.73 (br d, 1F, J = 59.8 | |
| | | Hz). | |
| 1-005 | 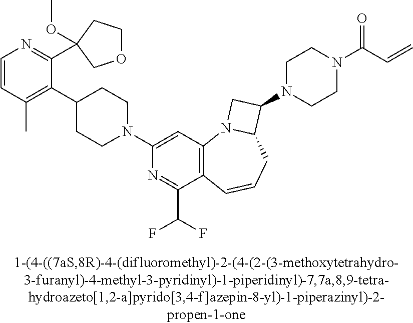 [SMILES: C=CC(=O)N1CCN([C@@H]2CN3c4cc(N5CCC(c6c(C)ccnc6C6(OC)CCOC6)CC5)nc(C(F)F)c4C=CC[C@@H]23)CC1] | m/z (ESI): 635.8 (M + H)⁺. ¹H NMR (CDCl, 500 MHz) δ (ppm) 8.23-8.25 (m, 1 H), 7.00 (d, J = 4.9 Hz, 1 H), 6.85 (br d, J = 12.6 Hz, 1 H), 6.43-6.68 (m, 2 H), 6.31 (dd, J = 16.7, 1.8 Hz, 1 H), 5.67-5.77 (m, 2 H), 5.62 (s, 1 H), 4.42-4.51 (m, 2 H), 4.38 (d, J = 9.6 Hz, 1 H), 4.23 (d, J = 9.7 Hz, 1 H), 4.06-4.12 (m, 1 H), 3.95- 4.01 (m, 3 H), 3.83-3.89 (m, 1 H), 3.53-3.71 (m, 4 H), 3.09 (s, 3 H), 2.97-3.04 (m, 2 H), 2.89-2.96 (m, 2 H), 2.70-2.78 (m, 2 H), 2.60-2.66 (m, 1 H), 2.49- 2.55 (m, 1 H), 2.35-2.49 (m, 7 H), 2.19-2.32 (m, 2 | Intermediate B35′ was used |
| | | H), 1.62-1.76 (m, 2 H); ¹⁹F | |
| | | NMR (471 MHz, CDCl₃) | |
| | | δ (ppm) −114.58-−107.79 | |
| | | (m, 2 F). | |
| 1-006 | 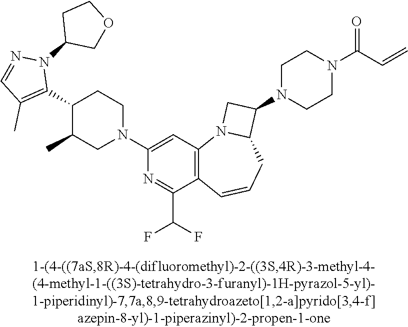 [SMILES: C=CC(=O)N1CCN([C@@H]2CN3c4cc(N5CC[C@@H](c6c(C)cnn6[C@H]6CCOC6)[C@H](C)C5)nc(C(F)F)c4C=CC[C@@H]23)CC1] | m/z (ESI): 608.2 (M + H)⁺. ¹H NMR (400 MHz, DMSO-d₆) δ (ppm) 7.20 (s, 1H), 6.92-6.63 (m, 3H), 6.10 (dd, J = 14.4, 2.4 Hz, 1H), 5.80 (s, 1H), 5.74- 5.66 (m, 2H), 5.12 (s, 1H), 4.46-4.43 (m, 2H), 4.06- 3.91 (m, 3H), 3.85-3.74 (m, 2H), 3.73-3.71 (m, 2H), 3.68-3.55 (m, 4H), 3.10- 2.98 (m, 1H), 2.84-2.70 (m, 2H), 2.64-2.62 (m, 2H), 2.49-2.26 (m, 7H), 2.11- 2.01 (m, 1H), 1.95 (s, 3H), 1.80-1.72 (m, 2H), 0.71 (d, J = 6.8, 3H) | Intermediate B40 was used |
| 1-007 | 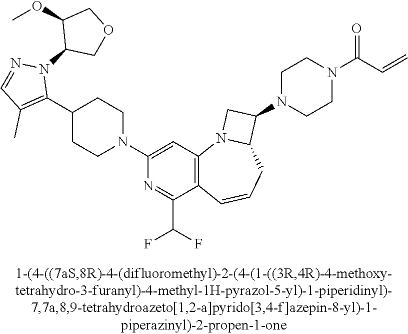 [SMILES: C=CC(=O)N1CCN([C@@H]2CN3c4cc(N5CCC(c6c(C)cnn6[C@@H]6COC[C@@H]6OC)CC5)nc(C(F)F)c4C=CC[C@@H]23)CC1] | m/z (ESI): 624.3 (M + H)⁺. ¹H NMR (DMSO-d₆, 400 MHz): δ (ppm) 7.13 (s, 1H), 6.96-6.59 (m, 3H), 6.11 (dd, J = 16.7, 2.4 Hz, 1H), 5.80 (s, 1H), 5.78-5.63 (m, 2H), 5.03 (td, J = 7.2, 5.5 Hz, 1H), 4.58-4.36 (m, 3H), 4.16-3.96 (m, 3H), 3.92 (t, J = 7.4 Hz, 1H), 3.80 (dq, J = 8.8, 5.0, 3.9 Hz, 2H), 3.70 (t, J = 7.4 Hz, 1H), 3.62-3.50 (m, 4H), 3.17 (s, 1H), 3.03- 2.94 (m, 4H), 2.92-2.81 (m, 2H), 2.75-2.60 (m, 2H), 2.36 (s, 4H), 1.99 (s, 3H), 1.96-1.65 (m, 4H). | B27/28 intermediate mixture was used |
| 1-008 | 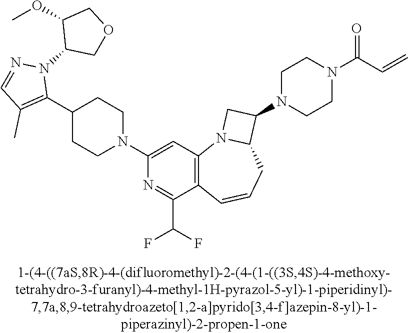 [SMILES: C=CC(=O)N1CCN([C@@H]2CN3c4cc(N5CCC(c6c(C)cnn6[C@H]6COC[C@H]6OC)CC5)nc(C(F)F)c4C=CC[C@@H]23)CC1] | m/z (ESI): 624.3 (M + H)⁺. ¹H NMR (400 MHz, DMSO-d₆) δ (ppm) 7.14 (s, 1H), 6.86-6.61 (m, 3H), 6.11 (dd, J = 16.7, 2.4 Hz, 1H), 5.80 (s, 1H), 5.77-5.64 (m, 2H), 5.03 (td, J = 7.2, 5.4 Hz, 1H), 4.47 (d, J = 12.8 Hz, 2H), 4.41 (dd, J = 8.5, 6.9 Hz, 1H), 4.14-3.98 (m, 3H), 3.93 (t, J = 7.4 Hz, 1H), 3.80 (dq, J = 8.8, 5.0, 3.9 Hz, 2H), 3.70 (t, J = 7.4 Hz, 1H), 3.55 (d, J = 12.4 Hz, 4H), 3.17 (s, 1H), 3.04-2.94 (m, 1H), 2.98 (s, 3H), 2.91-2.79 (m, 2H), 2.71-2.60 (m, 2H), 2.41-2.26 (m, 4H), 1.99(s, 3H), 1.95-1.69 (m, 4H). | B27/28 intermediate mixture was used |
| 1-009 | 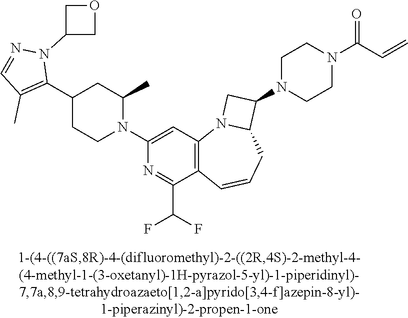 [SMILES: C=CC(=O)N1CCN([C@@H]2CN3c4cc(N5CCC(c6c(C)cnn6C6COC6)C[C@H]5C)nc(C(F)F)c4C=CC[C@@H]23)CC1] | m/z (ESI): 594.2 (M + H)⁺. ¹H NMR (400 MHz, DMSO-d₆) δ (ppm) 7.30 (s, 1H), 6.98-6.57 (m, 3H), 6.11 (dd, J = 16.7, 2.4 Hz, 1H), 5.81-5.61 (m, 4H), 4.98 (t, J = 6.2 Hz, 1H), 4.95-4.84 (m, 3H), 4.74 (br s, 1H), 4.32 (d, J = 12.9 Hz, 1H), 3.92 (t, J = 7.3 Hz, 1H), 3.79 (q, J = 6.1 Hz, 1H), 3.67 (t, J = 7.4 Hz, 1H), 3.55 (d, J = 11.1 Hz, 4H), 3.19 (t, J = 12.5 Hz, 1H), 3.05-2.85 (m, 2H), 2.71-2.60 (m, 2H), 2.35 (br s, 4H), 2.07-1.94 (m, 4H), 1.83-1.68 (m, 2H), 1.58 (d, J = 13.0 Hz, 1H), 1.21 (d, J = 6.8 Hz, 3H) | Intermediate B17 was used |
| 1-010 | 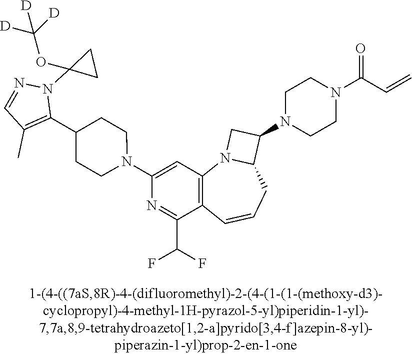 [SMILES: [2H]C([2H])([2H])OC1(n2ncc(C)c2C2CCN(c3cc4c(c(C(F)F)n3)C=CC[C@H]3[C@H](N5CCN(C(=O)C=C)CC5)CN43)CC2)CC1] | m/z (ESI): 597.0 (M + H)⁺. ¹H NMR (CDCl₃, 400 MHz) δ (ppm) 7.20 (s, 1 H), 6.85 (br d, J = 12.3 Hz, 1 H), 6.40-6.71 (m, 2 H), 6.30 (dd, J = 16.9, 1.9 Hz, 1 H), 5.69-5.77 (m, 2 H), 5.61 (s, 1 H), 4.46 (br dd, J = 26.3, 13.0 Hz, 2 H), 3.92-4.01 (m, 2 H), 3.50- 3.82 (m, 5 H), 3.43 (tt, J = 12.4, 3.6 Hz, 1 H), 2.99 (q, J = 6.5 Hz, 1 H), 2.86 (tdd, J = 12.6, 12.6, 6.4, 2.3 Hz, 2 H), 2.69-2.78 (m, 1 H), 2.57-2.69 (m, 1 H), 2.33-2.51 (m, 4 H), 2.07 (s, 3 H), 1.94-2.05 (m, 2 H), 1.83 (br d, J = 12.5 Hz, 2 H), 1.36 (br d, J = 3.8 Hz, 4 H); ¹⁹F NMR (CDCl₃, 376 MHz) δ | Intermediate B7 was used; |
| | | (ppm) −110.76 (d, 2F, | |
| | | J = 55.5 Hz). | |
| 1-011 | 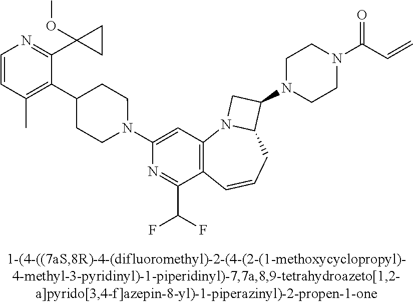 [SMILES: C=CC(=O)N1CCN([C@@H]2CN3c4cc(N5CCC(c6c(C)ccnc6C6(OC)CC6)CC5)nc(C(F)F)c4C=CC[C@@H]23)CC1] | m/z (ESI): 605.0 (M + H)⁺. ¹H NMR (CDCl₃, 400 MHz) δ (ppm) 8.24 (d, J = 4.8 Hz, 1 H), 6.98 (d, J = 5.0 Hz, 1 H), 6.85 (br d, J = 12.2 Hz, 1 H), 6.41- 6.71 (m, 2 H), 6.30 (dd, J = 16.8, 1.9 Hz, 1 H), 5.69-5.77 (m, 2 H), 5.62 (s, 1 H), 4.51 (br d, J = 12.9 Hz, 1 H), 4.43 (br d, J = 12.9 Hz, 1 H), 3.91- 4.07 (m, 3 H), 3.54-3.86 (m, 5 H), 3.17 (s, 3 H), 2.88-3.03 (m, 3 H), 2.68- 2.84 (m, 1 H), 2.58-2.66 (m, 1 H), 2.36-2.51 (m, 7 H), 2.16-2.29 (m, 2 H), 1.73 (br d, J = 12.0 Hz, | Intermediate B5 was used |
| | | 2 H), 1.10-1.21 (m, 4 H); | |
| | | ¹⁹F NMR (CDCl₃, 377 | |
| | | MHz) δ (ppm) −112.4- | |
| | | −108.9 (m, 2.F) | |
| 1-012 | 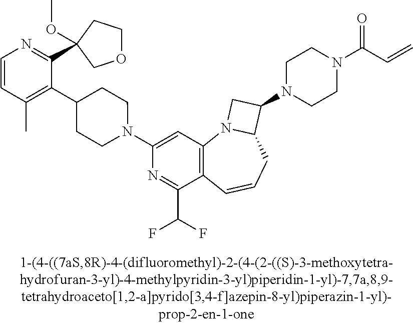 [SMILES: C=CC(=O)N1CCN([C@@H]2CN3c4cc(N5CCC(c6c(C)ccnc6[C@@]6(OC)CCOC6)CC5)nc(C(F)F)c4C=CC[C@@H]23)CC1] | m/z (ESI): 635.1(M + H)⁺. ¹H NMR (CDCl₃, 500 MHz) δ (ppm) 8.23-8.25 (m, 1 H), 7.00 (d, J = 4.9 Hz, 1 H), 6.85 (br d, J = 12.6 Hz, 1 H), 6.43-6.68 (m, 2 H), 6.31 (dd, J = 16.7, 1.8 Hz, 1 H), 5.67- 5.77 (m, 2 H), 5.62 (s, 1 H), 4.42-4.51 (m, 2 H), 4.38 (d, J = 9.6 Hz, 1 H), 4.23 (d, J = 9.7 Hz, 1 H), 4.06-4.12 (m, 1 H), 3.95- 4.01 (m, 3 H), 3.83-3.89 (m, 1 H), 3.53-3.71 (m, 4 H), 3.09 (s, 3 H), 2.97- 3.04 (m, 2 H), 2.89-2.96 (m, 2 H), 2.70-2.78 (m, 2 H), 2.60-2.66 (m, 1 H), 2.49-2.55 (m, 1 H), 2.35- | Intermediate B36 was used |
| | | 2.49 (m, 7 H), 2.19-2.32 | |
| | | (m, 2 H), 1.62-1.76 (m, 2 | |
| | | H); ¹⁹F NMR (CDCl₃, 471 | |
| | | MHz) δ (ppm) −68.80 (br | |
| | | s, 3F) (TFA), −114.7- | |
| | | −106.7 (m, 2F) | |
| 1-013 | 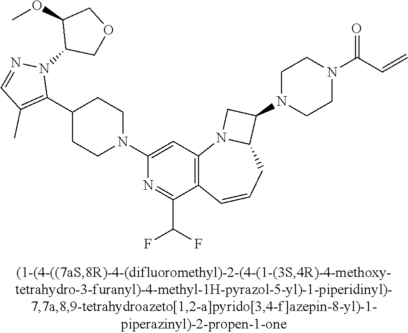 [SMILES: C=CC(=O)N1CCN([C@@H]2CN3c4cc(N5CCC(c6c(C)cnn6[C@H]6COC[C@@H]6OC)CC5)nc(C(F)F)c4C=CC[C@@H]23)CC1] | m/z (ESI): 624.3 (M + H)⁺. ¹H NMR (400 MHz, DMSO-d₆) δ (ppm) 7.21 (s, 1H), 6.94-6.61 (m, 3H), 6.11 (dd, J = 16.7, 2.4 Hz, 1H), 5.80 (s, 1H), 5.76- 5.65 (m, 2H), 5.01-4.91 (m, 1H), 4.47 (t, J = 11.7 Hz, 2H), 4.26-4.16 (m, 2H), 4.00 (dd, J = 9.7, 4.9 Hz, 1H), 3.92 (t, J = 7.4 Hz, 1H), 3.88-3.78 (m, 2H), 3.77-3.67 (m, 2H), 3.55 (d, J = 12.3 Hz, 4H), 3.27 (s, 3H), 3.24-3.14 (m, 1H), 2.99 (q, J = 6.6 Hz, 1H), 2.87 (q, J = 10.0, 9.1 Hz, 2H), 2.69-2.58 (m, 2H), 2.36 (br s, 4H), 2.00 (s, 3H), 1.92-1.68 (m, 4H). | B29/B30 intermediate trans mixtures was used |
| 1-014 | 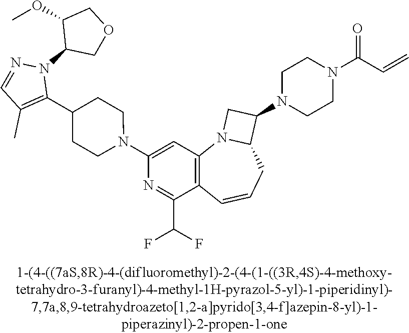 [SMILES: C=CC(=O)N1CCN([C@@H]2CN3c4cc(N5CCC(c6c(C)cnn6[C@@H]6COC[C@H]6OC)CC5)nc(C(F)F)c4C=CC[C@@H]23)CC1] | m/z (BSI): 624.4 (M + H)⁺. ¹H NMR (400 MHz, DMSO-d₆) δ (ppm) 7.21 (s, 1H), 6.94-6.62 (m, 3H), 6.11 (dd, J = 16.7. 2.4 Hz, 1H), 5.80 (s, 1H), 5.76- 5.66 (m, 2H), 4.95 (d, J = 5.5 Hz, 1H), 4.47 (t, J = 11.1 Hz, 2H), 4.25-4.17 (m, 2H), 4.00 (dd, J = 9.7, 4.9 Hz, 1H), 3.92 (t, J = 7.4 Hz, 1H), 3.87-3.68 (m, 4H), 3.55 (d, J = 12.8 Hz, 4H), 3.28 (s, 3H), 3.19 (br s, 1H), 2.99 (q, J = 6.6 Hz, 1H), 2.87 (t, J = 11.9 Hz, 2H), 2.68-2.62 (m, 2H), 2.36 (br s, 4H), 2.00 (s, 3H), 1.90-1.72 (m, 4H). | Intermediate B30 was used |
| 1-015 | 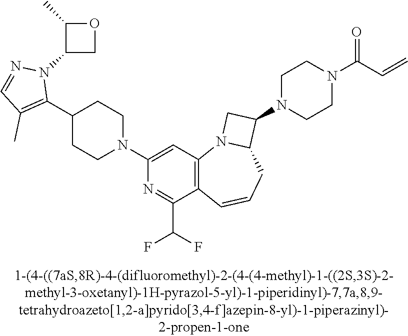 [SMILES: C=CC(=O)N1CCN([C@@H]2CN3c4cc(N5CCC(c6c(C)cnn6[C@H]6CO[C@H]6C)CC5)nc(C(F)F)c4C=CC[C@@H]23)CC1] | m/z (ESI): 593.9 (M + H)⁺. ¹H NMR (CDCl₃, 500 MHz) δ (ppm) 7.35 (s, 1 H), 6.86 (br d, J = 12.7 Hz, 1 H), 6.45-6.69 (m, 2 H), 6.32 (dd, J = 16.8, 1.9 Hz, 1 H), 5.75-5.80 (m, 1 H), 5.73 (dd, J = 10.6, 1.9 Hz, 1 H), 5.62 (s, 1 H), 5.41 (quin, J = 6.2 Hz, 1 H), 5.06-5.10 (m, 1 H), 4.95 (q, J = 7.1 Hz, 1 H), 4.77 (dd, J = 7.5, 6.2 Hz, 1 H), 4.44-4.53 (m, 2 H), 3.96- 4.01 (m, 2 H), 3.76-3.84 (m, 1 H), 3.54-3.73 (m, 4 H), 3.01 (q, J = 6.7 Hz, 1 H), 2.72-2.90 (m, 4 H), 2.61-2.68 (m, 1 H), 2.36- 2.51 (m, 4 H), 2.09 (s, 3 H), 1.97-2.05 (m, 2 H), | Intermediate B31 was used |
| | | 1.73-1.82 (m, 2 H), 1.54 | |
| | | (d, J = 6.2 Hz, 3 H); ¹⁹F | |
| | | NMR (CDCl₃, 471 MHz) | |
| | | δ (ppm) −-112.3-−109.5 | |
| | | (m, 2F). | |
| 1-016 | 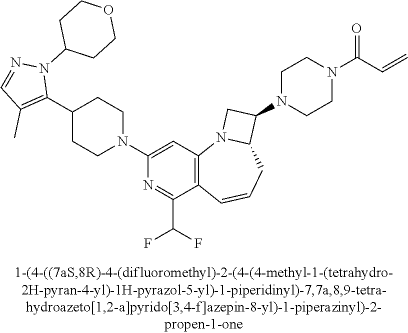 [SMILES: C=CC(=O)N1CCN([C@@H]2CN3c4cc(N5CCC(c6c(C)cnn6C6CCOCC6)CC5)nc(C(F)F)c4C=CC[C@@H]23)CC1] | m/z (ESI): 608.3 (M + H)⁺. ¹H NMR (400 MHz, DMSO-d₆) δ (ppm) 7.16 (s, 1H), 6.95-6.60 (m, 3H), 6.11 (dd, J = 16.7, 2.4 Hz, 1H), 5.80 (s, 1H), 5.76- 5.66 (m, 2H), 4.52-4.38 (m, 3H), 3.99-3.89 (m, 3H), 3.81 (q, J = 6.1 Hz, 1H), 3.70 (t, J = 7.4 Hz, 1H), 3.55 (d, J = 12.1 Hz, 4H), 3.46 (tt, J = 11.9. 2.3 Hz, 2H), 3.20-3.10 (m, 1H), 2.99 (q, J = 6.6 Hz, 1H), 2.89 (t, J = 12.4 Hz, 2H), 2.64 (t, J = 5.6 Hz, 2H), 2.34 (d, J = 13.8 Hz, 4H), 2.06 (td, J = 12.2, 4.5 Hz, 2H), 1.98 (s, 3H), 1.84 (t, J = 12.4 Hz, 2H), 1.72 (t, J = 11.1 Hz, 4H). | Intermediate B32 were used |
| 1-045 | 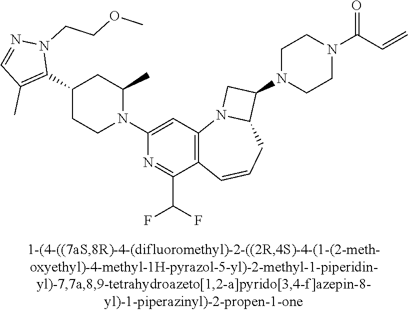 [SMILES: C=CC(=O)N1CCN([C@@H]2CN3c4cc(N5CC[C@H](c6c(C)cnn6CCOC)C[C@H]5C)nc(C(F)F)c4C=CC[C@@H]23)CC1] | m/z (ESI): 596.3 (M + H)⁺. ¹H NMR (400 MHz, DMSO-d₆) δ (ppm) 7.13 (s, 1H), 6.95-6.60 (m, 3H), 6.11 (dd, J ]= 16.7, 2.4 Hz, 1H), 5.76-5.63 (m, 3H), 4.76 (s, 1H), 4.30 (m, 2H), 4.19 (dt, J = 14.6, 5.1 Hz, 1H), 3.92 (t, J = 7.3 Hz, 1H), 3.79 (q, J = 6.1 Hz, 1H), 3.71-3.43 (m, 7H), 3.23 (s, 4H), 2.98 (m, 2H), 2.72-2.59 (m, 1H), 2.36 (m, 4H), 2.02 (m, 4H), 1.81 (m, 2H), 1.66 (d, J = 12.8 Hz, 1H), 1.19 (d, J = 6.8 Hz, 3H). One proton overlapped with DMSO signal. | Intermediate B18 was used |
| 1-046 | 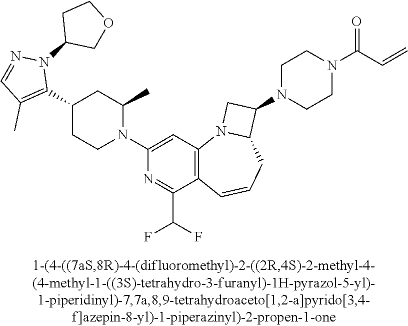 [SMILES: C=CC(=O)N1CCN([C@@H]2CN3c4cc(N5CC[C@H](c6c(C)cnn6[C@H]6CCOC6)C[C@H]5C)nc(C(F)F)c4C=CC[C@@H]23)CC1] | m/z (ESI): 608.3 (M + H)⁺. ¹H NMR (400 MHz, DMSO-d₆) δ (ppm) 7.17 (s, 1H), 6.97-6.53 (m, 3H), 6.11 (dd, J = 16.7, 2.4 Hz, 1H), 5.77-5.60 (m, 3H), 5.12 (br s, 1H), 4.76 (br s, 1H), 4.32 (br s, 1H), 4.07 (dd, J = 8.8, 7.1 Hz, 1H), 4.02-3.89 (m, 2H), 3.87-3.75 (m, 3H), 3.68 (t, J = 7.4 Hz, 1H), 3.65- 3.41 (m, 5H), 3.09-2.91 (m, 2H), 2.63 (d, J = 6.4 Hz, 2H), 2.42-2.28 (m, 6H), 2.25-2.16 (m, 1H), 2.11-2.02 (m, 3H), 1.88- 1.71 (m, 2H), 1.66 (d, J = 12.7 Hz, 1H), 1.23 (d, J = 6.8 Hz, 3H). | Intermediate B19 was used |
| 1-054 | 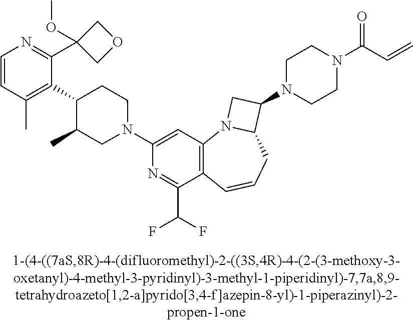 [SMILES: C=CC(=O)N1CCN([C@@H]2CN3c4cc(N5CC[C@@H](c6c(C)ccnc6C6(OC)COC6)[C@H](C)C5)nc(C(F)F)c4C=CC[C@@H]23)CC1] | m/z (ESI): 635.3 (M + H)⁺. ¹H NMR (400 MHz, DMSO-d₆) δ (ppm) 8.29 (d, J = 4.9 Hz, 1H), 7.18 (d, J = 5.0 Hz, 1H), 6.97- 6.59 (m, 3H), 6.11 (dd, J = 16.7, 2.4 Hz, 1H), 5.80 (s, 1H), 5.76-5.61 (m, 2H), 5.14 (dd, J] = 11.6, 7.2 Hz, 2H), 4.78 (t, J = 7.2 Hz, 2H), 4.43 (t, J = 13.1 Hz, 2H), 3.93 (t, J = 7.4 Hz, 1H), 3.80 (q, J = 6.1 Hz, 1H), 3.71 (t, J = 7.4 Hz, 1H), 3.55 (d, J = 11.9 Hz, 4H), 3.05-2.91 (m, 4H), 2.76 (t, J = 12.0 Hz, 1H), 2.64 (t, J = 5.2 Hz, 2H), 2.37 (d, J = 8.3 Hz, 10H), 2.03-1.87 (m, 1H), 1.66 | Intermediate B39 was used |
| | | (d, J = 12.8 Hz, 1H), 0.67 | |
| | | (d, J = 5.9 Hz, 3H). | |
| 1-060 | 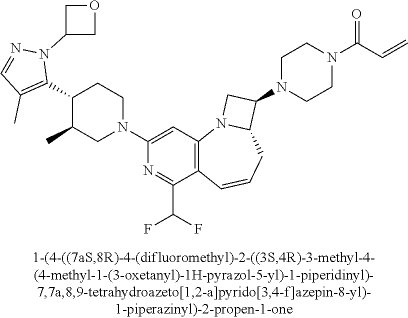 [SMILES: C=CC(=O)N1CCN([C@@H]2CN3c4cc(N5CC[C@@H](c6c(C)cnn6C6COC6)[C@H](C)C5)nc(C(F)F)c4C=CC[C@@H]23)CC1] | m/z (ESI): 594.3 (M + H)⁺. ¹H NMR (500 MHz, MeOH-d4) δ (ppm) 7.37 (s, 1 H), 6.76 (dd, J = 16.8, 10.7 Hz, 2 H), 6.43- 6.69 (m, 1 H), 6.21 (dd, J = 16.8, 1.9 Hz, 1 H), 5.75-5.78 (m, 2 H), 5.72- 5.75 (m, 1 H), 5.11 (br t, J = 5.8 Hz, 1 H), 5.05 (t, J = 6.3 Hz, 1 H), 4.98 (q, J = 6.6 Hz, 2 H), 4.42-4.49 (m, 2 H), 4.00 (t, J = 7.3 Hz, 1 H), 3.91 (q, J = 6.3 Hz, 1 H), 3.64-3.75 (m, 5 H), 3.35-3.40 (m, 1 H), 3.03 (q, J = 6.7 Hz, 1 H), 2.90 (td, J = 12.8, 2.6 Hz, 1 H), 2.68-2.72 (m, 2 H), 2.60-2.68 (m, 1 H), 2.45- | Intermediate B24 was used |
| | | 2.57 (m, 5 H), 2.06-2.14 | |
| | | (m, 1 H), 2.03 (s, 3 H), | |
| | | 1.89-1.99 (m, 1 H), 1.68 | |
| | | (br dd, J = 13.4, 3.4 Hz, | |
| | | 1 H), 0.72 (d, J = 6.6 Hz, | |
| | | 3 H) | |
| 1-063 | 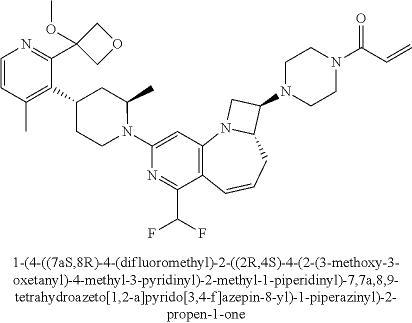 [SMILES: C=CC(=O)N1CCN([C@@H]2CN3c4cc(N5CC[C@H](c6c(C)ccnc6C6(OC)COC6)C[C@H]5C)nc(C(F)F)c4C=CC[C@@H]23)CC1] | m/z (ESI): 635.3 (M + H)⁺. ¹H NMR (400 MHz, DMSO-d₆) δ (ppm) 8.30 (d, J = 4.9 Hz, 1H), 7.19 (d, J = 5.0 Hz, 1H), 6.58- 6.97 (m, 3H), 6.11 (dd, J = 16.7, 2.4 Hz, 1H), 5.63- 5.82 (m, 3H), 5.18 (dd, J = 14.3, 7.0 Hz, 2H), 4.82 (dd, J = 14.2, 7.0 Hz, 3H), 4.34 (d, J = 13.1 Hz, 1H), 3.92 (t, J = 7.4 Hz, 1H), 3.80 (q, J = 6.0 Hz, 1H), 3.68 (t, J = 7.4 Hz, 1H), 3.55 (br s, 4H), 2.79-3.12 (m, 6H), 2.60-2.72 (m, 2H), 2.44 (s, 3H), 2.23- 2.43 (m, 5H), 2.07 (dd, J = 13.2, 4.8 Hz, 1H), 1.63 (d, J = 12.6 Hz, 1H), | Intermediate B10 was used |
| | | 1.52 (d, J = 12.8 Hz, 1H), | |
| | | 1.17 (d, J = 6.8 Hz, 3H). | |
| 1-072 | 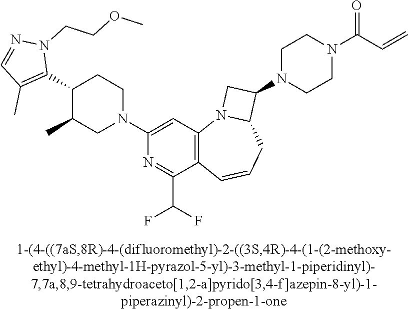 [SMILES: C=CC(=O)N1CCN([C@@H]2CN3c4cc(N5CC[C@@H](c6c(C)cnn6CCOC)[C@H](C)C5)nc(C(F)F)c4C=CC[C@@H]23)CC1] | m/z (ESI): 596.1 (M + H)⁺. ¹H NMR (500 MHz, MeOH-d₄) δ (ppm) 7.22 (s, 1 H), 6.76 (dd, J = 16.8, 10.7 Hz, 2 H), 6.43- 6.68 (m, 1 H), 6.21 (dd, J = 16.8. 1.9 Hz, 1 H), 5.69-5.78 (m, 3 H), 4.41- 4.51 (m, 2 H), 4.29-4.36 (m, 1 H), 4.18-4.26 (m, 1 H), 4.00 (t, J = 7.3 Hz, 1 H), 3.91 (q, J = 6.4 Hz, 1 H), 3.65-3.75 (m, 7 H), 3.29 (s, 3 H), 3.03 (d, J = 6.2 Hz, 1 H), 2.84-2.92 (m, 1 H), 2.76 (br d, J = 3.5 Hz, 1 H), 2.66-2.71 (m, 2 H), 2.43-2.58 (m, 5 H), 2.10-2.24 (m, 1 H), | Intermediate E1 and B41; Step 3 was omitted |
| | | 2.02 (s, 3 H), 1.86-1.97 | |
| | | (m, 1 H), 1.74-1.83 (m, 1 | |
| | | H), 0.80 (d, J = 6.6 Hz, | |
| | | 3 H). | |
| 1-083 | 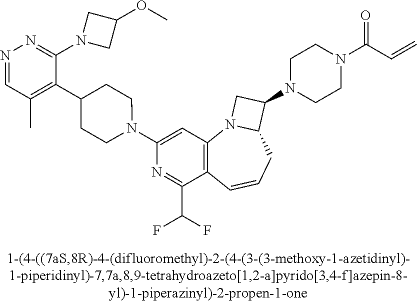 [SMILES: C=CC(=O)N1CCN([C@@H]2CN3c4cc(N5CCC(c6c(C)cnnc6N6CC(OC)C6)CC5)nc(C(F)F)c4C=CC[C@@H]23)CC1] | m/z (ESI): 621.0 (M + H)⁺ ¹H NMR (400 MHz, CDCl₃) δ (ppm) 8.42 (s, 1 H), 6.86 (br d, J = 12.5 Hz, 1 H), 6.40-6.74 (m, 2 H), 6.31 (dd, J = 16.7, 1.9 Hz, 1 H), 5.77 (td, J = 6.4, 2.9 Hz, 1 H), 5.74 (br d, J = 1.9 Hz, 1 H), 5.62 (s, 1 H), 4.50 (br dd, J = 30.6, 13.1 Hz, 2 H), 4.30-4.41 (m, 3 H), 4.09-4.15 (m, 2 H), 3.93-4.02 (m, 2 H), 3.51-3.84 (m, 5 H), 3.38 (s, 3 H), 2.94-3.04 (m, 2 H), 2.80-2.91 (m, 2 H), 2.69-2.79 (m, 1 H), 2.58- 2.69 (m, 1 H), 2.37-2.52 (m, 4 H), 2.30-2.36 (m, 3 | Intermediate B46 was used |
| | | H), 2.18 (qdd, J = 12.5, | |
| | | 12.5, 12.5, 8.3, 4.3 Hz, | |
| | | 2 H), 1.72 (br d, J = 12.3 | |
| | | Hz, 2 H) ¹⁹F NMR (376 | |
| | | MHz, CDCl₃) δ (ppm) | |
| | | −112.26-−109.82 (m, 2 F); | |

### Method 2

### Example 17:1-(4-((7aS,8R)-4-(Difluoromethyl)-2-(4-(2-(3-methoxy-3-oxetanyl)-4-methyl-3-pyridinyl)-1-piperidinyl)-5,6,7,7a, 8,9-hexahydroazeto[1,2-a]pyrido[3,4-f]azepin-8-yl)-1-piperazinyl)-2-propen-1-one

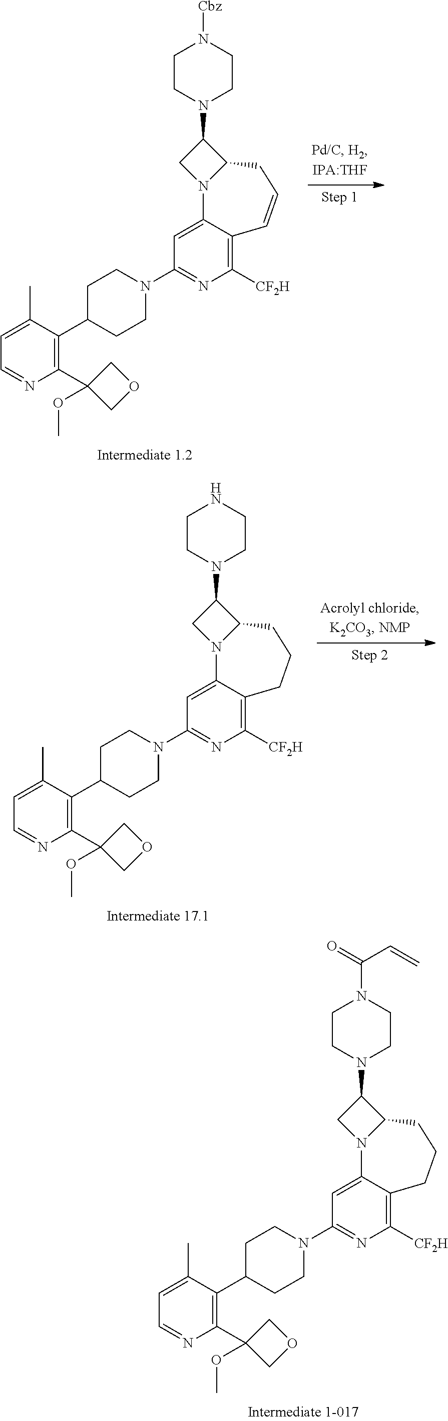 [SMILES: COC1(c2nccc(C)c2C2CCN(c3cc4c(c(C(F)F)n3)C=CC[C@H]3[C@H](N5CCN(C(=O)OCc6ccccc6)CC5)CN43)CC2)COC1 ; COC1(c2nccc(C)c2C2CCN(c3cc4c(c(C(F)F)n3)CCC[C@H]3[C@H](N5CCNCC5)CN43)CC2)COC1 ; C=CC(=O)N1CCN([C@@H]2CN3c4cc(N5CCC(c6c(C)ccnc6C6(OC)COC6)CC5)nc(C(F)F)c4CCC[C@@H]23)CC1]

Step 1. (7aS,8R)-4-(Difluoromethyl)-2-(4-(2-(3-methoxyoxetan-3-yl)-4-methylpyridin-3-yl)piperidin-1-yl)-8-(piperazin-1-yl)-5,6,7,7a,8,9-hexahydroazeto[1,2-a]pyrido[3,4-f]azepine, Intermediate 17.1. To a stirred solution of Intermediate 1.2 (60 g, 86 mmol) in IPA (420 mL) and THF (840 mL), palladium on activated carbon (10 wt % dry, 18.22 g, 17.12 mmol) was added, and the solution was degassed thoroughly and stirred under H₂ atmosphere (14 psi) for 96 h at rt. The reaction mixture was filtered, washed with 10% MeOH in DCM (1000 mL), and concentrated to give Intermediate 17.1 (47 g, 83 mmol, 97% yield), m/z (ESI): 569.3 (M+H)+¹H NMR (400 MHz, DMSO-d₆): δ (ppm) 8.29 (d, J=4.9 Hz, 1H), 7.18 (d, J=5.0 Hz, 1H), 6.70 (t, J=54.8 Hz, 1H), 5.93 (s, 1H), 5.14 (d, J=7.1 Hz, 2H), 4.81 (d, J=7.1 Hz, 2H), 4.48-4.32 (m, 2H), 3.75-3.60 (m, 3H), 3.06-2.98 (m, 1H), 2.96 (s, 3H), 2.80 (q, J=6.6 Hz, 1H), 2.75-2.60 (m, 7H), 2.39 (s, 4H), 2.41-2.32 (m, 4H), 2.14-1.99 (m, 3H), 1.94-1.69 (m, 3H), 1.58 (d, J=11.9 Hz, 2H), 1.40 (s, 1H).

Step 2. (7aS,8R)-4-(Difluoromethyl)-2-(4-(2-(3-methoxyoxetan-3-yl)-4-methylpyridin-3-yl)piperidin-1-yl)-8-(piperazin-1-yl)-5,6,7,7a,8,9-hexahydroazeto[1,2-a]pyrido[3,4-f]azepine, Compound 1-017. To a stirred solution of Intermediate 17.1 (120 g, 211 mmol) in NMP (1200 mL) was added K₂CO₃ (291 g, 211 mmol) at −15° C., and stirred for 15 min. At −15° C., a solution of acryloyl chloride (22.91 g, 253 mmol) in NMP (60 mL) was added dropwise, and the reaction mixture was stirred for 15 min. The reaction mixture was slowly quenched with crushed ice water (5000 mL), stirred for 10 min, and extracted with EtOAc (2×5000 mL). The combined organic extracts were washed with water (3×5000 mL) followed by brine (5000 mL), dried with Na₂SO₄, filtered, and concentrated. The crude material was purified by flash chromatography eluting with 0 to 3% MeOH in EtOAc to give 1-(4-((7aS,8R)-4-(difluoromethyl)-2-(4-(2-(3-methoxy oxetan-3-yl)-4-methylpyridin-3-yl)piperidin-1-yl)-5,6,7,7a,8,9-hexahydroazeto[1,2-a]pyrido[3,4-f]azepin-8-yl)piperazin-1-yl)prop-2-en-1-one, Compound 1-017 (110 g, 176 mmol, 83% yield), m/z (ESI): 623.3 (M+H)⁺. ¹H NMR (400 MHz, DMSO-d₆): δ (ppm) 8.28 (d, J=4.9 Hz, 1H). 7.17 (d, J=5.0 Hz, 1H). 6.80 (dd, J=16.9, 10.7 Hz, 1H), 6.63 (d, J=54.8 Hz, 1H), 6.11 (dd, J=16.7, 2.4 Hz, 1H), 5.94 (s, 1H), 5.68 (dd, J=10.4, 2.4 Hz, 1H), 5.14 (d, J=7.1 Hz, 2H), 4.81 (d, J=7.1 Hz, 2H), 4.50-4.25 (m, 2H), 3.78-3.64 (m, 3H), 3.55 (d, J=10.7 Hz, 4H), 3.08-2.95 (m, 4H), 2.89 (q, J=6.5 Hz, 1H), 2.71 (h, J=12.6, 11.8 Hz, 3H), 2.35-2.24 (m, 8H), 2.13-1.95 (m, 2H), 1.97-1.70 (m, 3H), 1.58 (d, J=12.6 Hz, 2H), 1.40 (d, J=11.3 Hz, 1H).

### Example 18:1-(4-((7aS,8R)-4-(Difluoromethyl)-2-(4-(4-methyl-1-(3-methyloxetan-3-yl)-1H-pyrazol-5-yl)piperidin-1-yl)-5,6,7,7a,8,9-hexahydroazeto[1,2-a]pyrido[3,4-f]azepin-8-yl)piperazin-1-yl)prop-2-en-1-one

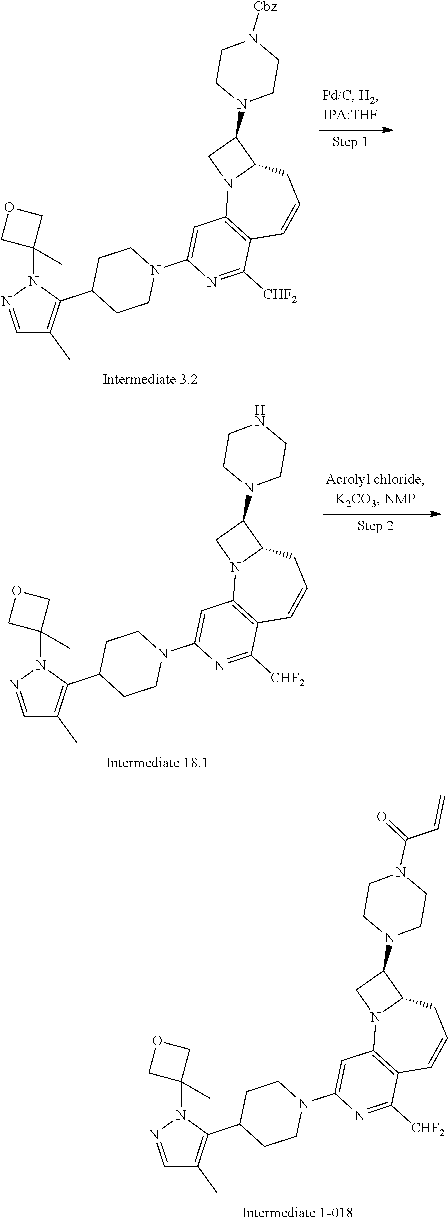 [SMILES: Cc1cnn(C2(C)COC2)c1C1CCN(c2cc3c(c(C(F)F)n2)C=CC[C@H]2[C@H](N4CCN(C(=O)OCc5ccccc5)CC4)CN32)CC1 ; Cc1cnn(C2(C)COC2)c1C1CCN(c2cc3c(c(C(F)F)n2)C=CC[C@H]2[C@H](N4CCNCC4)CN32)CC1 ; C=CC(=O)N1CCN([C@@H]2CN3c4cc(N5CCC(c6c(C)cnn6C6(C)COC6)CC5)nc(C(F)F)c4C=CC[C@@H]23)CC1]

Step 1. (7aS,8R)-4-(Difluoromethyl)-2-(4-(4-methyl-1-(3-methyloxetan-3-yl) ˜1H-pyrazol-5-yl)piperidin-1-yl)-8-(piperazin-1-yl)-5,6,7,7a,8,9-hexahydroazeto[1,2-a]pyrido[3,4-f]azepine, Intermediate 18.1. To a stirred solution of Intermediate 3.2 (24 g, 35.6 mmol) in IPA (220 mL) and THF (330 mL) was added palladium on activated carbon (10 wt % dry basis, 10.42 g). The reaction mixture was degassed thoroughly and stirred under H₂ pressure (14 psi) for 16 h at rt. The reaction mixture was filtered, washed with 10% MeOH in DCM (2.0 L) and concentrated. The crude was purified by Prep HPLC (X Bridge C₈ (100×19) mm 5.0 μm column with a mobile phase of 0.1% ammonia in water and ACN using a flow rate of 15 mL/min to afford Intermediate 18.1 (12.0 g, 63% yield), m/z (ESI): 542.3 (M+H)⁺. ¹H NMR (400 MHz, DMSO-d₆): δ (ppm) 7.18 (s, 1H), 6.70 (t, J=54 Hz, 1H), 5.92 (s, 1H), 5.07 (d, J=6.2 Hz, 2H), 4.65 (d, J=6.4 Hz, 2H), 4.39 (t, J=10.6 Hz, 2H), 3.70 (t, J=7.6 Hz, 1H), 3.70-3.60 (m, 2H), 3.01 (dd, J=14.6, 8.6 Hz, 1H), 2.84-2.64 (m, 7H), 2.39-2.15 (m, 7H), 1.98 (s, 3H), 1.93-1.72 (m, 7H), 1.71 (s, 3H), 1.45-1.31 (m, 1H).

Step 2. 1-(4-((7aS,8R)-4-(Difluoromethyl)-2-(4-(4-methyl-1-(3-methyloxetan-3-yl)-1H-pyrazol-5-yl)piperidin-1-yl)-5,6,7,7a,8,9-hexahydroazeto[1,2-a]pyrido[3,4-f]azepin-8-yl)piperazin-1-yl)prop-2-en-1-one, Compound 1-018. To a stirred solution of Intermediate 18.1 (12 g, 22.15 mmol) in NMP (120 mL) at 0° C. was added K₂CO₃ (61.2 g, 443 mmol) and stirred for 10 min. Acryloyl chloride (2.2 mL, 26.6 mmol) was added to the reaction mixture and stirred for 10 min at 0° C. The reaction mixture was quenched with ice cold water (200 mL) and extracted with EtOAc (2×500 mL). The combined organic extracts were washed with water (5×200 mL), dried with Na₂SO₄, filtered, and concentrated. The crude material was purified by reverse-phase preparative HPLC (YMC column, 0.1% NH₃ in water/ACN, flow rate: 15 mL/min) to give Compound 1-018 (3.5 g, 44% yield), m/z (ESI): 596.4 (M+H)⁺. ¹H NMR (400 MHz, DMSO-d₆): δ (ppm) 7.18 (s, 1H), 6.51-6.87 (m, 2H), 6.11 (dd, J=16.7, 2.4 H), 5.93 (s, 1H), 5.68 (dd, J=10.4, 2.4 Hz, 1H), 5.07 (d, J=6.2 Hz, 2H), 4.52-4.71 (m, 2H), 4.35-4.45 (m, 2H), 3.78-3.65 (m, 3H), 3.64-3.48 (m, 4H), 3.01 (t, J=11.7 Hz, 1H), 2.89 (q, J=6.6 Hz, 1H), 2.75 (tt, J=12.6, 2.7 Hz, 2H), 2.39-2.24 (m, 6H), 1.98 (s, 3H), 1.93-−1.79 (m, 4H), 1.78-1.66 (m, 6H), 1.40 (d, J=11.0 Hz, 1H).

### Example 19:1-(4-((7aS,8R)-4-(Difluoromethyl)-2-((2R,4S)-2-methyl-4-(4-methyl-1-(3-oxetanyl)-1H-pyrazol-5-yl)-1-piperidinyl)-5,6,7,7a,8,9-hexahydroazeto[1,2-a]pyrido[3,4-f]azepin-8-yl)-1-piperazinyl)-2-propen-1-one

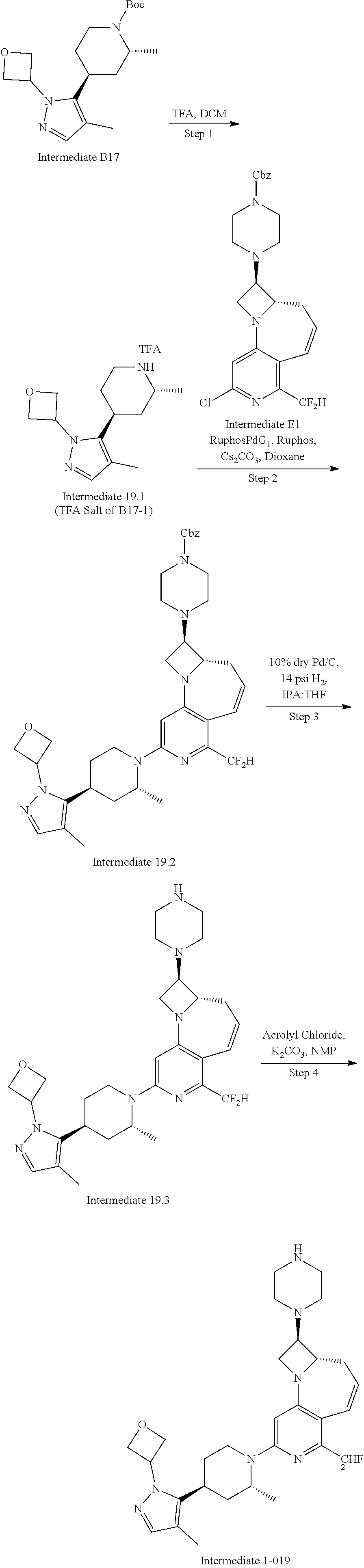

Step 1. (2R,4S)-2-methyl-4-(4-methyl-1-(oxetan-3-yl)-1H-pyrazol-5-yl)piperidine trifluoroacetate, Intermediate 19.1. To a stirred solution of Intermediate B17 (7 g, 20.87 mmol) in DCM (140 mL) at 0° C. was added TFA (23.97 mL, 313 mmol). The reaction mixture was stirred at rt for 3 h. The reaction mixture was concentrated to provide Intermediate 19.1 (quantitative) as a TFA salt, m/z (ESI): 236.2 (M+H)⁺.

Step 2. Benzyl 4-((7aS,8R)-4-(difluoromethyl)-2-((2R,4S)-2-methyl-4-(4-methyl-1-(oxetan-3-yl)-1H-pyrazol-5-yl)piperidin-1-yl)-7,7a,8,9-tetrahydroazeto[1,2-a]pyrido[3,4-f]azepin-8-yl)piperazine-1-carboxylate, Intermediate 19.2. To a stirred solution of Intermediate 19.1 (5.26 g, 15.79 mmol) in 1,4-dioxane (300 mL), was added Cs₂CO₃ (51.5 g, 158 mmol) and stirred for 5 min. Then, Intermediate E1 (7.5 g, 15.79 mmol) was added, and the reaction mixture was purged with N₂ for 2 min. Then, RuPhos Pd G1 (1.289 g, 1.579 mmol) was added followed by RuPhos (0.736 g, 1.58 mmol), and the reaction mixture was stirred for 16 h at 110° C. The reaction mixture was diluted with water (200 mL) and extracted with EtOAc (3×200 mL). The combined organic extracts were washed with brine (100 mL), dried over Na₂SO₄, filtered, and concentrated. The crude material was purified by chromatography, eluting with a gradient of 40-80% EtOAc in hexanes, to provide Intermediate 19.2 (3.7 g, 5.49 mmol, 35% yield), m/z (ESI): 674.3 (M+H)⁺.

Step 3. (7aS,8R)-4-(Difluoromethyl)-2-((2R,4S)-2-methyl-4-(4-methyl-1-(oxetan-3-yl)-1H-pyrazol-5-yl)piperidin-1-yl)-8-(piperazin-1-yl)-5,6,7,7a,8,9-hexahydroazeto[1,2-a]pyrido[3,4-f]azepine, Intermediate 19.3. To a stirred solution of Intermediate 19.2 (3.7 g, 5.49 mmol) in IPA (55.5 mL) and THF (55.5 mL) was added palladium on activated carbon (10 wt % dry basis, 2.63 g, 2.47 mmol) under N₂ atmosphere. The reaction mixture was degassed and backfilled with H₂. The reaction was stirred under H₂ pressure (10 psi) at rt for 3 h. Then, the reaction was filtered, washed with 10% MeOH in DCM, and concentrated to afford Intermediate 19.3 (2.8 g, 5.17 mmol, 94% yield), m/z (ESI): 542.1 (M+H)⁺.

Step 4. 1-(4-((7aS,8R)-4-(Difluoromethyl)-2-((2R,4S)-2-methyl-4-(4-methyl-1-(3-oxetanyl)-1H-pyrazol-5-yl)-1-piperidinyl)-5,6,7,7a,8,9-hexahydroazeto[1,2-a]pyrido[3,4-f]azepin-8-yl)-1-piperazinyl)-2-propen-1-one, Compound 1-019. To a stirred solution of Intermediate 19.3 (2.8 g, 5.17 mmol) in DCM (56.0 mL), was added DIPEA (4.51 mL, 25.8 mmol) at −78° C., and stirred for 5 min. Next, at −78° C., acryloyl chloride (0.420 mL, 5.17 mmol) in DCM (10 mL) was added drop wise and stirred for 5 min. The reaction mixture was quenched with ice water (200 mL) and extracted with DCM (200 mL×3). The combined organic extracts were washed with water, dried over Na₂SO₄ and concentrated. The crude was purified by pre-HPLC (Kinetex EVO C₁₈, NH₃ in water/ACN, flow rate 15 mL/min) to afford 1-(4-((7aS,8R)-4-(difluoromethyl)-2-((2R,4S)-2-methyl-4-(4-methyl-1-(3-oxetanyl)-1H-pyrazol-5-yl)-1-piperidinyl)-5,6,7,7a,8,9-hexahydroazeto[1,2-a]pyrido[3,4-f]azepin-8-yl)-1-piperazinyl)-2-propen-1-one, Compound 1-019 (1.6 g, 2.69 mmol, 52% yield), m/z (ESI): 596.2 (M+H)⁺. JH NMR (400 MHz, DMSO-d₆) δ (ppm) 7.30 (s, 1H), 6.88-6.52 (m, 2H), 6.11 (dd, J=16.7, 2.4 Hz, 1H), 5.86 (s, 1H), 5.76-5.64 (m, 2H), 4.98 (t, J=6.2 Hz, 1H), 4.89 (ddt, J=10.0, 6.0, 3.3 Hz, 3H), 4.72 (s, 1H), 4.28 (d, J=13.2 Hz, 1H), 3.78-3.62 (m, 3H), 3.62-3.47 (m, 4H), 3.17 (t, J=12.4 Hz, 1H), 3.08-2.84 (m, 3H), 2.34 (d, J=8.5 Hz, 5H), 2.03 (s, 3H), 2.04-1.68 (m, 6H), 1.65-1.50 (m, 1H), 1.39 (d, J=11.7 Hz, 1H), 1.18 (d, J=6.8 Hz, 3H).

### Example 20:1-(4-((7aS,8R)-4-(Difluoromethyl)-2-((2R,4S)-2-methyl-4-(4-methyl-1-((3S)-tetrahydro-3-furanyl)-1H-pyrazol-5-yl)-1-piperidinyl)-5,6,7,7a,8,9-hexahydroazeto[1,2-a]pyrido[3,4-f]azepin-8-yl)-1-piperazinyl)-2-propen-1-one

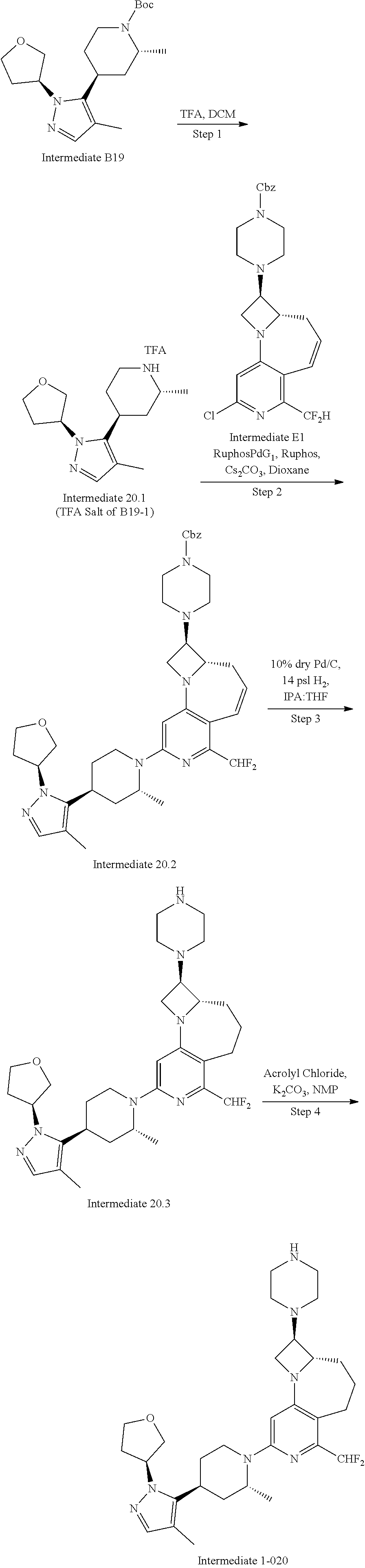

Step 1. (2R,4S)-2-Methyl-4-(4-methyl-1-((S)-tetrahydrofuran-3-yl)-1H-pyrazol-5-yl)piperidine trifluoroacetate, Intermediate 20.1. To a solution of Intermediate B19 (7.2 g, 20.60 mmol) in DCM (108 mL) at 0° C., was added TFA (23.81 mL, 309 mmol). The reaction mixture was stirred at rt for 3 h and concentrated to provide Intermediate 20.1 as a TFA salt (7 g, 20.21 mmol, 98% yield), m/z (ESI): 250.2 (M+H)⁺.

Step 2. Benzyl 4-((7aS,8R)-4-(difluoromethyl)-2-((2R,4S)-2-methyl-4-(4-methyl-1-((S)-tetrahydrofuran-3-yl)-1H-pyrazol-5-yl)piperidin-1-yl)-7,7a,8,9-tetrahydroazeto[1,2-a]pyrido[3,4-f]azepin-8-yl)piperazine-1-carboxylate, Intermediate 20.2. To a stirred solution of Intermediate 20.1 (6.56 g, 18.95 mmol) and Intermediate E1 (6 g, 12.63 mmol) in 1,4-dioxane (180 mL), was added Cs₂CO₃(41.2 g, 126 mmol) and stirred for 5 min. The reaction mixture was purged by N₂ for 5 min. Then, RuPhos (0.589 g, 1.263 mmol) followed by RuPhos Pd G1 (1.031 g, 1.263 mmol) were added. The reaction mixture was stirred for 2 h at 110° C. Then, the reaction mixture was diluted with water (300 mL) and extracted with EtOAc (3×300 mL). The combined organic extracts were washed with brine (100 mL) and dried over Na₂SO₄. The solution was filtered and concentrated. The crude material was purified by chromatography, eluting with a gradient of 40% to 80% EtOAc in pet, ether to provide Intermediate 20.2 (5.2 g, 7.56 mmol, 60% yield), m/z (ESI): 688.3 (M+H)⁺.

Step 3. (7aS,8R)-4-(Difluoromethyl)-2-((2R,4S)-2-methyl-4-(4-methyl-1-((S)-tetrahydrofuran-3-yl)-1H-pyrazol-5-yl)piperidin-1-yl)-8-(piperazin-1-yl)-5,6,7,7a,8,9-hexahydroazeto[1,2-a]pyrido[3,4-f]azopine, Intermediate 20.3. A solution of intermediate 20.2 (6 g, 8.72 mmol) in IPA (60.0 mL) and THF (60.0 mL) was purged with N₂ for 5 min. Then, palladium on activated carbon (10 wt % dry basis, 3.71 g, 3.49 mmol) was added, and the reaction mixture was degassed and stirred under H₂ (14.7 psi) for 3 b at rt. The reaction mixture was filtered, washed with 10% MeOH in DCM and concentrated to provide Intermediate 20.3 (4.9 g, 8.82 mmol, 100% yield), m/z (ESI): 556.4 (M+H)⁺.

Step 4. 1-(4-((7aS,8R)-4-(difluoromethyl)-2-((2R,4S)-2-methyl-4-(4-methyl-1-((3S)-tetrahydro-3-furanyl)-1H-pyrazol-5-yl)-1-piperidinyl)-5,6,7,7a,8,9-hexahydroazeto[1,2-a]pyrido[3,4-f]azepin-8-yl)-1-piperazinyl)-2-propen-1-one, Compound 1-020. To a solution of Intermediate 20.3 (4.9 g, 8.82 mmol) in DCM (98 mL), was added DIPEA (7.70 mL, 44.1 mmol) dropwise at −78° C. and stirred for 5 min. Next, acryloyl chloride (0.860 mL, 10.58 mmol) in 5 mL DCM was added dropwise at −78° C., and stirred for 15 min. The reaction mixture was quenched by ice water (100 mL) and extracted with DCM (2×200 mL). The combined organic extracts were dried over Na₂SO₄, filtered, and concentrated. The material was purified by RP-MPLC with 5-60% 0.01% NH;/water and ACN with a flow rate of 20 mL/min to provide 1-(4-((7aS,8R)-4-(difluoromethyl)-2-((2R,4S)-2-methyl-4-(4-methyl-1-((3S)-tetrahydro-3-furanyl)-1H-pyrazol-5-yl)-1-piperidinyl)-5,6,7,7a,8,9-hexahydroazeto[1,2-a]pyrido[3,4-f]azepin-8-yl)-1-piperazinyl)-2-propen-1-one, Compound 1-020 (2.4 g. 3.94 mmol, 45% yield), m/z (ESI): 610.4 (M+H)⁺. ¹H NMR (400 MHz, DMSO-d₆) δ (ppm) 7.17 (s, 1H), 6.87-6.51 (m, 2H), 6.11 (dd, J=16.7, 2.4 Hz, 1H), 5.86 (s, 1H), 5.68 (dd, J=10.5, 2.4 Hz, 1H), 5.12 (tt, J=8.7, 4.8 Hz, 1H), 4.74 (br s, 1H), 4.29 (d, J=13.1 Hz, 1H), 4.07 (dd, J=8.8, 7.0 Hz, 1H), 3.97 (q, J=7.4 Hz, 1H), 3.84 (ddd, J=10.3, 8.2, 5.2 Hz, 2H), 3.70 (dt, J=24.7, 7.7 Hz, 3H), 3.56 (t, J=8.4 Hz, 4H), 3.37 (s, 1H), 3.14-2.92 (m, 2H), 2.89 (q, J=6.6 Hz, 1H), 2.42-2.23 (m, 6H), 2.19 (ddt, J=12.3, 7.4, 5.1 Hz, 1H), 2.10-1.98 (m, 4H), 1.94-1.70 (m, 5H), 1.65 (d, J=12.7 Hz, 1H), 1.39 (d, J=11.5 H), 1.20 (d, J=6.7 Hz, 3H).

### Example 21:1-(4-((7aS,8R)-4-(Difluoromethyl)-2-((2R,4S)-4-(1-(2-methoxyethyl)-4-methyl-1H-pyrazol-5-yl)-2-methyl-1-piperidinyl)-5,6,7,7a,8,9-hexahydroazeto[1,2-a]pyrido[3,4-f]azepin-8-yl)-1-piperazinyl)-2-propen-1-one

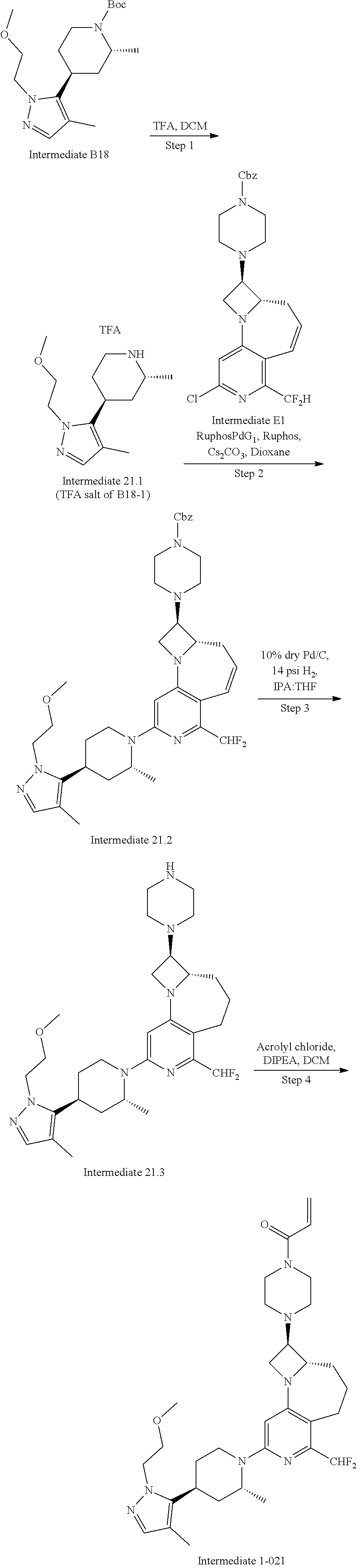

Step 1. (2R,4S)-4-(1-(2-Methoxyethyl)-4-methyl-1H-pyrazol-5-yl)-2-methylpiperidine trifluoroacetate, Intermediate 21.1. To a solution of Intermediate B18 (7 g, 20.74 mmol) in DCM (105 mL), was added TFA (21 mL, 270 mmol) dropwise at 0° C. The reaction mixture was stirred for 2 b at rt. The reaction mixture was concentrated, azeotroped with toluene (2×150 mL), washed with diethylether (2×150 mL), and dried to provide Intermediate 21.1 as a TFA salt (7 g, 20.94 mmol, 100% yield), m/z (ESI): 238.2 (M+H)⁺.

Step 2. Benzyl 4-((7aS,8R)-4-(difluoromethyl)-2-((2R,4S)-4-(1-(2-methoxyethyl)-4-methyl-1H-pyrazol-5-yl)-2-methylpiperidin-1-yl)-7,7a,8,9-tetrahydroazeto[1,2-a]pyrido[3,4-f]azepin-8-yl)piperazine-1-carboxylate, Intermediate 21.2. To a solution of Intermediate 21.1 (6.34 g, 18.95 mmol) in 1,4-dioxane (180 mL), was added Cs₂CO₃(41.2 g, 126 mmol) and stirred for 5 min. Then, Intermediate E1 (6.00 g, 12.63 mmol) was added, and the reaction mixture was purged by N₂ for 2 min. Then, RuPhos (0.589 g, 1.263 mmol) was added followed by RuPhos Pd G1 (1.032 g, 1,263 mmol). The reaction mixture was stirred for 4 h at 110° C. The reaction mixture was diluted with water (350 mL) and extracted with EtOAc (3×250 mL). The combined organic extracts were washed with brine (300 mL), dried over Na₂SO₄, filtered, and concentrated. The crude material was purified by chromatography, eluting with a gradient of 40% to 80% EtOAc in hexanes, to give Intermediate 21.2 (3.9 g, 5.77 mmol, 46% yield), m/z (ESI): 676.2 (M+H)⁺.

Step 3. (7aS,8R)-4-(Difluoromethyl)-2-((2R,4S)-4-(1-(2-methoxyethyl)-4-methyl-1H-pyrazol-5-yl)-2-methylpiperidin-1-yl)-8-(piperazin-1-yl)-5,6,7,7a,8,9-hexahydroazeto[1,2-a]pyrido[3,4-f]azepine, Intermediate 21.3. A solution of benzyl 4-((7aS,8R)-4-(difluoromethyl)-2-((2R,4S)-4-(1-(2-methoxyethyl)-4-methyl-1H-pyrazol-5-yl)-2-methylpiperidin-1-yl)-7,7a,8,9-tetrahydroazeto[1,2-a]pyrido[3,4-f]azepin-8-yl)piperazine-1-carboxylate (3.9 g, 5.77 mmol) in IPA (58.5 mL) and THF (58.5 mL) was purged with N₂ for 5 minutes. Then, palladium on activated carbon (10 wt %, 1.84 g, 1.73 mmol) was added, and the reaction mixture was degassed thoroughly and stirred under atmospheric H₂ pressure for 3 h at rt. The mixture was filtered through a bed of celite. washed with 15% MeOH in DCM (750 mL), and concentrated to provide crude Intermediate 21.3 that was taken to next step, m/z (ESI): 544.3 (M+H)⁺.

Step 4. 1-(4-((7aS,8R)-4-(Sifluoromethyl)-2-((2R,4S)-4-(1-(2-methoxyethyl)-4-methyl-1H-pyrazol-5-yl)-2-methyl-1-piperidinyl)-5,6,7,7a,8,9-hexahydroazeto[1,2-a]pyrido[3,4-f]azepin-8-yl)-1-piperazinyl)-2-propen-1-one, Compound 1-021. To a solution of Intermediate 21.3 (3.2 g, 5.89 mmol) in DCM (32.0 mL) was added DIPEA (5.14 mL, 29.4 mmol) dropwise at 0° C. After, acryloyl chloride (0.574 mL, 7.06 mmol) in 1 mL DCM was added at −78° C., and the reaction mixture was stirred for 20 min at −78° C. The reaction mixture was quenched by ice cold water (75 mL) and extracted with DCM (2×50 mL). The combined organic extract were dried over Na₂SO₄, filtered, and concentrated. The crude mixture was purified by RP-MPLC Buchi C18-120 g Column with a mobile phase of A: 0.01% ammonia in water B:-ACN using a flow rate of 20 mL/min to provide 1-(4-((7aS,8R)-4-(difluoromethyl)-2-((2R,4S)-4-(1-(2-methoxyethyl)-4-methyl-1H-pyrazol-5-yl)-2-methyl-1-piperidinyl)-5,6,7,7a,8,9-hexahydroazeto[1,2-a]pyrido[3,4-f]azepin-8-yl)-1-piperazinyl)-2-propen-1-one, Compound 1-021 (2.4 g, 37% yield), m/z (ESI): 598.2 (M+H)⁺. ¹H NMR (400 MHz, DMSO-d₆)₆ (ppm) 7.13 (s, 1H), 6.59-6.90 (m, 2H), 6.11 (dd, J=16.7, 2.4 Hz, 1H), 5.87 (s, 1H), 5.68 (dd, J=10.4, 2.4 Hz, 1H), 4.73 (br s, 1H), 4.08-4.38 (m, 3H), 3.43-3.80 (m, 9H), 3.29 (s, 1H), 3.16 (s, 3H), 2.80-3.07 (m, 3H), 2.39-2.27 (br s, 5H), 2.08-1.98 (m, 4H), 1.53-1.96 (m, 6H), 1.39 (d, J=12.1 Hz, 1H), 1.17 (d, J=6.7 Hz, 3H).

Compounds in Table 2-2 were prepared following the procedure described in Method 2, using appropriate starting materials. All starting materials are commercially available or are described in the Intermediates section above.

TABLE 2-2

| Compound | | LCMS: (ESI + ve ion) m/z; | |
|---|---|---|---|
| # | Chemical Structure & Name | NMR | Comments |
| 1-022 | 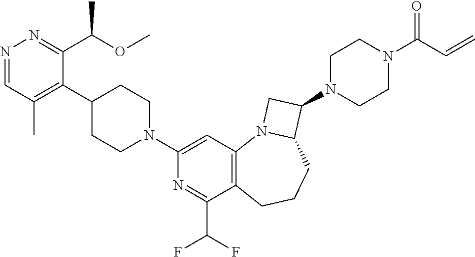 [SMILES: C=CC(=O)N1CCN([C@@H]2CN3c4cc(N5CCC(c6c(C)cnnc6[C@@H](C)OC)CC5)nc(C(F)F)c4CCC[C@@H]23)CC1] 1-(4-((7aS,8R)-4-(difluoromethyl)-2- (4-(3-((1R)-1-methoxyethyl)-5- methyl-4-pyridazinyl)-1-piperidinyl)- 5,6,7,7a,8,9-hexahydroazeto[1,2- a]pyrido[3,4-f]azepin-8-yl)-1- piperazinyl)-2-propen-1-one | m/z (ESI): 596.350 (M + H)⁺. ¹H NMR (400 MHz, MeOH- d₄) δ (ppm) 8.82-8.85 (m. 1 H), 6.77 (dd, J = 16.9, 10.6 Hz, 1 H), 6.39-6.66 (m, 1 H), 6.22 (dd, J = 16.8, 1.9 Hz, 1 H), 5.98-6.02 (m, 1 H), 5.75-5.78 (m, 1 H), 4.97-5.01 (m, 1 H), 4.45- 4.55 (m, 2 H), 3.60-3.89 (m, 10 H), 3.15-3.26 (m, 2 H), 2.90-3.02 (m, 3 H), 2.45-2.52 (m, 8 H), 2.18- 2.31 (m, 2 H), 1.81-2.08 (m, 4 H), 1.71-1.78 (m, 2 H), 1.62 (d, J = 1.0 Hz, 3 H).); ¹⁹F NMR (376 MHz, MeOH-d₄) δ (ppm) −112.44 (d, J = 21.7 Hz, 1 F). | Intermediate B43 was used; The stereochemistry of the pyridazine side chain was arbitrarily assigned |
| 1-023 | 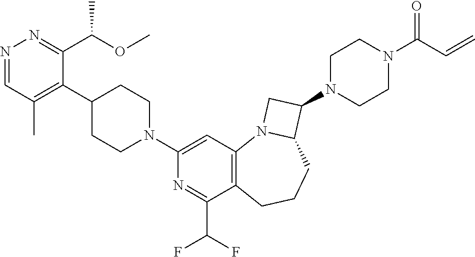 [SMILES: C=CC(=O)N1CCN([C@@H]2CN3c4cc(N5CCC(c6c(C)cnnc6[C@H](C)OC)CC5)nc(C(F)F)c4CCC[C@@H]23)CC1] 1-(4-((7aS,8R)-4-(difluoromethyl)-2- (4-(3-((1S)-1-methoxyethyl)-5- methyl-4-pyridazinyl)-1-piperidinyl)- 5,6,7,7a,8,9-hexabydroazeto[1,2- a]pyrido[3,4-f]azepin-8-yl)-1- piperazinyl)-2-propen-1-one | m/z (ESI): 596.350 (M + H)⁺. ¹H NMR (400 MHz, MeOH- d4) δ (ppm) 8.83 (s, 1 H), 6.37-6.86 (m, 2 H), 6.18- 6.26 (m, 1 H), 5.97 (s, 1 H), 5.74-5.80 (m, 1 H), 4.99 (br d, J = 7.0 Hz, 1 H), 4.50 (br t, J = 11.2 Hz, 2 H), 3.61- 3.91 (m, 9 H), 3.20 (br dd, J = 13.8, 9.0 Hz, 1 H), 2.88- 3.03 (m, 3 H), 2.48 (s, 9 H), 2.23 (br dd, J = 12.4. 3.3 Hz, 2 H), 1.83-2.08 (m, 3 H), 1.74 (br t, J = 11.4 Hz, 2 H), 1.62 (br d, J = 6.4 Hz, 3 H), 1.29-1.55 (m, 2 H). ¹⁹F NMR (376 MHz, MeOH-d4) δ (ppm) −112.42 (d, J = 25.1 Hz, 1 F) | Intermediate B44 was used; The stereochemistry of the pyridazine side chain was arbitrarily assigned |
| 1-024 | 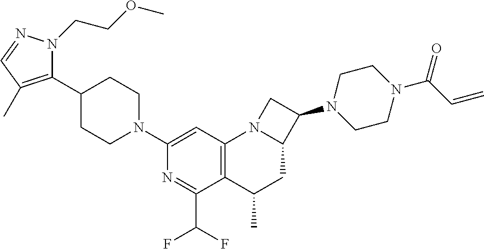 [SMILES: C=CC(=O)N1CCN([C@@H]2CN3c4cc(N5CCC(c6c(C)cnn6CCOC)CC5)nc(C(F)F)c4[C@@H](C)C[C@@H]23)CC1] 1-(4-((5S,6aS,7R)-4- (difluoromethyl)-2-(4-(1-(2- methoxyethyl)-4-methyl-1H-pyrazol- 5-yl)-1-piperidinyl)-5-methyl- 6,6a,7,8-tetrahydro-5H-azeto[1,2- a][1,6]naphthyridin-7-yl)-1- piperazinyl)-2-propen-1-one | m/z (ESI): 584.3 (M + H)⁺. ¹H NMR (400 MHz, DMSO-d₆) δ (ppm) 7.13 (s, 1H), 6.98-6.67 (m, 2H), 6.12 (dd, J = 16.7, 2.4 Hz, 1H), 5.78 (s, 1H), 5.69 (dd, J = 10.4, 2.4 Hz, 1H), 4.45- 4.27 (m, 2H), 4.21 (t, J = 5.4 Hz. 2H), 4.16-4.00 (m, 2H), 3.91 (t, J = 8.4 Hz, 1H), 3.61 (t, J = 5.4 Hz, 6H), 3.21 (s, 3H), 3.01 (dt, J = 10.0, 5.3 Hz, 2H), 2.84 (br s, 1H), 2,72 (d, J = 10.1 Hz, 2H), 2.35 (br s, 4H), 2.09-2.01 (m, 1H), 1.99 (s, 3H), 1.88-1.67 (m, 4H), 1.42 (q, J = 11.9 Hz, 1H), 1.31 (d, J = 6.5 Hz, 3H). | Intermediates E11 and B21 were used; |
| 1-025 | 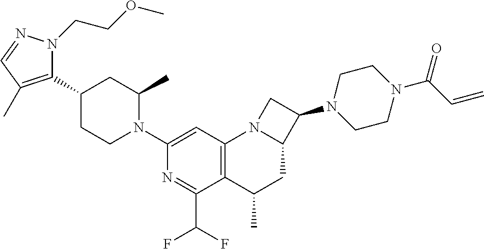 [SMILES: C=CC(=O)N1CCN([C@@H]2CN3c4cc(N5CC[C@H](c6c(C)cnn6CCOC)C[C@H]5C)nc(C(F)F)c4[C@@H](C)C[C@@H]23)CC1] 1-(4-((5S,6aS,7R)-4- (difluoromethyl)-2-((2R,4S)-4-(1-(2- methoxyethyl)-4-methyl-1H-pyrazol- 5-yl)-2-methyl-1-piperidinyl)-5- methyl-6,6a,7,8-tetrahydro-5H- azeto[1,2-a][1,6]naphthyridin-7-yl)-1- piperazinyl)-2-propen-1-one | m/z (ESI): 598.3 (M + H)⁺. ¹H NMR (400 MHz, DMSO-d₆) δ (ppro) 7.12 (s, 1H), 6.97-6.67 (m, 2H), 6.12 (dd, J = 16.7, 2.4 Hz, 1H), 5.73-5.65 (m, 2H). 4.60 (br s, 1H), 4.36-4.24 (m, 2H), 4.24-4.10 (m), 2H), 4.05 (dt, J = 11.6, 4.3 Hz, 1H), 3.91 (t, J = 8.4 Hz, 1H), 3.69-3.54 (m, 6H), 3.22 (s, 3H), 3.02-2.81 (m, 3H), 2.36 (d, J = 5.9 Hz, 5H), 2.01 (s, 5H), 1.76 (br s, 2H), 1.63 (d, J = 12.8 Hz, 1H), 1.42 (q, J = 11.9 Hz, 1H), 1.31 (d, J = 6.5 Hz, 3H), 1.15 (d, J = 6.8 Hz, 3H). | Intermediates E11 and B18 were used; |
| 1-026 | 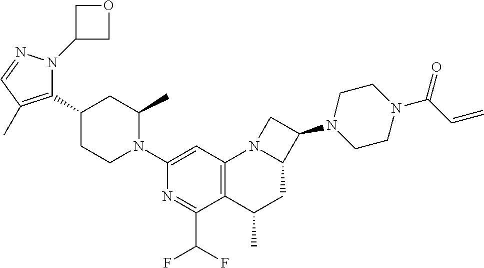 [SMILES: C=CC(=O)N1CCN([C@@H]2CN3c4cc(N5CC[C@H](c6c(C)cnn6C6COC6)C[C@H]5C)nc(C(F)F)c4[C@@H](C)C[C@@H]23)CC1] 1-(4-((5S,6aS,7R)-4- (difluoromethyl)-5-methyl-2- ((2R,4S)-2-methyl-4-(4-methyl-1-(3- oxetanyl)-1H-pyrazol-5-yl)-1- piperidinyl)-6,6a,7,8-tetrahydro-5H- azeto[1,2-a][1,6]naphthyridin-7-yl)-1- piperazinyl)-2-propen-1-one | m/z (ESI): 596.4 (M + H)⁺. ¹H NMR (400 MHz, DMSO-d₆) δ (ppm) 7.29 (s, 1H), 6.97-6.67 (m, 2H), 6.12 (dd, J = 16.7, 2.4 Hz, 1H), 5.74-5.65 (m, 3H), 5.01-4.84 (m, 4H), 4.58 (br s, 1H), 4.28 (d, J = 13.2 Hz, 1H), 4.13 (dd, J = 9.0, 5.1 Hz, 1H), 4.05 (dt, J = 11.6, 4.3 Hz, 1H), 3.91 (t, J = 8.4 Hz, 1H), 3.59 (d, J = 13.3 Hz, 4H), 3.15 (br s, 1H), 3.04-2.79 (m, 3H), 2.36 (d, J = 6.1 Hz, 4H), 2.09-1.94 (m, 5H), 1.73 (dd, J = 17.8, 5.6 Hz, 2H), 1.55 (d, J = 13.0 Hz, 1H), 1.49-1.39 (m, 1H), 1.31 (d, J = 6.5 Hz, 3H), 1.17 (d, J = 6.8 Hz, 3H). | Intermediates E11 and B17 were used |
| 1-027 | 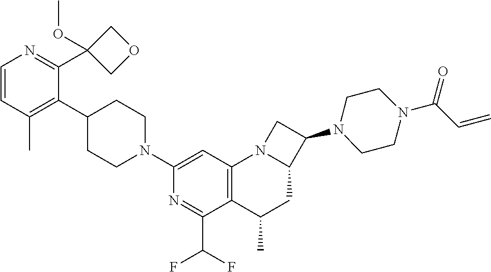 [SMILES: C=CC(=O)N1CCN([C@@H]2CN3c4cc(N5CCC(c6c(C)ccnc6C6(OC)COC6)CC5)nc(C(F)F)c4[C@@H](C)C[C@@H]23)CC1] 1-(4-((5S,6aS,7R)-4- (difluoromethyl)-2-(4-(2-(3-methoxy- 3-oxetanyl)-4-methyl-3-pyridinyl)-1- piperidinyl)-5-methyl-6,6a,7,8- tetrahydro-5H-azeto[1,2- a][1,6]naphthyridin-7-yl)-1- piperazinyl)-2-propen-1-one | m/z (ESI): 623.3 (M + H)⁺. ¹H NMR (400 MHz, DMSO-d₆) δ (ppm) 8.28 (d, J = 4.9 Hz, 1H), 7.17 (d, J = 5.0 Hz, 1H), 7.00-6.66 (m, 2H), 6.12 (dd, J = 16.7, 2.5 Hz, 1H), 5.79 (s, 1H), 5.69 (dd, J = 10.4, 2.4 Hz, 1H), 5.14 (d, J = 7.1 Hz, 2H), 4.80 (d, J = 7.1 Hz, 2.H), 4.34 (t, J = 15.5 Hz, 2H), 4.18-4.02 (m, 2H), 3.91 (t, J = 8.4 Hz, 1H), 3.59 (d, J = 12.3 Hz, 4H), 3.01 (dt, J = 9.2, 4.9 Hz, 1H), 2.95 (s, 3H), 2.84 (br s, 1H), 2.75-2.60 (m, 3H), 2.37 (d, J = 13.0 Hz, 7H), 2.15-1.97 (m, 3H), 1.57 (d, J = 12.6 Hz, 2H), 1.42 (q, J = 11.9 Hz, 1H), 1.31 (d, J = 6.4 Hz, 3H). | Intermediates E11 and B1 were used |
| 1-028 | 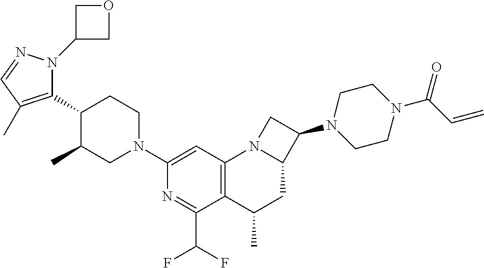 [SMILES: C=CC(=O)N1CCN([C@@H]2CN3c4cc(N5CC[C@@H](c6c(C)cnn6C6COC6)[C@H](C)C5)nc(C(F)F)c4[C@@H](C)C[C@@H]23)CC1] 1-(4-((5S,6aS,7R)-4- (difluoromethyl)-5-methyl-2- ((3S,4R)-3-methyl-4-(4-methyl-1-(3- oxctanyl)-1H-pyrazol-5-yl)-1- piperidinyl)-6,6a,7,8-tetrahydro-5H- azeto[1,2-a][1,6]naphthyridin-7-yl)-1- piperazinyl)-2-propen-1-one | m/z (ESI): 596.0 (M + H)⁺. ¹H NMR (400 MHz, CDCl₃) δ (ppm) 7.38 (s, 1 H), 6.42-6.75 (m, 2 H), 6.30 (dd, J = 16.7, 1.9 Hz, 1 H), 5.68-5.75 (m, 1 H), 5.64 (s, 1 H), 5.48 (quin, J = 7.1 Hz, 1 H), 5.23 (t, J = 6.4 Hz, 1 H), 5.17 (t, J = 6.4 Hz, 1 H), 4.87-4.95 (m, 2 H), 4.41 (br dd, J = 13.0, 2.3 Hz, 1 H), 4.26- 4.35 (m, 1 H), 4.21 (dd, J = 8.6, 5.2 Hz, 1 H), 4.11 (dt, J = 11,5, 4.4 Hz, 1 H), 3.94 (t, J = 8.2 Hz, 1 H), 3.59- 3.80 (m, 4 H), 3.05 (dt, J = 7.6, 4.8 Hz, 1 H), 2.95 (br dd, J = 6.0, 4.5 Hz, 1 H), 2.73 (td, J = 12.5, 2.6 Hz, 1 H), 2.45 (br s, 4 H), 2.31- 2.42 (m, 2 H), 2.01-2.12 (m, 5 H), 1.85-1.99 (m, 1 H), 1.69 (br dd, J = 13.2, 2.7 Hz, 1 H), 1.54 (q, J = 11.7 Hz, 1 H), 1.44 (d, J = 6.5 Hz, 3 H), 0.72 (d, J = 6.7 Hz, 3 H) ¹⁹F NMR (376 MHz, CDCl₃) δ (ppm) −123.25- −106.72 (m, 2 F) | Intermediates E11 and B24 were used Step 3. EtOH was used as solvent Step 4. Acrylic anhydride, Na₂CO₃ was used |
| 1-029 | 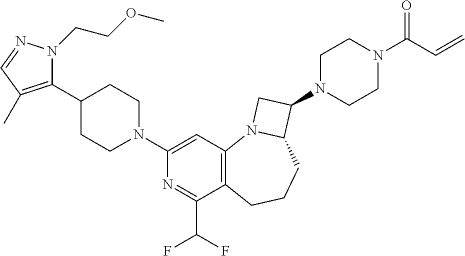 [SMILES: C=CC(=O)N1CCN([C@@H]2CN3c4cc(N5CCC(c6c(C)cnn6CCOC)CC5)nc(C(F)F)c4CCC[C@@H]23)CC1] 1-(4-((7aS,8R)-4-(difluoromethyl)-2- (4-(1-(2-methoxyethyl)-4-methyl-1H- pyrazol-5-yl)-1-piperidinyl)- 5,6,7,7a,8,9-hexahydroazeto[1,2- a]pyrido[3,4-f]azepin-8-yl)-1- piperazinyl)-2-propen-1-one | m/z (ESI): 584.4 (M + H)⁺. ¹H NMR (400 MHz, DMSO-d₆) δ (ppm) 7.13 (s, 1H), 6.87-6.55 (m, 2H), 6.11 (dd, J = 16.7, 2.4 Hz, 1H), 5.94 (s, 1H), 5.68 (dd, J = 10.4, 2.4 Hz, 1H), 4.42 (t, J = 10.9 Hz, 2H), 4.22 (t, J = 5.4 Hz, 2H), 3.77-3.68 (m, 3H), 3.65-3.48 (m, 6H), 3.21 (s, 3H), 3.02 (dd, J = 8.5, 4.9 Hz, 2H), 2.90 (q, J = 6.7 Hz, 1H), 2.76 (t, J = 12.6 Hz, 2H), 2.34 (dd, J = 13.7, 5.3 Hz, 5H), 1.99 (s, 3H), 1.82 (dq, J = 32.8, 14.3, 12.6 Hz, 7H), 1.40 (d, J = 11.8 Hz, 1H). | Intermediate B21 was used; Step 3. MeOH was used as solvent |
| 1-030 | 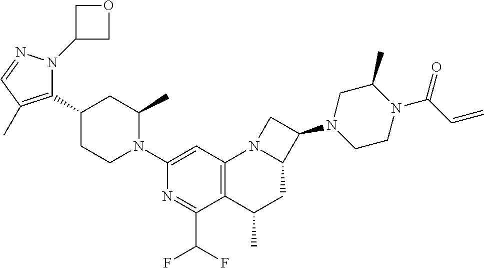 [SMILES: C=CC(=O)N1CCN([C@@H]2CN3c4cc(N5CC[C@H](c6c(C)cnn6C6COC6)C[C@H]5C)nc(C(F)F)c4[C@@H](C)C[C@@H]23)C[C@H]1C] 1-((2R)-4-((5S,6aS,7R)-4- (difluoromethyl)-5-methyl-2- ((2R,4S)-2-methyl-4-(4-methyl-1-(3- oxetanyl)-1H-pyrazol-5-yl)-1- pipcridinyl)-6,6a,7,8-tetrahydro-5H- azeto[1,2-a][1,6]naphthyridin-7-yl)-2- methyl-1-piperazinyl)-2-propen-1-one | m/z (ESI): 610.2(M + H)⁺. ¹H NMR (400 MHz, DMSO-d₆) δ (ppm) 7.29 (s, 1H), 6.97-6.66 (m, 2H), 6.11 (dd, J = 16.6, 2.4 Hz, 1H), 5.74-5.63 (m, 3H), 4.98 (t, J = 6.2 Hz, 1H), 4.89 (dq, J = 5.9, 2.2, 1.2 Hz, 3H), 4.57 (s, 2H), 4.29 (d, J = 13.5 Hz, 2H), 4.06 (ddd, J = 15.2, 8.7, 4.7 Hz, 2H), 3.90 (t, J = 8.4 Hz, 1H), 3.19-3.10 (m, 1H), 3.01-2.77 (m, 5H), 2.71- 2.62 (m, 1H), 2.08-1.93 (m, 6H), 1.85 (d, J = 10.8 Hz, 1H), 1.77-1.64 (m, 2H), 1.55 (d, J = 13.3 Hz, 1H), 1.43 (q, J = 12.0 Hz, 1H), 1.31 (d, J = 6.4 Hz, 3H), 1.25 (s, 3H), 1.17 (d, J = 6.7 Hz, 3H). | Intermediates E12 and B17 were used; Step 3. MeOH was used as solvent |
| 1-031 | 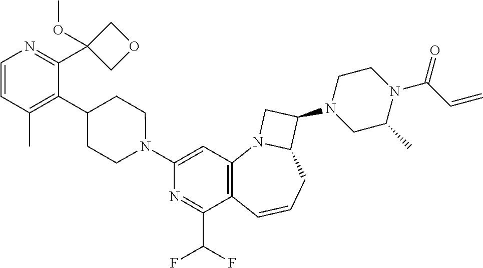 [SMILES: C=CC(=O)N1CCN([C@@H]2CN3c4cc(N5CCC(c6c(C)ccnc6C6(OC)COC6)CC5)nc(C(F)F)c4C=CC[C@@H]23)C[C@H]1C] 1-((2R)-4-((5S,6aS,7R)-4- (difluoromethyl)-2-(4-(2-(3-methoxy- 3-oxetanyl)-4-methyl-3-pyridinyl)-1- piperidinyl)-5-methyl-6,6a,7,8- tetrahydro-5H-azeto[1,2- a][1,6]naphthyridin-7-yl)-2-methyl-1- piperazinyl)-2-propen-1-one | m/z (ESI): 637.3 (M + H)⁺. ¹H NMR (400 MHz, DMSO-d₆) δ (ppm) 8.28 (d, J = 4.9 Hz, 1H), 7.17 (d, J = 5.0 Hz, 1H), 7.00-6.69 (m, 2H), 6.11 (dd, J = 16.7, 2.4 Hz, 1H), 5.80 (s, 1H), 5.68 (dd, J = 10.4, 2.4 Hz, 1H), 5.20-5.09 (m, 2H), 4.80 (d, J = 7.1 Hz, 2H), 4.44-4.17 (m, 3H), 4.17- 3.99 (m, 3H), 3.91 (t, J = 8.4 Hz, 1H), 2.99-2.79 (m, 7H), 2.75-2.60 (g, J = 12.4 Hz, 4H), 2.38 (s, 3H), 2.15-1.95 (m, 4H), 1.84 (br s, 1H), 1.56 (d, J = 12.6 Hz, 2H), 1.43 (q, J = 11.9 Hz, 1H), 1.32 (d, J = 6.4 Hz, 3H), 1.25 (s, 3H). | Intermediates E12 and B1 were used; Step 3. MeOH was used as solvent |
| 1-032 | 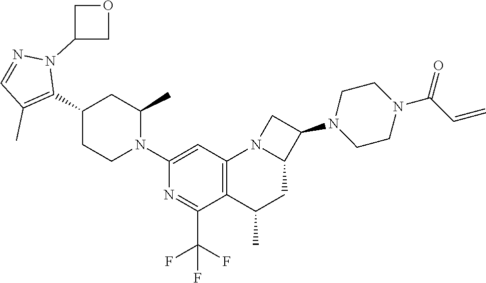 [SMILES: C=CC(=O)N1CCN([C@@H]2CN3c4cc(N5CC[C@H](c6c(C)cnn6C6COC6)C[C@H]5C)nc(C(F)(F)F)c4[C@@H](C)C[C@@H]23)CC1] 1-(4-((5S,6aS,7R)-5-methyl-2- ((2R,4S)-2-methyl-4-(4-methyl-1-(3- oxetanyl)-1H-pyrazol-5-yl)-1- piperidinyl)-4-(trifluoromethyl)- 6,6a,7,8-tetrahydro-5H-azeto[1,2- a][1,6]naphthyridin-7-yl)-1- piperazinyl)-2-propen-1-one | m/z (ESI): 614.3 (M + H)⁺. ¹H NMR (400 MHz, DMSO-d₆) δ (ppm) 7.29 (s, 1H), 6.82 (dd, J = 16.7, 10.4 Hz, 1H), 6.12 (dd, J = 16.7, 2.4 Hz, 1H), 5.77 (s, 1H), 5.74-5.64 (m, 2H), 4.97 (t, J = 6.2 Hz, 1H), 4.94-4.83 (m, 3H), 4.54 (br s, 1H), 4.26 (d, J = 13.4 Hz, 1H), 4.15 (dd, J = 9.1, 4.9 Hz, 1H), 4.09-3.90 (m, 2H), 3.59 (d, J = 12.7 Hz, 4H), 3.14 (d, J = 12.3 Hz, 1H), 3.06-2.80 (m, 3H), 2.37 (q, J = 5.9, 5.2 Hz, 4H), 2.09 (dt, J = 12.7, 4.9 Hz, 1H), 2.02 (s, 3H), 1.96 (dd, J = 13.2, 5.2 Hz, 1H), 1.74 (d, J = 18.8 Hz, 2H), 1.55 (d, J = 12.9 Hz, 1H), 1.45 (q, J = 12.0 Hz, 1H), 1.25 (d, J = 6.4 Hz, 3H), 1.18 (d, J = 6.7 Hz, 3H). | Intermediates E11 and B17 were used; Step 3. MeOH was used as solvent |
| 1-033 | 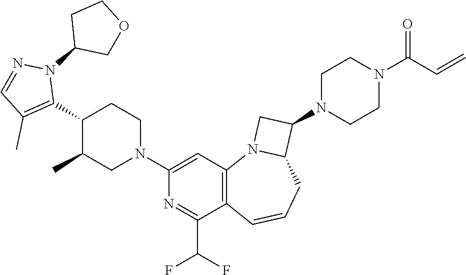 [SMILES: C=CC(=O)N1CCN([C@@H]2CN3c4cc(N5CC[C@@H](c6c(C)cnn6[C@H]6CCOC6)[C@H](C)C5)nc(C(F)F)c4C=CC[C@@H]23)CC1] 1-(4-((7aS,8R)-4-(difluoromethyl)-2- ((3S,4R)-3-methyl-4-(4-methyl-1- ((3S)-tetrahydro-3-furanyl)-1H- pyrazol-5-yl)-1-piperidinyl)- 5,6,7,7a,8,9-hexahydroazeto[1,2- a]pyrido[3,4-f]azepin-8-yl)-1- piperazinyl)-2-propen-1-one | m/z (ESI): 610.3 (M + H)⁺ ¹H NMR (DMSO-d₆, 400 MHz): δ (ppm) 7.21 (s, 1 H), 6.57-6.87 (m, 2 H), 6.11 (dd, J = 16.6, 2.4 Hz, 1 H), 5.95 (s, 1H), 5.68 (dd, J = 10.5, 2.4 Hz, 1 H), 5.12 (bs, 1 H), 4.37-4.47 (m, 2 H), 4.08-3.94 (m, 2 H), 3.84 (q, J = 7.1 Hz, 1 H), 3.77-3.67 (m, 4 H), 3.56 (d, J = 11.0 Hz, 4 H), 3.01 (t, J = 12.0 Hz, 1 H), 2.89 (q, J = 6.6 Hz, 1 H), 2.73-2.82 (m, 2 H), 2.39-2.16 (m, 6 H), 2.02 (s, 1 H), 1.96 (s, 3 H), 1.68-1.92 (m, 5 H), 1.41 (s, 1 H), 0.71 (d, J = 6.5 Hz, 3 H). | Intermediate B40 were used; |
| 1-034 | 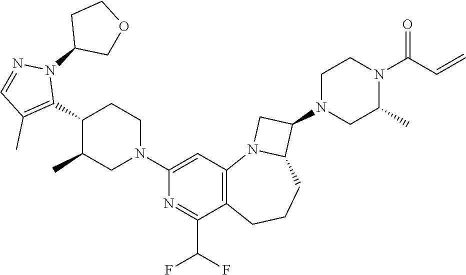 [SMILES: C=CC(=O)N1CCN([C@@H]2CN3c4cc(N5CC[C@@H](c6c(C)cnn6[C@H]6CCOC6)[C@H](C)C5)nc(C(F)F)c4CCC[C@@H]23)C[C@H]1C] 1-((2R)-4-((7aS,8R)-4- (difluoromethyl)-2-((3S,4R)-3- methyl-4-(4-methyl-1-((3S)- tetrahydro-3-furanyl)-1H-pyrazol-5- yl)-1-piperidinyl)-5,6,7,7a,8,9- hexahydroazeto[1,2-a]pyrido[3,4- f]azepin-8-yl)-2-methyl-1- piperazinyl)-2-propen-1-one | m/z (ESI): 624.3 (M + H)⁺. ¹H NMR (400 MHz, DMSO-d₆) δ (ppm) 7.21 (s, 1H), 6.87-6.55 (m, 2H), 6.10 (dd, J = 16.8, 2.5 Hz, 1H), 5.92 (s, 1H), 5.68 (dd, J = 10.4, 2.4 Hz, 1H), 5.12 (s, 1H), 4.49-4.30 (m, 3H), 4.01 (dt, J = 27.9, 7.7 Hz, 3H), 3.91-3.69 (m, 5H), 3.63 (t, J = 7.3 Hz, 1H), 3.00 (t, J = 12.1 Hz, 2H), 2.88-2.57 (m, 5H), 2.46-2.30 (m, 3H), 2.24 (s, 2H), 2.03 (s, 3H), 1.93- 1.67 (m, 6H), 1.44 (s, 1H), 1.23 (s, 3H), 0.71 (d, J = 6.5 Hz, 3H). | Intermediates E2 and B40 were used; Step 3. THF and MeOH were used as solvents |
| 1-035 | 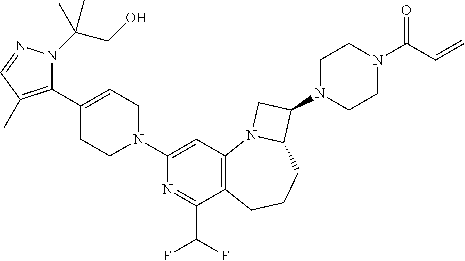 [SMILES: C=CC(=O)N1CCN([C@@H]2CN3c4cc(N5CC=C(c6c(C)cnn6C(C)(C)CO)CC5)nc(C(F)F)c4CCC[C@@H]23)CC1] 1-(4-((7aS,8R)-4-(difluoromethyl)-2- (4-(1-(1-hydroxy-2-methyl-2- propanyl)-4-methyl-1H-pyrazol-5-yl)- 3,6-dihydro-1(2H)-pyridinyl)- 5,6,7,7a,8,9-hexahydroazeto[1,2- a]pyrido[3,4-f]azepin-8-yl)-1- piperazinyl)-2-propen-1-one | m/z (ESI): 596.00 (M + H)⁺. ¹H NMR (400 MHz, DMSO-d₆) δ (ppm) 7.20 (s, 1 H), 6.57-6.89 (m, 2 H), 6.11 (dd, J = 16.7, 2.2 Hz, 1 H), 5.92 (s, 1 H), 5.85 (br s, 1 H), 5.65-5.72 (m, 1 H), 4.83 (t, J = 5.9 Hz, 1 H), 4.05 (br s, 2 H), 3.68-3.82 (m, 5 H), 3.51-3.63 (m, 6 H), 2.97-3.08 (m, 1 H), 2.91 (br d, J = 6.2 Hz, 1 H), 2.27- 2.39 (m, 7 H), 1.92-1.73 (m, 7 H), 1.45 (s. 6 H). | Intermediate B33 was used; |
| 1-036 | 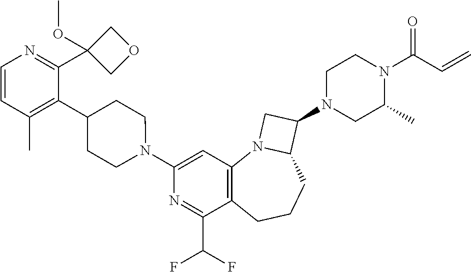 [SMILES: C=CC(=O)N1CCN([C@@H]2CN3c4cc(N5CCC(c6c(C)ccnc6C6(OC)COC6)CC5)nc(C(F)F)c4CCC[C@@H]23)C[C@H]1C] 1-((2R)-4-((7aS,8R)-4- (difluoromethyl)-2-(4-(2-(3-methoxy- 3-oxetanyl)-4-methyl-3-pyridinyl)-1- piperidinyl)-5,6,7,7a,8,9- hexahydroazeto[1,2-a]pyrido[3,4- f]azepin-8-yl)-2-methyl-1- piperazinyl)-2-propen-1-one | m/z (ESI): 637.3 (M + H)⁺. ¹H NMR (400 MHz, DMSO-d₆) δ (ppm) 8.29 (d, J = 4.9 Hz, 1H), 7.18 (d, J = 5.0 Hz, 1H), 6.87-6.55 (m, 2H), 6.15-5.63 (m, 3H), 5.14 (d, J = 7.1 Hz, 2H), 4.81 (d, J = 7.1 Hz, 2H), 4.41 (t, J = 14.4 Hz, 3H), 4.21 (s, 1H), 3.76 (dt, J = 15.4, 6.9 Hz. 2H), 3.62 (t, J = 7.3 Hz, 1H), 3.08- 2.98 (m, 1H), 2.96 (s, 3H), 2.83 (q, J = 6.7 Hz, 2H), 2.78-2.57 (m, 5H), 2.39 (s, 4H), 2.07 (m, 3H), 1.96- 1.68 (m, 4H), 1.59 (d, J = 12.1 Hz, 2H), 1.45 (s, 1H), 1.22 (s, 3H). | Intermediates E2 and B1 were used |
| 1-037 | 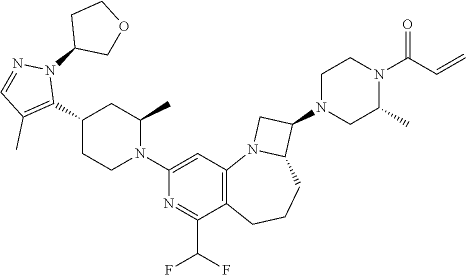 [SMILES: C=CC(=O)N1CCN([C@@H]2CN3c4cc(N5CC[C@H](c6c(C)cnn6[C@H]6CCOC6)C[C@H]5C)nc(C(F)F)c4CCC[C@@H]23)C[C@H]1C] 1-((2R)-4-((7aS,8R)-4- (difluoromethyl)-2-((2R,4S)-2- methyl-4-(4-methyl-1-((3S)- tetrahydro-3-furanyl)-1H-pyrazol-5- yl)-1-piperidinyl)-5,6,7,7a,8,9- hexahydroazeto[1,2-a]pyrido[3,4- f]azepin-8-yl)-2-methyl-1- piperazinyl)-2-propen-1-one | m/z (ESI): 624.4 (M + H)+. ¹H NMR (400 MHz, DMSO-d₆) δ (ppm) 7.17 (s, 1H), 6.84-6.54 (m, 2H), 6.10 (dd, J = 16.6, 2.4 Hz, 1H), 5.84 (s, 1H), 5.67 (dd, J = 10.4, 2.4 Hz, 1H), 5.12 (tt, J = 8.5. 4.8 Hz, 1H), 4.75 (br s, 1H), 4.59-4.12 (m, 3H), 4.07 (dd, J = 8.7, 7.0 Hz, 1H), 3.96 (q, J = 7.5 Hz, 1H), 3.88-3.70 (m, 4H), 3.61 (t, J = 7.2 Hz, 1H), 3.48-3.39 (m, 1H), 3.04-2.93 (m, 2H), 2.82 (q, J = 6.2 Hz, 2H), 2.63 (d, J = 11.3 Hz, 1H), 2.44- 2.24 (m, 3H), 2.24-2.15 (m, 1H), 2.02 (m, 5H), 1.94- 1.71 (m, 6H), 1.65 (d, J = 12.6 Hz, 1H), 1.45 (d, J = 11.1 Hz, 1H), 1.20 (t, J = 8.5 Hz, 6H). | Intermediates E2 and B19 were used |
| 1-038 | 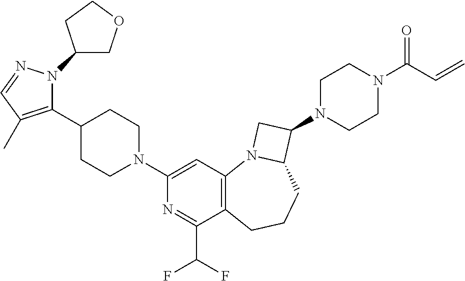 [SMILES: C=CC(=O)N1CCN([C@@H]2CN3c4cc(N5CCC(c6c(C)cnn6[C@H]6CCOC6)CC5)nc(C(F)F)c4CCC[C@@H]23)CC1] 1-(4-((7aS,8R)-4-(difluoromethyl)-2- (4-(4-methyl-1-((3S)-tetrahydro-3- furanyl)-1H-pyrazol-5-yl)-1- piperidinyl)-5,6,7,7a,8,9- hexahydroazeto[1,2-a]pyrido[3,4- f]azepin-8-yl)-1-piperazinyl)-2- propen-1-one | m/z (ESI): 596.3 (M + H)⁺. ¹H NMR (400 MHz, DMSO-d6) δ (ppm) 7.17 (s, 1H), 6.86-6.55 (m, 2H), 6.10 (dd, J = 16.6, 2.4 Hz, 1H), 5.92 (s, 1H), 5.67 (dd, J = 10.4, 2.4 Hz, 1H), 5.10 (tt, J = 7.6, 4.9 Hz, 1H), 4.42 (m, 2H), 4.07-3.94 (m, 2H), 3.88-3.72 (m, 5H), 3.62-3.42 (m, 6H), 3.16-2.96 (m, 2H), 2.93- 2.76 (m, 3H), 2.42-2.19 (m, 5H), 1.99 (s, 3H), 1.81 (tdd, J = 25.2, 19.6, 14.8 Hz, 7H), 1.44 (d, J = 9.0 Hz, 1H), | Intermediate B25 was used |
| 1-039 | 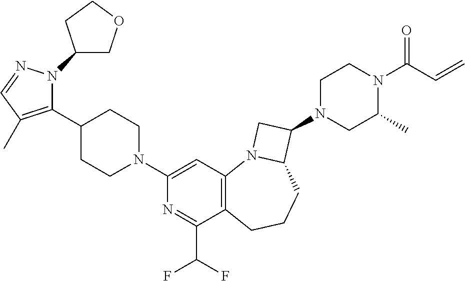 [SMILES: C=CC(=O)N1CCN([C@@H]2CN3c4cc(N5CCC(c6c(C)cnn6[C@H]6CCOC6)CC5)nc(C(F)F)c4CCC[C@@H]23)C[C@H]1C] 1-((2R)-4-((7aS,8R)-4- (difluoromethyl)-2-(4-(4-methyl-1- ((3S)-tetrahydro-3-furanyl)-1H- pyrazol-5-yl)-1-piperidinyl)- 5,6,7,7a,8,9-hexahydroazeto[1,2- a]pyrido[3,4-f]azepin-8-yl)-2-methyl- 1-piperazinyl)-2-propen-1-one | m/z (ESI): 610.3 (M + H)⁺. ¹H NMR (400 MHz, DMSO-d₆) δ (ppm) 7.17 (s, 1H), 6.86-6.55 (m, 2H), 6.10 (dd, J = 16.6, 2.4 Hz, 1H), 5.92 (s, 1H), 5.67 (dd, J = 10.4, 2.4 Hz, 1H), 5.10 (tt, J = 7.6, 4.9 Hz, 1H), 4.42 (t, J = 10.9 Hz, 2H), 4.07-3.94 (m, 2H), 3.88 3.72 (m, 5H), 3.62 (t, J = 7.3 Hz, 1H), 3.16-2.96 (m, 3H), 2.81 (td, J = 13.0, 8.2 Hz, 5H), 2.62 (d, J = 11.2 Hz, 1H), 2.42-2.19 (m, 3H), 2.10-1.96 (s, 4H), 1.81 (tdd, J = 25.2, 19.6, 14.8 Hz, 8H), 1.44 (d, J = 9.0 Hz, 1H), 1.22 (s, 3H). | Intermediates E2 and B25 were used |
| 1-040 | 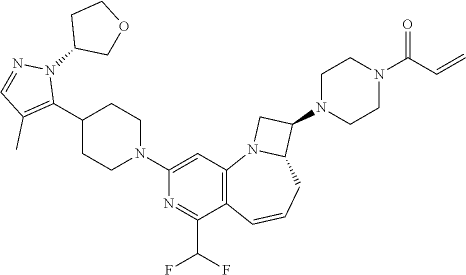 [SMILES: C=CC(=O)N1CCN([C@@H]2CN3c4cc(N5CCC(c6c(C)cnn6[C@@H]6CCOC6)CC5)nc(C(F)F)c4C=CC[C@@H]23)CC1] 1-(4-((7aS,8R)-4-(difluoromethyl)-2- (4-(4-methyl-1-((3R)-tetrahydro-3- furanyl)-1H-pyrazol-5-yl)-1- piperidinyl)-5,6,7,7a,8,9- hexahydroazeto[1,2-a]pyrido[3,4- f]azopin-8-yl)-1-piperazinyl)-2- propen-1-one | m/z (ESI): 596.3 (M + H)⁺. ¹H NMR (400 MHz, DMSO-d₆) δ (ppm) 7.17 (s, 1H), 6.86-6.53 (m, 2H), 6.11 (dd, J = 16.7, 2.4 Hz, 1H), 5.94 (s, 1H), 5.68 (dd, J = 10.4, 2.4 Hz, 1H), 5.15- 5.03 (m, 1H), 4.42 (m, 2H), 4.13-3.90 (m, 2H), 3.84 (td, J = 7.8, 5.7 Hz, 1H), 3.78-3.66 (m, 4H), 3.56 (d, J = 12.9 Hz, 4H), 3.06 (m, 2H), 2.90 (q, J = 6.6 Hz, 1H), 2.80 (t, J = 12.2 Hz, 2H), 2.34-2.21 (m, 6H), 1.99 (s, 3H), 1.92- 1.65 (m, 8H), 1.38 (d, J = 17.8 Hz, 1H). | Intermediate B26 was used |
| 1-041 | 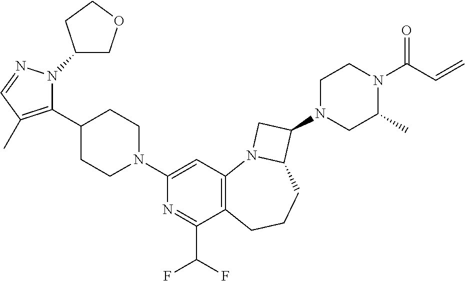 [SMILES: C=CC(=O)N1CCN([C@@H]2CN3c4cc(N5CCC(c6c(C)cnn6[C@@H]6CCOC6)CC5)nc(C(F)F)c4CCC[C@@H]23)C[C@H]1C] 1-((2R)-4-((7aS,8R)-4- (difluoromethyl)-2-(4-(4-methyl-1- ((3R)-tetrahydro-3-furanyl)-1H- pyrazol-5-yl)-1-piperidinyl)- 5,6,7,7a,8,9-hexahydroazeto[1,2- a]pyrido[3,4-f]azepin-8-yl)-2-methyl- 1-piperazinyl)-2-propen-1-one | m/z (ESI): 610.4 (M + H)⁺. ¹H NMR (400 MHz, DMSO-d₆) δ 7.17 (s, 1H), 6.85-6.56 (m, 2H), 6.10 (dd, J = 16.6, 2.4 Hz, 1H), 5.92 (s, 1H), 5.67 (dd, J = 10.5, 2.4 Hz, 1H), 5.10 (tt, J = 7.6, 4.9 Hz, 1H), 4.42 (t, J = 12.6 Hz, 2H), 4.09- 3.93 (m, 2H), 3.84 (dd, J = 7.8, 5.8 Hz, 2H), 3.75 (td, J = 8.2, 4.8 Hz, 3H), 3.62 (t, J = 7.3 Hz, 1H), 3.17-2.96 (m, 3H), 2.88-2.75 (m, 5H), 2.62 (d, J = 11.2 Hz, 1H), 2.42-2.20 (m, 3H), 2.09-1.97 (m, 4H), 1.95- 1.68 (m, 8H), 1.43 (s, 1H), 1.22 (s, 3H). | Intermediates E2 and B26 were used |
| 1-042 | 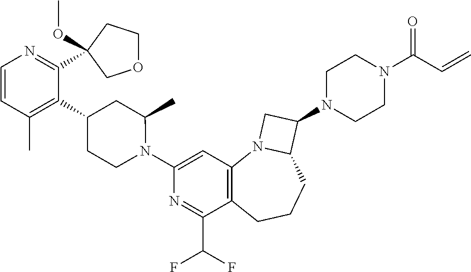 [SMILES: C=CC(=O)N1CCN([C@@H]2CN3c4cc(N5CC[C@H](c6c(C)ccnc6[C@]6(OC)CCOC6)C[C@H]5C)nc(C(F)F)c4CCC[C@@H]23)CC1] 1-(4-((7aS,8R)-4-(difluoromethyl)-2- ((2R,4S)-4-(2-((3R)-3- methoxytetrahydro-3-furanyl)-4- methyl-3-pyridinyl)-2-methyl-1- piperidinyl)-5,6,7,7a,8,9- hexahydroazeto[1,2-a]pyrido[3,4- f]azepin-8-yl)-1-piperazinyl)-2- propen-1-one | m/z (ESI): 651.3 (M + H)⁺. ¹H NMR (400 MHz, DMSO-d₆) δ (ppm) 8.22 (d, J = 4.8 Hz, 1H), 7.13 (d, J = 5.0 Hz, 1H), 6.89-6.52 (m, 2H), 6.11 (dd, J = 16.7, 2.4 Hz, 1H), 5.88 (s, 1H), 5.68 (dd, J = 10.4, 2.4 Hz, 1H), 4.78 (br s, 1H), 4.32 (d, J = 13.1 Hz, 1H), 4.25- 4.12 (m, 2H), 4.09-3.91 (m, 2H), 3.86-3.63 (m, 4H), 3.56 (m, 4H), 3.08- 2.80 (m, 6H), 2.70-2.54 (m, 1H), 2.46 (s, 3H), 2.49- 2.19 (m, 8H), 2.18-2.00 (m, 1H), 1.97-1.59 (m, 4H), 1.50 (d, J = 12.8 Hz, 1H), 1.46-1.31 (m, 1H), 1.19 (d, J = 6.7 Hz, 3H) | Intermediate B2 was used; After Step 1, the sample was purified via SFC using a CHIRALPAK IC (150 × 50) mm, 5 μm, column with a mobile phase of liquid CO₂: [ACN:IPA(1:1)] (50:50)] using a flowrate of 200 mL/min (2nd eluting isomer) |
| 1-043 | 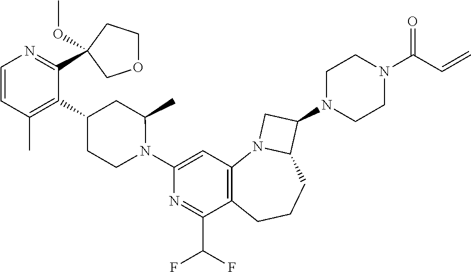 [SMILES: C=CC(=O)N1CCN([C@@H]2CN3c4cc(N5CC[C@H](c6c(C)ccnc6[C@@]6(OC)CCOC6)C[C@H]5C)nc(C(F)F)c4CCC[C@@H]23)CC1] 1-(4-((7aS,8R)-4-(difluoromethyl)-2- ((2R,4S)-4-(2-((3S)-3- methoxytetrahydro-3-furanyl)-4- methyl-3-pyridinyl)-2-methyl-1- piperidinyl)-5,6,7,7a,8,9- hexahydroazeto[1,2-a]pyrido[3,4- f]azepin-8-yl)-1-piperazinyl)-2- propen-1-one | m/z (ESI): 651.3 (M + H)⁺. ¹H NMR (400 MHz, DMSO-d₆) δ (ppm) 8.24 (d, J = 4.9 Hz, 1H), 7.13 (d, J = 4.9 Hz, 1H), 6.86-6.52 (m, 2H), 6.11 (dd, J = 16.7, 2.4 Hz, 1H), 5.88 (s, 1H), 5.68 (dd, J = 10.4, 2.4 Hz, 1H), 4.79 (br s, 1H), 4.31 (d, J = 13.0 Hz, 1H), 4.23 (d, J = 9.6 Hz, 1H), 4.09 (d, J = 9.6 Hz, 1H), 4.03-3.86 (m, 2H), 3.80 (m, 1H), 3.76- 3.60 (m, 3H), 3.63-3.42 (m, 4H), 3.06-2.83 (m, 6H), 2.81-2.64 (m, 1H), 2.46 (s, 3H), 2.37-2.26 (m, 6H), 2.18-2.06 (m, 1H), 1.99-1.66 (m, 3H), 1.58 (m, 2H), 1.41 (br s, 1H), 1.19 (d, J = 6.7 Hz, 3H). | Intermediate B2 was used; After Step 1, the sample was purified via SFC using a CHIRALPAK IC (150 × 50) mm, 5 μm, column with a mobile phase of Liquid CO₂: ACN:IPA(1:1)] (50:50)] using a flowrate of 200 mL/min (1st eluting isomer) |
| 1-044 | 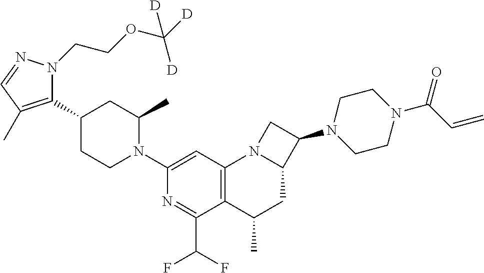 [SMILES: [2H]C([2H])([2H])OCCn1ncc(C)c1[C@H]1CCN(c2cc3c(c(C(F)F)n2)[C@@H](C)C[C@H]2[C@H](N4CCN(C(=O)C=C)CC4)CN32)[C@H](C)C1] 1-(4-((5S,6aS,7R)-4- (difluoromethyl)-2-((2R,4S)-4-(1-(2- (methoxy-d3)ethyl)-4-methyl-1H- pyrazol-5-yl)-2-methylpiperidin-1- yl)-5-methyl-6,6a,7,8-tetrahydro-5H- azeto[1,2-a][1,6]naphthyridin-7- yl)piperazin-1-yl)prop-2-en-1-one | m/z (ESI): 601.4 (M + H)⁺. ¹H NMR (400 MHz, DMSO-d₆) δ (ppm) 7.12 (s, 1H), 6.97-6.67 (m, 2H), 6.12 (dd, J = 16.7, 2.4 Hz, 1H), 5.72-5.65 (m, 2H), 4.60 (br s, 1H), 4.34-4.01 (m, 5H), 3.91 (t, J = 8.4 Hz, 1H), 3.66-3.54 (m, 6H), 3.28 (s, 1H), 3.04-2.77 (m, 3H), 2.36 (d, J = 6.3 Hz, 4H), 2.08-1.94 (m, 5H), 1.83-1.72 (m, 2H), 1.63 (d, J = 12.8 Hz, 1H), 1.42 (q, J = 11.9 Hz, 1H), 1.31 (d, J = 6.4 Hz, 3H), 1.15 (d, J = 6.7 Hz, 3H). | Intermediates E11 and B22 were used |
| 1-047 | 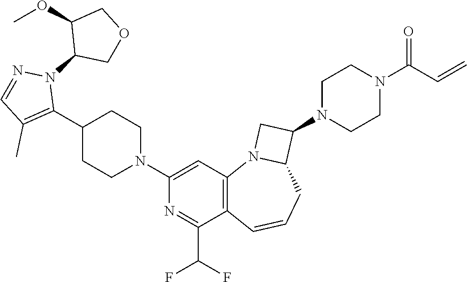 [SMILES: C=CC(=O)N1CCN([C@@H]2CN3c4cc(N5CCC(c6c(C)cnn6[C@@H]6COC[C@@H]6OC)CC5)nc(C(F)F)c4C=CC[C@@H]23)CC1] 1-(4-((7aS,8R)-4-(difluoromethyl)-2- (4-(1-((3R,4R)-4-methoxytetrahydro- 3-furanyl)-4-methyl-1H-pyrazol-5- yl)-1-piperidinyl)-5,6,7,7a,8,9- hexahydroazeto[1,2-a]pyrido[3,4- f]azepin-8-yl)-1-piperazinyl)-2- propen-1-one | m/z (ESI): 626.3 (M + H)⁺. ¹H NMR (DMSO-d₆, 400 MHz): δ (ppm) 7.13 (s, 1H), 6.84-6.77 (m, 1H), 6.71 (t, J = 54.8 Hz, 1H), 6.11 (dd, J = 16.7, 2.4 Hz, 1H), 5.94 (s, 1H), 5.68 (dd, J = 10.4, 2.4 Hz, 1H), 5.02 (q, J = 6.9 Hz, 1H), 4.51- 4.35 (m, 3H), 4.15-3.98 (m, 3H), 3.79 (dd, J = 8.5, 3.7 Hz, 1H), 3.71 (p, J = 7.8 Hz, 3H), 3.60-3.56 (m, 4H), 3.20-3.08 (m, 1H), 3.05-3.03 (m, 1H), 2,98 (s, 3H), 2.92-2.75 (m, 3H), 2.40-2.20 (m, 5H), 2.00 (s, 3H), 1.95-1.68 (m, 7H), 1.40 (d, J = 11.8 Hz, 1H). | B27/B28 intermediate mixture was used |
| 1-048 | 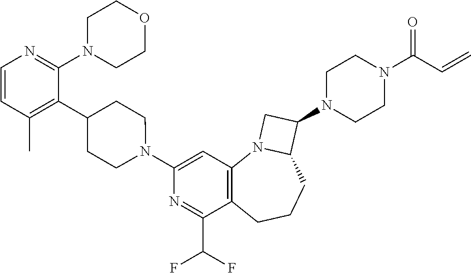 [SMILES: C=CC(=O)N1CCN([C@@H]2CN3c4cc(N5CCC(c6c(C)ccnc6N6CCOCC6)CC5)nc(C(F)F)c4CCC[C@@H]23)CC1] 1-(4-((7aS,8R)-4-(difluoromethyl)-2- (4-(4-methyl-2-(4-morpholinyl)-3- pyridinyl)-1-piperidinyl)-5,6,7,7a,8,9- hexahydroazeto[1,2-a]pyrido[3,4- f]azepin-8-yl)-1-piperazinyl)-2- propen-1-one | m/z (ESI): 622.3 (M + H)⁺. ¹H NMR (400 MHz, DMSO-d₆) δ (ppm) 8.10 (d, J = 4.9 Hz, 1H), 6.98 (d, J = 4.9 Hz, 1H), 6.88-6.61 (m, 2H), 6.11 (dd, J = 16.7, 2.4 Hz, 1H), 5.97 (s, 1H), 5.68 (dd, J = 10.4, 2.4 Hz, 1H), 4.43 (t, J = 11.9 Hz, 2H), 3.73 (m, 3H), 3.51 (m, 8H), 3.02 (t, J = 11.9 Hz, 1H), 2.95-2.83 (m, 3H), 2.77 (br s, 4H), 2.33 (d, J = 7.9 Hz, 3H), 2.24 (d, J = 12.8 Hz, 7H), 1.84 (m, 3H), 1.50 (d, J = 12.5 Hz, 2H), 1.42 (d, J = 10.8 Hz, 1H). One proton overlaps with water peak. | Intermediate B15 was used |
| 1-049 | 1-(4-((7aS,8R)-4-(difluoromethyl)-2- ((3S,4R)-4-(1-(2-methoxyethyl)-4- methyl-1H-pyrazol-5-yl)-3-methyl-1- piperidinyl)-5,6,7,7a,8,9- hexahydroazeto[1,2-a]pyrido[3,4- f]azepin-8-yl)-1-piperazinyl)-2- propen-1-one | m/z (ESI): 598.3 (M + H)⁺. ¹H NMR (500 MHz, MeOH- d4) δ (ppm) 7.22 (s, 1 H), 6.75 (dd, J = 16.9, 10.6 Hz, 1 H), 6.38-6.62 (m, 1 H), 6.20 (dd, J = 16.8, 1.9 Hz, 1 H), 5.94 (s, 1 H), 5.72- 5.78 (m, 1 H), 4.36-4.45 (m, 2 H), 4.28-4.35 (m, 1 H), 4.18-4.25 (m, 1 H), 3.81 (t, J = 7.4 Hz, 1 H), 3.65- 3.76 (m, 8 H), 3.28 (s, 3 H), 3.13-3.22 (m, 1 H), 2.97 (q, J = 6.7 Hz, 1 H), 2.84 (td, J = 12.7, 2.7 Hz, 1 H), 2.74 (td, J = 11.7, 3.8 Hz, 1 H), 2.41-2.54 (m, 6 H), 2.13-2.23 (m, 1 H), 2.02 (s, 3 H), 1.90-2.01 (m, 3 H), 1.80 (br d, J = 3.2 Hz, 2 H), 1.41-1.51 (m, 1 H), 0.79 (d, J = 6.5 Hz, 3 H) | Intermediate B41 was used |
| 1-050 | 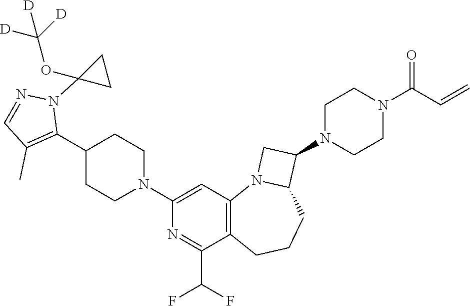 [SMILES: [2H]C([2H])([2H])OC1(n2ncc(C)c2C2CCN(c3cc4c(c(C(F)F)n3)CCC[C@H]3[C@H](N5CCN(C(=O)C=C)CC5)CN43)CC2)CC1] 1-(4-((7aS,8R)-4-(difluoromethyl)-2- (4-(1-(1-(methoxy-d3)cyclopropyl)-4- methyl-1H-pyrazol-5-yl)piperidin-1- yl)-5,6,7,7a,8,9-hexahydroazeto[1,2- a]pyrido[3,4-f]azepin-8-yl)piperazin- 1-yl)prop-2-en-1-one | m/z (ESI): 599.0 (M + H)⁺. 1H NMR (CDCl₃, 400 MHz) δ (ppm) 7.20 (s, 1 H), 6.49-6.67 (m, 2 H), 6.25-6.34 (m, 1 H), 5.79 (s, 1 H), 5.67-5.74 (m, 1 H), 4.33-4.47 (m, 2 H), 3.55-3.79 (m, 7 H), 3.34- 3.46 (m, 1 H), 3.16-3.26 (m, 1 H), 2.91-2.99 (m, 1 H), 2.75-2.89 (m, 2 H), 2.31-2.49 (m, 5 H), 2.06- 2.09 (m, 3 H), 1.89-2.06 (m, 4 H), 1.78-1.88 (m, 3 H), 1.39-1.50 (m, 1 H), 1.36 (br d, J = 2.1 Hz, 4 H) ¹⁹F NMR (CDCl₃, 377 MHz) δ (ppm) −118.5- −103.9 (m, 2F) | Intermediate B7 was used |
| 1-051 | 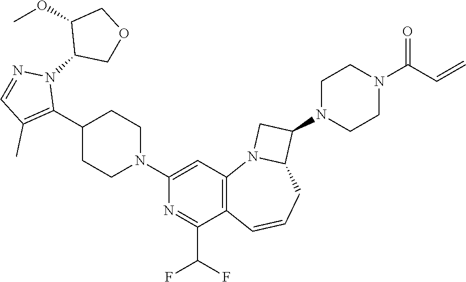 [SMILES: C=CC(=O)N1CCN([C@@H]2CN3c4cc(N5CCC(c6c(C)cnn6[C@H]6COC[C@H]6OC)CC5)nc(C(F)F)c4C=CC[C@@H]23)CC1] 1-(4-((7aS,8R)-4-(difluoromethyl)-2- (4-(1-((3S,4S)-4-methoxytetrahydro- 3-furanyl)-4-methyl-1H-pyrazol-5- yl)-1-piperidinyl)-5,6,7,7a,8,9- hexahydroazeto[1,2-a]pyrido[3,4- f]azepin-8-yl)-1-piperazinyl)-2- propen-1-one | m/z (ESI): 626.3 (M + H)⁺. ¹H NMR (400 MHz, DMSO-d₆) δ (ppm) 7.14 (s, 1H), 6.85-6.55 (m, 2H), 6.11 (dd, J = 16.7, 2.4 Hz, 1H), 5.94 (s, 1H), 5.68 (dd, J = 10.4, 2.4 Hz, 1H), 5.02 (q, J = 6.9 Hz, 1H), 4.47- 4.37 (m, 3H), 4.10 (dd, J = 8.6, 7.3 Hz, 3H), 4.10- 3.98 (m, 4H), 3.78-3.65 (m, 4H), 3.19-3.11 (m, 1H), 3.05-2.96 (m, 4H), 2.93-2.76 (m, 3H), 2.33 (br s, 5H), 2.00 (s, 3H), 1.95-1.80 (m, 7H), 1.40 (d, J = 11.7 Hz, 1H). | B27/B28 intermediate mixture was used |
| 1-052 | 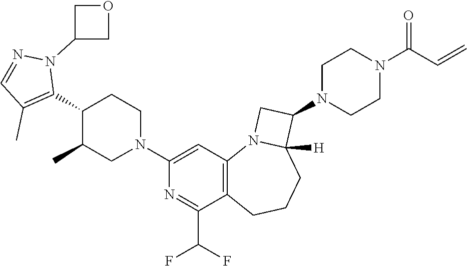 [SMILES: C=CC(=O)N1CCN([C@@H]2CN3c4cc(N5CC[C@@H](c6c(C)cnn6C6COC6)[C@H](C)C5)nc(C(F)F)c4CCC[C@@H]23)CC1] 1-(4-((7aS,8R)-4-(difluoromethyl)-2- ((3S,4R)-3-methyl-4-(4-methyl-1-(3- oxetanyl)-1H-pyrazol-5-yl)-1- piperidinyl)-5,6,7,7a,8,9- hexahydroazeto[1,2-a]pyrido[3,4- f]azepin-8-yl)-1-piperazinyl)-2- propen-1-one | m/z (ESI): 596.35 (M + H)⁺. ¹H NMR (400 MHz, MeOH- d4) δ (ppm) 7.39 (s, 1 H), 6.78 (dd, J = 16.9, 10.7 Hz, 1 H), 6.35-6.69 (m, 1 H), 6.22 (dd, J = 16.8, 2.0 Hz, 1 H), 5.96 (s, 1 H), 5.77 (dd, J = 10.7, 1.9 Hz, 1 H), 5.69- 5.75 (m, 1 H), 5.09-5.16 (m, 1 H), 5.03-5.08 (m, 1 H), 4.96-5.02 (m, 2 H), 4.41 (br d, J = 12.3 Hz, 2 H), 3.80-3.86 (m, 1 H), 3.66- 3.78 (m, 6 H), 3.14-3.25 (m, 1 H), 2.99 (q, J = 6.5 Hz, 1 H), 2.88 (td, J = 12.5, 2.6 Hz, 1 H), 2.59-2.69 (m, 1 H), 2.42-2.57 (m, 6 H), 2.08-2.16 (m, 1 H), 2.05 (s, 3 H), 1.80-2.02 (m, 4 H), 1.66-1.73 (m, 1 H), 1.43-1.55 (m, 1 H), 0.72 (d, J = 6.5 Hz, 3 H) | Intermediate B24 was used |
| 1-053 | 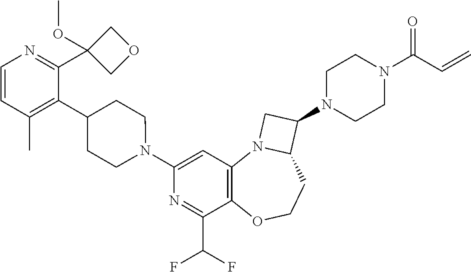 [SMILES: C=CC(=O)N1CCN([C@@H]2CN3c4cc(N5CCC(c6c(C)ccnc6C6(OC)COC6)CC5)nc(C(F)F)c4OCC[C@@H]23)CC1] 1-(4-((7aS,8R)-4-(difluoromethyl)-2- (4-(2-(3-methoxy-3-oxetanyl)-4- methyl-3-pyridinyl)-1-piperidinyl)- 7,7a,8,9-tetrahydro-6H-azeto[1,2- d]pyrido[3,4-b][1,4]oxazepin-8-yl)-1- piperazinyl)-2-propen-1-one | m/z (ESI): 625.3 (M + H)⁺. ¹H NMR (CDCl₃, 400 MHz) δ (ppm) 8.18-8.41 (m, 1 H), 7.03 (d, J = 5.0 Hz, 1 H), 6.49-6.83 (m, 2 H), 6.23-6.37 (m, 1 H), 5.68- 5.78 (m, 2 H), 5.27 (d, J = 6.8 Hz, 2 H), 4.79-4.95 (m, 2 H), 4.30-4.47 (m, 3 H), 4.15-4.23 (m, 1 H), 3.88-3.99 (m, 1 H), 3.80- 3.87 (m, 1 H), 3.55-3.79 (m, 5 H), 3.06-3.12 (m, 1 H), 3.06 (s, 3 H), 2.67- 2.85 (m, 3 H), 2.45-2.47 (m, 3 H), 2.38-2.45 (m, 4 H), 2.16-2.31 (m, 3 H), 2.06-2.14 (m, 1 H), 1.60- 1.64 (m. 2 H) ¹⁹F NMR (CDCl₃, 376 MHz) δ (ppm) −117.69 (s, 2F) | Intermediates E3 and B1 were used |
| 1-055 | 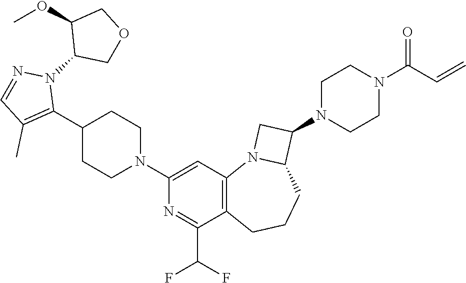 [SMILES: C=CC(=O)N1CCN([C@@H]2CN3c4cc(N5CCC(c6c(C)cnn6[C@H]6COC[C@@H]6OC)CC5)nc(C(F)F)c4CCC[C@@H]23)CC1] 1-(4-((7aS,8R)-4-(difluoromethyl)-2- (4-(1-((3S,4R)-4-methoxytetrahydro- 3-furanyl)-4-methyl-1H-pyrazol-5- yl)-1-piperidinyl)-5,6,7,7a,8,9- hexahydroazeto[1,2-a]pyrido[3,4- f]azepin-8-yl)-1-piperazinyl)-2- propen-1-one | m/z (ESI): 624.3 (M + H)⁺. ¹H NMR (400 MHz, DMSO-d₆) δ (ppm) 7.21 (s, 1H), 6.87-6.55 (m, 2H), 6.11 (dd, J = 16.7, 2.4 Hz, 1H), 5.94 (s, 1H), 5.68 (dd, J = 10.4, 2.4 Hz, 1H), 4.95 (ddd, J = 7.6, 5.5, 2.4 Hz, 1H), 4.42 (br s, 2H), 4.25- 4.16 (m, 2H), 4.00 (dd, J = 9.7, 4.8 Hz, 1H), 3.84 (dd, J = 9.7, 2.4 Hz, 1H), 3.78- 3.67 (m, 4H), 3.63-3.48 (m, 4H), 3.27 (s, 3H), 3.21- 3.11 (m, 1H), 3.07-2.97 (m, 1H), 2.90 (q, J = 6.6 Hz, 1H), 2.79 (d, J = 16.4 Hz, 2H), 2.41-2.27 (m, 5H), 2.00 (s, 3H), 1.94- 1.71 (m, 7H), 1.40 (d, J = 10.8 Hz, 1H). | B29/B30 intermediate mixture was used |
| 1-056 | 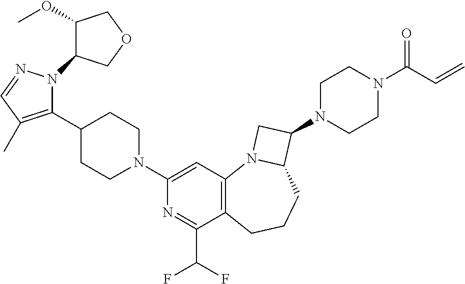 [SMILES: C=CC(=O)N1CCN([C@@H]2CN3c4cc(N5CCC(c6c(C)cnn6[C@@H]6COC[C@H]6OC)CC5)nc(C(F)F)c4CCC[C@@H]23)CC1] 1-(4-((7aS,8R)-4-(difluoromethyl)-2- (4-(1-((3R,4S)-4-methoxytetrahydro- 3-furanyl)-4-methyl-1H-pyrazol-5- yl)-1-piperidinyl)-5,6,7,7a,8,9- hexahydroazeto[1,2-a]pyrido[3,4- f]azepin-8-yl)-1-piperazinyl)-2- propen-1-one | m/z (ESI): 626.3 (M + H)⁺. ¹H NMR (400 MHz, DMSO-d₆) δ (ppm) 7.21 (s, 1H), 6.86-6.54 (m, 2H), 6.11 (dd, J = 16.7, 2.4 Hz, 1H), 5.94 (s, 1H), 5.68 (dd, J = 10.4, 2.4 Hz, 1H), 4.95 (ddd, J = 7.5, 5.4, 2.3 Hz, 1H), 4.42 (s, 2H), 4.25- 4.17 (m, 2H), 4.00 (dd, J = 9.7, 4.9 Hz, 1H), 3.84 (dd, J = 9.6, 2.4 Hz, 1H), 3.80- 3.67 (m, 4H), 3,55 (d, J = 12.2 Hz, 4H), 3.27 (s, 3H), 3.14 (d, J = 12.2 Hz, 1H), 3.02 (t, J = 11.9 Hz, 1H), 2.90 (q, J = 6.6 Hz, 1H), 2.81 (t, J = 12.3 Hz, 2H), 2.37 (br s, 5H), 2.00 (s, 3H), 1.1.95-1.77 (m, 7H), 1.40 (d, J = 11.8 Hz, 1H) | B29/B30 intermediate mixture was used |
| 1-057 | 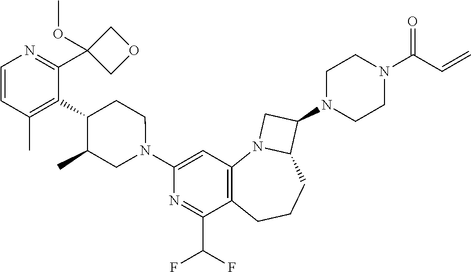 [SMILES: C=CC(=O)N1CCN([C@@H]2CN3c4cc(N5CC[C@@H](c6c(C)ccnc6C6(OC)COC6)[C@H](C)C5)nc(C(F)F)c4CCC[C@@H]23)CC1] 1-(4-((7aS,8R)-4-(difluoromethyl)-2- ((3S,4R)-4-(2-(3-methoxy-3- oxetanyl)-4-methyl-3-pyridinyl)-3- methyl-1-piperidinyl)-5,6,7,7a,8,9- hexahydroazeto[1,2-a]pyrido[3,4- f]azepin-8-yl)-1-piperazinyl)-2- propen-1-one | m/z (ESI): 637.2 (M + H)⁺. ¹H NMR (400 MHz, DMSO-d₆) δ (ppm) 8.29 (d, J = 4.9 Hz, 1H), 7.18 (d, J = 5.0 Hz, 1H), 6.86-6.54 (m, 2H), 6.11 (dd, J = 16.6, 2.4 Hz, 1H), 5.94 (s, 1H), 5.68 (dd, J = 10.5, 2.4 Hz, 1H), 5.14 (dd, J = 13.8, 7.2 Hz, 2H), 4.77 (t, J = 7.4 Hz, 2H), 4.47-4.32 (m, 2H), 3.79-3.70 (m, 3H), 3.62- 3.48 (m, 4H), 3.09-2.97 (m, 1H), 2.95 (s, 3H), 2.92- 2.84 (m, 1H), 2.70 (t, J = 12.0 Hz, 1H), 2.39 (s, 3H), 2.38-2.21 (m, 8H), 2.07- 1.69 (m, 4H), 1.65 (d, J = 12.9 Hz, 1H), 1.41 (s, 1H), 0.67 (d, J = 5.0 Hz, 3H). | Intermediate B39 was used |
| 1-058 | 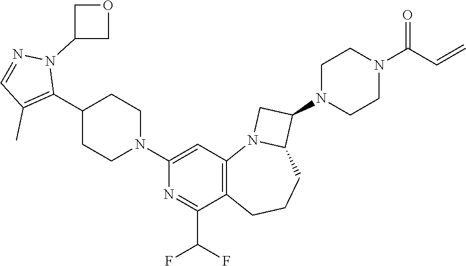 [SMILES: C=CC(=O)N1CCN([C@@H]2CN3c4cc(N5CCC(c6c(C)cnn6C6COC6)CC5)nc(C(F)F)c4CCC[C@@H]23)CC1] 1-(4-((7aS,8R)-4-(difluoromethyl)-2- (4-(4-methyl-1-(3-oxetanyl)-1H- pyrazol-5-yl)-1-piperidinyl)- 5,6,7,7a,8,9-hexahydroazeto[1,2- a]pyrido[3,4-f]azepin-8-yl)-1- piperazinyl)-2-propen-1-one | m/z (ESI): 582.25 (M + H)⁺. ¹H NMR (500 MHz, MeOH- d4) δ (ppm) 7.32 (s, 1 H), 6.75 (dd, J = 16.7, 10.6 Hz, 1 H), 6.37-6.62 (m, 1 H), 6.20 (dd, J = 16.9, 1.9 Hz, 1 H), 5.93 (s, 1 H), 5.72- 5.77 (m, 1 H), 5.65-5.72 (m, 1 H), 5.06 (t, J = 6.4 Hz, 2 H), 4.96-5.01 (m, 2 H), 4.37-4.47 (m, 2 H), 3.77- 3.83 (m, 1 H), 3.65-3.75 (m, 6 H), 3.13-3.22 (m, 1 H), 2.93-3.02 (m, 2 H), 2.82-2.90 (m, 2 H), 2.45 (br s, 5 H), 2.06 (s, 3 H), 1.90-2.02 (m, 5 H), 1.80- 1.89 (m, 1 H), 1.71 (br d, J = 13.0 Hz, 2 H) | Intermediate B23 was used |
| 1-059 | 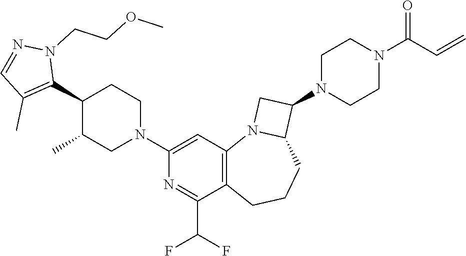 [SMILES: C=CC(=O)N1CCN([C@@H]2CN3c4cc(N5CC[C@H](c6c(C)cnn6CCOC)[C@@H](C)C5)nc(C(F)F)c4CCC[C@@H]23)CC1] 1-(4-((7aS,8R)-4-(difluoromethyl)-2- ((3R,4S)-4-(1-(2-methoxyethyl)-4- methyl-1H-pyrazol-5-yl)-3-methyl-1- piperidinyl)-5,6,7,7a,8,9- hexahydroazeto[1,2-a]pyrido[3,4- f]azepin-8-yl)-1-piperazinyl)-2- propen-1-one | m/z (ESI): 598.3 (M + H)⁺. ¹H NMR(DMSO-d₆, 400 MHz): δ (ppm) 7.16 (s, 1H), 6.-6.55 (m, 2H), 6.11 (dd, J = 16.7, 2.4 Hz, 1H), 5.95 (s, 1H), 5.68 (dd, J = 10.4, 2.4 Hz, 1H), 4.40 (t, J = 14.8 Hz, 2H), 4.39-4.13 (m, 2H), 3.72-3.80 (m, 3H), 3.62 (t, J = 5.5 Hz, 2H), 3.56 (br s, 4H), 3.21 (s, 3H), 3.02 (t, J = 11.7 Hz, 1H), 2.90 (q, J = 6.5 Hz, 1H), 2.81-2.64 (m, 2H), 2.43-2.33 (m, 6H), 2.05- 1.65 (m, 6H), 1.96 (s, 3H), 1.41 (br s, 1H), 0.73 (d, J = 6.5 Hz, 3H) | Intermediate B42 was used |
| 1-061 | 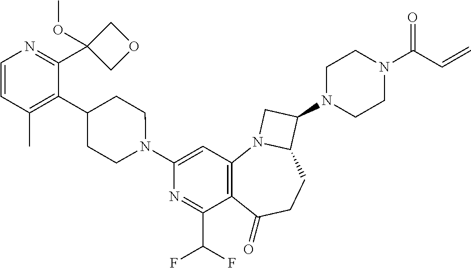 [SMILES: C=CC(=O)N1CCN([C@@H]2CN3c4cc(N5CCC(c6c(C)ccnc6C6(OC)COC6)CC5)nc(C(F)F)c4C(=O)CC[C@@H]23)CC1] (7aS,8R)-4-(difluoromethyl)-2-(4-(2- (3-methoxy-3-oxetanyl)-4-methyl-3- pyridinyl)-1-piperidinyl)-8-(4-(2- propenoyl)-1-piperazinyl)-7,7a,8,9- tetrahydroazeto[1,2-a]pyrido[3,4- f]azepin-5(6H)-one | m/z (ESI): 637.0 (M + H)⁺. ¹H NMR (400 MHz, CDCl₃) δ (ppm) 8.31 (d, J = 4.8 Hz, 1 H), 7.04 (d, J = 5.0 Hz, 1 H), 7.06 (br t, J = 54.8 Hz, 1 H), 6.55 (dd, J = 16.8, 10.6 Hz, 1 H), 6.31 (dd, J = 16.9, 1.9 Hz, 1 H), 5.70-5.75 (m, 1 H), 5.65 (s, 1 H), 5.29 (d, J = 6.9 Hz, 2 H), 4.93 (d, J = 7.3 Hz, 2 H), 4.62 (br t, J = 13.8 Hz, 2 H), 4.09 (t, J = 6.9 Hz, 1 H), 3.97 (dt, J = 11.3, 5.9 Hz, 1 H), 3.80 (br d, J = 1.9 Hz, 1 H), 3.43-3.68 (m, 4 H), 3.08 (s, 3 H), 3.04-3.14 (m, 1 H), 2.79-2.99 (m, 3 H), 2.60 (ddd, J = 11.0, 6.3, 2.4 Hz, 1 H), 2.45 (s, 3 H), 2.12-2.47 (m, 9 H), 1.70 (br d, J = 11.5 Hz, 2 H) ¹⁹F NMR (376 MHz, CDCl₃) δ (ppm) −120.96- −116.65 (m, 2 F); | Intermediates E5 and B1 were used; Step 4. Acrylic anhydride was used |
| 1-062 | 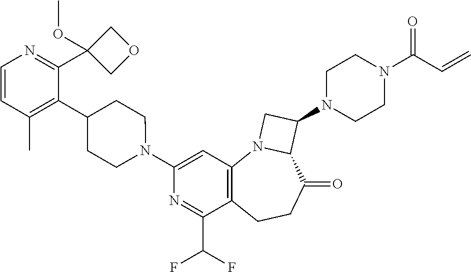 [SMILES: C=CC(=O)N1CCN([C@@H]2CN3c4cc(N5CCC(c6c(C)ccnc6C6(OC)COC6)CC5)nc(C(F)F)c4CCC(=O)[C@@H]23)CC1] (7aR,8R)-4-(difluoromethyl)-2-(4-(2- (3-methoxy-3-oxetanyl)-4-methyl-3- pyridinyl)-1-piperidinyl)-8-(4-(2- propenoyl)-1-piperazinyl)-5,6,8,9- tetrahydroazeto[1,2-a]pyrido[3,4- f]azepin-7(7aH)-one | m/z (ESI): 637.0 (M + H)⁺; ¹H NMR (500 MHz, CDCl₃) δ (ppm) 8.31 (d, J = 4.9 Hz, 1 H), 7.05 (d, J = 4.9 Hz, 1 H), 6.35-6.62 (m, 2 H), 6.32 (dd, J = 16.9, 1.8 Hz, 1 H), 5.74 (dd, J = 10.5, 1.8 Hz, 1 H), 5.62 (s, 1 H), 5.29 (d, J = 6.9 Hz, 2 H), 5.03 (d, J = 5.4 Hz, 1 H), 4.93 (d, J = 7.4 Hz, 2 H), 4.34-4.44 (m, 2 H), 3.85- 3.94 (m, 2 H), 3.81; (t, J = 6.7 Hz, 1 H), 3.50-3.72 (m, 4 H), 3.31-3.38 (m, 1 H), 3.08 (s, 3 H), 2.91- 3.05 (m, 2 H), 2.73-2.86 (m, 4 H), 2.40-2.64 (m, 7 H), 2.21 (qt, J = 12.5, 3.9 Hz, 2 H), 1.66 (br d, J = 12.3 Hz, 2 H); ¹⁹F NMR (471 MHz, CDCl₃) δ (ppm) −112.67- −109.37 (m, 2 F); | Intermediates E4 and B1 were used; Step 4. Acrylic anhydride was used |
| 1-064 | 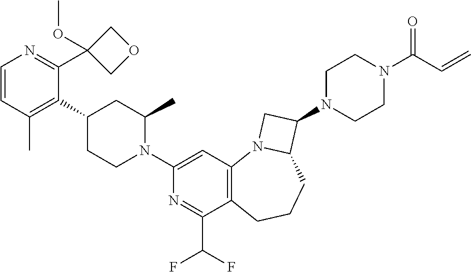 [SMILES: C=CC(=O)N1CCN([C@@H]2CN3c4cc(N5CC[C@H](c6c(C)ccnc6C6(OC)COC6)C[C@H]5C)nc(C(F)F)c4CCC[C@@H]23)CC1] 1-(4-((7aS,8R)-4-(difluoromethyl)-2- ((2R,4S)-4-(2-(3-methoxy-3- oxetanyl)-4-methyl-3-pyridinyl)-2- methyl-1-piperidinyl)-5,6,7,7a,8,9- hexahydroazeto[1,2-a]pyrido[3,4- f]azepin-8-yl)-1-piperazinyl)-2- propen-1-one | m/z (ESI): 637.4 (M + H)⁺. ¹H NMR (400 MHz, DMSO-d₆) δ (ppm) 8.29 (d, J = 4.9 Hz, 1H), 7.18 (d, J = 5.0 Hz, 1H), 6.54-6.90 (m, 2H), 6.11 (dd, J = 16.7, 2.4 Hz, 1H), 5.87 (s, 1H), 5.68 (dd, J = 10.4, 2.4 Hz, 1H), 5.18 (dd, J = 11.9, 7.0 Hz, 2H), 4.71-4.88 (m, 3H), 4.29 (d, J = 13.2 Hz, 1H), 3.63-3.77 (m, 3H), 3.56 (d, J = 12.3 Hz, 4H), 2.97-2.81 (m, 7H), 2.44 (s, 3H), 2.25-2.40 (m, 6H), 1.97-2.14 (m, 1H), 1.67-1.97 (m, 4H), 1.62 (d, J = 12.6 Hz, 1H), 1.50 (d, J = 12.7 Hz, 1H), 1.41 (br s, 1H), 1.14 (d, J = 6.8 Hz, 3H). | Intermediate B10 was used; |
| 1-065 | 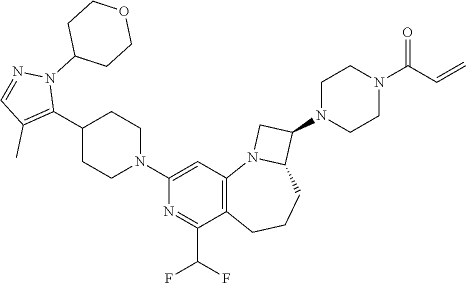 [SMILES: C=CC(=O)N1CCN([C@@H]2CN3c4cc(N5CCC(c6c(C)cnn6C6CCOCC6)CC5)nc(C(F)F)c4CCC[C@@H]23)CC1] 1-(4-((7aS,8R)-4-(difluoromethyl)-2- (4-(4-methyl-1-(tetrahydro-2H-pyran- 4-yl)-1H-pyrazol-5-yl)-1-piperidinyl)- 5,6,7,7a,8,9-hexahydroazeto[1,2- a]pyrido[3,4-f]azepin-8-yl)-1- piperazinyl)-2-propen-1-one | m/z (ESI): 610.3 (M + H)⁺. ¹H NMR (400 MHz, DMSO-d₆) δ (ppm) 7.16 (s, 1H), 6.86-6.56 (m, 2H), 6.11 (dd, J = 16,7, 2.4 Hz, 1H), 5.94 (s, 1H), 5.68 (dd, J = 10.5, 2.4 Hz, 1H), 4.47- 4.36 (m. 3H), 3.94 (dd, J = 11.5, 4.3 Hz, 2H), 3.65- 3.79 (m, 3H), 3.61-3.51 (m, 4H), 3.45 (t, J = 11.9 Hz, 2H), 3.17-3.02 (m, 2H), 2.93-2.79 (m, 3H), 2.40-2.27 (m, 5H), 2.11- 2.01 (m, 2H), 1.99 (s, 3H), 1.95-1.65 (m, 9H), 1.41 (d, J = 11.5 Hz, 1H). | Intermediate B32 was used; |
| 1-066 | 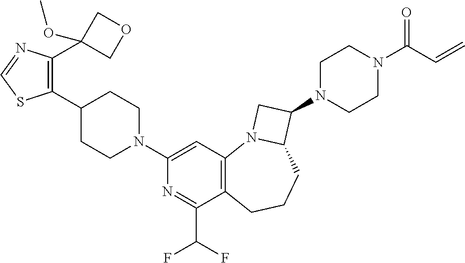 [SMILES: C=CC(=O)N1CCN([C@@H]2CN3c4cc(N5CCC(c6scnc6C6(OC)COC6)CC5)nc(C(F)F)c4CCC[C@@H]23)CC1] 1-(4-((7aS,8R)-4-(difluoromethyl)-2- (4-(4-(3-methoxy-3-oxetanyl)-1,3- thiazol-5-yl)-1-piperidinyl)- 5,6,7,7a,8,9-hexahydroazeto[1,2- a]pyrido[3,4-f]azepin-8-yl)-1- piperazinyl)-2-propen-1-one | m/z (ESI): 615.0 (M + H)⁺. ¹H NMR (400 MHz, MeOH-d₄) δ (ppm) 8.82 (s, 1 H), 6.74 (dd, J = 16.9, 10.7 Hz, 1 H), 6.33-6.64 (m, 1 H), 6.20 (dd, J = 16.7, 1.9 Hz, 1 H), 5.92 (s, 1 H), 5.74 (dd, J = 10.7, 1.9 Hz, 1 H), 5.14 (d, J = 7.1 Hz, 2 H), 4.93 (d, J = 6.7 Hz, 2 H), 4.35-4.49 (m, 2 H), 3.77- 3.83 (m, 1 H), 3.65-3.76 (m, 6 H), 3.13-3.21 (m, 1 H), 3.08 (s, 3 H), 2.94- 3.04 (m, 2 H), 2.85 (br t, J = 11.9 Hz, 2 H), 2.46 (br d, J = 4.6 Hz, 5 H), 1.93-2.05 (m, 4 H), 1.79-1.89 (m, 1 H), 1.59-1.71 (m, 2 H), 1.41-1.54 (m, 1 H)/ | Intermediate B38 was used; |
| 1-102 | 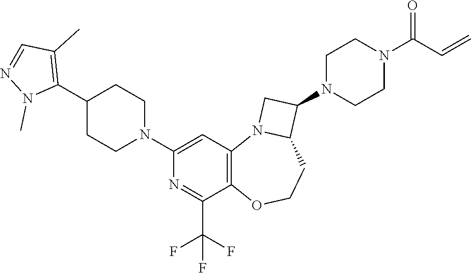 [SMILES: C=CC(=O)N1CCN([C@@H]2CN3c4cc(N5CCC(c6c(C)cnn6C)CC5)nc(C(F)(F)F)c4OCC[C@@H]23)CC1] 1-(4-((7aS,8R)-2-(4-(1,4-dimethyl- 1H-pyrazol-5-yl)-1-piperidinyl)-4- (trifluoromethyl)-7,7a,8,9-tetrahydro- 6H-azeto[1,2- | m/z (ESI): 560.2 (M + H)⁺; ¹H NMR (400 MHz, CDCl₃) δ ppm 7.11-7.23 (m, 1 H), 6.47-6.63 (m, 1 H), 6.22-6.37 (m, 1 H), 5.75-5.79 (m, 1 H), 5.69- 5.74 (m, 1 H), 4.28-4.46 (m, 3 H), 4.13-4.24 (m, 1 H), 3.87-3.96 (m, 1 H), 3.84 (s, 3 H), 3.51-3.96 (m, 5 H), 3.02-3.15 (m, 1 H), 2.71-2.96 (m, 3 H), 2.37-2.49 (m, 4 H), 2.18- 2.33 (m, 1 H), 2.1.94-2.14 (m, 3 H), 2.07 (s, 3 H), 1.77- 1.88 (m, 2 H); ¹⁹F NMR (376 MHz, CDCl₃) δ ppm −65.57- −65.07 (m, 3 F). | Intermediates E3 and B20 were used |
| 1-106 | 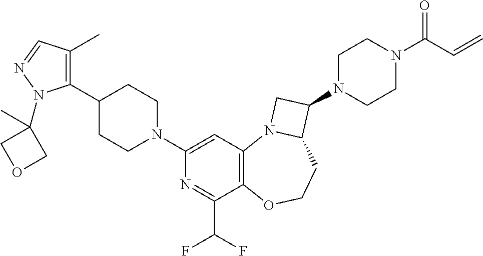 [SMILES: C=CC(=O)N1CCN([C@@H]2CN3c4cc(N5CCC(c6c(C)cnn6C6(C)COC6)CC5)nc(C(F)F)c4OCC[C@@H]23)CC1] 1-(4-((7aS,8R)-4-(difluoromethyl)-2- (4-(4-methyl-1-(3-methyloxetan-3- yl)-1H-pyrazol-5-yl)piperidin-1-yl)- 7,7a,8,9-tetrahydro-6H-azeto[1,2- d]pyrido[3,4-b][1,4]oxazepin-8- yl)piperazin-1-yl)prop-2-en-1-one | m/z (ESI): 598.0 (M + H)⁺; ¹H NMR (400 MHz, CDCl₃) δ ppm 7.23 (s, 1 H), 6.51-6.82 (m, 2 H), 6.31 (dd, J = 16.9, 1.9 Hz, 1 H), 5.70-5.74 (m, 2 H), 5.26 (d, J = 6.1 Hz, 2 H), 4.65 (d, J = 6.5 Hz, 2 H), 4.32-4.42 (m, 3 H), 4.15-4.23 (m, 1 H), 3.90-3.98 (m, 1 H), 3.83-3.83 (m, 1 H), 3.57- 3.78 (m, 5 H), 3.09 (4, J = 6.5 Hz, 1 H), 2.71 (br t, J = 12.1 Hz, 2 H), 2.43 (br t, J = 4.8 Hz, 4 H), 2.09-2.31 (m, 5 H), 2.07 (s, 3 H), 1.83 (s, 3 H), 1.78 (br d, J = 12.8 Hz, 2 H); ¹⁹F NMR (376 MHz, CDCl₃) δ ppm −121.5-−116.7 (m, 2F). | Intermediates E-3′ and B23 were used. |

### Method 3

### Example 67:1-(4-((7aS,8R)-4-(Difluoromethyl)-2-(4-(2-(1-methoxycyclopropyl)-4-methyl-3-pyridinyl)-1-piperidinyl)-5,6,7,7a,8,9-hexahydroazeto[1,2-a]pyrido[3,4-f]azepin-8-yl)-1-piperazinyl)-2-propen-1-one

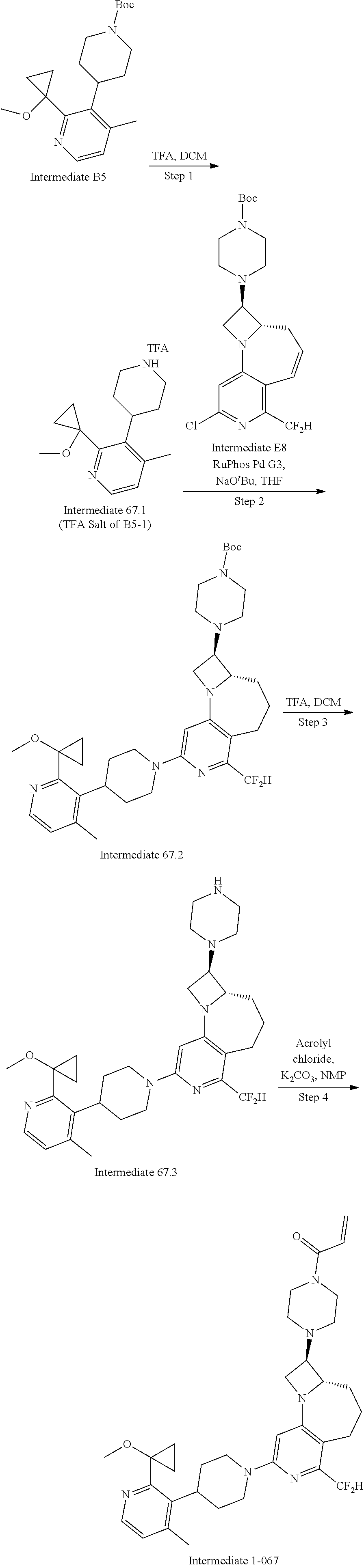

Step 1. 2-(1-Methoxycyclopropyl)-4-methyl-3-(piperidin-4-yl)pyridine trifluoroacetate, Intermediate 67.1. To a solution of Intermediate B5 (250 mg, 0.722 mmol) in DCM (2.0 mL) was added TFA (383 mg, 3.35 mmol), and the reaction mixture was stirred at rt for 30 min. The reaction mixture was concentrated to afford Intermediate 67.1 as a TFA salt, m/z (ESI): 247.0 (M+H)⁺.

Step 2. tert-Butyl 4-((7aS,8R)-4-(difluoromethyl)-2-(4-(2-(1-methoxycyclopropyl)-4-methylpyridin-3-yl)piperidin-1-yl)-5,6,7,7a,8,9-hexahydroazeto[1,2-a]pyrido[3,4-f]azepin-8-yl)piperazine-1-carboxylate, Intermediate 67.2. The crude product from Step 1 was redissolved in THF (7.0 mL), and NaO^{t}Bu (693 mg, 7.22 mmol, Sigma-Aldrich Corporation) was added. The mixture was stirred at rt for 15 min. To the reaction mixture was added a solution of RuPhos Pd G3 (61.4 mg, 0.072 mmol, AA Blocks LLC) and Intermediate E8 (320 mg, 0.722 mmol) in THF (3 mL), and the reaction mixture was heated to 80° C., and stirred for 1 h. The reaction mixture was diluted with sat. aq. NH₄Cl and EtOAc, and the layers were separated. The aqueous layer was extracted with EtOAc and the combined organic extracts were washed with brine, dried over Na₂SO₄, filtered, and concentrated. The crude material was purified by chromatography eluting with a gradient of 0% to 40% EtOAc in heptane, to provide Intermediate 67.2 (0.353 g, 0.541 mmol, 75% yield), m/z (ESI): 653.2 (M+H)⁺.

Step 3. (7aS,8R)-4-(Difluoromethyl)-2-(4-(2-(1-methoxycyclopropyl)-4-methylpyridin-3-yl)piperidin-1-yl)-8-(piperazin-1-yl)-5,6,7,7a,8,9-hexahydroazeto[1,2-a]pyrido[3,4-f]azepine, Intermediate 67.3. To a solution of Intermediate 67.2 (0.353 g, 0.541 mmol) in DCM (3 mL) was added TFA (1 mL, 13.42 mmol) and the reaction mixture was stirred at rt for 30 min. The reaction mixture was concentrated, redissolved in EtOAc, washed with sat. aq. Na₂CO₃, and extracted with EtOAc/EtOH (3:1). The organic extracts were washed with brine, dried over Na₂SO₄, filtered, and concentrated in vacuo. The crude material was purified by chromatography, eluting with a gradient of 0% to 20% MeOH in DCM, to provide Intermediate 67.3 (132 mg, 0.239 mmol, 44% yield), m/z (ESI): 553.0 (M+H)⁺.

Step 4. 1-(4-((7aS,8R)-4-(Difluoromethyl)-2-(4-(2-(1-methoxycyclopropyl)-4-methyl-3-pyridinyl)-1-piperidinyl)-5,6,7,7a,8,9-hexahydroazeto[1,2-a]pyrido[3,4-f]azepin-8-yl)-1-piperazinyl)-2-propon-1-one, Compound 1-067. To a solution of Intermediate 67.3 (98 mg, 0.177 mmol) in DCM (3 mL) was added acryloyl chloride (0.2 M solution in DCM, 0.887 mL, 0.177 mmol, Sigma-Aldrich Corporation), and the reaction mixture was stirred at rt for 30 min. The reaction mixture was concentrated in vacuo. The crude material was purified by chromatography eluting with a gradient of 0% to 80% EtOAc/EtOH (3:1) in heptane to provide 1-(4-((7aS,8R)-4-(difluoromethyl)-2-(4-(2-(1-methoxycyclopropyl)-4-methyl-3-pyridinyl)-1-piperidinyl)-5,6,7,7a,8,9-hexahydroazeto[1,2-a]pyrido[3,4-f]azepin-8-yl)-1-piperazinyl)-2-propen-1-one, Compound 1-067 (46.3 mg, 0.076 mmol, 43% yield), m/z (ESI): 607.0 (M+H)⁺. ¹H NMR (CDCl₃, 400 MHz) δ (ppm) 8.24 (d, J=5.0 Hz, 1H), 6.98 (d, J=5.0 Hz, 1H), 6.35-6.67 (m, 2H), 6.30 (dd, J=16.8, 1.8 Hz, 1H), 5.80 (s, 1H), 5.71 (dd, J=10.6, 1.8 Hz, 1H), 4.37-4.49 (m, 2H), 3.95-4.06 (m, 1H), 3.56-3.80 (m, 7H), 3.18-3.26 (m, 1H), 3.17 (s. 3H), 2.86-2.99 (m, 3H), 2.39-2.49 (m, 8H), 2.16-2.31 (m, 2H), 1.79-2.05 (m, 3H), 1.73 (br d, J=11.9 Hz, 2H), 1.40-1.50 (m, 1H), 1.10-1.20; ¹⁹F NMR (CDCl₃, 377 MHz) δ (ppm)-110.54 (d, 2 F, J=75.4 Hz).

Compounds in Table 2-3 were prepared following the procedure described in Method 3, using appropriate starting materials. All starting materials are commercially available or are described in the Intermediates section above.

TABLE 2-3

| | | LCMS: (ESI + ve ion) m/z; | |
|---|---|---|---|
| Compound | Chemical Structure & Name | NMR | Comments |
| 1-068 | 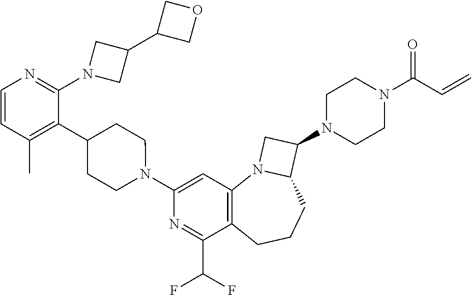 [SMILES: C=CC(=O)N1CCN([C@@H]2CN3c4cc(N5CCC(c6c(C)ccnc6N6CC(C7COC7)C6)CC5)nc(C(F)F)c4CCC[C@@H]23)CC1] 1-(4-((7aS,8R)-4-(difluoromethyl)- 2-(4-(4-methyl-2-(3-(3-oxetanyl)-1- azetidinyl)-3-pyridinyl)-1- piperidinyl)-5,6,7,7a,8,9- hexahydroazeto[1,2-a]pyrido[3,4- f]azepin-8-yl)-1-piperazinyl)-2- propen-1-one | m/z (ESI): 648.0 (M + H)⁺. ¹H NMR (400 MHz, DMSO-d₆) δ (ppm) 7.87 (d, J = 5.0 Hz, 1 H), 6.68-6.86 (m, 2 H), 6.55-6.60 (m, 1 H), 6.10 (dd, J = 16.7, 2.5 Hz, 1 H), 5.94 (s, 1 H), 5.65- 5.71 (m, 1 H), 4.70 (dd, J = 7.7, 6.1 Hz, 2 H), 4.38-4.49 (m, 2 H), 4.31 (t, J = 6.0 Hz, 2 H), 4.09 (td, J = 8.2, 1.1 Hz, 2 H), 3.67- 3.77 (m, 5 H), 3.55 (br d, J = 1.9 Hz, 4 H), 3.17-3.24 (m, 1 H), 2.88-3.09 (m, 4 H), 2.74-2.86 (m, 2 H), 2.27-2.38 (m, 9 H), 2.01-2.16 (m, 2 H), 1.69-1.95 (m, 3 H), 1.60 (br d, J = 11.1 Hz, 2 H) | Intermediate B37 was used Step 4. Acrylic anhydride was used |
| 1-069 | 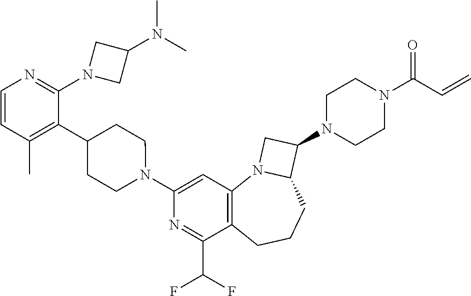 [SMILES: C=CC(=O)N1CCN([C@@H]2CN3c4cc(N5CCC(c6c(C)ccnc6N6CC(N(C)C)C6)CC5)nc(C(F)F)c4CCC[C@@H]23)CC1] 1-(4-((7aS,8R)-4-(difluoromethyl)- 2-(4-(2-(3-(dimethylamino)-1- azetidinyl)-4-methyl-3-pyridinyl)-1- piperidinyl)-5,6,7,7a,8,9- hexahydroazeto[1,2-a]pyrido[3,4- f]azepin-8-yl)-1-piperazinyl)-2- propen-1-one | m/z (ESI): 635.05 (M + H)⁺. ¹H NMR (500 MHz, DMSO-d₆) δ (ppm) 7.86 (d, J = 4.9 Hz, 1 H), 6.69-6.86 (m, 2 H), 6.59 (d, J = 5.1 Hz, 1 H), 6.11 (dd, J = 16.7, 2.3 Hz, 1 H), 5.94 (s, 1 H), 5.63- 5.71 (m, 1 H), 4.38-4.48 (m, 2 H), 3.93-3.99 (m. 2 H), 3.68- 3.79 (m, 5 H), 3.50-3.61 (m, 5 H), 2.96-3.06 (m, 3 H), 2.77- 2.94 (m, 3 H), 2.34 (br dd, J = 4.4, 3.4 Hz, 5 H), 2.28 (s, 3 H), 2.07 (s, 8 H), 1.72-1.95 (m, 3 H), 1.58-1.63 (m, 2 H). | Intermediate B12 was used; Step 4. Acrylic anhydride was used |
| 1-070 | 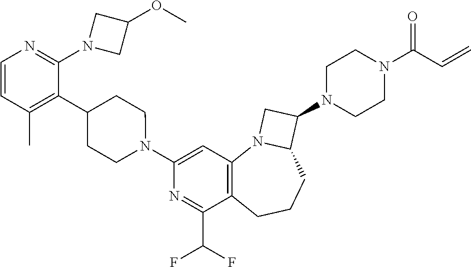 [SMILES: C=CC(=O)N1CCN([C@@H]2CN3c4cc(N5CCC(c6c(C)ccnc6N6CC(OC)C6)CC5)nc(C(F)F)c4CCC[C@@H]23)CC1] 1-(4-((7aS,8R)-4-(difluoromethyl)- 2-(4-(2-(3-methoxy-1-azetidinyl)-4- methyl-3-pyridinyl)-1-piperidinyl)- 5,6,7,7a,8,9-hexahydroazeto[1,2- a]pyrido[3,4-f]azepin-8-yl)-1- piperazinyl)-2-propen-1-one | m/z (ESI): 622.15 (M + H)⁺. ¹H NMR (500 MHz, MeOH-d4) δ (ppm) 7.83 (d, J = 5.2 Hz, 1 H), 6.75 (dd, J = 16.8, 10.7 Hz, 1 H), 6.63 (d, J = 5.2 Hz, 1 H), 6.38- 6.62 (m, 1 H), 6.20 (dd, J = 16.7, 1.9 Hz, 1 H), 5.95 (s, 1 H), 5.75 (dd, J = 10.7, 1.9 Hz, 1 H), 4.41- 4.50 (m, 2 H), 4.25-4.31 (m, 1 H), 4.18-4.23 (m, 2 H), 3.86 (dd, J = 8.4, 4.6 Hz, 2 H), 3.79- 3.83 (m, 1 H), 3.64-3.76 (m, 6 H), 3.33 (s, 3 H), 3.05-3.22 (m, 3 H), 2.97 (q, J = 6.7 Hz, 1 H), 2.85-2.92 (m, 2 H), 2.46 (br s, 5 H), 2.34 (s, 3 H), 2.20-2.29 (m, 2 H), 1.90-2.04 (m, 2 H), 1.80- 1.90 (m, 1 H), 1.61-1.67 (m, 2 H). | Intermediate B14 was used |
| 1-073 | 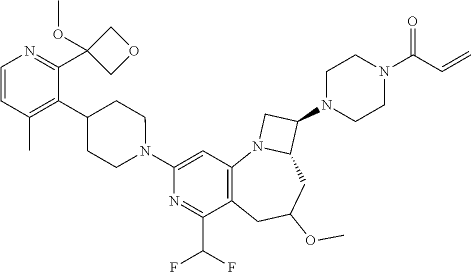 1-(4-((7aS.8R)-4-(difluoromethyl)- 6-methoxy-2-(4-(2-(3- methoxyoxetan-3-yl)-4- methylpyridin-3-yl)piperidin-1-yl)- 5,6,7,7a,8,9-hexahydroazeto[1,2- a]pyrido[3,4-f]azepin-8- yl)piperazin-1-yl)prop-2-en-1-one | m/z (ESI): 653.0 (M + H)⁺; ¹H NMR (400 MHz, CDCl₃) δ (ppm) 8.27-8.33 (m. 1 H), 7.04 (d, J = 5.0 Hz, 1 H), 6.37-6.68 (m, 2 H), 6.29-6.36 (m, 1 H), 5.70-5.77 (m, 1 H), 5.58-5.88 (m, 1 H), 5.29 (br d, J = 6.7 Hz, 2 H), 4.90-4.95 (m, 2 H), 4.29- 4.67 (m, 2 H), 3.48-3.94 (m, 8 H), 3.39-3.47 (m, 3 H), 3.19- 3.28 (m, 1 H), 3.01-3.10 (m, 4 H), 2.71-2.88 (m, 3 H), 2.36- 2.53 (m, 8 H), 2.10-2.31 (m, 3 H), 1.88-2.00 (m, 1 H), 1.66 (br d, J = 12.5 Hz, 2 H); ¹⁹F NMR (376 MHz, CDC1₃) δ ppm −112.38-−108.46 (m, 2 F). | Intermediate E9 and B1; Step 4. Acrylic anhydride was used. Mixture of 6R and 6S diastereomers |
| 1-074 | 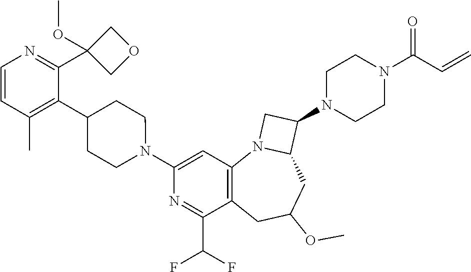 1-(4-((7aS,8R)-4-(difluoromethyl)- 6-methoxy-2-(4-(2-(3- methoxyoxetan-3-yl)-4- methylpyridin-3-yl)piperidin-1-yl)- 5,6,7,7a,8,9-hexahydroazeto[1,2- a]pyrido[3,4-f]azepin-8- yl)piperazin-1-yl)prop-2-en-1-one | m/z (ESI): 653.0 (M + H)⁺. ¹H NMR (400 MHz, CDCl₃) δ (ppm) 8.30 (d, J = 4.8 Hz, 1 H), 7.04 (d, J = 5.0 Hz, 1 H), 6.57 (dd, J = 16.8, 10.6 Hz, 1 H), 6.53 (t, J = 55.2 Hz, 1 H), 6.32 (dd, J = 16.9, 1.9 Hz, 1 H), 5.85 (s, 1 H), 5.70-5.77 (m, 1 H), 5.29 (br d, J = 6.7 Hz, 2 H), 4.93 (d, J = 7.3 Hz, 2 H), 4.43 (br dd, J = 42.7, 12.9 Hz, 2 H), 3.47-3.82 (m, 8 H), 3.46 (s, 3 H), 3.17-3.29 (m, 1 H), 3.07 (s, 3 H), 3.01-3.12 (m, 1 H), 2.72-2.88 (m, 3 H), 2.46 (s, 3 H), 2.34-2.52 (m, 5 H), 2.14-2.31 (m, 3 H), 1.87- 2.01 (m, 1 H), 1.66 (br d, J = 11.1 Hz, 2 H); ¹⁹F NMR (376 MHz, CDCl₃) δ (ppm) −115.10- −105.43 (m, 2 F). | Intermediate E9 and B1; The sample was purified via SFC using a Chiralcel OD, 2 × 25 cm 5 μm column with a mobile phase of 40% MeOH using a flowrate of 80 mL/min (2nd eluting isomer) |

### Method 4

### Example 75:1-(4-((7aS,8R)-4-(Difluoromethyl)-6,6-difluoro-2-(4-(2-(3-methoxy-3-oxetanyl)-4-methyl-3-pyridinyl)-1-piperidinyl)-5,6,7,7a,8,9-hexahydroazeto[1,2-a]pyrido[3,4-f]azepin-8-yl)-1-piperazinyl)-2-propen-1-one

### Example 76:1-(4-((7aS,8R)-4-(Difluoromethyl)-6-fluoro-2-(4-(2-(3-methoxy-3-oxetanyl)-4-methyl-3-pyridinyl)-1-piperidinyl)-7,7a,8,9-tetrahydroazeto[1,2-a]pyrido[3,4-f]azepin-8-yl)-1-piperazinyl)-2-propen-1-one

### Example 77:1-(4-((6R,7aS,8R)-4-(Difluoromethyl)-6-fluoro-2-(4-(2-(3-methoxy-3-oxetanyl)-4-methyl-3-pyridinyl)-1-piperidinyl)-5,6,7,7a,8,9-hexahydroazeto[1,2-a]pyrido[3,4-f]azepin-8-yl) ˜1-piperazinyl)-2-propen-1-one

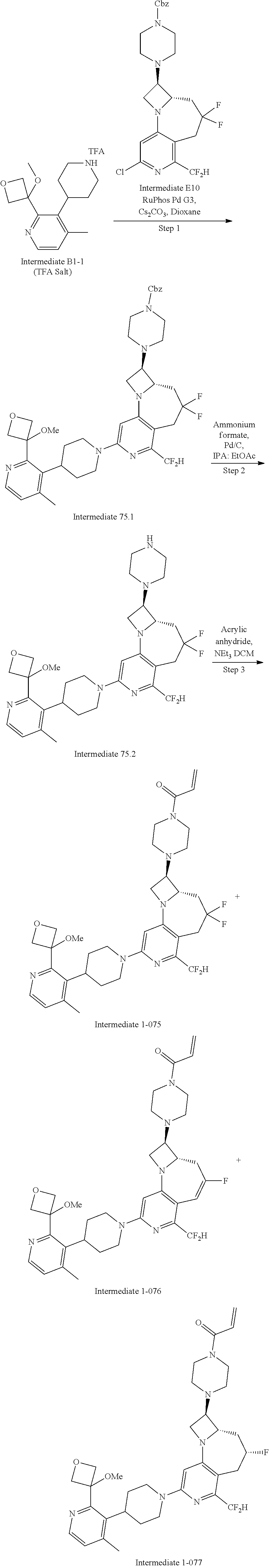

Step 1. Benzyl 4-((7aS,8R)-4-(difluoromethyl)-6,6-difluoro-2-(4-(2-(3-methoxyoxetan-3-yl)-4-methylpyridin-3-yl)piperidin-1-yl)-5,6,7,7a,8,9-hexahydroazeto[1,2-a]pyrido[3,4-f]azepin-8-yl)piperazine-1-carboxylate, Intermediate 75.1. To a solution of Intermediate B1-1 (TFA salt) (53 mg, 0.141 mmol) in 1,4-dioxane (1.3 mL), was added RuPhos Pd G3 (15 mg, 0.018 mmol, Strem Chemicals, Inc.), Cs₂CO₃ (110 mg, 0.34 mmol, Strem Chemicals, Inc.), and Intermediate E10 (58 mg, 0.113 mmol) and stirred at 100° C. for 1.5 h. The reaction mixture was quenched by water (1 mL) and extracted with EtOAc (3×1 mL). The combined organic extracts were washed with brine, dried over Na₂SO₄, and concentrated. The crude material was purified by column chromatography eluting with acetone in heptane (5-80%) to give Intermediate 75.1 (80 mg, 0.108 mmol), m/z (ESI): 738.8 (M+H)⁺.

Step 2. (7aS,8R)-4-(Difluoromethyl)-6,6-difluoro-2-(4-(2-(3-methoxyoxetan-3-yl)-4-methylpyridin-3-yl)piperidin-1-yl)-8-(piperazin-1-yl)-5,6,7,7a,8,9-hexahydroazeto[1,2-a]pyrido[3,4-f]azepine, Intermediate 75.2. To a solution of Intermediate 75.1 (78 mg, 0.108 mmol) in IPA (0.5 mL) and EtOAc (1.5 mL), was added ammonium formate (34.1 mg, 0.541 mmol), and 10 wt % palladium on activated carbon (17 mg, 0.016 mmol, Sigma-Aldrich Corporation). The reaction mixture was stirred at 45° C. for 1 h, filtered and concentrated to give Intermediate 75.2, m/z (ESI): 605.0 (M+H)⁺.

Step 3. 1-(4-((7aS,8R)-4-(Difluoromethyl)-6,6-difluoro-2-(4-(2-(3-methoxyoxetan-3-yl)-4-methylpyridin-3-yl)piperidin-1-yl)-5,6,7,7a,8,9-hexahydroazeto[1,2-a]pyrido[3,4-f]azepin-8-yl)piperazin-1-yl)prop-2-en-1-one, Compound 1-075. The reaction mixture from Step 2 was dissolved in DCM (2 mL), and Et; N (0.02 mL, 0.142 mmol), and 0.2 M acrylic anhydride solution in DCM (0.541 mL, 0.108 mmol, Sigma-Aldrich Corporation) at 0° C. was added. The reaction mixture was stirred for 15 min. The crude material was purified by column chromatography, eluting with acetone in heptane (15%-70%). The crude material was purified by reverse-phase preparative HPLC using a Phenomenex Gemini column, 10 micron, C18, 100 Å, 150×30 mm, 0.1% TEA in ACN/water, gradient 10% to 90% over 15 min to obtain a Is eluting isomer, a 2ªd eluting isomer, and a 3ª eluting isomer.

The identity of the isomers was assigned to be Compound 1-075 (20 mg, 0.030 mmol, 28% yield) as the 1^{st} eluting isomer, Compound 1-076 (20 mg, 0.031 mmol 29% yield) as the 2ªd eluting isomer, and Compound 1-077 (10 mg, 0.016 mmol, 3% yield) as the 314 eluting isomer. 1^{st} Eluting isomer: m/z (ESI): 659.0 (M+H)⁺. ¹H NMR (400 MHz, CDCl₃) δ (ppm) 8.31 (d, J=5.0 Hz, 1H), 7.04 (d, J=5.0 Hz, 1H), 6.57 (dd, J=16.8, 10.6 Hz, 1H), 6.50 (t, J=55.0 Hz, 1H), 6.32 (dd, J=16.8, 1.8 Hz, 1H), 5.79 (s, 1H), 5.73 (dd, J=10.7, 1.9 Hz, 1H), 5.29 (d, J=6.9 Hz, 2H), 4.93 (d, J=6.9 Hz, 2H), 4.39-4.51 (m, 2H), 4.00 (br dd, J=11.4, 5.3 Hz, 1H), 3.74-3.88 (m, 3H), 3.49 ˜ 3.71 (m, 4H), 3.10-3.24 (m, 1H), 3.08 (s, 3H), 3.04 (q, J=6.5 Hz, 1H), 2.73-2.89 (m, 3H), 2.46 (s, 4H), 2.13-2.27 (m, 2H), 2.07-2.55 (m, 3H), 1.67 (br d, J=12.1 Hz, 2H), 19F NMR (376 MHz, CDCl₃) δ (ppm) −99.06-−87.55 (m, 2F), −111.67-−108.08 (m, 2F); 2^{nd} Eluting isomer: m/z (ESI): 639.0 (M+H)⁺. ¹H NMR (400 MHz, CDCl₃) δ (ppm) 8.33 (d, J=5.0 Hz, 1H), 8.30 (d, J=4.8 Hz, 1H), 7.04 (d, J=5.0 Hz, 1H), 6.75 (br d, J=27.4 Hz, 1H), 6.53-6.61 (m, 1H), 6.52 (br t, J=54.9 Hz, 1H), 6.32 (dd, J=16.8, 1.7 Hz, 1H), 5.74 (dd, J=10.6, 1.7 Hz, 1H), 5.69 (s, 1H), 5.29 (br d, J=6.8 Hz, 2H), 4.93 (d, J=6.8 Hz, 2H), 4.45 (br dd, J=41.7, 12.9 Hz, 2H), 3.98 (t, J=6.8 Hz, 1H), 3.89-3.96 (m, 1H), 3.50-3.85 (m, 6H), 3.07 (s, 3H), 3.02-3.10 (m, 2H), 2.73-2.90 (m, 4H), 2.45 (s, 3H), 2.35-2.55 (m, 2H), 2.21-2.29 (m, 1H), 2.12-2.19 (m, 1H), 1.67 (br s, 2H); ¹⁹F NMR (376 MHz, CDCl₃) δ (ppm) −90.03-−89.46 (m, 1 F), −113.14-−109.71 (m, 2 F); 3^{rd} Eluting isomer: m/z (ESI): =641.4 (M+H)⁺. ¹H NMR (500 MHz, DMSO-d₆) δ (ppm) 8.30 (d, J=4.8 Hz, 1H), 7.19 (d, J=5.1 Hz, 1H), 6.85 (dd, J=16.7, 10.4 Hz, 1H), 6.65-6.91 (m, 1H), 6.18 (dd. J=16.7, 2.1 Hz, 1H), 6.02 (s, 1H), 5.71-5.83 (m, 1H), 5.15 (br d, J=6.9 Hz, 2H), 4.81 (d. J=7.3 Hz, 3H), 4.45 (br t, J=13.1 Hz, 2H), 3.57-4.17 (m, 6H), 3.16-3.31 (m, 1H), 2.96 (s, 3H), 2.92-3.09 (m, 3H), 2.63-2.90 (m, 5H), 2.53-2.57 (m, 1H), 2.41-2.47 (m, 1H), 2.39 (s, 3H), 2.01-2.17 (m, 3H), 1.61 (br d, J=11.9 Hz, 2H).

Compounds in Table 2-4 were prepared following the procedure described in Method 4, using appropriate starting materials. All starting materials are commercially available or are described in the Intermediates section above.

TABLE 2-4

| Compound | | LCMS: (ESI + ve ion) m/z; | |
|---|---|---|---|
| # | Chemical Structure & Name | NMR | Comments |
| 1-078 | 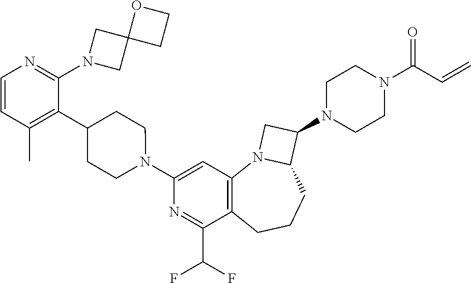 [SMILES: C=CC(=O)N1CCN([C@@H]2CN3c4cc(N5CCC(c6c(C)ccnc6N6CC7(CCO7)C6)CC5)nc(C(F)F)c4CCC[C@@H]23)CC1] 1-(4-((7aS,8R)-4-(difluoromethyl)-2- (4-(4-methyl-2-(1-oxa-6- azaspiro[3.3]heptan-6-yl)-3-pyridinyl)- 1-piperidinyl)-5,6,7,7a,8,9- hexahydroazeto[1,2-a]pyrido[3,4- f]azepin-8-yl)-1-piperazinyl)-2- propen-1-one | m/z (ESI): 634.0 (M + H)⁺. ¹H NMR (400 MHz, DMSO- d₆) δ (ppm) 7.87 (d, J = 4.8 Hz, 1 H), 6.51-6.86 (m, 3 H), 6.10 (dd, J = 16.7, 2.3 Hz, 1 H), 5.94 (s, 1 H), 5.64-5.71 (m, 1 H), 4.42 (br t, J = 7.4 Hz, 4 H), 4.18 (br d, J = 9.8 Hz, 2 H), 4.06 (d, J = 9.4 Hz, 2 H), 3.66- 3.78 (m, 3 H), 3.56 (br s, 4 H), 2.86 (br t, J = 7.4 Hz, 7 H), 2.31- 2.39 (m, 5 H), 2.28 (s, 3 H), 2.02-2.15 (m, 2 H), 1.89 (br s, 3 H), 1.60 (br d, J = 12.3 Hz, 2 H), 1.45 (br s, 1 H). | Intermediates E1 and B11 were used; Step 2. IPA was used as solvent |
| 1-079 | 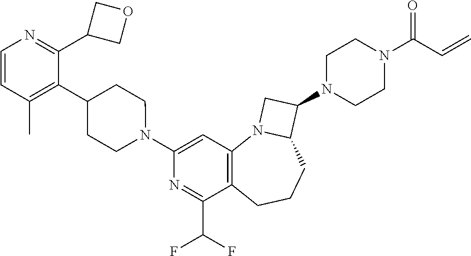 [SMILES: C=CC(=O)N1CCN([C@@H]2CN3c4cc(N5CCC(c6c(C)ccnc6C6COC6)CC5)nc(C(F)F)c4CCC[C@@H]23)CC1] 1-(4-((7aS,8R)-4-(difluoromethyl)-2- (4-(4-methyl-2-(3-oxetanyl)-3- pyridinyl)-1-piperidinyl)-5,6,7,7a,8,9- hexahydroazeto[1,2-a]pyrido[3,4- f]azepin-8-yl)-1-piperazinyl)-2- propen-1-one | m/z (ESI): 593.0 (M + H)⁺. ¹H NMR (400 MHz, DMSO- d₆) δ (ppm) 8.31 (d, J = 4.8 Hz, 1 H), 7.07 (d, J = 4.8 Hz, 1 H), 6.57-6.88 (m, 2 H), 6.04- 6.17 (m, 1 H), 5.95 (s, 1 H), 5.62-5.71 (m, 1 H), 4.89 (br d, J = 6.7 Hz, 2 H), 4.82 (br d, J = 4.6 Hz, 2 H), 4.61-4.73 (m, 1 H), 4.36-4.47 (m, 2 H), 3.66-3.77 (m, 3 H), 3.55 (br d, J = 1.3 Hz, 4 H), 3.03 (br dd, J = 12.6, 9.3 Hz, 1 H), 2.77- 2.96 (m, 4 H), 2.32-2.39 (m, 8 H), 1.75-2.04 (m, 5 H), 1.58 (br d, J = 12.1 Hz, 2 H), 1.45 (br s, 1 H). | Intermediates E1 and B16 were used; Step 2. IPA was used as solvent |
| 1-080 | 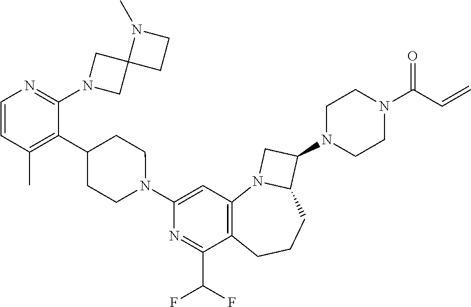 [SMILES: C=CC(=O)N1CCN([C@@H]2CN3c4cc(N5CCC(c6c(C)ccnc6N6CC7(CCN7C)C6)CC5)nc(C(F)F)c4CCC[C@@H]23)CC1] 1-(4-((7aS,8R)-4-(difluoromethyl)-2- (4-(4-methyl-2-(1-methyl-1,6- diazaspiro[3.3]heptan-6-yl)-3- pyridinyl)-1-piperidinyl)-5,6,7,7a.8,9- hexahydroazeto[1,2-a]pyrido[3,4- f]azepin-8-yl)-1-piperazinyl)-2- propen-1-one | m/z (ESI): 647.1 (M + H)⁺. ¹H NMR (400 MHz, DMSO- d₆) δ (ppm) 7.87 (d, J = 5.0 Hz, 1 H), 6.68-6.86 (m, 2 H), 6.60 (d, J = 5.0 Hz, 1 H), 6.10 (dd, J = 16.6, 2.4 Hz, 1 H), 5.95 (s, 1 H), 5.62-5.72 (m, 1 H), 4.38-4.48 (m, 2 H), 4.09 (dd, J = 8.5, 0.9 Hz, 2 H), 3.85 (br d, J = 8.8 Hz, 2 H), 3,69-3.78 (m, 3 H), 3.56 (br s, 5 H), 3.03 (s, 4 H), 2.78-2.94 (m, 3 H), 2.34 (br dd, J = 3.8, 1.7 Hz, 6 H), 2.28 (s, 3 H), 2.20 (s, 3 H), 2.05-2.09 (m, 2 H), 1.70- 1.93 (m, 4 H), 1.60 (br d, J = 10.7 Hz, 2 H). | Intermediates E1 and B13 were used; Step 2. IPA was used as solvent |
| 1-081 | 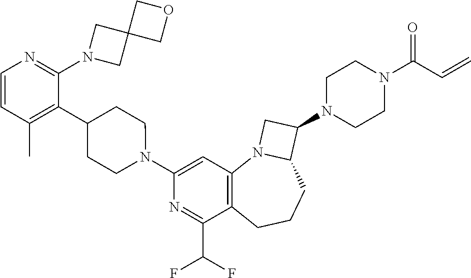 [SMILES: C=CC(=O)N1CCN([C@@H]2CN3c4cc(N5CCC(c6c(C)ccnc6N6CC7(COC7)C6)CC5)nc(C(F)F)c4CCC[C@@H]23)CC1] 1-(4-((7aS,8R)-4-(difluoromethyl)-2- (4-(4-methyl-2-(2-oxa-6- azaspiro[3.3]heptan-6-yl)-3-pyridinyl)- 1-piperidinyl)-5,6,7,7a,8,9- hexahydroazeto[1,2-a]pyrido[3,4- f]azepin-8-yl)-1-piperazinyl)-2- propen-1-one | m/z (ESI): 634.150 (M + H)+. ¹H NMR (500 MHz, CDCl₃) δ ppm 7.96 (d, J = 5.1 Hz, 1 H), 6.40-6.65 (m, 3 H), 6.32 (dd, J = 16.8, 1.9 Hz, 1 H), 5.82 (s, 1 H), 5.73 (dd, J = 10.5, 1.8 Hz, 1 H), 4.85 (s, 4 H), 4.41-4.50 (m, 2 H), 4.21 (s, 4 H), 3.66- 3.81 (m, 5 H), 3.61 (br s, 2 H), 3.23 (br dd, J = 14.9, 8.5 Hz, 1 H), 2.95-3.04 (m, 2 H), 2.82- 2.89 (m, 2 H), 2.40-2.50 (m, 5 H), 2.37 (s, 3 H), 2.20-2.30 (m, 2 H), 1.92-2.03 (m, 2 H), 1.87 (br dd, J = 12.3, 3.7 Hz, 1 H), 1.68-1.74 (m, 2 H), 1.42- 1.50 (m, 1H). ¹⁹F NMR (471 MHz, CDCl₃) δ ppm −111.57-109.67 (m, 2 F) | Intermediates E1 and B9 were used: Step 2. IPA and THF were used as solvent |
| 1-082 | 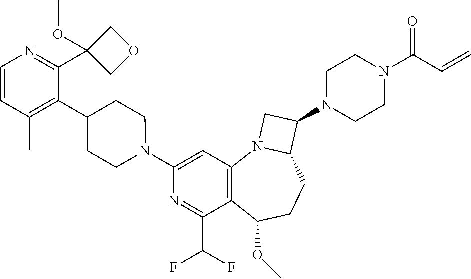 [SMILES: C=CC(=O)N1CCN([C@@H]2CN3c4cc(N5CCC(c6c(C)ccnc6C6(OC)COC6)CC5)nc(C(F)F)c4[C@@H](OC)CC[C@@H]23)CC1] 1-(4-((5S,7aS,8R)-4-(difluoromethyl)- 5-methoxy-2-(4-(2-(3-methoxy-3- oxetanyl)-4-methyl-3-pyridinyl)-1- piperidinyl)-5,6,7,7a,8,9- hexahydroazeto[1,2-a]pyrido[3,4- f]azepin-8-yl)-1-piperazinyl)-2- propen-1-one | m/z (ESI): 653.0 (M + H)⁺. ¹H NMR (400 MHz, CDCl₃) δ ppm 8.30 (d, J = 4.8 Hz, 1 H), 7.04 (d, J = 5.0 Hz, 1 H), 6.38- 6.68 (m, 2 H), 6.30 (dd, J = 16.7, 1.9 Hz, 1 H), 5.79 (s, 1 H), 5.72 (dd, J = 10.6, 2.0 Hz, 1 H), 5.29 (dd, J = 6.9, 1.9 Hz, 2 H), 5.01 (br d, J = 5.9 Hz, 1 H), 4.93 (d, J = 7.1 Hz, 2 H), 4.55 (br d, J = 13.2 Hz, 1 H), 4.32- 4.41 (m, 1 H), 3.82 (t, J = 6.6 Hz, 1 H), 3.55-3.77 (m. 4 H), 3.47-3.55 (m, 2 H), 3.19 (s, 3 H), 3.08 (s, 3 H), 3.05-3.11 (m, 1 H), 2.75-2.91 (m, 3 H), 2.45 (s, 3 H), 2.42 (br s, 4 H), 2.20-2.29 (m, 3 H), 2.19 (s, 3 H), 1.63-1.70 (m, 2 H). ¹⁹F NMR (376 MHz, CDCl₃) δ (ppm) −109.17-−106.27 (m, 2 F) | Intermediates E6 and B1 were used |
| 1-084 | 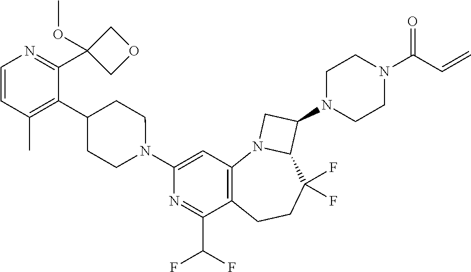 [SMILES: C=CC(=O)N1CCN([C@@H]2CN3c4cc(N5CCC(c6c(C)ccnc6C6(OC)COC6)CC5)nc(C(F)F)c4CCC(F)(F)[C@@H]23)CC1] 1-(4-((7aR,8R)-4-(difluoromethyl)-7,7- difluoro-2-(4-(2-(3-methoxy-3- oxetanyl)-4-methyl-3-pyridinyl)-1- piperidinyl)-5,6,7,7a,8,9- hexahydroazeto[1,2-a]pyrido[3,4- f]azepin-8-yl)-1-piperazinyl)-2- propen-1-one | m/z (ESI): 659.0 (M + H)⁺ ¹H NMR (400 MHz, CDCl₃) δ (ppm) 8.31 (d, J = 4.8 Hz, 1 H), 7.04 (d, J = 4.8 Hz, 1 H), 6.57 (dd, J = 16.8, 10.6 Hz, 1 H), 6.34-6.62 (m, 1 H), 6.31 (dd, J = 16.9, 1.9 Hz, 1 H), 5.83 (s, 1 H), 5.73 (dd, J = 10.5. 1.9 Hz, 1 H), 5.29 (d, J = 6.7 Hz, 2 H), 4.93 (d, J = 7.3 Hz, 2 H), 4.34- 4.51 (m, 2 H), 4.05 (br dd, J = 23.5, 5.7 Hz, 1 H), 3.85 (br t, J = 6.8 Hz, 2 H), 3.47-3.75 (m, 6 H), 3.05-3.15 (m, 4 H), 2.74-2.88 (m, 4 H), 2.35- 2.55 (m, 7 H), 2.20-2.27 (m, 2 H), 2.00-2.12 (m, 1 H), 1.67 (br d, J = 12.3 Hz, 2 H). ¹⁹F NMR (376 MHz. CDCl₃) δ (ppm) −99.21 (dt, J = 240.4, 12.0 Hz, 1 F), −110.57- −109.48 (m, 2 F), −110.86 (br dd, J = 55.1. 13.4 Hz, 1 F); | Intermediates E7 and B1 |
| 1-085 | 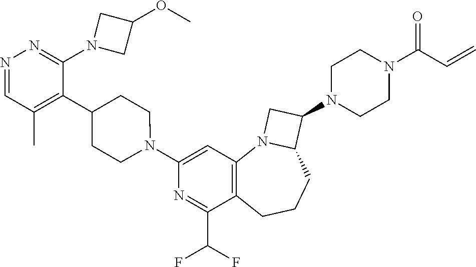 [SMILES: C=CC(=O)N1CCN([C@@H]2CN3c4cc(N5CCC(c6c(C)cnnc6N6CC(OC)C6)CC5)nc(C(F)F)c4CCC[C@@H]23)CC1] 1-(4-((7aS,8R)-4-(difluoromethyl)-2- (4-(3-(3-methoxy-1-azetidinyl)-5- methyl-4-pyridazinyl)-1-piperidinyl)- 5,6,7,7a,8,9-hexahydroazeto[1,2- a]pyrido[3,4-f]azepin-8-yl)-1- piperazinyl)-2-propen-1-one | m/z (ESI): 623.0 (M + H)⁺. ¹H NMR (500 MHz, CDCl₃) δ (ppm) 8.42 (s, 1 H), 6.57 (dd, J = 16.8, 10.6 Hz, 1 H), 6.51 (t, J = 55.2 Hz, 1 H), 6.32 (dd, J = 16.9, 1.8 Hz, 1 H), 5.79 (s, 1 H), 5.71-5.76 (m, 1 H), 4.30- 4.51 (m, 5 H), 4.12 (dd, J = 9.0, 3.7 Hz, 2 H), 3.66-3.82 (m, 5 H), 3.61 (br s, 2 H), 3.38 (s, 3 H), 3.18-3.25 (m, 1 H), 2.92- 3.01 (m, 2 H), 2.82 (tdd, J = 12.4, 12.4, 6.2, 2.4 Hz, 2 H). 2.37-2.51 (m, 5 H), 2.35 (s, 3 H), 2.13-2.25 (m, 2 H), 1.92- 2.03 (m, 2 H), 1.82-1.91 (m, 1 H), 1.72 (br d, J = 13.0 Hz, 2 H), 1.41-1.51 (m, 1 H); ¹⁹F NMR (471 MHz, CDCl₃) δ (ppm) −115.70-−107.02 (m, 2 F). | Intermediates E1 and B46 were used |
| 1-107 | 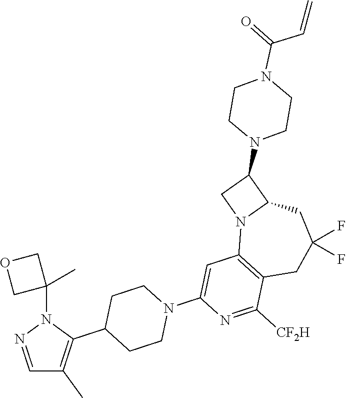 [SMILES: C=CC(=O)N1CCN([C@@H]2CN3c4cc(N5CCC(c6c(C)cnn6C6(C)COC6)CC5)nc(C(F)F)c4CC(F)(F)C[C@@H]23)CC1] 1-(4-((7aS,8R)-4-(difluoromethyl)-6,6- difluoro-2-(4-(4-methyl-1-(3- methyloxetan-3-yl)-1H-pyrazol-5- yl)piperidin-1-yl)-5,6,7,7a,8,9- hexabydroazeto[1,2-a]pyrido[3,4- f]azepin-8-yl)piperazin-1-yl)prop-2- en-1-one | m/z (ESI): 632.2 (M + H)⁺. ¹H NMR (400 MHz, CDCl₃) δ (ppm) 7.25 (s, 1 H), 6.57 (dd, J = 16.8, 10.6 Hz, 1 H), 6.36- 6.66 (m, 1 H), 6.32 (dd, J = 16.8, 1.8 Hz, 1 H), 5.79 (s, 1 H), 5.74 (dd, J = 10.6, 1.8 Hz, 1 H), 5.27 (d, J = 6.3 Hz, 2 H), 4.67 (d, J = 6.5 Hz, 2 H), 4.47 (br t, J = 14.2 Hz, 2 H), 4.00 (br dd, J = 11.4, 5.5 Hz, 1 H), 3.76- 3.88 (m, 3 H), 3.49-3.72 (m, 4 H), 3.08-3.25 (m, 1 H), 3.04 (q, J = 6.5 Hz, 1 H), 2.69- 2.81 (m, 2 H), 2.31-2.55 (m, 6 H), 2.19-2.30 (m, 1 H), 2.02-2.14 (m, 5 H), 1.84 (s, 3 H), 1.81 (br d, J = 13.2 Hz, 2 H); ¹⁹F NMR (471 MHz, CDCl₃) δ (ppm) −89.09 (br dd, J = 243.2, 23.0 Hz, 1 F), −96.08 (br d, J = 239.3 Hz, 1 F), −115.25-−106.89 (m, 2 F). | Intermediate E10 and B6 were used |
| 1-108 | 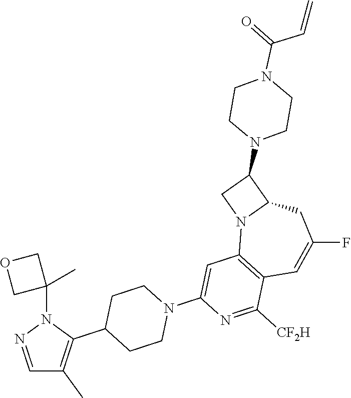 [SMILES: C=CC(=O)N1CCN([C@@H]2CN3c4cc(N5CCC(c6c(C)cnn6C6(C)COC6)CC5)nc(C(F)F)c4C=C(F)C[C@@H]23)CC1] 1-(4-((7aS,8R)-4-(difluoromethyl)-6- fluoro-2-(4-(4-methyl-1-(3- methyloxetan-3-yl)-1H-pyrazol-5- yl)piperidin-1-yl)-7,7a,8,9- tetrahydroazeto[1,2-a]pyrido[3,4- f]azepin-8-yl)piperazin-1-yl)prop-2- en-1-one | m/z (ESI): 612.2 (M + H)⁺. ¹H NMR (400 MHz, CDCl₃) δ (ppm) 7.24 (s, 1 H), 6.75 (br d, J = 27.4 Hz, 1 H), 6.53-6.61 (m, 1 H), 6.53 (t, J = 55.0 Hz, 1 H), 6.31 (dd, J = 16.7, 1.9 Hz, 1 H), 5.73 (dd, J = 10.7, 1.9 Hz, 1 H), 5.68 (s, 1 H), 5.27 (d, J = 6.1 Hz, 2 H), 4.67 (d, J = 6.5 Hz, 2 H), 4.39-4.54 (m, 2 H), 3.89-4.02 (m, 2 H), 3.44- 3.88 (m, 5 H), 2.99-3.13 (m, 2 H), 2.70-2.89 (m, 3 H), 2.44 (br d, J = 2.9 Hz, 4 H), 2.20-2.30 (m, 1 H), 2.07 (s, 3 H), 1.98-2.14 (m, 2 H), 1.84 (s, 3 H), 1.80 (br d, J = 13.2 Hz, 2 H); ¹⁹F NMR (471 MHz, CDCl₃) δ (ppm) −89.33 (ddd, J = 27.1, 11.1, 3.5 Hz, 1 F), −113.09- −109.34 (m, 2 F). | Intermediate E10 and B6 were used |

### Method 5

### Example 86:1-(4-((7aS,8R)-4-(Difluoromethyl)-2-(4-(5-methyl-3-(2-oxa-6-azaspiro[3.3]heptan-6-yl)-4-pyridazinyl)-1-piperidinyl)-7,7a,8,9-tetrahydroazeto[1,2-a]pyrido[3,4-f]azepin-8-yl)-1-piperazinyl)-2-propen-1-one

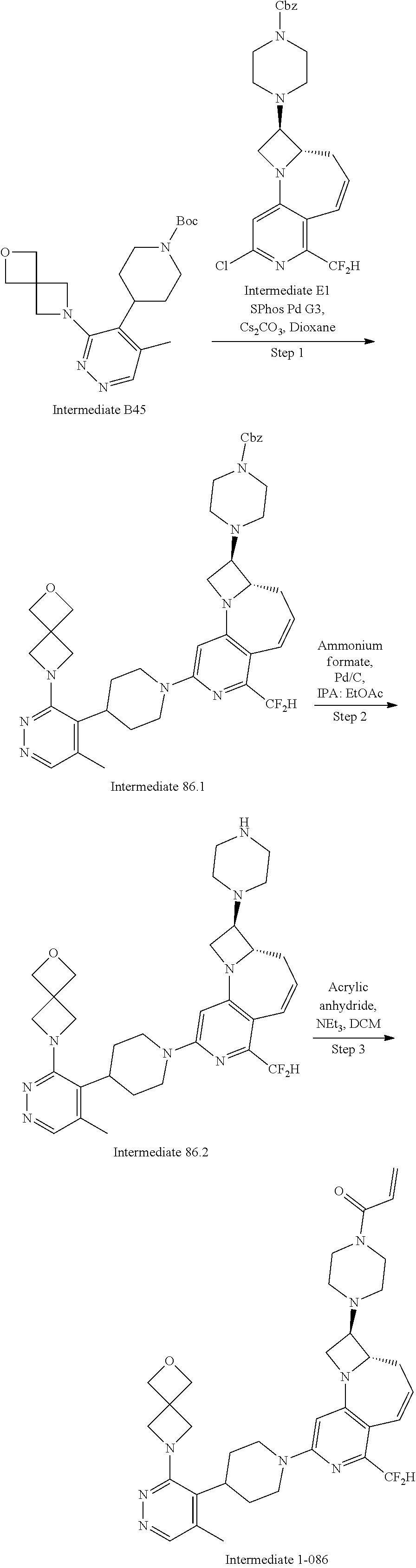 [SMILES: Cc1cnnc(N2CC3(COC3)C2)c1C1CCN(C(=O)OC(C)(C)C)CC1 ; O=C(OCc1ccccc1)N1CCN([C@@H]2CN3c4cc(Cl)nc(C(F)F)c4C=CC[C@@H]23)CC1 ; Cc1cnnc(N2CC3(COC3)C2)c1C1CCN(c2cc3c(c(C(F)F)n2)C=CC[C@H]2[C@H](N4CCN(C(=O)OCc5ccccc5)CC4)CN32)CC1 ; Cc1cnnc(N2CC3(COC3)C2)c1C1CCN(c2cc3c(c(C(F)F)n2)C=CC[C@H]2[C@H](N4CCNCC4)CN32)CC1 ; C=CC(=O)N1CCN([C@@H]2CN3c4cc(N5CCC(c6c(C)cnnc6N6CC7(COC7)C6)CC5)nc(C(F)F)c4C=CC[C@@H]23)CC1]

Step 1. Benzyl 4-((7aS,8R)-4-(difluoromethyl)-2-(4-(5-methyl-3-(2-oxa-6-azaspiro[3.3]heptan-6-yl)pyridazin-4-yl)piperidin-1-yl)-7,7a,8,9-tetrahydroazeto[1,2-a]pyrido[3,4-f]azepin-8-yl)piperazine-1-carboxylate, Intermediate 86.1. Intermediate B45 (295 mg, 1.07 mmol) was dissolved in degassed dioxane (8 mL), and S Phos Pd G3 (77 mg, 0,089 mmol, Sigma-Aldrich Corporation), Cs₂CO₃ (1458 mg, 4.47 mmol), and Intermediate E1 (425 mg, 0.895 mmol) were added. The reaction mixture was degassed by sparging with N₂ for 15 min and, then, heated to 80° C. for 3 h. The reaction mixture was diluted with water and EtOAc, and the layers were separated. The aqueous layer was extracted with EtOAc, and the combined organic extracts were washed with brine, dried over Na₂SO₄, filtered, and concentrated. The crude material was purified by column chromatography, eluting with a gradient of 0% to 70% (3:1 EtOAc/EtOH) in heptane to afford Intermediate 86.1 (300 mg. 0.421 mmol, 47% yield), m/z (ESI): 713.4 (M+H)⁺.

Step 2. 6-(4-(1-((7aS,8R)-4-(Difluoromethyl)-8-(piperazin-1-yl)-7,7a,8,9-tetrahydroazeto[1,2-a]pyrido[3,4-f]azepin-2-yl)piperidin-4-yl)-5-methylpyridazin-3-yl)-2-oxa-6-azaspiro[3.3]heptane, Intermediate 86.2. A solution of Intermediate 86.1 (120 mg, 0.168 mmol) in ^{t}PrOH (15 mL) was purged with N₂ followed by the addition of ammonium formate (106 mg, 1.683 mmol, Fisher Scientific), and palladium on activated carbon (10 wt %, 36 mg, 0.034 mmol, Oakwood Products, Inc.) and the resulting mixture was heated at 50° C. for 20 min. The reaction mixture was filtered, concentrated, diluted with brine, and extracted with DCM. The combined organics were dried over Na₂SO₄, filtered, concentrated, and purified by chromatography, eluting with a gradient of 0-25% MeOH in DCM to afford Intermediate 86.2, m/z (ESI): 579.4 (M+H) 1. ¹H NMR (500 MHz, CDCl₃) δ (ppm) 8.44 (s, 1H), 6.80-6.91 (m, 1H), 6.41-6.72 (m, 1H), 5.75-5.81 (m, 1H), 5.63 (S. 1H), 4.88 (s, 4H), 4.44-4.57 (m, 2H), 4.37 (s, 4H), 3.94-4.00 (m, 2H), 3.70 (t, J=7.1 Hz, 1H), 2.82-3.02 (m, 9H), 2.72-2.79 (m, 1H), 2.62-2.70 (m, 1H), 2.32-2.43 (m, 7H), 2.13-2.28 (m, 2H), 1.68-1.75 (m, 2H). IF NMR (471 MHz, CDCl₃) δ (ppm) −111.89-−110.04 (m, 2 F)

Step 3. 1-(4-((7aS,8R)-4-(Difluoromethyl)-2-(4-(5-methyl-3-(2-oxa-6-azaspiro[3.3]heptan-6-yl)pyridazin-4-yl)piperidin-1-yl)-7,7a,8,9-tetrahydroazeto[1,2-a]pyrido[3,4-f]azepin-8-yl)piperazin-1-yl)prop-2-en-1-one, Compound 1-086. To a solution of Intermediate 86.2 in DCM (10 mL) at rt was added dropwise acryloyl chloride 0.2 M in DCM (0.842 mL, 0.168 mmol, Sigma Aldrich), and the resulting mixture was stirred for 10 min, concentrated and purified by chromatography, eluting with a gradient of 0-20% MeOH in DCM to afford 1-(4-((7aS,8R)-4-(difluoromethyl)-2-(4-(5-methyl-3-(2-oxa-6-azaspiro[3.3|heptan-6-yl)-4-pyridazinyl)-1-piperidinyl)-7,7a,8,9-tetrahydroazeto[1,2-a]pyrido[3,4-f]azepin-8-yl)-1-piperazinyl)-2-propen-1-one, Compound 1-086 (42 mg, 0.066 mmol, 39% yield), m/z (ESI): 633.3 (M+H)⁺. ¹H NMR (500 MHz, CDCl₃) δ (ppm) 8.44 (s, 1H), 6.86 (br d, J=12.5 Hz, 1H), 6.43-6.71 (m, 2H), 6.32 (dd, J=16.7, 1.8 Hz, 1H), 5.72-5.81 (m, 2H), 5.63 (s, 1H), 4.88 (s, 4H), 4.52-4.61 (m, 1H), 4.42-4.51 (m, 1H), 4.37 (s, 4H), 3.96-4.02 (m, 2H), 3.54-3.86 (m, 5H), 2.84-3.04 (m, 4H), 2.71-2.80 (m, 1H), 2.61-2.70 (m, 1H), 2.38-2.51 (m, 4H), 2.34 (s, 3H), 2.13-2.26 (m, 2H), 1.67-1.77 (m, 2H); ¹⁹F NMR (471 MHz, CDCl₃) δ (ppm) −111.85-−110.09 (m, 2 F).

Compounds in Table 2-5 were prepared following the procedure described in Method 5, using appropriate starting materials. All starting materials are commercially available or are described in the Intermediates section above.

TABLE 2-5

| Compound | | LCMS: (ESI + ve ion) m/z; | |
|---|---|---|---|
| # | Chemical Structure & Name | NMR | Comments |
| 1-087 | 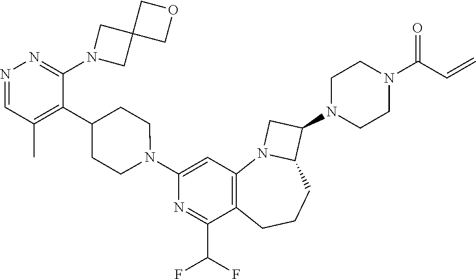 [SMILES: C=CC(=O)N1CCN([C@@H]2CN3c4cc(N5CCC(c6c(C)cnnc6N6CC7(COC7)C6)CC5)nc(C(F)F)c4CCC[C@@H]23)CC1] 1-(4-((7aS,8R)-4-(difluoromethyl)-2- (4-(5-methyl-3-(2-oxa-6- azaspiro[3.3]heptan-6-yl)-4- pyridazinyl)-1-piperidinyl)- 5,6,7,7a,8,9-hexahydroazeto[1,2- a]pyrido[3,4-f]azepin-8-yl)-1- piperazinyl)-2-propen-1-one | m/z (ESI): 635.4 (M + H)⁺; ¹H NMR (500 MHz, CDCl₃) δ (ppm) 8.44 (s, 1 H), 6.40- 6.64 (m, 2 H), 6.29-6.36 (m, 1 H), 5.79-5.82 (m, 1 H), 5.71-5.76 (m, 1 H), 4.87 (s, 4 H), 4.43-4.51 (m, 2 H), 4.36 (s, 4 H), 3.66-3.81 (m, 5 H), 3.61 (br s, 2H), 3.22 (br dd, J = 14.2, 8.8 Hz, 1 H), 2.91- 2.99 (m, 2 H), 2.85 (tdd, J = 12.4, 12.4, 5.4, 2.2 Hz, 2 H), 2.39-2.50 (m, 5 H), 2.35 (s, 3 H), 2.15-2.26 (m, 2 H), 1.94- 2.03 (m, 2 H), 1.87 (br dd, J = 12.5, 3.9 Hz, 1 H), 1.71 (br d, J = 12.1 Hz, 2 H), 1.47 (br dd, J = 11.7, 2.2 Hz, 1 H); ¹⁹F NMR (471 MHz, CDCl₃) δ (ppm) −111.54-−109.69 (m, 2 F) | Intermediate E8 was used |
| 1-088 | 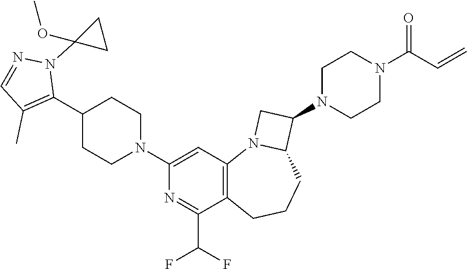 [SMILES: C=CC(=O)N1CCN([C@@H]2CN3c4cc(N5CCC(c6c(C)cnn6C6(OC)CC6)CC5)nc(C(F)F)c4CCC[C@@H]23)CC1] 1-(4-((7aS,8R)-4-(difluoromethyl)-2- (4-(1-(1-methoxycyclopropyl)-4- methyl-1H-pyrazol-5-yl)-1- piperidinyl)-5,6,7,7a,8,9- hexahydroazeto[1,2-a]pyrido[3,4- f]azepin-8-yl)-1-piperazinyl)-2- propen-1-one | m/z (ESI): 596.0 (M + H)⁺. ¹H NMR (CDCl₃, 400 MHz) δ (ppm) 7.20 (s, 1 H), 6.34- 6.69 (m, 2 H), 6.29 (dd, J = 16.7, 1.9 Hz, 1 H), 5.79 (s, 1 H), 5.71 (dd, J = 10.5, 1.9 Hz, 1 H), 4.41 (br t, J = 15.9 Hz, 2 H), 3.57-3.83 (m, 7 H), 3.33- 3.49 (m, 1 H), 3.21-3.25 (m, 1 H), 3.19-3.20 (m, 3 H), 2.95 (q, J = 6.6 Hz, 1 H), 2.74- 2.91 (m, 2 H), 2.36-2.50 (m, 5 H), 2.07 (s, 3 H), 1.87-2.06 (m, 5 H), 1.79-1.87 (m, 3 H), 1.36 (s, 4 H); ¹⁹F NMR (CDCl₃, 376 MHz) δ (ppm) −122.3-−103.9 (m, 2F) | Intermediates E1 and B4 were used; |
| 1-089 | 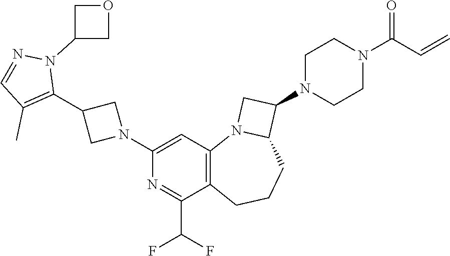 [SMILES: C=CC(=O)N1CCN([C@@H]2CN3c4cc(N5CC(c6c(C)cnn6C6COC6)C5)nc(C(F)F)c4CCC[C@@H]23)CC1] 1-(4-((7aS,8R)-4-(difluoromethyl)-2- (3-(4-methyl-1-(3-oxetanyl)-1H- pyrazol-5-yl)-1-azetidinyl)- 5,6,7,7a,8,9-hexahydroazeto[1,2- a]pyrido[3,4-f]azepin-8-yl)-1- piperazinyl)-2-propen-1-one | m/z (ESI): 554.0 (M + H)⁺. ¹H NMR (CDCl₃, 400 MHz) (ppm) 7.41 (s. 1 H), 6.35- 6.66 (m, 2 H), 6.30 (dd, J = 16.9, 1.8 Hz, 1 H), 5.72 (dd, J = 10.5, 1.8 Hz, 1 H), 5.54 (quin, J = 7.0 Hz, 1 H), 5.42 (s, 1 H), 5.21 (t, J = 6.3 Hz, 2 H), 4.97 (t, J = 6.9 Hz, 2 H), 4.38 (t, J = 8.2 Hz, 2 H), 4.06 (t, J = 7.0 Hz, 2 H), 3.92-4.01 (m, 1 H), 3.59-3.81 (m, 7 H), 3.14- 3.22 (m, 1 H), 2.95 (q, J = 6.6 Hz, 1 H), 2.38-2.54 (m, 5 H), 2.11 (s, 3 H), 1.81-2.03 (m, 3 H), 1.43-1.54 (m, 1 H); ¹⁹F NMR (CDCl₃, 376 MHz) δ (ppm) −110.68 (br d, 2F, J = 46.8 Hz) | Intermediates E1 and B34 were used; |
| 1-090 | 1-(4-((7aS,8R)-4-(difluoromethyl)-2- (4-(4-methyl-2-(2-oxa-6- azaspiro[3.3]heptan-6-yl)-3-pyridinyl)- 1-piperidinyl)-7,7a,8,9- tetrahydroazeto[1,2-a]pyrido[3,4- f]azepin-8-yl)-1-piperazinyl)-2- propen-1-one | m/z (ESI): 632.4 (M + H)⁺. ¹H NMR (400 MHz, MeOH- d4) δ (ppm) 7.84 (d, J = 5.2 Hz, 1 H), 6.41-6.86 (m, 4 H), 6.16-6.29 (m. 1 H), 5.70- 5.82 (m, 3 H), 4.85 (s, 4 H), 4.54 (br s, 2 H), 4.20 (s, 4 H), 3.99-4.05 (m, 1 H), 3.93 (br d, J = 6.2 Hz, 1 H), 3.64-3.77 (m, 5 H), 2.93-3.19 (m, 4 H), 2.67-2.75 (m, 2 H), 2.48 (br t, J = 5.1 Hz, 4 H), 2.35 (s, 3 H), 2.25 (br s, 2 H), 1.67 (br d, J = 13.1 Hz, 2 H). ¹⁹F NMR (376 MHz, MeOH- d4) δ (ppm) −76.94 (s, 3 F), −113.57-−111.71 (m, 2 F) | Intermediates E1 and B9 were used; |
| 1-091 | 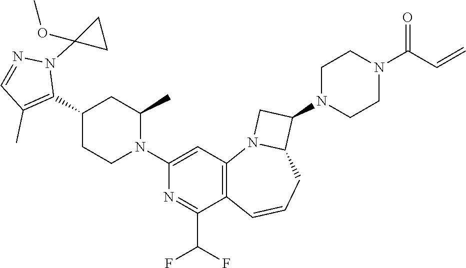 [SMILES: C=CC(=O)N1CCN([C@@H]2CN3c4cc(N5CC[C@H](c6c(C)cnn6C6(OC)CC6)C[C@H]5C)nc(C(F)F)c4C=CC[C@@H]23)CC1] 1-(4-((7aS,8R)-4-(difluoromethyl)-2- ((2R,4S)-4-(1-(1- methoxycyclopropyl)-4-methyl-1H- pyrazol-5-yl)-2-methyl-1-piperidinyl)- 7,7a,8,9-tetrahydroazeto[1,2- a]pyrido[3,4-f]azepin-8-yl)-1- piperazinyl)-2-propen-1-one | m/z (ESI): 608.1 (M + H)⁺. ¹H NMR (CDCl₃, 400 MHz) δ (ppm) 7.20 (s, 1 H), 6.85 (br d, J = 12.1 Hz, 1 H), 6.40-6.72 (m, 2 H), 6.30 (dd, J = 16.8, 1.8 Hz, 1 H), 5.67-5.76 (m, 2 H), 5.56 (s, 1 H), 4.73-4.86 (m, 1 H), 4.32 (br d, J = 12.5 Hz, 1 H), 3.93-4.02 (m, 2 H), 3.52- 3.84 (m, 6 H), 3.21 (s, 3 H), 2.96-3.03 (m, 1 H). 2.96- 3.12 (m, 1 H), 2.68-2.79 (m, 1 H), 2.57-2.67 (m, 1 H), 2.44 (br s, 4 H), 2.16-2.25 (m, 1 H), 2.09 (s, 3 H), 1.96- 2.08 (m, 2 H), 1.87 (br d, J = 12.3 Hz, 1 H), 1.72 (br d, J = 13.2 Hz, 1 H), 1.31-1.43 (m, 4 H), 1.25 (d, J = 6.9 Hz, 3 H). ¹⁹F NMR (CDCl₃, 376 MHz) δ (ppm) −114.6-−108.0 (m, 2F) | Intermediates E1 and B8 were used; |
| 1-092 | 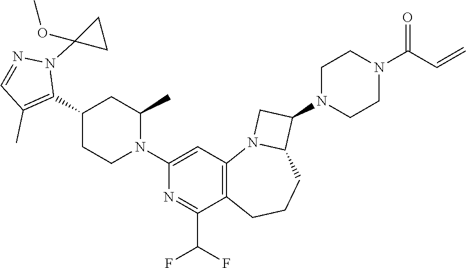 [SMILES: C=CC(=O)N1CCN([C@@H]2CN3c4cc(N5CC[C@H](c6c(C)cnn6C6(OC)CC6)C[C@H]5C)nc(C(F)F)c4CCC[C@@H]23)CC1] 1-(4-((7aS,8R)-4-(difluoromethyl)-2- ((2R,4S)-4-(1-(1- methoxycyclopropyl)-4-methyl-1H- pyrazol-5-yl)-2-methyl-1-piperidinyl)- 5,6,7,7a, 8,9-hexahydroazeto[1,2- a]pyrido[3,4-f]azepin-8-yl)-1- piperazinyl)-2-propen-1-one | m/z (ESI): 610.2 (M + H)⁺. ¹H NMR (CDCl₃, 400 MHz) δ (ppm) 7.21 (s, 1 H), 6.53- 6.62 (m, 1 H), 6.52 (br t, J = 55.4 Hz, 1 H), 6.31 (br d, J = 16.9 Hz, 1 H), 5.69-5.78 (m, 2 H), 4.78 (br s, 1 H), 4.30 (br d, J = 12.3 Hz, 1 H), 3.55- 3.87 (m, 8 H), 3.22 (s, 2 H), 3.16-3.28 (m, 1 H), 3.06 (br t, J = 12.5 Hz, 1 H), 2.97 (q, J = 6.0 Hz, 1 H), 2.35-2.52 (m, 5 H), 2.22 (td, J = 12.8, 4.9 Hz, 1 H), 2.10 (s, 3 H), 1.80-2.07 (m, 5 H), 1.72 (br d, J = 12.3 Hz, 1 H), 1.33-1.51 (m, 6 H), 1.25 (br d, J = 6.7 Hz, 3 H). 19 F NMR (CDCl₃, 376 MHz) δ (ppm) −110.63 (br d, 2F, J = 51.2 Hz)s | Intermediates E1 and B8 were used; |
| 1-093 | 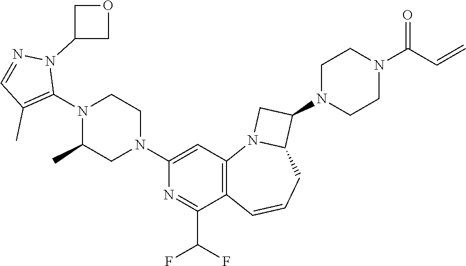 [SMILES: C=CC(=O)N1CCN([C@@H]2CN3c4cc(N5CCN(c6c(C)cnn6C6COC6)[C@H](C)C5)nc(C(F)F)c4C=CC[C@@H]23)CC1] 1-(4-((7aS,8R)-4-(difluoromethyl)-2- ((3R)-3-methyl-4-(4-methyl-1-(3- oxetanyl)-1H-pyrazol-5-yl)-1- piperazinyl)-7,7a,8,9- tetrahydroazeto[1,2-a]pyrido[3,4- f]azepin-8-yl)-1-piperazinyl)-2- propen-1-one | m/z (ESI): 595.2 (M + H)+. ¹H NMR (CDCl₃, 500 MHz) δ (ppm) 7.35-7.38 (m, 1 H), 6.85 (br d, J = 12.3 Hz, 1 H), 6.44-6.67 (m, 2 H), 6.29- 6.34 (m, 1 H), 5.74-5.81 (m, 2 H), 5.71-5.74 (m, 1 H), 5.58 (s, 1 H), 5.15 (dt, J = 19.1, 6.5 Hz, 2 H), 4.89-4.96 (m, 2 H), 4.12-4.33 (m, 2 H), 3.95- 4.00 (m, 2 H), 3.49-3.83 (m, 6 H), 3.25-3.36 (m, 2 H), 2.95-3.06 (m, 2 H), 2.90 (br d, J = 10.1 Hz, 1 H), 2.70-2.77 (m, 1 H), 2.60-2.68 (m, 2 H), 2.43 (br dd, J = 9.4, 4.7 Hz, 5 H), 2.05-2.07 (m, 3 H), 0.83 (d, J = 6.2 Hz, 3 H). ¹⁹F NMR (CDCl₃, 471 MHz) δ (ppm) −116.7-−102.6 (m, 2 F) | Intermediates E1 and C3 were used; |
| 1-094 | 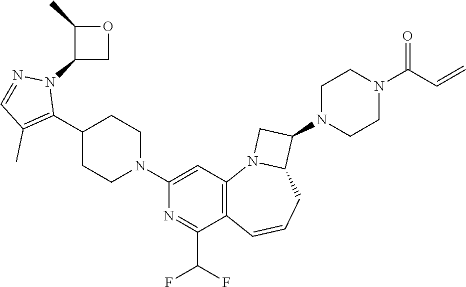 [SMILES: C=CC(=O)N1CCN([C@@H]2CN3c4cc(N5CCC(c6c(C)cnn6[C@@H]6CO[C@@H]6C)CC5)nc(C(F)F)c4C=CC[C@@H]23)CC1] 1-(4-((7aS,8R)-4-(difluoromethyl)-2- (4-(4-methyl-1-((2R,3R)-2-methyl-3- oxetanyl)-1H-pyrazol-5-yl)-1- piperidinyl)-7,7a,8,9- tetrahydroazcto[1,2-a]pyrido[3,4- f]azepin-8-yl)-1-piperazinyl)-2- propen-1-one | m/z (ESI): 594.3 (M + H)+. ¹H NMR (CDCl₃, 500 MHz) δ (ppm) 7.35 (s, 1 H), 6.86 (br d, J = 12.6 Hz, 1 H), 6.44-6.69 (m, 2 H), 6.32 (dd, J = 16.8, 1.9 Hz, 1 H), 5.77 (ddd, J = 9.5, 6.6, 3.4 Hz, 1 H), 5.73 (dd, J = 10.5, 1.8 Hz, 1 H), 5.62 (s, 1 H), 5.41 (quin, J = 6.2 Hz, 1 H), 5.04-5.11 (m, 1 H), 4.95 (q, J = 7.1 Hz, 1 H), 4.77 (dd, J = 7.4, 6.1 Hz, 1 H), 4.49-4.55 (m, 1 H), 4.42-4.49 (m, 1 H), 3.98 (t, J = 6.9 Hz, 2 H), 3.55- 3.85 (m, 5 H), 3.01 (q, J = 6.7 Hz. 1 H), 2.60-2.89 (m, 5 H), 2.34-2.53 (m, 4 H), 2.09 (s, 3 H), 1.95-2.06 (m, 2 H), 1.71- 1.85 (m, 2 H), 1.54 (d, J = 6.2 Hz, 3 H) | Intermediates E1 and B31′ were used; |
| 1-095 | 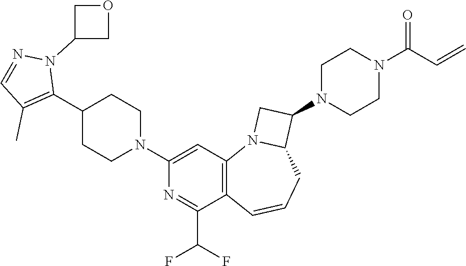 [SMILES: C=CC(=O)N1CCN([C@@H]2CN3c4cc(N5CCC(c6c(C)cnn6C6COC6)CC5)nc(C(F)F)c4C=CC[C@@H]23)CC1] 1-(4-((7aS,8R)-4-(difluoromethyl)-2- (4-(4-methyl-1-(3-oxetanyl)-1H- pyrazol-5-yl)-1-piperidinyl)-7,7a,8,9- tetrahydroazeto[1,2-a]pyrido[3,4- f]azepin-8-yl)-1-piperazinyl)-2- propen-1-one | m/z (ESI): 580.0 (M + H)⁺. ¹H NMR (400 MHz, MeOH- d4) δ (ppm) 7.34 (s, 1 H), 6.73- 6.82 (m, 2 H), 6.42-6.71 (m, 1 H), 6.22 (dd, J = 16.8, 1.9 Hz, 1 H), 5.76-5.80 (m, 2 H), 5.67-5.76 (m, 2 H), 5.06- 5.12 (m, 2 H), 4.97-5.04 (m, 2 H), 4.44-4.55 (m, 2 H), 4.01 (t. J = 7.6 Hz, 1 H), 3.89- 3.96 (m, 1 H), 3.65-3.76 (m, 5 H), 2.97-3.08 (m, 2 H), 2.87-2.95 (m. 2 H), 2.71 (br t, J = 4.8 Hz, 2 H), 2.48 (br t, J = 5.1 Hz, 4 H), 2.08 (s, 3 H), 1.89-2.04 (m, 2 H), 1.74 (br d, J = 11.0 Hz, 2 H). | Intermediates E1 and B23 were used; |
| 1-096 | 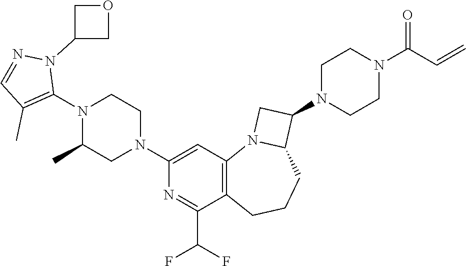 [SMILES: C=CC(=O)N1CCN([C@@H]2CN3c4cc(N5CCN(c6c(C)cnn6C6COC6)[C@H](C)C5)nc(C(F)F)c4CCC[C@@H]23)CC1] 1-(4-((7aS,8R)-4-(difluoromethyl)-2- ((3R)-3-methyl-4-(4-methyl-1-(3- oxetanyl)-1H-pyrazol-5-yl)-1- piperazinyl)-5,6,7,7a,8,9- hexahydroazeto[1,2-a]pyrido[3,4- f]azepin-8-yl)-1-piperazinyl)-2- propen-1-one | m/z (ESI): 597.25 (M + H)⁺. ¹H NMR (CDCl₃, 500 MHz) δ 7.36 (s, 1 H), 6.35-6.65 (m, 2 H), 6.30 (dd, J = 16.7, 1.9 Hz, 1 H), 5.69-5.81 (m, 3 H), 5.15 (dt, J = 13.3, 6.5 Hz, 2 H), 4.92 (dt, J = 7.8, 5.9 Hz, 2 H), 4.17 (br dd, J = 34.4, 12.0 Hz, 2 H), 3.60-3.84 (m, 6 H), 3.13- 3.38 (m, 3 H), 2.85-3.06 (m, 3 H), 2.54-2.66 (m, 1 H), 2.35-2.52 (m, 5 H), 2.06 (s, 3 H), 1.78-2.03 (m, 4 H), 1.39- 1.53 (m, 1 H), 0.83 (d, J = 6.3 Hz, 3 H). ¹⁹F NMR (CDCl₃, 471 MHz) δ (ppm) −114.2-−104.4 (m, 2F) | Intermediates E1 and C1 were used; |
| 1-103 | 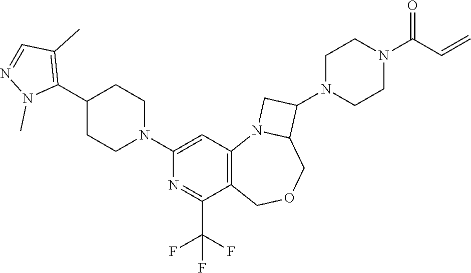 [SMILES: C=CC(=O)N1CCN(C2CN3c4cc(N5CCC(c6c(C)cnn6C)CC5)nc(C(F)(F)F)c4COCC23)CC1] 1-(4-(2-(4-(1,4-dimethyl-1H-pyrazol- 5-yl)piperidin-1-yl)-4- (trifluoromethyl)-7,7a,8,9-tetrahydro- 5H-azeto[2,1-c]pyrido[4,3- e][1,4]oxazepin-8-yl)piperazin-1- yl)prop-2-en-1-one | m/z (ESI): 559.9 (M + H)⁺. ¹H NMR (400 MHz, MeOH-d₄) δ (ppm) 7.15 (1 H, s) 6.76 (1 H, dd, J = 16.93, 10.66 Hz) 6.21 (1 H, dd, J = 16.83, 1.78 Hz) 6.11 (1 H, s) 5.76 (1 H, dd, J = 10.56, 1.78 Hz) 5.09 (1 H, d, J = 14.20 Hz) 4.58 (2 H, brt, J = 15.36 Hz) 4.21 (1 H, d, J = 14.21 Hz) 4.07 (1 H, dd, J = 11.29, 2.51 Hz) 3.76-3.89 (6 H, m) 3.59-3.72 (5 H, m) 3.08-3.21 (2 H, m) 2.91- 3.01 (2 H, m) 2.39-2.51 (4 H, m) 2.06 (3 H, s) 1.95-2.04 (2 H, m) 1.84 (2 H, br d, J = 12.33 Hz). | Intermediates E13 and B20 were used; Mixture of trans isomers. |
| 1-104 | 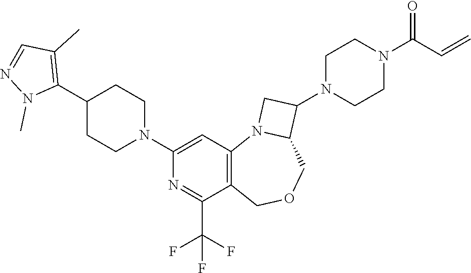 [SMILES: C=CC(=O)N1CCN(C2CN3c4cc(N5CCC(c6c(C)cnn6C)CC5)nc(C(F)(F)F)c4COC[C@@H]23)CC1] 1-(4-((7aR,8R)-2-(4-(1,4-dimethyl-1H- pyrazol-5-yl)-1-piperidinyl)-4- (trifluoromethyl)-7,7a,8,9-tetrahydro- 5H-azeto[2,1-c]pyrido[4,3- e][1,4]oxazepin-8-yl)-1-piperaziny])-2- propen-1-one | m/z (ESI): 560.2 (M + H)⁺. ¹H NMR (600 MHz, DMSO-d₆) δ (ppm) 7.07 (1 H, s) 6.79 (1 H, dd, J = 16.65, 10.49 Hz) 6.14 (1 H, s) 6.10 (1 H, dd, J = 16.58, 2.35 Hz) 5.68 (1 H, dd, J = 10.42, 2.35 Hz) 4.91 (1 H, d, J = 14.10 Hz) 4.49 (2 H, t, J = 14.00 Hz) 4.14 (1 H, d, J = 14.23 Hz) 3.98 (1 H, dd, J = 11.30, 2.49 Hz) 3.75-3.84 (6 H, m) 3.49-3.60 (4 H, m), 3.25-3.30 (m, 2 H), 3.01- 3.11 (2 H, m) 2.82-2.90 (2 H, m) 2.26-2.36 (4 H, m) 1.98 (3 H, s) 1.73-1.85 (4 H, m). | Intermediates E13 and B20 were used; Purified via SFC using a ChiralPak ID, 2 × 25 cm 5 μm column with a mobile phase of 45% MeOH using a flowrate of 80 mL/min (1st eluting isomer) |
| 1-109 | 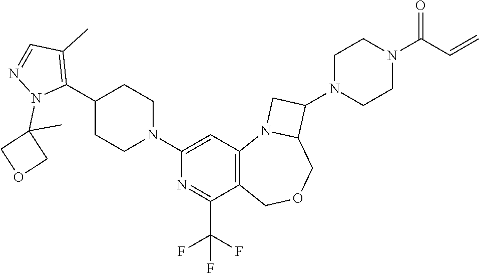 [SMILES: C=CC(=O)N1CCN(C2CN3c4cc(N5CCC(c6c(C)cnn6C6(C)COC6)CC5)nc(C(F)(F)F)c4COCC23)CC1] 1-(4-(2-(4-(4-methyl-1-(3- methyloxetan-3-yl)-1H-pyrazol-5- yl)piperidin-1-yl)-4-(trifluoromethyl)- 7,7a,8,9-tetrahydro-5H-azeto[2,1- c]pyrido[4,3-c][1,4]oxazepin-8- yl)piperazin-1-yl)prop-2-en-1-one | m/z (ESI): 598.0 (M + H)⁺. ¹H NMR (400 MHz, (CDCl₃) δ (ppm) 7.21-7.25 (m, 1 H), 6.35-6.66 (m, 2 H), 6.30 (dd, J = 16.9, 1.9 Hz, 1 H), 5.83 (s, 1 H), 5.68-5.75 (m, 1 H), 5.32 (br d, J = 13.6 Hz, 1 H), 5.26 (d, J = 6.3 Hz, 2 H), 4.63-4.68 (m, 2 H), 4.42-4.55 (m, 2 H), 4.16-4.23 (m, 1 H), 4.00- 4.06 (m, 1 H), 3.68-3.86 (m, 4 H), 3.47-3.66 (m, 4 H), 3.09 (g, J = 6.9 Hz, 1 H), 2.70- 2.82 (m, 2 H), 2.30-2.52 (m, 4 H), 2.21-2.29 (m, 1 H), 2.01-2.12 (m, 5 H), 1.82- 1.86 (m, 3 H), 1.78 (br s, 2 H). ¹⁹F NMR (CDCl₃, 471 MHz) δ (ppm) −112.1-−109.7 (m, 2 F) | Intermediate E13 and B6 were used; Mixture of trans isomers. |

### Method 6

### Example 97:1-(4-((7aS,8R)-2-(4-(1-(2-Methoxyethyl)-4-methyl-1H-pyrazol-5-yl)-1-piperidinyl)-4-(trifluoromethyl)-7,7a,8,9-tetrahydroazeto[1,2-a]pyrido[3,4-f]azepin-8-yl)-]-piperazinyl)-2-propen-1-one

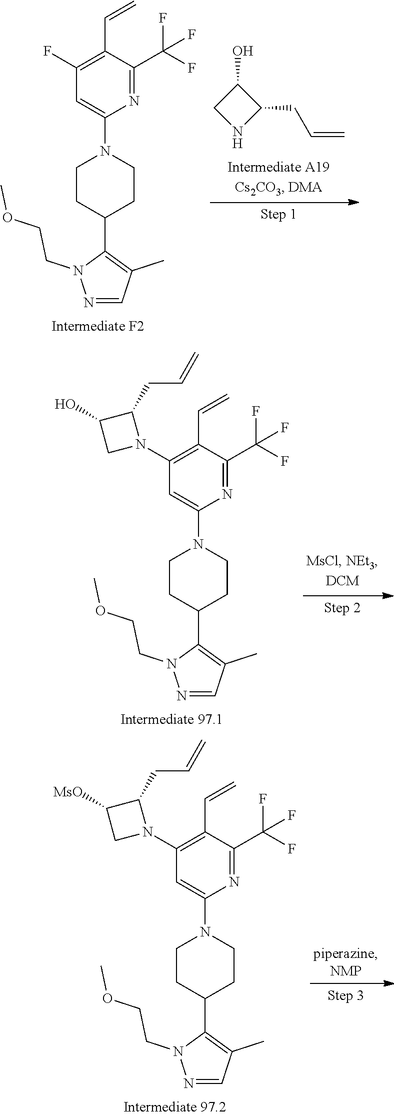

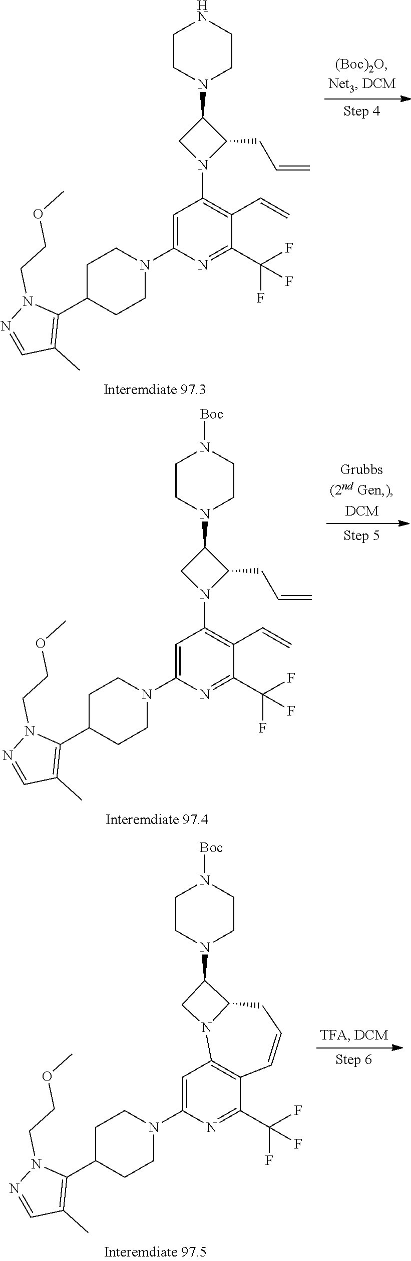 [SMILES: C=CC[C@H]1[C@H](N2CCNCC2)CN1c1cc(N2CCC(c3c(C)cnn3CCOC)CC2)nc(C(F)(F)F)c1C=C ; C=CC[C@H]1[C@H](N2CCN(C(=O)OC(C)(C)C)CC2)CN1c1cc(N2CCC(c3c(C)cnn3CCOC)CC2)nc(C(F)(F)F)c1C=C ; COCCn1ncc(C)c1C1CCN(c2cc3c(c(C(F)(F)F)n2)C=CC[C@H]2[C@H](N4CCN(C(=O)OC(C)(C)C)CC4)CN32)CC1]

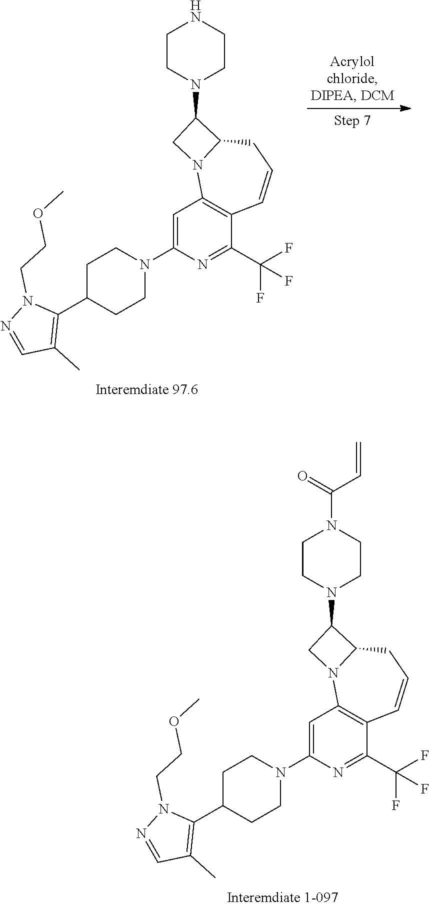 [SMILES: COCCn1ncc(C)c1C1CCN(c2cc3c(c(C(F)(F)F)n2)C=CC[C@H]2[C@H](N4CCNCC4)CN32)CC1 ; C=CC(=O)N1CCN([C@@H]2CN3c4cc(N5CCC(c6c(C)cnn6CCOC)CC5)nc(C(F)(F)F)c4C=CC[C@@H]23)CC1]

Step 1. (25,3S)-2-Allyl-1-(6-(4-(1-(2-methoxyethyl)-4-methyl-1H-pyrazol-5-yl)piperidin-1-yl)-2-(trifluoromethyl)-3-vinylpyridin-4-yl)azetidin-3-ol, Intermediate 97.1. To a stirred solution of Intermediate F2 (2.0 g, 4.85 mmol) in DMA (8.0 mL) was added Cs₂CO₃ (4.74 g, 14.55 mmol) and Intermediate A19 (1.427 g, 6.79 mmol). The resulting reaction mixture was stirred for 16 h at 90° C. After, the reaction mixture was diluted with ice cold water and extracted with EtOAc (2×30 mL). The combined organic extracts were washed with brine (30 mL), dried over Na₂SO₄, filtered, and concentrated. The crude material was purified by chromatography, eluting with a gradient of 50-80% EtOAc in pet. ether to give Intermediate 97.1 (1.2 g, 2.37 mmol, 49% yield), m/z (ESI): 506.1 (M+H)⁺.

Step 2. (2S,3S)-2-Allyl-1-(6-(4-(1-(2-methoxyethyl)-4-methyl-1H-pyrazol-5-yl)piperidin-1-yl)-2-(trifluoromethyl)-3-vinylpyridin-4-yl)azetidin-3-yl methanesulfonate, Intermediate 97.2. To a solution of Intermediate 97.1 (1.2 g, 2.37 mmol) in DCM at 0° C. was added Et₃N (0.331 mL, 2.37 mmol) followed by MsCl (0.185 mL, 2.37 mmol) dropwise. The reaction mixture was stirred at 0° C. for 1 h. The volatiles were removed under vacuum, and the crude was purified by chromatography, eluting with a gradient of 60-80% EtOAc in pet. ether to provide Intermediate 97.2 (1.2 g, 2,056 mmol, 87% yield), m/z (ESI): 584.1 (M+H)⁺.

Step 3. 1-((2S,3R)-2-Allyl-1-(6-(4-(1-(2-methoxyethyl)-4-methyl-1H-pyrazol-5-yl)piperidin-1-yl)-2-(trifluoromethyl)-3-vinylpyridin-4-yl)azetidin-3-yl)piperazine, Intermediate 97.3. To a solution of Intermediate 97.2 (0.813 g, 1.393 mmol) in NMP (12 mL), was added piperazine (1.2 g, 13.93 mmol). The reaction mixture was heated to 120° C. for 40 h, cooled to rt, water added to it, and extracted with EtOAc (20 mL×3). The combined organic extracts were washed with water, brine, dried over Na₂SO₄ and concentrated to provide Intermediate 97.3 (0.9 g). The crude product was used for the next step without further purification, m/z (ESI): 574.2 (M+H)⁺

Step 4. tert-Butyl 4-((2S,3R)-2-allyl-1-(6-(4-(1-(2-methoxyethyl)-4-methyl-1H-pyrazol-5-yl)piperidin-1-yl)-2-(trifluoromethyl)-3-vinylpyridin-4-yl)azetidin-3-yl)piperazine-1-carboxylate, Intermediate 97.4. To a stirred solution of Intermediate 97.3 (0.9 g, 1.57 mmol) in DCM (50 mL) at 0° C. was dropwise added Et₃N (0.656 mL, 4.71 mmol), and the mixture stirred for 5 min. Then, a solution of Boc-anhydride (0.728 mL, 3.14 mmol) in DCM (10 mL) was added dropwise at 0° C., and the reaction stirred at rt for 16 h. After, water was added to the reaction mixture, and the mixture was extracted with DCM (20 mL×3). The organic extracts were combined, dried over Na₂SO₄ and concentrated. The crude material was purified by column chromatography to provide Intermediate 97.4 (0.30 g, 0.245 mmol, 16% yield) and tert-butyl 4-((28,3S)-2-allyl-1-(6-(4-(1-(2-methoxyethyl)-4-methyl-1H-pyrazol-5-yl)piperidin-1-yl)-2-(trifluoromethyl)-3-vinylpyridin-4-yl)azetidin-3-yl)piperazine-1-carboxylate (0.25 g, 0.371 mmol, 24% yield), m/z (ESI): 674.3 (M+H)⁺.

Step 5. tert-Butyl 4-((7aS,8R)-2-(4-(1-(2-methoxyethyl)-4-methyl-1H-pyrazol-5-yl)piperidin-1-yl)-4-(trifluoromethyl)-7,7a,8,9-tetrahydroazeto[1,2-a]pyrido[3,4-f]azepin-8-yl)piperazine-1-carboxylate, Intermediate 97.5. To a solution of Intermediate 97.4 (0.25 g, 0.371 mmol) in DCM (7.50 mL) at rt was added Grubbs II catalyst (0.031 g, 0.037 mmol), and the reaction mixture was heated at reflux and stirred for 16 b. Another portion of Grubbs II catalyst (20 mg) was added to the reaction mixture and stirred for another 24 h. The volatiles were evaporated, and the mixture was filtered, dried and purified by prep-HPLC (YMC, 0.1% NH₃ in water/ACN, 15 mL/min flow rate) to provide Intermediate 97.5 (140 mg, 0.217 mmol, 58% yield), m/z (ESI): 646.3 (M+H)⁺. ¹H NMR (400 MHz, DMSO-d₆) δ (ppm) 7.12 (s, 1H), 6.46 (d, J=12.8 Hz, 1H), 5.87 (s, 1H), 5.81-5.76 (m, 1H), 4.45 (t, J=12.8 Hz, 2H), 4.22 (t, J=5.2 Hz, 2H), 3.93 (t, J=7.2 Hz, 1H), 3.80 (t, J=6.4 Hz, 1H), 3.70 (t, J=7.2 Hz, 1H), 3.61 (t, J=5.2 Hz, 2H), 3.21 (s, 3H), 3.10-3.06 (m, 1H), 2.98-2.92 (m, 1H), 2.86-2.80 (m, 2H), 2.68-2.63 (m, 2H), 2.33-2.25 (m, 4H), 2.08 (s, 3H), 1.98 (s, 3H), 1.81-1.73 (m, 4H), 1.40 (s, 9H).

Step 6: (7aS,8R)-2-(4-(1-(2-Methoxyethyl)-4-methyl-1H-pyrazol-5-yl)piperidin-1-yl)-8-(piperazin-1-yl)-4-(trifluoromethyl)-7,7a,8,9-tetrahydroazeto[1,2-a]pyrido[3,4-f]azepine trifluoroacetate, Intermediate 97.6. To a solution of Intermediate 97.5 (50 mg, 0.077 mmol) in DCM (5 mL) was added TFA (0.05 mL) at 0° C., and the reaction mixture was stirred at rt for 2 h. The solvent was concentrated to afford Intermediate 97.6 (50 mg crude). The crude TFA salt was taken for next step without further purification, m/z (ESI): 546.3 (M+H)⁺.

Step 7:1-(4-((7aS,8S)-2-(4-(1-(2-Methoxyethyl)-4-methyl-1H-pyrazol-5-yl)piperidin-1-yl)-4-(trifluoromethyl)-7,7a,8,9-tetrahydroazeto[1,2-a]pyrido[3,4-f]azepin-8-yl)piperazin-1-yl)prop-2-en-1-one, Compound 1-097. To a stirred solution of Intermediate 97.6 (50 mg, 0.078 mmol) in DCM (5 mL) at −78° C., was added DIPEA (50 mg, 0.389 mmol) followed by a solution of acryloyl chloride (95 μL, 0.086 mmol) in DCM (0.5 mL) dropwise. The reaction mixture was stirred at −78° C. for 5 min. After, the reaction mixture was quenched with water and extracted with DCM (2×20 mL). The combined organic extracts were washed with water (15 mL), brine (15 mL), dried over anhydrous Na₂SO₄, filtered, and concentrated. The crude product was purified through prep-HPLC (YMC, 0.1% NH; in water/ACN, flow rate 15 mL/min) to provide 1-(4-((7aS,8R)-2-(4-(1-(2-methoxyethyl)-4-methyl-1H-pyrazol-5-yl)-1-piperidinyl)-4-(trifluoromethyl)-7,7a,8,9-tetrahydroazeto[1,2-a]pyrido[3,4-f]azepin-8-yl)-1-piperazinyl)-2-propen-1-one, Compound 1-097 (26 mg, 0.043 mmol, 56% yield), m/z (ESI): 600.3 (M+H)⁺. ¹H NMR (400 MHz, DMSO-d₆) δ (ppm) 7.13 (s, 1H), 6.81 (dd, J=16.7, 10.5 Hz, 1H), 6.47 (d./=12.03 Hz, 1H), 6.11 (d,)=16.4 Hz, 1H), 5.88 (s, 1H), 5.82-5.77 (m, 1H), 5.69 (d, J=10.02 Hz, 1H), 4.45 (t, J=11.6 Hz, 2H), 4.23 (t, J=5.4 Hz, 2H), 3.95 (br s, 1H), 3.85 (br s, 1H). 3.74 (br s, 1H), 3.61 (t, J=5.4 Hz, 2H), 3.55 (s, 4H), 3.21 (s, 3H), 3.07-3.04 (m, 2H), 2.85 (t, J=12.1 Hz, 2H), 2.66 (s, 2H), 2.34-2.33 (s, 3H), 1.98 (s, 3H), 1.84-1.77 (m, 4H).

Compounds in Table 2-6 were prepared following the procedure described in Method 6, using appropriate starting materials. All starting materials are commercially available or are described in the Intermediates section above.

TABLE 2-6

| Compound | | LCMS: (ESI + ve ion) m/z; | |
|---|---|---|---|
| # | Chemical Structure & Name | NMR | Comments |
| 1-098 | 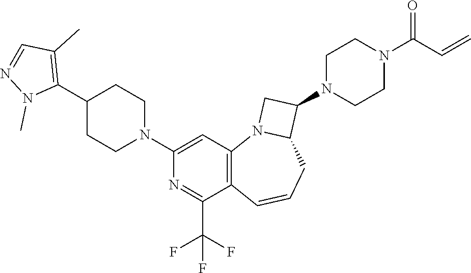 1-(4-((7aS,8R)-2-(4-(1,4-dimethyl-1H- pyrazol-5-yl)-1-piperidinyl)-4- (trifluoromethyl)-7,7a,8,9- tetrahydroazeto[1,2-a]pyrido[3,4- f]azepin-8-yl)-1-piperazinyl)-2-propen- 1-one | m/z (ESI): 656.3 (M + H)⁺. ¹H NMR (400 MHz, DMSO- d₆) δ (ppm) 7.08 (s, 1H), 6.80 (dd, J = 16.7, 10.5 Hz, 1H), 6.52-6.39 (m, 1H), 6.11 (dd, J = 16.7, 2.4 Hz, 1H), 5.88 (s, 1H), 5.79 (dt, J = 12.6, 4.8 Hz, 1H), 5.68 (dd, J = 10.5, 2.4 Hz, 1H), 4.47 (t, J = 12.0 Hz, 2H), 3.95 (t, J = 7.5 Hz, 1H), 3.85 (q, J = 6.1 Hz, 1H), 3.79- 3.69 (m, 4H), 3.64-3.46 (m, 4H), 3.13-2.76 (m, 4H), 2.66 (q, J = 5.6 Hz, 2H), 2.43- 2.27 (m, 4H), 1.99 (s, 3H), 1.79 (dq, J = 9.2, 5.3, 4.5 Hz, 4H). | Step 1. Intermediates F3 and A19 were used |
| 1-099 | 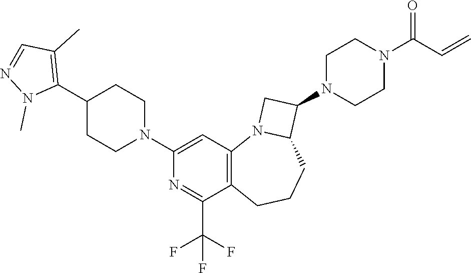 1-(4-((7aS,8R)-2-(4-(1,4-dimethyl-1H- pyrazol-5-yl)-1-piperidinyl)-4- (trifluoromethyl)-5,6,7,7a,8,9- hexahydroazeto[1,2-a]pyrido[3,4- f]azepin-8-yl)-1-piperazinyl)-2-propen- 1-one | m/z (ESI): 596.4 (M + H)⁺. ¹H NMR (400 MHz, MeOH- d4) δ (ppm) 7.07 (s, 1H), 6.81 (dd, J = 16.7, 10.4 Hz, 1H), 6.11 (dd, J = 16.7, 2.5 Hz, 1H), 6.00 (s, 1H), 5.68 (dd, J = 10.4, 2.4 Hz, 1H), 4.51-4.34 (m, 2H), 3.87-3.67 (m, 6H), 3.63-3.46 (m, 4H), 3.12- 2.72 (m, 5H), 2.34 (d, J = 7.1 Hz, 5H), 1.99 (s, 3H), 1.95- 1.69 (m, 7H), 1.47 (br s, 1 H); ¹⁹F NMR (376 MHz, MeOH- d4) δ (ppm) −112.44 (d, J = 21.7 Hz, 1 F). | Step 2. Intermediates F3 and A19 were used; The chiral center was arbitrarily assigned; Additional Step: Before Step 6: Intermediate F3 was reacted with palladium on activated carbon, Pd(OH)₂ in MeOH:THF |
| 1-100 | 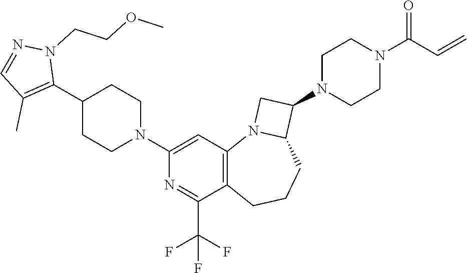 [SMILES: C=CC(=O)N1CCN([C@@H]2CN3c4cc(N5CCC(c6c(C)cnn6CCOC)CC5)nc(C(F)(F)F)c4CCC[C@@H]23)CC1] 1-(4-((7aS,8R)-2-(4-(1-(2- methoxyethyl)-4-methyl-1H-pyrazol-5- yl)-1-piperidinyl)-4-(trifluoromethyl)- 5,6,7,7a,8,9-hexahydroazeto[1,2- a]pyrido[3,4-f]azepin-8-yl)-1- piperazinyl)-2-propen-1-one | m/z (ESI): 602.1 (M + H)⁺. ¹H NMR (400 MHz, DMSO- d₆) δ (ppm) 7.12 (s, 1H), 6.84- 6.77 (m, 1H), 6.10 (dd, J = 16.4, 2.4 Hz, 1H), 6.00 (s, 1H), 5.68 (dd, J = 10.4, 2.4 Hz, 1H), 4.41-4.40 (m, 2H), 4.22 (t, J = 5.2 Hz, 2H), 3.82-3.73 (m, 3H), 3.62-3.54 (m, 6H), 3.21 (s, 3H), 3.07-3.03 (m, 1H), 2.92-2.87 (m, 2H), 2.81- 2.75 (m, 2H), 2.40-2.33 (m, 5H), 1.98 (s, 3H), 1.87-1.73 (m, 7H), 1.47-1.44 (m, 1H). | Step 2. Intermediates F2 and A19 were used: Additional Step: Before Step 6: Intermediate 97.5 was reacted with palladium on activated carbon, Pd(OH)₂ in MeOH:THF |

### Method 7

### Example 101:1-(4-((7aS,8R)-2-(4-(1-(2-methoxyethyl)-4-methyl-1H-pyrazol-5-yl)-1-piperidinyl)-4-(trifluoromethyl)-7,7a,8,9-tetrahydroazeto[1,2-a]pyrimido[5,4-f]azepin-8-yl)-1-piperazinyl)-2-propen-1-one

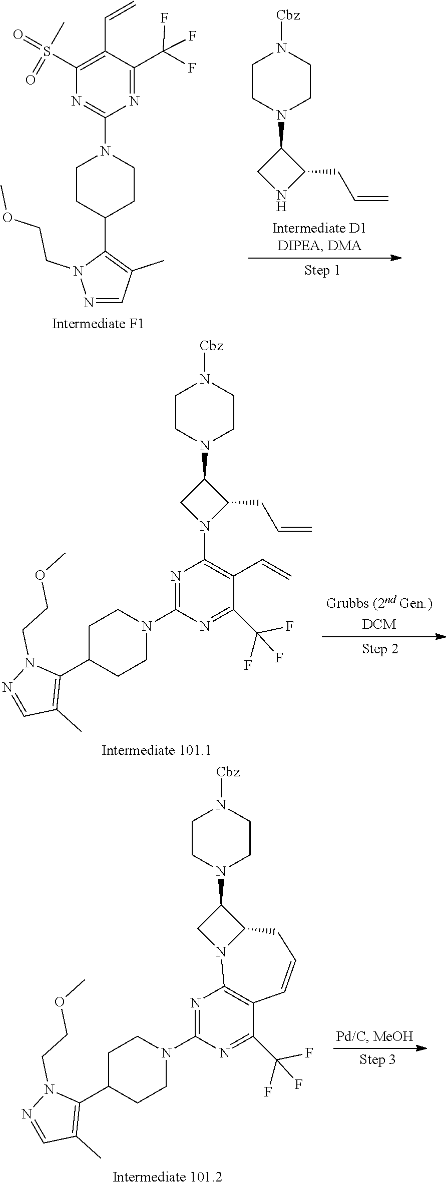 [SMILES: C=Cc1c(C(F)(F)F)nc(N2CCC(c3c(C)cnn3CCOC)CC2)nc1S(C)(=O)=O ; C=CC[C@@H]1NC[C@H]1N1CCN(C(=O)OCc2ccccc2)CC1 ; C=CC[C@H]1[C@H](N2CCN(C(=O)OCc3ccccc3)CC2)CN1c1nc(N2CCC(c3c(C)cnn3CCOC)CC2)nc(C(F)(F)F)c1C=C ; COCCn1ncc(C)c1C1CCN(c2nc3c(c(C(F)(F)F)n2)C=CC[C@H]2[C@H](N4CCN(C(=O)OCc5ccccc5)CC4)CN32)CC1]

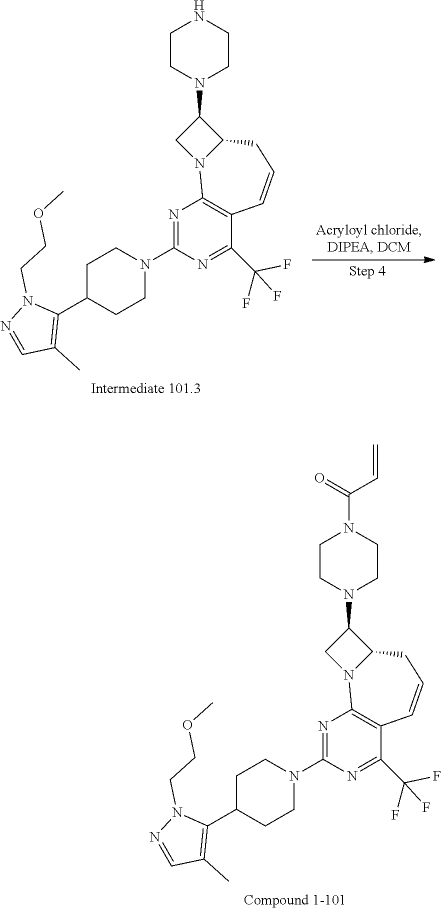 [SMILES: COCCn1ncc(C)c1C1CCN(c2nc3c(c(C(F)(F)F)n2)C=CC[C@H]2[C@H](N4CCNCC4)CN32)CC1 ; C=CC(=O)N1CCN([C@@H]2CN3c4nc(N5CCC(c6c(C)cnn6CCOC)CC5)nc(C(F)(F)F)c4C=CC[C@@H]23)CC1]

Step 1. Benzyl 4-((28,3R)-2-allyl-1-(2-(4-(1-(2-methoxyethyl)-4-methyl-1H-pyrazol-5-yl)piperidin-1-yl)-6-(trifluoromethyl)-5-vinylpyrimidin-4-yl)azetidin-3-yl)piperazine-1-carboxylate, Intermediate 101.1. To a solution of Intermediate F1 (0.6 g, 1.267 mmol) and Intermediate D1 (1.088 g, 2.53 mmol) in DMA (6.00 mL), was added DIPEA (4.43 mL. 25.3 mmol), and the reaction mixture was heated at 90° C. for 16 h. After, the reaction mixture was diluted with EtOAc (30 mL) and washed with brine (4×30 mL). The organic solution was dried over Na₂SO₄, filtered, and concentrated. The crude was purified by column chromatography, eluting with a gradient of 25-35% EtOAc/Pet ether to provide Intermediate 101.1 (0.51 g, 0.719 mmol, 57% yield), m/z (ESI): 709.2 (M+H)⁺.

Step 2. Benzyl 4-((7aS,8R)-2-(4-(1-(2-methoxyethyl)-4-methyl-1H-pyrazol-5-yl)piperidin-1-yl)-4-(trifluoromethyl)-7,7a,8,9-tetrahydroazeto[1,2-a]pyrimido[5,4-f]azepin-8-yl)piperazine-1-carboxylate, Intermediate 101.2. A solution of Intermediate 101.1 (0.51 g, 0.719 mmol) in DCM (15.30 mL) was degassed under N₂ for 5 min, and Grubbs II catalyst (0.061 g, 0.072 mmol) was added. The reaction mixture was refluxed for 40 h at 45° C. The reaction was concentrated and purified by column chromatography, eluting with a gradient of 50% to 60% EtOAc in pet. ether, to provide Intermediate 101.2 (0.185 g, 0.272 mmol, 38% yield), m/z (ESI): 681.3 (M+H)+¹H NMR (400 MHz, CDCl₃) δ (ppm) 7.45-7.33 (m, 5H), 7.25 (s, 1H), 6.54 (d, J=12.3 Hz, 1H), 5.80-5.65 (m, 1H), 5.16 (s, 2H), 4.98 (d, J=13.1 Hz, 2H), 4.28 (t, J=5.5 Hz, 2H), 4.14-3.93 (m, 3H), 3.74 (t, J=5.5 Hz, 2H), 3.56 (s, 4H), 3.33 (s, 3H), 3.01 (p, J=6.9, 6.3 Hz, 2H), 2.85 (t, J=13.1 Hz, 2H), 2.66 (br s, 2H), 2.39 (s, 4H), 2.08 (s, 3H), 1.91 (1, J=12.6 Hz, 2H), 1.82 (d, J=12.8 Hz, 2H).

Step 3. (7aS,8R)-2-(4-(1-(2-Methoxyethyl)-4-methyl-1H-pyrazol-5-yl)piperidin-1-yl)-8-(piperazin-1-yl)-4-(trifluoromethyl)-7,7a,8,9-tetrahydroazeto[1,2-a]pyrimido[5,4-f]azepine, Intermediate 101.3. To a solution of Intermediate 101.2 (0.185 g, 0.272 mmol) in MeOH (4.63 mL) was added 10 wt % palladium on activated carbon (0.04 g, 0.376 mmol). The reaction mixture was then stirred under H₂ atmosphere (bladder pressure) for 3 h at rt. Then, the reaction mixture was filtered and concentrated to provide crude Intermediate 101.3 (140 mg). The crude was used for next step reaction without further purification, m/z (ESI): 549.3 (M+H)⁺. ¹H NMR (400 MHz, DMSO-d₆) δ (ppm) 7.12 (s, 1H), 4.85-4.69 (m, 2H), 4.35 (s, 1H), 4.27-4.20 (m, 2H), 4.08 (dt, J=33.9, 8.4 Hz, 2H), 3.91-3.67 (m, 2H), 3.61 (t, J=5.3 Hz, 2H), 3.21 (d, J=1.3 Hz, 3H), 3.14-2.77 (m, 5H), 2.70 (d, J=5.4 Hz, 6H), 2.25 (s, 3H), 1.98 (s, 3H), 1.81-1.42 (m, 6H).

Step 4. 1-(4-((7aS,8R)-2-(4-(1-(2-Methoxyethyl)-4-methyl-1H-pyrazol-5-yl)piperidin-1-yl)-4-(trifluoromethyl)-7,7a,8,9-tetrahydroazeto[1,2-a]pyrimido[5,4-f]azepin-8-yl)piperazin-1-yl)prop-2-en-1-one, Compound 1-101. To a solution of Intermediate 101.3 (0.070 g, 0.128 mmol) in DCM (1.4 mL) was added DIPEA (0.067 mL, 0.383 mmol) at −78° C. Then, a solution of acryloyl chloride (0.028 g, 0.306 mmol) in DCM (2.70 mL) was added to the reaction mixture dropwise, and the reaction mixture was stirred at −78° C. for 5 min. The reaction was quenched with ice-water (10 mL) and extracted with DCM (3×10 mL). The combined organic extracts were dried over Na₂SO₄, filtered, concentrated, and purified by prep-HPLC using a Kinetex EVO C-18 column (250×21.2) mm, 5.0 μm, phase A: 0.1% formic acid in water, B: -ACN as the mobile phase, and a flow rate of 15 mL/min, to provide Compound 1-101. 2^{nd} Eluting compound: m/z (EST): 601.3 (M+H)⁺. ¹H NMR (400 MHz, DMSO-d₆) δ (ppm) 7.13 (s, 1H), 6.81 (dd, J=16.7, 10.5 Hz, 1H), 6.40-6.33 (m, 1H), 6.11 (dd, J=16.7, 2.4 Hz, 1H), 5.82 (ddd, J=12.1, 7.3, 3.4 Hz, 1H), 5.68 (dd, J=10.5, 2.4 Hz, 1H), 4.81 (d, J=12.9 Hz, 2H), 4.23 (t, J=5.4 Hz, 2H), 4.19-4.04 (m, 2H), 3.90 (dd, J=9.5, 6.5 Hz, 1H), 3.71-3.43 (m, 6H), 3.22 (s, 3H), 3.02-3.17 (m, 2H), 2.82-2.95 (m, 2H), 2.62-2.73 (m, 2H) (overlapped with solvent signal) 2.35 (br s, 5H), 1.98 (s, 3H), 1.76 (s, 4H).

Compounds in Table 2-7 were prepared following the procedure described in Method 7, using appropriate starting materials. All starting materials are commercially available or are described in the Intermediates section above.

TABLE 2-7

| Compound | | LCMS: (ESI + ve ion) m/z; | |
|---|---|---|---|
| # | Chemical Structure & Name | NMR | Comments |
| 1-105 | 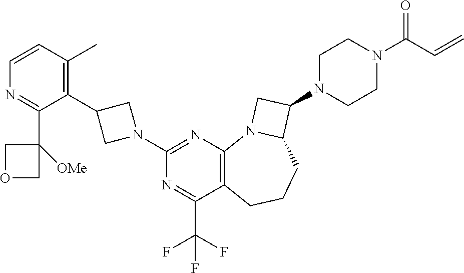 [SMILES: C=CC(=O)N1CCN([C@@H]2CN3c4nc(N5CC(c6c(C)ccnc6C6(OC)COC6)C5)nc(C(F)(F)F)c4CCC[C@@H]23)CC1] 1-(4-((7aS,8R)-2-(3-(2-(3- methoxyoxetan-3-yl)-4- methylpyridin-3-ylazetidin-1-yl)-4- (trifluoromethyl)-5,6,7,7a,8,9- hexahydroazeto[1,2- a]pyrimido[5,4-f]azepin-8- yl)piperazin-1-yl)prop-2-en-1-one | m/z (ESI): 596.4 (M + H)⁺; ¹H NMR (400 MHz, DMSO-d₆) δ ppm 8.33 (d, J = 4.9 Hz, 1H), 7.28 (d, J = 5.0 Hz, 1H), 6.4- 6.8 (m, 2H), 6.11 (dd, J = 16.7, 2.4 Hz, 1H), 5.68 (dd, J = 10.4, 2.4 Hz, 1H), 5.03 (dd, J = 7.2, 4.4 Hz, 2H), 4.79 (d, J = 7.2 Hz, 2H), 4.33 (td, J = 9.2, 5.2 Hz, 3H), 4.18 (dd, J = 9.0, 6.2 Hz, 2H), 4.10 (t, J = 8.8 Hz, 1H), 3.90 (ddd, J = 15.9, 9.6, 6.2 Hz, 1H), 3.75 (dd, J = 9.7, 5.9 Hz, 1H), 3.57 (d, J = 7.9 Hz, 4H), 2.94 (s, 3H), 2.83 (q, J = 6.2 Hz, 1H), 2.73 (dd, J = 15.0, 7.5 Hz, 1H), 2.65 (s, 3H), 2.55 (s, 1H), 2.32 (d, J = 5.3 Hz, 4H), 1.7- 1.9 (m, 2H), 1.72 (d, J = 5.1 Hz, 2H). | |

### Section 3: Biochemical and Cellular Assays

Provided in this section is the biological evaluation of the specific examples provided herein. See Table 3.

### Coupled Nucleotide Exchange Assay

The KRAS^{G12C} coupled nucleotide exchange assay allows for the screening and profiling of KRAS^{G12C} antagonists/inhibitors by monitoring the binding of an effector protein (e.g., a Ras binding domain of Raf1, RBD-cRaf) to KRAS^{G12C}. Purified GDP-bound KRAS protein (aa 1-169), containing both G12C and C118A amino acid substitutions and an N-terminal His-tag, was pre-incubated in assay buffer (25 mM HEPES pH 7.4, 10 mM MgCl₂, 0.01% Triton X-100, cither with or without 0.1% BSA) with serially diluted compound for 2 h. For all subsequent steps, BSA was omitted and DTT was added to the reaction buffer at a final concentration of 1 mM. Following compound pre-incubation, purified SOS protein (aa 564-1049) and GTP (Roche 10106399001) were added to the assay wells and incubated for an additional 30 min. To determine the extent of inhibition of SOS-mediated nucleotide exchange, purified GST-tagged cRAF (aa 1-149), nickel chelate AlphaLISA acceptor beads (PerkinElmer AL108R), and AlphaScreen glutathione donor beads (PerkinElmer 6765302) were added to the assay wells and incubated for 5-30 min. The assay plates were then read on a plate reader measuring luminescence signal. Signal intensity of compound-containing wells were normalized to DMSO control, and data were analyzed using a 4-parameter logistic model to calculate IC₅₀ values.

Other compounds disclosed herein can be prepared by analogous methods to the general methods and example above.

### Cell Viability Assay

MIA PaCa-2 (human pancreatic carcinoma; ATCC CRL-1420) or A549 (human lung carcinoma; ATCC CCL-185) cells were cultured in RPMI 1640 medium containing 10% fetal bovine serum and 1× penicillin/streptomycin/L-glutamine. Cells were seeded in 384-well plates at a density of 1.67E+04 cells/mL and incubated at 37° C., 5% CO₂, overnight. Serially-diluted compound or DMSO was added to the cells, and plates were incubated at 37° C., 5% CO₂ for 72 b. Cell viability was measured using a CellTiter-Glo® Luminescent Cell Viability Assay kit (Promega) according to the manufacturer's protocol. The luminescence signal of treated samples was normalized to DMSO control, and data were analyzed using a 4-parameter logistic model to calculate IC₅₀ values.

The following data (Table 3) categorizes the IC₅₀ of each compound for inhibiting enzymatic activity of KRAS^{G12C} in the indicated cells.

TABLE 3

| | Coupled Exchange | MIA PaCa-2, IC₅₀ | A549, IC₅₀ |
|---|---|---|---|
| Compound # | IC₅₀ (μM) | (μM) | (μM) |
| 1-001 | 0.014 | 0.017 | >5.0 |
| 1-002 | 0.009 | 0.021 | >5.0 |
| 1-003 | 0.013 | 0.013 | >5.0 |
| 1-004 | 0.016 | 0.023 | >5.0 |
| 1-005 | 0.025 | 0.017 | >5.0 |
| 1-006 | 0.023 | 0.007 | >5.0 |
| 1-007 | 0.017 | 0.011 | >5.0 |
| 1-008 | 0.016 | 0.046 | >5.0 |
| 1-009 | 0.012 | 0.008 | >5.0 |
| 1-010 | 0.007 | 0.016 | >5.0 |
| 1-011 | 0.014 | 0.015 | >5.0 |
| 1-012 | 0.023 | 0.019 | >5.0 |
| 1-013 | 0.017 | 0.017 | >5.0 |
| 1-014 | 0.010 | 0.009 | >5.0 |
| 1-015 | 0.022 | 0.021 | >5.0 |
| 1-016 | 0.010 | 0.045 | >5.0 |
| 1-017 | 0.017 | 0.030 | >5.0 |
| 1-018 | 0.017 | 0.028 | >5.0 |
| 1-019 | 0.013 | 0.019 | >5.0 |
| 1-020 | 0.012 | 0.015 | >5.0 |
| 1-021 | 0.021 | 0.040 | >5.0 |
| 1-022 | 0.013 | 0.058 | >5.0 |
| 1-023 | 0.021 | 0.070 | >5.0 |
| 1-024 | 0.282 | 0.426 | >5.0 |
| 1-025 | 0.137 | 0.338 | >5.0 |
| 1-026 | 0.054 | 0.111 | >5.0 |
| 1-027 | 0.047 | 0.091 | >5.0 |
| 1-028 | 0.037 | 0.069 | >5.0 |
| 1-029 | 0.041 | 0.094 | >5.0 |
| 1-030 | 0.93 | NT | NT |
| 1-031 | 1.05 | NT | NT |
| 1-032 | 0.020 | 0.104 | 3.36 |
| 1-033 | 0.012 | 0.015 | >5.0 |
| 1-034 | 0.059 | 0.110 | >5.0 |
| 1-035 | 0.075 | 0.145 | >5.0 |
| 1-036 | 0.108 | 0.140 | >5.0 |
| 1-037 | 0.044 | 0.133 | >5.0 |
| 1-038 | 0.028 | 0.051 | >5.0 |
| 1-039 | 0.045 | 0.215 | >5.0 |
| 1-040 | 0.021 | 0.067 | >5.0 |
| 1-041 | 0.088 | 0.309 | >5.0 |
| 1-042 | 0.014 | 0.018 | >5.0 |
| 1-043 | 0.016 | 0.019 | >5.0 |
| 1-044 | 0.096 | 0.152 | >5.0 |
| 1-045 | 0.013 | 0.025 | >5.0 |
| 1-046 | 0.014 | 0.011 | >5.0 |
| 1-047 | 0.016 | 0.028 | >5.0 |
| 1-048 | 0.043 | 0.293 | >5.0 |
| 1-049 | 0.026 | 0.040 | >5.0 |
| 1-050 | 0.013 | 0.037 | >5.0 |
| 1-051 | 0.075 | 0.115 | >5.0 |
| 1-052 | 0.023 | 0.014 | >5.0 |
| 1-053 | 0.186 | NT | NT |
| 1-054 | 0.014 | 0.007 | >5.0 |
| 1-055 | 0.021 | 0.036 | >5.0 |
| 1-056 | 0.020 | 0.029 | >5.0 |
| 1-057 | 0.015 | 0.011 | >5.0 |
| 1-058 | 0.032 | 0.027 | >5.0 |
| 1-059 | 0.719 | NT | NT |
| 1-060 | 0.018 | 0.005 | >5.0 |
| 1-061 | 0.489 | NT | NT |
| 1-062 | 0.518 | NT | NT |
| 1-063 | 0.007 | 0.009 | >5.0 |
| 1-064 | 0.007 | 0.015 | >5.0 |
| 1-065 | 0.018 | 0.055 | >5.0 |
| 1-066 | 0.031 | 0.101 | >5.0 |
| 1-067 | 0.012 | 0.048 | >5.0 |
| 1-068 | 0.026 | 0.065 | >5.0 |
| 1-069 | 0.029 | 0.034 | >5.0 |
| 1-070 | 0.037 | 0.098 | >5.0 |
| 1-072 | 0.015 | 0.031 | >5.0 |
| 1-073 | 0.342 | NT | NT |
| 1-074 | 0.344 | NT | NT |
| 1-075 | 0.024 | 0.034 | >5.0 |
| 1-076 | 0.012 | 0.011 | >5.0 |
| 1-077 | 0.001 | 0.041 | >5.0 |
| 1-078 | 0.015 | 0.077 | >5.0 |
| 1-079 | 0.016 | 0.073 | >5.0 |
| 1-080 | 0.031 | 0.044 | >5.0 |
| 1-081 | 0.021 | 0.040 | >5.0 |
| 1-082 | 0.354 | NT | NT |
| 1-083 | 0.015 | 0.020 | >5.0 |
| 1-084 | 0.107 | NT | NT |
| 1-085 | 0.012 | 0.052 | >5.0 |
| 1-086 | 0.006 | 0.012 | >5.0 |
| 1-087 | 0.018 | 0.023 | >5.0 |
| 1-088 | 0.022 | 0.041 | >5.0 |
| 1-089 | 0.039 | 0.036 | >5.0 |
| 1-090 | 0.019 | 0.013 | >5.0 |
| 1-091 | 0.022 | 0.011 | >5.0 |
| 1-092 | 0.008 | 0.018 | >5.0 |
| 1-093 | 0.034 | 0.031 | >5.0 |
| 1-094 | 0.017 | 0.048 | >5.0 |
| 1-095 | 0.015 | 0.019 | >5.0 |
| 1-096 | 0.017 | 0.066 | >5.0 |
| 1-097 | 0.099 | 0.092 | >5.0 |
| 1-098 | 0.079 | 0.181 | >5.0 |
| 1-099 | 0.092 | 0.237 | >5.0 |
| 1-100 | 0.103 | 0.092 | >5.0 |
| 1-101 | 1.310 | NT | NT |
| 1-102 | 0.471 | NT | NT |
| 1-103 | 1.00 | 0.522 | >5.0 |
| 1-104 | 0.371 | 0.354 | >5.0 |
| 1-105 | 0.252 | 0.213 | >5 |
| 1-106 | 0.084 | NT | NT |
| 1-107 | 0.008 | 0.029 | >5 |
| 1-108 | 0.004 | 0.012 | >5 |
| 1-109 | 0.047 | NT | NT |

NT = not tested

The results presented in Table 3 have been generated with the in vitro assays described above. These assays may be used to test any other compound described herein to assess and characterize a compound's biological activity. In view of the disclosure provided herein, compounds not specifically tested would be expected to have similar results.

Compounds showing activity in the coupled exchange assay are useful in the methods provided herein (see Section “METHODS OF USE”). See, e.g., Lanman et al., 2020; Hong et al., 2020. The inhibitory effect on tumor growth of the compounds provided herein can be shown, for example, using the following animal model.

Tumor cells are cultured, harvested and implanted subcutaneously into the right flank of female athymic nude mice. When tumors reach about 200 mm³, mice are randomized into treatment groups (n=10/group) and treatment is initiated (on days indicated on graphs). Tumor sizes and body weights are measured 2 to 3 times per week. Tumor volume is measured by digital calipers, calculated as L×W×H and expressed in mm³. Statistical significance of observed differences between growth curves can be evaluated by repeated measures analysis of covariance (RMANOVA) of the log transformed tumor volume data with Dunnett adjusted multiple comparisons comparing the control group to the treatment group. For combination studies, RMANOVA can be run with the combination group compared one to one with each single agent treatment group.

The foregoing description is provided for clearness of understanding only. No unnecessary limitations should be understood therefrom, as modifications within the scope of the invention may be apparent to those having ordinary skill in the art.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The invention illustratively disclosed herein suitably may be practiced in the absence of any element or step which is not specifically disclosed herein.

What is claimed is:

1. A compound of Formula (I): 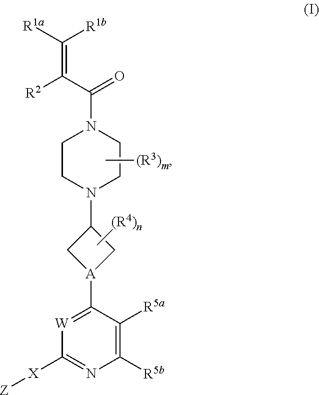
or a pharmaceutically acceptable salt thereof,
wherein:
m is 0, 1, 2, 3, or 4;
n is 1 or 2;
o is 0, 1, 2, 3, or 4;
A is N, CH, C-halo, C—CN, C—C₁₋₃alkyl, C—C₁₋₃haloalkyl, C—C₀₋₇alkyleneOH, or C—C₀₋₃alkylene-C₁₋₄alkoxy;
W is CH, C-halo, C—CN, C—C₁₋₃alkyl, C—C₁₋₃haloalkyl, C—C₀₋₃alkyleneOH, or C—C₀₋₃alkylene-C₁₋₁alkoxy;
X is 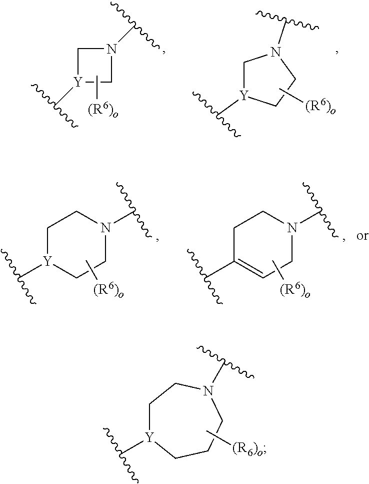
Y is N, C—H, C-halo, C—CN, C—C₁₋₃alkyl, C—C₁₋₃haloalkyl, C—C₀₋₃alkyleneOH, or C—C₀₋₃alkylene-C₁₋₄alkoxy;
Z is phenyl, heteroaryl comprising 5 or 6 total ring atoms and 1-3 heteroatoms selected from N, O, and S, or a bicyclic ring comprising a heteroaryl ring having 5 or 6 total ring atoms and 1-3 heteroatoms selected from N, O, and S fused to a cycloalkyl ring having 5 or 6 total ring atoms or a heterocycloalkyl ring having 5 or 6 total ring atoms and 1 or 2 heteroatoms selected from N, O, and S; wherein each of the phenyl, heteroaryl, and bicyclic rings is optionally substituted with 1-4 substituents;
each of R^{1a}, R^{1b}, and R² independently is H, D, halo, C₁₋₄alkyl, C₁₋₄haloalkyl, C₁₋₂alkylene-OH, C₀₋₂alkylene-C₁₋₄alkoxy, C₀₋₂alkylene-C₁₋₄haloalkoxy, C₀₋₂alkylene-CN, C₀₋₂alkylene-N(R^{N1})₂, C₁₋₂alkylene-heterocycloalkyl having 3-6 total ring atoms and 1 or 2 heteroatoms selected from N, O, and S, or R^{1b} and R², together with the carbon atoms to which they are attached, from a 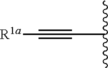
group;
each R³ independently is C₁₋₃alkyl, C₁₋₃haloalkyl, C₀₋₃alkyleneCN, C₀₋₃alkyleneOH, C₀₋₃alkylene-C₁₋₃alkoxy, oxo, spiro-cycloalkyl having 3-7 total ring atoms, spiro-heterocycloalkyl having 3-7 total ring atoms and 1 or 2 heteroatoms selected from N, O and S, or two adjacent R³, together with the atoms to which they are attached, form a fused cycloalkyl ring having 3-7 total ring atoms;
one R⁴ and R^{5a}, together with the atoms to which they are attached, form an optionally substituted ring having 6-10 total ring atoms and 0, 1, or 2 heteroatoms selected from N, O, and S, wherein the ring is saturated or unsaturated;
when n is 2, the other R⁴ is C₁₋₃alkyl, C₁₋₃haloalkyl, C₀₋₃alkyleneCN, C₁₋₂alkyleneOH, C₁₋₂alkylene-C₁₋₃alkoxy, oxo, spiro-cycloalkyl having 3-7 total ring atoms, or spiro-heterocycloalkyl having 4-7 total ring atoms and 1 or 2 heteroatoms selected from N, O and S;
R^{5b} is C₁₋₃haloalkyl, C₁₋₄alkyl, C₂₋₃alkenyl, C₂₋₃alkynyl, halo, C₁₋₃alkoxy, C₁₋₃thioalkoxy, cycloalkyl having 3-7 total ring atoms, cycloalkenyl having 5-7 total ring atoms, heterocycloalkyl having 3-7 total ring atoms and 1-3 heteroatoms selected from N, O, and S, or heterocycloalkenyl having 5-7 total ring atoms and 1-3 heteroatoms selected from N, O, and S, wherein each of foregoing is independently optionally substituted with 1-3 substituents;
each R⁶ independently is halo, CN, oxo, C₁₋₃alkyl, C₁₋₄haloalkyl, C₀₋₃alkyleneOH, C₀₋₃alkylene-C₁₋₃alkoxy, deuterated C₀₋₆alkylene-C₁₋₃alkoxy, C₁₋₄alkylene-N(R^{N1})₂, spiro-cycloalkyl having 3-7 total ring atoms, spiro-heterocycloalkyl having 4-7 total ring atoms and 1 or 2 heteroatoms selected from N, O and S; or two adjacent R⁶, together with the atoms to which they are attached, form a fused cycloalkyl ring having 3-7 total ring atoms; or Y and an adjacent R⁶, together with the atoms to which they are attached, form a fused cycloalkyl ring having 3-7 total ring atoms; wherein the fused cycloalkyl ring of any of the foregoing is optionally substituted with 1 or 2 substituents; or
two non-adjacent R⁶join together to form a C₁₋₃alkylene bridge or a C₁₋₃ether bridge; and
each R^{N1} independently is H or C₁₋₄alkyl.

2. The compound or salt of claim 1, wherein 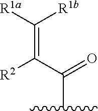
is

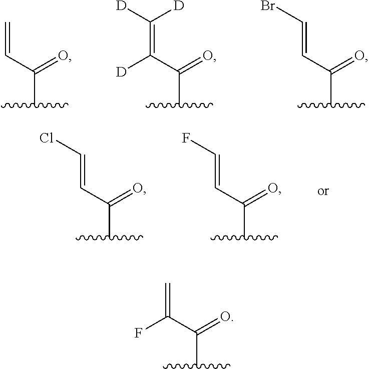

3. The compound or salt of claim 1 or 2, wherein 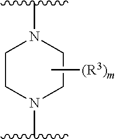
is

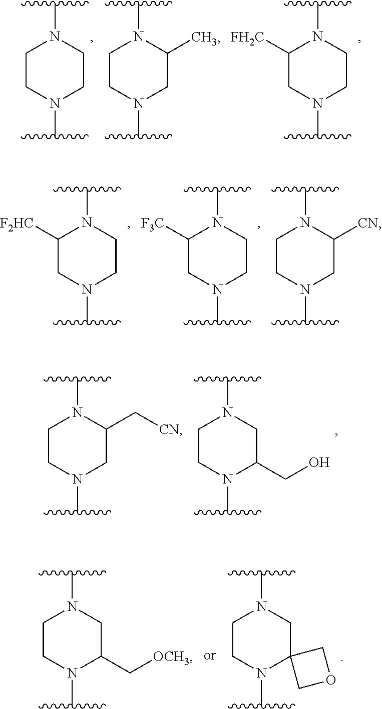

4. The compound or salt of any one of claims 1-3, wherein A is N.

5. The compound or salt of any one of claims 1-4, wherein n is 1.

6. The compound or salt of any one of claims 1-4, wherein n is 2.

7. The compound or salt of claim 6, wherein the other R⁴ is CH₃, CH₂CH₃, CH₂CH₂CH₃, CH(CH₃)₂, CF₃, CHF₂, or CH₂F.

8. The compound or salt of any one of claims 1-7, wherein W is CH.

9. The compound or salt of any one of claims 1-8, wherein one R⁴ and R^{5a}, together with the atoms to which they are attached, form an optionally substituted ring having 6-10 total ring atoms and 0, 1, or 2 heteroatoms selected from N, O, and S, wherein the ring is saturated.

10. The compound or salt of any one of claims 1-8, wherein one R⁴ and R^{5a}, together with the atoms to which they are attached, form an optionally substituted ring having 6-10 total ring atoms and 0, 1, or 2 heteroatoms selected from N, O, and S, wherein the ring is unsaturated.

11. The compound or salt of any one of claims 1-10, wherein the optionally substituted ring formed by one R⁴ and R^{5a}, together with the atoms to which they are attached, has 6 or 7 total ring atoms.

12. The compound or salt of any one of claims 1-11, wherein the optionally substituted ring formed by one R⁴ and R^{5a}, together with the atoms to which they are attached, has 0 heteroatoms.

13. The compound or salt of any one of claims 1-11, wherein the optionally substituted ring formed by one R⁴ and R^{5a}, together with the atoms to which they are attached, has 1 or 2 heteroatoms selected from N, O, and S.

14. The compound or salt of claim 13, wherein the 1 or 2 heteroatoms are each O.

15. The compound or salt of claim 13, wherein the 1 or 2 heteroatoms are each N.

16. The compound or salt of any one of claims 1-15, wherein the ring formed by one R⁴ and R^{5a}, together with the atoms to which they are attached, is unsubstituted.

17. The compound or salt of any one of claims 1-15, wherein the ring formed by one R⁴ and R^{5a}, together with the atoms to which they are attached, is substituted with 1 or 2 substituents selected from the group consisting of C₁₋₃alkyl, C₁₋₃haloalkyl, oxo, halo, CN, C₀₋₆alkyleneOH, C₀₋₃alkylene-C₁₋₃alkoxy, cycloalkyl having 3-7 total ring atoms, cycloalkenyl having 5-7 total ring atoms, heterocycloalkyl having 4-7 total ring atoms and 1-3 heteroatoms selected from N, O, and S, heterocycloalkenyl having 5-7 total ring atoms and 1-3 heteroatoms selected from N, O, and S, and phenyl.

18. The compound or salt of claim 1, wherein 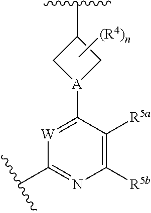
is

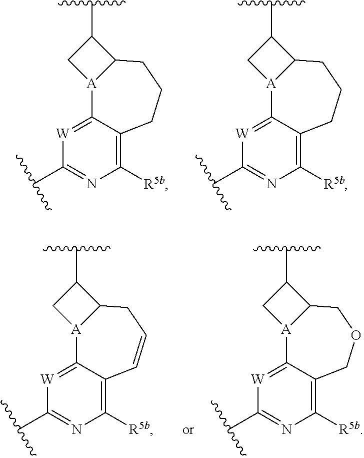

19. The compound or salt of any one of claims 1-18, wherein R^{5b} is CF₃, CF₂H, CFH₂, of CF₂CH₃.

20. The compound or salt of any one of claims 1-19, wherein X is

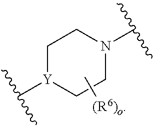

21. The compound or salt of any one of claim 1-20, wherein Y is C—H.

22. The compound or salt of any one of claims 1-21, wherein o is 0.

23. The compound or salt of any one of claims 1-21, wherein o is 1.

24. The compound or salt of claim 23, wherein R⁶ is CH₃, CH₂F, CHF₂, or CF₃.

25. The compound or salt of claim 20, wherein 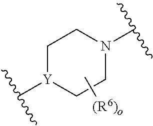
is

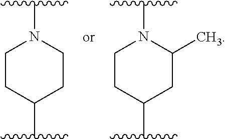 [SMILES: *C1CCN(*)CC1 ; *C1CCN(*)C(C)C1]

26. The compound or salt of any one of claims 1-25, wherein Z is heteroaryl comprising 5 or 6 total ring atoms and 1-3 heteroatoms selected from N, O, and S, wherein the heteroaryl is optionally substituted with 1-4 substituents.

27. The compound or salt of claim 26, wherein the heteroaryl is pyrrolyl, furanyl, thiophenyl, pyrazolyl, imidazolyl, thiazolyl, isothiazolyl, oxazolyl, isoxazolyl, triazolyl, thiadiazolyl, oxadiazolyl, pyridyl, pyridazinyl, pyrimidinyl, pyrazinyl, or triazinyl.

28. The compound or salt of claim 27, wherein the heteroaryl is pyrazolyl or pyridyl.

29. The compound or salt of any one of claims 26-28, wherein the heteroaryl is substituted with 1-4 substituents, each of which independently is selected from the group consisting of halo, CN, C₁₋₆alkyl, C₁₋₆haloalkyl, C₂₋₆alkenyl, C₂₋₆haloalkenyl, C₀₋₆alkylene-OH, C₀₋₆alkylene-C₁₋₃alkoxy, C₀₋₆alkylene-N(R^{N1}), wherein each R^{N1} independently is H or C₁₋₃alkyl, C₀₋₂alkylene-cycloalkyl having 3-6 total ring atoms, C₀₋₂alkylene-heterocycloalkyl having 3-6 total ring atoms and 1-3 heteroatoms selected from N, O, and S, and C₀₋₂alkylene-phenyl; wherein each of the alkyl, alkenyl, C₀₋₆alkylene-C₁₋₃alkoxy, cycloalkyl, heterocycloalkyl, and phenyl substituents independently is optionally substituted with 1-3 substituents independently selected from deuterium, halo, OH, CH₃, OCH₃, and OCD₃.

30. The compound or salt of claim 29, wherein each of the 1-4 substituents independently is selected from the group consisting of Cl, F, CN, CH₃, CD₃, CH₂CH₃, CH(CH₃)₂, CF₃, CHF₂, CH₂F, CH₂CHF₂, CH₂CH₂F, CH(CH₂F)₂, CH(CH₃)CH₂F, CH(CH₃)CHF₂, C(═CH₂)CH₂F, OH, CH₂OH, CH₂CH₂OH, CH(CH₃)CH₂OH, C(CH₃)₂OH, C(CH₃)₂CH₂OH, CH₂C(CH₃)₂OH, OCH₃, OCD₃, CH₂OCH₃, CH₂OCD₃, CH₂CH₂OCH₃, CHFCH₂OCH₃, CF₂CH₂OCH₃, CH₂CH₂OCD₃, CH₂CH₂OCH₂CH₃,CH₂CH₂CH₂OCH₃, CH₂CH₂CH₂OCD₃, CH(CH₃)CH₂OCH₃, CH(CH₃)CH₂OCD₃,C(CH₃)₂CH₂OCH₃, C(CH₃)₂CH₂OCD₃, CH₂CH(CH₃)OCH₃, CH₂CH(CH₃)OCD₃, CH₂C(CH₃)₂OCH₃, CH₂C(CH₃)₂OCD₃, NH₃, CH₂NH₂, CH₂NHCH₃, CH₂N(CH₃)₂, CH₂CH₂NH₂, CH₂CH₂NHCH₃, CH₂CH₂N(CH₃)₂,

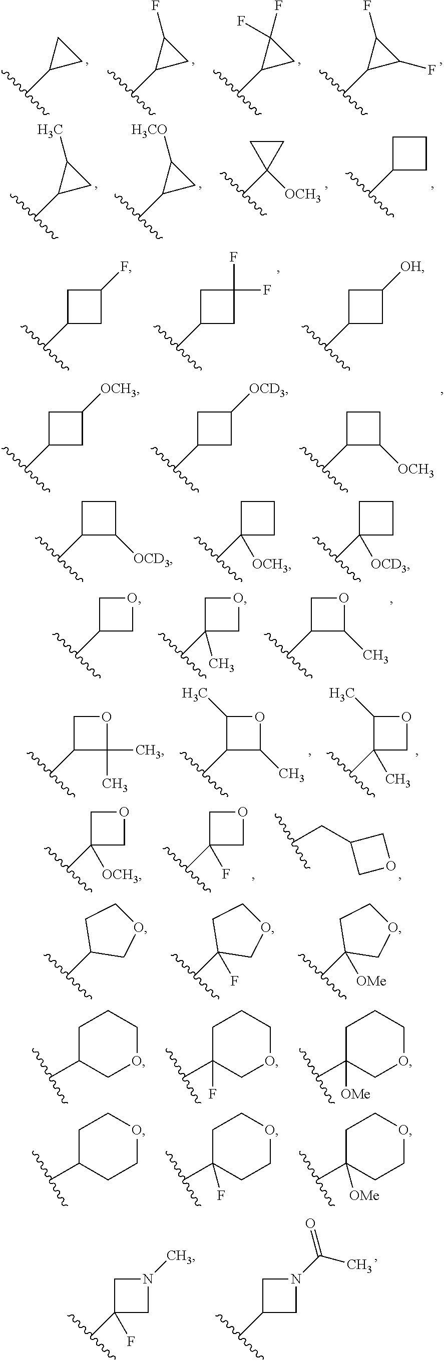

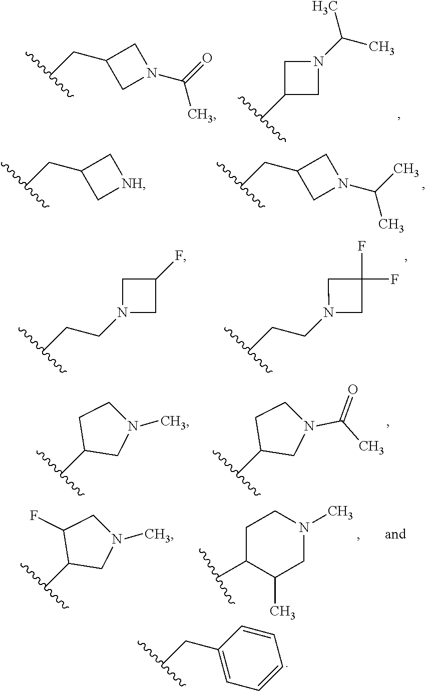

31. The compound or salt of claim 30, wherein each of the 1-4 substituents independently is CH₃, CH₂CH₂OCH₃, CH₂CH₂OCD₃,

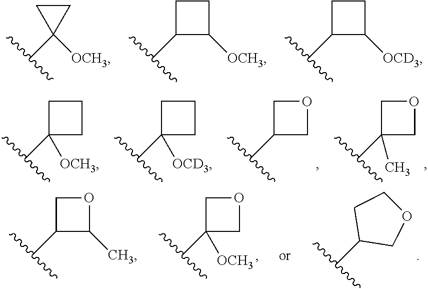

32. The compound or salt of any one of claims 26-30, wherein Z is

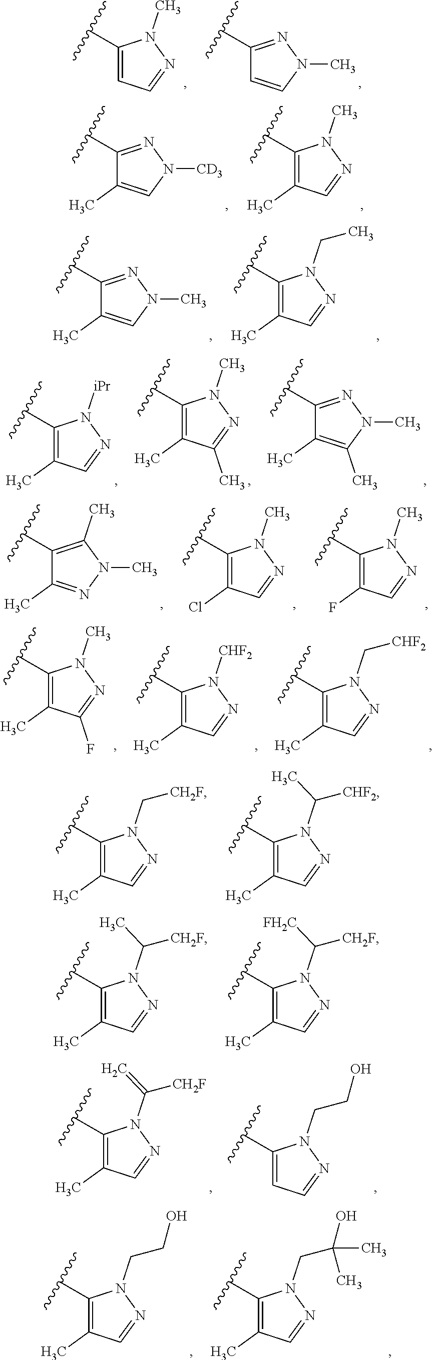

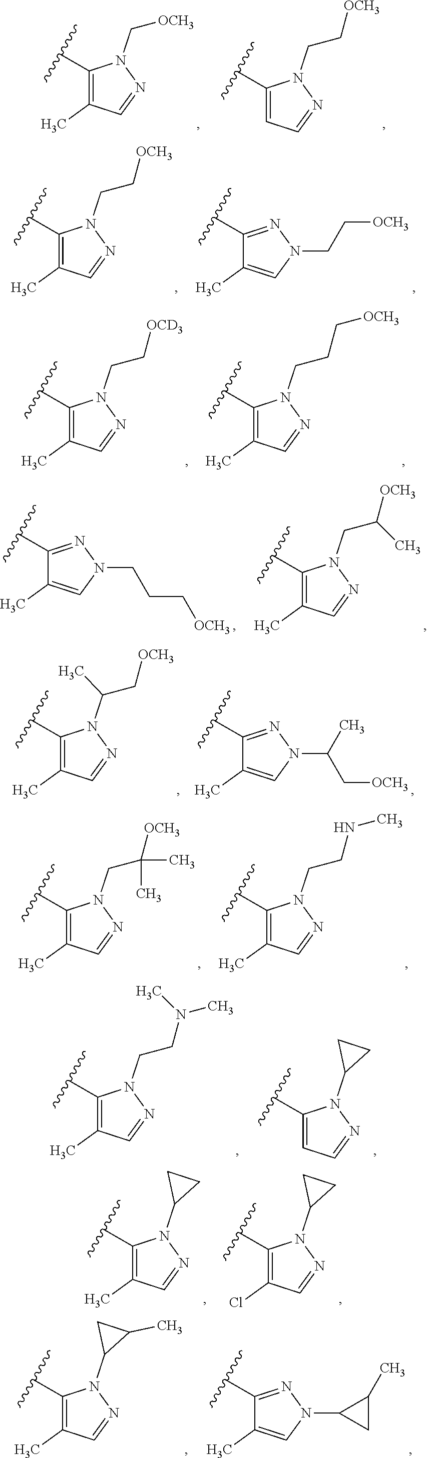

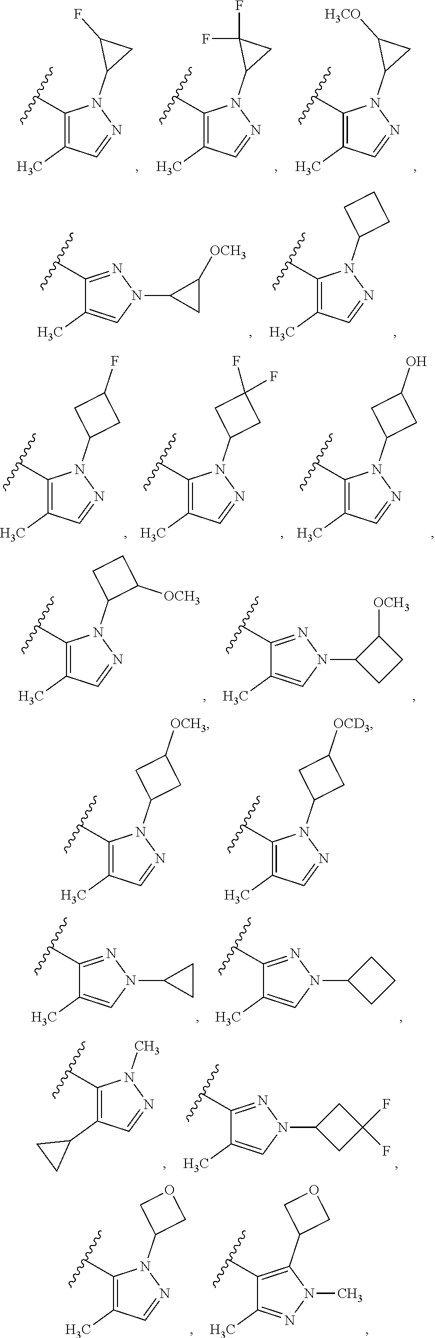

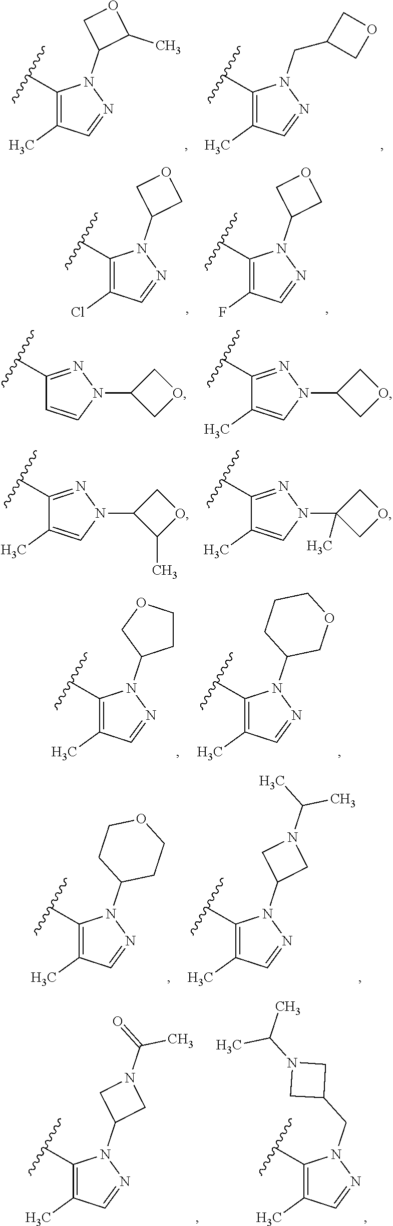

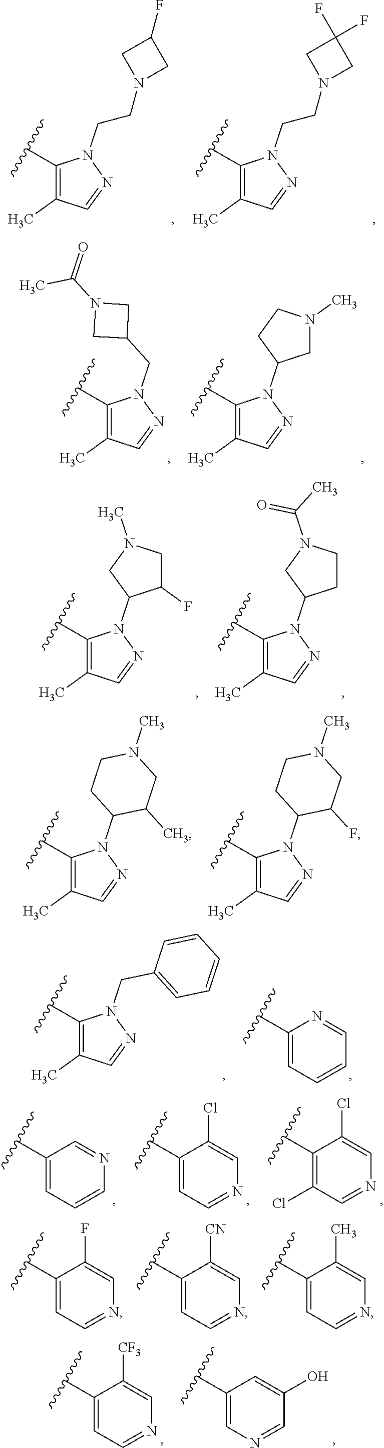

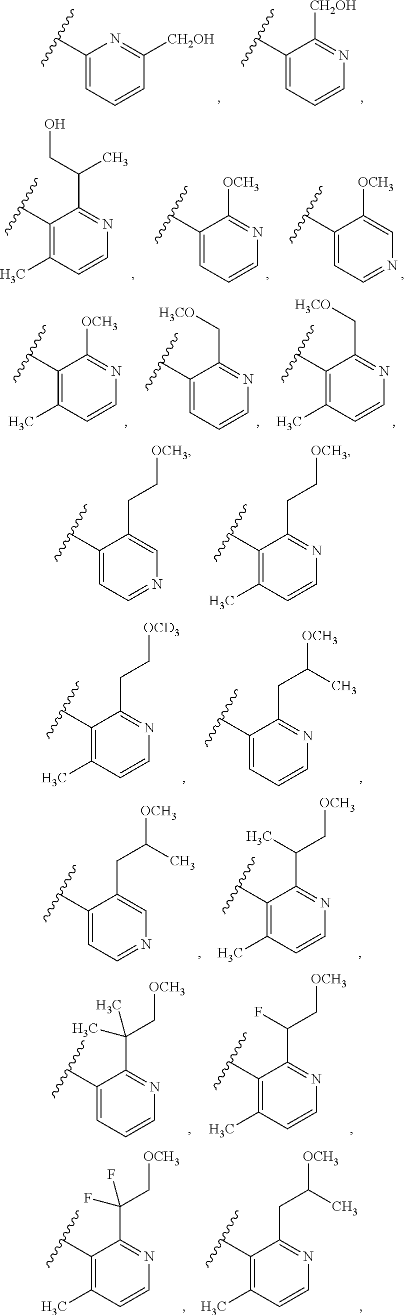

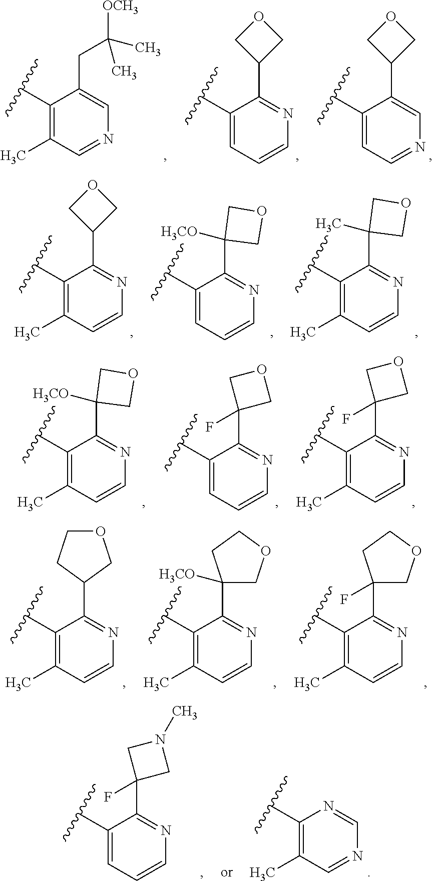

33. The compound or salt of claim 32, wherein Z is

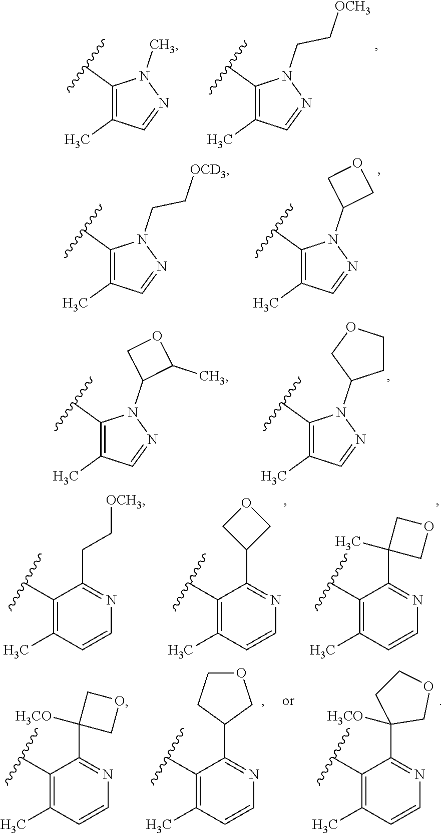

34. The compound or salt of claim 1, wherein: 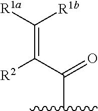
is 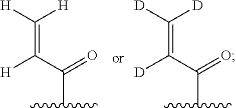 [SMILES: *C(=O)C=C ; "or" ; *C(=O)C([2H])=C([2H])[2H]] 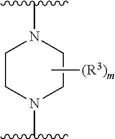
is 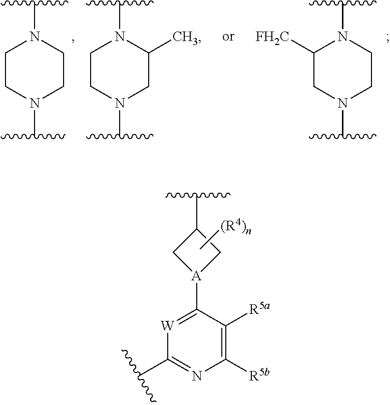
is 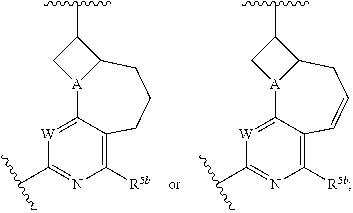
X is 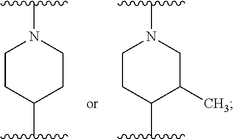 [SMILES: *C1CCN(*)CC1 ; *C1CCN(*)CC1C]
and
Z is pyrazolyl or pyridyl, each of which is optionally substituted with 1-4 substituents.

35. The compound or salt of claim 34, wherein each of the 1-4 substituents of Z independently is CH₃, CH₂CH₂OCH₃, CH₂CH₂OCD₃,

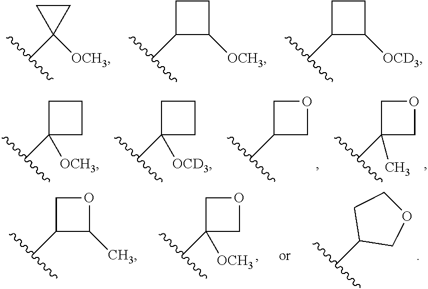

36. The compound or salt of claim 34 or 35, wherein Z is substituted with 2 substituents.

37. The compound or salt of claim 36, wherein one substituent is CH₃.

38. The compound or salt of claim 37, wherein the other substituent is CH₃, CH₂CH₂OCH₃,

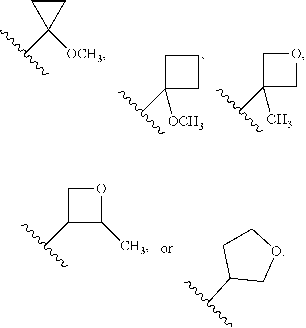

39. The compound or salt of any one of claims 34-38, wherein Z is

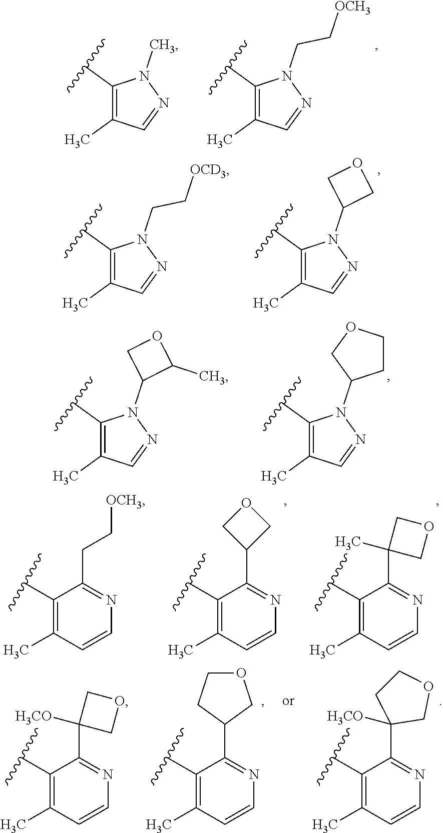

40. The compound or salt of claim 39, wherein Z is

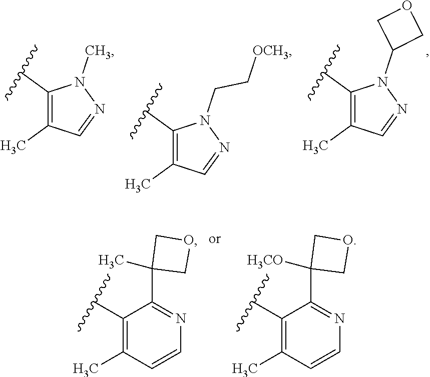

41. The compound or salt of claim 1 having a structure: 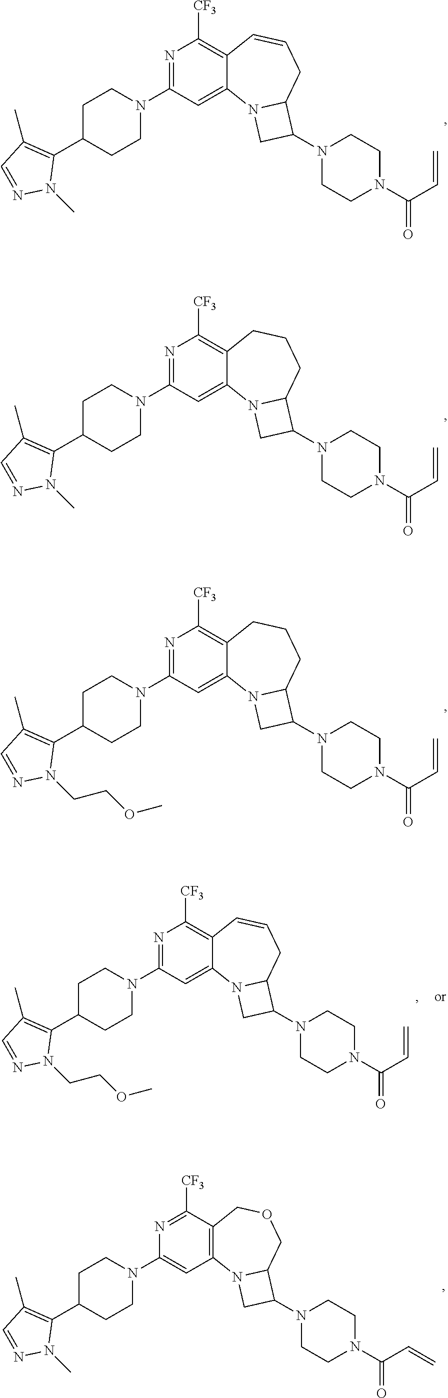 [SMILES: C=CC(=O)N1CCN(C2CN3c4cc(N5CCC(c6c(C)cnn6C)CC5)nc(C(F)(F)F)c4C=CCC23)CC1 ; C=CC(=O)N1CCN(C2CN3c4cc(N5CCC(c6c(C)cnn6C)CC5)nc(C(F)(F)F)c4CCCC23)CC1 ; C=CC(=O)N1CCN(C2CN3c4cc(N5CCC(c6c(C)cnn6CCOC)CC5)nc(C(F)(F)F)c4CCCC23)CC1 ; C=CC(=O)N1CCN(C2CN3c4cc(N5CCC(c6c(C)cnn6CCOC)CC5)nc(C(F)(F)F)c4C=CCC23)CC1 ; C=CC(=O)N1CCN(C2CN3c4cc(N5CCC(c6c(C)cnn6C)CC5)nc(C(F)(F)F)c4COCC23)CC1]
or a pharmaceutically acceptable salt thereof.

42. The compound of claim 41 having a structure: 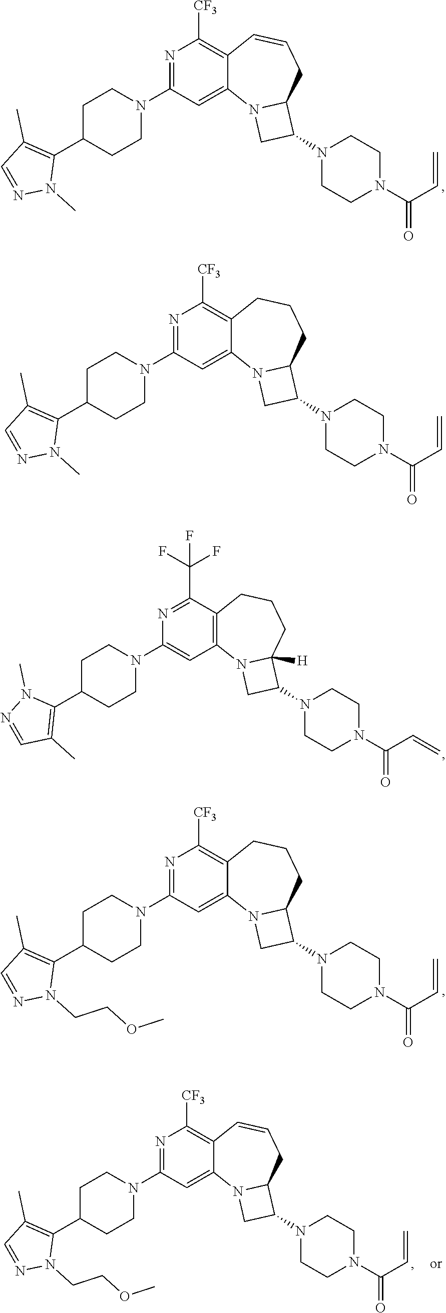 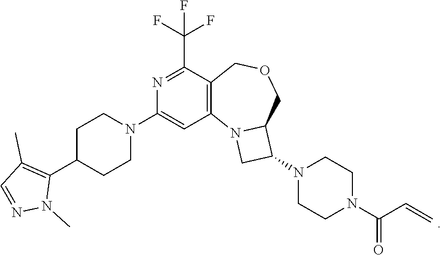 [SMILES: C=CC(=O)N1CCN([C@@H]2CN3c4cc(N5CCC(c6c(C)cnn6C)CC5)nc(C(F)(F)F)c4COC[C@@H]23)CC1]
or a pharmaceutically acceptable salt thereof.

43. A compound of Formula (II): 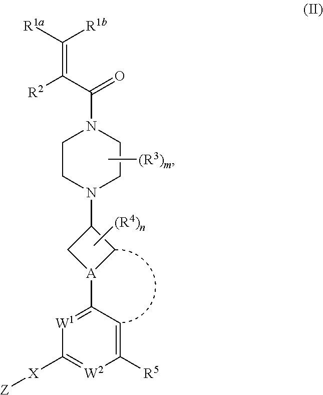
or a pharmaceutically acceptable salt thereof,
wherein:
m is 0, 1, 2, 3, or 4;
n is 0, 1, or 2;
A is N, CH, C-halo, C—CN, C—C₁₋₃alkyl, C₁₋₃haloalkyl, C—C₀₋₃alkyleneOH, or C—C₀₋₃alkylene-C₁₋₄alkoxy;
each of W¹ and W² independently is N, CH, C-halo, C—CN, C—C₁₋₃alkyl, C—C₂₋₃alkenyl, C—C₂₋₃alkynyl, C—C₁₋₃haloalkyl, C—C₀₋₂alkyleneOH, or C—C₀₋₃alkylene-C₁₋₄alkoxy, wherein each of the alkenyl and alkynyl is unsubstituted or substituted with 1-3 substituents and each substituent independently is halo, C₁₋₃haloalkyl, C₀₋₆alkyleneOH, or C₀₋₂alkyleneC₁₋₄alkoxy;
X is heterocycloalkyl or heterocycloalkenyl, each having 4-7 total ring atoms and 1-3 heteroatoms selected from N, O, and S, wherein each of the heterocycloalkyl and heterocycloalkenyl is unsubstituted or substituted with 1-3 substituents, and each substituent independently is halo, C₁₋₃alkyl, C₁₋₃haloalkyl, C₀₋₂alkyleneOH, C₀₋₂alkyleneC₁₋₃alkoxy, or C₀₋₂alkyleneCN;
Z is phenyl, heteroaryl comprising 5 or 6 total ring atoms and 1-3 heteroatoms selected from N, O, and S, or a bicycle ring comprising a heteroaryl having 5 or 6 total ring atoms and 1-3 heteroatoms selected from N, O, and S fused C₅₋₆cycloalkyl or a heterocycloalkyl ring having 5 or 6 total ring atoms and 1 or 2 heteroatoms selected from N, O, and S;
wherein each of the phenyl, heteroaryl, and bicyclic ring is unsubstituted or substituted with 1-4 substituents, and each substituent independently is halo, CN, C₁₋₃alkyl, C₁₋₆haloalkyl, C₃₋₆alkenyl, C₂₋₆haloalkenyl, C₀₋₆alkylene-OH, C₀₋₃alkylene-C₁₋₃alkoxy, C₀₋₆alkylene-N(R^{N1})₂, C₀₋₂alkylene-C₃-cycloalkyl, C₀₋₂alkylene-heterocycloalkyl having 3-6 total ring atoms and 1-3 heteroatoms selected from N, O, and S, or C₀₋₂alkylene-phenyl;
wherein each of the C₁₋₃alkyl, C₂₋₆alkenyl, C₀₋₆alkylene-C₁₋₃alkoxy, cycloalkyl, heterocycloalkyl, and phenyl substituents independently is unsubstituted or substituted with 1-3 further substituents, and each further substituent independently is D, halo, C₁₋₃alkyl, C₁₋₃haloalkyl, C₁₋₂alkyleneOH, C₁₋₂alkylene-C₁₋₃alkoxy, C₁₋₃deuterated alkoxy, N(R^{N1})₂, (C═O)C₁₋₃alkyl, C₃₋₅cycloalkyl, or heterocycloalkyl having 3-5 total ring atoms and 1 or 2 heteroatoms selected from N, O, and S; or two geminal further substituents, together with the atom to which they are attached, form spiro-C₃₋₅cycloalkyl or spiro-heterocycloalkyl having 3-5 total ring atoms and 1 or 2 heteroatoms selected from N, O, and S; or two vicinal further substituents, together with the atoms to which they are attached, form fused-C₃₋₅cycloalkyl or fused-heterocycloalkyl having 3-5 total ring atoms and 1 or 2 heteroatoms selected from N, O, and S;
wherein each of the foregoing cycloalkyl and heterocycloalkyl further substituents independently is unsubstituted or substituted with 1 or 2 substituents, and each substituent independently is halo or C₁₋₃alkyl; [symbol]
is C₀₋₃alkylene, C₂₋₆alkenylene, heteroalkylene having 2-6 total atoms and 1-3 heteroatoms selected from N, O, and S, or heteroalkenylene having 3-6 total atoms and 1 or 2 heteroatoms selected from N, O, and S, wherein [symbol] is unsubstituted or substituted with 1-4 substituents, and each substituent independently is C₁₋₃alkyl, C₁₋₃haloalkyl, C₂₋₃alkenyl, halo, CN, C₀₋₃alkyleneOH, C₀₋₂alkylene-C₁₋₃alkoxy, C₃₋₅cycloalkyl, C₄₋₅cycloalkenyl, heterocycloalkyl having 4 or 5 total ring atoms and 1-3 heteroatoms selected from N, O, and S, heterocycloalkenyl having 4 or 5 total ring atoms and 1-3 heteroatoms selected from N, O, and S, or phenyl; or two geminal substituents, together with the atom to which they are attached, form oxo, =CH₂, spiro-C₃₋₅cycloalkyl, spiro-C₄₋₅cycloalkenyl, spiro-heterocycloalkyl having 3-5 total ring atoms and 1 or 2 heteroatoms selected from N, O and S, or spiro-heterocycloalkenyl having 4 or 5 total ring atoms and 1 or 2 heteroatoms selected from N, O and S; or two vicinal substituents, together with the atoms to which they are attached, form fused-C₃₋₅cycloalkyl, fused-C₄₋₅cycloalkenyl, fused-heterocycloalkyl having 3-5 total ring atoms and 1 or 2 heteroatoms selected from N, O and S or fused-heterocycloalkenyl having 4 or 5 total ring atoms and 1 or 2 heteroatoms selected from N, O and S;
each of R^{1a}, R^{1b}, and R² independently is H, D, halo, C₁₋₄alkyl, C₁₋₄haloalkyl, C₁₋₂alkylene-OH, C₀₋₂alkylene-C₁₋₃alkoxy, C₀₋₂alkylene-C₁₋₄haloalkoxy, C₀₋₂alkylene-CN, C₀₋₂alkylene-N(R^{N1})₂, C₁₋₂alkylene-heterocycloalkyl having 3-6 total ring atoms and 1 or 2 heteroatoms selected from N, O, and S, or R^{1b} and R², together with the carbon atoms to which they are attached, form 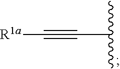
each R³ independently is C₁₋₃alkyl, C₁₋₃haloalkyl, 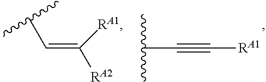
C₀₋₃alkyleneCN, C₀₋₃alkyleneOH, or C₀₋₆alkylene-C₁₋₄alkoxy; or two geminal R³, together with the atom to which they are attached, form oxo, spiro-C₃₋₇cycloalkyl, spiro-C₃₋₇cycloalkenyl, spiro-heterocycloalkyl having 3-7 total ring atoms and 1 or 2 heteroatoms selected from N, O, and S, or spiro-heterocycloalkenyl having 4-7 total ring atoms and 1 or 2 heteroatoms selected from N, O, and S; or two vicinal R³, together with the atoms to which they are attached, form fused-C₃₋₇cycloalkyl, fused-C₄₋₇cycloalkenyl, fused-heterocycloalkyl having 3-7 total ring atoms and 1 or 2 heteroatoms selected from N, O, and S, or fused-heterocycloalkenyl having 4-7 total ring atoms and 1 or 2 heteroatoms selected from N, O, and S; or 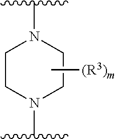
is deuterated;
each R⁴ independently is C₁₋₃alkyl, C₁₋₄haloalkyl, C₀₋₂alkyleneCN, C₁₋₄alkyleneOH, or C₁₋₃alkylene-C₁₋₃alkoxy; or two geminal Rt, together with the atom to which they are attached, form oxo, spiro-C₃, cycloalkyl, or spiro-heterocycloalkyl having 4-7 total ring atoms and 1 or 2 heteroatoms selected from N, O and S;
R³ is halo, C₁₋₃haloalkyl, C₁₋₆alkyl, C₂₋₄alkenyl, C₂₋₄alkynyl, C₁₋₃alkoxy, C₁₋₃thioalkyl, C₃₋₇cycloalkyl, C₅₋₇cycloalkenyl, heterocycloalkyl having 3-7 total ring atoms and 1-3 heteroatoms selected from N, O, and S, or heterocycloalkenyl having 5-7 total ring atoms and 1-3 heteroatoms selected from N, O, and S, wherein each of the foregoing independently is unsubstituted or substituted with 1-3 substituents, and each substituent independently is C₁₋₃haloalkyl, C₀₋₆alkylene-OH, C₀₋₆alkylene-C₁₋₃alkoxy, C₃₋₇cycloalkyl, C₅₋₇cycloalkenyl, heterocycloalkyl having 3-7 total ring atoms and 1-3 heteroatoms selected from N, O, and S, heterocycloalkenyl having 4-7 total ring atoms and 1-3 heteroatoms selected from N, O, and S, or phenyl;
each of R^{A1} and R^{A2} independently is H, C₁₋₃alkyl, C₁₋₃haloalkyl, or C₃₋₅cycloalkyl; and
each R^{N1} independently is H or C₁₋₄alkyl.

44. The compound or salt of claim 43, wherein at least one of R^{1a}, R^{1b}, and R² is H or D.

45. The compound or salt of claim 43 or 44, wherein each of R^{1a}, R^{1b}, and R² independently is H or D.

46. The compound or salt of claim 43 or 44, wherein two of R^{1a}, R^{1b}, and R² are H and one of R^{1a}, R^{1b}, and R² is halo, C₁₋₄alkyl, C₁₋₃haloalkyl, C₁₋₂alkylene-OH, C₀₋₂alkylene-C₁₋₄alkoxy, C₀₋₂alkylene-C₁₋₄haloalkoxy, C₀₋₂alkylene-CN, C₀₋₂alkylene-N(R^{N1})₂, or C₁₋₂alkylene-heterocycloalkyl having 3-6 total ring atoms and 1 or 2 heteroatoms selected from N, O, and S.

47. The compound or salt of claim 43, wherein 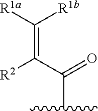
is

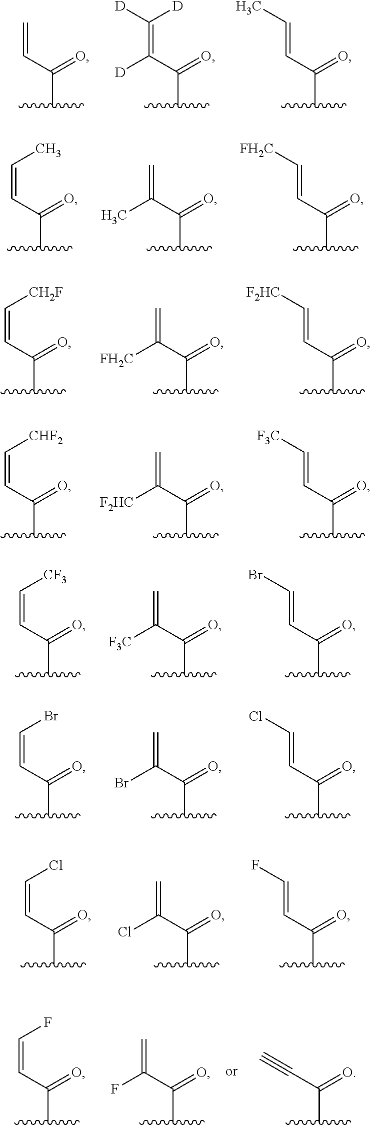

48. The compound or salt of claim 47, wherein 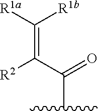
is

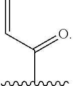 [SMILES: *C(=O)C=C]

49. The compound or salt of any one of claims 1-48, wherein m is 0.

50. The compound or salt of any one of claims 1-48, wherein m is 1.

51. The compound or salt of any one of claims 1-48, wherein m is 2.

52. The compound or salt of any one of claims 43-48, wherein each R³ independently is CH₃, CH₂CH₃, CH₂F, CHF₂, CF₃, CN, CH₂CN, OH, CH₂OH, CH₂CH₂OH, OCH₃, CH₂OCH₃, or CH₂CH₂OCH₃; or two geminal R³, together with the atom to which they are attached, form oxo, spiro-cyclopropyl, spiro-cyclobutyl, spiro-oxetanyl, or spiro-tetrahydrofuranyl; or two vicinal R³, together with the atoms to which they are attached, form fused-cyclopropyl or fused-cyclobutyl.

53. The compound or salt of any one of claims 43-48, wherein m is 0; or m is 1 and R³ is CH₃, CH₂F, CHF₂, CF₃, CN, CH₂CN, CH₂OH, or CH₂OCH₃; or m is 2 and two geminal R³, together with the atom to which they are attached, form spiro-oxetanyl.

54. The compound or salt of claim 53, wherein m is 0; or m is 1 and R³ is CH₃.

55. The compound or salt of any one of claims 42-48, wherein 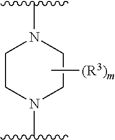
is

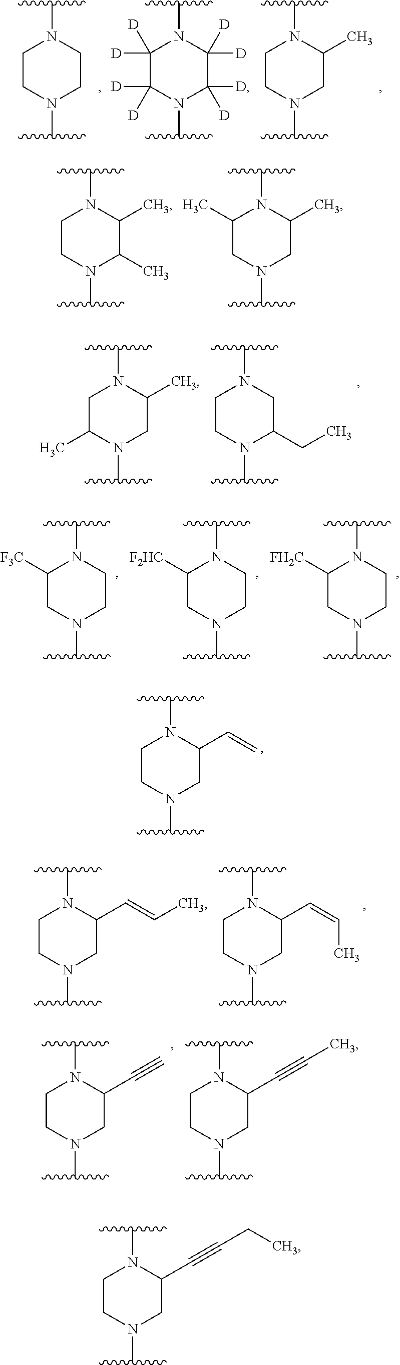

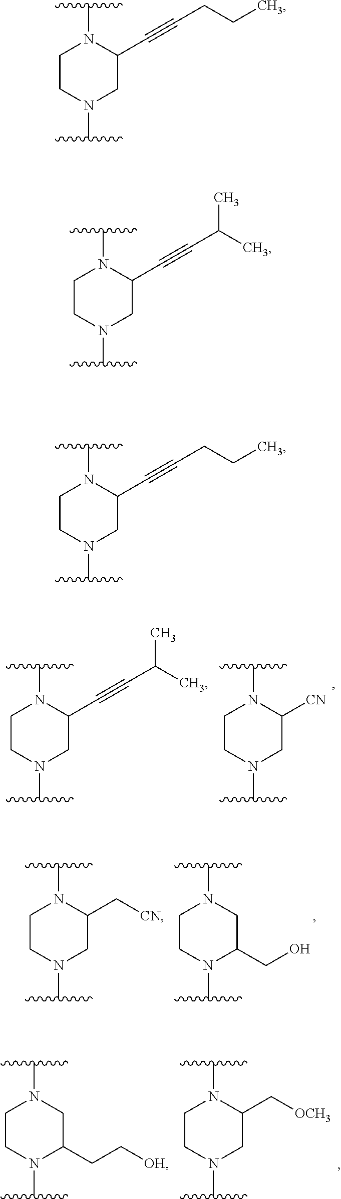

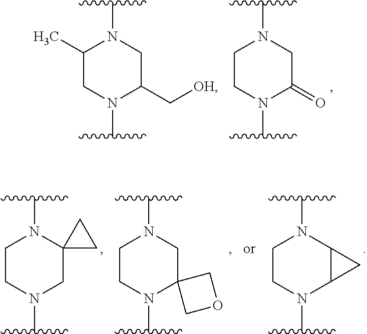

56. The compound or salt of claim 55, wherein 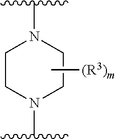
is

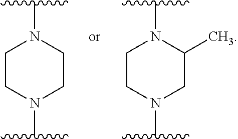 [SMILES: *N1CCN(*)CC1 ; *N1CCN(*)C(C)C1]

57. The compound or salt of any one of claims 43-56, wherein A is N.

58. The compound or salt of any one of claims 43-56, wherein A is CH, C—F, C—Cl, C—CN, C—CH₃, C—CH₂F, C—CHF₂, C—CF₃, C—OH, C—CH₂OH, C—OCH₃, or C—CH₂OCH₃.

59. The compound or salt of any one of claims 46-58, wherein n is 0.

60. The compound or salt of any one of claims 46-58, wherein n is 1.

61. The compound or salt of any one of claims 46-58, wherein n is 2.

62. The compound or salt of any one of claims 46-58, wherein each R⁴ independently is CH₃, CH₂CH₃, CH₂CH₂CH₃, CH(CH₃)₂, CF₃, CHF₂, CH₂F, CN, CH₂CN, CH₂OH, CH₂CH₂OH, CH₂OCH₃, or CH₂CH₂OCH₃; or two geminal R⁴, together with the atom to which they are attached, form oxo, spiro-cyclopropyl, spiro-cyclobutyl, or spiro-oxetanyl.

63. The compound or salt of any one of claims 43-57, wherein 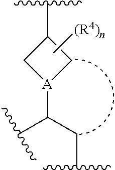
is

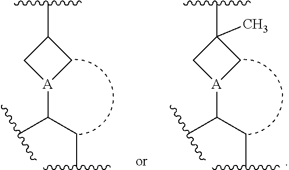

64. The compound or salt of any one of claims 43-63, wherein [symbol] is C₂alkylene, C₃alkylene, C₃alkenylene, or heteroalkylene having 2-4 total atoms and 1 or 2 heteroatoms selected from N, O, and S.

65. The compound or salt of any one of claims 43-64, wherein [symbol] is unsubstituted.

66. The compound or salt of any one of claims 43-64, wherein [symbol] is substituted with CH₃, ═CH₂, oxo, Cl, F, OH, OCH₃, 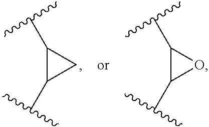 [SMILES: *C1CC1* ; *C1OC1*]
or a combination of the foregoing.

67. The compound or salt of any one of claims 43-64, wherein [symbol] is

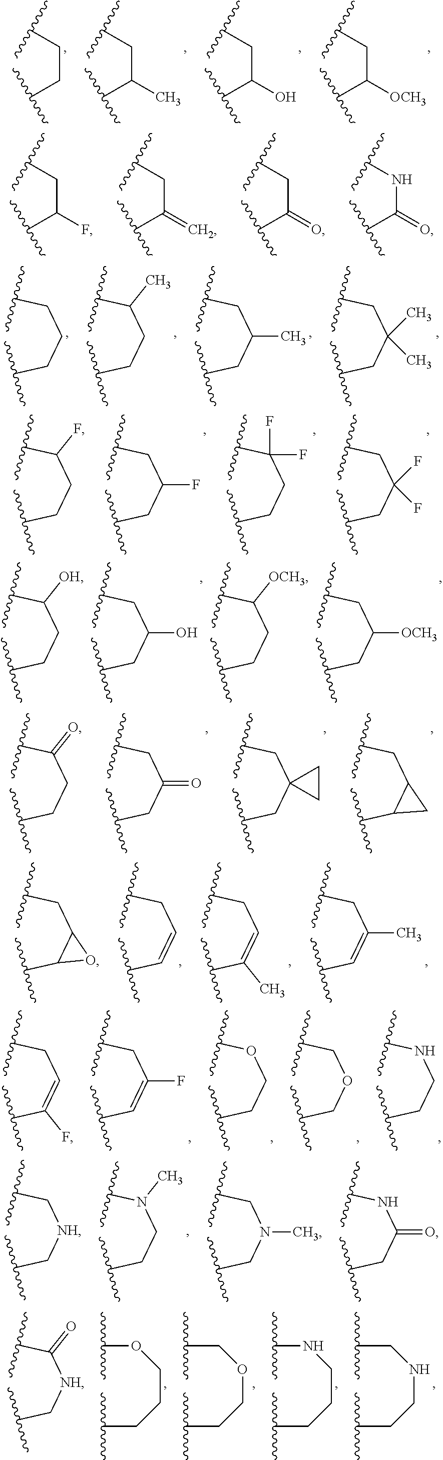

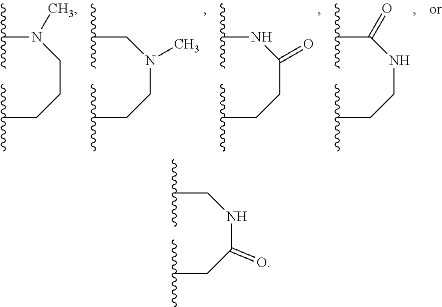

68. The compound or salt of claim 67, wherein [symbol] is

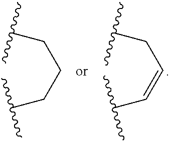 [SMILES: *CCC* ; */C=C\C*]

69. The compound or salt of any one of claims 43-63, wherein [symbol] is 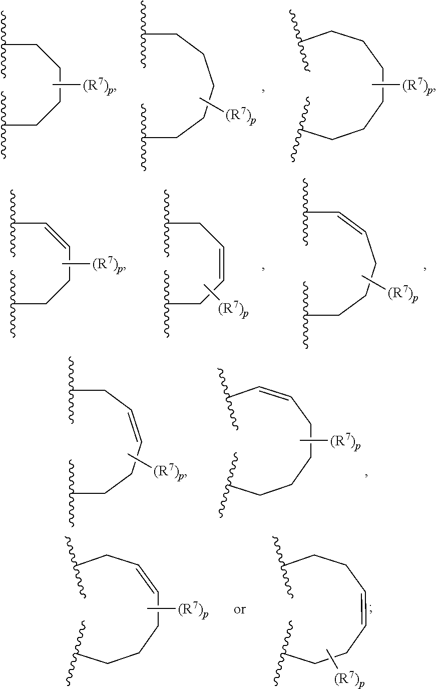
wherein p is 0, 1, 2, or 3, and each R⁷ independently is CH, Cl, F, OH, or OCH₃; or two geminal R⁷, together with the atom to which they are attached form oxo or =CH₂; or two vicinal R⁷, together with the atoms to which they are attached form

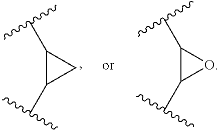 [SMILES: *C1CC1* ; *C1OC1*]

70. The compound or salt of any one of claims 43-69, wherein WI is N.

71. The compound or salt of any one of claims 43-69, wherein W¹ is CH.

72. The compound or salt of any one of claims 43-69, wherein WI is C—F, C—Cl, C—CN, C—CH₃, C—CH₂CH₃, C—CH₂F, C—CHF₂, C—CF₃, C—CH═CH₂, C—C(OH)═CH₂, C—CH═CH(OH), C—CCH, C—OH, C₂CH₂OH, C—OCH₃, or C₂CH₂OCH₃.

73. The compound or salt of any one of claims 43-72, wherein W² is N.

74. The compound or salt of any one of claims 43-72, wherein W² is CH.

75. The compound or salt of any one of claims 43-72, wherein W² is C—F, C—Cl, C—CN, C—CH₃, C—CH₂CH₃, C—CH₂F, C—CHF₂, C—CF₃, C—CH═CH₂, C—C(OH)=CH₂, C—CH═CH(OH), C—CCH, C—OH, C₂CH₂OH, C—OCH₃, or C—CH₂OCH₃.

76. The compound or salt of any one of claim 43-69, wherein WI is CH and W² is N.

77. The compound or salt of any one of claims 43-76, wherein R⁵ is C₁₋₃haloalkyl.

78. The compound or salt of claim 77 wherein R⁵ is CF₃ or CF₂H.

79. The compound or salt of any one of claims 43-76, wherein R⁵ is CH₃, CH₂CH₃, CH₂CH₂CH₃, CH(CH₃)₂, CH═CH₂, CH═CHCH₃, 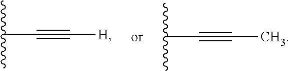
wherein each of the foregoing independently is unsubstituted or substituted with 1-3 substituents, and each substituent independently is C₁₋₃haloalkyl, C₀₋₆alkylene-OH, C₀₋₆alkylene-C₁₋₃alkoxy, C₃₋₇cycloalkyl, C₅₋₇cycloalkenyl, heterocycloalkyl having 3-7 total ring atoms and 1-3 heteroatoms selected from N, O, and S, heterocycloalkenyl having 4-7 total ring atoms and 1-3 heteroatoms selected from N, O, and S, or phenyl.

80. The compound or salt of claim 79, wherein each substituent independently is CH₃, CF₃, CF₂H, CFH₂, OH, OCH₃, OCF₃, CH₂OH, CH₂OCH₃, cyclopropyl, cyclobutyl, or phenyl.

81. The compound or salt of any one of claims 43-76, wherein R⁵ is Br, Cl, F, OCH₃, SCH₃, CH₃, CH₂CH₃, CH₂CH₂CH₃, CH(CH₃)₂,

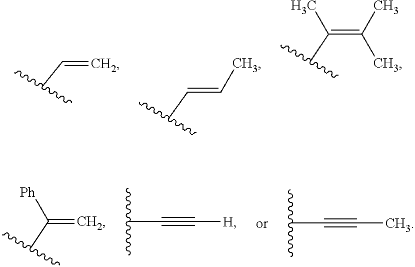

82. The compound or salt of any one of claims 43-69, wherein W¹ is CH, W² is N, and R⁵ is CF₃, CF₂H, or CFH₂.

83. The compound or salt of any one of claims 43-63, wherein 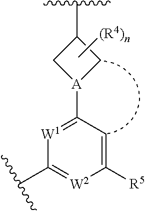
is

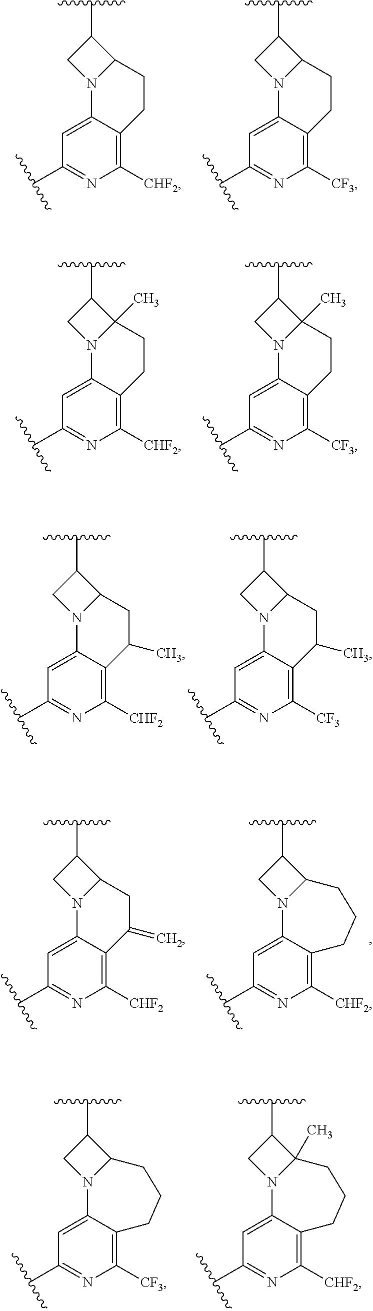

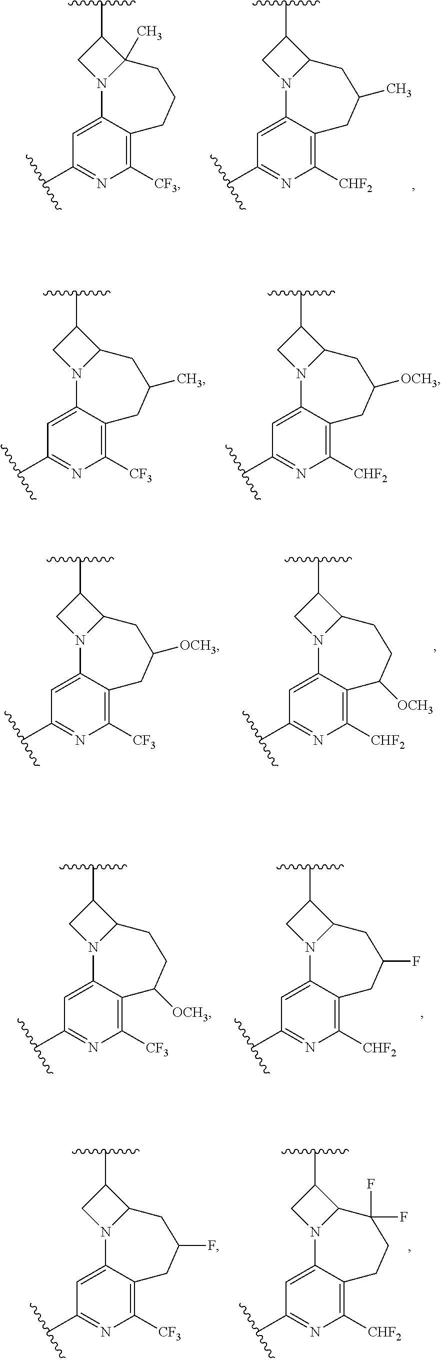

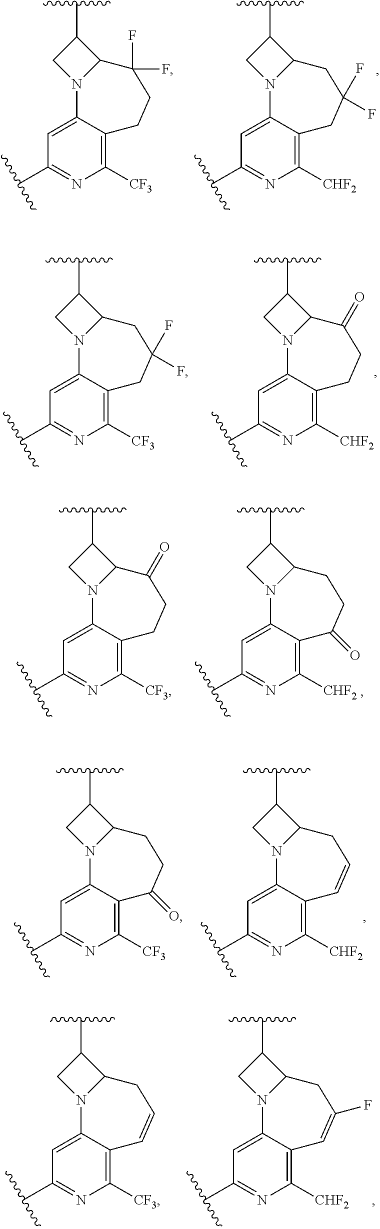

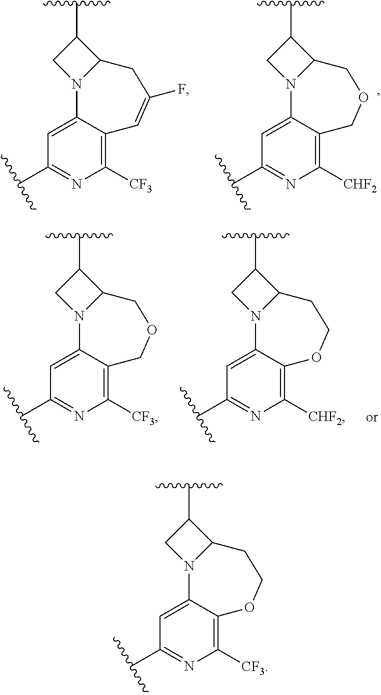 [SMILES: *c1cc2c(c(C(F)(F)F)n1)C=C(F)CC1C(*)CN21 ; *c1cc2c(c(C(F)F)n1)COCC1C(*)CN21 ; *c1cc2c(c(C(F)(F)F)n1)COCC1C(*)CN21 ; *c1cc2c(c(C(F)F)n1)OCCC1C(*)CN21 ; *c1cc2c(c(C(F)(F)F)n1)OCCC1C(*)CN21]

84. The compound of claim 83, wherein 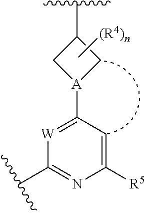
is

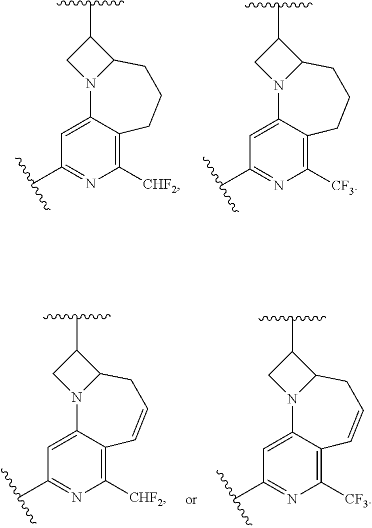 [SMILES: *c1cc2c(c(C(F)F)n1)CCCC1C(*)CN21 ; *c1cc2c(c(C(F)(F)F)n1)CCCC1C(*)CN21 ; *c1cc2c(c(C(F)F)n1)C=CCC1C(*)CN21 ; *c1cc2c(c(C(F)(F)F)n1)C=CCC1C(*)CN21]

85. The compound or salt of any one of claims 43-84, wherein: X is 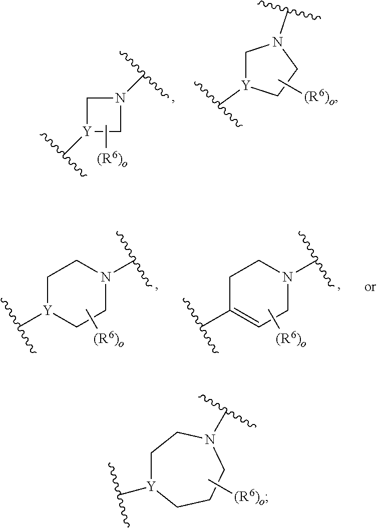
Y is N, C—H, C-halo, C—CN, C—C₁₋₃alkyl, C—C₁₋₃haloalkyl, C—C₀₋₂alkyleneOH, or C—C₀₋₃alkylene-C₁₋₄alkoxy;
o is 0, 1, 2, 3, or 4; and
each R⁶ independently is halo, CN, C₁₋₃alkyl, C₂₋₃alkenyl, C₁₋₃haloalkyl, C₀₋₃alkylene-OH, C₀₋₃alkylene-C₁₋₃alkoxy, deuterated C₀₋₆alkylene-C₁₋₃alkoxy, or C₁₋₄alkylene-N(R^{N1})₂; or two geminal R⁶, together with the atom to which they are attached, form oxo, =CH₂, spiro-C₃₋₇cycloalkyl, spiro-C₄₋₇cycloalkenyl, spiro-heterocycloalkyl having 4-7 total ring atoms and 1 or 2 heteroatoms selected from N, O and S, or spiro-heterocycloalkenyl having 4-7 total ring atoms and 1 or 2 heteroatoms selected from N, O and S; or two vicinal R⁶, together with the atoms to which they are attached, form fused-C₃₋₇cycloalkyl, fused-C₄₋₇cycloalkenyl, fused-heterocycloalkyl having 4-7 total ring atoms and 1-3 heteroatoms selected from N, O, and S, or fused-heterocycloalkenyl having 4-7 total ring atoms and 1 or 2 heteroatoms selected from N, O, and S; or two non-neighboring Rejoin together to form a C₁₋₃alkylene bridge, a C₂₋₃alkenylene bridge, a C₁₋₃ether bridge, or a C₁₋₃thioether bridge; or Y and a vicinal R⁶, together with the atoms to which they are attached, form fused-C₃₋₇cycloalkyl, fused-C₄₋₇cycloalkenyl, fused-heterocycloalkyl having 3-7 total ring atoms and 1-3 heteroatoms selected from N, O, and S, or fused-heterocycloalkenyl having 4-7 total ring atoms and 1 or 2 heteroatoms selected from N, O, and S; wherein the cycloalkyl, cycloalkenyl, heterocycloalkyl or heterocycloalkenyl of any of the foregoing is unsubstituted or substituted with 1-4 substituents, and each substituent independently is halo, C₁₋₃alkyl, C₁₋₄haloalkyl, C₀₋₂alkyleneOH, C₀₋₂alkyleneC₁₋₃alkoxy, or C₀₋₂alkyleneCN; and
each R^{N1} independently is H or C₁₋₃alkyl.

86. The compound or salt of claim 85, wherein X is

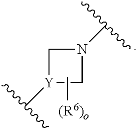

87. The compound or salt of claim 85, wherein X is

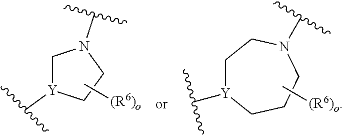

88. The compound or salt of claim 85, wherein X is

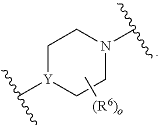

89. The compound or salt of claim 85, wherein X is

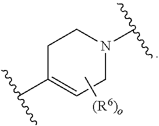

90. The compound or salt of any one of claims 85-88, wherein Y is N.

91. The compound or salt of any one of claims 85-88, wherein Y is CH.

92. The compound or salt of claim 85-88, wherein Y is C—F, C—Cl, C₂CH₃, C—CH₂CH₃, C—CH₂F, C—CHF₂, C—CF₃, C—OH, C—CH₂OH, C—OCH₃, or C—CH₂OCH₃.

93. The compound or salt of any one of claims 85-92, wherein o is 0.

94. The compound or salt of any one of claims 85-92, wherein o is 1.

95. The compound or salt of any one of claims 85-92, wherein o is 2.

96. The compound or salt of any one of claims 85-92 and 94-95, wherein each R⁶ independently is Br, Cl, F, CN, CH₃, CH₂F, CHF₂, CF₃, OH, CH₂OH, OCH₃, OCD₃, CH₂OCH₃, or CH₂N(CH₃)₂, or two geminal R⁶, together with the atom to which they are attached, form oxo, ═CH₂, spiro-cyclopropyl, spiro-cyclobutyl, spiro-oxetanyl, or spiro-tetrahydrofuranyl, or two vicinal R⁶, together with the atoms to which they are attached, form fused-cyclopropyl, fused-cyclobutyl, or fused-cyclopentyl, and any of the foregoing spiro and fused rings independently is unsubstituted or substituted with 1 or 2 substituents, and each substituent independently is halo, C₁₋₃alkyl, C₁₋₃haloalkyl, C₀₋₂alkyleneOH, C₀₋₂alkyleneC₁₋₃alkoxy, or C₀₋₂alkyleneCN.

97. The compound or salt of claim 96, wherein each substituent independently is F, Cl, OH, OCH₃, OCH₂CH₃, or CN.

98. The compound or salt of any one of claims 85-92, wherein two non-neighboring R⁶ join together to form a C₁₋₃alkylene bridge, a C₂₋₃alkenylene bridge, a C₁₋₃ether bridge, or a C₁₋₃thioether bridge.

99. The compound or salt of claim 98, wherein two non-neighboring R⁶ join together to form —CH₂—, —CH₂CH₂—, —CH₂CH₂CH₂—, —CH₂—CH—CH— or —CH₂OCH₂—.

100. The compound or salt of any one of claims 43-85, wherein X is

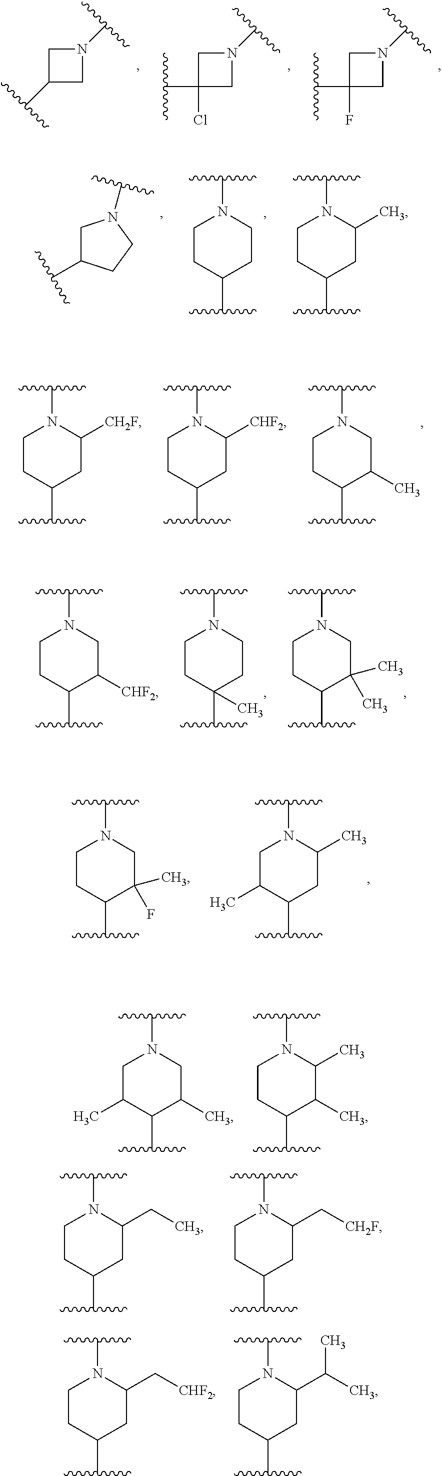

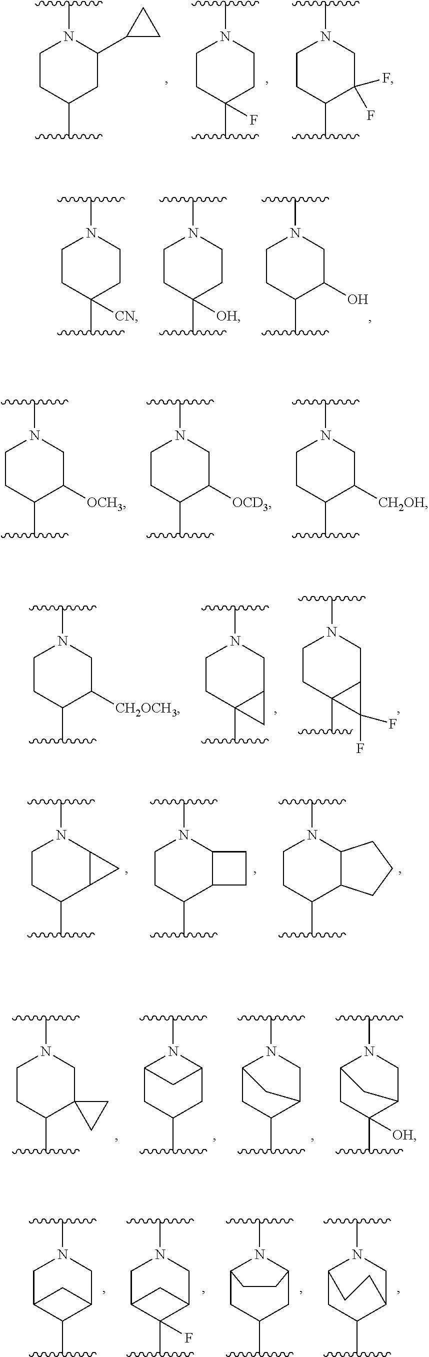

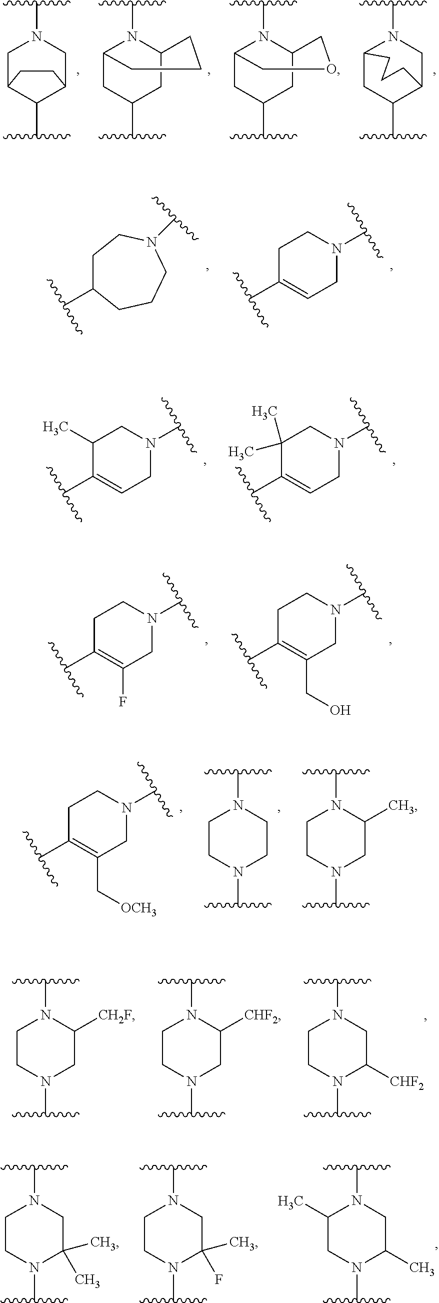

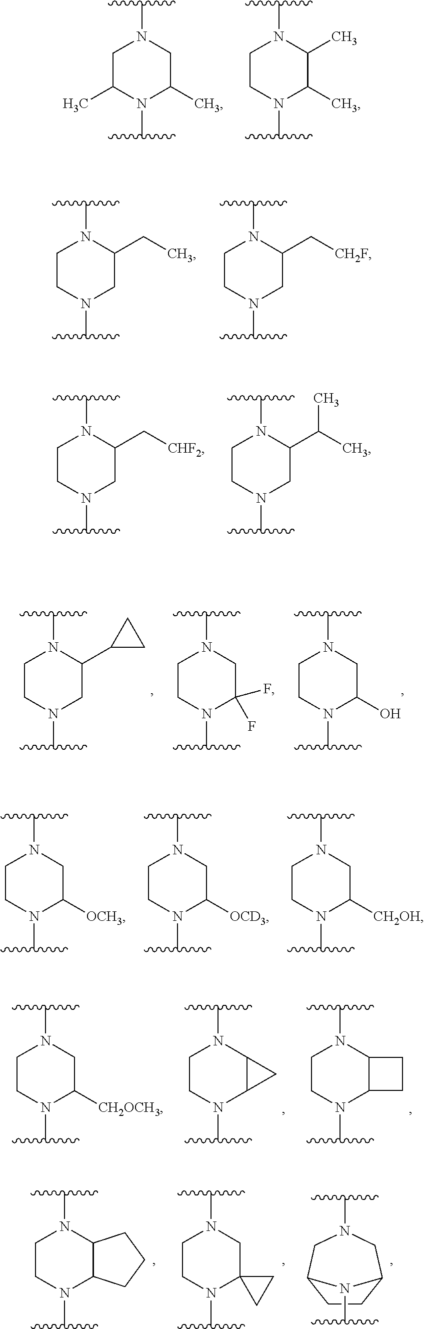

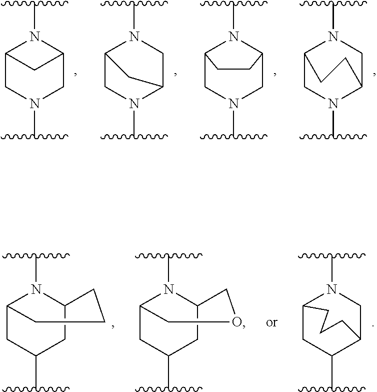

101. The compound or salt of claim 100, wherein X is

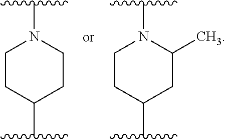 [SMILES: *C1CCN(*)CC1 ; *C1CCN(*)C(C)C1]

102. The compound or salt of any one of claims 43-101, wherein Z is unsubstituted phenyl or phenyl substituted with 1-4 substituents, and each substituent independently is halo, C₀₋₂alkyleneCN, C₀₋₂alkyleneOH, C₀₋₃alkylene-C₁₋₄alkoxy, C₀₋₃alkylene-C₁₄thioalkoxy, or 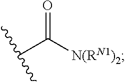
and each R^{N1} independently H or CH₃.

103. The compound or salt of claim 102, wherein each substituent independently is F, Cl, CN, OCH₃, SCH₃, CH₂OH, or

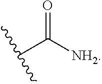 [SMILES: *C(N)=O]

104. The compound or salt of claim 102 or 103, wherein Z is

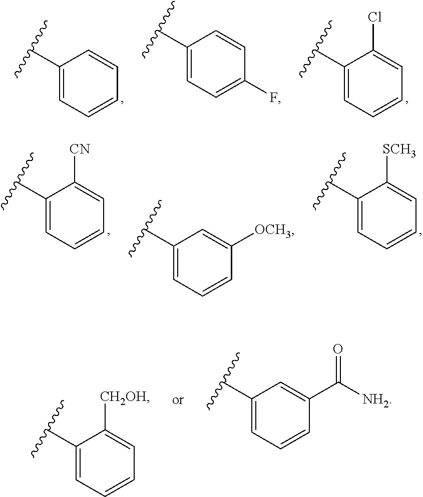

105. The compound or salt of any one of claims 43-101, wherein Z is heteroaryl comprising 5 or 6 total ring atoms and 1-3 heteroatoms selected from N, O, and S, wherein the heteroaryl is unsubstituted or substituted with 1-4 substituents, and each substituent independently is halo, CN, C₁₋₆alkyl, C₁₋₆haloalkyl, C₂₋₆alkenyl, C₂₋₆haloalkenyl, C₀₋₆alkylene-OH, C₀₋₆alkylene-C₁₋₃alkoxy, C₀₋₆alkylene-N(R^{N1})₂, C₀₋₂alkylene-C₃₋₆cycloalkyl, C₀₋₂alkylene-heterocycloalkyl having 3-6 total ring atoms and 1-3 heteroatoms selected from N, O, and S, or C₀₋₂alkylene-phenyl;
wherein each of the C₁₋₆alkyl, C₂₋₆alkenyl, C₀₋₆alkylene-C₁₋₃alkoxy, cycloalkyl, heterocycloalkyl, and phenyl substituents independently is unsubstituted or substituted with 1-3 further substituents, and each further substituent independently is D, halo, C₁₋₃alkyl, C₁₋₆haloalkyl, C₁₋₂alkyleneOH, C₁₋₂alkylene-C₁₋₃alkoxy, C₁₋₃deuterated alkoxy, N(R^{N1})₂, (C═O)C₁₋₃alkyl, C₃₋₅cycloalkyl, heterocycloalkyl having 3-5 total ring atoms and 1 or 2 heteroatoms selected from N, O, and S; or two geminal further substituents, together with the atom to which they are attached, form spiro-C₃₋₅cycloalkyl, or spiro-heterocycloalkyl having 3-5 total ring atoms and 1 or 2 heteroatoms selected from N, O, and S; or two vicinal further substituents, together with the atoms to which they are attached, form fused-C₃₋₅cycloalkyl or fused-heterocycloalkyl having 3-5 total ring atoms and 1 or 2 heteroatoms selected from N, O, and S; wherein each of the foregoing cycloalkyl and heterocycloalkyl further substituents independently is unsubstituted or substituted with 1 or 2 substituents, and each substituent independently is halo or C₁₋₃alkyl; and
each R^{N1} independently is H or C₁₋₃alkyl.

106. The compound or salt of claim 105, wherein the heteroaryl is pyrrolyl, furanyl, thiophenyl, pyrazolyl, imidazolyl, thiazolyl, isothiazolyl, oxazolyl, isoxazolyl, triazolyl, thiadiazolyl, oxadiazolyl, pyridyl, pyridazinyl, pyrimidinyl, pyrazinyl, or triazinyl.

107. The compound or salt of claim 106, wherein the heteroaryl is pyrazolyl or pyridyl.

108. The compound or salt of any one of claims 105-107, wherein the heteroaryl is substituted with 1 or 2 substituents.

109. The compound or salt of any one of claims 105-108, wherein each substituent independently is Br, Cl, F, CN, CF₃, CHF₂, CH₂F, CH₂CHF₂, CH₂CH₂F, CH(CH₂F)₂, CH(CH₃)CH₂F, CH(CH₃)CHF₂, C(═CH₂)CH₂F, OH, CH₂OH, CH₂CH₂OH, CH(CH₃)CH₂OH, C(CH₃)₂OH, C(CH₃): CH₂OH, CH₂C(CH₃)₂OH, NH₂, CH₂NH₂, CH₂NHCH₃, CH₂N(CH₃)₂, CH₂CH₂NH₂, CH₂CH₂NHCH₃, CH₂CH₂N(CH₃)₂, C₁₋₆alkyl selected from CH₃, CH₂CH₃, CH₂CH₂CH₃, and CH(CH₃)₂, C₂₋₆alkenyl selected from CH═CH₂, CH₂CH═CH₂, and CH═CHCH₃, C₀₋₆alkylene-C₁₋₃alkoxy selected from OCH₃, CH₂OCH₃, CH₂CH₂OCH₃, CH₂CH₂OCH₂CH₃, CH₂CH₂CH₂OCH₃, CH(CH))OCH₃, CH(CH₃)CH₂OCH₃, CH(OCH₃)CH₂OCH₃, CH(CH₃)(OCH₃)CH₂OCH₃, C(CH₃)₂OCH₃, C(CH₃)CH₂OCH₃, CH₂CH(CH₃)OCH₃, CH₂ (CH₃)(OCH₃)OCH₃, CH₂C(CH₃)₂OCH₃, and CH₂C(CH₃)₂OCH₃, cycloalkyl selected from cyclopropyl, cyclobutyl, cyclopentyl, and cyclohexyl, or heterocycloalkyl selected from azetidinyl, pyrrolidinyl, piperidinyl, pyrazolidinyl, oxetanyl, tetrahydrofuranyl, tetrahydropyranyl, isoxazolidinyl, and morpholinyl;
wherein each of the C₁₋₃alkyl, C₂₋₆alkenyl, C₀₋₃alkylene-C₁₋₃alkoxy, cycloalkyl, and heterocycloalkyl substituents independently is unsubstituted or substituted with 1-3 further substituents and each further substituent independently is D, halo, C₁₋₃alkyl, C₁₋₃haloalkyl, C₁₋₂alkyleneOH, C₁₋₂alkylene-C₁₋₃alkoxy, C₁₋₃deuterated alkoxy, N(R^{N1})₂, (C═O) C₁₋₃alkyl, C₃₋₅cycloalkyl, heterocycloalkyl having 3-5 total ring atoms and 1 or 2 heteroatoms selected from N, O, and S, or two geminal further substituents, together with the atom to which they are attached, form C₃₋₅spiro-cycloalkyl, or spiro-heterocycloalkyl having 3-5 total ring atoms and 1 or 2 heteroatoms selected from N, O, and S; or two vicinal further substituents, together with the atoms to which they are attached, form fused-C₃₋₅cycloalkyl or fused-heterocycloalkyl having 3-5 total ring atoms and 1 or 2 heteroatoms selected from N, O, and S.

110. The compound or salt of claim 109, wherein each further substituent independently is D, Br, Cl, F, OH, CH₃, CF₃, CF₂H, CFH₂, OCH₃, OCD₃, CH₂OCH₃, N(CH₃)₂, (C═O)CH₃, oxetanyl, or azetidinyl, or two geminal further substituents, together with the atom to which they are attached, form spiro-oxetanyl or spiro-azetidinyl; wherein each of the foregoing oxetanyl, azetidinyl, spiro-oxetanyl, and spiro-azetidinyl independently is unsubstituted or substituted with F, CH₃, or a combination thereof

111. The compound or salt of claim 110, wherein each further substituent independently is D, Br, Cl, F, OH, CH₃, CF₃, CF₂H, CFH₂, OCH₃, OCD₃, N(CH₃)₂, (C═O)CH₃, 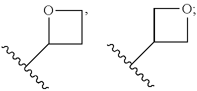 [SMILES: *C1CCO1 ; *C1COC1]
or two geminal further substituents, together with the atom to which they are attached, form

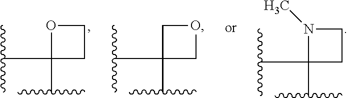

112. The compound or salt of any one of claims 105-108, wherein each substituent of the heteroaryl of Z independently is CI, F, CN, CH₃, CD₃, CH₂CH₃, CH(CH₃)₂, CF₃, CHF₂, CH₂F, CH₂CHF₂, CH₂CH₂F, CH(CH₂F)₂, CH(CH₃)CH₂F, CH(CH₃)CHF₂, C(═CH₂)CH₂F, OH, CH₂OH, CH—CH₂OH, CH(CH₃)CH₂OH, C(CH₃)₂OH, C(CH₃)₂CH₂OH, CH₂C(CH₃)₂OH, OCH₃, OCD₃, CH₂OCH₃, CH₂OCD₃, CH₂CH₂OCH₃, CHFCH₂OCH₃, CF₂CH₂OCH₃, CH₂CH₂OCD₃, CH₂CH₂OCH₂CH₃, CH₂CH₂CH₂OCH₃, CH₂CH₂CH₂OCD₃, CH(CH₃)OCH₃, CH(CH₃)CH₂OCH₃, CH(OCH₃)CH₂OCH₃, CH(CH₃)(OCH₃)CH₂OCH₃, CH(CH₂F) (CH₃)CH₂OCD₃, CH(CH₃)CH₂OCD₃, C(CH₃)₂OCH₃, C(CH₃)₂CH₂OCH₃, C(CH₃)₂CH₂OCD₃, CH₂CH(CH₃)OCH₃, CH₂(CH₃)(OCH₃)OCH₃, CH₂CH(CH₃)OCD₃, CH₂C(CH₃)₂OCH₃, CH₂C(CH₃)₂OCD₃, NH₂, CH₂NH₂, CH₂NHCH₃, CH₂N(CH₃)₂, CH₂CH₂NH₂, CH₂CH₂NHCH₃, CH₂CH₂N(CH₃)₂,

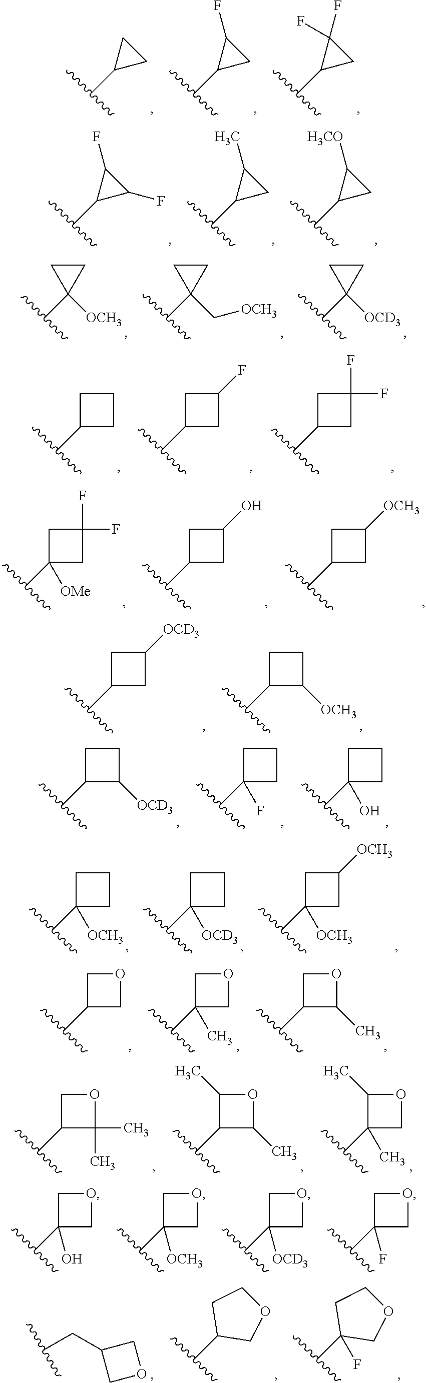

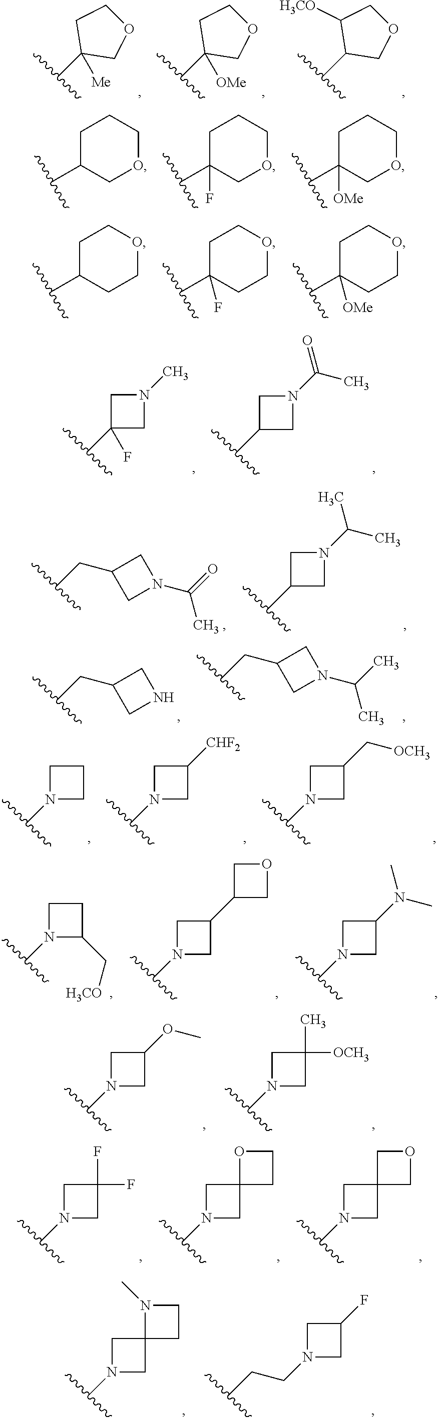

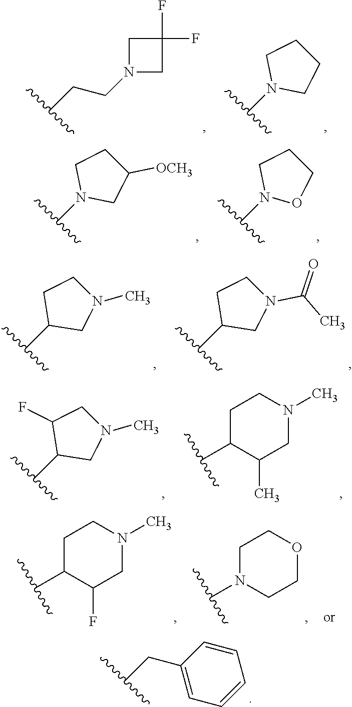

113. The compound or salt of claim 112, wherein each substituent of the heteroaryl 1 of Z independently is CH₃, C(CH₃)₂CH₂OH, CH₂CH₂OCH₃, CH₂CH₂OCD₃, CH(CH₃)OCH₃,

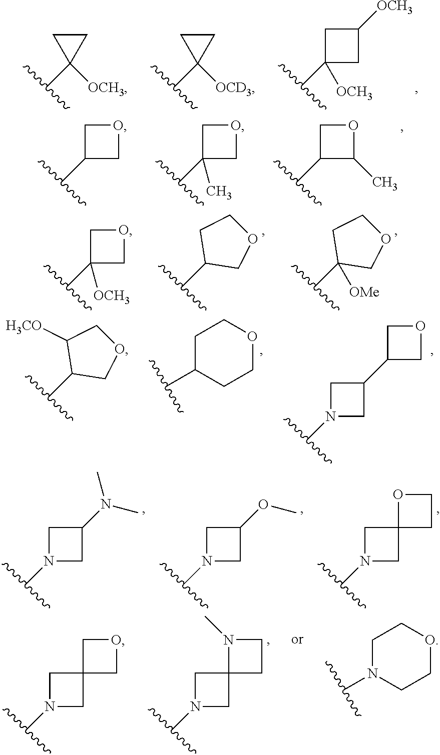

114. The compound or salt of claim 113, wherein each substituent of the heteroaryl 1 of Z independently is CH₃, CH₂CH₂OCH₃, 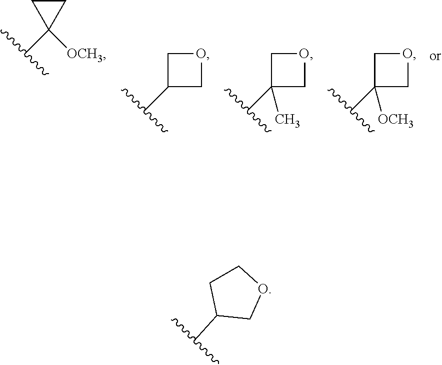
or any combination of the foregoing.

115. The compound or salt of any one of claims 43-105, wherein Z is

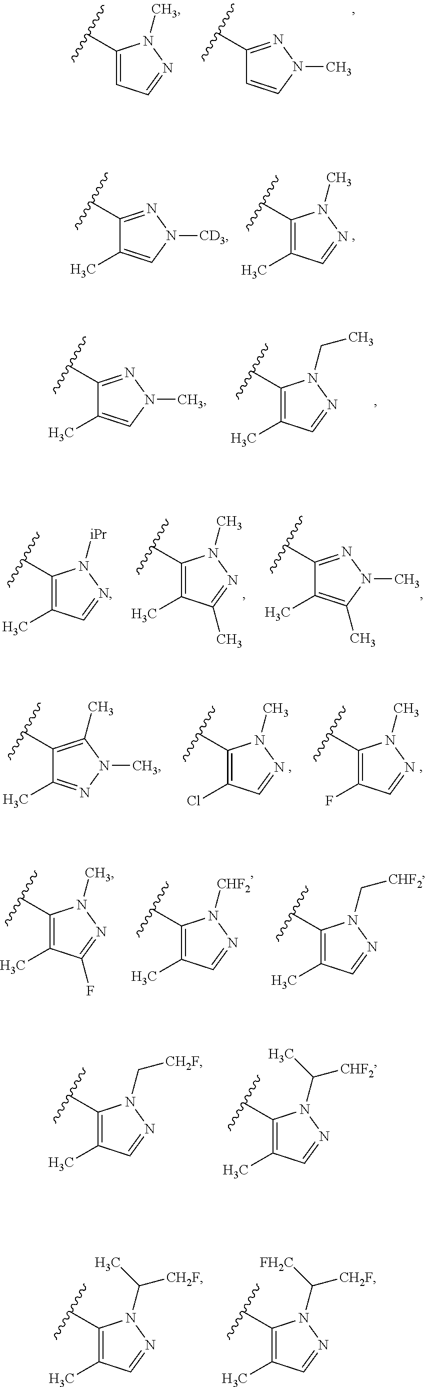

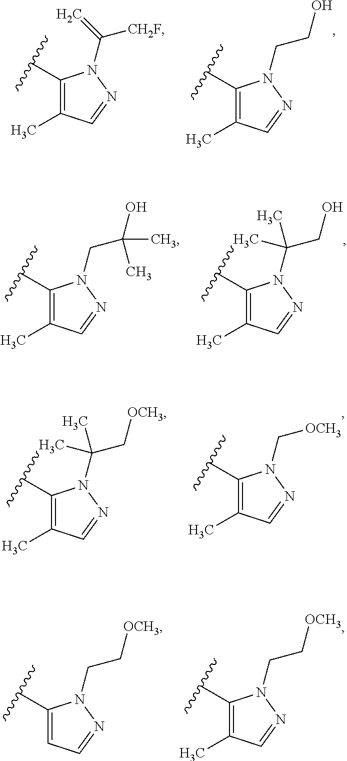

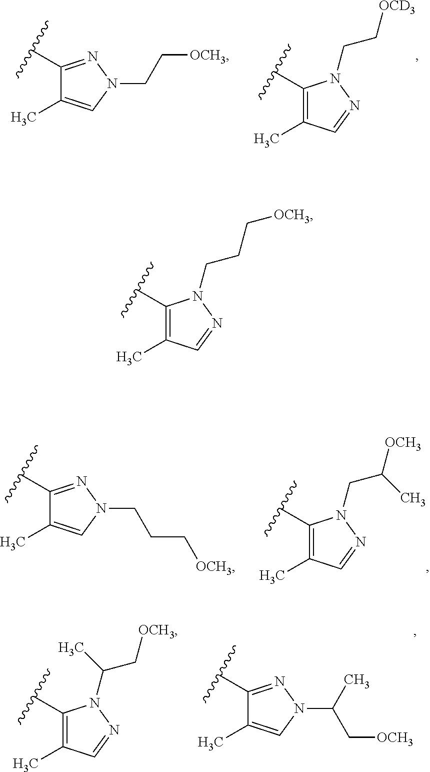

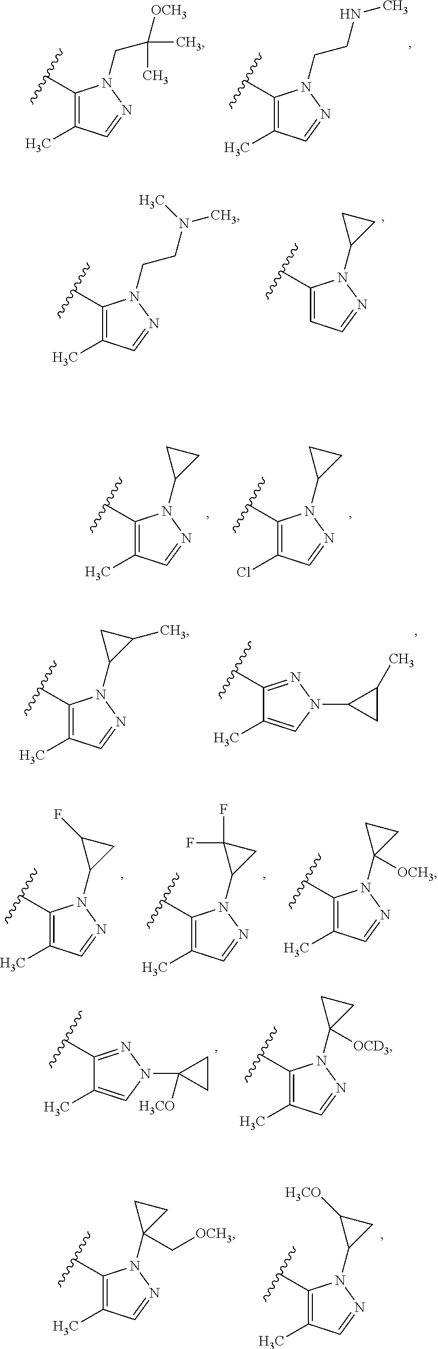

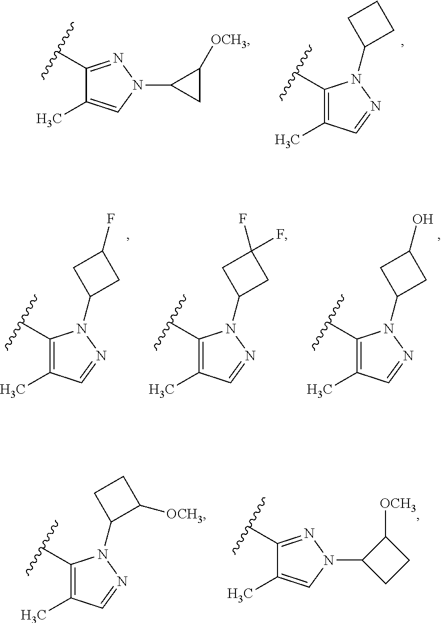

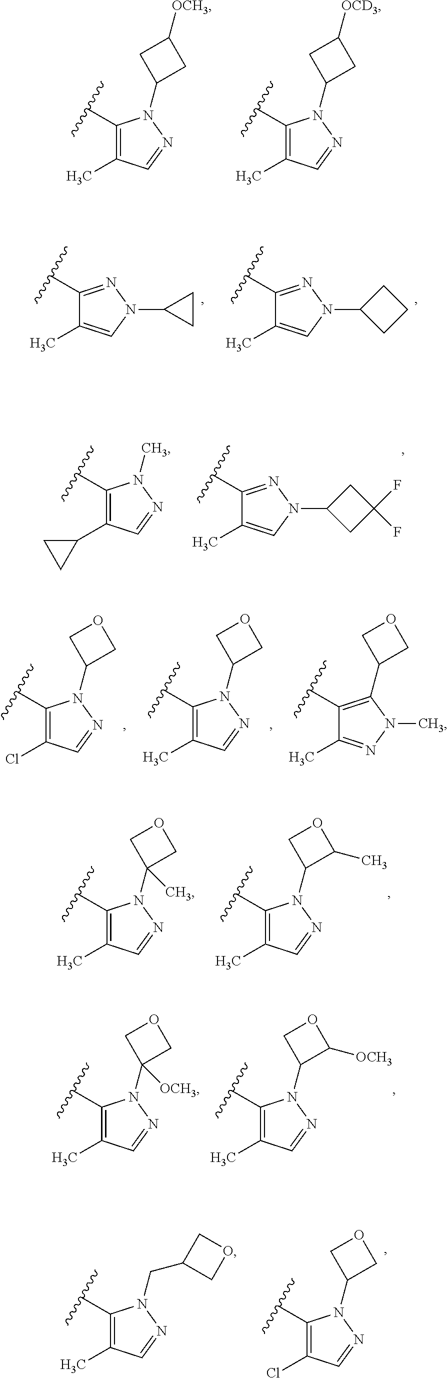

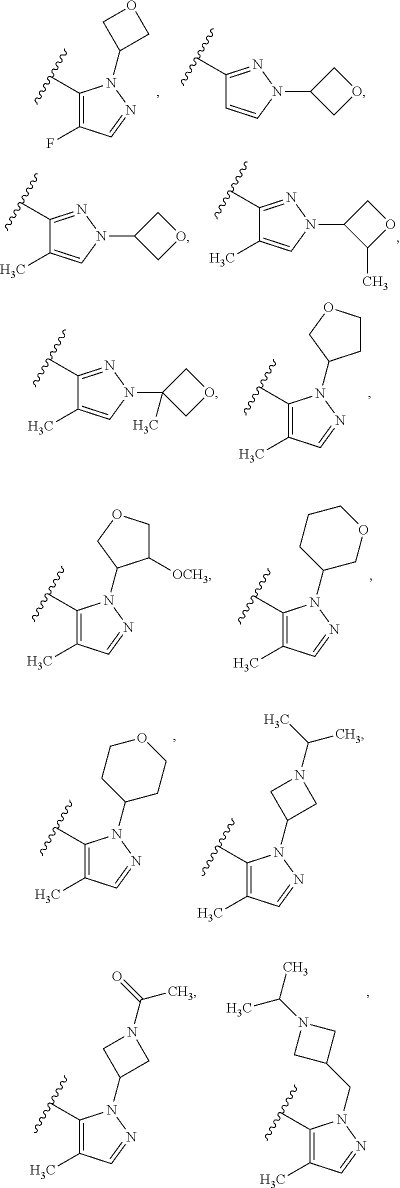

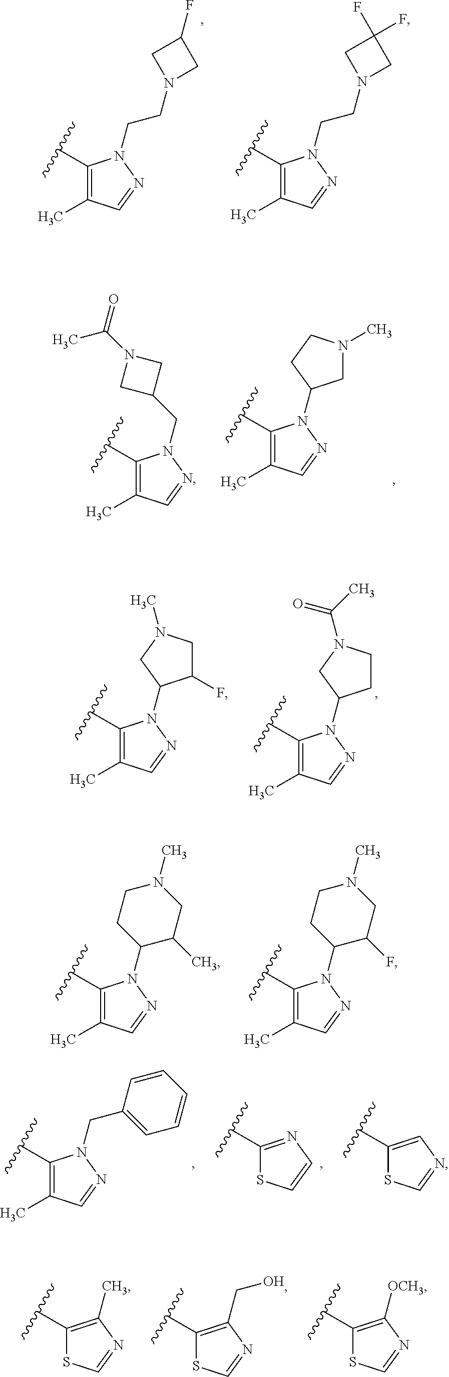

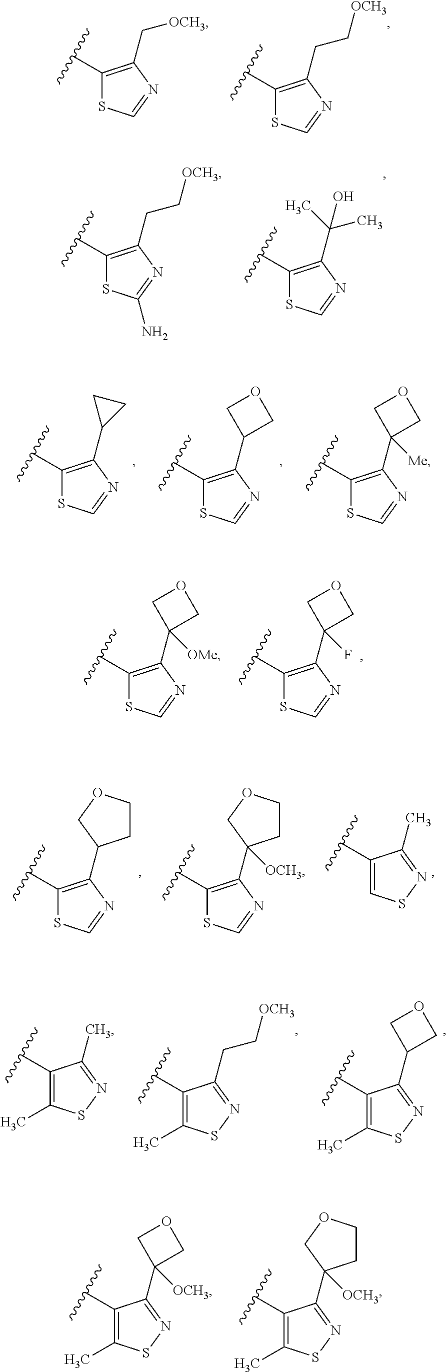

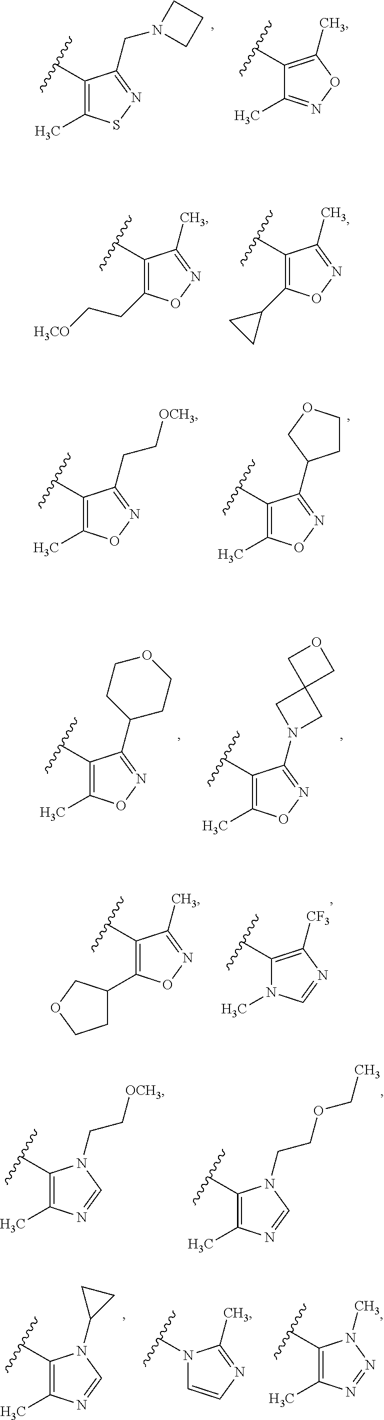

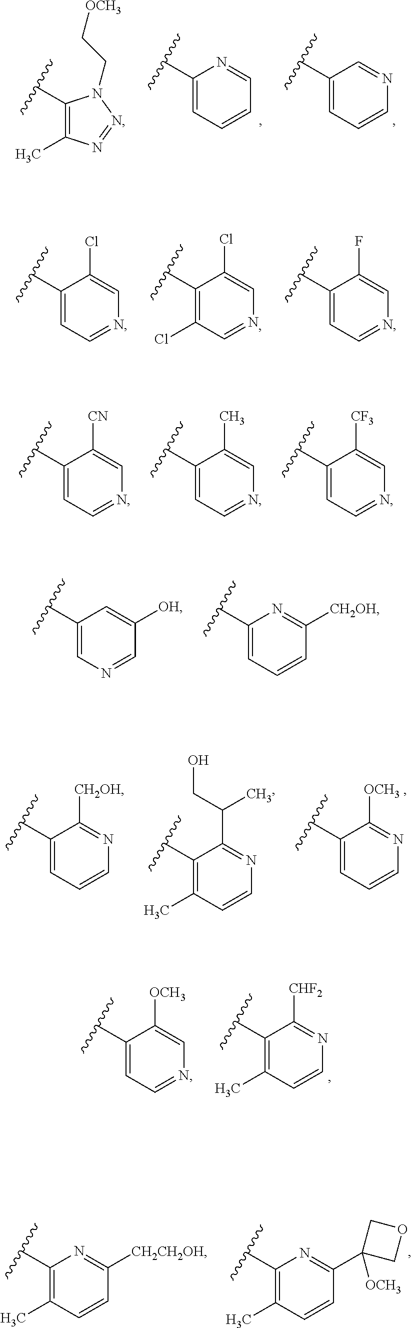

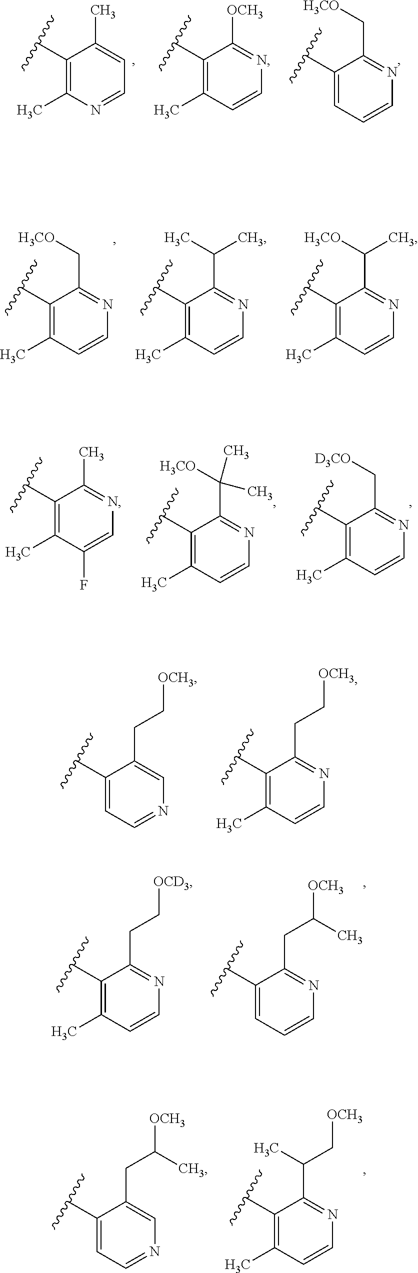

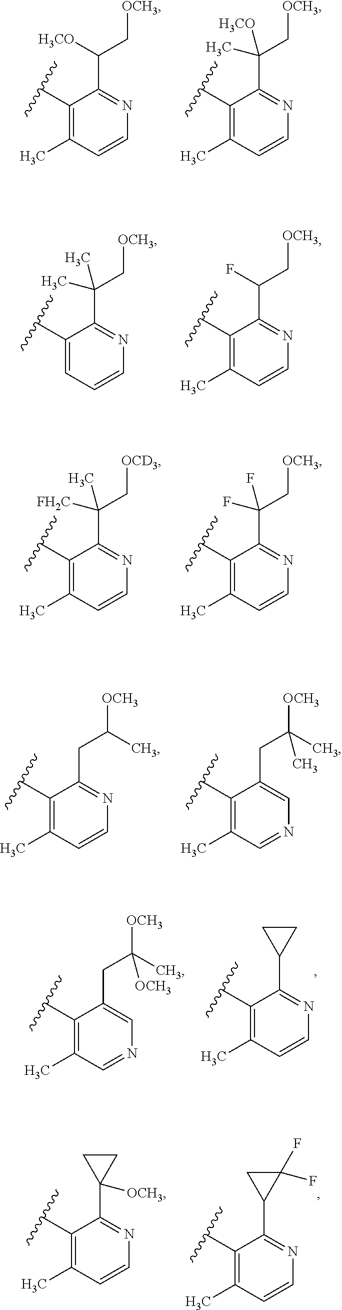

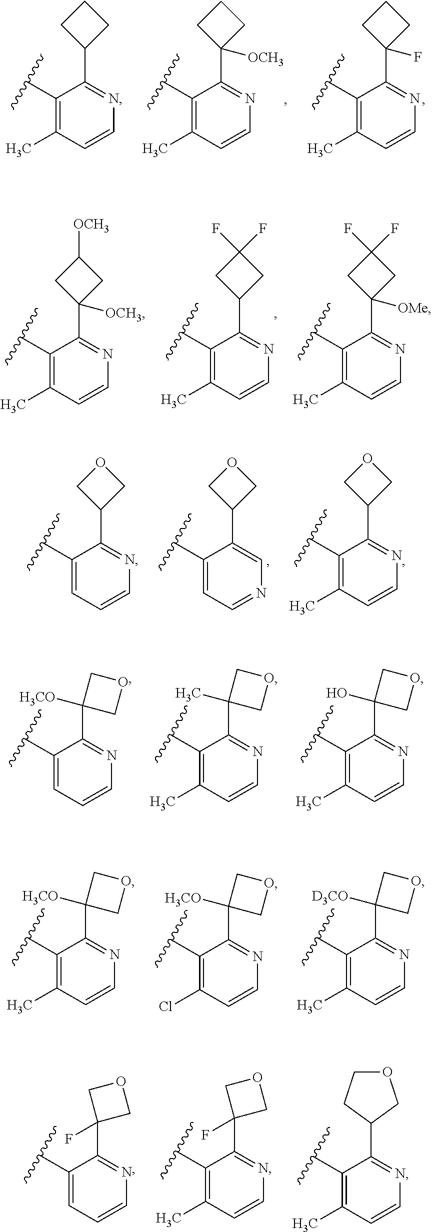

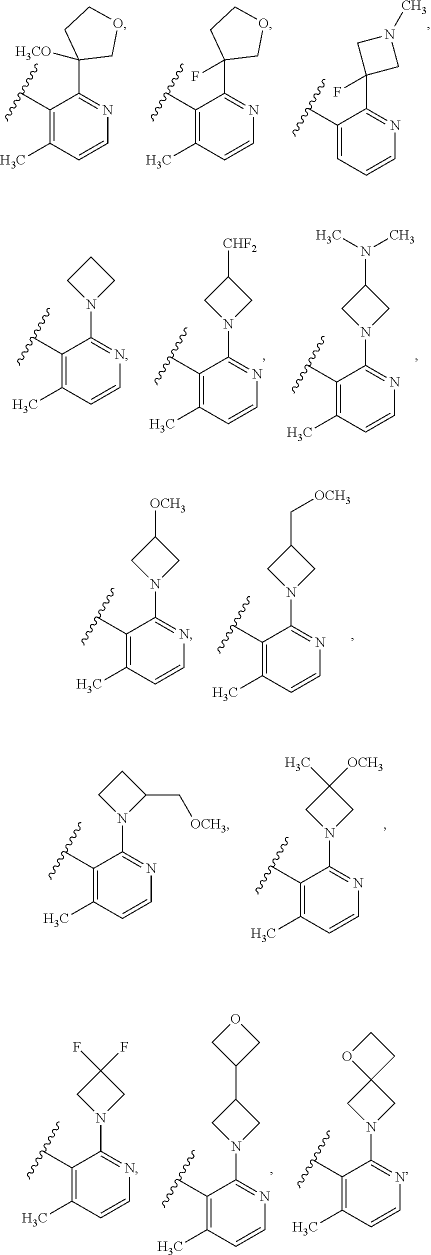

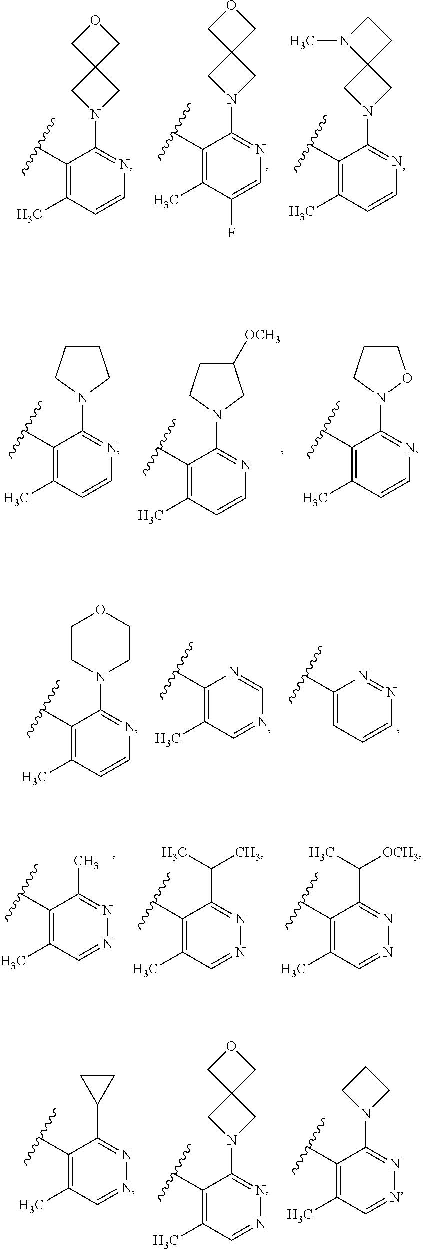

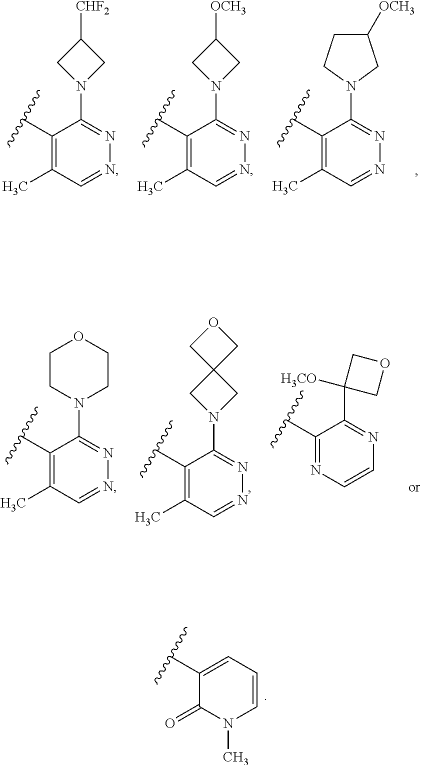

116. The compound or salt of claim 115, wherein Z is

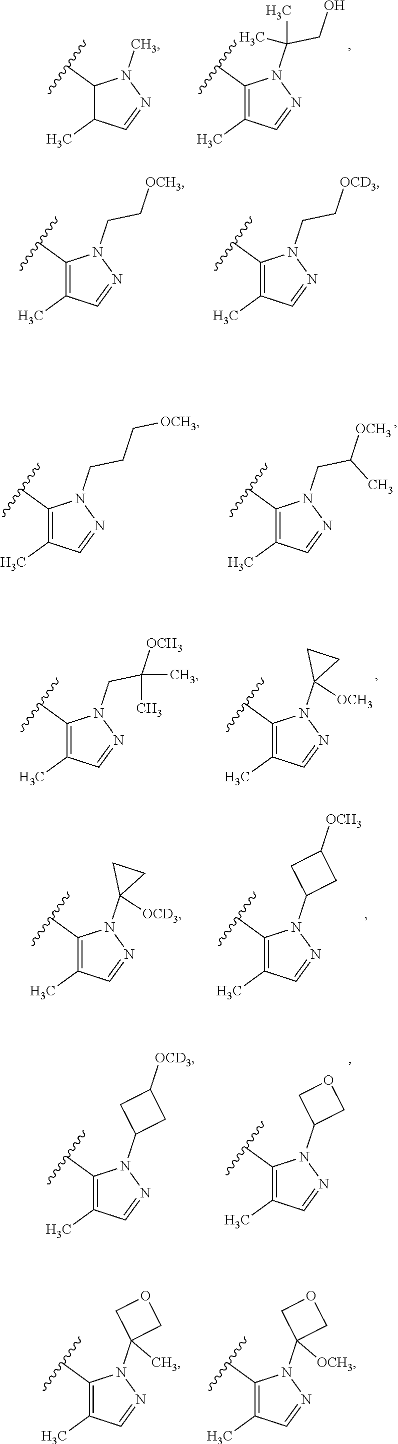

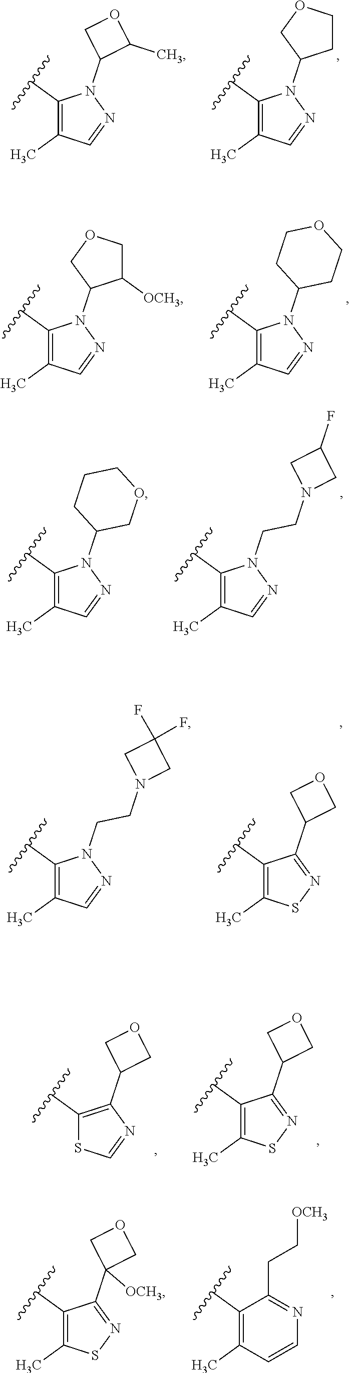

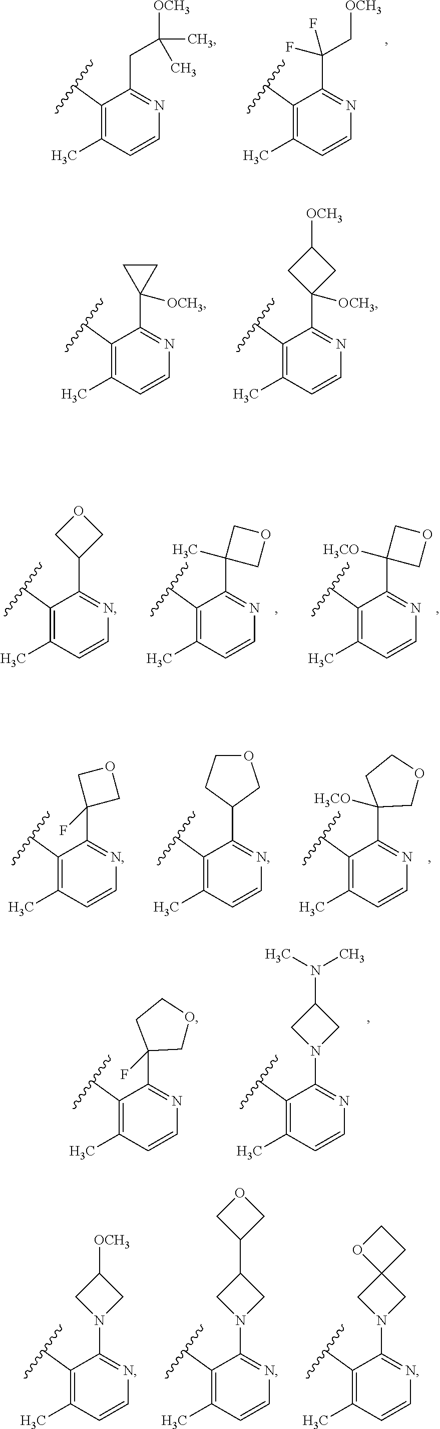

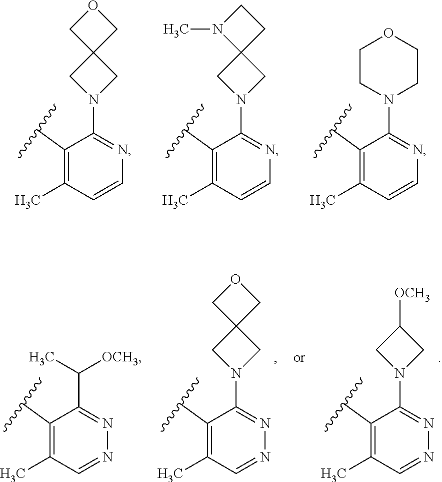 [SMILES: *c1c(C)ccnc1N1CC2(COC2)C1 ; *c1c(C)ccnc1N1CC2(CCN2C)C1 ; *c1c(C)ccnc1N1CCOCC1 ; *c1c(C)cnnc1C(C)OC ; *c1c(C)cnnc1N1CC2(COC2)C1 ; *c1c(C)cnnc1N1CC(OC)C1]

117. The compound or salt of claim 116, wherein Z is

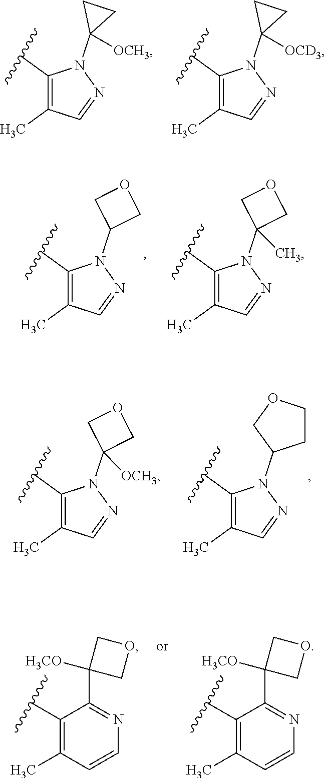

118. The compound or salt of any one of claims 43-101, wherein Z is a bicyclic ring comprising heteroaryl having 5 or 6 total ring atoms and 1-3 heteroatoms selected from N, O, and S fused to C₅₋₆cycloalkyl or a heterocycloalkyl ring having 5 or 6 total ring atoms and 1 or 2 heteroatoms selected from N, O, and S; wherein the bicyclic ring is unsubstituted or substituted with 1-4 substituents, and each substituent independently is halo, CN, C₁₋₆alkyl, C₁₋₆haloalkyl, C₀₋₆alkylene-OH, or C₀₋₆alkylene-C₁₋₃alkoxy.

119. The compound or salt of claim 118, wherein Z is

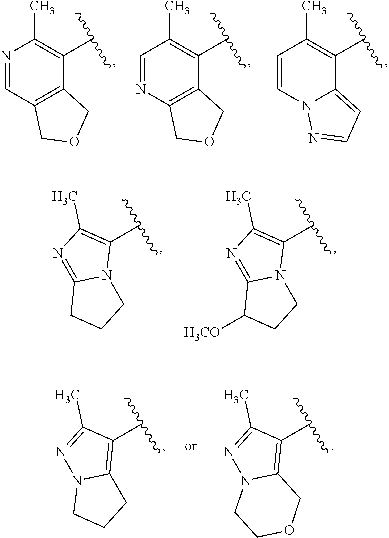

120. The compound or salt of claim 43, wherein: 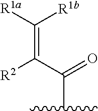
is 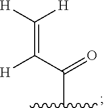 [SMILES: *C(=O)C=C]
is 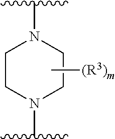
is 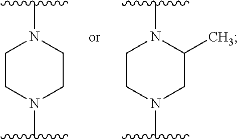 [SMILES: *N1CCN(*)CC1 ; *N1CCN(*)C(C)C1] 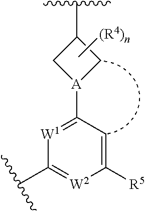
is 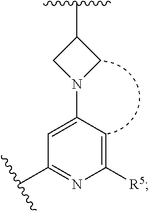
R⁵ is CHF, or CF₃;
X is 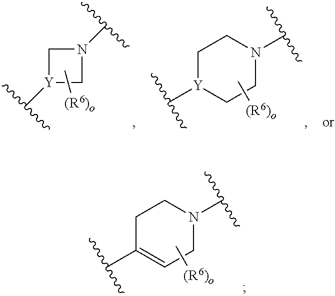
is 0 or 1;
R⁶ is CH₃;
Y is CH or N; and
Z is pyrazolyl or pyridyl, each of which is substituted with 1 or 2 substituents, and each substituent independently is Cl, F, CN, CH₃, CD₃, CH₂CH₃, CH(CH₃)₂, CF₃, CHF₂, CH₂F, CH₂CHF₂, CH₂CH₂F, CH(CH₂F)₂, CH(CH₃)CH₂F, CH(CH₃)CHF₂, C(═CH₂)CH₂F, OH, CH₂OH, CH₂CH₂OH, CH(CH₃)CH₂OH, C(CH₃)₂OH, C(CH₃)CH₂OH, CH₂C(CH₃)₂OH, OCH₃, OCD₃, CH₂OCH₃, CH₂OCD₃, CH₂CH₂OCH₃, CHFCH₂OCH₃, CF₂CH₂OCH₃, CH₂CH₂OCD₃, CH₂CH₂OCH₂CH₃, CH₂CH₂CH₂OCH₃, CH₂CH₂CH₂OCD₃, CH(CH₃)OCH₃, CH(CH)CH₂OCH₃, CH(OCH₃)CH₂OCH₃, CH(CH₃)(OCH,)CH₂OCH₃, CH(CH₂F) (CH₃)CH₂OCD₃, CH(CH₃)CH₂OCD₃, C(CH₃)₂OCH₃, C(CH₃)₂CH₂OCH₃, C(CH₃)₂CH₂OCD₃, CH₂CH(CH₃)OCH₃, CH(CH₃)(OCH₃)OCH₃, CH₂CH(CH₃)OCD₃, CH₂C(CH₃)₂OCH₃, CH₂C(CH₃)₂OCD₃, NH₂, CH₂NH₂, CH₂NHCH₃, CH₂N(CH₃)₂, CH₂CH₂NH₂, CH₂CH₂NHCH₃, CH₂CH₂N(CH₃)₂,

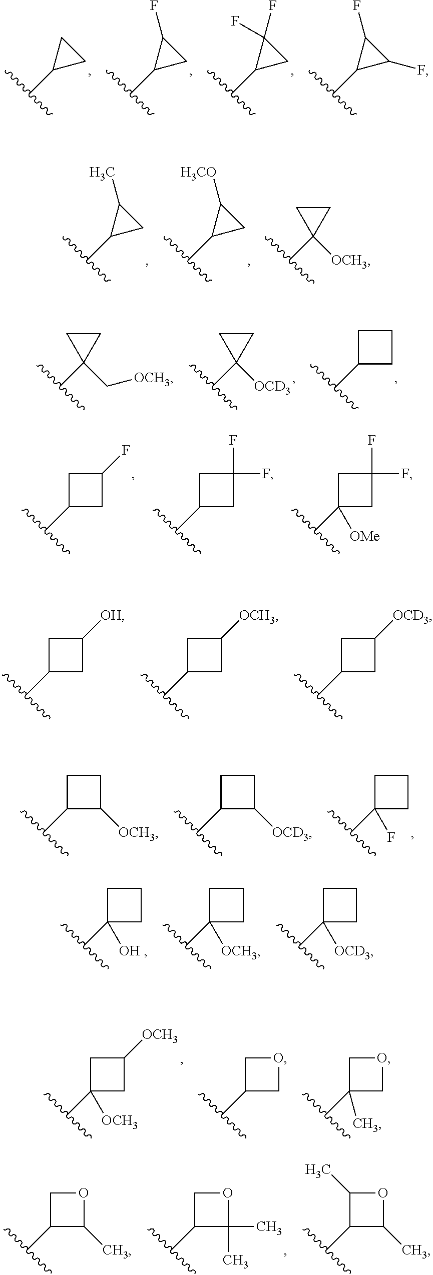

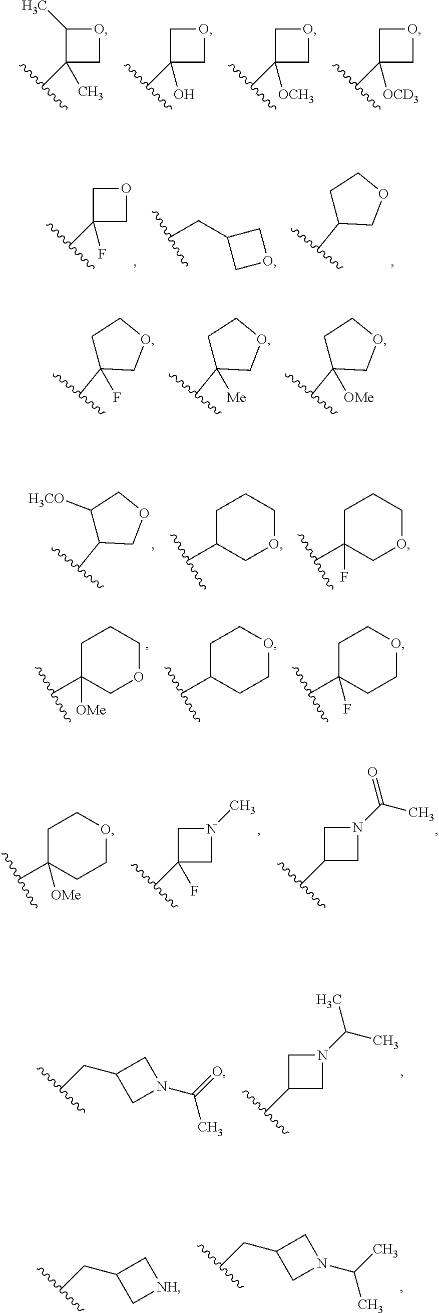

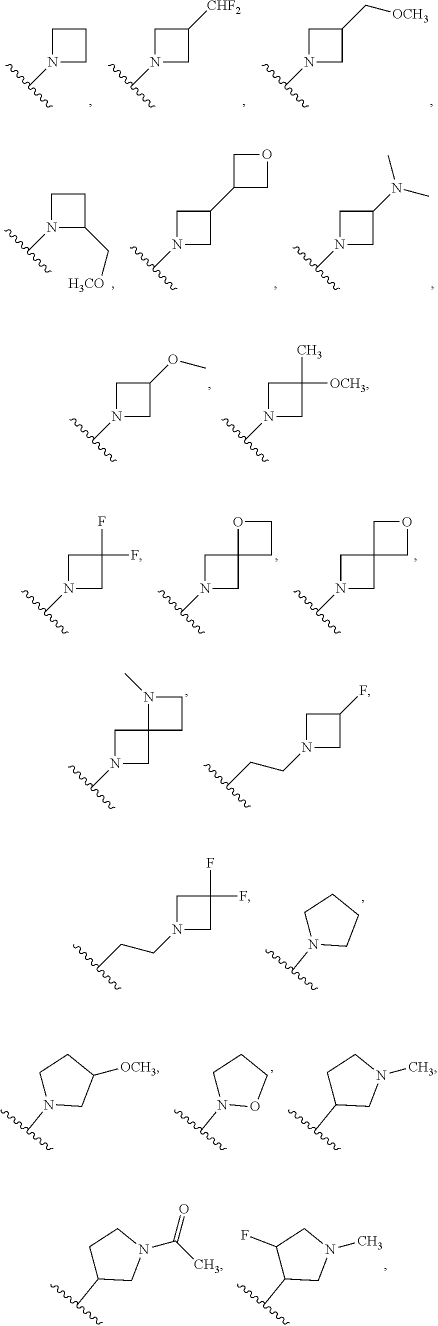

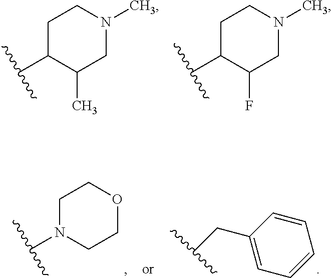

121. The compound or salt of claim 120, wherein X is

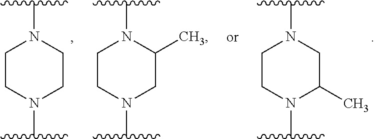

122. The compound or salt of claim 120 or 121, wherein [symbol] is

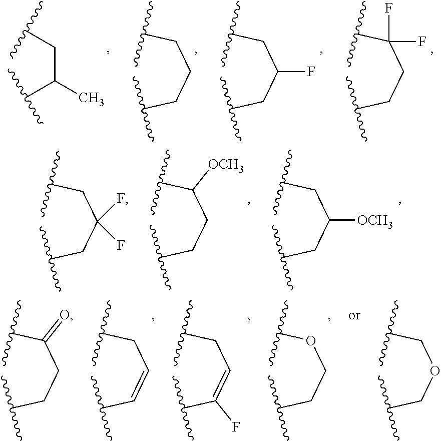

123. The compound or salt of claim 122, wherein [symbol] is

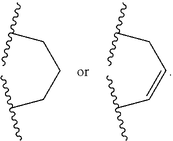 [SMILES: *CCC* ; */C=C\C*]

124. The compound or salt of any one of claims 120-123, wherein Z is

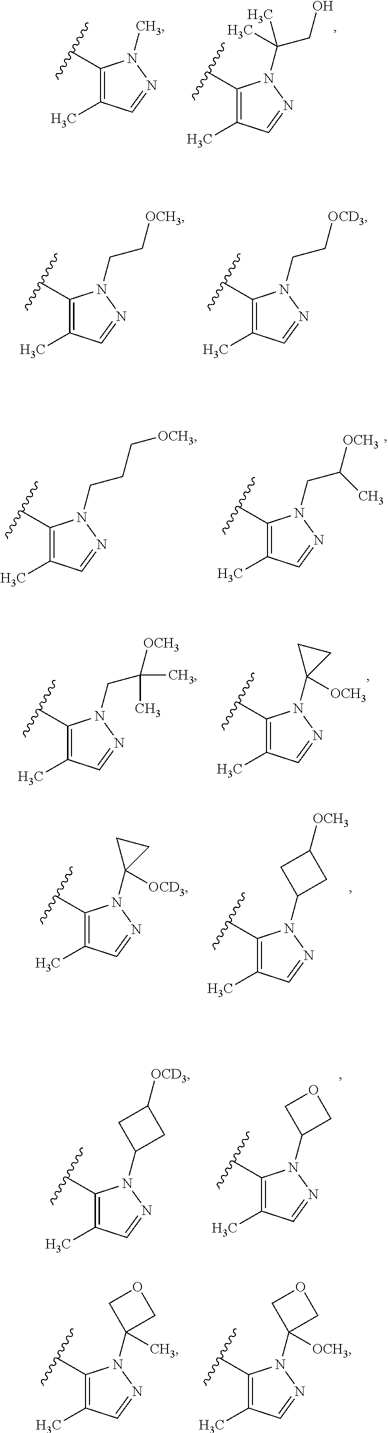

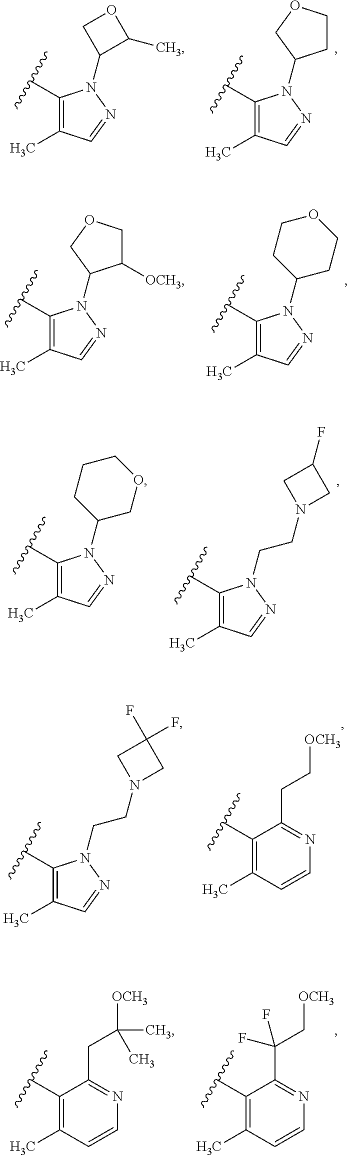

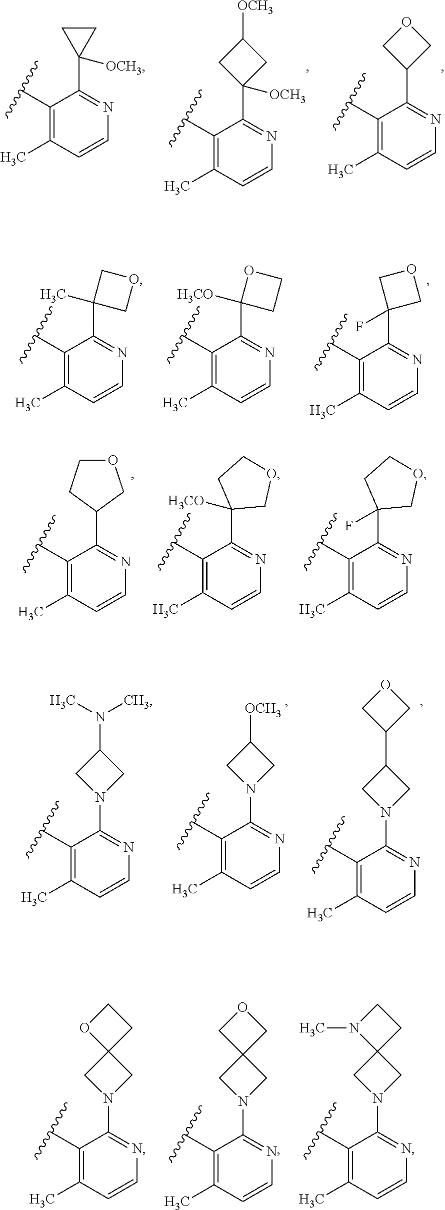

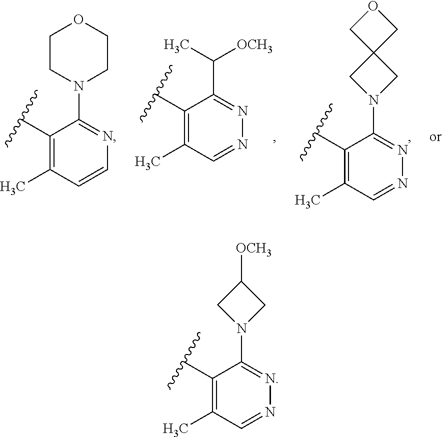 [SMILES: *c1c(C)ccnc1N1CCOCC1 ; *c1c(C)cnnc1C(C)OC ; *c1c(C)cnnc1N1CC2(COC2)C1 ; *c1c(C)cnnc1N1CC(OC)C1]

125. The compound or salt of claim 124, wherein Z is

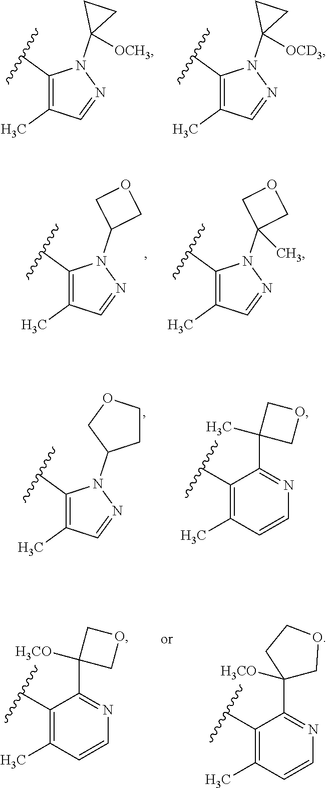

126. The compound of claim 43, wherein the compound is a compound listed in Table A, or a pharmaceutically acceptable salt thereof.

127. The compound of claim 126, wherein the compound is a compound listed in Table B, or a pharmaceutically acceptable salt thereof.

128. The compound of claim 43, wherein the compound is a compound listed in Table A′, or a pharmaceutically acceptable salt thereof.

129. The compound of claim 128, wherein the compound is a compound listed in Table B′ or a pharmaceutically acceptable salt thereof.

130. A pharmaceutical composition comprising the compound or salt of any one of claims 1-129 and a pharmaceutically acceptable excipient.

131. A method of treating cancer in a subject in need of treatment, the method comprising administering to the subject a therapeutically effective amount of the compound or salt of any one of claims 1-129, or the composition of claim 130.

132. The method of claim 131, wherein one or more cancer cells express KRAS G12C mutant protein.

133. The method of claim 131 or 132, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, a solid tumor, or any combination of the foregoing.

134. The method of claim 324, wherein the cancer is non-small cell lung cancer, colorectal cancer, pancreatic cancer, or solid tumor.

135. The method according to any one of claims 131-134, wherein the subject has a cancer that was determined to have one or more cells expressing the KRAS G12C mutant protein prior to administration of the compound, salt, or pharmaceutical composition.

136. The method according to any one of claims 131-134, further comprising simultaneous, separate, or sequential administration of an effective amount of a second compound, wherein the second compound is an ATR inhibitor, Aurora kinase A inhibitor, AKT inhibitor, arginase inhibitor, CDK2 inhibitor, CDK4/6 inhibitor, ErbB family inhibitor, ERK inhibitor, FAK inhibitor, FGFR inhibitor, glutaminase inhibitor, IGF-IR inhibitor, KIF18A inhibitor, MAT2A inhibitor, MCL-1 inhibitor, MEK inhibitor, mTOR inhibitor, PARP inhibitor, PD-1 inhibitor, PD-L1 inhibitor, PI3K inhibitor, PRMT5 inhibitor, Raf kinase inhibitor, SHP2 inhibitor, SOSI inhibitor, Src kinase inhibitor, or one or more chemotherapeutic agents.

137. The compound or salt of any one of claims 1-129, or the composition of claim 130 for use as a medicament.

138. The compound or salt of any one of claims 1-129, or the composition of claim 130 for use in treating cancer.

139. The compound or salt of any one of claims 1-129 or the pharmaceutical composition of claim 130 for use in treating cancer, wherein one or more cancer cells express KRAS G12C mutant protein.

140. The compound or salt of claim 138 or 139, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, or a solid tumor.

141. Use of a compound or salt of any one of claims 1-129 or the pharmaceutical composition of claim 130 for the manufacture of a medicament for the treatment of cancer.

142. Use of a compound or salt of any one of claims 1-129 or the pharmaceutical composition of claim 130 in the preparation of a medicament for treating cancer, wherein one or more cancer cells express KRASG12C mutant protein.

143. The use of claim 141 or 142, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, melanoma, a solid tumor, or any combination of the foregoing.

144. An intermediate selected from:
(a) a compound of Formula (Int-AA): 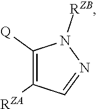
Formula (Int-AB): 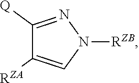
Formula (Int-AC): 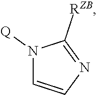
Formula (Int-AD): 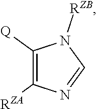
Formula (Int-AE): 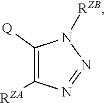
Formula (Int-AF): 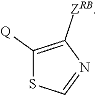
Formula (Int-AG): 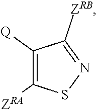
Formula (Int-AH): 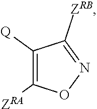
Formula (Int-AI): 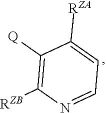
or Formula (Int-AJ): 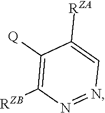
or a pharmaceutically acceptable salt of any of the foregoing; or
(b) a compound of Formula (Int-B): 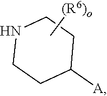
a nitrogen-protected analog thereof, or a pharmaceutically acceptable salt of any of the foregoing: or
(c) a compound of Formula (Int-C): 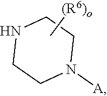
a nitrogen-protected analog thereof, or a pharmaceutically acceptable salt of any of the foregoing; or
(d) a compound of Formula (Int-D): 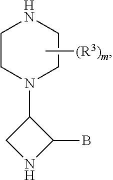
a nitrogen-protected analog thereof, or a pharmaceutically acceptable salt of any of the foregoing; or
(e) a compound of Formula (Int-E): 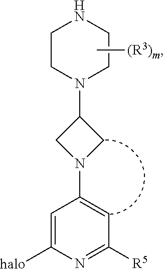
a nitrogen-protected analog thereof, or a pharmaceutically acceptable salt of any of the foregoing;
wherein:
A is 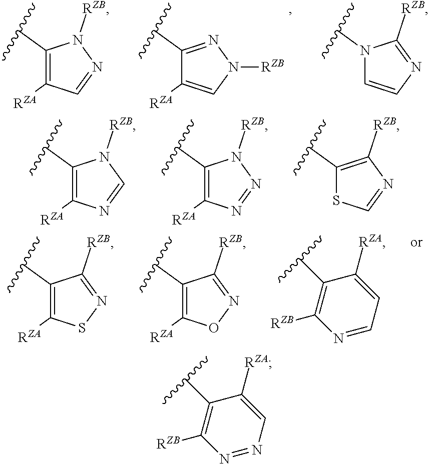
B is C₁₋₃alkylene-CH═CH₂ or C₁₋₃alkyleneOH;
Q is F, Cl, Br, I, or an organoborane;
m is 0, 1, 2, 3, or 4;
o is 0, 1, 2, 3, or 4;
halo is F, Cl, Br, or I; [symbol]
is C₂₋₆alkylene, C₃₋₆alkenylene, heteroalkylene having 2-6 total atoms and 1-3 heteroatoms selected from N, O, and S, or heteroalkenylene having 3-6 total atoms and 1 or 2 heteroatoms selected from N, O, and S, wherein [symbol] is unsubstituted or substituted with 1-4 substituents, and each substituent independently is C₁₋₃alkyl, C₁₋₃haloalkyl, C₂₋₃alkenyl, halo, CN, C₀₋₆alkyleneOH, C₀₋₆alkylene-C₁₋₃alkoxy, C₃₋₅cycloalkyl, C₄₋₅cycloalkenyl, heterocycloalkyl having 4 or 5 total ring atoms and 1-3 heteroatoms selected from N, O, and S, heterocycloalkenyl having 4 or 5 total ring atoms and 1-3 heteroatoms selected from N, O, and S, or phenyl; or two geminal substituents, together with the atom to which they are attached, form oxo, ═CH₂, spiro-C₃₋₅cycloalkyl, spiro-C₄₋₅cycloalkenyl, spiro-heterocycloalkyl having 3-5 total ring atoms and 1 or 2 heteroatoms selected from N, O and S, or spiro-heterocycloalkenyl having 4 or 5 total ring atoms and 1 or 2 heteroatoms selected from N, O and S; or two vicinal substituents, together with the atoms to which they are attached, form fused-C₃₋₅ cycloalkyl, fused-C₄₋₅cycloalkenyl, fused-heterocycloalkyl having 3-5 total ring atoms and 1 or 2 heteroatoms selected from N, O and S or fused-heterocycloalkenyl having 4 or 5 total ring atoms and 1 or 2 heteroatoms selected from N, O and S;
each of R^{ZA} and R^{ZB} independently is halo, CN, C₁₋₃alkyl, C₁₋₃haloalkyl, C₂₋₆alkenyl, C₂₋₆haloalkenyl, C₀₋₃alkylene-OH, C₀₋₆alkylene-C₁₋₃alkoxy, C₀₋₆alkylene-N(R^{N1})₂, C₀₋₂alkylene-C₃₋₆cycloalkyl, C₀₋₂alkylene-heterocycloalkyl having 3-6 total ring atoms and 1-3 heteroatoms selected from N, O, and S, or C₀₋₂alkylene-phenyl;
wherein each of the C₁₋₆alkyl, C₂₋₆alkenyl, C₀₋₆alkylene-C₁₋₃alkoxy, cycloalkyl, heterocycloalkyl, and phenyl substituents independently is unsubstituted or substituted with 1-3 further substituents, and each further substituent independently is D, halo, C₁₋₃alkyl, C₁₋₃haloalkyl, C₁₋₂alkyleneOH, C₁₋₂alkylene-C₁₋₃alkoxy, C₁₋₃deuterated alkoxy, N(R^{N1})₂, (C═O)C₁₋₃alkyl, C₃₋₅cycloalkyl, heterocycloalkyl having 3-5 total ring atoms and 1 or 2 heteroatoms selected from N, O, and S: or two geminal further substituents, together with the atom to which they are attached, form spiro-C₃₋₅cycloalkyl, or spiro-heterocycloalkyl having 3-5 total ring atoms and 1 or 2 heteroatoms selected from N, O, and S; or two vicinal further substituents, together with the atoms to which they are attached, form fused-C₃₋₅cycloalkyl or fused-heterocycloalkyl having 3-5 total ring atoms and 1 or 2 heteroatoms selected from N, O, and S; wherein each of the foregoing cycloalkyl and heterocycloalkyl further substituents independently is unsubstituted or substituted with 1 or 2 substituents, and each substituent independently is halo or C₁₋₃alkyl; and
each R^{N1} independently is H or C₁₋₃alkyl,
each R³ independently is C₁₋₃alkyl, C₁₋₃haloalkyl, 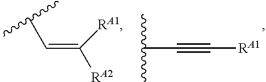
C₀₋₃alkyleneCN, C₀₋₃alkyleneOH, or C₀₋₃alkylene-C₁₋₃alkoxy; or two geminal R³, together with the atom to which they are attached, form oxo, spiro-C₃₋₇cycloalkyl, spiro-C₄₋₇cycloalkenyl, spiro-heterocycloalkyl having 3-7 total ring atoms and 1 or 2 heteroatoms selected from N, O, and S, or spiro-heterocycloalkenyl having 4-7 total ring atoms and 1 or 2 heteroatoms selected from N, O, and S; or two vicinal R³, together with the atoms to which they are attached, form fused-C₃-cycloalkyl, fused-C₄₋₇cycloalkenyl, fused-heterocycloalkyl having 3-7 total ring atoms and 1 or 2 heteroatoms selected from N, O, and S, or fused-heterocycloalkenyl having 4-7 total ring atoms and 1 or 2 heteroatoms selected from N, O, and S; and
R⁵ is halo, C₁₋₃haloalkyl, C₁₋₆alkyl, C₂₋₄alkenyl, C₂₋₃alkynyl, C₁₋₃alkoxy, C₁₋₃thioalkyl, C₃₋₇cycloalkyl, C₅₋₇cycloalkenyl, heterocycloalkyl having 3-7 total ring atoms and 1-3 heteroatoms selected from N, O, and S, or heterocycloalkenyl having 5-7 total ring atoms and 1-3 heteroatoms selected from N, O, and S, wherein each of the foregoing independently is unsubstituted or substituted with 1-3 substituents, and each substituent independently is C₁₋₃haloalkyl, C₀₋₆alkylene-OH, C₀₋₆alkylene-C₁₋₃alkoxy, C₃₋₇cycloalkyl, C₅₋₇cycloalkenyl, heterocycloalkyl having 3-7 total ring atoms and 1-3 heteroatoms selected from N, O, and S, heterocycloalkenyl having 4-7 total ring atoms and 1-3 heteroatoms selected from N, O, and S, or phenyl; and
each R⁶ independently is Br, Cl, F, CN, CH₃, CH₂F, CHF₂, CF₃, OH, CH₂OH, OCH₃, OCD₃, CH₂OCH₃, or CH₂N(CH₃)₂, or two geminal R⁶, together with the atom to which they are attached, form oxo, ═CH₂, spiro-cyclopropyl, spiro-cyclobutyl, spiro-oxetanyl, or spiro-tetrahydrofuranyl, or two vicinal R⁶, together with the atoms to which they are attached, form fused-cyclopropyl, fused-cyclobutyl, or fused-cyclopentyl, and any of the foregoing spiro and fused rings is unsubstituted or substituted with 1 or 2 substituents, and each substituent independently is halo, C₁₋₃alkyl, C₁₋₃haloalkyl, C₀₋₂alkyleneOH, C₀₋₂alkyleneC₁₋₃alkoxy, or C₀₋₂alkyleneCN.

145. The intermediate of claim 144, wherein:
B is CH₂CH═CH₂ or CH₂CH₂OH;
m is 0 or 1;
o is 0 or 1;
halo is Cl; [symbol]
is 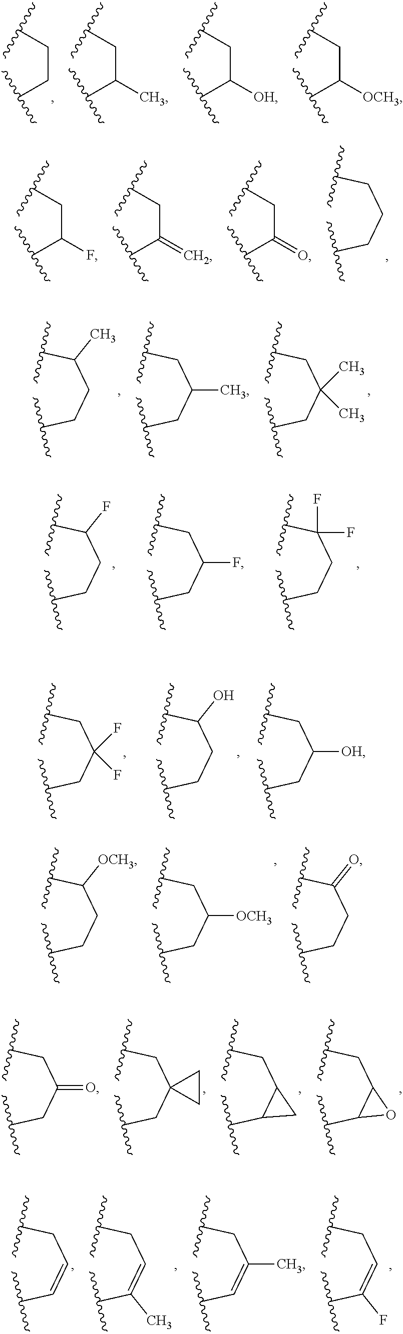 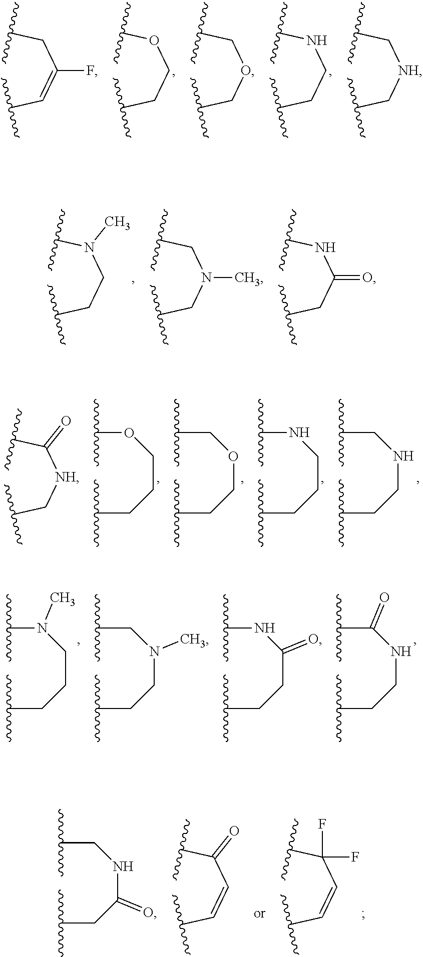
R³ is CH₃;
R⁵ is CHF₂ or CF₃:
Reis CH₃;
R^{ZA} is CH₃; and
R^{ZB} is CH₃, C(CH₃)₂CH₂OH, CH₂CH₂OCH₃, CH₂CH₂OCD₃, CH(CH₃)OCH₃,

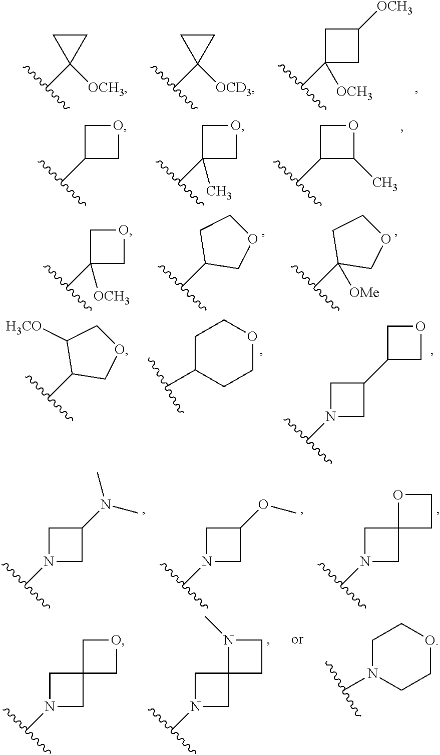

146. A compound listed in Table INT-A, Table INT-A′, Table INT-B, Table INT-C, Table INT-D, Table INT-E, Table INT-F, Table INT, a nitrogen protected analog thereof, or a pharmaceutically acceptable salt of any of the foregoing.

147. A process for preparing the compound or salt of any one of claims 43-129, comprising converting a compound or salt of any one of claims 144-146 into a compound or salt of any one of claims 43-129.
